(19) United States
(12) Patent Application Publication
(10) Pub. No.: US 2016/0040163 A1
(43) Pub. Date: Feb. 11, 2016

(54) DNAI FOR THE MODULATION OF GENES

(71) Applicant: PRONAI THERAPEUTICS, INC., Plymouth, MI (US)

(72) Inventors: Wendi Veloso RODRIGUEZA, Plymouth, MI (US); Mina Patel SOOCH, Plymouth, MI (US); Michael WOOLLISCROFT, Plymouth, MI (US); Rachel WEINGRAD, Plymouth, MI (US); Richard Adam MESSMANN, Plymouth, MI (US); Abhishek MANJUNATHAN, Plymouth, MI (US)

(21) Appl. No.: 14/777,214

(22) PCT Filed: Mar. 14, 2014
(86) PCT No.: PCT/US2014/029555

§ 371 Date: Sep. 15, 2015

Related U.S. Application Data
(60) Provisional application No. 61/794,778, filed on Mar. 15, 2013.

(51) Int. Cl. C12N 15/113; A61K 45/06; A61K 31/7088; A61K 9/127

(52) U.S. Cl. CPC C12N 15/113; A61K 9/127; A61K 45/06; A61K 31/7088; C12N 15/1135; C12N 2310/113; C12N 2310/531; C12N 2310/3341

(57) ABSTRACT

The present invention relates to methods and compositions for the inhibition of gene expression. In particular, the present invention provides oligonucleotide-based therapeutics for the inhibition genes implicated in many diseases.

### PRIORITY CLAIM

This application claims priority to U.S. Provisional Patent Application No. 61/794,778 filed on Mar. 15, 2013. The entire contents of the aforementioned application are incorporated herein by reference.

### FIELD OF THE INVENTION

The present invention relates to methods and compositions for the inhibition of gene expression. In particular, the present invention provides oligonucleotide-based therapeutics for the inhibition or interference of genes involved and implicated in diseases and cell systems.

### SEQUENCE LISTING

This application incorporates by reference in its entirety the Sequence Listing entitled “DNAi13728_ST25.txt” (2.90 MB), which was created Mar. 14, 2014 and filed electronically herewith.

### BACKGROUND OF THE INVENTION

The expression of gene products in cancer, e.g. oncogenes has become the central concept in understanding cancer biology and may provide valuable targets for therapeutic drugs. All oncogenes and their products operate inside the cell making protein-based drugs ineffective since their specificity involves ligand-receptor recognition.

Aside from oncogenes, proteins implicated in tumor suppression, genesis, progression, growth, proliferation, migration, cell cycle, cell signaling, metastases, invasion, transformation, differentiation, tolerance, vascular leakage, epithelial mesenchymal transition (EMT), aggregation, angiogenesis, adhesion, development of resistance, addiction to oncogenes and non-oncogenes (cytokines, chemokines, growth factors), alteration of immune surveillance or immune response, alteration of tumor stroma/local environment, endothelial activation, extracellular matrix remodeling, hypoxia and inflammation, immune activation or immune suppression, and survival and/or prevention of cell death by apoptosis, necrosis, or autophagy may be useful targets. Proteins implicated may be increased, decreased, or altered to have an impact on diseases and/or cell systems.

Similarly numerous protein products implicated (overexpressed, mutated, or suppressed) in non-cancerous diseases involving bacterial, cardiovascular (heart failure, atherosclerosis, dylipidemia, etc.), vascular, metabolic, diabetic, dental, oral, dermatological, endocrinology, fungal, gastroenterological, bowel (e.g. Crohn's, Ulcerative Colitis, or inflammatory bowel disease, etc.), genetic, hematological, hepatic, immunology, infections and/or infectious disease, inflammation (e.g. arthritis, etc.), musculosketal, nephrology, neurology (e.g. Alzheimer's, Parkinson's, Huntington's, Multiple Sclerosis, etc.), nutrition and/or weight loss, obstetrics/gynecology, ophthalmology, orthopedics, otolaryngology, pediatric/neonatology, podiatry, pulmonary/respiratory disease, rheumatology, sleep disorders, trauma, urology, stem cells, and viral (e.g. HCV, HIV, HBV, Herpes, etc.) may be useful targets.

Antisense oligonucleotides are under investigation as therapeutic compounds for specifically targeting oncogenes (Wickstrom, E. (ed). Prospects for antisense nucleic acid therapy of cancer and Aids. New York: Wiley-Liss, Inc. 1991; Murray, J. A. H. (ed). Antisense RNA and DNA New York: Wiley-Liss, Inc. 1992). Antisense drugs are modified synthetic oligonucleotides that work by interfering with ribosomal translation of the target mRNA. The antisense drugs developed thus far destroy the targeted mRNA by binding to it and triggering ribonuclease H (RNase H) degradation of mRNA. Oligonucleotides have a half-life of about 20 minutes and they are therefore rapidly degraded in most cells (Fisher, T. L. et al., Nucleic Acids Res. 21:3857-3865 (1993)). To increase the stability of oligonucleotides, they are often chemically modified, e.g., they are protected by a sulfur replacing one of the phosphate oxygens in the backbone (phosphorothioate) (Milligan, J. F. et al., J. Med. Chem. 36:1923-1937 (1993); Wagner, R. W. et al., Science 260:1510-1513 (1993)). However, this modification can only slow the degradation of antisense and therefore large dosages of antisense drug are required to be effective.

Despite the optimism surrounding the use of antisense therapies, there are a number of serious problems with the use of antisense drugs such as difficulty in getting a sufficient amount of antisense into the cell, non-sequence-specific effects, toxicity due to the large amount of sulfur containing phosphothioates oligonucleotides and their inability to enter their target cells, and their high cost due to continuous delivery of large doses. An additional problem with antisense drugs has been their nonspecific activities. Improvements to these first generation RNA targeted nucleic acid therapeutics utilize chemical modification to prevent degradation and utilize other modifications (e.g. 2′OMe modifications, CEt, locked nucleic acids (LNA), unlocked nucleic acids, inverted bases, conformationally-restricted nucleic acids (CRN)) to enable therapeutic windows of activity to be improved.

Other nucleic acid-based approaches beyond antisense also target RNA and its translational machinery rather than genomic DNA. These include double-stranded siRNA to block the translation of abberant proteins, RNA modulation to correct gene defects by exon skipping, and double or single-stranded microRNAs that function to regulate the expression of several gene pathways through the action of miRs and antimiRs, which replace absent sequences or antagonize sequences, respectively.

There is a need for additional non-protein based cancer therapeutics that target genes implicated in diseases. Therapeutics that are effective in low doses and that are non-toxic to the subject are particularly needed.

### SUMMARY OF THE INVENTION

The present invention relates to methods and compositions for the interference (inhibition, enhancement or alteration) of gene transcription or gene expression. In particular, the present invention provides oligonucleotide-based therapeutics for the modulation of disease causing genes.

An oligonucleotide that hybridizes to a non-coding region of a target gene, wherein the oligonucleotide comprises: a length of 20-34 bases; at least one CG pairs; at least 40% C and G content; no more than five consecutive bases of the same nucleotide; and may form at least one secondary structure. This oligonucleotide can also comprise a C and G content of at least 30% and in some embodiments the oligonucleotide comprises a C and G content of from about 50 to 80%. In some embodiments the oligonucleotide comprises at least two CG pairs. In some embodiments the oligonucleotide is complementary of said non-coding region of the target gene. In some embodiments the oligonucleotide is unique to the nucleotide sequence of the non-coding region. In some embodiments the nucleotide sequence of the non-coding region is not duplicated in a genome comprising the target gene. In some embodiments the nucleotide sequence of the non-coding region comprises 60% or greater homology to other nucleotide sequences in a genome with another gene. In some other embodiments the oligonucleotide is complementary to a non-coding region of another gene that influences that target gene. In yet other embodiments the oligonucleotide is complementary to a non-coding region of another gene that influences that target gene due to a chromosomal rearrangement. In yet other embodiments the oligonucleotide is complementary to a region upstream of the transcription start site.

In some embodiments, the present invention provides a composition comprising one or more distinct oligonucleotides that hybridizes under physiological conditions to regions upstream of the transcription start site of a disease causing gene.

In some embodiments, the region or regions upstream of the start site are located in regions on, surrounding or near transcription factor binding sites. In other embodiments, the regions are located on, surrounding or near various classes of regulatory elements (promoters, proximal promoters, distal enhancers, activators/co-activators, suppressors) that serve as cis-regulatory elements involved in gene transcription.

In some embodiments, the present invention provides compositions that are complementary to residues within CG regions. In some other embodiments, the present invention provides compositions that are complementary to residues within CpG islands. In yet other embodiments, the present invention resides in areas within nuclease hypersensitive areas.

In some embodiments, the present invention provides a composition comprising a first oligonucleotide that hybridizes under physiological conditions to the regulatory region of the target sequences. In some embodiments, at least one of the cytosine bases in the first oligonucleotide is 5-methylcytosine. In some of the embodiments, wherein at least one or all the cytosine bases in said CG pair is 5-methylcytosine. In some embodiments, all of the cytosine bases in the first oligonucleotide are 5-methylcytosine. In yet other embodiments, some of the bases in the first oligonucleotide are modified to prevent nuclease degradation during cell culture experiments. In some preferred embodiments, the hybridization of the first oligonucleotide to the promoter region of a gene modulates expression of the target gene. In some embodiments, the target gene is on a chromosome of a cell, and the hybridization of the first oligonucleotide to the regulatory region of the gene modulates cell signaling pathways of the cell. In some embodiments, the composition further comprises a second oligonucleotide. In some embodiments, at least one (e.g. all) of the cytosines in the second oligonucleotide are 5-methylcytosine.

In yet other embodiments, the present invention provides a method, comprising: providing an oligonucleotide; and a cell capable of transcription, and a cell capable of gene expression, and comprising a gene capable of being transcribed, and comprising a gene capable of being expressed; and introducing the oligonucleotide to the cell. In some embodiments, the introducing results in the modulation of the gene transcription. In some embodiments, the introducing results in the modulation of expression of the gene. In other embodiments, the introducing results in the modulation of proliferation of the cell. In yet other embodiments, the introducing results in the modulation of the cell phenotype. In certain embodiments, the introducing results in alteration of expression of other genes related to the target gene. In certain other embodiments, the introducing results in modulation of cell signaling pathways related to the target gene transcription. In yet other embodiments, the introducing results in an interference with the expression of other genes involved in transcription. In some embodiments, the cell is a cancer cell. In other embodiments, the cell is a prokaryote. In some other embodiments, the cell is a eukaryote. In some other embodiments the cell is in a host plant. In other embodiments, the cell is in a host animal (e.g., a non-human mammal or a human). In some embodiments, the oligonucleotide is introduced to the host animal at a dosage of between 0.1 mg to 10 g, and preferably at a dosage of between 00.1 mg to 100 mg per kg of body weight or 1 to 500 mg per meter squared body surface area. In some embodiments, the oligonucleotide is introduced to the host animal one or more times per day. In other embodiments, the oligonucleotide is introduced to the host animal continuously. In still further embodiments, the cell is in cell culture. In some embodiments, the method further comprises the step of introducing a test compound to the cell. In some embodiments, the test compound is a known chemotherapy or therapeutic agent. In some embodiments, the cancer is pancreatic cancer, colon/gastric cancer, breast cancer, renal/bladder cancer, lung cancer, leukemia, prostate, lymphoma, ovarian, thyroid cancer, sarcoma, or melanoma. In some embodiments, the non cancer disease involves bacterial, cardiovascular (heart failure, atherosclerosis, dylipidemia, etc.), vascular, metabolic, diabetic, dental, oral, dermatological, endocrinology, fungal, gastroenterological, bowel (e.g. Crohn's, Ulcerative Colitis, or inflammatory bowel disease, etc.), genetic, hematological, hepatic, immunology, infections and/or infectious disease, inflammation (e.g. arthritis, etc.), musculosketal, nephrology, neurology (e.g. Alzheimer's, Parkinson's, Huntington's, Multiple Sclerosis, etc.), nutrition and/or weight loss, obstetrics/gynecology, ophthalmology, orthopedics, otolaryngology, pediatric/neonatology, podiatry, pulmonary/respiratory disease, rheumatology, sleep disorders, trauma, urology, or viral (e.g. HCV, HIV, HBV, Herpes, etc.) disease.

In some embodiments, the method further provides a drug delivery system. In some embodiments, the drug delivery system comprises a nanoparticle, nanocrystal or complex, (e.g., a liposome comprising a neutral lipid or a lipid like compound or particles comprising polymer or polymer-like compound). In some embodiments, the drug delivery system comprises a cell targeting component (e.g., a ligand or ligand like molecule for a cell surface receptor or a nuclear receptor). In yet other embodiments, the drug delivery system comprises a device to administer the test compound(s). In certain embodiments, the drug delivery system is for use in vivo, and the oligonucleotide and the liposome, nanoparticle, nanocrystal or delivery system are present in the ratio of from 1:1 to 1:1000 (weight per weight).

The present invention further provides a composition comprising an oligonucleotide that hybridizes under physiological conditions to the coding strand of a gene under conditions such that expression of that gene is inhibited, enhanced or altered (i.e. modulated)

The present invention further provides a composition comprising an oligonucleotide that hybridizes under physiological conditions to the coding strand of a gene under conditions such that transcription of that gene is inhibited, enhanced or altered (i.e. modulated)

The present invention further provides a composition comprising an oligonucleotide that hybridizes under physiological conditions to the coding strand of a gene under conditions such that cell signaling pathways related to that gene is inhibited, enhanced or altered (i.e. modulated).

The present invention additionally provides a composition comprising an oligonucleotide that hybridizes under physiological conditions to the promoter region of a gene on a chromosome of a cell under conditions such that the cell phenotype is altered.

The present invention additionally provides a composition comprising an oligonucleotide that hybridizes under physiological conditions to the promoter region of a gene on a chromosome of a cell under conditions such that proliferation of the cell is reduced.

The present invention additionally provides a composition comprising an oligonucleotide that hybridizes under physiological conditions to the CG regions of a gene on a chromosome of a cell under conditions such that cell signaling pathways are modulated.

The present invention additionally provides a composition comprising an oligonucleotide that hybridizes under physiological conditions to CpG islands of a gene on a chromosome of a cell under conditions such that cell signaling pathways are modulated.

The present invention additionally provides a composition comprising an oligonucleotide that hybridizes under physiological conditions to the CG regions of a gene on a chromosome of a cell under conditions such that genes related to transcription of that gene are modulated.

The present invention additionally provides a composition comprising an oligonucleotide that hybridizes under physiological conditions to the CpG islands of a gene on a chromosome of a cell under conditions such that genes related to gene expression of that gene are modulated.

The present invention additionally provides a composition comprising an oligonucleotide that hybridizes under physiological conditions to the CG regions of a gene on a chromosome of a cell under conditions such that genes related to cell phenotype are modulated.

The present invention additionally provides a composition comprising an oligonucleotide that hybridizes under physiological conditions to the CpG islands of a gene on a chromosome of a cell under conditions such that genes related to cell phenotype are modulated.

The present invention additionally provides a method of inhibiting the expression of a gene in a subject (e.g., for the treatment of cancer or other hyperproliferative/overexpressive gene disorders) comprising providing an oligonucleotide that hybridizes under physiological conditions to the coding strand of a gene involved in cancer or a hyperproliferative/overexpressive gene disorder expressed in the biological sample, the oligonucleotide comprising at least on CG dinucleotide pair; and administering the oligonucleotide to the subject under conditions such that transcription or expression of the gene is inhibited, enhanced or altered (i.e. modulated). In some embodiments, the subject is a human.

In some embodiments, the method further provides a drug delivery system. In some embodiments, the drug delivery system comprises a liposome (e.g., a liposome comprising a neutral lipid or a lipid like compound or particles comprising polymer or polymer-like compound). In some embodiments, the drug delivery system comprises a cell targeting component (e.g., a ligand or ligand like molecule for a cell surface receptor or a nuclear receptor). In certain embodiments, the drug delivery system is for use in vivo, and the oligonucleotide and the liposome, nanoparticle, nanocrystal or delivery system are present in the ratio of from 1:1 to 1:1000 (weight per weight).

The present invention additionally provides a composition comprising an oligonucleotide that hybridizes under physiological conditions to the promoter region of a gene located on a chromosome of a cell under conditions such that transcription, phenotype or cell signaling pathways related to the target gene are modulated.

In certain embodiments, the present invention provides a kit comprising an oligonucleotide that hybridizes under physiological conditions to the promoter region of a gene, the oligonucleotide comprising at least one CG dinucleotide pair, wherein at least one of the cytosine bases in the CG dinucleotide pair comprises 5-methylcytosine; and instructions for using the kit for reducing proliferation of a cell comprising a gene on a chromosome of the cell or inhibiting gene expression. In some embodiments, the composition in the kit is used for treating cancer in a subject and the instructions comprise instructions for using the kit to treat cancer in the subject. In some embodiments, the instructions are instructions required by the U.S. Food and Drug Agency for labeling of pharmaceuticals.

The present invention also provides a method, comprising: providing a biological sample from a subject diagnosed with a cancer; and reagents for detecting the present or absence of expression of a oncogene in the sample; and detecting the presence or absence of expression of an oncogene in the sample; administering an oligonucleotide that hybridizes under physiological conditions to the promoter region of an oncogene expressed in the biological sample, the oligonucleotide comprising at least one CG dinucleotide pair.

The present invention additionally provides a method of inhibiting the expression of a gene in a subject (e.g., for the treatment of cancer or other hyperproliferative disorders) comprising providing an oligonucleotide that hybridizes under physiological conditions to the promoter region of a gene involved in cancer or a hyperproliferative disorder expressed in the biological sample, the oligonucleotide comprising at least one CG dinucleotide pair; and administering the oligonucleotide to the subject under conditions such that expression of the gene is inhibited. In some embodiments, the subject is a human.

The present invention additionally provides a method of modulating the transcription of a gene in a subject (e.g., for the treatment of disease) comprising an oligonucleotide that hybridizes under physiological conditions to the non-coding region of a gene involved in disease expressed in the biological sample, the oligonucleotide comprising at least one CG dinucleotide pair; and administering the oligonucleotide to the subject under conditions such that expression of the gene is inhibited. In some embodiments, the subject is a human.

In yet further embodiments, the present invention provides a method of screening compounds providing a cell comprising a suspected gene; and an oligonucleotide that hybridizes to the promoter region of the gene; and administering the oligonucleotide to the cell; and determining if the phenotype of the cell is modulated in the presence of the oligonucleotide relative to the absence of the oligonucleotide. In some embodiments, the cell is in culture (e.g., a prokaryote or eukaryote cell line). In other embodiments, the cell is in a host animal (e.g., a non-human mammal). In some embodiments, the method is a high-throughput screening method.

In other embodiments, the present invention relates to methods and compositions for cancer therapy. In particular, the present invention provides nanoparticle, nanocrystal, liposome, or complex based cancer or non-cancer therapeutics.

Accordingly, in some embodiments, the present invention provides a pharmaceutical composition comprising (e.g., consisting of) a cationic, neutral, or anionic lipids, polymers or delivery agents in a complex or mixture with an oligonucleotide. In some preferred embodiments, the liposome is cationic, neutral, anionic or amphoteric (e.g. SMARTICLES) in charge. In some preferred embodiments, the complex is a mixture of lipids, lipid-like, polymer or polymer-like delivery agents and a cation (e.g. lipids and calcium to form cochleates) or a mixture of lipids lipids, lipid-like, polymer or polymer-like delivery agents and an anion.

In some embodiments, the present invention provides a kit, comprising an oligonucleotide (e.g., an oligonuculeotide that hybridizes to the CG regions, CpG islands or promoter region of an onocogene) and a first pharmaceutical composition comprising (e.g., consisting of) a cationic, neutral, or anionic liposome comprises an optional second pharmaceutical composition, wherein the second pharmaceutical composition comprises a known chemotherapy agent (e.g., TAXOTERE, TAXOL, or VINCRISTINE, etc.), or chemotherapy cocktail, and wherein the known chemotherapy agent is formulated separately from the first pharmaceutical composition. In some embodiments, the chemotherapy agent is present at less than one half the standard dose, more preferably less than one third, even more preferably less than one fourth and still more preferable less than one tenth, and yet more preferably less than one hundredth the standard dose.

In some embodiments, the present invention provides a kit, comprising an oligonucleotide (e.g., an oligonuculeotide that hybridizes to the CG regions, CG islands, or promoter region of an onocogene) and a first pharmaceutical composition comprising (e.g., consisting of) a cationic, neutral, or anionic liposome comprises an optional second pharmaceutical composition, wherein the second pharmaceutical composition comprises a known agent (e.g., an antibiotic, an antiviral, an anti-inflammatory, etc.), or treatment cocktail, and wherein the known agent is formulated separately from the first pharmaceutical composition. In some embodiments, the agent is present at less than one half the standard dose, more preferably less than one third, even more preferably less than one fourth and still more preferable less than one tenth, and yet more preferably less than one hundredth the standard dose.

In yet other embodiments, the present invention provides a method, comprising providing a pharmaceutical composition consisting of a cationic, neutral, or anionic liposome and an oligonucleotide (e.g., an oligonuculeotide that hybridizes to the promoter region of an onocogene); and exposing the pharmaceutical composition to a cancer cell. In some preferred embodiments, the liposome is a cardiolipin based cationic liposome (e.g., NEOPHECTIN). In some preferred embodiments, the charge ration of NEOPHECTIN to oligonucleotide is 6:1. In other embodiments, the liposome comprises N-[1-(2,3-Dioleoyloxy)propyl]-N,N,N-trimethylammonium methyl-sulfate (DOTAP). In some embodiments, the cancer cell is a prostate cancer cell, an ovarian cancer cell, a breast cancer cell, a leukemia cell, or lymphoma cell. In some embodiments, the cell is in a host animal (e.g., a human). In some embodiments, the pharmaceutical composition is introduced to the host animal one or more times per day (e.g., continuously). In some embodiments, the method further comprises the step of administering a known chemotherapeutic agent to the subject (e.g., TAXOTERE, TAXOL, or VINCRISTINE), wherein the known chemotherapeutic agent is formulated separately from the cationic, neutral or anionic liposome. In preferred embodiments, the known chemotherapeutic agent is administered separately from the pharmaceutical composition. In some embodiments, the chemotherapy agent is present at less than one half the standard dose, more preferably less than one third, even more preferably less than one forth and still more preferable less than one tenth, and yet more preferably less than one hundredth the standard dose.

### DESCRIPTION OF THE FIGURES

FIG. 1 demonstrates a dose-dependent response for representative olionucleotides in MDA-MB-231 a human breast cell line.

FIG. 2 demonstrates a dose-dependent response for representative olionucleotides in A549 (human lung cell line).

FIG. 3 demonstrates a dose-dependent response for representative olionucleotides in DU145 (human prostate cell line).

FIG. 4 demonstrates a dose-dependent response for representative olionucleotides in MCF7 (human mammary breast cell line).

FIG. 5 depicts the structure of the olionucleotide SU1.

FIG. 6 depicts the structure of the olionucleotide SU2.

FIG. 7 depicts the structure of the olionucleotide SU3.

FIG. 8 depicts the structure of the olionucleotide SU1_{—}02.

FIG. 9 depicts the structure of the olionucleotide SU1_{—}03.

FIG. 10 demonstrates target inhibition of representative olionucleotides in DU145 (human prostate cell line).

FIG. 11 demonstrates target inhibition of representative olionucleotides in HCT-116 (human colorectal carcinoma).

FIG. 12 depicts the structure of the olionucleotide BE1.

FIG. 13 depicts the structure of the olionucleotide BE2.

FIG. 14 demonstrates target inhibition of representative olionucleotides in MDA-MB-231 a human breast cell line.

FIG. 15 demonstrates target inhibition of representative olionucleotides in DU145 (human prostate cell line).

FIG. 16 depicts the structure of the olionucleotide ST1.

FIG. 17 depicts the structure of the olionucleotide ST2.

FIG. 18 demonstrates target inhibition of representative olionucleotides in MDA-MB-231 a human breast cell line.

FIG. 19 demonstrates target inhibition of representative olionucleotides in DU145 (human prostate cell line).

FIG. 20 depicts the structure of the olionucleotide HI1.

FIG. 21 depicts the structure of the olionucleotide HI2.

FIG. 22 demonstrates target inhibition of representative olionucleotides in MDA-MB-231 a human breast cell line.

FIG. 23 demonstrates target inhibition of representative olionucleotides in DU145 (human prostate cell line).

FIG. 24 depicts the structure of the olionucleotide IL8-1.

FIG. 25 depicts the structure of the olionucleotide IL8-3.

FIG. 26 demonstrates target inhibition of representative olionucleotides in BxPC3 (human pancreatic cancer cell line).

FIG. 27 demonstrates target inhibition of representative olionucleotides in A549 (human lung cancer cell line).

FIG. 28 depicts the structure of the olionucleotide KR1.

FIG. 29 depicts the structure of the olionucleotide KR2.

FIG. 30 depicts the structure of the olionucleotide KR0525.

FIG. 31 demonstrates target inhibition of representative olionucleotides in MCF7 (human mammary breast cell line).

FIG. 32 depicts the structure of the olionucleotide IL6.

FIG. 33 demonstrates target inhibition of representative olionucleotides in HCT-116 (human colorectal carcinoma).

FIG. 34 depicts the structure of the olionucleotide AKT4

FIG. 35 demonstrates target inhibition of representative olionucleotides in MCF7 (human mammary breast cell line).

FIG. 36 depicts the structure of the olionucleotide BC1.

FIG. 37 demonstrates target inhibition of representative olionucleotides in HCT-116 (human colorectal carcinoma).

FIG. 38 depicts the structure of the olionucleotide MEK1_{—}1.

FIG. 39 depicts the structure of the olionucleotide MEK1_{—}2.

FIG. 40 demonstrates target inhibition of representative olionucleotides in HCT-116 (human colorectal carcinoma).

FIG. 41 depicts the structure of the olionucleotide MEK2_{—}1.

FIG. 42 demonstrates target inhibition of representative olionucleotides in MCF7 (human mammary breast cell line).

FIG. 43 depicts the structure of the olionucleotide WNT1_{—}1.

FIG. 44 depicts the structure of the olionucleotide WNT1_{—}2.

FIG. 45 depicts the structure of the olionucleotide WNT1_{—}3.

FIG. 46 demonstrates target inhibition of representative olionucleotides in MCF7 (human mammary breast cell line).

FIG. 47 depicts the structure of the olionucleotide EZH2_{—}2.

FIG. 48 demonstrates target inhibition of representative olionucleotides in MCF7 (human mammary breast cell line).

FIG. 49 depicts the structure of the olionucleotide PD1.

FIG. 50 demonstrates target inhibition of representative olionucleotides in MDA-MB-231 a human breast cell line.

FIG. 51 demonstrates target inhibition of representative olionucleotides in M14 (human melanoma cell line).

FIG. 52 demonstrates target inhibition of representative olionucleotides in NMuMG (a normal murine mouse mammary gland cell line).

FIG. 53 depicts the structure of the olionucleotide BL2.

FIG. 54 demonstrates target inhibition of representative olionucleotides in HCT-116 (human colorectal carcinoma).

FIG. 55 demonstrates target inhibition of representative olionucleotides in MCF7 (human mammary breast cell line).

FIG. 56 demonstrates target inhibition of representative olionucleotides in MDA-MB-231 a human breast cell line.

FIG. 57 demonstrates target inhibition of representative olionucleotides in MCF7 (human mammary breast cell line).

FIG. 58 depicts the structure of the olionucleotide CM7.

FIG. 59 depicts the structure of the olionucleotide CM12.

FIG. 60 depicts the structure of the olionucleotide CM13.

FIG. 61 depicts the structure of the olionucleotide CM14.

FIG. 62 demonstrates target inhibition of representative olionucleotides in MCF7 (human mammary breast cell line).

FIG. 63 depicts the structure of the olionucleotide TNF1.

FIG. 64 demonstrates target inhibition of representative olionucleotides in MCF7 (human mammary breast cell line).

FIG. 65 depicts the structure of the olionucleotide MIF1_{—}1.

FIG. 66 depicts the structure of the olionucleotide MIF1_{—}2.

FIG. 67 demonstrates that a representative oligonucleotide PC2 is capable of modulating target gene expression.

The figures are provided by way of example and are not intended to limit the scope of the present invention.

### DETAILED DESCRIPTION OF THE INVENTION

### Definitions

To facilitate an understanding of the present invention, a number of terms and phrases are defined below:

As used herein, the term “wherein said chemotherapy agent is present at less than one half the standard dose” refers to a dosage that is less than one half (e.g., less than 50%, preferably less than 40%, even more preferably less than 10% and still more preferably less than 1%) of the minimum value of the standard dosage range used for dosing humans. In some embodiments, the standard dosage range is the dosage range recommended by the manufacturer. In other embodiments, the standard dosage range is the range utilized by a medical doctor in the field. In still other embodiments, the standard dosage range is the range considered the normal standard of care in the field. The particular dosage within the dosage range is determined, for example by the age, weight, and health of the subject as well as the type of cancer being treated.

As used herein, the term “under conditions such that expression of said gene is modulated” refers to conditions where an oligonucleotide of the present invention hybridizes to a gene) and modulates expression of the gene by at least 10%, preferably at least 25% relative to the level of transcription in the absence of the oligonucleotide. The present invention is not limited to the modulation of expression of a particular gene. Exemplary genes include, but are not limited to Survivin, Beclin-1, STAT3, HIF1A, IL-8, KRAS, MTTP, ApoC III, ApoB, IL-17, MMP2, FAP, P-selectin, IL-6, IL-23, AKT, CRAF, Beta Catenin, PCSK9, MEK1, MEK2, CD4, WNT1, Clusterin, NRAS, EZH2, HDAC1, and PD-1, TNFα, MIF1, TTR, HBV, HAMP, ERBB2, PARP1, ITGA4, APP, FGFR1, CD68, ALK, MSI2, JAK2, CCND1. As used herein, the term “under conditions such that transcription of said gene is modulated” refers to conditions where an oligonucleotide of the present invention hybridizes to a gene and modulates transcription of the gene by at least 10%, preferably at least 25% relative to the level of transcription in the absence of the oligonucleotide. The modulation of transcription of said gene may involve related genes. The present invention is not limited to the modulation of expression of a particular gene.

As used herein the term “expression” is the process whereby information from a gene is used in the synthesis of a functional gene product. These products may be proteins, but in non-protein coding genes such as ribosomal RNA (rRNA), transfer RNA (tRNA) or small nuclear RNA (snRNA) genes, the product is a functional RNA or transcript to generate the macromolecular machinery for gene expression. Gene expression may be modulated at several levels including transcription, RNA splicing, translation, and post-translational modification of a protein. The term may also be used against a viral gene and refer to mRNA synthesis from a RNA molecule (i.e. RNA replication). For instance, the genome of a negative-sense single-stranded RNA virus may serve as a template to translate the viral proteins for viral replication afterwards.

As used herein the term “transcription” is the first step of gene expression where a segment of DNA is copied into RNA by RNA polymerase to produce a transcript. If the gene transcribed encodes a protein, the result of transcription is messenger RNA (mRNA) and expressed to produce a protein. Alternatively, a transcribed gene may encode for non-coding RNA genes (e.g. such as microRNA etc.), ribosomal RNA, transfer RNA (tRNA), other components of the protein-assembly process, or other ribozymes.

As used herein the term “phenotype” describes the modulation of gene expression to define the properties of the expression give rise to the organism's phenotype. A phenotype is expressed by proteins that control the organism's characteristics or traits, such as its morphology, shape, development, biochemical or physiological properties, and products that act to catalyze cell signaling and metabolic pathways characterizing the organism.

As used herein the term “cell signaling” describes a complex system of signals or pathways that governs cellular activities and coordinates cell actions. A cell's ability to perceive and respond to its environment is processed through proteins involved in the cell signaling pathway.

As used herein the term “CG regions” are regions of DNA where cytosine and guanine nucleotides are enriched in the linear sequence of bases along the length of a gene. Generally CG or GC percentage that is greater than 50% with an observed-to-expected CpG ratio that is greater than 60%. CG regions of DNA are also where a cytosine nucleotide occurs next to a guanine nucleotide and may be referred to as “CpG” for “C phosphodiester bond G”. Generally cytosine bases in CpGs are methylated.

As used herein the term “CpG islands” are regions of the genome that have high GC content and higher concentration of CpG sites associated with the start of the gene, promoter regions or regions 5′ upstream of a gene start site. CpG islands are typically 300-3,000 base pairs in length. CpG islands are recognized to be hypomethylated. In most instances the CpG sites in the CpG islands are unmethylated and may be recognized by HpaII restriction site, CCGG.

As used herein the term “nuclease hypersensitive site” is a short region of chromatin and is detected by its super sensitivity to cleavage by DNase I and other various nucleases. The nucleosomal structure is less compact, increasing the availability of the DNA to binding by proteins, such as transcription factors and DNase I. Hypersensitive sites are found on chromatin of cells associated with genes and generally precede active promoters. When DNA is transcribed, 5′ hypersensitive sites appear before transcription begins, and the DNA sequences within the hypersensitive sites are required for gene expression. Hypersensitive sites may be generated as a result of the binding of transcription factors.

As used herein “cis-regulatory element” is a region of DNA or RNA that regulates the expression of genes located on that same molecule of DNA A cis-regulatory element may be located upstream of the coding sequence of the gene it controls (in the promoter region or even further upstream), in an intron, or downstream of the gene's coding sequence, in either the translated or the untranscribed region. A cis-regulatory element may be located in another gene other than the target gene in instances of chromosomal rearrangements.

As used herein “non-coding” refers to a linear sequence of DNA that does not contribute to an amino acid sequence of a protein.

As used herein “Trinucleotide repeat expansion” refers to a triplet repeat expansion of DNA bases that causes any type of disorder categorized as a trinucleotide repeat disorder. Generally, the larger the expansion the more likely they are to cause disease or increase the severity of disease. Trinucleotide repeat disorders represent genetic by trinucleotide repeat expansion, a kind of mutation where trinucleotide repeats in certain genes exceed the normal, stable threshold, which differs per gene.

As used herein, the term “under conditions such that growth of said cell is reduced” refers to conditions where an oligonucleotide of the present invention, when administered to a cell (e.g., a cancer) reduces the rate of growth of the cell by at least 10%, preferably at least 25%, even more preferably at least 50%, and still more preferably at least 90% relative to the rate of growth of the cell in the absence of the oligonucleotide.

As used herein, the term “under conditions such that the expression of said target is modulated” refers to conditions where an oligonucleotide of the present invention, when administered to a cell (e.g., a cancer or non cancer or immune cell) modulates the expression of the protein by at least 10%, preferably at least 25%, relative to basal expression in the absence of the oligonucleotide.

The term “epitope” as used herein refers to that portion of an antigen that makes contact with a particular antibody.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “Delta G” or “ΔG” is the change in Gibbs Free Energy (in units of kcal/mole) and is the net exchange of energy between the system and its environment and can be described by the equation ΔG=ΔH−T·ΔS. Where ΔH (Enthalpy) represents the total energy exchange between the system and its surrounding environment (in units of kcal/mole), ΔS (Entropy) represents the energy spent by the system to organize itself (in units of cal/K·mole). Generally speaking a spontaneous system favors a more random system not an ordered system. Finally, T represents the absolute temperature of the system and is in units Kelvin (Celsius +273.15). The change of free energy is equal to the sum of its enthalpy plus the product of the temperature and entropy of the system. A positive ΔG reaction is generally non-spontaneous while a negative value is spontaneous.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc. and and non-vertebrate animals such as drosophila and nematode. In some embodiments, “non-human animals” further refers to prokaryotes and viruses such as bacterial pathogens, fungal, viral pathogens. Non-human animals is used broadly here to also indicate plants and plant genomes, especially commercially valuable crops such as corn, soybean, cotton, the grasses and legumes including rice and alfalfa as well as commercial flowers, vegetables and trees including deciduous and evergreen.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on the 5′ ends for a distance of about 1 kb or more such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, translocated, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. “Modulation” refers to regulation that is altered. Molecules (e.g., transcription factors) that are involved in up-regulation or down-regulation are often called “activators” and “repressors or suppressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) or phenotype when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 8 and 100), however, as used herein, the term is also intended to encompass longer polynucleotide chains (e.g., as large as 5000 residues). Oligonucleotides are often referred to by their length. For example a 24 residue or base oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

In some embodiments, oligonucleotides are “DNAi or DNA interference (DNAi).” As used herein, the term “DNAi” or refers to an oligonucleotide that hybridizes to region 5′ upstream of the transcription start site of a gene. In some embodiments, the hybridization of the DNAi or DNAi to the promoter modulates expression of the gene.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” or “100 percent” complementarity between the nucleic acids. The degree of complementarity between nucleic acid strands has significant effects on the efficiency and strength of hybridization between nucleic acid strands. The degree of complementarity is also defined the “native” sequence rather than having a mismatch. This is of particular importance in amplification reactions, as well as detection methods that depend upon binding between nucleic acids.

As used herein, the term “completely complementary,” for example when used in reference to an oligonucleotide of the present invention refers to an oligonucleotide where all of the nucleotides are complementary to a target sequence (e.g., a gene).

As used herein, the term “partially complementary,” for example when used in reference to an oligonucleotide of the present invention, refers to an oligonucleotide where at least one nucleotide is not complementary to the target sequence. Preferred partially complementary oligonucleotides are those that can still hybridize to the target sequence under physiological conditions. The term “partially complementary” refers to oligonucleotides that have regions of one or more non-complementary nucleotides both internal to the oligonucleotide or at either end. Oligonucleotides with mismatches at the ends may still hybridize to the target sequence.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C or C:G ratio within the nucleic acids. An oligonucleotide is a single molecule that contains a covalent bond linking each nucleotide and often pairing of complementary nucleic acids within its structure is said to be “self-hybridized” or having secondary structure.

As used herein the term “secondary structure” means a single molecule that contains a pairing of complementary nucleic acids within its structure that contributes to a two dimensional bend in said molecule.

As used herein, the term “linear section” refers to molecules with secondary structures wherein those secondary structures have regions of DNA that are not paired in a secondary manner they only have one covalent bond to the next oligonucleotide rather than both a bond and a pairing of complementary nucleic acids as one finds in regions having secondary structure.”

As used herein, the term “nuclease hypersensitive region” refers to regions of the target gene that are susceptible to oligonucleotide binding.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tm. The process of hybridization and dissociation is complex and highly dynamic and at the Tm, double strands are constantly formed and broken up, resulting in multiple interactions over time. The formation of secondary structures within an oligonucleotide may influence Tm.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4 H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH2PO4 H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH2PO4 H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The present invention is not limited to the hybridization of probes of about 500 nucleotides in length. The present invention contemplates the use of probes between approximately 8 nucleotides up to several thousand (e.g., at least 5000) nucleotides in length. One skilled in the relevant understands that stringency conditions may be altered for probes of other sizes (See e.g., Anderson and Young, Quantitative Filter Hybridization, in Nucleic Acid Hybridization [1985] and Sambrook et al., Molecular Cloning: A Laboratory Manual, Cold Spring Harbor Press, NY [1989]).

One skilled in the art would know numerous equivalent conditions may be employed to create low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “physiological conditions” refers to specific stringency conditions that approximate or are conditions inside an animal (e.g., a human). Exemplary physiological conditions for use in vitro include, but are not limited to, 37° C., 95% air, 5% CO2, commercial medium for culture of mammalian cells (e.g., DMEM media available from Gibco, Md.), 5-10% serum (e.g., calf serum or horse serum), additional buffers, and optionally hormone (e.g., insulin and epidermal growth factor).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

The term “mutant protein” as used herein to indicate that a protein containing a change in amino acid residues encoded by vector sequences that renders altered function or implicated in disease; that is, the mutant protein contains only those amino acids found in the protein as it occurs in nature. A mutant protein may be produced by recombinant means or may be isolated from a naturally occurring source

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, disorder of bodily function (e.g., cancer or non-cancer disease) or disrupt a system (e.g. cell culture). Test compounds comprise both known and potential therapeutic compounds. A test compound can be determined to be therapeutic by screening using the screening methods of the present invention. In some embodiments of the present invention, test compounds include antisense compounds.

As used herein, the term “known chemotherapeutic agents” refers to compounds known to be useful in the treatment of disease (e.g., cancer). Exemplary chemotherapeutic agents affective against cancer include, but are not limited to, daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin, lenolamide, and diethylstilbestrol (DES).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

“Hot Zones” in some embodiments, are regions within the promoter region of an oncogene are further defined as preferred regions for hybridization of oligonucleotides. In some embodiments, these preferred regions are referred to as “hot zones.” In some preferred embodiments, hot zones are defined based on oligonucleotide compounds that are demonstrated to be effective (see above section on oligonucleotides) and those that are contemplated to be effective based on the preferred criteria for oligonucleotides described above. Preferred hot zones encompass 20 bp upstream and downstream of each compound included in each hot zone and have at least 1 CG or more within an increment of 40 bp further upstream or downstream of each compound. In preferred embodiments, hot zones encompass a maximum of 100 bp upstream and downstream of each oligonucleotide compound included in the hot zone. In additional embodiments, hot zones are defined at beginning regions of each promoter. These hot zones are defined either based on effective sequence(s) or contemplated sequences and have a preferred maximum length of 1000 bp. Based on the above described criteria, exemplary hot zones were designed. Specific hot zones are described in the examples.

Combination and Single-Agent Therapy Using this DNAi Technology.

We present and define the following disease conditions as exemplary of, but not limited to, those that are potentially treatable with the DNAi therapeutic(s) described herein. Treatment of these disease entities may occur with single-agent DNAi therapy or DNAi therapy in combination with one or more therapeutics used to treat the conditions.

Cardiovascular Disease

Treating cardiovascular disease involves opening narrowed arteries, correcting abnormalities associated with irregular heartbeats and dysfunctional heart muscle or valves, reducing high blood pressure and high lipid levels, and amending imbalances in clotting that causes symptoms of pain and discomfort. Inventions may include: medical devices, dyslipidemics, antithrombotics, anticoagulants, anti-platelets, antihypertensives, anti-inflammatory, antihypertrophics, diuretics, anti-anginal, channel blockers, anti-restenosis agents, anti-atherosclerotics, anti-arrhythmics, enzyme inhibitors, and complement inhibitors.

Antianginals

The heart muscle works continuously and requires a constant supply of nutrients and oxygen. Those nutrients and oxygen are carried to the heart muscle in the blood. The chest pain known as angina can occur when there is an insufficient supply of blood, and consequently of oxygen, to the heart muscle. There are several types of antianginal medications. These include beta blockers (acebutolol, atenolol, betaxolol, bisoprolol, labetalol, metoprolol, nadolol, pindolol, propranolol, timolol), calcium channel blockers (diltiazem, nifedipine, verapamil), and vasodilators (nitroglycerin, isosorbide dinitrate). These drugs act by increasing the amount of oxygen that reaches the heart muscle.

Antiarrhythmics

Antiarrhythmics are used when the heart does not beat rhythmically or smoothly (a condition called arrhythmia), its rate of contraction must be regulated. Antiarrhythmic drugs (disopyramide, mexiletine, procainamide, propranolol, amiodarone, tocainide) prevent or alleviate arrhythmias by altering nerve impulses in the heart. Anticoagulants are used when clots develop on the interior wall of an artery block blood flow.

Antihyperlipidemics

Medications for treating atherosclerosis, or hardening of the arteries, act to reduce the serum levels of cholesterol and triglycerides, which form plaques on the walls of arteries. The following drug classes are used to treat high cholesterol or high lipid levels: HMG CoA reductase inhibitors (atorvastatin, simvastatin, lovastatin, and rosuvastatin, fluvastatin, pravastatin), fibrates (fenofibrate, gemfibrozil), bile acid sequestrants (cholestyramine, colestipol, and colesevelam), niacins (niacin, Vit B3, nicotinic acid), and cholesterol absorption inhibitors (ezetimide), or drug combinations of these classes.

Antihypertensives

High blood pressure is caused when the pressure of the blood against the walls of the blood vessels is higher than what is considered normal. High blood pressure, or hypertension, eventually causes damage to the brain, eyes, heart, or kidneys. Several different drug actions produce an antihypertensive effect. Some drugs block nerve impulses that cause arteries to constrict; others slow the heart rate and decrease its force of contraction; still others reduce the amount of a certain hormone in the blood that causes blood pressure to rise. The effect of any of these medications is to reduce blood pressure. The mainstay of antihypertensive therapy is often a diuretic, a drug that reduces body fluids. Examples of antihypertensive drugs include beta blockers, calcium channel blockers, ACE (angiotensin-converting enzyme) inhibitors (including benazepril, captopril, enalapril, lisinopril, and quinapril), and the agents valsartan, losartan, prazosin, and terazosin.

Antiplatelets

Antilatelet drugs alter the platelet activation at the site of vascular damage crucial to the development of arterial thrombosis. Aspirin irreversibly inhibits the enzyme COX, resulting in reduced platelet production of TXA2 (thromboxane—powerful vasoconstrictor that lowers cyclic AMP and initiates the platelet release reaction). Dipyridamole inhibits platelet phosphodiesterase, causing an increase in cyclic AMP with potentiation of the action of PGI2-—opposes actions of TXA2. Clopidogrel (Plavix) affects the ADP-dependent activation of IIb/IIIa complex. Glycoprotein IIb/IIIa receptor antagonists block a receptor on the platelet for fibrinogen and von Willebrand factor and include for example, abciximab eptifibatide and tirofiban. Epoprostenol is a prostacyclin that is used to inhibit platelet aggregation during renal dialysis (with or without heparin) and is also used in primary pulmonary hypertension.

Antithrombotics

An antithrombotic agent is a drug that reduces thrombus formation. These include plasminogen activators: Alteplase, Reteplase, Tenecteplase, Saruplase, Urokinase, Anistreplase, Monteplase, Streptokinase, other serine endopeptidases (Ancrod, Brinase, Fibrinolysin)

Beta Blockers

Beta-blocking medications block the response of the heart and blood vessels to nerve stimulation, thereby slowing the heart rate and lowering blood pressure. They are used in the treatment of a wide range of diseases, including angina, high blood pressure, migraine headaches, arrhythmias, and glaucoma. Metoprolol and propranolol are common beta blockers.

Calcium Channel Blockers

Calcium channel blockers (diltiazem, nifedipine, verapamil) are used for the prevention of angina (chest pain). Verapamil is also useful in correcting certain arrhythmias (heartbeat irregularities) and lowering blood pressure. This group of drugs is thought to prevent angina and arrhythmias and lower blood pressure by blocking or slowing calcium flow into muscle cells, which results in vasodilation (widening of the blood vessels) and greater oxygen delivery to the heart muscle.

Cardiac Glycosides

Cardiac glycosides include drugs that are derived from digitalis (digoxin is an example). This type of drug slows the rate of the heart but increases its force of contraction. Cardiac glycosides act as both heart depressants and stimulants: They may be used to regulate irregular heart rhythm or to increase the volume of blood pumped by the heart in heart failure.

Diuretics

Diuretic drugs, such as chlorothiazide, chlorthalidone, furosemide, hydrochlorothiazide, and spironolactone, promote the loss of water and salt from the body to lower blood pressure or increase the diameter of blood vessels. Antihypertensive medications cause the body to retain salt and water and are often used concurrently with diuretics. Most diuretics act directly on the kidneys, but there are different types of diuretics, each with different actions. This allows therapy for high blood pressure to be adjusted to meet the needs of individual patients.

Thiazide diuretics, such as chlorothiazide, chlorthalidone, and hydrochlorothiazide, are the most commonly prescribed and generally well tolerated as once or twice a day pills. A major drawback of thiazide diuretics is that they often deplete the body of potassium and therefore compensated with potassium supplements. Loop diuretics, such as furosemide, act more vigorously than thiazide diuretics. (Loop refers to the structures in the kidneys on which these specific diuretic medications act.) Loop diuretics promote more water loss than thiazide diuretics but they also deplete more potassium from the body. Potassium sparing diuretics are also used treat heart failure and high blood pressure and include amiloride, spironolactone, and triamterene. Generally drug combinations of amiloride and hydrochlorothiazide, spironolactone and hydrochlorothiazide, and triamterene and hydrochlorothiazide are used to enhance the antihypertensive effect and reduce potassium loss.

Vasodilators

Vasodilating medications cause the blood vessels to dilate, or widen. Some of the antihypertensive medications, such as hydralazine and prazosin, lower blood pressure by dilating the arteries or veins. Other vasodilating medicines are used in the treatment of stroke and diseases that are characterized by poor blood circulation. Ergoloid mesylates, for example, are used to reduce the symptoms of senility by increasing the flow of oxygen-rich blood to the brain.

Metabolic Disease (Diabetes)

Diabetes is usually a lifelong or chronic disease caused by high levels of sugar in the blood. Insulin is a produced by the pancreas to control blood sugar and diabetes can be caused by too little insulin, resistance to insulin, or both. There are several types of diabetes. (1) Type 1 diabetes can occur at any age, but it is most often diagnosed in children, teens, or young adults. It is caused by the destruction of islet cells in the pancreas resulting in little or no insulin thereby requiring daily injections of insulin. (2) Type 2 diabetes results from insulin resistance and relative insulin deficiency. Obesity is thought to be the primary cause of Type 2 diabetes in those genetically predisposed. (3) Gestational diabetes is high blood sugar that develops at any time during pregnancy in a woman who does not have diabetes.

The following treatments for diabetes include: insulin, biguanides (metformin), suphonylureas, nonsulfonylurea secretagogues, meglitinides/prandial glucose regulatory/glinides, alpha-glucosidase inhibitors, thiazolidineione/glitazones, glucagon-like peptide-1 analog, amylin analogues, and dipeptidyl peptidase-4 inhibitors.

Metformin is generally recommended as a first line treatment. When metformin is not sufficient another class is added.

Sulfonylureas lower blood sugar by stimulating the pancreas to release more insulin. The first drugs of this type that were developed—Dymelor (acetohexamide), Diabinese (chlorpropamide), Orinase (tolbutamide), and Tolinase (tolazamide)—are not as widely used since they tend to be less potent and shorter-acting drugs than the newer sulfonylureas. They include Glucotrol (glipizide), Glucotrol XL (extended release), DiaBeta (glyburide), Micronase (glyburide), Glynase PresTab (glyburide), and Amaryl (glimepiride). These drugs can cause a decrease in the hemoglobin A1c (HbA1c) of up to 1%-2%. Biguanides improve insulin's ability to move sugar into cells especially into the muscle cells and prevent the liver from releasing stored sugar. Biguanides are counterindicated in people who have kidney damage or heart failure because of the risk of precipitating a severe build-up of lactic acid (called lactic acidosis) in these patients. Biguanides can decrease the HbA1c 1%-2%. An example includes metformin (Glucophage, Glucophage XR, Riomet, Fortamet, and Glumetza).

Thiazolidinediones improve insulin's effectiveness (improving insulin resistance) in muscle and in fat tissue. They lower the amount of sugar released by the liver and make fat cells more sensitive to the effects of insulin. Actos (pioglitazone) and Avandia (rosiglitazone) are the two drugs of this class. A decrease in the HbA1c of 1%-2% can be seen with this class of oral diabetes medications. Thiazolidinediones should used with caution in people with heart failure. Avandia is restricted for use in new patients only if they are uncontrolled on other medications and are unable to take Actos.

Alpha-glucosidase inhibitors include Precose (acarbose) and Glyset (miglitol). These drugs block enzymes that help digest starches, slowing the rise in blood sugar. These diabetes pills may cause diarrhea or gas. They can lower hemoglobin A1c by 0.5%-1%.

Meglitinides include Prandin (repaglinide) and Starlix (nateglinide). These diabetes medicines lower blood sugar by stimulating the pancreas to release more insulin. The effects of these drugs are glucose-dependent, with high blood sugar inducing insulin release, which is unlike the action of sulfonylureas which cause insulin release, regardless of glucose levels, and can lead to hypoglycemia.

Dipeptidyl peptidase IV (DPP-IV) inhibitors include Januvia (sitagliptin), Nesina (alogliptin), Onglyza (saxagliptin), Galvus (vildagliptin) and Tradjenta (linagliptin). The DPP-IV inhibitors work to lower blood sugar in patients with type 2 diabetes by increasing insulin secretion from the pancreas and reducing sugar production. These diabetes pills increase insulin secretion when blood sugars are high. They also signal the liver to stop producing excess amounts of sugar. DPP-IV inhibitors control sugar without causing weight gain. The medication may be taken alone or with other medications such as metformin.

Glucagon-Like Peptide Analogs and Agonists

Glucagon-like peptide (GLP) agonists bind to a membrane GLP receptor. As a consequence, insulin release from the pancreatic beta cells is increased. Examples of this class include Exenatide (also Exendin-4, marketed as Byetta). Exenatide is not an analogue of GLP but rather a GLP agonist. Typical reductions in A1C values are 0.5-1.0%. Liraglutide, a once-daily human analogue (97% homology), has been developed by Novo Nordisk under the brand name Victoza. Taspoglutide is presently in Phase III clinical trials with Hoffman-La Roche.

Alpha-glucosidase inhibitors (Acarbose, Miglitol, Voglibose), amylin analogues (Pramlintide), SGLT2 inhibitors (Canagliflozin, Dapagliflozin, Empaliflozin, Remogliflozin, Sergliflozin) and others (Benfluorex, Tolrestat)

Combination agents are the combination of two medications in one tablet and include the following examples: Glucovance, which combines glyburide (a sulfonylurea) and metformin, Metaglip, which combines glipizide (a sulfonylurea) and metformin, and Avandamet which utilizes both metformin and rosiglitazone (Avandia). Kazano (alogliptin and metformin) and Oseni (alogliptin plus pioglitazone) are other examples.

Eye Disorders

Ocular Bacterial Infection. Antibiotics are generally used to treat, or sometimes to prevent a bacterial eye infection. Examples of common antibiotics used in the eye are sulfacetamide, erythromycin, gentamicin, tobramycin, ciprofloxacin and ofloxacin.

Ocular Inflammatory reaction. Anti-inflammatories reduce inflammation, which in the eye is usually manifest by pain, redness, light sensitivity and sometimes blurred vision. Anti-inflammatories can be either glucocorticoids/corticosteroids or NSAIDs. Corticosteroids are very effective anti-inflammatories for a wide variety of eye problems including all disorders associated with systemic inflammatory reactions (Reiter's syndrome, xerostomia, etc.). Common corticosteroids include: Prednisolone, Fluorometholone and Dexamethasone. Non-steroidal anti-inflammatories reduce the production of pro-inflammatory factors such as prostaglandins. Common NSAIDs include: Diclofenac, Ketorolac and Flurbiprofen.

Glaucoma. Glaucoma is a disorder of regulation of intraocular pressure. Glaucoma medications all attempt to reduce this pressure to prevent damage to the optic nerve resulting in loss of vision. These medications may lower pressure by decreasing the amount of fluid produced in the eye, by increasing the amount of fluid exiting through the eye's natural drain, or by providing additional pathways for fluid to leave the eye. More than one glaucoma medication is used simultaneously, as these effects can combine to lower pressure further than possible with a single medication. These medications are listed by class:

### BETA-BLOCKERS: Timolol, Metipranolol, Carteolol, Betaxolol, Levobunolol

### ALPHA AGONISTS: Brimonidine, Iopidine

### PROSTAGLANDIN ANALOGUES: Latanoprost

### CARBONIC ANHYDRASE INHIBITORS: Dorzolamide

### CHOLINERGIC AGONISTS: Pilocarpine, Carbachol

### ADENERGIC AGONISTS, Epinephrine, Dipivefrin

Ocular Viral Infection

Used primarily in treating herpes virus infections of the eye, antiviral eye medications may be used in conjunction with oral medications for elimination the virus. The most common type of antiviral is triflurthymidine. Other topical anti-virals include adenine arabinoside and idoxuridine.

Allergic Reaction

All anti-allergy topicals decrease the effects of histamine, a factor that mediates, the inflammatory reaction. Common anti-allergy medicines include livostin, patanol, Cromolyn and alomide.

Infectious Diseases

Aminoglycosides. This class of antibiotics is used to treat infections caused by Gram-negative bacteria, such as Escherichia coli and Klebsiella, particularly Pseudomonas aeruginosa. This class is also effective against Aerobic bacteria (but not obligate/facultative anaerobes) and in the treatment of tularemia. The mechanism of action includes binding to the bacterial 30S ribosome/ribosomal subunit (some work by binding to the 50S subunit), inhibiting the translocation of the peptidyl-tRNA from the A-site to the P-site and also causing misreading of mRNA, leaving the bacterium unable to synthesize proteins vital to its growth. Possible toxicities include hearing loss, vertigo and nephrotoxicity. Examples of aminoglycosides include Amikacin, Gentamicin, Kanamycin, Neomycin, Netilmicin, Tobramycin, Paromomycin, Spectinomycin.

Ansamycins. Used as anti-tumor antibiotics and for treatment of traveler's diarrhea caused by E. coli. Examples include Geldanamycin, Herbimycin, and Rifaximin.

Carbacephem. This class prevents bacterial cell division by inhibiting cell wall synthesis. An example is Loracarbef.

Carbapenem. This class works by inhibiting cell wall synthesis. It is bactericidal for both Gram-positive and Gram-negative organisms and therefore useful for empiric broad-spectrum antibacterial coverage. (Note MRSA resistance to this class.). Toxicity may include gastrointestinal upset and diarrhea, nausea, seizures, headache, rash and allergic reactions. Examples include Ertapenem, Doripenem, Imipenem/Cilastatin, Meropenem.

Cephalosporins (First generation). Have the same mode of action as other beta-lactam antibiotic to disrupt the synthesis of the peptidoglycan layer of bacterial cell walls. The class provides good coverage against Gram positive infections. Potential toxicities include gastrointestinal upset and diarrhea, nausea (if alcohol taken concurrently) and allergic reactions. Examples include Cefadroxil, Cefazolin, Cefalotin, Cefalothin, Keflin, and Cefalexin.

Cephalosporins (Second generation). This class provides less gram-positive coverage than the above with improved gram negative cover. They have the same mode of action as other beta-lactam antibiotics and disrupt the synthesis of the peptidoglycan layer of bacterial cell walls. They may cause gastrointestinal upset and diarrhea, nausea (if alcohol taken concurrently) and allergic reactions. Examples include: Cefaclor, Cefamandole, Cefoxitin, Cefprozil and Cefuroxime.

Cephalosporins (Third generation). Same mode of action as other beta-lactam antibiotic to disrupt the synthesis of the peptidoglycan layer of bacterial cell wall. Provides improved coverage of Gram-negative organisms, except Pseudomonas. Has reduced Gram-positive coverage. May cause gastrointestinal upset and diarrhea, nausea (if alcohol taken concurrently and allergic reactions. Examples include Cefixime, Cefdinir, Cefditoren, Cefoperazone, Cefotaxime, Cefpodoxime, Ceftazidime, Ceftibuten, Ceftizoxime, and Ceftriaxone.

Cephalosporins (Fourth generation). As above for mechanism and toxicity but good coverage for pseudomonal infections. Examples include Cefepime.

Cephalosporins (Fifth generation). As above for mechanism and toxicity but good coverage for Methicillin-resistant Staphylococcus aureus/MRSA. Examples include Ceftaroline fosamil, and Ceftobiprole.

Glycopeptides Inhibit peptidoglycan synthesis and are active against aerobic and anaerobic Gram positive bacteria including MRSA; Vancomycin is used orally for the treatment of C. difficile. Examples include Teicoplanin, Vancomycin, and Telavancin

Lincosamides. Bind to 50S subunit of bacterial ribosomal RNA thereby inhibiting protein synthesis. Used to treat serious staph-, pneumo-, and streptococcal infections in penicillin-allergic patients, also anaerobic infections; clindamycin topically used for acne and possible C. difficile-related pseudomembranous enterocolitis. include Clindamycin and Lincomycin.

Lipopeptides. Bind to the membrane and cause rapid depolarization, resulting in a loss of membrane potential leading to inhibition of protein, DNA and RNA synthesis Gram-positive organisms. Example is Daptomycin.

Macrolides. Are enzyme inhibitors of bacterial protein biosynthesis by binding reversibly to the subunit 50S of the bacterial ribosome, thereby inhibiting translocation of peptidyl-tRNA. Used to treat Streptococcal infections, syphilis, upper respiratory tract infections, lower respiratory tract infection, mycoplasmal infections, Lyme disease. Can cause nausea, vomiting, and diarrhea (especially at higher doses), prolonged QT interval (especially erythromycin) and Jaundice. Examples include Azithromycin, Clarithromycin, irithromycin, Erythromycin, Roxithromycin, Troleandomycin, Telithromycin and Spiramycin.

Monobactams. Same mode of action as other beta-lactam antibiotics, to disrupt the synthesis of the peptidoglycan layer of bacterial cell walls. Example includes Aztreonam.

Nitrofurans. Are used to treat bacterial or protozoal diarrhea or enteritis. An example is Furazolidone and Nitrofurantoin to treat urinary tract infections.

Oxazolidonones. Protein synthesis inhibitors, they prevent the initiation step and are used to treat vancomycin-resistant Staphylococcus aureus. Can cause thrombocytopenia, and peripheral neuropathy. Examples include Linezolid, Radezolid,

Penicillins. Disrupt the synthesis of the peptidoglycan layer of bacterial cell walls.

These are used to treat a wide range of infections; penicillin is used for streptococcal infections, syphilis and Lyme disease and can cause gastrointestinal upset and diarrhea, allergy with serious anaphylactic reaction, brain and kidney damage (rare). Examples include, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin.

Penicillin combinations. The second component prevents bacterial antibiotic resistance to the first component. Examples include Augmentin, Ampicillin/sulbactam, Piperacillin/tazobactam, Ticarcillin/clavulanate.

Polypeptide antibiotics. For treatment of eye, ear or bladder infections; usually applied directly to the eye or inhaled into the lungs; rarely given by injection, although the use of intravenous colistin is experiencing a resurgence due to the emergence of multi drug resistant organisms. This class can cause kidney and nerve damage (when given by injection). The class inhibits isoprenyl pyrophosphate, a molecule that carries the building blocks of the peptidoglycan bacterial cell wall outside of the inner membrane. Examples include Bacitracin, Colistin, and Polymyxin B

Quinolones. For treatment of urinary tract infections, bacterial prostatitis, community-acquired pneumonia, bacterial diarrhea, mycoplasmal infection, gonorrhea. Can cause nausea (rare), irreversible damage to central nervous system (uncommon), tendinosis (rare). The class works by inhibiting the bacterial DNA gyrase or the topoisomerase IV enzyme, thereby inhibiting DNA replication and transcription. Examples include, Ciprofloxacin, Enoxacin, Gatifloxacin, Levofloxacin, Lomefloxacin, Moxifloxacin, Avelox, Nalidixic acid, Norfloxacin, Ofloxacin, Trovafloxacin, Grepafloxacin, Raxar, Sparfloxacin and Temafloxacin.

Sulfonamides. They are competitive inhibitors of the enzyme dihydropteroate synthetase, DHPS. DHPS catalyses the conversion of PABA (para-Aminobenzoic acid) to dihydropteroic acid|dihydropteroate, a key step in folate synthesis. Folate is necessary for the cell to synthesize nucleic acids (nucleic acids are essential building blocks of DNA and RNA, and in its absence cells will be unable to divide. The class is used to treat Urinary tract infections (except sulfacetamide, used for Conjunctivitis, and mafenide and silver sulfadiazine, used topically for burns. The class can cause nausea, vomiting, and diarrhea, Allergy, including skin rashes, crystals in urine, Renal failure, decrease in white blood cell count and sensitivity to sunlight. Examples include Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, and Trimethoprim-Sulfamethoxazole.

Tetracyclines Inhibit the binding of aminoacyl-tRNA to the mRNA-ribosome complex. They do so mainly by binding to the 30S ribosomal subunit in the mRNA translation complex. Can be used to treat Syphilis, Chlamydia infections, Lyme disease, mycoplasmal infections, acne, rickettsial infections, and malaria caused by a protest and not a bacterium. Toxicity includes Gastrointestinal upset, Sensitivity to sunlight, Potential toxicity to mother and fetus during pregnancy, Enamel hypoplasia (staining of teeth; potentially permanent, transient depression of bone growth. Examples include Demeclocycline, Doxycycline, Minocycline, Oxytetracycline, and Tetracycline.

Drugs against mycobacteria include the following: Clofazimine, Dapsone, Capreomycin, Cycloserine, Ethambutol, Ethionamide, Isoniazid, Pyrazinamide, Rifampicin, Rifabutin, Rifapentine, Streptomycin, and aminoglycosides.

Other antibiotics include the following:

Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim. Anti-Viral Medications by Indication

Herpes Simplex Virus (HSV), Varicella Zoster Virus (VZV) and cytomegalovirus (CMV). Oral herpes simplex virus (HSV) causes mucous membrane lesions (i.e., cold sores), and genital HSV causes genital herpetic lesions. Treatment for HSV can also be used for the treatment of Varicella Zoster Virus (VZV) the causative agent for chicken-pox in children and shingles in adults. Typical anti-virals include Acyclovir and Valaciclovir, both inhibitors of viral DNA synthesis. Additionally, Idoxuridine and Brivudin can be incorporated into the viral DNA leading to a hindered mechanism of DNA duplication. A third type of herpes viruses with established treatment is cytomegalovirus (CMV), particularly dangerous for unborn children, infants and immune-compromised patients. Medications used to treat CMV are Ganciclovir and Foscarnet, also indicated in some HSV infections. They act to inhibit viral DNA synthesis.

HIV. A diverse group of antiviral medications control viral load, but cannot cure HIV infections. Viral entry inhibitors such as Enfuvirtide prevent newly formed viruses from entering uninfected host cells by preventing virus-cell fusion.

Reverse transcriptase inhibitors include many drugs such as Abacavir, Lamivudine, Zidovudine, Tenofovir, Efavirenz and Nevirapine. These drugs inhibit reverse transcriptase, an enzyme critical to the mechanism by which HIV transcribes genetic material.

Another anti-viral approach utilizes the protease inhibitors such as Atazanavir, Indinavirn and Ritonavir to inhibit assembly of new viruses. Combination therapies using 2 or 3 of the aforementioned agents are very effective at reducing serum viral load to below detectable levels.

Hepatitis. One of the few anti-HBV (hepatitis B) medications is Lamivudine, a reverse transcriptase inhibitor. Additionally, adefovir and dipivoxil, medications used in the treatment of HIV can be used to inhibit transcription of viral HBV RNA into DNA. Interferons are naturally occurring molecules that stimulate immune responses against invading species, including viral particles. Imiquimod up-regulates the natural production of interferons to boost the human immune response. Synthetically produced Alpha-interferon is also effective in treating HBV and HCV, especially in combination with other drugs. Unfortunately, interferons are associated with a number of severe toxicities that limit their long-terms usage in a number of patients.

Broad-spectrum Antiviral Medications

Ribavirin is effective in the treatment of influenza, HCV and paramyxoviruses such as measles and respiratory syncytial virus by blocking synthesis of viral RNA. A combination of Ribavirin and Alfa-interferon is proven to be effective in treatment of chronic hepatitis C infections.

Inflammation. Anti-Inflammatory medications by class

Glucocorticoids. This class of anti-inflammatory medication reduces inflammation by binding to glucocorticoid receptors (GR). The activated GR complex, in turn, up-regulates the expression of anti-inflammatory proteins in the nucleus (a process known as transactivation) and represses the expression of pro-inflammatory proteins in the cytosol by preventing the translocation of other transcription factors from the cytosol into the nucleus. These drugs are often referred to as corticosteroids. Examples include Budesonide, cortisone, dexamethasone, hydrocortisone, methylprednisolone, prednisolone and prednisolone.

Non-steroidal anti-inflammatory drugs (NSAIDs). NSAIDs reduce inflammation by reducing the production of prostaglandins, chemicals that promote inflammation, pain, and fever. Prostaglandins also protect the lining of the stomach and intestines from the damaging effects of acid, and promote blood clotting by activating blood platelets and affect kidney function. The enzymes that produce prostaglandins are called cyclooxygenase (COX). There are two types of COX enzymes, COX-1 and COX-2. Both enzymes produce prostaglandins that promote inflammation, pain, and fever; however, only COX-1 produces prostaglandins that activate platelets and protect the stomach and intestinal lining. NSAIDs block COX enzymes and reduce production of prostaglandins. Therefore, inflammation, pain, and fever are reduced. Since the prostaglandins that protect the stomach and promote blood clotting also are reduced, NSAIDs can cause ulcers in the stomach and intestines, and increase the risk of bleeding. Aspirin is the only NSAID that inhibits the clotting of blood for a prolonged period of time, four to seven days, and is therefore effective for preventing blood clots that cause heart attacks and strokes. Ketorolac is a very potent NSAID and is used for treating severe pain that normally would be managed with narcotics. Ketorolac causes ulcers more frequently than other NSAIDs and should not be used for more than five days. Celecoxib blocks COX-2 but has little effect on COX-1. Therefore, celecoxib is sub-classified as a selective COX-2 inhibitor, and it causes fewer ulcers and less bleeding than other NSAIDs. Commonly prescribed NSAIDs include aspirin, salsalate, celecoxib, diclofenac, etodolac, ibuprofen, indomethacin, ketoprofen, ketorolac, nabumetone, naproxen, oxaprozin, piroxicam, sulindac and tolmetin.

Neurological Diseases

Huntington's Disease and dyskinesias. Chorea is an abnormal involuntary movement disorder, one of a group of neurological disorders called dyskinesias, which are caused by overactivity of the neurotransmitter dopamine in the areas of the brain that control movement. Chorea is characterized by brief, irregular contractions that are not repetitive or rhythmic, but appear to flow from one muscle to the next. Chorea often occurs with athetosis, which adds twisting and writhing movements. Chorea is a primary feature of Huntington's disease, a progressive, hereditary movement disorder that appears in adults, but it may also occur in a variety of other conditions. Syndenham's chorea occurs in a small percentage (20 percent) of children and adolescents as a complication of rheumatic fever. Chorea can also be induced by drugs (levodopa, anti-convulsants, and anti-psychotics) metabolic and endocrine disorders, and vascular incidents. There is currently no standard course of treatment for chorea. Treatment depends on the type of chorea and the associated disease. Treatment for Huntington's disease is supportive, while treatment for Syndenham's chorea usually involves antibiotic drugs to treat the infection, followed by drug therapy to prevent recurrence. Adjusting medication dosages can treat drug-induced chorea. Metabolic and endocrine-related choreas are treated according to the cause(s) of symptoms.

Parkinson's Disease. Parkinson's disease (PD) belongs to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. PD usually affects people over the age of 50. Other symptoms may include depression and other emotional changes; difficulty in swallowing, chewing, and speaking; urinary problems or constipation; skin problems; and sleep disruptions. There are currently no blood or laboratory tests that have been proven to help in diagnosing sporadic PD. Therefore the diagnosis is based on medical history and a neurological examination. The disease can be difficult to diagnose accurately. There is no cure for PD, but a variety of medications are used to relieve symptoms. Patients are given levodopa combined with carbidopa. Carbidopa delays the conversion of levodopa into dopamine until it reaches the brain. Nerve cells can use levodopa to make dopamine and replenish the brain supply. Anticholinergics may help control tremor and rigidity. Other drugs, such as bromocriptine, pramipexole, and ropinirole, mimic the role of dopamine in the brain, causing the neurons to react as they would to dopamine. An antiviral drug, amantadine, also appears to reduce symptoms. Rasagiline can be used along with levodopa for patients with advanced PD or as a single-drug treatment for early PD. In some cases, surgery may be appropriate if the disease doesn't respond to drugs. A therapy called deep brain stimulation (DBS) has now been approved by the U.S. Food and Drug Administration. In DBS, electrodes are implanted into the brain and connected to a small electrical device called a pulse generator that can be externally programmed. DBS can reduce the need for levodopa and related drugs, which in turn decreases the involuntary movements called dyskinesias that are a common side effect of levodopa. It also helps to alleviate fluctuations of symptoms and to reduce tremors, slowness of movements, and gait problems. DBS requires careful programming of the stimulator device in order to work correctly.

Amyotrophic Lateral Sclerosis. Amyotrophic lateral sclerosis (ALS), sometimes called Lou Gehrig's disease or classical motor neuron disease, is a rapidly progressive, invariably fatal neurological disease that attacks the neurons responsible for controlling voluntary muscles. In ALS, both the upper motor neurons and the lower motor neurons degenerate or die, ceasing to send messages to muscles. Unable to function, the muscles gradually atrophy. Symptoms are usually first noticed in the arms and hands, legs, or swallowing muscles. Muscle weakness and atrophy occur on both sides of the body. Individuals with ALS lose their strength and the ability to move their arms and legs, and to hold the body upright. The disease does not affect a person's ability to see, smell, taste, hear, or recognize touch. Although the disease does not usually impair a person's mind or personality, several recent studies suggest that some people with ALS may develop cognitive problems involving word fluency, decision-making, and memory. The cause of ALS is not known. No cure has yet been found for ALS. The drug riluzole prolongs life by 2-3 months but does not relieve symptoms.

Multiple Sclerosis. Multiple sclerosis (MS) is a neurologic disease that can range from benign to completely disabling. MS results from an auto-immune response to nerve-insulating myelin. Such assaults may be linked to an unknown environmental trigger, perhaps a virus.

Most people experience their first symptoms of MS between the ages of 20 and 40; the initial symptom of MS is often blurred or double vision, red-green color distortion, or even blindness in one eye. Most MS patients experience muscle weakness in their extremities and difficulty with coordination and balance. These symptoms may be severe enough to impair walking or even standing. In the worst cases, MS can produce partial or complete paralysis. Most people with MS also exhibit paresthesias, transitory abnormal sensory feelings such as numbness, prickling, or “pins and needles” sensations. Some may also experience pain. Speech impediments, tremors, and dizziness are other frequent complaints. Occasionally, people with MS have hearing loss. Approximately half of all people with MS experience cognitive impairments such as difficulties with concentration, attention, memory, and poor judgment, but such symptoms are usually mild and are frequently overlooked. Depression is another common feature of MS. There is as yet no cure for MS. Three forms of beta interferon (Avonex, Betaseron, and Rebif) have now been approved by the Food and Drug Administration for treatment of relapsing-remitting MS. Beta interferon has been shown to reduce the number of exacerbations and may slow the progression of physical disability. When attacks do occur, they tend to be shorter and less severe. The FDA also has approved a synthetic form of myelin basic protein, called copolymer I (Copaxone), for the treatment of relapsing-remitting MS. An immunosuppressant treatment, Novantrone (mitoxantrone), is approved by the FDA for the treatment of advanced or chronic MS. The FDA has also approved dalfampridine (Ampyra) to improve walking in individuals with MS. While steroids do not affect the course of MS over time, they can reduce the duration and severity of attacks in some patients. Spasticity, which can occur either as a sustained stiffness caused by increased muscle tone or as spasms that come and go, is usually treated with muscle relaxants and tranquilizers such as baclofen, tizanidine, diazepam, clonazepam, and dantrolene. Other drugs that may reduce fatigue in some, but not all, patients include amantadine (Symmetrel), pemoline (Cylert), and the still-experimental drug aminopyridine. Although improvement of optic symptoms usually occurs even without treatment, a short course of treatment with intravenous methylprednisolone (Solu-Medrol) followed by treatment with oral steroids is sometimes used.

Alzheimer's Disease. Alzheimer's disease is an irreversible, progressive brain disease that slowly destroys memory and thinking skills. In most people with Alzheimer's, symptoms first appear after age 60. Estimates vary, but as many as 5.1 million Americans may have Alzheimer's disease. Patient's exhibit various brain abnormalities including amyloid plaques, neurofibrillary tangles, and neuronal loss. Four medications are approved by the U.S. Food and Drug Administration to treat Alzheimer's. Donepezil, rivastigmine and galantamine are used to treat mild to moderate Alzheimer's. Memantine is used to treat moderate to severe Alzheimer's. These drugs do not change the underlying disease process, are effective for some but not all people, and may help only for a limited time.

Schizophrenia. Schizophrenics display three broad categories of symptoms characterized as positive, negative and cognitive. Positive symptoms are psychotic behaviors including hallucinations, delusions, thought and movement disorders. Negative symptoms are associated with disruptions to normal behaviors. These symptoms include flat affect, lack of pleasure in everyday activities, lack of ability to begin and sustain planned activities, and speaking little, even when forced to interact as well as having neglect for basic personal hygiene. Cognitive symptoms include poor ability to understand information and use it to make decisions, trouble focusing or paying attention and problems with the ability to use information immediately after learning it. This neurologic disorder effects 1 percent of the general population, but it occurs in 10 percent of people who have a first-degree relative with the disorder. The risk is highest for an identical twin of a person with schizophrenia with a 40-65 percent chance of developing the disorder. No gene causes the disease by itself. Aberrant dopamine and glutamate transmission is believed to play a role in schizophrenia. Treatments include antipsychotic medications and various psychosocial treatments. Older antipsychotic medications include Chlorpromazine, Haloperidol, Perphenazine, Etrafon and Fluphenazine. New antipsychotic medications include clozapine which can cause agranulocytosis, requiring bi-weekly WBC count evaluation. Other atypical antipsychotics include Risperidone, Olanzapine, Quetiapine, Ziprasidone, Aripiprazole and Paliperidone. Side effects of many antipsychotics include drowsiness, dizziness when changing positions, blurred vision, rapid heartbeat, sensitivity to the sun, Skin rashes and menstrual problems for women. Atypical antipsychotic medications can cause major weight gain and changes in a person's metabolism. This may increase a person's risk of getting diabetes and high cholesterol. Typical antipsychotic medications can cause side effects related to physical movement, such as rigidity, persistent muscle spasms, tremors and restlessness. Long-term use of typical antipsychotic medications may lead to a condition called tardive dyskinesia (TD). TD causes uncontrolled, and in some cases permanent, involuntary muscle movements.

### Additional Description of the Invention

The present invention relates to methods and compositions for the treatment of any gene that is desirable to modulate expression of. This includes but is not limited to cancers. In the next sections will will describe both cancer and non-cancer targets and then in the section immediately following those selected cancer and non-cancer targets we will present over 40 High Value Targets, both cancer and noncancer, with sequence information, and some of these examples will have data with detailed information about our techniques and methods as well as our surprising results.

Cancer Targets

In some embodiments, the present invention provides oligonucleotide-based therapeutics for the inhibition of oncogenes involved in a variety of cancers. The present invention is not limited to the treatment of cancer or any particular cancer. Any cancer can be targeted, including, but not limited to, breast cancers. The present invention is also not limited to the targeting of cancers or oncogenes. The methods and compositions of the present invention are suitable for use with any gene that it is desirable to inhibit the expression of (e.g., for therapeutic or research uses. Specific gene targets that have been optimally identified as susceptible to the DNAi therapeutic approach are described below.

Oncogene Targets such as,

In some embodiments, the present invention provides DNAi inhibitors of oncogenes. The present invention is not limited to the inhibition of a particular oncogene. Indeed, the present invention encompasses DNAi inhibitors to any number of oncogenes including, but not limited to, those disclosed herein.

Combination Therapies with Cancer Targets

In some embodiments, the compositions of the present invention are provided in combination with existing therapies. In other embodiments, two or more compounds of the present invention are provided in combination. In some embodiments, the compounds of the present invention are provided in combination with known cancer chemotherapy agents. The present invention is not limited to a particular chemotherapy agent.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, |
| | | Inc., San Diego CA |
| Allopurinol | Zyloprim | GlaxoSmithKline, |
| (1,5-dihydro-4H-pyrazolo[3,4-d]pyrimidin-4-one | | Research Triangle |
| monosodium salt) | | Park, NC |
| Altretamine | Hexalen | US Bioscience, |
| (N,N,N′,N′,N″,N″,-hexamethyl-1,3,5-triazine-2,4,6- | | West |
| triamine) | | Conshohocken, PA |
| Amifostine | Ethyol | US Bioscience |
| (ethanethiol, 2-[(3-aminopropyl)amino]-, dihydrogen | | |
| phosphate (ester)) | | |
| Anastrozole | Arimidex | AstraZeneca |
| (1,3-Benzenediacetonitrile,a,a,a′,a′-tetramethyl-5- | | Pharmaceuticals, |
| (1H-1,2,4-triazol-1-ylmethyl)) | | LP, Wilmington, |
| | | DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse |
| | | Station, NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| Bexarotene gel | Targretin | Ligand |
| | | Pharmaceuticals |
| Bleomycin | Blenoxane | Bristol-Myers |
| (cytotoxic glycopeptide antibiotics produced by | | Squibb Co., NY, |
| Streptomyces verticillus; bleomycin A2 and | | NY |
| bleomycin B2) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers |
| (platinum, diammine [1,1- | | Squibb |
| cyclobutanedicarboxylato(2-)-0,0′]-,(SP-4-2)) | | |
| Carmustine | BCNU, | Bristol-Myers |
| (1,3-bis(2-chloroethyl)-1-nitrosourea) | BiCNU | Squibb |
| Carmustine with Polifeprosan 20 Implant | Gliadel | Guilford |
| | Wafer | Pharmaceuticals, |
| | | Inc., Baltimore, |
| | | MD |
| Celecoxib | Celebrex | Searle |
| (as 4-[5-(4-methylphenyl)-3-(trifluoromethyl)-1H- | | Pharmaceuticals, |
| pyrazol-1-yl] benzenesulfonamide) | | England |
| Chlorambucil | Leukeran | GlaxoSmithKline |
| (4-[bis(2chlorethyl)amino]benzenebutanoic acid) | | |
| Cisplatin | Platinol | Bristol-Myers |
| (PtC12H6N2) | | Squibb |
| Cladribine | Leustatin, 2- | R. W. Johnson |
| (2-chloro-2′-deoxy-b-D-adenosine) | CdA | Pharmaceutical |
| | | Research Institute, |
| | | NJ |
| Cyclophosphamide | Cytoxan, | Bristol-Myers |
| (2-[bis(2-chloroethyl)amino] tetrahydro-2H-13,2- | Neosar | Squibb |
| oxazaphosphorine 2-oxide monohydrate) | | |
| Cytarabine | Cytosar-U | Pharmacia & |
| (1-b-D-Arabinofuranosylcytosine, C9H13N3O5) | | Upjohn Company |
| Cytarabine liposomal | DepoCyt | Skye |
| | | Pharmaceuticals, |
| | | Inc., San Diego, |
| | | CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C62H86N12O16) | | |
| Darbepoetin alfa | Aranesp | Amgen, Inc., |
| (recombinant peptide) | | Thousand Oaks, |
| | | CA |
| daunorubicin liposomal | DanuoXome | Nexstar |
| ((8S-cis)-8-acetyl-10-[(3-amino-2,3,6-trideoxy-á-L- | | Pharmaceuticals, |
| lyxo-hexopyranosyl)oxy]-7,8,9,10-tetrahydro-6,8,11- | | Inc., Boulder, CO |
| trihydroxy-1-methoxy-5,12-naphthacenedione | | |
| hydrochloride) | | |
| Daunorubicin HCl, daunomycin | Cerubidine | Wyeth Ayerst, |
| ((1S,3S)-3-Acetyl-1,2,3,4,6,11-hexahydro-3,5,12- | | Madison, NJ |
| trihydroxy-10-methoxy-6,11-dioxo-1-naphthacenyl | | |
| 3-amino-2,3,6-trideoxy-(alpha)-L-lyxo- | | |
| hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & |
| ((S)-4,4′-(1-methyl-1,2-ethanediyl)bis-2,6- | | Upjohn Company |
| piperazinedione) | | |
| Docetaxel | Taxotere | Aventis |
| ((2R,3S)-N-carboxy-3-phenylisoserine, N-tert-butyl | | Pharmaceuticals, |
| ester, 13-ester with 5b-20-epoxy-12a,4,7b,10b,13a- | | Inc., Bridgewater, |
| hexahydroxytax-11-en-9-one 4-acetate 2-benzoate, | | NJ |
| trihydrate) | | |
| Doxorubicin HCl | Adriamycin, | Pharmacia & |
| (8S,10S)-10-[(3-amino-2,3,6-trideoxy-a-L-lyxo- | Rubex | Upjohn Company |
| hexopyranosyl)oxy]-8-glycolyl-7,8,9,10-tetrahydro- | | |
| 6,8,11-trihydroxy-1-methoxy-5,12- | | |
| naphthacenedione hydrochloride) | | |
| doxorubicin | Adriamycin | Pharmacia & |
| | PFS | Upjohn Company |
| | Intravenous | |
| | injection | |
| doxorubicin liposomal | Doxil | Sequus |
| | | Pharmaceuticals, |
| | | Inc., Menlo park, |
| | | CA |
| dromostanolone propionate | Dromostanolone | Eli Lilly & |
| (17b-Hydroxy-2a-methyl-5a-androstan-3-one | | Company, |
| propionate) | | Indianapolis, IN |
| dromostanolone propionate | Masterone | Syntex, Corp., Palo |
| | injection | Alto, CA |
| Elliott′s B Solution | Elliott′s B | Orphan Medical, |
| | Solution | Inc |
| Epirubicin | Ellence | Pharmacia & |
| ((8S-cis)-10-[(3-amino-2,3,6-trideoxy-a-L-arabino- | | Upjohn Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & |
| (estra-1,3,5(10)-triene-3,17-diol(17(beta))-, 3-[bis(2- | | Upjohn Company |
| chloroethyl)carbamate] 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers |
| (4′-Demethylepipodophyllotoxin9-[4,6-O-(R)- | | Squibb |
| ethylidene-(beta)-D-glucopyranoside], 4′- | | |
| (dihydrogen phosphate)) | | |
| etoposide, VP-16 | Vepesid | Bristol-Myers |
| (4′-demethylepipodophyllotoxin 9-[4,6-0-(R)- | | Squibb |
| ethylidene-(beta)-D-glucopyranoside]) | | |
| Exemestane | Aromasin | Pharmacia & |
| (6-methylenandrosta-1,4-diene-3,17-dione) | | Upjohn Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex |
| (fluorinated nucleotide analog of the antiviral agent | | Laboratories, Inc., |
| vidarabine, 9-b-D-arabinofuranosyladenine (ara-A)) | | Cedar Knolls, NJ |
| Fluorouracil, 5-FU | Adrucil | ICN |
| (5-fluoro-2,4(1H,3H)-pyrimidinedione) | | Pharmaceuticals, |
| | | Inc., Humacao, |
| | | Puerto Rico |
| Fulvestrant | Faslodex | IPR |
| (7-alpha-[9-(4,4,5,5,5-penta fluoropentylsulphinyl) | | Pharmaceuticals, |
| nonyl]estra-1,3,5-(10)-triene-3,17-beta-diol) | | Guayama, Puerto |
| | | Rico |
| Gemcitabine | Gemzar | Eli Lilly |
| (2′-deoxy-2′,2′-difluorocytidine monohydrochloride | | |
| (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| (acetate salt of [D-Ser(But)6,Azgly10]LHRH; pyro- | Implant | Pharmaceuticals |
| Glu-His-Trp-Ser-Tyr-D-Ser(But)-Leu-Arg-Pro- | | |
| Azgly-NH2 acetate [C59H84N18O14•(C2H4O2)x | | |
| Hydroxyurea | Hydrea | Bristol-Myers |
| | | Squibb |
| Ibritumomab Tiuxetan | Zevalin | Biogen IDEC, Inc., |
| (immunoconjugate resulting from a thiourea covalent | | Cambridge MA |
| bond between the monoclonal antibody Ibritumomab | | |
| and the linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & |
| (5,12-Naphthacenedione, 9-acetyl-7-[(3-amino- | | Upjohn Company |
| 2,3,6-trideoxy-(alpha)-L-lyxo-hexopyranosyl)oxy]- | | |
| 7,8,9,10-tetrahydro-6,9,11-trihydroxyhydrochloride, | | |
| (7S-cis)) | | |
| Ifosfamide | IFEX | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| Imatinib Mesilate | Gleevec | Novartis AG, |
| (4-[(4-Methyl-1-piperazinyl)methyl]-N-[4-methyl-3- | | Basel, Switzerland |
| [[4-(3 -pyridinyl)-2-pyrimidinyl] amino]- | | |
| phenyl]benzamide methanesulfonate) | | |
| Interferon alfa-2a | Roferon-A | Hoffmann-La |
| (recombinant peptide) | | Roche, Inc., Nutley, |
| | | NJ |
| Interferon alfa-2b | Intron A | Schering AG, |
| (recombinant peptide) | (Lyophilized | Berlin, Germany |
| | Betaseron) | |
| Irinotecan HCl | Camptosar | Pharmacia & |
| ((4S)-4,11-diethyl-4-hydroxy-9-[(4-piperi- | | Upjohn Company |
| dinopiperidino)carbonyloxy]-1H-pyrano[3′,4′:6,7] | | |
| indolizino[1,2-b] quinoline-3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl-1,4,5,6,7,8 | Leucovorin | Seattle, WA |
| hexahydro4oxo6-pteridinyl)methyl] amino]benzoyl] , | | |
| calcium salt (1:1)) | | |
| Levamisole HCl | Ergamisol | Janssen Research |
| ((−)-(S)-2,3,5,6-tetrahydro-6-phenylimidazo [2,1-b] | | Foundation, |
| thiazole monohydrochloride C11H12N2S•HCl) | | Titusville, NJ |
| Lomustine | CeeNU | Bristol-Myers |
| (1-(2-chloro-ethyl)-3-cyclohexyl-1-nitrosourea) | | Squibb |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers |
| 17α(acetyloxy)-6-methylpregna-4,6-diene-3,20- | | Squibb |
| dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle |
| (N-[4-[[(2,4-diamino-6- | | Laboratories |
| pteridinyl)methyl]methylamino]benzoyl]-L-glutamic | | |
| acid) | | |
| Methoxsalen (9-methoxy-7H-furo[3,2-g][1]- | Uvadex | Therakos, Inc., |
| benzopyran-7-one) | | Way Exton, Pa |
| Mitomycin C | Mutamycin | Bristol-Myers |
| | | Squibb |
| Mitomycin C | Mitozytrex | SuperGen, Inc., |
| | | Dublin, CA |
| Mitotane | Lysodren | Bristol-Myers |
| (1,1-dichloro-2-(o-chlorophenyl)-2-(p-chlorophenyl) | | Squibb |
| ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer |
| | | Ingelheim Pharma |
| | | KG, Germany |
| Oprelvekin | Neumega | Genetics Institute, |
| (IL-11) | | Inc., Alexandria, |
| | | VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers |
| (5β,20-Epoxy-1,2a,4,7β,10β,13a-hexahydroxytax- | | Squibb |
| 11-en-9-one 4,10-diacetate 2-benzoate 13-ester with | | |
| (2R,3S)-N-benzoyl-3-phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) 11- | (Pegademase | Pharmaceuticals, |
| 17-adenosine deaminase) | Bovine) | Inc., Bridgewater, |
| | | NJ |
| Pegaspargase | Oncaspar | Enzon |
| (monomethoxypolyethylene glycol succinimidyl L- | | |
| asparaginase) | | |
| Pegfilgrastim | Neulasta | Amgen, Inc |
| (covalent conjugate of recombinant methionyl human | | |
| G-CSF (Filgrastim) and monomethoxypolyethylene | | |
| glycol) | | |
| Pentostatin | Nipent | Parke-Davis |
| | | Pharmaceutical Co., |
| | | Rockville, MD |
| Pipobroman | Vercyte | Abbott |
| | | Laboratories, |
| | | Abbott Park, IL |
| Plicamycin, Mithramycin | Mithracin | Pfizer, Inc., NY, |
| (antibiotic produced by Streptomyces plicatus) | | NY |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, |
| monohydrochloride) | | Inc., Gaithersburg, |
| | | MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San |
| | | Francisco, CA |
| Sargramostim | Prokine | Immunex Corp |
| (recombinant peptide) | | |
| Streptozocin | Zanosar | Pharmacia & |
| (streptozocin 2-deoxy-2- | | Upjohn Company |
| [[(methylnitrosoamino)carbonyl]amino]-a(and b)- | | |
| D-glucopyranose and 220 mg citric acid anhydrous) | | |
| Talc | Sclerosol | Bryan, Corp., |
| (Mg3Si4O10 (OH)2) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| Teniposide, VM-26 | Vumon | Bristol-Myers |
| (4′-demethylepipodophyllotoxin 9-[4,6-0-(R)-2- | | Squibb |
| thenylidene-(beta)-D-glucopyranoside]) | | |
| Testolactone | Teslac | Bristol-Myers |
| (13-hydroxy-3-oxo-13,17-secoandrosta-1,4-dien-17- | | Squibb |
| oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex |
| (Aziridine,1,1′,1″-phosphinothioylidynetris-, or Tris | | Corporation |
| (1-aziridinyl) phosphine sulfide) | | |
| Topotecan HCl | Hycamtin | GlaxoSmithKline |
| ((S)-10-[(dimethylamino)methyl]-4-ethyl-4,9- | | |
| dihydroxy-1H-pyrano[3′,4′:6,7] indolizino [1,2-b] | | |
| quinoline-3,14-(4H,12H)-dione monohydrochloride) | | |
| Toremifene | Fareston | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, |
| | | NJ |
| Tositumomab, I 131 Tositumomab | Bexxar | Corixa Corp., |
| (recombinant murine immunotherapeutic monoclonal | | Seattle, WA |
| IgG2a lambda anti-CD20 antibody (I 131 is a | | |
| radioimmunotherapeutic antibody)) | | |
| Trastuzumab | Herceptin | Genentech, Inc |
| (recombinant monoclonal IgG1 kappa anti-HER2 | | |
| antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6-trideoxy- | | |
| 3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C46H56N4O10•H2SO4) | | |
| Vincristine | Oncovin | Eli Lilly |
| (C46H56N4O10•H2SO4) | | |
| Vinorelbine | Navelbine | GlaxoSmithKline |
| (3′,4′-didehydro-4′-deoxy-C′-norvincaleukoblastine | | |
| [R-(R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Other identified cancer combination therapies include the following: PI3K inhibitors (CAL101), Bruton Kinase inhibitor (PCI-32765), and BCL-6 inhibitor. This document describes the targets and associated therapy for these identified cancers as being particularly susceptible to treatment with combination therapies. Targets

The present invention is not limited to the cancer and non-cancer targets listed above commonly found in humans. The present invention can also be applied both to other cancer targets (also referred to as oncogenes) (and where such cancer targets may also be involved in other disease such as inflammation, neurological, metabolic, cardiovascular, etc.) and to non-cancer target such as Cardiovascular/Metabolic Disease, Eye Disease, Infectious Disease, Inflammation, Neurological Disease, Rare Disease, and Stem Cells. Examples of specific genes are included in Table 2, but are not limited to those described in Table. Additional targets are not listed but can be found in the key proliferation pathways such as MAPK, PI3K, MEK, etc. The present invention can also apply to disease and growth targets for plant genome and animal genomes.

TABLE 2
Cancer and non-cancer targets
DNAi Disease, Gene, and Cell System Targets

| ID | Disease Area | Target |
|---|---|---|
| 1 | Cancer | 2-dG |
| 2 | Cancer | 4-1BB |
| 3 | Cancer | ABCB1 |
| 4 | Cancer | ABL |
| 5 | Cancer | ABL1/BCR |
| 6 | Cancer | Act-1 |
| 7 | Cancer | ADAM12 |
| 8 | Cancer | ADAM7 |
| 9 | Cancer | ADAMTS4 |
| 10 | Cancer | ADAMTS5 |
| 11 | Cancer | AFP (Alpha-fetoprotein) |
| 12 | Cancer | AKT |
| 13 | Cancer | AKT1 |
| 14 | Cancer | AKT2 |
| 15 | Cancer | AKT3 |
| 16 | Cancer | AldoA |
| 17 | Cancer | ALK |
| 18 | Cancer | ALK/NPM1 |
| 19 | Cancer | AMI1 |
| 20 | Cancer | AML1/ETO |
| 21 | Cancer | Androgen Receptor (AR) |
| 22 | Cancer | Angiopoeitin (ANG) |
| 23 | Cancer | ANGPT2 (ANG-2) |
| 24 | Cancer | APC |
| 25 | Cancer | ARAF |
| 26 | Cancer | AR |
| 27 | Cancer | AREG |
| 28 | Cancer | ARF6 |
| 29 | Cancer | ARNT |
| 30 | Cancer | Aromatase Inhibitors (Ais) |
| 31 | Cancer | ASXL1 |
| 32 | Cancer | ATM |
| 33 | Cancer | ATRX |
| 34 | Cancer | AXIN1 |
| 35 | Cancer | AXL |
| 36 | Cancer | B7H3 |
| 37 | Cancer | BAX |
| 38 | Cancer | BBC3 |
| 39 | Cancer | BCBL |
| 40 | Cancer | BCL1 |
| 41 | Cancer | BCL2 |
| 42 | Cancer | BCL2L1 (BCLXL) |
| 43 | Cancer | BCL2L 11 |
| 44 | Cancer | BCL3 |
| 45 | Cancer | BCL6 |
| 46 | Cancer | BCR/ABL |
| 47 | Cancer | BDNF |
| 48 | Cancer | Beclin-1 |
| 49 | Cancer | Beta catenin |
| 50 | Cancer | BIRC2 (c-IAP1) |
| 51 | Cancer | BIRC3 (c-IAP2) |
| 52 | Cancer | BIRC4 |
| 53 | Cancer | BIRC5 |
| 54 | Cancer | BMI1 |
| 55 | Cancer | BMP10 |
| 56 | Cancer | BRAF |
| 57 | Cancer | BRCA1 |
| 58 | Cancer | BRCA2 |
| 59 | Cancer | BRD3 |
| 60 | Cancer | BTK |
| 61 | Cancer | BTLA |
| 62 | Cancer | C/EBPalpha |
| 63 | Cancer | C5B-9 |
| 64 | Cancer | CANT1 |
| 65 | Cancer | CASP2 |
| 66 | Cancer | CASP3 |
| 67 | Cancer | CASP8 |
| 68 | Cancer | CBFA2T3 |
| 69 | Cancer | CBFB |
| 70 | Cancer | CBL |
| 71 | Cancer | CBLB |
| 72 | Cancer | CBLC |
| 73 | Cancer | CCND1 |
| 74 | Cancer | CCND3 |
| 75 | Cancer | CCKBR |
| 76 | Cancer | CCNA1 |
| 77 | Cancer | CCNB1 |
| 78 | Cancer | CD133 |
| 79 | Cancer | CD19 |
| 80 | Cancer | CD20 |
| 81 | Cancer | CD24 |
| 82 | Cancer | CD30 |
| 83 | Cancer | CD33 |
| 84 | Cancer | CD37 |
| 85 | Cancer | CD38 |
| 86 | Cancer | CD4 |
| 87 | Cancer | CD-40 |
| 88 | Cancer | CD40LG |
| 89 | Cancer | CD44 |
| 90 | Cancer | CD-52 |
| 91 | Cancer | CD74 |
| 92 | Cancer | CD80 |
| 93 | Cancer | CDC42 |
| 94 | Cancer | CDC25A |
| 95 | Cancer | CDC25B |
| 96 | Cancer | CDK2 |
| 97 | Cancer | CDK4 |
| 98 | Cancer | CDK4 |
| 99 | Cancer | CDK6 |
| 100 | Cancer | CDK7 |
| 101 | Cancer | CDKN1A |
| 102 | Cancer | CDKN1C |
| 103 | Cancer | CDKN2A |
| 104 | Cancer | CDKN2B |
| 105 | Cancer | CDKN2C |
| 106 | Cancer | c-fos |
| 107 | Cancer | CHEK1 |
| 108 | Cancer | CHEK2 |
| 109 | Cancer | CHMP5 |
| 110 | Cancer | c-ki-RAS |
| 111 | Cancer | CKIT |
| 112 | Cancer | CLTC |
| 113 | Cancer | Clusterin |
| 114 | Cancer | CMET |
| 115 | Cancer | COL6A3 |
| 116 | Cancer | CPK |
| 117 | Cancer | CRAF |
| 118 | Cancer | CRB |
| 119 | Cancer | CRBN |
| 120 | Cancer | CRCT1/TORC1 |
| 121 | Cancer | CRK |
| 122 | Cancer | CRK-II |
| 123 | Cancer | CRM1 |
| 124 | Cancer | Crry |
| 125 | Cancer | CSF1R/FMS |
| 126 | Cancer | CSN5 |
| 127 | Cancer | c-SRC |
| 128 | Cancer | CATG1B |
| 129 | Cancer | CTAG2 |
| 130 | Cancer | CTCF |
| 131 | Cancer | CTFG |
| 132 | Cancer | CTLA-4 |
| 133 | Cancer | CTNNB1 |
| 134 | Cancer | CTSB |
| 135 | Cancer | CTSL2 |
| 136 | Cancer | CX3CL1 |
| 137 | Cancer | CXCL12 |
| 138 | Cancer | CYCS |
| 139 | Cancer | CYLD |
| 140 | Cancer | CYR61 |
| 141 | Cancer | DAL1L |
| 142 | Cancer | DAPK1 |
| 143 | Cancer | DBL |
| 144 | Cancer | DCC |
| 145 | Cancer | DCN |
| 146 | Cancer | DCL1 |
| 147 | Cancer | DDB2 |
| 148 | Cancer | DDOST |
| 149 | Cancer | DDX6 |
| 150 | Cancer | DEK |
| 151 | Cancer | DHFR |
| 152 | Cancer | DIABLO |
| 153 | Cancer | DKK1 |
| 154 | Cancer | DNMT1 |
| 155 | Cancer | DNMT(3A) |
| 156 | Cancer | DNMT(3B) |
| 157 | Cancer | DOT1L |
| 158 | Cancer | DPC4/SMAD4 |
| 159 | Cancer | DPP-IV |
| 160 | Cancer | E2F |
| 161 | Cancer | E2F1 |
| 162 | Cancer | E2F1/RBAP |
| 163 | Cancer | E2F3 |
| 164 | Cancer | EBF1 |
| 165 | Cancer | E-CAD |
| 166 | Cancer | Ecadherin |
| 167 | Cancer | EGF |
| 168 | Cancer | EGFL7 |
| 169 | Cancer | EGFR |
| 170 | Cancer | EGFR/ERBB-1 |
| 171 | Cancer | EGFR/HER1 |
| 172 | Cancer | EIF4A2 |
| 173 | Cancer | eIF-4E |
| 174 | Cancer | ELK1 |
| 175 | Cancer | ELK3 |
| 176 | Cancer | EP300 |
| 177 | Cancer | EPCAM |
| 178 | Cancer | EPH |
| 179 | Cancer | EPHA1 |
| 180 | Cancer | EPHA3 |
| 181 | Cancer | ER |
| 182 | Cancer | ERBB-3 |
| 183 | Cancer | ERG |
| 184 | Cancer | ERK |
| 185 | Cancer | e-selectin (SELE) |
| 186 | Cancer | Estrogen Receptor (ESR1) |
| 187 | Cancer | ETS1 |
| 188 | Cancer | ETS2 |
| 189 | Cancer | ETV6 (TEL) |
| 190 | Cancer | EZH2 |
| 191 | Cancer | FAK |
| 192 | Cancer | FANCA |
| 193 | Cancer | FAP |
| 194 | Cancer | FAS |
| 195 | Cancer | FASLG |
| 196 | Cancer | FBXW7 |
| 197 | Cancer | FER |
| 198 | Cancer | FGF6 |
| 199 | Cancer | FGF7 |
| 200 | Cancer | FGFR-TACC fusion protein |
| 201 | Cancer | FGFR1 |
| 202 | Cancer | FGFR2 |
| 203 | Cancer | FGR |
| 204 | Cancer | Fibroblast growth factor |
| | | (FGF), 1, 2, |
| 205 | Cancer | FLI1/ERGB2 |
| 206 | Cancer | FLI1/ERGB2 |
| 207 | Cancer | FLT1 (VEGFR1) |
| 208 | Cancer | FLT3 |
| 209 | Cancer | FLT4 |
| 210 | Cancer | FMS |
| 211 | Cancer | FOLH1 (PSMA) |
| 212 | Cancer | FOS |
| 213 | Cancer | FOSL1 |
| 214 | Cancer | FOSL2 |
| 215 | Cancer | FOXE1 |
| 216 | Cancer | FPS/FES |
| 217 | Cancer | FRA1 |
| 218 | Cancer | FRA2 |
| 219 | Cancer | FST |
| 220 | Cancer | FT3 |
| 221 | Cancer | FUBP1 |
| 222 | Cancer | Furin |
| 223 | Cancer | FYN |
| 224 | Cancer | GADD45A |
| 225 | Cancer | GADD45B |
| 226 | Cancer | GATA4 |
| 227 | Cancer | GDF2 |
| 228 | Cancer | GIP |
| 229 | Cancer | GLI |
| 230 | Cancer | GNA11 |
| 231 | Cancer | GHAQ |
| 232 | Cancer | GNAS1 |
| 233 | Cancer | GNAS2 |
| 234 | Cancer | GRB-2 |
| 235 | Cancer | GRN |
| 236 | Cancer | GSK3A |
| 237 | Cancer | GSP |
| 238 | Cancer | GST-Pi |
| 239 | Cancer | HAT1 |
| 240 | Cancer | HCK |
| 241 | Cancer | HDAC1 |
| 242 | Cancer | HDAC10 |
| 243 | Cancer | HDAC11 |
| 244 | Cancer | HDAC2 |
| 245 | Cancer | HDAC4 |
| 246 | Cancer | HDAC5 |
| 247 | Cancer | HDAC6 |
| 248 | Cancer | HDAC7 |
| 249 | Cancer | HDAC8 |
| 250 | Cancer | HDAC9 |
| 251 | Cancer | Hedgehog |
| 252 | Cancer | HEK |
| 253 | Cancer | Her-2 |
| 254 | Cancer | HER2/ERBB2 |
| 255 | Cancer | HER3 |
| 256 | Cancer | HER3/ERBB-2 |
| 257 | Cancer | HER4 |
| 258 | Cancer | HER4/ERBB-4 |
| 259 | Cancer | HIF1A |
| 260 | Cancer | HIF2A |
| 261 | Cancer | HIF-1beta |
| 262 | Cancer | HIND |
| 263 | Cancer | hMOF |
| 264 | Cancer | HMGA1 |
| 265 | Cancer | HMGB1 |
| 266 | Cancer | HMTs |
| 267 | Cancer | HOX11 |
| 268 | Cancer | HOXA7 |
| 269 | Cancer | HOXD10 |
| 270 | Cancer | HPC1 |
| 271 | Cancer | HRAS (c-ha-ras) |
| 272 | Cancer | HRX/MLLT1 |
| 273 | Cancer | HRX/MLLT2 |
| 274 | Cancer | Hsp27 |
| 275 | Cancer | Hsp70 (HSPBP1) |
| 276 | Cancer | HSP-90 |
| 277 | Cancer | HST |
| 278 | Cancer | HST2 |
| 279 | Cancer | HSTF1 |
| 280 | Cancer | HTRA3 |
| 281 | Cancer | IDH (2H) |
| 282 | Cancer | IDH1 |
| 283 | Cancer | IDH2 |
| 284 | Cancer | IDO |
| 285 | Cancer | IFNA1 |
| 286 | Cancer | IGF1 |
| 287 | Cancer | IGF1R |
| 288 | Cancer | IGF2 |
| 289 | Cancer | IGFBP2 |
| 290 | Cancer | IGFBP5 |
| 291 | Cancer | IL-17 |
| 292 | Cancer | IL-23 |
| 293 | Cancer | IL3 |
| 294 | Cancer | IL3RA |
| 295 | Cancer | IL4RA |
| 296 | Cancer | IL-6 |
| 297 | Cancer | IL8 |
| 298 | Cancer | ING4 |
| 299 | Cancer | INK4A (p16) |
| 300 | Cancer | INK4B |
| 301 | Cancer | INT-1 |
| 302 | Cancer | INT1/WNT1 |
| 303 | Cancer | INT2 |
| 304 | Cancer | IRF1 |
| 305 | Cancer | IRP2 |
| 306 | Cancer | ITGB1 |
| 307 | Cancer | JAG1 |
| 308 | Cancer | JAK1 |
| 309 | Cancer | JAK2 |
| 310 | Cancer | JAK3 |
| 311 | Cancer | JUN |
| 312 | Cancer | JUNB |
| 313 | Cancer | JUND |
| 314 | Cancer | KAT6A |
| 315 | Cancer | KDM6A |
| 316 | Cancer | KIF5B |
| 317 | Cancer | KIP2 |
| 318 | Cancer | KIT |
| 319 | Cancer | KITLG |
| 320 | Cancer | KRAS |
| 321 | Cancer | KRAS2 |
| 322 | Cancer | KRAS2A |
| 323 | Cancer | KRAS2B |
| 324 | Cancer | KS3 |
| 325 | Cancer | K-SAM |
| 326 | Cancer | KSP |
| 327 | Cancer | LAG3 |
| 328 | Cancer | LATS1 |
| 329 | Cancer | LBC |
| 330 | Cancer | LCK |
| 331 | Cancer | LEF1 |
| 332 | Cancer | LET-7 |
| 333 | Cancer | LIMK1 |
| 334 | Cancer | LMO-1 |
| 335 | Cancer | LMO-2 |
| 336 | Cancer | L-MYC |
| 337 | Cancer | LSD1 |
| 338 | Cancer | 1-selectin |
| 339 | Cancer | LYL1 |
| 340 | Cancer | LYN |
| 341 | Cancer | LYT-10 |
| 342 | Cancer | MADH4 |
| 343 | Cancer | MALT1 |
| 344 | Cancer | MAP2K1 |
| 345 | Cancer | MAP3K3 |
| 346 | Cancer | MAP3K10 |
| 347 | Cancer | MAP3K11 |
| 348 | Cancer | MAP3K14 |
| 349 | Cancer | MAP4K4 |
| 350 | Cancer | MAPK |
| 351 | Cancer | MAPK1 |
| 352 | Cancer | MAPK9 |
| 353 | Cancer | MAS |
| 354 | Cancer | MAS1 |
| 355 | Cancer | MASXL1 |
| 356 | Cancer | MTA2 |
| 357 | Cancer | MAX |
| 358 | Cancer | MCC |
| 359 | Cancer | MCF2 |
| 360 | Cancer | MCL1 |
| 361 | Cancer | MDM2 |
| 362 | Cancer | MDM4 |
| 363 | Cancer | MEF2C |
| 364 | Cancer | MEK1 |
| 365 | Cancer | MEK2 |
| 366 | Cancer | MEN1 |
| 367 | Cancer | MEN2 |
| 368 | Cancer | MET |
| 369 | Cancer | Metabolites |
| 370 | Cancer | Methyltransferase |
| 371 | Cancer | MGLL |
| 372 | Cancer | MGMT |
| 373 | Cancer | MIDHI? |
| 374 | Cancer | MLH1 |
| 375 | Cancer | MLL |
| 376 | Cancer | MLLT1/MLL |
| 377 | Cancer | MLLT2/HRX |
| 378 | Cancer | MLM |
| 379 | Cancer | MMP |
| 380 | Cancer | MMP1 |
| 381 | Cancer | MMP13 |
| 382 | Cancer | MMP2 |
| 383 | Cancer | MMP9 |
| 384 | Cancer | MNK |
| 385 | Cancer | MOS |
| 386 | Cancer | MSH2 |
| 387 | Cancer | MSH6 |
| 388 | Cancer | MTG8/RUNX1 |
| 389 | Cancer | MTOR |
| 390 | Cancer | MTORC2 |
| 391 | Cancer | MUC1 |
| 392 | Cancer | MYB |
| 393 | Cancer | MYBA |
| 394 | Cancer | MYBB |
| 395 | Cancer | MYC (CMYC) |
| 396 | Cancer | MYCC/MCYN |
| 397 | Cancer | MYCL1 |
| 398 | Cancer | MYCLK1 |
| 399 | Cancer | MYCN |
| 400 | Cancer | MYH11//CBFB |
| 401 | Cancer | MXD1 |
| 402 | Cancer | MXI1 |
| 403 | Cancer | NAFT4 |
| 404 | Cancer | NAFT5 |
| 405 | Cancer | NAIP |
| 406 | Cancer | Nampt |
| 407 | Cancer | NANOG |
| 408 | Cancer | NCL (nucleolin) |
| 409 | Cancer | NCOA6 |
| 410 | Cancer | NCOR2 |
| 411 | Cancer | NDN |
| 412 | Cancer | NF1 |
| 413 | Cancer | NF2 |
| 414 | Cancer | NFI-A |
| 415 | Cancer | NFKB |
| 416 | Cancer | NFKB1 |
| 417 | Cancer | NFKB2 |
| 418 | Cancer | NGFR |
| 419 | Cancer | NME1 |
| 420 | Cancer | N-MYC |
| 421 | Cancer | NOS2A |
| 422 | Cancer | NOTCH1 |
| 423 | Cancer | NPM1 |
| 424 | Cancer | NPM1/ALK |
| 425 | Cancer | NPTX1 |
| 426 | Cancer | NR3C1 |
| 427 | Cancer | NRAS |
| 428 | Cancer | NRG/REL |
| 429 | Cancer | NSD3 |
| 430 | Cancer | NTRK1 |
| 431 | Cancer | NUAK1 |
| 432 | Cancer | NUP214 |
| 433 | Cancer | OSM |
| 434 | Cancer | OST |
| 435 | Cancer | OX40/CD134 |
| 436 | Cancer | P2Y12 |
| 437 | Cancer | P53 (TP53) |
| 438 | Cancer | P57/KIP2 |
| 439 | Cancer | p85beta |
| 440 | Cancer | PACE4 |
| 441 | Cancer | PAK4 |
| 442 | Cancer | PALB2 |
| 443 | Cancer | PARP |
| 444 | Cancer | PARP1 |
| 445 | Cancer | PARP2 |
| 446 | Cancer | PAX-5 |
| 447 | Cancer | PBRM1 |
| 448 | Cancer | PBX1/TCF3 |
| 449 | Cancer | PD1 |
| 450 | Cancer | PDCD4 |
| 451 | Cancer | PDFGR/FILP1L1-PDFGRa |
| 452 | Cancer | PDGF |
| 453 | Cancer | PDGFB |
| 454 | Cancer | PDGFR |
| 455 | Cancer | PDGFRA |
| 456 | Cancer | PDL1/2 |
| 457 | Cancer | Pfk |
| 458 | Cancer | Pfkfb3 |
| 459 | Cancer | PGAM1 |
| 460 | Cancer | PHDGH |
| 461 | Cancer | PHF6 |
| 462 | Cancer | PI3K |
| 463 | Cancer | PIGF |
| 464 | Cancer | PIM1 |
| 465 | Cancer | PKCα |
| 466 | Cancer | Pkm2 |
| 467 | Cancer | PKN3 |
| 468 | Cancer | PLAU |
| 469 | Cancer | PLK1 |
| 470 | Cancer | PML/RARA |
| 471 | Cancer | PMS-1 |
| 472 | Cancer | PMS-2 |
| 473 | Cancer | POLK |
| 474 | Cancer | POU4F2 |
| 475 | Cancer | PPARD |
| 476 | Cancer | PPP2CA |
| 477 | Cancer | PPP2R1A |
| 478 | Cancer | PPP2R1B |
| 479 | Cancer | PRAD-1 |
| 480 | Cancer | PRC |
| 481 | Cancer | PRCA1 |
| 482 | Cancer | Prohibitin |
| 483 | Cancer | Proteasome inhibitors |
| 484 | Cancer | PRKCA |
| 485 | Cancer | PRKRA |
| 486 | Cancer | PRKG1 |
| 487 | Cancer | PSDK1 |
| 488 | Cancer | P-Selectin |
| 489 | Cancer | PTCH |
| 490 | Cancer | PTEN |
| 491 | Cancer | PTGS2 |
| 492 | Cancer | PTK2B |
| 493 | Cancer | PTN (pleiotrophin) |
| 494 | Cancer | RAB6A |
| 495 | Cancer | RAB6B |
| 496 | Cancer | RAB21 |
| 497 | Cancer | RAC1 |
| 498 | Cancer | RAC3 |
| 499 | Cancer | RAF |
| 500 | Cancer | RAF1 |
| 501 | Cancer | RAI |
| 502 | Cancer | RANKL |
| 503 | Cancer | RAR-28 |
| 504 | Cancer | RAS |
| 505 | Cancer | RASL10B (VTS58635) |
| 506 | Cancer | RRAS |
| 507 | Cancer | RAASF1 |
| 508 | Cancer | RB1 |
| 509 | Cancer | RBL2 |
| 510 | Cancer | REL |
| 511 | Cancer | RERG |
| 512 | Cancer | RET |
| 513 | Cancer | REST |
| 514 | Cancer | RFC-1 |
| 515 | Cancer | RHOA |
| 516 | Cancer | RHOB |
| 517 | Cancer | RHOBTB2 |
| 518 | Cancer | RHOM-1 |
| 519 | Cancer | RHOM-2 |
| 520 | Cancer | rhPDGF-BB |
| 521 | Cancer | RNA-R2 |
| 522 | Cancer | ROCK2 |
| 523 | Cancer | ROS1 |
| 524 | Cancer | RTKN |
| 525 | Cancer | RUNX1 |
| 526 | Cancer | RUNX1/CBFA2T1 |
| 527 | Cancer | RUNXIT1 |
| 528 | Cancer | SDCBP |
| 529 | Cancer | SEPT9 |
| 530 | Cancer | Ser/Thr |
| 531 | Cancer | SERPINB5 (MASPIN) |
| 532 | Cancer | SET |
| 533 | Cancer | SHC1 |
| 534 | Cancer | SIRT1 |
| 535 | Cancer | SIS |
| 536 | Cancer | SKI |
| 537 | Cancer | SLUG |
| 538 | Cancer | SMAD1 |
| 539 | Cancer | SMAD2 |
| 540 | Cancer | SMAD3 |
| 541 | Cancer | SMAD4 |
| 542 | Cancer | SMAD7 |
| 543 | Cancer | SMARCA4 |
| 544 | Cancer | SFRP1 |
| 545 | Cancer | SKP2 |
| 546 | Cancer | SNAIL |
| 547 | Cancer | SOCS1 |
| 548 | Cancer | SOS |
| 549 | Cancer | SOX2 |
| 550 | Cancer | SOX9 |
| 551 | Cancer | SPANXC |
| 552 | Cancer | SRC1 |
| 553 | Cancer | v-src |
| 554 | Cancer | SST |
| 555 | Cancer | STAT1 |
| 556 | Cancer | STAT3 |
| 557 | Cancer | STK11 |
| 558 | Cancer | STX2 |
| 559 | Cancer | Survivin |
| 560 | Cancer | SYNE1 |
| 561 | Cancer | TACR1 |
| 562 | Cancer | TAL1 |
| 563 | Cancer | TAL2 |
| 564 | Cancer | TAN1 |
| 565 | Cancer | TCF3/PBX1 |
| 566 | Cancer | TCF8/ZEB1 |
| 567 | Cancer | TET2 |
| 568 | Cancer | TFPI2 |
| 569 | Cancer | TFRC (TfR) |
| 570 | Cancer | TGFB1 |
| 571 | Cancer | TGFB2 |
| 572 | Cancer | TGFBR1 |
| 573 | Cancer | TGFBR2 |
| 574 | Cancer | TGF-α |
| 575 | Cancer | TGFβ |
| 576 | Cancer | TGIF2 |
| 577 | Cancer | TGRC |
| 578 | Cancer | THOC1 |
| 579 | Cancer | THRA1 |
| 580 | Cancer | THRB |
| 581 | Cancer | TIAM1 |
| 582 | Cancer | TIE2 |
| 583 | Cancer | TIF1A |
| 584 | Cancer | TIM3/HAVCR2 |
| 585 | Cancer | TIMP1 |
| 586 | Cancer | TIMP2 |
| 587 | Cancer | TIMP3 |
| 588 | Cancer | TIMP4 |
| 589 | Cancer | TK |
| 590 | Cancer | TLX1 |
| 591 | Cancer | TM1 |
| 592 | Cancer | TMEFF2 |
| 593 | Cancer | TNC |
| 594 | Cancer | TNFAIP3 |
| 595 | Cancer | TNFα |
| 596 | Cancer | TNFRSF1A |
| 597 | Cancer | TNFRSF10A |
| 598 | Cancer | TNFRSF11A (RANK) |
| 599 | Cancer | TOP1 |
| 600 | Cancer | TP73L/p63 |
| 601 | Cancer | TPM1 |
| 602 | Cancer | TRIM2 |
| 603 | Cancer | TRK |
| 604 | Cancer | TRKB |
| 605 | Cancer | TrkC |
| 606 | Cancer | TSC1 |
| 607 | Cancer | TSC2 |
| 608 | Cancer | TSG101 |
| 609 | Cancer | Tubulin beta 3 |
| 610 | Cancer | Tubulin beta 5 |
| 611 | Cancer | TUSC2 |
| 612 | Cancer | Twist |
| 613 | Cancer | TWIST1 |
| 614 | Cancer | Tyr |
| 615 | Cancer | Tyrosine Kinase Enzymes |
| 616 | Cancer | VAV |
| 617 | Cancer | VDR |
| 618 | Cancer | VCAM |
| 619 | Cancer | VEGF |
| 620 | Cancer | VEGFA |
| 621 | Cancer | VHL |
| 622 | Cancer | WAF1 |
| 623 | Cancer | WEE1 |
| 624 | Cancer | WIF1 |
| 625 | Cancer | WNT |
| 626 | Cancer | WNT1 |
| 627 | Cancer | WNT2 |
| 628 | Cancer | WT1 |
| 629 | Cancer | XAF1 |
| 630 | Cancer | XIAP |
| 631 | Cancer | XPA/XPG |
| 632 | Cancer | XPO1 |
| 633 | Cancer | YES1 |
| 634 | Cancer | YWHAE |
| 635 | Cancer | YY1 |
| 636 | Cancer | ZAK (MLT) |
| 637 | Cancer | ZEB2 |
| 638 | Cancer | αv-β3 |
| 639 | Cancer | RAD51 |
| 640 | Cancer | RAD51C |
| 641 | Cancer | PPARβ |
| 642 | Cancer | PPARγ |
| 643 | Cancer | SPHK2 |
| 644 | Cancer | SPHK1 |
| 645 | Cancer | TMFRSF5B |
| 646 | Cancer | STAT6 |
| 647 | Cancer | KLF4 |
| 648 | Cardiovascular/Metabolic Disease | ACC |
| 649 | Cardiovascular/Metabolic Disease | ANGPTL3 |
| 650 | Cardiovascular/Metabolic Disease | Apo(a) |
| 651 | Cardiovascular/Metabolic Disease | APOA1 |
| 652 | Cardiovascular/Metabolic Disease | APOA4 |
| 653 | Cardiovascular/Metabolic Disease | APOA5 |
| 654 | Cardiovascular/Metabolic Disease | ApoB |
| 655 | Cardiovascular/Metabolic Disease | ApoB-100 |
| 656 | Cardiovascular/Metabolic Disease | ApoC I |
| 657 | Cardiovascular/Metabolic Disease | ApoC III |
| 658 | Cardiovascular/Metabolic Disease | APOE |
| 659 | Cardiovascular/Metabolic Disease | BACE1 |
| 660 | Cardiovascular/Metabolic Disease | Citrate lyase |
| 661 | Cardiovascular/Metabolic Disease | DGAT2 |
| 662 | Cardiovascular/Metabolic Disease | endotheal lipase |
| 663 | Cardiovascalar/Metabolic Disease | Factor VII |
| 664 | Cardiovascular/Metabolic Disease | Factor IX/F9 |
| 665 | Cardiovascular/Metabolic Disease | FGFR4 |
| 666 | Cardiovascular/Metabolic Disease | GCGR |
| 667 | Cardiovascular/Metabolic Disease | HDL |
| 668 | Cardiovascular/Metabolic Disease | LDL |
| 669 | Cardiovascular/Metabolic Disease | MTTP |
| 670 | Cardiovascular/Metabolic Disease | PAFAH1B2 |
| 671 | Cardiovascular/Metabolic Disease | PCSK9 |
| 672 | Cardiovascular/Metabolic Disease | PTP-1B |
| 673 | Cardiovascalar/Metabolic Disease | VLDL |
| | Cardiovascular/Metabolic Disease | THP—Thrombopoietin for |
| | | essential thrombocytosis |
| 674 | Eye Disease | ARMS2 |
| 675 | Eye Disease | CFH |
| 676 | Eye Disease | C5 |
| 677 | Eye Disease | ERK1 |
| 678 | Eye Disease | ERK2 |
| 679 | Eye Disease | Il-18 |
| 680 | Eye Disease | NGF (proNGF) |
| 681 | Eye Disease | PDGFC |
| 682 | Eye Disease | RTP801 |
| 683 | Eye Disease | TLR4 |
| 684 | Infectious Disease | ACEE |
| 685 | Infectious Disease | aroA |
| 686 | Infectious Disease | aroC |
| 687 | Infectious Disease | B2M |
| 688 | Infectious Disease | carA |
| 689 | Infectious Disease | CASP1 |
| 690 | Infectious Disease | celB |
| 691 | Infectious Disease | cflA |
| 692 | Infectious Disease | cglA |
| 693 | Infectious Disease | cglE |
| 694 | Infectious Disease | cilA |
| 695 | Infectious Disease | cilB |
| 696 | Infectious Disease | cilC |
| 697 | Infectious Disease | cilD |
| 698 | Infectious Disease | cilE |
| 699 | Infectious Disease | cinA |
| 700 | Infectious Disease | CCL3 |
| 701 | Infectious Disease | CCL4 |
| 702 | Infectious Disease | CCR5 |
| 703 | Infectious Disease | CD14 |
| 704 | Infectious Disease | CD28 |
| 705 | Infectious Disease | CHIT1 |
| 706 | Infectious Disease | coiA |
| 707 | Infectious Disease | comA |
| 708 | Infectious Disease | comC |
| 709 | Infectious Disease | comX |
| 710 | Infectious Disease | CSF3 |
| 711 | Infectious Disease | CTL |
| 712 | Infectious Disease | DDX25 |
| 713 | Infectious Disease | DMC1 |
| 714 | Infectious Disease | Ebola |
| 715 | Infectious Disease | envZ |
| 716 | Infectious Disease | epsA |
| 717 | Infectious Disease | F3 |
| 718 | Infectious Disease | F8 |
| 719 | Infectious Disease | FKBP8 |
| 720 | Infectious Disease | Food borne pathogens |
| 721 | Infectious Disease | H1N1 |
| 722 | Infectious Disease | H3N2 |
| 723 | Infectious Disease | H5N1 |
| 724 | Infectious Disease | HBx |
| 725 | Infectious Disease | Hep-A |
| 726 | Infectious Disease | Hep-B |
| 727 | Infectious Disease | Hep-C |
| 728 | Infectious Disease | HIV |
| 729 | Infectious Disease | HLA-A |
| 730 | Infectious Disease | HLA-B |
| 731 | Infectious Disease | HLA-C |
| 732 | Infectious Disease | HP |
| 733 | Infectious Disease | HSPD1 |
| 734 | Infectious Disease | IDO1 |
| 735 | Infectious Disease | IL1B |
| 736 | Infectious Disease | IL6 |
| 737 | Infectious Disease | IL12RB2 |
| 738 | Infectious Disease | IL15 |
| 739 | Infectious Disease | IL17A |
| 740 | Infectious Disease | IL1RN |
| 741 | Infectious Disease | Influenza RNA-dependent |
| | | RNA polymerase |
| 742 | Infectious Disease | INS |
| 743 | Infectious Disease | LACTB |
| 744 | Infectious Disease | LTA |
| 745 | Infectious Disease | Malaria |
| 746 | Infectious Disease | MBL2 |
| 747 | Infectious Disease | MIF |
| 748 | Infections Disease | miR-122 |
| 749 | Infectious Disease | MMP3 |
| 750 | Infectious Disease | NS1A |
| 751 | Infectious Disease | NS5A |
| 752 | Infectious Disease | ompF |
| 753 | Infectious Disease | ostA |
| 754 | Infectious Disease | pbpG |
| 755 | Infectious Disease | PPIA |
| 756 | Infectious Disease | Protease Inhibitors |
| 757 | Infectious Disease | PRTN3 |
| 758 | Infectious Disease | PTK |
| 759 | Infectious Disease | PTPRC/CD45 |
| 760 | Infectious Disease | pyrC |
| 761 | Infectious Disease | relA |
| 762 | Infectious Disease | retinoic acid receptors/ |
| | | retinoids |
| 763 | Infectious Disease | rpmA |
| 764 | Infectious Disease | rstA |
| 765 | Infectious Disease | RSV |
| 766 | Infectious Disease | RSV |
| 767 | Infectious Disease | SARS |
| 768 | Infectious Disease | secE |
| 769 | Infectious Disease | SELL |
| 770 | Infectious Disease | SERPINA1 |
| 771 | Infectious Disease | SLC11A1 |
| 772 | Infectious Disease | spsC |
| 773 | Infectious Disease | tcdA |
| 774 | Infectious Disease | tcdB |
| 775 | Infectious Disease | TLR2 |
| 776 | Infectious Disease | TLR7 |
| 777 | Infectious Disease | TNF |
| 778 | Infectious Disease | TNFRSF1B |
| 779 | Infectious Disease | TNFRSF8 |
| 780 | Infectious Disease | trmD |
| 781 | Infectious Disease | uppP |
| 782 | Infectious Disease | West Nile |
| 783 | Inflammation | ACEI |
| 784 | Inflammation | ADAMS |
| 785 | Inflammation | ADAMTS |
| 786 | Inflammation | AGER |
| 787 | Inflammation | Aldosterone |
| 788 | Inflammation | ALK5 |
| 789 | Inflammation | Aminoglycoside |
| 790 | Inflammation | ARB |
| 791 | Inflammation | ATG16L1 |
| 792 | Inflammation | BDKRB1 |
| 793 | Inflammation | bFGF |
| 794 | Inflammation | BMP-7 |
| 795 | Inflammation | c-abl |
| 796 | Inflammation | CaMKIV |
| 797 | Inflammation | CASP14 |
| 798 | Inflammation | CCL2/CCL2 receptor |
| 799 | Inflammation | CCL13 |
| 800 | Inflammation | CCN2 |
| 801 | Inflammation | CCR1 |
| 802 | Inflammation | CCR2 |
| 803 | Inflammation | CCR9 |
| 804 | Inflammation | CCR10 |
| 805 | Inflammation | CD97 |
| 806 | Inflammation | COX |
| 807 | Inflammation | CRP |
| 808 | Inflammation | CTGF |
| 809 | Inflammation | CX3CR1 |
| 810 | Inflammation | CXCR-4 |
| 811 | Inflammation | CXCR-7 |
| 812 | Inflammation | Endothelin |
| 813 | Inflammation | ELANE |
| 814 | Inflammation | EPO |
| 815 | Inflammation | F2RL1 |
| 816 | Inflammation | FPR1 |
| 817 | Inflammation | FPR2 |
| 818 | Inflammation | GPR84 |
| 819 | Inflammation | GZMB |
| 820 | Inflammation | Hepcidin (HAMP) |
| 821 | Inflammation | HGF |
| 822 | Inflammation | HRH4 |
| 823 | Inflammation | ICAM-1 |
| 824 | Inflammation | IFNG |
| 825 | Inflammation | IL1 |
| 826 | Inflammation | IL10 |
| 827 | Inflammation | IL12 |
| 828 | Inflammation | IL13 |
| 829 | Inflammation | IL2 |
| 830 | Inflammation | IL4 |
| 831 | Inflammation | Il-5 |
| 832 | Inflammation | IL7 |
| 833 | Inflammation | Integrin α4β7 |
| 834 | Inflammation | JNK |
| 835 | Inflammation | KNG1 |
| 836 | Inflammation | MAPK14 |
| 837 | Inflammation | MCP1 |
| 838 | Inflammation | M-CSF |
| 839 | Inflammation | MIF1 |
| 840 | Inflammation | MYD88 |
| 841 | Inflammation | Nitric Oxide |
| 842 | Inflammation | NOD2 |
| 843 | Inflammation | NR1H2 |
| 844 | Inflammation | P38 MAPK |
| 845 | Inflammation | PAI-1 |
| 846 | Inflammation | PLA2G2D |
| 847 | Inflammation | PLA2G7 |
| 848 | Inflammation | PLA2G10 |
| 849 | Inflammation | plasminogen |
| 850 | Inflammation | PLCγ |
| 851 | Inflammation | PPIG |
| 852 | Inflammation | PPARα |
| 853 | Inflammation | PSGL-1 |
| 854 | Inflammation | PTGDR |
| 855 | Inflammation | PTGDR2 |
| 856 | Inflammation | Rantes (CCL5) |
| 857 | Inflammation | Renin |
| 858 | Inflammation | ROCK (Rho-kinase) |
| 859 | Inflammation | SAA1 |
| 860 | Inflammation | SAP |
| 861 | Inflammation | SCGB1A1 |
| 862 | Inflammation | SELPLG |
| 863 | Inflammation | Smads (1, 2, 3, 5) |
| 864 | Inflammation | SYK |
| 865 | Inflammation | TLR9 |
| 866 | Inflammation | TSLP |
| 867 | Inflammation | TNFAIP6 |
| 868 | Inflammation | TNFAIP8L2 |
| 869 | Inflammation | tpa |
| 870 | Inflammation | uPA |
| 871 | Inflammation | Vasopeptidase |
| 872 | Inflammation | VLA-4 |
| 873 | Inflammation | XBP1 |
| 874 | Neurological Disease | alpha-synuclein |
| 875 | Neurological Disease | ApoE 4 |
| 876 | Neurological Disease | APP |
| 877 | Neurological Disease | Beta amyloid |
| 878 | Neurological Disease | CDK5R2 |
| 879 | Neurological Disease | CLU |
| 880 | Neurological Disease | COX2 |
| 881 | Neurological Disease | CR1 |
| 882 | Neurological Disease | ErbB |
| 883 | Neurological Disease | FRA10AC1 |
| 884 | Neurological Disease | GBA |
| 885 | Neurological Disease | GNAS |
| 886 | Neurological Disease | GPCR |
| 887 | Neurological Disease | GRM1 |
| 888 | Neurological Disease | GUSB |
| 889 | Neurological Disease | has-mir-29b |
| 890 | Neurological Disease | has-mir-29c |
| 891 | Neurological Disease | HDAC3 |
| 892 | Neurological Disease | hnRNPA1 |
| 893 | Neurological Disease | hnRNPA2B1 |
| 894 | Neurological Disease | hsa-miR-137 |
| 895 | Neurological Disease | HTT |
| 896 | Neurological Disease | IAPP |
| 897 | Neurological Disease | LRRK2 |
| 898 | Neurological Disease | MAPT |
| 899 | Neurological Disease | MBP |
| 900 | Neurological Disease | MDK (Midkine) |
| 901 | Neurological Disease | MT-ATP6 |
| 902 | Neurological Disease | PARK |
| 903 | Neurological Disease | PARK7 |
| 904 | Neurological Disease | PBP |
| 905 | Neurological Disease | PDE1B |
| 906 | Neurological Disease | PICALM |
| 907 | Neurological Disease | PINK1 |
| 908 | Neurological Disease | PON1 |
| 909 | Neurological Disease | PPARGC1B |
| 910 | Neurological Disease | PRNP |
| 911 | Neurological Disease | PSEN1 |
| 912 | Neurological Disease | PSEN2 |
| 913 | Neurological Disease | RAGE |
| 914 | Neurological Disease | SERPINA3 |
| 915 | Neurological Disease | SNCA |
| 916 | Neurological Disease | SOD1 |
| 917 | Neurological Disease | SPON1 |
| 918 | Neurological Disease | SPP1 |
| 919 | Neurological Disease | STH |
| 920 | Neurological Disease | Supt4h |
| 921 | Neurological Disease | Tau |
| 922 | Neurological Disease | TOMM40 |
| 923 | Neurological Disease | TUBA3 |
| 924 | Neurological Disease | Ubiquilin-2 |
| 925 | Rare Disease | AAT |
| 926 | Rare Disease | ABCG5 |
| 927 | Rare Disease | ACHE |
| 928 | Rare Disease | ADA |
| 929 | Rare Disease | AGXT |
| 930 | Rare Disease | AIRE |
| 931 | Rare Disease | ALAS-1 |
| 932 | Rare Disease | ALDH2 |
| 933 | Rare Disease | alpha-1 antritrypsisn |
| 934 | Rare Disease | AMPH |
| 935 | Rare Disease | antithrombin |
| 936 | Rare Disease | AQP2 |
| 937 | Rare Disease | ASPA |
| 938 | Rare Disease | APT7A |
| 939 | Rare Disease | ATP7B |
| 940 | Rare Disease | AVPR2 |
| 941 | Rare Disease | BSCL2 |
| 942 | Rare Disease | C1S |
| 943 | Rare Disease | CCL3L1 |
| 944 | Rare Disease | CD79A |
| 945 | Rare Disease | CTLA4 |
| 946 | Rare Disease | CYB5R3 |
| 947 | Rare Disease | CYP117A1 |
| 948 | Rare Disease | CYBB |
| 949 | Rare Disease | CYP21A2 |
| 950 | Rare Disease | CYP27A1 |
| 951 | Rare Disease | DMPK |
| 952 | Rare Disease | ENO2 |
| 953 | Rare Disease | F2 |
| 954 | Rare Disease | F5 |
| 955 | Rare Disease | F10 |
| 956 | Rare Disease | FGF23 |
| 957 | Rare Disease | FRAXA |
| 958 | Rare Disease | FRAXE |
| 959 | Rare Disease | GAA |
| 960 | Rare Disease | GAD1 |
| 961 | Rare Disease | GCCR |
| 962 | Rare Disease | GCK |
| 963 | Rare Disease | GDNF |
| 964 | Rare Disease | GFAP |
| 965 | Rare Disease | GH1 |
| 966 | Rare Disease | GHR |
| 967 | Rare Disease | GJB1 |
| 968 | Rare Disease | GLA |
| 969 | Rare Disease | GLRA1 |
| 970 | Rare Disease | GYS2 |
| 971 | Rare Disease | HADHA |
| 972 | Rare Disease | HFE |
| 973 | Rare Disease | IGES |
| 974 | Rare Disease | IPW |
| 975 | Rare Disease | KCNJ2 |
| 976 | Rare Disease | KRT6A (Keratin K6a) |
| 977 | Rare Disease | KRT81 |
| 978 | Rare Disease | KRT86 |
| 979 | Rare Disease | LMAN1 |
| 980 | Rare Disease | LMNA |
| 981 | Rare Disease | MPL |
| 982 | Rare Disease | MPZ |
| 983 | Rare Disease | NEU1 |
| 984 | Rare Disease | NPC1 |
| 985 | Rare Disease | NPC2 |
| 986 | Rare Disease | NR0B1 |
| 987 | Rare Disease | NR3C2 |
| 988 | Rare Disease | PKK |
| 989 | Rare Disease | PMP22 |
| 990 | Rare Disease | PYGM |
| 991 | Rare Disease | RETN |
| 992 | Rare Disease | SAG |
| 993 | Rare Disease | SCNN1A |
| 994 | Rare Disease | SH2D1A |
| 995 | Rare Disease | SLC2A1 (Glut1) |
| 996 | Rare Disease | SMN2 |
| 997 | Rare Disease | SMPD1 |
| 998 | Rare Disease | SNRPN |
| 999 | Rare Disease | THBD |
| 1000 | Rare Disease | STAR |
| 1001 | Rare Disease | SYP |
| 1002 | Rare Disease | TRD |
| 1003 | Rare Disease | TSHB |
| 1004 | Rare Disease | Tmprss6 |
| 1005 | Rare Disease | TTR |
| 1006 | Rare Disease | UBE3A |
| 1007 | Rare Disease | WAS |
| 1008 | Rare Disease | WRN |
| 1009 | Rare Disease | Dentatorubropallidoluysian |
| | | Atrophy |
| 1010 | Rare Disease | Huntington′s Disease |
| 1011 | Rare Disease | Spinobulbar Muscular |
| | | Atrophy |
| 1012 | Rare Disease | SCA1 (Spinocerebellar Ataxia |
| | | Type 1) |
| 1013 | Rare Disease | SCA2 (Spinocerebellar Ataxia |
| | | Type 2) |
| 1014 | Rare Disease | SCA3 (Spinocerebellar Ataxia |
| | | Type 3 or Machado-Joseph |
| | | Disease) |
| 1015 | Rare Disease | SCA6 (Spinocerebellar Ataxia |
| | | Type 6) |
| 1016 | Rare Disease | SCA7 (Spinocerebellar Ataxia |
| | | Type 7) |
| 1017 | Rare Disease | Fragile X Syndrome |
| 1018 | Rare Disease | Fragile XE Mental |
| | | Retardation |
| 1019 | Rare Disease | Friedreich′s Ataxia |
| 1020 | Rare Disease | Myotonic Dystrophy |
| 1021 | Rare Disease | Spinocerebellar Ataxia |
| | | Type 8 |
| 1022 | Rare Disease | Spinocerebellar Ataxia |
| | | Type 12 |
| 1023 | Rare Disease | SPT4 |
| 1024 | Rare Disease | ATN1 |
| 1025 | Rare Disease | DRPLA |
| 1026 | Rare Disease | HTT |
| 1027 | Rare Disease | ATXN1 |
| 1028 | Rare Disease | ATXN2 |
| 1029 | Rare Disease | ATXN3 |
| 1030 | Rare Disease | CACNA1A |
| 1031 | Rare Disease | ATXN7 |
| 1032 | Rare Disease | TBP |
| 1033 | Rare Disease | FMR1 |
| 1034 | Rare Disease | AFF2 |
| 1035 | Rare Disease | FXN |
| 1036 | Rare Disease | SCA8 |
| 1037 | Rare Disease | PPP2R2B |
| 1038 | Stem Cells | Cancer Stem Cells |
| 1039 | Stem Cells | Cardiac Stem Cells |
| 1040 | Stem Cells | Kidney Stem Cells |
| 1041 | Stem Cells | Embryonic Stem Cells |
| 1042 | Stem Cells | Tissue Stem Cells |
| 1043 | Stem Cells | Induced Pluripotent Stem |
| | | Cells |
| 1044 | Stem Cells | Blood Stem Cells |
| 1045 | Stem Cells | Mescenchymal Stem Cells |
| 1046 | Stem Cells | Cord Blood Stem Cells |

Non-Cancer Targets

The present invention is not limited to the targeting of cancer genes. The methods and compositions of the present invention find use in the targeting of any gene that it is desirable to down regulate the expression of. For example, targets for immune and/or surface antigens or immune surveillance targets, angiogenic receptors, proteins and factors (kinases, heat shock, hypoxic, oxidative stress gene/protein targets), monogenic diseases, inflammation, gene transcription (transcription factors, cis regulatory elements), cell recognition receptors, cell signaling receptors, cell death (autophagy, necrosis, apoptosis), cell adhesion, survival targets (resistance), metastases targets (brain, primary to secondary tumors), chemokines/cytokines, EMT/MET, immune cell activation factors, multidrug resistance, viral proteins and viral recognition proteins, psoriasis, dermatitis and eczema

Extracellular matrix, stromal or connective tissue genes/proteins, coagulation factors and platelet aggregation or platelet overproduction, and growth factors.

For example, in some embodiments, the genes to be targeted include, but are not limited to, an immunoglobulin or antibody gene, a clotting factor gene, a protease, a pituitary hormone, a protease inhibitor, a growth factor, a somatomedian, a gonadotrophin, a chemotactin, a chemokine, a plasma protein, a plasma protease inhibitor, an interleukin, an interferon, a cytokine, a transcription factor, or a pathogen target (e.g., a viral gene, a bacterial gene, a microbial gene, a fungal gene).

In other embodiments and gene from a pathogen is targeted. Exemplary pathogens include, but are not limited to, Human Immunodeficiency virus (CD4, APOBEC3G, Vif, LEDGF/p75), Hepatitis B virus, hepatitis C virus (SR-B1, scavenger receptor type B1; CLDN-1, claudin-1; OCLN, occluding), hepatitis A virus, respiratory syncytial virus, pathogens involved in severe acute respiratory syndrome, west nile virus, and food borne pathogens (e.g., E. coli).

The lists of Cancer and Non-Cancer targets from above is intended to be specific and accurate, but in addition to the targets above we have further found and we describe in even greater detail the targets listed below, comprising both cancer and non-cancer targets, presented in no particular order. These targets are especially well suited for DNAi targeting and therapy. The preferred list of targets is provided with the sections that follow which provided detailed descriptions of over 40 genes. These gene targets are numbered below, 1-30. Included with a description of many of these preferred targets are the background relevance of the gene, gene identification, the targeted oligonucleotide sequences, the hot zones, and the 5′ upstream genetic code.

### EXPERIMENTALS

These examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); and ° C. (degrees Centigrade).

1) Survivin. Survivin (BIRC5) also called buloviral inhibitor of apoptosis repeat-containing 5 is a member of the inhibitor of apoptosis family that is expressed during mitosis in a cell cycle-dependent manner. Survivin is localized to different components of the mitotic apparatus, plays an important role in both cell division and inhibition of apoptosis. Survivin is not expressed in normal adult tissue, but is widely expressed in a majority of cancers (Fukuda and Pelus, Mol Cancer Ther 2006; 5 1087-1098), often with poor prognosis. Survivin inhibits caspase activation, the key effector enzyme in programmed cell death, and as a result there is uncontrolled growth and drug resistance. The inhibition of survivin leads to increased apoptosis and decreased tumor growth and sensitizes cells to various therapeutic interventions including chemotherapies and targeted therapies against cancer targets. Survivin expression is increased in tumors and regulated by the cell cycle (expressed in mitosis in a cell cycle dependent manner); expression is also linked to p53 and is targeted by the WNT1 pathway and is upregulated by β-catenin. A review of approaches targeted against survivin may be found in “Targeting surviving in cancer: a patent review” (Expert Opinion on Therapeutic Patents, December 2010, Vol. 20, No. 12: Pages 1723-1737).

An antisense therapeutic being developed (LY2181308) downregulates survivin expression in human cancer cells derived from lung, colon, pancreas, liver, breast, prostate, ovary, cervix, skin, and brain as measured by quantitative RT-PCR and immunoblotting analysis (Carrasco et al., Mol Cancer Ther 2011; 10(2); 221-32). Specific inhibition of survivin expression in multiple cancer cell lines induced caspase-3-dependent apoptosis, cell cycle arrest in the G2-M phase, and multinucleated cells and sensitized tumor cells to chemotherapeutic-induced apoptosis. In an in vivo human xenograft tumor model, LY2181308 produced significant antitumor activity as compared with saline or its sequence-specific control oligonucleotide and sensitized to gemcitabine, paclitaxel, and docetaxel with inhibition of surviving expression in xenograft tumors. LY2181308 is being evaluated in a clinical setting (Phase II) in combination with docetaxel for the treatment of prostate cancer.

Protein: Survivin Gene: BIRC5 (Homo sapiens, chromosome 17, 76210277-76221716 [NCBI Reference Sequence: NC_{—}000017.10]; start site location: 76210398; strand: positive)

Gene Identification

| | |
|---|---|
| GeneID | 332 |
| HGNC | 593 |
| HPRD | 04520 |
| MIM | 603352 |

Targeted Sequences

| | | | Relative |
|---|---|---|---|
| | | | upstream |
| | | | location |
| Se- | | | to gene |
| quence | Design | | start |
| ID No: | ID | Sequence (5′-3′) | site |
| 1 | SU1 | GAGCGCACGCCCTCTTAGGCGG | 73 |
| 75 | SU2 | CACCCCGAGGTACGATCAGTGCGTACC | 2990 |
| 105 | SU3 | GACATCGCTGTCCCGGCGAGTACATCGTT | 665 |
| 155 | SU1_02 | GAGCGCACGCCCTCTTAGGCG | 73 |
| 229 | SU1_03 | GAGCGCACGCCCTCTTAGGCGGTCCA | 73 |
| 303 | | GTCGCCCCTGGGTCCTGCTGATTGGC | 1918 |
| 322 | | CAGCGAGCCTGGGCCCCATCGGCACATCT | 2905 |
| 357 | | CCCGCGGCCTTCTGGGAGTAGAGGC | 102 |
| 431 | | TCCCGGCGAGTACATCGTTGACTGCACG | 675 |
| 481 | | AACCTCCTCCCCGCCACGGGTT | 1229 |

Target Shift Sequences

| | | Relative |
|---|---|---|
| | | upstream |
| | | location |
| Sequence | | to gene |
| ID No: | Sequence (5′-3′) | start site |
| 1 | GAGCGCACGCCCTCTTAGGCGG | 77 |
| 2 | AGCGCACGCCCTCTTAGGCG | 78 |
| 3 | GCGCACGCCCTCTTAGGCGG | 79 |
| 4 | CGCACGCCCTCTTAGGCGGT | 80 |
| 5 | GCACGCCCTCTTAGGCGGTC | 81 |
| 6 | CACGCCCTCTTAGGCGGTCC | 82 |
| 7 | ACGCCCTCTTAGGCGGTCCA | 83 |
| 8 | CGCCCTCTTAGGCGGTCCAC | 84 |
| 9 | GCCCTCTTAGGCGGTCCACC | 85 |
| 10 | CCCTCTTAGGCGGTCCACCC | 86 |
| 11 | CCTCTTAGGCGGTCCACCCC | 87 |
| 12 | CTCTTAGGCGGTCCACCCCC | 88 |
| 13 | TCTTAGGCGGTCCACCCCCC | 89 |
| 14 | CTTAGGCGGTCCACCCCCCG | 90 |
| 15 | TTAGGCGGTCCACCCCCCGC | 91 |
| 16 | TAGGCGGTCCACCCCCCGCG | 92 |
| 17 | AGGCGGTCCACCCCCCGCGG | 93 |
| 18 | GGCGGTCCACCCCCCGCGGC | 94 |
| 19 | GCGGTCCACCCCCCGCGGCC | 95 |
| 20 | CGGTCCACCCCCCGCGGCCT | 96 |
| 21 | GGTCCACCCCCCGCGGCCTT | 97 |
| 22 | GTCCACCCCCCGCGGCCTTC | 98 |
| 23 | TCCACCCCCCGCGGCCTTCT | 99 |
| 24 | CCACCCCCCGCGGCCTTCTG | 100 |
| 25 | CACCCCCCGCGGCCTTCTGG | 101 |
| 26 | ACCCCCCGCGGCCTTCTGGG | 102 |
| 27 | CCCCCCGCGGCCTTCTGGGA | 103 |
| 28 | CCCCCGCGGCCTTCTGGGAG | 104 |
| 29 | CCCCGCGGCCTTCTGGGAGT | 105 |
| 30 | CCCGCGGCCTTCTGGGAGTA | 106 |
| 31 | CCGCGGCCTTCTGGGAGTAG | 107 |
| 32 | CGCGGCCTTCTGGGAGTAGA | 108 |
| 33 | GCGGCCTTCTGGGAGTAGAG | 109 |
| 34 | CGGCCTTCTGGGAGTAGAGG | 110 |
| 35 | GGAGCGCACGCCCTCTTAGG | 76 |
| 36 | GGGAGCGCACGCCCTCTTAG | 75 |
| 37 | CGGGAGCGCACGCCCTCTTA | 74 |
| 38 | TCGGGAGCGCACGCCCTCTT | 73 |
| 39 | GTCGGGAGCGCACGCCCTCT | 72 |
| 40 | TGTCGGGAGCGCACGCCCTC | 71 |
| 41 | ATGTCGGGAGCGCACGCCCT | 70 |
| 42 | CATGTCGGGAGCGCACGCCC | 69 |
| 43 | GCATGTCGGGAGCGCACGCC | 68 |
| 44 | GGCATGTCGGGAGCGCACGC | 67 |
| 45 | GGGCATGTCGGGAGCGCACG | 66 |
| 46 | GGGGCATGTCGGGAGCGCAC | 65 |
| 47 | CGGGGCATGTCGGGAGCGCA | 64 |
| 48 | GCGGGGCATGTCGGGAGCGC | 63 |
| 49 | CGCGGGGCATGTCGGGAGCG | 62 |
| 50 | CCGCGGGGCATGTCGGGAGC | 61 |
| 51 | GCCGCGGGGCATGTCGGGAG | 60 |
| 52 | CGCCGCGGGGCATGTCGGGA | 59 |
| 53 | GCGCCGCGGGGCATGTCGGG | 58 |
| 54 | CGCGCCGCGGGGCATGTCGG | 57 |
| 55 | GCGCGCCGCGGGGCATGTCG | 56 |
| 56 | GGCGCGCCGCGGGGCATGTC | 55 |
| 57 | TGGCGCGCCGCGGGGCATGT | 54 |
| 58 | ATGGCGCGCCGCGGGGCATG | 53 |
| 59 | AATGGCGCGCCGCGGGGCAT | 52 |
| 60 | TAATGGCGCGCCGCGGGGCA | 51 |
| 61 | TTAATGGCGCGCCGCGGGGC | 50 |
| 62 | GTTAATGGCGCGCCGCGGGG | 49 |
| 63 | GGTTAATGGCGCGCCGCGGG | 48 |
| 64 | CGGTTAATGGCGCGCCGCGG | 47 |
| 65 | GCGGTTAATGGCGCGCCGCG | 46 |
| 66 | GGCGGTTAATGGCGCGCCGC | 45 |
| 67 | TGGCGGTTAATGGCGCGCCG | 44 |
| 68 | CTGGCGGTTAATGGCGCGCC | 43 |
| 69 | TCTGGCGGTTAATGGCGCGC | 42 |
| 70 | ATCTGGCGGTTAATGGCGCG | 41 |
| 71 | AATCTGGCGGTTAATGGCGC | 40 |
| 72 | AAATCTGGCGGTTAATGGCG | 39 |
| 73 | CAAATCTGGCGGTTAATGGC | 38 |
| 74 | TCAAATCTGGCGGTTAATGG | 37 |
| 75 | CACCCCGAGGTACGATCAGTGCGTACC | 2994 |
| 76 | ACCCCGAGGTACGATCAGTG | 2995 |
| 77 | CCCCGAGGTACGATCAGTGC | 2996 |
| 78 | CCCGAGGTACGATCAGTGCG | 2997 |
| 79 | CCGAGGTACGATCAGTGCGT | 2998 |
| 80 | CGAGGTACGATCAGTGCGTA | 2999 |
| 81 | GAGGTACGATCAGTGCGTAC | 3000 |
| 82 | AGGTACGATCAGTGCGTACC | 3001 |
| 83 | GGTACGATCAGTGCGTACCA | 3002 |
| 84 | GTACGATCAGTGCGTACCAA | 3003 |
| 85 | TACGATCAGTGCGTACCAAG | 3004 |
| 86 | ACGATCAGTGCGTACCAAGT | 3005 |
| 87 | CGATCAGTGCGTACCAAGTA | 3006 |
| 88 | GATCAGTGCGTACCAAGTAC | 3007 |
| 89 | ATCAGTGCGTACCAAGTACA | 3008 |
| 90 | TCAGTGCGTACCAAGTACAT | 3009 |
| 91 | CAGTGCGTACCAAGTACATA | 3010 |
| 92 | CCACCCCGAGGTACGATCAG | 2993 |
| 93 | CCCACCCCGAGGTACGATCA | 2992 |
| 94 | TCCCACCCCGAGGTACGATC | 2991 |
| 95 | CTCCCACCCCGAGGTACGAT | 2990 |
| 96 | TCTCCCACCCCGAGGTACGA | 2989 |
| 97 | TTCTCCCACCCCGAGGTACG | 2988 |
| 98 | CTTCTCCCACCCCGAGGTAC | 2987 |
| 99 | TCTTCTCCCACCCCGAGGTA | 2986 |
| 100 | CTCTTCTCCCACCCCGAGGT | 2985 |
| 101 | TCTCTTCTCCCACCCCGAGG | 2984 |
| 102 | CTCTCTTCTCCCACCCCGAG | 2983 |
| 103 | CCTCTCTTCTCCCACCCCGA | 2982 |
| 104 | CCCTCTCTTCTCCCACCCCG | 2981 |
| 105 | GACATCGCTGTCCCGGCGAGTACATCGTT | 669 |
| 106 | ACATCGCTGTCCCGGCGAGT | 670 |
| 107 | CATCGCTGTCCCGGCGAGTA | 671 |
| 108 | ATCGCTGTCCCGGCGAGTAC | 672 |
| 109 | TCGCTGTCCCGGCGAGTACA | 673 |
| 110 | CGCTGTCCCGGCGAGTACAT | 674 |
| 111 | GCTGTCCCGGCGAGTACATC | 675 |
| 112 | CTGTCCCGGCGAGTACATCG | 676 |
| 113 | TGTCCCGGCGAGTACATCGT | 677 |
| 114 | GTCCCGGCGAGTACATCGTT | 678 |
| 115 | TCCCGGCGAGTACATCGTTG | 679 |
| 116 | CCCGGCGAGTACATCGTTGA | 680 |
| 117 | CCGGCGAGTACATCGTTGAC | 681 |
| 118 | CGGCGAGTACATCGTTGACT | 682 |
| 119 | GGCGAGTACATCGTTGACTG | 683 |
| 120 | GCGAGTACATCGTTGACTGC | 684 |
| 121 | CGAGTACATCGTTGACTGCA | 685 |
| 122 | GAGTACATCGTTGACTGCAC | 686 |
| 123 | AGTACATCGTTGACTGCACG | 687 |
| 124 | GTACATCGTTGACTGCACGA | 688 |
| 125 | TACATCGTTGACTGCACGAC | 689 |
| 126 | ACATCGTTGACTGCACGACC | 690 |
| 127 | CATCGTTGACTGCACGACCT | 691 |
| 128 | ATCGTTGACTGCACGACCTG | 692 |
| 129 | TCGTTGACTGCACGACCTGG | 693 |
| 130 | CGTTGACTGCACGACCTGGG | 694 |
| 131 | GTTGACTGCACGACCTGGGT | 695 |
| 132 | TTGACTGCACGACCTGGGTT | 696 |
| 133 | TGACTGCACGACCTGGGTTT | 697 |
| 134 | GACTGCACGACCTGGGTTTC | 698 |
| 135 | ACTGCACGACCTGGGTTTCC | 699 |
| 136 | CTGCACGACCTGGGTTTCCA | 700 |
| 137 | TGCACGACCTGGGTTTCCAG | 701 |
| 138 | GCACGACCTGGGTTTCCAGG | 702 |
| 139 | CACGACCTGGGTTTCCAGGA | 703 |
| 140 | ACGACCTGGGTTTCCAGGAG | 704 |
| 141 | CGACCTGGGTTTCCAGGAGG | 705 |
| 142 | AGACATCGCTGTCCCGGCGA | 668 |
| 143 | CAGACATCGCTGTCCCGGCG | 667 |
| 144 | GCAGACATCGCTGTCCCGGC | 666 |
| 145 | AGCAGACATCGCTGTCCCGG | 665 |
| 146 | CAGCAGACATCGCTGTCCCG | 664 |
| 147 | GCAGCAGACATCGCTGTCCC | 663 |
| 148 | TGCAGCAGACATCGCTGTCC | 662 |
| 149 | GTGCAGCAGACATCGCTGTC | 661 |
| 150 | AGTGCAGCAGACATCGCTGT | 660 |
| 151 | GAGTGCAGCAGACATCGCTG | 659 |
| 152 | GGAGTGCAGCAGACATCGCT | 658 |
| 153 | TGGAGTGCAGCAGACATCGC | 657 |
| 154 | ATGGAGTGCAGCAGACATCG | 656 |
| 155 | GAGCGCACGCCCTCTTAGGCG | 77 |
| 156 | AGCGCACGCCCTCTTAGGCG | 78 |
| 157 | GCGCACGCCCTCTTAGGCGG | 79 |
| 158 | CGCACGCCCTCTTAGGCGGT | 80 |
| 159 | GCACGCCCTCTTAGGCGGTC | 81 |
| 160 | CACGCCCTCTTAGGCGGTCC | 82 |
| 161 | ACGCCCTCTTAGGCGGTCCA | 83 |
| 162 | CGCCCTCTTAGGCGGTCCAC | 84 |
| 163 | GCCCTCTTAGGCGGTCCACC | 85 |
| 164 | CCCTCTTAGGCGGTCCACCC | 86 |
| 165 | CCTCTTAGGCGGTCCACCCC | 87 |
| 166 | CTCTTAGGCGGTCCACCCCC | 88 |
| 167 | TCTTAGGCGGTCCACCCCCC | 89 |
| 168 | CTTAGGCGGTCCACCCCCCG | 90 |
| 169 | TTAGGCGGTCCACCCCCCGC | 91 |
| 170 | TAGGCGGTCCACCCCCCGCG | 92 |
| 171 | AGGCGGTCCACCCCCCGCGG | 93 |
| 172 | GGCGGTCCACCCCCCGCGGC | 94 |
| 173 | GCGGTCCACCCCCCGCGGCC | 95 |
| 174 | CGGTCCACCCCCCGCGGCCT | 96 |
| 175 | GGTCCACCCCCCGCGGCCTT | 97 |
| 176 | GTCCACCCCCCGCGGCCTTC | 98 |
| 177 | TCCACCCCCCGCGGCCTTCT | 99 |
| 178 | CCACCCCCCGCGGCCTTCTG | 100 |
| 179 | CACCCCCCGCGGCCTTCTGG | 101 |
| 180 | ACCCCCCGCGGCCTTCTGGG | 102 |
| 181 | CCCCCCGCGGCCTTCTGGGA | 103 |
| 182 | CCCCCGCGGCCTTCTGGGAG | 104 |
| 183 | CCCCGCGGCCTTCTGGGAGT | 105 |
| 184 | CCCGCGGCCTTCTGGGAGTA | 106 |
| 185 | CCGCGGCCTTCTGGGAGTAG | 107 |
| 186 | CGCGGCCTTCTGGGAGTAGA | 108 |
| 187 | GCGGCCTTCTGGGAGTAGAG | 109 |
| 188 | CGGCCTTCTGGGAGTAGAGG | 110 |
| 189 | GGAGCGCACGCCCTCTTAGG | 76 |
| 190 | GGGAGCGCACGCCCTCTTAG | 75 |
| 191 | CGGGAGCGCACGCCCTCTTA | 74 |
| 192 | TCGGGAGCGCACGCCCTCTT | 73 |
| 193 | GTCGGGAGCGCACGCCCTCT | 72 |
| 194 | TGTCGGGAGCGCACGCCCTC | 71 |
| 195 | ATGTCGGGAGCGCACGCCCT | 70 |
| 196 | CATGTCGGGAGCGCACGCCC | 69 |
| 197 | GCATGTCGGGAGCGCACGCC | 68 |
| 198 | GGCATGTCGGGAGCGCACGC | 67 |
| 199 | GGGCATGTCGGGAGCGCACG | 66 |
| 200 | GGGGCATGTCGGGAGCGCAC | 65 |
| 201 | CGGGGCATGTCGGGAGCGCA | 64 |
| 202 | GCGGGGCATGTCGGGAGCGC | 63 |
| 203 | CGCGGGGCATGTCGGGAGCG | 62 |
| 204 | CCGCGGGGCATGTCGGGAGC | 61 |
| 205 | GCCGCGGGGCATGTCGGGAG | 60 |
| 206 | CGCCGCGGGGCATGTCGGGA | 59 |
| 207 | GCGCCGCGGGGCATGTCGGG | 58 |
| 208 | CGCGCCGCGGGGCATGTCGG | 57 |
| 209 | GCGCGCCGCGGGGCATGTCG | 56 |
| 210 | GGCGCGCCGCGGGGCATGTC | 55 |
| 211 | TGGCGCGCCGCGGGGCATGT | 54 |
| 212 | ATGGCGCGCCGCGGGGCATG | 53 |
| 213 | AATGGCGCGCCGCGGGGCAT | 52 |
| 214 | TAATGGCGCGCCGCGGGGCA | 51 |
| 215 | TTAATGGCGCGCCGCGGGGC | 50 |
| 216 | GTTAATGGCGCGCCGCGGGG | 49 |
| 217 | GGTTAATGGCGCGCCGCGGG | 48 |
| 218 | CGGTTAATGGCGCGCCGCGG | 47 |
| 219 | GCGGTTAATGGCGCGCCGCG | 46 |
| 220 | GGCGGTTAATGGCGCGCCGC | 45 |
| 221 | TGGCGGTTAATGGCGCGCCG | 44 |
| 222 | CTGGCGGTTAATGGCGCGCC | 43 |
| 223 | TCTGGCGGTTAATGGCGCGC | 42 |
| 224 | ATCTGGCGGTTAATGGCGCG | 41 |
| 225 | AATCTGGCGGTTAATGGCGC | 40 |
| 226 | AAATCTGGCGGTTAATGGCG | 39 |
| 227 | CAAATCTGGCGGTTAATGGC | 38 |
| 228 | TCAAATCTGGCGGTTAATGG | 37 |
| 229 | GAGCGCACGCCCTCTTAGGCGGTCCA | 77 |
| 230 | AGCGCACGCCCTCTTAGGCG | 78 |
| 231 | GCGCACGCCCTCTTAGGCGG | 79 |
| 232 | CGCACGCCCTCTTAGGCGGT | 80 |
| 233 | GCACGCCCTCTTAGGCGGTC | 81 |
| 234 | CACGCCCTCTTAGGCGGTCC | 82 |
| 235 | ACGCCCTCTTAGGCGGTCCA | 83 |
| 236 | CGCCCTCTTAGGCGGTCCAC | 84 |
| 237 | GCCCTCTTAGGCGGTCCACC | 85 |
| 238 | CCCTCTTAGGCGGTCCACCC | 86 |
| 239 | CCTCTTAGGCGGTCCACCCC | 87 |
| 240 | CTCTTAGGCGGTCCACCCCC | 88 |
| 241 | TCTTAGGCGGTCCACCCCCC | 89 |
| 242 | CTTAGGCGGTCCACCCCCCG | 90 |
| 243 | TTAGGCGGTCCACCCCCCGC | 91 |
| 244 | TAGGCGGTCCACCCCCCGCG | 92 |
| 245 | AGGCGGTCCACCCCCCGCGG | 93 |
| 246 | GGCGGTCCACCCCCCGCGGC | 94 |
| 247 | GCGGTCCACCCCCCGCGGCC | 95 |
| 248 | CGGTCCACCCCCCGCGGCCT | 96 |
| 249 | GGTCCACCCCCCGCGGCCTT | 97 |
| 250 | GTCCACCCCCCGCGGCCTTC | 98 |
| 251 | TCCACCCCCCGCGGCCTTCT | 99 |
| 252 | CCACCCCCCGCGGCCTTCTG | 100 |
| 253 | CACCCCCCGCGGCCTTCTGG | 101 |
| 254 | ACCCCCCGCGGCCTTCTGGG | 102 |
| 255 | CCCCCCGCGGCCTTCTGGGA | 103 |
| 256 | CCCCCGCGGCCTTCTGGGAG | 104 |
| 257 | CCCCGCGGCCTTCTGGGAGT | 105 |
| 258 | CCCGCGGCCTTCTGGGAGTA | 106 |
| 259 | CCGCGGCCTTCTGGGAGTAG | 107 |
| 260 | CGCGGCCTTCTGGGAGTAGA | 108 |
| 261 | GCGGCCTTCTGGGAGTAGAG | 109 |
| 262 | CGGCCTTCTGGGAGTAGAGG | 110 |
| 263 | GGAGCGCACGCCCTCTTAGG | 76 |
| 264 | GGGAGCGCACGCCCTCTTAG | 75 |
| 265 | CGGGAGCGCACGCCCTCTTA | 74 |
| 266 | TCGGGAGCGCACGCCCTCTT | 73 |
| 267 | GTCGGGAGCGCACGCCCTCT | 72 |
| 268 | TGTCGGGAGCGCACGCCCTC | 71 |
| 269 | ATGTCGGGAGCGCACGCCCT | 70 |
| 270 | CATGTCGGGAGCGCACGCCC | 69 |
| 271 | GCATGTCGGGAGCGCACGCC | 68 |
| 272 | GGCATGTCGGGAGCGCACGC | 67 |
| 273 | GGGCATGTCGGGAGCGCACG | 66 |
| 274 | GGGGCATGTCGGGAGCGCAC | 65 |
| 275 | CGGGGCATGTCGGGAGCGCA | 64 |
| 276 | GCGGGGCATGTCGGGAGCGC | 63 |
| 277 | CGCGGGGCATGTCGGGAGCG | 62 |
| 278 | CCGCGGGGCATGTCGGGAGC | 61 |
| 279 | GCCGCGGGGCATGTCGGGAG | 60 |
| 280 | CGCCGCGGGGCATGTCGGGA | 59 |
| 281 | GCGCCGCGGGGCATGTCGGG | 58 |
| 282 | CGCGCCGCGGGGCATGTCGG | 57 |
| 283 | GCGCGCCGCGGGGCATGTCG | 56 |
| 284 | GGCGCGCCGCGGGGCATGTC | 55 |
| 285 | TGGCGCGCCGCGGGGCATGT | 54 |
| 286 | ATGGCGCGCCGCGGGGCATG | 53 |
| 287 | AATGGCGCGCCGCGGGGCAT | 52 |
| 288 | TAATGGCGCGCCGCGGGGCA | 51 |
| 289 | TTAATGGCGCGCCGCGGGGC | 50 |
| 290 | GTTAATGGCGCGCCGCGGGG | 49 |
| 291 | GGTTAATGGCGCGCCGCGGG | 48 |
| 292 | CGGTTAATGGCGCGCCGCGG | 47 |
| 293 | GCGGTTAATGGCGCGCCGCG | 46 |
| 294 | GGCGGTTAATGGCGCGCCGC | 45 |
| 295 | TGGCGGTTAATGGCGCGCCG | 44 |
| 296 | CTGGCGGTTAATGGCGCGCC | 43 |
| 297 | TCTGGCGGTTAATGGCGCGC | 42 |
| 298 | ATCTGGCGGTTAATGGCGCG | 41 |
| 299 | AATCTGGCGGTTAATGGCGC | 40 |
| 300 | AAATCTGGCGGTTAATGGCG | 39 |
| 301 | CAAATCTGGCGGTTAATGGC | 38 |
| 302 | TCAAATCTGGCGGTTAATGG | 37 |
| 303 | GTCGCCCCTGGGTCCTGCTGATTGGC | 1919 |
| 304 | TCGCCCCTGGGTCCTGCTGA | 1920 |
| 305 | CGCCCCTGGGTCCTGCTGAT | 1921 |
| 306 | GGTCGCCCCTGGGTCCTGCT | 1918 |
| 307 | AGGTCGCCCCTGGGTCCTGC | 1917 |
| 308 | CAGGTCGCCCCTGGGTCCTG | 1916 |
| 309 | GCAGGTCGCCCCTGGGTCCT | 1915 |
| 310 | GGCAGGTCGCCCCTGGGTCC | 1914 |
| 311 | TGGCAGGTCGCCCCTGGGTC | 1913 |
| 312 | TTGGCAGGTCGCCCCTGGGT | 1912 |
| 313 | TTTGGCAGGTCGCCCCTGGG | 1911 |
| 314 | CTTTGGCAGGTCGCCCCTGG | 1910 |
| 315 | ACTTTGGCAGGTCGCCCCTG | 1909 |
| 316 | GACTTTGGCAGGTCGCCCCT | 1908 |
| 317 | TGACTTTGGCAGGTCGCCCC | 1907 |
| 318 | TTGACTTTGGCAGGTCGCCC | 1906 |
| 319 | GTTGACTTTGGCAGGTCGCC | 1905 |
| 320 | AGTTGACTTTGGCAGGTCGC | 1904 |
| 321 | CAGTTGACTTTGGCAGGTCG | 1903 |
| 322 | CAGCGAGCCTGGGCCCCATCGGCACATCT | 2909 |
| 323 | AGCGAGCCTGGGCCCCATCG | 2910 |
| 324 | GCGAGCCTGGGCCCCATCGG | 2911 |
| 325 | CGAGCCTGGGCCCCATCGGC | 2912 |
| 326 | GAGCCTGGGCCCCATCGGCA | 2913 |
| 327 | AGCCTGGGCCCCATCGGCAC | 2914 |
| 328 | GCCTGGGCCCCATCGGCACA | 2915 |
| 329 | CCTGGGCCCCATCGGCACAT | 2916 |
| 330 | CTGGGCCCCATCGGCACATC | 2917 |
| 331 | TGGGCCCCATCGGCACATCT | 2918 |
| 332 | GGGCCCCATCGGCACATCTG | 2919 |
| 333 | GGCCCCATCGGCACATCTGA | 2920 |
| 334 | GCCCCATCGGCACATCTGAA | 2921 |
| 335 | CCCCATCGGCACATCTGAAG | 2922 |
| 336 | CCCATCGGCACATCTGAAGG | 2923 |
| 337 | CCATCGGCACATCTGAAGGT | 2924 |
| 338 | CATCGGCACATCTGAAGGTG | 2925 |
| 339 | ATCGGCACATCTGAAGGTGC | 2926 |
| 340 | TCGGCACATCTGAAGGTGCA | 2927 |
| 341 | CGGCACATCTGAAGGTGCAC | 2928 |
| 342 | GCAGCGAGCCTGGGCCCCAT | 2908 |
| 343 | TGCAGCGAGCCTGGGCCCCA | 2907 |
| 344 | CTGCAGCGAGCCTGGGCCCC | 2906 |
| 345 | TCTGCAGCGAGCCTGGGCCC | 2905 |
| 346 | ATCTGCAGCGAGCCTGGGCC | 2904 |
| 347 | CATCTGCAGCGAGCCTGGGC | 2903 |
| 348 | CCATCTGCAGCGAGCCTGGG | 2902 |
| 349 | GCCATCTGCAGCGAGCCTGG | 2901 |
| 350 | GGCCATCTGCAGCGAGCCTG | 2900 |
| 351 | GGGCCATCTGCAGCGAGCCT | 2899 |
| 352 | GGGGCCATCTGCAGCGAGCC | 2898 |
| 353 | GGGGGCCATCTGCAGCGAGC | 2897 |
| 354 | AGGGGGCCATCTGCAGCGAG | 2896 |
| 355 | AAGGGGGCCATCTGCAGCGA | 2895 |
| 356 | GAAGGGGGCCATCTGCAGCG | 2894 |
| 357 | CCCGCGGCCTTCTGGGAGTAGAGGC | 106 |
| 358 | CCGCGGCCTTCTGGGAGTAG | 107 |
| 359 | CGCGGCCTTCTGGGAGTAGA | 108 |
| 360 | GCGGCCTTCTGGGAGTAGAG | 109 |
| 361 | CGGCCTTCTGGGAGTAGAGG | 110 |
| 362 | CCCCGCGGCCTTCTGGGAGT | 105 |
| 363 | CCCCCGCGGCCTTCTGGGAG | 104 |
| 364 | CCCCCCGCGGCCTTCTGGGA | 103 |
| 365 | ACCCCCCGCGGCCTTCTGGG | 102 |
| 366 | CACCCCCCGCGGCCTTCTGG | 101 |
| 367 | CCACCCCCCGCGGCCTTCTG | 100 |
| 368 | TCCACCCCCCGCGGCCTTCT | 99 |
| 369 | GTCCACCCCCCGCGGCCTTC | 98 |
| 370 | GGTCCACCCCCCGCGGCCTT | 97 |
| 371 | CGGTCCACCCCCCGCGGCCT | 96 |
| 372 | GCGGTCCACCCCCCGCGGCC | 95 |
| 373 | GGCGGTCCACCCCCCGCGGC | 94 |
| 374 | AGGCGGTCCACCCCCCGCGG | 93 |
| 375 | TAGGCGGTCCACCCCCCGCG | 92 |
| 376 | TTAGGCGGTCCACCCCCCGC | 91 |
| 377 | CTTAGGCGGTCCACCCCCCG | 90 |
| 378 | TCTTAGGCGGTCCACCCCCC | 89 |
| 379 | CTCTTAGGCGGTCCACCCCC | 88 |
| 380 | CCTCTTAGGCGGTCCACCCC | 87 |
| 381 | CCCTCTTAGGCGGTCCACCC | 86 |
| 382 | GCCCTCTTAGGCGGTCCACC | 85 |
| 383 | CGCCCTCTTAGGCGGTCCAC | 84 |
| 384 | ACGCCCTCTTAGGCGGTCCA | 83 |
| 385 | CACGCCCTCTTAGGCGGTCC | 82 |
| 386 | GCACGCCCTCTTAGGCGGTC | 81 |
| 387 | CGCACGCCCTCTTAGGCGGT | 80 |
| 388 | GCGCACGCCCTCTTAGGCGG | 79 |
| 389 | AGCGCACGCCCTCTTAGGCG | 78 |
| 390 | GAGCGCACGCCCTCTTAGGC | 77 |
| 391 | GGAGCGCACGCCCTCTTAGG | 76 |
| 392 | GGGAGCGCACGCCCTCTTAG | 75 |
| 393 | CGGGAGCGCACGCCCTCTTA | 74 |
| 394 | TCGGGAGCGCACGCCCTCTT | 73 |
| 395 | GTCGGGAGCGCACGCCCTCT | 72 |
| 396 | TGTCGGGAGCGCACGCCCTC | 71 |
| 397 | ATGTCGGGAGCGCACGCCCT | 70 |
| 398 | CATGTCGGGAGCGCACGCCC | 69 |
| 399 | GCATGTCGGGAGCGCACGCC | 68 |
| 400 | GGCATGTCGGGAGCGCACGC | 67 |
| 401 | GGGCATGTCGGGAGCGCACG | 66 |
| 402 | GGGGCATGTCGGGAGCGCAC | 65 |
| 403 | CGGGGCATGTCGGGAGCGCA | 64 |
| 404 | GCGGGGCATGTCGGGAGCGC | 63 |
| 405 | CGCGGGGCATGTCGGGAGCG | 62 |
| 406 | CCGCGGGGCATGTCGGGAGC | 61 |
| 407 | GCCGCGGGGCATGTCGGGAG | 60 |
| 408 | CGCCGCGGGGCATGTCGGGA | 59 |
| 409 | GCGCCGCGGGGCATGTCGGG | 58 |
| 410 | CGCGCCGCGGGGCATGTCGG | 57 |
| 411 | GCGCGCCGCGGGGCATGTCG | 56 |
| 412 | GGCGCGCCGCGGGGCATGTC | 55 |
| 413 | TGGCGCGCCGCGGGGCATGT | 54 |
| 414 | ATGGCGCGCCGCGGGGCATG | 53 |
| 415 | AATGGCGCGCCGCGGGGCAT | 52 |
| 416 | TAATGGCGCGCCGCGGGGCA | 51 |
| 417 | TTAATGGCGCGCCGCGGGGC | 50 |
| 418 | GTTAATGGCGCGCCGCGGGG | 49 |
| 419 | GGTTAATGGCGCGCCGCGGG | 48 |
| 420 | CGGTTAATGGCGCGCCGCGG | 47 |
| 421 | GCGGTTAATGGCGCGCCGCG | 46 |
| 422 | GGCGGTTAATGGCGCGCCGC | 45 |
| 423 | TGGCGGTTAATGGCGCGCCG | 44 |
| 424 | CTGGCGGTTAATGGCGCGCC | 43 |
| 425 | TCTGGCGGTTAATGGCGCGC | 42 |
| 426 | ATCTGGCGGTTAATGGCGCG | 41 |
| 427 | AATCTGGCGGTTAATGGCGC | 40 |
| 428 | AAATCTGGCGGTTAATGGCG | 39 |
| 429 | CAAATCTGGCGGTTAATGGC | 38 |
| 430 | TCAAATCTGGCGGTTAATGG | 37 |
| 431 | TCCCGGCGAGTACATCGTTGACTGCACG | 679 |
| 432 | CCCGGCGAGTACATCGTTGA | 680 |
| 433 | CCGGCGAGTACATCGTTGAC | 681 |
| 434 | CGGCGAGTACATCGTTGACT | 682 |
| 435 | GGCGAGTACATCGTTGACTG | 683 |
| 436 | GCGAGTACATCGTTGACTGC | 684 |
| 437 | CGAGTACATCGTTGACTGCA | 685 |
| 438 | GAGTACATCGTTGACTGCAC | 686 |
| 439 | AGTACATCGTTGACTGCACG | 687 |
| 440 | GTACATCGTTGACTGCACGA | 688 |
| 441 | TACATCGTTGACTGCACGAC | 689 |
| 442 | ACATCGTTGACTGCACGACC | 690 |
| 443 | CATCGTTGACTGCACGACCT | 691 |
| 444 | ATCGTTGACTGCACGACCTG | 692 |
| 445 | TCGTTGACTGCACGACCTGG | 693 |
| 446 | CGTTGACTGCACGACCTGGG | 694 |
| 447 | GTTGACTGCACGACCTGGGT | 695 |
| 448 | TTGACTGCACGACCTGGGTT | 696 |
| 449 | TGACTGCACGACCTGGGTTT | 697 |
| 450 | GACTGCACGACCTGGGTTTC | 698 |
| 451 | ACTGCACGACCTGGGTTTCC | 699 |
| 452 | CTGCACGACCTGGGTTTCCA | 700 |
| 453 | TGCACGACCTGGGTTTCCAG | 701 |
| 454 | GCACGACCTGGGTTTCCAGG | 702 |
| 455 | CACGACCTGGGTTTCCAGGA | 703 |
| 456 | ACGACCTGGGTTTCCAGGAG | 704 |
| 457 | CGACCTGGGTTTCCAGGAGG | 705 |
| 458 | GTCCCGGCGAGTACATCGTT | 678 |
| 459 | TGTCCCGGCGAGTACATCGT | 677 |
| 460 | CTGTCCCGGCGAGTACATCG | 676 |
| 461 | GCTGTCCCGGCGAGTACATC | 675 |
| 462 | CGCTGTCCCGGCGAGTACAT | 674 |
| 463 | TCGCTGTCCCGGCGAGTACA | 673 |
| 464 | ATCGCTGTCCCGGCGAGTAC | 672 |
| 465 | CATCGCTGTCCCGGCGAGTA | 671 |
| 466 | ACATCGCTGTCCCGGCGAGT | 670 |
| 467 | GACATCGCTGTCCCGGCGAG | 669 |
| 468 | AGACATCGCTGTCCCGGCGA | 668 |
| 469 | CAGACATCGCTGTCCCGGCG | 667 |
| 470 | GCAGACATCGCTGTCCCGGC | 666 |
| 471 | AGCAGACATCGCTGTCCCGG | 665 |
| 472 | CAGCAGACATCGCTGTCCCG | 664 |
| 473 | GCAGCAGACATCGCTGTCCC | 663 |
| 474 | TGCAGCAGACATCGCTGTCC | 662 |
| 475 | GTGCAGCAGACATCGCTGTC | 661 |
| 476 | AGTGCAGCAGACATCGCTGT | 660 |
| 477 | GAGTGCAGCAGACATCGCTG | 659 |
| 478 | GGAGTGCAGCAGACATCGCT | 658 |
| 479 | TGGAGTGCAGCAGACATCGC | 657 |
| 480 | ATGGAGTGCAGCAGACATCG | 656 |
| 481 | AACCTCCTCCCCGCCACGGGTT | 1233 |
| 482 | ACCTCCTCCCCGCCACGGGT | 1234 |
| 483 | CCTCCTCCCCGCCACGGGTT | 1235 |
| 484 | CTCCTCCCCGCCACGGGTTC | 1236 |
| 485 | TCCTCCCCGCCACGGGTTCA | 1237 |
| 486 | CCTCCCCGCCACGGGTTCAA | 1238 |
| 487 | CTCCCCGCCACGGGTTCAAG | 1239 |
| 488 | TCCCCGCCACGGGTTCAAGC | 1240 |
| 489 | CCCCGCCACGGGTTCAAGCG | 1241 |
| 490 | CCCGCCACGGGTTCAAGCGA | 1242 |
| 491 | CCGCCACGGGTTCAAGCGAT | 1243 |
| 492 | CGCCACGGGTTCAAGCGATT | 1244 |
| 493 | GCCACGGGTTCAAGCGATTC | 1245 |
| 494 | CCACGGGTTCAAGCGATTCT | 1246 |
| 495 | CACGGGTTCAAGCGATTCTC | 1247 |
| 496 | ACGGGTTCAAGCGATTCTCC | 1248 |
| 497 | CGGGTTCAAGCGATTCTCCT | 1249 |
| 498 | GGGTTCAAGCGATTCTCCTG | 1250 |
| 499 | GGTTCAAGCGATTCTCCTGC | 1251 |
| 500 | GTTCAAGCGATTCTCCTGCC | 1252 |
| 501 | TTCAAGCGATTCTCCTGCCT | 1253 |
| 502 | TCAAGCGATTCTCCTGCCTC | 1254 |
| 503 | CAAGCGATTCTCCTGCCTCA | 1255 |
| 504 | AAGCGATTCTCCTGCCTCAG | 1256 |
| 505 | AGCGATTCTCCTGCCTCAGC | 1257 |
| 506 | GCGATTCTCCTGCCTCAGCC | 1258 |
| 507 | CGATTCTCCTGCCTCAGCCT | 1259 |
| 508 | CAACCTCCTCCCCGCCACGG | 1232 |
| 509 | GCAACCTCCTCCCCGCCACG | 1231 |
| 510 | TGCAACCTCCTCCCCGCCAC | 1230 |
| 511 | CTGCAACCTCCTCCCCGCCA | 1229 |
| 512 | ACTGCAACCTCCTCCCCGCC | 1228 |
| 513 | CACTGCAACCTCCTCCCCGC | 1227 |
| 514 | TCACTGCAACCTCCTCCCCG | 1226 |

Hot Zones (Relative upstream location to gene start site)
1-350
600-800
1100-1350
1900-2150
2750-3200

### Examples

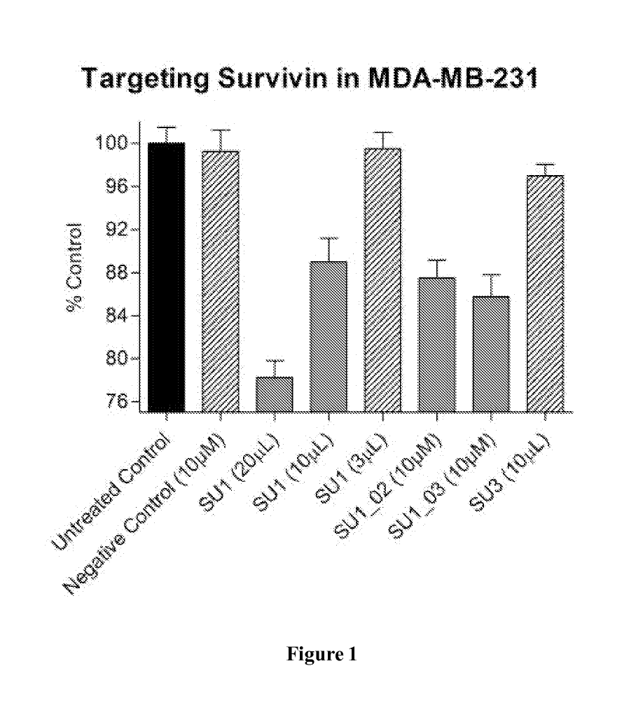

In FIG. 1, SU1 (1) shows a dose-dependent response in MDA-MB-231, a human breast cell line, with SU1 at 20 μL showing greater inhibition than SU1 at 10 and 3 μM. SU1's inhibition values, both at 20 and 10 μM, were statistically significant (P<0.05) compared to untreated control values. SU1's inhibition values at 3 μM were insignificant (insignificance indicated by bars with diagonal stripes). Furthermore, SU3's (3) inhibition values at 10 μM were insignificant compared to the untreated control values. SU3's diminished inhibition is attributable to the lack of a CG pair in the 5′ linear section before or at the base of the hairpin of the secondary structure and further back from the transcription start site compared to the other oligonucleotides tested. Two variants of SU1, SU1_{—}02 (4; 1 base shorter) and SU1_{—}03 (5; 4 bases longer), were also statistically significant at 10 μM (P<0.5) compared to the untreated control. This demonstrates that a sequence still retains its inhibitory levels despite shifting the sequence a few bases. The negative control (a scrambled oligonucleotide) was not statistically significant compared to the untreated control. The Survivin sequences SU1 (1), SU1_{—}02 (4), SU1_{—}03 (5) (shown below) fit the independent and dependent DNAi motif claims. As noted previously, SU3 (3), does not contain a CG in the 5′ linear section either prior to or in the base of the hairpin.

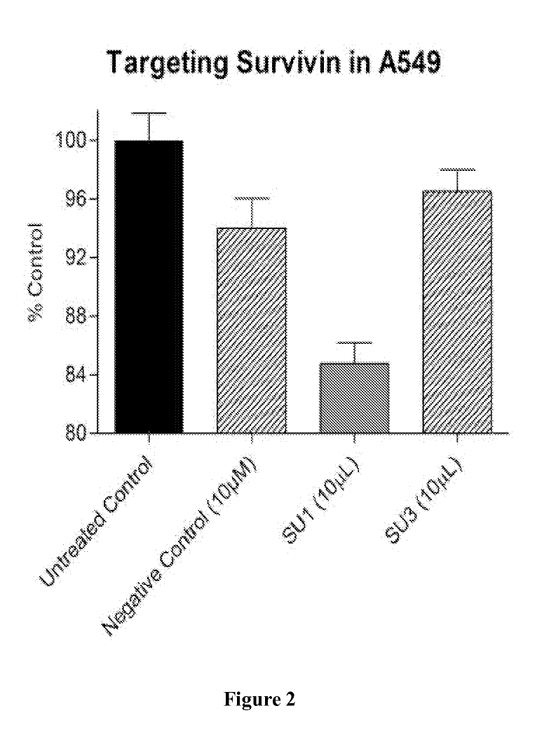

FIG. 2 is similar to FIG. 1 and in FIG. 2 it is shown that SU1 (1) demonstrated significant (P<0.05) inhibition of A549 (human lung cell line) compared to the untreated control values. Also, SU3's (3) inhibition values were insignificant compared to the untreated control values. The negative control was not statistically significant compared to the untreated control. The Survivin sequence SU1 (1) (shown below) fits the independent and dependent DNAi motif claims. As noted previously, SU3 (3), does not contain a CG in the 5′ linear section either prior to or in the base of the hairpin.

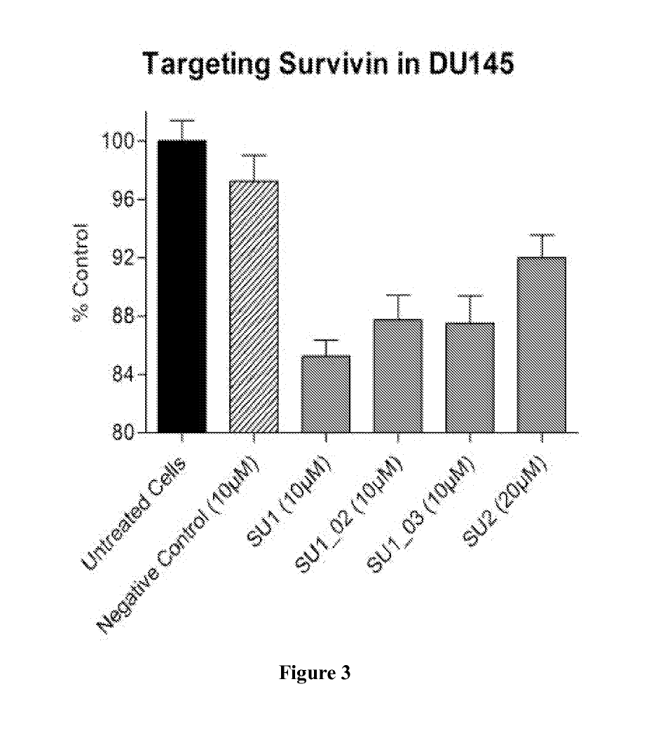

FIG. 3 shows that DU145 (human prostate cell line), SU1 (1) and its two variants, SU1_{—}02 (4) and SU1_{—}03 (5), produced statistically significant (P<0.05) inhibition at 10 μM compared to the untreated control values. SU2 (2), at 20 μM, produced statistically significant (P<0.05) inhibition compared to the untreated control values. The Survivin sequences SU1 (1), SU1_{—}02 (4), SU1_{—}03 (5), and SU2 (2) (shown below) fit the independent and dependent DNAi motif claims. As noted previously, SU3 (3), does not contain a CG in the 5′ linear section either prior to or in the base of the hairpin. SU2 (2) demonstrates that some oligonucleotides will show inhibition at acceptably higher concentrations (below a concentration where general cytotoxicity is observed) even though they may not demonstrate inhibition at lower concentrations.

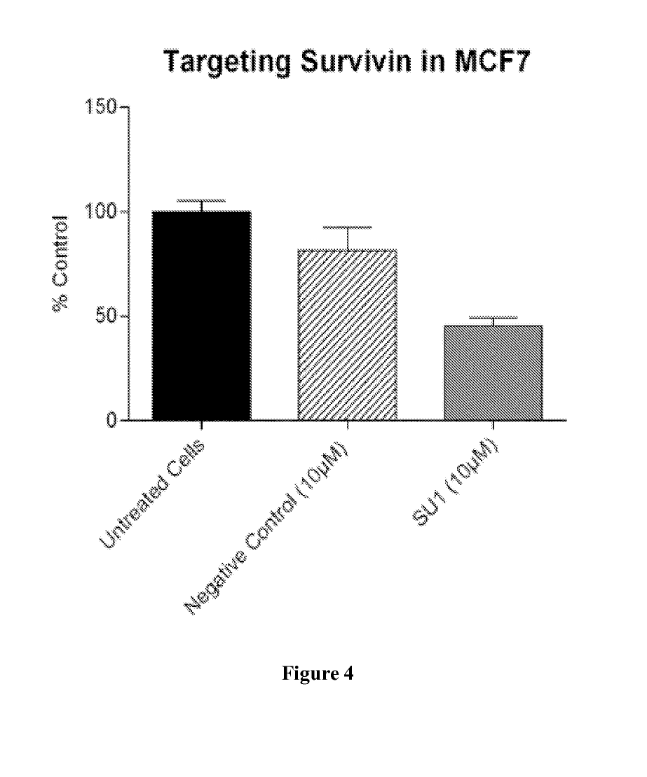

FIG. 4 shows that in MCF7 (human mammary breast cell line), SU1 (1) produced statistically significant (P<0.05) inhibition at 10 μM compared to the untreated and negative control values. The Survivin sequence SU1 (1), fits the independent and dependent DNAi motif claims.

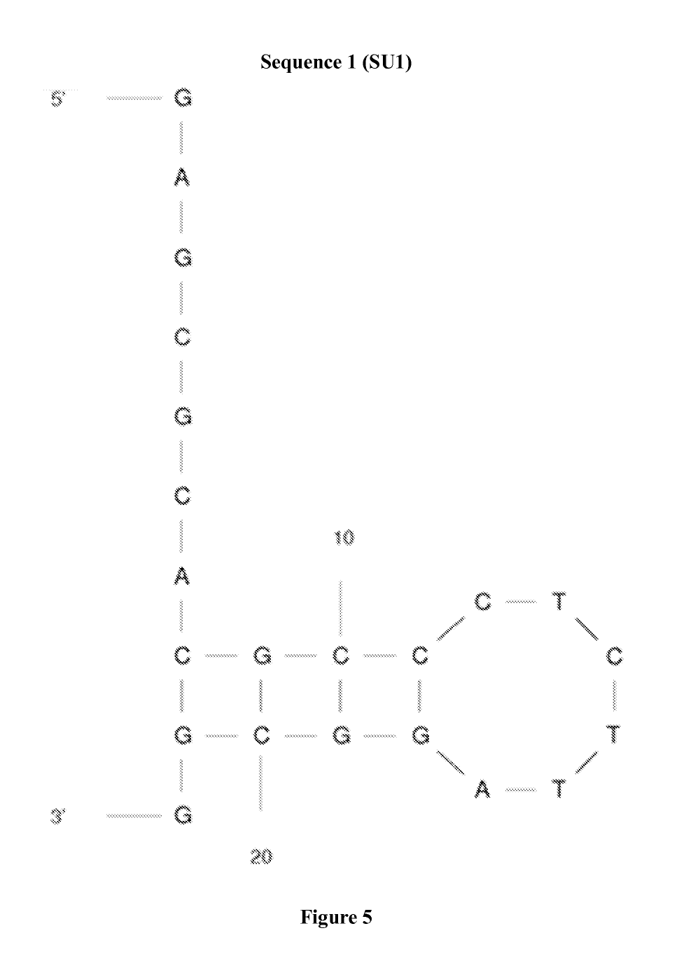

Secondary Structures FIGS. 5, 6, 7, 8, 9.

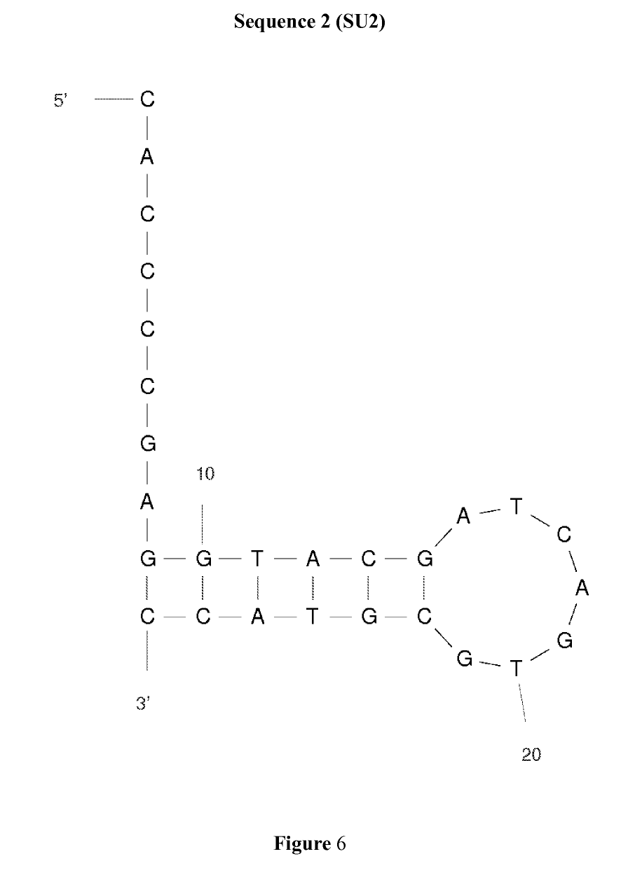

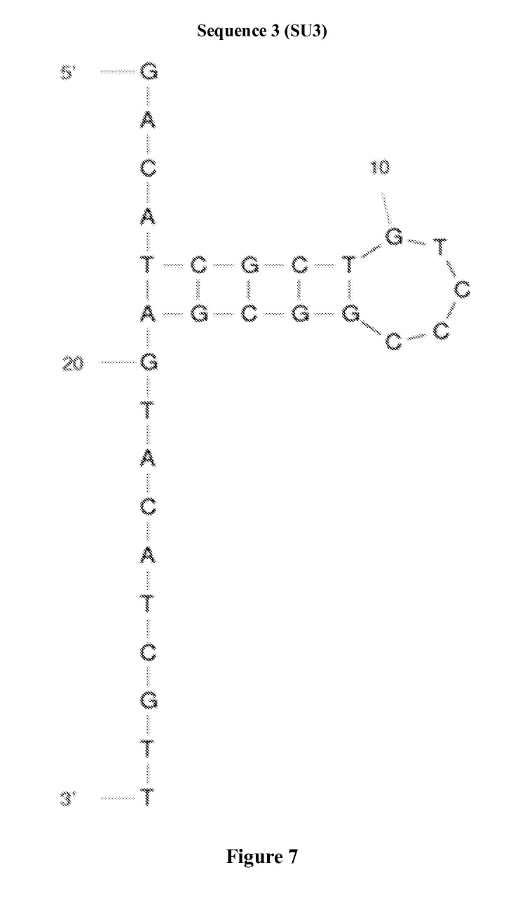

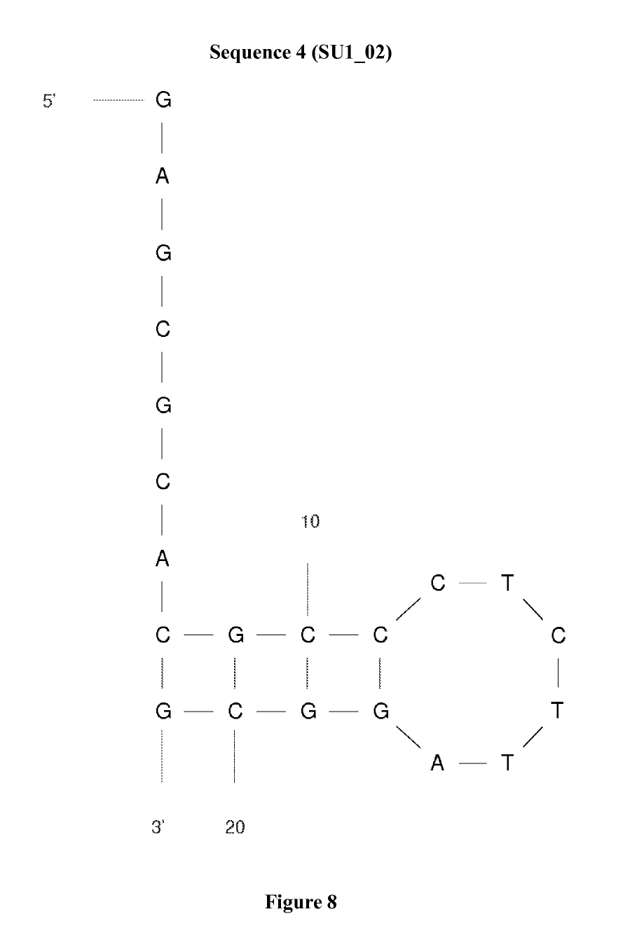

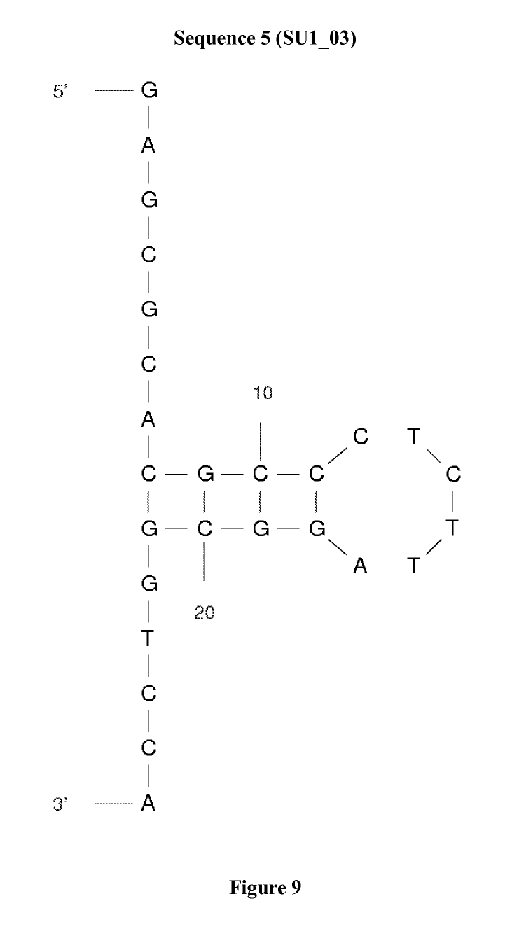

FIG. 5 is Sequence 1 (SU1). FIG. 6 is Sequence 2 (SU2). FIG. 7 is Sequence 3 (SU3) (Note in FIG. 7 or Sequence 3 there is No CG in the 5′ linear base. FIG. 8 is Sequence 4 (SU1_{—}02). FIG. 9 is Sequence 5 (SU1_{—}03).

Genetic Code (5′ Upstream Region)

(SEQ ID NO: 11950)

ATCATGACACACATTTACCTGTGTAACAAACCTGCACATCCTACACA
TATACCCTGGAACTTAAAGTAAAAGTTGGGGGGGGGGGTAAAAAAGAATT
TCCACCGTGACATTATTGAGTATAGCAAAAAAAAAAAAAACAAGAAACAG
CCTAGTGTTCATTAGGGAATAAACGCATTCAAGCAGCATCAAACCCTGCA
GCCATTACAAAGAGATCTATGTTGACCATGTGGAATATCTCCAAGAGCCA
CAGTAGCCTCCCTTATCTGTAGGATTCACTCCAAGACCCTCTGAAACCAT
GGATAATACTGAACCCTATATACACTATGTTTTTTCTTGTATATACATAC
CTACGATAAAGTTTAATTTATAAATTGGCAAAGGGTATATAAATATTCCT
TCTAAGAGATTAACAATAACTAATAAAGTAGAACGATTAAAACAATATAC
TGTGATCAAAGTTATGTGAAGCCAGGTGCTGTGGCTCATGCCTGTAATCC
CAGCACTTTGGGAGGCTGAGACAGGTGGATCACCTGAGGTCAGGAGTTGG
AGACCAGCCTGGCCAACATGACAAAACCCCGTCTCTACTAAAGATAAAAA
AAATTAGCCGGGCATGGTGACACATGCCTGTAATCCCAGCTACTTGGGAG
GCTGAGGCAGGAGAATCGCTTGAACCTGGGAGGCGGAGGTTGCAGTGAGC
TAAGATCACACCATTGCACTCCAGCCTGGGCAACAAGAGTGAAACTCTGT
CTCAAAACAAAACAAAACAAAACAAACTTATGGGGTTGCTCTCTTTCTCT
CAAAATATCCTTTTTTTGGCAGGGCACGGTGGCTCATGCCTGTAATCCCA
GCACTTTGAGAGGCTGAGGTGGGTGAATCACCTGAGGTCAGGAGTTCAAG
ACCAGCCTGGCCAACATGGTGAAACCCCGTCTCTATTAAAAATACAAAAA
ATTAGCTGGGCGTGGTGGTGCAGGCCTGTAATCCCAGCTACTTGGGAGGC
TGAGGCAGGAGAATCACTCGAACCCAGGAGCTGGAGTTTGCAGTGAGCCG
AGATCATGCCATTGCACTCCAGCCTGGGCCACAGAGCAAGACTCCATCTC
AAAAAAAAAAAAAAGAAAAAAAGAAAGTCTTTTTTTTTTTTGAGACTGTA
TCTCACTCTTTCTCCCAGGCTGGAGTGCAGTGGCCCAATCATGGCTCACT
GCAGCCTCGACCTCCCAGGATCAAGTGATCCTTCCACCTCAGCCTCCCGA
GTAGCTGGAAGTATAGGTGCACGCCCGACTGATTTTTTTTTTTTTTTTTA
GACGGAGTCTCACTCTTGTTGCTCTGGCTGGAGTGCAATGGCAGGATCTC
GGCTCACTGCAACCTCTGCCTCTTAGATTCAAGCGATTCTCGTGCCTCAG
CCTCCCGAGTAGCTGGGATTACAGGTGCCCACCACCATGCCCGGATAATT
TTTTGTATTTTTAATAGAGACAGGGTTTCACCATATTGGTCAGGCTGGTC
TCAAACTCCTGACCTCAGGTGATCCACCTGCCTCAGCCTCCCAAACTGCT
GGGATTACAGGCGTGAGCCACCGGGCATGGCCTTTCCTGGCTAATTTTTT
AAATTTTTGATAGAGATGGGGTCTCAGTGTTGCCCAGGCTGATCTTGAAC
TCCTAGATTCAAGTGATCCTCCCTCCTTGGTCTCCCAAAGTGCTGAGATT
ACAGGCGTGAGCCACCGCCCCGGGCTGGAAAATACTTTTTTAAACGAGGG
CAATGTGAATCTGAAATGCCATTTGAGGAAAGATCTGTTCGCCTGACATC
CTGTTTGAGCCTGGGTGGACAGGACAGCACCTGCCAGCATCGGGAAGCAC
TGCAGATGGGAAGAGGCTTGGTCACTCTCCAAAGGTGGCAGGAGTTGGAG
GGGGTGAGCTGAAGGTAAGGAGAAAGGAGGTGGGGACCCAGGAGACAGGG
GCTGCGCAGCGGGCTCGGGGCTGACACCCCCACGGATACAGTTCACTGGG
GCTCAAACATAAAAGGAACCCAACTATTGTGGGAGGAAAAGACTCTTCTG
CCTTTCTGCCTTTTCTTTTTTTCTTTTTCTTTCTTTCTTTTTTTTTTTTT
TTTTTTGAGACAGAGTCTTGCTCTATCGCCCAGGCTGGAGTGCAGTGGCG
TGATCTCGGCTCACTGCAAGCTCTGCCTCCCGGGATCACGCCATTCTCCT
GCCTCAACCTCCCGAGCAGCTGGGACTACAGGCGCCTGCCACCACACCCG
GCTATTTTTTTGTATTTTTTAGTAGAGATGGGGTTTCACCGTGTTAGCCA
GGACGGTCTCGATCTCCTGACCTTGTGATCCGCCCGCCTCGGCCTCCCAA
AGTGCTGGGATTACAGGCGTGAGCCACCGCGCCTGGCTCTTTTTTCTTTC
TTTTTTTTTTTTCCGAGACAGAGTTTCACTCTTGTTGCCCAGGCTGGAGT
GCAGTGGCGCAATCTTGGCTCACTGCAACCTCCACCTCCAGGGTTCAAGC
GATTCTCCTGCCTCAGCCTCCTGAGTAGCTGGGACTGCAGGCGCGCACCA
CCACGCCTGGCTAATTTTTGTATTTTTAGTAGAGACAGGGTTTCACCATA
TTGGCCAGGCTGGTCTCGAACTCCTGACCTTGTGATCTGCCCACCTCAGC
CTCCCAAAGTCCTGGGATTACAGGCGTGAGCCACCGTGCCCAGCCTGACC
CCTCTGCCCTTTCAAAAACTATGTTCGTTCTCTCACAGCCTTCTCTTGTC
ATATTAAGTCCACACCGCAGGCCTAATTTGTCCAGTGAATGCTATGCAAA
TATTTCATGCACCTGCTGATCGCAGGAATGATATGTACTTGGTACGCACT
GATCGTACCTCGGGGTGGGAGAAGAGAGGGCAAGGAAGCAAAGAATAGCC
CCCTCCTTTCCTGGTGCACCTTCAGATGTGCCGATGGGGCCCAGGCTCGC
TGCAGATGGCCCCCTTCCCAGAGACAGGGGAGGATCCTCCACCCACTCCC
CAGCCTCCAGGACCATCCTGACTCCTGCCTTCAGGCACTCAAGTTATGCG
TCTAGACATGCGGATATATTCAAGCTGGGCACAGCACAGCAGCCCCACCC
CAGGCAGCTTGAAATCAGAGCTGGGGTCCAAAGGGACCACACCCCGAGGG
ACTGTGTGGGGGTCGGGGCACACAGGCCACTGCTTCCCCCCGTCTTTCTC
AGCCATTCCTGAAGTCAGCCTCACTCTGCTTCTCAGGGATTTCAAATGTG
CAGAGACTCTGGCACTTTTGTAGAAGCCCCTTCTGGTCCTAACTTACACC
TGGATGCTGTGGGGCTGCAGCTGCTGCTCGGGCTCGGGAGGATGCTGGGG
GCCCGGTGCCCATGAGCTTTTGAAGCTCCTGGAACTCGGTTTTGAGGGTG
TTCAGGTCCAGGTGGACACCTGGGCTGTCCTTGTCCATGCATTTGATGAC
ATTGTGTGCAGAAGTGAAAAGGAGTTAGGCCGGGCATGCTGGCTTATGCC
TGTAATCCCAGCACTTTGGGAGGCTGAGGCGGGTGGATCACGAGGTCAGG
AGTTCAATACCAGCCTGGCCAAGATGGTGAAACCCCGTCTCTACTAAAAA
TACAAAAAAATTAGCCGGGCATGGTGGCGGGCGCATGTAATCCCAGCTAC
TGGGGGGGCTGAGGCAGAGAATTGCTGGAACCCAGGAGATGGAGGTTGCA
GTGAGCCAAGATTGTGCCACTGCACTGCACTCCAGCCTGGCGACAGAGCA
AGACTCTGTCTCAAAAAAAAAAAAAAAAAGTGAAAAGGAGTTGTTCCTTT
CCTCCCTCCTGAGGGCAGGCAACTGCTGCGGTTGCCAGTGGAGGTGGTGC
GTCCTTGGTCTGTGCCTGGGGGCCACCCCAGCAGAGGCCATGGTGGTGCC
AGGGCCCGGTTAGCGAGCCAATCAGCAGGACCCAGGGGCGACCTGCCAAA
GTCAACTGGATTTGATAACTGCAGCGAAGTTAAGTTTCCTGATTTTGATG
ATTGTGTTGTGGTTGTGTAAGAGAATGAAGTATTTCGGGGTAGTATGGTA
ATGCCTTCAACTTACAAACGGTTCAGGTAAACCACCCATATACATACATA
TACATGCATGTGATATATACACATACAGGGATGTGTGTGTGTTCACATAT
ATGAGGGGAGAGAGACTAGGGGAGAGAAAGTAGGTTGGGGAGAGGGAGAG
AGAAAGGAAAACAGGAGACAGAGAGAGAGCGGGGAGTAGAGAGAGGGAAG
GGGTAAGAGAGGGAGAGGAGGAGAGAAAGGGAGGAAGAAGCAGAGAGTGA
ATGTTAAAGGAAACAGGCAAAACATAAACAGAAAATCTGGGTGAAGGGTA
TATGAGTATTCTTTGTACTATTCTTGCAATTATCTTTTATTTAAATTGAC
ATCGGGCCGGGCGCAGTGGCTCACATCTGTAATCCCAGCACTTTGGGAGG
CCGAGGCAGGCAGATCACTTGAGGTCAGGAGTTTGAGACCAGCCTGGCAA
ACATGGTGAAACCCCATCTCTACTAAAAATACAAAAATTAGCCTGGTGTG
GTGGTGCATGCCTTTAATCTCAGCTACTCGGGAGGCTGAGGCAGGAGAAT
CGCTTGAACCCGTGGCGGGGAGGAGGTTGCAGTGAGCTGAGATCATGCCA
CTGCACTCCAGCCTGGGCGATAGAGCGAGACTCAGTTTCAAATAAATAAA
TAAACATCAAAATAAAAAGTTACTGTATTAAAGAATGGGGGCGGGGTGGG
AGGGGTGGGGAGAGGTTGCAAAAATAAATAAATAAATAAATAAACCCCAA
AATGAAAAAGACAGTGGAGGCACCAGGCCTGCGTGGGGCTGGAGGGCTAA
TAAGGCCAGGCCTCTTATCTCTGGCCATAGAACCAGAGAAGTGAGTGGAT
GTGATGCCCAGCTCCAGAAGTGACTCCAGAACACCCTGTTCCAAAGCAGA
GGACACACTGATTTTTTTTTTAATAGGCTGCAGGACTTACTGTTGGTGGG
ACGCCCTGCTTTGCGAAGGGAAAGGAGGAGTTTGCCCTGAGCACAGGCCC
CCACCCTCCACTGGGCTTTCCCCAGCTCCCTTGTCTTCTTATCACGGTAG
TGGCCCAGTCCCTGGCCCCTGACTCCAGAAGGTGGCCCTCCTGGAAACCC
AGGTCGTGCAGTCAACGATGTACTCGCCGGGACAGCGATGTCTGCTGCAC
TCCATCCCTCCCCTGTTCATTTGTCCTTCATGCCCGTCTGGAGTAGATGC
TTTTTGCAGAGGTGGCACCCTGTAAAGCTCTCCTGTCTGACTTTTTTTTT
TTTTTTAGACTGAGTTTTGCTCTTGTTGCCTAGGCTGGAGTGCAATGGCA
CAATCTCAGCTCACTGCACCCTCTGCCTCCCGGGTTCAAGCGATTCTCCT
GCCTCAGCCTCCCGAGTAGTTGGGATTACAGGCATGCACCACCACGCCCA
GCTAATTTTTGTATTTTTAGTAGAGACAAGGTTTCACCGTGATGGCCAGG
CTGGTCTTGAACTCCAGGACTCAAGTGATGCTCCTGCCTAGGCCTCTCAA
AGTGTTGGGATTACAGGCGTGAGCCACTGCACCCGGCCTGCACGCGTTCT
TTGAAAGCAGTCGAGGGGGCGCTAGGTGTGGGCAGGGACGAGCTGGCGCG
GCGTCGCTGGGTGCACCGCGACCACGGGCAGAGCCACGCGGCGGGAGGAC
TACAACTCCCGGCACACCCCGCGCCGCCCCGCCTCTACTCCCAGAAGGCC
GCGGGGGGTGGACCGCCTAAGAGGGCGTGCGCTCCCGACATGCCCCGCGG
CGCGCCATTAACCGCCAGATTTGAATCGCGGGACCCGTTGGCAGAGGTGG
CGGCGGCGGCATG

2) Beclin-1. Beclin 1, the mammalian orthologue of yeast Atg6, has a central role in autophagy, a process of programmed cell survival, which is increased during periods of cell stress and extinguished during the cell cycle. It interacts with several cofactors (Atg14L, UVRAG, Bif-1, Rubicon, Ambra1, HMGB1, nPIST, VMP1, SLAM, IP₃R, PINK and survivin) to regulate the lipid kinase Vps-34 protein and promote formation of Beclin 1-Vps34-Vps15 core complexes, thereby inducing autophagy. In contrast, the BH3 domain of Beclin 1 is bound to, and inhibited by Bcl-2 or Bcl-XL. This interaction can be disrupted by phosphorylation of Bcl-2 and Beclin 1, or ubiquitination of Beclin 1. Interestingly, caspase-mediated cleavage of Beclin 1 promotes crosstalk between apoptosis and autophagy. Beclin 1 dysfunction has been implicated in many disorders, including cancer and neurodegeneration (reviewed by Kang et al., Cell Death Differ. 2011 April; 18(4): 571-580).

Protein: Beclin-1 Gene: BECN1 (Homo sapiens, chromosome 17, 40962150-40976310 [NCBI Reference Sequence: NC_{—}000017.10]; start site location: 40975895; strand: negative)

Gene Identification

| | |
|---|---|
| GeneID | 8678 |
| HGNC | 1034 |
| HPRD | 05087 |
| MIM | 604378 |

Targeted Sequences

| | | | Relative |
|---|---|---|---|
| | | | upstream |
| | | | location |
| Se- | De- | | to gene |
| quence | sign | | start |
| ID No: | ID | Sequence (5′-3′) | site |
| 515 | BE1 | CGACGCCCTTGACCTCCGGCCCGGGGT | 39 |
| 550 | BE2 | CTGCGCCGTTCCCTCTAGGAATGG | 111 |
| 572 | | GAAGCGACGCCCTTGACCTCCGGCCCGG | 35 |
| 607 | | CCCCCGATGCTCTTCACCTCGGG | 261 |
| 712 | | CGGGTCGGCCCCGGAGCGAGGCC | 335 |
| 817 | | GCCCGGCAGCGGCCCCCAGAGGCCG | 475 |
| 847 | | CGGTCTACCGCGGAGGCACTGTGGCCTCGG | 308 |
| 952 | | ACAAAAACTAGCCGGGCGTGGTGGGGCACG | 735 |
| | | CC | |

Target Shift Sequences

| | | Relative |
|---|---|---|
| | | upstream |
| | | location |
| | | to gene |
| Sequence | | start |
| ID No: | Sequence (5′-3′) | site |
| 515 | CGACGCCCTTGACCTCCGGCCCGGGGT | 39 |
| 516 | GACGCCCTTGACCTCCGGCC | 40 |
| 517 | ACGCCCTTGACCTCCGGCCC | 41 |
| 518 | CGCCCTTGACCTCCGGCCCG | 42 |
| 519 | GCCCTTGACCTCCGGCCCGG | 43 |
| 520 | CCCTTGACCTCCGGCCCGGG | 44 |
| 521 | CCTTGACCTCCGGCCCGGGG | 45 |
| 522 | CTTGACCTCCGGCCCGGGGT | 46 |
| 523 | TTGACCTCCGGCCCGGGGTT | 47 |
| 524 | TGACCTCCGGCCCGGGGTTA | 48 |
| 525 | GACCTCCGGCCCGGGGTTAC | 49 |
| 526 | ACCTCCGGCCCGGGGTTACC | 50 |
| 527 | CCTCCGGCCCGGGGTTACCA | 51 |
| 528 | CTCCGGCCCGGGGTTACCAC | 52 |
| 529 | TCCGGCCCGGGGTTACCACA | 53 |
| 530 | CCGGCCCGGGGTTACCACAT | 54 |
| 531 | CGGCCCGGGGTTACCACATG | 55 |
| 532 | GGCCCGGGGTTACCACATGC | 56 |
| 533 | GCCCGGGGTTACCACATGCC | 57 |
| 534 | CCCGGGGTTACCACATGCCT | 58 |
| 535 | CCGGGGTTACCACATGCCTT | 59 |
| 536 | CGGGGTTACCACATGCCTTG | 60 |
| 537 | GCGACGCCCTTGACCTCCGG | 38 |
| 538 | AGCGACGCCCTTGACCTCCG | 37 |
| 539 | AAGCGACGCCCTTGACCTCC | 36 |
| 540 | GAAGCGACGCCCTTGACCTC | 35 |
| 541 | AGAAGCGACGCCCTTGACCT | 34 |
| 542 | GAGAAGCGACGCCCTTGACC | 33 |
| 543 | GGAGAAGCGACGCCCTTGAC | 32 |
| 544 | GGGAGAAGCGACGCCCTTGA | 31 |
| 545 | AGGGAGAAGCGACGCCCTTG | 30 |
| 546 | TAGGGAGAAGCGACGCCCTT | 29 |
| 547 | TTAGGGAGAAGCGACGCCCT | 28 |
| 548 | ATTAGGGAGAAGCGACGCCC | 27 |
| 549 | CATTAGGGAGAAGCGACGCC | 26 |
| 550 | CTGCGCCGTTCCCTCTAGGAATGG | 111 |
| 551 | TGCGCCGTTCCCTCTAGGAA | 112 |
| 552 | GCGCCGTTCCCTCTAGGAAT | 113 |
| 553 | CGCCGTTCCCTCTAGGAATG | 114 |
| 554 | GCCGTTCCCTCTAGGAATGG | 115 |
| 555 | CCGTTCCCTCTAGGAATGGT | 116 |
| 556 | CGTTCCCTCTAGGAATGGTA | 117 |
| 557 | CCTGCGCCGTTCCCTCTAGG | 110 |
| 558 | ACCTGCGCCGTTCCCTCTAG | 109 |
| 559 | AACCTGCGCCGTTCCCTCTA | 108 |
| 560 | CAACCTGCGCCGTTCCCTCT | 107 |
| 561 | CCAACCTGCGCCGTTCCCTC | 106 |
| 562 | CCCAACCTGCGCCGTTCCCT | 105 |
| 563 | TCCCAACCTGCGCCGTTCCC | 104 |
| 564 | GTCCCAACCTGCGCCGTTCC | 103 |
| 565 | AGTCCCAACCTGCGCCGTTC | 102 |
| 566 | AAGTCCCAACCTGCGCCGTT | 101 |
| 567 | GAAGTCCCAACCTGCGCCGT | 100 |
| 568 | GGAAGTCCCAACCTGCGCCG | 99 |
| 569 | GGGAAGTCCCAACCTGCGCC | 98 |
| 570 | AGGGAAGTCCCAACCTGCGC | 97 |
| 571 | GAGGGAAGTCCCAACCTGCG | 96 |
| 572 | GAAGCGACGCCCTTGACCTCCGGCCCGG | 35 |
| 573 | AAGCGACGCCCTTGACCTCC | 36 |
| 574 | AGCGACGCCCTTGACCTCCG | 37 |
| 575 | GCGACGCCCTTGACCTCCGG | 38 |
| 576 | CGACGCCCTTGACCTCCGGC | 39 |
| 577 | GACGCCCTTGACCTCCGGCC | 40 |
| 578 | ACGCCCTTGACCTCCGGCCC | 41 |
| 579 | CGCCCTTGACCTCCGGCCCG | 42 |
| 580 | GCCCTTGACCTCCGGCCCGG | 43 |
| 581 | CCCTTGACCTCCGGCCCGGG | 44 |
| 582 | CCTTGACCTCCGGCCCGGGG | 45 |
| 583 | CTTGACCTCCGGCCCGGGGT | 46 |
| 584 | TTGACCTCCGGCCCGGGGTT | 47 |
| 585 | TGACCTCCGGCCCGGGGTTA | 48 |
| 586 | GACCTCCGGCCCGGGGTTAC | 49 |
| 587 | ACCTCCGGCCCGGGGTTACC | 50 |
| 588 | CCTCCGGCCCGGGGTTACCA | 51 |
| 589 | CTCCGGCCCGGGGTTACCAC | 52 |
| 590 | TCCGGCCCGGGGTTACCACA | 53 |
| 591 | CCGGCCCGGGGTTACCACAT | 54 |
| 592 | CGGCCCGGGGTTACCACATG | 55 |
| 593 | GGCCCGGGGTTACCACATGC | 56 |
| 594 | GCCCGGGGTTACCACATGCC | 57 |
| 595 | CCCGGGGTTACCACATGCCT | 58 |
| 596 | CCGGGGTTACCACATGCCTT | 59 |
| 597 | CGGGGTTACCACATGCCTTG | 60 |
| 598 | AGAAGCGACGCCCTTGACCT | 34 |
| 599 | GAGAAGCGACGCCCTTGACC | 33 |
| 600 | GGAGAAGCGACGCCCTTGAC | 32 |
| 601 | GGGAGAAGCGACGCCCTTGA | 31 |
| 602 | AGGGAGAAGCGACGCCCTTG | 30 |
| 603 | TAGGGAGAAGCGACGCCCTT | 29 |
| 604 | TTAGGGAGAAGCGACGCCCT | 28 |
| 605 | ATTAGGGAGAAGCGACGCCC | 27 |
| 606 | CATTAGGGAGAAGCGACGCC | 26 |
| 607 | CCCCCGATGCTCTTCACCTCGGG | 261 |
| 608 | CCCCGATGCTCTTCACCTCG | 262 |
| 609 | CCCGATGCTCTTCACCTCGG | 263 |
| 610 | CCGATGCTCTTCACCTCGGG | 264 |
| 611 | CGATGCTCTTCACCTCGGGA | 265 |
| 612 | GATGCTCTTCACCTCGGGAG | 266 |
| 613 | ATGCTCTTCACCTCGGGAGC | 267 |
| 614 | TGCTCTTCACCTCGGGAGCC | 268 |
| 615 | GCTCTTCACCTCGGGAGCCC | 269 |
| 616 | CTCTTCACCTCGGGAGCCCG | 270 |
| 617 | TCTTCACCTCGGGAGCCCGG | 271 |
| 618 | CTTCACCTCGGGAGCCCGGA | 272 |
| 619 | TTCACCTCGGGAGCCCGGAG | 273 |
| 620 | TCACCTCGGGAGCCCGGAGC | 274 |
| 621 | CACCTCGGGAGCCCGGAGCC | 275 |
| 622 | ACCTCGGGAGCCCGGAGCCC | 276 |
| 623 | CCTCGGGAGCCCGGAGCCCG | 277 |
| 624 | CTCGGGAGCCCGGAGCCCGT | 278 |
| 625 | TCGGGAGCCCGGAGCCCGTC | 279 |
| 626 | CGGGAGCCCGGAGCCCGTCA | 280 |
| 627 | GGGAGCCCGGAGCCCGTCAC | 281 |
| 628 | GGAGCCCGGAGCCCGTCACC | 282 |
| 629 | GAGCCCGGAGCCCGTCACCC | 283 |
| 630 | AGCCCGGAGCCCGTCACCCA | 284 |
| 631 | GCCCGGAGCCCGTCACCCAA | 285 |
| 632 | CCCGGAGCCCGTCACCCAAG | 286 |
| 633 | CCGGAGCCCGTCACCCAAGT | 287 |
| 634 | CGGAGCCCGTCACCCAAGTC | 288 |
| 635 | GGAGCCCGTCACCCAAGTCC | 289 |
| 636 | GAGCCCGTCACCCAAGTCCG | 290 |
| 637 | AGCCCGTCACCCAAGTCCGG | 291 |
| 638 | GCCCGTCACCCAAGTCCGGT | 292 |
| 639 | CCCGTCACCCAAGTCCGGTC | 293 |
| 640 | CCGTCACCCAAGTCCGGTCT | 294 |
| 641 | CGTCACCCAAGTCCGGTCTA | 295 |
| 642 | GTCACCCAAGTCCGGTCTAC | 296 |
| 643 | TCACCCAAGTCCGGTCTACC | 297 |
| 644 | CACCCAAGTCCGGTCTACCG | 298 |
| 645 | ACCCAAGTCCGGTCTACCGC | 299 |
| 646 | CCCAAGTCCGGTCTACCGCG | 300 |
| 647 | CCAAGTCCGGTCTACCGCGG | 301 |
| 648 | CAAGTCCGGTCTACCGCGGA | 302 |
| 649 | AAGTCCGGTCTACCGCGGAG | 303 |
| 650 | AGTCCGGTCTACCGCGGAGG | 304 |
| 651 | GTCCGGTCTACCGCGGAGGC | 305 |
| 652 | TCCGGTCTACCGCGGAGGCA | 306 |
| 653 | CCGGTCTACCGCGGAGGCAC | 307 |
| 654 | CGGTCTACCGCGGAGGCACT | 308 |
| 655 | GGTCTACCGCGGAGGCACTG | 309 |
| 656 | GTCTACCGCGGAGGCACTGT | 310 |
| 657 | TCTACCGCGGAGGCACTGTG | 311 |
| 658 | CTACCGCGGAGGCACTGTGG | 312 |
| 659 | TACCGCGGAGGCACTGTGGC | 313 |
| 660 | ACCGCGGAGGCACTGTGGCC | 314 |
| 661 | CCGCGGAGGCACTGTGGCCT | 315 |
| 662 | CGCGGAGGCACTGTGGCCTC | 316 |
| 663 | GCGGAGGCACTGTGGCCTCG | 317 |
| 664 | CGGAGGCACTGTGGCCTCGG | 318 |
| 665 | GGAGGCACTGTGGCCTCGGG | 319 |
| 666 | GAGGCACTGTGGCCTCGGGT | 320 |
| 667 | AGGCACTGTGGCCTCGGGTC | 321 |
| 668 | GGCACTGTGGCCTCGGGTCG | 322 |
| 669 | GCACTGTGGCCTCGGGTCGG | 323 |
| 670 | CACTGTGGCCTCGGGTCGGC | 324 |
| 671 | ACTGTGGCCTCGGGTCGGCC | 325 |
| 672 | CTGTGGCCTCGGGTCGGCCC | 326 |
| 673 | TGTGGCCTCGGGTCGGCCCC | 327 |
| 674 | GTGGCCTCGGGTCGGCCCCG | 328 |
| 675 | TGGCCTCGGGTCGGCCCCGG | 329 |
| 676 | GGCCTCGGGTCGGCCCCGGA | 330 |
| 677 | GCCTCGGGTCGGCCCCGGAG | 331 |
| 678 | CCTCGGGTCGGCCCCGGAGC | 332 |
| 679 | CTCGGGTCGGCCCCGGAGCG | 333 |
| 680 | TCGGGTCGGCCCCGGAGCGA | 334 |
| 681 | CGGGTCGGCCCCGGAGCGAG | 335 |
| 682 | GGGTCGGCCCCGGAGCGAGG | 336 |
| 683 | GGTCGGCCCCGGAGCGAGGC | 337 |
| 684 | GTCGGCCCCGGAGCGAGGCC | 338 |
| 685 | TCGGCCCCGGAGCGAGGCCT | 339 |
| 686 | CGGCCCCGGAGCGAGGCCTC | 340 |
| 687 | GGCCCCGGAGCGAGGCCTCC | 341 |
| 688 | GCCCCGGAGCGAGGCCTCCA | 342 |
| 689 | CCCCGGAGCGAGGCCTCCAG | 343 |
| 690 | CCCGGAGCGAGGCCTCCAGA | 344 |
| 691 | CCGGAGCGAGGCCTCCAGAA | 345 |
| 692 | CGGAGCGAGGCCTCCAGAAC | 346 |
| 693 | GGAGCGAGGCCTCCAGAACT | 347 |
| 694 | GAGCGAGGCCTCCAGAACTA | 348 |
| 695 | AGCGAGGCCTCCAGAACTAC | 349 |
| 696 | GCGAGGCCTCCAGAACTACC | 350 |
| 697 | CGAGGCCTCCAGAACTACCA | 351 |
| 698 | GCCCCCGATGCTCTTCACCT | 260 |
| 699 | AGCCCCCGATGCTCTTCACC | 259 |
| 700 | CAGCCCCCGATGCTCTTCAC | 258 |
| 701 | TCAGCCCCCGATGCTCTTCA | 257 |
| 702 | CTCAGCCCCCGATGCTCTTC | 256 |
| 703 | CCTCAGCCCCCGATGCTCTT | 255 |
| 704 | ACCTCAGCCCCCGATGCTCT | 254 |
| 705 | CACCTCAGCCCCCGATGCTC | 253 |
| 706 | CCACCTCAGCCCCCGATGCT | 252 |
| 707 | CCCACCTCAGCCCCCGATGC | 251 |
| 708 | TCCCACCTCAGCCCCCGATG | 250 |
| 709 | GTCCCACCTCAGCCCCCGAT | 249 |
| 710 | GGTCCCACCTCAGCCCCCGA | 248 |
| 711 | AGGTCCCACCTCAGCCCCCG | 247 |
| 712 | CGGGTCGGCCCCGGAGCGAGGCC | 335 |
| 713 | GGGTCGGCCCCGGAGCGAGG | 336 |
| 714 | GGTCGGCCCCGGAGCGAGGC | 337 |
| 715 | GTCGGCCCCGGAGCGAGGCC | 338 |
| 716 | TCGGCCCCGGAGCGAGGCCT | 339 |
| 717 | CGGCCCCGGAGCGAGGCCTC | 340 |
| 718 | GGCCCCGGAGCGAGGCCTCC | 341 |
| 719 | GCCCCGGAGCGAGGCCTCCA | 342 |
| 720 | CCCCGGAGCGAGGCCTCCAG | 343 |
| 721 | CCCGGAGCGAGGCCTCCAGA | 344 |
| 722 | CCGGAGCGAGGCCTCCAGAA | 345 |
| 723 | CGGAGCGAGGCCTCCAGAAC | 346 |
| 724 | GGAGCGAGGCCTCCAGAACT | 347 |
| 725 | GAGCGAGGCCTCCAGAACTA | 348 |
| 726 | AGCGAGGCCTCCAGAACTAC | 349 |
| 727 | GCGAGGCCTCCAGAACTACC | 350 |
| 728 | CGAGGCCTCCAGAACTACCA | 351 |
| 729 | TCGGGTCGGCCCCGGAGCGA | 334 |
| 730 | CTCGGGTCGGCCCCGGAGCG | 333 |
| 731 | CCTCGGGTCGGCCCCGGAGC | 332 |
| 732 | GCCTCGGGTCGGCCCCGGAG | 331 |
| 733 | GGCCTCGGGTCGGCCCCGGA | 330 |
| 734 | TGGCCTCGGGTCGGCCCCGG | 329 |
| 735 | GTGGCCTCGGGTCGGCCCCG | 328 |
| 736 | TGTGGCCTCGGGTCGGCCCC | 327 |
| 737 | CTGTGGCCTCGGGTCGGCCC | 326 |
| 738 | ACTGTGGCCTCGGGTCGGCC | 325 |
| 739 | CACTGTGGCCTCGGGTCGGC | 324 |
| 740 | GCACTGTGGCCTCGGGTCGG | 323 |
| 741 | GGCACTGTGGCCTCGGGTCG | 322 |
| 742 | AGGCACTGTGGCCTCGGGTC | 321 |
| 743 | GAGGCACTGTGGCCTCGGGT | 320 |
| 744 | GGAGGCACTGTGGCCTCGGG | 319 |
| 745 | CGGAGGCACTGTGGCCTCGG | 318 |
| 746 | GCGGAGGCACTGTGGCCTCG | 317 |
| 747 | CGCGGAGGCACTGTGGCCTC | 316 |
| 748 | CCGCGGAGGCACTGTGGCCT | 315 |
| 749 | ACCGCGGAGGCACTGTGGCC | 314 |
| 750 | TACCGCGGAGGCACTGTGGC | 313 |
| 751 | CTACCGCGGAGGCACTGTGG | 312 |
| 752 | TCTACCGCGGAGGCACTGTG | 311 |
| 753 | GTCTACCGCGGAGGCACTGT | 310 |
| 754 | GGTCTACCGCGGAGGCACTG | 309 |
| 755 | CGGTCTACCGCGGAGGCACT | 308 |
| 756 | CCGGTCTACCGCGGAGGCAC | 307 |
| 757 | TCCGGTCTACCGCGGAGGCA | 306 |
| 758 | GTCCGGTCTACCGCGGAGGC | 305 |
| 759 | AGTCCGGTCTACCGCGGAGG | 304 |
| 760 | AAGTCCGGTCTACCGCGGAG | 303 |
| 761 | CAAGTCCGGTCTACCGCGGA | 302 |
| 762 | CCAAGTCCGGTCTACCGCGG | 301 |
| 763 | CCCAAGTCCGGTCTACCGCG | 300 |
| 764 | ACCCAAGTCCGGTCTACCGC | 299 |
| 765 | CACCCAAGTCCGGTCTACCG | 298 |
| 766 | TCACCCAAGTCCGGTCTACC | 297 |
| 767 | GTCACCCAAGTCCGGTCTAC | 296 |
| 768 | CGTCACCCAAGTCCGGTCTA | 295 |
| 769 | CCGTCACCCAAGTCCGGTCT | 294 |
| 770 | CCCGTCACCCAAGTCCGGTC | 293 |
| 771 | GCCCGTCACCCAAGTCCGGT | 292 |
| 772 | AGCCCGTCACCCAAGTCCGG | 291 |
| 773 | GAGCCCGTCACCCAAGTCCG | 290 |
| 774 | GGAGCCCGTCACCCAAGTCC | 289 |
| 775 | CGGAGCCCGTCACCCAAGTC | 288 |
| 776 | CCGGAGCCCGTCACCCAAGT | 287 |
| 777 | CCCGGAGCCCGTCACCCAAG | 286 |
| 778 | GCCCGGAGCCCGTCACCCAA | 285 |
| 779 | AGCCCGGAGCCCGTCACCCA | 284 |
| 780 | GAGCCCGGAGCCCGTCACCC | 283 |
| 781 | GGAGCCCGGAGCCCGTCACC | 282 |
| 782 | GGGAGCCCGGAGCCCGTCAC | 281 |
| 783 | CGGGAGCCCGGAGCCCGTCA | 280 |
| 784 | TCGGGAGCCCGGAGCCCGTC | 279 |
| 785 | CTCGGGAGCCCGGAGCCCGT | 278 |
| 786 | CCTCGGGAGCCCGGAGCCCG | 277 |
| 787 | ACCTCGGGAGCCCGGAGCCC | 276 |
| 788 | CACCTCGGGAGCCCGGAGCC | 275 |
| 789 | TCACCTCGGGAGCCCGGAGC | 274 |
| 790 | TTCACCTCGGGAGCCCGGAG | 273 |
| 791 | CTTCACCTCGGGAGCCCGGA | 272 |
| 792 | TCTTCACCTCGGGAGCCCGG | 271 |
| 793 | CTCTTCACCTCGGGAGCCCG | 270 |
| 794 | GCTCTTCACCTCGGGAGCCC | 269 |
| 795 | TGCTCTTCACCTCGGGAGCC | 268 |
| 796 | ATGCTCTTCACCTCGGGAGC | 267 |
| 797 | GATGCTCTTCACCTCGGGAG | 266 |
| 798 | CGATGCTCTTCACCTCGGGA | 265 |
| 799 | CCGATGCTCTTCACCTCGGG | 264 |
| 800 | CCCGATGCTCTTCACCTCGG | 263 |
| 801 | CCCCGATGCTCTTCACCTCG | 262 |
| 802 | CCCCCGATGCTCTTCACCTC | 261 |
| 803 | GCCCCCGATGCTCTTCACCT | 260 |
| 804 | AGCCCCCGATGCTCTTCACC | 259 |
| 805 | CAGCCCCCGATGCTCTTCAC | 258 |
| 806 | TCAGCCCCCGATGCTCTTCA | 257 |
| 807 | CTCAGCCCCCGATGCTCTTC | 256 |
| 808 | CCTCAGCCCCCGATGCTCTT | 255 |
| 809 | ACCTCAGCCCCCGATGCTCT | 254 |
| 810 | CACCTCAGCCCCCGATGCTC | 253 |
| 811 | CCACCTCAGCCCCCGATGCT | 252 |
| 812 | CCCACCTCAGCCCCCGATGC | 251 |
| 813 | TCCCACCTCAGCCCCCGATG | 250 |
| 814 | GTCCCACCTCAGCCCCCGAT | 249 |
| 815 | GGTCCCACCTCAGCCCCCGA | 248 |
| 816 | AGGTCCCACCTCAGCCCCCG | 247 |
| 817 | GCCCGGCAGCGGCCCCCAGAGGCCG | 475 |
| 818 | CCCGGCAGCGGCCCCCAGAG | 476 |
| 819 | CCGGCAGCGGCCCCCAGAGG | 477 |
| 820 | CGGCAGCGGCCCCCAGAGGC | 478 |
| 821 | GGCAGCGGCCCCCAGAGGCC | 479 |
| 822 | GCAGCGGCCCCCAGAGGCCG | 480 |
| 823 | CAGCGGCCCCCAGAGGCCGG | 481 |
| 824 | AGCGGCCCCCAGAGGCCGGG | 482 |
| 825 | GCGGCCCCCAGAGGCCGGGC | 483 |
| 826 | CGGCCCCCAGAGGCCGGGCT | 484 |
| 827 | GGCCCCCAGAGGCCGGGCTG | 485 |
| 828 | GCCCCCAGAGGCCGGGCTGG | 486 |
| 829 | CCCCCAGAGGCCGGGCTGGG | 487 |
| 830 | CCCCAGAGGCCGGGCTGGGA | 488 |
| 831 | CCCAGAGGCCGGGCTGGGAA | 489 |
| 832 | GGCCCGGCAGCGGCCCCCAG | 474 |
| 833 | AGGCCCGGCAGCGGCCCCCA | 473 |
| 834 | CAGGCCCGGCAGCGGCCCCC | 472 |
| 835 | ACAGGCCCGGCAGCGGCCCC | 471 |
| 836 | CACAGGCCCGGCAGCGGCCC | 470 |
| 837 | TCACAGGCCCGGCAGCGGCC | 469 |
| 838 | CTCACAGGCCCGGCAGCGGC | 468 |
| 839 | GCTCACAGGCCCGGCAGCGG | 467 |
| 840 | GGCTCACAGGCCCGGCAGCG | 466 |
| 841 | AGGCTCACAGGCCCGGCAGC | 465 |
| 842 | CAGGCTCACAGGCCCGGCAG | 464 |
| 843 | ACAGGCTCACAGGCCCGGCA | 463 |
| 844 | CACAGGCTCACAGGCCCGGC | 462 |
| 845 | CCACAGGCTCACAGGCCCGG | 461 |
| 846 | TCCACAGGCTCACAGGCCCG | 460 |
| 847 | CGGTCTACCGCGGAGGCACTGTGGCCTCGG | 308 |
| 848 | GGTCTACCGCGGAGGCACTG | 309 |
| 849 | GTCTACCGCGGAGGCACTGT | 310 |
| 850 | TCTACCGCGGAGGCACTGTG | 311 |
| 851 | CTACCGCGGAGGCACTGTGG | 312 |
| 852 | TACCGCGGAGGCACTGTGGC | 313 |
| 853 | ACCGCGGAGGCACTGTGGCC | 314 |
| 854 | CCGCGGAGGCACTGTGGCCT | 315 |
| 855 | CGCGGAGGCACTGTGGCCTC | 316 |
| 856 | GCGGAGGCACTGTGGCCTCG | 317 |
| 857 | CGGAGGCACTGTGGCCTCGG | 318 |
| 858 | GGAGGCACTGTGGCCTCGGG | 319 |
| 859 | GAGGCACTGTGGCCTCGGGT | 320 |
| 860 | AGGCACTGTGGCCTCGGGTC | 321 |
| 861 | GGCACTGTGGCCTCGGGTCG | 322 |
| 862 | GCACTGTGGCCTCGGGTCGG | 323 |
| 863 | CACTGTGGCCTCGGGTCGGC | 324 |
| 864 | ACTGTGGCCTCGGGTCGGCC | 325 |
| 865 | CTGTGGCCTCGGGTCGGCCC | 326 |
| 866 | TGTGGCCTCGGGTCGGCCCC | 327 |
| 867 | GTGGCCTCGGGTCGGCCCCG | 328 |
| 868 | TGGCCTCGGGTCGGCCCCGG | 329 |
| 869 | GGCCTCGGGTCGGCCCCGGA | 330 |
| 870 | GCCTCGGGTCGGCCCCGGAG | 331 |
| 871 | CCTCGGGTCGGCCCCGGAGC | 332 |
| 872 | CTCGGGTCGGCCCCGGAGCG | 333 |
| 873 | TCGGGTCGGCCCCGGAGCGA | 334 |
| 874 | CGGGTCGGCCCCGGAGCGAG | 335 |
| 875 | GGGTCGGCCCCGGAGCGAGG | 336 |
| 876 | GGTCGGCCCCGGAGCGAGGC | 337 |
| 877 | GTCGGCCCCGGAGCGAGGCC | 338 |
| 878 | TCGGCCCCGGAGCGAGGCCT | 339 |
| 879 | CGGCCCCGGAGCGAGGCCTC | 340 |
| 880 | GGCCCCGGAGCGAGGCCTCC | 341 |
| 881 | GCCCCGGAGCGAGGCCTCCA | 342 |
| 882 | CCCCGGAGCGAGGCCTCCAG | 343 |
| 883 | CCCGGAGCGAGGCCTCCAGA | 344 |
| 884 | CCGGAGCGAGGCCTCCAGAA | 345 |
| 885 | CGGAGCGAGGCCTCCAGAAC | 346 |
| 886 | GGAGCGAGGCCTCCAGAACT | 347 |
| 887 | GAGCGAGGCCTCCAGAACTA | 348 |
| 888 | AGCGAGGCCTCCAGAACTAC | 349 |
| 889 | GCGAGGCCTCCAGAACTACC | 350 |
| 890 | CGAGGCCTCCAGAACTACCA | 351 |
| 891 | CCGGTCTACCGCGGAGGCAC | 307 |
| 892 | TCCGGTCTACCGCGGAGGCA | 306 |
| 893 | GTCCGGTCTACCGCGGAGGC | 305 |
| 894 | AGTCCGGTCTACCGCGGAGG | 304 |
| 895 | AAGTCCGGTCTACCGCGGAG | 303 |
| 896 | CAAGTCCGGTCTACCGCGGA | 302 |
| 897 | CCAAGTCCGGTCTACCGCGG | 301 |
| 898 | CCCAAGTCCGGTCTACCGCG | 300 |
| 899 | ACCCAAGTCCGGTCTACCGC | 299 |
| 900 | CACCCAAGTCCGGTCTACCG | 298 |
| 901 | TCACCCAAGTCCGGTCTACC | 297 |
| 902 | GTCACCCAAGTCCGGTCTAC | 296 |
| 903 | CGTCACCCAAGTCCGGTCTA | 295 |
| 904 | CCGTCACCCAAGTCCGGTCT | 294 |
| 905 | CCCGTCACCCAAGTCCGGTC | 293 |
| 906 | GCCCGTCACCCAAGTCCGGT | 292 |
| 907 | AGCCCGTCACCCAAGTCCGG | 291 |
| 908 | GAGCCCGTCACCCAAGTCCG | 290 |
| 909 | GGAGCCCGTCACCCAAGTCC | 289 |
| 910 | CGGAGCCCGTCACCCAAGTC | 288 |
| 911 | CCGGAGCCCGTCACCCAAGT | 287 |
| 912 | CCCGGAGCCCGTCACCCAAG | 286 |
| 913 | GCCCGGAGCCCGTCACCCAA | 285 |
| 914 | AGCCCGGAGCCCGTCACCCA | 284 |
| 915 | GAGCCCGGAGCCCGTCACCC | 283 |
| 916 | GGAGCCCGGAGCCCGTCACC | 282 |
| 917 | GGGAGCCCGGAGCCCGTCAC | 281 |
| 918 | CGGGAGCCCGGAGCCCGTCA | 280 |
| 919 | TCGGGAGCCCGGAGCCCGTC | 279 |
| 920 | CTCGGGAGCCCGGAGCCCGT | 278 |
| 921 | CCTCGGGAGCCCGGAGCCCG | 277 |
| 922 | ACCTCGGGAGCCCGGAGCCC | 276 |
| 923 | CACCTCGGGAGCCCGGAGCC | 275 |
| 924 | TCACCTCGGGAGCCCGGAGC | 274 |
| 925 | TTCACCTCGGGAGCCCGGAG | 273 |
| 926 | CTTCACCTCGGGAGCCCGGA | 272 |
| 927 | TCTTCACCTCGGGAGCCCGG | 271 |
| 928 | CTCTTCACCTCGGGAGCCCG | 270 |
| 929 | GCTCTTCACCTCGGGAGCCC | 269 |
| 930 | TGCTCTTCACCTCGGGAGCC | 268 |
| 931 | ATGCTCTTCACCTCGGGAGC | 267 |
| 932 | GATGCTCTTCACCTCGGGAG | 266 |
| 933 | CGATGCTCTTCACCTCGGGA | 265 |
| 934 | CCGATGCTCTTCACCTCGGG | 264 |
| 935 | CCCGATGCTCTTCACCTCGG | 263 |
| 936 | CCCCGATGCTCTTCACCTCG | 262 |
| 937 | CCCCCGATGCTCTTCACCTC | 261 |
| 938 | GCCCCCGATGCTCTTCACCT | 260 |
| 939 | AGCCCCCGATGCTCTTCACC | 259 |
| 940 | CAGCCCCCGATGCTCTTCAC | 258 |
| 941 | TCAGCCCCCGATGCTCTTCA | 257 |
| 942 | CTCAGCCCCCGATGCTCTTC | 256 |
| 943 | CCTCAGCCCCCGATGCTCTT | 255 |
| 944 | ACCTCAGCCCCCGATGCTCT | 254 |
| 945 | CACCTCAGCCCCCGATGCTC | 253 |
| 946 | CCACCTCAGCCCCCGATGCT | 252 |
| 947 | CCCACCTCAGCCCCCGATGC | 251 |
| 948 | TCCCACCTCAGCCCCCGATG | 250 |
| 949 | GTCCCACCTCAGCCCCCGAT | 249 |
| 950 | GGTCCCACCTCAGCCCCCGA | 248 |
| 951 | AGGTCCCACCTCAGCCCCCG | 247 |
| 952 | ACAAAAACTAGCCGGGCGTGGTGGGGCACGCC | 735 |
| 953 | CAAAAACTAGCCGGGCGTGG | 736 |
| 954 | AAAAACTAGCCGGGCGTGGT | 737 |
| 955 | AAAACTAGCCGGGCGTGGTG | 738 |
| 956 | AAACTAGCCGGGCGTGGTGG | 739 |
| 957 | AACTAGCCGGGCGTGGTGGG | 740 |
| 958 | ACTAGCCGGGCGTGGTGGGG | 741 |
| 959 | CTAGCCGGGCGTGGTGGGGC | 742 |
| 960 | TAGCCGGGCGTGGTGGGGCA | 743 |
| 961 | AGCCGGGCGTGGTGGGGCAC | 744 |
| 962 | GCCGGGCGTGGTGGGGCACG | 745 |
| 963 | CCGGGCGTGGTGGGGCACGC | 746 |
| 964 | CGGGCGTGGTGGGGCACGCC | 747 |
| 965 | GGGCGTGGTGGGGCACGCCT | 748 |
| 966 | GGCGTGGTGGGGCACGCCTA | 749 |
| 967 | GCGTGGTGGGGCACGCCTAT | 750 |
| 968 | CGTGGTGGGGCACGCCTATA | 751 |
| 969 | GTGGTGGGGCACGCCTATAA | 752 |
| 970 | TGGTGGGGCACGCCTATAAT | 753 |
| 971 | GGTGGGGCACGCCTATAATC | 754 |
| 972 | GTGGGGCACGCCTATAATCC | 755 |
| 973 | TGGGGCACGCCTATAATCCC | 756 |
| 974 | GGGGCACGCCTATAATCCCA | 757 |
| 975 | GGGCACGCCTATAATCCCAG | 758 |
| 976 | GGCACGCCTATAATCCCAGC | 759 |
| 977 | GCACGCCTATAATCCCAGCT | 760 |
| 978 | CACGCCTATAATCCCAGCTT | 761 |
| 979 | ACGCCTATAATCCCAGCTTA | 762 |
| 980 | CGCCTATAATCCCAGCTTAA | 763 |
| 981 | TACAAAAACTAGCCGGGCGT | 734 |
| 982 | ATACAAAAACTAGCCGGGCG | 733 |
| 983 | AATACAAAAACTAGCCGGGC | 732 |

Hot Zones (Relative upstream location to gene start site)
1-1200
1850-2200
2550-3000
3300-3500

### Examples

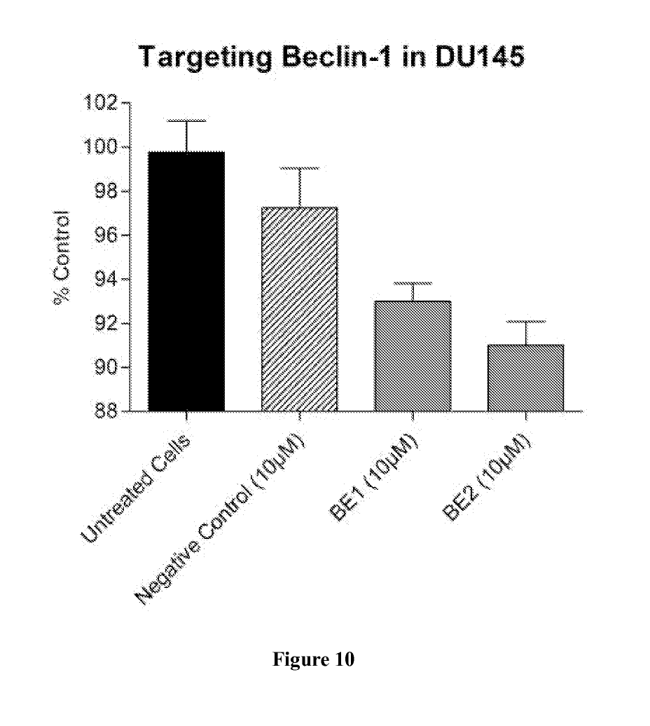

FIG. 10 shows that BE1 (11) and BE2 (12), both at 10 μM, demonstrated statistically significant (P<0.05) inhibition compared to the untreated control inhibition values in DU145 (human prostate cell line). The negative control did not produce a statistically significant difference compared to the untreated control. The Beclin-1 sequences BE1 (11) and BE2 (12) fit the independent and dependent DNAi motif claims.

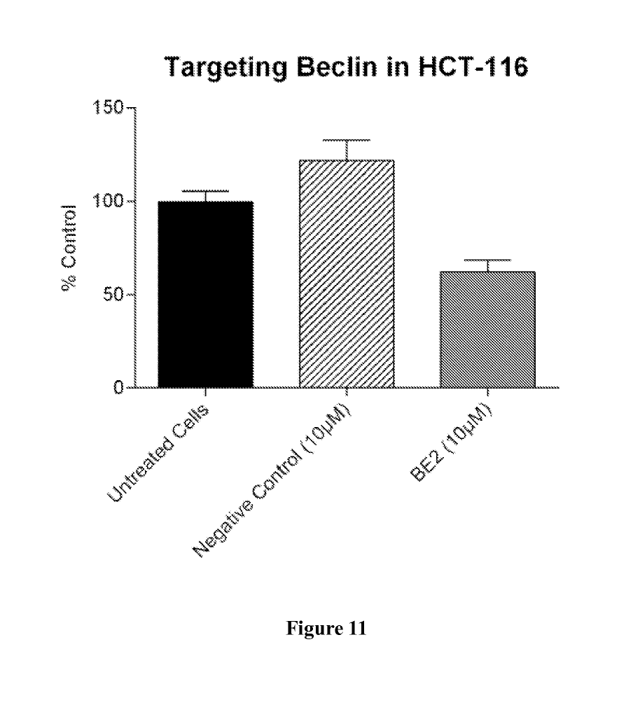

FIG. 11 shows that BE2 (12) at 10 μM demonstrated statistically significant (P<0.05) inhibition compared to the untreated and negative control values in HCT-116 (human colorectal carcinoma). The negative control did not produce a statistically significant difference compared to the untreated control. BE2 (12) fit the independent and dependent DNAi motif claims.

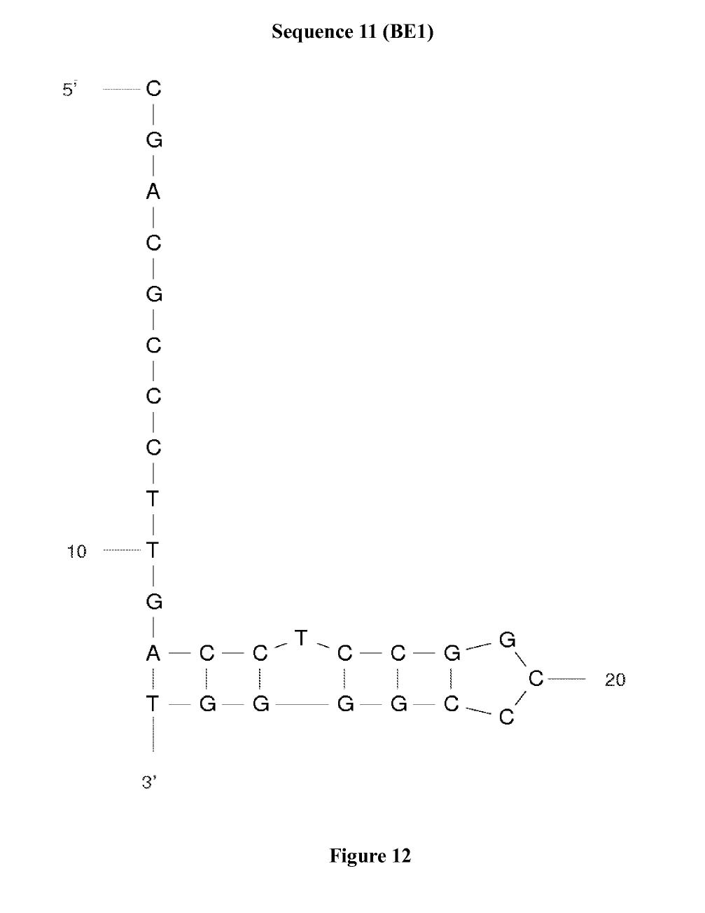

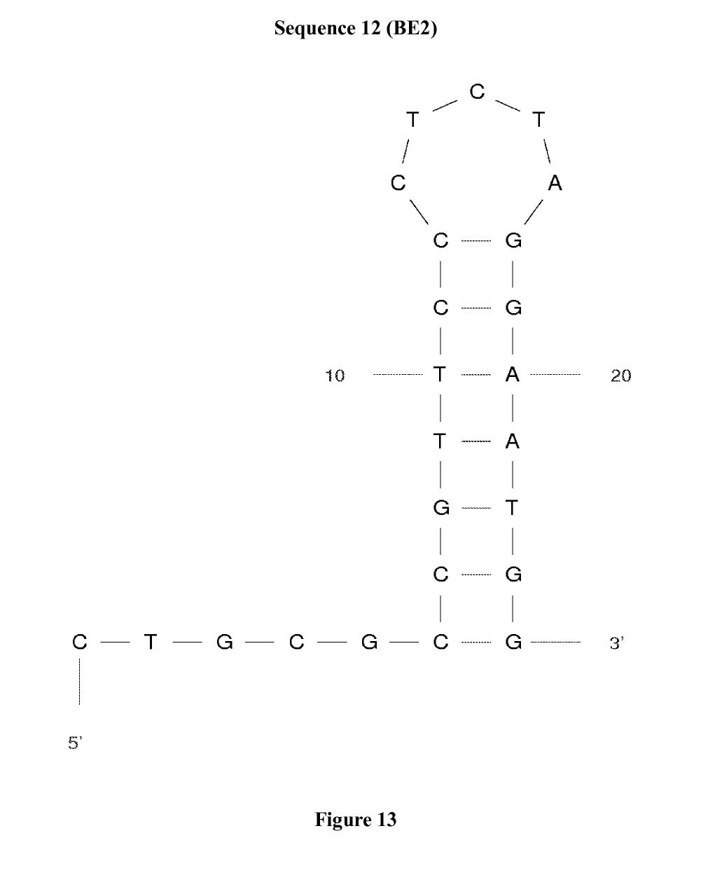

The secondary structures for BE1 and BE2 are shown in FIGS. 12 and 13. Sequence 11 (BE1) is shown in FIG. 12 and Sequence 12 (BE2) is shown in FIG. 13.

Genetic Code (5′ Upstream Region)
(SEQ ID NO: 11951)
ACTTACCACCCTCAGTGGTTTCCAGATAACATAGGCCTTCCTGAATCCCC
CAGTTGAAGCAGCTCCTCCCACCCTGCCCCCACTTACTCTCTATCACATC
ACCTTCTTACCTACTGTATTAGCTTTCTAGGGCTGCTGTAGCAAAGTACC
ACAAAGTGGATGGCTTAGAACCAAAGAAATATATTGTCTCAGAGTTCTGG
ATGCCAGAAATCCAAAATTAAGGTGTCAGCAGGACCATGTTCCTTCTAAG
GGAGCCAGAGAAGTATCTGTTCCAGACCTCTTTCCTGGCTTTTGGTAGCC
TCAGGTCTTCCTTGGCTTACAGATCACCCTGTGTCTCTTTACATCATCTT
CCCTCAGACACGGTACATGTCTGTCTCTGTGTCCAGATTGCCCCTATTTA
TAAGGACGCAGTCATATTGGTCTAGGGCTAACATCAATGACCTCATCTGC
AACGATCCTATTTCCAAAAAAGGTCACATTCCCATGTGTTAGTCCCAGAT
GTTAGGACTTCAACATCTTTTGGGGGACATCATTCAACCCATAATATCTG
CCATTATCTGAAATTATCTTATTAACTTGGTTACATGTTTACTGTCAAAT
TCTCTCCTCTGGAATATAAACTATTAGAGCAGTTCACCAGTATATCCTCT
CAGACCTAGAATAGGGACTGGCACATAGTAGATGCTCAATAAACATCTGT
TGAATCGATGACTGAGGATATGTTGTGTATTATTCACAATCCCTCAAGCA
CTACATACACTGATTACATATACTTCCCAAGTGTGAGGATACACAGAGCA
TTCACTATGTAACAGTCATTCCCCTCCATTCCAAATGTATCAGCTCATTT
ATCACACTACCCTTTATGATATTTACTACTGTATACTATTAATCTCATTT
TGTAAATAAGAAAACAAAGCACAGAACAGTTGAATAAATTGCATAAGGTC
ACATGGTTAGTGGATGGTAAAGAACCAGGTGGTCTCAACTTCCAAATCCT
CAGTTGTAACACTATACCCCCTACCTCTCTAGAAGCCCGTTACTTCTCTA
TGCGTTTCTGAGATGTTAGGGACAGCCAAGCAGGAAGAAACGCAGGACTA
TGAAGCAGCCACACCAGGACTAGGTGAGAATTCTTTGGGGATGATTCCAG
TCACCTCCCCTAAAGGGGCTTTCATGCTGAAAGAGCCAAGAGGAAGAAGG
ATTGTAAACACTATCCCTAGTCACAAAACCGGGAGAAAAATCAATCTAGT
TCCACATATCACATCCAATACCAACTATAAGAAACCACATACATTTAAAA
GAAAAGAAAGACACTTCTGGAGGTGGGAATAACTTTCTAAGCAGTATAAG
TCATCAAGAAAAATAAGCAGATTTGACTTGAAAATTTAAAACTTCCTGAA
CATCTGGAAAATAATTAAAGCATTCATGAAAAATTACTAAAAATACTGAG
AAAAATACTAATAATCCAATACCTAAATAATCAAAGAATGCAAACATAAT
TCAGAAAAAAGTAACTACTGCTTGAGCCCGGGAGGCGGAGATTCCAGTGA
GCTGATATTGCACCACTGCACTCCAGCCTGGGTGACAGAGTGAGACCGTG
TCTCTTTTTTTTTTTTTTTTTAAAAAAAGGCCGGGCATGGTGGCTCACAC
CTGTAATCCCAGCACTTTGGGAGGCCAAGGCGGGCAGATCAGGAGGTCAG
GAGATTGAGACCATCCTGGCTAACATGGTGAAACCGTCTCCACTAAAAAT
ATAAAAAATTAGCCGGGTGTAGTGGTGGGCGCCTGTAGTCCCAGCTACTC
GGGAGGCTGAGACAGGAGAATGGCGTGAACCCAGGAGGCAGAGGTTGCAG
TGAGCCGAGATGGCACCACTGCACTCCAGCCTGGGCAACAGAGCAAGACT
CCATCTCAAAAAAAAAAAAAAAAGTAACTACAATAAGCAAATACATAGCA
AAAAGTTCAGCCTTACCAGCAATCAATGATGCTAATTAAAATAACAAGGA
AGTGCCATTTTTTGCTTTTGTTCCCCAAATATATGATACCCAATACTGGC
CAAGGCAATATGAAAACAGGCTTCCTCATACATTACTGGAAGCAGAATAT
AGTTATGTGCAAGCACTTTGGAAAATGATTCCCAGTGTTAAGGAAGAGAC
ATTAAATAGCTGACACACTCTTAATTCTGTAGTCCCAGTTATGAGTCTCT
ATCATAAGTAGCCAGCTCTTCATTGCAGGATTATTGTAATCACCCACAGG
GGAAATAGTAGAATTTCCAGCGGTAAAAAAATACACTAAGGCAGTACATT
TAGTGTAGTGTAATGTAGCCATGATAACTACAATAACTGTGTAGCAACAT
AGAAAAATGTTAAATTTAAAAAGCAGAAGCCTGGGCAACAAAGTGAGACC
CCATCTCTTTTTTTTTTTGAGATGGCGTCTCGCTCTGTCACCGAGGCTGG
AGTGCAGTGTGAGACCACATCTCTACAAAAAATTTTAAAAATTAGCTGGG
CATGGTAGTGATCACCTGTGGTCCCTGCTACACTGGAGGTTGAAGCAAGA
GGATTGCTTGAGCCAGGAAGTCAAATCTGCAGTGAGCCATGTTTGTTTGT
TCCGCTTCACTCCAGCCTGGGTAACAGAGTAAGACACTGTCTCAAAATAA
AAATAAAATAGACAATACTACATACAATTTTGGGTTAAGCAGTGGTTTCT
TTTACACCAAAAGCATAAACATTGGACTTTATTGAAATGAAAAACTTTTG
GCCAGGCACATTGGCTCACACCTGTAATCTCAGCACTTTGGGAGGCCACA
GTGGGGGATTGCAAGGGGAGATGGGAAATGTTCTAAAACTGGATTATGGT
GATAGTTGGGCAACTGTGTAAATTTACTAAAAATTATTGAACTGTACATT
TAAAAAGTGTGAGTCTTATGGTATGTAAATTATACCCCATAAAGTTGTTT
TTAAAAATGAAGTAAGTCCCTCTGCTCAAGACCCAGTCATCTCATCTCAT
TCAAAGTGAAAGCCAGAGCTTTACAATCCCTATAAGAGCCTAGGTGGTAG
CTCAACACTCTTACCTCCCTCACCCCATTTTCTGTATCTCTTTTCGTTGC
CCATCTTCTAGCCACACCAGCCTCTGCTAATCCCCAAACAGGTACCCTCT
GTGCTCTTGCTGTTCCCTTGGCCTAGAATGCTCTTCCTTAAGATGCAGGT
AAGAATTCCTTCCTCACCTTCTTCAAGCTTTTATTTGAATATCACTTTCT
TTTTTTGTTGGTTTTGTGTGTGTGTGTGGGGGGGGGGGGTTTGAGATGGA
GTTTCCTTCTGTCGCCCAGGCTGGAGTGCAGTGGCATGATCTCGACTCAC
TGCAACCTCCGCCTCCGGGGGTCAAGCGATTTTCCTACCCCAGCCTCCTG
AGTAGCTGGGATTACAGGCGCACGCCACCATGCCCAGCTAATTGTATTTT
TTAGTAGAGACGGGATTTAACCATTTTGGCCAGGCTGGTCTCGAACTCCT
GACCTTGTGATCCGCCCGCCTCGGCCTCCCAAAGTGCTGGAATTACAAGC
GTGAGCCACCATGCCCGGCCTTTTGTTGTTGCTGTTGTTGTTCTGAGATG
GAGCCTTGCCCTGTCGCCCAGGCTGGAGTGCAGTGGCCCGATCTCGGCTC
ACTGCAACCTCCACCTCCCAGGTTCAAGCGATTCTCCTGCCTCAGCCTCC
CGAGTAGCTGGGATTAAGCTGGGATTATAGGCGTGCCCCACCACGCCCGG
CTAGTTTTTGTATTTTTAGTAGAGACGGGGTTTCACTGTGTTGGCCAGGC
TGGTCTCGAACTCCTGACCTCACGTGATCCGCCCTCCTCGGCCTCCCCAA
GTGCTGAGATTACAGGCGTGAGCCACCGCGCCCGCCGCCCCCTGAATTTA
GAGAATAGCGGAGCCTCCCCATTCTCTGCGGCCTTGGCTCCTACACTTCC
CGTGGTAACCTTGTTCATCCGCTGAAGCCCGCTGCTTTTCCCAGCCCGGC
CTCTGGGGGCCGCTGCCGGGCCTGTGAGCCTGTGGACCAGGAGCTCCTGC
TGCCGTCGTAGCGTCACGTCCGGTCTCGGCGGAAGTTTTCCGGCGGCTAC
CGGGAAGTCGCTGAAGACAGAGCGATGGTAGTTCTGGAGGCCTCGCTCCG
GGGCCGACCCGAGGCCACAGTGCCTCCGCGGTAGACCGGACTTGGGTGAC
GGGCTCCGGGCTCCCGAGGTGAAGAGCATCGGGGGCTGAGGTGGGACCTT
AGAAGGGAGTCTGGGAACCCTCACGGCTCTTATTGGAGTCCCTTCCCTGA
CCCTGGGCTCTAAACTGCCTTTGCTCAGGCTGTCCCGGAAGCAGGTCCTC
CCCGTATCATACCATTCCTAGAGGGAACGGCGCAGGTTGGGACTTCCCTC
CCTTTACCATCGTCACCAAGGCATGTGGTAACCCCGGGCCGGAGGTCAAG
GGCGTCGCTTCTCCCTAATGTTGCCTCTTTTCCACGGCCTCAGGGATG

3) STAT3. Signal Transducers and Activators of Transcription 3 (STAT3) is a point of convergence for numerous oncogenic signalling pathways, is constitutively activated both in tumor cells and in immune cells in the tumor microenvironment. STAT3 inhibits the expression of mediators necessary for immune activation against tumor cells (Nature Reviews Immunology 7, 41-51; 2007; Proc Natl Acad Sci USA. 2006 Jul. 5; 103(27): 10151-10152) and promotes the production of immunosuppressive factors that further activate STAT3 in diverse immune-cell subsets, altering gene-expression. This restraining anti-tumor immune response and propagation of cross-talk between tumor cells and their immunological microenvironment leads to tumor-induced immunosuppression and enhanced tumor growth. STAT3 belongs to a protein family of transcription factors first characterized for their role in cytokine signaling that contain a site for specific tyrosine phosphorylation, a modification that results in a conformational rearrangement causing it to accumulate in the cell nucleus, bound to enhancer elements of target genes (Nat. Rev. Mol. Cell. Biol. 2002; 3:651-662). STAT3 is a substrate for the catalytic activity of the tyrosine kinase oncoprotein v-Src (Science. 1995; 269:81-83) and that phosphorylated STAT3 accumulated in many human cancers, suggesting that activated STAT3 may act as an oncogene (Cell. 1999; 98:295-303). In a recent issue of PNAS, Kasprzycka et al. (Proc. Natl. Acad. Sci. USA. 2006; 103:9964-9969) provided evidence that activated STAT3 in a tumor cell contributes to both cell survival and impaired immune surveillance by conferring properties of a T lymphocyte regulatory phenotype on a T cell lymphoma. Further it is recognized that STAT3 is stimulated by classic growth-promoting signals, such as activated growth factor receptors as well as a remarkable degree of diversity for the molecular mechanisms at the basis of STAT3 action including some noncanonical mechanisms of tumor progression that apparently do not rely on tyrosine phosphorylation or binding of homodimers to DNA (Cancer Res. 2005; 65:939-947), possibly involving pathways in malignant cells not directly regulating gene expression.

Isis Pharmaceuticals is developing an antisense against STAT3. In preclinical studies, ISIS-STAT3Rx demonstrated antitumor activity in animal models of human cancer. ISIS-STAT3Rx was tested in a Phase 1 study in patients with solid tumors and lymphoma who have relapsed or were refractory to multiple chemotherapy regimens and in a Phase 2 study in focused patient populations with advanced cancers that have been linked to STAT3 and who have failed all other treatment options with clear responses in patients with advanced cancer who were refractory to prior chemotherapy treatment. STAT3 is implicated in a variety of cancers, including brain, lung, breast, bone, liver and multiple myeloma to promote tumor cell growth and prevents cell death.

Protein: STAT3 Gene: STAT3 (Homo sapiens, chromosome 17, 40465343-40540513 [NCBI Reference Sequence: NC_{—}000017.10]; start site location: 40540405; strand: negative)

Gene Identification

| | |
|---|---|
| GeneID | 6774 |
| HGNC | 11364 |
| HPRD | 00026 |
| MIM | 102582 |

Targeted Sequences

| | | | Relative upstream location |
|---|---|---|---|
| | | | to gene start site |
| Sequence | Design | | (upstream promoter of the |
| ID No: | ID | Sequence (5′-3′) | two promoters) |
| 984 | ST1 | GGCCGAGGCACGCCGTCATGCA | −18 |
| 985 | ST2 | CCGGCCCTTGGCACCACGTGGTGGCGA | 345 |
| 986 | | TTGTTCCCTCGGCTGCGACGTCG | −135 |
| 987 | | CAGTCTGCGCCGCCGCAGCTCCGG | −92 |
| 988 | | CAGTGCGTGTGCGGTACAGCCG | 45 |
| 989 | | TGTGCTGGCTGTTCCGACAGTTCGGT | 140 |
| 990 | | TAACTACGCTATCCCGTGCGGCC | 1998449 |
| 991 | | TCGCCCAGCCCCAGCCTGGCCGAGGC | −35 |

Hot Zones (Relative upstream location to gene start site)
−200-200
300-400
1998400-1998500

### Examples

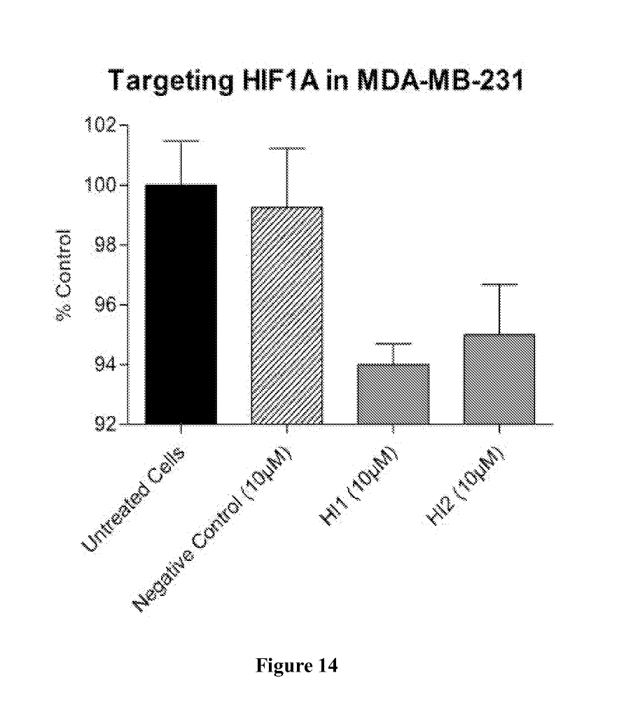

FIG. 14 shows ST1 (21) and ST2 (22), both at 10 μM, demonstrated statistically significant (P<0.05) inhibition compared to the untreated control inhibition values in MDA-MB-231 (human breast cell line). The negative control did not produce a statistically significant difference compared to the untreated control. The STAT3 sequence ST2 (22) fit the independent and dependent DNAi motif claims. The STAT3 sequence ST1 (21) is designed to the coding region of STAT3.

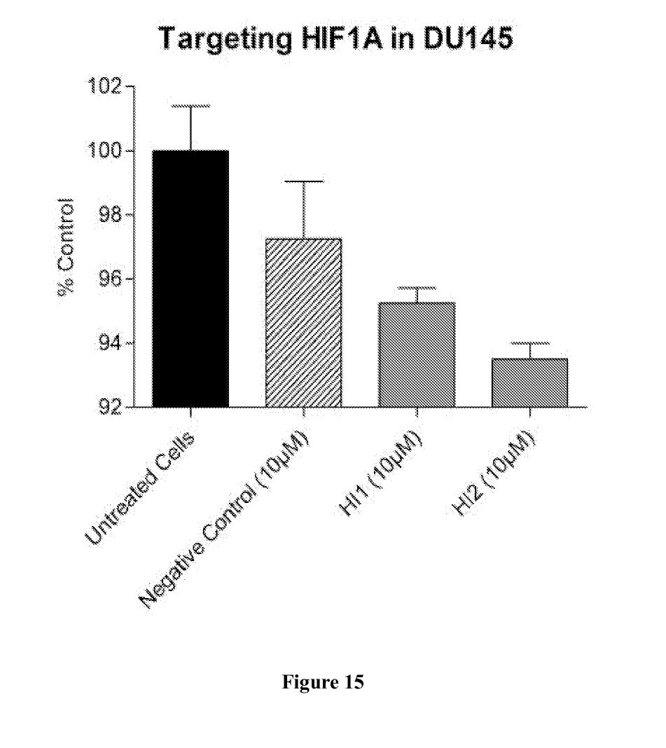

FIG. 15, which is similar to FIG. 12, shows ST1 (21) and ST2 (22), both at 10 μM, demonstrated statistically significant (P<0.05) inhibition compared to the untreated control inhibition values in DU145 (human prostate cell line). The negative control did not produce statistically significant difference compared to the untreated control. The STAT3 sequence ST2 (22) fit the independent and dependent DNAi motif claims. The STAT3 sequence ST1 (21) is designed to the coding region of STAT3.

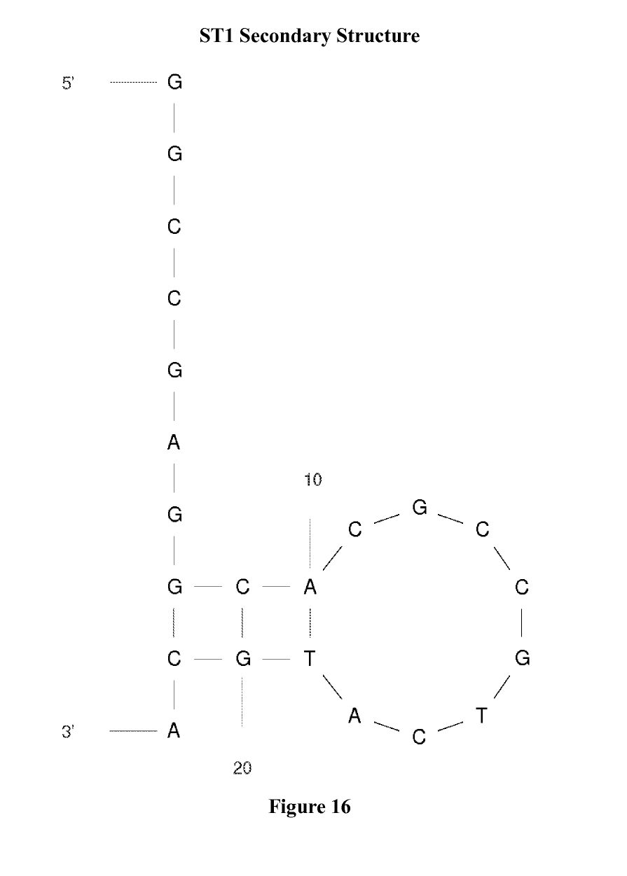

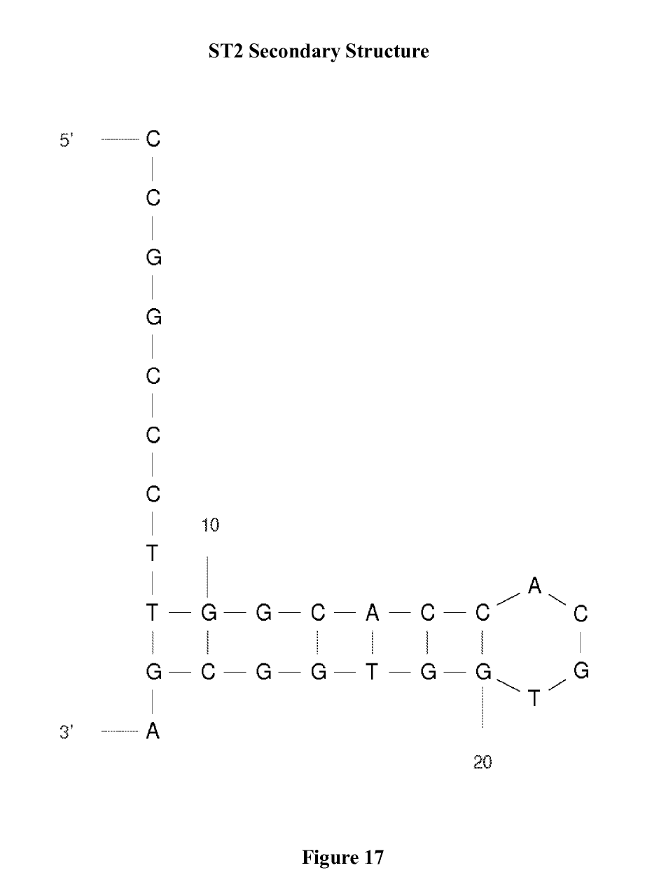

The secondary structures for ST1 and ST2 are shown in FIGS. 16 and 17. Sequence 21 (ST1) is shown in FIG. 16 and Sequence 22 (ST2) is shown in FIG. 17.

Genetic Code (5′ Upstream Region)
(SEQ ID NO: 11952)
CTTCTGCACTTAAGCACACTATACTTTTTTCACCCAAAGTACCAAATCAA
ACTAGTCAGGATACCTACCTTTGTACAATGTCAGACTCCAGTTAATAACT
CCCCTAGGGCAGAGGGCATATGCACTGATTTACTTTGTACAAATTAACCA
GCATCAGGCAATCAGGCCTGTGCCTAACACATAGTAAGCACTCTATGATT
AAACATCAGTGCTTCGGCTCCAAAGTTTTATTTATTTATTTATTTATTTA
TTTTTTTTTTTTTTGAGACGGAGTCTCGCTCTGTCGCCCAGGCTGGAGTG
CAGTGGTGCGATATCGGCTCACTGCAAGCTCCGCCTCCCGAGTTCACGCT
CTTCTCCTGCCTCAGCCTCCCGAGTAGCTGGGACTACAGACGCCCGCCAC
AACGCCCGGCTACTTTTTTTTGTATTTTTAGTAGAGATGGGGTTTCACCG
TGTTAGCCAGGATGGTCTCGATCTCCTGGCCTCGTGATCCGCGCGTCTGG
GCCTCCCAAAGTGCTGGGATTACAGGCGTGAGCCACCGCGCCCGGCGCCC
CGAAAGTTTTAAAAGCTTCCCCTACAAAAGAACAGAACTGAAATTCCTTG
GTCCTGTATTCAATGTCTTTTGTAAGTAATCACTTCTCCCCTACTTACCC
TCCTAGTCTACCGGGCTACCAGGAATTTTTTTTTTTTTTGGAGACAGGGT
CTCACTCTGTCACCCAGGCTGGAGTGCGGTGGCGGGATCACGGCTCACTG
CAGCCTTAACCCCCGGGGCTTGGGTGATCCTCCCACCTTAGTCTCACCAG
TAGCTGGGACTACAGGTCCACGCCACCAGGCCTGGCTAATTTTTTTTATT
TTTAGGGGAGAGGGAGTTTTACCACGTTGCCCAAGCTGGTCTCAAACTCC
TGGGCTCAAGCAATCCTCCTGCCTCAGCCTCCCAAAGTGCTGGGATTACA
GGCATAAACCACCGCAAATTCTTTACACCTATCAAATTCCACCCATTATT
TGGGACCCAGTTGAAATCCCTCTTTGGCAAAAAGACTTTCTAGACAACTC
CAGGCCTCATAACCTCTCCTTTCTCTGAAGATCTGTAGCATTCAGCCTAG
CACTGTCCAATAGAACGTTCTATGATAACAGAAAAGTTCTACATCTGTAC
TGTATGTTCTTTTATGTAGAACAGCTACCTTGTTAGCACAAGTGTAAAGT
CTCACCATCTCTTTGATGACAACATGTTACATTGGATGGTTAAAACATTT
ATCAGCTCCCCCAGTAGACTGCAATTTCTGTGAACAAGATACAACTTATT
CTTCATAGCAACTCTGACAAAGTTGCAAAAGGTATATATATGTTGGCCAG
GCAAGGTGGTTCACGCCTGTAATCCCAGCACTTTGGGAGGCTGAGGTGGG
CAGATCTCTTGAGGTCAGGAGTTTGAGACCAGCCTGGTCAATATAGTGAA
ACCTTATCTCTACTAAAAATACAAAAATTAGCCGGGCGTAGTGGCGGGCA
CCTGTAATCCCAGCTACTCAGGAGGCTGAGGTGCGAGAATCACTTGAACC
CGGGAGGAGGAGGTTGCAGTGAGCCACGATCATGCCACTGCACTCCAGCC
TGGGTGATAGAGTGCAACTCCAACTCAAAAAAAAAAAAAAAAAAGTATAT
ATTTGTTGATTTGCACATCACCTAAGAAAACCATAAGCTAAGAAGGTTTG
GACTCAGGCGTCTGGAAAGTTGGTCACCACCTCTACCCCACCTCATATCT
GAATGTCAAGAGACACGTAGAGGCAGAGAAGTTAAAGCAACTTTCTAGAG
ACAGAAATGACCACTGATCAAGCCACAATGCACTCTGGTTTAAATGACAT
TTAGGTCATGACTGTCCTTAATCTAAAACAAACCTAGATTAGTATTTCTT
TTCATTAGTAAATAGCTAAATTCTGATGGTAAATTATGCTGACCAAAAAC
AGTTCCTCACTTCCCAAGTTAGACATAGCAATTAGAAAAATAATCTAAGC
AAGCTCCATTTGTATTTCTTTTTTCACCTGTTTATTGAATATTTACCTCC
CATGAAGTCTTTCAGCCTATTGGTGGTATTTTACTGTTCAGATATATGTT
AGAATTTCACTGATACTTACTGGGCGCGGTGGCTCACACCTGTAATCCCA
GCACTTTGGGAGATAGAGGTGGGCAAATCACAAGGTCAGGAGTTCAAGAC
CAGCCTGGCCAATATGGTAAAACCCCGGTCTCTACTAAAAATACAAAAAT
TAGCTGGGCGTGGTGGCGCACGCCTGTAGTCCCAGCTACTTGGGAGGCTG
AGGCAGGAGAATCGCTTGAACCCAGGAGGCAGAGGTTGCAGTGAGCCAAG
ATTGCGCCACTGCACTCTAGCCTGGGCAACAGAGCAAGACTCTGACTCAA
AAAAAAAAAAAAAAAGAATTTCACTGATACTTTTCACAAAATATACAGAA
GGAGGCACAAATTCCACCACTATGGCACTCTGCTGCGTTGGCCAAGTGTC
TTGATCCTTTGGCCTCAATTTTCTTATCTACGATATTAGGGTAATTGTTA
TGTGAACTACCCACCTCACAAGTCCTTTGTGGGTTAATTCATAACTGTGC
TGTGGGTATTTCTTTTTCTTTCCTTTCTTCCTCCTTTCCTTTCTTTCTTT
CTTAAAGATGGGTTCTCATTATGGTGCTTAGACTAGACTCTAGACCCAAT
TCCTGGCCTCTCACCATGTTGCCCAGACCAGACTCAACTCCTGGACTCAA
GGAATCCTCCCACCTCAGCCTTCAATTAGCTGGGATCAGAGGTGTGCACC
ACCATGCCTGGCACTGTGGATATTTCTAAGTGATTATTCTTCTCAAATGA
ACTACATAAAAAACAAAAGATTCATGAATTTACTAATGGTTCTTTGTGAT
GGATGTGCTAATATAGAGACTAAAATCAAGGCTCCAACCTCTAAAACATT
TTTTTTTAAATTCCAGACTTGTTTCCCCATCCCACTGTGCAAACTGAACA
AAAACTGGGCTAGCACTCCTGTCTGGAACATGTAATAAGGAAATAAATGT
GCTGACTCAGAGAACACAGACATATTTAATATAAAATAAGATAGAAAACT
GGCTGAACCAAGTCATAACACAGTCTAAATCCACATATAAAAGATTGAGA
TGATTTTCTGCTTTGCTTTATTCAAGCCCAATGCTTTATCAGCACAGCCA
GCCAAAAATTTACAACCCATACACAGACTATGTAAACCTTTAGTTGCACA
TACAGTAAGACCAGCAGGTACACACTATACACATTTTTAATTAAAAAAAT
GACTAACCACTGATTTTGTCACCACACTTAACAACGACCTGATATGGCAC
AGAGTGATGTGTACCAAACATGGAAATACCAACTTGGGCGACGGTTTGAA
TCTTGTTAACTTCAGTGCAACCACACCCCCCAAATGCATGTAAAGTTTGC
ACACATGGTTTTTTCAAGGCCAGCCTGTCTTTGTTTCCCTCTCCTCTGCA
TTTACCCAAGATCTTGGCTCTGAGACAGAAAACTCCCACTCTCAATTGGT
TCATTCCGTCCTATGCAATTAAGCAACACCACAATCCAGTAAATGCAATG
GCTCAATTATTTATCTTCTGGCCGACTTTACCAGGTATTTGGAAAAGGAC
AATGTCAAGAGGTTTATTTCTCTCTCTAGAGCTGGCTTGACGGGTTGATG
GGGATTTTATTTTGTCTTTTTTTCTCTTTTTTACAAGGCGGGGACGTGGG
GGGAGCATAATTTAACCTAGAAAAAGATGCGAGGGAATTTAGAAAGAGTA
CCGGTCTGTCAATTTCCCTACAGGAAACTTGATTCTTATGCAATAAAGCC
TACCCACGACCAGCCAGCCCGTAAGGCTGCAGGCGACAGACACACCTATT
CCTGCCTCCAAAAGGGCACAGCTGTCTCCTGAAGGAGCGGGAACAGGGCA
AGCGGAGGAAGTGGCTCAGCGGGAGCCGCCGACCGGGCGGGGAGGAGGCG
CTTTCCGACCCCCCACTCGCGCCGGTGATCCCCGTCGGCGTGACAGTCGC
TCCGGTGGCCGGAACGTCCCCAGGGCCCCAGGGAGCAGGAAATCGGGGGA
CTGTCCCTCACTCCTGCCGCCGCAACCGAGTGCGCCCTCGCCCCACGGTG
CCCCCTCGAGCGCGTTCTGTTTCTCCGAAGAACGAAACTTCCCTCCAGCG
CCCCGAGTCCCTTCCGAGGCCCGCTCCTGTCATCCCGAAGAGTCTTCCCT
CAGGGCGACCCTCCGCGTCTCTTCATCTCTCCCGGCCCCACTGCAGCGTC
CATCACAACATCCCCAAGGTCCCAGAGGCCCCCTGCCGCTGCGGAGCCCC
CGGGTCCCCAGGCCTCCCCAACGGCCCCACCCTGCACCCCCTTCACCTGT
TTCTCCGGCAGAGGCCGAGAGGCCGGGGCTGCGCGTGTGCCGGGGACGGG
CGGCGAGGCTCCCTCAGGCCGAAGGGCCTCTCCGAGCCGAGGGGGAGAGA
CAGCGCC

4) HIF1A. Hypoxia-inducible factors (HIFs) are transcription factors that respond to changes in available oxygen in the cellular environment, specifically, to decreases in oxygen, or hypoxia. Hypoxia-inducible factor-1 (HIF-1a) is the alpha subunit of the HIF-1 dimeric transcriptional complex involved in the maintenance of oxygen and energy homoeostasis. Hypoxia often keeps cells from differentiating. However, hypoxia promotes the formation of blood vessels, and is important for the formation of a vascular system in embryos, and cancer tumors. The HIF-1 alpha subunit is oxygen labile and is degraded by the proteasome following prolyl-hydroxylation and ubiquitination in normoxic cells. There is also evidence that HIF-1 is also involved in immune reactions (Hurwig-Burgel et al, J Interferon Cytokine Res. 2005; 25(6):297-310). Immunomodulatory peptides, including interleukin-1 (IL-1) and tumor necrosis factor-alpha (TNF-alpha), stimulate HIF-1 dependent gene expression even in normoxic cells. Both the hypoxic and the cytokine-induced activation of HIF-1 involve the phosphatidylinositol-3-kinase (PI3K) and the mitogen-activated protein kinase (MAPK) signaling pathways. In addition, heat shock proteins (HSP) and other cofactors interact with HIF-1 subunits. HIF-1 blockade may be beneficial to prevent tumor angiogenesis and tumor growth.

Protein: HIF1A Gene: HIF1A (Homo sapiens, chromosome 14, 62162119-62214977 [NCBI Reference Sequence: NC_{—}000014.8]; start site location: 62162523; strand: positive)

Gene Identification

| | |
|---|---|
| GeneID | 3091 |
| HGNC | 4910 |
| HPRD | 04517 |
| MIM | 603348 |

Targeted Sequences

| | | | Relative upstream |
|---|---|---|---|
| Sequence | | | location to gene start |
| ID No: | Design ID | Sequence (5′-3′) | site |
| 992 | HI1 | CAGGCCGGCGCGCGCTCCCGCAA | 390 |
| 1048 | HI2 | GGACGGGCTGCGACGCTCACGTGC | 539 |
| 1089 | | GAGGTGGGGGTGCGAGGCGGGAAACCC | 108 |
| | | CTCG | |
| 1090 | | CAATCGCCGGGGTCCGGGCCCGGC | 162 |
| 1129 | | TGGCCGAAGCGACGAAGAGGG | 232 |
| 1130 | | GGGCGGAGGCGCGCTCGGGCGCG | 325 |
| 1142 | | CACGGCGGGCGGCCCCCAGGCTCGC | 26 |
| 1214 | | CAGGCCGGCGCGCGCTCCCGCAAGCCCG | 390 |
| 1270 | | CGATTGCCGCCCAACTCTGCTGGG | 789 |

Target Shift Sequences

| | | Relative |
|---|---|---|
| | | upstream |
| | | location to |
| Sequence | | gene start |
| ID No: | Sequence (5′-3′) | site |
| 992 | CAGGCCGGCGCGCGCTCCCGCAA | 390 |
| 993 | AGGCCGGCGCGCGCTCCCGC | 391 |
| 994 | GGCCGGCGCGCGCTCCCGCA | 392 |
| 995 | GCCGGCGCGCGCTCCCGCAA | 393 |
| 996 | CCGGCGCGCGCTCCCGCAAG | 394 |
| 997 | CGGCGCGCGCTCCCGCAAGC | 395 |
| 998 | GGCGCGCGCTCCCGCAAGCC | 396 |
| 999 | GCGCGCGCTCCCGCAAGCCC | 397 |
| 1000 | CGCGCGCTCCCGCAAGCCCG | 398 |
| 1001 | GCGCGCTCCCGCAAGCCCGC | 399 |
| 1002 | CGCGCTCCCGCAAGCCCGCC | 400 |
| 1003 | GCGCTCCCGCAAGCCCGCCT | 401 |
| 1004 | CGCTCCCGCAAGCCCGCCTC | 402 |
| 1005 | GCTCCCGCAAGCCCGCCTCA | 403 |
| 1006 | CTCCCGCAAGCCCGCCTCAC | 404 |
| 1007 | TCCCGCAAGCCCGCCTCACC | 405 |
| 1008 | CCCGCAAGCCCGCCTCACCT | 406 |
| 1009 | CCGCAAGCCCGCCTCACCTG | 407 |
| 1010 | CGCAAGCCCGCCTCACCTGA | 408 |
| 1011 | GCAAGCCCGCCTCACCTGAG | 409 |
| 1012 | CAAGCCCGCCTCACCTGAGG | 410 |
| 1013 | AAGCCCGCCTCACCTGAGGT | 411 |
| 1014 | AGCCCGCCTCACCTGAGGTG | 412 |
| 1015 | GCCCGCCTCACCTGAGGTGG | 413 |
| 1016 | CCCGCCTCACCTGAGGTGGA | 414 |
| 1017 | CCGCCTCACCTGAGGTGGAG | 415 |
| 1018 | CGCCTCACCTGAGGTGGAGG | 416 |
| 1019 | CCAGGCCGGCGCGCGCTCCC | 389 |
| 1020 | CCCAGGCCGGCGCGCGCTCC | 388 |
| 1021 | GCCCAGGCCGGCGCGCGCTC | 387 |
| 1022 | TGCCCAGGCCGGCGCGCGCT | 386 |
| 1023 | CTGCCCAGGCCGGCGCGCGC | 385 |
| 1024 | CCTGCCCAGGCCGGCGCGCG | 384 |
| 1025 | GCCTGCCCAGGCCGGCGCGC | 383 |
| 1026 | CGCCTGCCCAGGCCGGCGCG | 382 |
| 1027 | TCGCCTGCCCAGGCCGGCGC | 381 |
| 1028 | CTCGCCTGCCCAGGCCGGCG | 380 |
| 1029 | GCTCGCCTGCCCAGGCCGGC | 379 |
| 1030 | CGCTCGCCTGCCCAGGCCGG | 378 |
| 1031 | CCGCTCGCCTGCCCAGGCCG | 377 |
| 1032 | CCCGCTCGCCTGCCCAGGCC | 376 |
| 1033 | GCCCGCTCGCCTGCCCAGGC | 375 |
| 1034 | CGCCCGCTCGCCTGCCCAGG | 374 |
| 1035 | GCGCCCGCTCGCCTGCCCAG | 373 |
| 1036 | CGCGCCCGCTCGCCTGCCCA | 372 |
| 1037 | GCGCGCCCGCTCGCCTGCCC | 371 |
| 1038 | AGCGCGCCCGCTCGCCTGCC | 370 |
| 1039 | GAGCGCGCCCGCTCGCCTGC | 369 |
| 1040 | GGAGCGCGCCCGCTCGCCTG | 368 |
| 1041 | GGGAGCGCGCCCGCTCGCCT | 367 |
| 1042 | CGGGAGCGCGCCCGCTCGCC | 366 |
| 1043 | GCGGGAGCGCGCCCGCTCGC | 365 |
| 1044 | GGCGGGAGCGCGCCCGCTCG | 364 |
| 1045 | GGGCGGGAGCGCGCCCGCTC | 363 |
| 1046 | GGGGCGGGAGCGCGCCCGCT | 362 |
| 1047 | GGGGGCGGGAGCGCGCCCGC | 361 |
| 1048 | GGACGGGCTGCGACGCTCACGTGC | 539 |
| 1049 | GACGGGCTGCGACGCTCACG | 540 |
| 1050 | ACGGGCTGCGACGCTCACGT | 541 |
| 1051 | CGGGCTGCGACGCTCACGTG | 542 |
| 1052 | GGGCTGCGACGCTCACGTGC | 543 |
| 1053 | GGCTGCGACGCTCACGTGCT | 544 |
| 1054 | GCTGCGACGCTCACGTGCTC | 545 |
| 1055 | CTGCGACGCTCACGTGCTCG | 546 |
| 1056 | TGCGACGCTCACGTGCTCGT | 547 |
| 1057 | GCGACGCTCACGTGCTCGTC | 548 |
| 1058 | CGACGCTCACGTGCTCGTCT | 549 |
| 1059 | GACGCTCACGTGCTCGTCTG | 550 |
| 1060 | ACGCTCACGTGCTCGTCTGT | 551 |
| 1061 | CGCTCACGTGCTCGTCTGTG | 552 |
| 1062 | GCTCACGTGCTCGTCTGTGT | 553 |
| 1063 | CTCACGTGCTCGTCTGTGTT | 554 |
| 1064 | TCACGTGCTCGTCTGTGTTT | 555 |
| 1065 | CACGTGCTCGTCTGTGTTTA | 556 |
| 1066 | ACGTGCTCGTCTGTGTTTAG | 557 |
| 1067 | CGTGCTCGTCTGTGTTTAGC | 558 |
| 1068 | GTGCTCGTCTGTGTTTAGCG | 559 |
| 1069 | TGCTCGTCTGTGTTTAGCGG | 560 |
| 1070 | GCTCGTCTGTGTTTAGCGGC | 561 |
| 1071 | CTCGTCTGTGTTTAGCGGCG | 562 |
| 1072 | TCGTCTGTGTTTAGCGGCGG | 563 |
| 1073 | CGTCTGTGTTTAGCGGCGGA | 564 |
| 1074 | GTCTGTGTTTAGCGGCGGAG | 565 |
| 1075 | TCTGTGTTTAGCGGCGGAGG | 566 |
| 1076 | CTGTGTTTAGCGGCGGAGGA | 567 |
| 1077 | TGTGTTTAGCGGCGGAGGAA | 568 |
| 1078 | GGGACGGGCTGCGACGCTCA | 538 |
| 1079 | TGGGACGGGCTGCGACGCTC | 537 |
| 1080 | CTGGGACGGGCTGCGACGCT | 536 |
| 1081 | GCTGGGACGGGCTGCGACGC | 535 |
| 1082 | AGCTGGGACGGGCTGCGACG | 534 |
| 1083 | CAGCTGGGACGGGCTGCGAC | 533 |
| 1084 | ACAGCTGGGACGGGCTGCGA | 532 |
| 1085 | CACAGCTGGGACGGGCTGCG | 531 |
| 1086 | GCACAGCTGGGACGGGCTGC | 530 |
| 1087 | GGCACAGCTGGGACGGGCTG | 529 |
| 1088 | AGGCACAGCTGGGACGGGCT | 528 |
| 1089 | GAGGTGGGGGTGCGAGGCGGGAAACCCCTCG | 108 |
| 1090 | CAATCGCCGGGGTCCGGGCCCGGC | 162 |
| 1091 | AATCGCCGGGGTCCGGGCCC | 163 |
| 1092 | ATCGCCGGGGTCCGGGCCCG | 164 |
| 1093 | TCGCCGGGGTCCGGGCCCGG | 165 |
| 1094 | CGCCGGGGTCCGGGCCCGGC | 166 |
| 1095 | GCCGGGGTCCGGGCCCGGCT | 167 |
| 1096 | CCGGGGTCCGGGCCCGGCTC | 168 |
| 1097 | CGGGGTCCGGGCCCGGCTCC | 169 |
| 1098 | GGGGTCCGGGCCCGGCTCCG | 170 |
| 1099 | GGGTCCGGGCCCGGCTCCGA | 171 |
| 1100 | GGTCCGGGCCCGGCTCCGAG | 172 |
| 1101 | GTCCGGGCCCGGCTCCGAGC | 173 |
| 1102 | TCCGGGCCCGGCTCCGAGCC | 174 |
| 1103 | CCGGGCCCGGCTCCGAGCCT | 175 |
| 1104 | CGGGCCCGGCTCCGAGCCTC | 176 |
| 1105 | GGGCCCGGCTCCGAGCCTCT | 177 |
| 1106 | GGCCCGGCTCCGAGCCTCTC | 178 |
| 1107 | GCCCGGCTCCGAGCCTCTCC | 179 |
| 1108 | CCCGGCTCCGAGCCTCTCCT | 180 |
| 1109 | CCGGCTCCGAGCCTCTCCTC | 181 |
| 1110 | CGGCTCCGAGCCTCTCCTCA | 182 |
| 1111 | GGCTCCGAGCCTCTCCTCAG | 183 |
| 1112 | GCTCCGAGCCTCTCCTCAGG | 184 |
| 1113 | CTCCGAGCCTCTCCTCAGGT | 185 |
| 1114 | TCCGAGCCTCTCCTCAGGTG | 186 |
| 1115 | CCGAGCCTCTCCTCAGGTGG | 187 |
| 1116 | CGAGCCTCTCCTCAGGTGGC | 188 |
| 1117 | GCAATCGCCGGGGTCCGGGC | 161 |
| 1118 | GGCAATCGCCGGGGTCCGGG | 160 |
| 1119 | CGGCAATCGCCGGGGTCCGG | 159 |
| 1120 | GCGGCAATCGCCGGGGTCCG | 158 |
| 1121 | GGCGGCAATCGCCGGGGTCC | 157 |
| 1122 | GGGCGGCAATCGCCGGGGTC | 156 |
| 1123 | CGGGCGGCAATCGCCGGGGT | 155 |
| 1124 | GCGGGCGGCAATCGCCGGGG | 154 |
| 1125 | AGCGGGCGGCAATCGCCGGG | 153 |
| 1126 | AAGCGGGCGGCAATCGCCGG | 152 |
| 1127 | GAAGCGGGCGGCAATCGCCG | 151 |
| 1128 | AGAAGCGGGCGGCAATCGCC | 150 |
| 1129 | TGGCCGAAGCGACGAAGAGGG | 232 |
| 1130 | GGGCGGAGGCGCGCTCGGGCGCG | 325 |
| 1131 | GGCGGAGGCGCGCTCGGGCG | 326 |
| 1132 | GCGGAGGCGCGCTCGGGCGC | 327 |
| 1133 | CGGAGGCGCGCTCGGGCGCG | 328 |
| 1134 | GGAGGCGCGCTCGGGCGCGC | 329 |
| 1135 | GAGGCGCGCTCGGGCGCGCG | 330 |
| 1136 | AGGCGCGCTCGGGCGCGCGG | 331 |
| 1137 | GGCGCGCTCGGGCGCGCGGG | 332 |
| 1138 | GCGCGCTCGGGCGCGCGGGG | 333 |
| 1139 | CGCGCTCGGGCGCGCGGGGA | 334 |
| 1140 | GCGCTCGGGCGCGCGGGGAG | 335 |
| 1141 | CGCTCGGGCGCGCGGGGAGG | 336 |
| 1142 | CACGGCGGGCGGCCCCCAGGCTCGC | 26 |
| 1143 | ACGGCGGGCGGCCCCCAGGC | 27 |
| 1144 | CGGCGGGCGGCCCCCAGGCT | 28 |
| 1145 | GGCGGGCGGCCCCCAGGCTC | 29 |
| 1146 | GCGGGCGGCCCCCAGGCTCG | 30 |
| 1147 | CGGGCGGCCCCCAGGCTCGC | 31 |
| 1148 | GGGCGGCCCCCAGGCTCGCT | 32 |
| 1149 | GGCGGCCCCCAGGCTCGCTC | 33 |
| 1150 | GCGGCCCCCAGGCTCGCTCC | 34 |
| 1151 | CGGCCCCCAGGCTCGCTCCG | 35 |
| 1152 | GGCCCCCAGGCTCGCTCCGG | 36 |
| 1153 | GCCCCCAGGCTCGCTCCGGC | 37 |
| 1154 | CCCCCAGGCTCGCTCCGGCC | 38 |
| 1155 | CCCCAGGCTCGCTCCGGCCT | 39 |
| 1156 | CCCAGGCTCGCTCCGGCCTA | 40 |
| 1157 | CCAGGCTCGCTCCGGCCTAA | 41 |
| 1158 | CAGGCTCGCTCCGGCCTAAG | 42 |
| 1159 | AGGCTCGCTCCGGCCTAAGC | 43 |
| 1160 | GGCTCGCTCCGGCCTAAGCG | 44 |
| 1161 | GCTCGCTCCGGCCTAAGCGC | 45 |
| 1162 | CTCGCTCCGGCCTAAGCGCT | 46 |
| 1163 | TCGCTCCGGCCTAAGCGCTG | 47 |
| 1164 | CGCTCCGGCCTAAGCGCTGG | 48 |
| 1165 | GCTCCGGCCTAAGCGCTGGC | 49 |
| 1166 | CTCCGGCCTAAGCGCTGGCT | 50 |
| 1167 | TCCGGCCTAAGCGCTGGCTC | 51 |
| 1168 | CCGGCCTAAGCGCTGGCTCC | 52 |
| 1169 | CGGCCTAAGCGCTGGCTCCC | 53 |
| 1170 | GGCCTAAGCGCTGGCTCCCT | 54 |
| 1171 | GCCTAAGCGCTGGCTCCCTC | 55 |
| 1172 | CCTAAGCGCTGGCTCCCTCC | 56 |
| 1173 | CTAAGCGCTGGCTCCCTCCA | 57 |
| 1174 | TAAGCGCTGGCTCCCTCCAC | 58 |
| 1175 | AAGCGCTGGCTCCCTCCACA | 59 |
| 1176 | AGCGCTGGCTCCCTCCACAC | 60 |
| 1177 | GCGCTGGCTCCCTCCACACG | 61 |
| 1178 | CGCTGGCTCCCTCCACACGC | 62 |
| 1179 | GCTGGCTCCCTCCACACGCG | 63 |
| 1180 | CTGGCTCCCTCCACACGCGG | 64 |
| 1181 | TGGCTCCCTCCACACGCGGA | 65 |
| 1182 | GGCTCCCTCCACACGCGGAG | 66 |
| 1183 | GCTCCCTCCACACGCGGAGA | 67 |
| 1184 | CTCCCTCCACACGCGGAGAA | 68 |
| 1185 | TCCCTCCACACGCGGAGAAG | 69 |
| 1186 | CCCTCCACACGCGGAGAAGA | 70 |
| 1187 | CCTCCACACGCGGAGAAGAG | 71 |
| 1188 | CTCCACACGCGGAGAAGAGA | 72 |
| 1189 | TCACGGCGGGCGGCCCCCAG | 25 |
| 1190 | TTCACGGCGGGCGGCCCCCA | 24 |
| 1191 | CTTCACGGCGGGCGGCCCCC | 23 |
| 1192 | TCTTCACGGCGGGCGGCCCC | 22 |
| 1193 | GTCTTCACGGCGGGCGGCCC | 21 |
| 1194 | TGTCTTCACGGCGGGCGGCC | 20 |
| 1195 | ATGTCTTCACGGCGGGCGGC | 19 |
| 1196 | GATGTCTTCACGGCGGGCGG | 18 |
| 1197 | CGATGTCTTCACGGCGGGCG | 17 |
| 1198 | GCGATGTCTTCACGGCGGGC | 16 |
| 1199 | CGCGATGTCTTCACGGCGGG | 15 |
| 1200 | CCGCGATGTCTTCACGGCGG | 14 |
| 1201 | CCCGCGATGTCTTCACGGCG | 13 |
| 1202 | CCCCGCGATGTCTTCACGGC | 12 |
| 1203 | TCCCCGCGATGTCTTCACGG | 11 |
| 1204 | GTCCCCGCGATGTCTTCACG | 10 |
| 1205 | GGTCCCCGCGATGTCTTCAC | 9 |
| 1206 | CGGTCCCCGCGATGTCTTCA | 8 |
| 1207 | TCGGTCCCCGCGATGTCTTC | 7 |
| 1208 | ATCGGTCCCCGCGATGTCTT | 6 |
| 1209 | AATCGGTCCCCGCGATGTCT | 5 |
| 1210 | GAATCGGTCCCCGCGATGTC | 4 |
| 1211 | TGAATCGGTCCCCGCGATGT | 3 |
| 1212 | GTGAATCGGTCCCCGCGATG | 2 |
| 1213 | GGTGAATCGGTCCCCGCGAT | 1 |
| 1214 | CAGGCCGGCGCGCGCTCCCGCAAGCCCG | 390 |
| 1215 | AGGCCGGCGCGCGCTCCCGC | 391 |
| 1216 | GGCCGGCGCGCGCTCCCGCA | 392 |
| 1217 | GCCGGCGCGCGCTCCCGCAA | 393 |
| 1218 | CCGGCGCGCGCTCCCGCAAG | 394 |
| 1219 | CGGCGCGCGCTCCCGCAAGC | 395 |
| 1220 | GGCGCGCGCTCCCGCAAGCC | 396 |
| 1221 | GCGCGCGCTCCCGCAAGCCC | 397 |
| 1222 | CGCGCGCTCCCGCAAGCCCG | 398 |
| 1223 | GCGCGCTCCCGCAAGCCCGC | 399 |
| 1224 | CGCGCTCCCGCAAGCCCGCC | 400 |
| 1225 | GCGCTCCCGCAAGCCCGCCT | 401 |
| 1226 | CGCTCCCGCAAGCCCGCCTC | 402 |
| 1227 | GCTCCCGCAAGCCCGCCTCA | 403 |
| 1228 | CTCCCGCAAGCCCGCCTCAC | 404 |
| 1229 | TCCCGCAAGCCCGCCTCACC | 405 |
| 1230 | CCCGCAAGCCCGCCTCACCT | 406 |
| 1231 | CCGCAAGCCCGCCTCACCTG | 407 |
| 1232 | CGCAAGCCCGCCTCACCTGA | 408 |
| 1233 | GCAAGCCCGCCTCACCTGAG | 409 |
| 1234 | CAAGCCCGCCTCACCTGAGG | 410 |
| 1235 | AAGCCCGCCTCACCTGAGGT | 411 |
| 1236 | AGCCCGCCTCACCTGAGGTG | 412 |
| 1237 | GCCCGCCTCACCTGAGGTGG | 413 |
| 1238 | CCCGCCTCACCTGAGGTGGA | 414 |
| 1239 | CCGCCTCACCTGAGGTGGAG | 415 |
| 1240 | CGCCTCACCTGAGGTGGAGG | 416 |
| 1241 | CCAGGCCGGCGCGCGCTCCC | 389 |
| 1242 | CCCAGGCCGGCGCGCGCTCC | 388 |
| 1243 | GCCCAGGCCGGCGCGCGCTC | 387 |
| 1244 | TGCCCAGGCCGGCGCGCGCT | 386 |
| 1245 | CTGCCCAGGCCGGCGCGCGC | 385 |
| 1246 | CCTGCCCAGGCCGGCGCGCG | 384 |
| 1247 | GCCTGCCCAGGCCGGCGCGC | 383 |
| 1248 | CGCCTGCCCAGGCCGGCGCG | 382 |
| 1249 | TCGCCTGCCCAGGCCGGCGC | 381 |
| 1250 | CTCGCCTGCCCAGGCCGGCG | 380 |
| 1251 | GCTCGCCTGCCCAGGCCGGC | 379 |
| 1252 | CGCTCGCCTGCCCAGGCCGG | 378 |
| 1253 | CCGCTCGCCTGCCCAGGCCG | 377 |
| 1254 | CCCGCTCGCCTGCCCAGGCC | 376 |
| 1255 | GCCCGCTCGCCTGCCCAGGC | 375 |
| 1256 | CGCCCGCTCGCCTGCCCAGG | 374 |
| 1257 | GCGCCCGCTCGCCTGCCCAG | 373 |
| 1258 | CGCGCCCGCTCGCCTGCCCA | 372 |
| 1259 | GCGCGCCCGCTCGCCTGCCC | 371 |
| 1260 | AGCGCGCCCGCTCGCCTGCC | 370 |
| 1261 | GAGCGCGCCCGCTCGCCTGC | 369 |
| 1262 | GGAGCGCGCCCGCTCGCCTG | 368 |
| 1263 | GGGAGCGCGCCCGCTCGCCT | 367 |
| 1264 | CGGGAGCGCGCCCGCTCGCC | 366 |
| 1265 | GCGGGAGCGCGCCCGCTCGC | 365 |
| 1266 | GGCGGGAGCGCGCCCGCTCG | 364 |
| 1267 | GGGCGGGAGCGCGCCCGCTC | 363 |
| 1268 | GGGGCGGGAGCGCGCCCGCT | 362 |
| 1269 | GGGGGCGGGAGCGCGCCCGC | 361 |
| 1270 | CGATTGCCGCCCAACTCTGCTGGG | 789 |
| 1271 | GATTGCCGCCCAACTCTGCT | 790 |
| 1272 | ATTGCCGCCCAACTCTGCTG | 791 |
| 1273 | TTGCCGCCCAACTCTGCTGG | 792 |
| 1274 | TGCCGCCCAACTCTGCTGGG | 793 |
| 1275 | GCCGCCCAACTCTGCTGGGC | 794 |
| 1276 | CCGCCCAACTCTGCTGGGCT | 795 |
| 1277 | CGCCCAACTCTGCTGGGCTC | 796 |
| 1278 | ACGATTGCCGCCCAACTCTG | 788 |
| 1279 | CACGATTGCCGCCCAACTCT | 787 |
| 1280 | GCACGATTGCCGCCCAACTC | 786 |
| 1281 | GGCACGATTGCCGCCCAACT | 785 |
| 1282 | GGGCACGATTGCCGCCCAAC | 784 |
| 1283 | TGGGCACGATTGCCGCCCAA | 783 |
| 1284 | CTGGGCACGATTGCCGCCCA | 782 |
| 1285 | GCTGGGCACGATTGCCGCCC | 781 |
| 1286 | TGCTGGGCACGATTGCCGCC | 780 |
| 1287 | GTGCTGGGCACGATTGCCGC | 779 |
| 1288 | AGTGCTGGGCACGATTGCCG | 778 |
| 1289 | CAGTGCTGGGCACGATTGCC | 777 |
| 1290 | TCAGTGCTGGGCACGATTGC | 776 |
| 1291 | CTCAGTGCTGGGCACGATTG | 775 |
| 1292 | CCTCAGTGCTGGGCACGATT | 774 |
| 1293 | GCCTCAGTGCTGGGCACGAT | 773 |
| 1294 | GGCCTCAGTGCTGGGCACGA | 772 |
| 1295 | CGGCCTCAGTGCTGGGCACG | 771 |
| 1296 | TCGGCCTCAGTGCTGGGCAC | 770 |
| 1297 | CTCGGCCTCAGTGCTGGGCA | 769 |
| 1298 | CCTCGGCCTCAGTGCTGGGC | 768 |
| 1299 | TCCTCGGCCTCAGTGCTGGG | 767 |
| 1300 | CTCCTCGGCCTCAGTGCTGG | 766 |
| 1301 | TCTCCTCGGCCTCAGTGCTG | 765 |
| 1302 | TTCTCCTCGGCCTCAGTGCT | 764 |
| 1303 | TTTCTCCTCGGCCTCAGTGC | 763 |
| 1304 | CTTTCTCCTCGGCCTCAGTG | 762 |
| 1305 | TCTTTCTCCTCGGCCTCAGT | 761 |
| 1306 | CTCTTTCTCCTCGGCCTCAG | 760 |
| 1307 | TCTCTTTCTCCTCGGCCTCA | 759 |
| 1308 | CTCTCTTTCTCCTCGGCCTC | 758 |
| 1309 | GCTCTCTTTCTCCTCGGCCT | 757 |
| 1310 | TGCTCTCTTTCTCCTCGGCC | 756 |
| 1311 | CTGCTCTCTTTCTCCTCGGC | 755 |
| 1312 | CCTGCTCTCTTTCTCCTCGG | 754 |
| 1313 | TCCTGCTCTCTTTCTCCTCG | 753 |

Hot Zones (Relative upstream location to gene start site)
1-1050
1500-1700
2000-2450

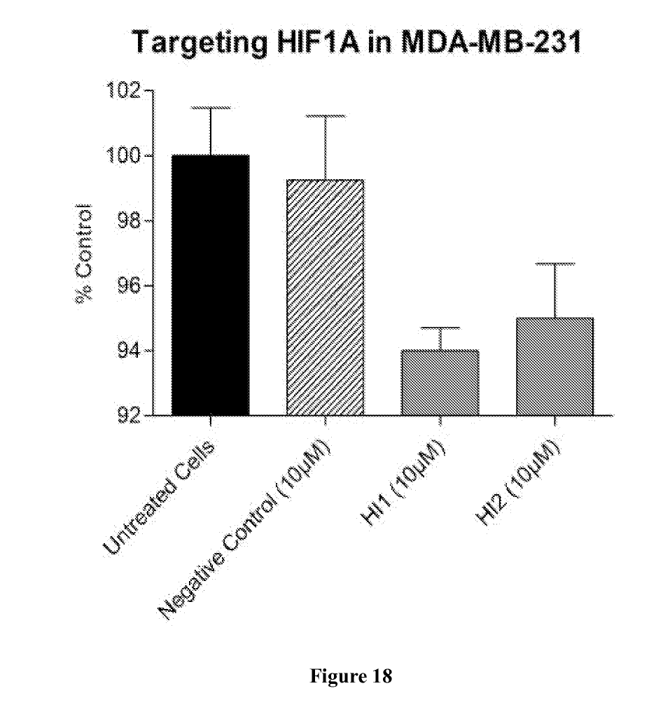

FIG. 18 shows MDA-MB-231 (human breast cell line), HI1 (31) and HI2 (32) at 10 μM showed increased inhibition compared to the untreated control and the negative control. The HIF1A sequences HI1 (31) and HI2 (32) (shown below) fit the independent and dependent DNAi motif claims.

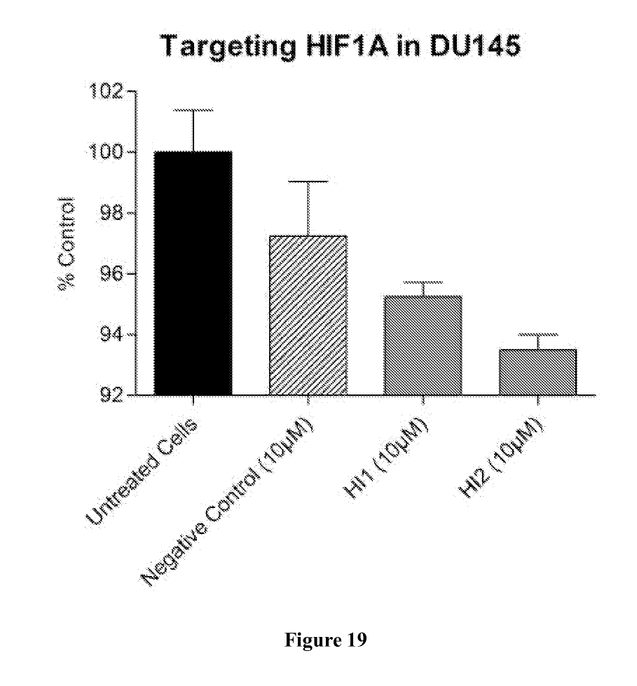

FIG. 19 shows DU145 (human prostate cell line), HI1 (31) and HI2 (32) at 10 μM produced statistically significant (P<0.05) inhibition compared to the untreated control values. The negative control inhibition values did not a produce statistically significant difference compared to the untreated control values. The HIF1A sequences HI1 (31) and HI2 (32) (shown below) fit the independent and dependent DNAi motif claims.

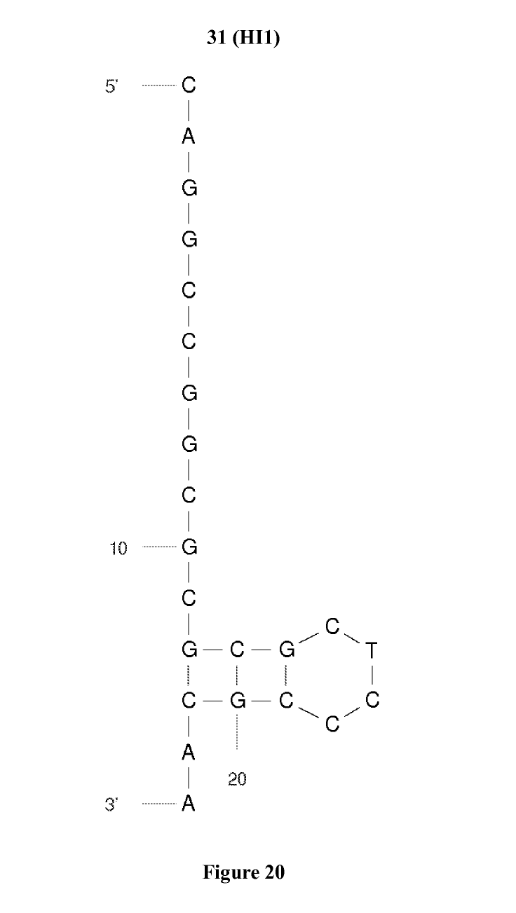

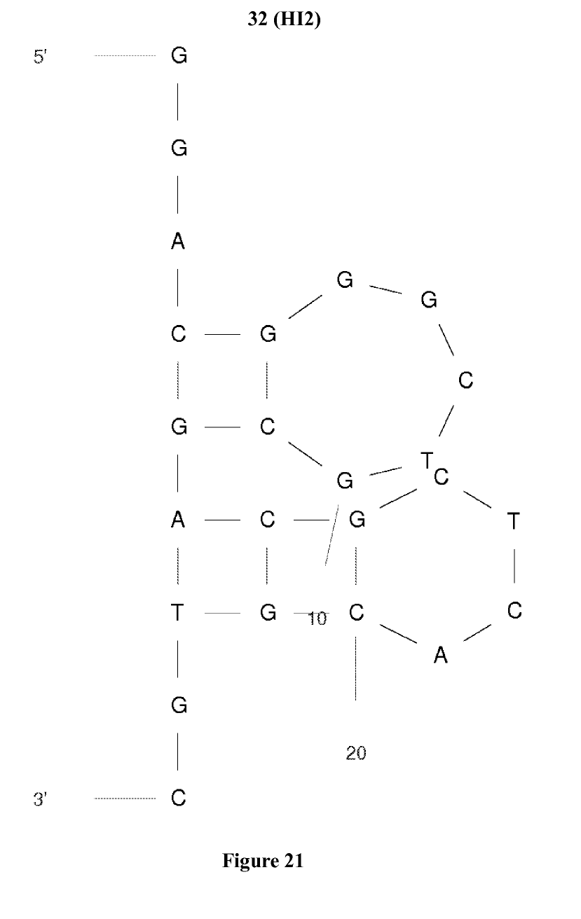

The secondary structures for HI1 and HI2 are shown in FIGS. 20 and 21. Sequence 31 (HI1) is shown in FIG. 20 and Sequence 32 (HI2) is shown in FIG. 21.

Genetic Code (5′ Upstream Region)
(SEQ ID NO: 11953)
GTTTCCCTTGAGGCCAGGTCTTGTTAAGAAGAACAGAGAGCCCTGAGAGT
ATTTCACGATGGTTACTTACCCCTTCTCCCTGGCAAAAGCAAAGCAGATT
TTTCTCAGATCTTTACAGTGAGAATCTGACAGGATTCACAGAGGTAAAAC
TGAGGTAAGTATTGAGGCCCCTCTCAGACTGAGCCTCCTTGGAGTTTTTT
AACTCTCAAGCTAGTCTGCACTGAGCCTCCAGCAATTCCCCAATTACAGT
TTAGTGTTCCTACTGATGTTGGCTCCAGCTGTGAAACTAGCTTCAGCTTC
TGGCTTCTGTGCCTGGGCTCTGCTCCTGGTAAACTGTGATTCTCTGAAAA
GCTGTGATTCTCTGTATCTATCTGTCTGTCTCTCTAGTTTTTAGGGCAGT
GGTTTTTCCTGTGACCTCAATTCTCTGGTGGATCTAAGAAGAGTTACTGA
TTTCAGTTTGCTTAGCTTTTTTTTTTCTTGTTGTGAGGATGGGAGTGACA
ACTTCCAAGCTCTTTACATGTTGAACAGGAAACTGAAAGCCCCTTGGTGT
TCCTTTGTAAATTCATCTTAAAAATATTTATCATAATTGAAAAGTGCTAA
TATCAAATTTTCAGTCTGTTTATATTCCCCCTAAACTCAGATAAATATAC
ATTTTATTTTGTGTGTCTGTGTGTGTGGGTTTTGTTGTTGTTTGTTTTTT
GTGTTTTTTTTTAAGATATAGGGTCTTGCTCTGTCAAGGCTGGAGTGCAG
TGGCACAATCGTACATCTCTGCAGCCTCGAACTCCTGGGAGAAAGTGATC
CTCCCGCTTTAGCCTTCAGAGTAGCTACGACTACAGGCACTAACCACCAA
GCCCAGCTAATTTTTAAAATTTTTTGTAGAGATGGGAGTTTCACTTTGTT
GCCCAGGCTGGTCTCAAACTCTTGGCCTCAAGTGATCCTCCTGCCTCAGC
CTCCCAAAATGTTGGAATCACAGATACTTTGTGTCTTGATTCTTGAAAGG
AAAAAACAAAGATTTTTAATGCCTCTTATCTTGTACGCACTTTCCTTCCA
AACAATACCCTTTTGCTGCCATTGTTCTCGTTATGAATAGCTTAAAGAAA
AAGAAACAACTAAGGGTAGTAATAGGCCAGGAATCACTTACTGAATACTA
GGTCTTCTTGTATAGTTTGATACCCTATAAATTGTGTGCATCTGATGCAT
TTCACCTTCAAAAGGCTCAATGCTCTGTATTATTTAGTAGTAATCAAAAT
TTCAAGTTTTACTTAACCTCCTGATTCACTGCCCAATTTCCTAATAAATA
CGGGCTAAGGGTCAATGGGGTCATTTGCAAGTAATCTTGTAGTCTACTCA
GAAAGTTCTGCAAAGTTAGAAAGTGATTAAATGACTGTTTGTTAAGATAT
ACTTACATAGTAATAACCTAAATGCATTTGTTAAGTGGTTGTAGAGAGAG
GGATTTAAAATTTTATCCTATATGAAATTTTCCTTTTTGGTGTCTGTTAT
TTAATAGGATTGTTTGAATTAGGGGATACTATTTGGTGCCTTTGTAACTA
TATGAAAATTAGTTGGTTGAATATTACTGCTTTCCATGTTCATATTTATA
TTTGTATAGACATATATATATATACACATATACTACTTTCCTTTCCATTT
TCATATTTATATTTGTGTATACACATATACATAAACATATATTTTATACA
TTTTTGAAAAGGAAAATTAACTTAAGGGCATATTTAATGAATATTCAAAA
ATTTTTTTGCTGATCAAATTATCATTCTGCTTTAAACTTTTGAAATGATC
CAAAAAAATTTTAAATGACTTAGATTTACTGTTACAAAATGCTTGTCTTT
TGATGTCACAAACATTATATACTATAATCACTGGCCAGAGATAATTGCTA
TAAGTATAATGAAAAGGGAAATGATGGAAGAATCTCTGCAGCTATCCTCA
TAAATGAGGGTGGGAACACGATGGGCAGTTCCAAAGTTGAAAATAGAGAA
TATATGTGGATTTATATTAACATAATTGGTATTCTTGGATAGTTAAAAAT
GGCTAAACTGTAGGAGAAGCCCGAGTAATTACTGTTAACAGAGGAATAAA
TTTGAGGGCAATAATAATGATGATAGGCCAGGCACTGTGGCTCATGCCTG
TAATCCCAGCACTTTGGGAACCCGAGGCGAGCGGACCACCTGAGGTCAGG
AGTTCGAGAGCAGCCTGGCCAACATGGTGAAACCTCGTCTCTACTAAAAA
TAGAAAAATTATCCGAGTGTGGTGGTGCGTGCCTGTAATCCCAGCTACTT
GGGAGGCTGAGGCAGGAGAATCACTTGTACCTGGGAGGCGGAGTTGCAGT
GAGCCGAAATCGCGCCACTGCGCTCCAGCCTGTGGGCCAGAGCGAGACTC
CGCCTCAGAATAATAATAATGATAATAATAATAACGCCACCAACAATACT
AAGAGCTAACATTTACTGAGTGCTTACTATGCACCAGATATTGTTCTAAG
TATACATTTATTATCTCATTTAACCATCCATAATACTGTGGTATAGACAC
TTTTATATCCATTTTATAAATAAGTAAACTGAGTTATGGAGAGATTAAAC
GACTTGCCAGTAAGATTCAAAGCCTGTGTACAAGCTCACGCTTGATTCTG
GAGCCAGTGTTCTTAACACAGTATCTTGAGAATGTTAAACTAAAAAGTTT
TTAATTTACAGTATTCTTTCCACAATTAAAAAAGAAATTATGAGTAATTA
TTTTTAGTTCTTTCTTCTCTTCAGGCATTTCCCATGGTTCTTTTCAAGAC
ATAATACATATCATTTAGTGTTGTAGATCTGAAAAAACAAAAGTAGCGTG
AAGATCAAAAATTTTCTAAAGAGACGGAGTCTCGCTACGTTCCCTAGGCT
GGAACACCCAGGCTTCTCCAGCCTCACACCTCTGAGTAGCTGGAACCACC
CTGTCCGCTAAGGTCAATGTTTAATCGTATCTTTGTAGGTCTACTGACCA
GTTAAAAAGAGGTGCTGTATACATTGGTTGTTGTCTTGTCAGAGTTTGAT
GCTTCTATATAGACCATTGTTTTTACATGCTAATACAATTGAAAGCCACT
ACAGATATTTATATTTACAACCCAAAGCTAGGTTTTAACAAGAAACTCAT
AAGGCAAAGGTGAGAAGTAAAATAATTTAGCGCCAAGTGGAGATATATGT
GCAATGCTACTTTGTTGGGCTCAAAACATATTTTTCTTTTAGAAGACTGA
CAGGCTTGAAGTTTATGCCTCCAAAGACAAAAGTGATTATGTTTTGTTTA
GTAGCTTGCAAAGTTGCCAAAGCCATTTTTTCTACTCTTTCCCTGAAATT
GGTTTATATGCTTATTAAAGTCATTTATACCTATTTGCAAATGCTTAACA
TAGTTTCAGATTTTAAGATTTCCCTGCAACTTTATTTCCCTTGAAGTTTA
CAGCAACAGGAGTTCATTTTTATTTTTAATTGCATTTATTCAGTAAGTAA
ACTCCGCCACAGAAAAACTTAGTAGACAAGGTGAGTTCCCCTGTGCTCCG
TGGCAAAGAGTGCGGTGGGTGACATTGACCCATGGTTAGGTAATCTGGTA
AGGAAAGACCCCGTTGTAACACATCTGAGCAACGAGACCAAAGGAAGGGC
TTGCTGCCACGAGGCGAAGTCTGCTTTTTTGAACAGAGAGCCCAGCAGAG
TTGGGCGGCAATCGTGCCCAGCACTGAGGCCGAGGAGAAAGAGAGCAGGA
GCATTACATTACTGCACCAAGAGTAGGAAAATATGATGCATGTTTGGGAC
CAGGCAACCGAAATCCCTTCTCAGCAGCGCCTCCCAAAGCCGGGCACCGC
CTTCCTTCGGAGAAGGCGCAGAGTCCCCAGACTCGGGCTGAGCCGCACCC
CCATCTCCTTTCTCTTTCCTCCGCCGCTAAACACAGACGAGCACGTGAGC
GTCGCAGCCCGTCCCAGCTGTGCCTCAGCTGACCGCCTCCTGATTGGCTG
AGAGCGGCGTGGGCTGGGGTGGGGACTTGCCGCCTGCGTCGCTCGCCATT
GGATCTCGAGGAACCCGCCTCCACCTCAGGTGAGGCGGGCTTGCGGGAGC
GCGCGCCGGCCTGGGCAGGCGAGCGGGCGCGCTCCCGCCCCCTCTCCCCT
CCCCGCGCGCCCGAGCGCGCCTCCGCCCTTGCCCGCCCCCTGACGCTGCC
TCAGCTCCTCAGTGCACAGTGCTGCCTCGTCTGAGGGGACAGGAGGATCA
CCCTCTTCGTCGCTTCGGCCAGTGTGTCGGGCTGGGCCCTGACAAGCCAC
CTGAGGAGAGGCTCGGAGCCGGGCCCGGACCCCGGCGATTGCCGCCCGCT
TCTCTCTAGTCTCACGAGGGGTTTCCCGCCTCGCACCCCCACCTCTGGAC
TTGCCTTTCCTTCTCTTCTCCGCGTGTGGAGGGAGCCAGCGCTTAGGCCG
GAGCGAGCCTGGGGGCCGCCCGCCGTGAAGACATCGCGGGGACCGATTCA
CCATG

5) IL-8. IL-8 is a member of the CXC chemokine family. IL-8 is a chemokine produced by macrophages, immune and epithelial cells and is an important mediator of immune reaction in the innate immune system (reviewed in Waugh and Wilson, 2008; Clin Cancer Res 14; 6735). While neutrophil granulocytes are the primary target cells of IL-8, there is a relative wide range of cells (endothelial cells, macrophages, mast cells, and keratinocytes) respond to IL-8. IL-8, also known as neutrophil chemotactic factor, has two primary functions. It induces chemotaxis in target cells, primarily neutrophils but also other granulocytes, causing them to migrate toward the site of infection. IL-8 also induces phagocytosis once they have arrived. IL-8 is also known to be a potent promoter of angiogenesis. In target cells, IL-8 induces a series of physiological responses required for migration and phagocytosis, such as increase of intracellular Ca2+, exocytosis (e.g. histamine release), and respiratory burst.

IL-8 can be secreted by any cells with toll-like receptors that are involved in the innate immune response. Generally, macrophages see the antigen first, and thus are first to release IL-8 to recruit other cells. Both monomer and homodimer forms of IL-8 have been reported to be potent inducers of the chemokines CXCR1 and CXCR2. The homodimer is more potent, but methylation of Leu25 can block activity of the dimers. IL-8 is believed to play a role in the pathogenesis of bronchiolitis, a common respiratory tract disease caused by viral infection. IL-8 is implicated in gingivitis, psoriasis and increased oxidant stress thereby enhancing the recruitment of inflammatory cells to the site of local inflammation.

Protein: IL-8 Gene: IL-8 (Homo sapiens, chromosome 4, 74606223-74609433 [NCBI Reference Sequence: NC_{—}000004.11]; start site location: 74606376; strand: positive)

Gene Identification

| | |
|---|---|
| GeneID | 3576 |
| HGNC | 6025 |
| HPRD | 00909 |
| MIM | 146930 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 1314 | IL8-1 | ACGTCCCATTCGGCTCCTGAGCCA | 2868 |
| 1331 | IL8-3 | GACGTTGACGAAGTCTATCACCCAA | 2939 |
| 1341 | | ACGGAGTATGACGAAAGTTTTC | 257 |
| 1342 | | GAGCGAGACTCCCGTCTAAA | 3259 |

Target Shift Sequences

| | | Relative |
|---|---|---|
| | | upstream |
| Sequence | | location to |
| ID No: | Sequence (5′-3′) | gene start site |
| 1314 | ACGTCCCATTCGGCTCCTGAGCCA | 2868 |
| 1315 | CGTCCCATTCGGCTCCTGAG | 2869 |
| 1316 | GTCCCATTCGGCTCCTGAGC | 2870 |
| 1317 | TCCCATTCGGCTCCTGAGCC | 2871 |
| 1318 | CCCATTCGGCTCCTGAGCCA | 2872 |
| 1319 | CCATTCGGCTCCTGAGCCAT | 2873 |
| 1320 | CATTCGGCTCCTGAGCCATA | 2874 |
| 1321 | ATTCGGCTCCTGAGCCATAA | 2875 |
| 1322 | TTCGGCTCCTGAGCCATAAG | 2876 |
| 1323 | TCGGCTCCTGAGCCATAAGA | 2877 |
| 1324 | CGGCTCCTGAGCCATAAGAA | 2878 |
| 1325 | TACGTCCCATTCGGCTCCTG | 2867 |
| 1326 | TTACGTCCCATTCGGCTCCT | 2866 |
| 1327 | TTTACGTCCCATTCGGCTCC | 2865 |
| 1328 | ATTTACGTCCCATTCGGCTC | 2864 |
| 1329 | TATTTACGTCCCATTCGGCT | 2863 |
| 1330 | TTATTTACGTCCCATTCGGC | 2862 |
| 1331 | GACGTTGACGAAGTCTATCACCCAA | 2939 |
| 1332 | ACGTTGACGAAGTCTATCAC | 2940 |
| 1333 | CGTTGACGAAGTCTATCACC | 2941 |
| 1334 | GTTGACGAAGTCTATCACCC | 2942 |
| 1335 | TTGACGAAGTCTATCACCCA | 2943 |
| 1336 | TGACGAAGTCTATCACCCAA | 2944 |
| 1337 | GACGAAGTCTATCACCCAAG | 2945 |
| 1338 | ACGAAGTCTATCACCCAAGA | 2946 |
| 1339 | CGAAGTCTATCACCCAAGAA | 2947 |
| 1340 | AGACGTTGACGAAGTCTATC | 2938 |
| 1341 | ACGGAGTATGACGAAAGTTTTC | 257 |
| 1342 | GAGCGAGACTCCCGTCTAAA | 3259 |
| 1343 | AGCGAGACTCCCGTCTAAAA | 3260 |
| 1344 | GCGAGACTCCCGTCTAAAAA | 3261 |
| 1345 | CGAGACTCCCGTCTAAAAAA | 3262 |
| 1346 | GAGACTCCCGTCTAAAAAAG | 3263 |
| 1347 | AGAGCGAGACTCCCGTCTAA | 3258 |
| 1348 | AAGAGCGAGACTCCCGTCTA | 3257 |
| 1349 | AAAGAGCGAGACTCCCGTCT | 3256 |
| 1350 | GAAAGAGCGAGACTCCCGTC | 3255 |
| 1351 | TGAAAGAGCGAGACTCCCGT | 3254 |
| 1352 | GTGAAAGAGCGAGACTCCCG | 3253 |
| 1353 | GGTGAAAGAGCGAGACTCCC | 3252 |

Hot Zones (Relative upstream location to gene start site)
1-300
2650-3300
4800-5000

### Examples

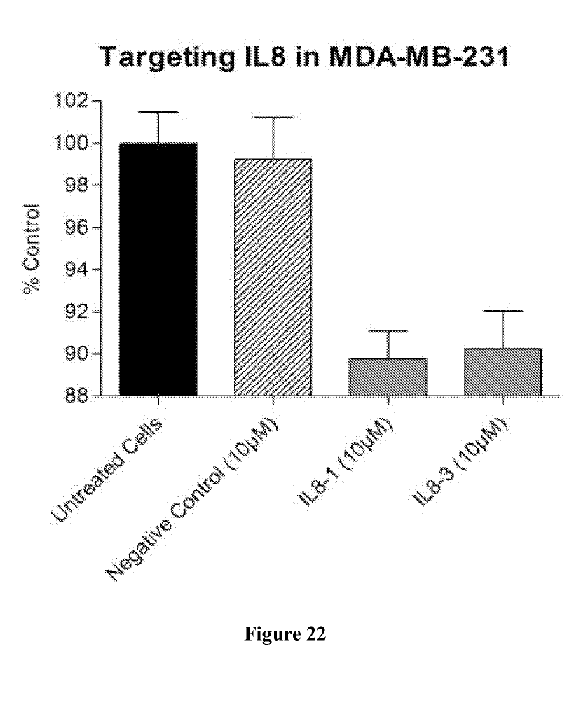

In FIG. 22, IL8-1 (41) and IL8-3 (42), both at 10 μM, demonstrated statistically significant (P<0.05) inhibition compared to the untreated control values in MDA-MB-231 (human breast cell line). The negative control did not produce a statistically significant difference compared to the untreated control. The IL-8 sequences IL8-1 (41) and IL8-3 (42) fit the independent and dependent DNAi motif claims.

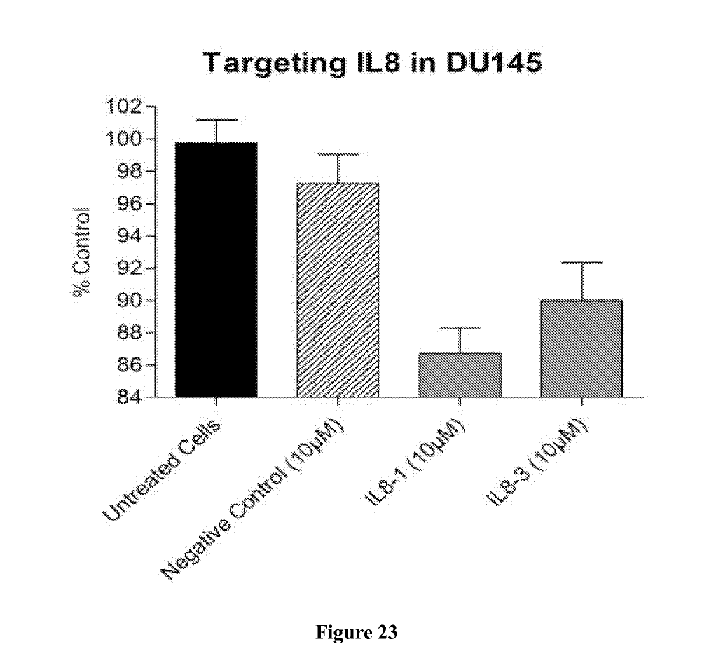

In FIG. 23, IL8-1 (41) and IL8-3 (42), both at 10 μM, demonstrated statistically significant (P<0.05) inhibition compared to the untreated control values in DU145 (human prostate cell line). The negative control did not produce a statistically significant difference compared to the untreated control. The IL-8 sequences IL8-1 (41) and IL8-3 (42) fit the independent and dependent DNAi motif claims.

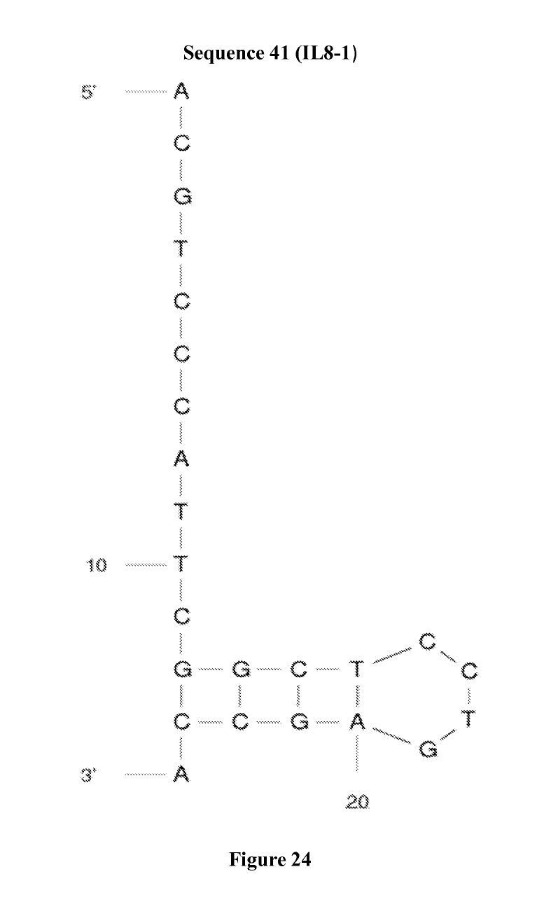

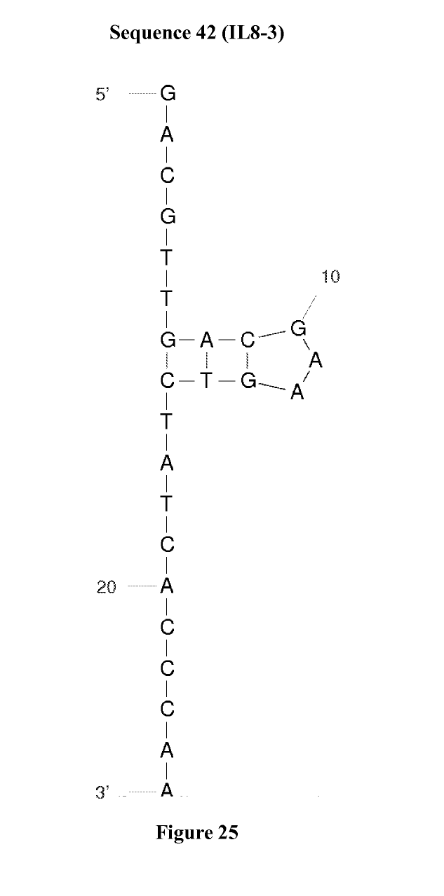

The secondary structures for IL8-1 and IL8-3 are shown in FIGS. 24 and 25. Sequence 41 (IL8-1) is shown in FIG. 24 and Sequence 42 (IL8-3) is shown in FIG. 25.

Genetic Code (5′ Upstream Region)

(SEQ ID NO: 11954)

GGCATTAAAAAAGAAAGCTTATATAGTGGAAGAAAATAAAGCATCTAGAC
ATAAGCTTTAAGAGATCTATTGTGTTAATACAGCTTTACTTTTTGAGTGG
TAAGCTTTTAAAAAGAAATGTGGTGCTCTAACTCCAGGAAAAGATAAGGG
TGACTGAAGTGATAGTCTAGAGGAAAAAGATGCAGACATTTACTGAGTAC
CTCCAATGTGCCAGGTGCCATTCTGGGCATTTTCATTATGTTTCCTCATT
TAATTCTCATGGTGATCCTTTGGAACTGTGTTATTCTCATTTTTACAGAT
GAGGTAACTGAGAGACAGTCAGATTAAAGAACTGCCTATGATTGTTTGGC
TAATAATAAGTGGAGGGGTGAGGCTTGAAGGCAGGTTTGTCTTATTCCAA
CACCCATACATACCCTTAAATTTAAGTTATTCTGACTTGTGTTGCTCAAA
TCCAATGTGTTCAGCTGTTTGCTTCTCCAATTACCAAGATTTTTCTTTAA
AAGGTAGGACACTTTTGGCAACACGAACCAACTTTGCTCAGTATTGTTAT
AAACTGTTAACTGGAGACATTTGAATTTGGAGATGGAACTGAAATGGTCT
TGCGGTACTAGAGAAGATCAAGTTATCACATAAACAAAGTACAGAGCTGA
GAACATATTTTAAATCTTTCCACTAACTCTGACTTTTATTGACTAAAATT
TTAGTGGGCAGTATGTTTATGTTTATGACTCTTAACATTAACAACATCGT
AAGTCAAACTCACTAATATATGTTAAGCATTCTGTTTATGATTCTTTTAA
CCTAGAGGATTGTTGAGCTGGGACTAATTTCCTCAAATGGGAAAAAAACC
CAGGTGAGAGCTGAGACTGCTCCTGAGACTGAGAAAGGCAGCTCTGACGG
GATCTCAGATTTTAGCAGCAGGAGTTGAACAATGGGCATAGAATCAGCTT
GCCCAAGATCTCCTGATTAATAAACCATGGAACAAGATTTAAACCCAAGT
TCATTTCATTTCAAAGCTCATACCACATTTTGCCCACCATATTTTGCTTT
GTTATATGACTACAACTTAGTTCAGGCTTACAAAAAAGTCCTAATTCTAA
AATTCCTATGGCGTGGGTGGGAGGGGATTTAGATGATTTTGCATAGGCAA
GAAACACCCAGTTTCATGGAGTTTGATGGAAGAGTTATGTACTAATATGG
GAAAAGTAGAGGCCATCTTTGTCTTTGTTCTTTCTTTTTTAGACGGGAGT
CTCGCTCTTTCACCCAGGCTGGAGTGCAGTGGCGCTATCTCGGCTCACTG
CAAGCTCCGCCTCCTGGGTTCACGCCATTCTCCTGCCTCAGCCTCCTGAG
TAGCTGGGACTACAGGCGCCCGCCACCGCTCCCGGCTAATTTTTTATATT
TTCAGTAGAGACGGGGTTTCACCGTGTTAGCTAAGATGGTTTGGATCTCC
TGAACTCGTGATCCGCCCGCCTCGGACTCCCAAAGTGCTGGGATTACAGG
CTTGAGCCACCGCGCCCGGCCGTCTTTGTTCTTTCTTGAACTCTTCCTTT
TCTTGGGTGATAGACTTCGTCAACGTCTAATGAGGATATCTAGGTGCTAG
TCTCTGCTCATCAAATGATTCTTATGGCTCAGGAGCCGAATGGGACGTAA
ATAAACAGTTAAGTCTCATGAACTCACTTTGCATTCATCTCTAGAAGATG
ACAAAACATTTGTATTTATGTGTAGCGTGGCACTTTAGTTAAACTTTGTA
CCCCACTTTGCTCTATTTTAAAGCAGAATATCCTTAAAAAGGATACTTAG
TCCTGCTTTTTTTTTTCCGCCTAAGCCCATTTAGTCCTTCTACTCATTAT
GCAAGGACTCAAATGGTTATCTTTACAGAAGTGAGACAAGATAGAATCAA
TGCTCTTGTAGTCACTTCATCTTTGTCCATTCCCACTTCTGATGGAGAGG
GTTCTAGGACATAATGCACTGAAGGTTACATTGTGAGAGATGAACAACAT
TTGCAAAAGAGGTCTTTTTGCCTTGGAAAGGCTTCATTCTTAAAAAAAAA
TGTGAGCATCAAGGTTAAGTAGACCTCATTAGCTCAAACTTTAAGGATGA
TATCAGGATAAAGTTGGGCCCATGAGAAGAGAATGAGAGGGAGATATAGT
GACATGAAAATAAGGAGGAAAACGAGGTGTCTATGTAAGTTGGGCTCACC
ATAAATACAAAGGCAACCGTTAGGGAAAAGCAAAGAAGTCTTTGCACATC
CTCAGAACTCTGAATGTCTTAGTGATGCTGTATGAGTGAGTCTTAATGAT
AGTGAACTGAATCAGTCAAGCCAGGTTGTGTCCATATGAGAATGTGTCTT
TGCTAAACATGCCAACATCACTGAAGCAAAGAAACTTGGAGTTTTCTTTA
AGATATAGGTCTTTTTTACCTATCCGGCCCAAGCTTTCTCTTCTTGTCAC
TCCATGCACTGTGTTCCGTATGCTAAATAGTTTGAGAAACCCAAATGGGC
CATGTTCGCCTACATTTCATTGTCCTGTACTTCCTGTCCTGTACTAGCAA
AGCAGTCCCATTGGTCTTTCTTCTCCTCATTAACAATAAAGGTAACACTT
TTGATGTTGTTTCTTCAGAAAACCTTCATTCATCAAAACTGCCTCAAAGA
TCATGTTTGTTTGATTCCAGAACTTCCTGTAATTACCTGTTATTGTAACA
CTCATCACTGTATTTTACTTACTTGTGTAACTAATTTTCCATATTCTGCA
CTAGACAACAAAGTCCTTTAAGTCAGGTACTATATCTATTTACATAGCAT
TCACATCTCCTACAATAAGGGACATTAGCAGATAAACAACACATATTAAA
TGAATAATGAAGTTTCTGAAATACTACAGTTGAAAACTATAGGAGCTACA
TTATATAGAATAAACATTTACTTTGCTATAGAATTCAGTGTAACCCAGGC
ATTATTTTATCCTCAAGTCTTAGGTTGGTTGGAGAAAGATAACAAAAAGA
AACATGATTGTGCAGAAACAGACAAACCTTTTTGGAAAGCATTTGAAAAT
GGCATTCCCCCTCCACAGTGTGTTCACAGTGTGGGCAAATTCACTGCTCT
GTCGTACTTTCTGAAAATGAAGAACTGTTACACCAAGGTGAATTATTTAT
AAATTATGTACTTGCCCAGAAGCGAACAGACTTTTACTATCATAAGAACC
CTTCCTTGGTGCTCTTTATCTACAGAATCCAAGACCTTTCAAGAAAGGTC
TTGGATTCTTTTCTTCAGGACACTAGGACATAAAGCCACCTTTTTATGAT
TTGTTGAAATTTCTCACTCCATCCCTTTTGCTAGTGATCATGGGTCCTCA
GAGGTCAGACTTGGTGTCCTTGGATAAAGAGCATGAAGCAACAGTGGCTG
AACCAGAGTTGGAACCCAGATGCTCTTTCCACTAAGCATACAACTTTCCA
TTAGATAACACCTCCCTCCCACCCCAACCAAGCAGCTCCAGTGCACCACT
TTCTGGAGCATAAACATACCTTAACTTTACAACTTGAGTGGCCTTGAATA
CTGTTCCTATCTGGAATGTGCTGTTCTCTTTCATCTTCCTCTATTGAAGC
CCTCCTATTCCTCAATGCCTTGCTCCAACTGCCTTTGGAAGATTCTGCTC
TTATGCCTCCACTGGAATTAATGTCTTAGTACCACTTGTCTATTCTGCTA
TATAGTCAGTCCTTACATTGCTTTCTTCTTCTGATAGACCAAACTCTTTA
AGGACAAGTACCTAGTCTTATCTATTTCTAGATCCCCCACATTACTCAGA
AAGTTACTCCATAAATGTTTGTGGAACTGATTTCTATGTGAAGCACATGT
GCCCCTTCACTCTGTTAACATGCATTAGAAAACTAAATCTTTTGAAAAGT
TGTAGTATGCCCCCTAAGAGCAGTAACAGTTCCTAGAAACTCTCTAAAAT
GCTTAGAAAAAGATTTATTTTAAATTACCTCCCCAATAAAATGATTGGCT
GGCTTATCTTCACCATCATGATAGCATCTGTAATTAACTGAAAAAAAATA
ATTATGCCATTAAAAGAAAATCATCCATGATCTTGTTCTAACACCTGCCA
CTCTAGTACTATATCTGTCACATGGTACTATGATAAAGTTATCTAGAAAT
AAAAAAGCATACAATTGATAATTCACCAAATTGTGGAGCTTCAGTATTTT
AAATGTATATTAAAATTAAATTATTTTAAAGATCAAAGAAAACTTTCGTC
ATACTCCGTATTTGATAAGGAACAAATAGGAAGTGTGATGACTCAGGTTT
GCCCTGAGGGGATGGGCCATCAGTTGCAAATCGTGGAATTTCCTCTGACA
TAATGAAAAGATGAGGGTGCATAAGTTCTCTAGTAGGGTGATGATATAAA
AAGCCACCGGAGCACTCCATAAGGCACAAACTTTCAGAGACAGCAGAGCA
CACAAGCTTCTAGGACAAGAGCCAGGAAGAAACCACCGGAAGGAACCATC
TCACTGTGTGTAAACATG

6) KRAS or GTPase KRas also known as V-Ki-ras2 Kirsten rat sarcoma viral oncogene homolog and KRAS, is a protein that in humans is encoded by the KRAS gene (McGrath et al. Nature 1983; 304 (5926): 501-6, Popescu et al., Somat. Cell Mol. Genet. 1985; 11 (2): 149-55) and is usually tethered to cell membranes because by its C-terminal isoprenyl group. The protein product of the normal KRAS gene performs an essential function in normal tissue signaling. A single amino acid substitution resulting from a particular single nucleotide substitution in genomic DNA, is responsible for the activating mutation. Once on, it recruits and activates C-RAF and PI3Kinase necessary for to propagate growth factor and other receptor signals. The transformed protein that results is implicated in various malignancies, including leukemias, lung adenocarcinoma, mucinous adenoma, ductal carcinoma of the pancreas and colorectal carcinoma (Kranenburg, Biochim. Biophys. Acta 2005; 1756 (2): 81-2; Burmer and Loeb, Proc. Natl. Acad. Sci. U.S.A. 86 (7): 2403-7, Tam et al, Clin. Cancer Res. 12 (5): 1647-53, Almoguera et al, Cell 53 (4): 549-54). Several germline KRAS mutations have been found to be associated with Noonan syndrome (Gelb and Tartaglia, Human Molecular Genetics, 2006; 15 (2): R220-226).

Protein: KRAS Gene: KRAS (Homo sapiens, chromosome 12, 25358180-25403854 [NCBI Reference Sequence: NC_{—}000012.11]; start site location: 25398318; strand: negative)

Gene Identification

| | |
|---|---|
| GeneID | 3845 |
| HGNC | 6407 |
| HPRD | 01817 |
| MIM | 190070 |

Targeted Sequences

| | | | Relative upstream |
|---|---|---|---|
| Sequence | Design | | location to gene start |
| ID No: | ID | Sequence (5′-3′) | site |
| 1354 | KR1 | CCCGGAGCGGGACCGGACCGCGG | 5923 |
| 1435 | KR2 | GCCGGACCCACGCGGCGGCCCGCC | 5856 |
| 1516 | KR0525 | AGTCTCCCCTTCCCGGAGACT | 10265 |
| 1535 | | GCCGGGCCGGCTGGAGAGCGGGTC | 5803 |
| 1538 | | TCGCCCCTCCTCCGAGACTTTC | 6626 |
| 1584 | | GCACCCCGCCACCCTCAGGGTCGGC | 6029 |
| 1633 | | GAGCCGCCGCCACCTTCGCCGCCGC | 5475 |
| 1697 | | CGGCATAGTTCCCCGCCTTAC | 2002 |
| 1730 | KR16 | CGGCCCGAGCCTCCGTGACGAGTGC | 146348 |
| 1767 | KR17 | CTGGGAGGGGATCCCTCACCGAGAG | 3328 |

Target Shift Sequences

| | | Relative |
|---|---|---|
| | | upstream |
| Sequence | | location to |
| ID No: | Sequence (5′-3′) | gene start site |
| 1354 | CCCGGAGCGGGACCGGACCGCGG | 5923 |
| 1355 | CCGGAGCGGGACCGGACCGC | 5924 |
| 1356 | CGGAGCGGGACCGGACCGCG | 5925 |
| 1357 | ACCCGGAGCGGGACCGGACC | 5922 |
| 1358 | GACCCGGAGCGGGACCGGAC | 5921 |
| 1359 | TGACCCGGAGCGGGACCGGA | 5920 |
| 1360 | CTGACCCGGAGCGGGACCGG | 5919 |
| 1361 | TCTGACCCGGAGCGGGACCG | 5918 |
| 1362 | TTCTGACCCGGAGCGGGACC | 5917 |
| 1363 | ATTCTGACCCGGAGCGGGAC | 5916 |
| 1364 | AATTCTGACCCGGAGCGGGA | 5915 |
| 1365 | CAATTCTGACCCGGAGCGGG | 5914 |
| 1366 | CCAATTCTGACCCGGAGCGG | 5913 |
| 1367 | GCCAATTCTGACCCGGAGCG | 5912 |
| 1368 | CGCCAATTCTGACCCGGAGC | 5911 |
| 1369 | CCGCCAATTCTGACCCGGAG | 5910 |
| 1370 | GCCGCCAATTCTGACCCGGA | 5909 |
| 1371 | AGCCGCCAATTCTGACCCGG | 5908 |
| 1372 | CAGCCGCCAATTCTGACCCG | 5907 |
| 1373 | GCAGCCGCCAATTCTGACCC | 5906 |
| 1374 | CGCAGCCGCCAATTCTGACC | 5905 |
| 1375 | CCGCAGCCGCCAATTCTGAC | 5904 |
| 1376 | CCCGCAGCCGCCAATTCTGA | 5903 |
| 1377 | CCCCGCAGCCGCCAATTCTG | 5902 |
| 1378 | TCCCCGCAGCCGCCAATTCT | 5901 |
| 1379 | GTCCCCGCAGCCGCCAATTC | 5900 |
| 1380 | TGTCCCCGCAGCCGCCAATT | 5899 |
| 1381 | CTGTCCCCGCAGCCGCCAAT | 5898 |
| 1382 | GCTGTCCCCGCAGCCGCCAA | 5897 |
| 1383 | GGCTGTCCCCGCAGCCGCCA | 5896 |
| 1384 | AGGCTGTCCCCGCAGCCGCC | 5895 |
| 1385 | AAGGCTGTCCCCGCAGCCGC | 5894 |
| 1386 | CAAGGCTGTCCCCGCAGCCG | 5893 |
| 1387 | GCAAGGCTGTCCCCGCAGCC | 5892 |
| 1388 | CGCAAGGCTGTCCCCGCAGC | 5891 |
| 1389 | CCGCAAGGCTGTCCCCGCAG | 5890 |
| 1390 | GCCGCAAGGCTGTCCCCGCA | 5889 |
| 1391 | AGCCGCAAGGCTGTCCCCGC | 5888 |
| 1392 | TAGCCGCAAGGCTGTCCCCG | 5887 |
| 1393 | CTAGCCGCAAGGCTGTCCCC | 5886 |
| 1394 | CCTAGCCGCAAGGCTGTCCC | 5885 |
| 1395 | GCCTAGCCGCAAGGCTGTCC | 5884 |
| 1396 | TGCCTAGCCGCAAGGCTGTC | 5883 |
| 1397 | CTGCCTAGCCGCAAGGCTGT | 5882 |
| 1398 | CCTGCCTAGCCGCAAGGCTG | 5881 |
| 1399 | CCCTGCCTAGCCGCAAGGCT | 5880 |
| 1400 | CCCCTGCCTAGCCGCAAGGC | 5879 |
| 1401 | CCCCCTGCCTAGCCGCAAGG | 5878 |
| 1402 | GCCCCCTGCCTAGCCGCAAG | 5877 |
| 1403 | CGCCCCCTGCCTAGCCGCAA | 5876 |
| 1404 | CCGCCCCCTGCCTAGCCGCA | 5875 |
| 1405 | CCCGCCCCCTGCCTAGCCGC | 5874 |
| 1406 | GCCCGCCCCCTGCCTAGCCG | 5873 |
| 1407 | GGCCCGCCCCCTGCCTAGCC | 5872 |
| 1408 | CGGCCCGCCCCCTGCCTAGC | 5871 |
| 1409 | GCGGCCCGCCCCCTGCCTAG | 5870 |
| 1410 | GGCGGCCCGCCCCCTGCCTA | 5869 |
| 1411 | CGGCGGCCCGCCCCCTGCCT | 5868 |
| 1412 | GCGGCGGCCCGCCCCCTGCC | 5867 |
| 1413 | CGCGGCGGCCCGCCCCCTGC | 5866 |
| 1414 | ACGCGGCGGCCCGCCCCCTG | 5865 |
| 1415 | CACGCGGCGGCCCGCCCCCT | 5864 |
| 1416 | CCACGCGGCGGCCCGCCCCC | 5863 |
| 1417 | CCCACGCGGCGGCCCGCCCC | 5862 |
| 1418 | ACCCACGCGGCGGCCCGCCC | 5861 |
| 1419 | GACCCACGCGGCGGCCCGCC | 5860 |
| 1420 | GGACCCACGCGGCGGCCCGC | 5859 |
| 1421 | CGGACCCACGCGGCGGCCCG | 5858 |
| 1422 | CCGGACCCACGCGGCGGCCC | 5857 |
| 1423 | GCCGGACCCACGCGGCGGCC | 5856 |
| 1424 | TGCCGGACCCACGCGGCGGC | 5855 |
| 1425 | CTGCCGGACCCACGCGGCGG | 5854 |
| 1426 | ACTGCCGGACCCACGCGGCG | 5853 |
| 1427 | GACTGCCGGACCCACGCGGC | 5852 |
| 1428 | GGACTGCCGGACCCACGCGG | 5851 |
| 1429 | GGGACTGCCGGACCCACGCG | 5850 |
| 1430 | AGGGACTGCCGGACCCACGC | 5849 |
| 1431 | GAGGGACTGCCGGACCCACG | 5848 |
| 1432 | GGAGGGACTGCCGGACCCAC | 5847 |
| 1433 | AGGAGGGACTGCCGGACCCA | 5846 |
| 1434 | GAGGAGGGACTGCCGGACCC | 5845 |
| 1435 | GCCGGACCCACGCGGCGGCCCGCC | 5856 |
| 1436 | CCGGACCCACGCGGCGGCCC | 5857 |
| 1437 | CGGACCCACGCGGCGGCCCG | 5858 |
| 1438 | GGACCCACGCGGCGGCCCGC | 5859 |
| 1439 | GACCCACGCGGCGGCCCGCC | 5860 |
| 1440 | ACCCACGCGGCGGCCCGCCC | 5861 |
| 1441 | CCCACGCGGCGGCCCGCCCC | 5862 |
| 1442 | CCACGCGGCGGCCCGCCCCC | 5863 |
| 1443 | CACGCGGCGGCCCGCCCCCT | 5864 |
| 1444 | ACGCGGCGGCCCGCCCCCTG | 5865 |
| 1445 | CGCGGCGGCCCGCCCCCTGC | 5866 |
| 1446 | GCGGCGGCCCGCCCCCTGCC | 5867 |
| 1447 | CGGCGGCCCGCCCCCTGCCT | 5868 |
| 1448 | GGCGGCCCGCCCCCTGCCTA | 5869 |
| 1449 | GCGGCCCGCCCCCTGCCTAG | 5870 |
| 1450 | CGGCCCGCCCCCTGCCTAGC | 5871 |
| 1451 | GGCCCGCCCCCTGCCTAGCC | 5872 |
| 1452 | GCCCGCCCCCTGCCTAGCCG | 5873 |
| 1453 | CCCGCCCCCTGCCTAGCCGC | 5874 |
| 1454 | CCGCCCCCTGCCTAGCCGCA | 5875 |
| 1455 | CGCCCCCTGCCTAGCCGCAA | 5876 |
| 1456 | GCCCCCTGCCTAGCCGCAAG | 5877 |
| 1457 | CCCCCTGCCTAGCCGCAAGG | 5878 |
| 1458 | CCCCTGCCTAGCCGCAAGGC | 5879 |
| 1459 | CCCTGCCTAGCCGCAAGGCT | 5880 |
| 1460 | CCTGCCTAGCCGCAAGGCTG | 5881 |
| 1461 | CTGCCTAGCCGCAAGGCTGT | 5882 |
| 1462 | TGCCTAGCCGCAAGGCTGTC | 5883 |
| 1463 | GCCTAGCCGCAAGGCTGTCC | 5884 |
| 1464 | CCTAGCCGCAAGGCTGTCCC | 5885 |
| 1465 | CTAGCCGCAAGGCTGTCCCC | 5886 |
| 1466 | TAGCCGCAAGGCTGTCCCCG | 5887 |
| 1467 | AGCCGCAAGGCTGTCCCCGC | 5888 |
| 1468 | GCCGCAAGGCTGTCCCCGCA | 5889 |
| 1469 | CCGCAAGGCTGTCCCCGCAG | 5890 |
| 1470 | CGCAAGGCTGTCCCCGCAGC | 5891 |
| 1471 | GCAAGGCTGTCCCCGCAGCC | 5892 |
| 1472 | CAAGGCTGTCCCCGCAGCCG | 5893 |
| 1473 | AAGGCTGTCCCCGCAGCCGC | 5894 |
| 1474 | AGGCTGTCCCCGCAGCCGCC | 5895 |
| 1475 | GGCTGTCCCCGCAGCCGCCA | 5896 |
| 1476 | GCTGTCCCCGCAGCCGCCAA | 5897 |
| 1477 | CTGTCCCCGCAGCCGCCAAT | 5898 |
| 1478 | TGTCCCCGCAGCCGCCAATT | 5899 |
| 1479 | GTCCCCGCAGCCGCCAATTC | 5900 |
| 1480 | TCCCCGCAGCCGCCAATTCT | 5901 |
| 1481 | CCCCGCAGCCGCCAATTCTG | 5902 |
| 1482 | CCCGCAGCCGCCAATTCTGA | 5903 |
| 1483 | CCGCAGCCGCCAATTCTGAC | 5904 |
| 1484 | CGCAGCCGCCAATTCTGACC | 5905 |
| 1485 | GCAGCCGCCAATTCTGACCC | 5906 |
| 1486 | CAGCCGCCAATTCTGACCCG | 5907 |
| 1487 | AGCCGCCAATTCTGACCCGG | 5908 |
| 1488 | GCCGCCAATTCTGACCCGGA | 5909 |
| 1489 | CCGCCAATTCTGACCCGGAG | 5910 |
| 1490 | CGCCAATTCTGACCCGGAGC | 5911 |
| 1491 | GCCAATTCTGACCCGGAGCG | 5912 |
| 1492 | CCAATTCTGACCCGGAGCGG | 5913 |
| 1493 | CAATTCTGACCCGGAGCGGG | 5914 |
| 1494 | AATTCTGACCCGGAGCGGGA | 5915 |
| 1495 | ATTCTGACCCGGAGCGGGAC | 5916 |
| 1496 | TTCTGACCCGGAGCGGGACC | 5917 |
| 1497 | TCTGACCCGGAGCGGGACCG | 5918 |
| 1498 | CTGACCCGGAGCGGGACCGG | 5919 |
| 1499 | TGACCCGGAGCGGGACCGGA | 5920 |
| 1500 | GACCCGGAGCGGGACCGGAC | 5921 |
| 1501 | ACCCGGAGCGGGACCGGACC | 5922 |
| 1502 | CCCGGAGCGGGACCGGACCG | 5923 |
| 1503 | CCGGAGCGGGACCGGACCGC | 5924 |
| 1504 | CGGAGCGGGACCGGACCGCG | 5925 |
| 1505 | TGCCGGACCCACGCGGCGGC | 5855 |
| 1506 | CTGCCGGACCCACGCGGCGG | 5854 |
| 1507 | ACTGCCGGACCCACGCGGCG | 5853 |
| 1508 | GACTGCCGGACCCACGCGGC | 5852 |
| 1509 | GGACTGCCGGACCCACGCGG | 5851 |
| 1510 | GGGACTGCCGGACCCACGCG | 5850 |
| 1511 | AGGGACTGCCGGACCCACGC | 5849 |
| 1512 | GAGGGACTGCCGGACCCACG | 5848 |
| 1513 | GGAGGGACTGCCGGACCCAC | 5847 |
| 1514 | AGGAGGGACTGCCGGACCCA | 5846 |
| 1515 | GAGGAGGGACTGCCGGACCC | 5845 |
| 1516 | AGTCTCCCCTTCCCGGAGACT | 10265 |
| 1517 | GTCTCCCCTTCCCGGAGACT | 10266 |
| 1518 | TCTCCCCTTCCCGGAGACTT | 10267 |
| 1519 | CTCCCCTTCCCGGAGACTTA | 10268 |
| 1520 | TCCCCTTCCCGGAGACTTAA | 10269 |
| 1521 | CCCCTTCCCGGAGACTTAAT | 10270 |
| 1522 | CCCTTCCCGGAGACTTAATC | 10271 |
| 1523 | CCTTCCCGGAGACTTAATCT | 10272 |
| 1524 | CTTCCCGGAGACTTAATCTT | 10273 |
| 1525 | TTCCCGGAGACTTAATCTTG | 10274 |
| 1526 | TCCCGGAGACTTAATCTTGC | 10275 |
| 1527 | CCCGGAGACTTAATCTTGCT | 10276 |
| 1528 | CCGGAGACTTAATCTTGCTT | 10277 |
| 1529 | CGGAGACTTAATCTTGCTTC | 10278 |
| 1530 | AAGTCTCCCCTTCCCGGAGA | 10264 |
| 1531 | TAAGTCTCCCCTTCCCGGAG | 10263 |
| 1532 | TTAAGTCTCCCCTTCCCGGA | 10262 |
| 1533 | GTTAAGTCTCCCCTTCCCGG | 10261 |
| 1534 | AGTTAAGTCTCCCCTTCCCG | 10260 |
| 1535 | GCCGGGCCGGCTGGAGAGCGGGTC | 5803 |
| 1536 | CCGGGCCGGCTGGAGAGCGG | 5804 |
| 1537 | AGCCGGGCCGGCTGGAGAGC | 5802 |
| 1538 | TCGCCCCTCCTCCGAGACTTTC | 6626 |
| 1539 | CGCCCCTCCTCCGAGACTTT | 6627 |
| 1540 | GCCCCTCCTCCGAGACTTTC | 6628 |
| 1541 | CCCCTCCTCCGAGACTTTCA | 6629 |
| 1542 | CCCTCCTCCGAGACTTTCAG | 6630 |
| 1543 | CCTCCTCCGAGACTTTCAGT | 6631 |
| 1544 | CTCCTCCGAGACTTTCAGTT | 6632 |
| 1545 | TCCTCCGAGACTTTCAGTTC | 6633 |
| 1546 | CCTCCGAGACTTTCAGTTCC | 6634 |
| 1547 | CTCCGAGACTTTCAGTTCCA | 6635 |
| 1548 | TCCGAGACTTTCAGTTCCAT | 6636 |
| 1549 | CCGAGACTTTCAGTTCCATT | 6637 |
| 1550 | CGAGACTTTCAGTTCCATTC | 6638 |
| 1551 | ATCGCCCCTCCTCCGAGACT | 6625 |
| 1552 | GATCGCCCCTCCTCCGAGAC | 6624 |
| 1553 | GGATCGCCCCTCCTCCGAGA | 6623 |
| 1554 | AGGATCGCCCCTCCTCCGAG | 6622 |
| 1555 | TAGGATCGCCCCTCCTCCGA | 6621 |
| 1556 | ATAGGATCGCCCCTCCTCCG | 6620 |
| 1557 | GATAGGATCGCCCCTCCTCC | 6619 |
| 1558 | TGATAGGATCGCCCCTCCTC | 6618 |
| 1559 | CTGATAGGATCGCCCCTCCT | 6617 |
| 1560 | CCTGATAGGATCGCCCCTCC | 6616 |
| 1561 | ACCTGATAGGATCGCCCCTC | 6615 |
| 1562 | TACCTGATAGGATCGCCCCT | 6614 |
| 1563 | GTACCTGATAGGATCGCCCC | 6613 |
| 1564 | TGTACCTGATAGGATCGCCC | 6612 |
| 1565 | CTGTACCTGATAGGATCGCC | 6611 |
| 1566 | CCTGTACCTGATAGGATCGC | 6610 |
| 1567 | GCCTGTACCTGATAGGATCG | 6609 |
| 1568 | CGCCTGTACCTGATAGGATC | 6608 |
| 1569 | GCGCCTGTACCTGATAGGAT | 6607 |
| 1570 | AGCGCCTGTACCTGATAGGA | 6606 |
| 1571 | CAGCGCCTGTACCTGATAGG | 6605 |
| 1572 | GCAGCGCCTGTACCTGATAG | 6604 |
| 1573 | AGCAGCGCCTGTACCTGATA | 6603 |
| 1574 | AAGCAGCGCCTGTACCTGAT | 6602 |
| 1575 | AAAGCAGCGCCTGTACCTGA | 6601 |
| 1576 | AAAAGCAGCGCCTGTACCTG | 6600 |
| 1577 | GAAAAGCAGCGCCTGTACCT | 6599 |
| 1578 | GGAAAAGCAGCGCCTGTACC | 6598 |
| 1579 | TGGAAAAGCAGCGCCTGTAC | 6597 |
| 1580 | CTGGAAAAGCAGCGCCTGTA | 6596 |
| 1581 | GCTGGAAAAGCAGCGCCTGT | 6595 |
| 1582 | GGCTGGAAAAGCAGCGCCTG | 6594 |
| 1583 | GGGCTGGAAAAGCAGCGCCT | 6593 |
| 1584 | GCACCCCGCCACCCTCAGGGTCGGC | 6029 |
| 1585 | CACCCCGCCACCCTCAGGGT | 6030 |
| 1586 | ACCCCGCCACCCTCAGGGTC | 6031 |
| 1587 | CCCCGCCACCCTCAGGGTCG | 6032 |
| 1588 | CCCGCCACCCTCAGGGTCGG | 6033 |
| 1589 | CCGCCACCCTCAGGGTCGGC | 6034 |
| 1590 | CGCCACCCTCAGGGTCGGCC | 6035 |
| 1591 | GCCACCCTCAGGGTCGGCCT | 6036 |
| 1592 | CCACCCTCAGGGTCGGCCTA | 6037 |
| 1593 | CACCCTCAGGGTCGGCCTAT | 6038 |
| 1594 | ACCCTCAGGGTCGGCCTATA | 6039 |
| 1595 | CCCTCAGGGTCGGCCTATAC | 6040 |
| 1596 | CCTCAGGGTCGGCCTATACT | 6041 |
| 1597 | CTCAGGGTCGGCCTATACTG | 6042 |
| 1598 | TCAGGGTCGGCCTATACTGG | 6043 |
| 1599 | CAGGGTCGGCCTATACTGGC | 6044 |
| 1600 | AGGGTCGGCCTATACTGGCG | 6045 |
| 1601 | GGGTCGGCCTATACTGGCGC | 6046 |
| 1602 | GGTCGGCCTATACTGGCGCG | 6047 |
| 1603 | GTCGGCCTATACTGGCGCGC | 6048 |
| 1604 | TCGGCCTATACTGGCGCGCA | 6049 |
| 1605 | CGGCCTATACTGGCGCGCAT | 6050 |
| 1606 | GGCCTATACTGGCGCGCATC | 6051 |
| 1607 | GCCTATACTGGCGCGCATCC | 6052 |
| 1608 | CCTATACTGGCGCGCATCCA | 6053 |
| 1609 | CTATACTGGCGCGCATCCAT | 6054 |
| 1610 | TATACTGGCGCGCATCCATT | 6055 |
| 1611 | ATACTGGCGCGCATCCATTT | 6056 |
| 1612 | TACTGGCGCGCATCCATTTA | 6057 |
| 1613 | ACTGGCGCGCATCCATTTAC | 6058 |
| 1614 | CTGGCGCGCATCCATTTACT | 6059 |
| 1615 | TGGCGCGCATCCATTTACTA | 6060 |
| 1616 | GGCGCGCATCCATTTACTAT | 6061 |
| 1617 | GCGCGCATCCATTTACTATC | 6062 |
| 1618 | CGCGCATCCATTTACTATCA | 6063 |
| 1619 | AGCACCCCGCCACCCTCAGG | 6028 |
| 1620 | GAGCACCCCGCCACCCTCAG | 6027 |
| 1621 | AGAGCACCCCGCCACCCTCA | 6026 |
| 1622 | AAGAGCACCCCGCCACCCTC | 6025 |
| 1623 | GAAGAGCACCCCGCCACCCT | 6024 |
| 1624 | CGAAGAGCACCCCGCCACCC | 6023 |
| 1625 | GCGAAGAGCACCCCGCCACC | 6022 |
| 1626 | TGCGAAGAGCACCCCGCCAC | 6021 |
| 1627 | CTGCGAAGAGCACCCCGCCA | 6020 |
| 1628 | GCTGCGAAGAGCACCCCGCC | 6019 |
| 1629 | AGCTGCGAAGAGCACCCCGC | 6018 |
| 1630 | AAGCTGCGAAGAGCACCCCG | 6017 |
| 1631 | GAAGCTGCGAAGAGCACCCC | 6016 |
| 1632 | AGAAGCTGCGAAGAGCACCC | 6015 |
| 1633 | GAGCCGCCGCCACCTTCGCCGCCGC | 5475 |
| 1634 | AGCCGCCGCCACCTTCGCCG | 5476 |
| 1635 | GCCGCCGCCACCTTCGCCGC | 5477 |
| 1636 | CCGCCGCCACCTTCGCCGCC | 5478 |
| 1637 | CGCCGCCACCTTCGCCGCCG | 5479 |
| 1638 | GCCGCCACCTTCGCCGCCGC | 5480 |
| 1639 | CCGCCACCTTCGCCGCCGCC | 5481 |
| 1640 | CGCCACCTTCGCCGCCGCCA | 5482 |
| 1641 | GCCACCTTCGCCGCCGCCAC | 5483 |
| 1642 | CCACCTTCGCCGCCGCCACT | 5484 |
| 1643 | CACCTTCGCCGCCGCCACTG | 5485 |
| 1644 | ACCTTCGCCGCCGCCACTGC | 5486 |
| 1645 | CCTTCGCCGCCGCCACTGCC | 5487 |
| 1646 | CTTCGCCGCCGCCACTGCCG | 5488 |
| 1647 | TTCGCCGCCGCCACTGCCGC | 5489 |
| 1648 | TCGCCGCCGCCACTGCCGCC | 5490 |
| 1649 | CGCCGCCGCCACTGCCGCCG | 5491 |
| 1650 | GCCGCCGCCACTGCCGCCGC | 5492 |
| 1651 | CCGCCGCCACTGCCGCCGCC | 5493 |
| 1652 | CGCCGCCACTGCCGCCGCCG | 5494 |
| 1653 | GCCGCCACTGCCGCCGCCGC | 5495 |
| 1654 | CCGCCACTGCCGCCGCCGCT | 5496 |
| 1655 | CGCCACTGCCGCCGCCGCTG | 5497 |
| 1656 | GCCACTGCCGCCGCCGCTGC | 5498 |
| 1657 | CCACTGCCGCCGCCGCTGCT | 5499 |
| 1658 | CACTGCCGCCGCCGCTGCTG | 5500 |
| 1659 | ACTGCCGCCGCCGCTGCTGC | 5501 |
| 1660 | CTGCCGCCGCCGCTGCTGCC | 5502 |
| 1661 | TGCCGCCGCCGCTGCTGCCT | 5503 |
| 1662 | GCCGCCGCCGCTGCTGCCTC | 5504 |
| 1663 | CCGCCGCCGCTGCTGCCTCC | 5505 |
| 1664 | CGCCGCCGCTGCTGCCTCCG | 5506 |
| 1665 | GCCGCCGCTGCTGCCTCCGC | 5507 |
| 1666 | CCGCCGCTGCTGCCTCCGCC | 5508 |
| 1667 | CGCCGCTGCTGCCTCCGCCG | 5509 |
| 1668 | GCCGCTGCTGCCTCCGCCGC | 5510 |
| 1669 | CCGCTGCTGCCTCCGCCGCC | 5511 |
| 1670 | CGCTGCTGCCTCCGCCGCCG | 5512 |
| 1671 | GCTGCTGCCTCCGCCGCCGC | 5513 |
| 1672 | CTGCTGCCTCCGCCGCCGCG | 5514 |
| 1673 | TGCTGCCTCCGCCGCCGCGG | 5515 |
| 1674 | GCTGCCTCCGCCGCCGCGGC | 5516 |
| 1675 | CTGCCTCCGCCGCCGCGGCC | 5517 |
| 1676 | CGAGCCGCCGCCACCTTCGC | 5474 |
| 1677 | CCGAGCCGCCGCCACCTTCG | 5473 |
| 1678 | GCCGAGCCGCCGCCACCTTC | 5472 |
| 1679 | GGCCGAGCCGCCGCCACCTT | 5471 |
| 1680 | TGGCCGAGCCGCCGCCACCT | 5470 |
| 1681 | CTGGCCGAGCCGCCGCCACC | 5469 |
| 1682 | ACTGGCCGAGCCGCCGCCAC | 5468 |
| 1683 | TACTGGCCGAGCCGCCGCCA | 5467 |
| 1684 | GTACTGGCCGAGCCGCCGCC | 5466 |
| 1685 | AGTACTGGCCGAGCCGCCGC | 5465 |
| 1686 | GAGTACTGGCCGAGCCGCCG | 5464 |
| 1687 | GGAGTACTGGCCGAGCCGCC | 5463 |
| 1688 | GGGAGTACTGGCCGAGCCGC | 5462 |
| 1689 | CGGGAGTACTGGCCGAGCCG | 5461 |
| 1690 | CCGGGAGTACTGGCCGAGCC | 5460 |
| 1691 | GCCGGGAGTACTGGCCGAGC | 5459 |
| 1692 | GGCCGGGAGTACTGGCCGAG | 5458 |
| 1693 | GGGCCGGGAGTACTGGCCGA | 5457 |
| 1694 | GGGGCCGGGAGTACTGGCCG | 5456 |
| 1695 | GGGGGCCGGGAGTACTGGCC | 5455 |
| 1696 | CGGGGGCCGGGAGTACTGGC | 5454 |
| 1697 | CGGCATAGTTCCCCGCCTTAC | 2002 |
| 1698 | GGCATAGTTCCCCGCCTTAC | 2003 |
| 1699 | GCATAGTTCCCCGCCTTACT | 2004 |
| 1700 | CATAGTTCCCCGCCTTACTC | 2005 |
| 1701 | ATAGTTCCCCGCCTTACTCT | 2006 |
| 1702 | TAGTTCCCCGCCTTACTCTG | 2007 |
| 1703 | AGTTCCCCGCCTTACTCTGC | 2008 |
| 1704 | GTTCCCCGCCTTACTCTGCT | 2009 |
| 1705 | TTCCCCGCCTTACTCTGCTC | 2010 |
| 1706 | TCCCCGCCTTACTCTGCTCT | 2011 |
| 1707 | CCCCGCCTTACTCTGCTCTA | 2012 |
| 1708 | CCCGCCTTACTCTGCTCTAC | 2013 |
| 1709 | CCGCCTTACTCTGCTCTACC | 2014 |
| 1710 | CGCCTTACTCTGCTCTACCT | 2015 |
| 1711 | ACGGCATAGTTCCCCGCCTT | 2001 |
| 1712 | CACGGCATAGTTCCCCGCCT | 2000 |
| 1713 | TCACGGCATAGTTCCCCGCC | 1999 |
| 1714 | GTCACGGCATAGTTCCCCGC | 1998 |
| 1715 | GGTCACGGCATAGTTCCCCG | 1997 |
| 1716 | CGGTCACGGCATAGTTCCCC | 1996 |
| 1717 | ACGGTCACGGCATAGTTCCC | 1995 |
| 1718 | CACGGTCACGGCATAGTTCC | 1994 |
| 1719 | ACACGGTCACGGCATAGTTC | 1993 |
| 1720 | CACACGGTCACGGCATAGTT | 1992 |
| 1721 | ACACACGGTCACGGCATAGT | 1991 |
| 1722 | CACACACGGTCACGGCATAG | 1990 |
| 1723 | TCACACACGGTCACGGCATA | 1989 |
| 1724 | ATCACACACGGTCACGGCAT | 1988 |
| 1725 | TATCACACACGGTCACGGCA | 1987 |
| 1726 | GTATCACACACGGTCACGGC | 1986 |
| 1727 | TGTATCACACACGGTCACGG | 1985 |
| 1728 | TTGTATCACACACGGTCACG | 1984 |
| 1729 | ATTGTATCACACACGGTCAC | 1983 |
| 1730 | CGGCCCGAGCCTCCGTGACGAGTGC | 146348 |
| 1731 | GGCCCGAGCCTCCGTGACGA | 146349 |
| 1732 | GCCCGAGCCTCCGTGACGAG | 146350 |
| 1733 | CCCGAGCCTCCGTGACGAGT | 146351 |
| 1734 | CCGAGCCTCCGTGACGAGTG | 146352 |
| 1735 | CGAGCCTCCGTGACGAGTGC | 146353 |
| 1736 | GAGCCTCCGTGACGAGTGCC | 146354 |
| 1737 | AGCCTCCGTGACGAGTGCCA | 146355 |
| 1738 | GCCTCCGTGACGAGTGCCAC | 146356 |
| 1739 | CCTCCGTGACGAGTGCCACC | 146357 |
| 1740 | CTCCGTGACGAGTGCCACCC | 146358 |
| 1741 | TCCGTGACGAGTGCCACCCC | 146359 |
| 1742 | CCGTGACGAGTGCCACCCCC | 146360 |
| 1743 | CGTGACGAGTGCCACCCCCT | 146361 |
| 1744 | GTGACGAGTGCCACCCCCTG | 146362 |
| 1745 | TGACGAGTGCCACCCCCTGC | 146363 |
| 1746 | GACGAGTGCCACCCCCTGCT | 146364 |
| 1747 | ACGAGTGCCACCCCCTGCTC | 146365 |
| 1748 | CGAGTGCCACCCCCTGCTCC | 146366 |
| 1749 | GCGGCCCGAGCCTCCGTGAC | 146347 |
| 1750 | TGCGGCCCGAGCCTCCGTGA | 146346 |
| 1751 | ATGCGGCCCGAGCCTCCGTG | 146345 |
| 1752 | TATGCGGCCCGAGCCTCCGT | 146344 |
| 1753 | CTATGCGGCCCGAGCCTCCG | 146343 |
| 1754 | CCTATGCGGCCCGAGCCTCC | 146342 |
| 1755 | TCCTATGCGGCCCGAGCCTC | 146341 |
| 1756 | CTCCTATGCGGCCCGAGCCT | 146340 |
| 1757 | GCTCCTATGCGGCCCGAGCC | 146339 |
| 1758 | GGCTCCTATGCGGCCCGAGC | 146338 |
| 1759 | GGGCTCCTATGCGGCCCGAG | 146337 |
| 1760 | TGGGCTCCTATGCGGCCCGA | 146336 |
| 1761 | ATGGGCTCCTATGCGGCCCG | 146335 |
| 1762 | CATGGGCTCCTATGCGGCCC | 146334 |
| 1763 | CCATGGGCTCCTATGCGGCC | 146333 |
| 1764 | TCCATGGGCTCCTATGCGGC | 146332 |
| 1765 | CTCCATGGGCTCCTATGCGG | 146331 |
| 1766 | CCTCCATGGGCTCCTATGCG | 146330 |
| 1767 | CTGGGAGGGGATCCCTCACCGAGAG | 3328 |
| 1768 | TGGGAGGGGATCCCTCACCG | 3329 |
| 1769 | GGGAGGGGATCCCTCACCGA | 3330 |
| 1770 | GGAGGGGATCCCTCACCGAG | 3331 |
| 1771 | GAGGGGATCCCTCACCGAGA | 3332 |
| 1772 | AGGGGATCCCTCACCGAGAG | 3333 |
| 1773 | GGGGATCCCTCACCGAGAGT | 3334 |
| 1774 | GGGATCCCTCACCGAGAGTT | 3335 |
| 1775 | GGATCCCTCACCGAGAGTTA | 3336 |
| 1776 | GATCCCTCACCGAGAGTTAG | 3337 |
| 1777 | ATCCCTCACCGAGAGTTAGA | 3338 |
| 1778 | TCCCTCACCGAGAGTTAGAA | 3339 |
| 1779 | CCCTCACCGAGAGTTAGAAA | 3340 |
| 1780 | CCTCACCGAGAGTTAGAAAA | 3341 |
| 1781 | CTCACCGAGAGTTAGAAAAG | 3342 |
| 1782 | TCACCGAGAGTTAGAAAAGC | 3343 |
| 1783 | CACCGAGAGTTAGAAAAGCT | 3344 |

Hot Zones (Relative upstream location to gene start site)
650-1600
1900-2200
2900-3250
3800-4350
4800-6350
6500-7050

### Examples

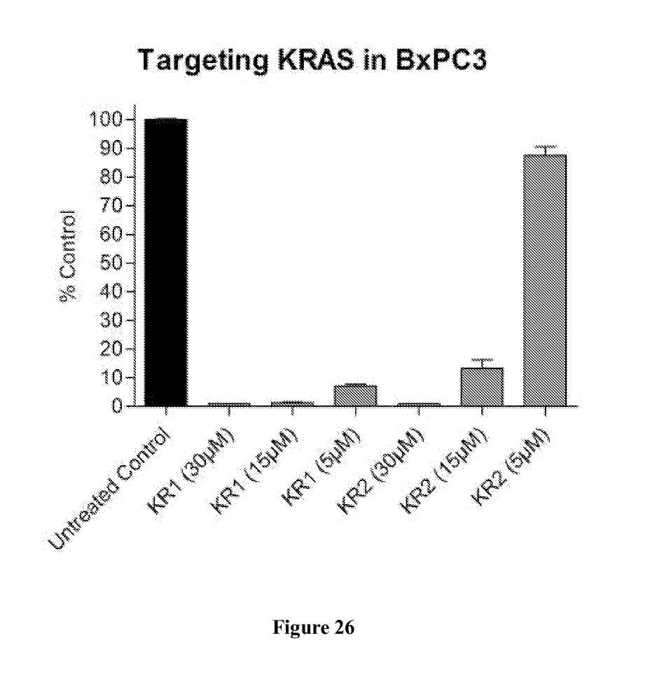

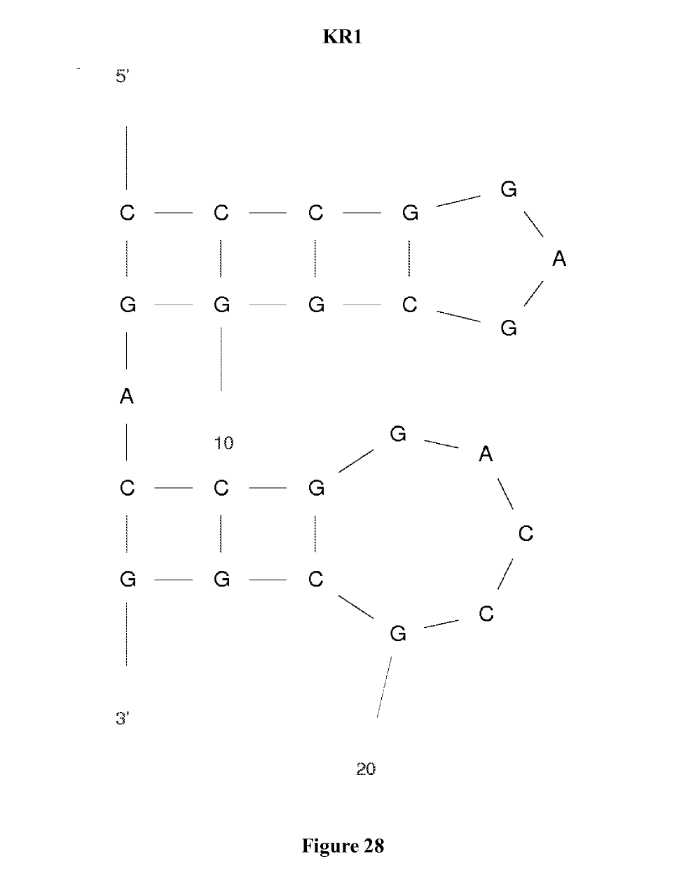

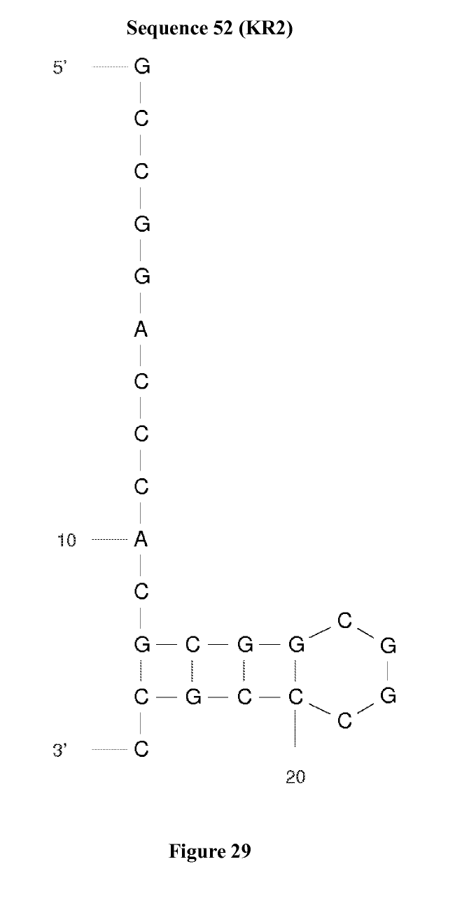

In FIG. 26, Both KR1 (51) and KR2 (52) demonstrated a dose-dependent inhibition response in BxPC3 (human pancreatic cancer cell line), albeit the dose response in KR1 (51) was more subtle. As would be expected, both KR1 (51) and KR2 (52) at 5 μM showed the lowest inhibition while KR1 (51) and KR2 (52) at 30 μM showed the greatest inhibition. Both KR1 (51) and KR2 (52) (FIG. 28 and FIG. 29) fit the independent and dependent DNAi motif claims.

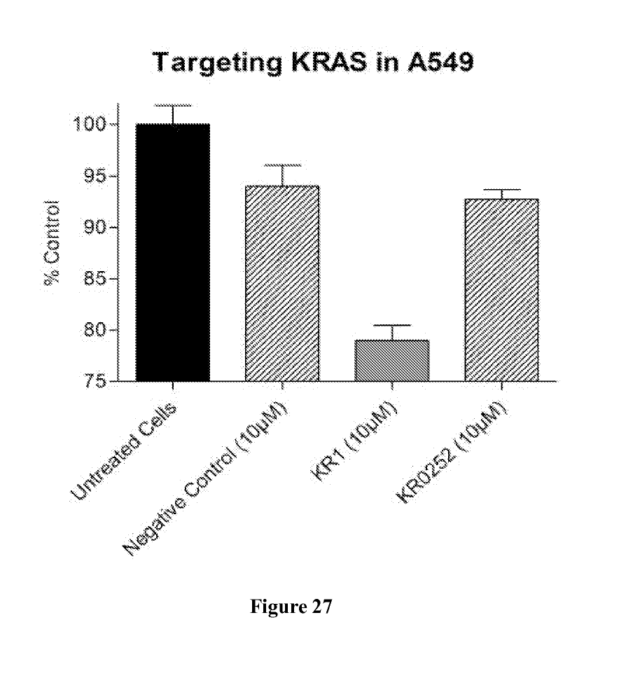

In FIG. 27, A549 (human lung cancer cell line), KR1 shows significant (P<0.05) inhibition at 10 μM. Neither KR0525 nor the negative control demonstrates significant inhibition. Only KR1 (FIG. 28) fits the independent and dependent DNAi motif claims. KR0525's (FIG. 29) lack of inhibition is attributable to: 1) the linear base of the secondary structure either prior to or at the base of the hairpin does not contain a CG pair, 2) its secondary structure does not contain four nucleotides in its base and 3) it is located too far upstream from the KRAS transcription start site (10,265 bases upstream).

The secondary structures for KR1 and KR2 are shown in FIGS. 28 and 29. Sequence 51 (KR1) is shown in FIG. 28 and Sequence 52 (KR2) is shown in FIG. 29.

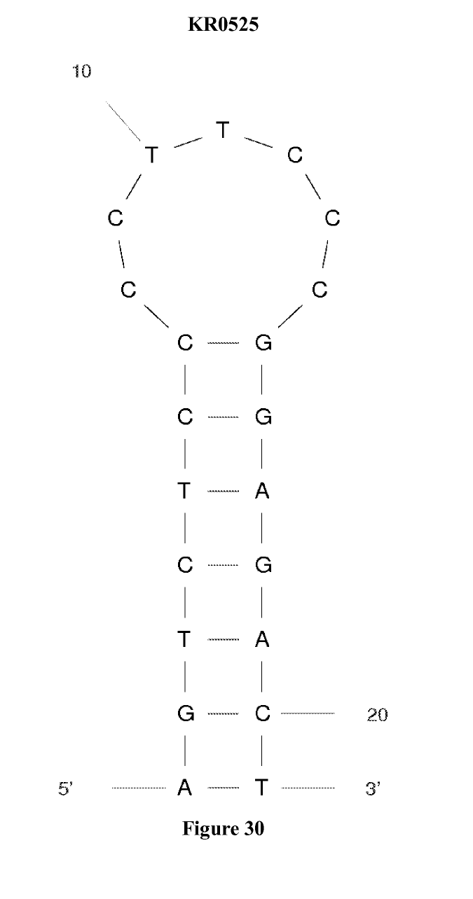

The secondary structure for KR0525 is show in FIG. 30. Sequence 53 (KR0525)—No CG in 5′ linear section of the base either prior to or in the base of the hairpin; does not contain 4 nucleotides in the base; located too far from the start site

Genetic Code (5′ Upstream Region)

(SEQ ID NO: 11955)

TAATCAACAAAGCATTCATGGAGAAAATAGGTCTTATTCTAAATCTTGAA
TGAGAGAGAATTGTAGCAAACAGAAAGACAAAAAGGTGCTGGGTGAGAAA
AGGAGCAGAGACATAAATAAAATATCCAATTTTAAGGGTATAGAGAGGGG
ATTCACTCAAGGAGGGGAGACCATCTATCTGCTTTGAGAAGCTGGGAAAC
AAAGTCATAGGGTCAGGATGGTGCCTGACTATGGATGCTCTCAAAAGCTA
GGCACCAAGGATTTGGACTGGATTCAGCTGGATATAAGAAGTTATTACAG
ACTTGGAAGCAAGATTAAGTCTCCGGGAAGGGGAGACTTAACTGGGACCA
GAGATCATTTTCCCCTATAATTTTAAAGGTACTTATCATCTTTAGGTACT
CATTAGGTACTTAGCTTGTAACTCTTTCCACTGTTCAAATATATACCCAG
TATGCATGTAGCCTATATGGAGCAGGCACAGAGTAAATGTTTGATGATGA
TAAAAGACATGCGGAAGAAAGGTTAATTTGGCAACATCATAAAACTGAAT
TGAGACAAAGAAAGCCAGGAGGTAGGAAAGTCAATGAAGAAGTTATTCCA
GAAATGTAGCTGAGAAGGAAGGAATACAGAAGAGGCAGATATGGGAAAAT
ACTCAGGAAGTATAATTAAAAGGAGCTGTGACTAATTTTAATAAGGACTG
GGTTAAAAATTAAGTTTTCATGTCTAAATGTTCTGGAGGACCATGATGTC
ACTCAGGTAAGATGGAGGAATTGAGAGAGGGAATTCGTTAGAGGGAATAA
CATGGGGAATTTGGCTTTGGACAGGCATTTGCCATGATAACAGAATATTC
ATTTAGAAATGGTCCAGGAAATTGGTTTGATGAGAATGAAGTGCCTGTGA
AGAGAGAGGACTGAAGCTTGTTATAATTTCATTCACTTCAGGAATATTTA
CAGAGGACCCAAATGTGCTAAGAACTATGAAAACATAGAATTAAAAGAAA
TGGGCCTGGAATAATTTACAACCTAGTAAAGCAGTTATGGGAAAACATAT
TTGCAAAAAAGGTATACAAAGTATAATGAAATAAGTGTCCAGTAAGGATA
AAGTGCAGAGTAAGTGAATTAAGCAGCACCCATTCATGTGTTCAAATTCC
TGCCAGAGTCAAAAGGTTGTGCTGAAGTAGAGTCCATGAAAGCATCGTAG
ATGGCTCCTCCTGCTCAAGTTCCCCTGCTCTGCGTCCTGCTACTCTGGCC
ACAACCGTCTGGACCCAGGGTTGACACACAAACAAACACAATAATCTTTT
AGCCAGACATAAAGAAGGCCAGCCACCAATCAGGAAAATTGTGTCCCATA
AAGGCCCTTCCTATTGAACAGTGAATGACAGACATGGCCAGATCTTCTCT
CTTGGAATGCTTTGAATGTTAGTCACAGAGAGTGACCACTAGAAGCACAG
ATAGCAGTAGAAGCTAAGACTACATGAAAAAGCAGTGGACAGATGGTGAT
TTATGAGAATGGCAAAATTACTAGAGTCATAGGCAATGGATACTTGTTAA
TGAAGGGATGAGCAGGGCCCCACAGCCTGTTGCTGGCTCACAAGTGCAGT
TGATTGCTGGACTGAACAGCAGCTCTCCGCCTGATGATAGGGTTTTTTAA
AGTGTCCTTATTGCCTTAAAGTAAATCCTCAGCATTTGCAGTGCTCTGAG
GGTGTCCTAGCATTTTATACCTTTTTTCTAAGAGCCCAGGTAACATAAGG
GTACTCCTGTTGTTCTGGCTTTAATTCTATCTGCAGAAGAGGGTTTCTTG
TGAAAGAAAGGGTCAGTATGGTCTTTTATCTGTACAGCAGATAAAAAGGG
TATGTACGTGCACACCTTTGTACGTGGCTGCCTTCCCAGGACAGTCTGAC
AGTAGAGGGTAGAAACTTCAGTTGTAGCTGAGAGCAGGCCTGGAATCCCC
ATGCTTATACTTTTTATTTCCTCCCCCCTTTCCCATTGTGATCACAGGCT
ACTTCAGTGTGCTTGTCCTTGGAGAGAGCAAGGGAAGGGAGAGCCAGGGA
GACTGTTCAAGGGAGCCACCAGGCTCGAGAAAGAGGAACCCCTGAAGACA
GTAGAAAGTGCAGGTGCCAAGAATTTGAATATCTACATCAGAGTTTCTCA
ATGTGCACACAGTGAACTACCAGTTTAGGATCATTTGATTTGCTAAAAAT
GAAGATTACTGGTCTACCTTAGACCAACTGAATAAAATATCTGGGTGAGG
GGCCTAGGAACTTGCATTTTTGGTAGGCATGGCAGGTGATTCCTAAAGCA
TTTACCCTTGAGACCTCTATGTTAAGGAAAGAAAGGTAATGTTGCAAGGA
GGTGGTGCCGGCTTCTAAGAAAGTACCCAGGACTGAACGGCAGAAAGACC
TGACATACCATATGTATAAATTGCTGTGGAAGTGAAAAGGAAAGAGAAAG
TGTCTGAGGTAAAACTGGAGTGTGGGGTGCGTGGAACAAATGGTTGGATG
CAGATTTGCTTTACGAATCATGAGCCTAGATGATAACTGAGACCATGTGG
ATGGATTAGGTTTCTGCTAATGCCAGAATTTTTATAATCAGCATAAAAGT
GCTATATAAAGCTTTCCCCTCTTCTATATTATAGTCCTTTTAAGATGTAT
GGAACATCAACTATAGGAAGAACATCATATTCACAGCTGTAAGAGGAAAC
AAGAACTTATCATGCACTTGATGTTGTACAAAATAAATCTGTGATTTATG
CTTGAGTGACCACAAAGTAGCATACACATAAGCGCAAATTCATTCATTTA
AGAATTCCTTGTGTCTATTATGTACGAGATAAGTATCTCTGAGCTGCACG
GAATGTGGCTTATCAGAAGGTGACCTAAGTTTCAAAGCAGATTTTGTTAA
GATGAAGACAGAGATTGACAGGAGGTTTAAGACACTCTGTCTAAAGTAAA
GATTTAGAGTCACAGAGTTCATGGATTAGGATTTAGAATCCACAGAGGGT
CCACAGATTCACTCATTCAACATTCCATAAATATTTATTGAATGCCTTTT
TGTGTCAGAGACTGTCTTAGGTGCTGGAAATTTAGCAGTAAATGAAACAG
ACCAAAACCCATGCCCTCATGGAGCTTACATTCTGATGGTAGAGAGACAA
GAAAACAAAATAGATAGTGTATTATTGAAGGTGATGAGAGCTCTGGAGAA
AAAGTAGGAAAAGAGACAGATCTGGGACAAGGGCGAAATTACAGTATCAA
AGATGATCTTTTTAGGGAAGATCTCCTTTTAAAAACACTTTGGAACAAAG
ATTTAAATGAGGTGCCAGAGGGGTAGCAAGTGCATATTCCCTGAGGAAGA
CGCCTGCCTGGCATTTTCAAGGAACAGCCAGTAACCAATGTTTATCTACG
TAAGTAAGGAAGGGAGAACAGTAGGATGAGAGTTCAGAGAAGAGGGTAGG
GGATATCAAATAATTTAAGGCCATGTAGGATTTTTGAGAAGAATTTTGCT
TTTATGTCAAGTGGAATGAGGGCCACTGATGATCTGGGAGTAGAGTGACT
ATGATCCGACATGAAGTATACTCCATTTTTTAACTATGTGAACTTGTGCC
AACGTTTTAACCTCTAAATCTGTTTCGTCATTTGTAAAACGGTAAAAAGT
ATTTTACCTCATAAGGTTGTCGTGATGATTAAATAAGATGATACGATAAG
TGCAAAAGATTTAGCTTGTACTTAACATAGAGTAGGCACATTTTCTCCCC
TTCCCTGTCTTTCACTTTTCTCTTCTGCCCCTTCCACCTGGCGCTAGGAG
GGGGAGACTGGAATAAACCTTGCAGATTACAGCCCGTGTAAGAGTAGAAA
GGAAAGGATGACAGTTGATGTAAAGCCTTGGTTAACAGACATAATAGCTG
GGATTTAAATTCAGCTTTATTGGTGGTTTATGATGTGGACTAGAGGAATG
GAACTGAAAGTCTCGGAGGAGGGGCGATCCTATCAGGTACAGGCGCTGCT
TTTCCAGCCCTCAATCCTCAAGACTCTCCCAAGATACATTTCTAGGTAGT
TTATCAACACAGACTCCGGGTATGCTAGCATGTTTAATTGCCCCATTGTT
TAATGTCTTAACTCCACGAACTTTAACTGATTAATCTGTCTTCTAATTAA
TGTTTGAATGACTCTCCTCAGGTCTAAACTACCAAGGCCATCTCTACTTA
AAAACAGTTGTCTTTTGTTTGTGATTTCAGGGGCCCTGGGTATAAGCGAA
GTCCCTGTTTAGAGACCTTGTGATGGGTTCAAAATATCAAGAAAGATAGC
AAAATATCACAAGCCTCCTGACCCGAGAAGATTAGCGTTGAAAGGGTCTG
TCGTGTTTGTTTGGGCCTGGGGCTAAATTCCCAGCCCAAGTGCTGAGGCT
GATAATAATCGGGGCGGCGATCAGACAGCCCCGGTGTGGGAAATCGTCCG
CCCGGTCTCCCTAAGTCCCCGAAGTCGCCTCCCACTTTTGGTGACTGCTT
GTTTATTTACATGCAGTCAATGATAGTAAATGGATGCGCGCCAGTATAGG
CCGACCCTGAGGGTGGCGGGGTGCTCTTCGCAGCTTCTCTGTGGAGACCG
GTCAGCGGGGCGGCGTGGCCGCTCGCGGCGTCTCCCTGGTGGCATCCGCA
CAGCCCGCCGCGGTCCGGTCCCGCTCCGGGTCAGAATTGGCGGCTGCGGG
GACAGCCTTGCGGCTAGGCAGGGGGCGGGCCGCCGCGTGGGTCCGGCAGT
CCCTCCTCCCGCCAAGGCGCCGCCCAGACCCGCTCTCCAGCCGGCCCGGC
TCGCCACCCTAGACCGCCCCAGCCACCCCTTCCTCCGCCGGCCCGGCCCC
CGCTCCTCCCCCGCCGGCCCGGCCCGGCCCCCTCCTTCTCCCCGCCGGCG
CTCGCTGCCTCCCCCTCTTCCCTCTTCCCACACCGCCCTCAGCCGCTCCC
TCTCGTACGCCCGTCTGAAGAAGAATCGAGCGCGGAACGCATCGATAGCT
CTGCCCTCTGCGGCCGCCCGGCCCCGAACTCATCGGTGTGCTCGGAGCTC
GATTTTCCTAGGCGGCGGCCGCGGCGGCGGAGGCAGCAGCGGCGGCGGCA
GTGGCGGCGGCGAAGGTGGCGGCGGCTCGGCCAGTACTCCCGGCCCCCGC
CATTTCGGACTGGGAGCGAGCGCGGCGCAGGCACTGAAGGCGGCGGCGGG
GCCAGAGGCTCAGCGGCTCCCAGGTGCGGGAGAGAGGTACGGAGCGGACC
ACCCCTCCTGGGCCCCTGCCCGGGTCCCGACCCTCTTTGCCGGCGCCGGG
CGGGGCCGGCGGCGAGTGAATGAATTAGGGGTCCCCGGAGGGGCGGGTGG
GGGGCGCGGGCGCGGGGTCGGGGCGGGCTGGGTGAGAGGGGTCTGCAGGG
GGGAGGCGCGCGGACGCGGCGGCGCGGGGAGTGAGGAATGGGCGGTGCGG
GGCTGAGGAGGGTGAGGCTGGAGGCGGTCGCCGCTGGTGCTGCTTCCTGG
ACGGGGAACCCCTTCCTTCCTCCTCCCCGAGAGCCGCGGCTGGAGGCTTC
TGGGGAGAAACTCGGGCCGGGCCGGCTGCCCCTCGGAGCGGTGGGGTGCG
GTGGAGGTTACTCCCGCGGCGCCCCGGCCTCCCCTCCCCCTCTCCCCGCT
CCCGCACCTCTTGCCTCCCTTTCCAGCACTCGGCTGCCTCGGTCCAGCCT
TCCCTGCTGCATTTGGCATCTCTAGGACGAAGGTATAAACTTCTCCCTCG
AGCGCAGGCTGGACGGATAGTGGTCCTTTTCCGTGTGTAGGGGATGTGTG
AGTAAGAGGGGAGGTCACGTTTTGGAAGAGCATAGGAAAGTGCTTAGAGA
CCACTGTTTGAGGTTATTGTGTTTGGAAAAAAATGCATCTGCCTCCGAGT
TCCTGAATGCTCCCCTCCCCCATGTATGGGCTGTGACATTGCTGTGGCCA
CAAAGGAGGAGGTGGAGGTAGAGATGGTGGAAGAACAGGTGGCCAACACC
CTACACGTAGAGCCTGTGACCTACAGTGAAAAGGAAAAAGTTAATCCCAG
ATGGTCTGTTTTGCTTGGTCAAGTTAAACCCGAAGAAAACCCGCAGAGCA
GAAGCAAGGCTTTTTCCTTGCTAGTTGAGTGTAGACAGCAATAGCAAAAA
TAGTACTTGAAGTTTAATTTACCTGTTCTTGTCCTTTCCCCTATTTCTTA
TGTATTACCCTCATCCCCTCGTCTCTTTTATACTACCCTCATTTTGCAGA
TGTGTTCTACATCTCAAGAGTTATTACAGTACTCCAAAACAGCACTTACA
TGATTTTTTAAACTTACAGAGGAATTGTAGCAATCCACCAGCTAACCGCC
TGAAATAGACTTAAACATGTGCATCTCCTTTTTTTTTTTTTTTTTGAGAC
ACAGTCTCGCTCTGTTGCCCAGGCTGGAGTGCAATGGCGCGGTATCGGCT
CACTGAAACCTCCGCCTCCTGGGTTCAAGCAATTCTCCTGCCTCAGCCTC
CCGAGTAGCTGGGACTAGTAGGTGCACGCCACCATGCCCAGCTAATTTTT
GTATTTTTAGTAGAGACAGAGTTTCATCATGTTGGTCAGGATGGTCTCCA
TCTGCTCTGTTGCCCAGGCTGGAGTGCAGTGGCGCCGTCTCGGCTCACTG
CAACCTCTGCCTCCTGCATTCAAGCAATTCTCCTGCCTCAGCCTCCCGAA
TAACTGGGATTACAGGTGTCTGCTGCCATGCCCGGCTAATTTTTTGTATT
TTTAGTAGAGACGGGGGTTTCACCATGTTGGTCAGGCTGGTCTAGAACTC
CTGACCTCGTGATCTGCCCGCCTCGGCCTCCCACAGTGGCATGTGCATCT
TATAGCTGAAGTCTAAGCCTTCTTAAATCTTGAGATCCATCAAAACAGAC
AGGTTTTCTAATTGTTATACAATGTATATGTTATGTTTATAATAGAAATC
ATTTTACAAATAAGTTATAAATGGGAAAGGTCTATTTGTAATTATCAGCT
CAGAATTAACCATAAAACTGGTGTCACTGAAGTGACTGAGGTCCAAAATG
CTGACTCTGCATGTTATAGACTACAGATATCAAATATGGTTGCTAACAAT
AGTTTACTTTGAGACTGTAGCCATCCACAGTATATTTGCTTTTAAGAGAT
GGTAGATGGTAATTCAGTTTTATGAAAAATAAAAATGAATTTTCTTCCAT
TACAAAATTGTTGGATTCGAGTCCAGTCCACTCCTTACTAGCTTTTCTAA
CTCTCGGTGAGGGATCCCCTCCCAGCCCATGATCTTCATTTGGTAAGACT
CCTTTGGAACCCAGTTCTCTCTAGTGGATTTAAATGTGATTTGGTTTTAA
AAATCTCATTCAAGGAATTTTTTTTTTTTCTGGAAACAACCACCGCATAA
ACAAGTAAACCGGAAGATACATGTGGCTCTGAATTCATATATATACACAA
ACTCTAATCCAATGTCTGTCCACAGTATTTCCTAGGCTAGTAAACTTTTT
GGCCTTAACGACCCCTCTACCCTCTTTGTTTTTTTGAGAGAGAGAGTCTC
ACTCTGTCACCCAGGCCGGAATGCAGTGGCGCGATCTCGGCCCGCTACTA
CCTCCGACTCTCAGGCTCAAGCGATTCTCCCGCCTCAGCTTCCCGAGTAG
CCGGGATTACAGGCTCCCGCCACCGGGCTAATTGTATTTTTAGATACGGG
ATTTCACCATGTTGGCCAGGCTGGTCTCGACCTCCTGACCTCAGGTGATC
CGCCCGCCTAAGCCTCCCAAAGTGCTGGGATTACAGGCCACCACACCCGG
CCTACACTCTTAAAAATTATCGAAGGGGCCGGGCACATTGGCTCTTATCT
GTAATCCCAGCACTTTGGGAGACTGAGGCGGGAGGATCGCTTGAGGCCAG
GAGTTGGAGACCAGCGTACTCAACATAGTGAGACCTTGTTATAAAGAAAA
AAAAAATCCAGGATTAAAAAAAATCTTTGATTTGTTTGGGATTTATTAAT
ATTTACCGTATTGGAAATTAAAACAATTTTTTAAAATGTATTCATTTAAA
AATAATAAGCCCATTACTTGGTAACATGAATAAAATATTTTATGAAAAAT
AACTATTTTCCAAAACAAAACCAAAACTTAGAAAAGTGGTATTGTTTCAC
ACTTCAGTAAATCTCTTTAATGATGTGGCTTAATAGAAGATATGGATTCT
TATATCTGCATCTGCATTCAATCTATTATGATCACACATCTGGAAAACTT
GTGAAAGAATGGGAGTTAAAAGGGTAAAGGACATCTTAATGTTATTATGA
AAACAGTTTTGACCTCTTGCACACCAGAAAAGTCTTAGTAACCTGAGGGG
TTCCTAGACCACATTTTGAGAACTGTTTTAGGCTATGCAAACTGGTTGGG
GGGAGGTTGGGGTAGGCAGAGAGCTAGAAGATACATTTTAGTGTAATTCT
CCTCATCTATTCCTAATTGCTTTGGCCTACATTTGAAATAAAGCGTGGAG
GCAAACGGGATAAGATACATGTTTGTAGTGGTTGTTAACTTCACCCTAGA
CAAGCAGCCAATAAGTCTAGGTAGAGCAGAGTAAGGCGGGGAACTATGCC
GTGACCGTGTGTGATACAATTTTTCTAGCCTGTGGTGCTTTTTGCGGCAG
GGCTTAGGAGTAAGGTTAGTATGTTATCATTTGGGAAACCAAATTATTAT
TTTGGGTCTTCAGTCAATTATGATGCTGTGTATATTTAGTGTTTATCTAC
AATATATGCACATTCATTAATTTGGAGCTACTCATCCTATAATAAATAGT
TGTGCATTTACTCCCATTTTTTTCTGCATTTCTCTCCTTATTTATAATTA
TGTGTTACATGAGGGAAAGGAGGTGAAATTAAACATTCATATTATTTCAA
AAAATTTGAAACAACTAACTAAAAAATATGTTTTATTTTCTGTATGGTGT
TTGTTATACAATCTGTCAATATTCATGCACCTCTTGGGAGACAGTGTATG
AAAAGCAAAGAGTAACAGTCACATGGATTACTGATTACTGAGATATATTC
ACTTGCATCTTTTTTTTTTTTTGAGACGGAGTGGCTCTGTCGCCCAGGCT
GGAGTGCAGTGGCGTGATCTCGGCTCACTGCAAGCTCCGCCTCCTGGGTT
CACGCCATTCTTCTGCCTCAGCCTCCCAAGTAGCTGGGACTACAGGCGCC
CGCCACCACGCCCGGCTAATTTTTTTATATTTTTAGTAGAGACGGGGTTT
CACCGGGTTAGCCAGGATGGTCTTGATCTCCTGACCTCGTGATCCACCCT
CCTCGGCCTCCCAAAGTGCTAGGATTATAGGCGTGAGCCACCGTGCCCGG
CTCACTTGCATCTCTTAACAGCTGTTTTCTTACTAAAAACAGTGTTTATC
TCTAATCTTTTTGTTTGTTTGTTTGTTTTGAGATGGAGTCTTACTCCGTC
ACCCAATCTGGAGTGCAGTGGCGTGATCTGGGCTCACTGCAACCTCTGCC
TCCCGGGTTCAAGTGATTCTCCTTCCTCAGCCTCCCCAGTAGCTAGGACT
ACAGGAGAGCGCCACCACGCCTGATTAATTTTTGTATTTTTAGTAGAGAG
AGGGTTTCACCATATTGGCCAGGCTGGTCTTGAACTCCTGGCCTCAGGTG
ATCCACCCGCCTTGGCCTCTGAAAGTGCTGGGATTACAGGCATGAGCCGC
CGCACCCGGCTTTCTAATCTTTATCTTTTTTTGTGCAGCGGTGATACAGG
ATTATGTATTGTACTGAACAGTTAATTCGGAGTTCTCTTGGTTTTTAGCT
TTATTTTCCCCAGAGATTTTTTTTTTTTTTTTTTTTTTTGAGACGGAGTC
TTGCTCTATCGCCAGGCTGGAGTGCAGTGGCGCCATCTCGGCTCATTGCA
ACCTCGGACTCCTATTTTCCCCAGAGATATTTCACACATTAAAATGTCGT
CAAATATTGTTCTTCTTTGCCTCAGTGTTTAAATTTTTATTTCCCCATGA
CACAATCCAGCTTTATTTGACACTCATTCTCTCAACTCTCATCTGATTCT
TACTGTTAATATTTATCCAAGAGAACTACTGCCATGATGCTTTAAAAGTT
TTTCTGTAGCTGTTGCATATTGACTTCTAACACTTAGAGGTGGGGGTCCA
CTAGGAAAACTGTAACAATAAGAGTGGAGATAGCTGTCAGCAACTTTTGT
GAGGGTGTGCTACAGGGTGTAGAGCACTGTGAAGTCTCTACATGAGTGAA
GTCATGATATGATCCTTTGAGAGCCTTTAGCCGCCGCAGAACAGCAGTCT
GGCTATTTAGATAGAACAACTTGATTTTAAGATAAAAGAACTGTCTATGT
AGCATTTATGCATTTTTCTTAAGCGTCGATGGAGGAGTTTGTAAATGAAG
TACAGTTCATTACGATACACGTCTGCAGTCAACTGGAATTTTCATGATTG
AATTTTGTAAGGTATTTTGAAATAATTTTTCATATAAAGGTGAGTTTGTA
TTAAAAGGTACTGGTGGAGTATTTGATAGTGTATTAACCTTATGTGTGAC
ATGTTCTAATATAGTCACATTTTCATTATTTTTATTATAAGGCCTGCTGA
AAATG

7) MTTP. Microsomal triglyceride transfer protein is an an essential chaperone for the biosynthesis/lipoprotein assembly of apolipoprotein B (apoB)-containing triglyceride-rich lipoproteins Inhibition of MTTP prevents the assembly of apo B-containing lipoproteins by inhibiting chylomicrons and VLDL synthesis. As a result, decreases in plasma levels of LDL-C are observed (Shoulders et al., Hum Mol Genet 2 (12): 2109-16). Patients carry mutations in the MTTP gene exhibit abetalipoproteinemia resulting from the loss of its lipid transfer activity.

MTTP is also recognized to play a role in the biosynthesis of CD1, glycolipid presenting molecules, as well as in the regulation of cholesterol ester biosynthesis. Recently, MTTP has been implicated in the propagation of hepatitis C virus, where the virus hijacks lipoprotein assembly for its secretion. Therefore, MTTP is a good target to lower plasma lipids and treat disorders characterized by higher production of apoB-containing lipoproteins such as atherosclerosis, metabolic syndrome, familial combined hyperlipidemia, homozygous and heterozygous familial hypercholesterolemia and hypertriglyceridemia (reviewed in Hussain et al. Nutrition & Metabolism 2012, 9:14). MTTP is also recognized to be involved in the immune response against foreign lipid antigens, such that targeting it may also be useful for modulating the inflammatory response during T cell mediated processes such as inflammatory bowel disease, autoimmune hepatitis and asthma (Hussain et al., Curr Opin Lipidol 2008, 19:277-284). Current therapies that inhibit MTTP without increasing hepatic lipids and plasma transaminases are lacking.

Protein: MTTP Gene: MTTP (Homo sapiens, chromosome 4, 100485240-100545154 [NCBI Reference Sequence: NC_{—}000004.11]; start site location: 100496067; strand: positive)

Gene Identification

| | |
|---|---|
| GeneID | 4547 |
| HGNC | 7467 |
| HPRD | 01144 |
| MIM | 157147 |

Targeted Sequences

| | | Relative |
|---|---|---|
| | | upstream |
| Sequence | | location to |
| ID No: | Sequence (5′-3′) | gene start site |
| 1784 | AACCGCCGTAGCCTCCACTGCG | 10855 |
| 1870 | TGGCCGCAGTTCGATGACGTAAGACG | 10828 |

Target Shift Sequences

| | | Relative |
|---|---|---|
| | | upstream |
| Sequence | | location to gene |
| ID No: | Sequence (5′-3′) | start site |
| 1784 | AACCGCCGTAGCCTCCACTGCG | 10855 |
| 1785 | ACCGCCGTAGCCTCCACTGC | 10856 |
| 1786 | CCGCCGTAGCCTCCACTGCG | 10857 |
| 1787 | CGCCGTAGCCTCCACTGCGT | 10858 |
| 1788 | GCCGTAGCCTCCACTGCGTA | 10859 |
| 1789 | CCGTAGCCTCCACTGCGTAA | 10860 |
| 1790 | CGTAGCCTCCACTGCGTAAC | 10861 |
| 1791 | GTAGCCTCCACTGCGTAACT | 10862 |
| 1792 | TAGCCTCCACTGCGTAACTA | 10863 |
| 1793 | AGCCTCCACTGCGTAACTAC | 10864 |
| 1794 | GCCTCCACTGCGTAACTACC | 10865 |
| 1795 | CCTCCACTGCGTAACTACCG | 10866 |
| 1796 | CTCCACTGCGTAACTACCGC | 10867 |
| 1797 | TCCACTGCGTAACTACCGCC | 10868 |
| 1798 | CCACTGCGTAACTACCGCCC | 10869 |
| 1799 | CACTGCGTAACTACCGCCCC | 10870 |
| 1800 | ACTGCGTAACTACCGCCCCT | 10871 |
| 1801 | CTGCGTAACTACCGCCCCTG | 10872 |
| 1802 | TGCGTAACTACCGCCCCTGC | 10873 |
| 1803 | GCGTAACTACCGCCCCTGCC | 10874 |
| 1804 | CGTAACTACCGCCCCTGCCT | 10875 |
| 1805 | GTAACTACCGCCCCTGCCTC | 10876 |
| 1806 | TAACTACCGCCCCTGCCTCT | 10877 |
| 1807 | AACTACCGCCCCTGCCTCTG | 10878 |
| 1808 | ACTACCGCCCCTGCCTCTGG | 10879 |
| 1809 | CTACCGCCCCTGCCTCTGGG | 10880 |
| 1810 | TACCGCCCCTGCCTCTGGGA | 10881 |
| 1811 | ACCGCCCCTGCCTCTGGGAA | 10882 |
| 1812 | CCGCCCCTGCCTCTGGGAAT | 10883 |
| 1813 | CGCCCCTGCCTCTGGGAATT | 10884 |
| 1814 | CAACCGCCGTAGCCTCCACT | 10854 |
| 1815 | GCAACCGCCGTAGCCTCCAC | 10853 |
| 1816 | CGCAACCGCCGTAGCCTCCA | 10852 |
| 1817 | ACGCAACCGCCGTAGCCTCC | 10851 |
| 1818 | GACGCAACCGCCGTAGCCTC | 10850 |
| 1819 | AGACGCAACCGCCGTAGCCT | 10849 |
| 1820 | AAGACGCAACCGCCGTAGCC | 10848 |
| 1821 | TAAGACGCAACCGCCGTAGC | 10847 |
| 1822 | GTAAGACGCAACCGCCGTAG | 10846 |
| 1823 | CGTAAGACGCAACCGCCGTA | 10845 |
| 1824 | ACGTAAGACGCAACCGCCGT | 10844 |
| 1825 | GACGTAAGACGCAACCGCCG | 10843 |
| 1826 | TGACGTAAGACGCAACCGCC | 10842 |
| 1827 | ATGACGTAAGACGCAACCGC | 10841 |
| 1828 | GATGACGTAAGACGCAACCG | 10840 |
| 1829 | CGATGACGTAAGACGCAACC | 10839 |
| 1830 | TCGATGACGTAAGACGCAAC | 10838 |
| 1831 | TTCGATGACGTAAGACGCAA | 10837 |
| 1832 | GTTCGATGACGTAAGACGCA | 10836 |
| 1833 | AGTTCGATGACGTAAGACGC | 10835 |
| 1834 | CAGTTCGATGACGTAAGACG | 10834 |
| 1835 | GCAGTTCGATGACGTAAGAC | 10833 |
| 1836 | CGCAGTTCGATGACGTAAGA | 10832 |
| 1837 | CCGCAGTTCGATGACGTAAG | 10831 |
| 1838 | GCCGCAGTTCGATGACGTAA | 10830 |
| 1839 | GGCCGCAGTTCGATGACGTA | 10829 |
| 1840 | TGGCCGCAGTTCGATGACGT | 10828 |
| 1841 | ATGGCCGCAGTTCGATGACG | 10827 |
| 1842 | AATGGCCGCAGTTCGATGAC | 10826 |
| 1843 | AAATGGCCGCAGTTCGATGA | 10825 |
| 1844 | GAAATGGCCGCAGTTCGATG | 10824 |
| 1845 | CGAAATGGCCGCAGTTCGAT | 10823 |
| 1846 | TCGAAATGGCCGCAGTTCGA | 10822 |
| 1847 | TTCGAAATGGCCGCAGTTCG | 10821 |
| 1848 | GTTCGAAATGGCCGCAGTTC | 10820 |
| 1849 | GGTTCGAAATGGCCGCAGTT | 10819 |
| 1850 | GGGTTCGAAATGGCCGCAGT | 10818 |
| 1851 | CGGGTTCGAAATGGCCGCAG | 10817 |
| 1852 | GCGGGTTCGAAATGGCCGCA | 10816 |
| 1853 | TGCGGGTTCGAAATGGCCGC | 10815 |
| 1854 | TTGCGGGTTCGAAATGGCCG | 10814 |
| 1855 | ATTGCGGGTTCGAAATGGCC | 10813 |
| 1856 | CATTGCGGGTTCGAAATGGC | 10812 |
| 1857 | CCATTGCGGGTTCGAAATGG | 10811 |
| 1858 | TCCATTGCGGGTTCGAAATG | 10810 |
| 1859 | TTCCATTGCGGGTTCGAAAT | 10809 |
| 1860 | CTTCCATTGCGGGTTCGAAA | 10808 |
| 1861 | TCTTCCATTGCGGGTTCGAA | 10807 |
| 1862 | TTCTTCCATTGCGGGTTCGA | 10806 |
| 1863 | TTTCTTCCATTGCGGGTTCG | 10805 |
| 1864 | CTTTCTTCCATTGCGGGTTC | 10804 |
| 1865 | CCTTTCTTCCATTGCGGGTT | 10803 |
| 1866 | CCCTTTCTTCCATTGCGGGT | 10802 |
| 1867 | CCCCTTTCTTCCATTGCGGG | 10801 |
| 1868 | TCCCCTTTCTTCCATTGCGG | 10800 |
| 1869 | CTCCCCTTTCTTCCATTGCG | 10799 |
| 1870 | TGGCCGCAGTTCGATGACGTAAGACG | 10828 |
| 1871 | GGCCGCAGTTCGATGACGTA | 10829 |
| 1872 | GCCGCAGTTCGATGACGTAA | 10830 |
| 1873 | CCGCAGTTCGATGACGTAAG | 10831 |
| 1874 | CGCAGTTCGATGACGTAAGA | 10832 |
| 1875 | GCAGTTCGATGACGTAAGAC | 10833 |
| 1876 | CAGTTCGATGACGTAAGACG | 10834 |
| 1877 | AGTTCGATGACGTAAGACGC | 10835 |
| 1878 | GTTCGATGACGTAAGACGCA | 10836 |
| 1879 | TTCGATGACGTAAGACGCAA | 10837 |
| 1880 | TCGATGACGTAAGACGCAAC | 10838 |
| 1881 | CGATGACGTAAGACGCAACC | 10839 |
| 1882 | GATGACGTAAGACGCAACCG | 10840 |
| 1883 | ATGACGTAAGACGCAACCGC | 10841 |
| 1884 | TGACGTAAGACGCAACCGCC | 10842 |
| 1885 | GACGTAAGACGCAACCGCCG | 10843 |
| 1886 | ACGTAAGACGCAACCGCCGT | 10844 |
| 1887 | CGTAAGACGCAACCGCCGTA | 10845 |
| 1888 | GTAAGACGCAACCGCCGTAG | 10846 |
| 1889 | TAAGACGCAACCGCCGTAGC | 10847 |
| 1890 | AAGACGCAACCGCCGTAGCC | 10848 |
| 1891 | AGACGCAACCGCCGTAGCCT | 10849 |
| 1892 | GACGCAACCGCCGTAGCCTC | 10850 |
| 1893 | ACGCAACCGCCGTAGCCTCC | 10851 |
| 1894 | CGCAACCGCCGTAGCCTCCA | 10852 |
| 1895 | GCAACCGCCGTAGCCTCCAC | 10853 |
| 1896 | CAACCGCCGTAGCCTCCACT | 10854 |
| 1897 | AACCGCCGTAGCCTCCACTG | 10855 |
| 1898 | ACCGCCGTAGCCTCCACTGC | 10856 |
| 1899 | CCGCCGTAGCCTCCACTGCG | 10857 |
| 1900 | CGCCGTAGCCTCCACTGCGT | 10858 |
| 1901 | GCCGTAGCCTCCACTGCGTA | 10859 |
| 1902 | CCGTAGCCTCCACTGCGTAA | 10860 |
| 1903 | CGTAGCCTCCACTGCGTAAC | 10861 |
| 1904 | GTAGCCTCCACTGCGTAACT | 10862 |
| 1905 | TAGCCTCCACTGCGTAACTA | 10863 |
| 1906 | AGCCTCCACTGCGTAACTAC | 10864 |
| 1907 | GCCTCCACTGCGTAACTACC | 10865 |
| 1908 | CCTCCACTGCGTAACTACCG | 10866 |
| 1909 | CTCCACTGCGTAACTACCGC | 10867 |
| 1910 | TCCACTGCGTAACTACCGCC | 10868 |
| 1911 | CCACTGCGTAACTACCGCCC | 10869 |
| 1912 | CACTGCGTAACTACCGCCCC | 10870 |
| 1913 | ACTGCGTAACTACCGCCCCT | 10871 |
| 1914 | CTGCGTAACTACCGCCCCTG | 10872 |
| 1915 | TGCGTAACTACCGCCCCTGC | 10873 |
| 1916 | GCGTAACTACCGCCCCTGCC | 10874 |
| 1917 | CGTAACTACCGCCCCTGCCT | 10875 |
| 1918 | GTAACTACCGCCCCTGCCTC | 10876 |
| 1919 | TAACTACCGCCCCTGCCTCT | 10877 |
| 1920 | AACTACCGCCCCTGCCTCTG | 10878 |
| 1921 | ACTACCGCCCCTGCCTCTGG | 10879 |
| 1922 | CTACCGCCCCTGCCTCTGGG | 10880 |
| 1923 | TACCGCCCCTGCCTCTGGGA | 10881 |
| 1924 | ACCGCCCCTGCCTCTGGGAA | 10882 |
| 1925 | CCGCCCCTGCCTCTGGGAAT | 10883 |
| 1926 | CGCCCCTGCCTCTGGGAATT | 10884 |
| 1927 | ATGGCCGCAGTTCGATGACG | 10827 |
| 1928 | AATGGCCGCAGTTCGATGAC | 10826 |
| 1929 | AAATGGCCGCAGTTCGATGA | 10825 |
| 1930 | GAAATGGCCGCAGTTCGATG | 10824 |
| 1931 | CGAAATGGCCGCAGTTCGAT | 10823 |
| 1932 | TCGAAATGGCCGCAGTTCGA | 10822 |
| 1933 | TTCGAAATGGCCGCAGTTCG | 10821 |
| 1934 | GTTCGAAATGGCCGCAGTTC | 10820 |
| 1935 | GGTTCGAAATGGCCGCAGTT | 10819 |
| 1936 | GGGTTCGAAATGGCCGCAGT | 10818 |
| 1937 | CGGGTTCGAAATGGCCGCAG | 10817 |
| 1938 | GCGGGTTCGAAATGGCCGCA | 10816 |
| 1939 | TGCGGGTTCGAAATGGCCGC | 10815 |
| 1940 | TTGCGGGTTCGAAATGGCCG | 10814 |
| 1941 | ATTGCGGGTTCGAAATGGCC | 10813 |
| 1942 | CATTGCGGGTTCGAAATGGC | 10812 |
| 1943 | CCATTGCGGGTTCGAAATGG | 10811 |
| 1944 | TCCATTGCGGGTTCGAAATG | 10810 |
| 1945 | TTCCATTGCGGGTTCGAAAT | 10809 |
| 1946 | CTTCCATTGCGGGTTCGAAA | 10808 |
| 1947 | TCTTCCATTGCGGGTTCGAA | 10807 |
| 1948 | TTCTTCCATTGCGGGTTCGA | 10806 |
| 1949 | TTTCTTCCATTGCGGGTTCG | 10805 |
| 1950 | CTTTCTTCCATTGCGGGTTC | 10804 |
| 1951 | CCTTTCTTCCATTGCGGGTT | 10803 |
| 1952 | CCCTTTCTTCCATTGCGGGT | 10802 |
| 1953 | CCCCTTTCTTCCATTGCGGG | 10801 |
| 1954 | TCCCCTTTCTTCCATTGCGG | 10800 |
| 1955 | CTCCCCTTTCTTCCATTGCG | 10799 |

Hot Zones (Relative upstream location to gene start site)
10750-10900

### Examples

Genetic Code (5′ Upstream Region)

(SEQ ID NO: 11956)

TCTTGAAAATAATCTGTCCTCTCTATCTAGTTCCTTTAAATATCTTCTCT
CTCTCTCTGATATTCTGCAGTTTAATTATGATGTATCTACTTGTGTGTAT
GTGTGTTTTAAAAATTATCCTGCTTAAGACTTATTGAGCCTCGTGAATCT
GTGGATTGGTATCTGTGATAGGCAGACAATGGCTCCCCAAAGATCTTAAA
TGTCTTAATCTCCAGAACCTGTGATAGTCTAAGTTAAGGTTGTAGATGAA
ATTAAAGTTACCAATCCACAGACCTCAGGGTAAAAAGATTATCCTGGATT
ATTTAGGCAGGCCCAGTATAATCACAAGGATTCATAAAACGGAAGAGGGA
GACAGAAGAGATGGTCAGAGTGATATGAAGTAAAAAGGATTCAGCTTACT
CTTGCTGGATTTGTAAATGCAGGAAGGGACCACGAGTCAAGGAATGCAGG
TAGCTTCTAGAAGCTAGAAAAGGTAAGAAACAAATTCTCCCCTAAAGCCT
CCAGAAAGGGATACACCTGCCAATACTTTCATTTTATCCCTGTGAGACCA
GTGTTAGACTTCTGACCTCCAAGAGTATAAGACAATAAATCTGCTGTTTT
AAGCCACTAAGTTTTGTGGTAATTTGTTATGGTAGCTATGGAGAACTGAT
ACAGTGCCTTTCAATAGTTCTTGGAAATTCTTCAAATATATTCCCCAAAT
ATTGCCTTTGCACCACTCACTCTATCCTCTATATCTCTTGACCTCTCTTT
AACATTTTTTATTTTCTTATTTTGGTAATTATTTAAAACAATTGGCTTCT
TGTTCCACTTCAAATAAATTCATATTTTTATATCTACATATTAAGAATTA
GTTCATAAATGTAATTGTTGTATCGTATATACTTTAAAAGGAAAATTGCA
TTTATACTTGGATATTATTATATTTTAGGTTTTGAAATTTCTTTTTTTAA
ATGTCTGATAAATTTATTCTGATCAAAATTAAAATCTTATTGTATTTACA
GTAGTCTACTAAAAGAGTTTACATCAATTCTCTCCTTTAAATGCTAGCAA
TTCAGTTTTGTGTGGTCAAAGATAATTTAGAATCCTTTTATGGTAACTGA
TATGATACAAAGGATTTTTTTGGTTGTTGTCAAATTTGTATGTGCGTATA
TATGTATAGGGGGTAGAAAATTTGGTTAGTGACTCTATTTTGGAAAAATG
AATGTTCTTTTTGGAGTTTTAGATTCCCAGTGTTTCAAACCAAGTTTGCT
TTTGATAAGGAATTCAGTGAATCTTTATTTTCTCGTAGAGAATTTTTAAA
CAATACCTTTCAAAATATTGTATGTATTCCTATATAATTTTGCCTTGGAA
TAAAAAAAGTCATCACATTAAGAATATTTTTAACTTAAGAAATTTTTTCA
AGATTCTTTCAAAATGCTAACTCCCTCATTGATTTGAAAATACTTTTAGA
GTTTAAACTATTATGACCATGTAGGGATAACTTAAAGTGTCCTTCTAAAT
TTTTTTTTCATTAATGTCATGCTTCTTTCTAAGAACAAAGTGTTTCAATG
TTATAAACAGACTGTTTTTTCACATAGCTTTCATACATCCTGACTTTCTA
TATCTAGTAGGGTAATACATTTCCTTCCAACCTTTAGTGGGGTGAAGTTC
ATTTGTCTTCTAATCTAAGAAAATACGTTAACTCCTGACAACCTCTGACA
CTCAACAGAAACACAACTGTACTTTGGAATTAATCATCCATCTTTATTTC
ATAGTCTCCTTATTTATCAATGCAAATGGAAGTATAGCAAATATTTCACA
GTGTCATGTACTACCTAAGGAATTTCATTGCAATAGCATTGTTTTGAAAT
GTATCATTTGATTTTGATCTAACTATCATAGATCAGTTTTACCCCTGTAT
GTTCAGCCTGAATGTCTAACCACAATTTCACAAAAATCAAGGGCTCTGTT
AGTTTTCATACAAAATTGTGATGACTTATTATGAAGACAGTCCCAATTAA
CAATTGCATGTCATCTGTAAGAAATCAATTTTTTTCTCACTCCCCACCTG
TAATTTTTTTTAAGCAAAGAAGAATACTTGGGTCTAGGTTTCAAGGTTTA
TTTTTCTGTAGTCCTAATATCACTTCAGTAAATTAATCTGAGACTCATTT
TTCTAATATGTCAAATGACTGTTGTAAGGATTATTATAAAAACGTAAGGT
GATTAACAAAGTATGTAATTGTTCCATAGATGGCAGCTCTTGTTTTTTTA
CTGTGCTTTGCTTCTGCCCTGCTTCACATATTATGTAAAATAGCTGGTGA
GTTTTAGGAGGTAGTGCTCACATGTTTGCACAGTGTTTGCTGTGGACGAT
CAACAGTAACAGAAGAGCATACTTCTTCGATACACAGAGTTTACTGAATT
TGGAAAGGCTTTGGCATTCTTATGTCATCTGTAATGAAACAATCTCCAGA
AGTCTTTTCTAAAATGTCCTTGTAAAAAGAAAAAAGTTATGTTTATATTT
TATAAAAGGATGATGTTATTTATAACCAGCAGAAGCAGCCTTATTTGAAC
ATCTTATGTTGAAATTGCTACTTAATACAGTGACTCATAGGAGCTTTCTA
GTGGAAATCAAATGCTCAAATGAAATAGAATTTAGTTTGTTAGGCAATAG
TGATATGTCTTTTATTGGTTGGACTCTGGAAAACACTTGACAACGAATAG
TACTTTACCCGAAGGGCACGTATCATGCACCACATAGCCTAACCACAAAC
ATTAAAGGTCTTGTAACTGTGAGCCTCAAATGAAAATACATAAGGACAGC
TCTCATACAATCAAATACAATACAAACTAGCTTTTAATTTAAATAAATAT
GTAAGTAAAGTTCAAGTGACTATAATGATTTTATATCCCTATGTATGTAT
CACAGAAATTGTGGCAAACTGTAGAAATCTATTCAAATGGAAAGTAACAA
AGCACTTTCACATTGCCTTGTATTCAAAATCCCTACTCTTCATAAAAACT
TATATTTCTTTAACAAAGCTACTTTTCTGTTTAACTCCCGGAAAACTTCG
TATTTATAACTTAAGGGGGTTTCTCCAACCAAACAATTTATTTTTGCTAG
GTACTATAGCTATATTTTTTATACAAAATTTGTGACAGCAAATGAAATTC
TAATCCCAATAGAAGAACAAACAATTTTCATGTTTCGATCTTCATATATA
TAATTCAAGAGGAAATATGCTTAACTTTGTAGATTTTTACATTTTAAATT
GCATTGTGTCTGTATCAAGTCTACTATCTTTTACCTAGATTGTCTGGAAG
ATTTAAGCTCAAGGTTACGGTTTGAGAAAAGGGTTTTGAGAGTGACCAGG
ATAGATTTAAGAATTCATTTTATACTAAAATATGGCCATAAATATTTTTA
AATACATTCAAATAGCCCTTTGCTGGCACATTTTTTCCCTTCTTTGCCAA
AACATTCCCACAGGCGGCCTAAGTCACCTCATTTTATAGGTTTAGTAGGT
TTAGCAGGCTTTATGTGCTCTAGTAGGGTTAGTAGGTTTTGTTCATATCA
GGTCTCTCTCATGGGAGTTTCCAGGGACAAGGATTGCTTCAGTTAGTATG
GCCTTAGCCATACTAGGGTATTTGCTTTAATTCTACAGAAGTTTTCTAAT
TAATATTCTGTAGCAAAAGAACTAAGATCTGGAATTCCCCCTCTTAATCT
CTTCCTAGAAATGAGATTCAGAAAGGACAGGACTGCATCCAGCCTGTTTG
GGAACTCAGACAAATGTGTGTTGTCACAGACACAAATAGAGGTCTACTAT
GAAATAATTGGCTTGCTAGTGTGCTAATGACAGACAATGCTGATTTGCTC
CAACCTCATACAGTTTCACACATAAGGACAATCATCTATGTTTCATGAAA
GTTCTATCTACTTTAACATTATTTTGAAGTGATTGGTGGTGGTATGAATT
AACAGTTTAAATTTAAATCCTAAAATTCAGTGTGAATTTTTTATAATAGC
ATAAAAATTCAAAGATGTCCATACAAGAAAAATTAAAATTTGGTTAGGTT
TAGCAGAGTTTGAGAATCCTTACTACCCTCCCACATAGTATTGTAATGTG
AATATAGGCAGTTACTATTACAGGCATAATGATGATTATGTATTAAGCAG
AAAGAAGTATCACCACCAGTTTTTTTCTTTGAATGCCCCTCAGTACTTCT
GCATTTATAGGATGGTAGACTGGTTTGGTTTAGCTCTCAAAAGTGAAAAC
ATTTAAAGTTTCCTCATTGGGTGAAAAAAATTAAAAAGAGTGAGAGACTG
AAAACTGCAGCCCACCTACGTTTAATCATTAATAGTGAGCCCTTCAGTGA
ACTTAGGTCCTGATTTTGGAGTTTGGAGTCTGACCTTTCCCCAAAGATAA
ACATGATTGTTGCAGGTTCTGAAGAGGGTCACTCCCTCACTGGCTGCCAT
TGAAAGAGTCCACTTCTCAGTGACTCCTAGCTGGGCACTGGATGCAGTTG
AGGATTGCTGGTCAATATG

8) ApoC III. Apolipoprotein C-III is a protein component of very low density lipoprotein (VLDL). APOC3 inhibits lipoprotein lipase and hepatic lipase; it is thought to inhibit hepatic uptake of triglyceride-rich particles (reviewed in Mendevil et al., Arteriosclerosis, Thrombosis and Vascular Biology 30 (2): 239-45). The APOA1, APOC3 and APOA4 genes are closely linked in both rat and human genomes. The A-I and A-IV genes are transcribed from the same strand, while the A-1 and C-III genes are convergently transcribed. An increase in apoC-III levels induces the development of hypertriglyceridemia. Two novel susceptibility haplotypes (specifically, P2-S2-X1 and P1-S2-X1) have been discovered in ApoAI-CIII-AIV gene cluster on chromosome 11q23; these confer approximately threefold higher risk of coronary heart disease in normal as well as non-insulin diabetes mellitus. Apo-CIII delays the catabolism of triglyceride rich particles. Elevations of Apo-CIII found in genetic variation studies may predispose patients to non-alcoholic fatty liver disease.

ISIS-APOCIIIRx is an antisense drug designed to reduce apolipoprotein C-III, or apoC-III, protein production and lower triglycerides. ApoC-III regulates triglyceride metabolism in the blood and is an independent cardiovascular risk factor. People who do not produce apoC-III have lower levels of triglycerides and lower instances of cardiovascular disease. ApoC-III is elevated in patients with dyslipidemia, or an abnormal concentration of lipids in the blood, and is frequently associated with multiple metabolic abnormalities, such as insulin resistance and/or metabolic syndrome. In human population studies, lower levels of apoC-III and triglycerides correlated with a lower rate of cardiovascular events. In certain populations, apoC-III mediates insulin resistance, which can make metabolic syndrome worse.

Protein: ApoC-III Gene: APOC3 (Homo sapiens, chromosome 11, 116700624-116703787 [NCBI Reference Sequence: NC_{—}000011.9]; start site location: 116701299; strand: positive)

Gene Identification

| | |
|---|---|
| GeneID | 345 |
| HGNC | 610 |
| HPRD | 00132 |
| MIM | 107720 |

Targeted Sequences

| | | Relative |
|---|---|---|
| | | upstream |
| | | location |
| Sequence | | to gene |
| ID No: | Sequence (5′-3′) | start site |
| 1956 | GAGTCGGTGGTCCAGGAGGGGCCGC | 1614 |
| 1957 | CTGCGGCTGAGGTGTCATTCGTGACTCAG | 4214 |
| 1992 | GCGGGCGGGTGAGACAGAAGCGCC | 4130 |
| 1993 | CCTCGCGAGCGTGGGTGCACGC | 3985 |
| 2028 | CGATGTCTCCCTCGAGATCACA | 3717 |
| 2054 | GGACGGACGGATATCTGAGGCCAG | 2195 |
| 2062 | CGTCCCCGCCACGTTGAAAGGC | 3954 |
| 2089 | TCTCGGACATGCTCAAATGGTGCAGGCG | 4080 |
| 2108 | CACCGACAGGAGCCAATAGTGCAACG | 4065 |
| 2127 | GTCCGGCAGAGGGACCCATGCTGACG | 4940 |
| 2136 | CGTGAGGCACATGTCCGTGTG | 3511 |
| 2170 | CAGATGCAGCAAGCGGGCGGGAGAG | 798 |
| 2176 | CCACGCTGCTGTCCCGCCAGCCCTGCAG | 848 |
| 2206 | ACCCGCCCCCACCCTGTGTGCCCCCACCC | 1276 |
| | GCCCCCACCCTGTGTGCCCCC | |
| 2225 | CGCTCAGAGCCCGAGGCCTTTG | 1352 |

Target Shift Sequences

| | | Relative |
|---|---|---|
| | | upstream |
| | | location |
| Sequence | | to gene |
| ID No: | Sequence (5′-3′) | start site |
| 1956 | GAGTCGGTGGTCCAGGAGGGGCCGC | 1614 |
| 1957 | CTGCGGCTGAGGTGTCATTCGTGACTCAG | 4214 |
| 1958 | TGCGGCTGAGGTGTCATTCG | 4215 |
| 1959 | GCGGCTGAGGTGTCATTCGT | 4216 |
| 1960 | CGGCTGAGGTGTCATTCGTG | 4217 |
| 1961 | GGCTGAGGTGTCATTCGTGA | 4218 |
| 1962 | GCTGAGGTGTCATTCGTGAC | 4219 |
| 1963 | CTGAGGTGTCATTCGTGACT | 4220 |
| 1964 | TGAGGTGTCATTCGTGACTC | 4221 |
| 1965 | GAGGTGTCATTCGTGACTCA | 4222 |
| 1966 | AGGTGTCATTCGTGACTCAG | 4223 |
| 1967 | GGTGTCATTCGTGACTCAGT | 4224 |
| 1968 | GTGTCATTCGTGACTCAGTC | 4225 |
| 1969 | TGTCATTCGTGACTCAGTCT | 4226 |
| 1970 | GTCATTCGTGACTCAGTCTC | 4227 |
| 1971 | TCATTCGTGACTCAGTCTCC | 4228 |
| 1972 | CATTCGTGACTCAGTCTCCT | 4229 |
| 1973 | ATTCGTGACTCAGTCTCCTC | 4230 |
| 1974 | TTCGTGACTCAGTCTCCTCC | 4231 |
| 1975 | TCGTGACTCAGTCTCCTCCT | 4232 |
| 1976 | CGTGACTCAGTCTCCTCCTC | 4233 |
| 1977 | ACTGCGGCTGAGGTGTCATT | 4213 |
| 1978 | AACTGCGGCTGAGGTGTCAT | 4212 |
| 1979 | AAACTGCGGCTGAGGTGTCA | 4211 |
| 1980 | CAAACTGCGGCTGAGGTGTC | 4210 |
| 1981 | TCAAACTGCGGCTGAGGTGT | 4209 |
| 1982 | GTCAAACTGCGGCTGAGGTG | 4208 |
| 1983 | GGTCAAACTGCGGCTGAGGT | 4207 |
| 1984 | AGGTCAAACTGCGGCTGAGG | 4206 |
| 1985 | GAGGTCAAACTGCGGCTGAG | 4205 |
| 1986 | GGAGGTCAAACTGCGGCTGA | 4204 |
| 1987 | TGGAGGTCAAACTGCGGCTG | 4203 |
| 1988 | CTGGAGGTCAAACTGCGGCT | 4202 |
| 1989 | CCTGGAGGTCAAACTGCGGC | 4201 |
| 1990 | TCCTGGAGGTCAAACTGCGG | 4200 |
| 1991 | GTCCTGGAGGTCAAACTGCG | 4199 |
| 1992 | GCGGGCGGGTGAGACAGAAGCGCC | 4130 |
| 1993 | CCTCGCGAGCGTGGGTGCACGC | 3985 |
| 1994 | CTCGCGAGCGTGGGTGCACG | 3986 |
| 1995 | TCGCGAGCGTGGGTGCACGC | 3987 |
| 1996 | CGCGAGCGTGGGTGCACGCA | 3988 |
| 1997 | GCGAGCGTGGGTGCACGCAT | 3989 |
| 1998 | CGAGCGTGGGTGCACGCATG | 3990 |
| 1999 | GAGCGTGGGTGCACGCATGG | 3991 |
| 2000 | AGCGTGGGTGCACGCATGGG | 3992 |
| 2001 | GCGTGGGTGCACGCATGGGC | 3993 |
| 2002 | CGTGGGTGCACGCATGGGCT | 3994 |
| 2003 | GTGGGTGCACGCATGGGCTG | 3995 |
| 2004 | TGGGTGCACGCATGGGCTGT | 3996 |
| 2005 | GGGTGCACGCATGGGCTGTG | 3997 |
| 2006 | GGTGCACGCATGGGCTGTGC | 3998 |
| 2007 | GTGCACGCATGGGCTGTGCC | 3999 |
| 2008 | TGCACGCATGGGCTGTGCCA | 4000 |
| 2009 | GCACGCATGGGCTGTGCCAG | 4001 |
| 2010 | CACGCATGGGCTGTGCCAGT | 4002 |
| 2011 | ACGCATGGGCTGTGCCAGTC | 4003 |
| 2012 | CGCATGGGCTGTGCCAGTCC | 4004 |
| 2013 | CCCTCGCGAGCGTGGGTGCA | 3984 |
| 2014 | CCCCTCGCGAGCGTGGGTGC | 3983 |
| 2015 | TCCCCTCGCGAGCGTGGGTG | 3982 |
| 2016 | GTCCCCTCGCGAGCGTGGGT | 3981 |
| 2017 | GGTCCCCTCGCGAGCGTGGG | 3980 |
| 2018 | AGGTCCCCTCGCGAGCGTGG | 3979 |
| 2019 | CAGGTCCCCTCGCGAGCGTG | 3978 |
| 2020 | GCAGGTCCCCTCGCGAGCGT | 3977 |
| 2021 | AGCAGGTCCCCTCGCGAGCG | 3976 |
| 2022 | CAGCAGGTCCCCTCGCGAGC | 3975 |
| 2023 | GCAGCAGGTCCCCTCGCGAG | 3974 |
| 2024 | GGCAGCAGGTCCCCTCGCGA | 3973 |
| 2025 | AGGCAGCAGGTCCCCTCGCG | 3972 |
| 2026 | AAGGCAGCAGGTCCCCTCGC | 3971 |
| 2027 | AAAGGCAGCAGGTCCCCTCG | 3970 |
| 2028 | CGATGTCTCCCTCGAGATCACA | 3717 |
| 2029 | GATGTCTCCCTCGAGATCAC | 3718 |
| 2030 | ATGTCTCCCTCGAGATCACA | 3719 |
| 2031 | TGTCTCCCTCGAGATCACAC | 3720 |
| 2032 | GTCTCCCTCGAGATCACACA | 3721 |
| 2033 | TCTCCCTCGAGATCACACAG | 3722 |
| 2034 | CTCCCTCGAGATCACACAGG | 3723 |
| 2035 | TCCCTCGAGATCACACAGGC | 3724 |
| 2036 | CCCTCGAGATCACACAGGCC | 3725 |
| 2037 | CCTCGAGATCACACAGGCCT | 3726 |
| 2038 | CTCGAGATCACACAGGCCTT | 3727 |
| 2039 | TCGAGATCACACAGGCCTTT | 3728 |
| 2040 | CGAGATCACACAGGCCTTTC | 3729 |
| 2041 | GCGATGTCTCCCTCGAGATC | 3716 |
| 2042 | GGCGATGTCTCCCTCGAGAT | 3715 |
| 2043 | AGGCGATGTCTCCCTCGAGA | 3714 |
| 2044 | GAGGCGATGTCTCCCTCGAG | 3713 |
| 2045 | AGAGGCGATGTCTCCCTCGA | 3712 |
| 2046 | GAGAGGCGATGTCTCCCTCG | 3711 |
| 2047 | GGAGAGGCGATGTCTCCCTC | 3710 |
| 2048 | TGGAGAGGCGATGTCTCCCT | 3709 |
| 2049 | TTGGAGAGGCGATGTCTCCC | 3708 |
| 2050 | CTTGGAGAGGCGATGTCTCC | 3707 |
| 2051 | GCTTGGAGAGGCGATGTCTC | 3706 |
| 2052 | GGCTTGGAGAGGCGATGTCT | 3705 |
| 2053 | AGGCTTGGAGAGGCGATGTC | 3704 |
| 2054 | GGACGGACGGATATCTGAGGCCAG | 2195 |
| 2055 | GACGGACGGATATCTGAGGC | 2196 |
| 2056 | ACGGACGGATATCTGAGGCC | 2197 |
| 2057 | CGGACGGATATCTGAGGCCA | 2198 |
| 2058 | GGACGGATATCTGAGGCCAG | 2199 |
| 2059 | GACGGATATCTGAGGCCAGG | 2200 |
| 2060 | ACGGATATCTGAGGCCAGGA | 2201 |
| 2061 | CGGATATCTGAGGCCAGGAG | 2202 |
| 2062 | CGTCCCCGCCACGTTGAAAGGC | 3954 |
| 2063 | GTCCCCGCCACGTTGAAAGG | 3955 |
| 2064 | TCCCCGCCACGTTGAAAGGC | 3956 |
| 2065 | CCCCGCCACGTTGAAAGGCA | 3957 |
| 2066 | CCCGCCACGTTGAAAGGCAG | 3958 |
| 2067 | CCGCCACGTTGAAAGGCAGC | 3959 |
| 2068 | CGCCACGTTGAAAGGCAGCA | 3960 |
| 2069 | GCCACGTTGAAAGGCAGCAG | 3961 |
| 2070 | CCACGTTGAAAGGCAGCAGG | 3962 |
| 2071 | CACGTTGAAAGGCAGCAGGT | 3963 |
| 2072 | ACGTTGAAAGGCAGCAGGTC | 3964 |
| 2073 | CGTTGAAAGGCAGCAGGTCC | 3965 |
| 2074 | ACGTCCCCGCCACGTTGAAA | 3953 |
| 2075 | CACGTCCCCGCCACGTTGAA | 3952 |
| 2076 | TCACGTCCCCGCCACGTTGA | 3951 |
| 2077 | GTCACGTCCCCGCCACGTTG | 3950 |
| 2078 | GGTCACGTCCCCGCCACGTT | 3949 |
| 2079 | AGGTCACGTCCCCGCCACGT | 3948 |
| 2080 | CAGGTCACGTCCCCGCCACG | 3947 |
| 2081 | ACAGGTCACGTCCCCGCCAC | 3946 |
| 2082 | AACAGGTCACGTCCCCGCCA | 3945 |
| 2083 | TAACAGGTCACGTCCCCGCC | 3944 |
| 2084 | TTAACAGGTCACGTCCCCGC | 3943 |
| 2085 | ATTAACAGGTCACGTCCCCG | 3942 |
| 2086 | CATTAACAGGTCACGTCCCC | 3941 |
| 2087 | TCATTAACAGGTCACGTCCC | 3940 |
| 2088 | TTCATTAACAGGTCACGTCC | 3939 |
| 2089 | TCTCGGACATGCTCAAATGGTGCAGGCG | 4080 |
| 2090 | CTCGGACATGCTCAAATGGT | 4081 |
| 2091 | TCGGACATGCTCAAATGGTG | 4082 |
| 2092 | CGGACATGCTCAAATGGTGC | 4083 |
| 2093 | CTCTCGGACATGCTCAAATG | 4079 |
| 2094 | GCTCTCGGACATGCTCAAAT | 4078 |
| 2095 | TGCTCTCGGACATGCTCAAA | 4077 |
| 2096 | ATGCTCTCGGACATGCTCAA | 4076 |
| 2097 | GATGCTCTCGGACATGCTCA | 4075 |
| 2098 | GGATGCTCTCGGACATGCTC | 4074 |
| 2099 | TGGATGCTCTCGGACATGCT | 4073 |
| 2100 | GTGGATGCTCTCGGACATGC | 4072 |
| 2101 | GGTGGATGCTCTCGGACATG | 4071 |
| 2102 | TGGTGGATGCTCTCGGACAT | 4070 |
| 2103 | CTGGTGGATGCTCTCGGACA | 4069 |
| 2104 | TCTGGTGGATGCTCTCGGAC | 4068 |
| 2105 | CTCTGGTGGATGCTCTCGGA | 4067 |
| 2106 | ACTCTGGTGGATGCTCTCGG | 4066 |
| 2107 | CACTCTGGTGGATGCTCTCG | 4065 |
| 2108 | CACCGACAGGAGCCAATAGTGCAACG | 4876 |
| 2109 | ACCGACAGGAGCCAATAGTG | 4877 |
| 2110 | CCGACAGGAGCCAATAGTGC | 4878 |
| 2111 | CGACAGGAGCCAATAGTGCA | 4879 |
| 2112 | TCACCGACAGGAGCCAATAG | 4875 |
| 2113 | CTCACCGACAGGAGCCAATA | 4874 |
| 2114 | ACTCACCGACAGGAGCCAAT | 4873 |
| 2115 | CACTCACCGACAGGAGCCAA | 4872 |
| 2116 | GCACTCACCGACAGGAGCCA | 4871 |
| 2117 | TGCACTCACCGACAGGAGCC | 4870 |
| 2118 | CTGCACTCACCGACAGGAGC | 4869 |
| 2119 | ACTGCACTCACCGACAGGAG | 4868 |
| 2120 | CACTGCACTCACCGACAGGA | 4867 |
| 2121 | GCACTGCACTCACCGACAGG | 4866 |
| 2122 | GGCACTGCACTCACCGACAG | 4865 |
| 2123 | AGGCACTGCACTCACCGACA | 4864 |
| 2124 | CAGGCACTGCACTCACCGAC | 4863 |
| 2125 | TCAGGCACTGCACTCACCGA | 4862 |
| 2126 | GTCAGGCACTGCACTCACCG | 4861 |
| 2127 | GTCCGGCAGAGGGACCCATGCTGACG | 4940 |
| 2128 | TCCGGCAGAGGGACCCATGC | 4941 |
| 2129 | CCGGCAGAGGGACCCATGCT | 4942 |
| 2130 | CGGCAGAGGGACCCATGCTG | 4943 |
| 2131 | GGTCCGGCAGAGGGACCCAT | 4939 |
| 2132 | TGGTCCGGCAGAGGGACCCA | 4938 |
| 2133 | GTGGTCCGGCAGAGGGACCC | 4937 |
| 2134 | TGTGGTCCGGCAGAGGGACC | 4936 |
| 2135 | GTGTGGTCCGGCAGAGGGAC | 4935 |
| 2136 | CGTGAGGCACATGTCCGTGTG | 3511 |
| 2137 | GTGAGGCACATGTCCGTGTG | 3512 |
| 2138 | TGAGGCACATGTCCGTGTGA | 3513 |
| 2139 | GAGGCACATGTCCGTGTGAC | 3514 |
| 2140 | AGGCACATGTCCGTGTGACC | 3515 |
| 2141 | GGCACATGTCCGTGTGACCT | 3516 |
| 2142 | GCACATGTCCGTGTGACCTG | 3517 |
| 2143 | CACATGTCCGTGTGACCTGC | 3518 |
| 2144 | ACATGTCCGTGTGACCTGCC | 3519 |
| 2145 | CATGTCCGTGTGACCTGCCT | 3520 |
| 2146 | ATGTCCGTGTGACCTGCCTG | 3521 |
| 2147 | TGTCCGTGTGACCTGCCTGT | 3522 |
| 2148 | GTCCGTGTGACCTGCCTGTC | 3523 |
| 2149 | TCCGTGTGACCTGCCTGTCC | 3524 |
| 2150 | CCGTGTGACCTGCCTGTCCC | 3525 |
| 2151 | CGTGTGACCTGCCTGTCCCT | 3526 |
| 2152 | ACGTGAGGCACATGTCCGTG | 3510 |
| 2153 | TACGTGAGGCACATGTCCGT | 3509 |
| 2154 | ATACGTGAGGCACATGTCCG | 3508 |
| 2155 | CATACGTGAGGCACATGTCC | 3507 |
| 2156 | GCATACGTGAGGCACATGTC | 3506 |
| 2157 | AGCATACGTGAGGCACATGT | 3505 |
| 2158 | AAGCATACGTGAGGCACATG | 3504 |
| 2159 | GAAGCATACGTGAGGCACAT | 3503 |
| 2160 | TGAAGCATACGTGAGGCACA | 3502 |
| 2161 | TTGAAGCATACGTGAGGCAC | 3501 |
| 2162 | CTTGAAGCATACGTGAGGCA | 3500 |
| 2163 | CCTTGAAGCATACGTGAGGC | 3499 |
| 2164 | CCCTTGAAGCATACGTGAGG | 3498 |
| 2165 | CCCCTTGAAGCATACGTGAG | 3497 |
| 2166 | GCCCCTTGAAGCATACGTGA | 3496 |
| 2167 | GGCCCCTTGAAGCATACGTG | 3495 |
| 2168 | GGGCCCCTTGAAGCATACGT | 3494 |
| 2169 | AGGGCCCCTTGAAGCATACG | 3493 |
| 2170 | CAGATGCAGCAAGCGGGCGGGAGAG | 798 |
| 2171 | CCAGATGCAGCAAGCGGGCG | 797 |
| 2172 | TCCAGATGCAGCAAGCGGGC | 796 |
| 2173 | GTCCAGATGCAGCAAGCGGG | 795 |
| 2174 | TGTCCAGATGCAGCAAGCGG | 794 |
| 2175 | GTGTCCAGATGCAGCAAGCG | 793 |
| 2176 | CCACGCTGCTGTCCCGCCAGCCCTGCAG | 848 |
| 2177 | CACGCTGCTGTCCCGCCAGC | 849 |
| 2178 | ACGCTGCTGTCCCGCCAGCC | 850 |
| 2179 | CGCTGCTGTCCCGCCAGCCC | 851 |
| 2180 | GCTGCTGTCCCGCCAGCCCT | 852 |
| 2181 | CTGCTGTCCCGCCAGCCCTG | 853 |
| 2182 | TGCTGTCCCGCCAGCCCTGC | 854 |
| 2183 | GCTGTCCCGCCAGCCCTGCA | 855 |
| 2184 | CTGTCCCGCCAGCCCTGCAG | 856 |
| 2185 | TGTCCCGCCAGCCCTGCAGC | 857 |
| 2186 | GTCCCGCCAGCCCTGCAGCC | 858 |
| 2187 | TCCCGCCAGCCCTGCAGCCC | 859 |
| 2188 | CCCGCCAGCCCTGCAGCCCA | 860 |
| 2189 | CCGCCAGCCCTGCAGCCCAG | 861 |
| 2190 | CGCCAGCCCTGCAGCCCAGA | 862 |
| 2191 | TCCACGCTGCTGTCCCGCCA | 847 |
| 2192 | GTCCACGCTGCTGTCCCGCC | 846 |
| 2193 | AGTCCACGCTGCTGTCCCGC | 845 |
| 2194 | GAGTCCACGCTGCTGTCCCG | 844 |
| 2195 | TGAGTCCACGCTGCTGTCCC | 843 |
| 2196 | CTGAGTCCACGCTGCTGTCC | 842 |
| 2197 | ACTGAGTCCACGCTGCTGTC | 841 |
| 2198 | GACTGAGTCCACGCTGCTGT | 840 |
| 2199 | AGACTGAGTCCACGCTGCTG | 839 |
| 2200 | GAGACTGAGTCCACGCTGCT | 838 |
| 2201 | GGAGACTGAGTCCACGCTGC | 837 |
| 2202 | AGGAGACTGAGTCCACGCTG | 836 |
| 2203 | TAGGAGACTGAGTCCACGCT | 835 |
| 2204 | CTAGGAGACTGAGTCCACGC | 834 |
| 2205 | CCTAGGAGACTGAGTCCACG | 833 |
| 2206 | ACCCGCCCCCACCCTGTGTGCCCCCCG | 1276 |
| 2207 | CCCGCCCCCACCCTGTGTGC | 1277 |
| 2208 | CCGCCCCCACCCTGTGTGCC | 1278 |
| 2209 | CGCCCCCACCCTGTGTGCCC | 1279 |
| 2210 | CACCCGCCCCCACCCTGTGT | 1275 |
| 2211 | CCACCCGCCCCCACCCTGTG | 1274 |
| 2212 | CCCACCCGCCCCCACCCTGT | 1273 |
| 2213 | CCCCACCCGCCCCCACCCTG | 1272 |
| 2214 | CCCCCACCCGCCCCCACCCT | 1271 |
| 2215 | CCCCCCACCCGCCCCCACCC | 1270 |
| 2216 | GCCCCCCACCCGCCCCCACC | 1269 |
| 2217 | AGCCCCCCACCCGCCCCCAC | 1268 |
| 2218 | CAGCCCCCCACCCGCCCCCA | 1267 |
| 2219 | GCAGCCCCCCACCCGCCCCC | 1266 |
| 2220 | AGCAGCCCCCCACCCGCCCC | 1265 |
| 2221 | CAGCAGCCCCCCACCCGCCC | 1264 |
| 2222 | CCAGCAGCCCCCCACCCGCC | 1263 |
| 2223 | CCCAGCAGCCCCCCACCCGC | 1262 |
| 2224 | ACCCAGCAGCCCCCCACCCG | 1261 |
| 2225 | CGCTCAGAGCCCGAGGCCTTTG | 1352 |
| 2226 | GCTCAGAGCCCGAGGCCTTT | 1353 |
| 2227 | CTCAGAGCCCGAGGCCTTTG | 1354 |
| 2228 | TCAGAGCCCGAGGCCTTTGC | 1355 |
| 2229 | CAGAGCCCGAGGCCTTTGCC | 1356 |
| 2230 | AGAGCCCGAGGCCTTTGCCC | 1357 |
| 2231 | GAGCCCGAGGCCTTTGCCCC | 1358 |
| 2232 | AGCCCGAGGCCTTTGCCCCT | 1359 |
| 2233 | GCCCGAGGCCTTTGCCCCTC | 1360 |
| 2234 | CCCGAGGCCTTTGCCCCTCC | 1361 |
| 2235 | CCGAGGCCTTTGCCCCTCCC | 1362 |
| 2236 | CGAGGCCTTTGCCCCTCCCT | 1363 |
| 2237 | CCGCTCAGAGCCCGAGGCCT | 1351 |
| 2238 | GCCGCTCAGAGCCCGAGGCC | 1350 |
| 2239 | GGCCGCTCAGAGCCCGAGGC | 1349 |
| 2240 | AGGCCGCTCAGAGCCCGAGG | 1348 |
| 2241 | AAGGCCGCTCAGAGCCCGAG | 1347 |
| 2242 | CAAGGCCGCTCAGAGCCCGA | 1346 |
| 2243 | CCAAGGCCGCTCAGAGCCCG | 1345 |
| 2244 | GCCAAGGCCGCTCAGAGCCC | 1344 |
| 2245 | GGCCAAGGCCGCTCAGAGCC | 1343 |
| 2246 | GGGCCAAGGCCGCTCAGAGC | 1342 |
| 2247 | AGGGCCAAGGCCGCTCAGAG | 1341 |
| 2248 | AAGGGCCAAGGCCGCTCAGA | 1340 |
| 2249 | GAAGGGCCAAGGCCGCTCAG | 1339 |
| 2250 | AGAAGGGCCAAGGCCGCTCA | 1338 |
| 2251 | GAGAAGGGCCAAGGCCGCTC | 1337 |

Hot Zones (Relative upstream location to gene start site)
700-900
1100-1400
1550-1700
2100-2300
3450-4300
4700-5000

### Examples

Genetic Code (5′ Upstream Region)

(SEQ ID NO: 11957)

GAGACATAACCATTGTACCTGCCTCCTAGGCTGTGAGGATTCACTGAGAT
GATCTTATAGTGCTTGCAACAATGTCTGGCACATAGGAAAAGTGATCACT
AAATGTTAGCCACGTCTTACTCCTGCAAGGCTCACCTCCCTGGAACCCAT
CGGTCCCAACCCTGCTCCTGAATCAGGCACAGTCCAGCTTGCAGCGGGAG
CAAAGGTCAGTACTCAGTGCCCCTGTCCCTTCCCCAGGCCAGAGGGGAGG
AGGAGACTGAGTCACGAATGACACCTCAGCCGCAGTTTGACCTCCAGGAC
TTACAGTCCTAGCAGCCGGTGCCACTAGCATGTGAGAGGTCCAGAGGCGC
TTCTGTCTCACCCGCCCGCCTGGGTGCACCCATGCTGGGAGCGCCTGCAC
CATTTGAGCATGTCCGAGAGCATCCACCAGAGTGTGTGTGGATTCACAGA
AGTGTGCAAATCACTAAGAACCAAGGGACTGGCACAGCCCATGCGTGCAC
CCACGCTCGCGAGGGGACCTGCTGCCTTTCAACGTGGCGGGGACGTGACC
TGTTAATGAATGTATTTACTTCCCAAAGTCTGAGGGTACGTTTTGCATCA
ATCTGTAGATGGATTTGTTTTGGGGAGCAGGGAGAGAATGAGAGCCCCCT
GTGCTCAGTCTTAGAGGGTGCAAGTAGCTGATGGGAAGAGCAGACTGCCT
TCCAGCCAGGCCTGGTCCTGTGAGTCAGGGACGTCCATCTTAGTGGGCAT
GAAAGGCCTGTGTGATCTCGAGGGAGACATCGCCTCTCCAAGCCTCTCCT
TATCTGTGCAACAGGCAGACTTAATGATTGGTGAGGCAATGAGGCTGATA
GCTCAGCATTAGCTACAGCCACCCCTCCTGGCCAACCACACAGGGATCAA
ACCAGGGGTCAGTCCAGAGGTCAGAGTCAGGAGCAGACAACTCAGATCCA
GCCAGGGACAGGCAGGTCACACGGACATGTGCCTCACGTATGCTTCAAGG
GGCCCTCCCCCGGGCAGAACTGAAGGACAGCTCCTGTTGCCATAGGAGGG
AGCTGGGTGAGATACTAGGAGGAACTTCCGGCATGATGATGTGTGATGAA
CAAGGGCCTCTGGCCAACAGGTCTGAATCAGGGCTGCCCAGCCCAGCCTG
GTGGGAAGGGCATGGAGCATGGGGGCTCATGTACTAAACCTCACCTGGAC
ACAAGGTGAAACAGCCCAACCCCAGAGGACCATTTTTGGCCCCGGATGGT
CAAATCCCCTCTTCCTCCCATCTACCACTGGCTTCTCCCTGGAGCAGTCT
TCATCCCAGGGGAGCCATGATGGGAGAGAGGGGCAGCGCAGGCTGGCCAC
CAAGAGATCCCCTGCCGGGGTGCAGGTTGGACTGTTGGTGAGGGGCCACA
GGTATTCTCAGGTACCAAGCCCTTGGAAGGAGACAAGGTACCAGGCTTCC
TGGAGGTGTGCTACATCTAGCTCAGCACCCTGCCAGGTCTCTCTACCCAC
ATGTCCTGACCTCCCTGGGTCCGTTGCCATGCGGGAGAGAGAGGCCAGGC
TCCTCCAGACCCTCTGCAGAGATGGAAAGGCTTGGAGGGTCTGGGGCCAC
GGGACCCCGCCAGCCCATTCTAGCACACCCGGGCCCATAGACCTTGTTGC
CTGCCCCTGCCTGGATCTGGGTCCCCACTGTGCCTTTGCCTCTGGGGCTA
TGGAGCAGGCCGCAGCAGAAGAGGAAAGGGCATCCCCAATACCAAATCCT
CCAGTGACCACTTCTTCACCTTCTACCCCACCACCAAAGTCTGCAGGAGA
CTTGAGACAGGTTTGTTCTGGGCGTGTGACTGATGCCTCTATAGGGGTCT
CAGTGCTCTAAGCCGTCTGGTATTTGCCTGGGGTGTGTGAAGACCTGGAT
TAAGGTTCCCAGCCTTACTACTAATGGGCTGTGCACTTGGAGCCCTTAGA
GCCTTAGGTTTCTAACCTATAAAATGGACTTAACGTCTACTTCACAGGGT
TCTATTTGCATTTTAACAGAAAACAAAGTCTTAAGTCAAAGGAATGAATC
TCTCTCTCTCTCTCTCTCTCTCTTTTTTAGACCAAGTCTAGCTCTGTCAC
TGGAGTGCAATGGTGCGATCTCTGCTCACTGCAACCTCCACCTCCGGGGT
TCAAGCAATTCTCGTGCCTCAGCCTCCTGAGTAGCTGGGACTACAGGCGT
GCATCACCATGCTCGGCTAATTTTTTGTATTTTTAGTAGAGACTGGGTTT
CGCCATGTTGCCCAGGCTGGTCTCGAACTCCTGGCCTCAGATATCCGTCC
GTCCCAGCCTCCCAAAGTGCTGGGATTACAGGCATGAGCCACTGTGCCCA
GCCAGGAATGGATCGCTAATAGAGGAATTCCAAGTCTCACCCACCGATAA
AGAATTCTGAGGGCAGAGCCGGGCCACTTTCTCAGGCCTCTGATTTCATA
CTGTGGTGTTAGTTACTTCTGAGAGGACAGCTTGCTGCCAGAGCTCTATT
TTTTATGTTAGAGGCTCCTTCTGCCTGCAGACTCTGCTGTCTGGGAAGGG
CACAGCGTTAGGAGGGAGAGGGAGGTGTGAGTCCCTCCATGGACCCGCTG
CTTTGTACTTCTCTATCTCATTTCCTTTTCAGCACCACTCTGGGCAATCA
GTATTCCAGCCCCATTTTATCCTCAGAAAATTGAGGCTCTGAGATGTTAT
CTCTGTGACCTGGGTCCTATTACGTGCCAAAGGCATCATTTAAGCCTAAG
ATGTCCTGGCTCCAAGGTGTCAGCATCTGGAAGACAGGCGCCCTCATCCT
GCCATCCCTGCTGCGGCTTCACTGTGGGCCCAGGGGACATCTCAGCCCCG
AGAAGGGTCAGCGGCCCCTCCTGGACCACCGACTCCCCGCAGAACTCCTC
TGTGCCCTCTCCTCACCAGACCTTGTTCCTCCCAGTTGCTCCCACAGCCA
GGGGGCAGTGAGGGCTGCTCTTCCCCCAGCCCCACTGAGGAACCCAGGAA
GGTGAACGAGAGAATCAGTCCTGGTGGGGGCTGGGGAGGGCCCCAGACAT
GAGACCAGCTCCTCCCCCAGGGGATGTTATCAGTGGGTCCAGAGGGCAAA
ATAGGGAGCCTGGTGGAGGGAGGGGCAAAGGCCTCGGGCTCTGAGCGGCC
TTGGCCCTTCTCCACCAACCCCTGCCCTACACTAAGGGGGAGGCAGCGGG
GGGCACACAGGGTGGGGGCGGGTGGGGGGCTGCTGGGTGAGCAGCACTCG
CCTGCCTGGATTGAAACCCAGAGATGGAGGTGCTGGGAGGGGCTGTGAGA
GCTCAGCCCTGTAACCAGGCCTTGCCGGAGCCACTGATGCCTGGTCTTCT
GTGCCTTTACTCCAAACACCCCCCAGCCCAAGCCACCCACTTGTTCTCAA
GTCTGAAGAAGCCCCTCACCCCTCTACTCCAGGCTGTGTTCAGGGCTTGG
GGCTGGTGGAGGGAGGGGCCTGAAATTCCAGTGTGAAAGGCTGAGATGGG
CCCGAGGCCCCTGGCCTATGTCCAAGCCATTTCCCCTCTCACCAGCCTCT
CCCTGGGGAGCCAGTCAGCTAGGAAGGAATGAGGGCTCCCCAGGCCCACC
CCCAGTTCCTGAGCTCATCTGGGCTGCAGGGCTGGCGGGACAGCAGCGTG
GACTCAGTCTCCTAGGGATTTCCCAACTCTCCCGCCCGCTTGCTGCATCT
GGACACCCTGCCTCAGGCCCTCATCTCCACTGGTCAGCAGGTGACCTTTG
CCCAGCGCCCTGGGTCCTCAGTGCCTGCTGCCCTGGAGATGATATAAAAC
AGGTCAGAACCCTCCTGCCTGTCTGCTCAGTTCATCCCTAGAGGCAGCTG
CTCCAGGTAATGCCCTCTGGGGAGGGGAAAGAGGAGGGGAGGAGGATGAA
GAGGGGCAAGAGGAGCTCCCTGCCCAGCCCAGCCAGCAAGCCTGGAGAAG
CACTTGCTAGAGCTAAGGAAGCCTCGGAGCTGGACGGGTGCCCCCCACCC
CTCATCATAACCTGAAGAACATGGAGGCCCGGGAGGGGTGTCACTTGCCC
AAAGCTACACAGGGGGTGGGGCTGGAAGTGGCTCCAAGTGCAGGTTCCCC
CCTCATTCTTCAGGCTTAGGGCTGGAGGAAGCCTTAGACAGCCCAGTCCT
ACCCCAGACAGGGAAACTGAGGCCTGGAGAGGGCCAGAAATCACCCAAAG
ACACACAGCATGTTGGCTGGACTGGACGGAGATCAGTCCAGACCGCAGGT
GCCTTGATGTTCAGTCTGGTGGGTTTTCTGCTCCATCCCACCCACCTCCC
TTTGGGCCTCGATCCCTCGCCCCTCACCAGTCCCCCTTCTGAGAGCCCGT
ATTAGCAGGGAGCCGGCCCCTACTCCTTCTGGCAGACCCAGCTAAGGTTC
TACCTTAGGGGCCACGCCACCTCCCCAGGGAGGGGTCCAGAGGCATGGGG
ACCTGGGGTGCCCCTCACAGGACACTTCCTTGCAGGAACAGAGGTGCCAT
G

9) APO B. Apolipoprotein B (ApoB) are the primary apolipoproteins of chylomicrons and low-density lipoproteins (LDL) and is required for lipoprotein formation during the transport of cholesterol to tissues. ApoB on the LDL particle acts as a ligand for LDL receptors in various cells throughout the body. High levels of ApoB can lead to plaques that cause vascular disease (atherosclerosis), leading to heart disease. There is considerable evidence that levels of ApoB are a better indicator of heart disease risk than total cholesterol or LDL (Contois et al, 2011; J. Clin. Lipid. 5 (4): 264-272).

There are two forms of ApoB (ApoB48 and ApoB100), with tissue regulated editing of ApoB48 and ApoB100 (reviewed in Davidson 2000; Ann. Rev. Nutr.; 20: 169-193). Editing is restricted to those transcripts expressed in the small intestine. This shorter version of the protein has a function specific to the small intestine. Editing results in a codon change creating an in frame stop codon leading to translation of a truncated protein, ApoB48. This stop codon results in the translation of a protein which lacks the carboxyl terminus which contains the protein's LDLR binding domain. The full protein ApoB100 which has nearly 4500 amino acid is present in VLDL and LDL. The main function of the full length liver expressed ApoB100 is as ligand for activation of the LDL-R. However editing results in a protein lacking this LDL-R binding region of the protein. This alters the function of the protein and the shorter ApoB48 protein as specific functions relative to the small intestine. ApoB48 is identical to the amino terminal 48% of ApoB100 (Knott et al., 1986; Nature 323 (6090): 734-8). The function of this isoform is in fat absorption of the small intestine and is involved in the synthesis, assembly and secretion of chylomicrons. These chylomicrons transport dietary lipids to tissues while the remaining chylomicrons along with associated residual lipids are in 2-3 hours taken up by the liver via the interaction of apolipoprotein E (ApoE) with lipoprotein receptors. It is the dominant ApoB protein in the small intestine of most mammals and the key protein in the exogenous pathway of lipoprotein metabolism.

Protein: ApoB Gene: APOB (Homo sapiens, chromosome 2, 21224301-21266945 [NCBI Reference Sequence: NC_{—}000002.11]; start site location: 21266817; strand: negative)

Gene Identification

| | |
|---|---|
| GeneID | 338 |
| HGNC | 603 |
| HPRD | 00133 |
| MIM | 107730 |

Targeted Sequences

| | | Relative |
|---|---|---|
| | | upstream |
| | | location |
| Sequence | | to gene |
| ID No: | Sequence (5′-3′) | start site |
| 2252 | CGGTGGGGCGGCTCCTGGGCTGC | 10 |
| 2329 | CCTCGCGGCCCTGGCTGGCTGGGCG | 46 |
| 2406 | AACCGAGAAGGGCACTCAGCCCCG | 88 |
| 2440 | CGGCGCCCGCACCCCATTTATAGG | 136 |
| 2451 | GTCCAAAGGGCGCCTCCCGGGCC | 195 |
| 2475 | CGTCTTCAGTGCTCTGGCGCGGCC | 341 |
| 2513 | CACCGGAAGCTTCAGCCAGCGCTCGCTG | 988 |
| 2552 | CGAGTGGGAGGCGGCCAGGAGCAAGCCG | 1281 |
| 2553 | CGTACACTCACGGAAATGCTGTAAAG | 2533 |
| 2576 | CGTCACAGCCAATAATGAGCGTACGC | 4862 |

Targeted Shift Sequences

| | | Relative |
|---|---|---|
| | | upstream |
| | | location |
| Sequence | | to gene |
| ID No: | Sequence (5′-3′) | start site |
| 2252 | CGGTGGGGCGGCTCCTGGGCTGC | 10 |
| 2253 | GGTGGGGCGGCTCCTGGGCT | 11 |
| 2254 | GTGGGGCGGCTCCTGGGCTG | 12 |
| 2255 | TGGGGCGGCTCCTGGGCTGC | 13 |
| 2256 | GGGGCGGCTCCTGGGCTGCG | 14 |
| 2257 | GGGCGGCTCCTGGGCTGCGG | 15 |
| 2258 | GGCGGCTCCTGGGCTGCGGC | 16 |
| 2259 | GCGGCTCCTGGGCTGCGGCC | 17 |
| 2260 | CGGCTCCTGGGCTGCGGCCT | 18 |
| 2261 | GGCTCCTGGGCTGCGGCCTG | 19 |
| 2262 | GCTCCTGGGCTGCGGCCTGG | 20 |
| 2263 | CTCCTGGGCTGCGGCCTGGC | 21 |
| 2264 | TCCTGGGCTGCGGCCTGGCC | 22 |
| 2265 | CCTGGGCTGCGGCCTGGCCT | 23 |
| 2266 | CTGGGCTGCGGCCTGGCCTC | 24 |
| 2267 | TGGGCTGCGGCCTGGCCTCG | 25 |
| 2268 | GGGCTGCGGCCTGGCCTCGG | 26 |
| 2269 | GGCTGCGGCCTGGCCTCGGC | 27 |
| 2270 | GCTGCGGCCTGGCCTCGGCC | 28 |
| 2271 | CTGCGGCCTGGCCTCGGCCT | 29 |
| 2272 | TGCGGCCTGGCCTCGGCCTC | 30 |
| 2273 | GCGGCCTGGCCTCGGCCTCG | 31 |
| 2274 | CGGCCTGGCCTCGGCCTCGC | 32 |
| 2275 | GGCCTGGCCTCGGCCTCGCG | 33 |
| 2276 | GCCTGGCCTCGGCCTCGCGG | 34 |
| 2277 | CCTGGCCTCGGCCTCGCGGC | 35 |
| 2278 | CTGGCCTCGGCCTCGCGGCC | 36 |
| 2279 | TGGCCTCGGCCTCGCGGCCC | 37 |
| 2280 | GGCCTCGGCCTCGCGGCCCT | 38 |
| 2281 | GCCTCGGCCTCGCGGCCCTG | 39 |
| 2282 | CCTCGGCCTCGCGGCCCTGG | 40 |
| 2283 | CTCGGCCTCGCGGCCCTGGC | 41 |
| 2284 | TCGGCCTCGCGGCCCTGGCT | 42 |
| 2285 | CGGCCTCGCGGCCCTGGCTG | 43 |
| 2286 | GGCCTCGCGGCCCTGGCTGG | 44 |
| 2287 | GCCTCGCGGCCCTGGCTGGC | 45 |
| 2288 | CCTCGCGGCCCTGGCTGGCT | 46 |
| 2289 | CTCGCGGCCCTGGCTGGCTG | 47 |
| 2290 | TCGCGGCCCTGGCTGGCTGG | 48 |
| 2291 | CGCGGCCCTGGCTGGCTGGG | 49 |
| 2292 | GCGGCCCTGGCTGGCTGGGC | 50 |
| 2293 | CGGCCCTGGCTGGCTGGGCG | 51 |
| 2294 | GGCCCTGGCTGGCTGGGCGG | 52 |
| 2295 | GCCCTGGCTGGCTGGGCGGG | 53 |
| 2296 | CCCTGGCTGGCTGGGCGGGC | 54 |
| 2297 | CCTGGCTGGCTGGGCGGGCT | 55 |
| 2298 | CTGGCTGGCTGGGCGGGCTC | 56 |
| 2299 | TGGCTGGCTGGGCGGGCTCC | 57 |
| 2300 | GGCTGGCTGGGCGGGCTCCT | 58 |
| 2301 | GCTGGCTGGGCGGGCTCCTC | 59 |
| 2302 | CTGGCTGGGCGGGCTCCTCA | 60 |
| 2303 | TGGCTGGGCGGGCTCCTCAG | 61 |
| 2304 | GGCTGGGCGGGCTCCTCAGC | 62 |
| 2305 | GCTGGGCGGGCTCCTCAGCG | 63 |
| 2306 | CTGGGCGGGCTCCTCAGCGG | 64 |
| 2307 | TGGGCGGGCTCCTCAGCGGC | 65 |
| 2308 | GGGCGGGCTCCTCAGCGGCA | 66 |
| 2309 | GGCGGGCTCCTCAGCGGCAG | 67 |
| 2310 | GCGGGCTCCTCAGCGGCAGC | 68 |
| 2311 | CGGGCTCCTCAGCGGCAGCA | 69 |
| 2312 | GGGCTCCTCAGCGGCAGCAA | 70 |
| 2313 | GGCTCCTCAGCGGCAGCAAC | 71 |
| 2314 | GCTCCTCAGCGGCAGCAACC | 72 |
| 2315 | CTCCTCAGCGGCAGCAACCG | 73 |
| 2316 | TCCTCAGCGGCAGCAACCGA | 74 |
| 2317 | CCTCAGCGGCAGCAACCGAG | 75 |
| 2318 | CTCAGCGGCAGCAACCGAGA | 76 |
| 2319 | TCAGCGGCAGCAACCGAGAA | 77 |
| 2320 | GCGGTGGGGCGGCTCCTGGG | 9 |
| 2321 | TGCGGTGGGGCGGCTCCTGG | 8 |
| 2322 | CTGCGGTGGGGCGGCTCCTG | 7 |
| 2323 | GCTGCGGTGGGGCGGCTCCT | 6 |
| 2324 | AGCTGCGGTGGGGCGGCTCC | 5 |
| 2325 | CAGCTGCGGTGGGGCGGCTC | 4 |
| 2326 | CCAGCTGCGGTGGGGCGGCT | 3 |
| 2327 | GCCAGCTGCGGTGGGGCGGC | 2 |
| 2328 | CGCCAGCTGCGGTGGGGCGG | 1 |
| 2329 | CCTCGCGGCCCTGGCTGGCTGGGCG | 46 |
| 2330 | CTCGCGGCCCTGGCTGGCTG | 47 |
| 2331 | TCGCGGCCCTGGCTGGCTGG | 48 |
| 2332 | CGCGGCCCTGGCTGGCTGGG | 49 |
| 2333 | GCGGCCCTGGCTGGCTGGGC | 50 |
| 2334 | CGGCCCTGGCTGGCTGGGCG | 51 |
| 2335 | GGCCCTGGCTGGCTGGGCGG | 52 |
| 2336 | GCCCTGGCTGGCTGGGCGGG | 53 |
| 2337 | CCCTGGCTGGCTGGGCGGGC | 54 |
| 2338 | CCTGGCTGGCTGGGCGGGCT | 55 |
| 2339 | CTGGCTGGCTGGGCGGGCTC | 56 |
| 2340 | TGGCTGGCTGGGCGGGCTCC | 57 |
| 2341 | GGCTGGCTGGGCGGGCTCCT | 58 |
| 2342 | GCTGGCTGGGCGGGCTCCTC | 59 |
| 2343 | CTGGCTGGGCGGGCTCCTCA | 60 |
| 2344 | TGGCTGGGCGGGCTCCTCAG | 61 |
| 2345 | GGCTGGGCGGGCTCCTCAGC | 62 |
| 2346 | GCTGGGCGGGCTCCTCAGCG | 63 |
| 2347 | CTGGGCGGGCTCCTCAGCGG | 64 |
| 2348 | TGGGCGGGCTCCTCAGCGGC | 65 |
| 2349 | GGGCGGGCTCCTCAGCGGCA | 66 |
| 2350 | GGCGGGCTCCTCAGCGGCAG | 67 |
| 2351 | GCGGGCTCCTCAGCGGCAGC | 68 |
| 2352 | CGGGCTCCTCAGCGGCAGCA | 69 |
| 2353 | GGGCTCCTCAGCGGCAGCAA | 70 |
| 2354 | GGCTCCTCAGCGGCAGCAAC | 71 |
| 2355 | GCTCCTCAGCGGCAGCAACC | 72 |
| 2356 | CTCCTCAGCGGCAGCAACCG | 73 |
| 2357 | TCCTCAGCGGCAGCAACCGA | 74 |
| 2358 | CCTCAGCGGCAGCAACCGAG | 75 |
| 2359 | CTCAGCGGCAGCAACCGAGA | 76 |
| 2360 | TCAGCGGCAGCAACCGAGAA | 77 |
| 2361 | GCCTCGCGGCCCTGGCTGGC | 45 |
| 2362 | GGCCTCGCGGCCCTGGCTGG | 44 |
| 2363 | CGGCCTCGCGGCCCTGGCTG | 43 |
| 2364 | TCGGCCTCGCGGCCCTGGCT | 42 |
| 2365 | CTCGGCCTCGCGGCCCTGGC | 41 |
| 2366 | CCTCGGCCTCGCGGCCCTGG | 40 |
| 2367 | GCCTCGGCCTCGCGGCCCTG | 39 |
| 2368 | GGCCTCGGCCTCGCGGCCCT | 38 |
| 2369 | TGGCCTCGGCCTCGCGGCCC | 37 |
| 2370 | CTGGCCTCGGCCTCGCGGCC | 36 |
| 2371 | CCTGGCCTCGGCCTCGCGGC | 35 |
| 2372 | GCCTGGCCTCGGCCTCGCGG | 34 |
| 2373 | GGCCTGGCCTCGGCCTCGCG | 33 |
| 2374 | CGGCCTGGCCTCGGCCTCGC | 32 |
| 2375 | GCGGCCTGGCCTCGGCCTCG | 31 |
| 2376 | TGCGGCCTGGCCTCGGCCTC | 30 |
| 2377 | CTGCGGCCTGGCCTCGGCCT | 29 |
| 2378 | GCTGCGGCCTGGCCTCGGCC | 28 |
| 2379 | GGCTGCGGCCTGGCCTCGGC | 27 |
| 2380 | GGGCTGCGGCCTGGCCTCGG | 26 |
| 2381 | TGGGCTGCGGCCTGGCCTCG | 25 |
| 2382 | CTGGGCTGCGGCCTGGCCTC | 24 |
| 2383 | CCTGGGCTGCGGCCTGGCCT | 23 |
| 2384 | TCCTGGGCTGCGGCCTGGCC | 22 |
| 2385 | CTCCTGGGCTGCGGCCTGGC | 21 |
| 2386 | GCTCCTGGGCTGCGGCCTGG | 20 |
| 2387 | GGCTCCTGGGCTGCGGCCTG | 19 |
| 2388 | CGGCTCCTGGGCTGCGGCCT | 18 |
| 2389 | GCGGCTCCTGGGCTGCGGCC | 17 |
| 2390 | GGCGGCTCCTGGGCTGCGGC | 16 |
| 2391 | GGGCGGCTCCTGGGCTGCGG | 15 |
| 2392 | GGGGCGGCTCCTGGGCTGCG | 14 |
| 2393 | TGGGGCGGCTCCTGGGCTGC | 13 |
| 2394 | GTGGGGCGGCTCCTGGGCTG | 12 |
| 2395 | GGTGGGGCGGCTCCTGGGCT | 11 |
| 2396 | CGGTGGGGCGGCTCCTGGGC | 10 |
| 2397 | GCGGTGGGGCGGCTCCTGGG | 9 |
| 2398 | TGCGGTGGGGCGGCTCCTGG | 8 |
| 2399 | CTGCGGTGGGGCGGCTCCTG | 7 |
| 2400 | GCTGCGGTGGGGCGGCTCCT | 6 |
| 2401 | AGCTGCGGTGGGGCGGCTCC | 5 |
| 2402 | CAGCTGCGGTGGGGCGGCTC | 4 |
| 2403 | CCAGCTGCGGTGGGGCGGCT | 3 |
| 2404 | GCCAGCTGCGGTGGGGCGGC | 2 |
| 2405 | CGCCAGCTGCGGTGGGGCGG | 1 |
| 2406 | AACCGAGAAGGGCACTCAGCCCCG | 88 |
| 2407 | ACCGAGAAGGGCACTCAGCC | 89 |
| 2408 | CCGAGAAGGGCACTCAGCCC | 90 |
| 2409 | CGAGAAGGGCACTCAGCCCC | 91 |
| 2410 | GAGAAGGGCACTCAGCCCCG | 92 |
| 2411 | AGAAGGGCACTCAGCCCCGC | 93 |
| 2412 | GAAGGGCACTCAGCCCCGCA | 94 |
| 2413 | AAGGGCACTCAGCCCCGCAG | 95 |
| 2414 | AGGGCACTCAGCCCCGCAGG | 96 |
| 2415 | GGGCACTCAGCCCCGCAGGT | 97 |
| 2416 | GGCACTCAGCCCCGCAGGTC | 98 |
| 2417 | GCACTCAGCCCCGCAGGTCC | 99 |
| 2418 | CACTCAGCCCCGCAGGTCCC | 100 |
| 2419 | ACTCAGCCCCGCAGGTCCCG | 101 |
| 2420 | CTCAGCCCCGCAGGTCCCGG | 102 |
| 2421 | TCAGCCCCGCAGGTCCCGGT | 103 |
| 2422 | CAGCCCCGCAGGTCCCGGTG | 104 |
| 2423 | AGCCCCGCAGGTCCCGGTGG | 105 |
| 2424 | GCCCCGCAGGTCCCGGTGGG | 106 |
| 2425 | CCCCGCAGGTCCCGGTGGGA | 107 |
| 2426 | CCCGCAGGTCCCGGTGGGAA | 108 |
| 2427 | CCGCAGGTCCCGGTGGGAAT | 109 |
| 2428 | CGCAGGTCCCGGTGGGAATG | 110 |
| 2429 | GCAGGTCCCGGTGGGAATGC | 111 |
| 2430 | CAGGTCCCGGTGGGAATGCG | 112 |
| 2431 | AGGTCCCGGTGGGAATGCGC | 113 |
| 2432 | GGTCCCGGTGGGAATGCGCG | 114 |
| 2433 | GTCCCGGTGGGAATGCGCGG | 115 |
| 2434 | TCCCGGTGGGAATGCGCGGC | 116 |
| 2435 | CCCGGTGGGAATGCGCGGCC | 117 |
| 2436 | CAACCGAGAAGGGCACTCAG | 87 |
| 2437 | GCAACCGAGAAGGGCACTCA | 86 |
| 2438 | AGCAACCGAGAAGGGCACTC | 85 |
| 2439 | CAGCAACCGAGAAGGGCACT | 84 |
| 2440 | CGGCGCCCGCACCCCATTTATAGG | 136 |
| 2441 | GGCGCCCGCACCCCATTTAT | 137 |
| 2442 | GCGCCCGCACCCCATTTATA | 138 |
| 2443 | CGCCCGCACCCCATTTATAG | 139 |
| 2444 | GCCCGCACCCCATTTATAGG | 140 |
| 2445 | CCCGCACCCCATTTATAGGA | 141 |
| 2446 | CCGCACCCCATTTATAGGAA | 142 |
| 2447 | CGCACCCCATTTATAGGAAG | 143 |
| 2448 | CCGGCGCCCGCACCCCATTT | 135 |
| 2449 | GCCGGCGCCCGCACCCCATT | 134 |
| 2450 | GGCCGGCGCCCGCACCCCAT | 133 |
| 2451 | GTCCAAAGGGCGCCTCCCGGGCC | 195 |
| 2452 | TCCAAAGGGCGCCTCCCGGG | 196 |
| 2453 | CCAAAGGGCGCCTCCCGGGC | 197 |
| 2454 | CAAAGGGCGCCTCCCGGGCC | 198 |
| 2455 | AAAGGGCGCCTCCCGGGCCT | 199 |
| 2456 | AAGGGCGCCTCCCGGGCCTG | 200 |
| 2457 | AGGGCGCCTCCCGGGCCTGA | 201 |
| 2458 | GGGCGCCTCCCGGGCCTGAC | 202 |
| 2459 | GGCGCCTCCCGGGCCTGACC | 203 |
| 2460 | GCGCCTCCCGGGCCTGACCT | 204 |
| 2461 | CGCCTCCCGGGCCTGACCTG | 205 |
| 2462 | GCCTCCCGGGCCTGACCTGT | 206 |
| 2463 | CCTCCCGGGCCTGACCTGTT | 207 |
| 2464 | CTCCCGGGCCTGACCTGTTT | 208 |
| 2465 | TCCCGGGCCTGACCTGTTTG | 209 |
| 2466 | CCCGGGCCTGACCTGTTTGC | 210 |
| 2467 | CCGGGCCTGACCTGTTTGCT | 211 |
| 2468 | CGGGCCTGACCTGTTTGCTT | 212 |
| 2469 | GGTCCAAAGGGCGCCTCCCG | 194 |
| 2470 | AGGTCCAAAGGGCGCCTCCC | 193 |
| 2471 | AAGGTCCAAAGGGCGCCTCC | 192 |
| 2472 | AAAGGTCCAAAGGGCGCCTC | 191 |
| 2473 | AAAAGGTCCAAAGGGCGCCT | 190 |
| 2474 | CAAAAGGTCCAAAGGGCGCC | 189 |
| 2475 | CGTCTTCAGTGCTCTGGCGCGGCC | 341 |
| 2476 | GTCTTCAGTGCTCTGGCGCG | 342 |
| 2477 | TCTTCAGTGCTCTGGCGCGG | 343 |
| 2478 | CTTCAGTGCTCTGGCGCGGC | 344 |
| 2479 | TTCAGTGCTCTGGCGCGGCC | 345 |
| 2480 | TCAGTGCTCTGGCGCGGCCC | 346 |
| 2481 | CAGTGCTCTGGCGCGGCCCT | 347 |
| 2482 | AGTGCTCTGGCGCGGCCCTT | 348 |
| 2483 | GTGCTCTGGCGCGGCCCTTC | 349 |
| 2484 | TGCTCTGGCGCGGCCCTTCC | 350 |
| 2485 | GCTCTGGCGCGGCCCTTCCT | 351 |
| 2486 | CTCTGGCGCGGCCCTTCCTG | 352 |
| 2487 | TCTGGCGCGGCCCTTCCTGT | 353 |
| 2488 | CTGGCGCGGCCCTTCCTGTG | 354 |
| 2489 | TGGCGCGGCCCTTCCTGTGT | 355 |
| 2490 | GGCGCGGCCCTTCCTGTGTC | 356 |
| 2491 | GCGCGGCCCTTCCTGTGTCT | 357 |
| 2492 | CGCGGCCCTTCCTGTGTCTC | 358 |
| 2493 | GCGGCCCTTCCTGTGTCTCA | 359 |
| 2494 | CGGCCCTTCCTGTGTCTCAG | 360 |
| 2495 | GCGTCTTCAGTGCTCTGGCG | 340 |
| 2496 | AGCGTCTTCAGTGCTCTGGC | 339 |
| 2497 | AAGCGTCTTCAGTGCTCTGG | 338 |
| 2498 | CAAGCGTCTTCAGTGCTCTG | 337 |
| 2499 | CCAAGCGTCTTCAGTGCTCT | 336 |
| 2500 | CCCAAGCGTCTTCAGTGCTC | 335 |
| 2501 | CCCCAAGCGTCTTCAGTGCT | 334 |
| 2502 | TCCCCAAGCGTCTTCAGTGC | 333 |
| 2503 | TTCCCCAAGCGTCTTCAGTG | 332 |
| 2504 | CTTCCCCAAGCGTCTTCAGT | 331 |
| 2505 | CCTTCCCCAAGCGTCTTCAG | 330 |
| 2506 | CCCTTCCCCAAGCGTCTTCA | 329 |
| 2507 | TCCCTTCCCCAAGCGTCTTC | 328 |
| 2508 | TTCCCTTCCCCAAGCGTCTT | 327 |
| 2509 | GTTCCCTTCCCCAAGCGTCT | 326 |
| 2510 | GGTTCCCTTCCCCAAGCGTC | 325 |
| 2511 | GGGTTCCCTTCCCCAAGCGT | 324 |
| 2512 | TGGGTTCCCTTCCCCAAGCG | 323 |
| 2513 | CACCGGAAGCTTCAGCCAGCGCTCGCTG | 988 |
| 2514 | ACCGGAAGCTTCAGCCAGCG | 989 |
| 2515 | CCGGAAGCTTCAGCCAGCGC | 990 |
| 2516 | CGGAAGCTTCAGCCAGCGCT | 991 |
| 2517 | GGAAGCTTCAGCCAGCGCTC | 992 |
| 2518 | GAAGCTTCAGCCAGCGCTCG | 993 |
| 2519 | AAGCTTCAGCCAGCGCTCGC | 994 |
| 2520 | AGCTTCAGCCAGCGCTCGCT | 995 |
| 2521 | GCTTCAGCCAGCGCTCGCTG | 996 |
| 2522 | CTTCAGCCAGCGCTCGCTGC | 997 |
| 2523 | TTCAGCCAGCGCTCGCTGCC | 998 |
| 2524 | TCAGCCAGCGCTCGCTGCCT | 999 |
| 2525 | CAGCCAGCGCTCGCTGCCTC | 1000 |
| 2526 | AGCCAGCGCTCGCTGCCTCT | 1001 |
| 2527 | GCCAGCGCTCGCTGCCTCTG | 1002 |
| 2528 | CCAGCGCTCGCTGCCTCTGC | 1003 |
| 2529 | CAGCGCTCGCTGCCTCTGCC | 1004 |
| 2530 | AGCGCTCGCTGCCTCTGCCC | 1005 |
| 2531 | GCGCTCGCTGCCTCTGCCCA | 1006 |
| 2532 | CGCTCGCTGCCTCTGCCCAG | 1007 |
| 2533 | GCTCGCTGCCTCTGCCCAGC | 1008 |
| 2534 | CTCGCTGCCTCTGCCCAGCT | 1009 |
| 2535 | TCGCTGCCTCTGCCCAGCTG | 1010 |
| 2536 | CGCTGCCTCTGCCCAGCTGG | 1011 |
| 2537 | CCACCGGAAGCTTCAGCCAG | 987 |
| 2538 | CCCACCGGAAGCTTCAGCCA | 986 |
| 2539 | TCCCACCGGAAGCTTCAGCC | 985 |
| 2540 | TTCCCACCGGAAGCTTCAGC | 984 |
| 2541 | TTTCCCACCGGAAGCTTCAG | 983 |
| 2542 | ATTTCCCACCGGAAGCTTCA | 982 |
| 2543 | CATTTCCCACCGGAAGCTTC | 981 |
| 2544 | CCATTTCCCACCGGAAGCTT | 980 |
| 2545 | CCCATTTCCCACCGGAAGCT | 979 |
| 2546 | GCCCATTTCCCACCGGAAGC | 978 |
| 2547 | TGCCCATTTCCCACCGGAAG | 977 |
| 2548 | CTGCCCATTTCCCACCGGAA | 976 |
| 2549 | ACTGCCCATTTCCCACCGGA | 975 |
| 2550 | CACTGCCCATTTCCCACCGG | 974 |
| 2551 | GCACTGCCCATTTCCCACCG | 973 |
| 2552 | CGAGTGGGAGGCGGCCAGGAGCAAGCCG | 1281 |
| 2553 | CGTACACTCACGGAAATGCTGTAAAG | 2533 |
| 2554 | GTACACTCACGGAAATGCTG | 2534 |
| 2555 | TACACTCACGGAAATGCTGT | 2535 |
| 2556 | ACACTCACGGAAATGCTGTA | 2536 |
| 2557 | CACTCACGGAAATGCTGTAA | 2537 |
| 2558 | GCGTACACTCACGGAAATGC | 2532 |
| 2559 | TGCGTACACTCACGGAAATG | 2531 |
| 2560 | TTGCGTACACTCACGGAAAT | 2530 |
| 2561 | CTTGCGTACACTCACGGAAA | 2529 |
| 2562 | ACTTGCGTACACTCACGGAA | 2528 |
| 2563 | GACTTGCGTACACTCACGGA | 2527 |
| 2564 | TGACTTGCGTACACTCACGG | 2526 |
| 2565 | CTGACTTGCGTACACTCACG | 2525 |
| 2566 | GCTGACTTGCGTACACTCAC | 2524 |
| 2567 | AGCTGACTTGCGTACACTCA | 2523 |
| 2568 | GAGCTGACTTGCGTACACTC | 2522 |
| 2569 | TGAGCTGACTTGCGTACACT | 2521 |
| 2570 | TTGAGCTGACTTGCGTACAC | 2520 |
| 2571 | GTTGAGCTGACTTGCGTACA | 2519 |
| 2572 | TGTTGAGCTGACTTGCGTAC | 2518 |
| 2573 | TTGTTGAGCTGACTTGCGTA | 2517 |
| 2574 | ATTGTTGAGCTGACTTGCGT | 2516 |
| 2575 | AATTGTTGAGCTGACTTGCG | 2515 |
| 2576 | CGTCACAGCCAATAATGAGCGTACGC | 4862 |
| 2577 | GTCACAGCCAATAATGAGCG | 4863 |
| 2578 | TCACAGCCAATAATGAGCGT | 4864 |
| 2579 | CACAGCCAATAATGAGCGTA | 4865 |
| 2580 | ACAGCCAATAATGAGCGTAC | 4866 |
| 2581 | CAGCCAATAATGAGCGTACG | 4867 |
| 2582 | AGCCAATAATGAGCGTACGC | 4868 |
| 2583 | GCCAATAATGAGCGTACGCA | 4869 |
| 2584 | CCAATAATGAGCGTACGCAA | 4870 |
| 2585 | ACGTCACAGCCAATAATGAG | 4861 |
| 2586 | GACGTCACAGCCAATAATGA | 4860 |
| 2587 | AGACGTCACAGCCAATAATG | 4859 |
| 2588 | CAGACGTCACAGCCAATAAT | 4858 |
| 2589 | TCAGACGTCACAGCCAATAA | 4857 |
| 2590 | ATCAGACGTCACAGCCAATA | 4856 |
| 2591 | AATCAGACGTCACAGCCAAT | 4855 |
| 2592 | TAATCAGACGTCACAGCCAA | 4854 |
| 2593 | ATAATCAGACGTCACAGCCA | 4853 |
| 2594 | CATAATCAGACGTCACAGCC | 4852 |
| 2595 | GCATAATCAGACGTCACAGC | 4851 |
| 2596 | GGCATAATCAGACGTCACAG | 4850 |
| 2597 | GGGCATAATCAGACGTCACA | 4849 |
| 2598 | AGGGCATAATCAGACGTCAC | 4848 |
| 2599 | AAGGGCATAATCAGACGTCA | 4847 |
| 2600 | GAAGGGCATAATCAGACGTC | 4846 |

```
Hot Zones (Relative upstream location to gene start site)
  1-600
  700-1400
2450-2650
3450-3700
4600-5000
```

Genetic Code (5′ Upstream Region)

(SEQ ID NO: 11958)

TGCATATGAAAGAAACCTATTCACATGGACCATATTACATTATAATCACA
GTGTTTACTGCTTGACTACCATCTGCCTGGCCTAGCAAGGGTGTCAGTGA
GGAAGAGAGGACAAGGGGTACCAATCTGTGAACTACACATGGTTCTTGCT
CTCCCAGCTTCTCTCTCCCATTGGCAAGGCAACAGGTAAACACATGAAAA
ATCAAATAATGCTATAAGAGAAAAATGTATTCAGGACAACAACAGGTTTG
TATGAAGGCCTTTCATCATCGTTGTCCTACCTAGAAACTGAATGACAGGG
AATCAGAGTCACAAGCTATGAAGTCTAACTGGGCTGTTCCCAGAGAAAGA
TTCAGTGCAGTAGGTGGGGCTGCAGCCAGCCCTGGGTGGGTGGAAGGATG
ACATCCACATAGGCAAGAGGGTGATAATTCACTTACGCAGCTCCTCACTG
CACATTGAACCCTGCTGACTTCTGGCTTCTCTCCCGGGAGGAACTGCGAC
TCAACATTCTGACCTTATCTCTTGGGTAGCAGAATGATGGAGAAGGAAAG
TTTCTTTTTGCTTCTCGCAGGGGTTAATCATCCATCTGGAATGCCTACAT
TTGGTTGACAATGGCTCACCCTATCATCTTCCTCCTGAACCATTCACCTA
AATGTGCCATTTCTTTCCTGATAGTTCTCATTTGTGTGTGTGTGTGTGTG
TGTGTGTGTGTGCACGTGCTCACACATGCATGCTGTCACTGGGTAAACAG
GCCACCCTGGGCACAGTTCCATCTACAATGTTTGAAGTTTACTTTCCAGC
TTCTGGGCATCATTTGCAATTATAATGCTGTCATAGGCAGAAACGAGATA
GGCTAATTAATCGTTGTCAATACTGATCCCTATTTGCCAGATGAGATTTT
GGAGCAGCATGGCTGGGAATAATTGGTATAGACTGTATTTCCTTGCTTTA
TGTCACTGGAAATATTTATTTAAGCATCACGGTCGCTATGCATAAATATC
CTGGAAAATGGGGTATAGCTGAATGGTGCAGATTCATTCATTCATATTCA
GCAAATTATGTTCTAAGCACCTACTTCAGTATGTGAACAGCACTAAACTC
AGAATATTGGTCTGCTGGGGTCCTTTATTAGCTTCCATGATTCCCTGAAC
TTGGCCAAGACCCTTCTGGTCGGCTGCAGATAGGCACAATGGATAGTTTT
GCTTCTAGATAATGTAACTGGGACATTCAGCATTATCTATCGCCTTGAAA
TTCCTCTAGTCAGGTGGCTTTCTAATGGGTACCCAGAGCCCTATGACTAC
CCAGATTGATGGTGCACCCAACAGGACTTTGCATTTATGAGCTGATAAGT
CACAGTCACTAGCTGAGATTAATCTGTGTGACACCAGAATGTGTCTCTAT
CTAAAGGAAAAGGGATGAAGGGTGATATCTTTGGTCACAAGTAATGTATT
TCCATGTAGTCTTTGACAAAGGATCTAAGTGGATTTTGTAATTGAAGAAA
AATCTATGCACTAATCTTTACAGCATTTCCGTGAGTGTACGCAAGTCAGC
TCAACAATTCAACATTTGCTCTGTGGGGTTGTGCTAGACCCTGTCAGGGG
ATAACTACTGCTGGCTGGGGCCCAGTTCAGGGAAGACTTGCCAAAGACCA
TCAGGAAAAGAGGGAAGCTGAGTCTTAGGTTTCTTCCTTTAGAGATGGTG
ACAGTCCTCTCACCACCTCCAAGCATCTCACAATGTTTCCCTGCCTCCAA
GTCATCAAATTCATTTTTGATTCCTACTTCATAAAAATTACATTCTCCCA
GCACTTTGGGAGGCCAAGGCGGGCAGATCATGAGGTCAGGAGTTCAAGAC
CAGCCTGATCAACATGGTGAAACACCGTCTCTACTAAAAATACAAAAATT
AGCTGGGCATAGTGGCACTCACCTGTTATCTCAGTTACTTGGGAGGCTAA
GGCAGGAGAATCGCTTAAACCCGGGAGGCAGAGGTTGCAGTGAGCCGAGA
TTGTACCACTACACTCCAGCCTGGGTGACAGAGGGAGACTCCATCTCAAA
TAAATAAATTAAAAAAAAAAATATATATATATATGTATATATTCTCTATG
GATGCTGACCATTGGACCCTGGTTTCATCTGCACGTAACAGAGTAAGCTT
GGACTTGTGCTTGTAAATTAAAGCTCGACACCTCCTTTTGGCTTCTCTAT
ACCTGAATATTCTTACTCACTCTCCTTAATGTGAATATGCATGGAAGCAG
GACCATTTCCTCAAACACTAGCAGCAGCGAACCCTGTGGAAAGTCAGTCC
ACATAGAATAATTCAAATAAAGTGTTCAGAGAAATGGGGTTTCAGAGCAA
TTACTTTTTCCAGACCTTTCACAAATCAGTGGTGTAGGTATGACCAGCCT
TGAGTTGAGACCTCTGTAATATCCATCTTTAATAACATTAATATGCTGTG
GATGAGCAACTGATCACTGGAGGGAGTTTAGCTGCCCATAGGAGTTCATG
GCTAATGACAATATCTGAATAAGGACAGGTGTGGAGCCCAGGTGCAGGAA
GCAGGCGAAGGTCTTTCTGTGAGTCTCCTCTGAGGGAACTGGGTCTTTAT
ACATAGTTACTGTTTCAGAATTGATCCTTCTGGAATCATCAGTCTTCACC
AGTAGCTTGTTACATCTGGGGTTATCTCATAATTCAAACAAAGCTGACAA
GTTGTAACAATGAGCACACACTGACTTCTGCAACAGGCGCTGTCCACTTC
CCATCCGCACTCTACCGGCTTGCTCCTGGCCGCCTCCCACTCGCCTTCCT
GGGTGGTCCCCCAGCAGTTATACCTACCTGGTTGTCGCCCCCTCTATCCT
ACCACAATTGCTCACTAGCGGTTTCCTGCGTACACAGCTTGTCTCCCTAA
CCAGAGTGGAGGTGCCTTGGGGACACAGCCAGGCTCAGACATTCACTCAG
CTCATCATAGTGCCATCCCATCAATAACCCCTTCTGAGTGATCCTGGGTT
AGTAAACCGAGTGTCCCTGAAATTCCACTACCGCTGATTCCCTCCAGCTG
GGCAGAGGCAGCGAGCGCTGGCTGAAGCTTCCGGTGGGAAATGGGCAGTG
CCTAGAAGAGAAGGAAACGATGCATGAGAAGGTTCCAGATGTCTATGAGG
AACATGACGTGTCCTGTCCACTACTCTGCTTTTCCTCGTCCGCCTCCCCA
CCACTGGAGGAAACCTAGAAGCTGGTGCAGGAAATCCTCCTCTCAACAAC
CCAAGAACACTTTGCACAAGAGGGGTGCGCCCTCGGAGGTTGCTCTTCCC
CAGAGGCCTCTCCTCGCTGGGGTTTCTTGAAGACAGATACTTGGACTCCT
GCTGGGACCAGGCAGGCCACCCATCCTCAGGGGCAGTGACTGGTCACTCA
CCAGACCTCCCTGCATCCCCCTTCTCTCTCCTCCCCCAGCACGGGCTGAA
CCCCGCAGCCACAGATTCTGATCAGGATTAGGGTGTGGGTGCAAATCCAA
GGTCCACCAAAATGGAAAAGAAGTAACCGATGGGAACACGTCTCCACCAA
GACAGCGCTCAGGACTGGTTCTCCTCGTGGCTCCCAATTCAGTCCAGGAG
AAGCAGAGATTTTGTCCCCATGGTGGGTCATCTGAAGAAGGCACCCCTGG
TCAGGGCAGGCTTCTCAGACCCTGAGGCGCTGGCCATGGCCCCACTGAGA
CACAGGAAGGGCCGCGCCAGAGCACTGAAGACGCTTGGGGAAGGGAACCC
ACCTGGGACCCAGCCCCTGGTGGCTGCGGCTGCATCCCAGGTGGGCCCCC
TCCCCGAGGCTCTTCAAGGCTCAAAGAGAAGCCAGTGTAGAAAAGCAAAC
AGGTCAGGCCCGGGAGGCGCCCTTTGGACCTTTTGCAATCCTGGCGCTCT
TGCAGCCTGGGCTTCCTATAAATGGGGTGCGGGCGCCGGCCGCGCATTCC
CACCGGGACCTGCGGGGCTGAGTGCCCTTCTCGGTTGCTGCCGCTGAGGA
GCCCGCCCAGCCAGCCAGGGCCGCGAGGCCGAGGCCAGGCCGCAGCCCAG
GAGCCGCCCCACCGCAGCTGGCGATG

10) IL17. Interleukin 17 is a cytokine is a potent mediator in delayed-type reactions by increasing chemokine production in various tissues to recruit monocytes and neutrophils to the site of inflammation. IL-17 is produced by T-helper cells and is induced by IL-23 which results in destructive tissue damage in delayed-type reactions. Interleukin 17 as a family functions as a proinflammatory cytokine that responds to the invasion of the immune system by extracellular pathogens and induces destruction of the pathogen's cellular matrix. Interleukin 17 acts synergistically with tumor necrosis factor and interleukin-1 (Chiricozzi et al., J Invest Dermatol. 2011 March; 131(3):677-87, Miossec et al., N. Engl. J. Med. 361 (9): 888-98). Most notably IL is involved in inducing many immune signaling molecules and mediating proinflammatory responses (e.g. allergic responses). IL-17 induces the production of many other cytokines (such as IL-6, G-CSF, GM-CSF, IL-1β, TGF-β, TNF-α), chemokines (including IL-8, GRO-α, and MCP-1), and prostaglandins (e.g., PGE2) from many cell types (fibroblasts, endothelial cells, epithelial cells, keratinocytes, and macrophages). The release of cytokines causes many functions, such as airway remodeling, a characteristic of IL-17 responses. The increased expression of chemokines attracts other cells including neutrophils. IL-17 function is also essential to a subset of CD4+ T-Cells called T helper 17 (Th17) cells. As a result of these roles, the IL-17 family has been linked to many immune/autoimmune related diseases including rheumatoid arthritis, psoriasis, ankylosing spondylitis asthma, lupus, allograft rejection and anti-tumor immunity (reviewed in Miossec and Kolls, Nature Reviews Drug Discovery 11, 763-776).

Protein: IL17 Gene: IL17A (Homo sapiens, chromosome 6, 52051185-52055436 [NCBI Reference Sequence: NC_{—}000006.11]; start site location: 52051230; strand: positive)

Targeted Sequences

| | | Relative |
|---|---|---|
| | | upstream |
| | | location |
| Sequence | | to gene |
| ID No: | Sequence (5′-3′) | start site |
| 2601 | CTTGTTTGTATCCGCATGGCTGTGCTC | 4451 |
| 2616 | CGAGACCGTTGAGGTGGAGTG | 3148 |
| 2635 | GGTCACTTACGTGGCGTGTCGC | 107 |
| 2664 | GACAAAATGTAGCGCTATCG | 55 |

Target Shift Sequences

| | | Relative |
|---|---|---|
| | | upstream |
| | | location |
| Sequence ID | | to gene |
| No: | Sequence (5′-3′) | start site |
| 2601 | CTTGTTTGTATCCGCATGGCTGTGCTC | 4451 |
| 2602 | TTGTTTGTATCCGCATGGCT | 4452 |
| 2603 | TGTTTGTATCCGCATGGCTG | 4453 |
| 2604 | GTTTGTATCCGCATGGCTGT | 4454 |
| 2605 | TTTGTATCCGCATGGCTGTG | 4455 |
| 2606 | TTGTATCCGCATGGCTGTGC | 4456 |

Gene Identification

| | |
|---|---|
| GeneID | 3605 |
| HGNC | 5981 |
| HPRD | 04396 |
| MIM | 603149 |

| | | |
|---|---|---|
| 2607 | TGTATCCGCATGGCTGTGCT | 4457 |
| 2608 | GTATCCGCATGGCTGTGCTC | 4458 |
| 2609 | TATCCGCATGGCTGTGCTCC | 4459 |
| 2610 | ATCCGCATGGCTGTGCTCCT | 4460 |
| 2611 | TCCGCATGGCTGTGCTCCTG | 4461 |
| 2612 | CCGCATGGCTGTGCTCCTGA | 4462 |
| 2613 | CGCATGGCTGTGCTCCTGAG | 4463 |
| 2614 | GCTTGTTTGTATCCGCATGG | 4450 |
| 2615 | TGCTTGTTTGTATCCGCATG | 4449 |
| 2616 | CGAGACCGTTGAGGTGGAGTG | 3148 |
| 2617 | CCGAGACCGTTGAGGTGGAG | 3147 |
| 2618 | TCCGAGACCGTTGAGGTGGA | 3146 |
| 2619 | ATCCGAGACCGTTGAGGTGG | 3145 |
| 2620 | AATCCGAGACCGTTGAGGTG | 3144 |
| 2621 | CAATCCGAGACCGTTGAGGT | 3143 |
| 2622 | TCAATCCGAGACCGTTGAGG | 3142 |
| 2623 | TTCAATCCGAGACCGTTGAG | 3141 |
| 2624 | TTTCAATCCGAGACCGTTGA | 3140 |
| 2625 | GTTTCAATCCGAGACCGTTG | 3139 |
| 2626 | GGTTTCAATCCGAGACCGTT | 3138 |
| 2627 | AGGTTTCAATCCGAGACCGT | 3137 |
| 2628 | CAGGTTTCAATCCGAGACCG | 3136 |
| 2629 | TCAGGTTTCAATCCGAGACC | 3135 |
| 2630 | CTCAGGTTTCAATCCGAGAC | 3134 |
| 2631 | ACTCAGGTTTCAATCCGAGA | 3133 |
| 2632 | GACTCAGGTTTCAATCCGAG | 3132 |
| 2633 | TGACTCAGGTTTCAATCCGA | 3131 |
| 2634 | CTGACTCAGGTTTCAATCCG | 3130 |
| 2635 | GGTCACTTACGTGGCGTGTCGC | 107 |
| 2636 | GTCACTTACGTGGCGTGTCG | 108 |
| 2637 | TCACTTACGTGGCGTGTCGC | 109 |
| 2638 | CACTTACGTGGCGTGTCGCA | 110 |
| 2639 | ACTTACGTGGCGTGTCGCAG | 111 |
| 2640 | CTTACGTGGCGTGTCGCAGT | 112 |
| 2641 | TTACGTGGCGTGTCGCAGTG | 113 |
| 2642 | TACGTGGCGTGTCGCAGTGG | 114 |
| 2643 | ACGTGGCGTGTCGCAGTGGG | 115 |
| 2644 | CGTGGCGTGTCGCAGTGGGT | 116 |
| 2645 | GTGGCGTGTCGCAGTGGGTT | 117 |
| 2646 | TGGCGTGTCGCAGTGGGTTC | 118 |
| 2647 | GGCGTGTCGCAGTGGGTTCA | 119 |
| 2648 | GCGTGTCGCAGTGGGTTCAG | 120 |
| 2649 | CGTGTCGCAGTGGGTTCAGG | 121 |
| 2650 | GTGTCGCAGTGGGTTCAGGG | 122 |
| 2651 | TGTCGCAGTGGGTTCAGGGG | 123 |
| 2652 | GTCGCAGTGGGTTCAGGGGT | 124 |
| 2653 | TCGCAGTGGGTTCAGGGGTG | 125 |
| 2654 | CGCAGTGGGTTCAGGGGTGA | 126 |
| 2655 | TGGTCACTTACGTGGCGTGT | 106 |
| 2656 | GTGGTCACTTACGTGGCGTG | 105 |
| 2657 | TGTGGTCACTTACGTGGCGT | 104 |
| 2658 | CTGTGGTCACTTACGTGGCG | 103 |
| 2659 | TCTGTGGTCACTTACGTGGC | 102 |
| 2660 | TTCTGTGGTCACTTACGTGG | 101 |
| 2661 | CTTCTGTGGTCACTTACGTG | 100 |
| 2662 | CCTTCTGTGGTCACTTACGT | 99 |
| 2663 | TCCTTCTGTGGTCACTTACG | 98 |
| 2664 | GACAAAATGTAGCGCTATCG | 55 |
| 2665 | GGACAAAATGTAGCGCTATC | 54 |

Hot Zones (Relative upstream location to gene start site)
1-150
2900-3250
4250-4600

### Examples

Genetic Code (5′ Upstream Region)

(SEQ ID NO: 11959)

CACTGTTCAGGACGGCCCTCAGGAGCACAGCCATGCGGATACAAACAAGC
ATTATGCTGGAGAGGAACAATGATGCTATCAGGTTGGTACCAAGACTGAT
ACTTCCTATTTAAGAAATCATGTTAATTATATAGGTATTAAGTTCTGGTT
TTTGCAAGACAACATCGGTAACCTCAGAGATGAACTTCTAGTATGTAATA
GTATTGATATCTTCAAATTCAGGGTAGATGATCTAGAGCTTGATAAACTG
GGTCTAAATCTGAGTCTCAATTAAATCCTGATTTTATCAACTCCCCAAGG
CCTTCCAGATGGCTCTCTATTGCAAATCTTCTCCTCTCTAAACATCACGT
CTTCCTTTTAAATGTCTGTTTGACATTGCTCCTTGTGTGCTGGCCCATTT
GCAAAGTATCAGCCCCTACATTTAAAATCCAAGAGAATTCTCTCCAACGA
CTCATTTATTCTCTCTATATTAAGTGAAGATCAAGAGAGTAGTCTTGCCT
GGTGATGTGCACAGCATTAATCAAAATGGCTCCAATCTTTTTGAACAGCC
ACCATCTTTTATTAACTTTAAGCAGAAACAATGGCATGATGCCTCCTCCT
ATACTGAGACCTTTGTTACTTTTATAAAATGGCTATAGAATATAAATTTA
AAATATATTAATCTCTCAATGGTAACCTTACTACCTAAGCAAAAATTAAA
ATTTATTTATTTTATCTTTTAGCAAGCCTTAATTTTCCACCACACTGAGA
CAAAAGACTCAAAAATACTCTCTCCAGGACACAAAAGGAAAAATTTTTCT
TTCCATCCCTTACATTATTCTCTATTGCAAGTAGAAAGAAATACTGTTTA
ATGGCCTAATAATTTCAAGGAGCCTTGAAAATCATTTCTCTAAATCAAGA
AGGGCAGAAACATTTTACCACTATCATCCCTTCAAATGCATGTTTGTCTC
CTGGAAATCTTTCCCTGTCTCCAGTTGCATACTTGCCCCCACCCATTGAA
GCTCTGAACTCACAACATGCAGTTCCCCATTGCTTATGGTGATCTGTTTC
AAGCCCTCAGAGAAGCAAGATTGATAAACCTGGAAGACAATCACTAACAA
AATAATCATCCCTAATTTACTACTCCCATTTGTTCTTTACTATATTCATC
TCCAGAAAATCATAGATGATCAAAAGATTACCTGGTGCAAAACTCATTTT
ATAAGACAGGAAACTAAGCCTCAAAGAGGTGAGGTACCCAAGGGCATGGG
TAGTTAGTGGCAGATTCACAGTGAAAACAAAGGTGTCCTCTCATTCAGTG
TTCTAGGCACTCTAATGCCCAGTCCAGCATGCACTCCACCTCAACGGTCT
CGGATTGAAACCTGAGTCAGCTGGCTCTGCAGACAAATGCAGAGGAAGAG
CCCTGCCCACTGTGAACACACTCTTACACTTCCAGCTGCTCCTCCAGGAG
CCACAGGCTTCCAGCTCAAGCAAACGTCTGGGAAATCAGCCATACTCAAG
GCTGCACACACCCAGCTCCAACCACCATGTCAAAATGATCCTTTTAACAC
TTCTGTGAAAACCCATTTTGTTTCCCCATTTTATTTTAAAGCATTTCAAA
GCATAGCACACAGGCTGAATTACATAGGCACAGGAGGCAAGACTAGGGAA
AACAGCTAGAGTCAGCTTCTCCCCCTGCAACTTCAATCAAAATAGTTCAC
AGTAAGCACATTCTCCCCTCCTTCTTCCTTGCCAGAAAGCAACAACAAGG
AATCCTCCACTCCAGGGTGATCCTGAATAGGCTATAGCCTCATCACACTA
AGTTCAAAAGGATCAGAATGCAGAGCCAAGGCACTAGACAACTCAGTAGG
GTTATTGAGATGCAGGTAGGTTCTAAGAAGAATATAGAAAATTCTGAACT
TCTCAAATAACATTACTCATACTGTCAATTAATCAAAATGTTGAACAGCT
ATTACTTACTAGACACTATGCTAAGTGCTACAAAGACCAATAAGACTACT
TGTCTTCAGGTAGCTTGTAGTCTAGCAAGAAATGAGCTACAAAGAAAACA
AGAAAGTCAGTATGTAAATGAATGGCATGCAATACACTAGAAATATGCAT
GAACAGCCCTGTATCAGAGAAGAACAAGCAACACAAAAGCAGTCTGTGAA
GACCCATGTTGTATAAAGGGCCACATTAGGCACCATAGGAGTCACAAAGT
TGAAGAACATACTAGCCTTGAAGGCTGACAGTAAAATCTAAACATGGAAA
TAAACACATATCTTACCCATAGCTTTTAAAGCCAAATGAAACACATTACA
AAATGAAAGAAAAGTTACTAGAGAGGTAGGTGTAAGCTACATGTGATGGA
TGGAACTATTTATTTCAACTGAGGTAATCAGGCCAGACCTGTGAGATACA
TTTAAACAGGATTTTGAAGGACAAGTAGCATTTTGATAACCTGAGGATGG
GGAAGGGCATTTCAGAAACAATACCTTAACAAAGGGTGGGTAAGCTTTGA
AAGAAGGCCTGGAGAAAAATCAGGACCCCATCATCCCAGGTCCTGGAGCA
TGGTGGGGGGTAAGGCTAGAAAGGAAGTTGTACGAACTCAAATATTAATT
CAAATGCTAAGAGGCTTACCCTTCATTCTGTATGCAAGCTAATGGCAGAA
GAAAAGGCACAATTAGAGCCATGCTTTGAGAATCATTATTGAAAGCATGT
CGAAGATGGTCTGAAGGAAGCAATTGGGAAAAGCAAGCATAGCTCATCCA
AGTGGGTGAGAGTGTGAGTTAGAGGAAGCTTGGAAATTGGTGATGTGAGA
GATGCTGCAGCTTCTGGGATTGCTGCCTGGTCGTGTGTAGAGGAGGGGCA
GTAGGGCTCATTCTGAATCTTGTCTTGAAAAGCACATAGATAGTGATGCC
AAAACCAGGACTACGGAAATCACTTGAAGCTGTATCCTACCTCCTCCTCC
ATCTGTATCTGCTTCACCTATCAAGGATATCTACTATTGCCACTAAAATT
CAGGTGCTTATGGCCTCCCTCATTCATCAGCCAGGGTTTATCTGGCCAGG
AAAGAGAAGCCCCTTCAGGCATTTGCAACAGAGGGAGTTTAAGTCAGAGA
ACTAGTCACCCTGGTAGTTGAGATTGCCATCAGCAAGAAGCTGTTACCAT
TTGAAGGCTGCAGGGACAAAGGGAGTGAGCAGTCCTCTGGGAGACTGAGG
AAGGAAGCTCCTGGCTTCTCCCCACTTTCCACTTTCCACTTCCCACTTTT
ACTCATCTGTCCTCCAATTCCCTTTTGGCTGAGCCTAGCTGAAACCCAGC
TGACAGGGGAGTTTGAGCAAGCAGCCTCCAGGGTCAGCCCTCTGAGTTAC
AGGTAGAGCAGGACAGGGAGGAATGGATCTCAGGACAAACAGGTTCAGGA
TCCGGCAAACTAATTTTACACTCCAGCCATTGAGTTGGAACTACTGGCCA
GCCTCCCCCCGAGTTAGCATGTAGAATATGGGATACCAGCTGAGTGCCTG
AGAGTTATCATTCACCTCAGTGGGGGTAGGGGCGGAGAAGGGTGACATAT
AGCCAGCCACATCTATATCCACTGGCCCTTCCTTGTCCTAGTCCTCTGTA
TTCCTGAGAAGGAACTATTCTCAAGGACCTGAGTCCAAGTTCATCTTACT
TAGAGTACAGAGAAAAGAACCGCTAACTCCTTCTCTCTTTCCCCCATCAT
GTCTCCTCTCCTTTCTAGTTCTCATCACTCTCTACTCCCCCCTGCCCCCC
TTTTCTCCATCTCCATCACCTTTGTCCAGTCTCTATCCCCATTTTCAATT
CCTTCCTCAAAACACCAAGTTGCTTGGTAGCATGCAGGGTTGGAACATGC
CTTTAACAGAAAATCTCGTGTCTCTTGAACCTAGTTATTTATTCCTTGAG
CAGAGTAGATATTCAACAAAAGAATTGTTAAATTCAATTAAATAGGATAT
ATCTTATTATTAAATATTTTTTTCATTTTTTGTTTACTTATATGATGGGA
ACTTGAGTAGTTTCCGGAATTGTCTCCACAACACCTGGCCAAGGAATCTG
TGAGGAAAAGAAAGATCAAATGGAAAATCAAGGTACATGACACCAGAAGA
CCTACATGTTACTTCAAACTTTTTCTTCCTCATGAACCATTAAAATAGAG
CATAACTCTTCTGGCAGCTGTACATATGTTCATAAATACATGATATTGAC
CCATAGCATAGCAGCTCTGCTCAGCTTCTAACAAGTAAGAATGAAAAGAG
GACATGGTCTTTAGGAACATGAATTTCTGCCCTTCCCATTTTCCTTCAGA
AGGAGAGATTCTTCTATGACCTCATTGGGGGCGGAAATTTTAACCAAAAT
GGTGTCACCCCTGAACCCACTGCGACACGCCACGTAAGTGACCACAGAAG
GAGAAAAGCCCTATAAAAAGAGAGACGATAGCGCTACATTTTGTCCATCT
CATAGCAGGCACAAACTCATCCATCCCCAGTTGATTGGAAGAAACAACGA
TG

11) MMP2. Matrix metalloproteinase-2 (MMP-2) is also known as 72 kDa type IV collagenase and gelatinase A is an enzyme that in humans is encoded by the MMP2 gene (Devarajan et al, 1992; J. Biol. Chem. 267 (35): 25228-32). The matrix metalloproteinase (MMP) family are involved in the breakdown of extracellular matrix in normal physiological processes, such as embryonic development, reproduction, and tissue remodeling, as well as in disease processes, such as arthritis and metastasis. Most MMPs are secreted as inactive proproteins which are activated when cleaved by extracellular proteinases. This gene encodes an enzyme which degrades type IV collagen, the major structural component of basement membranes. The enzyme plays a role in endometrial menstrual breakdown, regulation of vascularization and the inflammatory response. Mutations in the MMP2 gene are associated with Torg-Winchester syndrome, multicentric osteolysis and arthritis syndrome (Martignetti et al., 2001, Nat. Genet. 28 (3): 261-5).

Protein: MMP2 Gene: MMP2 (Homo sapiens, chromosome 16, 55513081-55540586 [NCBI Reference Sequence: NC_{—}000016.9]; start site location: 55513392; strand: positive)

Gene Identification

| | |
|---|---|
| GeneID | 4313 |
| HGNC | 7166 |
| HPRD | 00386 |
| MIM | 120360 |

Targeted Sequences

| | | Relative |
|---|---|---|
| | | upstream |
| | | location |
| Sequence | | to gene |
| ID No: | Sequence (5′-3′) | start site |
| 2666 | GCTCCCTGGCCCCGCGCGTCGC | 9 |
| 2732 | CCGCGGCGCAGGGCTGCGCTCCGAG | 85 |
| 2865 | GCCGCCTGCTACTCCTGGCCTC | 453 |
| 2869 | GCGCACTCGGGCCCGCCCCTCTCTGCCC | 361 |
| 2891 | CGCTCCGAGGGTCCGCTGGCTCGG | 101 |
| 3024 | GTCCACCCTCAGTGCACGACCTCGT | 478 |
| 3066 | CACCGCCTGAGGAAGTCTGGATGC | 239 |
| 3101 | TGCCTCTCTCGCGATCTGGGCG | 512 |
| 3131 | GAGGGACGCCGGCTTGGCTAGGAC | 618 |

Target Shift Sequences

| | | Relative |
|---|---|---|
| | | upstream |
| | | location |
| Sequence | | to gene |
| ID No: | Sequence (5′-3′) | start site |
| 2666 | GCTCCCTGGCCCCGCGCGTCGC | 7 |
| 2667 | CTCCCTGGCCCCGCGCGTCG | 8 |
| 2668 | TCCCTGGCCCCGCGCGTCGC | 9 |
| 2669 | CCCTGGCCCCGCGCGTCGCC | 10 |
| 2670 | CCTGGCCCCGCGCGTCGCCC | 11 |
| 2671 | CTGGCCCCGCGCGTCGCCCG | 12 |
| 2672 | TGGCCCCGCGCGTCGCCCGG | 13 |
| 2673 | GGCCCCGCGCGTCGCCCGGG | 14 |
| 2674 | GCCCCGCGCGTCGCCCGGGG | 15 |
| 2675 | CCCCGCGCGTCGCCCGGGGG | 16 |
| 2676 | CCCGCGCGTCGCCCGGGGGT | 17 |
| 2677 | CCGCGCGTCGCCCGGGGGTC | 18 |
| 2678 | CGCGCGTCGCCCGGGGGTCG | 19 |
| 2679 | GCGCGTCGCCCGGGGGTCGC | 20 |
| 2680 | CGCGTCGCCCGGGGGTCGCT | 21 |
| 2681 | GCGTCGCCCGGGGGTCGCTG | 22 |
| 2682 | CGTCGCCCGGGGGTCGCTGG | 23 |
| 2683 | GTCGCCCGGGGGTCGCTGGC | 24 |
| 2684 | TCGCCCGGGGGTCGCTGGCT | 25 |
| 2685 | CGCCCGGGGGTCGCTGGCTC | 26 |
| 2686 | GCCCGGGGGTCGCTGGCTCG | 27 |
| 2687 | CCCGGGGGTCGCTGGCTCGG | 28 |
| 2688 | CCGGGGGTCGCTGGCTCGGT | 29 |
| 2689 | CGGGGGTCGCTGGCTCGGTG | 30 |
| 2690 | GGGGGTCGCTGGCTCGGTGC | 31 |
| 2691 | GGGGTCGCTGGCTCGGTGCG | 32 |
| 2692 | GGGTCGCTGGCTCGGTGCGT | 33 |
| 2693 | GGTCGCTGGCTCGGTGCGTG | 34 |
| 2694 | GTCGCTGGCTCGGTGCGTGT | 35 |
| 2695 | TCGCTGGCTCGGTGCGTGTG | 36 |
| 2696 | CGCTGGCTCGGTGCGTGTGG | 37 |
| 2697 | GCTGGCTCGGTGCGTGTGGC | 38 |
| 2698 | CTGGCTCGGTGCGTGTGGCC | 39 |
| 2699 | TGGCTCGGTGCGTGTGGCCG | 40 |
| 2700 | GGCTCGGTGCGTGTGGCCGC | 41 |
| 2701 | GCTCGGTGCGTGTGGCCGCC | 42 |
| 2702 | CTCGGTGCGTGTGGCCGCCT | 43 |
| 2703 | TCGGTGCGTGTGGCCGCCTC | 44 |
| 2704 | CGGTGCGTGTGGCCGCCTCG | 45 |
| 2705 | GGTGCGTGTGGCCGCCTCGC | 46 |
| 2706 | GTGCGTGTGGCCGCCTCGCC | 47 |
| 2707 | TGCGTGTGGCCGCCTCGCCG | 48 |
| 2708 | GCGTGTGGCCGCCTCGCCGC | 49 |
| 2709 | CGTGTGGCCGCCTCGCCGCC | 50 |
| 2710 | GTGTGGCCGCCTCGCCGCCT | 51 |
| 2711 | TGTGGCCGCCTCGCCGCCTG | 52 |
| 2712 | GTGGCCGCCTCGCCGCCTGG | 53 |
| 2713 | TGGCCGCCTCGCCGCCTGGT | 54 |
| 2714 | GGCCGCCTCGCCGCCTGGTT | 55 |
| 2715 | GCCGCCTCGCCGCCTGGTTG | 56 |
| 2716 | CCGCCTCGCCGCCTGGTTGG | 57 |
| 2717 | CGCCTCGCCGCCTGGTTGGA | 58 |
| 2718 | GCCTCGCCGCCTGGTTGGAG | 59 |
| 2719 | CCTCGCCGCCTGGTTGGAGC | 60 |
| 2720 | CTCGCCGCCTGGTTGGAGCC | 61 |
| 2721 | TCGCCGCCTGGTTGGAGCCT | 62 |
| 2722 | CGCCGCCTGGTTGGAGCCTG | 63 |
| 2723 | GCCGCCTGGTTGGAGCCTGC | 64 |
| 2724 | CCGCCTGGTTGGAGCCTGCT | 65 |
| 2725 | CGCCTGGTTGGAGCCTGCTC | 66 |
| 2726 | CGCTCCCTGGCCCCGCGCGT | 6 |
| 2727 | GCGCTCCCTGGCCCCGCGCG | 5 |
| 2728 | AGCGCTCCCTGGCCCCGCGC | 4 |
| 2729 | TAGCGCTCCCTGGCCCCGCG | 3 |
| 2730 | GTAGCGCTCCCTGGCCCCGC | 2 |
| 2731 | CGTAGCGCTCCCTGGCCCCG | 1 |
| 2732 | CCGCGGCGCAGGGCTGCGCTCCGAG | 85 |
| 2733 | CGCGGCGCAGGGCTGCGCTC | 86 |
| 2734 | GCGGCGCAGGGCTGCGCTCC | 87 |
| 2735 | CGGCGCAGGGCTGCGCTCCG | 88 |
| 2736 | GGCGCAGGGCTGCGCTCCGA | 89 |
| 2737 | GCGCAGGGCTGCGCTCCGAG | 90 |
| 2738 | CGCAGGGCTGCGCTCCGAGG | 91 |
| 2739 | GCAGGGCTGCGCTCCGAGGG | 92 |
| 2740 | CAGGGCTGCGCTCCGAGGGT | 93 |
| 2741 | AGGGCTGCGCTCCGAGGGTC | 94 |
| 2742 | GGGCTGCGCTCCGAGGGTCC | 95 |
| 2743 | GGCTGCGCTCCGAGGGTCCG | 96 |
| 2744 | GCTGCGCTCCGAGGGTCCGC | 97 |
| 2745 | CTGCGCTCCGAGGGTCCGCT | 98 |
| 2746 | TGCGCTCCGAGGGTCCGCTG | 99 |
| 2747 | GCGCTCCGAGGGTCCGCTGG | 100 |
| 2748 | CGCTCCGAGGGTCCGCTGGC | 101 |
| 2749 | GCTCCGAGGGTCCGCTGGCT | 102 |
| 2750 | CTCCGAGGGTCCGCTGGCTC | 103 |
| 2751 | TCCGAGGGTCCGCTGGCTCG | 104 |
| 2752 | CCGAGGGTCCGCTGGCTCGG | 105 |
| 2753 | CGAGGGTCCGCTGGCTCGGT | 106 |
| 2754 | GAGGGTCCGCTGGCTCGGTG | 107 |
| 2755 | AGGGTCCGCTGGCTCGGTGG | 108 |
| 2756 | GGGTCCGCTGGCTCGGTGGC | 109 |
| 2757 | GGTCCGCTGGCTCGGTGGCC | 110 |
| 2758 | GTCCGCTGGCTCGGTGGCCT | 111 |
| 2759 | TCCGCTGGCTCGGTGGCCTG | 112 |
| 2760 | CCGCTGGCTCGGTGGCCTGG | 113 |
| 2761 | CGCTGGCTCGGTGGCCTGGG | 114 |
| 2762 | GCTGGCTCGGTGGCCTGGGG | 115 |
| 2763 | CTGGCTCGGTGGCCTGGGGT | 116 |
| 2764 | TGGCTCGGTGGCCTGGGGTT | 117 |
| 2765 | GGCTCGGTGGCCTGGGGTTT | 118 |
| 2766 | GCTCGGTGGCCTGGGGTTTG | 119 |
| 2767 | CTCGGTGGCCTGGGGTTTGC | 120 |
| 2768 | TCGGTGGCCTGGGGTTTGCC | 121 |
| 2769 | CGGTGGCCTGGGGTTTGCCC | 122 |
| 2770 | GGTGGCCTGGGGTTTGCCCG | 123 |
| 2771 | GTGGCCTGGGGTTTGCCCGG | 124 |
| 2772 | TGGCCTGGGGTTTGCCCGGC | 125 |
| 2773 | GGCCTGGGGTTTGCCCGGCT | 126 |
| 2774 | GCCTGGGGTTTGCCCGGCTC | 127 |
| 2775 | CCTGGGGTTTGCCCGGCTCA | 128 |
| 2776 | CTGGGGTTTGCCCGGCTCAG | 129 |
| 2777 | TGGGGTTTGCCCGGCTCAGC | 130 |
| 2778 | GGGGTTTGCCCGGCTCAGCG | 131 |
| 2779 | GGGTTTGCCCGGCTCAGCGG | 132 |
| 2780 | GGTTTGCCCGGCTCAGCGGC | 133 |
| 2781 | GTTTGCCCGGCTCAGCGGCT | 134 |
| 2782 | TTTGCCCGGCTCAGCGGCTC | 135 |
| 2783 | TTGCCCGGCTCAGCGGCTCA | 136 |
| 2784 | TGCCCGGCTCAGCGGCTCAT | 137 |
| 2785 | GCCCGGCTCAGCGGCTCATG | 138 |
| 2786 | CCCGGCTCAGCGGCTCATGG | 139 |
| 2787 | CCGGCTCAGCGGCTCATGGT | 140 |
| 2788 | CGGCTCAGCGGCTCATGGTC | 141 |
| 2789 | GGCTCAGCGGCTCATGGTCC | 142 |
| 2790 | GCTCAGCGGCTCATGGTCCG | 143 |
| 2791 | CTCAGCGGCTCATGGTCCGG | 144 |
| 2792 | TCAGCGGCTCATGGTCCGGC | 145 |
| 2793 | CAGCGGCTCATGGTCCGGCC | 146 |
| 2794 | AGCGGCTCATGGTCCGGCCC | 147 |
| 2795 | GCGGCTCATGGTCCGGCCCC | 148 |
| 2796 | CGGCTCATGGTCCGGCCCCC | 149 |
| 2797 | GGCTCATGGTCCGGCCCCCG | 150 |
| 2798 | GCTCATGGTCCGGCCCCCGC | 151 |
| 2799 | CTCATGGTCCGGCCCCCGCG | 152 |
| 2800 | TCATGGTCCGGCCCCCGCGC | 153 |
| 2801 | CATGGTCCGGCCCCCGCGCC | 154 |
| 2802 | ATGGTCCGGCCCCCGCGCCC | 155 |
| 2803 | TGGTCCGGCCCCCGCGCCCC | 156 |
| 2804 | GGTCCGGCCCCCGCGCCCCA | 157 |
| 2805 | GTCCGGCCCCCGCGCCCCAG | 158 |
| 2806 | TCCGGCCCCCGCGCCCCAGC | 159 |
| 2807 | CCGGCCCCCGCGCCCCAGCC | 160 |
| 2808 | CGGCCCCCGCGCCCCAGCCC | 161 |
| 2809 | GGCCCCCGCGCCCCAGCCCC | 162 |
| 2810 | GCCCCCGCGCCCCAGCCCCC | 163 |
| 2811 | CCCCCGCGCCCCAGCCCCCG | 164 |
| 2812 | CCCCGCGCCCCAGCCCCCGC | 165 |
| 2813 | CCCGCGCCCCAGCCCCCGCC | 166 |
| 2814 | CCGCGCCCCAGCCCCCGCCG | 167 |
| 2815 | CGCGCCCCAGCCCCCGCCGC | 168 |
| 2816 | GCGCCCCAGCCCCCGCCGCC | 169 |
| 2817 | CGCCCCAGCCCCCGCCGCCG | 170 |
| 2818 | GCCCCAGCCCCCGCCGCCGC | 171 |
| 2819 | CCCCAGCCCCCGCCGCCGCC | 172 |
| 2820 | CCCAGCCCCCGCCGCCGCCG | 173 |
| 2821 | CCAGCCCCCGCCGCCGCCGC | 174 |
| 2822 | CAGCCCCCGCCGCCGCCGCC | 175 |
| 2823 | AGCCCCCGCCGCCGCCGCCG | 176 |
| 2824 | GCCCCCGCCGCCGCCGCCGC | 177 |
| 2825 | CCCCCGCCGCCGCCGCCGCC | 178 |
| 2826 | CCCCGCCGCCGCCGCCGCCG | 179 |
| 2827 | CCCGCCGCCGCCGCCGCCGC | 180 |
| 2828 | CCCCCGCCGCCGCCGCCGCC | 181 |
| 2829 | CCCCGCCGCCGCCGCCGCCG | 182 |
| 2830 | GCCCCCGCCGCCGCCGCCGC | 183 |
| 2831 | CCGCCGCCGCCGCCGCCGCA | 184 |
| 2832 | CGCCGCCGCCGCCGCCGCAG | 185 |
| 2833 | GCCGCCGCCGCCGCCGCAGG | 186 |
| 2834 | CCGCCGCCGCCGCCGCAGGT | 187 |
| 2835 | CGCCGCCGCCGCCGCAGGTC | 188 |
| 2836 | GCCGCCGCCGCCGCAGGTCC | 189 |
| 2837 | CCGCCGCCGCCGCAGGTCCT | 190 |
| 2838 | CGCCGCCGCCGCAGGTCCTG | 191 |
| 2839 | GCCGCCGCCGCAGGTCCTGG | 192 |
| 2840 | CCGCCGCCGCAGGTCCTGGC | 193 |
| 2841 | CGCCGCCGCAGGTCCTGGCA | 194 |
| 2842 | GCCGCCGCAGGTCCTGGCAA | 195 |
| 2843 | CCGCCGCAGGTCCTGGCAAT | 196 |
| 2844 | CGCCGCAGGTCCTGGCAATC | 197 |
| 2845 | GCCGCAGGTCCTGGCAATCC | 198 |
| 2846 | CCGCAGGTCCTGGCAATCCC | 199 |
| 2847 | CGCAGGTCCTGGCAATCCCT | 200 |
| 2848 | TCCGCGGCGCAGGGCTGCGC | 84 |
| 2849 | CTCCGCGGCGCAGGGCTGCG | 83 |
| 2850 | GCTCCGCGGCGCAGGGCTGC | 82 |
| 2851 | TGCTCCGCGGCGCAGGGCTG | 81 |
| 2852 | CTGCTCCGCGGCGCAGGGCT | 80 |
| 2853 | CCTGCTCCGCGGCGCAGGGC | 79 |
| 2854 | GCCTGCTCCGCGGCGCAGGG | 78 |
| 2855 | AGCCTGCTCCGCGGCGCAGG | 77 |
| 2856 | GAGCCTGCTCCGCGGCGCAG | 76 |
| 2857 | GGAGCCTGCTCCGCGGCGCA | 75 |
| 2858 | TGGAGCCTGCTCCGCGGCGC | 74 |
| 2859 | TTGGAGCCTGCTCCGCGGCG | 73 |
| 2860 | GTTGGAGCCTGCTCCGCGGC | 72 |
| 2861 | GGTTGGAGCCTGCTCCGCGG | 71 |
| 2862 | TGGTTGGAGCCTGCTCCGCG | 70 |
| 2863 | CTGGTTGGAGCCTGCTCCGC | 69 |
| 2864 | CCTGGTTGGAGCCTGCTCCG | 68 |
| 2865 | GCCGCCTGCTACTCCTGGCCTC | 453 |
| 2866 | CCGCCTGCTACTCCTGGCCT | 454 |
| 2867 | CGCCTGCTACTCCTGGCCTC | 455 |
| 2868 | GGCCGCCTGCTACTCCTGGC | 452 |
| 2869 | GCGCACTCGGGCCCGCCCCTCTCTGCCC | 361 |
| 2870 | CGCACTCGGGCCCGCCCCTC | 362 |
| 2871 | GCACTCGGGCCCGCCCCTCT | 363 |
| 2872 | CACTCGGGCCCGCCCCTCTC | 364 |
| 2873 | ACTCGGGCCCGCCCCTCTCT | 365 |
| 2874 | CTCGGGCCCGCCCCTCTCTG | 366 |
| 2875 | TCGGGCCCGCCCCTCTCTGC | 367 |
| 2876 | CGGGCCCGCCCCTCTCTGCC | 368 |
| 2877 | GGGCCCGCCCCTCTCTGCCC | 369 |
| 2878 | GGCCCGCCCCTCTCTGCCCC | 370 |
| 2879 | GCCCGCCCCTCTCTGCCCCA | 371 |
| 2880 | CCCGCCCCTCTCTGCCCCAC | 372 |
| 2881 | CCGCCCCTCTCTGCCCCACC | 373 |
| 2882 | CGCCCCTCTCTGCCCCACCC | 374 |
| 2883 | GGCGCACTCGGGCCCGCCCC | 360 |
| 2884 | GGGCGCACTCGGGCCCGCCC | 359 |
| 2885 | GGGGCGCACTCGGGCCCGCC | 358 |
| 2886 | GGGGGCGCACTCGGGCCCGC | 357 |
| 2887 | GGGGGGCGCACTCGGGCCCG | 356 |
| 2888 | CGGGGGGCGCACTCGGGCCC | 355 |
| 2889 | GCGGGGGGCGCACTCGGGCC | 354 |
| 2890 | GGCGGGGGGCGCACTCGGGC | 353 |
| 2891 | CGCTCCGAGGGTCCGCTGGCTCGG | 101 |
| 2892 | GCTCCGAGGGTCCGCTGGCT | 102 |
| 2893 | CTCCGAGGGTCCGCTGGCTC | 103 |
| 2894 | TCCGAGGGTCCGCTGGCTCG | 104 |
| 2895 | CCGAGGGTCCGCTGGCTCGG | 105 |
| 2896 | CGAGGGTCCGCTGGCTCGGT | 106 |
| 2897 | GAGGGTCCGCTGGCTCGGTG | 107 |
| 2898 | AGGGTCCGCTGGCTCGGTGG | 108 |
| 2899 | GGGTCCGCTGGCTCGGTGGC | 109 |
| 2900 | GGTCCGCTGGCTCGGTGGCC | 110 |
| 2901 | GTCCGCTGGCTCGGTGGCCT | 111 |
| 2902 | TCCGCTGGCTCGGTGGCCTG | 112 |
| 2903 | CCGCTGGCTCGGTGGCCTGG | 113 |
| 2904 | CGCTGGCTCGGTGGCCTGGG | 114 |
| 2905 | GCTGGCTCGGTGGCCTGGGG | 115 |
| 2906 | CTGGCTCGGTGGCCTGGGGT | 116 |
| 2907 | TGGCTCGGTGGCCTGGGGTT | 117 |
| 2908 | GGCTCGGTGGCCTGGGGTTT | 118 |
| 2909 | GCTCGGTGGCCTGGGGTTTG | 119 |
| 2910 | CTCGGTGGCCTGGGGTTTGC | 120 |
| 2911 | TCGGTGGCCTGGGGTTTGCC | 121 |
| 2912 | CGGTGGCCTGGGGTTTGCCC | 122 |
| 2913 | GGTGGCCTGGGGTTTGCCCG | 123 |
| 2914 | GTGGCCTGGGGTTTGCCCGG | 124 |
| 2915 | TGGCCTGGGGTTTGCCCGGC | 125 |
| 2916 | GGCCTGGGGTTTGCCCGGCT | 126 |
| 2917 | GCCTGGGGTTTGCCCGGCTC | 127 |
| 2918 | CCTGGGGTTTGCCCGGCTCA | 128 |
| 2919 | CTGGGGTTTGCCCGGCTCAG | 129 |
| 2920 | TGGGGTTTGCCCGGCTCAGC | 130 |
| 2921 | GGGGTTTGCCCGGCTCAGCG | 131 |
| 2922 | GGGTTTGCCCGGCTCAGCGG | 132 |
| 2923 | GGTTTGCCCGGCTCAGCGGC | 133 |
| 2924 | GTTTGCCCGGCTCAGCGGCT | 134 |
| 2925 | TTTGCCCGGCTCAGCGGCTC | 135 |
| 2926 | TTGCCCGGCTCAGCGGCTCA | 136 |
| 2927 | TGCCCGGCTCAGCGGCTCAT | 137 |
| 2928 | GCCCGGCTCAGCGGCTCATG | 138 |
| 2929 | CCCGGCTCAGCGGCTCATGG | 139 |
| 2930 | CCGGCTCAGCGGCTCATGGT | 140 |
| 2931 | CGGCTCAGCGGCTCATGGTC | 141 |
| 2932 | GGCTCAGCGGCTCATGGTCC | 142 |
| 2933 | GCTCAGCGGCTCATGGTCCG | 143 |
| 2934 | CTCAGCGGCTCATGGTCCGG | 144 |
| 2935 | TCAGCGGCTCATGGTCCGGC | 145 |
| 2936 | CAGCGGCTCATGGTCCGGCC | 146 |
| 2937 | AGCGGCTCATGGTCCGGCCC | 147 |
| 2938 | GCGGCTCATGGTCCGGCCCC | 148 |
| 2939 | CGGCTCATGGTCCGGCCCCC | 149 |
| 2940 | GGCTCATGGTCCGGCCCCCG | 150 |
| 2941 | GCTCATGGTCCGGCCCCCGC | 151 |
| 2942 | CTCATGGTCCGGCCCCCGCG | 152 |
| 2943 | TCATGGTCCGGCCCCCGCGC | 153 |
| 2944 | CATGGTCCGGCCCCCGCGCC | 154 |
| 2945 | ATGGTCCGGCCCCCGCGCCC | 155 |
| 2946 | TGGTCCGGCCCCCGCGCCCC | 156 |
| 2947 | GGTCCGGCCCCCGCGCCCCA | 157 |
| 2948 | GTCCGGCCCCCGCGCCCCAG | 158 |
| 2949 | TCCGGCCCCCGCGCCCCAGC | 159 |
| 2950 | CCGGCCCCCGCGCCCCAGCC | 160 |
| 2951 | CGGCCCCCGCGCCCCAGCCC | 161 |
| 2952 | GGCCCCCGCGCCCCAGCCCC | 162 |
| 2953 | GCCCCCGCGCCCCAGCCCCC | 163 |
| 2954 | CCCCCGCGCCCCAGCCCCCG | 164 |
| 2955 | CCCCGCGCCCCAGCCCCCGC | 165 |
| 2956 | CCCGCGCCCCAGCCCCCGCC | 166 |
| 2957 | CCGCGCCCCAGCCCCCGCCG | 167 |
| 2958 | CGCGCCCCAGCCCCCGCCGC | 168 |
| 2959 | GCGCCCCAGCCCCCGCCGCC | 169 |
| 2960 | CGCCCCAGCCCCCGCCGCCG | 170 |
| 2961 | GCCCCAGCCCCCGCCGCCGC | 171 |
| 2962 | CCCCAGCCCCCGCCGCCGCC | 172 |
| 2963 | CCCAGCCCCCGCCGCCGCCG | 173 |
| 2964 | CCAGCCCCCGCCGCCGCCGC | 174 |
| 2965 | CAGCCCCCGCCGCCGCCGCC | 175 |
| 2966 | AGCCCCCGCCGCCGCCGCCG | 176 |
| 2967 | GCCCCCGCCGCCGCCGCCGC | 177 |
| 2968 | CCCCCGCCGCCGCCGCCGCC | 178 |
| 2969 | CCCCGCCGCCGCCGCCGCCG | 179 |
| 2970 | CCCGCCGCCGCCGCCGCCGC | 180 |
| 2971 | CCGCCGCCGCCGCCGCCGCC | 181 |
| 2972 | CGCCGCCGCCGCCGCCGCCG | 182 |
| 2973 | GCCGCCGCCGCCGCCGCCGC | 183 |
| 2974 | CCGCCGCCGCCGCCGCCGCA | 184 |
| 2975 | CGCCGCCGCCGCCGCCGCAG | 185 |
| 2976 | GCCGCCGCCGCCGCCGCAGG | 186 |
| 2977 | CCGCCGCCGCCGCCGCAGGT | 187 |
| 2978 | CGCCGCCGCCGCCGCAGGTC | 188 |
| 2979 | GCCGCCGCCGCCGCAGGTCC | 189 |
| 2980 | CCGCCGCCGCCGCAGGTCCT | 190 |
| 2981 | CGCCGCCGCCGCAGGTCCTG | 191 |
| 2982 | GCCGCCGCCGCAGGTCCTGG | 192 |
| 2983 | CCGCCGCCGCAGGTCCTGGC | 193 |
| 2984 | CGCCGCCGCAGGTCCTGGCA | 194 |
| 2985 | GCCGCCGCAGGTCCTGGCAA | 195 |
| 2986 | CCGCCGCAGGTCCTGGCAAT | 196 |
| 2987 | CGCCGCAGGTCCTGGCAATC | 197 |
| 2988 | GCCGCAGGTCCTGGCAATCC | 198 |
| 2989 | CCGCAGGTCCTGGCAATCCC | 199 |
| 2990 | CGCAGGTCCTGGCAATCCCT | 200 |
| 2991 | GCGCTCCGAGGGTCCGCTGG | 100 |
| 2992 | TGCGCTCCGAGGGTCCGCTG | 99 |
| 2993 | CTGCGCTCCGAGGGTCCGCT | 98 |
| 2994 | GCTGCGCTCCGAGGGTCCGC | 97 |
| 2995 | GGCTGCGCTCCGAGGGTCCG | 96 |
| 2996 | GGGCTGCGCTCCGAGGGTCC | 95 |
| 2997 | AGGGCTGCGCTCCGAGGGTC | 94 |
| 2998 | CAGGGCTGCGCTCCGAGGGT | 93 |
| 2999 | GCAGGGCTGCGCTCCGAGGG | 92 |
| 3000 | CGCAGGGCTGCGCTCCGAGG | 91 |
| 3001 | GCGCAGGGCTGCGCTCCGAG | 90 |
| 3002 | GGCGCAGGGCTGCGCTCCGA | 89 |
| 3003 | CGGCGCAGGGCTGCGCTCCG | 88 |
| 3004 | GCGGCGCAGGGCTGCGCTCC | 87 |
| 3005 | CGCGGCGCAGGGCTGCGCTC | 86 |
| 3006 | CCGCGGCGCAGGGCTGCGCT | 85 |
| 3007 | TCCGCGGCGCAGGGCTGCGC | 84 |
| 3008 | CTCCGCGGCGCAGGGCTGCG | 83 |
| 3009 | GCTCCGCGGCGCAGGGCTGC | 82 |
| 3010 | TGCTCCGCGGCGCAGGGCTG | 81 |
| 3011 | CTGCTCCGCGGCGCAGGGCT | 80 |
| 3012 | CCTGCTCCGCGGCGCAGGGC | 79 |
| 3013 | GCCTGCTCCGCGGCGCAGGG | 78 |
| 3014 | AGCCTGCTCCGCGGCGCAGG | 77 |
| 3015 | GAGCCTGCTCCGCGGCGCAG | 76 |
| 3016 | GGAGCCTGCTCCGCGGCGCA | 75 |
| 3017 | TGGAGCCTGCTCCGCGGCGC | 74 |
| 3018 | TTGGAGCCTGCTCCGCGGCG | 73 |
| 3019 | GTTGGAGCCTGCTCCGCGGC | 72 |
| 3020 | GGTTGGAGCCTGCTCCGCGG | 71 |
| 3021 | TGGTTGGAGCCTGCTCCGCG | 70 |
| 3022 | CTGGTTGGAGCCTGCTCCGC | 69 |
| 3023 | CCTGGTTGGAGCCTGCTCCG | 68 |
| 3024 | GTCCACCCTCAGTGCACGACCTCGT | 478 |
| 3025 | TCCACCCTCAGTGCACGACC | 479 |
| 3026 | CCACCCTCAGTGCACGACCT | 480 |
| 3027 | CACCCTCAGTGCACGACCTC | 481 |
| 3028 | ACCCTCAGTGCACGACCTCG | 482 |
| 3029 | CCCTCAGTGCACGACCTCGT | 483 |
| 3030 | CCTCAGTGCACGACCTCGTC | 484 |
| 3031 | CTCAGTGCACGACCTCGTCA | 485 |
| 3032 | TCAGTGCACGACCTCGTCAC | 486 |
| 3033 | CAGTGCACGACCTCGTCACC | 487 |
| 3034 | AGTGCACGACCTCGTCACCC | 488 |
| 3035 | GTGCACGACCTCGTCACCCC | 489 |
| 3036 | TGCACGACCTCGTCACCCCA | 490 |
| 3037 | GCACGACCTCGTCACCCCAC | 491 |
| 3038 | CACGACCTCGTCACCCCACT | 492 |
| 3039 | ACGACCTCGTCACCCCACTT | 493 |
| 3040 | CGACCTCGTCACCCCACTTG | 494 |
| 3041 | GACCTCGTCACCCCACTTGC | 495 |
| 3042 | ACCTCGTCACCCCACTTGCC | 496 |
| 3043 | CCTCGTCACCCCACTTGCCT | 497 |
| 3044 | CTCGTCACCCCACTTGCCTC | 498 |
| 3045 | TCGTCACCCCACTTGCCTCT | 499 |
| 3046 | CGTCACCCCACTTGCCTCTC | 500 |
| 3047 | CGTCCACCCTCAGTGCACGA | 477 |
| 3048 | ACGTCCACCCTCAGTGCACG | 476 |
| 3049 | TACGTCCACCCTCAGTGCAC | 475 |
| 3050 | CTACGTCCACCCTCAGTGCA | 474 |
| 3051 | TCTACGTCCACCCTCAGTGC | 473 |
| 3052 | CTCTACGTCCACCCTCAGTG | 472 |
| 3053 | CCTCTACGTCCACCCTCAGT | 471 |
| 3054 | GCCTCTACGTCCACCCTCAG | 470 |
| 3055 | GGCCTCTACGTCCACCCTCA | 469 |
| 3056 | TGGCCTCTACGTCCACCCTC | 468 |
| 3057 | CTGGCCTCTACGTCCACCCT | 467 |
| 3058 | CCTGGCCTCTACGTCCACCC | 466 |
| 3059 | TCCTGGCCTCTACGTCCACC | 465 |
| 3060 | CTCCTGGCCTCTACGTCCAC | 464 |
| 3061 | ACTCCTGGCCTCTACGTCCA | 463 |
| 3062 | TACTCCTGGCCTCTACGTCC | 462 |
| 3063 | CTACTCCTGGCCTCTACGTC | 461 |
| 3064 | GCTACTCCTGGCCTCTACGT | 460 |
| 3065 | TGCTACTCCTGGCCTCTACG | 459 |
| 3066 | CACCGCCTGAGGAAGTCTGGATGC | 256 |
| 3067 | ACCGCCTGAGGAAGTCTGGA | 257 |
| 3068 | CCGCCTGAGGAAGTCTGGAT | 258 |
| 3069 | CGCCTGAGGAAGTCTGGATG | 259 |
| 3070 | CCACCGCCTGAGGAAGTCTG | 255 |
| 3071 | GCCACCGCCTGAGGAAGTCT | 254 |
| 3072 | AGCCACCGCCTGAGGAAGTC | 253 |
| 3073 | CAGCCACCGCCTGAGGAAGT | 252 |
| 3074 | CCAGCCACCGCCTGAGGAAG | 251 |
| 3075 | TCCAGCCACCGCCTGAGGAA | 250 |
| 3076 | CTCCAGCCACCGCCTGAGGA | 249 |
| 3077 | CCTCCAGCCACCGCCTGAGG | 248 |
| 3078 | GCCTCCAGCCACCGCCTGAG | 247 |
| 3079 | AGCCTCCAGCCACCGCCTGA | 246 |
| 3080 | CAGCCTCCAGCCACCGCCTG | 245 |
| 3081 | GCAGCCTCCAGCCACCGCCT | 244 |
| 3082 | CGCAGCCTCCAGCCACCGCC | 243 |
| 3083 | GCGCAGCCTCCAGCCACCGC | 242 |
| 3084 | TGCGCAGCCTCCAGCCACCG | 241 |
| 3085 | ATGCGCAGCCTCCAGCCACC | 240 |
| 3086 | GATGCGCAGCCTCCAGCCAC | 239 |
| 3087 | AGATGCGCAGCCTCCAGCCA | 238 |
| 3088 | CAGATGCGCAGCCTCCAGCC | 237 |
| 3089 | CCAGATGCGCAGCCTCCAGC | 236 |
| 3090 | CCCAGATGCGCAGCCTCCAG | 235 |
| 3091 | CCCCAGATGCGCAGCCTCCA | 234 |
| 3092 | GCCCCAGATGCGCAGCCTCC | 233 |
| 3093 | AGCCCCAGATGCGCAGCCTC | 232 |
| 3094 | AAGCCCCAGATGCGCAGCCT | 231 |
| 3095 | AAAGCCCCAGATGCGCAGCC | 230 |
| 3096 | TAAAGCCCCAGATGCGCAGC | 229 |
| 3097 | TTAAAGCCCCAGATGCGCAG | 228 |
| 3098 | TTTAAAGCCCCAGATGCGCA | 227 |
| 3099 | GTTTAAAGCCCCAGATGCGC | 226 |
| 3100 | TGTTTAAAGCCCCAGATGCG | 225 |
| 3101 | TGCCTCTCTCGCGATCTGGGCG | 512 |
| 3102 | GCCTCTCTCGCGATCTGGGC | 513 |
| 3103 | CCTCTCTCGCGATCTGGGCG | 514 |
| 3104 | CTCTCTCGCGATCTGGGCGC | 515 |
| 3105 | TCTCTCGCGATCTGGGCGCA | 516 |
| 3106 | CTCTCGCGATCTGGGCGCAC | 517 |
| 3107 | TCTCGCGATCTGGGCGCACA | 518 |
| 3108 | CTCGCGATCTGGGCGCACAG | 519 |
| 3109 | TCGCGATCTGGGCGCACAGC | 520 |
| 3110 | CGCGATCTGGGCGCACAGCC | 521 |
| 3111 | GCGATCTGGGCGCACAGCCT | 522 |
| 3112 | CGATCTGGGCGCACAGCCTC | 523 |
| 3113 | GATCTGGGCGCACAGCCTCA | 524 |
| 3114 | ATCTGGGCGCACAGCCTCAG | 525 |
| 3115 | TCTGGGCGCACAGCCTCAGA | 526 |
| 3116 | CTGGGCGCACAGCCTCAGAA | 527 |
| 3117 | TGGGCGCACAGCCTCAGAAC | 528 |
| 3118 | GGGCGCACAGCCTCAGAACC | 529 |
| 3119 | GGCGCACAGCCTCAGAACCC | 530 |
| 3120 | GCGCACAGCCTCAGAACCCC | 531 |
| 3121 | CGCACAGCCTCAGAACCCCC | 532 |
| 3122 | TTGCCTCTCTCGCGATCTGG | 511 |
| 3123 | CTTGCCTCTCTCGCGATCTG | 510 |
| 3124 | ACTTGCCTCTCTCGCGATCT | 509 |
| 3125 | CACTTGCCTCTCTCGCGATC | 508 |
| 3126 | CCACTTGCCTCTCTCGCGAT | 507 |
| 3127 | CCCACTTGCCTCTCTCGCGA | 506 |
| 3128 | CCCCACTTGCCTCTCTCGCG | 505 |
| 3129 | ACCCCACTTGCCTCTCTCGC | 504 |
| 3130 | CACCCCACTTGCCTCTCTCG | 503 |
| 3131 | GAGGGACGCCGGCTTGGCTAGGAC | 618 |
| 3132 | AGGGACGCCGGCTTGGCTAG | 619 |
| 3133 | GGGACGCCGGCTTGGCTAGG | 620 |
| 3134 | GGACGCCGGCTTGGCTAGGA | 621 |
| 3135 | GACGCCGGCTTGGCTAGGAC | 622 |
| 3136 | ACGCCGGCTTGGCTAGGACA | 623 |
| 3137 | CGCCGGCTTGGCTAGGACAC | 624 |
| 3138 | GCCGGCTTGGCTAGGACACC | 625 |
| 3139 | CCGGCTTGGCTAGGACACCC | 626 |
| 3140 | CGGCTTGGCTAGGACACCCT | 627 |
| 3141 | GGAGGGACGCCGGCTTGGCT | 617 |
| 3142 | AGGAGGGACGCCGGCTTGGC | 616 |
| 3143 | TAGGAGGGACGCCGGCTTGG | 615 |
| 3144 | CTAGGAGGGACGCCGGCTTG | 614 |
| 3145 | ACTAGGAGGGACGCCGGCTT | 613 |
| 3146 | TACTAGGAGGGACGCCGGCT | 612 |
| 3147 | CTACTAGGAGGGACGCCGGC | 611 |
| 3148 | ACTACTAGGAGGGACGCCGG | 610 |
| 3149 | TACTACTAGGAGGGACGCCG | 609 |
| 3150 | GTACTACTAGGAGGGACGCC | 608 |
| 3151 | GGTACTACTAGGAGGGACGC | 607 |
| 3152 | CGGTACTACTAGGAGGGACG | 606 |
| 3153 | GCGGTACTACTAGGAGGGAC | 605 |

Hot Zones (Relative upstream location to gene start site)
1-1100
1250-3050
3950-4250

### Examples

Genetic Code (5′ Upstream Region)
(SEQ ID NO: 11960)
TAAGCTTGCTTGACGCAGGGTAGTCACAAACCTTCAATTTGCAAAAT
TGCTATCTCTGCACAGCACAGTAGGGCAAAGTGTGAATAAAATGAGGTAA
CCTGTACCTCCAGCTAAAGTCCCAGAATTAACTTTCCTTGGCTCCAGTGG
ATTCACAAGCCGGTATCTGAATCATCACCTCACCAAGATGCCTGGATCTG
CCTTTTAGCCCAGCTTGGGTCACATTGCCACTGTGGAGCCAGGAGGTGGG
TCACATCTGCTGAATTCAGACCTAGAGTTGGGGAGAAGTAGCTTCCCAAT
GGGAAACTAAGGGGCAGCTACTAAAAGTAGGAGGACAGGGTCTTGGGAAG
GCTTACATGGCACACGGCCACTACCTCCCTCAATGCTCTGTCCCCTGCTT
AGTGTCCCGCACTGTAATTTCTGCCTCTTCATCAATAAAACACCACCTTA
ATGCCACTGTACCCCAGCACCAAAAACAGTGTTCATCAAAAACTTCCCGA
ATAAATGACAGAATTCATGTCATATCGCGACGTCTTCTAATCACAGCCTG
CGTAGTTTTCTGGGGCTGCTGTAAAAAAGCACTACAGACTGGGTGGCTTA
CAACAGAAATTTATTCTCTCAGAGCTCTGAGACTAGAAGTCCAAAACCAA
CATGTCAGCAGGGCCACGCTCCCTCTGAAGCCTCTGGGGGAAGAATTCCT
TCTTGTCTCTTCTAGCTTCTGGGTTGCAGGCAACTCCTTGGGTTGCAGGC
ATTGCTCCAATTTCTGCCTCCATGGTCACATGGAGTTCTTCTTGCTGTGT
GTCTCTGTGTCCAAAGTTTTGTCTTCTTATCATGACAACAGGCTTTGGAT
TAGAGCCCACTCATCTTAACTTGATTGTATCTGCAAAGACCCTATTTCCA
TGTGAGGGCACAGTCACAGGCATTGGGACTTGAACATATCTTTTTGGCAA
CACAATTATATCCACTAAACAGCATTTTTGCATCTATATTAAGAATGACT
AAAAGATGCTGCAGTAACAAACATATCCCAAAAGCTCCATGGTTCGTTGC
CTAAGAGTTTGTTTTTCACTTACGAAGTCTGTAGTGGGTCTGGTTGCTTT
CATTTTACGACTTTGCTAGATCAACACAGGGCTCCCAGGGTTACCGTGGC
AGGGGAAGAGAGATATACAGGAGTGCACAGGGGCCCTGGAGCATATTGCA
TGTGCTCACCAACAAGCCCATTATCATCAGATACTGCCTGTTCTCTCCTC
GTCCCCTGGGCCATTCTAGGCCCCAAGTGAGGGTTCTTGGATCTCACACA
AGAAGGAATTTGAGGCAAGTCCATAAAGTGAAAGCAAGTTTATTAAGAAA
GTAAGGGAATAAAAGAATGGCTATTTCATCGGCAGATCAGCCCCAAAAGC
TGCTGGTTGCCCATTTTTATGGTTATTTCTTGATTATATGCTAAACAAGG
GGTAAATTATTCATGCTTCTGCCTTTTAGGCCGTATAGGATAACTTCCTG
ACGTTGCCATGGCATATGTAAACAGTCGTGGCGCTGGTGGGAGTGTGGCA
GTAAGGCTGACCAGAGGTTTTTCTCATCATCATCTTGGTTTTGGTGGGTC
TTGGCCAGCTTCTTTACTGCAACCTGTTTTATCAGCAAGGTCTTTATGAC
CTGTATCTTGTGCCAGCCTCCTATCTCATTTCGTGATTAGGATTGCCTTA
ATTTACTGGTAATGCAGGCCAGCAGGTCTTAGTCTAACCCCTATTCAAGA
TGGAGTTGCTCTGGTTCCAATGCCTCTGACATAGTCATTGCTGGGCACAG
AAGCCTCTCTCTCACTGAGTGCTGCTTCTGCAGCCATTGCCATCTCTGAA
TGGGCACCAAGCCCCACACTGGAAACCGATACCCTTGGAATGCCAAAACC
ACAGAGGCTGTGAAGAGGTACAGTCAAAGCTGCCCTCTGCCAGAGGAGCT
CCAAAGCATGTTATGCAGACTCCAGAGACTTTCTGAAAAGGTTAAAACTC
AAATTGGGCAAGTGTAAATGAACAACACCTAGTGAAGGGAGTCACATACA
AGGCAATACAATAGAGAGTGGTGAGGACTGTGGCAAACCAAAGTATGTGC
CTCATCTAAAGGGAGCAGTCACTACTCAACTTCAGCAAATTATTGCCATA
TGGAAACTGGCATCCAGTATTGCCAGATTTTTGGGAAATTTTTTTAAAAA
AAGAAAACCAGAAAGTCAGATTTTTACATGAAGTTTCCCAAATTTCAAAA
TGCTGTTCAGGCTGGATTTAGCCCACAGGCCACGAGTTTGCAGCCCCTGC
TTTAGTGAGATAACTTTTTCCATTTTCACTCTCAGCTCTCAGCTCTCCAA
CTTGGCTCTCTGGCTATCCACAGGACGTGGACATGAGCCCAGTGGGGCTG
GGCCAGGAGGCAATCCCCCTTCCCAACTGACCTCAGTCTCGCCCTCTCCA
AAACAGCCAAGGTTTGTCACTGGGTCAGGCTGAAGGGCCTGGCTCCCTCC
TGCGGGGCAAGGTCCCTCCCAAGAGGGTCCTTTAAAACTGACTCTGGAAA
GTCAGAGCACACACCCACCAGACAAGCCTGAACTTGTCTGAAGCCCACTG
AGACCCAAGCCGCAGAGACTTTTCTAGCTGTGATGATCAAGACATAATCG
TGACCTCCAATGCCCCCCACAAGTATATTGCTCCTGATTCTTTCAGCCCC
TGACCTTACTTCTCAAACTGTTCCCTGCTGACCCCCAGTCCTATCTGCCC
CCTTCCTAGGCTGGTCCTTACTGACCCCTCCAGCTCCATCCCCTCACCCT
GTGCCCCACCTTTTTCAGATAGAAAAAACTTTCTTCTCCAGTGCCTCTTG
CTGTTTTTCATCTCTGGGCCATTGTCAATGTTCCCTAAAACATTCCCCAT
ATTCCCCACCCAGCACTCCACCTCTTTAGCTCTTCAGGTCTCAGCTCAGA
AGTCACTTCTTCCAGGAAGCCTTCCTTGATTGTCTTTACTAGTTTAGGGG
CTGAAGTCAGGCGTTCCCAACAGCCTGCTGGAGTTCCCCATCACAGCTTA
TCTCTCAACTGTCTTTCCTGAGAGAGGGAGAAGACATTCCTCAGAGACGG
TTGTCACAGGGAGAACTTCAAAATTGGGATTCGACCTGAGAGGCCACATG
GATTCTTGGCTTGGCGCAGGAAAGGATTCAAGAGTGAGTGGGGAATTCGT
GGAACTGAGGGCTCCTCCCCTTTTTAGACCATATAGGGTAAACCTCCCCA
CATTGCCATGGCATTTATAAACTGCCATGGCACTGGTGGGTGCTTCCTTT
AACATGCTAATGCATTATAATTAGCGTAAAATGAGCAGTGAGGATGACCA
GAGGTCGCTTTCTTTGCCATCTTGGTTTTGGCTGGCTTCTTCACTGCATA
CTGTTTTATCAGTGGGGTCTTTGTGACCTCTATCTTATTAAACCAGTCTT
GCCCAATTTCTATCTCATCCTGTGACCGAGAATGCGGACCCTCCTGGGAG
TGCAGCCCAGCAGGTCTCAGCCTCATTTTACCCAGCCCCCTGTTCAAGAT
GGAGTCGCTCTGGTTCCAACGTCTCTAACGCGGGGCCCCTGACTGCTCTA
TTTCCCAAGGTGTATCTAGCATCTCGCACTATACGAGGCCAAGTTAAGGC
TTACACATTTGCAGAAGGAAAGAGGTAAGGAAGCAACCTGGGACCTTCCA
CTGTCTCTGTTTCCATCTCTCTCTTTCCATCTCTGTTCATCCCAGAATCT
CTCTGTCCCTATCCCTAAATATCGAAAATTTCTGTCTCTGACCATCTATC
ATTGTGGCTGATCATCTGTTTCTGACCATTCCTTCCCGTTCCTGACCCCA
GGGAGTGCAGGGTGTCCTAGCCAAGCCGGCGTCCCTCCTAGTAGTACCGC
TGCTCTCTAACCTCAGGACGTCAAGGGCCTAGAGCGACAGATGTTTCCCA
GCAGGGGGTTCTGAGGCTGTGCGCCCAGATCGCGAGAGAGGCAAGTGGGG
TGACGAGGTCGTGCACTGAGGGTGGACGTAGAGGCCAGGAGTAGCAGGCG
GCCGGGGAAAAGAGGTGGAGAAAGGAAAAAAGAGGAGAAAAGTGGAGGAG
GGCGAGTAGGGGGGTGGGGCAGAGAGGGGCGGGCCCGAGTGCGCCCCCCG
CCCCCAGCCCCGCTCTGCCAGCTCCCTCCCAGCCCAGCCGGCTACATCTG
GCGGCTGCCCTCCCTTGTTTCCGCTGCATCCAGACTTCCTCAGGCGGTGG
CTGGAGGCTGCGCATCTGGGGCTTTAAACATACAAAGGGATTGCCAGGAC
CTGCGGCGGCGGCGGCGGCGGCGGGGGCTGGGGCGCGGGGGCCGGACCAT
GAGCCGCTGAGCCGGGCAAACCCCAGGCCACCGAGCCAGCGGACCCTCGG
AGCGCAGCCCTGCGCCGCGGAGCAGGCTCCAACCAGGCGGCGAGGCGGCC
ACACGCACCGAGCCAGCGACCCCCGGGCGACGCGCGGGGCCAGGGAGCGC
TACGATG

12) FAP. Fibroblast activation protein, alpha (FAP) also known as seprase or 170 kDa melanoma membrane-bound gelatinase is a protein that in humans is encoded by the FAP gene. FAP is a homodimeric integral membrane gelatinase belonging to the serine protease family with dipeptidyl peptidase IV (DPPIV)-like fold, featuring an alpha/beta-hydrolase domain and an eight-bladed beta-propeller domain. FAP has been found to be overexpressed in stromal fibroblasts of solid tumors and epithelial cancers, granulation tissue of healing wounds, and malignant cells of bone and soft tissue sarcomas. This protein is thought to be involved in the control of fibroblast growth or epithelial-mesenchymal interactions during development, tissue repair, and epithelial carcinogenesis (reviewed by Chiri and Charugi, Am J Cancer Res 2011; 1(4):482-497). FAP expression is seen on activated stromal fibroblasts of more than 90% of all human carcinomas. Stromal fibroblasts play an important role in the development, growth and metastasis of carcinomas. It has been shown that targeting FAP inhibits stromagenesis and growth of tumor in mice. Sibrotuzumab a monoclonal antibody and small molecules against FAP are being developed (Edosada et al., J. Biol. Chem. 2006: 281, 7437-7444).

Protein: FAP Gene: FAP (Homo sapiens, chromosome 2, 163027200-163100045 [NCBI Reference Sequence: NC_{—}000002.11]; start site location: 163099837; strand: negative)

Gene Identification

| | |
|---|---|
| GeneID | 2191 |
| HGNC | 3590 |
| HPRD | 02674 |
| MIM | 600403 |

Targeted Sequences

| | | Relative upstream |
|---|---|---|
| Sequence | | location |
| ID No: | Sequence (5′-3′) | to gene start site |
| 3154 | CAGAGCGTGGGTCACTGGATCT | 39 |
| 3171 | CACCAACATCTGCTTACGTTGAC | 272 |
| 3177 | TCCACGGACTTTTGAATACCGTGC | 133 |

Target Shift Sequences

| | | Relative upstream |
|---|---|---|
| Sequence | | location |
| ID No: | Sequence (5′-3′) | to gene start site |
| 3154 | CAGAGCGTGGGTCACTGGATCT | 39 |
| 3155 | AGAGCGTGGGTCACTGGATC | 40 |
| 3156 | GAGCGTGGGTCACTGGATCT | 41 |
| 3157 | AGCGTGGGTCACTGGATCTG | 42 |
| 3158 | GCGTGGGTCACTGGATCTGT | 43 |
| 3159 | CGTGGGTCACTGGATCTGTG | 44 |
| 3160 | TCAGAGCGTGGGTCACTGGA | 38 |
| 3161 | TTCAGAGCGTGGGTCACTGG | 37 |
| 3162 | CTTCAGAGCGTGGGTCACTG | 36 |
| 3163 | TCTTCAGAGCGTGGGTCACT | 35 |
| 3164 | GTCTTCAGAGCGTGGGTCAC | 34 |
| 3165 | TGTCTTCAGAGCGTGGGTCA | 33 |
| 3166 | CTGTCTTCAGAGCGTGGGTC | 32 |
| 3167 | TCTGTCTTCAGAGCGTGGGT | 31 |
| 3168 | TTCTGTCTTCAGAGCGTGGG | 30 |
| 3169 | ATTCTGTCTTCAGAGCGTGG | 29 |
| 3170 | AATTCTGTCTTCAGAGCGTG | 28 |
| 3171 | CACCAACATCTGCTTACGTTGAC | 272 |
| 3172 | ACCAACATCTGCTTACGTTG | 273 |
| 3173 | CCAACATCTGCTTACGTTGA | 274 |
| 3174 | CAACATCTGCTTACGTTGAC | 275 |
| 3175 | ACACCAACATCTGCTTACGT | 271 |
| 3176 | TACACCAACATCTGCTTACG | 270 |
| 3177 | TCCACGGACTTTTGAATACCGTGC | 133 |
| 3178 | CCACGGACTTTTGAATACCG | 134 |
| 3179 | CACGGACTTTTGAATACCGT | 135 |
| 3180 | ACGGACTTTTGAATACCGTG | 136 |
| 3181 | CGGACTTTTGAATACCGTGC | 137 |
| 3182 | GGACTTTTGAATACCGTGCC | 138 |
| 3183 | GACTTTTGAATACCGTGCCA | 139 |

Hot Zones (Relative upstream location to gene start site)
1-400

### Examples

Genetic Code (5′ Upstream Region)
(SEQ ID NO: 11961)
TACCACTCAAAAGTTATGGGACTTTGGGGAAGTTATTTAGATTTTGT
GTGCATCCATGTCCTCATCTGTAAAATGAGGATAATAATAGTACGAATGT
TCTGGGGATAAAAGGAGATAGCACGTGCAAGTGCTGAGAAAAAAACGTCA
TGATCAATAAGAGTATTCAATAGACATGAACTAGTAGTAGTAATATTCTC
TAATCTAAAAATGCTAGTGAAAAAACAATATGTGTTAACAAGTTATGTTA
GTCTATAGGTGCTTACACATTCTATTTATACTATTTAAACAGTCTTCTTG
TTGGTTAACCACTTCTAAAAAGATGTAGTATTTCCCTATTTAAATGACAA
TGAAGCACTGATTTATCTTCTGAGTCTTTGTGTCCTCAGCACTCAAAGAA
ATGGGTTGTGTCTCTGTTCTGTTTCTTTTCTAACCCCATTTTGATAGTTA
ACCCTTTGGGTCTCCAAGGAGCACTTGCCCTAATTATGATAGGATAATGA
AACACTGACATCTAATTATAACATTTATGAAGGTAGTAGATGCCAATTAC
AAACTAGGAGGCATGTGGCTTTATATTCTTCCTTATGTAACAATTGTGGT
TTTAGAAAAGAGATCAGATTCAAAAAATAGTTTGAGCTATATCATTTCCC
ACTGATGCTATATTATGCCACTTCTTCCAGATCTATAACTATGTGATAGT
TATTTTGAGCTTTGAAGACCTCAGGACTCTTTCTAGCTTGGAACTCAAAA
ATCTCTTTGACGATGGGTATTTTGTTGAATCCTTTCATTTAAGCAAGTCC
CTGGAGGAAGAGCTTTTAACCCAGGCACTATATAAATAATCATCGGATTA
ATAGACCCCGATTAAAAAAAAACTGTAAACAAATTAATATTTTGAACACA
GTCCTTGTAGAGTGAAATTGTGTTCTTTTGAGATATGTGTAACAAAGTAC
TTTGAGGATGTGAACATCATTAATATTTTAGCCTTAATTATTTTACCCTC
ACAACCTTAAGTGTCCCCCAACAGCAAATCAGTGAAATGTCAATTATAAT
TTTAAAAAAAATTTATCACTTTGGAATAAAACTTTAGGAAGTATCACAAA
GAAGCAATGTAAGGTGGTAACACTGGGTCCTGTTAAAATCTTGGGCAAGT
TATCCAGTTTTTCTAGGCTGTTTCCTAATCATCAAAATGAGTTTTGGTAT
GGATCAGATGAAATAATGCATTAAAATCACTTTGTAGATAACAGTAAACA
ATAAATGTTTATTGAATCTGAGGAATCAAATGGGTAGGATGTTAGGAGCT
GTTAGGCTCTCTAGAAGCAAATTTTTACTTTAAAGAGTATAAAATCAGGC
TTATGTTTACTGCACTTGTATCCTTATTCCCCTTGTAACTTGTCCTTAAA
TAATTTGTCATGGCTTTTGGTTAATAAACACATCTCTCTTTCATCTCCCC
ACCATAAAATAAAAAGATAACATCCTTATGCTCTCAGCATGGTCTTACCT
TCAGACTCTAGAAATACATAGCTGGATGTGTTTTCTGGGAAAACATATAA
ATTAAAATCATTTTTGGCAGGTAAACATTGGCTACTAATAAATAGTTCTA
GTAAGCTCTCCTCCTTATAACCTAAGGATTTATGTTATAGCTCACTTATC
CAGTGGGCTTACCAGAATGCAGTCATATTCCAAAGTCAGCCTTACAAGGT
CCCTCTCTGGAAAGAACTCAGTTGATGAGCCTCTACTCTCCTAATTGCTG
CCCTTACATTTTTACATGACAAATCATCATCCTTTCTGGTTTTGCAGCAT
TTTAATAGACCCAGAGTTGCTTTCTGAGAAAAGTCTCAGTACTAACAAAG
GTTAGAACTAACAAAGGTTGAACTAAGGATGACATGCATGTGGTATGTGC
CCTTTCCAGTCCTCTTCCCAGGTCCATGAAGAAATCACCAACCAGTGAGA
ACACTCTTTCTACAGAACACAAACTCAGCTTCAGGCTATTTCTGAACAAG
AGCTTTAGGAAGCAAAAGGAAACAAGTATGTAAGAACATTAAGAACATAC
CTGCCATACTTATACTAGAATGTAAGTTCCTTAAGGGCACAGACTGTGTC
TGATTCATTTTTGTTTTCTCAACAAAAATGTTGATGGGGTGTTAACTAAT
TGTATAAAGTAGGAATAAGAGTCAGTTTCGTAAATGTTTTTTAAAAGTTG
ACAAAAGTATTTTCATTTTGATTCCAAAATTAAAAAAAGCTAATAAAGAT
ATTACAATATTTTAAAAATCCAAATTTTATGAGAGTTCTTGTCTGGATGA
AAATTAGAATACATTCACATTATCTCAAACGAATGAACATGTGTGACTTT
ATAAAAACAATACCTCCCTAAACCATGAATTCAGATGGAAAAACTCGACA
TCTTTATTTCTGCAGTCAGTCTCATTTTTCTTAAAACAGTTCAAACTAGT
AAGAATTTTCCAGAAGTTACAGCTTGACTCACCCAACCTTCCAAGGAAAA
AACAAAAAAACTTAAACAGACATTGTTTCACTCTCATCATTTCCCACCCT
TACTAATAGTGGCAACTTAAGTGTATCTTAAAGCACTCCAACCTCTTCAT
AGAGCCTATTAAATGAGTATCTTGTGGACACCCACACACAGTCATAGAAT
CCTAAGTGGTGCTCAGACCAGTCACATGTCAGTGCATTCTTAATTGCTAG
AGCTAACATGCTCTCAGCATGGTCTTTTAATTACACCCTAATAATTTATT
ATAGTTTCTCTCTACAATGTAAAGTCTTGGAAATCACCCACTAAAAAGTG
CCTGTGTACTCTGGGGCTTTGGCAGGCTAGGGCAGAACTTCTGAGAACAC
GGTGTGTTCCAGAGAAGACAATCAATCTGAGAGGACTTACACAGAAACAG
TTCATTCAGGACCTGGCTGCTGGCTTTTATCTGAGATCTGAGGATTTCAC
AATCACTTGGAGATACCTACAAGTGTATAGCACACCTTGGATATTACTCT
TAATGATTACTTCATTTTGTAAAGAGGTGACTCCACCAACAGCAAAGGAG
AGGGCCCAGCCCCAGCCACCAGGAATACAGTTCTCTGCCAGTAAGTGCCT
AATGACTCATTTTCCTCAACAGAATTTTCATAAGGCTGGAATTCAGGGAG
GGATGTCTGGAGAATGTCTGAAAGGAAGTTCACAAGCCACTGTCCTGCTC
TTTGCTGGAGAAAGTGTCCCGTGGTAGCCAGAGAAGTTGACTAAGGCAAA
CAGCAACATGTTTTGGTAACATTTCCCCATTACCTTTCATGTACAATCCA
AGAAAGGTTGCCATGAAGTGTTTTAATCAGGTTGGGAACATTATAAACTT
CGAAAAAAGAAAACCATTAGTGGAAAAATTAAGGACACAGTAGATTTAAC
AACTGTGTTTACGTGGAACCACAAAATCTATCCAAGTGAATTGCATTAAA
ACAGACAGAACACTCCAAGAAACTGTTGTATGTGTATTTTTTTTAATTCA
GTCAACCATTTTACTAATCTGTCAAGATGACCAATTTCTTTGGAATTATG
TAGATTTAGCCAAAATGAAATTATACATAAGATTTACTTTTCTTTTCAGA
TGCTTTTTTATTTATTTTTAAATCTTTATAATTACTAGATGTTCTCCTCT
CTCAGAAGATATTCTGAGAGGAAAGCAAAAATACCACTCTTGTAAAGCCA
TTTCCATTCTTCCAAAGGTCTGCTGGTAAATTATTCTTACTGATCTTTCC
ATCTTTCTAGCCTGTGCATACACACCTAACCCATACTAAATTTCACCAGA
TGGCATTTTATTTCTTTAAAGTAAAGCAGCCGTGGGTTTAGACAGTTGAA
TTTTTAAACTTCTGTATTTACTGAAAGTGCATATGGTGCTATATGGACAA
AGAAATTGTGCTGAAAGAAAAACATTTCTGTCTGCAATACCTCATAATCT
TCCAGAGGAAAAAAAAGTGCAGTTATATGGCACATTTCTCACAAAATCTT
ATGTGGCTTCAATGTTCTTCCTCTGTTAAAAAGTAGATATATGTTTAATG
TACAGACCTGCAAGTTTCATTATTTTAAATTCATCTTTTAGTGGCAAATA
AAAATGTTATGCAAAACCCAATGACTTGCTAAAGTGATCCTTCAGTGAAT
TCTAGAAGAAAATGCAACATAAACCTGAACTGGTAAAAAAGAAAAAATAA
AAACCTCTGTATGTCAACGTAAGCAGATGTTGGTGTAGTTACAAGGATGA
GAAGGCTATAAAACTTCCCTTGAGTCACTCACAGTTCATTTGAGGGCCAA
GAACGCCCCCAAAATCTGTTTCTAATTTTACAGAAATCTTTTGAAACTTG
GCACGGTATTCAAAAGTCCGTGGAAAGAAAAAAACCTTGTCCTGGCTTCA
GCTTCCAACTACAAAGACAGACTTGGTCCTTTTCAACGGTTTTCACAGAT
CCAGTGACCCACGCTCTGAAGACAGAATTAGCTAACTTTCAAAAACATCT
GGAAAAATG

13) P-Selectin. P-selectin is a protein that in humans is encoded by the SELP gene. P-selectin functions as a cell adhesion molecule (CAM) on the surfaces of activated endothelial cells that line the inner surface of blood vessels and activated platelets. In unactivated endothelial cells, it is stored in granules called Weibel-Palade bodies, and α-granules in unactivated platelets (McEver et al., 1989, J. Clin. Invest. 84 (1): 92-9). P-selectin is located on chromosome 1q21-q24, spans>50 kb and contains 17 exons in human. P-selectin is constitutively expressed on megakaryocytes (the precursor of platelets) and endothelial cells (Pan and McEver, 1998; J. Biol. Chem. 273 (16): 10058-67). The expression of P-selectin consists of two distinct mechanisms. One involves P-selectin synthesis by megakaryocytes and endothelial cells, and sorted into membranes of secretory granules until they are activated by agonists such as thrombin and translocated to the plasma membrane from granules. Second, an increased level of mRNA and protein is induced by inflammatory mediators such as tumor necrosis factor-a (TNF-a), LPS, interleukin-4 (IL-4) while TNF-alpha. Selectin-neutralizing monoclonal antibodies, recombinant soluble P-selectin glycoprotein ligand 1 and small-molecule inhibitors of selectins have been tested in clinical trials on patients with multiple trauma, cardiac indications and pediatricasthma, respectively (reviewed in Ley, 2003; Trends Mol. Med, 9 (6): 263-267).

Protein: P-selectin Gene: SELP (Homo sapiens, chromosome 1, 169558087-169599377 [NCBI Reference Sequence: NC_{—}000001.10]; start site location: 169599312; strand: negative)

Gene Identification

| | |
|---|---|
| GeneID | 6403 |
| HGNC | 10721 |
| HPRD | 01433 |
| MIM | 173610 |

Targeted Sequences

| | | Relative |
|---|---|---|
| | | upstream |
| Sequence | | location to gene |
| ID | Sequence (5′-3′) | start site |
| 3184 | TAGCTACGAATAAAGAAATTTGTAG | 2694 |

Hot Zones (Relative upstream location to gene start site)
1550-1800
2650-2800
3100-3250

### Examples

Genetic Code (5′ Upstream Region)
(SEQ ID NO: 11962)
GTCAGGCTGGTCTTGACTCCTGACCTCAGGTGATCCACTCACCTTG
GCCTCCCAAAGCGCTGGGATTATGGCATGAGCCACTGAGTCTGGCTGAAT
GTTAGCTCTCTTGATGCTGTCCCATAAATCTTGTAGGCTTTCATCATTTC
TTTTCATTCTTTTTTCTCCTCTCACTGTATATTTTCAAAAACCTGTCTTC
AGTTCACAGATTCTTTCTTCTGCTTGATCAAGTCTGCTACTGGTGATTTC
TACTGCATTTCTCACTTCATTCATTATATTTTTCAGCTCCAATTTCTTTT
ATGATTTCAATCTTTCTGTTACATTTCTTATGTTGTGCATTTATTGTTTC
TCTGATTTCACCAAATTGTTTCTCTGTGTTTGCTTCAAAGTAACTGAGCT
TCTTTAAAAACAATTATCTTGAATCCATTGTCAGGCCATTTGTAGTACTC
CATTTCTTTTGGGTCAGCTACTGGGAAATTATTGTGTTTCTTAGGTGGTG
ATATTTTAATTTGGGTTTTCATGTTTCTTGCTGCCTTACACTGCTGTCTG
AGCATCTGGTGGATCTGCCCCAATTTCAGGCTGTATGGGCTGACTTTGGT
GGAGAAATACCTTCTTATGTGGAATAATGCGAGGATGCTGGCTGGGTGGG
ATGCAAAAGTTCTGACTTCAGTAGGGGCAAAGCTGTGTGGTCTCCATGCA
GATCTGTCAGCTGAGGTTGGTGTTAGTGAATACTACAGGGATCCTTAGAG
GCCAACACTGTGGGTATCTACAGTGGCAATGAGGCTGTTGAGGTTTTCAA
TTGTGACAAGTCCTCCATATCTCTTTTTTTCCCCACCTGGGAAGTCATGA
CTGAGGACATCCCTCTTGGAATTAGGTCTAACTTGCAGGCCTGCTCCTGG
TGGTGGTGACACTGGTGTCTGATGAACAGTGCCCATGGAGTGGCCAAGAG
CCAAGGCCTGAAGCATGGGCATGCATGGAGGGACCACAGCACCAGATTCA
ATTGTAGCAATGGTACCAGTGCCCAAGGCACAGGCATACTTACTATCACA
TTGATAATGGTGTGTAAAATGCAGGTACTTATAAAGCAGCTAAGGAGCCA
GGGACTTTACTGCATGCATACGCAGAGCTACAGTGGCTCCAGGATCCAGG
GTGTGGGCTAGCTCTCCTTGGTGGCTGAGCTGGTGACTAGAGCATGGACA
GGCACAGAGAAACCTTGACTCTAGGACCCAGGGTGTTCACTAGCTCACTA
TAGTGGTGGCTCTGGTGTTGGAGGTGTGGGTGTGTGTAGTACAGCCTCAG
AGACAGGGTCTGGAGCGCAGGTGTGCACATTACTACAGCAGCTCTGGAGT
TGAGAATATGGGTTACCTTTCTACAGTGGCTGAACTAGTGTCTGGAGCAA
AGACTTTCACAGAGAGAACTTGGCTTGGGGTCCCAGGGTGAGATCTAGTT
CACAACAGCAGTGACTCCAGTGTCTGAGACATGAGGAGGTGCACTGCAGC
CACAGAGCCACAGTCCAGAGTGTGAATATCTGTAGAGCAGCCACAACTTT
TGGGGATCAGGAACACACATAGACTTGTGAGAGGTGGTAACCCTGGCCCC
AGTCCTGGGGCAGTGCAACAATAGCTGCTTCTTGGTGAGGGGGTGTGAGG
GGTAGTGCAACTGTGTTTCCCTTTTTAGCATCCTGCTATGGGAATGGCTG
TTGGATAAAAGATGCCAGTGTCCTCTGTGGAGCAGGACACTGGGGGCCTC
AGTGGCTCTGTGTCACATGACTGACACAGATAGCCTACAAATTTCTTTAT
TCGTAGCTATCTCCTGGTGTCTCATATATGCCAGTCTCACCGGTGATTCT
TCTACATGGATATTCTTTCTTTTCTCCATTGTGTTGTTCCAAATTCTTTA
ACAGGCTCTTGAGCCCCATCCCCCAACTCCCCACCCTTGTGAGGGCTATT
TTGGTTTGTGTATAACTGTCTATGTTTGTTTTTTTGTTGGGGCATAAGGC
TGACATCTCCTACTCCACCATCTTGCTAATGTCACCTGCATAGGAATCTT
TTTATGCTTTCCTTATATTCACTAAAATTTAACAATATCAAACTTAAAAA
CATATGATCAATTGAACTTATTAATATCAAACTTATTATAAATAAGAAAC
TACCAGGCTGGGCATGGTGGCTCATGCCTGTAATCCCAACATTTTGGGAG
GCTGAGGTGAAAGGATCACTTGAGCCCAGGAATTCAAGACCAGCCTGGGA
AATATAGAGAGACCCTATCTCTAGAGATTTTTTTTTTTAATTAGCCAGTA
GTGATGGCACACATCTATAGTCCCAGCTACTCAGGAGGCTGAGGTGGGAG
AATTGCTTGAGCCCAGGAGGTCAAGGCTGGAGCAAGCAGTAATCATGCCA
CTGCACTCCAGCCTGGGCCGCAGAGTGAGACCCTGTCTCAAAAAAAGAAC
CTACTAGTCTACATACCACACTTCCTCATCCCCATCTGAGACTATATATA
TTTTTTCTAACATGAGGCAATGCCAAAAAGAGGGGCTGGTGAGTGAAAGT
AAGAACAGAAAGACATGGAGGCAAGTCTTATAGAATAATAGCCAACACTT
AAACTTACACTTAACAGCGTGATAGGTATTGTTCCAAACACATTAAATTC
ATTTAATGGTCCTTACATGTCTATGTATTTGGTGATTATTATCCTTATTA
TTCACATTGCTGAGTGTATTATTCTGTTCTCATGATGCTGATAGAGACAT
ACCCGAGACTGGATAACTTATTAAAAAAAAAAAGGTTTAATGGACTCACA
GTTCCACGTGGATGGGGAGTCCTCACAATCATGGTAGAAAGCAAAAGACA
CGTCTTACATGGCAGCAGGGAAGAGAGAGAAATGAGAACCAAACAAAAGG
GGTTTCCCCTTATAAAACCATCAGCTCTCATGCGACTTATTCACTACCAT
GAGAACAGTATGGGGGAAACCACCCCCATGATTCAATGATCTACCAGGTG
CCTCCCACAACCTGTGGGAATTATGGGAGCTACAATTCCAGATGAGATTT
GGGTGGGGACACAGCCAAACCACATCACTGAGGAAACTGAGTTATAGGGA
GATTAGTAACGCCCAACACAGCTGGTAGGTGGTGGAGCCAGGCAGTCTGA
CTCTAGGGTCTGGACTCTGAACTGCATCATGCTGCCAAGAAGTTCCTCAT
TTTTTCCTCTCTCTAAGTTTCCCTTATTCCCCTACAGTCATTCCTTCAAC
AGCATTTCCTTCACCATCTTTTCTACTTCTACTATATAATTAATTTTTTC
TTCTTGGTCCCAAATTCCAACGTGCAAATGCAGCCTTATATACCCTAATT
CATCTTTACCTTTAGACTTTCTTCCAATGTTTCTACTTCATTCCATTTTA
AATTTATCCATGAGATGCCTATTTACAAGCTGTAACCATCATGAAGTGAA
TGAAGAATAATACCTACTACTGTACAATAGAATTCCAAGAGTATAAATAG
GAGTTATGGCTTTCTGACTTGAAACTAAATACTTGATACTTGATTTTGCT
GTCTGAGATCAATCTGAAAAGTAATAATAATCACTAACATTTGTTGAGCA
TCAATTGTGGGCCAAGTGTCATTTCAATCACTCTGTACATATTAACTCAT
TTCATCCTACAACAACCCGGTGAGGCAAGTTCTGTTATTCTGTTTTACAG
TTGAGGAAACAGAGGCATAGAGAGCTTAAGTAGTTTGCCCAGTAGATAGC
CAGAAGAGGAGCCAGGATGGGTCTCGGGCAGTTTAACAGCACAGCTGAAG
TCTTAACCACTATGCCAACAGCTTTTTGGTCCTACACATCCCATGGGAAG
AGGAAAATAAAAAGGTATCTATTTGTATACCTTTTTATTTCTGATATAAG
AAGCAGAATTCCTTTCACATGACCTATGTCTATTTAATACGTCATTTTGA
AACTTACCAATAAAATTTCCCAAGCGCCAGAAAACTGTTAGTGGCTTTTT
CCATTTCTCTCTATTTTTTTTTGTGCTACTAATTTTGCTTCTTTCCCTCA
GAAGGCTGCCGGAATAGTAAACATTCACTGACATGTCATAATTACTGGAA
AATGGGCACTGGAAAATCACATTGTAATTAATTCAAAGCATGTTTTCCAA
ATGTACTACTTTAAATTGGAGCTTATATCATAATCCAAGGAAACCTTTGT
GTGTGTACTGTTCCCACATTGCTCAGCCTGGGATATCCAGGAGTAATTCA
CCTTGCGCCTGCCTCCAGACCATCTTCCATGGAAGGGGGTGACCCCTTGC
CTCTTGGCAACCACTATTTCTAAGCTGCCAACATTACTCTTGCATTATCA
ACATTCTAACTTCATGGGAAGGGCTGTGGTGAGTTTCTGGAATGTGAATA
GGAAGTTGTTTTTCTAAACAGCCTGACACTGAGGGGAGGCAGTGAGACTG
TAAGCAGTCTGGGTTGGGCAGAAGGCAGAAAACCAGCAGAGTCACAGAGG
AGATG

14) IL-6. Interleukin 6 (IL-6) acts as both a pro-inflammatory and anti-inflammatory cytokine IL-6 is secreted by T cells and macrophages to stimulate immune response, e.g. during infection and after trauma, especially burns or other tissue damage leading to inflammation. IL-6 also plays a role in fighting infection, as IL-6 has been shown in mice to be required for resistance against bacterium Streptococcus pneumoniae. IL-6 is relevant to many diseases such as diabetes, atherosclerosis, depression, Alzheimer's Disease, systemic lupus erythematosus, multiple myeloma, prostate cancer, behcet's disease,[22] and rheumatoid arthritis (Kishimoto, International Immunology, Vol. 22, No. 5, pp. 347-352). IL-6 is also considered a myokine, a cytokine produced from muscle, and is elevated in response to muscle contraction. It is significantly elevated with exercise, and precedes the appearance of other cytokines in the circulation. During exercise, it is thought to act in a hormone-like manner to mobilize extracellular substrates and/or augment substrate delivery. Additionally, osteoblasts secrete IL-6 to stimulate osteoclast formation. Smooth muscle cells in the tunica media of many blood vessels also produce IL-6 as a pro-inflammatory cytokine IL-6's role as an anti-inflammatory cytokine is mediated through its inhibitory effects on TNF-alpha and IL-1, and activation of IL-1ra and IL-10.

Advanced/metastatic cancer patients have higher levels of IL-6 in their blood. IL-6 is responsible for stimulating acute phase protein synthesis, as well as the production of neutrophils in the bone marrow. It supports the growth of B cells and is antagonistic to regulatory T cells. Therefore there is interest in developing anti-IL-6 agents as therapy against many of these diseases (reviewed in Barton, Expert Opin. Ther. Targets 9 (4): 737-752).

Protein: IL-6 Gene: IL-6 (Homo sapiens, chromosome 7, 22766766-22771621 [NCBI Reference Sequence: NC_{—}000007.13]; start site location: 22766882; strand: positive)

Gene Identification

| | |
|---|---|
| GeneID | 3569 |
| HGNC | 6018 |
| HPRD | 00970 |
| MIM | 147620 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 3185 | | CACCGCGTGGCTTCTGCCACTTTC | 723 |
| 3206 | | TACGGACGCAGGCACGGCTCTAG | 1117 |
| 3226 | | CAGCTCCGCAGCCGTGCACTGTG | 1722 |
| 3255 | | CTTCACCGATTGTCTAAACAGAGAC | 1525 |
| 3256 | IL6_1 | TTCGTTCCCGGTGGGCTCGAGGGC | 35 |
| 3276 | | TGCTTCCGCGTCGGCACCCAAG | 1150 |

Target Shift Sequences

| | | Relative |
|---|---|---|
| | | upstream |
| | | location to |
| Sequence ID | | gene start |
| No: | Sequence (5′-3′) | site |
| 3185 | CACCGCGTGGCTTCTGCCACTTTC | 723 |
| 3186 | ACCGCGTGGCTTCTGCCACT | 724 |
| 3187 | CCGCGTGGCTTCTGCCACTT | 725 |
| 3188 | CGCGTGGCTTCTGCCACTTT | 726 |
| 3189 | GCGTGGCTTCTGCCACTTTC | 727 |
| 3190 | CGTGGCTTCTGCCACTTTCT | 728 |
| 3191 | CCACCGCGTGGCTTCTGCCA | 722 |
| 3192 | GCCACCGCGTGGCTTCTGCC | 721 |
| 3193 | TGCCACCGCGTGGCTTCTGC | 720 |
| 3194 | TTGCCACCGCGTGGCTTCTG | 719 |
| 3195 | TTTGCCACCGCGTGGCTTCT | 718 |
| 3196 | TTTTGCCACCGCGTGGCTTC | 717 |
| 3197 | TTTTTGCCACCGCGTGGCTT | 716 |
| 3198 | CTTTTTGCCACCGCGTGGCT | 715 |
| 3199 | CCTTTTTGCCACCGCGTGGC | 714 |
| 3200 | TCCTTTTTGCCACCGCGTGG | 713 |
| 3201 | CTCCTTTTTGCCACCGCGTG | 712 |
| 3202 | ACTCCTTTTTGCCACCGCGT | 711 |
| 3203 | GACTCCTTTTTGCCACCGCG | 710 |
| 3204 | TGACTCCTTTTTGCCACCGC | 709 |
| 3205 | GTGACTCCTTTTTGCCACCG | 708 |
| 3206 | TACGGACGCAGGCACGGCTCTAG | 1117 |
| 3207 | ACGGACGCAGGCACGGCTCT | 1118 |
| 3208 | CGGACGCAGGCACGGCTCTA | 1119 |
| 3209 | GGACGCAGGCACGGCTCTAG | 1120 |
| 3210 | GACGCAGGCACGGCTCTAGG | 1121 |
| 3211 | ACGCAGGCACGGCTCTAGGC | 1122 |
| 3212 | CGCAGGCACGGCTCTAGGCT | 1123 |
| 3213 | GCAGGCACGGCTCTAGGCTC | 1124 |
| 3214 | CAGGCACGGCTCTAGGCTCT | 1125 |
| 3215 | AGGCACGGCTCTAGGCTCTG | 1126 |
| 3216 | GGCACGGCTCTAGGCTCTGA | 1127 |
| 3217 | GCACGGCTCTAGGCTCTGAA | 1128 |
| 3218 | CACGGCTCTAGGCTCTGAAT | 1129 |
| 3219 | ACGGCTCTAGGCTCTGAATC | 1130 |
| 3220 | CGGCTCTAGGCTCTGAATCT | 1131 |
| 3221 | CTACGGACGCAGGCACGGCT | 1116 |
| 3222 | ACTACGGACGCAGGCACGGC | 1115 |
| 3223 | AACTACGGACGCAGGCACGG | 1114 |
| 3224 | AAACTACGGACGCAGGCACG | 1113 |
| 3225 | GAAACTACGGACGCAGGCAC | 1112 |
| 3226 | CAGCTCCGCAGCCGTGCACTGTG | 1700 |
| 3227 | AGCTCCGCAGCCGTGCACTG | 1701 |
| 3228 | GCTCCGCAGCCGTGCACTGT | 1702 |
| 3229 | CTCCGCAGCCGTGCACTGTG | 1703 |
| 3230 | TCCGCAGCCGTGCACTGTGA | 1704 |
| 3231 | CCGCAGCCGTGCACTGTGAT | 1705 |
| 3232 | CGCAGCCGTGCACTGTGATC | 1706 |
| 3233 | GCAGCCGTGCACTGTGATCC | 1707 |
| 3234 | CAGCCGTGCACTGTGATCCG | 1708 |
| 3235 | AGCCGTGCACTGTGATCCGT | 1709 |
| 3236 | GCCGTGCACTGTGATCCGTC | 1710 |
| 3237 | CCGTGCACTGTGATCCGTCT | 1711 |
| 3238 | CGTGCACTGTGATCCGTCTA | 1712 |
| 3239 | GTGCACTGTGATCCGTCTAT | 1713 |
| 3240 | TGCACTGTGATCCGTCTATG | 1714 |
| 3241 | GCACTGTGATCCGTCTATGT | 1715 |
| 3242 | CACTGTGATCCGTCTATGTA | 1716 |
| 3243 | CCAGCTCCGCAGCCGTGCAC | 1699 |
| 3244 | CCCAGCTCCGCAGCCGTGCA | 1698 |
| 3245 | TCCCAGCTCCGCAGCCGTGC | 1697 |
| 3246 | CTCCCAGCTCCGCAGCCGTG | 1696 |
| 3247 | GCTCCCAGCTCCGCAGCCGT | 1695 |
| 3248 | TGCTCCCAGCTCCGCAGCCG | 1694 |
| 3249 | CTGCTCCCAGCTCCGCAGCC | 1693 |
| 3250 | ACTGCTCCCAGCTCCGCAGC | 1692 |
| 3251 | CACTGCTCCCAGCTCCGCAG | 1691 |
| 3252 | CCACTGCTCCCAGCTCCGCA | 1690 |
| 3253 | GCCACTGCTCCCAGCTCCGC | 1689 |
| 3254 | AGCCACTGCTCCCAGCTCCG | 1688 |
| 3255 | CTTCACCGATTGTCTAAACAGAGAC | 1522 |
| 3256 | TTCGTTCCCGGTGGGCTCGAGGGC | 35 |
| 3257 | TCGTTCCCGGTGGGCTCGAG | 36 |
| 3258 | CGTTCCCGGTGGGCTCGAGG | 37 |
| 3259 | GTTCCCGGTGGGCTCGAGGG | 38 |
| 3260 | TTCCCGGTGGGCTCGAGGGC | 39 |
| 3261 | TCCCGGTGGGCTCGAGGGCA | 40 |
| 3262 | CCCGGTGGGCTCGAGGGCAG | 41 |
| 3263 | CCGGTGGGCTCGAGGGCAGA | 42 |
| 3264 | TTTCGTTCCCGGTGGGCTCG | 34 |
| 3265 | CTTTCGTTCCCGGTGGGCTC | 33 |
| 3266 | TCTTTCGTTCCCGGTGGGCT | 32 |
| 3267 | CTCTTTCGTTCCCGGTGGGC | 31 |
| 3268 | TCTCTTTCGTTCCCGGTGGG | 30 |
| 3269 | TTCTCTTTCGTTCCCGGTGG | 29 |
| 3270 | CTTCTCTTTCGTTCCCGGTG | 28 |
| 3271 | GCTTCTCTTTCGTTCCCGGT | 27 |
| 3272 | AGCTTCTCTTTCGTTCCCGG | 26 |
| 3273 | GAGCTTCTCTTTCGTTCCCG | 25 |
| 3274 | AGAGCTTCTCTTTCGTTCCC | 24 |
| 3275 | TAGAGCTTCTCTTTCGTTCC | 23 |
| 3276 | TGCTTCCGCGTCGGCACCCAAG | 1150 |
| 3277 | GCTTCCGCGTCGGCACCCAA | 1151 |
| 3278 | CTTCCGCGTCGGCACCCAAG | 1152 |
| 3279 | TTCCGCGTCGGCACCCAAGA | 1153 |
| 3280 | TCCGCGTCGGCACCCAAGAA | 1154 |
| 3281 | CCGCGTCGGCACCCAAGAAT | 1155 |
| 3282 | CGCGTCGGCACCCAAGAATT | 1156 |
| 3283 | GCGTCGGCACCCAAGAATTT | 1157 |
| 3284 | CGTCGGCACCCAAGAATTTC | 1158 |
| 3285 | GTCGGCACCCAAGAATTTCT | 1159 |
| 3286 | TCGGCACCCAAGAATTTCTT | 1160 |
| 3287 | CGGCACCCAAGAATTTCTTA | 1161 |
| 3288 | CTGCTTCCGCGTCGGCACCC | 1149 |
| 3289 | TCTGCTTCCGCGTCGGCACC | 1148 |
| 3290 | ATCTGCTTCCGCGTCGGCAC | 1147 |
| 3291 | AATCTGCTTCCGCGTCGGCA | 1146 |
| 3292 | GAATCTGCTTCCGCGTCGGC | 1145 |
| 3293 | TGAATCTGCTTCCGCGTCGG | 1144 |
| 3294 | CTGAATCTGCTTCCGCGTCG | 1143 |
| 3295 | TCTGAATCTGCTTCCGCGTC | 1142 |
| 3296 | CTCTGAATCTGCTTCCGCGT | 1141 |
| 3297 | GCTCTGAATCTGCTTCCGCG | 1140 |
| 3298 | GGCTCTGAATCTGCTTCCGC | 1139 |
| 3299 | AGGCTCTGAATCTGCTTCCG | 1138 |

Hot Zones (Relative upstream location to gene start site)
1-800
1050-1250
1400-1800
2850-3400

### Examples

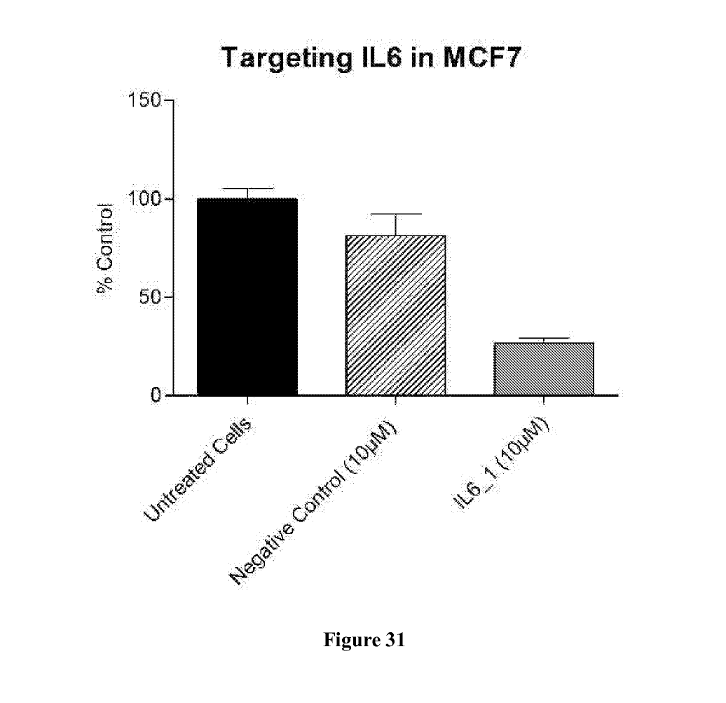

In FIG. 31, In MCF7 (human mammary breast cell line), IL6_{—}1 (145) produced statistically significant (P<0.05) inhibition at 10 μM compared to the untreated and negative control values. The IL6 sequence IL6_{—}1 (145) fits the independent and dependent DNAi motif claims.

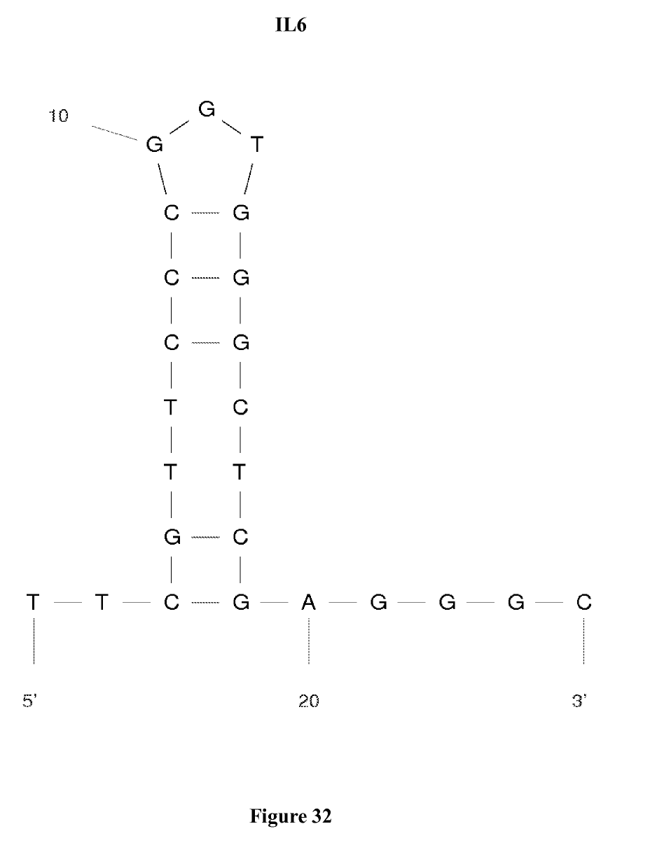

The secondary structure for IL6_{—}1 (145) is shown in FIG. 32.

Genetic Code (5′ Upstream Region)
(SEQ ID NO: 11963)
AGGGACCTCCCCAGCCATGGGGGCAGGGCCAAATGGGGCTTCTTCA
GGACCAGCAAAGCCATTTTTCTCATCAGCAAACTAGCTTCAGAGAAGTTT
GCAATCAGGGCACTCTCTTCCAAGCCTAGAGACCCAGGGAAAGGGGTACG
GGGGTGTCCCAAGGCAAAGAGAATCTACACTTTTTGCCCCCGGAGAGGCT
ACTTCCCTCCCAAGATGCCTGGGATTTTCCACTTCAGCAGGGGGAAGGTA
AGTCACATAGCAAAATAATGAGGGCACAGAACAGATGACCTCCCTATAGA
GTTTTGAATGAGAAACACAGCAGGGCAGATGTGCCCCTTCTCTAGTCTAG
GAGGAGCTAGGTCCAGCCCCTGAACATCCTCCCCCTCAGAAAAGCTGAGG
CCAGACTAAGAATTCACCAGACCAAGGAGCTACAACAGGACATCAGAGCT
GAGGCTGCAAAGCCAGGACTGAGACCAGACCAGGCAGGAAACTGTCAAGA
GCTTTGGTCACCAGGCCTGGCTGCCCTCCAACATCAGCTGGCTCTTTCTA
AATTGACACACCACATGTCCCTAAAATTCTCTCTTCAAGTAATACCACCA
TCAAAGCAGGACATTTCCCAGAGCCTTAGAGCCTGGTGTCTGCTCAGTGG
GACTCAACCCCAGAAGAAGCTGTTAAATCACCCACTGTTTCAGTTTACAA
ACTTCTTACGACTTGGCAACAAGTGAAACTACATTCTGGCAGCAACTGCA
AGTTCCCTAGTACCCAGGACTTCCCGTTTTTTCTTGCTGTACTCCCTCCT
GTTAAATCACAGACTCATCCATCTCCAACCCCCAGAATATAGAGAAAGAG
CACAACACTACATCTTAACTCCTGAGACGTGGAGAACACTTCTCCTCCTG
AGAGCTTAAGTACCAAATGGAAGCTACTTTTCCCCCTTGGTCTCAAATGT
ATTACTAGATTCTGAACTGGACTCCACCATCACGTAAGAAAGCAGTCATG
GGCAGTAATTCTGGGAGATCCAGATAGGACATGCCAGCCCCACACTGGTG
GCATAGGAAGCCAAGTTGCTGCTTCCTCCCTGTGCACTCCCATTTGTCTG
GCCTCTCTTGATCTCAGCTGGCGCTCACTTCACATCAGCTATGATGCAAT
CCAGCAACTAAAGTATTAGTTAATAAATGCTGACAGCACAGCCTTTTCTG
GTCACGTATTCATACTAAAATACGGGGGAGAGTTGGGGGGAGAGGGGGAT
ATATGGGAAATCTCTGTACCTTCCTCTCCATTTTGCTATGACCTAAAGCT
GCCCTTTAAAAAATACAAGGGGCTGGGCACAGTGGTTCACGCCTGTAAAC
CCAGCACTTTGGGAGGCCGAGGCGCGTGGATCACCTGAGGTCAGGAGTTC
AAGACCCGCCTGGCCAACATGGCAAAACCCCGTTTCTACTAAAAATACAA
AAAGTAGCTGGGCGTGGTCGCATGCATCTGTAGTCCCAGCTACTCAGGAG
GCTGAGGCAAGAGAATTGCTTGAACCTGGGAGGCGGCGGTTGAAGTGAGC
CAAGATCATGCCATTGCCCTCCAGCCTGGGCAACAGAGCAAGACTCCTTC
TCAAGAGAAAAAACAAAACAAAACAAGAAAAAACAAAGAATGAGCTCTCC
ACGCGAAAAATCCATTGAGATGCAAAGGAAGGAAGCTATCATTGTGGAAT
TGCACATGTCAGTTACATTAACGTTTTTGGAGCAAGGTAGAGCTCATCTC
TCCCACAAGCAAATTCCAGCCCAAAGCATTGATACTAATAAAGTGCCATG
CTGCGATGTGCAGGGGGCAGACAGTGTCTCCAAGCTCCCTACACACATGC
CTTCCCACAGTTTGCCCTTTCTTGACCCCAGAAGCATCAGGCCCCTTCAC
CCTCGAGGGCCACTATCAGGAGTTTGAATTAATGGCAATCACCATGCACA
GGGAAGGCTGTGGAATTCTGACATAAAAACACTTAGTGGAGGGCTTGGAA
AAAGTCTAGTAGGAGCAAGACGCAAGCTGGACTAATTATCTAAAACAAGA
GACCTGGTTTGGGGATCTTAATGTTCTCAAAAAAGAAAATTATTATTATT
TTTCATTTTGCACTTTGTGCCATAAAACATTTTCAACAAAACATAGAATC
TCATTTCTTTTGAGGGAAAATGATTGGGAGACCAGCTCATTGCTGGCACA
GAGGCCTGGTTCATTCATAATTCCTTCATAGGCAAGACACCAGGTGAACC
GATATAGCCGAGCTGGAAGAGCTCTCCAAGGCAGAGACTCTGAGCCAAGG
AATGTTCAAAGAGCTAGCATGTATTGTGGGATTACTATGCGCCAGGAATT
TTTTACACTGCATCACGTTCCATCTTCACAACAGCCCTAGAAAGGAAGAA
CTATTATTACCCCCGTTTTATAGGTGAATAAACAAGGGCACAGGTCCTTG
ATGTAACAGCCAGGATCAAACAGCTGGGAAGACGAGAAAACCTTTCCCAG
GCTAGGATAACAGAGGATTTGGTTGAAAATACAGGCAATTAGGTGCTACC
TCTGGGAAAAGGGGCCAGGAGAGGAAGGAGACACTTTTCCCTGCATGCCC
TGATGTCCTATTTGAACATTTTATCATGAACACGAACTTCCTATTTAAAA
AACACTTTTTATTGAAAAGATAAATCTGTGTGTTGTATTGTGTCACTCAG
TTCAAGTACTTGAAATTTATTGAATTGTATTTTCTAAAAAATAGATAGTT
GAGTAAAAGCAAGCTCACATTACATAGACGGATCACAGTGCACGGCTGCG
GAGCTGGGAGCAGTGGCTTCGTTTCATGCAGGAAAGAGAACTTGGTTCAG
GAGTGTCTACGTTGCTTAAGACAGGAGAGCACTAAAAATGAAACCATCCA
GCCATCCTCCCCCATTTTCATTTTCACACCAAAGAATCCCACCGCGGCAG
AGGACCACCGTCTCTGTTTAGACAATCGGTGAAGAATGGATGACCTCACT
TTCCCCAACAGGCGGGTCCTGAAATGTTATGCACGAAACAAAACTTGAGT
AAATGCCCAACAGAGGTCACTGTTTTATCGATCTTGAAGAGATCTCTTCT
TAGCAAAGCAAAGAAACCGATTGTGAAGGTAACACCATGTTTGGTAAATA
AGTGTTTTGGTGTTGTGCAAGGGTCTGGTTTCAGCCTGAAGCCATCTCAG
AGCTGTCTGGGTCTCTGGAGACTGGAGGGACAACCTAGTCTAGAGCCCAT
TTGCATGAGACCAAGGATCCTCCTGCAAGAGACACCATCCTGAGGGAAGA
GGGCTTCTGAACCAGCTTGACCCAATAAGAAATTCTTGGGTGCCGACGCG
GAAGCAGATTCAGAGCCTAGAGCCGTGCCTGCGTCCGTAGTTTCCTTCTA
GCTTCTTTTGATTTCAAATCAAGACTTACAGGGAGAGGGAGCGATAAACA
CAAACTCTGCAAGATGCCACAAGGTCCTCCTTTGACATCCCCAACAAAGA
GGTGAGTAGTATTCTCCCCCTTTCTGCCCTGAACCAAGTGGGCTTCAGTA
ATTTCAGGGCTCCAGGAGACCTGGGGCCCATGCAGGTGCCCCAGTGAAAC
AGTGGTGAAGAGACTCAGTGGCAATGGGGAGAGCACTGGCAGCACAAGGC
AAACCTCTGGCACAGAGAGCAAAGTCCTCACTGGGAGGATTCCCAAGGGG
TCACTTGGGAGAGGGCAGGGCAGCAGCCAACCTCCTCTAAGTGGGCTGAA
GCAGGTGAAGAAAGTGGCAGAAGCCACGCGGTGGCAAAAAGGAGTCACAC
ACTCCACCTGGAGACGCCTTGAAGTAACTGCACGAAATTTGAGGATGGCC
AGGCAGTTCTACAACAGCCGCTCACAGGGAGAGCCAGAACACAGAAGAAC
TCAGATGACTGGTAGTATTACCTTCTTCATAATCCCAGGCTTGGGGGGCT
GCGATGGAGTCAGAGGAAACTCAGTTCAGAACATCTTTGGTTTTTACAAA
TACAAATTAACTGGAACGCTAAATTCTAGCCTGTTAATCTGGTCACTGAA
AAAAAATTTTTTTTTTTTCAAAAAACATAGCTTTAGCTTATTTTTTTTCT
CTTTGTAAAACTTCGTGCATGACTTCAGCTTTACTCTTTGTCAAGACATG
CCAAAGTGCTGAGTCACTAATAAAAGAAAAAAAGAAAGTAAAGGAAGAGT
GGTTCTGCTTCTTAGCGCTAGCCTCAATGACGACCTAAGCTGCACTTTTC
CCCCTAGTTGTGTCTTGCCATGCTAAAGGACGTCACATTGCACAATCTTA
ATAAGGTTTCCAATCAGCCCCACCCGCTCTGGCCCCACCCTCACCCTCCA
ACAAAGATTTATCAAATGTGGGATTTTCCCATGAGTCTCAATATTAGAGT
CTCAACCCCCAATAAATATAGGACTGGAGATGTCTGAGGCTCATTCTGCC
CTCGAGCCCACCGGGAACGAAAGAGAAGCTCTATCTCCCCTCCAGGAGCC
CAGCTATG

15) IL-23. IL-23 is produced by dendritic cells and macrophages. Interleukin-23 (IL-23) is a heterodimeric cytokine consisting of two subunits (p40-S-S-p19): p40, a component of the IL-12 cytokine and p19, the product of the IL23 gene (also considered the IL-23 alpha subunit). IL-23 is an important part of the inflammatory response against infection. Both IL-23 and IL-12 can activate the transcription activator STAT4, and stimulate the production of interferon-gamma (IFNG). In contrast to IL-12, which acts mainly on naive CD4(+) T cells, IL-23 preferentially acts on memory CD4(+) T cells (Oppmann et al., 2001, Immunity 13 (5): 715-25).

IL-23 promotes upregulation of the matrix metalloprotease MMP9, increases angiogenesis and reduces CD8+ T-cell infiltration. In conjunction with IL-6 and TGF-β1, IL-23 stimulates naive CD4+ T cells to differentiate into a novel subset of cells called Th17 cells, which are distinct from the classical Th1 and Th2 cells. Th17 cells produce IL-17, a proinflammatory cytokine that enhances T cell priming and stimulates the production of other proinflammatory molecules such as IL-1, IL-6, TNF-alpha, NOS-2, and chemokines resulting in inflammation.

IL-23 may also play a role in the intestinal immune system which has the challenge of maintaining both a state of tolerance toward intestinal antigens and the ability to combat pathogens. This balance is partially achieved by reciprocal regulation of proinflammatory, effector CD4+ T cells and tolerizing, suppressive regulatory T cells. Inflammatory bowel disease (IBD) comprises Crohn's disease (CD) and ulcerative colitis (UC). Genome-wide association studies have linked CD to a number of IL-23 pathway genes, notably IL23R (interleukin 23 receptor). Similar associations in IL-23 pathway genes have been observed in UC. IL23R is a key differentiation feature of CD4+ Th17 cells, effector cells that are critical in mediating antimicrobial defenses. However, IL-23 and Th17 cell dysregulation can lead to end-organ inflammation. The differentiation of inflammatory Th17 cells and suppressive CD4+ Treg subsets is reciprocally regulated by relative concentrations of TGFβ, with the concomitant presence of proinflammatory cytokines favoring Th17 differentiation. The identification of IL-23 pathway and Th17 expressed genes in IBD pathogenesis highlights the importance of the proper regulation of the IL-23/Th17 pathway in maintaining intestinal immune homeostasis (reviewed in Abraham and Cho, 2009; Ann. Rev. Med. 60: 97-110).

Protein: IL23 Gene: IL23A (Homo sapiens, chromosome 12, 56732663-56734194 [NCBI Reference Sequence: NC_{—}000012.11]; start site location: 56732829; strand: positive)

Gene Identification

| | |
|---|---|
| GeneID | 51561 |
| HGNC | 15488 |
| HPRD | 12026 |
| MIM | 605580 |

Targeted Sequences

| | | Relative upstream |
|---|---|---|
| Sequence | | location to |
| ID No: | Sequence (5′-3′) | gene start site |
| 3300 | TCCCTGCATTGTAAGGCCCGCC | 195 |
| 3319 | CACAGCGGGGATGGGGTGGGAGGG | 414 |
| 3320 | GACGTCAGAATGAGGCCATCG | 1296 |
| 3341 | GAGCCAGCACGGTGGTGGGCGCC | 1651 |
| 3365 | GCGTTTGTCCCACCGGCGCCCCG | 4861 |
| 3479 | TAACGCCACCCAACAAGTCCGGCG | 4830 |

Target Shift Sequences

| | | Relative upstream |
|---|---|---|
| Sequence | | location to |
| ID No: | Sequence (5′-3′) | gene start site |
| 3300 | TCCCTGCATTGTAAGGCCCGCC | 195 |
| 3301 | CCCTGCATTGTAAGGCCCGC | 196 |
| 3302 | CCTGCATTGTAAGGCCCGCC | 197 |
| 3303 | CTGCATTGTAAGGCCCGCCC | 198 |
| 3304 | TGCATTGTAAGGCCCGCCCT | 199 |
| 3305 | GCATTGTAAGGCCCGCCCTT | 200 |
| 3306 | CATTGTAAGGCCCGCCCTTT | 201 |
| 3307 | ATTGTAAGGCCCGCCCTTTA | 202 |
| 3308 | TTGTAAGGCCCGCCCTTTAT | 203 |
| 3309 | TGTAAGGCCCGCCCTTTATA | 204 |
| 3310 | GTAAGGCCCGCCCTTTATAC | 205 |
| 3311 | TAAGGCCCGCCCTTTATACC | 206 |
| 3312 | AAGGCCCGCCCTTTATACCA | 207 |
| 3313 | AGGCCCGCCCTTTATACCAG | 208 |
| 3314 | GGCCCGCCCTTTATACCAGC | 209 |
| 3315 | GCCCGCCCTTTATACCAGCA | 210 |
| 3316 | CCCGCCCTTTATACCAGCAG | 211 |
| 3317 | CCGCCCTTTATACCAGCAGG | 212 |
| 3318 | CGCCCTTTATACCAGCAGGT | 213 |
| 3319 | CACAGCGGGGATGGGGTGGGAGGG | 414 |
| 3320 | GACGTCAGAATGAGGCCATCG | 1296 |
| 3321 | ACGTCAGAATGAGGCCATCG | 1297 |
| 3322 | CGTCAGAATGAGGCCATCGG | 1298 |
| 3323 | GTCAGAATGAGGCCATCGGT | 1299 |
| 3324 | TCAGAATGAGGCCATCGGTG | 1300 |
| 3325 | CAGAATGAGGCCATCGGTGA | 1301 |
| 3326 | AGAATGAGGCCATCGGTGAC | 1302 |
| 3327 | GAATGAGGCCATCGGTGACC | 1303 |
| 3328 | AATGAGGCCATCGGTGACCA | 1304 |
| 3329 | ATGAGGCCATCGGTGACCAC | 1305 |
| 3330 | TGAGGCCATCGGTGACCACA | 1306 |
| 3331 | GAGGCCATCGGTGACCACAC | 1307 |
| 3332 | AGGCCATCGGTGACCACACA | 1308 |
| 3333 | GGCCATCGGTGACCACACAG | 1309 |
| 3334 | GCCATCGGTGACCACACAGC | 1310 |
| 3335 | CCATCGGTGACCACACAGCT | 1311 |
| 3336 | CATCGGTGACCACACAGCTG | 1312 |
| 3337 | ATCGGTGACCACACAGCTGG | 1313 |
| 3338 | TCGGTGACCACACAGCTGGC | 1314 |
| 3339 | CGGTGACCACACAGCTGGCT | 1315 |
| 3340 | AGACGTCAGAATGAGGCCAT | 1295 |
| 3341 | GAGCCAGCACGGTGGTGGGCGCC | 1651 |
| 3342 | AGCCAGCACGGTGGTGGGCG | 1652 |
| 3343 | GCCAGCACGGTGGTGGGCGC | 1653 |
| 3344 | CCAGCACGGTGGTGGGCGCC | 1654 |
| 3345 | CAGCACGGTGGTGGGCGCCT | 1655 |
| 3346 | AGCACGGTGGTGGGCGCCTA | 1656 |
| 3347 | GCACGGTGGTGGGCGCCTAT | 1657 |
| 3348 | CACGGTGGTGGGCGCCTATA | 1658 |
| 3349 | ACGGTGGTGGGCGCCTATAG | 1659 |
| 3350 | CGGTGGTGGGCGCCTATAGT | 1660 |
| 3351 | GGTGGTGGGCGCCTATAGTC | 1661 |
| 3352 | GTGGTGGGCGCCTATAGTCC | 1662 |
| 3353 | TGGTGGGCGCCTATAGTCCC | 1663 |
| 3354 | GGTGGGCGCCTATAGTCCCA | 1664 |
| 3355 | GTGGGCGCCTATAGTCCCAG | 1665 |
| 3356 | TGGGCGCCTATAGTCCCAGC | 1666 |
| 3357 | GGGCGCCTATAGTCCCAGCT | 1667 |
| 3358 | GGCGCCTATAGTCCCAGCTA | 1668 |
| 3359 | GCGCCTATAGTCCCAGCTAC | 1669 |
| 3360 | CGCCTATAGTCCCAGCTACT | 1670 |
| 3361 | TGAGCCAGCACGGTGGTGGG | 1650 |
| 3362 | ATGAGCCAGCACGGTGGTGG | 1649 |
| 3363 | AATGAGCCAGCACGGTGGTG | 1648 |
| 3364 | AAATGAGCCAGCACGGTGGT | 1647 |
| 3365 | GCGTTTGTCCCACCGGCGCCCCG | 4861 |
| 3366 | CGTTTGTCCCACCGGCGCCC | 4862 |
| 3367 | GTTTGTCCCACCGGCGCCCC | 4863 |
| 3368 | TTTGTCCCACCGGCGCCCCG | 4864 |
| 3369 | TTGTCCCACCGGCGCCCCGT | 4865 |
| 3370 | TGTCCCACCGGCGCCCCGTA | 4866 |
| 3371 | GTCCCACCGGCGCCCCGTAA | 4867 |
| 3372 | TCCCACCGGCGCCCCGTAAC | 4868 |
| 3373 | CCCACCGGCGCCCCGTAACC | 4869 |
| 3374 | CCACCGGCGCCCCGTAACCT | 4870 |
| 3375 | CACCGGCGCCCCGTAACCTC | 4871 |
| 3376 | ACCGGCGCCCCGTAACCTCT | 4872 |
| 3377 | CCGGCGCCCCGTAACCTCTT | 4873 |
| 3378 | CGGCGCCCCGTAACCTCTTT | 4874 |
| 3379 | GGCGCCCCGTAACCTCTTTT | 4875 |
| 3380 | GCGCCCCGTAACCTCTTTTT | 4876 |
| 3381 | CGCCCCGTAACCTCTTTTTC | 4877 |
| 3382 | GCCCCGTAACCTCTTTTTCC | 4878 |
| 3383 | CCCCGTAACCTCTTTTTCCG | 4879 |
| 3384 | CCCGTAACCTCTTTTTCCGG | 4880 |
| 3385 | CCGTAACCTCTTTTTCCGGC | 4881 |
| 3386 | CGTAACCTCTTTTTCCGGCG | 4882 |
| 3387 | GTAACCTCTTTTTCCGGCGC | 4883 |
| 3388 | TAACCTCTTTTTCCGGCGCG | 4884 |
| 3389 | AACCTCTTTTTCCGGCGCGT | 4885 |
| 3390 | ACCTCTTTTTCCGGCGCGTG | 4886 |
| 3391 | CCTCTTTTTCCGGCGCGTGC | 4887 |
| 3392 | CTCTTTTTCCGGCGCGTGCG | 4888 |
| 3393 | TCTTTTTCCGGCGCGTGCGT | 4889 |
| 3394 | CTTTTTCCGGCGCGTGCGTC | 4890 |
| 3395 | TTTTTCCGGCGCGTGCGTCA | 4891 |
| 3396 | TTTTCCGGCGCGTGCGTCAC | 4892 |
| 3397 | TTTCCGGCGCGTGCGTCACA | 4893 |
| 3398 | TTCCGGCGCGTGCGTCACAC | 4894 |
| 3399 | TCCGGCGCGTGCGTCACACG | 4895 |
| 3400 | CCGGCGCGTGCGTCACACGC | 4896 |
| 3401 | CGGCGCGTGCGTCACACGCT | 4897 |
| 3402 | GGCGCGTGCGTCACACGCTC | 4898 |
| 3403 | GCGCGTGCGTCACACGCTCT | 4899 |
| 3404 | CGCGTGCGTCACACGCTCTC | 4900 |
| 3405 | GCGTGCGTCACACGCTCTCT | 4901 |
| 3406 | CGTGCGTCACACGCTCTCTC | 4902 |
| 3407 | GTGCGTCACACGCTCTCTCC | 4903 |
| 3408 | TGCGTCACACGCTCTCTCCT | 4904 |
| 3409 | GCGTCACACGCTCTCTCCTG | 4905 |
| 3410 | CGTCACACGCTCTCTCCTGG | 4906 |
| 3411 | GTCACACGCTCTCTCCTGGG | 4907 |
| 3412 | TCACACGCTCTCTCCTGGGG | 4908 |
| 3413 | CACACGCTCTCTCCTGGGGT | 4909 |
| 3414 | ACACGCTCTCTCCTGGGGTC | 4910 |
| 3415 | CACGCTCTCTCCTGGGGTCG | 4911 |
| 3416 | ACGCTCTCTCCTGGGGTCGC | 4912 |
| 3417 | CGCTCTCTCCTGGGGTCGCC | 4913 |
| 3418 | GCTCTCTCCTGGGGTCGCCG | 4914 |
| 3419 | CTCTCTCCTGGGGTCGCCGT | 4915 |
| 3420 | TCTCTCCTGGGGTCGCCGTA | 4916 |
| 3421 | CTCTCCTGGGGTCGCCGTAC | 4917 |
| 3422 | TCTCCTGGGGTCGCCGTACC | 4918 |
| 3423 | CTCCTGGGGTCGCCGTACCT | 4919 |
| 3424 | TCCTGGGGTCGCCGTACCTG | 4920 |
| 3425 | CCTGGGGTCGCCGTACCTGG | 4921 |
| 3426 | CTGGGGTCGCCGTACCTGGC | 4922 |
| 3427 | TGGGGTCGCCGTACCTGGCT | 4923 |
| 3428 | GGGGTCGCCGTACCTGGCTC | 4924 |
| 3429 | GGGTCGCCGTACCTGGCTCC | 4925 |
| 3430 | GGTCGCCGTACCTGGCTCCT | 4926 |
| 3431 | GTCGCCGTACCTGGCTCCTT | 4927 |
| 3432 | TCGCCGTACCTGGCTCCTTC | 4928 |
| 3433 | CGCCGTACCTGGCTCCTTCT | 4929 |
| 3434 | GCCGTACCTGGCTCCTTCTG | 4930 |
| 3435 | CCGTACCTGGCTCCTTCTGA | 4931 |
| 3436 | CGTACCTGGCTCCTTCTGAT | 4932 |
| 3437 | TGCGTTTGTCCCACCGGCGC | 4860 |
| 3438 | CTGCGTTTGTCCCACCGGCG | 4859 |
| 3439 | GCTGCGTTTGTCCCACCGGC | 4858 |
| 3440 | GGCTGCGTTTGTCCCACCGG | 4857 |
| 3441 | TGGCTGCGTTTGTCCCACCG | 4856 |
| 3442 | CTGGCTGCGTTTGTCCCACC | 4855 |
| 3443 | TCTGGCTGCGTTTGTCCCAC | 4854 |
| 3444 | GTCTGGCTGCGTTTGTCCCA | 4853 |
| 3445 | CGTCTGGCTGCGTTTGTCCC | 4852 |
| 3446 | GCGTCTGGCTGCGTTTGTCC | 4851 |
| 3447 | GGCGTCTGGCTGCGTTTGTC | 4850 |
| 3448 | CGGCGTCTGGCTGCGTTTGT | 4849 |
| 3449 | CCGGCGTCTGGCTGCGTTTG | 4848 |
| 3450 | TCCGGCGTCTGGCTGCGTTT | 4847 |
| 3451 | GTCCGGCGTCTGGCTGCGTT | 4846 |
| 3452 | AGTCCGGCGTCTGGCTGCGT | 4845 |
| 3453 | AAGTCCGGCGTCTGGCTGCG | 4844 |
| 3454 | CAAGTCCGGCGTCTGGCTGC | 4843 |
| 3455 | ACAAGTCCGGCGTCTGGCTG | 4842 |
| 3456 | AACAAGTCCGGCGTCTGGCT | 4841 |
| 3457 | CAACAAGTCCGGCGTCTGGC | 4840 |
| 3458 | CCAACAAGTCCGGCGTCTGG | 4839 |
| 3459 | CCCAACAAGTCCGGCGTCTG | 4838 |
| 3460 | ACCCAACAAGTCCGGCGTCT | 4837 |
| 3461 | CACCCAACAAGTCCGGCGTC | 4836 |
| 3462 | CCACCCAACAAGTCCGGCGT | 4835 |
| 3463 | GCCACCCAACAAGTCCGGCG | 4834 |
| 3464 | CGCCACCCAACAAGTCCGGC | 4833 |
| 3465 | ACGCCACCCAACAAGTCCGG | 4832 |
| 3466 | AACGCCACCCAACAAGTCCG | 4831 |
| 3467 | TAACGCCACCCAACAAGTCC | 4830 |
| 3468 | CTAACGCCACCCAACAAGTC | 4829 |
| 3469 | TCTAACGCCACCCAACAAGT | 4828 |
| 3470 | TTCTAACGCCACCCAACAAG | 4827 |
| 3471 | TTTCTAACGCCACCCAACAA | 4826 |
| 3472 | CTTTCTAACGCCACCCAACA | 4825 |
| 3473 | ACTTTCTAACGCCACCCAAC | 4824 |
| 3474 | TACTTTCTAACGCCACCCAA | 4823 |
| 3475 | TTACTTTCTAACGCCACCCA | 4822 |
| 3476 | GTTACTTTCTAACGCCACCC | 4821 |
| 3477 | AGTTACTTTCTAACGCCACC | 4820 |
| 3478 | GAGTTACTTTCTAACGCCAC | 4819 |
| 3479 | TAACGCCACCCAACAAGTCCGGCG | 4830 |
| 3480 | AACGCCACCCAACAAGTCCG | 4831 |
| 3481 | ACGCCACCCAACAAGTCCGG | 4832 |
| 3482 | CGCCACCCAACAAGTCCGGC | 4833 |
| 3483 | GCCACCCAACAAGTCCGGCG | 4834 |
| 3484 | CCACCCAACAAGTCCGGCGT | 4835 |
| 3485 | CACCCAACAAGTCCGGCGTC | 4836 |
| 3486 | ACCCAACAAGTCCGGCGTCT | 4837 |
| 3487 | CCCAACAAGTCCGGCGTCTG | 4838 |
| 3488 | CCAACAAGTCCGGCGTCTGG | 4839 |
| 3489 | CAACAAGTCCGGCGTCTGGC | 4840 |
| 3490 | AACAAGTCCGGCGTCTGGCT | 4841 |
| 3491 | ACAAGTCCGGCGTCTGGCTG | 4842 |
| 3492 | CAAGTCCGGCGTCTGGCTGC | 4843 |
| 3493 | AAGTCCGGCGTCTGGCTGCG | 4844 |
| 3494 | AGTCCGGCGTCTGGCTGCGT | 4845 |
| 3495 | GTCCGGCGTCTGGCTGCGTT | 4846 |
| 3496 | TCCGGCGTCTGGCTGCGTTT | 4847 |
| 3497 | CCGGCGTCTGGCTGCGTTTG | 4848 |
| 3498 | CGGCGTCTGGCTGCGTTTGT | 4849 |
| 3499 | GGCGTCTGGCTGCGTTTGTC | 4850 |
| 3500 | GCGTCTGGCTGCGTTTGTCC | 4851 |
| 3501 | CGTCTGGCTGCGTTTGTCCC | 4852 |
| 3502 | GTCTGGCTGCGTTTGTCCCA | 4853 |
| 3503 | TCTGGCTGCGTTTGTCCCAC | 4854 |
| 3504 | CTGGCTGCGTTTGTCCCACC | 4855 |
| 3505 | TGGCTGCGTTTGTCCCACCG | 4856 |
| 3506 | GGCTGCGTTTGTCCCACCGG | 4857 |
| 3507 | GCTGCGTTTGTCCCACCGGC | 4858 |
| 3508 | CTGCGTTTGTCCCACCGGCG | 4859 |
| 3509 | TGCGTTTGTCCCACCGGCGC | 4860 |
| 3510 | GCGTTTGTCCCACCGGCGCC | 4861 |
| 3511 | CGTTTGTCCCACCGGCGCCC | 4862 |
| 3512 | GTTTGTCCCACCGGCGCCCC | 4863 |
| 3513 | TTTGTCCCACCGGCGCCCCG | 4864 |
| 3514 | TTGTCCCACCGGCGCCCCGT | 4865 |
| 3515 | TGTCCCACCGGCGCCCCGTA | 4866 |
| 3516 | GTCCCACCGGCGCCCCGTAA | 4867 |
| 3517 | TCCCACCGGCGCCCCGTAAC | 4868 |
| 3518 | CCCACCGGCGCCCCGTAACC | 4869 |
| 3519 | CCACCGGCGCCCCGTAACCT | 4870 |
| 3520 | CACCGGCGCCCCGTAACCTC | 4871 |
| 3521 | ACCGGCGCCCCGTAACCTCT | 4872 |
| 3522 | CCGGCGCCCCGTAACCTCTT | 4873 |
| 3523 | CGGCGCCCCGTAACCTCTTT | 4874 |
| 3524 | GGCGCCCCGTAACCTCTTTT | 4875 |
| 3525 | GCGCCCCGTAACCTCTTTTT | 4876 |
| 3526 | CGCCCCGTAACCTCTTTTTC | 4877 |
| 3527 | GCCCCGTAACCTCTTTTTCC | 4878 |
| 3528 | CCCCGTAACCTCTTTTTCCG | 4879 |
| 3529 | CCCGTAACCTCTTTTTCCGG | 4880 |
| 3530 | CCGTAACCTCTTTTTCCGGC | 4881 |
| 3531 | CGTAACCTCTTTTTCCGGCG | 4882 |
| 3532 | GTAACCTCTTTTTCCGGCGC | 4883 |
| 3533 | TAACCTCTTTTTCCGGCGCG | 4884 |
| 3534 | AACCTCTTTTTCCGGCGCGT | 4885 |
| 3535 | ACCTCTTTTTCCGGCGCGTG | 4886 |
| 3536 | CCTCTTTTTCCGGCGCGTGC | 4887 |
| 3537 | CTCTTTTTCCGGCGCGTGCG | 4888 |
| 3538 | TCTTTTTCCGGCGCGTGCGT | 4889 |
| 3539 | CTTTTTCCGGCGCGTGCGTC | 4890 |
| 3540 | TTTTTCCGGCGCGTGCGTCA | 4891 |
| 3541 | TTTTCCGGCGCGTGCGTCAC | 4892 |
| 3542 | TTTCCGGCGCGTGCGTCACA | 4893 |
| 3543 | TTCCGGCGCGTGCGTCACAC | 4894 |
| 3544 | TCCGGCGCGTGCGTCACACG | 4895 |
| 3545 | CCGGCGCGTGCGTCACACGC | 4896 |
| 3546 | CGGCGCGTGCGTCACACGCT | 4897 |
| 3547 | GGCGCGTGCGTCACACGCTC | 4898 |
| 3548 | GCGCGTGCGTCACACGCTCT | 4899 |
| 3549 | CGCGTGCGTCACACGCTCTC | 4900 |
| 3550 | GCGTGCGTCACACGCTCTCT | 4901 |
| 3551 | CGTGCGTCACACGCTCTCTC | 4902 |
| 3552 | GTGCGTCACACGCTCTCTCC | 4903 |
| 3553 | TGCGTCACACGCTCTCTCCT | 4904 |
| 3554 | GCGTCACACGCTCTCTCCTG | 4905 |
| 3555 | CGTCACACGCTCTCTCCTGG | 4906 |
| 3556 | GTCACACGCTCTCTCCTGGG | 4907 |
| 3557 | TCACACGCTCTCTCCTGGGG | 4908 |
| 3558 | CACACGCTCTCTCCTGGGGT | 4909 |
| 3559 | ACACGCTCTCTCCTGGGGTC | 4910 |
| 3560 | CACGCTCTCTCCTGGGGTCG | 4911 |
| 3561 | ACGCTCTCTCCTGGGGTCGC | 4912 |
| 3562 | CGCTCTCTCCTGGGGTCGCC | 4913 |
| 3563 | GCTCTCTCCTGGGGTCGCCG | 4914 |
| 3564 | CTCTCTCCTGGGGTCGCCGT | 4915 |
| 3565 | TCTCTCCTGGGGTCGCCGTA | 4916 |
| 3566 | CTCTCCTGGGGTCGCCGTAC | 4917 |
| 3567 | TCTCCTGGGGTCGCCGTACC | 4918 |
| 3568 | CTCCTGGGGTCGCCGTACCT | 4919 |
| 3569 | TCCTGGGGTCGCCGTACCTG | 4920 |
| 3570 | CCTGGGGTCGCCGTACCTGG | 4921 |
| 3571 | CTGGGGTCGCCGTACCTGGC | 4922 |
| 3572 | TGGGGTCGCCGTACCTGGCT | 4923 |
| 3573 | GGGGTCGCCGTACCTGGCTC | 4924 |
| 3574 | GGGTCGCCGTACCTGGCTCC | 4925 |
| 3575 | GGTCGCCGTACCTGGCTCCT | 4926 |
| 3576 | GTCGCCGTACCTGGCTCCTT | 4927 |
| 3577 | TCGCCGTACCTGGCTCCTTC | 4928 |
| 3578 | CGCCGTACCTGGCTCCTTCT | 4929 |
| 3579 | GCCGTACCTGGCTCCTTCTG | 4930 |
| 3580 | CCGTACCTGGCTCCTTCTGA | 4931 |
| 3581 | CGTACCTGGCTCCTTCTGAT | 4932 |
| 3582 | CTAACGCCACCCAACAAGTC | 4829 |
| 3583 | TCTAACGCCACCCAACAAGT | 4828 |
| 3584 | TTCTAACGCCACCCAACAAG | 4827 |
| 3585 | TTTCTAACGCCACCCAACAA | 4826 |
| 3586 | CTTTCTAACGCCACCCAACA | 4825 |
| 3587 | ACTTTCTAACGCCACCCAAC | 4824 |
| 3588 | TACTTTCTAACGCCACCCAA | 4823 |
| 3589 | TTACTTTCTAACGCCACCCA | 4822 |
| 3590 | GTTACTTTCTAACGCCACCC | 4821 |
| 3591 | AGTTACTTTCTAACGCCACC | 4820 |
| 3592 | GAGTTACTTTCTAACGCCAC | 4819 |

```
Hot Zones (Relative upstream location to gene start site)
  1-500
  950-1400
1450-1800
3390-4050
4300-5000
```

### Examples

Genetic Code (5′ Upstream Region)

(SEQ ID NO: 11964)

AACTCCCATCCGTGATTGTTCCCTCCCCAGAGACCCCGGTAACATTCCCG
GGTAACAAGATGCCCCTGGTTATCAAATTCCCCTAGCTCTTGAGGCTGGC
TGGACGTTATCCCTCAGAGGGGGATGAGCATGGCAAATTGGGACTTGTTA
TTCTGAAGGATTCGTGGGTCCTGTGAACTCTAATTACTTTGAAATGGGTC
TAGGTTGTGAGATGTCTCAGAGCACTTTAGCTCAGCTGTTATTACTGTTT
CTAAAGGCCACATAAAGGGACTCTGATGGGAGACATTCCTCATGGAGGAT
TCAATTCTATAACATTTCTCTCAATAAAGGCTGGTAAATAGACCTTCATT
AAAGGAACCAAGAATTTAAATTTCTAGGACTCAGAGGGGTGGGGTCCTAT
ACCCAGTCAGAGATCCTACCTAGAGCCTAGACCAAGAGAAAAACACAGAT
GGTCTCTCAAACTGATTTGATCTGACTTCGCAGGTCATTAGATATAGAAT
CTCCGAAAAAGGTGGATGCTGAGAGACATAGACAGTTCCTACACTTTAAG
AAATCTCCATCTTGAGGTCTCAAATTGAGAAAGACTTAACAGACCCATGA
GAGTTACAGATCCCTAATAACCTGGGCTAAATAATCCATGTCTGCCGGGC
GCAGTGGCTCACGCCTGTAATCCCAGCACTTTGGGAGGCCGAGGTGGGCG
GATCACCTGAGGTCGGGAGTTCGAGACCAACCTGACCAATATGGAGAAAC
CCCGTCTCCACTAAAAATACAAAATTAGCCGGGCCTGGTGACGGGAGCCT
GTAATCCCAGCTACTTGGGAGGCTGAGGCAGGAGAATCATTTGAACCTGG
GAGGCGGAGGTTGCAGTGAGCCGAGATGGCACCATTGCACTCCAGCCTGG
TCAGTAAGAGCGAAACTCCGTCTCAAAAAAAAGAAAAAAAAAAAGAAAAG
AAAAAAGGATACTGTGAGGAGACACAAGAGCATCCATGACATAGATTATT
TAGCTCAGCTGTAATTACTGTTTCTAATACAGTAATATTAGATGGTGATC
TGCCTGCCTCGGCCTCCCAAAGTTCTGGGATTACAGGTGTGAGCCACCGC
GCCCAGCCTTTTTTTTTTTTTTTTTTTGAGACAGGATCTCACTCTGTTGC
CCATGCTTAAGCGCATTGGCCCTCTCACTCACTGTAGCCTCAACCTCCTG
GGCTCAAGCGATCCTCCCACTTCAGCCTCCCAACTAGCTGTAACTACAGG
CACTGGCCACCAAACCCAGATAATTTTTTTTTTCCTGTAGAGGTGGGGTT
TTGCCACGTTACCCAGGCTGGTCTTGAACTCTTAAGCTCAAGCGATCCTC
CTGCCTCGGCCCCCCAAAGTTCTGGGATTACAGGCATGAGCCACCATACC
TGGCGTACAGTATCCAGTGTAATGCAGTGATTAAAAATTCAGGATCCAGA
CCGGGATGGTGGCTTGTGCCTGTAGTCCCAGGGGTGGAGGTTGCAGTGAA
CGGAAATGGTGCCACTGCATTCCAGCCTGGGTGACAGAGTGAGACCCTGT
CTCAACAAAACCCCCCAAAAACCAAGAACAAAAAAGAATGCAGGATCTGA
TGCTAGATTGTCTGCATTAGAACTCTAGCCACTTAAGCTGGGTGTGGTGG
CTCATGCCTGTAATCCCAGCACTTTTGGAGGCCGCAGGCGGGTGGATCAC
CTGAGATTGGGAGTTCAAGACCAGCCTGACCAACATGGAGAAACCCTGTT
TCTACTAAAAATACAAAATTAGCCACCGGGCATGGTGGTGCATGCCTGTA
ATCCCAGCTACTTGGGAGGCTGAGGCAGGAGAATCACTTGAACCCGGGAG
GCAGAGGTTACGGTGAGATGAGATGGCACCATTGCACTCCAGCCTGGGCA
ACAAGAGCGAAACTCCATCTCAAAAAAAAAAAAAAAAAAAAAGGAATTCT
AGCCACTTAGAAGCTCTGTGATCTTGGGCAAATTGCTTATCTTTGCACCT
CAGCCTCCTCCTCTGTAATATAGGGTAATAGTATCTACCTTAAAGGGTTG
TTGTGAAAATTAAATAGTTTAGTACATGTAAAGTGCTTAGACAAAGTATT
TGGCATTAAGCGAGAGTTGGATATATTAGCCATCATTATTAACCACCTGG
GGGAACTTCAACTGATTTGGAGTCTAGGCATACAACTGGAAAGACCTGCC
TAGGAGTGTCTTGTGAATGCGATTTGCATAACGGTTTAGGCCCAGCTGAC
GTCAAGGGCTCCTTATAGCTCCAGGTCAGTTGTAGCCCTGGATGTAGTTC
CTGCCACGCAACAGTCCCACAATCTCCCCACCAACCCTTCTTCCTACCCA
ACTCCTGCAGCACCAGGAAGTGAAACAAAGAGGCAGAGCCCTGTGCCTCC
AACTCACCCTTGTCCCTCTCATCCCATCCCCCAGGCTCTACTTCCTCCTC
CTTTTCATCTTTCTTTCATCTCTTATCTTTTAGGGCTCCCAGAATGGGGA
CCAGAGATGGGAAGAACATAGGAGACGTTGTACACAAGTAAGGTGAACTC
CCTATCCTGCCCCCTCCCCTTTCCTTATTCCATTGGTGTCCACCTTATTA
GGGAGAGAGGCAAAACAGTTCTCACCCAAACTCAGATAATTCTCTGATGC
TGGAAATGTTTAATCTAAAGGGTAGATTTCCATTTTTTTTTTTTTTTTTT
TGAGACAGAGTCTTGCTCTGTCACCCAGGCTGGAGTGCAGTGGCGCCATC
TCGGCTCACTGCAACCTCTGCCTCCTGGCTTCAAGCGATTCTCTTGCCTC
AGCCTCCCGAGTAGCTGGGACTATAGGCGCCCACCACCGTGCTGGCTCAT
TTTTGTATTTTTAGTAGAGACAGGATTTCACCATGATGGCCAGGCTGGTC
TCGAACTCCTGACCTCATGATCCGCCTGCCTCGGCCTCCCAAAGTACTGG
GATTACAGGCGTGAGCCACTGTACCCGGCCCTTGGTAGATTTAACTTAGA
ATCGTAATATTTTTTTTTCTTCTCTTAGCTCATACCTACAGAATCATAAT
ATTTGAACCAGAAGTGTCATTGGGCAGTTTTGAATAGCTCTAAGGGAAGG
GAGACCTCCATTCAGGACAAGTTTCTCAGAAGAAAAGGGTCAACCTCTTG
GGGGAGGCTTTGGGAGCCAGCTGTGTGGTCACCGATGGCCTCATTCTGAC
GTCTTCGAAATTGTTCTGGGACCCTCCACTGGGGTCGGGGCAGTCCCGGC
TTTGGACCACCTTCCACTCCCACGCCCAACCTCACACTCTTAGCTGTTTC
ACTCGATGTTGCATCATGGAGGGTGATGAAATCGGTGTCAGTGGATTTTA
CCCATGGATGCAACAAGCTGAAGGACCAGCCAGAGTCATTGACAGTGCAC
CTTCGACTACCCAGAACTCCTGGGCTTCCTAGCCATGGGGTCCAAAGCTG
GGACTGCCCCGACCCCAGTGGAGGGTCCCAGAACAATTTGGATGACGTCA
GAATGAGGCCATGGGACTAGGTGCTGGAATGTCTAAGTTGAACTTCCAGG
CCTTATTTGCACTAGTCCTGAAAAAAACATCATCCAACTCTTATAGAGCC
TATGAAATCTTGGGCCACTAGGGTTGAGGAGTCAGGTGGTTCTTAGTCAA
TAACCCTCTTCCCACAAGAGCCTTTCTAACCTCCACTGTGAGGCCTGAAA
TGGGGAGCAATAAGACCTCATACTGGCTTCCCAGTTCTCCAAGTTCCTTC
ATGCGCATTCTCTCCCATGAAACCAGGACCATCCAGTTGAAATAATGTTG
TTTCCAACTGAGAAAAAGAAGCCCGTTTATTCCTAATAGGGGGCATCAGG
TAGGAATCAAACTTCATTGCAAACAGCTCACCATCCTATTGGGAGATGAA
TGGATGTTTCTCTGTTTTGCTTTTTCCTCAAGCAGGAGGAAGTGAGGAAA
TTAGGTTTGGGGTGGGGTAGGGGTATAGCTTTGAGAGGCAAAAAGATCAG
GGAAAGATCAAACAGGAAGGAACTTGAGACCAGATTAATTTAAATATTTG
TTCTCCCTTACCCCTCCCACCCCATCCCCGCTGTGCCCCCCATCCCCGCC
CCTTCTATAGCTATTTCGATTCCTGGAGAGCATTACACATGTGTCCCATC
CCAGGCCTCTAGCCACAGCAACCACACTACTCATTTCCCCTGGAACTGAG
GCTGCATACCTGGGCTCCCCACAGAGGGGGATGATGCAGGGAGGGGAATC
CCACCTGCTGTGAGTCACCTGCTGGTATAAAGGGCGGGCCTTACAATGCA
GGGACCTTAAAAGACTCAGAGACAAAGGGAGAAAAACAACAGGAAGCAGC
TTACAAACTCGGTGAACAACTGAGGGAACCAAACCAGAGACGCGCTGAAC
AGAGAGAATCAGGCTCAAAGCAAGTGGAAGTGGGCAGAGATTCCACCAGG
ACTGGTGCAAGGCGCAGAGCCAGCCAGATTTGAGAAGAAGGCAAAAAG
ATG

16) AKT. Akt (Protein kinase B, PKB) is a serine/threonine kinase is an important node several signaling cascades downstream of growth factor receptor tyrosine kinases. Akt plays an essential role in cell survival and altered activity has been associated with cancer and other disease conditions, such as diabetes mellitus, neurodegenerative diseases, and muscle hypotrophy. AKT plays a key role in regulating tumor formation, cell survival, insulin signaling and metabolism (lipid and glucose), growth, migration, proliferation, polarity, cell cycle progression, muscle and cardiomyocyte contractility, angiogenesis, and self-renewal of stem cells (reviewed by Liao and Hung, Am J Transl Res. 2010; 2(1): 19-42). Akt is a downstream mediator of the PI 3-K pathway, resulting in the recruitment of Akt to the plasma membrane via the PH (plexstrin homology domain) of Akt. Akt is fully activated by phosphorylation at two key sites: Ser308 (phosphorylated by PDK1) and Thr478 (phosphorylated by mTOR and DNA-PK). Akt can then phosphorylated a wide range of substrates including transcription factors (e.g. FOXO1), kinases (GSK-3, Raf-1, ASK, Chk1) and other proteins with important signaling roles (e.g. Bad, MDM2).

Protein: AKT1 Gene: AKT1 (Homo sapiens, chromosome 14, 105235686-105262080 [NCBI Reference Sequence: NC_{—}000014.8]; start site location: 105258980; strand: negative)

Gene Identification

| | |
|---|---|
| GeneID | 207 |
| HGNC | 391 |
| HPRD | 01261 |
| MIM | 164730 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 3593 | | GAGGCTCCCGCGACGCTCACGCG | 8 |
| 3646 | | TACCGGGCGTCTCAGGTTTTGCC | 843 |
| 3669 | | TCCGAGCCGCGCACGCCTCAGGC | 1562 |
| 3703 | | CACCAACGGACTCCGTCCGCCC | 2010 |
| 3770 | | CCGCCGGCTGCCTCGCTGGCCCAGCG | 2464 |
| 3927 | | TCTCGGGTCCCGGCCTCGCCCGGCGG | 2556 |
| | | AGC | |
| 4084 | | CATTCTGGCGGCGCCGCGGCTCGCG | 2730 |
| 4228 | | CACCGGGCCGCCGCGTCCGGGCGCG | 2838 |
| 4338 | AKT4 | CACATCCGCCTCCGCCGCCCGG | 3160 |

Targeted Shift Sequences

| | | Relative |
|---|---|---|
| | | upstream |
| | | location |
| Sequence | | to gene |
| ID No: | Sequence (5′-3′) | start site |
| 3593 | GAGGCTCCCGCGACGCTCACGCG | 8 |
| 3594 | AGGCTCCCGCGACGCTCACG | 9 |
| 3595 | GGCTCCCGCGACGCTCACGC | 10 |
| 3596 | GCTCCCGCGACGCTCACGCG | 11 |
| 3597 | CTCCCGCGACGCTCACGCGC | 12 |
| 3598 | TCCCGCGACGCTCACGCGCT | 13 |
| 3599 | CCCGCGACGCTCACGCGCTC | 14 |
| 3600 | CCGCGACGCTCACGCGCTCC | 15 |
| 3601 | CGCGACGCTCACGCGCTCCT | 16 |
| 3602 | GCGACGCTCACGCGCTCCTC | 17 |
| 3603 | CGACGCTCACGCGCTCCTCT | 18 |
| 3604 | GACGCTCACGCGCTCCTCTC | 19 |
| 3605 | ACGCTCACGCGCTCCTCTCA | 20 |
| 3606 | CGCTCACGCGCTCCTCTCAG | 21 |
| 3607 | GCTCACGCGCTCCTCTCAGG | 22 |
| 3608 | CTCACGCGCTCCTCTCAGGC | 23 |
| 3609 | TCACGCGCTCCTCTCAGGCT | 24 |
| 3610 | CACGCGCTCCTCTCAGGCTG | 25 |
| 3611 | ACGCGCTCCTCTCAGGCTGG | 26 |
| 3612 | CGCGCTCCTCTCAGGCTGGC | 27 |
| 3613 | GCGCTCCTCTCAGGCTGGCG | 28 |
| 3614 | CGCTCCTCTCAGGCTGGCGC | 29 |
| 3615 | GCTCCTCTCAGGCTGGCGCT | 30 |
| 3616 | CTCCTCTCAGGCTGGCGCTC | 31 |
| 3617 | TCCTCTCAGGCTGGCGCTCC | 32 |
| 3618 | CCTCTCAGGCTGGCGCTCCC | 33 |
| 3619 | CTCTCAGGCTGGCGCTCCCC | 34 |
| 3620 | TCTCAGGCTGGCGCTCCCCG | 35 |
| 3621 | CTCAGGCTGGCGCTCCCCGA | 36 |
| 3622 | TCAGGCTGGCGCTCCCCGAG | 37 |
| 3623 | CAGGCTGGCGCTCCCCGAGC | 38 |
| 3624 | AGGCTGGCGCTCCCCGAGCC | 39 |
| 3625 | GGCTGGCGCTCCCCGAGCCC | 40 |
| 3626 | GCTGGCGCTCCCCGAGCCCA | 41 |
| 3627 | CTGGCGCTCCCCGAGCCCAG | 42 |
| 3628 | TGGCGCTCCCCGAGCCCAGC | 43 |
| 3629 | GGCGCTCCCCGAGCCCAGCT | 44 |
| 3630 | GCGCTCCCCGAGCCCAGCTG | 45 |
| 3631 | CGCTCCCCGAGCCCAGCTGG | 46 |
| 3632 | GCTCCCCGAGCCCAGCTGGC | 47 |
| 3633 | CTCCCCGAGCCCAGCTGGCC | 48 |
| 3634 | TCCCCGAGCCCAGCTGGCCT | 49 |
| 3635 | CCCCGAGCCCAGCTGGCCTG | 50 |
| 3636 | CCCGAGCCCAGCTGGCCTGG | 51 |
| 3637 | CCGAGCCCAGCTGGCCTGGC | 52 |
| 3638 | CGAGCCCAGCTGGCCTGGCC | 53 |
| 3639 | CGAGGCTCCCGCGACGCTCA | 7 |
| 3640 | CCGAGGCTCCCGCGACGCTC | 6 |
| 3641 | CCCGAGGCTCCCGCGACGCT | 5 |
| 3642 | GCCCGAGGCTCCCGCGACGC | 4 |
| 3643 | TGCCCGAGGCTCCCGCGACG | 3 |
| 3644 | GTGCCCGAGGCTCCCGCGAC | 2 |
| 3645 | GGTGCCCGAGGCTCCCGCGA | 1 |
| 3646 | TACCGGGCGTCTCAGGTTTTGCC | 843 |
| 3647 | ACCGGGCGTCTCAGGTTTTG | 844 |
| 3648 | CCGGGCGTCTCAGGTTTTGC | 845 |
| 3649 | CGGGCGTCTCAGGTTTTGCC | 846 |
| 3650 | GGGCGTCTCAGGTTTTGCCA | 847 |
| 3651 | GGCGTCTCAGGTTTTGCCAG | 848 |
| 3652 | GCGTCTCAGGTTTTGCCAGG | 849 |
| 3653 | CGTCTCAGGTTTTGCCAGGC | 850 |
| 3654 | GTACCGGGCGTCTCAGGTTT | 842 |
| 3655 | TGTACCGGGCGTCTCAGGTT | 841 |
| 3656 | ATGTACCGGGCGTCTCAGGT | 840 |
| 3657 | CATGTACCGGGCGTCTCAGG | 839 |
| 3658 | ACATGTACCGGGCGTCTCAG | 838 |
| 3659 | AACATGTACCGGGCGTCTCA | 837 |
| 3660 | CAACATGTACCGGGCGTCTC | 836 |
| 3661 | CCAACATGTACCGGGCGTCT | 835 |
| 3662 | GCCAACATGTACCGGGCGTC | 834 |
| 3663 | GGCCAACATGTACCGGGCGT | 833 |
| 3664 | TGGCCAACATGTACCGGGCG | 832 |
| 3665 | TTGGCCAACATGTACCGGGC | 831 |
| 3666 | TTTGGCCAACATGTACCGGG | 830 |
| 3667 | ATTTGGCCAACATGTACCGG | 829 |
| 3668 | CATTTGGCCAACATGTACCG | 828 |
| 3669 | TCCGAGCCGCGCACGCCTCAGGC | 1562 |
| 3670 | CCGAGCCGCGCACGCCTCAG | 1563 |
| 3671 | CGAGCCGCGCACGCCTCAGG | 1564 |
| 3672 | GAGCCGCGCACGCCTCAGGC | 1565 |
| 3673 | AGCCGCGCACGCCTCAGGCA | 1566 |
| 3674 | GCCGCGCACGCCTCAGGCAC | 1567 |
| 3675 | CCGCGCACGCCTCAGGCACA | 1568 |
| 3676 | CGCGCACGCCTCAGGCACAG | 1569 |
| 3677 | GCGCACGCCTCAGGCACAGG | 1570 |
| 3678 | CGCACGCCTCAGGCACAGGG | 1571 |
| 3679 | GCACGCCTCAGGCACAGGGG | 1572 |
| 3680 | CACGCCTCAGGCACAGGGGG | 1573 |
| 3681 | ACGCCTCAGGCACAGGGGGC | 1574 |
| 3682 | CGCCTCAGGCACAGGGGGCT | 1575 |
| 3683 | CTCCGAGCCGCGCACGCCTC | 1561 |
| 3684 | GCTCCGAGCCGCGCACGCCT | 1560 |
| 3685 | GGCTCCGAGCCGCGCACGCC | 1559 |
| 3686 | GGGCTCCGAGCCGCGCACGC | 1558 |
| 3687 | AGGGCTCCGAGCCGCGCACG | 1557 |
| 3688 | CAGGGCTCCGAGCCGCGCAC | 1556 |
| 3689 | GCAGGGCTCCGAGCCGCGCA | 1555 |
| 3690 | GGCAGGGCTCCGAGCCGCGC | 1554 |
| 3691 | GGGCAGGGCTCCGAGCCGCG | 1553 |
| 3692 | AGGGCAGGGCTCCGAGCCGC | 1552 |
| 3693 | GAGGGCAGGGCTCCGAGCCG | 1551 |
| 3694 | CGAGGGCAGGGCTCCGAGCC | 1550 |
| 3695 | CCGAGGGCAGGGCTCCGAGC | 1549 |
| 3696 | TCCGAGGGCAGGGCTCCGAG | 1548 |
| 3697 | CTCCGAGGGCAGGGCTCCGA | 1547 |
| 3698 | ACTCCGAGGGCAGGGCTCCG | 1546 |
| 3699 | GACTCCGAGGGCAGGGCTCC | 1545 |
| 3700 | GGACTCCGAGGGCAGGGCTC | 1544 |
| 3701 | AGGACTCCGAGGGCAGGGCT | 1543 |
| 3702 | CAGGACTCCGAGGGCAGGGC | 1542 |
| 3703 | CACCAACGGACTCCGTCCGCCC | 2010 |
| 3704 | ACCAACGGACTCCGTCCGCC | 2011 |
| 3705 | CCAACGGACTCCGTCCGCCC | 2012 |
| 3706 | CAACGGACTCCGTCCGCCCT | 2013 |
| 3707 | AACGGACTCCGTCCGCCCTT | 2014 |
| 3708 | ACGGACTCCGTCCGCCCTTC | 2015 |
| 3709 | CGGACTCCGTCCGCCCTTCG | 2016 |
| 3710 | GGACTCCGTCCGCCCTTCGC | 2017 |
| 3711 | GACTCCGTCCGCCCTTCGCT | 2018 |
| 3712 | ACTCCGTCCGCCCTTCGCTC | 2019 |
| 3713 | CTCCGTCCGCCCTTCGCTCG | 2020 |
| 3714 | TCCGTCCGCCCTTCGCTCGG | 2021 |
| 3715 | CCGTCCGCCCTTCGCTCGGA | 2022 |
| 3716 | CGTCCGCCCTTCGCTCGGAT | 2023 |
| 3717 | GTCCGCCCTTCGCTCGGATG | 2024 |
| 3718 | TCCGCCCTTCGCTCGGATGA | 2025 |
| 3719 | CCGCCCTTCGCTCGGATGAG | 2026 |
| 3720 | CGCCCTTCGCTCGGATGAGG | 2027 |
| 3721 | GCCCTTCGCTCGGATGAGGG | 2028 |
| 3722 | CCCTTCGCTCGGATGAGGGA | 2029 |
| 3723 | CCTTCGCTCGGATGAGGGAC | 2030 |
| 3724 | CTTCGCTCGGATGAGGGACT | 2031 |
| 3725 | TTCGCTCGGATGAGGGACTC | 2032 |
| 3726 | TCGCTCGGATGAGGGACTCA | 2033 |
| 3727 | CGCTCGGATGAGGGACTCAA | 2034 |
| 3728 | GCTCGGATGAGGGACTCAAA | 2035 |
| 3729 | CTCGGATGAGGGACTCAAAG | 2036 |
| 3730 | TCGGATGAGGGACTCAAAGC | 2037 |
| 3731 | CCACCAACGGACTCCGTCCG | 2009 |
| 3732 | CCCACCAACGGACTCCGTCC | 2008 |
| 3733 | CCCCACCAACGGACTCCGTC | 2007 |
| 3734 | CCCCCACCAACGGACTCCGT | 2006 |
| 3735 | ACCCCCACCAACGGACTCCG | 2005 |
| 3736 | GACCCCCACCAACGGACTCC | 2004 |
| 3737 | GGACCCCCACCAACGGACTC | 2003 |
| 3738 | CGGACCCCCACCAACGGACT | 2002 |
| 3739 | CCGGACCCCCACCAACGGAC | 2001 |
| 3740 | ACCGGACCCCCACCAACGGA | 2000 |
| 3741 | AACCGGACCCCCACCAACGG | 1999 |
| 3742 | CAACCGGACCCCCACCAACG | 1998 |
| 3743 | GCAACCGGACCCCCACCAAC | 1997 |
| 3744 | GGCAACCGGACCCCCACCAA | 1996 |
| 3745 | AGGCAACCGGACCCCCACCA | 1995 |
| 3746 | GAGGCAACCGGACCCCCACC | 1994 |
| 3747 | AGAGGCAACCGGACCCCCAC | 1993 |
| 3748 | GAGAGGCAACCGGACCCCCA | 1992 |
| 3749 | GGAGAGGCAACCGGACCCCC | 1991 |
| 3750 | GGGAGAGGCAACCGGACCCC | 1990 |
| 3751 | CGGGAGAGGCAACCGGACCC | 1989 |
| 3752 | CCGGGAGAGGCAACCGGACC | 1988 |
| 3753 | CCCGGGAGAGGCAACCGGAC | 1987 |
| 3754 | TCCCGGGAGAGGCAACCGGA | 1986 |
| 3755 | CTCCCGGGAGAGGCAACCGG | 1985 |
| 3756 | GCTCCCGGGAGAGGCAACCG | 1984 |
| 3757 | AGCTCCCGGGAGAGGCAACC | 1983 |
| 3758 | CAGCTCCCGGGAGAGGCAAC | 1982 |
| 3759 | ACAGCTCCCGGGAGAGGCAA | 1981 |
| 3760 | CACAGCTCCCGGGAGAGGCA | 1980 |
| 3761 | ACACAGCTCCCGGGAGAGGC | 1979 |
| 3762 | TACACAGCTCCCGGGAGAGG | 1978 |
| 3763 | CTACACAGCTCCCGGGAGAG | 1977 |
| 3764 | TCTACACAGCTCCCGGGAGA | 1976 |
| 3765 | GTCTACACAGCTCCCGGGAG | 1975 |
| 3766 | AGTCTACACAGCTCCCGGGA | 1974 |
| 3767 | AAGTCTACACAGCTCCCGGG | 1973 |
| 3768 | GAAGTCTACACAGCTCCCGG | 1972 |
| 3769 | AGAAGTCTACACAGCTCCCG | 1971 |
| 3770 | CCGCCGGCTGCCTCGCTGGCCCAGCG | 2464 |
| 3771 | CGCCGGCTGCCTCGCTGGCC | 2465 |
| 3772 | GCCGGCTGCCTCGCTGGCCC | 2466 |
| 3773 | CCGGCTGCCTCGCTGGCCCA | 2467 |
| 3774 | CGGCTGCCTCGCTGGCCCAG | 2468 |
| 3775 | GGCTGCCTCGCTGGCCCAGC | 2469 |
| 3776 | GCTGCCTCGCTGGCCCAGCG | 2470 |
| 3777 | CTGCCTCGCTGGCCCAGCGC | 2471 |
| 3778 | TGCCTCGCTGGCCCAGCGCC | 2472 |
| 3779 | GCCTCGCTGGCCCAGCGCCC | 2473 |
| 3780 | CCTCGCTGGCCCAGCGCCCG | 2474 |
| 3781 | CTCGCTGGCCCAGCGCCCGG | 2475 |
| 3782 | TCGCTGGCCCAGCGCCCGGG | 2476 |
| 3783 | CGCTGGCCCAGCGCCCGGGG | 2477 |
| 3784 | GCTGGCCCAGCGCCCGGGGA | 2478 |
| 3785 | CTGGCCCAGCGCCCGGGGAG | 2479 |
| 3786 | TGGCCCAGCGCCCGGGGAGC | 2480 |
| 3787 | GGCCCAGCGCCCGGGGAGCC | 2481 |
| 3788 | GCCCAGCGCCCGGGGAGCCC | 2482 |
| 3789 | CCCAGCGCCCGGGGAGCCCC | 2483 |
| 3790 | CCAGCGCCCGGGGAGCCCCA | 2484 |
| 3791 | CAGCGCCCGGGGAGCCCCAC | 2485 |
| 3792 | AGCGCCCGGGGAGCCCCACG | 2486 |
| 3793 | GCGCCCGGGGAGCCCCACGG | 2487 |
| 3794 | CGCCCGGGGAGCCCCACGGC | 2488 |
| 3795 | GCCCGGGGAGCCCCACGGCC | 2489 |
| 3796 | CCCGGGGAGCCCCACGGCCC | 2490 |
| 3797 | CCGGGGAGCCCCACGGCCCG | 2491 |
| 3798 | CGGGGAGCCCCACGGCCCGC | 2492 |
| 3799 | GGGGAGCCCCACGGCCCGCA | 2493 |
| 3800 | GGGAGCCCCACGGCCCGCAG | 2494 |
| 3801 | GGAGCCCCACGGCCCGCAGG | 2495 |
| 3802 | GAGCCCCACGGCCCGCAGGG | 2496 |
| 3803 | AGCCCCACGGCCCGCAGGGG | 2497 |
| 3804 | GCCCCACGGCCCGCAGGGGC | 2498 |
| 3805 | CCCCACGGCCCGCAGGGGCA | 2499 |
| 3806 | CCCACGGCCCGCAGGGGCAC | 2500 |
| 3807 | CCACGGCCCGCAGGGGCACC | 2501 |
| 3808 | CACGGCCCGCAGGGGCACCC | 2502 |
| 3809 | ACGGCCCGCAGGGGCACCCC | 2503 |
| 3810 | CGGCCCGCAGGGGCACCCCG | 2504 |
| 3811 | GGCCCGCAGGGGCACCCCGA | 2505 |
| 3812 | GCCCGCAGGGGCACCCCGAG | 2506 |
| 3813 | CCCGCAGGGGCACCCCGAGC | 2507 |
| 3814 | CCGCAGGGGCACCCCGAGCC | 2508 |
| 3815 | CGCAGGGGCACCCCGAGCCC | 2509 |
| 3816 | GCAGGGGCACCCCGAGCCCC | 2510 |
| 3817 | CAGGGGCACCCCGAGCCCCA | 2511 |
| 3818 | AGGGGCACCCCGAGCCCCAG | 2512 |
| 3819 | GGGGCACCCCGAGCCCCAGC | 2513 |
| 3820 | GGGCACCCCGAGCCCCAGCT | 2514 |
| 3821 | GGCACCCCGAGCCCCAGCTC | 2515 |
| 3822 | GCACCCCGAGCCCCAGCTCC | 2516 |
| 3823 | CACCCCGAGCCCCAGCTCCA | 2517 |
| 3824 | ACCCCGAGCCCCAGCTCCAG | 2518 |
| 3825 | CCCCGAGCCCCAGCTCCAGG | 2519 |
| 3826 | CCCGAGCCCCAGCTCCAGGC | 2520 |
| 3827 | CCGAGCCCCAGCTCCAGGCC | 2521 |
| 3828 | CGAGCCCCAGCTCCAGGCCC | 2522 |
| 3829 | GAGCCCCAGCTCCAGGCCCG | 2523 |
| 3830 | AGCCCCAGCTCCAGGCCCGG | 2524 |
| 3831 | GCCCCAGCTCCAGGCCCGGC | 2525 |
| 3832 | CCCCAGCTCCAGGCCCGGCG | 2526 |
| 3833 | CCCAGCTCCAGGCCCGGCGG | 2527 |
| 3834 | CCAGCTCCAGGCCCGGCGGC | 2528 |
| 3835 | CAGCTCCAGGCCCGGCGGCG | 2529 |
| 3836 | AGCTCCAGGCCCGGCGGCGT | 2530 |
| 3837 | GCTCCAGGCCCGGCGGCGTC | 2531 |
| 3838 | CTCCAGGCCCGGCGGCGTCC | 2532 |
| 3839 | TCCAGGCCCGGCGGCGTCCC | 2533 |
| 3840 | CCAGGCCCGGCGGCGTCCCT | 2534 |
| 3841 | CAGGCCCGGCGGCGTCCCTT | 2535 |
| 3842 | AGGCCCGGCGGCGTCCCTTC | 2536 |
| 3843 | GGCCCGGCGGCGTCCCTTCT | 2537 |
| 3844 | GCCCGGCGGCGTCCCTTCTC | 2538 |
| 3845 | CCCGGCGGCGTCCCTTCTCT | 2539 |
| 3846 | CCGGCGGCGTCCCTTCTCTC | 2540 |
| 3847 | CGGCGGCGTCCCTTCTCTCG | 2541 |
| 3848 | GGCGGCGTCCCTTCTCTCGG | 2542 |
| 3849 | GCGGCGTCCCTTCTCTCGGG | 2543 |
| 3850 | CGGCGTCCCTTCTCTCGGGT | 2544 |
| 3851 | GGCGTCCCTTCTCTCGGGTC | 2545 |
| 3852 | GCGTCCCTTCTCTCGGGTCC | 2546 |
| 3853 | CGTCCCTTCTCTCGGGTCCC | 2547 |
| 3854 | GTCCCTTCTCTCGGGTCCCG | 2548 |
| 3855 | TCCCTTCTCTCGGGTCCCGG | 2549 |
| 3856 | CCCTTCTCTCGGGTCCCGGC | 2550 |
| 3857 | CCTTCTCTCGGGTCCCGGCC | 2551 |
| 3858 | CTTCTCTCGGGTCCCGGCCT | 2552 |
| 3859 | TTCTCTCGGGTCCCGGCCTC | 2553 |
| 3860 | TCTCTCGGGTCCCGGCCTCG | 2554 |
| 3861 | CTCTCGGGTCCCGGCCTCGC | 2555 |
| 3862 | TCTCGGGTCCCGGCCTCGCC | 2556 |
| 3863 | CTCGGGTCCCGGCCTCGCCC | 2557 |
| 3864 | TCGGGTCCCGGCCTCGCCCG | 2558 |
| 3865 | CGGGTCCCGGCCTCGCCCGG | 2559 |
| 3866 | GGGTCCCGGCCTCGCCCGGC | 2560 |
| 3867 | GGTCCCGGCCTCGCCCGGCG | 2561 |
| 3868 | GTCCCGGCCTCGCCCGGCGG | 2562 |
| 3869 | TCCCGGCCTCGCCCGGCGGA | 2563 |
| 3870 | CCCGGCCTCGCCCGGCGGAG | 2564 |
| 3871 | CCGGCCTCGCCCGGCGGAGC | 2565 |
| 3872 | CGGCCTCGCCCGGCGGAGCG | 2566 |
| 3873 | GGCCTCGCCCGGCGGAGCGG | 2567 |
| 3874 | GCCTCGCCCGGCGGAGCGGC | 2568 |
| 3875 | CCTCGCCCGGCGGAGCGGCC | 2569 |
| 3876 | CTCGCCCGGCGGAGCGGCCT | 2570 |
| 3877 | TCGCCCGGCGGAGCGGCCTC | 2571 |
| 3878 | CGCCCGGCGGAGCGGCCTCC | 2572 |
| 3879 | GCCCGGCGGAGCGGCCTCCC | 2573 |
| 3880 | CCCGGCGGAGCGGCCTCCCC | 2574 |
| 3881 | CCGGCGGAGCGGCCTCCCCA | 2575 |
| 3882 | CGGCGGAGCGGCCTCCCCAA | 2576 |
| 3883 | GGCGGAGCGGCCTCCCCAAG | 2577 |
| 3884 | GCGGAGCGGCCTCCCCAAGG | 2578 |
| 3885 | CGGAGCGGCCTCCCCAAGGT | 2579 |
| 3886 | GGAGCGGCCTCCCCAAGGTC | 2580 |
| 3887 | GAGCGGCCTCCCCAAGGTCA | 2581 |
| 3888 | AGCGGCCTCCCCAAGGTCAT | 2582 |
| 3889 | GCGGCCTCCCCAAGGTCATG | 2583 |
| 3890 | CGGCCTCCCCAAGGTCATGA | 2584 |
| 3891 | TCCGCCGGCTGCCTCGCTGG | 2463 |
| 3892 | CTCCGCCGGCTGCCTCGCTG | 2462 |
| 3893 | CCTCCGCCGGCTGCCTCGCT | 2461 |
| 3894 | ACCTCCGCCGGCTGCCTCGC | 2460 |
| 3895 | CACCTCCGCCGGCTGCCTCG | 2459 |
| 3896 | GCACCTCCGCCGGCTGCCTC | 2458 |
| 3897 | GGCACCTCCGCCGGCTGCCT | 2457 |
| 3898 | GGGCACCTCCGCCGGCTGCC | 2456 |
| 3899 | GGGGCACCTCCGCCGGCTGC | 2455 |
| 3900 | CGGGGCACCTCCGCCGGCTG | 2454 |
| 3901 | CCGGGGCACCTCCGCCGGCT | 2453 |
| 3902 | CCCGGGGCACCTCCGCCGGC | 2452 |
| 3903 | CCCCGGGGCACCTCCGCCGG | 2451 |
| 3904 | ACCCCGGGGCACCTCCGCCG | 2450 |
| 3905 | AACCCCGGGGCACCTCCGCC | 2449 |
| 3906 | CAACCCCGGGGCACCTCCGC | 2448 |
| 3907 | CCAACCCCGGGGCACCTCCG | 2447 |
| 3908 | TCCAACCCCGGGGCACCTCC | 2446 |
| 3909 | CTCCAACCCCGGGGCACCTC | 2445 |
| 3910 | TCTCCAACCCCGGGGCACCT | 2444 |
| 3911 | TTCTCCAACCCCGGGGCACC | 2443 |
| 3912 | TTTCTCCAACCCCGGGGCAC | 2442 |
| 3913 | CTTTCTCCAACCCCGGGGCA | 2441 |
| 3914 | TCTTTCTCCAACCCCGGGGC | 2440 |
| 3915 | GTCTTTCTCCAACCCCGGGG | 2439 |
| 3916 | AGTCTTTCTCCAACCCCGGG | 2438 |
| 3917 | GAGTCTTTCTCCAACCCCGG | 2437 |
| 3918 | CGAGTCTTTCTCCAACCCCG | 2436 |
| 3919 | GCGAGTCTTTCTCCAACCCC | 2435 |
| 3920 | GGCGAGTCTTTCTCCAACCC | 2434 |
| 3921 | CGGCGAGTCTTTCTCCAACC | 2433 |
| 3922 | GCGGCGAGTCTTTCTCCAAC | 2432 |
| 3923 | CGCGGCGAGTCTTTCTCCAA | 2431 |
| 3924 | CCGCGGCGAGTCTTTCTCCA | 2430 |
| 3925 | GCCGCGGCGAGTCTTTCTCC | 2429 |
| 3926 | GGCCGCGGCGAGTCTTTCTC | 2428 |
| 3927 | TCTCGGGTCCCGGCCTCGCCCGGCGGAGC | 2556 |
| 3928 | CTCGGGTCCCGGCCTCGCCC | 2557 |
| 3929 | TCGGGTCCCGGCCTCGCCCG | 2558 |
| 3930 | CGGGTCCCGGCCTCGCCCGG | 2559 |
| 3931 | GGGTCCCGGCCTCGCCCGGC | 2560 |
| 3932 | GGTCCCGGCCTCGCCCGGCG | 2561 |
| 3933 | GTCCCGGCCTCGCCCGGCGG | 2562 |
| 3934 | TCCCGGCCTCGCCCGGCGGA | 2563 |
| 3935 | CCCGGCCTCGCCCGGCGGAG | 2564 |
| 3936 | CCGGCCTCGCCCGGCGGAGC | 2565 |
| 3937 | CGGCCTCGCCCGGCGGAGCG | 2566 |
| 3938 | GGCCTCGCCCGGCGGAGCGG | 2567 |
| 3939 | GCCTCGCCCGGCGGAGCGGC | 2568 |
| 3940 | CCTCGCCCGGCGGAGCGGCC | 2569 |
| 3941 | CTCGCCCGGCGGAGCGGCCT | 2570 |
| 3942 | TCGCCCGGCGGAGCGGCCTC | 2571 |
| 3943 | CGCCCGGCGGAGCGGCCTCC | 2572 |
| 3944 | GCCCGGCGGAGCGGCCTCCC | 2573 |
| 3945 | CCCGGCGGAGCGGCCTCCCC | 2574 |
| 3946 | CCGGCGGAGCGGCCTCCCCA | 2575 |
| 3947 | CGGCGGAGCGGCCTCCCCAA | 2576 |
| 3948 | GGCGGAGCGGCCTCCCCAAG | 2577 |
| 3949 | GCGGAGCGGCCTCCCCAAGG | 2578 |
| 3950 | CGGAGCGGCCTCCCCAAGGT | 2579 |
| 3951 | GGAGCGGCCTCCCCAAGGTC | 2580 |
| 3952 | GAGCGGCCTCCCCAAGGTCA | 2581 |
| 3953 | AGCGGCCTCCCCAAGGTCAT | 2582 |
| 3954 | GCGGCCTCCCCAAGGTCATG | 2583 |
| 3955 | CGGCCTCCCCAAGGTCATGA | 2584 |
| 3956 | CTCTCGGGTCCCGGCCTCGC | 2555 |
| 3957 | TCTCTCGGGTCCCGGCCTCG | 2554 |
| 3958 | TTCTCTCGGGTCCCGGCCTC | 2553 |
| 3959 | CTTCTCTCGGGTCCCGGCCT | 2552 |
| 3960 | CCTTCTCTCGGGTCCCGGCC | 2551 |
| 3961 | CCCTTCTCTCGGGTCCCGGC | 2550 |
| 3962 | TCCCTTCTCTCGGGTCCCGG | 2549 |
| 3963 | GTCCCTTCTCTCGGGTCCCG | 2548 |
| 3964 | CGTCCCTTCTCTCGGGTCCC | 2547 |
| 3965 | GCGTCCCTTCTCTCGGGTCC | 2546 |
| 3966 | GGCGTCCCTTCTCTCGGGTC | 2545 |
| 3967 | CGGCGTCCCTTCTCTCGGGT | 2544 |
| 3968 | GCGGCGTCCCTTCTCTCGGG | 2543 |
| 3969 | GGCGGCGTCCCTTCTCTCGG | 2542 |
| 3970 | CGGCGGCGTCCCTTCTCTCG | 2541 |
| 3971 | CCGGCGGCGTCCCTTCTCTC | 2540 |
| 3972 | CCCGGCGGCGTCCCTTCTCT | 2539 |
| 3973 | GCCCGGCGGCGTCCCTTCTC | 2538 |
| 3974 | GGCCCGGCGGCGTCCCTTCT | 2537 |
| 3975 | AGGCCCGGCGGCGTCCCTTC | 2536 |
| 3976 | CAGGCCCGGCGGCGTCCCTT | 2535 |
| 3977 | CCAGGCCCGGCGGCGTCCCT | 2534 |
| 3978 | TCCAGGCCCGGCGGCGTCCC | 2533 |
| 3979 | CTCCAGGCCCGGCGGCGTCC | 2532 |
| 3980 | GCTCCAGGCCCGGCGGCGTC | 2531 |
| 3981 | AGCTCCAGGCCCGGCGGCGT | 2530 |
| 3982 | CAGCTCCAGGCCCGGCGGCG | 2529 |
| 3983 | CCAGCTCCAGGCCCGGCGGC | 2528 |
| 3984 | CCCAGCTCCAGGCCCGGCGG | 2527 |
| 3985 | CCCCAGCTCCAGGCCCGGCG | 2526 |
| 3986 | GCCCCAGCTCCAGGCCCGGC | 2525 |
| 3987 | AGCCCCAGCTCCAGGCCCGG | 2524 |
| 3988 | GAGCCCCAGCTCCAGGCCCG | 2523 |
| 3989 | CGAGCCCCAGCTCCAGGCCC | 2522 |
| 3990 | CCGAGCCCCAGCTCCAGGCC | 2521 |
| 3991 | CCCGAGCCCCAGCTCCAGGC | 2520 |
| 3992 | CCCCGAGCCCCAGCTCCAGG | 2519 |
| 3993 | ACCCCGAGCCCCAGCTCCAG | 2518 |
| 3994 | CACCCCGAGCCCCAGCTCCA | 2517 |
| 3995 | GCACCCCGAGCCCCAGCTCC | 2516 |
| 3996 | GGCACCCCGAGCCCCAGCTC | 2515 |
| 3997 | GGGCACCCCGAGCCCCAGCT | 2514 |
| 3998 | GGGGCACCCCGAGCCCCAGC | 2513 |
| 3999 | AGGGGCACCCCGAGCCCCAG | 2512 |
| 4000 | CAGGGGCACCCCGAGCCCCA | 2511 |
| 4001 | GCAGGGGCACCCCGAGCCCC | 2510 |
| 4002 | CGCAGGGGCACCCCGAGCCC | 2509 |
| 4003 | CCGCAGGGGCACCCCGAGCC | 2508 |
| 4004 | CCCGCAGGGGCACCCCGAGC | 2507 |
| 4005 | GCCCGCAGGGGCACCCCGAG | 2506 |
| 4006 | GGCCCGCAGGGGCACCCCGA | 2505 |
| 4007 | CGGCCCGCAGGGGCACCCCG | 2504 |
| 4008 | ACGGCCCGCAGGGGCACCCC | 2503 |
| 4009 | CACGGCCCGCAGGGGCACCC | 2502 |
| 4010 | CCACGGCCCGCAGGGGCACC | 2501 |
| 4011 | CCCACGGCCCGCAGGGGCAC | 2500 |
| 4012 | CCCCACGGCCCGCAGGGGCA | 2499 |
| 4013 | GCCCCACGGCCCGCAGGGGC | 2498 |
| 4014 | AGCCCCACGGCCCGCAGGGG | 2497 |
| 4015 | GAGCCCCACGGCCCGCAGGG | 2496 |
| 4016 | GGAGCCCCACGGCCCGCAGG | 2495 |
| 4017 | GGGAGCCCCACGGCCCGCAG | 2494 |
| 4018 | GGGGAGCCCCACGGCCCGCA | 2493 |
| 4019 | CGGGGAGCCCCACGGCCCGC | 2492 |
| 4020 | CCGGGGAGCCCCACGGCCCG | 2491 |
| 4021 | CCCGGGGAGCCCCACGGCCC | 2490 |
| 4022 | GCCCGGGGAGCCCCACGGCC | 2489 |
| 4023 | CGCCCGGGGAGCCCCACGGC | 2488 |
| 4024 | GCGCCCGGGGAGCCCCACGG | 2487 |
| 4025 | AGCGCCCGGGGAGCCCCACG | 2486 |
| 4026 | CAGCGCCCGGGGAGCCCCAC | 2485 |
| 4027 | CCAGCGCCCGGGGAGCCCCA | 2484 |
| 4028 | CCCAGCGCCCGGGGAGCCCC | 2483 |
| 4029 | GCCCAGCGCCCGGGGAGCCC | 2482 |
| 4030 | GGCCCAGCGCCCGGGGAGCC | 2481 |
| 4031 | TGGCCCAGCGCCCGGGGAGC | 2480 |
| 4032 | CTGGCCCAGCGCCCGGGGAG | 2479 |
| 4033 | GCTGGCCCAGCGCCCGGGGA | 2478 |
| 4034 | CGCTGGCCCAGCGCCCGGGG | 2477 |
| 4035 | TCGCTGGCCCAGCGCCCGGG | 2476 |
| 4036 | CTCGCTGGCCCAGCGCCCGG | 2475 |
| 4037 | CCTCGCTGGCCCAGCGCCCG | 2474 |
| 4038 | GCCTCGCTGGCCCAGCGCCC | 2473 |
| 4039 | TGCCTCGCTGGCCCAGCGCC | 2472 |
| 4040 | CTGCCTCGCTGGCCCAGCGC | 2471 |
| 4041 | GCTGCCTCGCTGGCCCAGCG | 2470 |
| 4042 | GGCTGCCTCGCTGGCCCAGC | 2469 |
| 4043 | CGGCTGCCTCGCTGGCCCAG | 2468 |
| 4044 | CCGGCTGCCTCGCTGGCCCA | 2467 |
| 4045 | GCCGGCTGCCTCGCTGGCCC | 2466 |
| 4046 | CGCCGGCTGCCTCGCTGGCC | 2465 |
| 4047 | CCGCCGGCTGCCTCGCTGGC | 2464 |
| 4048 | TCCGCCGGCTGCCTCGCTGG | 2463 |
| 4049 | CTCCGCCGGCTGCCTCGCTG | 2462 |
| 4050 | CCTCCGCCGGCTGCCTCGCT | 2461 |
| 4051 | ACCTCCGCCGGCTGCCTCGC | 2460 |
| 4052 | CACCTCCGCCGGCTGCCTCG | 2459 |
| 4053 | GCACCTCCGCCGGCTGCCTC | 2458 |
| 4054 | GGCACCTCCGCCGGCTGCCT | 2457 |
| 4055 | GGGCACCTCCGCCGGCTGCC | 2456 |
| 4056 | GGGGCACCTCCGCCGGCTGC | 2455 |
| 4057 | CGGGGCACCTCCGCCGGCTG | 2454 |
| 4058 | CCGGGGCACCTCCGCCGGCT | 2453 |
| 4059 | CCCGGGGCACCTCCGCCGGC | 2452 |
| 4060 | CCCCGGGGCACCTCCGCCGG | 2451 |
| 4061 | ACCCCGGGGCACCTCCGCCG | 2450 |
| 4062 | AACCCCGGGGCACCTCCGCC | 2449 |
| 4063 | CAACCCCGGGGCACCTCCGC | 2448 |
| 4064 | CCAACCCCGGGGCACCTCCG | 2447 |
| 4065 | TCCAACCCCGGGGCACCTCC | 2446 |
| 4066 | CTCCAACCCCGGGGCACCTC | 2445 |
| 4067 | TCTCCAACCCCGGGGCACCT | 2444 |
| 4068 | TTCTCCAACCCCGGGGCACC | 2443 |
| 4069 | TTTCTCCAACCCCGGGGCAC | 2442 |
| 4070 | CTTTCTCCAACCCCGGGGCA | 2441 |
| 4071 | TCTTTCTCCAACCCCGGGGC | 2440 |
| 4072 | GTCTTTCTCCAACCCCGGGG | 2439 |
| 4073 | AGTCTTTCTCCAACCCCGGG | 2438 |
| 4074 | GAGTCTTTCTCCAACCCCGG | 2437 |
| 4075 | CGAGTCTTTCTCCAACCCCG | 2436 |
| 4076 | GCGAGTCTTTCTCCAACCCC | 2435 |
| 4077 | GGCGAGTCTTTCTCCAACCC | 2434 |
| 4078 | CGGCGAGTCTTTCTCCAACC | 2433 |
| 4079 | GCGGCGAGTCTTTCTCCAAC | 2432 |
| 4080 | CGCGGCGAGTCTTTCTCCAA | 2431 |
| 4081 | CCGCGGCGAGTCTTTCTCCA | 2430 |
| 4082 | GCCGCGGCGAGTCTTTCTCC | 2429 |
| 4083 | GGCCGCGGCGAGTCTTTCTC | 2428 |
| 4084 | CATTCTGGCGGCGCCGCGGCTCGCG | 2730 |
| 4085 | ATTCTGGCGGCGCCGCGGCT | 2731 |
| 4086 | TTCTGGCGGCGCCGCGGCTC | 2732 |
| 4087 | TCTGGCGGCGCCGCGGCTCG | 2733 |
| 4088 | CTGGCGGCGCCGCGGCTCGC | 2734 |
| 4089 | TGGCGGCGCCGCGGCTCGCG | 2735 |
| 4090 | GGCGGCGCCGCGGCTCGCGC | 2736 |
| 4091 | GCGGCGCCGCGGCTCGCGCC | 2737 |
| 4092 | CGGCGCCGCGGCTCGCGCCC | 2738 |
| 4093 | GGCGCCGCGGCTCGCGCCCC | 2739 |
| 4094 | GCGCCGCGGCTCGCGCCCCG | 2740 |
| 4095 | CGCCGCGGCTCGCGCCCCGG | 2741 |
| 4096 | GCCGCGGCTCGCGCCCCGGC | 2742 |
| 4097 | CCGCGGCTCGCGCCCCGGCC | 2743 |
| 4098 | CGCGGCTCGCGCCCCGGCCC | 2744 |
| 4099 | GCGGCTCGCGCCCCGGCCCG | 2745 |
| 4100 | CGGCTCGCGCCCCGGCCCGA | 2746 |
| 4101 | GGCTCGCGCCCCGGCCCGAC | 2747 |
| 4102 | GCTCGCGCCCCGGCCCGACC | 2748 |
| 4103 | CCATTCTGGCGGCGCCGCGG | 2729 |
| 4104 | TCCATTCTGGCGGCGCCGCG | 2728 |
| 4105 | CTCCATTCTGGCGGCGCCGC | 2727 |
| 4106 | CCTCCATTCTGGCGGCGCCG | 2726 |
| 4107 | TCCTCCATTCTGGCGGCGCC | 2725 |
| 4108 | CTCCTCCATTCTGGCGGCGC | 2724 |
| 4109 | GCTCCTCCATTCTGGCGGCG | 2723 |
| 4110 | CGCTCCTCCATTCTGGCGGC | 2722 |
| 4111 | CCGCTCCTCCATTCTGGCGG | 2721 |
| 4112 | CCCGCTCCTCCATTCTGGCG | 2720 |
| 4113 | TCCCGCTCCTCCATTCTGGC | 2719 |
| 4114 | CTCCCGCTCCTCCATTCTGG | 2718 |
| 4115 | GCTCCCGCTCCTCCATTCTG | 2717 |
| 4116 | TGCTCCCGCTCCTCCATTCT | 2716 |
| 4117 | CTGCTCCCGCTCCTCCATTC | 2715 |
| 4118 | CCTGCTCCCGCTCCTCCATT | 2714 |
| 4119 | TCCTGCTCCCGCTCCTCCAT | 2713 |
| 4120 | TTCCTGCTCCCGCTCCTCCA | 2712 |
| 4121 | CTTCCTGCTCCCGCTCCTCC | 2711 |
| 4122 | ACTTCCTGCTCCCGCTCCTC | 2710 |
| 4123 | CACTTCCTGCTCCCGCTCCT | 2709 |
| 4124 | CCACTTCCTGCTCCCGCTCC | 2708 |
| 4125 | GCCACTTCCTGCTCCCGCTC | 2707 |
| 4126 | GGCCACTTCCTGCTCCCGCT | 2706 |
| 4127 | CGGCCACTTCCTGCTCCCGC | 2705 |
| 4128 | TCGGCCACTTCCTGCTCCCG | 2704 |
| 4129 | CTCGGCCACTTCCTGCTCCC | 2703 |
| 4130 | GCTCGGCCACTTCCTGCTCC | 2702 |
| 4131 | CGCTCGGCCACTTCCTGCTC | 2701 |
| 4132 | CCGCTCGGCCACTTCCTGCT | 2700 |
| 4133 | CCCGCTCGGCCACTTCCTGC | 2699 |
| 4134 | GCCCGCTCGGCCACTTCCTG | 2698 |
| 4135 | GGCCCGCTCGGCCACTTCCT | 2697 |
| 4136 | AGGCCCGCTCGGCCACTTCC | 2696 |
| 4137 | CAGGCCCGCTCGGCCACTTC | 2695 |
| 4138 | CCAGGCCCGCTCGGCCACTT | 2694 |
| 4139 | CCCAGGCCCGCTCGGCCACT | 2693 |
| 4140 | GCCCAGGCCCGCTCGGCCAC | 2692 |
| 4141 | CGCCCAGGCCCGCTCGGCCA | 2691 |
| 4142 | CCGCCCAGGCCCGCTCGGCC | 2690 |
| 4143 | CCCGCCCAGGCCCGCTCGGC | 2689 |
| 4144 | CCCCGCCCAGGCCCGCTCGG | 2688 |
| 4145 | TCCCCGCCCAGGCCCGCTCG | 2687 |
| 4146 | CTCCCCGCCCAGGCCCGCTC | 2686 |
| 4147 | CCTCCCCGCCCAGGCCCGCT | 2685 |
| 4148 | CCCTCCCCGCCCAGGCCCGC | 2684 |
| 4149 | GCCCTCCCCGCCCAGGCCCG | 2683 |
| 4150 | CGCCCTCCCCGCCCAGGCCC | 2682 |
| 4151 | GCGCCCTCCCCGCCCAGGCC | 2681 |
| 4152 | CGCGCCCTCCCCGCCCAGGC | 2680 |
| 4153 | CCGCGCCCTCCCCGCCCAGG | 2679 |
| 4154 | CCCGCGCCCTCCCCGCCCAG | 2678 |
| 4155 | CCCCGCGCCCTCCCCGCCCA | 2677 |
| 4156 | GCCCCGCGCCCTCCCCGCCC | 2676 |
| 4157 | CGCCCCGCGCCCTCCCCGCC | 2675 |
| 4158 | GCGCCCCGCGCCCTCCCCGC | 2674 |
| 4159 | CGCGCCCCGCGCCCTCCCCG | 2673 |
| 4160 | GCGCGCCCCGCGCCCTCCCC | 2672 |
| 4161 | CGCGCGCCCCGCGCCCTCCC | 2671 |
| 4162 | CCGCGCGCCCCGCGCCCTCC | 2670 |
| 4163 | CCCGCGCGCCCCGCGCCCTC | 2669 |
| 4164 | GCCCGCGCGCCCCGCGCCCT | 2668 |
| 4165 | GGCCCGCGCGCCCCGCGCCC | 2667 |
| 4166 | GGGCCCGCGCGCCCCGCGCC | 2666 |
| 4167 | CGGGCCCGCGCGCCCCGCGC | 2665 |
| 4168 | CCGGGCCCGCGCGCCCCGCG | 2664 |
| 4169 | GCCGGGCCCGCGCGCCCCGC | 2663 |
| 4170 | GGCCGGGCCCGCGCGCCCCG | 2662 |
| 4171 | TGGCCGGGCCCGCGCGCCCC | 2661 |
| 4172 | TTGGCCGGGCCCGCGCGCCC | 2660 |
| 4173 | CTTGGCCGGGCCCGCGCGCC | 2659 |
| 4174 | CCTTGGCCGGGCCCGCGCGC | 2658 |
| 4175 | CCCTTGGCCGGGCCCGCGCG | 2657 |
| 4176 | TCCCTTGGCCGGGCCCGCGC | 2656 |
| 4177 | CTCCCTTGGCCGGGCCCGCG | 2655 |
| 4178 | CCTCCCTTGGCCGGGCCCGC | 2654 |
| 4179 | CCCTCCCTTGGCCGGGCCCG | 2653 |
| 4180 | GCCCTCCCTTGGCCGGGCCC | 2652 |
| 4181 | CGCCCTCCCTTGGCCGGGCC | 2651 |
| 4182 | CCGCCCTCCCTTGGCCGGGC | 2650 |
| 4183 | GCCGCCCTCCCTTGGCCGGG | 2649 |
| 4184 | GGCCGCCCTCCCTTGGCCGG | 2648 |
| 4185 | GGGCCGCCCTCCCTTGGCCG | 2647 |
| 4186 | GGGGCCGCCCTCCCTTGGCC | 2646 |
| 4187 | TGGGGCCGCCCTCCCTTGGC | 2645 |
| 4188 | GTGGGGCCGCCCTCCCTTGG | 2644 |
| 4189 | CGTGGGGCCGCCCTCCCTTG | 2643 |
| 4190 | GCGTGGGGCCGCCCTCCCTT | 2642 |
| 4191 | GGCGTGGGGCCGCCCTCCCT | 2641 |
| 4192 | CGGCGTGGGGCCGCCCTCCC | 2640 |
| 4193 | CCGGCGTGGGGCCGCCCTCC | 2639 |
| 4194 | CCCGGCGTGGGGCCGCCCTC | 2638 |
| 4195 | GCCCGGCGTGGGGCCGCCCT | 2637 |
| 4196 | CGCCCGGCGTGGGGCCGCCC | 2636 |
| 4197 | GCGCCCGGCGTGGGGCCGCC | 2635 |
| 4198 | GGCGCCCGGCGTGGGGCCGC | 2634 |
| 4199 | CGGCGCCCGGCGTGGGGCCG | 2633 |
| 4200 | CCGGCGCCCGGCGTGGGGCC | 2632 |
| 4201 | CCCGGCGCCCGGCGTGGGGC | 2631 |
| 4202 | CCCCGGCGCCCGGCGTGGGG | 2630 |
| 4203 | CCCCCGGCGCCCGGCGTGGG | 2629 |
| 4204 | ACCCCCGGCGCCCGGCGTGG | 2628 |
| 4205 | CACCCCCGGCGCCCGGCGTG | 2627 |
| 4206 | GCACCCCCGGCGCCCGGCGT | 2626 |
| 4207 | TGCACCCCCGGCGCCCGGCG | 2625 |
| 4208 | CTGCACCCCCGGCGCCCGGC | 2624 |
| 4209 | CCTGCACCCCCGGCGCCCGG | 2623 |
| 4210 | GCCTGCACCCCCGGCGCCCG | 2622 |
| 4211 | AGCCTGCACCCCCGGCGCCC | 2621 |
| 4212 | CAGCCTGCACCCCCGGCGCC | 2620 |
| 4213 | GCAGCCTGCACCCCCGGCGC | 2619 |
| 4214 | GGCAGCCTGCACCCCCGGCG | 2618 |
| 4215 | CGGCAGCCTGCACCCCCGGC | 2617 |
| 4216 | CCGGCAGCCTGCACCCCCGG | 2616 |
| 4217 | GCCGGCAGCCTGCACCCCCG | 2615 |
| 4218 | GGCCGGCAGCCTGCACCCCC | 2614 |
| 4219 | GGGCCGGCAGCCTGCACCCC | 2613 |
| 4220 | GGGGCCGGCAGCCTGCACCC | 2612 |
| 4221 | TGGGGCCGGCAGCCTGCACC | 2611 |
| 4222 | CTGGGGCCGGCAGCCTGCAC | 2610 |
| 4223 | GCTGGGGCCGGCAGCCTGCA | 2609 |
| 4224 | GGCTGGGGCCGGCAGCCTGC | 2608 |
| 4225 | AGGCTGGGGCCGGCAGCCTG | 2607 |
| 4226 | GAGGCTGGGGCCGGCAGCCT | 2606 |
| 4227 | GGAGGCTGGGGCCGGCAGCC | 2605 |
| 4228 | CACCGGGCCGCCGCGTCCGGGCGCG | 2838 |
| 4229 | ACCGGGCCGCCGCGTCCGGG | 2839 |
| 4230 | CCGGGCCGCCGCGTCCGGGC | 2840 |
| 4231 | CGGGCCGCCGCGTCCGGGCG | 2841 |
| 4232 | GGGCCGCCGCGTCCGGGCGC | 2842 |
| 4233 | GGCCGCCGCGTCCGGGCGCG | 2843 |
| 4234 | GCCGCCGCGTCCGGGCGCGA | 2844 |
| 4235 | CCGCCGCGTCCGGGCGCGAG | 2845 |
| 4236 | CGCCGCGTCCGGGCGCGAGC | 2846 |
| 4237 | GCCGCGTCCGGGCGCGAGCG | 2847 |
| 4238 | CCGCGTCCGGGCGCGAGCGC | 2848 |
| 4239 | CGCGTCCGGGCGCGAGCGCG | 2849 |
| 4240 | GCGTCCGGGCGCGAGCGCGG | 2850 |
| 4241 | CGTCCGGGCGCGAGCGCGGG | 2851 |
| 4242 | GTCCGGGCGCGAGCGCGGGC | 2852 |
| 4243 | TCCGGGCGCGAGCGCGGGCC | 2853 |
| 4244 | CCGGGCGCGAGCGCGGGCCT | 2854 |
| 4245 | CGGGCGCGAGCGCGGGCCTA | 2855 |
| 4246 | GGGCGCGAGCGCGGGCCTAG | 2856 |
| 4247 | GGCGCGAGCGCGGGCCTAGC | 2857 |
| 4248 | GCGCGAGCGCGGGCCTAGCC | 2858 |
| 4249 | CGCGAGCGCGGGCCTAGCCG | 2859 |
| 4250 | GCGAGCGCGGGCCTAGCCGG | 2860 |
| 4251 | CGAGCGCGGGCCTAGCCGGG | 2861 |
| 4252 | GAGCGCGGGCCTAGCCGGGC | 2862 |
| 4253 | AGCGCGGGCCTAGCCGGGCC | 2863 |
| 4254 | GCGCGGGCCTAGCCGGGCCG | 2864 |
| 4255 | CGCGGGCCTAGCCGGGCCGC | 2865 |
| 4256 | GCGGGCCTAGCCGGGCCGCG | 2866 |
| 4257 | CGGGCCTAGCCGGGCCGCGG | 2867 |
| 4258 | GGGCCTAGCCGGGCCGCGGC | 2868 |
| 4259 | GGCCTAGCCGGGCCGCGGCC | 2869 |
| 4260 | GCCTAGCCGGGCCGCGGCCT | 2870 |
| 4261 | CCTAGCCGGGCCGCGGCCTC | 2871 |
| 4262 | CTAGCCGGGCCGCGGCCTCC | 2872 |
| 4263 | TAGCCGGGCCGCGGCCTCCG | 2873 |
| 4264 | AGCCGGGCCGCGGCCTCCGG | 2874 |
| 4265 | GCCGGGCCGCGGCCTCCGGC | 2875 |
| 4266 | CCGGGCCGCGGCCTCCGGCG | 2876 |
| 4267 | CGGGCCGCGGCCTCCGGCGC | 2877 |
| 4268 | GGGCCGCGGCCTCCGGCGCC | 2878 |
| 4269 | GGCCGCGGCCTCCGGCGCCC | 2879 |
| 4270 | GCCGCGGCCTCCGGCGCCCG | 2880 |
| 4271 | CCGCGGCCTCCGGCGCCCGC | 2881 |
| 4272 | CGCGGCCTCCGGCGCCCGCC | 2882 |
| 4273 | GCGGCCTCCGGCGCCCGCCG | 2883 |
| 4274 | CGGCCTCCGGCGCCCGCCGC | 2884 |
| 4275 | GGCCTCCGGCGCCCGCCGCT | 2885 |
| 4276 | GCCTCCGGCGCCCGCCGCTC | 2886 |
| 4277 | CCTCCGGCGCCCGCCGCTCC | 2887 |
| 4278 | CTCCGGCGCCCGCCGCTCCG | 2888 |
| 4279 | TCCGGCGCCCGCCGCTCCGC | 2889 |
| 4280 | CCGGCGCCCGCCGCTCCGCA | 2890 |
| 4281 | CGGCGCCCGCCGCTCCGCAT | 2891 |
| 4282 | GGCGCCCGCCGCTCCGCATC | 2892 |
| 4283 | GCGCCCGCCGCTCCGCATCC | 2893 |
| 4284 | CGCCCGCCGCTCCGCATCCC | 2894 |
| 4285 | GCCCGCCGCTCCGCATCCCC | 2895 |
| 4286 | CCCGCCGCTCCGCATCCCCG | 2896 |
| 4287 | CCGCCGCTCCGCATCCCCGC | 2897 |
| 4288 | CGCCGCTCCGCATCCCCGCG | 2898 |
| 4289 | GCCGCTCCGCATCCCCGCGG | 2899 |
| 4290 | CCGCTCCGCATCCCCGCGGG | 2900 |
| 4291 | CGCTCCGCATCCCCGCGGGC | 2901 |
| 4292 | GCTCCGCATCCCCGCGGGCC | 2902 |
| 4293 | CTCCGCATCCCCGCGGGCCG | 2903 |
| 4294 | TCCGCATCCCCGCGGGCCGG | 2904 |
| 4295 | CCGCATCCCCGCGGGCCGGC | 2905 |
| 4296 | CGCATCCCCGCGGGCCGGCG | 2906 |
| 4297 | GCATCCCCGCGGGCCGGCGC | 2907 |
| 4298 | CATCCCCGCGGGCCGGCGCT | 2908 |
| 4299 | ATCCCCGCGGGCCGGCGCTG | 2909 |
| 4300 | TCCCCGCGGGCCGGCGCTGG | 2910 |
| 4301 | CCCCGCGGGCCGGCGCTGGG | 2911 |
| 4302 | CCCGCGGGCCGGCGCTGGGC | 2912 |
| 4303 | CCGCGGGCCGGCGCTGGGCG | 2913 |
| 4304 | CGCGGGCCGGCGCTGGGCGG | 2914 |
| 4305 | GCGGGCCGGCGCTGGGCGGG | 2915 |
| 4306 | CGGGCCGGCGCTGGGCGGGG | 2916 |
| 4307 | GGGCCGGCGCTGGGCGGGGC | 2917 |
| 4308 | GGCCGGCGCTGGGCGGGGCC | 2918 |
| 4309 | GCCGGCGCTGGGCGGGGCCG | 2919 |
| 4310 | CCGGCGCTGGGCGGGGCCGG | 2920 |
| 4311 | CGGCGCTGGGCGGGGCCGGG | 2921 |
| 4312 | GGCGCTGGGCGGGGCCGGGC | 2922 |
| 4313 | GCGCTGGGCGGGGCCGGGCT | 2923 |
| 4314 | CGCTGGGCGGGGCCGGGCTG | 2924 |
| 4315 | GCTGGGCGGGGCCGGGCTGG | 2925 |
| 4316 | CTGGGCGGGGCCGGGCTGGA | 2926 |
| 4317 | TCACCGGGCCGCCGCGTCCG | 2837 |
| 4318 | CTCACCGGGCCGCCGCGTCC | 2836 |
| 4319 | ACTCACCGGGCCGCCGCGTC | 2835 |
| 4320 | GACTCACCGGGCCGCCGCGT | 2834 |
| 4321 | GGACTCACCGGGCCGCCGCG | 2833 |
| 4322 | GGGACTCACCGGGCCGCCGC | 2832 |
| 4323 | GGGGACTCACCGGGCCGCCG | 2831 |
| 4324 | CGGGGACTCACCGGGCCGCC | 2830 |
| 4325 | GCGGGGACTCACCGGGCCGC | 2829 |
| 4326 | GGCGGGGACTCACCGGGCCG | 2828 |
| 4327 | GGGCGGGGACTCACCGGGCC | 2827 |
| 4328 | CGGGCGGGGACTCACCGGGC | 2826 |
| 4329 | GCGGGCGGGGACTCACCGGG | 2825 |
| 4330 | GGCGGGCGGGGACTCACCGG | 2824 |
| 4331 | CGGCGGGCGGGGACTCACCG | 2823 |
| 4332 | ACGGCGGGCGGGGACTCACC | 2822 |
| 4333 | CACGGCGGGCGGGGACTCAC | 2821 |
| 4334 | CCACGGCGGGCGGGGACTCA | 2820 |
| 4335 | GCCACGGCGGGCGGGGACTC | 2819 |
| 4336 | GGCCACGGCGGGCGGGGACT | 2818 |
| 4337 | CGGCCACGGCGGGCGGGGAC | 2817 |
| 4338 | CACATCCGCCTCCGCCGCCCGG | 3160 |

```
Hot Zones (Relative upstream location to gene start site)
  1-350
  700-1100
1500-1650
1750-3650
```

### Examples

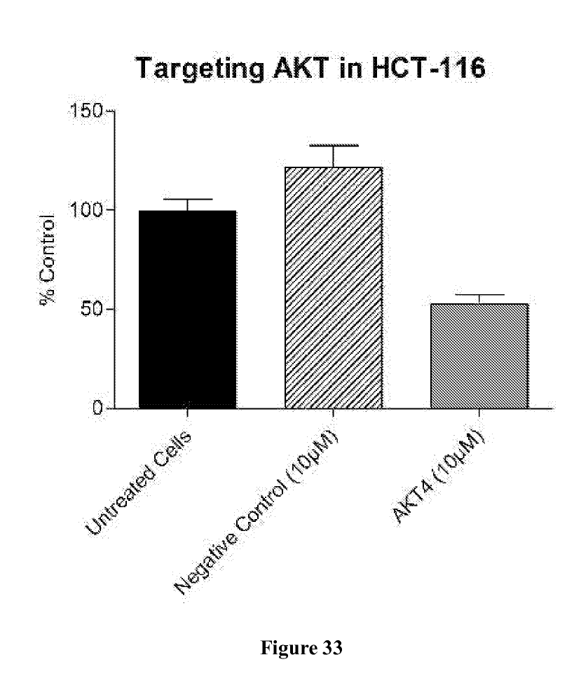

In FIG. 33, In MCF7 (human mammary breast cell line), AKT4 (169) produced statistically significant (P<0.05) inhibition at 10 μM compared to the untreated and negative control values. The AKT sequence AKT4 (169) fits the independent and dependent DNAi motif claims.

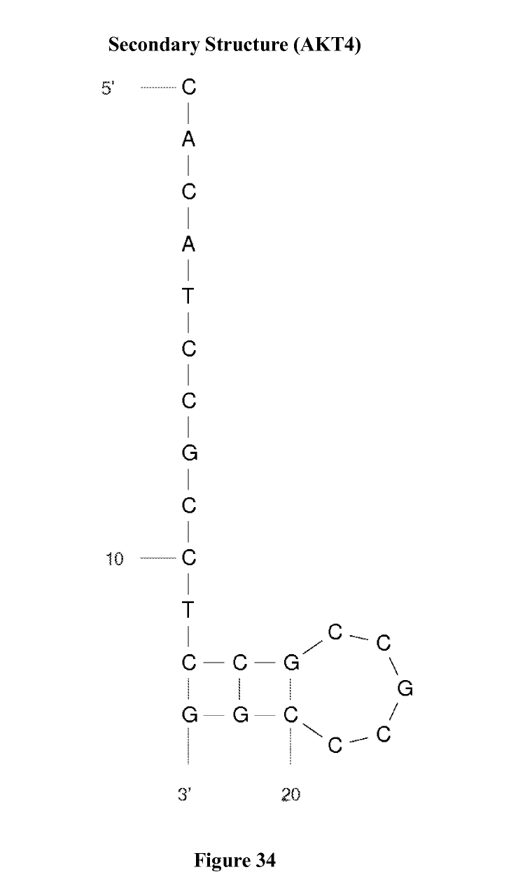

The secondary structure for AKT4 (169) is shown in FIG. 34.

Genetic Code (5′ Upstream Region)

(SEQ ID NO: 11965)

CGGCAGGACCGAGCGCGGCAGGCGGCTGGCCCAGCGCACGCAGCGCGGCC
CGAAGACGGGAGCAGGCGGCCGAGCACCGAGCGCTGGGCACCGGGCACCG
AGCGGCGGCGGCACGCGAGGCCCGGCCCCGAGCAGCGCCCCCGCCCGCCG
CGGCCTCCAGCCCGGCCCCGCCCAGCGCCGGCCCGCGGGGATGCGGAGCG
GCGGGCGCCGGAGGCCGCGGCCCGGCTAGGCCCGCGCTCGCGCCCGGACG
CGGCGGCCCGGTGAGTCCCCGCCCGCCGTGGCCGCCCGGGCCTGGATTTC
CTCCCCGCGGGCCGGGCCGCTTTGTTCGCGGCCGGTCGGGCCGGGGCGCG
AGCCGCGGCGCCGCCAGAATGGAGGAGCGGGAGCAGGAAGTGGCCGAGCG
GGCCTGGGCGGGGAGGGCGCGGGGCGCGCGGGCCCGGCCAAGGGAGGGCG
GCCCCACGCCGGGCGCCGGGGGTGCAGGCTGCCGGCCCCAGCCTCCCTCA
TGACCTTGGGGAGGCCGCTCCGCCGGGCGAGGCCGGGACCCGAGAGAAGG
GACGCCGCCGGGCCTGGAGCTGGGGCTCGGGGTGCCCCTGCGGGCCGTGG
GGCTCCCCGGGCGCTGGGCCAGCGAGGCAGCCGGCGGAGGTGCCCCGGGG
TTGGAGAAAGACTCGCCGCGGCCGGCCTTCAAGTTTGTGGGAGGGCCCCG
GAAGGAGACTTCGTTTCCCACGGACGAAAAGTTGTACGTGGTGGCGGGGT
ACCCAGGCTAGCCACAAAGGACTGTGACCCTCCTGGGCCCCGGAACTGCT
TCCTGTCTTGGGTGGGCCCTGGAGGTCCTGCCCGCCCATCCCAGAGGCCA
AGGCTTGGAGGGCAGCTGGGGCTTGCCCCTTAGATTGAGTATCCTGGGGC
GCTAGCGAGCTTGGTCCTGTCGGGACGGCCTCTGAGTGCTGCCTTGGTCA
GCGGGTGAGCTTGGGCCCCTGCTCTGCAGCCAGAGGCCGCCCCACATTCA
CTCCTGGGTCTCTCGGCCTTGCTCCAGGTGGCCACTTCTTGACTGCTTTG
AGTCCCTCATCCGAGCGAAGGGCGGACGGAGTCCGTTGGTGGGGGTCCGG
TTGCCTCTCCCGGGAGCTGTGTAGACTTCTCATACACCAGGGTTCTGGAG
GCAGATGGAGGAGCCCTTTCGAAAACAGAGTATTTTTTTTTAAGTTGTGA
CTTAATAATAGTAGCAAGAATATGTGCTTATGGTAAAGGCAGGCGGCAGG
TACGGAGGCTGTGGGAAGTCGGGGTCCCTCCGCCCCCACAGGCAGCCCTG
TGCTGGCCTGGTGTATACGTTTCTGTGCAGACGTACACCACCCTGTGTGA
GCACAGATGTATTTTTACACATGGCTCTGGACAGCTGTCTGACTCTGTCA
GCAGCAGGCCTTGGAGGGGCTCAGGCCCGTGTGGGGGTGGGGGGACATCC
AGAGGTCTTTGAGTCCAGCCCTCTGCCTCCAGGCCACGCCCACTCAGTGT
CGTCAGAGCCCCCTGTGCCTGAGGCGTGCGCGGCTCGGAGCCCTGCCCTC
GGAGTCCTGCGGTGCCTTCCTCGAGTCTGGCCTGCTTTCCATCCTGCTAA
GTACTTGGGGCATTTCCCTCTTTGGGTAAGGTGTGGTCTTCCCTGTCCTG
GCATTAGACACAAGGCAGTGGGCCTTCCTGCCATTCTAAGTGTAGCTTAA
GACAATCAGTGCAAAGCAACCCTTTGTGGGTGTCCAGCCCTTGCCTCGGG
AGGCCAGAAAGGTGGCCTGGGGGGAGAGCGTCTAAGCTGGCTGTGGAAAG
ACCCATGTTGGGATCCATTCCACAGAGGTCGTCAGGGGTCTCTGCCTGGC
CTGGAGGTCCCAGAGAGGACCCTCCTCCCCTCAGGAAGGCCCATCTGGAA
GGGTAGCAGAGGACTGCTCACAGGAAGAGCATGCGAAGTGCTCTTTCTGG
GGATGCCTGTAGTTGGTGATGTGGGAACTGGGTTTTGAGGGATGCCTAGG
AGTTCATCCATCAGAGGGGAAATGAGGAAGCCATGCAGGATCAATGGATA
AAGTGTGCTCAGGTGAGGGTTGGCTGGTGGGCCGCTGCAGGGCGGGGGCC
TGTCCAGTGCTCCCCCACTTACTTGCTGCCTCCCGACTGCTGTAATTATG
GGTCTGTAACCACCCTGGACTGGGTGCTCCTCACTGACGGACTTGTCTGA
ACCTCTCTTTGTCTCCAGCGCCCAGCACTGGGCCTGGCAAAACCTGAGAC
GCCCGGTACATGTTGGCCAAATGAATGAACCAGATTCAGACCGGCAGGGG
CGCTGTGGTTTAGGAGGGGCCTGGGGTTTCTCCCAGGAGGTTTTTGGGCT
TGCGCTGGAGGGCTCTGGACTCCCGTTTGCGCCAGTGGCCTGCATCCTGG
TCCTGTCTTCCTCATGTTTGAATTTCTTTGCTTTCCTAGTCTGGGGAGCA
GGGAGGAGCCCTGTGCCCTGTCCCAGGATCCATGGGTAGGAACACCATGG
ACAGGGAGAGCAAACGGGGCCATCTGTCACCAGGGGCTTAGGGAAGGCCG
AGCCAGCCTGGGTCAAAGAAGTCAAAGGGGCTGCCTGGAGGAGGCAGCCT
GTCAGCTGGTGCATCAGGTTAGGGAGGCTGGGAAGGCCTTTTGGGGATGG
GGGTGATTTGTCCAACGGCTGGGGGAGGTGGGAATGGGGAGGTGAGCAAG
GCAGCAGCTCTCAGGGCCTGGCTGTTGCGGGTGGTGGTGGCAGGGGCTGG
AGGCTCTAAGCCTAGAATAAGGAGAGGCCCAGGTCCAGGGAACTGTGTTC
AATTACATGGATTTGACACTTGGCAGCCCTGAGTGTTTTGGGGAGAGGGA
AGGCAGGCGGGCAGATGGGGGTCAGAGAGCTTAGAGGGATGGCAGCCCAC
CTGGGAAGGCAGGTGCGGGTGGAGCCCCCAGGCACGTGCAGTGGGTCTCT
GGCTCACCCAGGGCGAGGAGCTGCCCTTAGCCAGGCGTGGCCTCACATTC
AGCTTCCTTTGCTTCTCCCAGAGGCTGTGGCCAGGCCAGCTGGGCTCGGG
GAGCGCCAGCCTGAGAGGAGCGCGTGAGCGTCGCGGGAGCCTCGGGCACC
ATG

17) CRAF. RAF proto-oncogene serine/threonine-protein kinase also known as proto-oncogene c-RAF or simply c-Raf or even Raf-1 is an enzyme is encoded by the RAF1 gene. The c-Raf protein is part of the ERK1/2 pathway as a MAP kinase kinase kinase (MAP3K) that functions downstream of the Ras subfamily of membrane associated GTPases.

Elevated C-Raf mRNA or protein levels have been identified in AML, head and neck cancer, prostate cancer and ovarian cancer (Schmidt et al., Leuk Res. 1994; 18:409-13, Riva et al., Eur J Cancer B Oral Oncol. 1995; 31B:384-91, Muhkerjhee et al., Prostate. 2005; 64:101-7). In ovarian cancer cell lines, antisense oligodeoxynucleotides (ODNs) inhibited cell proliferation in vitro (McPhillips et al., Br J Cancer. 2001; 85:1753-8) with similar results seen in lines derived from lung, cervical, prostate and colon carcinomas showed the same phenomenon.

Inhibiting cRAF may be useful against diabetic retinopathy, one of the leading causes of blindness A c-RAF inhibitor (iCo-007) is being developed for the treatment of various eye diseases that occur as complications of diabetes. In patients with diffuse diabetic macular edema presented positive results from the Phase 1 study showing that subjects tolerated iCo-007 well. In this study, a number of individuals exhibited a decrease of central macular edema compared to baseline using an analytical method called optical coherence tomography prompting the initiation of a Phase 2 study on iCo-007 in patients with diabetic macular edema.

Hereditary gain-of-function mutations of c-Raf are implicated in some rare, but severe syndromes. Mutation of c-Raf is one of the possible causes of Noonan syndrome: affected individuals have congenital heart defects, short and dysmorhic stature and several other deformities. Similar mutations in c-Raf can also cause a related condition, termed LEOPARD syndrome (Lentigo, Electrocardiographic abnormalities, Ocular hypertelorism, Pulmonary stenosis, Abnormal genitalia, Retarded growth, Deafness), with a complex association of defects.

Protein: c-Raf Gene: RAF1 (Homo sapiens, chromosome 3, 12625100-12705700 [NCBI Reference Sequence: NC_{—}000003.11]; start site location: 12660220; strand: negative)

Gene Identification

| | |
|---|---|
| GeneID | 5894 |
| HGNC | 9829 |
| HPRD | 01265 |
| MIM | 164760 |

Targeted Sequences

| | | Relative |
|---|---|---|
| | | upstream |
| | | location |
| Sequence | | to gene |
| ID No: | Sequence (5′-3′) | start site |
| 4339 | GCGCGAGCCCTACTGGCAGTCG | 25996 |
| 4462 | CGGGGCGTGGCCTAGCGATCTGGTGGCCG | 26073 |
| 4517 | TTTCGAAGCTGAAGAGGTTAGGCGACG | 26106 |
| 4519 | CGACGCTGACTTGCTTTCAGGAG | 26127 |
| 4533 | AATCGAGAAGAACCGGCTTTCGG | 26161 |
| 4556 | CTTTGACGCGTCCTCTCCGGGC | 26295 |
| 4585 | CGGCTCCGCCACTTGACAGCTATGTGG | 26334 |
| 4605 | AGGCGGAGATTGCGGTGAGCCGAAATCGCG | 27188 |
| 4609 | AGGCCGCCCCAACGTCCTGTCGTTCGGCGG | 25618 |
| 4677 | TCTCGCCCGCTCCTCCTCCCCGCGGCGGGTG | 25653 |
| 4745 | CGGGAGGCGGTCACATTCGGCGCG | 25690 |
| 4782 | CGGAGCCCCGAGCAGCCCCCGCATCG | 25730 |
| 4871 | CGCGCTCCGCGCCTCAGGGCACGCGCC | 25763 |
| 4960 | AGCCGTTCCCGCCTCACAATCG | 25840 |
| 4984 | CCGCCATCTAAGATGGCGGCC | 25876 |
| 5047 | CGGGCGGCCCAGACGAGCGAGCCCTCG | 25920 |
| 5110 | CGTCCTCCCGACCTGCGACGCCACCGGC | 25957 |

Target Shift Sequences

| | | Relative |
|---|---|---|
| | | upstream |
| | | location |
| Sequence | | to gene |
| ID No: | Sequence (5′-3′) | start site |
| 4339 | GCGCGAGCCCTACTGGCAGTCG | 25996 |
| 4340 | CGCGAGCCCTACTGGCAGTC | 25997 |
| 4341 | GCGAGCCCTACTGGCAGTCG | 25998 |
| 4342 | CGAGCCCTACTGGCAGTCGA | 25999 |
| 4343 | GAGCCCTACTGGCAGTCGAC | 26000 |
| 4344 | AGCCCTACTGGCAGTCGACT | 26001 |
| 4345 | GCCCTACTGGCAGTCGACTT | 26002 |
| 4346 | CCCTACTGGCAGTCGACTTC | 26003 |
| 4347 | CCTACTGGCAGTCGACTTCT | 26004 |
| 4348 | CTACTGGCAGTCGACTTCTA | 26005 |
| 4349 | TACTGGCAGTCGACTTCTAA | 26006 |
| 4350 | ACTGGCAGTCGACTTCTAAC | 26007 |
| 4351 | CTGGCAGTCGACTTCTAACT | 26008 |
| 4352 | TGGCAGTCGACTTCTAACTT | 26009 |
| 4353 | GGCAGTCGACTTCTAACTTG | 26010 |
| 4354 | GCAGTCGACTTCTAACTTGG | 26011 |
| 4355 | CAGTCGACTTCTAACTTGGC | 26012 |
| 4356 | AGTCGACTTCTAACTTGGCT | 26013 |
| 4357 | GTCGACTTCTAACTTGGCTC | 26014 |
| 4358 | TCGACTTCTAACTTGGCTCG | 26015 |
| 4359 | CGACTTCTAACTTGGCTCGG | 26016 |
| 4360 | GACTTCTAACTTGGCTCGGG | 26017 |
| 4361 | ACTTCTAACTTGGCTCGGGC | 26018 |
| 4362 | CTTCTAACTTGGCTCGGGCA | 26019 |
| 4363 | TTCTAACTTGGCTCGGGCAT | 26020 |
| 4364 | TCTAACTTGGCTCGGGCATC | 26021 |
| 4365 | CTAACTTGGCTCGGGCATCC | 26022 |
| 4366 | TAACTTGGCTCGGGCATCCA | 26023 |
| 4367 | AACTTGGCTCGGGCATCCAT | 26024 |
| 4368 | ACTTGGCTCGGGCATCCATC | 26025 |
| 4369 | CTTGGCTCGGGCATCCATCG | 26026 |
| 4370 | TTGGCTCGGGCATCCATCGC | 26027 |
| 4371 | TGGCTCGGGCATCCATCGCT | 26028 |
| 4372 | GGCTCGGGCATCCATCGCTC | 26029 |
| 4373 | GCTCGGGCATCCATCGCTCT | 26030 |
| 4374 | CTCGGGCATCCATCGCTCTG | 26031 |
| 4375 | TCGGGCATCCATCGCTCTGG | 26032 |
| 4376 | CGGGCATCCATCGCTCTGGC | 26033 |
| 4377 | GGGCATCCATCGCTCTGGCC | 26034 |
| 4378 | GGCATCCATCGCTCTGGCCT | 26035 |
| 4379 | GCATCCATCGCTCTGGCCTG | 26036 |
| 4380 | CATCCATCGCTCTGGCCTGA | 26037 |
| 4381 | ATCCATCGCTCTGGCCTGAA | 26038 |
| 4382 | TCCATCGCTCTGGCCTGAAC | 26039 |
| 4383 | CCATCGCTCTGGCCTGAACT | 26040 |
| 4384 | CATCGCTCTGGCCTGAACTC | 26041 |
| 4385 | ATCGCTCTGGCCTGAACTCA | 26042 |
| 4386 | TCGCTCTGGCCTGAACTCAG | 26043 |
| 4387 | CGCTCTGGCCTGAACTCAGG | 26044 |
| 4388 | TGCGCGAGCCCTACTGGCAG | 25995 |
| 4389 | CTGCGCGAGCCCTACTGGCA | 25994 |
| 4390 | TCTGCGCGAGCCCTACTGGC | 25993 |
| 4391 | TTCTGCGCGAGCCCTACTGG | 25992 |
| 4392 | ATTCTGCGCGAGCCCTACTG | 25991 |
| 4393 | GATTCTGCGCGAGCCCTACT | 25990 |
| 4394 | CGATTCTGCGCGAGCCCTAC | 25989 |
| 4395 | CCGATTCTGCGCGAGCCCTA | 25988 |
| 4396 | TCCGATTCTGCGCGAGCCCT | 25987 |
| 4397 | CTCCGATTCTGCGCGAGCCC | 25986 |
| 4398 | TCTCCGATTCTGCGCGAGCC | 25985 |
| 4399 | CTCTCCGATTCTGCGCGAGC | 25984 |
| 4400 | GCTCTCCGATTCTGCGCGAG | 25983 |
| 4401 | GGCTCTCCGATTCTGCGCGA | 25982 |
| 4402 | CGGCTCTCCGATTCTGCGCG | 25981 |
| 4403 | CCGGCTCTCCGATTCTGCGC | 25980 |
| 4404 | ACCGGCTCTCCGATTCTGCG | 25979 |
| 4405 | CACCGGCTCTCCGATTCTGC | 25978 |
| 4406 | CCACCGGCTCTCCGATTCTG | 25977 |
| 4407 | GCCACCGGCTCTCCGATTCT | 25976 |
| 4408 | CGCCACCGGCTCTCCGATTC | 25975 |
| 4409 | ACGCCACCGGCTCTCCGATT | 25974 |
| 4410 | GACGCCACCGGCTCTCCGAT | 25973 |
| 4411 | CGACGCCACCGGCTCTCCGA | 25972 |
| 4412 | GCGACGCCACCGGCTCTCCG | 25971 |
| 4413 | TGCGACGCCACCGGCTCTCC | 25970 |
| 4414 | CTGCGACGCCACCGGCTCTC | 25969 |
| 4415 | CCTGCGACGCCACCGGCTCT | 25968 |
| 4416 | ACCTGCGACGCCACCGGCTC | 25967 |
| 4417 | GACCTGCGACGCCACCGGCT | 25966 |
| 4418 | CGACCTGCGACGCCACCGGC | 25965 |
| 4419 | CCGACCTGCGACGCCACCGG | 25964 |
| 4420 | CCCGACCTGCGACGCCACCG | 25963 |
| 4421 | TCCCGACCTGCGACGCCACC | 25962 |
| 4422 | CTCCCGACCTGCGACGCCAC | 25961 |
| 4423 | CCTCCCGACCTGCGACGCCA | 25960 |
| 4424 | TCCTCCCGACCTGCGACGCC | 25959 |
| 4425 | GTCCTCCCGACCTGCGACGC | 25958 |
| 4426 | CGTCCTCCCGACCTGCGACG | 25957 |
| 4427 | TCGTCCTCCCGACCTGCGAC | 25956 |
| 4428 | CTCGTCCTCCCGACCTGCGA | 25955 |
| 4429 | GCTCGTCCTCCCGACCTGCG | 25954 |
| 4430 | TGCTCGTCCTCCCGACCTGC | 25953 |
| 4431 | GTGCTCGTCCTCCCGACCTG | 25952 |
| 4432 | GGTGCTCGTCCTCCCGACCT | 25951 |
| 4433 | CGGTGCTCGTCCTCCCGACC | 25950 |
| 4434 | TCGGTGCTCGTCCTCCCGAC | 25949 |
| 4435 | CTCGGTGCTCGTCCTCCCGA | 25948 |
| 4436 | ACTCGGTGCTCGTCCTCCCG | 25947 |
| 4437 | GACTCGGTGCTCGTCCTCCC | 25946 |
| 4438 | CGACTCGGTGCTCGTCCTCC | 25945 |
| 4439 | TCGACTCGGTGCTCGTCCTC | 25944 |
| 4440 | CTCGACTCGGTGCTCGTCCT | 25943 |
| 4441 | CCTCGACTCGGTGCTCGTCC | 25942 |
| 4442 | CCCTCGACTCGGTGCTCGTC | 25941 |
| 4443 | GCCCTCGACTCGGTGCTCGT | 25940 |
| 4444 | AGCCCTCGACTCGGTGCTCG | 25939 |
| 4445 | GAGCCCTCGACTCGGTGCTC | 25938 |
| 4446 | CGAGCCCTCGACTCGGTGCT | 25937 |
| 4447 | GCGAGCCCTCGACTCGGTGC | 25936 |
| 4448 | AGCGAGCCCTCGACTCGGTG | 25935 |
| 4449 | GAGCGAGCCCTCGACTCGGT | 25934 |
| 4450 | CGAGCGAGCCCTCGACTCGG | 25933 |
| 4451 | ACGAGCGAGCCCTCGACTCG | 25932 |
| 4452 | GACGAGCGAGCCCTCGACTC | 25931 |
| 4453 | AGACGAGCGAGCCCTCGACT | 25930 |
| 4454 | CAGACGAGCGAGCCCTCGAC | 25929 |
| 4455 | CCAGACGAGCGAGCCCTCGA | 25928 |
| 4456 | CCCAGACGAGCGAGCCCTCG | 25927 |
| 4457 | GCCCAGACGAGCGAGCCCTC | 25926 |
| 4458 | GGCCCAGACGAGCGAGCCCT | 25925 |
| 4459 | CGGCCCAGACGAGCGAGCCC | 25924 |
| 4460 | GCGGCCCAGACGAGCGAGCC | 25923 |
| 4461 | GGCGGCCCAGACGAGCGAGC | 25922 |
| 4462 | CGGGGCGTGGCCTAGCGATCTGGTGGCCG | 26073 |
| 4463 | GGGGCGTGGCCTAGCGATCT | 26074 |
| 4464 | GGGCGTGGCCTAGCGATCTG | 26075 |
| 4465 | GGCGTGGCCTAGCGATCTGG | 26076 |
| 4466 | GCGTGGCCTAGCGATCTGGT | 26077 |
| 4467 | CGTGGCCTAGCGATCTGGTG | 26078 |
| 4468 | GTGGCCTAGCGATCTGGTGG | 26079 |
| 4469 | TGGCCTAGCGATCTGGTGGC | 26080 |
| 4470 | GGCCTAGCGATCTGGTGGCC | 26081 |
| 4471 | GCCTAGCGATCTGGTGGCCG | 26082 |
| 4472 | CCTAGCGATCTGGTGGCCGC | 26083 |
| 4473 | CTAGCGATCTGGTGGCCGCC | 26084 |
| 4474 | TAGCGATCTGGTGGCCGCCA | 26085 |
| 4475 | AGCGATCTGGTGGCCGCCAT | 26086 |
| 4476 | GCGATCTGGTGGCCGCCATT | 26087 |
| 4477 | CGATCTGGTGGCCGCCATTT | 26088 |
| 4478 | GATCTGGTGGCCGCCATTTC | 26089 |
| 4479 | ATCTGGTGGCCGCCATTTCG | 26090 |
| 4480 | TCTGGTGGCCGCCATTTCGA | 26091 |
| 4481 | CTGGTGGCCGCCATTTCGAA | 26092 |
| 4482 | TGGTGGCCGCCATTTCGAAG | 26093 |
| 4483 | GGTGGCCGCCATTTCGAAGC | 26094 |
| 4484 | GTGGCCGCCATTTCGAAGCT | 26095 |
| 4485 | TGGCCGCCATTTCGAAGCTG | 26096 |
| 4486 | GGCCGCCATTTCGAAGCTGA | 26097 |
| 4487 | GCCGCCATTTCGAAGCTGAA | 26098 |
| 4488 | CCGCCATTTCGAAGCTGAAG | 26099 |
| 4489 | CGCCATTTCGAAGCTGAAGA | 26100 |
| 4490 | GCCATTTCGAAGCTGAAGAG | 26101 |
| 4491 | CCATTTCGAAGCTGAAGAGG | 26102 |
| 4492 | CATTTCGAAGCTGAAGAGGT | 26103 |
| 4493 | CCGGGGCGTGGCCTAGCGAT | 26072 |
| 4494 | CCCGGGGCGTGGCCTAGCGA | 26071 |
| 4495 | CCCCGGGGCGTGGCCTAGCG | 26070 |
| 4496 | CCCCCGGGGCGTGGCCTAGC | 26069 |
| 4497 | GCCCCCGGGGCGTGGCCTAG | 26068 |
| 4498 | CGCCCCCGGGGCGTGGCCTA | 26067 |
| 4499 | CCGCCCCCGGGGCGTGGCCT | 26066 |
| 4500 | CCCGCCCCCGGGGCGTGGCC | 26065 |
| 4501 | CCCCGCCCCCGGGGCGTGGC | 26064 |
| 4502 | GCCCCGCCCCCGGGGCGTGG | 26063 |
| 4503 | GGCCCCGCCCCCGGGGCGTG | 26062 |
| 4504 | AGGCCCCGCCCCCGGGGCGT | 26061 |
| 4505 | CAGGCCCCGCCCCCGGGGCG | 26060 |
| 4506 | TCAGGCCCCGCCCCCGGGGC | 26059 |
| 4507 | CTCAGGCCCCGCCCCCGGGG | 26058 |
| 4508 | ACTCAGGCCCCGCCCCCGGG | 26057 |
| 4509 | AACTCAGGCCCCGCCCCCGG | 26056 |
| 4510 | GAACTCAGGCCCCGCCCCCG | 26055 |
| 4511 | TGAACTCAGGCCCCGCCCCC | 26054 |
| 4512 | CTGAACTCAGGCCCCGCCCC | 26053 |
| 4513 | CCTGAACTCAGGCCCCGCCC | 26052 |
| 4514 | GCCTGAACTCAGGCCCCGCC | 26051 |
| 4515 | GGCCTGAACTCAGGCCCCGC | 26050 |
| 4516 | TGGCCTGAACTCAGGCCCCG | 26049 |
| 4517 | TTTCGAAGCTGAAGAGGTTAGGCGACG | 26105 |
| 4518 | TTCGAAGCTGAAGAGGTTAG | 26106 |
| 4519 | CGACGCTGACTTGCTTTCAGGAG | 26127 |
| 4520 | GACGCTGACTTGCTTTCAGG | 26128 |
| 4521 | ACGCTGACTTGCTTTCAGGA | 26129 |
| 4522 | CGCTGACTTGCTTTCAGGAG | 26130 |
| 4523 | GCGACGCTGACTTGCTTTCA | 26126 |
| 4524 | GGCGACGCTGACTTGCTTTC | 26125 |
| 4525 | AGGCGACGCTGACTTGCTTT | 26124 |
| 4526 | TAGGCGACGCTGACTTGCTT | 26123 |
| 4527 | TTAGGCGACGCTGACTTGCT | 26122 |
| 4528 | GTTAGGCGACGCTGACTTGC | 26121 |
| 4529 | GGTTAGGCGACGCTGACTTG | 26120 |
| 4530 | AGGTTAGGCGACGCTGACTT | 26119 |
| 4531 | GAGGTTAGGCGACGCTGACT | 26118 |
| 4532 | AGAGGTTAGGCGACGCTGAC | 26117 |
| 4533 | AATCGAGAAGAACCGGCTTTCGG | 26161 |
| 4534 | ATCGAGAAGAACCGGCTTTC | 26162 |
| 4535 | TCGAGAAGAACCGGCTTTCG | 26163 |
| 4536 | CGAGAAGAACCGGCTTTCGG | 26164 |
| 4537 | GAGAAGAACCGGCTTTCGGC | 26165 |
| 4538 | AGAAGAACCGGCTTTCGGCC | 26166 |
| 4539 | GAAGAACCGGCTTTCGGCCA | 26167 |
| 4540 | AAGAACCGGCTTTCGGCCAG | 26168 |
| 4541 | AGAACCGGCTTTCGGCCAGC | 26169 |
| 4542 | GAACCGGCTTTCGGCCAGCC | 26170 |
| 4543 | AACCGGCTTTCGGCCAGCCA | 26171 |
| 4544 | ACCGGCTTTCGGCCAGCCAG | 26172 |
| 4545 | CCGGCTTTCGGCCAGCCAGG | 26173 |
| 4546 | CGGCTTTCGGCCAGCCAGGA | 26174 |
| 4547 | GGCTTTCGGCCAGCCAGGAG | 26175 |
| 4548 | GCTTTCGGCCAGCCAGGAGT | 26176 |
| 4549 | CTTTCGGCCAGCCAGGAGTG | 26177 |
| 4550 | TTTCGGCCAGCCAGGAGTGG | 26178 |
| 4551 | TTCGGCCAGCCAGGAGTGGC | 26179 |
| 4552 | TCGGCCAGCCAGGAGTGGCC | 26180 |
| 4553 | CGGCCAGCCAGGAGTGGCCA | 26181 |
| 4554 | TAATCGAGAAGAACCGGCTT | 26160 |
| 4555 | GTAATCGAGAAGAACCGGCT | 26159 |
| 4556 | CTTTGACGCGTCCTCTCCGGGC | 26295 |
| 4557 | TTTGACGCGTCCTCTCCGGG | 26296 |
| 4558 | TTGACGCGTCCTCTCCGGGC | 26297 |
| 4559 | TGACGCGTCCTCTCCGGGCA | 26298 |
| 4560 | GACGCGTCCTCTCCGGGCAC | 26299 |
| 4561 | ACGCGTCCTCTCCGGGCACT | 26300 |
| 4562 | CGCGTCCTCTCCGGGCACTT | 26301 |
| 4563 | GCGTCCTCTCCGGGCACTTT | 26302 |
| 4564 | CGTCCTCTCCGGGCACTTTA | 26303 |
| 4565 | GTCCTCTCCGGGCACTTTAA | 26304 |
| 4566 | TCCTCTCCGGGCACTTTAAT | 26305 |
| 4567 | CCTCTCCGGGCACTTTAATA | 26306 |
| 4568 | CTCTCCGGGCACTTTAATAC | 26307 |
| 4569 | TCTCCGGGCACTTTAATACC | 26308 |
| 4570 | CTCCGGGCACTTTAATACCA | 26309 |
| 4571 | TCCGGGCACTTTAATACCAA | 26310 |
| 4572 | CCGGGCACTTTAATACCAAA | 26311 |
| 4573 | ACTTTGACGCGTCCTCTCCG | 26294 |
| 4574 | AACTTTGACGCGTCCTCTCC | 26293 |
| 4575 | CAACTTTGACGCGTCCTCTC | 26292 |
| 4576 | CCAACTTTGACGCGTCCTCT | 26291 |
| 4577 | TCCAACTTTGACGCGTCCTC | 26290 |
| 4578 | GTCCAACTTTGACGCGTCCT | 26289 |
| 4579 | TGTCCAACTTTGACGCGTCC | 26288 |
| 4580 | GTGTCCAACTTTGACGCGTC | 26287 |
| 4581 | AGTGTCCAACTTTGACGCGT | 26286 |
| 4582 | CAGTGTCCAACTTTGACGCG | 26285 |
| 4583 | ACAGTGTCCAACTTTGACGC | 26284 |
| 4584 | CACAGTGTCCAACTTTGACG | 26283 |
| 4585 | CGGCTCCGCCACTTGACAGCTATGTGG | 26334 |
| 4586 | GGCTCCGCCACTTGACAGCT | 26335 |
| 4587 | GCTCCGCCACTTGACAGCTA | 26336 |
| 4588 | CTCCGCCACTTGACAGCTAT | 26337 |
| 4589 | TCCGCCACTTGACAGCTATG | 26338 |
| 4590 | CCGCCACTTGACAGCTATGT | 26339 |
| 4591 | CGCCACTTGACAGCTATGTG | 26340 |
| 4592 | ACGGCTCCGCCACTTGACAG | 26333 |
| 4593 | CACGGCTCCGCCACTTGACA | 26332 |
| 4594 | TCACGGCTCCGCCACTTGAC | 26331 |
| 4595 | ATCACGGCTCCGCCACTTGA | 26330 |
| 4596 | AATCACGGCTCCGCCACTTG | 26329 |
| 4597 | AAATCACGGCTCCGCCACTT | 26328 |
| 4598 | CAAATCACGGCTCCGCCACT | 26327 |
| 4599 | CCAAATCACGGCTCCGCCAC | 26326 |
| 4600 | ACCAAATCACGGCTCCGCCA | 26325 |
| 4601 | TACCAAATCACGGCTCCGCC | 26324 |
| 4602 | ATACCAAATCACGGCTCCGC | 26323 |
| 4603 | AATACCAAATCACGGCTCCG | 26322 |
| 4604 | TAATACCAAATCACGGCTCC | 26321 |
| 4605 | AGGCGGAGATTGCGGTGAGCCGAAATCGCG | 27188 |
| 4606 | GGCGGAGATTGCGGTGAGCC | 27189 |
| 4607 | GCGGAGATTGCGGTGAGCCG | 27190 |
| 4608 | CGGAGATTGCGGTGAGCCGA | 27191 |
| 4609 | AGGCCGCCCCAACGTCCTGTCGTTCGGCGG | 25618 |
| 4610 | GGCCGCCCCAACGTCCTGTC | 25619 |
| 4611 | GCCGCCCCAACGTCCTGTCG | 25620 |
| 4612 | CCGCCCCAACGTCCTGTCGT | 25621 |
| 4613 | CGCCCCAACGTCCTGTCGTT | 25622 |
| 4614 | GCCCCAACGTCCTGTCGTTC | 25623 |
| 4615 | CCCCAACGTCCTGTCGTTCG | 25624 |
| 4616 | CCCAACGTCCTGTCGTTCGG | 25625 |
| 4617 | CCAACGTCCTGTCGTTCGGC | 25626 |
| 4618 | CAACGTCCTGTCGTTCGGCG | 25627 |
| 4619 | AACGTCCTGTCGTTCGGCGG | 25628 |
| 4620 | ACGTCCTGTCGTTCGGCGGC | 25629 |
| 4621 | CGTCCTGTCGTTCGGCGGCA | 25630 |
| 4622 | GTCCTGTCGTTCGGCGGCAG | 25631 |
| 4623 | TCCTGTCGTTCGGCGGCAGC | 25632 |
| 4624 | CCTGTCGTTCGGCGGCAGCT | 25633 |
| 4625 | CTGTCGTTCGGCGGCAGCTT | 25634 |
| 4626 | TGTCGTTCGGCGGCAGCTTC | 25635 |
| 4627 | GTCGTTCGGCGGCAGCTTCT | 25636 |
| 4628 | TCGTTCGGCGGCAGCTTCTC | 25637 |
| 4629 | CGTTCGGCGGCAGCTTCTCG | 25638 |
| 4630 | GTTCGGCGGCAGCTTCTCGC | 25639 |
| 4631 | TTCGGCGGCAGCTTCTCGCC | 25640 |
| 4632 | TCGGCGGCAGCTTCTCGCCC | 25641 |
| 4633 | CGGCGGCAGCTTCTCGCCCG | 25642 |
| 4634 | GGCGGCAGCTTCTCGCCCGC | 25643 |
| 4635 | GCGGCAGCTTCTCGCCCGCT | 25644 |
| 4636 | CGGCAGCTTCTCGCCCGCTC | 25645 |
| 4637 | GGCAGCTTCTCGCCCGCTCC | 25646 |
| 4638 | GCAGCTTCTCGCCCGCTCCT | 25647 |
| 4639 | CAGCTTCTCGCCCGCTCCTC | 25648 |
| 4640 | AGCTTCTCGCCCGCTCCTCC | 25649 |
| 4641 | GCTTCTCGCCCGCTCCTCCT | 25650 |
| 4642 | CTTCTCGCCCGCTCCTCCTC | 25651 |
| 4643 | TTCTCGCCCGCTCCTCCTCC | 25652 |
| 4644 | TCTCGCCCGCTCCTCCTCCC | 25653 |
| 4645 | CTCGCCCGCTCCTCCTCCCC | 25654 |
| 4646 | TCGCCCGCTCCTCCTCCCCG | 25655 |
| 4647 | CGCCCGCTCCTCCTCCCCGC | 25656 |
| 4648 | GCCCGCTCCTCCTCCCCGCG | 25657 |
| 4649 | CCCGCTCCTCCTCCCCGCGG | 25658 |
| 4650 | CCGCTCCTCCTCCCCGCGGC | 25659 |
| 4651 | CGCTCCTCCTCCCCGCGGCG | 25660 |
| 4652 | GCTCCTCCTCCCCGCGGCGG | 25661 |
| 4653 | CTCCTCCTCCCCGCGGCGGG | 25662 |
| 4654 | TCCTCCTCCCCGCGGCGGGT | 25663 |
| 4655 | CCTCCTCCCCGCGGCGGGTG | 25664 |
| 4656 | CTCCTCCCCGCGGCGGGTGA | 25665 |
| 4657 | TCCTCCCCGCGGCGGGTGAG | 25666 |
| 4658 | CCTCCCCGCGGCGGGTGAGG | 25667 |
| 4659 | CTCCCCGCGGCGGGTGAGGG | 25668 |
| 4660 | TCCCCGCGGCGGGTGAGGGA | 25669 |
| 4661 | CCCCGCGGCGGGTGAGGGAG | 25670 |
| 4662 | CCCGCGGCGGGTGAGGGAGC | 25671 |
| 4663 | CAGGCCGCCCCAACGTCCTG | 25617 |
| 4664 | CCAGGCCGCCCCAACGTCCT | 25616 |
| 4665 | GCCAGGCCGCCCCAACGTCC | 25615 |
| 4666 | AGCCAGGCCGCCCCAACGTC | 25614 |
| 4667 | GAGCCAGGCCGCCCCAACGT | 25613 |
| 4668 | GGAGCCAGGCCGCCCCAACG | 25612 |
| 4669 | GGGAGCCAGGCCGCCCCAAC | 25611 |
| 4670 | AGGGAGCCAGGCCGCCCCAA | 25610 |
| 4671 | GAGGGAGCCAGGCCGCCCCA | 25609 |
| 4672 | TGAGGGAGCCAGGCCGCCCC | 25608 |
| 4673 | CTGAGGGAGCCAGGCCGCCC | 25607 |
| 4674 | CCTGAGGGAGCCAGGCCGCC | 25606 |
| 4675 | ACCTGAGGGAGCCAGGCCGC | 25605 |
| 4676 | TACCTGAGGGAGCCAGGCCG | 25604 |
| 4677 | TCTCGCCCGCTCCTCCTCCCCGCGGCGGGTG | 25653 |
| 4678 | CTCGCCCGCTCCTCCTCCCC | 25654 |
| 4679 | TCGCCCGCTCCTCCTCCCCG | 25655 |
| 4680 | CGCCCGCTCCTCCTCCCCGC | 25656 |
| 4681 | GCCCGCTCCTCCTCCCCGCG | 25657 |
| 4682 | CCCGCTCCTCCTCCCCGCGG | 25658 |
| 4683 | CCGCTCCTCCTCCCCGCGGC | 25659 |
| 4684 | CGCTCCTCCTCCCCGCGGCG | 25660 |
| 4685 | GCTCCTCCTCCCCGCGGCGG | 25661 |
| 4686 | CTCCTCCTCCCCGCGGCGGG | 25662 |
| 4687 | TCCTCCTCCCCGCGGCGGGT | 25663 |
| 4688 | CCTCCTCCCCGCGGCGGGTG | 25664 |
| 4689 | CTCCTCCCCGCGGCGGGTGA | 25665 |
| 4690 | TCCTCCCCGCGGCGGGTGAG | 25666 |
| 4691 | CCTCCCCGCGGCGGGTGAGG | 25667 |
| 4692 | CTCCCCGCGGCGGGTGAGGG | 25668 |
| 4693 | TCCCCGCGGCGGGTGAGGGA | 25669 |
| 4694 | CCCCGCGGCGGGTGAGGGAG | 25670 |
| 4695 | CCCGCGGCGGGTGAGGGAGC | 25671 |
| 4696 | TTCTCGCCCGCTCCTCCTCC | 25652 |
| 4697 | CTTCTCGCCCGCTCCTCCTC | 25651 |
| 4698 | GCTTCTCGCCCGCTCCTCCT | 25650 |
| 4699 | AGCTTCTCGCCCGCTCCTCC | 25649 |
| 4700 | CAGCTTCTCGCCCGCTCCTC | 25648 |
| 4701 | GCAGCTTCTCGCCCGCTCCT | 25647 |
| 4702 | GGCAGCTTCTCGCCCGCTCC | 25646 |
| 4703 | CGGCAGCTTCTCGCCCGCTC | 25645 |
| 4704 | GCGGCAGCTTCTCGCCCGCT | 25644 |
| 4705 | GGCGGCAGCTTCTCGCCCGC | 25643 |
| 4706 | CGGCGGCAGCTTCTCGCCCG | 25642 |
| 4707 | TCGGCGGCAGCTTCTCGCCC | 25641 |
| 4708 | TTCGGCGGCAGCTTCTCGCC | 25640 |
| 4709 | GTTCGGCGGCAGCTTCTCGC | 25639 |
| 4710 | CGTTCGGCGGCAGCTTCTCG | 25638 |
| 4711 | TCGTTCGGCGGCAGCTTCTC | 25637 |
| 4712 | GTCGTTCGGCGGCAGCTTCT | 25636 |
| 4713 | TGTCGTTCGGCGGCAGCTTC | 25635 |
| 4714 | CTGTCGTTCGGCGGCAGCTT | 25634 |
| 4715 | CCTGTCGTTCGGCGGCAGCT | 25633 |
| 4716 | TCCTGTCGTTCGGCGGCAGC | 25632 |
| 4717 | GTCCTGTCGTTCGGCGGCAG | 25631 |
| 4718 | CGTCCTGTCGTTCGGCGGCA | 25630 |
| 4719 | ACGTCCTGTCGTTCGGCGGC | 25629 |
| 4720 | AACGTCCTGTCGTTCGGCGG | 25628 |
| 4721 | CAACGTCCTGTCGTTCGGCG | 25627 |
| 4722 | CCAACGTCCTGTCGTTCGGC | 25626 |
| 4723 | CCCAACGTCCTGTCGTTCGG | 25625 |
| 4724 | CCCCAACGTCCTGTCGTTCG | 25624 |
| 4725 | GCCCCAACGTCCTGTCGTTC | 25623 |
| 4726 | CGCCCCAACGTCCTGTCGTT | 25622 |
| 4727 | CCGCCCCAACGTCCTGTCGT | 25621 |
| 4728 | GCCGCCCCAACGTCCTGTCG | 25620 |
| 4729 | GGCCGCCCCAACGTCCTGTC | 25619 |
| 4730 | AGGCCGCCCCAACGTCCTGT | 25618 |
| 4731 | CAGGCCGCCCCAACGTCCTG | 25617 |
| 4732 | CCAGGCCGCCCCAACGTCCT | 25616 |
| 4733 | GCCAGGCCGCCCCAACGTCC | 25615 |
| 4734 | AGCCAGGCCGCCCCAACGTC | 25614 |
| 4735 | GAGCCAGGCCGCCCCAACGT | 25613 |
| 4736 | GGAGCCAGGCCGCCCCAACG | 25612 |
| 4737 | GGGAGCCAGGCCGCCCCAAC | 25611 |
| 4738 | AGGGAGCCAGGCCGCCCCAA | 25610 |
| 4739 | GAGGGAGCCAGGCCGCCCCA | 25609 |
| 4740 | TGAGGGAGCCAGGCCGCCCC | 25608 |
| 4741 | CTGAGGGAGCCAGGCCGCCC | 25607 |
| 4742 | CCTGAGGGAGCCAGGCCGCC | 25606 |
| 4743 | ACCTGAGGGAGCCAGGCCGC | 25605 |
| 4744 | TACCTGAGGGAGCCAGGCCG | 25604 |
| 4745 | CGGGAGGCGGTCACATTCGGCGCG | 25690 |
| 4746 | GGGAGGCGGTCACATTCGGC | 25691 |
| 4747 | GGAGGCGGTCACATTCGGCG | 25692 |
| 4748 | GAGGCGGTCACATTCGGCGC | 25693 |
| 4749 | AGGCGGTCACATTCGGCGCG | 25694 |
| 4750 | GGCGGTCACATTCGGCGCGT | 25695 |
| 4751 | GCGGTCACATTCGGCGCGTC | 25696 |
| 4752 | CGGTCACATTCGGCGCGTCC | 25697 |
| 4753 | GGTCACATTCGGCGCGTCCC | 25698 |
| 4754 | GTCACATTCGGCGCGTCCCC | 25699 |
| 4755 | TCACATTCGGCGCGTCCCCA | 25700 |
| 4756 | CACATTCGGCGCGTCCCCAG | 25701 |
| 4757 | ACATTCGGCGCGTCCCCAGC | 25702 |
| 4758 | CATTCGGCGCGTCCCCAGCC | 25703 |
| 4759 | ATTCGGCGCGTCCCCAGCCC | 25704 |
| 4760 | TTCGGCGCGTCCCCAGCCCA | 25705 |
| 4761 | TCGGCGCGTCCCCAGCCCAG | 25706 |
| 4762 | CGGCGCGTCCCCAGCCCAGG | 25707 |
| 4763 | GGCGCGTCCCCAGCCCAGGG | 25708 |
| 4764 | GCGCGTCCCCAGCCCAGGGG | 25709 |
| 4765 | CGCGTCCCCAGCCCAGGGGA | 25710 |
| 4766 | GCGTCCCCAGCCCAGGGGAC | 25711 |
| 4767 | CGTCCCCAGCCCAGGGGACG | 25712 |
| 4768 | GTCCCCAGCCCAGGGGACGG | 25713 |
| 4769 | TCCCCAGCCCAGGGGACGGA | 25714 |
| 4770 | CCCCAGCCCAGGGGACGGAG | 25715 |
| 4771 | CCCAGCCCAGGGGACGGAGC | 25716 |
| 4772 | CCAGCCCAGGGGACGGAGCC | 25717 |
| 4773 | CAGCCCAGGGGACGGAGCCC | 25718 |
| 4774 | AGCCCAGGGGACGGAGCCCC | 25719 |
| 4775 | GCCCAGGGGACGGAGCCCCG | 25720 |
| 4776 | CCCAGGGGACGGAGCCCCGA | 25721 |
| 4777 | CCAGGGGACGGAGCCCCGAG | 25722 |
| 4778 | CAGGGGACGGAGCCCCGAGC | 25723 |
| 4779 | GCGGGAGGCGGTCACATTCG | 25689 |
| 4780 | AGCGGGAGGCGGTCACATTC | 25688 |
| 4781 | GAGCGGGAGGCGGTCACATT | 25687 |
| 4782 | CGGAGCCCCGAGCAGCCCCCGCATCG | 25730 |
| 4783 | GGAGCCCCGAGCAGCCCCCG | 25731 |
| 4784 | GAGCCCCGAGCAGCCCCCGC | 25732 |
| 4785 | AGCCCCGAGCAGCCCCCGCA | 25733 |
| 4786 | GCCCCGAGCAGCCCCCGCAT | 25734 |
| 4787 | CCCCGAGCAGCCCCCGCATC | 25735 |
| 4788 | CCCGAGCAGCCCCCGCATCG | 25736 |
| 4789 | CCGAGCAGCCCCCGCATCGT | 25737 |
| 4790 | CGAGCAGCCCCCGCATCGTA | 25738 |
| 4791 | GAGCAGCCCCCGCATCGTAG | 25739 |
| 4792 | AGCAGCCCCCGCATCGTAGC | 25740 |
| 4793 | GCAGCCCCCGCATCGTAGCA | 25741 |
| 4794 | CAGCCCCCGCATCGTAGCAA | 25742 |
| 4795 | AGCCCCCGCATCGTAGCAAA | 25743 |
| 4796 | GCCCCCGCATCGTAGCAAAC | 25744 |
| 4797 | CCCCCGCATCGTAGCAAACG | 25745 |
| 4798 | CCCCGCATCGTAGCAAACGC | 25746 |
| 4799 | CCCGCATCGTAGCAAACGCG | 25747 |
| 4800 | CCGCATCGTAGCAAACGCGC | 25748 |
| 4801 | CGCATCGTAGCAAACGCGCT | 25749 |
| 4802 | GCATCGTAGCAAACGCGCTC | 25750 |
| 4803 | CATCGTAGCAAACGCGCTCC | 25751 |
| 4804 | ATCGTAGCAAACGCGCTCCG | 25752 |
| 4805 | TCGTAGCAAACGCGCTCCGC | 25753 |
| 4806 | CGTAGCAAACGCGCTCCGCG | 25754 |
| 4807 | GTAGCAAACGCGCTCCGCGC | 25755 |
| 4808 | TAGCAAACGCGCTCCGCGCC | 25756 |
| 4809 | AGCAAACGCGCTCCGCGCCT | 25757 |
| 4810 | GCAAACGCGCTCCGCGCCTC | 25758 |
| 4811 | CAAACGCGCTCCGCGCCTCA | 25759 |
| 4812 | AAACGCGCTCCGCGCCTCAG | 25760 |
| 4813 | AACGCGCTCCGCGCCTCAGG | 25761 |
| 4814 | ACGCGCTCCGCGCCTCAGGG | 25762 |
| 4815 | CGCGCTCCGCGCCTCAGGGC | 25763 |
| 4816 | GCGCTCCGCGCCTCAGGGCA | 25764 |
| 4817 | CGCTCCGCGCCTCAGGGCAC | 25765 |
| 4818 | GCTCCGCGCCTCAGGGCACG | 25766 |
| 4819 | CTCCGCGCCTCAGGGCACGC | 25767 |
| 4820 | TCCGCGCCTCAGGGCACGCG | 25768 |
| 4821 | CCGCGCCTCAGGGCACGCGC | 25769 |
| 4822 | CGCGCCTCAGGGCACGCGCC | 25770 |
| 4823 | GCGCCTCAGGGCACGCGCCC | 25771 |
| 4824 | CGCCTCAGGGCACGCGCCCC | 25772 |
| 4825 | GCCTCAGGGCACGCGCCCCA | 25773 |
| 4826 | CCTCAGGGCACGCGCCCCAA | 25774 |
| 4827 | CTCAGGGCACGCGCCCCAAA | 25775 |
| 4828 | TCAGGGCACGCGCCCCAAAG | 25776 |
| 4829 | CAGGGCACGCGCCCCAAAGC | 25777 |
| 4830 | AGGGCACGCGCCCCAAAGCC | 25778 |
| 4831 | GGGCACGCGCCCCAAAGCCC | 25779 |
| 4832 | GGCACGCGCCCCAAAGCCCG | 25780 |
| 4833 | GCACGCGCCCCAAAGCCCGG | 25781 |
| 4834 | CACGCGCCCCAAAGCCCGGC | 25782 |
| 4835 | ACGCGCCCCAAAGCCCGGCC | 25783 |
| 4836 | CGCGCCCCAAAGCCCGGCCA | 25784 |
| 4837 | GCGCCCCAAAGCCCGGCCAG | 25785 |
| 4838 | CGCCCCAAAGCCCGGCCAGC | 25786 |
| 4839 | GCCCCAAAGCCCGGCCAGCT | 25787 |
| 4840 | CCCCAAAGCCCGGCCAGCTG | 25788 |
| 4841 | CCCAAAGCCCGGCCAGCTGA | 25789 |
| 4842 | CCAAAGCCCGGCCAGCTGAC | 25790 |
| 4843 | CAAAGCCCGGCCAGCTGACC | 25791 |
| 4844 | AAAGCCCGGCCAGCTGACCC | 25792 |
| 4845 | AAGCCCGGCCAGCTGACCCT | 25793 |
| 4846 | AGCCCGGCCAGCTGACCCTT | 25794 |
| 4847 | GCCCGGCCAGCTGACCCTTT | 25795 |
| 4848 | CCCGGCCAGCTGACCCTTTT | 25796 |
| 4849 | CCGGCCAGCTGACCCTTTTC | 25797 |
| 4850 | CGGCCAGCTGACCCTTTTCG | 25798 |
| 4851 | GGCCAGCTGACCCTTTTCGG | 25799 |
| 4852 | GCCAGCTGACCCTTTTCGGG | 25800 |
| 4853 | CCAGCTGACCCTTTTCGGGG | 25801 |
| 4854 | CAGCTGACCCTTTTCGGGGC | 25802 |
| 4855 | AGCTGACCCTTTTCGGGGCC | 25803 |
| 4856 | GCTGACCCTTTTCGGGGCCC | 25804 |
| 4857 | CTGACCCTTTTCGGGGCCCA | 25805 |
| 4858 | TGACCCTTTTCGGGGCCCAA | 25806 |
| 4859 | GACCCTTTTCGGGGCCCAAA | 25807 |
| 4860 | ACCCTTTTCGGGGCCCAAAA | 25808 |
| 4861 | CCCTTTTCGGGGCCCAAAAA | 25809 |
| 4862 | CCTTTTCGGGGCCCAAAAAA | 25810 |
| 4863 | CTTTTCGGGGCCCAAAAAAG | 25811 |
| 4864 | TTTTCGGGGCCCAAAAAAGG | 25812 |
| 4865 | TTTCGGGGCCCAAAAAAGGC | 25813 |
| 4866 | TTCGGGGCCCAAAAAAGGCA | 25814 |
| 4867 | ACGGAGCCCCGAGCAGCCCC | 25729 |
| 4868 | GACGGAGCCCCGAGCAGCCC | 25728 |
| 4869 | GGACGGAGCCCCGAGCAGCC | 25727 |
| 4870 | GGGACGGAGCCCCGAGCAGC | 25726 |
| 4871 | CGCGCTCCGCGCCTCAGGGCACGCGCC | 25763 |
| 4872 | GCGCTCCGCGCCTCAGGGCA | 25764 |
| 4873 | CGCTCCGCGCCTCAGGGCAC | 25765 |
| 4874 | GCTCCGCGCCTCAGGGCACG | 25766 |
| 4875 | CTCCGCGCCTCAGGGCACGC | 25767 |
| 4876 | TCCGCGCCTCAGGGCACGCG | 25768 |
| 4877 | CCGCGCCTCAGGGCACGCGC | 25769 |
| 4878 | CGCGCCTCAGGGCACGCGCC | 25770 |
| 4879 | GCGCCTCAGGGCACGCGCCC | 25771 |
| 4880 | CGCCTCAGGGCACGCGCCCC | 25772 |
| 4881 | GCCTCAGGGCACGCGCCCCA | 25773 |
| 4882 | CCTCAGGGCACGCGCCCCAA | 25774 |
| 4883 | CTCAGGGCACGCGCCCCAAA | 25775 |
| 4884 | TCAGGGCACGCGCCCCAAAG | 25776 |
| 4885 | CAGGGCACGCGCCCCAAAGC | 25777 |
| 4886 | AGGGCACGCGCCCCAAAGCC | 25778 |
| 4887 | GGGCACGCGCCCCAAAGCCC | 25779 |
| 4888 | GGCACGCGCCCCAAAGCCCG | 25780 |
| 4889 | GCACGCGCCCCAAAGCCCGG | 25781 |
| 4890 | CACGCGCCCCAAAGCCCGGC | 25782 |
| 4891 | ACGCGCCCCAAAGCCCGGCC | 25783 |
| 4892 | CGCGCCCCAAAGCCCGGCCA | 25784 |
| 4893 | GCGCCCCAAAGCCCGGCCAG | 25785 |
| 4894 | CGCCCCAAAGCCCGGCCAGC | 25786 |
| 4895 | GCCCCAAAGCCCGGCCAGCT | 25787 |
| 4896 | CCCCAAAGCCCGGCCAGCTG | 25788 |
| 4897 | CCCAAAGCCCGGCCAGCTGA | 25789 |
| 4898 | CCAAAGCCCGGCCAGCTGAC | 25790 |
| 4899 | CAAAGCCCGGCCAGCTGACC | 25791 |
| 4900 | AAAGCCCGGCCAGCTGACCC | 25792 |
| 4901 | AAGCCCGGCCAGCTGACCCT | 25793 |
| 4902 | AGCCCGGCCAGCTGACCCTT | 25794 |
| 4903 | GCCCGGCCAGCTGACCCTTT | 25795 |
| 4904 | CCCGGCCAGCTGACCCTTTT | 25796 |
| 4905 | CCGGCCAGCTGACCCTTTTC | 25797 |
| 4906 | CGGCCAGCTGACCCTTTTCG | 25798 |
| 4907 | GGCCAGCTGACCCTTTTCGG | 25799 |
| 4908 | GCCAGCTGACCCTTTTCGGG | 25800 |
| 4909 | CCAGCTGACCCTTTTCGGGG | 25801 |
| 4910 | CAGCTGACCCTTTTCGGGGC | 25802 |
| 4911 | AGCTGACCCTTTTCGGGGCC | 25803 |
| 4912 | GCTGACCCTTTTCGGGGCCC | 25804 |
| 4913 | CTGACCCTTTTCGGGGCCCA | 25805 |
| 4914 | TGACCCTTTTCGGGGCCCAA | 25806 |
| 4915 | GACCCTTTTCGGGGCCCAAA | 25807 |
| 4916 | ACCCTTTTCGGGGCCCAAAA | 25808 |
| 4917 | CCCTTTTCGGGGCCCAAAAA | 25809 |
| 4918 | CCTTTTCGGGGCCCAAAAAA | 25810 |
| 4919 | CTTTTCGGGGCCCAAAAAAG | 25811 |
| 4920 | TTTTCGGGGCCCAAAAAAGG | 25812 |
| 4921 | TTTCGGGGCCCAAAAAAGGC | 25813 |
| 4922 | TTCGGGGCCCAAAAAAGGCA | 25814 |
| 4923 | ACGCGCTCCGCGCCTCAGGG | 25762 |
| 4924 | AACGCGCTCCGCGCCTCAGG | 25761 |
| 4925 | AAACGCGCTCCGCGCCTCAG | 25760 |
| 4926 | CAAACGCGCTCCGCGCCTCA | 25759 |
| 4927 | GCAAACGCGCTCCGCGCCTC | 25758 |
| 4928 | AGCAAACGCGCTCCGCGCCT | 25757 |
| 4929 | TAGCAAACGCGCTCCGCGCC | 25756 |
| 4930 | GTAGCAAACGCGCTCCGCGC | 25755 |
| 4931 | CGTAGCAAACGCGCTCCGCG | 25754 |
| 4932 | TCGTAGCAAACGCGCTCCGC | 25753 |
| 4933 | ATCGTAGCAAACGCGCTCCG | 25752 |
| 4934 | CATCGTAGCAAACGCGCTCC | 25751 |
| 4935 | GCATCGTAGCAAACGCGCTC | 25750 |
| 4936 | CGCATCGTAGCAAACGCGCT | 25749 |
| 4937 | CCGCATCGTAGCAAACGCGC | 25748 |
| 4938 | CCCGCATCGTAGCAAACGCG | 25747 |
| 4939 | CCCCGCATCGTAGCAAACGC | 25746 |
| 4940 | CCCCCGCATCGTAGCAAACG | 25745 |
| 4941 | GCCCCCGCATCGTAGCAAAC | 25744 |
| 4942 | AGCCCCCGCATCGTAGCAAA | 25743 |
| 4943 | CAGCCCCCGCATCGTAGCAA | 25742 |
| 4944 | GCAGCCCCCGCATCGTAGCA | 25741 |
| 4945 | AGCAGCCCCCGCATCGTAGC | 25740 |
| 4946 | GAGCAGCCCCCGCATCGTAG | 25739 |
| 4947 | CGAGCAGCCCCCGCATCGTA | 25738 |
| 4948 | CCGAGCAGCCCCCGCATCGT | 25737 |
| 4949 | CCCGAGCAGCCCCCGCATCG | 25736 |
| 4950 | CCCCGAGCAGCCCCCGCATC | 25735 |
| 4951 | GCCCCGAGCAGCCCCCGCAT | 25734 |
| 4952 | AGCCCCGAGCAGCCCCCGCA | 25733 |
| 4953 | GAGCCCCGAGCAGCCCCCGC | 25732 |
| 4954 | GGAGCCCCGAGCAGCCCCCG | 25731 |
| 4955 | CGGAGCCCCGAGCAGCCCCC | 25730 |
| 4956 | ACGGAGCCCCGAGCAGCCCC | 25729 |
| 4957 | GACGGAGCCCCGAGCAGCCC | 25728 |
| 4958 | GGACGGAGCCCCGAGCAGCC | 25727 |
| 4959 | GGGACGGAGCCCCGAGCAGC | 25726 |
| 4960 | AGCCGTTCCCGCCTCACAATCG | 25840 |
| 4961 | GCCGTTCCCGCCTCACAATC | 25841 |
| 4962 | CCGTTCCCGCCTCACAATCG | 25842 |
| 4963 | CGTTCCCGCCTCACAATCGT | 25843 |
| 4964 | GTTCCCGCCTCACAATCGTT | 25844 |
| 4965 | TTCCCGCCTCACAATCGTTT | 25845 |
| 4966 | TCCCGCCTCACAATCGTTTT | 25846 |
| 4967 | CCCGCCTCACAATCGTTTTC | 25847 |
| 4968 | CCGCCTCACAATCGTTTTCC | 25848 |
| 4969 | CGCCTCACAATCGTTTTCCT | 25849 |
| 4970 | GCCTCACAATCGTTTTCCTC | 25850 |
| 4971 | CCTCACAATCGTTTTCCTCT | 25851 |
| 4972 | AAGCCGTTCCCGCCTCACAA | 25839 |
| 4973 | AAAGCCGTTCCCGCCTCACA | 25838 |
| 4974 | GAAAGCCGTTCCCGCCTCAC | 25837 |
| 4975 | AGAAAGCCGTTCCCGCCTCA | 25836 |
| 4976 | CAGAAAGCCGTTCCCGCCTC | 25835 |
| 4977 | GCAGAAAGCCGTTCCCGCCT | 25834 |
| 4978 | AGCAGAAAGCCGTTCCCGCC | 25833 |
| 4979 | CAGCAGAAAGCCGTTCCCGC | 25832 |
| 4980 | GCAGCAGAAAGCCGTTCCCG | 25831 |
| 4981 | GGCAGCAGAAAGCCGTTCCC | 25830 |
| 4982 | AGGCAGCAGAAAGCCGTTCC | 25829 |
| 4983 | AAGGCAGCAGAAAGCCGTTC | 25828 |
| 4984 | CCGCCATCTAAGATGGCGGCC | 25876 |
| 4985 | CGCCATCTAAGATGGCGGCC | 25877 |
| 4986 | GCCATCTAAGATGGCGGCCC | 25878 |
| 4987 | CCATCTAAGATGGCGGCCCA | 25879 |
| 4988 | CATCTAAGATGGCGGCCCAA | 25880 |
| 4989 | ATCTAAGATGGCGGCCCAAG | 25881 |
| 4990 | TCTAAGATGGCGGCCCAAGC | 25882 |
| 4991 | CTAAGATGGCGGCCCAAGCG | 25883 |
| 4992 | TAAGATGGCGGCCCAAGCGC | 25884 |
| 4993 | AAGATGGCGGCCCAAGCGCC | 25885 |
| 4994 | AGATGGCGGCCCAAGCGCCC | 25886 |
| 4995 | GATGGCGGCCCAAGCGCCCG | 25887 |
| 4996 | ATGGCGGCCCAAGCGCCCGC | 25888 |
| 4997 | TGGCGGCCCAAGCGCCCGCG | 25889 |
| 4998 | GGCGGCCCAAGCGCCCGCGA | 25890 |
| 4999 | GCGGCCCAAGCGCCCGCGAT | 25891 |
| 5000 | CGGCCCAAGCGCCCGCGATT | 25892 |
| 5001 | GGCCCAAGCGCCCGCGATTA | 25893 |
| 5002 | GCCCAAGCGCCCGCGATTAA | 25894 |
| 5003 | CCCAAGCGCCCGCGATTAAG | 25895 |
| 5004 | CCAAGCGCCCGCGATTAAGA | 25896 |
| 5005 | CAAGCGCCCGCGATTAAGAC | 25897 |
| 5006 | AAGCGCCCGCGATTAAGACT | 25898 |
| 5007 | AGCGCCCGCGATTAAGACTC | 25899 |
| 5008 | GCGCCCGCGATTAAGACTCT | 25900 |
| 5009 | CGCCCGCGATTAAGACTCTC | 25901 |
| 5010 | GCCCGCGATTAAGACTCTCG | 25902 |
| 5011 | CCCGCGATTAAGACTCTCGG | 25903 |
| 5012 | CCGCGATTAAGACTCTCGGG | 25904 |
| 5013 | CGCGATTAAGACTCTCGGGC | 25905 |
| 5014 | GCGATTAAGACTCTCGGGCG | 25906 |
| 5015 | CGATTAAGACTCTCGGGCGG | 25907 |
| 5016 | GATTAAGACTCTCGGGCGGC | 25908 |
| 5017 | ATTAAGACTCTCGGGCGGCC | 25909 |
| 5018 | TTAAGACTCTCGGGCGGCCC | 25910 |
| 5019 | TAAGACTCTCGGGCGGCCCA | 25911 |
| 5020 | AAGACTCTCGGGCGGCCCAG | 25912 |
| 5021 | AGACTCTCGGGCGGCCCAGA | 25913 |
| 5022 | GACTCTCGGGCGGCCCAGAC | 25914 |
| 5023 | ACTCTCGGGCGGCCCAGACG | 25915 |
| 5024 | CTCTCGGGCGGCCCAGACGA | 25916 |
| 5025 | TCTCGGGCGGCCCAGACGAG | 25917 |
| 5026 | CTCGGGCGGCCCAGACGAGC | 25918 |
| 5027 | TCGGGCGGCCCAGACGAGCG | 25919 |
| 5028 | CGGGCGGCCCAGACGAGCGA | 25920 |
| 5029 | CCCGCCATCTAAGATGGCGG | 25875 |
| 5030 | TCCCGCCATCTAAGATGGCG | 25874 |
| 5031 | CTCCCGCCATCTAAGATGGC | 25873 |
| 5032 | ACTCCCGCCATCTAAGATGG | 25872 |
| 5033 | TACTCCCGCCATCTAAGATG | 25871 |
| 5034 | TTACTCCCGCCATCTAAGAT | 25870 |
| 5035 | CTTACTCCCGCCATCTAAGA | 25869 |
| 5036 | TCTTACTCCCGCCATCTAAG | 25868 |
| 5037 | CTCTTACTCCCGCCATCTAA | 25867 |
| 5038 | CCTCTTACTCCCGCCATCTA | 25866 |
| 5039 | TCCTCTTACTCCCGCCATCT | 25865 |
| 5040 | TTCCTCTTACTCCCGCCATC | 25864 |
| 5041 | TTTCCTCTTACTCCCGCCAT | 25863 |
| 5042 | TTTTCCTCTTACTCCCGCCA | 25862 |
| 5043 | GTTTTCCTCTTACTCCCGCC | 25861 |
| 5044 | CGTTTTCCTCTTACTCCCGC | 25860 |
| 5045 | TCGTTTTCCTCTTACTCCCG | 25859 |
| 5046 | ATCGTTTTCCTCTTACTCCC | 25858 |
| 5047 | CGGGCGGCCCAGACGAGCGAGCCCTCG | 25920 |
| 5048 | TCGGGCGGCCCAGACGAGCG | 25919 |
| 5049 | CTCGGGCGGCCCAGACGAGC | 25918 |
| 5050 | TCTCGGGCGGCCCAGACGAG | 25917 |
| 5051 | CTCTCGGGCGGCCCAGACGA | 25916 |
| 5052 | ACTCTCGGGCGGCCCAGACG | 25915 |
| 5053 | GACTCTCGGGCGGCCCAGAC | 25914 |
| 5054 | AGACTCTCGGGCGGCCCAGA | 25913 |
| 5055 | AAGACTCTCGGGCGGCCCAG | 25912 |
| 5056 | TAAGACTCTCGGGCGGCCCA | 25911 |
| 5057 | TTAAGACTCTCGGGCGGCCC | 25910 |
| 5058 | ATTAAGACTCTCGGGCGGCC | 25909 |
| 5059 | GATTAAGACTCTCGGGCGGC | 25908 |
| 5060 | CGATTAAGACTCTCGGGCGG | 25907 |
| 5061 | GCGATTAAGACTCTCGGGCG | 25906 |
| 5062 | CGCGATTAAGACTCTCGGGC | 25905 |
| 5063 | CCGCGATTAAGACTCTCGGG | 25904 |
| 5064 | CCCGCGATTAAGACTCTCGG | 25903 |
| 5065 | GCCCGCGATTAAGACTCTCG | 25902 |
| 5066 | CGCCCGCGATTAAGACTCTC | 25901 |
| 5067 | GCGCCCGCGATTAAGACTCT | 25900 |
| 5068 | AGCGCCCGCGATTAAGACTC | 25899 |
| 5069 | AAGCGCCCGCGATTAAGACT | 25898 |
| 5070 | CAAGCGCCCGCGATTAAGAC | 25897 |
| 5071 | CCAAGCGCCCGCGATTAAGA | 25896 |
| 5072 | CCCAAGCGCCCGCGATTAAG | 25895 |
| 5073 | GCCCAAGCGCCCGCGATTAA | 25894 |
| 5074 | GGCCCAAGCGCCCGCGATTA | 25893 |
| 5075 | CGGCCCAAGCGCCCGCGATT | 25892 |
| 5076 | GCGGCCCAAGCGCCCGCGAT | 25891 |
| 5077 | GGCGGCCCAAGCGCCCGCGA | 25890 |
| 5078 | TGGCGGCCCAAGCGCCCGCG | 25889 |
| 5079 | ATGGCGGCCCAAGCGCCCGC | 25888 |
| 5080 | GATGGCGGCCCAAGCGCCCG | 25887 |
| 5081 | AGATGGCGGCCCAAGCGCCC | 25886 |
| 5082 | AAGATGGCGGCCCAAGCGCC | 25885 |
| 5083 | TAAGATGGCGGCCCAAGCGC | 25884 |
| 5084 | CTAAGATGGCGGCCCAAGCG | 25883 |
| 5085 | TCTAAGATGGCGGCCCAAGC | 25882 |
| 5086 | ATCTAAGATGGCGGCCCAAG | 25881 |
| 5087 | CATCTAAGATGGCGGCCCAA | 25880 |
| 5088 | CCATCTAAGATGGCGGCCCA | 25879 |
| 5089 | GCCATCTAAGATGGCGGCCC | 25878 |
| 5090 | CGCCATCTAAGATGGCGGCC | 25877 |
| 5091 | CCGCCATCTAAGATGGCGGC | 25876 |
| 5092 | CCCGCCATCTAAGATGGCGG | 25875 |
| 5093 | TCCCGCCATCTAAGATGGCG | 25874 |
| 5094 | CTCCCGCCATCTAAGATGGC | 25873 |
| 5095 | ACTCCCGCCATCTAAGATGG | 25872 |
| 5096 | TACTCCCGCCATCTAAGATG | 25871 |
| 5097 | TTACTCCCGCCATCTAAGAT | 25870 |
| 5098 | CTTACTCCCGCCATCTAAGA | 25869 |
| 5099 | TCTTACTCCCGCCATCTAAG | 25868 |
| 5100 | CTCTTACTCCCGCCATCTAA | 25867 |
| 5101 | CCTCTTACTCCCGCCATCTA | 25866 |
| 5102 | TCCTCTTACTCCCGCCATCT | 25865 |
| 5103 | TTCCTCTTACTCCCGCCATC | 25864 |
| 5104 | TTTCCTCTTACTCCCGCCAT | 25863 |
| 5105 | TTTTCCTCTTACTCCCGCCA | 25862 |
| 5106 | GTTTTCCTCTTACTCCCGCC | 25861 |
| 5107 | CGTTTTCCTCTTACTCCCGC | 25860 |
| 5108 | TCGTTTTCCTCTTACTCCCG | 25859 |
| 5109 | ATCGTTTTCCTCTTACTCCC | 25858 |
| 5110 | CGTCCTCCCGACCTGCGACGCCACCGGC | 25957 |
| 5111 | GTCCTCCCGACCTGCGACGC | 25958 |
| 5112 | TCCTCCCGACCTGCGACGCC | 25959 |
| 5113 | CCTCCCGACCTGCGACGCCA | 25960 |
| 5114 | CTCCCGACCTGCGACGCCAC | 25961 |
| 5115 | TCCCGACCTGCGACGCCACC | 25962 |
| 5116 | CCCGACCTGCGACGCCACCG | 25963 |
| 5117 | CCGACCTGCGACGCCACCGG | 25964 |
| 5118 | CGACCTGCGACGCCACCGGC | 25965 |
| 5119 | GACCTGCGACGCCACCGGCT | 25966 |
| 5120 | ACCTGCGACGCCACCGGCTC | 25967 |
| 5121 | CCTGCGACGCCACCGGCTCT | 25968 |
| 5122 | CTGCGACGCCACCGGCTCTC | 25969 |
| 5123 | TGCGACGCCACCGGCTCTCC | 25970 |
| 5124 | GCGACGCCACCGGCTCTCCG | 25971 |
| 5125 | CGACGCCACCGGCTCTCCGA | 25972 |
| 5126 | GACGCCACCGGCTCTCCGAT | 25973 |
| 5127 | ACGCCACCGGCTCTCCGATT | 25974 |
| 5128 | CGCCACCGGCTCTCCGATTC | 25975 |
| 5129 | GCCACCGGCTCTCCGATTCT | 25976 |
| 5130 | CCACCGGCTCTCCGATTCTG | 25977 |
| 5131 | CACCGGCTCTCCGATTCTGC | 25978 |
| 5132 | ACCGGCTCTCCGATTCTGCG | 25979 |
| 5133 | CCGGCTCTCCGATTCTGCGC | 25980 |
| 5134 | CGGCTCTCCGATTCTGCGCG | 25981 |
| 5135 | GGCTCTCCGATTCTGCGCGA | 25982 |
| 5136 | GCTCTCCGATTCTGCGCGAG | 25983 |
| 5137 | CTCTCCGATTCTGCGCGAGC | 25984 |
| 5138 | TCTCCGATTCTGCGCGAGCC | 25985 |
| 5139 | CTCCGATTCTGCGCGAGCCC | 25986 |
| 5140 | TCCGATTCTGCGCGAGCCCT | 25987 |
| 5141 | CCGATTCTGCGCGAGCCCTA | 25988 |
| 5142 | CGATTCTGCGCGAGCCCTAC | 25989 |
| 5143 | GATTCTGCGCGAGCCCTACT | 25990 |
| 5144 | ATTCTGCGCGAGCCCTACTG | 25991 |
| 5145 | TTCTGCGCGAGCCCTACTGG | 25992 |
| 5146 | TCTGCGCGAGCCCTACTGGC | 25993 |
| 5147 | CTGCGCGAGCCCTACTGGCA | 25994 |
| 5148 | TGCGCGAGCCCTACTGGCAG | 25995 |
| 5149 | GCGCGAGCCCTACTGGCAGT | 25996 |
| 5150 | CGCGAGCCCTACTGGCAGTC | 25997 |
| 5151 | GCGAGCCCTACTGGCAGTCG | 25998 |
| 5152 | CGAGCCCTACTGGCAGTCGA | 25999 |
| 5153 | GAGCCCTACTGGCAGTCGAC | 26000 |
| 5154 | AGCCCTACTGGCAGTCGACT | 26001 |
| 5155 | GCCCTACTGGCAGTCGACTT | 26002 |
| 5156 | CCCTACTGGCAGTCGACTTC | 26003 |
| 5157 | CCTACTGGCAGTCGACTTCT | 26004 |
| 5158 | CTACTGGCAGTCGACTTCTA | 26005 |
| 5159 | TACTGGCAGTCGACTTCTAA | 26006 |
| 5160 | ACTGGCAGTCGACTTCTAAC | 26007 |
| 5161 | CTGGCAGTCGACTTCTAACT | 26008 |
| 5162 | TGGCAGTCGACTTCTAACTT | 26009 |
| 5163 | GGCAGTCGACTTCTAACTTG | 26010 |
| 5164 | GCAGTCGACTTCTAACTTGG | 26011 |
| 5165 | CAGTCGACTTCTAACTTGGC | 26012 |
| 5166 | AGTCGACTTCTAACTTGGCT | 26013 |
| 5167 | GTCGACTTCTAACTTGGCTC | 26014 |
| 5168 | TCGACTTCTAACTTGGCTCG | 26015 |
| 5169 | CGACTTCTAACTTGGCTCGG | 26016 |
| 5170 | GACTTCTAACTTGGCTCGGG | 26017 |
| 5171 | ACTTCTAACTTGGCTCGGGC | 26018 |
| 5172 | CTTCTAACTTGGCTCGGGCA | 26019 |
| 5173 | TTCTAACTTGGCTCGGGCAT | 26020 |
| 5174 | TCTAACTTGGCTCGGGCATC | 26021 |
| 5175 | CTAACTTGGCTCGGGCATCC | 26022 |
| 5176 | TAACTTGGCTCGGGCATCCA | 26023 |
| 5177 | AACTTGGCTCGGGCATCCAT | 26024 |
| 5178 | ACTTGGCTCGGGCATCCATC | 26025 |
| 5179 | CTTGGCTCGGGCATCCATCG | 26026 |
| 5180 | TTGGCTCGGGCATCCATCGC | 26027 |
| 5181 | TGGCTCGGGCATCCATCGCT | 26028 |
| 5182 | GGCTCGGGCATCCATCGCTC | 26029 |
| 5183 | GCTCGGGCATCCATCGCTCT | 26030 |
| 5184 | CTCGGGCATCCATCGCTCTG | 26031 |
| 5185 | TCGGGCATCCATCGCTCTGG | 26032 |
| 5186 | CGGGCATCCATCGCTCTGGC | 26033 |
| 5187 | GGGCATCCATCGCTCTGGCC | 26034 |
| 5188 | GGCATCCATCGCTCTGGCCT | 26035 |
| 5189 | GCATCCATCGCTCTGGCCTG | 26036 |
| 5190 | CATCCATCGCTCTGGCCTGA | 26037 |
| 5191 | ATCCATCGCTCTGGCCTGAA | 26038 |
| 5192 | TCCATCGCTCTGGCCTGAAC | 26039 |
| 5193 | CCATCGCTCTGGCCTGAACT | 26040 |
| 5194 | CATCGCTCTGGCCTGAACTC | 26041 |
| 5195 | ATCGCTCTGGCCTGAACTCA | 26042 |
| 5196 | TCGCTCTGGCCTGAACTCAG | 26043 |
| 5197 | CGCTCTGGCCTGAACTCAGG | 26044 |
| 5198 | TCGTCCTCCCGACCTGCGAC | 25956 |
| 5199 | CTCGTCCTCCCGACCTGCGA | 25955 |
| 5200 | GCTCGTCCTCCCGACCTGCG | 25954 |
| 5201 | TGCTCGTCCTCCCGACCTGC | 25953 |
| 5202 | GTGCTCGTCCTCCCGACCTG | 25952 |
| 5203 | GGTGCTCGTCCTCCCGACCT | 25951 |
| 5204 | CGGTGCTCGTCCTCCCGACC | 25950 |
| 5205 | TCGGTGCTCGTCCTCCCGAC | 25949 |
| 5206 | CTCGGTGCTCGTCCTCCCGA | 25948 |
| 5207 | ACTCGGTGCTCGTCCTCCCG | 25947 |
| 5208 | GACTCGGTGCTCGTCCTCCC | 25946 |
| 5209 | CGACTCGGTGCTCGTCCTCC | 25945 |
| 5210 | TCGACTCGGTGCTCGTCCTC | 25944 |
| 5211 | CTCGACTCGGTGCTCGTCCT | 25943 |
| 5212 | CCTCGACTCGGTGCTCGTCC | 25942 |
| 5213 | CCCTCGACTCGGTGCTCGTC | 25941 |
| 5214 | GCCCTCGACTCGGTGCTCGT | 25940 |
| 5215 | AGCCCTCGACTCGGTGCTCG | 25939 |
| 5216 | GAGCCCTCGACTCGGTGCTC | 25938 |
| 5217 | CGAGCCCTCGACTCGGTGCT | 25937 |
| 5218 | GCGAGCCCTCGACTCGGTGC | 25936 |
| 5219 | AGCGAGCCCTCGACTCGGTG | 25935 |
| 5220 | GAGCGAGCCCTCGACTCGGT | 25934 |
| 5221 | CGAGCGAGCCCTCGACTCGG | 25933 |
| 5222 | ACGAGCGAGCCCTCGACTCG | 25932 |
| 5223 | GACGAGCGAGCCCTCGACTC | 25931 |
| 5224 | AGACGAGCGAGCCCTCGACT | 25930 |
| 5225 | CAGACGAGCGAGCCCTCGAC | 25929 |
| 5226 | CCAGACGAGCGAGCCCTCGA | 25928 |
| 5227 | CCCAGACGAGCGAGCCCTCG | 25927 |
| 5228 | GCCCAGACGAGCGAGCCCTC | 25926 |
| 5229 | GGCCCAGACGAGCGAGCCCT | 25925 |
| 5230 | CGGCCCAGACGAGCGAGCCC | 25924 |
| 5231 | GCGGCCCAGACGAGCGAGCC | 25923 |
| 5232 | GGCGGCCCAGACGAGCGAGC | 25922 |

Hot Zones (Relative upstream location to gene start site)
25500-27500

### Examples

Genetic Code (5′ Upstream Region)

(SEQ ID NO: 11966)

AATACAGTCTTCCCCCACAGTTGAATATAGAATAAAATCTATTGCAAGCT
GGGTGCAGGGGCACAAGTGTGGCAGGAGTGCTTGAGCCTAGGAGTTCAAG
ACCAGCCTGGGCAACATAGTGAGACCTCATCTCAATTGAAAATATATATC
TATATAAAAAATAAAATTTATTACAGTTCATCTTGCTGGAAAACAAAATA
CTGTTTTTGTAATTAAAATTTTTTTTTTAAATTTAGAAATGGGGTCTTGC
TGTGTTGACCAGGCTGGTCTTGAACTCTTGGCCTCAAGCTGTCCTCCCAT
CTGGGCCTCCCAAAGTGCTGGGATTACAGGTGTGAACAACTGCGCCCGGC
TGACAAAGTATTTTTTAAAGATGTACCACTAAATGGAGATTTGATTCACA
TTTGATAGTTTTTGACAGGTCTTTTCTATTTAAAAACATTACTGTTTTTG
TAGCATTATTCTGGCTTTTCCCTTAATTTAGTAAATATTTGAGTGCCTTT
GTATTCCAGATACTGAGCAAGATTGGCAGGGTTCTGCCCTTATGGAGCAG
AAGGAAGGTAGGGGGACTGACTAAAACTTGAAAACTGTCTAACATAAGTA
CCATGCAGAAAATGAAACAGTATTAATTGGCAGAAGGAGAGCAGGCTATT
TTGGCTAGTGTGGTTAGGGAAAGCCTCTCTAAAGAGATGTCTCTTGGGTG
GAGACAAGATGTGAAAAAACCAGCTTGCCTGTTTTTGGGGTTTCAGCCTT
GCAGGTGAAGAGAAACACGAAGTTCAGAAGTCTTGAGGCACAAAGTCTGG
CATGTTACGAAAGAAGGCCTTTAGACGCCTTGTCAGGGAGTTTAGATTTT
ATTCTGAGTTTTAAAACGGGAGTGACACAATGAGTTGCATTTTAAGCCTG
TTCAGGCTGTTACATGGATTATTAGGAGCTGTATCATTTCAGGCTAGTGA
GATGCTCAGATGAGTCTGCCTTCTGTCTCTTCCGTCATCTATTTCTCTCT
TATCTGGTCTTAAGCTCCTCCATCTTTTCCTTTTTAGTTGGAAAAAAACT
CAAAGATCTAGAAAAAAGAGGAGCTGTATGTACTCCTAAAAAGGGACCTC
ATAGTAACCTGGGGATAGAGTTATGTAGGAGTGAGTCAGGGCTCAGGTTG
AGGCTTTAGAGGCAGGAGGCAGCGAGATCTTGTTCTGTCATCCCCTCTTA
CAGAAATAAAATATGCCGATAAAAGTTTATAGTGTAATAGTAAAATATAA
AAACAAAAAGTAAGTAATGTAGAAAATAAAAACCCTTCACAGTCCTGCTG
AAATGATTACTGTTAACACTTTAATTCTAGAGTTCCCCATCCATTTATTT
ATTTCTAGATTTCCCTCTTTGTAGATTAATATTAAAGGGTTCAGACTTGT
TCATTTTTTGTTGTCTTGGATATCTTTTCCCACCTCTGTATATATGGATC
TACTTTATTTATCACGTGGATATTAACATGGTTTATTTAATTCCCTATTG
TTAGGTATTTGGTCTTTACCACAGTTTTTCAAGGGTATGAATAGTGCTGC
AAGGAATATGCTTACACATGTTTTTATACACTTGTCTTAGGCTTCTGTAG
GACAAATTTCTGGAGTAGAATACTAGGTCATTCTTTAAGAACATTTCAAA
CTTTTAATAGATATTACCGTATTCTTTCCCAAAAAGAATGTACAAAGACT
GTATGAGAATAACTCCATGTTGTGATCTTAAGTTGTCTCTAAACCTCTTT
GGTTTTCTTAGCTGTCATCTAAGAATACTAAGTATCTAACCTCCCTCTTG
ATTTGGGCATGTGATGTGATTTAGCATATAGTGGATATTCAGTTAGAAAC
TTTTGGTTGAAAACAAGGTTTGGATTCTGTGGTCTTTAATTCTAGGCCAT
TTCAGCTCTGACTAAAATGATTTGAGTGTTAGTGTTATATATGGGAAGGT
AAGGGCTATGGAGTCAGTGCAGCCCAGTTCAGAATCCCAGTTTGCCACTT
ACAAGCTGTGTGTGTGAGAATTTTCTCAACTGTAAAATGGGGACATAATT
CCTACCTAGAGTAATACTGTAAGTATTAAGGTGGATAATGATTGGAATGT
ATGCTGTGTATCCTGCCTCATAATAGTAAGCTTTTAGTAAATGGTAGCTA
CTGTTAATAATAAAACAAGTTTCTGAAGGAGGAAGGCTTGAAAAGATGGG
ATTCCTTATCAACCTCAAAGTTTTCTAAAGGAGGAAACCCTACCCCCCTT
ACTTCTGCATGGTTTCTGACCATGAACTGAACTCTGAACTCTGAATGAAC
TGAACTCTGAACTCTGAATGAACTGAACTCTGAACTCTGAATGTTATGGT
AGAAAATTCATGGACTTTAAATTTAAACAGATAAAGAATCTGGTTATTTT
ACCCACTGCTGGGGTGTTCTTGGGCAAGTAGCATGACTTCTGTGTCCAAA
AAAGAAAGGGTTTGCAGTGACTGAACCTGTAATCCCAGTACTTTGGGAGG
CTAAGGAGAGTGGATTGCCTGAGCTCAGGAGTTCAAGACCAGCCTGGGCA
ACATAGTGAGAGCCTTTCTCAACAAAAAAAACTGTTCTTAAAAATTAGCT
GGGCATGGTGATGCACGTCTGTGGTCCCAGCTATGTGGGAAGCTGAGGTA
GGAGAATCATTTGAGCCTGGAAAATTGAAGCTGCAGTGAGCTGTGATCAT
GTCACTGCACCCCAGCCTGGGCAACAGAGCAAGACCCTGTCTCAGAAAAT
AAATTAATTAAAAAGAAAGTGTGGATGGAGGAAGGGATTAAAAATCTGGC
TGGGCACGGTGGCTCATGCCTGTAATCCCAGGCGTGATTTGGGAGGCCGA
GGCGGACAGATCACGAGGTCAAGAGATTGAGACCATCCTGGCCAACATGG
CCAACCCCATCTCTACTAAAAATACAAAAATCAGTCGGGCGTGGTGGTGC
ATGCCTGTAATCCCGGCTACTCGGGAGGCTGAGGCAGGAGAATCGCTTGA
ACCTGGGAGGTTCAGTGAGCCAAGATCGCGCCACTACACTCCAGCCTGGC
AATAGAGTGAGACTCTGTCTCAAAAGAAAAGAAAAGAAAAGAAAATCTTT
GGGGTTCTTACACAAATTAAATGAGATAATTTATTATTATTATTTTTTTT
GAGATGGAGTCTTGCTCTGTCCCCCAGGCTGGAGTGCAGTGGTGCGATCT
CAGCTCACCGCAAGCTCTGCCTCCCGGGTTCACGCCATTCCCCTGCCTCA
GCCTCCTGAGTAGCTGGGACTACAGGCGCCCGCCACCATGCCTGGCTAAT
TTTTTGTATTTTTAGTAGAGACAGGGTATCCCTGTGTTAGCTAGGATGGT
CTCGATCTCCTGACCTTGTGATCCGCCCATCTCGGCCTCCCAAAGTGCTG
GGATTACAGGTATGAGCCACCATGCCCGGCTTGAGATAATTTATAAAGTG
CCTAAAATACATCCTAGAAATATTAGTTTTTCTTCCTTGAAGTCATAAAT
TATGGCTTACACTTTTTTTCAGGTATTTCTCATAGTACTAATGTGTTGCT
CACACTCAAGGGTAGTAGTTGCTTAGGAAGAAGAGAAATGTAGTTGAAAA
AGTAATAGACTAGAAGTCTTGAGACCTGGGCTCATGTTCCAAGTTGGCTT
TTTTTTTTTTTTTTGGGAGATGGAGTCTCGCTCTTGTCCCCCAGCCTGGA
GTGCAATGACACGATATCGACTCACTGCAACCTCCACCTCCTGGGTTCAA
GTGATTTCTCCTGCCTCAGCCTCCCTAGTAGCTGGGATGACAGACACCCA
CCACCATGCCTGGCTAATTTTTGTATTTTAAGTAGTGACAGCATTTTACC
ATGTTAGCCAGGCTGGTCTTGAACTCCTGGCCTCAAGTGATGCGCTGGCC
TCGGCCTCCCAAAGTGCTGGGATTACAGGCATGAGCCACTGTGCCTGGTC
CCTTGCTAAATGTTTTGTTTTGTTTTGTTTTGTTTTTGAGGTGGAGTCTT
GCTCTGTCACCCAGGCTGGAGTGCGGTGGCATGATCTCCGCTCACTGCAA
GCTCCGCCTCCCAGGTTCCCGCCATTCTCCTGCCTCAGCCTCCCGAGTAG
CTGGGACTACAGGCGCCCGCCACCACGCCCGGCTAATTTTTTGTATTTTT
AGTAGAGATGGGGTTTCACCGTGTTAGCCAGGATGGTCTCCATCTCCTGA
CCTCGTGATGCACCCACCTCGGCCTCCCAAAGTGCTGGGATTACAGGCGT
GAGCCACCGTGCCCCGCAGTTGCTTGCTAAATCTTTTAACTGCTGGTCCC
ATTTTCCTCATCTATGAAATATTTAATGGAAGTGTACTATTAAAGAAACT
TTTCTTTGCTGATGAATGCAGGAGGTATCATTAAAAACCCACATAGTGCT
ATTTTCATAATTACTCTTTATGTATTGTGTTCTTGGGTTGAATACTTTTG
TTCTAGAGTTACAATTATTTGTGTTTCTTACCAGGTTTAAGAATTGTTTA
AGCTGCATCAATG

18) Beta catenin. Proto-oncogene protein Wnt-1 is a protein that in humans is encoded by the WNT1 gene (Van Ooyen et. al, 1986; Nat. Genet. 28 (3): 261-5 and Aarheden et al., 1988; Cytogenet Cell Genet 47 (1-2): 86-87). The WNT gene family consists of structurally related genes that encode secreted signaling proteins that are implicated in oncogenesis and in several developmental processes, including regulation of cell fate and patterning during embryogenesis. Wnt-1 t is conserved in evolution with the protein encoded by this gene having 98% identity to the mouse Wnt1 protein at the amino acid level.

Beta-catenin (or β-catenin) is a protein that in humans is encoded by the CTNNB1 gene. β-catenin is a subunit of the cadherin protein complex and acts as an intracellular signal transducer in the Wnt signaling pathway (McDonald et al, 2009; Developmental Cell 17 (1): 9-26). Recent evidence suggests that β-catenin plays an important role in various aspects of liver biology including liver development (both embryonic and postnatal), liver regeneration following partial hepatectomy, HGF-induced hepatomegaly, liver zonation, and pathogenesis of liver cancer (Thompson and Monga, 2007; Hepatology 45 (5): 1298-305). The gene that codes for β-catenin can function as an oncogene. An increase in β-catenin production has been noted in those people with basal cell carcinoma and leads to the increase in proliferation of related tumors (Saldanha et al, 2004; Cancer Epidemiol. Biomarkers Prev. 17 (8): 2101-8. Mutations in this gene are a cause of colorectal cancer (CRC), pilomatrixoma (PTR), medulloblastoma (MDB), and ovarian cancer. Also, β-catenin binds to the product of the APC gene, which is mutated in adenomatous polyposis of the colon (reviewed in Wang et al, 2008; Cancer Epidemiol. Biomarkers Prev. 17 (8): 2101-8).

Protein: Beta-catenin Gene: CTNNB1 (Homo sapiens, chromosome 3, 41240942-41281939 [NCBI Reference Sequence: NC_{—}000003.11]; start site location: 41265560; strand: positive)

Gene Identification

| | |
|---|---|
| GeneID | 1499 |
| HGNC | 2514 |
| HPRD | — |
| MIM | 116806 |

Targeted Sequences

| | | | Relative |
|---|---|---|---|
| | | | upstream |
| | | | location |
| | | | to gene |
| Sequence | Designed | | start |
| ID No: | ID | Sequence (5′-3′) | site |
| 5233 | BC1 | CGCATATTACTGGGTAAACTCTGTG | 1411 |
| 5234 | | CACGCTGGATTTTCAAAACAGTTG | 5 |

Target Shift Sequences

| | | Relative |
|---|---|---|
| | | upstream |
| Sequence | | location to |
| ID No: | Sequence (5′-3′) | gene start site |
| 5233 | CGCATATTACTGGGTAAACTCTGTG | 1411 |
| 5234 | CACGCTGGATTTTCAAAACAGTTG | 5 |
| 11987 | CCACGCTGGATTTTCAAAAC | 4 |

Hot Zones (Relative upstream location to gene start site)
1-250
1400-1500

### Examples

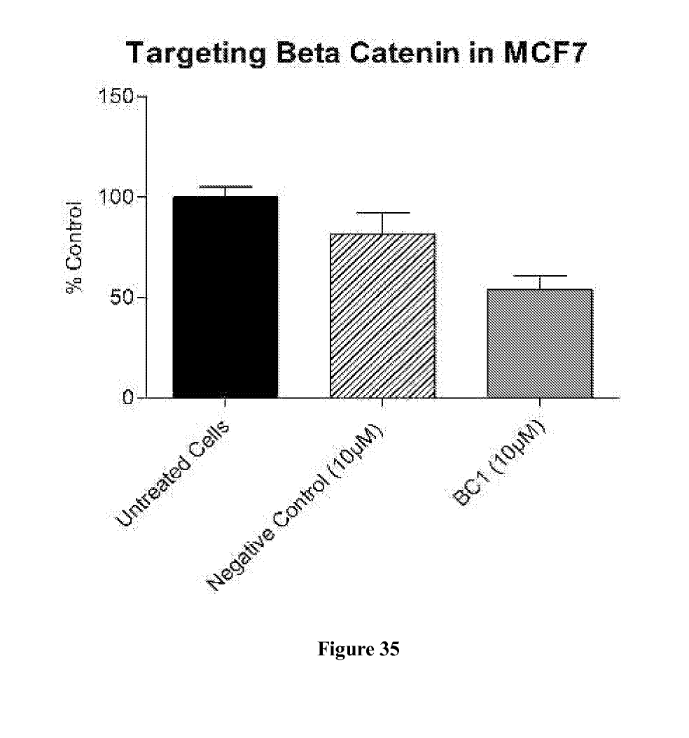

In FIG. 35, In MCF7 (human mammary breast cell line), BC1 (191) produced statistically significant (P<0.05) inhibition at 10 μM compared to the untreated control values. The β-catenin sequence BC1 fits the independent and dependent DNAi motif claims.

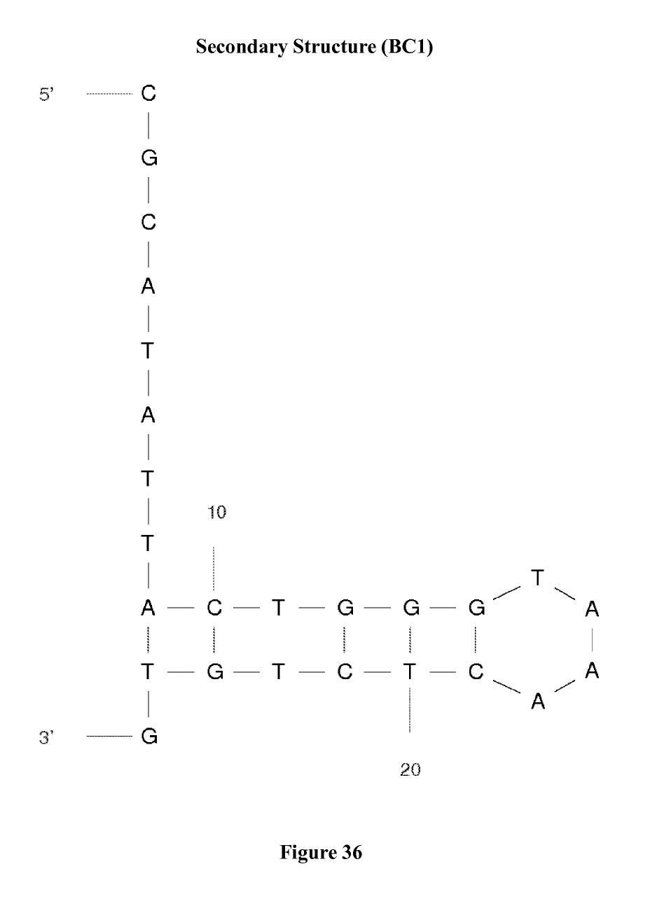

The secondary structure for BC1 (191) is shown in FIG. 36.

Genetic Code (5′ Upstream Region)

(SEQ ID NO: 11967)

ACCCTGTAGGATGGGCGGGTGATGGTATGTATGTTAGATGTGTGGACATA
TCTATTAAAAGTTGTGTCAGATAACAGCTGGTGCTGACAAGCCCTTGGTA
AGATGGCAGCATGTTCAATATGTTCTGTGAAAATTATCTCAGTTTATGAT
CTGTCAGTATTGTGGAGCTATGCATGAAAGGACTTAAAATTCTTACCCTT
AAACTCAGTAACAGTGTTTCTAGAACTTCTGGTGATATGGGAAATTAAGA
GAATTATTTATATGCAAAGGTGTTTATTGCAGCATTGTTGGAATAATAGA
CAAAATGGGGAAGAACAAGCTCAGAATGGAGGAGGTAGCTTATAGTATAG
ACATACGATACAATCCAGATGATAATATTTTATAATAGTCTTCACAAGGA
ATTTTATATTTTTATTTTTAAAAATACATAGCAGTGAGTTTAATATACCA
AACATACCAAAATGTCATCATTTACTGTGTGGTGGACTCATATGATGGAG
ATGATAAATAAAAATATTAATTTATTTGAGGCATATATTTATGGCTGAGG
AAGGAAGACAGTTATGAAGAACAGCTCATTCTGGAAACATACTAATTTTT
CCCAGCCATAAAGAGATTTCCTATTTCTTTTTTTTTTCCATTTACCTTCT
GTTTCCTACCTGAGAAGATTTCATACTTCTAATAACCATTTGTGTACCTA
TTTAAAGACAGTACCAAAGGCATACATTTTAGTGTTTGGAGGACCAAGGG
TCATTTGATGTTTGATGCTTATTGACTATTCGAGGATGACAAGACACCTT
GAGAACACACACACCCACACCCACACCCACACCCTCACCCACCCACCCCA
CCCCCCTCCCCGAAGAAAGCTGTGAAGGAAGAAAGCAGAAAAGAACCTGG
AGTGAGTTGTAACTTAAAATGTTAGTGTTGCATGAAGTGTGTTAAAACAG
GAAGATTTGAGGAAATTGCATACATTTTCTAGATGGCAAAGTATTACTGG
TGACAGTTAATGAAAATGCATATGCATGTGTTTTTAGATTTACAAATTTT
ACTAAGAACTTTTTAAAAATCCCTGAAGGTGTATCAAAAGTTTATCATGC
TTATGAAATAGAGTAGCACTTTCTAACTTTAAAACGGGGAATAATTCTTT
GGATCTTGATTATTGGAAAAGTGAATTATGAATTGCTAGTATAAAACTGT
GGTTTTAAAATATGTCTGCTTTATATTTTTATGTAGCAGATTTACTCCTA
GTTAATAATACTCAAACTTACTGAAAACTAAGGTAATTAAGATAATTCTG
TCCTGATGGGAAGAGGAAAAATAACTTCAGTGTGAAATCTATTATATATT
AGTTGTGGCAAGATTTCTCCCATTGACTTTGACTGGAGACATTTATAGGG
TTAAAATCGGAAATAGCACGGTGAATTTTGAAGTATCCTTGTAGTTGGAA
AGAGTATTATGTTCATATTGCCAAAAAAAAGATGCATGGATGCATTAGAC
TGGATGGAAAATACATGAGAAGTTGGCTAGCCCCCTCTTTGTCAAAACAT
CACTTGGTGGTGATAAAGCTGTTGGAAAACACAGCATTCTAATGTAGTCT
GTAGTTTAATGATAATCTGTGTCTTGAAACATTTAGCGTAGTACTTATAC
AAACCTAGATGGCATAGTGTACTGCATGCCTAGCCTATATAGTATAGCCT
GTTGCTTCTAGGGTGTAAAGCTGTATAGCGTGTTACTATAGGCAGTTGAA
ACAGTGGTATTTATGTATCCTTTTTTTTTTTTTTAAATTCTTTTAAGAGA
CAGGGTCTTGCTCTGTTGCCCAGGCTGGATGCATTGGTGTGATCATAGCT
CACTATAACCTTGAACTCCTAAGTGATCCTCTTTGCCTCAGCCTCCCCAG
TGGCTAGGACTACAGGCACATACTACCACACCTGGCTAATTTTTAACATT
TTTTTGTAGAGATGGAATTTCGCTGTGTTGTCCAGGCTGGTCTTGGAACT
CTTGTGCTGCAGCAATCCACCCGCCTCCCAAAGTGTTAGAATTACAAGCC
ACTTCGCCTGGCTTGTTTACCTAAACATAGAAAAGATCCAGTAAAAATAC
AGAATTAAAATCTTGTGGGGCCACTGTAGCATATGTAGTCCATCTTGACT
GAAATGTCCTTATGCAGTGCATGATTGTACTTCATAATTTTTAAGCACTC
CTCCCTCTTGATTGGTACTTAGTGGATTTTATCATTTTTGTTTCTTCATA
ATTCTTTCTGAAATGTCTACTGGTTGGACCTTTGATCTCCTGAATTGATC
GTGATTTCTTCTGTTGTATTTTTTGTCTTTGTCATTTTTTTGTACTCTAG
GCAGTTTTCTCAATTTTAGTTTCTATTCAACTTTTTGTTTTTATTTATTC
TCTCCAGTATTTATGGAGATACTAAATTGAAGTGTTCTGTTTCTCTCTCC
ACCCTATCCCTAGTTTCAAGTTTTATCTCAGTTTCTATGGAGTCAGTTTT
TTCGTTGCTTTAAAAAAAAATTTTCCTGAAGTGATTGGTAAGTTTTGGCT
AATTGGGAGCACTAGAATTGGGCCCTTAATGGTTGGCAGGGTGTGGTGGA
GGAGAGACAGCCCTTAGTCCAAAGGCTCAGGCCAGAAAAAGAAAGAGGAA
GGCTTTCCTTTTCCTTTCCGGAGCAGGGTTCTGCCCTAGGTCTTGCTTGG
CAGTCTATTTGATTTCTTTAGCAGTTAATGCTCAGTTTTTTGGCATATGT
GGATCTGCCTCCAGAGCAGGTACAAGGTGAGTGAGTCTATGCTGTTACCT
AATTAGATCCCCATTTCTACCCTTTGTTTTTACTTCTCTATCTACTGATA
GGTTTTTACCCTCCTTCACCTCATAGGGTTGCAGTGAAGAGCAAGATGAA
TTTTTATTTATGTTGCATAAATTTTAAAAGCTAAAAAATATATATGTAAT
GTTGGGAAGTCCCAGTGTACAAATGGCTATTGTAAATTTGGAACATGAAC
TTGCTTTTTTCCATTGTAAAAATGAAATCATTATAAATTGCGGTCAAGTT
ACTAGGTCAGCCCACACAGAGTTTACCCAGTAATATGCGTAAATGTTTTG
CCTTTGCATCAACAACAAGGAAAAACAGTACTATAAAAAAATGTTCCTGG
AAGCCGGATGTATCAAAGCACTTCTGAAATAGCTATATAGCCTATAGACA
TGACCAGTTGGTTTCTGAGTCTGTTGACATTGGCCAAAGGAGAAGCTCAG
TGTAGAACATGTTTGGAGTCTCCTTTTGCAGAAATACATTGGAGGCTGGA
GTGGGGAACCAATTTTTCAGAAAGGTGGTGAAGTAGTTACATAGCCACTC
TTTTAAAGACAGTCAAAAGATAGAAACTAAGGCCAGGTGTTGGCTCACAT
CTGAGATAGGAAAATCACTTGAACCTGGGAGGCGGAGGTTGCAGTGAGCC
CAGTATGCACCTCTGCACTCCAGCCTGGTTTGGCAAGAGACCAAAACTCT
GTCTCAAAAAAAAACAAAACATAGTTCACACTTAAATATTTTATTCCATA
TCTTTACATACCCAATATGTTAATTTATAGTTCAAGATGAACTTGTTTGG
GACAGATTTTGTAATAAAGGAAATCGTGTTATTAGAAATATCTAGAGGCC
ATGAGCCCTTAAACTGTTCTAATTTGCAAGTAGTTCCCTGTGTGATGCAG
TTTTTTTCAATATTGCACAATAAAGGCAAAATACGGACAAATTAGATGAT
AAGATTTATATAAATTTTTAAAATATTGATCAAAATATGTATCCATATTG
GTAATATTTGTATTTATAATAAATCATTGCTGTAAATTTGAACTTAGAAA
AATTTTACTAATAAAGGTGCTTTTGTGTTGCAAACTTTCATTTGAAAAGT
AATTTTTCTTTGTACCAAAAAATCTAAAATTCGCTATTCTAGTCACCAAA
ATTTGCTTTATGAAAAATAATTTTTGATGGCACTATATCAGAAAACAACT
TGTTAAAGAAAATGTGGAGTTTTTAAAATCCCACTGTACCTCTGTTATCC
AAAGGGGATCTGTGAATTTTTCTGTGAAAGGTTAAAAAAGGAGAGACCTT
TAGGAATTCAGAGAGCAGCTGATTTTTGAATAGTGTTTTCCCCTCCCTGG
CTTTTATTATTACAACTCTGTGCTTTTTCATCACCATCCTGAATATCTAT
AATTAATATTTATACTATTAATAAAAAGACATTTTTGGTAAGGAGGAGTT
TTCACTGAAGTTCAGCAGTGATGGAGCTGTGGTTGAGGTGTCTGGAGGAG
ACCATGAGGTCTGCGTTTCACTAACCTGGTAAAAGAGGATATGGGTTTTT
TTTGTGGGTGTAATAGTGACATTTAACAGGTATCCCAGTGACTTAGGAGT
ATTAATCAAGCTAAATTTAAATCCTAATGACTTTTGATTAACTTTTTTTA
GGGTATTTGAAGTATACCATACAACTGTTTTGAAAATCCAGCGTGGACA
ATG

19) PCSK9. Proprotein convertase subtilisin/kexin type 9, also known as PCSK9, is an enzyme that in humans is encoded by the PCSK9 gene. This gene encodes a proprotein convertase belonging to the proteinase K subfamily of the secretory subtilase family. The encoded protein is synthesized as a soluble zymogen that undergoes autocatalytic intramolecular processing in the endoplasmic reticulum. This protein plays a major regulatory role in cholesterol homeostasis. PCSK9 binds to the epidermal growth factor-like repeat A (EGF-A) domain of the low-density lipoprotein receptor (LDLR), inducing LDLR degradation. Reduced LDLR levels result in decreased metabolism of low-density lipoproteins, which could lead to hypercholesterolemia. Variants of PCSK9 can reduce or increase circulating cholesterol. LDL cholesterol is removed from the blood when it binds to LDL receptors on the surface of liver cells, and is taken inside the cells. When PCSK9 binds to the LDL receptor, the receptor is destroyed along with the LDL. But if PCSK9 does not bind, the receptor can return to the surface of the cell and remove more cholesterol (reviewed in Akram et al, 2010 Arterioscler Thromb Vasc Biol.; 30:1279-1281)

There are numerous approaches to inhibiting PCSK9 being developed as a means of lowering cholesterol levels (reviewed in Lambert et al., 2012; J Lipid Res. 53(12):2515-24). A number of monoclonal antibodies that bind to PCSK9 near the catalytic domain that interact with the LDLR and hence inhibit the function of PCSK9 are currently in clinical trials including AMG145, 1D05-IgG2, and SAR236553/REGN727 (Aventis/Regeneron). Peptide mimetics and oligonucleotide approaches are also being developed. These include a mimic of the EGFA domain of the LDLR that binds to PCSK9, an antisense PCSK9 oligonucleotide, a locked nucleic acid inhibitor and siRNA approaches.

Protein: PCSK9 Gene: PCSK9 (Homo sapiens, chromosome 1, 55505149-55530526 [NCBI Reference Sequence: NC_{—}000001.10]; start site location: 55505511; strand: positive)

Gene Identification

| | |
|---|---|
| GeneID | 255738 |
| HGNC | 20001 |
| HPRD | 07080 |
| MIM | 607789 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 5235 | | CAGGGCGCGTGAAGGGGCGCGCGG | 120 |
| 5236 | | GACGCGTCCCGGCCCGCCCGAGC | 179 |
| 5285 | | GACGCCTGGGGCGCGCAGATCAC | 341 |
| 5341 | | CAGGCCGGCGCCCTAGGGGCTCC | 494 |
| 5359 | | CACGCCGGCGGCGCCTTGAGCC | 56 |
| 5402 | PC2 | CAGGTTTCGGCCTCGCCCTCCC | 408 |
| 5445 | | CATCGAGCCCGCCATCGCAGCAC | 1307 |
| 5473 | | GAGCGCCTCGACGTCGCTGCGGAAACC | 273 |

Target Shift Sequence

| | | Relative |
|---|---|---|
| | | upstream |
| | | location |
| Sequence | | to gene |
| ID No: | Sequence (5′-3′) | start site |
| 5235 | CAGGGCGCGTGAAGGGGCGCGCGG | 120 |
| 5236 | GACGCGTCCCGGCCCGCCCGAGC | 179 |
| 5237 | ACGCGTCCCGGCCCGCCCGA | 180 |
| 5238 | CGCGTCCCGGCCCGCCCGAG | 181 |
| 5239 | GCGTCCCGGCCCGCCCGAGC | 182 |
| 5240 | CGTCCCGGCCCGCCCGAGCC | 183 |
| 5241 | GTCCCGGCCCGCCCGAGCCA | 184 |
| 5242 | TCCCGGCCCGCCCGAGCCAG | 185 |
| 5243 | CCCGGCCCGCCCGAGCCAGT | 186 |
| 5244 | CCGGCCCGCCCGAGCCAGTC | 187 |
| 5245 | CGGCCCGCCCGAGCCAGTCT | 188 |
| 5246 | GGCCCGCCCGAGCCAGTCTC | 189 |
| 5247 | GCCCGCCCGAGCCAGTCTCA | 190 |
| 5248 | CCCGCCCGAGCCAGTCTCAC | 191 |
| 5249 | CCGCCCGAGCCAGTCTCACT | 192 |
| 5250 | CGCCCGAGCCAGTCTCACTG | 193 |
| 5251 | GCCCGAGCCAGTCTCACTGC | 194 |
| 5252 | CCCGAGCCAGTCTCACTGCC | 195 |
| 5253 | CCGAGCCAGTCTCACTGCCT | 196 |
| 5254 | CGAGCCAGTCTCACTGCCTG | 197 |
| 5255 | CGACGCGTCCCGGCCCGCCC | 178 |
| 5256 | ACGACGCGTCCCGGCCCGCC | 177 |
| 5257 | AACGACGCGTCCCGGCCCGC | 176 |
| 5258 | CAACGACGCGTCCCGGCCCG | 175 |
| 5259 | GCAACGACGCGTCCCGGCCC | 174 |
| 5260 | TGCAACGACGCGTCCCGGCC | 173 |
| 5261 | CTGCAACGACGCGTCCCGGC | 172 |
| 5262 | GCTGCAACGACGCGTCCCGG | 171 |
| 5263 | TGCTGCAACGACGCGTCCCG | 170 |
| 5264 | CTGCTGCAACGACGCGTCCC | 169 |
| 5265 | GCTGCTGCAACGACGCGTCC | 168 |
| 5266 | CGCTGCTGCAACGACGCGTC | 167 |
| 5267 | CCGCTGCTGCAACGACGCGT | 166 |
| 5268 | GCCGCTGCTGCAACGACGCG | 165 |
| 5269 | AGCCGCTGCTGCAACGACGC | 164 |
| 5270 | GAGCCGCTGCTGCAACGACG | 163 |
| 5271 | GGAGCCGCTGCTGCAACGAC | 162 |
| 5272 | GGGAGCCGCTGCTGCAACGA | 161 |
| 5273 | TGGGAGCCGCTGCTGCAACG | 160 |
| 5274 | CTGGGAGCCGCTGCTGCAAC | 159 |
| 5275 | GCTGGGAGCCGCTGCTGCAA | 158 |
| 5276 | AGCTGGGAGCCGCTGCTGCA | 157 |
| 5277 | GAGCTGGGAGCCGCTGCTGC | 156 |
| 5278 | GGAGCTGGGAGCCGCTGCTG | 155 |
| 5279 | GGGAGCTGGGAGCCGCTGCT | 154 |
| 5280 | TGGGAGCTGGGAGCCGCTGC | 153 |
| 5281 | CTGGGAGCTGGGAGCCGCTG | 152 |
| 5282 | GCTGGGAGCTGGGAGCCGCT | 151 |
| 5283 | GGCTGGGAGCTGGGAGCCGC | 150 |
| 5284 | TGGCTGGGAGCTGGGAGCCG | 149 |
| 5285 | GACGCCTGGGGCGCGCAGATCAC | 319 |
| 5286 | ACGCCTGGGGCGCGCAGATC | 320 |
| 5287 | CGCCTGGGGCGCGCAGATCA | 321 |
| 5288 | GCCTGGGGCGCGCAGATCAC | 322 |
| 5289 | CCTGGGGCGCGCAGATCACG | 323 |
| 5290 | CTGGGGCGCGCAGATCACGC | 324 |
| 5291 | TGGGGCGCGCAGATCACGCC | 325 |
| 5292 | GGGGCGCGCAGATCACGCCA | 326 |
| 5293 | GGGCGCGCAGATCACGCCAC | 327 |
| 5294 | GGCGCGCAGATCACGCCACC | 328 |
| 5295 | GCGCGCAGATCACGCCACCA | 329 |
| 5296 | CGCGCAGATCACGCCACCAG | 330 |
| 5297 | GCGCAGATCACGCCACCAGA | 331 |
| 5298 | CGCAGATCACGCCACCAGAG | 332 |
| 5299 | GCAGATCACGCCACCAGAGC | 333 |
| 5300 | CAGATCACGCCACCAGAGCC | 334 |
| 5301 | AGATCACGCCACCAGAGCCC | 335 |
| 5302 | GATCACGCCACCAGAGCCCC | 336 |
| 5303 | ATCACGCCACCAGAGCCCCA | 337 |
| 5304 | TCACGCCACCAGAGCCCCAT | 338 |
| 5305 | CACGCCACCAGAGCCCCATC | 339 |
| 5306 | ACGCCACCAGAGCCCCATCG | 340 |
| 5307 | CGCCACCAGAGCCCCATCGG | 341 |
| 5308 | GCCACCAGAGCCCCATCGGA | 342 |
| 5309 | CCACCAGAGCCCCATCGGAC | 343 |
| 5310 | CACCAGAGCCCCATCGGACG | 344 |
| 5311 | ACCAGAGCCCCATCGGACGA | 345 |
| 5312 | CCAGAGCCCCATCGGACGAT | 346 |
| 5313 | CAGAGCCCCATCGGACGATC | 347 |
| 5314 | AGAGCCCCATCGGACGATCC | 348 |
| 5315 | GAGCCCCATCGGACGATCCT | 349 |
| 5316 | AGCCCCATCGGACGATCCTA | 350 |
| 5317 | GCCCCATCGGACGATCCTAT | 351 |
| 5318 | CCCCATCGGACGATCCTATC | 352 |
| 5319 | CCCATCGGACGATCCTATCT | 353 |
| 5320 | CCATCGGACGATCCTATCTG | 354 |
| 5321 | CATCGGACGATCCTATCTGA | 355 |
| 5322 | ATCGGACGATCCTATCTGAT | 356 |
| 5323 | TCGGACGATCCTATCTGATT | 357 |
| 5324 | CGGACGATCCTATCTGATTA | 358 |
| 5325 | TGACGCCTGGGGCGCGCAGA | 318 |
| 5326 | TTGACGCCTGGGGCGCGCAG | 317 |
| 5327 | CTTGACGCCTGGGGCGCGCA | 316 |
| 5328 | GCTTGACGCCTGGGGCGCGC | 315 |
| 5329 | TGCTTGACGCCTGGGGCGCG | 314 |
| 5330 | GTGCTTGACGCCTGGGGCGC | 313 |
| 5331 | GGTGCTTGACGCCTGGGGCG | 312 |
| 5332 | GGGTGCTTGACGCCTGGGGC | 311 |
| 5333 | TGGGTGCTTGACGCCTGGGG | 310 |
| 5334 | GTGGGTGCTTGACGCCTGGG | 309 |
| 5335 | TGTGGGTGCTTGACGCCTGG | 308 |
| 5336 | GTGTGGGTGCTTGACGCCTG | 307 |
| 5337 | GGTGTGGGTGCTTGACGCCT | 306 |
| 5338 | GGGTGTGGGTGCTTGACGCC | 305 |
| 5339 | AGGGTGTGGGTGCTTGACGC | 304 |
| 5340 | TAGGGTGTGGGTGCTTGACG | 303 |
| 5341 | CAGGCCGGCGCCCTAGGGGCTCC | 494 |
| 5342 | AGGCCGGCGCCCTAGGGGCT | 495 |
| 5343 | GGCCGGCGCCCTAGGGGCTC | 496 |
| 5344 | GCCGGCGCCCTAGGGGCTCC | 497 |
| 5345 | CCGGCGCCCTAGGGGCTCCT | 498 |
| 5346 | CGGCGCCCTAGGGGCTCCTC | 499 |
| 5347 | GGCGCCCTAGGGGCTCCTCC | 500 |
| 5348 | GCGCCCTAGGGGCTCCTCCT | 501 |
| 5349 | CGCCCTAGGGGCTCCTCCTC | 502 |
| 5350 | GCAGGCCGGCGCCCTAGGGG | 493 |
| 5351 | GGCAGGCCGGCGCCCTAGGG | 492 |
| 5352 | AGGCAGGCCGGCGCCCTAGG | 491 |
| 5353 | AAGGCAGGCCGGCGCCCTAG | 490 |
| 5354 | GAAGGCAGGCCGGCGCCCTA | 489 |
| 5355 | GGAAGGCAGGCCGGCGCCCT | 488 |
| 5356 | TGGAAGGCAGGCCGGCGCCC | 487 |
| 5357 | CTGGAAGGCAGGCCGGCGCC | 486 |
| 5358 | GCTGGAAGGCAGGCCGGCGC | 485 |
| 5359 | CACGCCGGCGGCGCCTTGAGCC | 58 |
| 5360 | ACGCCGGCGGCGCCTTGAGC | 59 |
| 5361 | CGCCGGCGGCGCCTTGAGCC | 60 |
| 5362 | GCCGGCGGCGCCTTGAGCCT | 61 |
| 5363 | CCGGCGGCGCCTTGAGCCTT | 62 |
| 5364 | CGGCGGCGCCTTGAGCCTTG | 63 |
| 5365 | GGCGGCGCCTTGAGCCTTGC | 64 |
| 5366 | GCGGCGCCTTGAGCCTTGCG | 65 |
| 5367 | CGGCGCCTTGAGCCTTGCGG | 66 |
| 5368 | GGCGCCTTGAGCCTTGCGGT | 67 |
| 5369 | GCGCCTTGAGCCTTGCGGTG | 68 |
| 5370 | CGCCTTGAGCCTTGCGGTGG | 69 |
| 5371 | GCCTTGAGCCTTGCGGTGGG | 70 |
| 5372 | CCTTGAGCCTTGCGGTGGGG | 71 |
| 5373 | CTTGAGCCTTGCGGTGGGGA | 72 |
| 5374 | TTGAGCCTTGCGGTGGGGAG | 73 |
| 5375 | TGAGCCTTGCGGTGGGGAGG | 74 |
| 5376 | CCACGCCGGCGGCGCCTTGA | 57 |
| 5377 | TCCACGCCGGCGGCGCCTTG | 56 |
| 5378 | GTCCACGCCGGCGGCGCCTT | 55 |
| 5379 | GGTCCACGCCGGCGGCGCCT | 54 |
| 5380 | CGGTCCACGCCGGCGGCGCC | 53 |
| 5381 | GCGGTCCACGCCGGCGGCGC | 52 |
| 5382 | CGCGGTCCACGCCGGCGGCG | 51 |
| 5383 | GCGCGGTCCACGCCGGCGGC | 50 |
| 5384 | TGCGCGGTCCACGCCGGCGG | 49 |
| 5385 | GTGCGCGGTCCACGCCGGCG | 48 |
| 5386 | CGTGCGCGGTCCACGCCGGC | 47 |
| 5387 | CCGTGCGCGGTCCACGCCGG | 46 |
| 5388 | GCCGTGCGCGGTCCACGCCG | 45 |
| 5389 | GGCCGTGCGCGGTCCACGCC | 44 |
| 5390 | AGGCCGTGCGCGGTCCACGC | 43 |
| 5391 | GAGGCCGTGCGCGGTCCACG | 42 |
| 5392 | AGAGGCCGTGCGCGGTCCAC | 41 |
| 5393 | TAGAGGCCGTGCGCGGTCCA | 40 |
| 5394 | CTAGAGGCCGTGCGCGGTCC | 39 |
| 5395 | CCTAGAGGCCGTGCGCGGTC | 38 |
| 5396 | ACCTAGAGGCCGTGCGCGGT | 37 |
| 5397 | GACCTAGAGGCCGTGCGCGG | 36 |
| 5398 | AGACCTAGAGGCCGTGCGCG | 35 |
| 5399 | GAGACCTAGAGGCCGTGCGC | 34 |
| 5400 | GGAGACCTAGAGGCCGTGCG | 33 |
| 5401 | AGGAGACCTAGAGGCCGTGC | 32 |
| 5402 | CAGGTTTCGGCCTCGCCCTCCC | 408 |
| 5403 | AGGTTTCGGCCTCGCCCTCC | 409 |
| 5404 | GGTTTCGGCCTCGCCCTCCC | 410 |
| 5405 | GTTTCGGCCTCGCCCTCCCC | 411 |
| 5406 | TTTCGGCCTCGCCCTCCCCA | 412 |
| 5407 | TTCGGCCTCGCCCTCCCCAA | 413 |
| 5408 | TCGGCCTCGCCCTCCCCAAA | 414 |
| 5409 | CGGCCTCGCCCTCCCCAAAC | 415 |
| 5410 | GGCCTCGCCCTCCCCAAACA | 416 |
| 5411 | GCCTCGCCCTCCCCAAACAG | 417 |
| 5412 | CCTCGCCCTCCCCAAACAGC | 418 |
| 5413 | CTCGCCCTCCCCAAACAGCG | 419 |
| 5414 | TCGCCCTCCCCAAACAGCGT | 420 |
| 5415 | CGCCCTCCCCAAACAGCGTC | 421 |
| 5416 | GCCCTCCCCAAACAGCGTCA | 422 |
| 5417 | CCCTCCCCAAACAGCGTCAG | 423 |
| 5418 | CCTCCCCAAACAGCGTCAGA | 424 |
| 5419 | CTCCCCAAACAGCGTCAGAT | 425 |
| 5420 | TCCCCAAACAGCGTCAGATT | 426 |
| 5421 | CCCCAAACAGCGTCAGATTA | 427 |
| 5422 | CCCAAACAGCGTCAGATTAC | 428 |
| 5423 | CCAAACAGCGTCAGATTACG | 429 |
| 5424 | CAAACAGCGTCAGATTACGC | 430 |
| 5425 | AAACAGCGTCAGATTACGCG | 431 |
| 5426 | AACAGCGTCAGATTACGCGC | 432 |
| 5427 | ACAGCGTCAGATTACGCGCA | 433 |
| 5428 | CAGCGTCAGATTACGCGCAG | 434 |
| 5429 | AGCGTCAGATTACGCGCAGA | 435 |
| 5430 | GCGTCAGATTACGCGCAGAG | 436 |
| 5431 | CGTCAGATTACGCGCAGAGG | 437 |
| 5432 | GTCAGATTACGCGCAGAGGG | 438 |
| 5433 | TCAGATTACGCGCAGAGGGA | 439 |
| 5434 | TCAGGTTTCGGCCTCGCCCT | 407 |
| 5435 | ATCAGGTTTCGGCCTCGCCC | 406 |
| 5436 | GATCAGGTTTCGGCCTCGCC | 405 |
| 5437 | GGATCAGGTTTCGGCCTCGC | 404 |
| 5438 | AGGATCAGGTTTCGGCCTCG | 403 |
| 5439 | GAGGATCAGGTTTCGGCCTC | 402 |
| 5440 | GGAGGATCAGGTTTCGGCCT | 401 |
| 5441 | TGGAGGATCAGGTTTCGGCC | 400 |
| 5442 | CTGGAGGATCAGGTTTCGGC | 399 |
| 5443 | ACTGGAGGATCAGGTTTCGG | 398 |
| 5444 | GACTGGAGGATCAGGTTTCG | 397 |
| 5445 | CATCGAGCCCGCCATCGCAGCAC | 1307 |
| 5446 | ATCGAGCCCGCCATCGCAGC | 1308 |
| 5447 | TCGAGCCCGCCATCGCAGCA | 1309 |
| 5448 | CGAGCCCGCCATCGCAGCAC | 1310 |
| 5449 | GAGCCCGCCATCGCAGCACA | 1311 |
| 5450 | AGCCCGCCATCGCAGCACAG | 1312 |
| 5451 | GCCCGCCATCGCAGCACAGA | 1313 |
| 5452 | CCCGCCATCGCAGCACAGAG | 1314 |
| 5453 | CCGCCATCGCAGCACAGAGT | 1315 |
| 5454 | CGCCATCGCAGCACAGAGTA | 1316 |
| 5455 | GCCATCGCAGCACAGAGTAG | 1317 |
| 5456 | CCATCGCAGCACAGAGTAGG | 1318 |
| 5457 | CATCGCAGCACAGAGTAGGA | 1319 |
| 5458 | CCATCGAGCCCGCCATCGCA | 1306 |
| 5459 | CCCATCGAGCCCGCCATCGC | 1305 |
| 5460 | CCCCATCGAGCCCGCCATCG | 1304 |
| 5461 | TCCCCATCGAGCCCGCCATC | 1303 |
| 5462 | ATCCCCATCGAGCCCGCCAT | 1302 |
| 5463 | TATCCCCATCGAGCCCGCCA | 1301 |
| 5464 | TTATCCCCATCGAGCCCGCC | 1300 |
| 5465 | GTTATCCCCATCGAGCCCGC | 1299 |
| 5466 | AGTTATCCCCATCGAGCCCG | 1298 |
| 5467 | GAGTTATCCCCATCGAGCCC | 1297 |
| 5468 | AGAGTTATCCCCATCGAGCC | 1296 |
| 5469 | CAGAGTTATCCCCATCGAGC | 1295 |
| 5470 | TCAGAGTTATCCCCATCGAG | 1294 |
| 5471 | GTCAGAGTTATCCCCATCGA | 1293 |
| 5472 | GGTCAGAGTTATCCCCATCG | 1292 |
| 5473 | GAGCGCCTCGACGTCGCTGCGGAAACC | 273 |
| 5474 | AGCGCCTCGACGTCGCTGCG | 274 |
| 5475 | GCGCCTCGACGTCGCTGCGG | 275 |
| 5476 | CGCCTCGACGTCGCTGCGGA | 276 |
| 5477 | GCCTCGACGTCGCTGCGGAA | 277 |
| 5478 | CCTCGACGTCGCTGCGGAAA | 278 |
| 5479 | CTCGACGTCGCTGCGGAAAC | 279 |
| 5480 | TCGACGTCGCTGCGGAAACC | 280 |
| 5481 | CGACGTCGCTGCGGAAACCT | 281 |
| 5482 | GACGTCGCTGCGGAAACCTT | 282 |
| 5483 | ACGTCGCTGCGGAAACCTTC | 283 |
| 5484 | CGTCGCTGCGGAAACCTTCT | 284 |
| 5485 | GTCGCTGCGGAAACCTTCTA | 285 |
| 5486 | TCGCTGCGGAAACCTTCTAG | 286 |
| 5487 | CGCTGCGGAAACCTTCTAGG | 287 |
| 5488 | GCTGCGGAAACCTTCTAGGG | 288 |
| 5489 | CTGCGGAAACCTTCTAGGGT | 289 |
| 5490 | TGCGGAAACCTTCTAGGGTG | 290 |
| 5491 | GCGGAAACCTTCTAGGGTGT | 291 |
| 5492 | CGGAAACCTTCTAGGGTGTG | 292 |
| 5493 | TGAGCGCCTCGACGTCGCTG | 272 |
| 5494 | ATGAGCGCCTCGACGTCGCT | 271 |
| 5495 | CATGAGCGCCTCGACGTCGC | 270 |
| 5496 | CCATGAGCGCCTCGACGTCG | 269 |
| 5497 | ACCATGAGCGCCTCGACGTC | 268 |
| 5498 | AACCATGAGCGCCTCGACGT | 267 |
| 5499 | CAACCATGAGCGCCTCGACG | 266 |
| 5500 | GCAACCATGAGCGCCTCGAC | 265 |
| 5501 | TGCAACCATGAGCGCCTCGA | 264 |
| 5502 | CTGCAACCATGAGCGCCTCG | 263 |
| 5503 | CCTGCAACCATGAGCGCCTC | 262 |
| 5504 | GCCTGCAACCATGAGCGCCT | 261 |
| 5505 | CGCCTGCAACCATGAGCGCC | 260 |
| 5506 | CCGCCTGCAACCATGAGCGC | 259 |
| 5507 | CCCGCCTGCAACCATGAGCG | 258 |
| 5508 | GCCCGCCTGCAACCATGAGC | 257 |
| 5509 | CGCCCGCCTGCAACCATGAG | 256 |
| 5510 | GCGCCCGCCTGCAACCATGA | 255 |
| 5511 | GGCGCCCGCCTGCAACCATG | 254 |
| 5512 | CGGCGCCCGCCTGCAACCAT | 253 |
| 5513 | GCGGCGCCCGCCTGCAACCA | 252 |
| 5514 | GGCGGCGCCCGCCTGCAACC | 251 |
| 5515 | CGGCGGCGCCCGCCTGCAAC | 250 |
| 5516 | ACGGCGGCGCCCGCCTGCAA | 249 |
| 5517 | AACGGCGGCGCCCGCCTGCA | 248 |
| 5518 | GAACGGCGGCGCCCGCCTGC | 247 |
| 5519 | TGAACGGCGGCGCCCGCCTG | 246 |
| 5520 | CTGAACGGCGGCGCCCGCCT | 245 |
| 5521 | ACTGAACGGCGGCGCCCGCC | 244 |
| 5522 | AACTGAACGGCGGCGCCCGC | 243 |
| 5523 | GAACTGAACGGCGGCGCCCG | 242 |
| 5524 | TGAACTGAACGGCGGCGCCC | 241 |
| 5525 | CTGAACTGAACGGCGGCGCC | 240 |
| 5526 | CCTGAACTGAACGGCGGCGC | 239 |
| 5527 | CCCTGAACTGAACGGCGGCG | 238 |
| 5528 | ACCCTGAACTGAACGGCGGC | 237 |
| 5529 | GACCCTGAACTGAACGGCGG | 236 |
| 5530 | AGACCCTGAACTGAACGGCG | 235 |
| 5531 | CAGACCCTGAACTGAACGGC | 234 |
| 5532 | TCAGACCCTGAACTGAACGG | 233 |
| 5533 | CTCAGACCCTGAACTGAACG | 232 |

Hot Zones (Relative upstream location to gene start site)
1-800
1100-1450

### Examples

Genetic Code (5′ Upstream Region)

(SEQ ID NO: 11968)

CCATCCTGGGCCATTGGGCCAGCTCCAGCCTCATCCTTGAATGGTGGGTG
TACATCGCTGGGGTCCTTGCTAGATTATATTAGGGCCTCCACACACTTTA
GTTGCTCTCCGTTTGCACAATGATTTCTATATCATTAGTACCTCTTTGTC
CACTTGCCTAATTATTTCCTTAGGATAAATTCCTAAGAAATCAAATAGCT
AGGTCAGTATATAGCACATTTTCACAGTTTGCTACTGACTAGAGATATTA
AAGGTACAAAAACAGCCAGAATTAGATGTAGAGTCGCAAGGGAGTTTTGT
TACTAAGACGTCATTTCTAATCAGGGAGAAAAAATGAGTTACGGAATAAA
CGGTATTGGAACAATAGGCTAGCCTTCTGGAGAAAAATAACCTCACTCCT
TATACAAAAGTAAATCCCAGTGGAAGCCAAGATATTAAAAAAAGATTTAA
AAAAGGAGGACAATTTTTATATGATCTTGATAAGGAGGAGGCCTTTCTAA
GCACAGTACAAAATCAAGAAGTCATGAAATAAAAGGCTGATAAGTTTGAC
TCCATGAAAATTAAAATTTTCTATGGAAAAAAATACAATAAAGTCAAAAA
TCAATCAACAAACTGGAAAATAGATTTGCAACATACATAAGAGACAGCAG
GCTAATTTTGGTATTATACATAAAAAGCTATTACAAATCATCAAACAAAA
GCTCCACAGCCAAAATGAAAAAATGAAGAGACAGTTCAAAGACAAACAAA
TGTAGATAATTGTTTAACTTAAGGAAAGGTCAAAATATTTGGCAACTCTG
TGTTGGCCTGGGTGTGACCATGAGCGGTAGTTGCCAGTGGTATTCACAAA
TATACCCTTTCTCCTCCTTCTGGGCACTTGGTGTGATTGCAGTTTCCTAC
ATTTGACCTTAGGTGTGGTCATGTGTCTCGCTTAGGAGAAGGAAATGTGA
ACGGAAGTGTTGTGGGTCACTTTTGTGTGGAAGCTGCATGGAGTCACCTC
TTTGCTTTCCGTCAGCCTCAGTGTTCAGCAATGTTCTGAATGATGGTTGC
TTTATCAGTCTGGTTCTGGGGTGAGGGTAATGAAGTAGTGAAGCAAAGTC
CTTGATGGACATGTGAGGTGAGACAGAAATAAACCTTTGACATTTCAAGC
CCCTGAGATTTGGGGCGTGCATGTGCTGGAAGCAGAACCTAGCCTATTCT
GATGGATTCCTCCAGCACTGCTCGTGGGAAGACATCATCAATATAGAGCA
TTCATGTGCCCTTTATCTCGAAATTCCACTTCCAGGAATTTATGAAACAG
ATACTCTCACATGTGCAAACAGCTATGGATAAGGACAGACATGGCAACTT
GGATTGCAATAGCAAAAGACAGAAACAACCAACGGAAACACCAACCAATA
GGAAATTGGCTAAAGACATTGTGAAACATACATAGAATGAAATAATCTGC
AACCAGAAAAATAAGGCAGTAGATGTATGTGTACCAGTGTGGTTTTTATT
CCGAGATTAGGGCTAGGTTAAGACGTCAGATTAAGTTGTCCCTCTCCACC
CCACCAATATAAATAAAAAGTTAAAAGTAAATCATAAACTATTTTTACAA
TTTTAAAAAGTGGGTTAAAGAGCCCATCCAAGTAGTTTTATAAAAGTAGA
CTATCTCCGAAAAGATACCCAATAAATAGGTATATTACTTTCCTGGGGCT
GTTATAACAAGTTTCTACAAATTTGCTGGCTTCAAATAACAAAAACGTAT
TCTCTTGCAGTTATGGAAGCCAGAAGTATGGAATGAAGGGTTGCAGGGTG
GTGCCCTCTCCCAAAGCTCTAGGGGAGGAACATTCCTTGCTTCTTCCAGC
TCCTTTGGGGGCTCCTGGCATTCCTTGGCTTATGTCGGCACAGCTCTAAT
CGGCGCCTCCATTGTTACATAGGTGTTTCTGTGTCTCAAGTATCTCTCCC
CTTTCTCTTCTGATATCAGTCATTGGATTTAGGGACCATCCTAAACCCAG
GATAATCTCCTCATGAGATCCTTAGGTCAATTACATCTGCAAAGATCTCA
TTTCCAAATAAGGTCACATTCAAAAGTACCAGGGGTTAGTCTTAGACTTA
TCTTTTTGGGGGACACGATTCAACCCACTACCGTGGGTAACAGTGGTTTT
CCCTCAGAAGGTGGTGGTTCAGGAGTGGGAGGAAGATGAACTTTTCACTG
TATATCCTTTCAAACTATTCACGTTTTAAAAAAAACATTTTCATGTAAAT
TTAAAAAAATTGAACATTCACACAAAAAGATGCCCCCTCCCTTGCAAAAA
AGAGTATGCCCGTTCAAAATGTTGAAATGTACACTCACAGCAATGGTGGC
TGCAGACTCCAAGTTTCTGAGGTTGGAGAAGGTAGCCAGGGAGCATAAAA
GTGAGTTCTATCTACTCATTCAGTCTATGAGGGGAAGGCAATGGCTAGAA
AAGCATTTTGAGGGACAGTAAAAGTGGCATTTTTAGAGGGAGGAAGCCTT
GAGGATGCTTGTGGGGTGAAGGGAAAGAATAACTCAGGAAGAGGCATTTA
GGGATAAGAGGAGGAGAGGAGATAGTGGAGGTAGGTGATCCCTGCGGAGG
CCAGATTGGGGCAGGGGAGTGTCAGCTGAGTATAAGAGGATGGTCCCCTC
TGCCCTGAAGGAGGAAGGCAGGAGGGGAAAAGGATGGGTGTTGACCCAGA
AAGCACTTGTGGTGGAGGGGAGGCCCCAGAAGAGGCTTCTGACTTACCCT
GATTGCTGGTACCTCTCAGGGGAGCTGGCTGCTTATTTGCTGGCCAGGGT
GTGGGGGAACCCATTTGAGAAGAGGGAGAAGGTGACACAATTCCTTTGGG
CAACTTATGGGAGGGGTAATTGGTGAGGGATGAAAGCCCTGCCAAGTGGC
AGGAGGCCCAGCTGGGGCTGCCCCTCATAAGAGTGCAGTGGAGGATATGG
GATGAGAAGTGACTGCCCCTCTGGTTCCATCTGTCGCAGAGCCCAGGGTG
CTTCCTTCCTCCCCCACCTCCCTCAGAACACACCCACTGCATGCTGGACA
GCAGCCCCCTTCCTGGGCCTGGGGACATCCATGTCCCTCTGTGCACAGGC
TTCATCATTCTCTGGGTGCACGGTAACGACCCCGGTAGGTGAGAGGCCAA
GGTCCCAAAGGGGAGCAGCAGGGAAAGTTAGCTCCCATCTATTCTTGCTC
CAGGGGAGGCCTTTGATGAGGAAGCTGCCAAAAGCACATTGCAAATACAA
TTCCAATTACAGGCAACAGGAAGGAGAACCACCTCTGCCACCTCTGTCAG
CAAACCATGAGCTCCTACTCTGTGCTGCGATGGCGGGCTCGATGGGGATA
ACTCTGACCTTACCTCATGGAGTCACTGTCAACCCACTGGTTGCACTGTC
TTTGTGCACTGGCTCTCTGGAGTGAGGTCTTTGCAAACAAAGTGGAAAGA
GCATCAACTTTGGACTCCAGCACCTAGATTCAGAGCAGGCCATTTCACTC
GGAATCTGCTGTGCATCTGCAAGGGAGGATCATAAATTCGCCTTTGTTTC
TTCCCAGTATCGACAGCCCTTCCAGAAAGAGCAAGCCTCATGTCATGCCA
CATGTACAATCTGAGGCCAGGAGCTCTCTTTCCCCTTTTCATCCTCCTGC
CTGGTACACAATAGGTGTTTACTGGATGCTTGTCCAGTTGATTTCTTGAA
CATGGTGTGTAAAAGGAATCTTTGCAAATTGAATCTTCTGGAAAGCTGAG
CTTGTGCCTACCATAGAATTCTGAATGTACCTATATGACGTCTTTGCAAA
CTTAAAACCTGAATCTTTGTAGTATAAATCCCTTGAAATGCATGTAGGCT
GGACATCAAAAGCAAGCAATCTCTTCAAGGAGCAGCTAGTTGGTAAGGTC
AGTGTGCAGGGTGCATAAAGGGCAGAGGCCGGAGGGGGTCCAGGCTAAGT
TTAGAAGGCTGCCAGGTTAAGGCCAGTGGAAAGAATTCGGTGGGCAGCGA
GGAGTCCACAGTAGGATTGATTCAGAAGTCTCACTGGTCAGCAGGAGACA
AGGTGGACCCAGGAAACACTGAAAAGGTGGGCCCGGCAGAACTTGGAGTC
TGGCATCCCACGCAGGGTGAGAGGCGGGAGAGGAGGAGCCCCTAGGGCGC
CGGCCTGCCTTCCAGCCCAGTTAGGATTTGGGAGTTTTTTCTTCCCTCTG
CGCGTAATCTGACGCTGTTTGGGGAGGGCGAGGCCGAAACCTGATCCTCC
AGTCCGGGGGTTCCGTTAATGTTTAATCAGATAGGATCGTCCGATGGGGC
TCTGGTGGCGTGATCTGCGCGCCCCAGGCGTCAAGCACCCACACCCTAGA
AGGTTTCCGCAGCGACGTCGAGGCGCTCATGGTTGCAGGCGGGCGCCGCC
GTTCAGTTCAGGGTCTGAGCCTGGAGGAGTGAGCCAGGCAGTGAGACTGG
CTCGGGCGGGCCGGGACGCGTCGTTGCAGCAGCGGCTCCCAGCTCCCAGC
CAGGATTCCGCGCGCCCCTTCACGCGCCCTGCTCCTGAACTTCAGCTCCT
GCACAGTCCTCCCCACCGCAAGGCTCAAGGCGCCGCCGGCGTGGACCGCG
CACGGCCTCTAGGTCTCCTCGCCAGGACAGCAACCTCTCCCCTGGCCCTC
ATG

20) MEK1. MEK1 (MAP2K1) Mitogen-activated protein kinase kinase 1. Dual specificity protein kinases act as an essential component of the MAP kinase signal transduction pathway and serves as an integration point for multiple biochemical signals. MEK1 and MEK2 are members of the dual specificity protein kinase family, which act as a mitogen-activated protein (MAP) kinase kinases and as extracellular signal-regulated kinases (ERKs). Binding of extracellular ligands such as growth factors, cytokines and hormones to their cell-surface receptors activates RAS and this initiates RAF1 activation. RAF1 then further activates the dual-specificity protein kinases MAP2K1/MEK1 and MAP2K2/MEK2. Both MAP2K1/MEK1 and MAP2K2/MEK2 function specifically in the MAPK/ERK cascade, and catalyze the concomitant phosphorylation of a threonine and a tyrosine residue in a Thr-Glu-Tyr sequence located in the extracellular signal-regulated kinases MAPK3/ERK1 and MAPK1/ERK2, leading to their activation and further transduction of the signal within the MAPK/ERK cascade. Depending on the cellular context, this pathway mediates diverse biological functions such as cell growth and proliferation, adhesion, survival and differentiation, predominantly through the regulation of transcription, metabolism and cytoskeletal rearrangements (reviewed by Roberts and Der; 2007 Oncogene 26, 3291-3310).

Genetic alterations that activate the mitogen-activated protein kinase (MAP kinase) pathway occur commonly in cancer. For example, the majority of melanomas harbor mutations in the BRAF oncogene, which confers enhanced sensitivity to pharmacologic MAP kinase inhibition (e.g., RAF or MEK inhibitors). Most mutations conferring resistance to MEK inhibition in vitro populated the allosteric drug binding pocket or alpha-helix C and showed robust (approximately 100-fold) resistance to allosteric MEK inhibition (reviewed in Emery et al, 2009; Proc Natl Acad Sci.; 106(48):20411-20416). Other mutations affected MEK1 codons located within or abutting the N-terminal negative regulatory helix (helix A), which also undergo gain-of-function germline mutations in cardiofaciocutaneous (CFC) syndrome. One target of the MAPK/ERK cascade is peroxisome proliferator-activated receptor gamma (PPARG), a nuclear receptor that promotes differentiation and apoptosis. MAP2K1/MEK1 has been shown to export PPARG from the nucleus. The MAPK/ERK cascade is also involved in the regulation of endosomal dynamics, including lysosome processing and endosome cycling through the perinuclear recycling compartment (PNRC), as well as in the fragmentation of the Golgi apparatus during mitosis.

Protein: MEK1 Gene: MAP2K1 (Homo sapiens, chromosome 15, 66679211-66783882 [NCBI Reference Sequence: NC_{—}000015.9]; start site location: 66679686; strand: positive)

Gene Identification

| | |
|---|---|
| GeneID | 5604 |
| HGNC | 6840 |
| HPRD | 01469 |
| MIM | 176872 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 5534 | | CAAGTCCGGGCCGCGGGCCCCGGGGC | 93 |
| 5716 | MEK1_2 | GCGCCCCGCGCGGTCCCGTCAGCGC | 133 |
| 5898 | | GCGGAGCGGGCTGAACGTGCG | 249 |
| 5900 | | GACTGGAGGCCGGGGGAGGGGCGGGG | 433 |
| 5901 | | GACCCGGGTAACGCGCTTCCAAC | 5 |
| 5924 | MEK1_1 | CACTCGGCTCCGCCCCTATTGC | 507 |
| 6000 | | TACGTCACGGGAGCGCGGCGCAC | 578 |
| 6077 | | GTCGCGGACGCCGTGGCGCCCTCTGTC | 619 |
| 6154 | | CACTCGCCGTCATGCCCGGATCC | 1183 |

Target Shift Sequences

| | | Relative |
|---|---|---|
| | | upstream |
| | | location |
| Sequence | | to gene |
| ID No: | Sequence (5′-3′) | start site |
| 5534 | CAAGTCCGGGCCGCGGGCCCCGGGGC | 93 |
| 5535 | AAGTCCGGGCCGCGGGCCCC | 94 |
| 5536 | AGTCCGGGCCGCGGGCCCCG | 95 |
| 5537 | GTCCGGGCCGCGGGCCCCGG | 96 |
| 5538 | TCCGGGCCGCGGGCCCCGGG | 97 |
| 5539 | CCGGGCCGCGGGCCCCGGGG | 98 |
| 5540 | CGGGCCGCGGGCCCCGGGGC | 99 |
| 5541 | GGGCCGCGGGCCCCGGGGCT | 100 |
| 5542 | GGCCGCGGGCCCCGGGGCTG | 101 |
| 5543 | GCCGCGGGCCCCGGGGCTGC | 102 |
| 5544 | CCGCGGGCCCCGGGGCTGCC | 103 |
| 5545 | CGCGGGCCCCGGGGCTGCCT | 104 |
| 5546 | GCGGGCCCCGGGGCTGCCTT | 105 |
| 5547 | CGGGCCCCGGGGCTGCCTTC | 106 |
| 5548 | GGGCCCCGGGGCTGCCTTCA | 107 |
| 5549 | GGCCCCGGGGCTGCCTTCAG | 108 |
| 5550 | GCCCCGGGGCTGCCTTCAGC | 109 |
| 5551 | CCCCGGGGCTGCCTTCAGCG | 110 |
| 5552 | CCCGGGGCTGCCTTCAGCGG | 111 |
| 5553 | CCGGGGCTGCCTTCAGCGGG | 112 |
| 5554 | CGGGGCTGCCTTCAGCGGGT | 113 |
| 5555 | GGGGCTGCCTTCAGCGGGTG | 114 |
| 5556 | GGGCTGCCTTCAGCGGGTGC | 115 |
| 5557 | GGCTGCCTTCAGCGGGTGCG | 116 |
| 5558 | GCTGCCTTCAGCGGGTGCGC | 117 |
| 5559 | CTGCCTTCAGCGGGTGCGCC | 118 |
| 5560 | TGCCTTCAGCGGGTGCGCCC | 119 |
| 5561 | GCCTTCAGCGGGTGCGCCCC | 120 |
| 5562 | CCTTCAGCGGGTGCGCCCCG | 121 |
| 5563 | CTTCAGCGGGTGCGCCCCGC | 122 |
| 5564 | TTCAGCGGGTGCGCCCCGCG | 123 |
| 5565 | TCAGCGGGTGCGCCCCGCGC | 124 |
| 5566 | CAGCGGGTGCGCCCCGCGCG | 125 |
| 5567 | AGCGGGTGCGCCCCGCGCGG | 126 |
| 5568 | GCGGGTGCGCCCCGCGCGGT | 127 |
| 5569 | CGGGTGCGCCCCGCGCGGTC | 128 |
| 5570 | GGGTGCGCCCCGCGCGGTCC | 129 |
| 5571 | GGTGCGCCCCGCGCGGTCCC | 130 |
| 5572 | GTGCGCCCCGCGCGGTCCCG | 131 |
| 5573 | TGCGCCCCGCGCGGTCCCGT | 132 |
| 5574 | GCGCCCCGCGCGGTCCCGTC | 133 |
| 5575 | CGCCCCGCGCGGTCCCGTCA | 134 |
| 5576 | GCCCCGCGCGGTCCCGTCAG | 135 |
| 5577 | CCCCGCGCGGTCCCGTCAGC | 136 |
| 5578 | CCCGCGCGGTCCCGTCAGCG | 137 |
| 5579 | CCGCGCGGTCCCGTCAGCGC | 138 |
| 5580 | CGCGCGGTCCCGTCAGCGCC | 139 |
| 5581 | GCGCGGTCCCGTCAGCGCCG | 140 |
| 5582 | CGCGGTCCCGTCAGCGCCGA | 141 |
| 5583 | GCGGTCCCGTCAGCGCCGAG | 142 |
| 5584 | CGGTCCCGTCAGCGCCGAGG | 143 |
| 5585 | GGTCCCGTCAGCGCCGAGGG | 144 |
| 5586 | GTCCCGTCAGCGCCGAGGGG | 145 |
| 5587 | TCCCGTCAGCGCCGAGGGGC | 146 |
| 5588 | CCCGTCAGCGCCGAGGGGCC | 147 |
| 5589 | CCGTCAGCGCCGAGGGGCCG | 148 |
| 5590 | CGTCAGCGCCGAGGGGCCGG | 149 |
| 5591 | GTCAGCGCCGAGGGGCCGGT | 150 |
| 5592 | TCAGCGCCGAGGGGCCGGTA | 151 |
| 5593 | CAGCGCCGAGGGGCCGGTAG | 152 |
| 5594 | AGCGCCGAGGGGCCGGTAGC | 153 |
| 5595 | GCGCCGAGGGGCCGGTAGCG | 154 |
| 5596 | CGCCGAGGGGCCGGTAGCGG | 155 |
| 5597 | GCCGAGGGGCCGGTAGCGGT | 156 |
| 5598 | CCGAGGGGCCGGTAGCGGTC | 157 |
| 5599 | CGAGGGGCCGGTAGCGGTCT | 158 |
| 5600 | GAGGGGCCGGTAGCGGTCTC | 159 |
| 5601 | AGGGGCCGGTAGCGGTCTCA | 160 |
| 5602 | GGGGCCGGTAGCGGTCTCAG | 161 |
| 5603 | GGGCCGGTAGCGGTCTCAGT | 162 |
| 5604 | GGCCGGTAGCGGTCTCAGTG | 163 |
| 5605 | GCCGGTAGCGGTCTCAGTGG | 164 |
| 5606 | CCGGTAGCGGTCTCAGTGGA | 165 |
| 5607 | CGGTAGCGGTCTCAGTGGAC | 166 |
| 5608 | GGTAGCGGTCTCAGTGGACC | 167 |
| 5609 | GTAGCGGTCTCAGTGGACCC | 168 |
| 5610 | TAGCGGTCTCAGTGGACCCC | 169 |
| 5611 | AGCGGTCTCAGTGGACCCCC | 170 |
| 5612 | GCGGTCTCAGTGGACCCCCG | 171 |
| 5613 | CGGTCTCAGTGGACCCCCGC | 172 |
| 5614 | GGTCTCAGTGGACCCCCGCC | 173 |
| 5615 | GTCTCAGTGGACCCCCGCCC | 174 |
| 5616 | TCTCAGTGGACCCCCGCCCC | 175 |
| 5617 | CTCAGTGGACCCCCGCCCCA | 176 |
| 5618 | TCAGTGGACCCCCGCCCCAC | 177 |
| 5619 | CAGTGGACCCCCGCCCCACC | 178 |
| 5620 | AGTGGACCCCCGCCCCACCC | 179 |
| 5621 | GTGGACCCCCGCCCCACCCG | 180 |
| 5622 | TGGACCCCCGCCCCACCCGC | 181 |
| 5623 | GGACCCCCGCCCCACCCGCC | 182 |
| 5624 | GACCCCCGCCCCACCCGCCC | 183 |
| 5625 | ACCCCCGCCCCACCCGCCCG | 184 |
| 5626 | CCCCCGCCCCACCCGCCCGG | 185 |
| 5627 | CCCCGCCCCACCCGCCCGGG | 186 |
| 5628 | CCCGCCCCACCCGCCCGGGA | 187 |
| 5629 | CCGCCCCACCCGCCCGGGAC | 188 |
| 5630 | CGCCCCACCCGCCCGGGACT | 189 |
| 5631 | GCCCCACCCGCCCGGGACTC | 190 |
| 5632 | CCCCACCCGCCCGGGACTCG | 191 |
| 5633 | CCCACCCGCCCGGGACTCGG | 192 |
| 5634 | CCACCCGCCCGGGACTCGGC | 193 |
| 5635 | CACCCGCCCGGGACTCGGCT | 194 |
| 5636 | ACCCGCCCGGGACTCGGCTT | 195 |
| 5637 | CCCGCCCGGGACTCGGCTTC | 196 |
| 5638 | CCGCCCGGGACTCGGCTTCG | 197 |
| 5639 | CGCCCGGGACTCGGCTTCGC | 198 |
| 5640 | GCCCGGGACTCGGCTTCGCG | 199 |
| 5641 | CCCGGGACTCGGCTTCGCGC | 200 |
| 5642 | CCGGGACTCGGCTTCGCGCG | 201 |
| 5643 | CGGGACTCGGCTTCGCGCGC | 202 |
| 5644 | GGGACTCGGCTTCGCGCGCA | 203 |
| 5645 | GGACTCGGCTTCGCGCGCAG | 204 |
| 5646 | GACTCGGCTTCGCGCGCAGA | 205 |
| 5647 | ACTCGGCTTCGCGCGCAGAG | 206 |
| 5648 | CTCGGCTTCGCGCGCAGAGA | 207 |
| 5649 | TCGGCTTCGCGCGCAGAGAG | 208 |
| 5650 | CGGCTTCGCGCGCAGAGAGC | 209 |
| 5651 | GGCTTCGCGCGCAGAGAGCC | 210 |
| 5652 | GCTTCGCGCGCAGAGAGCCG | 211 |
| 5653 | CTTCGCGCGCAGAGAGCCGA | 212 |
| 5654 | TTCGCGCGCAGAGAGCCGAA | 213 |
| 5655 | TCGCGCGCAGAGAGCCGAAA | 214 |
| 5656 | CCAAGTCCGGGCCGCGGGCC | 92 |
| 5657 | ACCAAGTCCGGGCCGCGGGC | 91 |
| 5658 | GACCAAGTCCGGGCCGCGGG | 90 |
| 5659 | GGACCAAGTCCGGGCCGCGG | 89 |
| 5660 | AGGACCAAGTCCGGGCCGCG | 88 |
| 5661 | CAGGACCAAGTCCGGGCCGC | 87 |
| 5662 | GCAGGACCAAGTCCGGGCCG | 86 |
| 5663 | CGCAGGACCAAGTCCGGGCC | 85 |
| 5664 | GCGCAGGACCAAGTCCGGGC | 84 |
| 5665 | TGCGCAGGACCAAGTCCGGG | 83 |
| 5666 | CTGCGCAGGACCAAGTCCGG | 82 |
| 5667 | GCTGCGCAGGACCAAGTCCG | 81 |
| 5668 | CGCTGCGCAGGACCAAGTCC | 80 |
| 5669 | CCGCTGCGCAGGACCAAGTC | 79 |
| 5670 | CCCGCTGCGCAGGACCAAGT | 78 |
| 5671 | GCCCGCTGCGCAGGACCAAG | 77 |
| 5672 | CGCCCGCTGCGCAGGACCAA | 76 |
| 5673 | GCGCCCGCTGCGCAGGACCA | 75 |
| 5674 | CGCGCCCGCTGCGCAGGACC | 74 |
| 5675 | CCGCGCCCGCTGCGCAGGAC | 73 |
| 5676 | CCCGCGCCCGCTGCGCAGGA | 72 |
| 5677 | CCCCGCGCCCGCTGCGCAGG | 71 |
| 5678 | GCCCCGCGCCCGCTGCGCAG | 70 |
| 5679 | TGCCCCGCGCCCGCTGCGCA | 69 |
| 5680 | CTGCCCCGCGCCCGCTGCGC | 68 |
| 5681 | GCTGCCCCGCGCCCGCTGCG | 67 |
| 5682 | CGCTGCCCCGCGCCCGCTGC | 66 |
| 5683 | GCGCTGCCCCGCGCCCGCTG | 65 |
| 5684 | TGCGCTGCCCCGCGCCCGCT | 64 |
| 5685 | CTGCGCTGCCCCGCGCCCGC | 63 |
| 5686 | GCTGCGCTGCCCCGCGCCCG | 62 |
| 5687 | CGCTGCGCTGCCCCGCGCCC | 61 |
| 5688 | CCGCTGCGCTGCCCCGCGCC | 60 |
| 5689 | CCCGCTGCGCTGCCCCGCGC | 59 |
| 5690 | TCCCGCTGCGCTGCCCCGCG | 58 |
| 5691 | CTCCCGCTGCGCTGCCCCGC | 57 |
| 5692 | CCTCCCGCTGCGCTGCCCCG | 56 |
| 5693 | TCCTCCCGCTGCGCTGCCCC | 55 |
| 5694 | TTCCTCCCGCTGCGCTGCCC | 54 |
| 5695 | CTTCCTCCCGCTGCGCTGCC | 53 |
| 5696 | GCTTCCTCCCGCTGCGCTGC | 52 |
| 5697 | CGCTTCCTCCCGCTGCGCTG | 51 |
| 5698 | TCGCTTCCTCCCGCTGCGCT | 50 |
| 5699 | CTCGCTTCCTCCCGCTGCGC | 49 |
| 5700 | TCTCGCTTCCTCCCGCTGCG | 48 |
| 5701 | CTCTCGCTTCCTCCCGCTGC | 47 |
| 5702 | CCTCTCGCTTCCTCCCGCTG | 46 |
| 5703 | ACCTCTCGCTTCCTCCCGCT | 45 |
| 5704 | CACCTCTCGCTTCCTCCCGC | 44 |
| 5705 | GCACCTCTCGCTTCCTCCCG | 43 |
| 5706 | AGCACCTCTCGCTTCCTCCC | 42 |
| 5707 | CAGCACCTCTCGCTTCCTCC | 41 |
| 5708 | GCAGCACCTCTCGCTTCCTC | 40 |
| 5709 | GGCAGCACCTCTCGCTTCCT | 39 |
| 5710 | GGGCAGCACCTCTCGCTTCC | 38 |
| 5711 | AGGGCAGCACCTCTCGCTTC | 37 |
| 5712 | GAGGGCAGCACCTCTCGCTT | 36 |
| 5713 | GGAGGGCAGCACCTCTCGCT | 35 |
| 5714 | GGGAGGGCAGCACCTCTCGC | 34 |
| 5715 | GGGGAGGGCAGCACCTCTCG | 33 |
| 5716 | GCGCCCCGCGCGGTCCCGTCAGCGC | 133 |
| 5717 | CGCCCCGCGCGGTCCCGTCA | 134 |
| 5718 | GCCCCGCGCGGTCCCGTCAG | 135 |
| 5719 | CCCCGCGCGGTCCCGTCAGC | 136 |
| 5720 | CCCGCGCGGTCCCGTCAGCG | 137 |
| 5721 | CCGCGCGGTCCCGTCAGCGC | 138 |
| 5722 | CGCGCGGTCCCGTCAGCGCC | 139 |
| 5723 | GCGCGGTCCCGTCAGCGCCG | 140 |
| 5724 | CGCGGTCCCGTCAGCGCCGA | 141 |
| 5725 | GCGGTCCCGTCAGCGCCGAG | 142 |
| 5726 | CGGTCCCGTCAGCGCCGAGG | 143 |
| 5727 | GGTCCCGTCAGCGCCGAGGG | 144 |
| 5728 | GTCCCGTCAGCGCCGAGGGG | 145 |
| 5729 | TCCCGTCAGCGCCGAGGGGC | 146 |
| 5730 | CCCGTCAGCGCCGAGGGGCC | 147 |
| 5731 | CCGTCAGCGCCGAGGGGCCG | 148 |
| 5732 | CGTCAGCGCCGAGGGGCCGG | 149 |
| 5733 | GTCAGCGCCGAGGGGCCGGT | 150 |
| 5734 | TCAGCGCCGAGGGGCCGGTA | 151 |
| 5735 | CAGCGCCGAGGGGCCGGTAG | 152 |
| 5736 | AGCGCCGAGGGGCCGGTAGC | 153 |
| 5737 | GCGCCGAGGGGCCGGTAGCG | 154 |
| 5738 | CGCCGAGGGGCCGGTAGCGG | 155 |
| 5739 | GCCGAGGGGCCGGTAGCGGT | 156 |
| 5740 | CCGAGGGGCCGGTAGCGGTC | 157 |
| 5741 | CGAGGGGCCGGTAGCGGTCT | 158 |
| 5742 | GAGGGGCCGGTAGCGGTCTC | 159 |
| 5743 | AGGGGCCGGTAGCGGTCTCA | 160 |
| 5744 | GGGGCCGGTAGCGGTCTCAG | 161 |
| 5745 | GGGCCGGTAGCGGTCTCAGT | 162 |
| 5746 | GGCCGGTAGCGGTCTCAGTG | 163 |
| 5747 | GCCGGTAGCGGTCTCAGTGG | 164 |
| 5748 | CCGGTAGCGGTCTCAGTGGA | 165 |
| 5749 | CGGTAGCGGTCTCAGTGGAC | 166 |
| 5750 | GGTAGCGGTCTCAGTGGACC | 167 |
| 5751 | GTAGCGGTCTCAGTGGACCC | 168 |
| 5752 | TAGCGGTCTCAGTGGACCCC | 169 |
| 5753 | AGCGGTCTCAGTGGACCCCC | 170 |
| 5754 | GCGGTCTCAGTGGACCCCCG | 171 |
| 5755 | CGGTCTCAGTGGACCCCCGC | 172 |
| 5756 | GGTCTCAGTGGACCCCCGCC | 173 |
| 5757 | GTCTCAGTGGACCCCCGCCC | 174 |
| 5758 | TCTCAGTGGACCCCCGCCCC | 175 |
| 5759 | CTCAGTGGACCCCCGCCCCA | 176 |
| 5760 | TCAGTGGACCCCCGCCCCAC | 177 |
| 5761 | CAGTGGACCCCCGCCCCACC | 178 |
| 5762 | AGTGGACCCCCGCCCCACCC | 179 |
| 5763 | GTGGACCCCCGCCCCACCCG | 180 |
| 5764 | TGGACCCCCGCCCCACCCGC | 181 |
| 5765 | GGACCCCCGCCCCACCCGCC | 182 |
| 5766 | GACCCCCGCCCCACCCGCCC | 183 |
| 5767 | ACCCCCGCCCCACCCGCCCG | 184 |
| 5768 | CCCCCGCCCCACCCGCCCGG | 185 |
| 5769 | CCCCGCCCCACCCGCCCGGG | 186 |
| 5770 | CCCGCCCCACCCGCCCGGGA | 187 |
| 5771 | CCGCCCCACCCGCCCGGGAC | 188 |
| 5772 | CGCCCCACCCGCCCGGGACT | 189 |
| 5773 | GCCCCACCCGCCCGGGACTC | 190 |
| 5774 | CCCCACCCGCCCGGGACTCG | 191 |
| 5775 | CCCACCCGCCCGGGACTCGG | 192 |
| 5776 | CCACCCGCCCGGGACTCGGC | 193 |
| 5777 | CACCCGCCCGGGACTCGGCT | 194 |
| 5778 | ACCCGCCCGGGACTCGGCTT | 195 |
| 5779 | CCCGCCCGGGACTCGGCTTC | 196 |
| 5780 | CCGCCCGGGACTCGGCTTCG | 197 |
| 5781 | CGCCCGGGACTCGGCTTCGC | 198 |
| 5782 | GCCCGGGACTCGGCTTCGCG | 199 |
| 5783 | CCCGGGACTCGGCTTCGCGC | 200 |
| 5784 | CCGGGACTCGGCTTCGCGCG | 201 |
| 5785 | CGGGACTCGGCTTCGCGCGC | 202 |
| 5786 | GGGACTCGGCTTCGCGCGCA | 203 |
| 5787 | GGACTCGGCTTCGCGCGCAG | 204 |
| 5788 | GACTCGGCTTCGCGCGCAGA | 205 |
| 5789 | ACTCGGCTTCGCGCGCAGAG | 206 |
| 5790 | CTCGGCTTCGCGCGCAGAGA | 207 |
| 5791 | TCGGCTTCGCGCGCAGAGAG | 208 |
| 5792 | CGGCTTCGCGCGCAGAGAGC | 209 |
| 5793 | GGCTTCGCGCGCAGAGAGCC | 210 |
| 5794 | GCTTCGCGCGCAGAGAGCCG | 211 |
| 5795 | CTTCGCGCGCAGAGAGCCGA | 212 |
| 5796 | TTCGCGCGCAGAGAGCCGAA | 213 |
| 5797 | TCGCGCGCAGAGAGCCGAAA | 214 |
| 5798 | TGCGCCCCGCGCGGTCCCGT | 132 |
| 5799 | GTGCGCCCCGCGCGGTCCCG | 131 |
| 5800 | GGTGCGCCCCGCGCGGTCCC | 130 |
| 5801 | GGGTGCGCCCCGCGCGGTCC | 129 |
| 5802 | CGGGTGCGCCCCGCGCGGTC | 128 |
| 5803 | GCGGGTGCGCCCCGCGCGGT | 127 |
| 5804 | AGCGGGTGCGCCCCGCGCGG | 126 |
| 5805 | CAGCGGGTGCGCCCCGCGCG | 125 |
| 5806 | TCAGCGGGTGCGCCCCGCGC | 124 |
| 5807 | TTCAGCGGGTGCGCCCCGCG | 123 |
| 5808 | CTTCAGCGGGTGCGCCCCGC | 122 |
| 5809 | CCTTCAGCGGGTGCGCCCCG | 121 |
| 5810 | GCCTTCAGCGGGTGCGCCCC | 120 |
| 5811 | TGCCTTCAGCGGGTGCGCCC | 119 |
| 5812 | CTGCCTTCAGCGGGTGCGCC | 118 |
| 5813 | GCTGCCTTCAGCGGGTGCGC | 117 |
| 5814 | GGCTGCCTTCAGCGGGTGCG | 116 |
| 5815 | GGGCTGCCTTCAGCGGGTGC | 115 |
| 5816 | GGGGCTGCCTTCAGCGGGTG | 114 |
| 5817 | CGGGGCTGCCTTCAGCGGGT | 113 |
| 5818 | CCGGGGCTGCCTTCAGCGGG | 112 |
| 5819 | CCCGGGGCTGCCTTCAGCGG | 111 |
| 5820 | CCCCGGGGCTGCCTTCAGCG | 110 |
| 5821 | GCCCCGGGGCTGCCTTCAGC | 109 |
| 5822 | GGCCCCGGGGCTGCCTTCAG | 108 |
| 5823 | GGGCCCCGGGGCTGCCTTCA | 107 |
| 5824 | CGGGCCCCGGGGCTGCCTTC | 106 |
| 5825 | GCGGGCCCCGGGGCTGCCTT | 105 |
| 5826 | CGCGGGCCCCGGGGCTGCCT | 104 |
| 5827 | CCGCGGGCCCCGGGGCTGCC | 103 |
| 5828 | GCCGCGGGCCCCGGGGCTGC | 102 |
| 5829 | GGCCGCGGGCCCCGGGGCTG | 101 |
| 5830 | GGGCCGCGGGCCCCGGGGCT | 100 |
| 5831 | CGGGCCGCGGGCCCCGGGGC | 99 |
| 5832 | CCGGGCCGCGGGCCCCGGGG | 98 |
| 5833 | TCCGGGCCGCGGGCCCCGGG | 97 |
| 5834 | GTCCGGGCCGCGGGCCCCGG | 96 |
| 5835 | AGTCCGGGCCGCGGGCCCCG | 95 |
| 5836 | AAGTCCGGGCCGCGGGCCCC | 94 |
| 5837 | CAAGTCCGGGCCGCGGGCCC | 93 |
| 5838 | CCAAGTCCGGGCCGCGGGCC | 92 |
| 5839 | ACCAAGTCCGGGCCGCGGGC | 91 |
| 5840 | GACCAAGTCCGGGCCGCGGG | 90 |
| 5841 | GGACCAAGTCCGGGCCGCGG | 89 |
| 5842 | AGGACCAAGTCCGGGCCGCG | 88 |
| 5843 | CAGGACCAAGTCCGGGCCGC | 87 |
| 5844 | GCAGGACCAAGTCCGGGCCG | 86 |
| 5845 | CGCAGGACCAAGTCCGGGCC | 85 |
| 5846 | GCGCAGGACCAAGTCCGGGC | 84 |
| 5847 | TGCGCAGGACCAAGTCCGGG | 83 |
| 5848 | CTGCGCAGGACCAAGTCCGG | 82 |
| 5849 | GCTGCGCAGGACCAAGTCCG | 81 |
| 5850 | CGCTGCGCAGGACCAAGTCC | 80 |
| 5851 | CCGCTGCGCAGGACCAAGTC | 79 |
| 5852 | CCCGCTGCGCAGGACCAAGT | 78 |
| 5853 | GCCCGCTGCGCAGGACCAAG | 77 |
| 5854 | CGCCCGCTGCGCAGGACCAA | 76 |
| 5855 | GCGCCCGCTGCGCAGGACCA | 75 |
| 5856 | CGCGCCCGCTGCGCAGGACC | 74 |
| 5857 | CCGCGCCCGCTGCGCAGGAC | 73 |
| 5858 | CCCGCGCCCGCTGCGCAGGA | 72 |
| 5859 | CCCCGCGCCCGCTGCGCAGG | 71 |
| 5860 | GCCCCGCGCCCGCTGCGCAG | 70 |
| 5861 | TGCCCCGCGCCCGCTGCGCA | 69 |
| 5862 | CTGCCCCGCGCCCGCTGCGC | 68 |
| 5863 | GCTGCCCCGCGCCCGCTGCG | 67 |
| 5864 | CGCTGCCCCGCGCCCGCTGC | 66 |
| 5865 | GCGCTGCCCCGCGCCCGCTG | 65 |
| 5866 | TGCGCTGCCCCGCGCCCGCT | 64 |
| 5867 | CTGCGCTGCCCCGCGCCCGC | 63 |
| 5868 | GCTGCGCTGCCCCGCGCCCG | 62 |
| 5869 | CGCTGCGCTGCCCCGCGCCC | 61 |
| 5870 | CCGCTGCGCTGCCCCGCGCC | 60 |
| 5871 | CCCGCTGCGCTGCCCCGCGC | 59 |
| 5872 | TCCCGCTGCGCTGCCCCGCG | 58 |
| 5873 | CTCCCGCTGCGCTGCCCCGC | 57 |
| 5874 | CCTCCCGCTGCGCTGCCCCG | 56 |
| 5875 | TCCTCCCGCTGCGCTGCCCC | 55 |
| 5876 | TTCCTCCCGCTGCGCTGCCC | 54 |
| 5877 | CTTCCTCCCGCTGCGCTGCC | 53 |
| 5878 | GCTTCCTCCCGCTGCGCTGC | 52 |
| 5879 | CGCTTCCTCCCGCTGCGCTG | 51 |
| 5880 | TCGCTTCCTCCCGCTGCGCT | 50 |
| 5881 | CTCGCTTCCTCCCGCTGCGC | 49 |
| 5882 | TCTCGCTTCCTCCCGCTGCG | 48 |
| 5883 | CTCTCGCTTCCTCCCGCTGC | 47 |
| 5884 | CCTCTCGCTTCCTCCCGCTG | 46 |
| 5885 | ACCTCTCGCTTCCTCCCGCT | 45 |
| 5886 | CACCTCTCGCTTCCTCCCGC | 44 |
| 5887 | GCACCTCTCGCTTCCTCCCG | 43 |
| 5888 | AGCACCTCTCGCTTCCTCCC | 42 |
| 5889 | CAGCACCTCTCGCTTCCTCC | 41 |
| 5890 | GCAGCACCTCTCGCTTCCTC | 40 |
| 5891 | GGCAGCACCTCTCGCTTCCT | 39 |
| 5892 | GGGCAGCACCTCTCGCTTCC | 38 |
| 5893 | AGGGCAGCACCTCTCGCTTC | 37 |
| 5894 | GAGGGCAGCACCTCTCGCTT | 36 |
| 5895 | GGAGGGCAGCACCTCTCGCT | 35 |
| 5896 | GGGAGGGCAGCACCTCTCGC | 34 |
| 5897 | GGGGAGGGCAGCACCTCTCG | 33 |
| 5898 | GCGGAGCGGGCTGAACGTGCG | 249 |
| 5899 | CGGAGCGGGCTGAACGTGCG | 250 |
| 5900 | GACTGGAGGCCGGGGGAGGGGCGGGG | 433 |
| 5901 | GACCCGGGTAACGCGCTTCCAAC | 5 |
| 5902 | ACCCGGGTAACGCGCTTCCA | 6 |
| 5903 | CCCGGGTAACGCGCTTCCAA | 7 |
| 5904 | CCGGGTAACGCGCTTCCAAC | 8 |
| 5905 | CGGGTAACGCGCTTCCAACT | 9 |
| 5906 | GGGTAACGCGCTTCCAACTC | 10 |
| 5907 | GGTAACGCGCTTCCAACTCC | 11 |
| 5908 | GTAACGCGCTTCCAACTCCG | 12 |
| 5909 | TAACGCGCTTCCAACTCCGG | 13 |
| 5910 | AACGCGCTTCCAACTCCGGG | 14 |
| 5911 | ACGCGCTTCCAACTCCGGGG | 15 |
| 5912 | CGCGCTTCCAACTCCGGGGG | 16 |
| 5913 | GCGCTTCCAACTCCGGGGGG | 17 |
| 5914 | CGCTTCCAACTCCGGGGGGA | 18 |
| 5915 | GCTTCCAACTCCGGGGGGAG | 19 |
| 5916 | CTTCCAACTCCGGGGGGAGG | 20 |
| 5917 | TTCCAACTCCGGGGGGAGGG | 21 |
| 5918 | TCCAACTCCGGGGGGAGGGC | 22 |
| 5919 | CCAACTCCGGGGGGAGGGCA | 23 |
| 5920 | GGACCCGGGTAACGCGCTTC | 4 |
| 5921 | TGGACCCGGGTAACGCGCTT | 3 |
| 5922 | TTGGACCCGGGTAACGCGCT | 2 |
| 5923 | TTTGGACCCGGGTAACGCGC | 1 |
| 5924 | CACTCGGCTCCGCCCCTATTGC | 507 |
| 5925 | ACTCGGCTCCGCCCCTATTG | 508 |
| 5926 | CTCGGCTCCGCCCCTATTGC | 509 |
| 5927 | TCGGCTCCGCCCCTATTGCC | 510 |
| 5928 | CGGCTCCGCCCCTATTGCCT | 511 |
| 5929 | GGCTCCGCCCCTATTGCCTC | 512 |
| 5930 | GCTCCGCCCCTATTGCCTCG | 513 |
| 5931 | CTCCGCCCCTATTGCCTCGC | 514 |
| 5932 | TCCGCCCCTATTGCCTCGCA | 515 |
| 5933 | CCGCCCCTATTGCCTCGCAG | 516 |
| 5934 | CGCCCCTATTGCCTCGCAGA | 517 |
| 5935 | GCCCCTATTGCCTCGCAGAC | 518 |
| 5936 | CCCCTATTGCCTCGCAGACA | 519 |
| 5937 | CCCTATTGCCTCGCAGACAA | 520 |
| 5938 | CCTATTGCCTCGCAGACAAC | 521 |
| 5939 | CTATTGCCTCGCAGACAACC | 522 |
| 5940 | TATTGCCTCGCAGACAACCA | 523 |
| 5941 | ATTGCCTCGCAGACAACCAA | 524 |
| 5942 | TTGCCTCGCAGACAACCAAT | 525 |
| 5943 | TGCCTCGCAGACAACCAATG | 526 |
| 5944 | GCCTCGCAGACAACCAATGG | 527 |
| 5945 | CCTCGCAGACAACCAATGGG | 528 |
| 5946 | CTCGCAGACAACCAATGGGG | 529 |
| 5947 | TCGCAGACAACCAATGGGGG | 530 |
| 5948 | CGCAGACAACCAATGGGGGC | 531 |
| 5949 | CCACTCGGCTCCGCCCCTAT | 506 |
| 5950 | CCCACTCGGCTCCGCCCCTA | 505 |
| 5951 | TCCCACTCGGCTCCGCCCCT | 504 |
| 5952 | CTCCCACTCGGCTCCGCCCC | 503 |
| 5953 | ACTCCCACTCGGCTCCGCCC | 502 |
| 5954 | CACTCCCACTCGGCTCCGCC | 501 |
| 5955 | ACACTCCCACTCGGCTCCGC | 500 |
| 5956 | CACACTCCCACTCGGCTCCG | 499 |
| 5957 | CCACACTCCCACTCGGCTCC | 498 |
| 5958 | TCCACACTCCCACTCGGCTC | 497 |
| 5959 | TTCCACACTCCCACTCGGCT | 496 |
| 5960 | TTTCCACACTCCCACTCGGC | 495 |
| 5961 | CTTTCCACACTCCCACTCGG | 494 |
| 5962 | GCTTTCCACACTCCCACTCG | 493 |
| 5963 | CGCTTTCCACACTCCCACTC | 492 |
| 5964 | GCGCTTTCCACACTCCCACT | 491 |
| 5965 | GGCGCTTTCCACACTCCCAC | 490 |
| 5966 | CGGCGCTTTCCACACTCCCA | 489 |
| 5967 | GCGGCGCTTTCCACACTCCC | 488 |
| 5968 | TGCGGCGCTTTCCACACTCC | 487 |
| 5969 | ATGCGGCGCTTTCCACACTC | 486 |
| 5970 | GATGCGGCGCTTTCCACACT | 485 |
| 5971 | GGATGCGGCGCTTTCCACAC | 484 |
| 5972 | GGGATGCGGCGCTTTCCACA | 483 |
| 5973 | CGGGATGCGGCGCTTTCCAC | 482 |
| 5974 | CCGGGATGCGGCGCTTTCCA | 481 |
| 5975 | CCCGGGATGCGGCGCTTTCC | 480 |
| 5976 | ACCCGGGATGCGGCGCTTTC | 479 |
| 5977 | CACCCGGGATGCGGCGCTTT | 478 |
| 5978 | CCACCCGGGATGCGGCGCTT | 477 |
| 5979 | CCCACCCGGGATGCGGCGCT | 476 |
| 5980 | TCCCACCCGGGATGCGGCGC | 475 |
| 5981 | CTCCCACCCGGGATGCGGCG | 474 |
| 5982 | CCTCCCACCCGGGATGCGGC | 473 |
| 5983 | GCCTCCCACCCGGGATGCGG | 472 |
| 5984 | CGCCTCCCACCCGGGATGCG | 471 |
| 5985 | TCGCCTCCCACCCGGGATGC | 470 |
| 5986 | CTCGCCTCCCACCCGGGATG | 469 |
| 5987 | CCTCGCCTCCCACCCGGGAT | 468 |
| 5988 | GCCTCGCCTCCCACCCGGGA | 467 |
| 5989 | AGCCTCGCCTCCCACCCGGG | 466 |
| 5990 | AAGCCTCGCCTCCCACCCGG | 465 |
| 5991 | GAAGCCTCGCCTCCCACCCG | 464 |
| 5992 | GGAAGCCTCGCCTCCCACCC | 463 |
| 5993 | GGGAAGCCTCGCCTCCCACC | 462 |
| 5994 | GGGGAAGCCTCGCCTCCCAC | 461 |
| 5995 | AGGGGAAGCCTCGCCTCCCA | 460 |
| 5996 | AAGGGGAAGCCTCGCCTCCC | 459 |
| 5997 | GAAGGGGAAGCCTCGCCTCC | 458 |
| 5998 | GGAAGGGGAAGCCTCGCCTC | 457 |
| 5999 | GGGAAGGGGAAGCCTCGCCT | 456 |
| 6000 | TACGTCACGGGAGCGCGGCGCAC | 578 |
| 6001 | ACGTCACGGGAGCGCGGCGC | 579 |
| 6002 | CGTCACGGGAGCGCGGCGCA | 580 |
| 6003 | GTCACGGGAGCGCGGCGCAC | 581 |
| 6004 | TCACGGGAGCGCGGCGCACT | 582 |
| 6005 | CACGGGAGCGCGGCGCACTG | 583 |
| 6006 | ACGGGAGCGCGGCGCACTGC | 584 |
| 6007 | CGGGAGCGCGGCGCACTGCC | 585 |
| 6008 | GGGAGCGCGGCGCACTGCCT | 586 |
| 6009 | GGAGCGCGGCGCACTGCCTG | 587 |
| 6010 | GAGCGCGGCGCACTGCCTGG | 588 |
| 6011 | AGCGCGGCGCACTGCCTGGG | 589 |
| 6012 | GCGCGGCGCACTGCCTGGGG | 590 |
| 6013 | CGCGGCGCACTGCCTGGGGG | 591 |
| 6014 | GCGGCGCACTGCCTGGGGGC | 592 |
| 6015 | CGGCGCACTGCCTGGGGGCG | 593 |
| 6016 | GGCGCACTGCCTGGGGGCGG | 594 |
| 6017 | GCGCACTGCCTGGGGGCGGG | 595 |
| 6018 | CGCACTGCCTGGGGGCGGGG | 596 |
| 6019 | GCACTGCCTGGGGGCGGGGT | 597 |
| 6020 | CACTGCCTGGGGGCGGGGTC | 598 |
| 6021 | ACTGCCTGGGGGCGGGGTCC | 599 |
| 6022 | CTGCCTGGGGGCGGGGTCCG | 600 |
| 6023 | TGCCTGGGGGCGGGGTCCGT | 601 |
| 6024 | GCCTGGGGGCGGGGTCCGTC | 602 |
| 6025 | CCTGGGGGCGGGGTCCGTCG | 603 |
| 6026 | CTGGGGGCGGGGTCCGTCGC | 604 |
| 6027 | TGGGGGCGGGGTCCGTCGCG | 605 |
| 6028 | GGGGGCGGGGTCCGTCGCGG | 606 |
| 6029 | GGGGCGGGGTCCGTCGCGGA | 607 |
| 6030 | GGGCGGGGTCCGTCGCGGAC | 608 |
| 6031 | GGCGGGGTCCGTCGCGGACG | 609 |
| 6032 | GCGGGGTCCGTCGCGGACGC | 610 |
| 6033 | CGGGGTCCGTCGCGGACGCC | 611 |
| 6034 | GGGGTCCGTCGCGGACGCCG | 612 |
| 6035 | GGGTCCGTCGCGGACGCCGT | 613 |
| 6036 | GGTCCGTCGCGGACGCCGTG | 614 |
| 6037 | GTCCGTCGCGGACGCCGTGG | 615 |
| 6038 | TCCGTCGCGGACGCCGTGGC | 616 |
| 6039 | CCGTCGCGGACGCCGTGGCG | 617 |
| 6040 | CGTCGCGGACGCCGTGGCGC | 618 |
| 6041 | GTCGCGGACGCCGTGGCGCC | 619 |
| 6042 | TCGCGGACGCCGTGGCGCCC | 620 |
| 6043 | CGCGGACGCCGTGGCGCCCT | 621 |
| 6044 | GCGGACGCCGTGGCGCCCTC | 622 |
| 6045 | CGGACGCCGTGGCGCCCTCT | 623 |
| 6046 | GGACGCCGTGGCGCCCTCTG | 624 |
| 6047 | GACGCCGTGGCGCCCTCTGT | 625 |
| 6048 | ACGCCGTGGCGCCCTCTGTC | 626 |
| 6049 | CGCCGTGGCGCCCTCTGTCG | 627 |
| 6050 | GCCGTGGCGCCCTCTGTCGC | 628 |
| 6051 | CCGTGGCGCCCTCTGTCGCC | 629 |
| 6052 | CGTGGCGCCCTCTGTCGCCC | 630 |
| 6053 | GTGGCGCCCTCTGTCGCCCC | 631 |
| 6054 | TGGCGCCCTCTGTCGCCCCG | 632 |
| 6055 | GGCGCCCTCTGTCGCCCCGA | 633 |
| 6056 | GCGCCCTCTGTCGCCCCGAG | 634 |
| 6057 | CGCCCTCTGTCGCCCCGAGG | 635 |
| 6058 | GCCCTCTGTCGCCCCGAGGC | 636 |
| 6059 | CCCTCTGTCGCCCCGAGGCA | 637 |
| 6060 | CCTCTGTCGCCCCGAGGCAA | 638 |
| 6061 | CTCTGTCGCCCCGAGGCAAG | 639 |
| 6062 | TCTGTCGCCCCGAGGCAAGC | 640 |
| 6063 | CTGTCGCCCCGAGGCAAGCA | 641 |
| 6064 | TGTCGCCCCGAGGCAAGCAG | 642 |
| 6065 | GTCGCCCCGAGGCAAGCAGG | 643 |
| 6066 | TCGCCCCGAGGCAAGCAGGT | 644 |
| 6067 | CGCCCCGAGGCAAGCAGGTG | 645 |
| 6068 | GCCCCGAGGCAAGCAGGTGG | 646 |
| 6069 | CCCCGAGGCAAGCAGGTGGA | 647 |
| 6070 | CCCGAGGCAAGCAGGTGGAC | 648 |
| 6071 | CCGAGGCAAGCAGGTGGACC | 649 |
| 6072 | CGAGGCAAGCAGGTGGACCC | 650 |
| 6073 | ATACGTCACGGGAGCGCGGC | 577 |
| 6074 | AATACGTCACGGGAGCGCGG | 576 |
| 6075 | AAATACGTCACGGGAGCGCG | 575 |
| 6076 | GAAATACGTCACGGGAGCGC | 574 |
| 6077 | GTCGCGGACGCCGTGGCGCCCTCTGTC | 619 |
| 6078 | TCGCGGACGCCGTGGCGCCC | 620 |
| 6079 | CGCGGACGCCGTGGCGCCCT | 621 |
| 6080 | GCGGACGCCGTGGCGCCCTC | 622 |
| 6081 | CGGACGCCGTGGCGCCCTCT | 623 |
| 6082 | GGACGCCGTGGCGCCCTCTG | 624 |
| 6083 | GACGCCGTGGCGCCCTCTGT | 625 |
| 6084 | ACGCCGTGGCGCCCTCTGTC | 626 |
| 6085 | CGCCGTGGCGCCCTCTGTCG | 627 |
| 6086 | GCCGTGGCGCCCTCTGTCGC | 628 |
| 6087 | CCGTGGCGCCCTCTGTCGCC | 629 |
| 6088 | CGTGGCGCCCTCTGTCGCCC | 630 |
| 6089 | GTGGCGCCCTCTGTCGCCCC | 631 |
| 6090 | TGGCGCCCTCTGTCGCCCCG | 632 |
| 6091 | GGCGCCCTCTGTCGCCCCGA | 633 |
| 6092 | GCGCCCTCTGTCGCCCCGAG | 634 |
| 6093 | CGCCCTCTGTCGCCCCGAGG | 635 |
| 6094 | GCCCTCTGTCGCCCCGAGGC | 636 |
| 6095 | CCCTCTGTCGCCCCGAGGCA | 637 |
| 6096 | CCTCTGTCGCCCCGAGGCAA | 638 |
| 6097 | CTCTGTCGCCCCGAGGCAAG | 639 |
| 6098 | TCTGTCGCCCCGAGGCAAGC | 640 |
| 6099 | CTGTCGCCCCGAGGCAAGCA | 641 |
| 6100 | TGTCGCCCCGAGGCAAGCAG | 642 |
| 6101 | GTCGCCCCGAGGCAAGCAGG | 643 |
| 6102 | TCGCCCCGAGGCAAGCAGGT | 644 |
| 6103 | CGCCCCGAGGCAAGCAGGTG | 645 |
| 6104 | GCCCCGAGGCAAGCAGGTGG | 646 |
| 6105 | CCCCGAGGCAAGCAGGTGGA | 647 |
| 6106 | CCCGAGGCAAGCAGGTGGAC | 648 |
| 6107 | CCGAGGCAAGCAGGTGGACC | 649 |
| 6108 | CGAGGCAAGCAGGTGGACCC | 650 |
| 6109 | CGTCGCGGACGCCGTGGCGC | 618 |
| 6110 | CCGTCGCGGACGCCGTGGCG | 617 |
| 6111 | TCCGTCGCGGACGCCGTGGC | 616 |
| 6112 | GTCCGTCGCGGACGCCGTGG | 615 |
| 6113 | GGTCCGTCGCGGACGCCGTG | 614 |
| 6114 | GGGTCCGTCGCGGACGCCGT | 613 |
| 6115 | GGGGTCCGTCGCGGACGCCG | 612 |
| 6116 | CGGGGTCCGTCGCGGACGCC | 611 |
| 6117 | GCGGGGTCCGTCGCGGACGC | 610 |
| 6118 | GGCGGGGTCCGTCGCGGACG | 609 |
| 6119 | GGGCGGGGTCCGTCGCGGAC | 608 |
| 6120 | GGGGCGGGGTCCGTCGCGGA | 607 |
| 6121 | GGGGGCGGGGTCCGTCGCGG | 606 |
| 6122 | TGGGGGCGGGGTCCGTCGCG | 605 |
| 6123 | CTGGGGGCGGGGTCCGTCGC | 604 |
| 6124 | CCTGGGGGCGGGGTCCGTCG | 603 |
| 6125 | GCCTGGGGGCGGGGTCCGTC | 602 |
| 6126 | TGCCTGGGGGCGGGGTCCGT | 601 |
| 6127 | CTGCCTGGGGGCGGGGTCCG | 600 |
| 6128 | ACTGCCTGGGGGCGGGGTCC | 599 |
| 6129 | CACTGCCTGGGGGCGGGGTC | 598 |
| 6130 | GCACTGCCTGGGGGCGGGGT | 597 |
| 6131 | CGCACTGCCTGGGGGCGGGG | 596 |
| 6132 | GCGCACTGCCTGGGGGCGGG | 595 |
| 6133 | GGCGCACTGCCTGGGGGCGG | 594 |
| 6134 | CGGCGCACTGCCTGGGGGCG | 593 |
| 6135 | GCGGCGCACTGCCTGGGGGC | 592 |
| 6136 | CGCGGCGCACTGCCTGGGGG | 591 |
| 6137 | GCGCGGCGCACTGCCTGGGG | 590 |
| 6138 | AGCGCGGCGCACTGCCTGGG | 589 |
| 6139 | GAGCGCGGCGCACTGCCTGG | 588 |
| 6140 | GGAGCGCGGCGCACTGCCTG | 587 |
| 6141 | GGGAGCGCGGCGCACTGCCT | 586 |
| 6142 | CGGGAGCGCGGCGCACTGCC | 585 |
| 6143 | ACGGGAGCGCGGCGCACTGC | 584 |
| 6144 | CACGGGAGCGCGGCGCACTG | 583 |
| 6145 | TCACGGGAGCGCGGCGCACT | 582 |
| 6146 | GTCACGGGAGCGCGGCGCAC | 581 |
| 6147 | CGTCACGGGAGCGCGGCGCA | 580 |
| 6148 | ACGTCACGGGAGCGCGGCGC | 579 |
| 6149 | TACGTCACGGGAGCGCGGCG | 578 |
| 6150 | ATACGTCACGGGAGCGCGGC | 577 |
| 6151 | AATACGTCACGGGAGCGCGG | 576 |
| 6152 | AAATACGTCACGGGAGCGCG | 575 |
| 6153 | GAAATACGTCACGGGAGCGC | 574 |
| 6154 | CACTCGCCGTCATGCCCGGATCC | 1183 |
| 6155 | ACTCGCCGTCATGCCCGGAT | 1184 |
| 6156 | CTCGCCGTCATGCCCGGATC | 1185 |
| 6157 | TCGCCGTCATGCCCGGATCC | 1186 |
| 6158 | CGCCGTCATGCCCGGATCCT | 1187 |
| 6159 | GCCGTCATGCCCGGATCCTT | 1188 |
| 6160 | CCGTCATGCCCGGATCCTTT | 1189 |
| 6161 | CGTCATGCCCGGATCCTTTT | 1190 |
| 6162 | GTCATGCCCGGATCCTTTTT | 1191 |
| 6163 | TCATGCCCGGATCCTTTTTG | 1192 |
| 6164 | CATGCCCGGATCCTTTTTGT | 1193 |
| 6165 | ATGCCCGGATCCTTTTTGTA | 1194 |
| 6166 | TGCCCGGATCCTTTTTGTAT | 1195 |
| 6167 | GCCCGGATCCTTTTTGTATT | 1196 |
| 6168 | GCACTCGCCGTCATGCCCGG | 1182 |
| 6169 | GGCACTCGCCGTCATGCCCG | 1181 |
| 6170 | AGGCACTCGCCGTCATGCCC | 1180 |
| 6171 | CAGGCACTCGCCGTCATGCC | 1179 |
| 6172 | ACAGGCACTCGCCGTCATGC | 1178 |
| 6173 | TACAGGCACTCGCCGTCATG | 1177 |
| 6174 | TTACAGGCACTCGCCGTCAT | 1176 |
| 6175 | ATTACAGGCACTCGCCGTCA | 1175 |
| 6176 | GATTACAGGCACTCGCCGTC | 1174 |
| 6177 | GGATTACAGGCACTCGCCGT | 1173 |
| 6178 | GGGATTACAGGCACTCGCCG | 1172 |
| 6179 | TGGGATTACAGGCACTCGCC | 1171 |
| 6180 | CTGGGATTACAGGCACTCGC | 1170 |
| 6181 | GCTGGGATTACAGGCACTCG | 1169 |

Hot Zones (Relative upstream location to gene start site)
1-950
1050-1500

### Examples

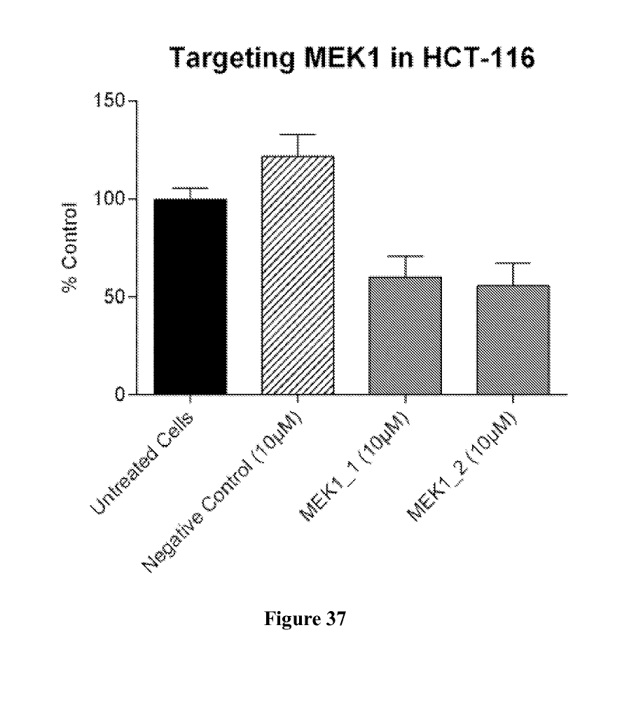

In FIG. 37, In HCT-116 (human colorectal carcinoma), MEK1_{—}1 (216) and MEK1_{—}2 (212) produced statistically significant (P<0.05) inhibition at 10 μM compared to the untreated and negative control values. The MEK1 sequences MEK1_{—}1 (216) and MEK1_{—}2 (212) fit the independent and dependent DNAi motif claims.

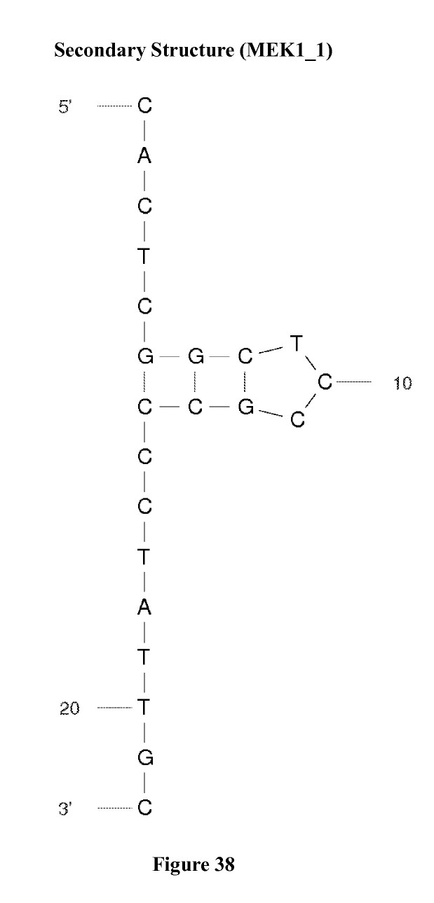

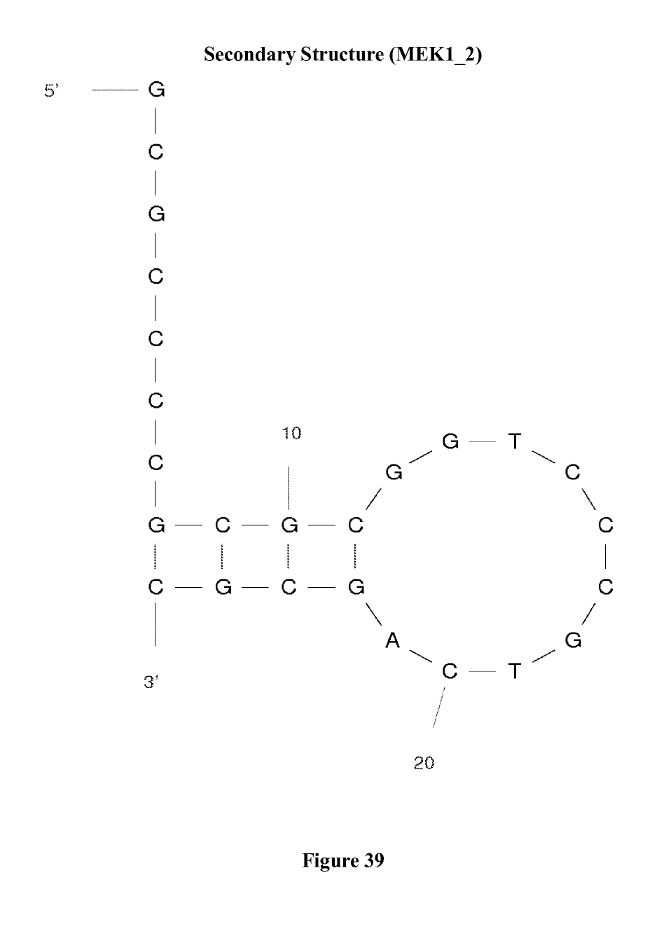

The secondary structures for MEK1_{—}1 (216) and MEK1_{—}2 (212) are shown in FIGS. 38 and 39.

Genetic Code (5′ Upstream Region)

(SEQ ID NO: 11969)

ACATATAGTTCAGTCTTATTCTTGTCTGTATGGTCAGCACTTATGTTAGG
CCCTCAGGAAAAGTTGACAGAACCGATGGATCACTGCCGGTCTGAAAAGG
AAATGAGGAAAACAAATTCTCCTACCTTGAACTATTCTGCAAACTTTAAC
CATTGGGGTAATTGTTTATCTGGGCTTCTTGGATCATGATAAGGGCTTAG
GGTTTACTCAGTGGAGGCCAACCCAGCATGCATAGAATCATAATATTTCA
ATATTAAAAAGAATGCTGCATTTTACACAGAGTGGAAGTGAGGCCTTGAA
AATTTCAATTAATTGCTCAAAGTCCTAATAGTTTTTATTTGAACTAGTAA
ATATAAAATTATACCAGAATTCAGATAGACTGCCTTGATAATAGATTACT
TTGAAAAGTTTCAATTTTTTTTTTTTTTTTGAGATAGTCTCACTGTGTTG
CACAGGCTGGAGTACAGTGGAGTGATCTTGGCTCACTGTAACCTCCACCT
CCTGGGTTCAAGTGATTCTCCAGCTTCAGCCTCCCAAGTAGCTGGGACTA
CAGGCACCCGCCACCACATTCAGATAATTTTTGTATTTTTAGTAAAGACA
GGGTTTCACCATGTTGGCCAGGCTTGGTCTTGAACTCCTGACCTCAGGTG
ATCCTCCCACCTCAGCCTCCCAAAGTGCTGGGATTAAAGGTGTGAGCCAC
CACCACACCTGGCCTTCAATTCACTTTTTAATGTTTATTATTTTACTCTG
ATACTAAAAATTATGCATGTTTAACATGAATAAGGACACACTTCTACACA
CACATGCATACATTTACATCTATGCCTCTATATTAAAAAGTATGGGGGAA
AGAAATGGGGAGATGTAGGTCAAAGAATATAAAGCAGCAGATATGTAGGA
TGAAGAAGTCTAGAGATCTAATGTACAACATGAAGACCATAGTTAATAAC
ATTGTATTTTATTTGCGTTTTTTGTTAAATAAGTAGATTTTAGCTGCTCG
TCATACTTTACACAAGCCTTTATGTGACGGTATAGATATGTTAATTCACT
TCACTATAGTAACCATTTTACTATCTATATATATCCCATAACATCATGTT
ACAAACCTCAAATATACACAATAAAATTTATTTTTATTTATTTAATTTAT
TTATTTATTTTTGAGACGGAGTCTTGTTCTGTCGCCCAGGCTGGAGTGCA
GTGGCGCGATCTCGGCTCACTGCAAGCTCCACCTCCCGGGTTCACACCAT
TCTCCTGCCTCAGCCTCCTGAGTAGCTGGGACTACAGGCACCCACCACCA
CGCCCGGCTAATTTTTTGTATTTTTTAGTAGAGATGGGGTTTCACCGTGT
TAGCCAGGATGGTCTCGATTTCCTGACCTCGTGATCTGCCCACCTCAGCC
TCCCAAAGTGCTGGGATTACAGGCATGAGCCACCGCGCCCGGCCTATTTT
ATTTATTTTTGAGACAGAGTCTTGCTCTGTTGCCCAGGCTGGAGTGCAGT
GGTGCAATCTCGGCTCACTGCAAACTCTGCCTCCCTGGTTCAGGCAATTA
TCCTGCCTCAGCCTCCTGAGTAGCTGGGATTACAGGTGCCCACCACCATG
CCTGGCTAATTTTTGTAATTTAGTAGAGACGGGGTTTCACCATGTTGGCC
AGGCTGATCTTGAAGTCCTGACCTCAAGTGATCTTCCAGCTTTGGCCTCA
CAAAGTGCTGGGATTACAGGTGGTAGCCGCCACTGCATCCACCCAGAATA
ATTTATTTTTTAAAAAACTATGAGTTCAGGCCGGGCGCAGTGGCTCACGC
CTGTAAACCCAGCACTTTGGGAGGCCGAGGTGGGCGGATCACCTGAGGTC
AGGAGTTTGAGACCAGCCTGGCCAACATGGTGAAATCCTGTCTCTACTAA
AAATACAAAATTAGCCAGGCATGGTGGTGCATGCCTGTAATCCCAGCTAC
TTGGGAGGCTGAGGCAGGAGAATCACTTGAGCTTGGGAGGTGGAGGTTGC
AATGAGCCAAGGTTGCGCCATTGCACTCAAGCCTGGGCAAAAAGAGCAAA
ACGCCACTCAAAAACAAAAACAAAACAAAACAAAAACACCCCCCCAAAAA
ACAAAACAAAACAATGAGTTCACACTGATACCTCCAATTCCAATACAATA
GCGTAAGGTATTCTCCCTTCCCATACTTCTAACGTCATTCTACCACAGTG
AGAAAGCTGGCTCTGTCATGCTTAATATATTTAGTGACTTAATCAACCAT
CCTGAATGCAACTAACCTCCCATCTAAGCTTCTAGGCCTTCCCCACTTGG
ATGCCTTGTTCTCCCCTCTTGGGCCCTACGGCTAAGACTTTGTGTAGGAC
TGCCTCCCAGGTGTTCAAGCCCTCTTCATTTTCTCAGGTTCCTCAGCCTC
CTTACCTGCTAGGTCACCAACACCTGGCTGTGGATAACCAGGTGTAGATG
TTTCCTTTGTTCTGTACACGTTTCCTTTGTTCTGTACACCTAATGTCTTT
GACACTTAGTATTTTAGGATGGGAAAGGGGAAGAGGAACACTGAATGTGC
ACTTTTAAATGGGTATTGTGCCTCTTATTAAGCTCTTTATTCACATCTTA
TTTCTTTAGTAATTCACAGAATTGGAATTTTTGGATTAAAGTTCTTTTTT
TTTTTGAGACGGGGTCTCACTCTGTCGCCCAGGCTGGAGTGCAGTGGTGT
GATCTTGGATCACTGCAACCTCCGCCTCCCGAGTTCAAGCAATTCTCTGC
CTCAGCCCCCCAAGTAGTTGGGATTACAGGCACCCGCCACCACGCCCAGC
TAATTTTTTGTATTTTTAGTAGAGATGGGTTTCACCATCTTGGCCAGGCT
GGTCTTGAACTCCTGACCTCGTGATCCACCCGTCTCGGCCTCCCAAAGTT
CTGGAATTACAGGCGTGAGCCACCGCGCCTGGCCTGGATGAAAGTTTTTT
TAAAGGGAGTCTTGCTCTGTAGCCCTGGCTGGTGTGCAGTGGTGTGATCA
TAGCTCACTGCAGCCTCAAACTCCTGGGCTCAAGTGATCCTCCAGCCTCA
GCCTCCTCAGTAGCTTGGACGACAGCTGCACACAACCATGCCCAGCTAAT
AGAGACGGGGGACTCACTATGTTGCCCAGGCTAGTCTCGAACTCCTGGGC
TCAAGTGATCCTCTTGCCTGGGCCTCCCAAAATTGGGATTACAGGCGTGA
GCCACCGCTCCTGGCCCGAAAGAGTGTTTTTAAGGCTTTAAAAAAATATT
GCCAACATGGTGAAAACCCGTTTCTACAAAAATACAAAAAGGATCCGGGC
ATGACGGCGAGTGCCTGTAATCCCAGCTACTCAGGAGACTGAGGCAGGAG
AATCGCTTGAACGTGGGAGGCAGAGGTGGTAGTTAGCGGAGATCGCGCCA
CTACACTCCAGGCTGGGCAACTGAGGGAGACACCGTCTTAAAAAAAAAAA
AGTTCCCAAGTCTAAAAAAAAAAAAATCATCAATCTGCTCTCAAAAACTG
TCGCAACAATTTACAATCTCATCAGCACTGAGTATCCATTTCCTTGCACC
CTTCTCAGTAGTATTACCATTAAACAAACAAAATTTATATGCGTCAGTTT
GTTGGGCTCAAAGGAGCCTCTCGACAAGTTTCCTATTCCCCACGCTGCCT
CTCCTCTGGACACAGGAAGGGGTCCTTTTCCTTATTTATTTTGTTATTTC
ATTTTCGTCAACACGGCTCGGCTTGGGGACAGGGGTCGGGGGCAGGCCGG
TTACCGCAGAGGTGGAGGCCGCGCGGCACCTGGCCTGGAGAGCTCACCAC
ACAGCGACACAGACTTCTTCTCAGCTGGGTCCACCTGCTTGCCTCGGGGC
GACAGAGGGCGCCACGGCGTCCGCGACGGACCCCGCCCCCAGGCAGTGCG
CCGCGCTCCCGTGACGTATTTCCGCGTCATCTGCCGCCGAGGCTTGCCCC
CATTGGTTGTCTGCGAGGCAATAGGGGCGGAGCCGAGTGGGAGTGTGGAA
AGCGCCGCATCCCGGGTGGGAGGCGAGGCTTCCCCTTCCCCGCCCCTCCC
CCGGCCTCCAGTCCCTCCCAGGGCCGCTTCGCAGAGCGGCTAGGAGCACG
GCGGCGGCGGCACTTTCCCCGGCAGGAGCTGGAGCTGGGCTCTGGTGCGC
GCGCGGCTGTGCCGCCCGAGCCGGAGGGACTGGTTGGTTGAGAGAGAGAG
AGGAAGGGAATCCCGGGCTGCCGAACCGCACGTTCAGCCCGCTCCGCTCC
TGCAGGGCAGCCTTTCGGCTCTCTGCGCGCGAAGCCGAGTCCCGGGCGGG
TGGGGCGGGGGTCCACTGAGACCGCTACCGGCCCCTCGGCGCTGACGGGA
CCGCGCGGGGCGCACCCGCTGAAGGCAGCCCCGGGGCCCGCGGCCCGGAC
TTGGTCCTGCGCAGCGGGCGCGGGGCAGCGCAGCGGGAGGAAGCGAGAGG
TGCTGCCCTCCCCCCGGAGTTGGAAGCGCGTTACCCGGGTCCAAAATG

21) MEK1 and MEK2 (MAP2K2) Mitogen-activated protein kinase kinase 1. Dual specificity protein kinases act as an essential component of the MAP kinase signal transduction pathway and serves as an integration point for multiple biochemical signals. MEK1 and MEK2 are members of the dual specificity protein kinase family, which act as a mitogen-activated protein (MAP) kinase kinases and as extracellular signal-regulated kinases (ERKs). Binding of extracellular ligands such as growth factors, cytokines and hormones to their cell-surface receptors activates RAS and this initiates RAF1 activation. RAF1 then further activates the dual-specificity protein kinases MAP2K1/MEK1 and MAP2K2/MEK2. Both MAP2K1/MEK1 and MAP2K2/MEK2 function specifically in the MAPK/ERK cascade, and catalyze the concomitant phosphorylation of a threonine and a tyrosine residue in a Thr-Glu-Tyr sequence located in the extracellular signal-regulated kinases MAPK3/ERK1 and MAPK1/ERK2, leading to their activation and further transduction of the signal within the MAPK/ERK cascade. Depending on the cellular context, this pathway mediates diverse biological functions such as cell growth and proliferation, adhesion, survival and differentiation, predominantly through the regulation of transcription, metabolism and cytoskeletal rearrangements (reviewed by Roberts and Der; 2007 Oncogene 26, 3291-3310).

Genetic alterations that activate the mitogen-activated protein kinase (MAP kinase) pathway occur commonly in cancer. For example, the majority of melanomas harbor mutations in the BRAF oncogene, which confers enhanced sensitivity to pharmacologic MAP kinase inhibition (e.g., RAF or MEK inhibitors). Most mutations conferring resistance to MEK inhibition in vitro populated the allosteric drug binding pocket or alpha-helix C and showed robust (approximately 100-fold) resistance to allosteric MEK inhibition (reviewed in Emery et al, 2009; Proc Natl Acad Sci.; 106(48):20411-20416). Other mutations affected MEK1 codons located within or abutting the N-terminal negative regulatory helix (helix A), which also undergo gain-of-function germline mutations in cardiofaciocutaneous (CFC) syndrome. One target of the MAPK/ERK cascade is peroxisome proliferator-activated receptor gamma (PPARG), a nuclear receptor that promotes differentiation and apoptosis. MAP2K1/MEK1 has been shown to export PPARG from the nucleus. The MAPK/ERK cascade is also involved in the regulation of endosomal dynamics, including lysosome processing and endosome cycling through the perinuclear recycling compartment (PNRC), as well as in the fragmentation of the Golgi apparatus during mitosis.

Protein: MEK2 Gene: MAP2K2 (Homo sapiens, chromosome 19, 4090319-4124126 [NCBI Reference Sequence: NC_{—}000019.9]; start site location: 4123872; strand: negative)

Gene Identification

| | |
|---|---|
| GeneID | 5605 |
| HGNC | 6842 |
| HPRD | 03164 |
| MIM | 601263 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 6182 | | CGCCGCAGCCCGAGTCCGAGAGG | 226 |
| 6202 | | GAGGGGCGCTGGGGCTGAGGCGAGCG | 165 |
| 6203 | | CTCGCGATAACGGGATCGGGAGCCGCG | 290 |
| 6235 | MEK2_1 | CCGACGCGAGGCGGTGCCGGGACCGG | 391 |
| 6240 | | CACGGCGCGTGTGCCCAAGCGC | 436 |
| 6299 | | CGTGGACACACGCCCCTAGCCC | 643 |
| 6341 | | TAGACACTTCGGTGAATCGTGCCGC | 1622 |

Target Shift Sequences

| | | Relative |
|---|---|---|
| | | upstream |
| | | location |
| Sequence | | to gene |
| ID No: | Sequence (5′-3′) | start site |
| 6182 | CGCCGCAGCCCGAGTCCGAGAGG | 226 |
| 6183 | GCCGCAGCCCGAGTCCGAGA | 227 |
| 6184 | CCGCAGCCCGAGTCCGAGAG | 228 |
| 6185 | CGCAGCCCGAGTCCGAGAGG | 229 |
| 6186 | GCAGCCCGAGTCCGAGAGGC | 230 |
| 6187 | CAGCCCGAGTCCGAGAGGCA | 231 |
| 6188 | AGCCCGAGTCCGAGAGGCAG | 232 |
| 6189 | GCCCGAGTCCGAGAGGCAGG | 233 |
| 6190 | CCCGAGTCCGAGAGGCAGGG | 234 |
| 6191 | ACGCCGCAGCCCGAGTCCGA | 225 |
| 6192 | GACGCCGCAGCCCGAGTCCG | 224 |
| 6193 | TGACGCCGCAGCCCGAGTCC | 223 |
| 6194 | CTGACGCCGCAGCCCGAGTC | 222 |
| 6195 | GCTGACGCCGCAGCCCGAGT | 221 |
| 6196 | GGCTGACGCCGCAGCCCGAG | 220 |
| 6197 | AGGCTGACGCCGCAGCCCGA | 219 |
| 6198 | AAGGCTGACGCCGCAGCCCG | 218 |
| 6199 | GAAGGCTGACGCCGCAGCCC | 217 |
| 6200 | AGAAGGCTGACGCCGCAGCC | 216 |
| 6201 | AAGAAGGCTGACGCCGCAGC | 215 |
| 6202 | GAGGGGCGCTGGGGCTGAGGCGAGCG | 165 |
| 6203 | CTCGCGATAACGGGATCGGGAGCCGCG | 291 |
| 6204 | TCGCGATAACGGGATCGGGA | 292 |
| 6205 | TCTCGCGATAACGGGATCGG | 290 |
| 6206 | TTCTCGCGATAACGGGATCG | 289 |
| 6207 | CTTCTCGCGATAACGGGATC | 288 |
| 6208 | GCTTCTCGCGATAACGGGAT | 287 |
| 6209 | GGCTTCTCGCGATAACGGGA | 286 |
| 6210 | CGGCTTCTCGCGATAACGGG | 285 |
| 6211 | CCGGCTTCTCGCGATAACGG | 284 |
| 6212 | ACCGGCTTCTCGCGATAACG | 283 |
| 6213 | GACCGGCTTCTCGCGATAAC | 282 |
| 6214 | GGACCGGCTTCTCGCGATAA | 281 |
| 6215 | CGGACCGGCTTCTCGCGATA | 280 |
| 6216 | GCGGACCGGCTTCTCGCGAT | 279 |
| 6217 | CGCGGACCGGCTTCTCGCGA | 278 |
| 6218 | TCGCGGACCGGCTTCTCGCG | 277 |
| 6219 | ATCGCGGACCGGCTTCTCGC | 276 |
| 6220 | GATCGCGGACCGGCTTCTCG | 275 |
| 6221 | AGATCGCGGACCGGCTTCTC | 274 |
| 6222 | AAGATCGCGGACCGGCTTCT | 273 |
| 6223 | CAAGATCGCGGACCGGCTTC | 272 |
| 6224 | ACAAGATCGCGGACCGGCTT | 271 |
| 6225 | CACAAGATCGCGGACCGGCT | 270 |
| 6226 | CCACAAGATCGCGGACCGGC | 269 |
| 6227 | GCCACAAGATCGCGGACCGG | 268 |
| 6228 | GGCCACAAGATCGCGGACCG | 267 |
| 6229 | CGGCCACAAGATCGCGGACC | 266 |
| 6230 | GCGGCCACAAGATCGCGGAC | 265 |
| 6231 | GGCGGCCACAAGATCGCGGA | 264 |
| 6232 | GGGCGGCCACAAGATCGCGG | 263 |
| 6233 | GGGGCGGCCACAAGATCGCG | 262 |
| 6234 | AGGGGCGGCCACAAGATCGC | 261 |
| 6235 | CCGACGCGAGGCGGTGCCGGGACCGG | 391 |
| 6236 | CGACGCGAGGCGGTGCCGGG | 392 |
| 6237 | ACCGACGCGAGGCGGTGCCG | 390 |
| 6238 | GACCGACGCGAGGCGGTGCC | 389 |
| 6239 | AGACCGACGCGAGGCGGTGC | 388 |
| 6240 | CACGGCGCGTGTGCCCAAGCGC | 436 |
| 6241 | ACGGCGCGTGTGCCCAAGCG | 437 |
| 6242 | CGGCGCGTGTGCCCAAGCGC | 438 |
| 6243 | GGCGCGTGTGCCCAAGCGCT | 439 |
| 6244 | GCGCGTGTGCCCAAGCGCTT | 440 |
| 6245 | CGCGTGTGCCCAAGCGCTTG | 441 |
| 6246 | GCGTGTGCCCAAGCGCTTGG | 442 |
| 6247 | CGTGTGCCCAAGCGCTTGGG | 443 |
| 6248 | GTGTGCCCAAGCGCTTGGGG | 444 |
| 6249 | TGTGCCCAAGCGCTTGGGGC | 445 |
| 6250 | GTGCCCAAGCGCTTGGGGCA | 446 |
| 6251 | TGCCCAAGCGCTTGGGGCAT | 447 |
| 6252 | GCCCAAGCGCTTGGGGCATG | 448 |
| 6253 | CCCAAGCGCTTGGGGCATGA | 449 |
| 6254 | CCAAGCGCTTGGGGCATGAG | 450 |
| 6255 | CAAGCGCTTGGGGCATGAGG | 451 |
| 6256 | AAGCGCTTGGGGCATGAGGC | 452 |
| 6257 | AGCGCTTGGGGCATGAGGCG | 453 |
| 6258 | GCGCTTGGGGCATGAGGCGC | 454 |
| 6259 | CGCTTGGGGCATGAGGCGCG | 455 |
| 6260 | GCTTGGGGCATGAGGCGCGG | 456 |
| 6261 | CTTGGGGCATGAGGCGCGGG | 457 |
| 6262 | CCACGGCGCGTGTGCCCAAG | 435 |
| 6263 | ACCACGGCGCGTGTGCCCAA | 434 |
| 6264 | TACCACGGCGCGTGTGCCCA | 433 |
| 6265 | TTACCACGGCGCGTGTGCCC | 432 |
| 6266 | CTTACCACGGCGCGTGTGCC | 431 |
| 6267 | CCTTACCACGGCGCGTGTGC | 430 |
| 6268 | GCCTTACCACGGCGCGTGTG | 429 |
| 6269 | TGCCTTACCACGGCGCGTGT | 428 |
| 6270 | TTGCCTTACCACGGCGCGTG | 427 |
| 6271 | CTTGCCTTACCACGGCGCGT | 426 |
| 6272 | GCTTGCCTTACCACGGCGCG | 425 |
| 6273 | CGCTTGCCTTACCACGGCGC | 424 |
| 6274 | TCGCTTGCCTTACCACGGCG | 423 |
| 6275 | CTCGCTTGCCTTACCACGGC | 422 |
| 6276 | CCTCGCTTGCCTTACCACGG | 421 |
| 6277 | CCCTCGCTTGCCTTACCACG | 420 |
| 6278 | GCCCTCGCTTGCCTTACCAC | 419 |
| 6279 | CGCCCTCGCTTGCCTTACCA | 418 |
| 6280 | GCGCCCTCGCTTGCCTTACC | 417 |
| 6281 | GGCGCCCTCGCTTGCCTTAC | 416 |
| 6282 | GGGCGCCCTCGCTTGCCTTA | 415 |
| 6283 | CGGGCGCCCTCGCTTGCCTT | 414 |
| 6284 | CCGGGCGCCCTCGCTTGCCT | 413 |
| 6285 | ACCGGGCGCCCTCGCTTGCC | 412 |
| 6286 | GACCGGGCGCCCTCGCTTGC | 411 |
| 6287 | GGACCGGGCGCCCTCGCTTG | 410 |
| 6288 | GGGACCGGGCGCCCTCGCTT | 409 |
| 6289 | CGGGACCGGGCGCCCTCGCT | 408 |
| 6290 | CCGGGACCGGGCGCCCTCGC | 407 |
| 6291 | GCCGGGACCGGGCGCCCTCG | 406 |
| 6292 | TGCCGGGACCGGGCGCCCTC | 405 |
| 6293 | GTGCCGGGACCGGGCGCCCT | 404 |
| 6294 | GGTGCCGGGACCGGGCGCCC | 403 |
| 6295 | CGGTGCCGGGACCGGGCGCC | 402 |
| 6296 | GCGGTGCCGGGACCGGGCGC | 401 |
| 6297 | GGCGGTGCCGGGACCGGGCG | 400 |
| 6298 | AGGCGGTGCCGGGACCGGGC | 399 |
| 6299 | CGTGGACACACGCCCCTAGCCC | 648 |
| 6300 | GTGGACACACGCCCCTAGCC | 649 |
| 6301 | TGGACACACGCCCCTAGCCC | 650 |
| 6302 | GGACACACGCCCCTAGCCCC | 651 |
| 6303 | GACACACGCCCCTAGCCCCC | 652 |
| 6304 | ACACACGCCCCTAGCCCCCA | 653 |
| 6305 | CACACGCCCCTAGCCCCCAC | 654 |
| 6306 | ACACGCCCCTAGCCCCCACC | 655 |
| 6307 | CACGCCCCTAGCCCCCACCG | 656 |
| 6308 | ACGCCCCTAGCCCCCACCGC | 657 |
| 6309 | CGCCCCTAGCCCCCACCGCC | 658 |
| 6310 | GCCCCTAGCCCCCACCGCCT | 659 |
| 6311 | CCCCTAGCCCCCACCGCCTT | 660 |
| 6312 | CCCTAGCCCCCACCGCCTTA | 661 |
| 6313 | CCTAGCCCCCACCGCCTTAG | 662 |
| 6314 | CTAGCCCCCACCGCCTTAGA | 663 |
| 6315 | TAGCCCCCACCGCCTTAGAG | 664 |
| 6316 | AGCCCCCACCGCCTTAGAGT | 665 |
| 6317 | GCCCCCACCGCCTTAGAGTG | 666 |
| 6318 | CCCCCACCGCCTTAGAGTGT | 667 |
| 6319 | CCCCACCGCCTTAGAGTGTC | 668 |
| 6320 | CCCACCGCCTTAGAGTGTCA | 669 |
| 6321 | CCACCGCCTTAGAGTGTCAG | 670 |
| 6322 | CACCGCCTTAGAGTGTCAGT | 671 |
| 6323 | ACCGCCTTAGAGTGTCAGTT | 672 |
| 6324 | CCGCCTTAGAGTGTCAGTTA | 673 |
| 6325 | CGCCTTAGAGTGTCAGTTAC | 674 |
| 6326 | GCGTGGACACACGCCCCTAG | 647 |
| 6327 | AGCGTGGACACACGCCCCTA | 646 |
| 6328 | AAGCGTGGACACACGCCCCT | 645 |
| 6329 | CAAGCGTGGACACACGCCCC | 644 |
| 6330 | GCAAGCGTGGACACACGCCC | 643 |
| 6331 | GGCAAGCGTGGACACACGCC | 642 |
| 6332 | TGGCAAGCGTGGACACACGC | 641 |
| 6333 | TTGGCAAGCGTGGACACACG | 640 |
| 6334 | TTTGGCAAGCGTGGACACAC | 639 |
| 6335 | TTTTGGCAAGCGTGGACACA | 638 |
| 6336 | TTTTTGGCAAGCGTGGACAC | 637 |
| 6337 | CTTTTTGGCAAGCGTGGACA | 636 |
| 6338 | TCTTTTTGGCAAGCGTGGAC | 635 |
| 6339 | ATCTTTTTGGCAAGCGTGGA | 634 |
| 6340 | AATCTTTTTGGCAAGCGTGG | 633 |
| 6341 | TAGACACTTCGGTGAATCGTGCCGC | 1598 |
| 6342 | AGACACTTCGGTGAATCGTG | 1599 |
| 6343 | GACACTTCGGTGAATCGTGC | 1600 |
| 6344 | ACACTTCGGTGAATCGTGCC | 1601 |
| 6345 | CACTTCGGTGAATCGTGCCG | 1602 |
| 6346 | ACTTCGGTGAATCGTGCCGC | 1603 |
| 6347 | CTTCGGTGAATCGTGCCGCT | 1604 |
| 6348 | TTCGGTGAATCGTGCCGCTA | 1605 |
| 6349 | TCGGTGAATCGTGCCGCTAT | 1606 |
| 6350 | CGGTGAATCGTGCCGCTATG | 1607 |
| 6351 | GGTGAATCGTGCCGCTATGA | 1608 |
| 6352 | GTGAATCGTGCCGCTATGAA | 1609 |
| 6353 | TGAATCGTGCCGCTATGAAC | 1610 |
| 6354 | GAATCGTGCCGCTATGAACA | 1611 |
| 6355 | AATCGTGCCGCTATGAACAC | 1612 |
| 6356 | ATCGTGCCGCTATGAACACA | 1613 |
| 6357 | TCGTGCCGCTATGAACACAG | 1614 |
| 6358 | CGTGCCGCTATGAACACAGA | 1615 |
| 6359 | GTGCCGCTATGAACACAGAT | 1616 |
| 6360 | TGCCGCTATGAACACAGATG | 1617 |
| 6361 | GCCGCTATGAACACAGATGT | 1618 |
| 6362 | CCGCTATGAACACAGATGTA | 1619 |
| 6363 | CGCTATGAACACAGATGTAC | 1620 |
| 6364 | CTAGACACTTCGGTGAATCG | 1597 |
| 6365 | ACTAGACACTTCGGTGAATC | 1596 |
| 6366 | CACTAGACACTTCGGTGAAT | 1595 |
| 6367 | CCACTAGACACTTCGGTGAA | 1594 |
| 6368 | GCCACTAGACACTTCGGTGA | 1593 |
| 6369 | TGCCACTAGACACTTCGGTG | 1592 |
| 6370 | CTGCCACTAGACACTTCGGT | 1591 |
| 6371 | TCTGCCACTAGACACTTCGG | 1590 |
| 6372 | ATCTGCCACTAGACACTTCG | 1589 |

```
Hot Zones (Relative upstream location to gene start site)
  1-750
  900-1700
2550-2900
4150-4500
```

### Examples

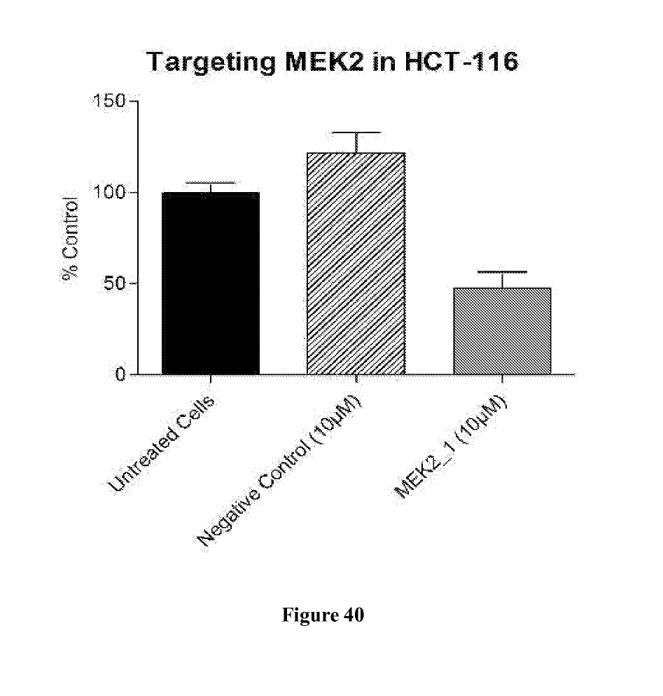

In FIG. 40, In HCT-116 (human colorectal carcinoma cell line), MEK2_{—}1 (224) produced statistically significant (P<0.05) inhibition at 10 μM compared to the untreated and negative control values. The MEK2 sequences MEK2_{—}1 (224) fits the independent and dependent DNAi motif claims.

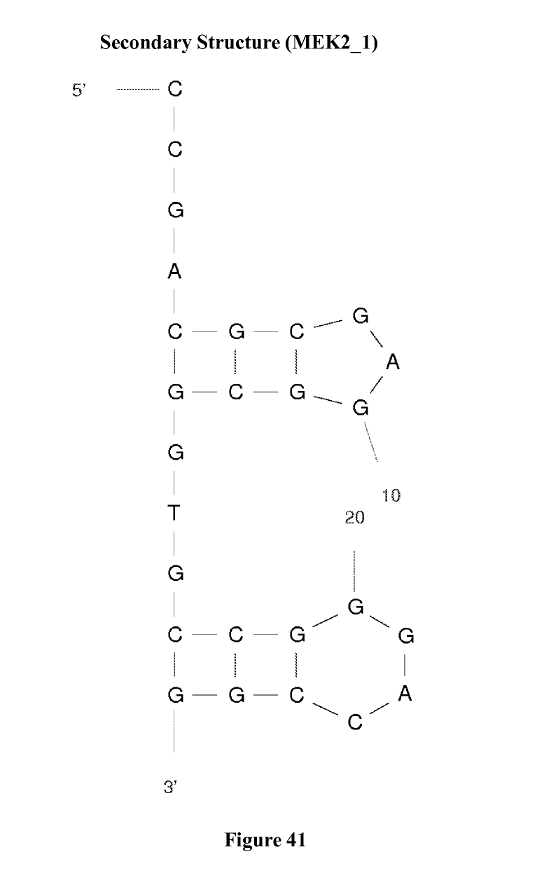

The secondary structure for MEK2_{—}1 (224) is shown in FIG. 41.

Genetic Code (5′ Upstream Region)

(SEQ ID NO: 11970)

GGAACTACAGGTGCCCGCCACCACGCCTGGCTAATTTTTTTGTATTTTTA
GTAGAGACAGGGTTTCACTGTGTTAGCCAGGATGGTCTCTGGTCTCGATC
TCCTGACCTCGTGATCTGCCTGCCTCAGCCTCCCAAAGTGCTGGGATTAC
AGGCGTGAGCCACCGCGCCCGGCCTTGTATTTTTAGTAGAGACAGGGTTT
GTCCATGTTGGTCAGGCTGGTATCGAACTCCCGACCTCAGGTGATCCACC
CGCCTCGGCCTCCCAAAGTGCAGGGATTATAGGCATGAGCCACCACATCT
GGTCTTCTTCTTTTTTTTTTTTTTTTTGAGACAGAGTCTCCCTCAGGCTG
GAGTGCGGTGGCACGATCTTGGCTCACTGCAACCTCCACCTCTCAGGTTC
AAGTAATTCTCGTGCCTCAGCCTCCCAAGTAGCTGAGACTACAGGCACCT
GCCACCATGCCCAGCTAATTTTTTTTTTTTTTCCGAGATGGAGCCTTACT
CTGTTGCCCAGGCTGGAGTGCAGGGGCACAATCTTGGCTCACTGCAACCT
CCACCTCCGGGGTTCAAGCAGTTCTCCTGCCTCAGCCTCCCGAGTAGCTG
GGATTACAGGTGCCCACCACCATGCCCGGCTAATTTTTGTGTGTTTTTAG
TAGAGACGGGGTTTCACCATGTTGGTCAGGCTGGTCTTGAACTCTTGACC
TCAGGTGATCTGCCCACCTCGGCTTGCCAAAGTGCTGTGATTACACCCGT
GACCAGCCTAATTTTTGTATATTTAGTAGAGATGGGGTTTCACCATGTTG
GCCAGGCTGGACTCGAACTCCTGACCTCAAGTGATCACCTGCTTTGGCCT
CCCAAAGTGCTGGGATTGCAGGTGTGAGCCACCACACCCGGCCTCTCCTT
ATTTTAATGGCTCATTGTTAAACATTTACCAGCTCACTACTGCTGGGTGC
AGAGGAAGAGAATGAACTAAAAAGGCAGTGAACAGACTTTCTGGAGTAAG
GGGAAGTGTTACATGGATGTATAGAGTTGTAATAATCCAAGAAATTGAAC
TTCAGAAACTTGTGCATTAATAGGTGAGTGCAGTGGCTCACGGCTCTAGT
CCCAGCACTGCTGAGGACGAGGCAGGAGGATCGCTTGAGCCTAGGAGTGT
GAGACCAGCCTGAGTGACATGGAGAAACCCTGTCTGGACAAAAAATACAA
AAATTAGCCGAGTGTGGTGGCGTATGTTTGTAGCCAGGGCTACTAGGGAG
GCTGAGGTGGGAGAATCGCTTGAGCCAGGGAGGTGGAGGCTGCAGAGAGT
TATGATCGTGCCACTGCACTCCAGCCTGAGGCCTGGGTGACAGAGTCAGA
ACTTGTCTTAAAAAGAAAAAAAAAGCCTAAAATAGGATAAAATGGGAGAA
AGATTGCTAGGCAAAACAGAAGGAACATGGAAATAGCCCTGTCTCTGAAA
GGGCCTGTCCTTATTTGAGGCCACATATGCATCCATCTGAATTTTGGACA
AGCGGGTGGGAGCGATGAGAAGTAAAACTGAAAGGCCCAGATTGTAAAAA
CCCAGGAGCAGGCTTCCCCAGGAGCAGTGTTTTGTTTGTTGTTTTGTTTT
GTTTTGTTTTTTTCGAGATGGAGTCTCGGTCGGTCGCCCAGGCTGGAGTG
CAATGGCGTGATCTCGGCTCACTGCAACCTCCACCTCCCGGGTTTAAGCG
ATTCTCCTGCCTCAGCCTCCCGAGTAGCTGGGACTACAGGCACGCATCAC
CACACCCAGTTAATTTTTGTATTTTTAGTAGAGACGGGGTTTCACCATGT
TGGCCAGGATAGTCTCAATCTCTTGACCTCATGATCCACCTGCCTTGGCT
TCCCAAAGTGCTGGGATTACAGGCGTGAGCCACCGCGCCCGGCCAGTTGG
TTGGTTTTGTTTTTTGAGCGTGAGTCTGGCTCTGTCGCCCAGGCTGGAAT
GCGATGGCACAATCTCGGCTCACTGCAACCTCCGCCTCCGGGGTTCAGTT
ATCCCACCTCAGCCTCCCTAGTAGCTGGAATTACAGCCACCCGCCACCAC
ACCTGTGTAATTTTTGTATTTTTAGTAGAGACGAGGTTTCACCATGTTGG
CCAGGTTGGTCTCGAACTCCTGACCTCAAGTGATCAGCCCACCTCAGCTT
CCCAGGGTCCTGGGATTACAGGTGTGAGCCACGGCACCTGGCAAAAAATT
AAATTTTTTTTTGTTCTGTTTTATTGGAGACGGAGTCTTACTTTGTCGCC
CAGGCTGGAGGGCAGTGGTGCAATCTTGGCTCACTGCAACGTCTGCCCCC
CGGGTTCAAGCGATTCTCCTGCCTCAGCCGCCTGAGTAGCTGAGACTATA
GGCACACACCGCCAGGCCTGGCTAATTTTTGTATTTTATTTATTTATTTG
TTTGTTTGTTTGTTTGTTTGATTTTTTTGAGACGAAGTCTCGCTCTTGTC
TCCCAAGCTGGAGTGCAATGGCGTGATCTTGGCTCACTGCAACCTCTGCC
TCCCGGGTTCAAGCAATTCTTCTGCCTCAACCTCGCGAGTAGCTGGGATT
ACAGGCACGCGCCACCATGCCCGGCTAATTTTTGTATTTTTTTGTTTTAG
TAGAGACGGGGTTTCACCATGTTGGCCAGACTCGTCTTCAACTCCTGACC
TCAGGTGATCCACCCGCCTCGACCTCCCAAAGTGCTGGGATTACAGGCGT
GAGCCACCGCGCCCAGCCTATGACCTTTCTTATAAAGTGGTACGGCTATT
GTATTAAATAGTAAGGTGGTGCTTCAAAAAGTTCAACATAGAATTACCAT
ATAATCCAGTAATTCCTCTTCAGAACATATACCCAAAAGAACTCAAGGCA
AGGACTCAAACAGATATTTGTACATCTGTGTTCATAGCGGCACGATTCAC
CGAAGTGTCTAGTGGCAGATAAATGGATAAGCAAAATATAGTCCATGCAC
ACAATAGAATATTATTCAGCCTTAAAAAGGAGGAAAATCCTGACTGGGTG
CGGTGGCTCACGCCTGTAATCCCAGCACTTTGGGAGATCGAGGCGGGTGG
ATCACGAGGTCAGGAGATGGAGACCATCCTGGCTAACACGGTGAAACCCC
GTCTCTACTAAAAATACAAAAAAATTAGCCGGGCATGGTGGTGGGCACCT
GTAGTCCCAGCTACTCGGGAGGCTGAGGCAGGAGAATGGCGTGAACCCGG
GAGGCAGAGTTTGCAGTGGGCCGAGATCGCACCACTGCACTCCAGCCTGG
GTGACAGAGCGAGACTCCGTCTCAAAAAAAAAAAAAGGGAAATTTTTCTT
TTTTTTTTTTTTTCTGCTCTTTTTTGGAGCAGGGCTACCCGATTGGAAGT
ATGCCCGGAGTAGTCAAGTGGGTAAATTCTAACACAGGTTACAACGTGGA
TCTAACACAGCTACAACAGGCACCTTGAGGACGTGGCCCTCAGTGAAATA
TGCCAGCCACAAAGGGACAAAACCTGTGTGATCCTACTCATATGAAGTCC
CTAGAATCATCAGATTCACAGGAAGTACGACGTTGGGTTCCAGGGGCTGG
GGAGGGGGATAGGGAGTGAGGTTTCATAGGGGACAGTGTTTCAGTTTCGG
AAGATGAGAAAATTCTGGAGATGGTGGTGGTGGTGGTTGCTTAATGCCGC
TGAGCTGTGCATTTAGAAATGGTTAAAATGACAAGTTTTATGTTATGTGT
ATTTTATAATAAAAATGTTTCAACATGCGCATAGTAATATATGCAATTTT
ATTTGTCAATTAAAATAAATTTTAAAAATGTTTTAGAGTGGCCTTGTTCT
GATGAAGGAGGGGGAGTAACTGACACTCTAAGGCGGTGGGGGCTAGGGGC
GTGTGTCCACGCTTGCCAAAAAGATTAAATGGACTCTGGGTGGGTCTCGT
CCACTGTTCTGGGGTCTTACGGGTTCTCTCAGCCCCAGCCTGGGGCACCA
CAGGCTCTCAGGAGTCTGGCTACCCTGCCCACCTGTGCACGACCATCACC
CCAGCCTTCATCCCTCCGTCTCCTCCCCTGCTCCCGCGCCTCATGCCCCA
AGCGCTTGGGCACACGCGCCGTGGTAAGGCAAGCGAGGGCGCCCGGTCCC
GGCACCGCCTCGCGTCGGTCTCCGCCCCTTTCCCCTCCGAAAGGCGGCCT
TGTGCTGCTGCGCAGGCGCGGCGGCTGGGGGTGGGGTCCATCGCGGCTCC
CGATCCCGTTATCGCGAGAAGCCGGTCCGCGATCTTGTGGCCGCCCCTCC
CCTCCCCCTGCCTCTCGGACTCGGGCTGCGGCGTCAGCCTTCTTCGGGCC
TCGGCAGCGGTAGCGGCTCGCTCGCCTCAGCCCCAGCGCCCCTCGGCTAC
CCTCGGCCCAGGCCCGCAGCGCCGCCCGCCCTCGGCCGCCCCGACGCCGG
CCTGGGCCGCGGCCGCAGCCCCGGGCTCGCGTAGGCGCCGACCGCTCCCG
GCCCGCCCCCTATGGGCCCCGGCTAGAGGCGCCGCCGCCGCCGGCCCGCG
GAGCCCCGATG

22. CD4. CD4 (cluster of differentiation 4) is a glycoprotein found on the surface of immune cells such as T helper cells, monocytes, macrophages, and dendritic cells. In humans, the CD4 protein is encoded by the CD4 gene (Isobe et al., Proc. Natl. Acad. Sci. U.S.A. 1986; 83 (12): 4399-402). CD4+ T helper cells are white blood cells that are an essential part of the human immune system, often referred to as CD4 cells, T-helper cells or T4 cells. These helper cells send signals to other types of immune cells, including CD8 killer cells which in turn destroy and kill the infection or virus. If CD4 cells become depleted, for example in untreated HIV infection, or following immune suppression prior to a transplant, the body is left vulnerable to a wide range of infections that it would otherwise have been able to fight.

HIV-1 uses CD4 to gain entry into host T-cells and achieves this by binding to the viral envelope protein known as gp120 (Kwong et al., Nature 393 (6686): 648-59). The binding to CD4 creates a shift in the conformation of gp120 allowing HIV-1 to bind to a co-receptor expressed on the host cell. These co-receptors are chemokine receptors CCR5 or CXCR4. Following a structural change in another viral protein (gp41), HIV inserts a fusion peptide into the host cell that allows the outer membrane of the virus to fuse with the cell membrane. CD4 is also expressed in neoplasms derived from from T helper cells, e.g. peripheral T cell lymphoma and related malignant conditions and has been associated with a number of autoimmune diseases such as vitiligo and type I diabetes mellitus (Zamani et al., Clin. Exp. Dermatol. 35 (5): 521-4).

Protein: CD4 Gene: CD4 (Homo sapiens, chromosome 12, 6898638-6929976 [NCBI Reference Sequence: NC_{—}000012.11]; start site location: 6909305; strand: positive)

Gene Identification

| | |
|---|---|
| GeneID | 920 |
| HGNC | 1678 |
| HPRD | 01740 |
| MIM | 186940 |

Targeted Sequences

| | | Relative |
|---|---|---|
| | | upstream |
| | | location |
| Sequence | | to gene |
| ID No: | Sequence (5′-3′) | start site |
| 6373 | GAGCCACTGCGCCCGGCCTCATTAAGGGCAT | 12485 |
| 6406 | CGAACAACTTCATTACAATTCGACAAGCGC | 13299 |
| 6407 | CGTAGTTAAGCGTGTACCAGCCCAAGGC | 13189 |
| 6421 | GAGCGGTGACCGTGTCTGTCTTAG | 13751 |
| 6447 | CGGTTTGCAGATTCCAGACCCGATGGACG | 15100 |

Target Shift Sequences

| | | Relative |
|---|---|---|
| | | upstream |
| | | location |
| Sequence | | to gene |
| ID No: | Sequence (5′-3′) | start site |
| 6373 | GAGCCACTGCGCCCGGCCTCATTAAGGGCAT | 12485 |
| 6374 | AGCCACTGCGCCCGGCCTCA | 12486 |
| 6375 | GCCACTGCGCCCGGCCTCAT | 12487 |
| 6376 | CCACTGCGCCCGGCCTCATT | 12488 |
| 6377 | CACTGCGCCCGGCCTCATTA | 12489 |
| 6378 | ACTGCGCCCGGCCTCATTAA | 12490 |
| 6379 | CTGCGCCCGGCCTCATTAAG | 12491 |
| 6380 | TGCGCCCGGCCTCATTAAGG | 12492 |
| 6381 | GCGCCCGGCCTCATTAAGGG | 12493 |
| 6382 | CGCCCGGCCTCATTAAGGGC | 12494 |
| 6383 | GCCCGGCCTCATTAAGGGCA | 12495 |
| 6384 | CCCGGCCTCATTAAGGGCAT | 12496 |
| 6385 | CCGGCCTCATTAAGGGCATT | 12497 |
| 6386 | CGGCCTCATTAAGGGCATTC | 12498 |
| 6387 | CGAGCCACTGCGCCCGGCCT | 12484 |
| 6388 | ACGAGCCACTGCGCCCGGCC | 12483 |
| 6389 | CACGAGCCACTGCGCCCGGC | 12482 |
| 6390 | GCACGAGCCACTGCGCCCGG | 12481 |
| 6391 | GGCACGAGCCACTGCGCCCG | 12480 |
| 6392 | AGGCACGAGCCACTGCGCCC | 12479 |
| 6393 | CAGGCACGAGCCACTGCGCC | 12478 |
| 6394 | ACAGGCACGAGCCACTGCGC | 12477 |
| 6395 | TACAGGCACGAGCCACTGCG | 12476 |
| 6396 | TTACAGGCACGAGCCACTGC | 12475 |
| 6397 | ATTACAGGCACGAGCCACTG | 12474 |
| 6398 | GATTACAGGCACGAGCCACT | 12473 |
| 6399 | GGATTACAGGCACGAGCCAC | 12472 |
| 6400 | GGGATTACAGGCACGAGCCA | 12471 |
| 6401 | TGGGATTACAGGCACGAGCC | 12470 |
| 6402 | CTGGGATTACAGGCACGAGC | 12469 |
| 6403 | GCTGGGATTACAGGCACGAG | 12468 |
| 6404 | TGCTGGGATTACAGGCACGA | 12467 |
| 6405 | GTGCTGGGATTACAGGCACG | 12466 |
| 6406 | CGAACAACTTCATTACAATTCGACAAGCGC | 13299 |
| 6407 | CGTAGTTAAGCGTGTACCAGCCCAAGGC | 13189 |
| 6408 | GTAGTTAAGCGTGTACCAGC | 13190 |
| 6409 | TAGTTAAGCGTGTACCAGCC | 13191 |
| 6410 | AGTTAAGCGTGTACCAGCCC | 13192 |
| 6411 | GTTAAGCGTGTACCAGCCCA | 13193 |
| 6412 | TTAAGCGTGTACCAGCCCAA | 13194 |
| 6413 | TAAGCGTGTACCAGCCCAAG | 13195 |
| 6414 | AAGCGTGTACCAGCCCAAGG | 13196 |
| 6415 | AGCGTGTACCAGCCCAAGGC | 13197 |
| 6416 | GCGTGTACCAGCCCAAGGCA | 13198 |
| 6417 | CGTGTACCAGCCCAAGGCAC | 13199 |
| 6418 | ACGTAGTTAAGCGTGTACCA | 13188 |
| 6419 | TACGTAGTTAAGCGTGTACC | 13187 |
| 6420 | GTACGTAGTTAAGCGTGTAC | 13186 |
| 6421 | GAGCGGTGACCGTGTCTGTCTTAG | 13751 |
| 6422 | AGCGGTGACCGTGTCTGTCT | 13752 |
| 6423 | GCGGTGACCGTGTCTGTCTT | 13753 |
| 6424 | CGGTGACCGTGTCTGTCTTA | 13754 |
| 6425 | GGTGACCGTGTCTGTCTTAG | 13755 |
| 6426 | GTGACCGTGTCTGTCTTAGT | 13756 |
| 6427 | TGACCGTGTCTGTCTTAGTT | 13757 |
| 6428 | GACCGTGTCTGTCTTAGTTA | 13758 |
| 6429 | ACCGTGTCTGTCTTAGTTAG | 13759 |
| 6430 | CCGTGTCTGTCTTAGTTAGC | 13760 |
| 6431 | CGTGTCTGTCTTAGTTAGCA | 13761 |
| 6432 | AGAGCGGTGACCGTGTCTGT | 13750 |
| 6433 | CAGAGCGGTGACCGTGTCTG | 13749 |
| 6434 | CCAGAGCGGTGACCGTGTCT | 13748 |
| 6435 | GCCAGAGCGGTGACCGTGTC | 13747 |
| 6436 | GGCCAGAGCGGTGACCGTGT | 13746 |
| 6437 | AGGCCAGAGCGGTGACCGTG | 13745 |
| 6438 | CAGGCCAGAGCGGTGACCGT | 13744 |
| 6439 | ACAGGCCAGAGCGGTGACCG | 13743 |
| 6440 | CACAGGCCAGAGCGGTGACC | 13742 |
| 6441 | TCACAGGCCAGAGCGGTGAC | 13741 |
| 6442 | CTCACAGGCCAGAGCGGTGA | 13740 |
| 6443 | GCTCACAGGCCAGAGCGGTG | 13739 |
| 6444 | AGCTCACAGGCCAGAGCGGT | 13738 |
| 6445 | TAGCTCACAGGCCAGAGCGG | 13737 |
| 6446 | CTAGCTCACAGGCCAGAGCG | 13736 |
| 6447 | CGGTTTGCAGATTCCAGACCCGATGGACG | 15100 |
| 6448 | CCGGTTTGCAGATTCCAGAC | 15099 |
| 6449 | ACCGGTTTGCAGATTCCAGA | 15098 |
| 6450 | CACCGGTTTGCAGATTCCAG | 15097 |
| 6451 | CCACCGGTTTGCAGATTCCA | 15096 |
| 6452 | CCCACCGGTTTGCAGATTCC | 15095 |
| 6453 | GCCCACCGGTTTGCAGATTC | 15094 |
| 6454 | GGCCCACCGGTTTGCAGATT | 15093 |
| 6455 | GGGCCCACCGGTTTGCAGAT | 15092 |
| 6456 | TGGGCCCACCGGTTTGCAGA | 15091 |
| 6457 | TTGGGCCCACCGGTTTGCAG | 15090 |
| 6458 | TTTGGGCCCACCGGTTTGCA | 15089 |
| 6459 | CTTTGGGCCCACCGGTTTGC | 15088 |
| 6460 | GCTTTGGGCCCACCGGTTTG | 15087 |
| 6461 | AGCTTTGGGCCCACCGGTTT | 15086 |
| 6462 | TAGCTTTGGGCCCACCGGTT | 15085 |
| 6463 | CTAGCTTTGGGCCCACCGGT | 15084 |
| 6464 | TCTAGCTTTGGGCCCACCGG | 15083 |
| 6465 | CTCTAGCTTTGGGCCCACCG | 15082 |

Hot Zones (Relative upstream location to gene start site)
12350-12500
13100-13300
13700-13800
15000-15200

### Examples

Genetic Code (5′ Upstream Region)

(SEQ ID NO: 11971)

ATCTAATCTATCTATATCTGTCTATCTATCTTTATGTATCTATCTTATCT
ATTGATCTATCTATCTTTTTTTTTTTTTGAGACAGAGTCACTCTGTCACC
CAGGCTGGAGTGCAGTGGCACGATCTCGGCTCACTGCAACCTCCGCCTCC
CGGGTTCAAGCGATTCTCCTACCTCAGCCTCCTCAGTAGCTGGGACTACC
CACCACCACTCCTGGCTAATTTTTGTATTTTCAGTAGAGATAGGGTTTCA
CTATGTTGGCCAGGCTGGTCTCCAACTCCTGACCTAAAGTGATCCACCCA
CCTTGGTTTCCCAAAGTGCTGGGATTACAGGCGTGAGCCACCGTGCCTGG
ACATATATCTATCTTTTTTTTTTTTGAGATGGAGTCTCGCTCTGTTGCCC
AGGCTGGAGTGCAGTGGCGTGATTTCGGCTCACTGCAACCTCCGCCTCCC
GGGTTCAAGTGATTCTCCTGCCTCAGCCTCCCAAGTAGCTGAGATTACAG
ACGTGCGTCACCATGCCCAGCTAATTTTTGTATTTTTAGTAGAGATGGGA
TTTCACTATGTTGGCCAGGCTGGTCTCGTACTCCCGACCTCAGGTGATCC
ACTTGCCTTGGCCTCCCAAAGTGCTGGAATTACAGGTGTGAGCCACTGCA
TCCGGCCTTATATATCTATCTTGTCTGTCTGACTGTCTAATCTAATTCAT
CTATTTTATCTGTTTATCTTATCTATCATCTATTTATCTAATCTATCTGT
CTGTATGTCTGTTTTTTTTTTGTTTTTTTTTTTTTTTTGAGATAGAGTCT
TGCTCTGTCGCCGAGGCTGGAGTGCGGTGGCGCGATCTCAGCTCACTGCT
GAACCTCCGCCTCCTGGGTTCTAAGCGATTCTCCTGCCTCAATCTTTGGA
GTAGCTGGGATTACAGGCCCGTACCACTGTGCCCGGCTAATTTTGTATTT
TTAGTAGAGAAGGGTTTCACCATGTTGGTCAGGCTTGTATTGAACTCCTG
ACCTCAGGTGATCTACCCGCCTAAGCCTCCCAAAGTGCTGGGAGTACAGG
TGTGAGCCACTGTGTCTGTCCCTAAATGTCTGTCTCTATCTATCTATCTA
TCTATCTATCTATCTATCTATCTAATCTATCTTTCTGTCTAACCTAATCT
ATTTTATCTATCTTATTCATCATCTATCTAATCTGTCTGTATGTTTATCT
AATCTATTTACCTAATCTATCAATCTATCATCTAATCTATCTAATCTGTC
TATCTAATCTATTTTATCTATCTATCTATCTGTCCATCCATCTATCTACC
TACCTGTCTATCTCAAGCACCTACCACGTATTAAGCCCTGGCTACCTCCT
CTTCCAGGCAGATGGAGTAACTGGAGGCAGCTAACAAAGATGGAGTCACT
TTTCTTATCTTCTCCTAAACCACCGTAAGAGGACCAAGCCCCCACACCTT
CTGAGTGCCCCATTCCTCTCCACAGATTGTGTCTTAGTGCCCAGCAGGAA
ACACAGTCCACCTCCCATGGTTCAAGAGATTGTAGAAAGGGGGTTATTCA
CATAGGTTAAGGGAATCAATCAATTTGAAGCACAGACACTATTAACAGCA
GGAAGAGTCCTGAAGAAGTGAAAATGGTGTTTCTGGAACCCAGAGAGTGC
TTGCACTCTGGATAAGGGGCCACCCCACAGAAGCTGTGGAGGGGCAGGGC
TGCAGGTGAGGATGAACACACAGCTATTGACAGAAAATATGCCCAGGGCA
GGGATAGAGTAGGAAAAATATCCCAGCTTCTTTCCCCCACCCTTCCATCT
CATCTCTGAAAGGCACTTCCCACTGGCCAGCCCCGACTGGTGCTGGAGGG
CAAGAGAGCCTATGAGCCATGTGTGGCTGTCAGCCCCTTGGTGGAGAGCC
ACAGACAGGATGGAGAGTGGCTGGCAGGGCCCCGTGGGGATGAACAGCTT
GGATTGGGGCGACTGGGCTTCATCCAGGCTGGGCTGGATGTGTGCATACA
TTTCAGTGACCCGTTTTAGAAACAGAATTAATATGGTGAATAGAGAAAGA
AGAAATCAGTGACTTTCGCTCCTCCATACAATTCAATTTGGCTTAAGTTA
GCCAAAGCCATACCAAGTCCTCTCTCTATGTCTCAGCTGCTGCCAGGCTT
GTGGTGGCCACACAGCTGGCTAGACTGTCATCTCTGTCCTCAAGGGGCTC
AAGCTAGAGGAGGAGAGTTGAGAAACCAAATCACTATACACAAAGTAGAA
GGTGGAACACACCCAGGAGCATGTCAACGGGGTGCTGTGGGACTTCAGAG
TAGGCAGATCGTCACCAAGCTTCAACGGCAAAGATGCCACTGGGGGAAAG
AAGGACCAAGCTTGGAAGACAGAGTAAGTCTGGAGGCAAGATCTTGTCTC
ACCAGCAGGGGCCAGGTCCATGGTGACACCTTCCCCAGGCAGTCACCTCT
CTGAGCCCACTTTATATCCTAGGCCTGGATTCAAAGACACTTGAGCCCTG
CTCCAGCCTTCCTTTGAGGTGCTATCTTGGTGCCTTTCCTATAATCACTG
CTCCAGTCCCATGTCATCTGGTCCCCAGTTACCACATCAAGCTTCCCGAA
GCTCCACACAGACCATGCCACATCTTTACCAAAAAATCAGCAGTGGGTCC
CCTCACCTCCAGGACAAAGCTCCAGCTCTTCGACCTGCCTGTCAATATTT
GCAATCACTGCCTGCACAAATTAGCTGGGTGTTGTCATGAAAGGATCACT
TGAGCCCAGGAGTTCCAGGCTGCAATGACCTATGATTGAACCACTGCACT
CTGGCCTGGGTGACAGAGTGGATCTAAACTAAAAATAAAAAGATTTACAG
TCAAGCCTCAAAGGCTTTTCCCATACCTTCTTCCACCATCACCTCCCTGA
GCCCTCTCTTTCCTCCGAAGCCTCCTCGCACATCCCTACCACCTTTGCAC
ACCTCAGAATGGGGACACCTCTCCCCTTTCCTCTCCATCTAACTTATGGT
TTTCAAACTTGAGCGTGATCAGTTACCTGGAGATTTGTGAAAACCCAGAT
GACTAGACCCACCCCCAGTTTCTGATTCAGCAGGTCTGGGGTGGGGCCGA
GGATCTGCATTTCTAACAAGTTCCCAGGTCATGTTGCCGCTGCTACTGAT
CCAGGACTTTGGGAATCGCTCCTCTAATCTACAGCTGTCCATTCCCCATG
GTCCATTCAGAGCCTCTCTGCCCTGCCCCCACCACCCCCAGTCTCGCCTG
TCTGCCAAGCGCACAGGAAACTCTCCTTCATCCAAACCCTGGACCAACGC
CTTCTGCTTGGCCCACTCAGAGGCCTTGTAGGGTTGGTCTGATATTGGAC
AGAGAAATGGCCCTCTGCTCTTTCTCCCCTGACCTCTCTGAAGGGGGCCT
GCCCCTCCACACCTGTGGGTATTTCTCGCAAGGTGGAGACAAGAGACTGA
GAAAAGAAATAAGACACAGAGAAAGTATAGAGGAATAAAAGTGGGCCCAG
GGGACCGGCGCTCAGCAAGTGAGGACCTGCACCGGTGCTGGTCTCTGAGT
TCCCTCAGTATTTATTGATCACTATCTTTACTATCTCCGCGAGGGGAATG
TGGTGGGGCTATAGGGTGAAGGTGAGGAGAGGGTCAGCAGAAAAACATAT
GAGCAAAGACTCTGTGTCATAAATAAGTTTAAGGAAAGGTGCTGTGCCTG
GATGTGCTAGATTTATGTTTAACTTTACACAAACATCTCAGTGTAGTAAA
GAGTAACAGAGCAGTATTGCCGCCATGATGTCTCGCCTCCAGACATAAGG
CAGTTTTCTCCTCTCTCAAAATAGAATGTATGATCGGTTTTACACCGGGT
CATTCCATTCCCAGGGACGAGCAGGAGACAGATGCCTTCCTCTTATCTCA
ACCGAATAGAGGCCTTCCTCCTTCACTAATCCTCCTCAGCACAGACCCTT
TACGGGTGTCGGGCTGGGGGGCTGTAAGGTCTTTCCCTTCCCATGAGGCC
ATATCTCAGGCTGTCTCAGTGGGGGGAAACCTGGACAATACCTAGGCTTT
CTCGGGCAGGGGTTCCTGCGGCCTTCCACAGTGTATTGTGTCTCTGGTTA
ATAGAGAACGGAGAATGGTGATGACTTTCACCAAGCACACTGCCTGCAAG
AACTTTTCTTTTTTTTTTTTTTTGAGACAGAGTCTTGCTCTGTCGCCCAG
GCTGGAGTGCAGTGGCGCGATCTCGGCTCACTGCCACCTCTGCCTCCCGG
GTTCACGCCATTCTCCTGCCTCAGCCTCCCGAGTAGCTGGGACTACAGGC
GCCCGCCACCACGCCCGGCTAATTTTTTTGTATTTTTTTAGTAGAGATGG
GGTTTCACCGTGTTCACCAGGATGGTCTCGATCTCCTGACCTCATGATCC
GCCCGCCTTGGCCTCCCAAAATGCTGGGATTACACGTGTGAGGCAAGAAC
TTTTTAAAAGTGCATCTTGCGCAGCCCTAGATCCATTAAACCTTGATTCA
ATACAGGACATGTTTTTGTGAGCACAGGGTTGGGACAAAAGTTACAGATT
AACAGCATCTCAAAGCAGAACAATTTTTCTTAGTACAGATCAAAATGGAG
TTTCTTATGTCTTCCTTTTTCTACATAGACACAGTAACAATCTGATCTCT
CTTTCTTTTCCCCATACCTCTCACGCTGTATCAGGCCCCAATTCTTGGGA
ACGTCACCTTAGAACTGTCCCACACATTTCTACAGCCACTTGGCTCAGGC
CCTTTGCTGACCAGGATGGTTGCAGTTCTGCCTTTGGTGCCTCGCCTCCT
CCAGTTCTTTCACTCAGCAGCTGCAGGGGTCCACGTGGCAAATCTAATAA
TCTTCTTCTCTATAGAAAATCCTCTGCTGGCTCTCTAGTGCCCAGGATCC
AGTCCCAGCATCTCAGCACGGCCTTCAAGCATTTCCACGTCCTGGCCTGG
CTCCATGGTCTCCCCGCCAATTTGCCACCTTCTCCATGCATCCTTTTCTG
ATCCCCTCCTCACTCATCCCAGCAAAGAACCCCCTCCTGGCCTGAGCATA
GCATTTCGTGGTGTGTATCTCAGAGCATCCAGTTAGGGGTGTGCAAGTTT
ACTTTGTTACTGGCTGATGTTGTGAAGTCCCAAGTTGTTGGTGCCGCAAA
CAAAAAATTGGACATGACACACACAAATAGCAAAGCAGCAAAAGTTTATT
AAGCACAGTACGATCCACTATGGATCAAGGATGACCTGCGAATGGTATCA
GCATCACTTTGCTATATTTCATGGCCTTTTCTATGTGTTTTTTTTCTCTT
TTTCCTCAAGCTGCCTAAGCTTTAGCCAGCATGTGCCTTTTGGTTGACAG
GTGGGTTGCTTAGTTTCTTGGCCTCTGTGTGTTTACGTGTCATTTCCTTC
CCATAGTTTTAAGTACATGCATGATATGCACTCTGTAGGCATGAACCTTA
AGTAGCTAATTACTATACGGGGTCATTTTGAGGATATCTTTTCTCTGTAG
TACATGTGCATCTTTTTTTGCAGTGGTGCAATCTTGGCTCACTGCAACCT
CCTCCTCCCTGGTTCAAGTGATTCTCCTGCCTCAGCTTCCTAAGCACCTG
AGACTACAGGTGCATGCCACCACGCCCGGCTAATTTTTGTATTTTTAGTA
GAGATGGGGTTTCACCATGTTGGCCAGGCTGATCTCGAACTCCTGACCGC
AAGTGATCCACCCACCTCGGCCTCCCAAAGCACTGGGATTACAGGCATGA
GCCACCGCACCCAGCCTAGTATATGCCCATCTCTTAGGAGCTGCTCCTAA
CTGGTTTGGTTTGGATCTAGCCAGCCATGGGGCTCCTTATTCACTTATTT
ATCTTCTGTTTTTGCTCACCTGCCTCTTTCTCTTGCTTCTGCTCCTACTC
ATTCCTTCCTTAATCCAACCTCCAATTCCCTCTGCTATTCTCCTGCCTCA
AGTTCACTAGGCTGGCTGCAAGGGTCCTGAGGGAGAGGTTGTGTATCGCC
CCTGTATACTCCAGGTCCAGTAAATGTTTGCTGACTAATGATTGGCATTT
CCCTCAGGCCCTGCCATTTCTGTGGGCTCAGGTCCCTACTGGCTCAGGCC
CCTGCCTCCCTCGGCAAGGCCACAATG

23) WNT1 WNT1 (wingless-type MMTV integration site family, member 1) is a member of the WNT protein family of secreted molecules that are involved in intercellular signaling during development. WNT proteins have been shown to have regulatory roles in the cell fate process and have been associated with tumorigenesis. WNT proteins stimulate either the canonical or non-canonical intracellular signal transduction cascades. WNT proteins bind to the extracellular Frizzled (Fz) receptor family. Binding of WNT to the Fz and low density lipoprotein related protein 5/6 receptor complex, disrupts downstream protein complexes which inhibits the destruction of β-catenin. β-catenin enters the nucleus and complexes with TCF to initiate WNT-related gene expression. WNT1 has been associated multiple cancers including hepatitis B virus-related and hepatitis C virus-related hepatocellular carcinoma, gastric cancer, pancreatic cancer, breast cancer, and lung cancer.

Protein: Wnt-1 Gene: WNT1 (Homo sapiens, chromosome 12, 49372236-49376396 [NCBI Reference Sequence: NC_{—}000012.11]; start site location: 49372434; strand: positive)

Gene Identification

| | |
|---|---|
| GeneID | 7471 |
| HGNC | 12774 |
| HPRD | 01276 |
| MIM | 164820 |

Targeted Sequences

| | | Relative |
|---|---|---|
| | | upstream |
| | | location |
| | | to gene |
| Sequence | | start |
| ID No: | Sequence (5′-3′) | site |
| 6466 | CGCGCGCCCGCCTCACTCAGCTGAGCG | 442 |
| 6537 | CGTCATTCTGTTGCCCTTTGTACCTCG | 1226 |
| 6545 | CGCCACGGGCGCATCCATCCCTCCTGGG | 4454 |
| 6579 | CACCGCCCTCTAGCCGCCTGCGGG | 4960 |
| 6580 | TTGCGGCGACTTTGGTTGTTGCCCGCGACGGT | 34 |

Target Shift Sequences

| | | Relative |
|---|---|---|
| | | upstream |
| | | location |
| | | to gene |
| Sequence | | start |
| ID No: | Sequence (5′-3′) | site |
| 6466 | CGCGCGCCCGCCTCACTCAGCTGAGCG | 442 |
| 6467 | GCGCGCCCGCCTCACTCAGC | 443 |
| 6468 | CGCGCCCGCCTCACTCAGCT | 444 |
| 6469 | GCGCCCGCCTCACTCAGCTG | 445 |
| 6470 | CGCCCGCCTCACTCAGCTGA | 446 |
| 6471 | GCCCGCCTCACTCAGCTGAG | 447 |
| 6472 | CCCGCCTCACTCAGCTGAGC | 448 |
| 6473 | CCGCCTCACTCAGCTGAGCG | 449 |
| 6474 | CGCCTCACTCAGCTGAGCGT | 450 |
| 6475 | GCCTCACTCAGCTGAGCGTC | 451 |
| 6476 | CCTCACTCAGCTGAGCGTCC | 452 |
| 6477 | CTCACTCAGCTGAGCGTCCG | 453 |
| 6478 | TCACTCAGCTGAGCGTCCGG | 454 |
| 6479 | CACTCAGCTGAGCGTCCGGA | 455 |
| 6480 | ACTCAGCTGAGCGTCCGGAG | 456 |
| 6481 | CTCAGCTGAGCGTCCGGAGC | 457 |
| 6482 | TCAGCTGAGCGTCCGGAGCC | 458 |
| 6483 | CAGCTGAGCGTCCGGAGCCC | 459 |
| 6484 | AGCTGAGCGTCCGGAGCCCG | 460 |
| 6485 | GCTGAGCGTCCGGAGCCCGT | 461 |
| 6486 | CTGAGCGTCCGGAGCCCGTC | 462 |
| 6487 | TGAGCGTCCGGAGCCCGTCG | 463 |
| 6488 | GAGCGTCCGGAGCCCGTCGA | 464 |
| 6489 | AGCGTCCGGAGCCCGTCGAG | 465 |
| 6490 | GCGTCCGGAGCCCGTCGAGG | 466 |
| 6491 | CGTCCGGAGCCCGTCGAGGA | 467 |
| 6492 | GTCCGGAGCCCGTCGAGGAC | 468 |
| 6493 | TCCGGAGCCCGTCGAGGACT | 469 |
| 6494 | CCGGAGCCCGTCGAGGACTA | 470 |
| 6495 | CGGAGCCCGTCGAGGACTAG | 471 |
| 6496 | GGAGCCCGTCGAGGACTAGC | 472 |
| 6497 | GAGCCCGTCGAGGACTAGCA | 473 |
| 6498 | AGCCCGTCGAGGACTAGCAT | 474 |
| 6499 | GCCCGTCGAGGACTAGCATC | 475 |
| 6500 | CCCGTCGAGGACTAGCATCC | 476 |
| 6501 | CCGTCGAGGACTAGCATCCG | 477 |
| 6502 | CGTCGAGGACTAGCATCCGC | 478 |
| 6503 | GTCGAGGACTAGCATCCGCC | 479 |
| 6504 | TCGAGGACTAGCATCCGCCA | 480 |
| 6505 | CGAGGACTAGCATCCGCCAG | 481 |
| 6506 | GAGGACTAGCATCCGCCAGG | 482 |
| 6507 | AGGACTAGCATCCGCCAGGG | 483 |
| 6508 | GGACTAGCATCCGCCAGGGG | 484 |
| 6509 | GACTAGCATCCGCCAGGGGG | 485 |
| 6510 | ACTAGCATCCGCCAGGGGGC | 486 |
| 6511 | CTAGCATCCGCCAGGGGGCG | 487 |
| 6512 | TAGCATCCGCCAGGGGGCGC | 488 |
| 6513 | AGCATCCGCCAGGGGGCGCG | 489 |
| 6514 | GCATCCGCCAGGGGGCGCGG | 490 |
| 6515 | CATCCGCCAGGGGGCGCGGC | 491 |
| 6516 | ATCCGCCAGGGGGCGCGGCG | 492 |
| 6517 | TCCGCCAGGGGGCGCGGCGA | 493 |
| 6518 | CCGCCAGGGGGCGCGGCGAG | 494 |
| 6519 | ACGCGCGCCCGCCTCACTCA | 441 |
| 6520 | CACGCGCGCCCGCCTCACTC | 440 |
| 6521 | CCACGCGCGCCCGCCTCACT | 439 |
| 6522 | CCCACGCGCGCCCGCCTCAC | 438 |
| 6523 | TCCCACGCGCGCCCGCCTCA | 437 |
| 6524 | CTCCCACGCGCGCCCGCCTC | 436 |
| 6525 | CCTCCCACGCGCGCCCGCCT | 435 |
| 6526 | CCCTCCCACGCGCGCCCGCC | 434 |
| 6527 | ACCCTCCCACGCGCGCCCGC | 433 |
| 6528 | CACCCTCCCACGCGCGCCCG | 432 |
| 6529 | ACACCCTCCCACGCGCGCCC | 431 |
| 6530 | GACACCCTCCCACGCGCGCC | 430 |
| 6531 | GGACACCCTCCCACGCGCGC | 429 |
| 6532 | GGGACACCCTCCCACGCGCG | 428 |
| 6533 | TGGGACACCCTCCCACGCGC | 427 |
| 6534 | TTGGGACACCCTCCCACGCG | 426 |
| 6535 | CTTGGGACACCCTCCCACGC | 425 |
| 6536 | CCTTGGGACACCCTCCCACG | 424 |
| 6537 | CGTCATTCTGTTGCCCTTTGTACCTCG | 1226 |
| 6538 | GCGTCATTCTGTTGCCCTTT | 1225 |
| 6539 | TGCGTCATTCTGTTGCCCTT | 1224 |
| 6540 | ATGCGTCATTCTGTTGCCCT | 1223 |
| 6541 | TATGCGTCATTCTGTTGCCC | 1222 |
| 6542 | GTATGCGTCATTCTGTTGCC | 1221 |
| 6543 | TGTATGCGTCATTCTGTTGC | 1220 |
| 6544 | GTGTATGCGTCATTCTGTTG | 1219 |
| 6545 | CGCCACGGGCGCATCCATCCCTCCTGGG | 4454 |
| 6546 | GCCACGGGCGCATCCATCCC | 4455 |
| 6547 | CCACGGGCGCATCCATCCCT | 4456 |
| 6548 | CACGGGCGCATCCATCCCTC | 4457 |
| 6549 | ACGGGCGCATCCATCCCTCC | 4458 |
| 6550 | CGGGCGCATCCATCCCTCCT | 4459 |
| 6551 | GGGCGCATCCATCCCTCCTG | 4460 |
| 6552 | GGCGCATCCATCCCTCCTGG | 4461 |
| 6553 | GCGCATCCATCCCTCCTGGG | 4462 |
| 6554 | CGCATCCATCCCTCCTGGGC | 4463 |
| 6555 | CCGCCACGGGCGCATCCATC | 4453 |
| 6556 | ACCGCCACGGGCGCATCCAT | 4452 |
| 6557 | CACCGCCACGGGCGCATCCA | 4451 |
| 6558 | TCACCGCCACGGGCGCATCC | 4450 |
| 6559 | CTCACCGCCACGGGCGCATC | 4449 |
| 6560 | GCTCACCGCCACGGGCGCAT | 4448 |
| 6561 | AGCTCACCGCCACGGGCGCA | 4447 |
| 6562 | GAGCTCACCGCCACGGGCGC | 4446 |
| 6563 | TGAGCTCACCGCCACGGGCG | 4445 |
| 6564 | CTGAGCTCACCGCCACGGGC | 4444 |
| 6565 | GCTGAGCTCACCGCCACGGG | 4443 |
| 6566 | AGCTGAGCTCACCGCCACGG | 4442 |
| 6567 | CAGCTGAGCTCACCGCCACG | 4441 |
| 6568 | GCAGCTGAGCTCACCGCCAC | 4440 |
| 6569 | CGCAGCTGAGCTCACCGCCA | 4439 |
| 6570 | GCGCAGCTGAGCTCACCGCC | 4438 |
| 6571 | AGCGCAGCTGAGCTCACCGC | 4437 |
| 6572 | CAGCGCAGCTGAGCTCACCG | 4436 |
| 6573 | GCAGCGCAGCTGAGCTCACC | 4435 |
| 6574 | GGCAGCGCAGCTGAGCTCAC | 4434 |
| 6575 | GGGCAGCGCAGCTGAGCTCA | 4433 |
| 6576 | TGGGCAGCGCAGCTGAGCTC | 4432 |
| 6577 | GTGGGCAGCGCAGCTGAGCT | 4431 |
| 6578 | GGTGGGCAGCGCAGCTGAGC | 4430 |
| 6579 | CACCGCCCTCTAGCCGCCTGCGGG | 0 |
| 6580 | TTGCGGCGACTTTGGTTGTTGCCCGCGACGGT | 34 |
| 6581 | TGCGGCGACTTTGGTTGTTG | 35 |
| 6582 | GCGGCGACTTTGGTTGTTGC | 36 |
| 6583 | CGGCGACTTTGGTTGTTGCC | 37 |
| 6584 | GGCGACTTTGGTTGTTGCCC | 38 |
| 6585 | GCGACTTTGGTTGTTGCCCG | 39 |
| 6586 | CGACTTTGGTTGTTGCCCGC | 40 |
| 6587 | GACTTTGGTTGTTGCCCGCG | 41 |
| 6588 | ACTTTGGTTGTTGCCCGCGA | 42 |
| 6589 | CTTTGGTTGTTGCCCGCGAC | 43 |
| 6590 | TTTGGTTGTTGCCCGCGACG | 44 |
| 6591 | TTGGTTGTTGCCCGCGACGG | 45 |
| 6592 | TGGTTGTTGCCCGCGACGGT | 46 |
| 6593 | GGTTGTTGCCCGCGACGGTG | 47 |
| 6594 | GTTGTTGCCCGCGACGGTGG | 48 |
| 6595 | TTGTTGCCCGCGACGGTGGG | 49 |
| 6596 | TGTTGCCCGCGACGGTGGGA | 50 |
| 6597 | GTTGCCCGCGACGGTGGGAC | 51 |
| 6598 | TTGCCCGCGACGGTGGGACG | 52 |
| 6599 | TGCCCGCGACGGTGGGACGG | 53 |
| 6600 | GCCCGCGACGGTGGGACGGG | 54 |
| 6601 | CCCGCGACGGTGGGACGGGA | 55 |
| 6602 | CCGCGACGGTGGGACGGGAC | 56 |
| 6603 | GTTGCGGCGACTTTGGTTGT | 33 |
| 6604 | AGTTGCGGCGACTTTGGTTG | 32 |
| 6605 | CAGTTGCGGCGACTTTGGTT | 31 |
| 6606 | GCAGTTGCGGCGACTTTGGT | 30 |
| 6607 | TGCAGTTGCGGCGACTTTGG | 29 |
| 6608 | CTGCAGTTGCGGCGACTTTG | 28 |
| 6609 | GCTGCAGTTGCGGCGACTTT | 27 |
| 6610 | TGCTGCAGTTGCGGCGACTT | 26 |
| 6611 | GTGCTGCAGTTGCGGCGACT | 25 |
| 6612 | TGTGCTGCAGTTGCGGCGAC | 24 |
| 6613 | CTGTGCTGCAGTTGCGGCGA | 23 |
| 6614 | TCTGTGCTGCAGTTGCGGCG | 22 |
| 6615 | CTCTGTGCTGCAGTTGCGGC | 21 |
| 6616 | GCTCTGTGCTGCAGTTGCGG | 20 |
| 6617 | CGCTCTGTGCTGCAGTTGCG | 19 |
| 6618 | CCGCTCTGTGCTGCAGTTGC | 18 |
| 6619 | CCCGCTCTGTGCTGCAGTTG | 17 |
| 6620 | GCCCGCTCTGTGCTGCAGTT | 16 |
| 6621 | TGCCCGCTCTGTGCTGCAGT | 15 |
| 6622 | TTGCCCGCTCTGTGCTGCAG | 14 |
| 6623 | TTTGCCCGCTCTGTGCTGCA | 13 |
| 6624 | CTTTGCCCGCTCTGTGCTGC | 12 |
| 6625 | GCTTTGCCCGCTCTGTGCTG | 11 |
| 6626 | GGCTTTGCCCGCTCTGTGCT | 10 |
| 6627 | TGGCTTTGCCCGCTCTGTGC | 9 |
| 6628 | CTGGCTTTGCCCGCTCTGTG | 8 |
| 6629 | CCTGGCTTTGCCCGCTCTGT | 7 |
| 6630 | GCCTGGCTTTGCCCGCTCTG | 6 |
| 6631 | TGCCTGGCTTTGCCCGCTCT | 5 |
| 6632 | CTGCCTGGCTTTGCCCGCTC | 4 |
| 6633 | CCTGCCTGGCTTTGCCCGCT | 3 |
| 6634 | GCCTGCCTGGCTTTGCCCGC | 2 |
| 6635 | GGCCTGCCTGGCTTTGCCCG | 1 |

Hot Zones (Relative upstream location to gene start site)
1-1000
1050-1450
1600-1900
3300-3800
4250-4700
4750-5000

### Examples

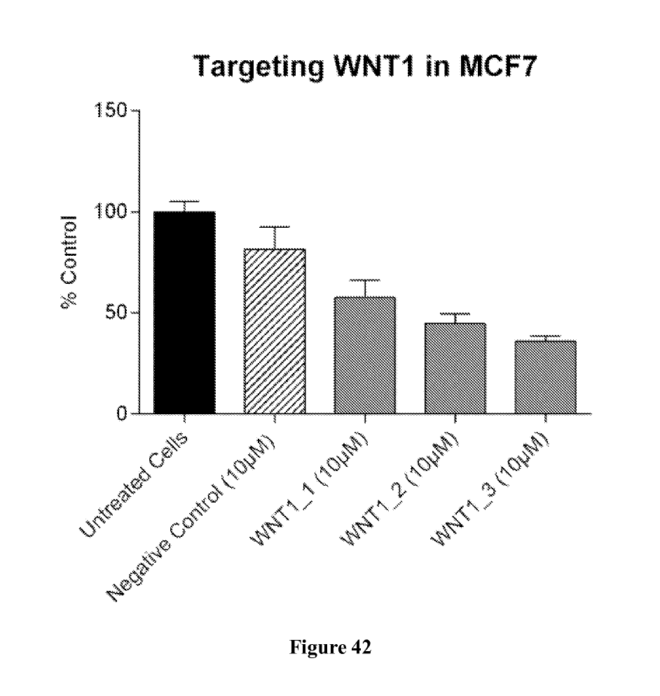

In FIG. 42, In MCF7 (human mammary breast cell line), WNT1_{—}1, WNT1_{—}2, WNT1_{—}3 produced statistically significant (P<0.05) inhibition at 10 μM compared to the untreated control values. The WNT1 sequences WNT1_{—}1, WNT1_{—}2, and WNT1_{—}3 fit the independent and dependent DNAi motif claims.

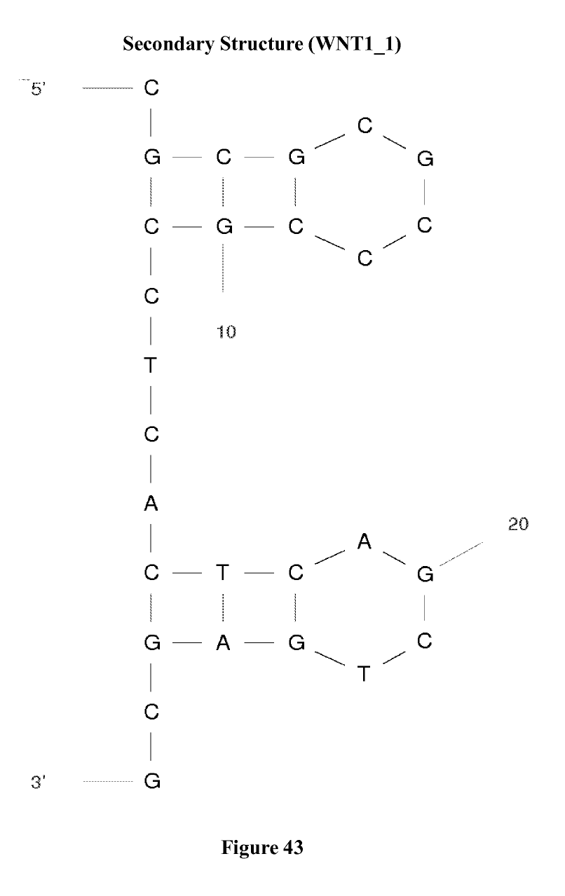

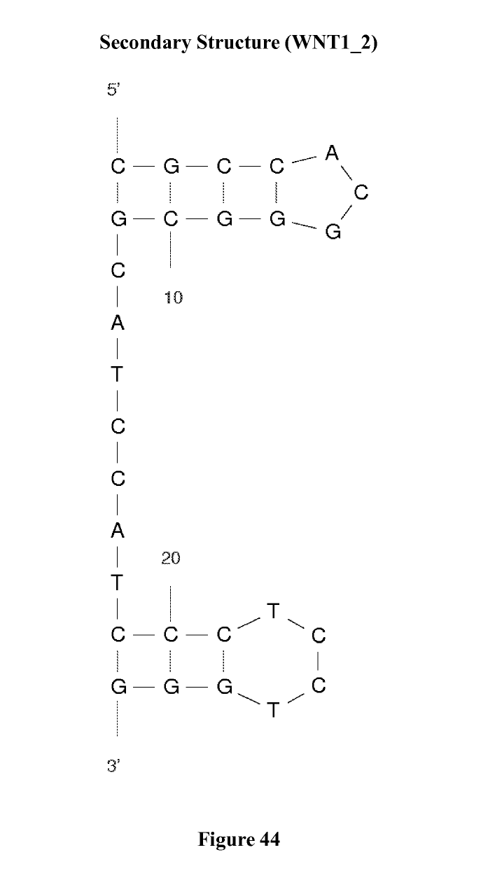

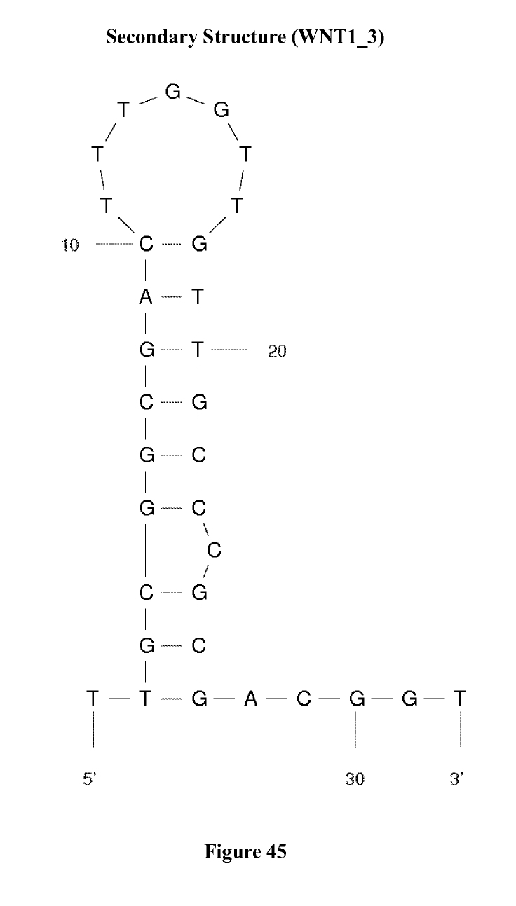

The secondary structures for WNT1_{—}1, WNT1_{—}2, and WNT1_{—}3 are shown in FIG. 43, FIG. 44, and FIG. 45.

Genetic Code (5′ Upstream Region)

(SEQ ID NO: 11972)

CCCGGGGAACCCAAATTATAGGCCCAGGAGGGATGGATGCGCCCGTGGCG
GTGAGCTCAGCTGCGCTGCCCACCCTCCGCTTAATGCGCCTTCTGCTGCA
GCACCGTAGGCCACCACCTGGAGGCACCAAAGGGTCTGCGGGCCGACTGC
ATACTGGACTCTCAGGAAGGCCCCACTTTCAGCAGTCCACTCCAACAAAT
CCATGGGATTACCTTAGACAGAATTTTGCCCCCTTCTGTACTCAGGCCTA
ATGGATGGGCTGTGCCTTCCCAGCCCAAGGGGGCAGTGCTGCCTGCGGGT
GCTTCAAAGGAGGGTAGGCTCCTCTGCCCACAAACTCTAAACCCTGGAGC
CCTGCTTCCTCCCCAGATCCCAAAGTCAAGGCAAAGCCCCTCTCCCCTCT
AACATCTCACCTCTAACCCTATTCCAGGGGGGTGGTTTGCTACTGATTTT
CAACTTCAAGCCTTTAAAGTCATCCACGGTCAAAACTGATACAGAGAAAA
ATGAAGCAGGGTAAAGGAGATTAGTAGTGGGATTCTATTTTATAAAGGGG
GAGGGAAAACAAACTGAAGGAACAAATACATGGAGAGATCTGAGGAAGAG
CATTTTAGAAGACAGAAAAGCAGGTGCACAGACCCTTAGAAAGGAGCATG
CTTGGTTCAAAGGATTAGAAAAGAGGCCAGTGAGGCTAGTGGGGAGAAAT
TCGCAAGGAAGAGAGTGGTAGGCAATGAAGCTGGAGGGCTAGGAAGGAGG
CCCCTTTACTTTGAGTGACATGGGGTCTCGCTGGAAAATTTTGAGCAGAG
AAATGAACTAATCAGACTTCTGTTTTAGGAAAGATGGCTCTGGGCTGGGC
GCAGTGGCTCATGCCTGTAATCCCAGTACTTTGGGAGGCCGACGTGGGCA
GATCACGAGGTCAGGAGTTCGAGACCAGCCTGGCCAACATGGTGAAACCC
CACCTCTACTAAAAATACAAAAATTAGGTGGGAGTGTTGGTGGGCGCCTG
TAATCCCAGCTACTCGGTACGCTGAGGCAGGAGAATCGCTTGAAACCGGA
AGGTGGAGGTTACAGTGAGCCGAGATCATACCACTGCACTCCAGTCTGCA
CAACAAGAGCAAAACTCCGTCTCAAAAAAAAAAAAAAAGAAAGAAAGAAA
GAAAGAAATTGGCTCTAGTAATTAAATCAACCCTTTTGATTTTTGCAGTA
AAATGGAGCACTGAAATGGAGCAATCTTGGGCAGTAATGTGGGGTCTAAT
GAAGTTTTTGGGTTTTTCAGATGGGTACTATTGCAGCATGTCTGCATGCT
TATGTGTATGTTCCAGGAGAGAGATAAGTGGATGATGCAGGAAAGAAAGA
GGGGACAGTTGATATCATGATTATTTGATCTAAATAGAAAGTTGGGTGCT
TGTTTTGGCAGCACATATACTAAAATTGGAATGATATAGAGATTAGCATG
GCCCCTGCACAAGGATGACATGCAAATTTGTGAAGCATTTCATATTTTGA
AAAAGAAATGTCAGCCAGGTCATAAACAGTGTGACCCAGATCTAGGGGCC
TTACCCTCTTGCCCCCTACTCCTGGTGTGTGGAATGTTGGAACAAAGCAC
AGTGGCTCCTTTCCTCTCTTCCCACCTCTGCTTGACAACAGTCGTCAAAG
ACAGGGCTTCCATATTTTCCAGCCAGCCTCCCACCCTCACGGTGTTGTAT
CAATCCACCAGGCCAAAAGATGTGACCCAGGCCCCAGTGGGAAGAAACTC
ATAAGGGATAAAGGACAGGCTCCCCGTGATACATTGTCCATTTACTTGAG
CTATCTATGCTGGGTGCTCTCTGCAGGGACTACTGGCTTTTGGATCTACG
GAGGGTGCTGGACCACTACACCTTTTCCCTCTGGGGTGGATCCTTGGAAG
GGCCAGATATACTAGGCTGGGCAAAAGGGAAGAAAAAAGGGAAAGAAGGA
CATTTCTTTCTAAAATAACTTCCATCAGGCTTCATTTGGGTTAATATGCA
TCTCATTTAAAACACAAGTGCCCGGGAATATTAATGAAACTTACCTGGCA
TTTATTCCTTAGAGTGATTTCCCTGCCTTAGAAGGGAATCCTAGTCATTT
CTGGGACTTGAGACTTTAGGTTCAGGCCTGGGGAAATTTCTCAGTCAGAA
GGCATCCTAAAAGACAAGGGAGATGAAAAAAATGAGAGCTAGAACTCAAA
AGGGAGGCAGAAAGGCCCAAAAAATTATTTTTACCCATCAATTTTGAGAA
GGGTTCCCAGCCTGTAATTGCTGCACACTGGCAAGCAGCTGGTAAGGTCG
AAAGAGCATGGGCTTTGAGTCAAATTGGTCTGGGTTTTAATCTGGCTCTA
CCATTGATTCATTTATTAGACGTGGACCTTGGACAAGTGCCTGATCTATT
TTTAATTCTGCAAAATGGGGAGAGAGAAGAGATCTTCCCTCCTTCCAGGG
GCCATGTGTGTGGTGGTGGGGCATGATAACCAGGCTGGCAGTGCCCCCTA
TTCCCCATATAGGGAAAAGCAGCCACTTTTTTTTTTTTTTTCAGATGAAG
TCTTGCTCTGTAGCCCAGACTGGAGTGCAATGGCGTGATCTCGGCTCACT
GCAATCTCCACCTCCCGGGTTCAAGCCATTCTCCTGCCTCAGCCTCCTGA
GTAGCTGGGACTACAGGTGTGCGCCACCACACTCAGCTAATTTTTGTATT
TTTATTAGAGATGGGATTCCACTATGTTGGCCAGGATGGTCTCGATCTCC
TGACCTCATGATCCACCTGCCTCGGCCTCCCAAAGTGCGGGGATTATAGG
CATGAGCCACCGCGCCCAGCCTGTCACTTCTTCAATAGGAGGCCTAAATG
GCCTTGAAGCTGAGTAGGAGTCCCTGGGAGAGAAGAGAAAAGTGTACAAT
GGATGAGATGGTCACAGGCACTCTGGGTATCCCAGTGTGGTGGGAACTAG
AGCTTTAGGGAAAGACAGAAACTTGGCAGAAACATCCAAAGAGAAGCAAA
CACATGGAGGCACAAGTTTCCTCATCTAGGTTCAATGTAGCCAGCAACCC
TTGTCTTCCCAGTCCTCTCCATCACCATACATACAGTGGACATCCGCACC
ATTTCCCATCCTTTCTGAGCCTAGGCCTCAGAGACTTAGCCACTCCAGGC
TGGGTTCACCTCAATACCATCTTGGTTGTAGGCTCGGCTCTCTCCCCCAA
TGACATGCACTGGTTGACACATACCACAGTGTGACACGCCATAGGATGCC
ACGAGGTACAAAGGGCAACAGAATGACGCATACACACATATTTAATCTTC
CCATGCACATGCTCATCCACCCACTCCACACACAGTCCAGACACTCTGCA
TCCCTCAATCATGCTTCTGAGTCTCCTGTCGACAGTTGCCACCTCCTTCC
TGACACACTGCCCCAGGCGGTGACTGTGACAAGGTGACTCCATGACCTTT
TCTGACTTGAGCTAAATTCCAAAATTCTTTGGAAAGTTTCCTAACATCCT
TCGTCAGAACAAGGAGTTTCTGCACGTACCAACACACAGGAGGATGCACC
CTCAGAACACAGCACATTCTCACTCCCACCCATATTCACGTTGTTCCACT
TCACACACACACACACACACACACACACACACAGCCACTTGTGCGCTTCT
TCTGGCGCACATGAGCAAACTGCCTGTTGCTTTAGGTTTCTCTCCACCGC
TAGGCTCCTTTTGGTTAGCTCACCCCCACAACTCATCCCCGGGATTTCCC
TGACCACAGCCGCACTCACGCCCCCGTCTCCCCTTTTTCCTTCTCTGTCC
AGCCATCGGGGGTTCCTGGGCGGTTAAGCATCTCCCCGGAGTCGCTGCCC
AGAACCACAGCTTTCCTTCCGACACTCAGGATGGGGGAGAGAGGGGACGT
CGGAGGGGCCCGGGGTGACGTCGAGGGGACAACCCCACCGCGGGCGGCGA
GGCGGGCTGGGCCCCTGGCGGGCTCTCCCCGCAGCACACTCTCGCCGCGC
CCCCTGGCGGATGCTAGTCCTCGACGGGCTCCGGACGCTCAGCTGAGTGA
GGCGGGCGCGCGTGGGAGGGTGTCCCAAGGGGAGGGGTCCGCGGCCAGTG
CAGGCCCGGAGGCGGGGGCCACCGGGCAGGGGGCGGGGGTGAGCCCCGAC
GGCCAACCCGTCAGCTCTCGGCTCAGACGGGCGGGAACCACAGCCCCGCT
CGCTGCCCATTGTCTGCGCCCCTAACCGGTGCGCCCTGGTGCCACAGTGC
GGCCCGGAGGGGCAGCCTCCTCCCGTCACTTCAGCCAGCGCCGCAACTAT
AAGAGGCGGTGCCGCCCGCCGTGGCCGCCTCAGCCCACCAGCCGGGACCG
CGAGCCATGCTGTCCGCCGCCCGCCCCCAGGGTTGTTAAAGCCAGACTGC
GAACTCTCGCCACTGCCGCCACCGCCGCGTCCCGTCCCACCGTCGCGGGC
AACAACCAAAGTCGCCGCAACTGCAGCACAGAGCGGGCAAAGCCAGGCAG
GCCATG

24) Clusterin. Clusterin is a heterodimeric glycoprotein produced by a wide array of tissues and found in most biologic fluids. A number of physiologic functions have been proposed for clusterin based on its distribution and in vitro properties. These include complement regulation, lipid transport, sperm maturation, initiation of apoptosis, endocrine secretion, membrane protection, and promotion of cell interactions. A prominent and defining feature of clusterin is its induction in such disease states as glomerulonephritis, polycystic kidney disease, renal tubular injury, neurodegenerative conditions including Alzheimer's disease, atherosclerosis, and myocardial infarction (reviewed by Rosenberg and Silkensen, Int. J. Biochem Cell Biol. 1995: 27 (7) 633-645. Genome-wide association studies found a statistical association between a SNP within the clusterin gene and the risk of having Alzheimer's disease (Lambert et al., 2009: Nat. Genet. 41 (10): 1094-1099). Other studies, Alzheimer's patients have more clusterin in their blood (Schrijvers et al. 2011 JAMA 305 (13): 1322-1326).

Clusterin acts as cell-survival protein and is over-expressed in response to anti-cancer agents. An antisense approach to inhibiting clusterin (Curtisen) has shown promising results in combination with currently available chemotherapies in several tumor types. The FDA granted Custirsen two Fast Track Designations as a treatment in combination with first-line and second-line docetaxel for progressive metastatic prostate cancer.

Protein: Clusterin Gene: CLU (Homo sapiens, chromosome 8, 27454434-27472328 [NCBI Reference Sequence: NC_{—}000008.10]; start site location: 27468088; strand: negative)

Gene Identification

| | |
|---|---|
| GeneID | 1191 |
| HGNC | 2095 |
| HPRD | — |
| MIM | 185430 |

Targeted Sequences

| | | Relative |
|---|---|---|
| | | upstream |
| | | location |
| Sequence | | to gene |
| ID No: | Sequence (5′-3′) | start site |
| 6636 | CGTCCCGCCCACCTGCTGCCTGCAGCAG | 78 |
| 6660 | CGACAATCAGCGAGGCACACAGGCT | 330 |
| 6689 | CGGAGAGTAGAGAGGGTTCGCAGTGGCCC | 718 |
| 6690 | CCACGGGGCACAGGCCATAGCCCCG | 890 |
| 6709 | CTCGTGCTCTCAGGCGGCGGTTGCGCCG | 3865 |
| 6752 | CCGGGAGGTGGGGGCCGGTGCAGCACCGG | 4260 |
| 6753 | TCGCGTGCCCATCTGGGAGCCCCTCTCACG | 4395 |

Target Shift Sequences

| | | Relative |
|---|---|---|
| | | upstream |
| | | location |
| Sequence | | to gene |
| ID No: | Sequence (5′-3′) | start site |
| 6636 | CGTCCCGCCCACCTGCTGCCTGCAGCAG | 78 |
| 6637 | GTCCCGCCCACCTGCTGCCT | 79 |
| 6638 | TCCCGCCCACCTGCTGCCTG | 80 |
| 6639 | CCCGCCCACCTGCTGCCTGC | 81 |
| 6640 | CCGCCCACCTGCTGCCTGCA | 82 |
| 6641 | CGCCCACCTGCTGCCTGCAG | 83 |
| 6642 | GCGTCCCGCCCACCTGCTGC | 77 |
| 6643 | GGCGTCCCGCCCACCTGCTG | 76 |
| 6644 | TGGCGTCCCGCCCACCTGCT | 75 |
| 6645 | CTGGCGTCCCGCCCACCTGC | 74 |
| 6646 | GCTGGCGTCCCGCCCACCTG | 73 |
| 6647 | TGCTGGCGTCCCGCCCACCT | 72 |
| 6648 | CTGCTGGCGTCCCGCCCACC | 71 |
| 6649 | CCTGCTGGCGTCCCGCCCAC | 70 |
| 6650 | GCCTGCTGGCGTCCCGCCCA | 69 |
| 6651 | AGCCTGCTGGCGTCCCGCCC | 68 |
| 6652 | CAGCCTGCTGGCGTCCCGCC | 67 |
| 6653 | ACAGCCTGCTGGCGTCCCGC | 66 |
| 6654 | GACAGCCTGCTGGCGTCCCG | 65 |
| 6655 | AGACAGCCTGCTGGCGTCCC | 64 |
| 6656 | TAGACAGCCTGCTGGCGTCC | 63 |
| 6657 | CTAGACAGCCTGCTGGCGTC | 62 |
| 6658 | GCTAGACAGCCTGCTGGCGT | 61 |
| 6659 | AGCTAGACAGCCTGCTGGCG | 60 |
| 6660 | CGACAATCAGCGAGGCACACAGGCT | 330 |
| 6661 | GACAATCAGCGAGGCACACA | 331 |
| 6662 | ACAATCAGCGAGGCACACAG | 332 |
| 6663 | CAATCAGCGAGGCACACAGG | 333 |
| 6664 | AATCAGCGAGGCACACAGGC | 334 |
| 6665 | ATCAGCGAGGCACACAGGCT | 335 |
| 6666 | TCAGCGAGGCACACAGGCTT | 336 |
| 6667 | CAGCGAGGCACACAGGCTTT | 337 |
| 6668 | AGCGAGGCACACAGGCTTTC | 338 |
| 6669 | GCGAGGCACACAGGCTTTCT | 339 |
| 6670 | CGAGGCACACAGGCTTTCTG | 340 |
| 6671 | CCGACAATCAGCGAGGCACA | 329 |
| 6672 | CCCGACAATCAGCGAGGCAC | 328 |
| 6673 | CCCCGACAATCAGCGAGGCA | 327 |
| 6674 | TCCCCGACAATCAGCGAGGC | 326 |
| 6675 | CTCCCCGACAATCAGCGAGG | 325 |
| 6676 | CCTCCCCGACAATCAGCGAG | 324 |
| 6677 | TCCTCCCCGACAATCAGCGA | 323 |
| 6678 | ATCCTCCCCGACAATCAGCG | 322 |
| 6679 | CATCCTCCCCGACAATCAGC | 321 |
| 6680 | ACATCCTCCCCGACAATCAG | 320 |
| 6681 | CACATCCTCCCCGACAATCA | 319 |
| 6682 | CCACATCCTCCCCGACAATC | 318 |
| 6683 | GCCACATCCTCCCCGACAAT | 317 |
| 6684 | AGCCACATCCTCCCCGACAA | 316 |
| 6685 | AAGCCACATCCTCCCCGACA | 315 |
| 6686 | CAAGCCACATCCTCCCCGAC | 314 |
| 6687 | CCAAGCCACATCCTCCCCGA | 313 |
| 6688 | TCCAAGCCACATCCTCCCCG | 312 |
| 6689 | CGGAGAGTAGAGAGGGTTCGCAGTGGCCC | 718 |
| 6690 | CCACGGGGCACAGGCCATAGCCCCG | 890 |
| 6691 | CACGGGGCACAGGCCATAGC | 891 |
| 6692 | ACGGGGCACAGGCCATAGCC | 892 |
| 6693 | CGGGGCACAGGCCATAGCCC | 893 |
| 6694 | GCCACGGGGCACAGGCCATA | 889 |
| 6695 | AGCCACGGGGCACAGGCCAT | 888 |
| 6696 | GAGCCACGGGGCACAGGCCA | 887 |
| 6697 | TGAGCCACGGGGCACAGGCC | 886 |
| 6698 | CTGAGCCACGGGGCACAGGC | 885 |
| 6699 | CCTGAGCCACGGGGCACAGG | 884 |
| 6700 | CCCTGAGCCACGGGGCACAG | 883 |
| 6701 | GCCCTGAGCCACGGGGCACA | 882 |
| 6702 | TGCCCTGAGCCACGGGGCAC | 881 |
| 6703 | CTGCCCTGAGCCACGGGGCA | 880 |
| 6704 | GCTGCCCTGAGCCACGGGGC | 879 |
| 6705 | GGCTGCCCTGAGCCACGGGG | 878 |
| 6706 | TGGCTGCCCTGAGCCACGGG | 877 |
| 6707 | CTGGCTGCCCTGAGCCACGG | 876 |
| 6708 | GCTGGCTGCCCTGAGCCACG | 875 |
| 6709 | CTCGTGCTCTCAGGCGGCGGTTGCGCCG | 3865 |
| 6710 | TCGTGCTCTCAGGCGGCGGT | 3866 |
| 6711 | CGTGCTCTCAGGCGGCGGTT | 3867 |
| 6712 | GTGCTCTCAGGCGGCGGTTG | 3868 |
| 6713 | TGCTCTCAGGCGGCGGTTGC | 3869 |
| 6714 | GCTCTCAGGCGGCGGTTGCG | 3870 |
| 6715 | CTCTCAGGCGGCGGTTGCGC | 3871 |
| 6716 | TCTCAGGCGGCGGTTGCGCC | 3872 |
| 6717 | CTCAGGCGGCGGTTGCGCCG | 3873 |
| 6718 | TCAGGCGGCGGTTGCGCCGG | 3874 |
| 6719 | CAGGCGGCGGTTGCGCCGGG | 3875 |
| 6720 | AGGCGGCGGTTGCGCCGGGG | 3876 |
| 6721 | GGCGGCGGTTGCGCCGGGGC | 3877 |
| 6722 | GCGGCGGTTGCGCCGGGGCC | 3878 |
| 6723 | CGGCGGTTGCGCCGGGGCCC | 3879 |
| 6724 | GGCGGTTGCGCCGGGGCCCC | 3880 |
| 6725 | GCGGTTGCGCCGGGGCCCCT | 3881 |
| 6726 | CGGTTGCGCCGGGGCCCCTG | 3882 |
| 6727 | GGTTGCGCCGGGGCCCCTGG | 3883 |
| 6728 | GTTGCGCCGGGGCCCCTGGC | 3884 |
| 6729 | TTGCGCCGGGGCCCCTGGCT | 3885 |
| 6730 | TGCGCCGGGGCCCCTGGCTC | 3886 |
| 6731 | GCGCCGGGGCCCCTGGCTCA | 3887 |
| 6732 | CGCCGGGGCCCCTGGCTCAG | 3888 |
| 6733 | GCCGGGGCCCCTGGCTCAGC | 3889 |
| 6734 | CCGGGGCCCCTGGCTCAGCT | 3890 |
| 6735 | CGGGGCCCCTGGCTCAGCTG | 3891 |
| 6736 | GCTCGTGCTCTCAGGCGGCG | 3864 |
| 6737 | AGCTCGTGCTCTCAGGCGGC | 3863 |
| 6738 | GAGCTCGTGCTCTCAGGCGG | 3862 |
| 6739 | GGAGCTCGTGCTCTCAGGCG | 3861 |
| 6740 | TGGAGCTCGTGCTCTCAGGC | 3860 |
| 6741 | TTGGAGCTCGTGCTCTCAGG | 3859 |
| 6742 | GTTGGAGCTCGTGCTCTCAG | 3858 |
| 6743 | GGTTGGAGCTCGTGCTCTCA | 3857 |
| 6744 | TGGTTGGAGCTCGTGCTCTC | 3856 |
| 6745 | GTGGTTGGAGCTCGTGCTCT | 3855 |
| 6746 | TGTGGTTGGAGCTCGTGCTC | 3854 |
| 6747 | TTGTGGTTGGAGCTCGTGCT | 3853 |
| 6748 | ATTGTGGTTGGAGCTCGTGC | 3852 |
| 6749 | AATTGTGGTTGGAGCTCGTG | 3851 |
| 6750 | GAATTGTGGTTGGAGCTCGT | 3850 |
| 6751 | AGAATTGTGGTTGGAGCTCG | 3849 |
| 6752 | CCGGGAGGTGGGGGCCGGTGCAGCACCGG | 4260 |
| 6753 | TCGCGTGCCCATCTGGGAGCCCCTCTCACG | 4395 |
| 6754 | CGCGTGCCCATCTGGGAGCC | 4396 |
| 6755 | GCGTGCCCATCTGGGAGCCC | 4397 |
| 6756 | CGTGCCCATCTGGGAGCCCC | 4398 |
| 6757 | CTCGCGTGCCCATCTGGGAG | 4394 |
| 6758 | ACTCGCGTGCCCATCTGGGA | 4393 |
| 6759 | AACTCGCGTGCCCATCTGGG | 4392 |
| 6760 | GAACTCGCGTGCCCATCTGG | 4391 |
| 6761 | TGAACTCGCGTGCCCATCTG | 4390 |
| 6762 | CTGAACTCGCGTGCCCATCT | 4389 |
| 6763 | CCTGAACTCGCGTGCCCATC | 4388 |
| 6764 | GCCTGAACTCGCGTGCCCAT | 4387 |
| 6765 | AGCCTGAACTCGCGTGCCCA | 4386 |
| 6766 | GAGCCTGAACTCGCGTGCCC | 4385 |
| 6767 | AGAGCCTGAACTCGCGTGCC | 4384 |
| 6768 | AAGAGCCTGAACTCGCGTGC | 4383 |
| 6769 | GAAGAGCCTGAACTCGCGTG | 4382 |
| 6770 | GGAAGAGCCTGAACTCGCGT | 4381 |
| 6771 | GGGAAGAGCCTGAACTCGCG | 4380 |
| 6772 | AGGGAAGAGCCTGAACTCGC | 4379 |
| 6773 | TAGGGAAGAGCCTGAACTCG | 4378 |

Hot Zones (Relative upstream location to gene start site)
1-950
1000-1300
2050-3000
3550-4500

### Examples

Genetic Code (5′ Upstream Region)

(SEQ ID NO: 11973)

AATGTGAAGGTTAAGGTCAGTAGGGCCAGGGAACTGTGAGATTGTGTCTT
GGACTGGGACAGACAGCCGGGCTAACCGCGTGAGAGGGGCTCCCAGATGG
GCACGCGAGTTCAGGCTCTTCCCTACTGGAAGCGCCGAGCGGCCGCACCT
CAGGGTCTCTCCTGGAGCCAGCACAGCTATTCGTGGTGATGATGCGCCCC
CCGGCGCCCCCAGCCCGGTGCTGCACCGGCCCCCACCTCCCGGCTTCCAG
AAAGCTCCCCTTGCTTTCCGCGGCATTCTTTGGGCGTGAGTCATGCAGGT
TTGCAGCCAGCCCCAAAGGGGGTGTGTGCGCGAGCAGAGCGCTATAAATA
CGGCGCCTCCCAGTGCCCACAACGCGGCGTCGCCAGGAGGAGCGCGCGGG
CACAGGGTGCCGCTGACCGGTGAGATGTCCCCGTCTTCCCTACCCTTGAG
CAGAGCCACACCAGGACGGATGGGCGGGCAGGGGATGGCAGCCAGGCAGA
GAGGGATGACACAGCTCGCAGTCACAACCCCTGCGCTTTCGACGGAGCCC
AGGAAGCCAGGGAGGGGAGGTGGCCGGAGCCCCATCACCAGGCAGCTGAG
CCAGGGGCCCCGGCGCAACCGCCGCCTGAGAGCACGAGCTCCAACCACAA
TTCTGTGGTGGGGGGGTAAATAGAACAGATATAATGATCATCCTTTCGCA
AAGATGGGGAAACTGAGACCTGGAGACCTGCCGCGTTGCGGGAGACCCAG
GCTAGCAGGTGACAGAGCTGGCCTGCACCGAGCTCCTTCCTGCAGCATAT
CCTCTGCGAAGATGCGGATCTCTCAGTTGTGGCTTTCGGCTTGCATGCAT
GAGTCATCTAGTTTTCTTCTAAATTCTCTAGCTCTCTGGACACTGTTGCC
TGTAAGTATGAGGCTGCGGATTTCAGTATATGCTGCAACCACCGAAATCC
GACTTTTTCTGCCTCCTAATGCATCTGAGGTGCATCAGAGAAAAGTCACA
CAAGATCCACCAGGCCTCAGACCTCTGATTCCACAGTCTCATTTTACAGA
TGATAATCTGAGGCCTGGAGAGGTTTAGGACTGGTGCCAACACTAAACAG
CAAATAAGTATCAGAATTGGGATTCGAGCCAAAGCCTCTTGACCTTCCAG
AATTTCTGGACCTAGTTAAAAAAAATATGATTTTTATTATTATTTTTTAA
ACGGAGAGGTTAGGAATTTAAAGGAAAGTACAGATACTATATAAAAAAAG
ATGCCCATGAAAATGTTAAGTTATAATAATAGTGGAGCATTGGGCACAAC
TGAAATGGCCAATCTTGTGAGAATGGTAAAATAAACTTAGGTCCGTGAGT
AAGTGGAGTATTACATAGCCATAAAAGTATGCCCTTAAAGAATATTTGAA
GATGGTGAATGTGAAGAATCTTGTATAAACTGCATGGAAGACAGAAGGAA
ATATACCACAGTGCTAACCTTTGCCTCTGGGTGATATGAATTACCGGTGA
TTATTTTTCTTATTTTCCTTTTGGTTTAGTTTTCTCCATTTGAAGAAGCA
GATAGGAGCCGGGGCTTTGGGATTGAAACCCTCACCATCTGTGTGCCCTC
TTCACTGTCTTCCCATCCTCCCCACGGCTCCCTGTTCACAGTCATTGATT
TTCTTTCTTTCTTTTCTCTCTTTTTTTTTTTTTTTCCTGAGACCAAGTCT
CACTCTGTTGCCCAGGCTGGAGTAGAGTAGCGCCATCTCGGCTCACTGCA
ACCTCCGCCATCCGGGTTCAAGCAGTTCTCATGCCTCAGCCTCTGAGTAG
CTGGGACTACAGGCGCATGCTGCTACATCCGGCTAATTTTTGTATTTTTA
GTAGAGACATGGTTTCACCACCTTGGCCAGGCTGGTCTCGAACTCCTGAT
CTCAAGTAATCCGCCTGTCTTGGCCTCCCAAAGTGCTGGGGTGACAGGTG
TGAATCAATGCGCCCTGCCAGGTCATTGATTTTCTTAAGCCTCCAGCCCT
GCCCTGCTTGGAAACGTTTTGGGAAGCTGCTCAGTTCAAAGTTCCCAGGA
GGGTGTGCCTGGAGGGGAGTTGCTCCCAAAGTCTGCCTGCTCCCCCCGCC
CCCCCTGCCCCCCACCCCCCGCCATCTTCTCCTCCTCCTCTTCCCCTGAG
CAGCCCCTTTGTCCACAGAACCGGCCTTTTCTGGTAGAAGGAGCAAGGCC
AAGTGGTTTAAGCCTTCTTAGGGAGAATGAGGCTGTGTGGTAGTGCTGGG
GACTCGAGGGCCTTGGCCTTGGCATGGCTCTTCCACCCAGGGCAGCTGGC
AGCCAGGCTCCCAGGAGGCAGAGGAGATGAGGGGGGAGGTGAGTCCGAGC
AAAGGAAAGGAGGTCGGCTGTGCAGTCACGGTTCTAGAACATTCCTTGGA
TCAGCAGCATCCATATCACCTGCAGACTGGCTGGAAAAGCAGTCTCAGAA
CCAACATTATAACCAGCCCTGCAGTGATTCATAAGTACTTTAAAAAGTGG
TCAATCATTTCAGCAAAGCAGAGCCACACAGTCCGGGGGACCACAGGTGG
CCTCTGTGTGCTTGTCTCGGTTTTCCTGCCCCTCTCCAGACATGTTGATT
AGACACTGCCAATGCCCAGCCTCAGACCTCAGTCTAATTTGGAAGTAGTC
AGAATTTACTATGATTACATAAGACCCTCGTGTTTACAGAACACATTCCC
CTCTCTGAGGTCTGGATTAGATCCATTTTACAGATGAAGAAACTGAGGCT
CAGATATTTAAGTGACTTGGAATCAAGGAAAGAATACTGGACTAGGGGTC
GGGAGGGCTGGGCTCTCATCCCAGGGTTACCATGAGCATGCTGTGGACTC
TAGGGAGTCCCATGCCCTCTCTGGGCTTCAGCCTCACCGCTAGGGTAGAG
AGGTTGGGTGAGAGAACGACCTCCTTCCCAGGTCTGAGCTGGATGGTTCA
CCAGGGACCCCAGGCTCCCTGGAGCAGACTCTGTGCCCGCTGCTGAGTCT
GGAATTCCTTTCCTGTATCTTGCCTTTGGCTGCCCCATTCTTCATGGCCC
AGCACCCTGTCTTCTGGTCAGAACCTAGTTCTGAATGGGTTTTTCCAGAA
GTTGTTGCTTTCAGGGGCCCCTGGCAGAGAGGTGTTTCTGGCTGGCTTTG
TCTCTCTGGCATGACAAAGGCTCTGTTCCTGCTGGAGGCATTTCAGGGCT
CAGTGGGCAGCTGGGGCAGAGCCCGTGAGACCACAGCCTTCCTGGTGAGC
CCGGTCTCCGCCCCCTACCCCATCTCTGGGGAAGGCGCTGACCCCATCTC
TTCTCCCACGCTGCTCCCTGGCTCTTTGCGCCTGATTACTTCTCATGAGA
GGCACTCCTTGTTAATGTGCTACTGAGTGTCCAGATGGGCCTGCTGGGCT
GAGCGGGCTTTGGATGTGAACCATTTCAGGAAGGGGAACCCCATCGTCCT
GTTGGTTCTGTGATGGCAAATGGGTGAGCTCAGATAAGCAGTTCTTGGGA
GGGGCATGGTGGGGGTGGAGTGCAGGGGGAGGGGTTTCTGTTTTATGCAA
CAGCCTCAGCTTCTGGGAAAGGGTCCATTGTGTAAGACCGGGGCTATGGC
CTGTGCCCCGTGGCTCAGGGCAGCCAGCCCAGTGGTGGCAGGAACACTGG
CAGGGCAGCCTGCTGTCGGCTTAGAGGGGATGGGCAGTGTGGAGGGCCTG
GCAGAGCAAGAGGACTCATCCTTCCAAAGGGACTTTCTCTGGGAAGCCTG
CTCCTCGGGCCACTGCGAACCCTCTCTACTCTCCGAAGGGAATTGTCCTT
CCTGGCTTCCACTACTTCCACCCCTGAATGCACAGGCAGCCCGGCCCAAG
TCTCCCACTAGGGATGCAGATGGATTCGGTGTGAAGGGCTGGCTGCTGTT
GCCTCCGGCTCTTGAAAGTCAAGTTCAGGTGGTGCTGAGACTCCCTGGGG
GCTGCAGCGCTGTGGTGAATGGGGAGCGTCTGCTGGGGTGAAGGTTTAGG
TGCACATTGCAGAGGACGTGGCTGGTCTCTGGGATGCAGTCCCTCTGTGG
AGGTGGCATGGGGAGGGACGGATGCATGACCTAAGGGGGGTATTTTCAGT
GTCTGACATGATCGATACCACTCTGGACAAGGAGGCCAGGATGCAGAAAG
CCTGTGTGCCTCGCTGATTGTCGGGGAGGATGTGGCTTGGACAAGAGCCT
GGTTCCTCCGATGCCAGGGTTCTTGTTTCTTCCACTCAACATTGCTGTCC
TGCAGTCCCTCCCTCCCTGCACCTCCTGCCTTCGCTTTCATTCGAGGTGT
CCATGGCAAGTCTGGTCATTTCCCCCCATTTCCTCAGGAATAAAAGTGCA
GCAGTGCCTGCTGTGGGGACAGCTGAGGGCAGTGAGGCCCTGGGGAGCTG
CTGCAGGCAGCAGGTGGGCGGGACGCCAGCAGGCTGTCTAGCTGTTCCCA
TGATGGTCTCCTGTTCTCTGCAGAGGCGTGCAAAGACTCCAGAATTGGAG
GCATG

25. NRAS. The neuroblastoma RAS viral oncogene homolog (N-ras) oncogene is a member of the Ras gene family. It is mapped on chromosome 1, and it is activated in HL60, a promyelocytic leukemia line. The mammalian ras gene family consists of the harvey and kirsten ras genes (HRAS and KRAS), an inactive pseudogene of each (c-Hras2 and c-Kras1) and the N-ras gene. They differ significantly only in the C-terminal 40 amino acids. These ras genes have GTP/GDP binding and GTPase activity, and their normal function may be as G-like regulatory proteins involved in the normal control of cell growth. Mutations which change amino acid residues 12, 13 or 61 activate the potential of N-ras to transform cultured cells and are implicated in a variety of human tumors. The N-ras gene specifies two main transcripts of 2 Kb and 4.3 Kb. The difference between the two transcripts is a simple extension through the termination site of the 2 Kb transcript. The N-ras gene consists of seven exons (-I, I, II, III, IV, V, VI). The smaller 2 Kb transcript contains the VIa exon, and the larger 4.3 Kb transcript contains the VIb exon which is just a longer form of the VIa exon. Both transcripts encode identical proteins as they differ only the 3′ untranslated region (reviewed in Marshall et al., 1982 Nature 299 (5879): 171-3 and Shimizu et al., 1983 PNAS 80 (2): 383-7).

Protein: NRAS Gene: NRAS (Homo sapiens, chromosome 1, 115247085-115259515 [NCBI Reference Sequence: NC_{—}000001.10]; start site location: 115258781; strand: negative)

Gene Identification

| | |
|---|---|
| GeneID | 4893 |
| HGNC | 7989 |
| HPRD | 01273 |
| MIM | 164790 |

Targeted Sequences

| | | Relative |
|---|---|---|
| | | upstream |
| | | location to |
| Sequence | | gene |
| ID No: | Sequence (5′-3′) | start site |
| 6774 | CCCCGCCCTCAGCCTAAGCAATGGA | 234 |
| 6793 | GACCCCGGAACCGCCATGAACAGCCC | 559 |
| 6818 | CCCGCTACGTAATCAGTCGGCGCCCCA | 613 |
| 6961 | AACGCAAAAACACCGGATTAATATCGGCCT | 142 |
| 6963 | ATAAACGGCCTCTTTACCCAGAGATCA | 850 |
| 6971 | CGCCACCTTAAGTTTTTCCAGGCTGC | 1779 |

Target Shift Sequences

| | | Relative |
|---|---|---|
| | | upstream |
| | | location to |
| Sequence | | gene |
| ID No: | Sequence (5′-3′) | start site |
| 6774 | CCCCGCCCTCAGCCTAAGCAATGGA | 234 |
| 6775 | CCCGCCCTCAGCCTAAGCAA | 235 |
| 6776 | CCGCCCTCAGCCTAAGCAAT | 236 |
| 6777 | CGCCCTCAGCCTAAGCAATG | 237 |
| 6778 | GCCCCGCCCTCAGCCTAAGC | 233 |
| 6779 | GGCCCCGCCCTCAGCCTAAG | 232 |
| 6780 | GGGCCCCGCCCTCAGCCTAA | 231 |
| 6781 | TGGGCCCCGCCCTCAGCCTA | 230 |
| 6782 | TTGGGCCCCGCCCTCAGCCT | 229 |
| 6783 | CTTGGGCCCCGCCCTCAGCC | 228 |
| 6784 | CCTTGGGCCCCGCCCTCAGC | 227 |
| 6785 | TCCTTGGGCCCCGCCCTCAG | 226 |
| 6786 | GTCCTTGGGCCCCGCCCTCA | 225 |
| 6787 | AGTCCTTGGGCCCCGCCCTC | 224 |
| 6788 | CAGTCCTTGGGCCCCGCCCT | 223 |
| 6789 | ACAGTCCTTGGGCCCCGCCC | 222 |
| 6790 | AACAGTCCTTGGGCCCCGCC | 221 |
| 6791 | CAACAGTCCTTGGGCCCCGC | 220 |
| 6792 | TCAACAGTCCTTGGGCCCCG | 219 |
| 6793 | GACCCCGGAACCGCCATGAACAGCCC | 559 |
| 6794 | ACCCCGGAACCGCCATGAAC | 560 |
| 6795 | CCCCGGAACCGCCATGAACA | 561 |
| 6796 | CCCGGAACCGCCATGAACAG | 562 |
| 6797 | CCGGAACCGCCATGAACAGC | 563 |
| 6798 | CGGAACCGCCATGAACAGCC | 564 |
| 6799 | GGAACCGCCATGAACAGCCC | 565 |
| 6800 | GAACCGCCATGAACAGCCCC | 566 |
| 6801 | AACCGCCATGAACAGCCCCC | 567 |
| 6802 | ACCGCCATGAACAGCCCCCA | 568 |
| 6803 | CCGCCATGAACAGCCCCCAC | 569 |
| 6804 | CGCCATGAACAGCCCCCACC | 570 |
| 6805 | AGACCCCGGAACCGCCATGA | 558 |
| 6806 | GAGACCCCGGAACCGCCATG | 557 |
| 6807 | GGAGACCCCGGAACCGCCAT | 556 |
| 6808 | TGGAGACCCCGGAACCGCCA | 555 |
| 6809 | TTGGAGACCCCGGAACCGCC | 554 |
| 6810 | GTTGGAGACCCCGGAACCGC | 553 |
| 6811 | TGTTGGAGACCCCGGAACCG | 552 |
| 6812 | ATGTTGGAGACCCCGGAACC | 551 |
| 6813 | AATGTTGGAGACCCCGGAAC | 550 |
| 6814 | AAATGTTGGAGACCCCGGAA | 549 |
| 6815 | AAAATGTTGGAGACCCCGGA | 548 |
| 6816 | AAAAATGTTGGAGACCCCGG | 547 |
| 6817 | GAAAAATGTTGGAGACCCCG | 546 |
| 6818 | CCCGCTACGTAATCAGTCGGCGCCCCA | 613 |
| 6819 | CCGCTACGTAATCAGTCGGC | 614 |
| 6820 | CGCTACGTAATCAGTCGGCG | 615 |
| 6821 | GCTACGTAATCAGTCGGCGC | 616 |
| 6822 | CTACGTAATCAGTCGGCGCC | 617 |
| 6823 | TACGTAATCAGTCGGCGCCC | 618 |
| 6824 | ACGTAATCAGTCGGCGCCCC | 619 |
| 6825 | CGTAATCAGTCGGCGCCCCA | 620 |
| 6826 | GTAATCAGTCGGCGCCCCAG | 621 |
| 6827 | TAATCAGTCGGCGCCCCAGG | 622 |
| 6828 | AATCAGTCGGCGCCCCAGGC | 623 |
| 6829 | ATCAGTCGGCGCCCCAGGCG | 624 |
| 6830 | TCAGTCGGCGCCCCAGGCGC | 625 |
| 6831 | CAGTCGGCGCCCCAGGCGCC | 626 |
| 6832 | AGTCGGCGCCCCAGGCGCCT | 627 |
| 6833 | GTCGGCGCCCCAGGCGCCTG | 628 |
| 6834 | TCGGCGCCCCAGGCGCCTGA | 629 |
| 6835 | CGGCGCCCCAGGCGCCTGAG | 630 |
| 6836 | GGCGCCCCAGGCGCCTGAGT | 631 |
| 6837 | GCGCCCCAGGCGCCTGAGTC | 632 |
| 6838 | CGCCCCAGGCGCCTGAGTCC | 633 |
| 6839 | GCCCCAGGCGCCTGAGTCCC | 634 |
| 6840 | CCCCAGGCGCCTGAGTCCCC | 635 |
| 6841 | CCCAGGCGCCTGAGTCCCCG | 636 |
| 6842 | CCAGGCGCCTGAGTCCCCGC | 637 |
| 6843 | CAGGCGCCTGAGTCCCCGCC | 638 |
| 6844 | AGGCGCCTGAGTCCCCGCCC | 639 |
| 6845 | GGCGCCTGAGTCCCCGCCCC | 640 |
| 6846 | GCGCCTGAGTCCCCGCCCCG | 641 |
| 6847 | CGCCTGAGTCCCCGCCCCGG | 642 |
| 6848 | GCCTGAGTCCCCGCCCCGGC | 643 |
| 6849 | CCTGAGTCCCCGCCCCGGCC | 644 |
| 6850 | CTGAGTCCCCGCCCCGGCCA | 645 |
| 6851 | TGAGTCCCCGCCCCGGCCAC | 646 |
| 6852 | GAGTCCCCGCCCCGGCCACG | 647 |
| 6853 | AGTCCCCGCCCCGGCCACGT | 648 |
| 6854 | GTCCCCGCCCCGGCCACGTG | 649 |
| 6855 | TCCCCGCCCCGGCCACGTGG | 650 |
| 6856 | CCCCGCCCCGGCCACGTGGG | 651 |
| 6857 | CCCGCCCCGGCCACGTGGGC | 652 |
| 6858 | CCGCCCCGGCCACGTGGGCC | 653 |
| 6859 | CGCCCCGGCCACGTGGGCCT | 654 |
| 6860 | GCCCCGGCCACGTGGGCCTC | 655 |
| 6861 | CCCCGGCCACGTGGGCCTCC | 656 |
| 6862 | CCCGGCCACGTGGGCCTCCG | 657 |
| 6863 | CCGGCCACGTGGGCCTCCGA | 658 |
| 6864 | CGGCCACGTGGGCCTCCGAA | 659 |
| 6865 | GGCCACGTGGGCCTCCGAAC | 660 |
| 6866 | GCCACGTGGGCCTCCGAACC | 661 |
| 6867 | CCACGTGGGCCTCCGAACCA | 662 |
| 6868 | CACGTGGGCCTCCGAACCAC | 663 |
| 6869 | ACGTGGGCCTCCGAACCACG | 664 |
| 6870 | CGTGGGCCTCCGAACCACGA | 665 |
| 6871 | GTGGGCCTCCGAACCACGAG | 666 |
| 6872 | TGGGCCTCCGAACCACGAGT | 667 |
| 6873 | GGGCCTCCGAACCACGAGTC | 668 |
| 6874 | GGCCTCCGAACCACGAGTCA | 669 |
| 6875 | GCCTCCGAACCACGAGTCAT | 670 |
| 6876 | CCTCCGAACCACGAGTCATG | 671 |
| 6877 | CTCCGAACCACGAGTCATGC | 672 |
| 6878 | TCCGAACCACGAGTCATGCG | 673 |
| 6879 | CCGAACCACGAGTCATGCGG | 674 |
| 6880 | CGAACCACGAGTCATGCGGC | 675 |
| 6881 | GAACCACGAGTCATGCGGCA | 676 |
| 6882 | AACCACGAGTCATGCGGCAG | 677 |
| 6883 | ACCACGAGTCATGCGGCAGG | 678 |
| 6884 | CCACGAGTCATGCGGCAGGC | 679 |
| 6885 | CACGAGTCATGCGGCAGGCC | 680 |
| 6886 | ACGAGTCATGCGGCAGGCCG | 681 |
| 6887 | CGAGTCATGCGGCAGGCCGC | 682 |
| 6888 | GAGTCATGCGGCAGGCCGCA | 683 |
| 6889 | AGTCATGCGGCAGGCCGCAC | 684 |
| 6890 | GTCATGCGGCAGGCCGCACC | 685 |
| 6891 | TCATGCGGCAGGCCGCACCC | 686 |
| 6892 | CATGCGGCAGGCCGCACCCA | 687 |
| 6893 | ATGCGGCAGGCCGCACCCAG | 688 |
| 6894 | TGCGGCAGGCCGCACCCAGA | 689 |
| 6895 | GCGGCAGGCCGCACCCAGAC | 690 |
| 6896 | CGGCAGGCCGCACCCAGACC | 691 |
| 6897 | GGCAGGCCGCACCCAGACCC | 692 |
| 6898 | GCAGGCCGCACCCAGACCCG | 693 |
| 6899 | CAGGCCGCACCCAGACCCGC | 694 |
| 6900 | AGGCCGCACCCAGACCCGCC | 695 |
| 6901 | GGCCGCACCCAGACCCGCCC | 696 |
| 6902 | GCCGCACCCAGACCCGCCCC | 697 |
| 6903 | CCGCACCCAGACCCGCCCCT | 698 |
| 6904 | CGCACCCAGACCCGCCCCTC | 699 |
| 6905 | GCACCCAGACCCGCCCCTCC | 700 |
| 6906 | CACCCAGACCCGCCCCTCCC | 701 |
| 6907 | ACCCAGACCCGCCCCTCCCA | 702 |
| 6908 | CCCAGACCCGCCCCTCCCAC | 703 |
| 6909 | CCAGACCCGCCCCTCCCACA | 704 |
| 6910 | CAGACCCGCCCCTCCCACAC | 705 |
| 6911 | AGACCCGCCCCTCCCACACG | 706 |
| 6912 | GACCCGCCCCTCCCACACGG | 707 |
| 6913 | ACCCGCCCCTCCCACACGGG | 708 |
| 6914 | CCCGCCCCTCCCACACGGGA | 709 |
| 6915 | CCGCCCCTCCCACACGGGAC | 710 |
| 6916 | CGCCCCTCCCACACGGGACG | 711 |
| 6917 | GCCCCTCCCACACGGGACGT | 712 |
| 6918 | CCCCTCCCACACGGGACGTT | 713 |
| 6919 | CCCTCCCACACGGGACGTTT | 714 |
| 6920 | CCTCCCACACGGGACGTTTC | 715 |
| 6921 | CTCCCACACGGGACGTTTCA | 716 |
| 6922 | TCCCACACGGGACGTTTCAA | 717 |
| 6923 | CCCACACGGGACGTTTCAAT | 718 |
| 6924 | CCACACGGGACGTTTCAATA | 719 |
| 6925 | CACACGGGACGTTTCAATAA | 720 |
| 6926 | GCCCGCTACGTAATCAGTCG | 612 |
| 6927 | CGCCCGCTACGTAATCAGTC | 611 |
| 6928 | CCGCCCGCTACGTAATCAGT | 610 |
| 6929 | CCCGCCCGCTACGTAATCAG | 609 |
| 6930 | CCCCGCCCGCTACGTAATCA | 608 |
| 6931 | GCCCCGCCCGCTACGTAATC | 607 |
| 6932 | GGCCCCGCCCGCTACGTAAT | 606 |
| 6933 | CGGCCCCGCCCGCTACGTAA | 605 |
| 6934 | CCGGCCCCGCCCGCTACGTA | 604 |
| 6935 | TCCGGCCCCGCCCGCTACGT | 603 |
| 6936 | TTCCGGCCCCGCCCGCTACG | 602 |
| 6937 | CTTCCGGCCCCGCCCGCTAC | 601 |
| 6938 | ACTTCCGGCCCCGCCCGCTA | 600 |
| 6939 | CACTTCCGGCCCCGCCCGCT | 599 |
| 6940 | GCACTTCCGGCCCCGCCCGC | 598 |
| 6941 | GGCACTTCCGGCCCCGCCCG | 597 |
| 6942 | CGGCACTTCCGGCCCCGCCC | 596 |
| 6943 | GCGGCACTTCCGGCCCCGCC | 595 |
| 6944 | AGCGGCACTTCCGGCCCCGC | 594 |
| 6945 | GAGCGGCACTTCCGGCCCCG | 593 |
| 6946 | GGAGCGGCACTTCCGGCCCC | 592 |
| 6947 | AGGAGCGGCACTTCCGGCCC | 591 |
| 6948 | AAGGAGCGGCACTTCCGGCC | 590 |
| 6949 | CAAGGAGCGGCACTTCCGGC | 589 |
| 6950 | CCAAGGAGCGGCACTTCCGG | 588 |
| 6951 | ACCAAGGAGCGGCACTTCCG | 587 |
| 6952 | CACCAAGGAGCGGCACTTCC | 586 |
| 6953 | CCACCAAGGAGCGGCACTTC | 585 |
| 6954 | CCCACCAAGGAGCGGCACTT | 584 |
| 6955 | CCCCACCAAGGAGCGGCACT | 583 |
| 6956 | CCCCCACCAAGGAGCGGCAC | 582 |
| 6957 | GCCCCCACCAAGGAGCGGCA | 581 |
| 6958 | AGCCCCCACCAAGGAGCGGC | 580 |
| 6959 | CAGCCCCCACCAAGGAGCGG | 579 |
| 6960 | ACAGCCCCCACCAAGGAGCG | 578 |
| 6961 | AACGCAAAAACACCGGATTAATATCGGCCT | 142 |
| 6962 | GAACGCAAAAACACCGGATT | 141 |
| 6963 | ATAAACGGCCTCTTTACCCAGAGATCA | 850 |
| 6964 | TAAACGGCCTCTTTACCCAG | 851 |
| 6965 | AAACGGCCTCTTTACCCAGA | 852 |
| 6966 | AACGGCCTCTTTACCCAGAG | 853 |
| 6967 | ACGGCCTCTTTACCCAGAGA | 854 |
| 6968 | CGGCCTCTTTACCCAGAGAT | 855 |
| 6969 | GATAAACGGCCTCTTTACCC | 849 |
| 6970 | AGATAAACGGCCTCTTTACC | 848 |
| 6971 | CGCCACCTTAAGTTTTTCCAGGCTGC | 1779 |
| 6972 | GCGCCACCTTAAGTTTTTCC | 1778 |
| 6973 | GGCGCCACCTTAAGTTTTTC | 1777 |
| 6974 | AGGCGCCACCTTAAGTTTTT | 1776 |
| 6975 | AAGGCGCCACCTTAAGTTTT | 1775 |
| 6976 | TAAGGCGCCACCTTAAGTTT | 1774 |
| 6977 | ATAAGGCGCCACCTTAAGTT | 1773 |
| 6978 | TATAAGGCGCCACCTTAAGT | 1772 |
| 6979 | CTATAAGGCGCCACCTTAAG | 1771 |
| 6980 | ACTATAAGGCGCCACCTTAA | 1770 |
| 6981 | TACTATAAGGCGCCACCTTA | 1769 |
| 6982 | ATACTATAAGGCGCCACCTT | 1768 |
| 6983 | GATACTATAAGGCGCCACCT | 1767 |
| 6984 | TGATACTATAAGGCGCCACC | 1766 |
| 6985 | TTGATACTATAAGGCGCCAC | 1765 |

Hot Zones (Relative upstream location to gene start site)
1-950
1700-2000

### Examples

Genetic Code (5′ Upstream Region)

(SEQ ID NO: 11974)

CCACATCCACAAAGCACACCATTAATCCACTATGATCAAGTTGGGGGGAA
TCTGGTGAAGGGTTCTGAATATCTCCCTCTTCATCCCTCCCGAAATCTGG
AATACTTATTCTATTGAGCTATTACACCAGTTTTAACACCTTCCTCGTGT
TATGTTTAAAAAAATAAATAAATTTAAGAAAACCATTTTAAATAATGCAC
AGTTGCAGCCTGGAAAAACTTAAGGTGGCGCCTTATAGTATCAATTTTAG
GAGCTTTATTTGGTGCATTTAACGCAACTGGTAATTGCAGAATCCACTTT
GCCTGTGTAAGTGAAAAATATAGACTGTTATCTTGTTGGCCCTATGAAAT
TCTGCACTTTTCATTATATACTCTACCTTCATTAATTACTTCTGGCAAGA
TGTTCTGCCTTAGCACTCAGTTGCATTCTTTTCCTTTTTCTTCCTGTTCA
TTATGCTTTAATTCTGAGGACCATATGAGGGTAGAATATATTATCTTTTA
AAAATTACAAAAATTTGTATAGGCAAACCATTTCTTAAAGTTGATGGCCA
AATTTTAAAATGTTATTTTTCATATCATTTATAATCTTGTCACAATCCAC
TTAAAGAAGTTTGGTTATATTTCAGTGAAAATTTTCTTCCAGAGTAGGTT
TTTTTTCGTGGGTTGGGGGGTAACTTTACTACAATTAGTAAGTATGGTGC
AGAATTTCATGCAAATGAGGAGTGCCAGCAGTGTGATAATTTAAACATAT
TTAAACAAAAACAAAAAAAATGAATGCACAAACTTGCTGCTGCTTAGATC
ACTGCAGCTTCTAGGACCCGGTTTCTTTTACTGATTTAAAAACAAAACAA
AAAAAAATAAAAAAGTTGTGCCTGAAATGAATCTTGTTTTTTTTTATAAG
TAGCCGCCTGGTTACTGTGTCCTGTAAAATACAGACACTTGACCCTTGGT
GTAGCTTCTGTTCAACTTTATATCACGGGAATGGATGGGTCTGATTTCTT
GGCCCTCTTCTTGAATTGGCCATATACAGGGTCCCTGGCCAGTGGACTGA
AGGCTTTGTCTAAGATGACAAGGGTCAGCTCAGGGGATGTGGGGGAGGGC
GGTTTTATCTTCCCCCTTGTCGTTTGAGGTTTTGATCTCTGGGTAAAGAG
GCCGTTTATCTTTGTAAACACGAAACATTTTTGCTTTCTCCAGTTTTCTG
TTAATGGCGAAAGAATGGAAGCGAATAAAGTTTTACTGATTTTTGAGACA
CTAGCACCTAGCGCTTTCATTATTGAAACGTCCCGTGTGGGAGGGGCGGG
TCTGGGTGCGGCCTGCCGCATGACTCGTGGTTCGGAGGCCCACGTGGCCG
GGGCGGGGACTCAGGCGCCTGGGGCGCCGACTGATTACGTAGCGGGCGGG
GCCGGAAGTGCCGCTCCTTGGTGGGGGCTGTTCATGGCGGTTCCGGGGTC
TCCAACATTTTTCCCGGCTGTGGTCCTAAATCTGTCCAAAGCAGAGGCAG
TGGAGCTTGAGGTAAGTTTATCTCATGCATAGTGTTCGGCTTTGGGCTGT
GGAATGTTCAGGCGTTTCACTGATGCCAGAAATGGAGCAGAATCTATCAG
CTGGAGACAAAGGCCTTGGGCGGGGGTCCTTCCATTTGGTGCCTACGTGG
GGAGATCTTTGGAGACAGAAGGGAGAATGGGAAGGAGTTGCGGCCTGGAG
GCTTCCTGCTAGAGCTGAGAAGCCTTCGGGGAGTAATAGGAAGGGGGATT
TCCATTGCTTAGGCTGAGGGCGGGGCCCAAGGACTGTTGAAAAATAGCTA
AGGATGGGGGTTGCTAGAAAACTACTCCAGAAGTGTGAGGCCGATATTAA
TCCGGTGTTTTTGCGTTCTCTAGTCACTTTAAGAACCAAATGGAAGGTCA
CACTAGGGTTTTCATTTCCATTGATTATAGAAAGCTTTAAAGTACTGTAG
ATGTGGCTCGCCAATTAACCCTGATTACTGGTTTCCAACAGGTTCTTGCT
GGTGTGAAATG

26. EZH2. Histone-lysine N-methyltransferase (EZH2) is an enzyme that belongs to the Polycomb-group (PcG) family. PcG family members form multimeric protein complexes, which are involved in maintaining the transcriptional repressive state of genes over successive cell generations. EZH2 acts mainly as a gene silencer; it performs this role by the addition of three methyl groups to Lysine 27 of histone 3, a modification leading to chromatin condensation (Cao et al., 2002, Science 298 (5595): 1039-43). Mutations in in the EZH2 gene cause Weaver syndrome (Gibson et al., 2011: Am J Hum Genet 90 (1): 110-8). EZH2 overproduction may cause cancer due to increase in histone methylation. This histone methylation may play a role in silencing the expression of tumor suppressor genes, which may cause certain cancers. The microRNA produced by miR-101 normally inhibits translation of the messenger RNA coding for EZH2. Loss of this microRNA gene therefore leads to increased production of EZH2.

Protein: EZH2 Gene: EZH2 (Homo sapiens, chromosome 7, 148504464-148581441 [NCBI Reference Sequence: NC_{—}000007.13]; start site location: 148544390; strand: negative)

Gene Identification

| | |
|---|---|
| GeneID | 2146 |
| HGNC | 3527 |
| HPRD | 03342 |
| MIM | 601573 |

Targeted Sequences

| | | Relative |
|---|---|---|
| | | upstream |
| | | location to |
| Sequence | | gene |
| ID No: | Sequence (5′-3′) | start site |
| 6986 | TCCCGACAAGGGGTGACAGAGGC | 1002 |
| 7002 | CGTGAATTCAAGAGTTGCTTAGGCC | 1059 |
| 7003 | GACTACCGGTGCCCGCCACCACGCCAGGC | 2856 |
| 7035 | CCCCCGCCAACCCCACAGCGGATGCCCCC | 34593459 |
| | CGCCAACCCCACAGCGGATGC | |

Target Shift Sequences

| | | Relative |
|---|---|---|
| | | upstream |
| | | location to |
| Sequence | | gene |
| No: | Sequence (5′-3′) | start site |
| 6986 | TCCCGACAAGGGGTGACAGAGGC | 1002 |
| 6987 | ATCCCGACAAGGGGTGACAG | 1001 |
| 6988 | CATCCCGACAAGGGGTGACA | 1000 |
| 6989 | GCATCCCGACAAGGGGTGAC | 999 |
| 6990 | AGCATCCCGACAAGGGGTGA | 998 |
| 6991 | CAGCATCCCGACAAGGGGTG | 997 |
| 6992 | ACAGCATCCCGACAAGGGGT | 996 |
| 6993 | CACAGCATCCCGACAAGGGG | 995 |
| 6994 | GCACAGCATCCCGACAAGGG | 994 |
| 6995 | AGCACAGCATCCCGACAAGG | 993 |
| 6996 | CAGCACAGCATCCCGACAAG | 992 |
| 6997 | GCAGCACAGCATCCCGACAA | 991 |
| 6998 | TGCAGCACAGCATCCCGACA | 990 |
| 6999 | CTGCAGCACAGCATCCCGAC | 989 |
| 7000 | GCTGCAGCACAGCATCCCGA | 988 |
| 7001 | TGCTGCAGCACAGCATCCCG | 987 |
| 7002 | CGTGAATTCAAGAGTTGCTTAGGCC | 1059 |
| 7003 | GACTACCGGTGCCCGCCACCACGCCAGGC | 2856 |
| 7004 | ACTACCGGTGCCCGCCACCA | 2857 |
| 7005 | CTACCGGTGCCCGCCACCAC | 2858 |
| 7006 | TACCGGTGCCCGCCACCACG | 2859 |
| 7007 | ACCGGTGCCCGCCACCACGC | 2860 |
| 7008 | CCGGTGCCCGCCACCACGCC | 2861 |
| 7009 | CGGTGCCCGCCACCACGCCA | 2862 |
| 7010 | GGTGCCCGCCACCACGCCAG | 2863 |
| 7011 | GTGCCCGCCACCACGCCAGG | 2864 |
| 7012 | TGCCCGCCACCACGCCAGGC | 2865 |
| 7013 | GCCCGCCACCACGCCAGGCT | 2866 |
| 7014 | CCCGCCACCACGCCAGGCTA | 2867 |
| 7015 | CCGCCACCACGCCAGGCTAA | 2868 |
| 7016 | CGCCACCACGCCAGGCTAAT | 2869 |
| 7017 | GCCACCACGCCAGGCTAATT | 2870 |
| 7018 | CCACCACGCCAGGCTAATTT | 2871 |
| 7019 | CACCACGCCAGGCTAATTTT | 2872 |
| 7020 | ACCACGCCAGGCTAATTTTT | 2873 |
| 7021 | CCACGCCAGGCTAATTTTTT | 2874 |
| 7022 | CACGCCAGGCTAATTTTTTG | 2875 |
| 7023 | GGACTACCGGTGCCCGCCAC | 2855 |
| 7024 | GGGACTACCGGTGCCCGCCA | 2854 |
| 7025 | TGGGACTACCGGTGCCCGCC | 2853 |
| 7026 | GTGGGACTACCGGTGCCCGC | 2852 |
| 7027 | GGTGGGACTACCGGTGCCCG | 2851 |
| 7028 | AGGTGGGACTACCGGTGCCC | 2850 |
| 7029 | TAGGTGGGACTACCGGTGCC | 2849 |
| 7030 | GTAGGTGGGACTACCGGTGC | 2848 |
| 7031 | AGTAGGTGGGACTACCGGTG | 2847 |
| 7032 | AAGTAGGTGGGACTACCGGT | 2846 |
| 7033 | CAAGTAGGTGGGACTACCGG | 2845 |
| 7034 | CCAAGTAGGTGGGACTACCG | 2844 |
| 7035 | GACCGCCCCCCGCCAACCCCACAGCGG | 3453 |
| 7036 | ACCGCCCCCCGCCAACCCCA | 3454 |
| 7037 | CCGCCCCCCGCCAACCCCAC | 3455 |
| 7038 | CGCCCCCCGCCAACCCCACA | 3456 |
| 7039 | GCCCCCCGCCAACCCCACAG | 3457 |
| 7040 | CCCCCCGCCAACCCCACAGC | 3458 |
| 7041 | CCCCCGCCAACCCCACAGCG | 3459 |
| 7042 | CCCCGCCAACCCCACAGCGG | 3460 |
| 7043 | CCCGCCAACCCCACAGCGGA | 3461 |
| 7044 | CCGCCAACCCCACAGCGGAT | 3462 |
| 7045 | CGCCAACCCCACAGCGGATG | 3463 |
| 7046 | GCCAACCCCACAGCGGATGC | 3464 |
| 7047 | CCAACCCCACAGCGGATGCC | 3465 |
| 7048 | CAACCCCACAGCGGATGCCT | 3466 |
| 7049 | AACCCCACAGCGGATGCCTA | 3467 |
| 7050 | ACCCCACAGCGGATGCCTAA | 3468 |
| 7051 | CCCCACAGCGGATGCCTAAA | 3469 |
| 7052 | CCCACAGCGGATGCCTAAAG | 3470 |
| 7053 | CCACAGCGGATGCCTAAAGC | 3471 |
| 7054 | CACAGCGGATGCCTAAAGCT | 3472 |
| 7055 | ACAGCGGATGCCTAAAGCTG | 3473 |
| 7056 | CAGCGGATGCCTAAAGCTGC | 3474 |
| 7057 | AGCGGATGCCTAAAGCTGCA | 3475 |
| 7058 | GCGGATGCCTAAAGCTGCAG | 3476 |
| 7059 | CGGATGCCTAAAGCTGCAGA | 3477 |
| 7060 | AGACCGCCCCCCGCCAACCC | 3452 |
| 7061 | AAGACCGCCCCCCGCCAACC | 3451 |
| 7062 | CAAGACCGCCCCCCGCCAAC | 3450 |
| 7063 | CCAAGACCGCCCCCCGCCAA | 3449 |
| 7064 | CCCAAGACCGCCCCCCGCCA | 3448 |
| 7065 | TCCCAAGACCGCCCCCCGCC | 3447 |
| 7066 | CTCCCAAGACCGCCCCCCGC | 3446 |
| 7067 | TCTCCCAAGACCGCCCCCCG | 3445 |
| 7068 | ATCTCCCAAGACCGCCCCCC | 3444 |
| 7069 | TATCTCCCAAGACCGCCCCC | 3443 |
| 7070 | TTATCTCCCAAGACCGCCCC | 3442 |
| 7071 | CTTATCTCCCAAGACCGCCC | 3441 |
| 7072 | ACTTATCTCCCAAGACCGCC | 3440 |
| 7073 | CACTTATCTCCCAAGACCGC | 3439 |
| 7074 | CCACTTATCTCCCAAGACCG | 3438 |

```
Hot Zones (Relative upstream location to gene start site)
  1-300
  900-1100
2600-3100
3400-4200
```

### Examples

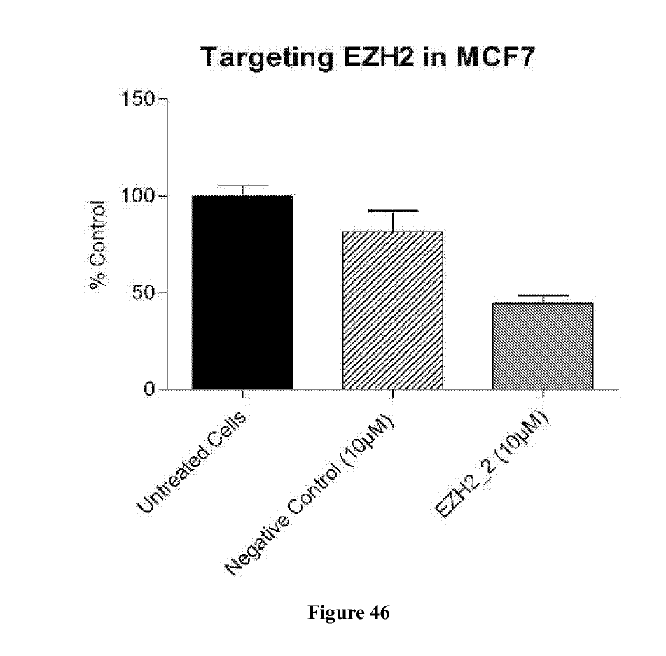

In FIG. 46, In MCF7 (human mammary breast cell line), EZH2_{—}2 (271) produced statistically significant (P<0.05) inhibition at 10 μM compared to the untreated and negative control values. The EZH2 sequence EZH2_{—}2 (271) fits the independent and dependent DNAi motif claims.

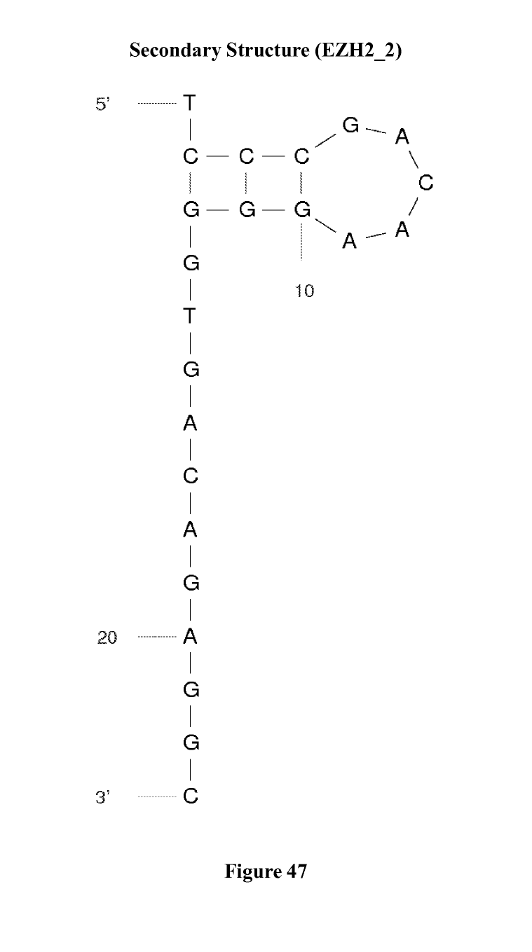

The secondary structure for EZH2_{—}2 (271) is shown in FIG. 47.

Genetic Code (5′ Upstream Region)

(SEQ ID NO: 11975)

CACAGGCTCAAGAGATCCTCCCACCTCAGCTTCCTGAGTAGTTGGGACCA
CAGGTGTGCGCCACTACACCTGGCTTGCTTGCTTGCTTATTTATTGATTT
GAGATGGGAGTCTCACTATATTGCCTAGGCTGGTCTTGAACTCCTGGGCT
CAATCCTCCAACCTTGGCCTCCCAAAATGCTGGGTTTACAGGCTTGAACC
ACTGTACGTGGCCTTGAATCTGTGTTTTAATACTATGCTTACTTGGCTGT
GGTGTTGTGAAAAGATCACTGAAAATGGAGTCAGAGGCCTGATTTGAGCC
AGTCGTTTGTTGTGGGGGAAGGAGGTCAGGGGAGCTAACATCTAAAGGCT
CACTATATGCCAGGCACAGAACCAAGTGTGTTTGCATGTATATTTCGTTT
TTGTTGCCAGACTTTGAGGTAGGTTTTATGGATAAGGTCTTTAAGGCAAT
ATCAGCTTCCTTTTAAAAAAGAAATTCCGGAAACTGAGTTTTAGGCTGAA
GATCTCTAACTGGTAGTAGGGACAACTGAACCACAGGGTCCTAACTGACC
CTGCGATTTATCTCCTTTTGCGGGGGGTTTCTTGATAATAGGGTGCACTT
TACCTCATTTTTTGGCTCAAGCATGGATAGGCCACCCTTCCTTTTCATAC
CTATAGCTAAGCTTTACAAATGATATGCTGATAAGATACAAGCTACTCGT
TATTCATGTGGGTTAATAGACCTGTTTGTTTGCTTGTTTTTAAGTCTATA
GCCGCCCCACCCCCAATCTACAATTTCACCTTCTAAGGTTTTAGTTACTC
ATTCAAACTGCAGTCTGAAAATGTTACGATATTTTGAGAGAGAGAAGACT
CTAGCTACGTAACTTTTGTAACAATATATTGTTATAATTGTTCATTTTAC
CATTAGTTATTGCTGTCAGTCTCTTACTGTGCCTTATTTATAAATTAAAC
TTCATGGGTATGTACGTATAGGAAAAAACATGGTATATTTAGAGTTTAGT
ACTATCTGCAGCTTTAGGCATCCGCTGTGGGGTTGGCGGGGGGCGGTCTT
GGGAGATAAGTGGGGACTACTGTACAATTATCAGGCACACACAGGCTCTG
GGATTTTACAAATGAGTAAAAGTGGTTCTTGCTGTTGAAGCACTTACAGT
GGGAATAGAGTGAAATACATGAAAATGTGATTTTAATATGTTATAAATGC
TATGATGGTGGGAGTTTGTTTTGTGTAAAACATCCTTTTAATTGGTACTT
TAAATTTTAATATTCTTTCACAGGTCTACCTATTTAGTCTTACACTTTCA
AAGAACTACCTGGATGCTGTAGATTTTCATGATATACTTTATTAGGTATG
TTATTAATGGTAGAAACAGCATGGAAAGTCTTCCAGAATATTAGACAAGG
ACAGTTCTAGTACTAAAACATAAAATGCTAACTAATGTCTTCATCAAGAC
ATAAAATATGTATCTTAAAAAATAAATTGTAAGCCAGGCGCAGTGGCTCA
CACCTGTAATCCCAGCACTTTGGGAGGCTGAGGCGGGTGGATCACAAGGT
CAGGAGATTGAGACCATCCTGGCTAACACGGTGAAACCCTGTCTCTACTA
AAAATACAAAAAATTAGCCTGGCGTGGTGGCGGGCACCGGTAGTCCCACC
TACTTGGGAGGCTGAGGCAGGAGAATGGCGTGAACCCGGGAGGCAGAGCT
TGCAGTGAGCGGAGATTGCACCACCGCACTCCAGCTTGGGGGACAGAGTG
AGACTCCATCTCAAAAAAAAAGAAATTGTAATAACACCCACATTATACAT
CAGTGAAAACTAAACACGTTACTACCCTAGGCCTTATTGCACAGGGGTGC
TACCTCCAAGGAGAAATTTGTCTAGGCAGCAGATGGACTAGAGGTGATTA
GCCTATGAGCGAATGAGGCTACAGATCATTCCTTTTTATCTGATTCCTTT
TCTTTCTAGTTCCTAGGCCTTGGAAGCACTAAGTGGTCTTAAGTAATTTG
CATAGAATTAGTTGAGTTCATCTGTTAACTAACTAGCAGATAGGAAGAAA
ACTATTGTCATGAAATTATTTAAAAAATAATAATGCTCCAGTTTCTTCTC
ATCTTTGATGTCCTTTGGTCCTACCTCACTGCCTTCCTAACACCATTTTC
TGCTTTACCTCAAAGCTGGGGTCATCTTGAGTTTAGCCTGCTTAATCCGA
GTGACTGTCAGCTTTATTCCACTTTAGCAACTCGCAGGCAAGGCCACACT
TGGAAACTTTTCACTTGGAATAGTTCTATCTTGGTGATTTCATCAGCCTT
CTTTATGTCAAATACACTCAAATTCCTGCCCATTCTTATCTCTTCGTTCC
CTTCAGGTCCTTAGTCCTTTTAATTTGTGACTTTCATTCTCCAGGTCCAT
TCTTATTAAATGTCTGCCAGCCAGACTTTAGTTGCCCCCTGTCCAGCTTT
CTCTTGCTCAGACCTAAGATTTCTTTAGGTTCTTTCTTTGCCTTTTGAAA
TCCAGCTCAGCTTTTAAGATTGAGTTCCTTGTTACCTTTCCTGCCATCCT
TCTGCAGTTCCTAATATTCTTTTCTTTCTCCCAAAGTGCTTTTGTATAAA
CAGTCAGCCTTCCATATCCGTGAGTTCCAAATCCATGGATCCTGAATTCA
TGGATTTAACCAGCTGCAGATAAAAAATATTCAGAAAAAAAAAGATGGTT
GCATCTGTACTGAACATGTCTGTACTGTTTTGCTTGTCATTATTTTCTAA
ACAATACAGTATAACAACTATTTACATGGCATTTACATTGTATTAGGGAT
TATAAGTAATCTAGAGGTGATTTAAAGTATATGGGAGTCTCTTATATCCC
AGGAAGCCAGGTAAAAAAAAAAAGTATATGGGAGGCTATGCATAGGTGAT
ATGCAAATATTACACCACTTTATATCACGGACTTTTGAGCATCTGTGGAT
TTTGGTATCCGAGGGGTGTCCTGGAACCAGTTCCCCAGGGATACTGAAGT
ATGTCTGTCTATCTCATACTATATTTTTCCCTTTGTCTTAGGTAGACTAT
CAGCTCCATAGGGGCAAGGATTTAATAATATTTGTATATTCATTTTATTC
AAGTTCGTATACACTGCTTGGGTCATAATATTTATTACATGCTTGAGAAA
ATGAATTTCTTCGCCCCTTTGTTACAGCTCTGAGTAAACAGCCATCTGCC
TTCTCTGTCATCTGTTGGTGGTTGAGTATTTCTGTAGAAAGTTACCCATT
GGCCTCAGGACTCTTACTCTAAATCTTCTTCTTAGGCAGTTTTCTCTGTG
CATGAAGTTTTTATGTAAACAAATAGATGAAGCCTGCCCTACTCATTTAT
TTGCTCAAGCCAGAAAGTCACCTTCTTCTTCACTTTCCATATTTAAATCA
TCATTTGGTGGAATTTTGGCCTAAGCAACTCTTGAATTCACGTACTTTTC
CCTGTCATCGCCAGTGTGGTGTAGAAGCCTCTGTCACCCCTTGTCGGGAT
GCTGTGCTGCAGCATCATCTAACCTGGTTGCAGTTATTCTTTCACTCCCT
CACCGCACACCCTTTTACTTAAAACACTAAAAGTGGCTTCTCATTGTTCT
TAAGATAAAGCACAAATTGTTAGTGTGGCCTGTAAAGCTTTGCATAGCCT
GACAGAGAATGTCCTGCTAATAATTTGAAGGTACAGGATGATTTTAATAC
TTTAGGAGAAAATGTTCTAGGAAAAGACGCTTGTTTAGACTTAAGGTGAG
GACTCTGCAGTATGAATTAGACATCTGGTGAACTATAAGCTGTCCCCGCA
TTTAAACATAATTGGTTCTGAGAGCCTGCAACTAAAGATAAGGCAGAAGA
ATTTACTTTGCATTTCCTGCATTCCTCTTTTCGCTTGATAGCAGAAACCC
CTCATGTTAATAAAGGTGGCACAAGAGGCAAAAATACAGACTTTATCACA
GTGTTTAAGGAGAGGTGCATGATTAAGTGTGTGGGGAGAGAGTACCTTTG
TACATTTTATTATATGGTGAACTGTATGTTTTCTACTTTTAGTACTGTTT
GTAAATTTTACTTCTTCTTGGATTTACCTTTTTCAGTTATATTATTCCAT
TATGCCTTGCTACTGTAACAGCTAATGATGAAAAACAGGATCTGTCTTTA
TATTTTCTTCCCTCCACAAATGTGGATCTCATAGAGTTGAAAACTAGGTT
GTGATATAGTATAGTATACCTAATTCCTGTAATGGGATCATGTTCCTATA
ATATGGCCGCAATTTAGTGTAGAATTTTTGTAAATAAAAGTGTATTTTAA
GTTTAACTTAAACTTTCAATGAAGTGTTTTAAGGATTTAACCATGCAGCA
CAAATGAGCACCTTTCTGTAAATGCCAACAGTGTAATATGTGTCATTTCT
TCACTGATTGTTAGTTTGCTGCGGATTAAAACACAGGTGATCATATTCAG
GCTGGTTAGATTAGTGATTTTAATATGAAACCATTGCTTTTAGAATAATC
ATG

27. HDACs, such as HDAC1.

Histone deacetylases (HDACs) are part of a vast family of enzymes that have crucial roles in numerous biological processes, largely through their repressive influence on transcription (reviewed by Haberland et al, 2009 Nature Reviews Genetics 10, 32-42. HDAC1 is an enzyme that belongs the histone deacetylase family and is a component of the histone deacetylase complex (Taunton et al, Science 272 (5260): 408-11) Histone acetylation and deacetylation, is catalyzed by multisubunit complexes and is key in the expression of gene expression. It also interacts with retinoblastoma tumor-suppressor protein and this complex is a key element in the control of cell proliferation and differentiation. Together with metastasis-associated protein-2 MTA2, it deacetylates p53 and modulates its effect on cell growth and apoptosis.

Protein: HDAC1 Gene: HDAC1 (Homo sapiens, chromosome 1, 32757708-32799224 [NCBI Reference Sequence: NC_{—}000001.10]; start site location: 32757771; strand: positive)

Gene Identification

| | |
|---|---|
| GeneID | 3065 |
| HGNC | 4852 |
| HPRD | 03143 |
| MIM | 601241 |

Targeted Sequences

| | | Relative |
|---|---|---|
| | | upstream |
| | | location to |
| Sequence | | gene |
| ID No: | Sequence (5′-3′) | start site |
| 7075 | CGCCTCCCGTCCCTACCGTCAGTCGGT | 7 |
| 7141 | CGGTCCGTCCGCCCTCCCGCCCGCGG | 30 |
| 7207 | CGCCAACTTGTGGTCCTACAGTCAACAAG | 1740 |
| 7226 | CGCAGACACGGGCCCGGAACTCGG | 173 |
| 7258 | CGCCCGGCCTAGGAGGGCAGGTTTCTC | 1252 |

Target Shift Sequences

| | | Relative |
|---|---|---|
| | | upstream |
| | | location to |
| Sequence | | gene |
| ID No: | Sequence (5′-3′) | start site |
| 7075 | CGCCTCCCGTCCCTACCGTCAGTCGGT | 7 |
| 7076 | GCCTCCCGTCCCTACCGTCA | 8 |
| 7077 | CCTCCCGTCCCTACCGTCAG | 9 |
| 7078 | CTCCCGTCCCTACCGTCAGT | 10 |
| 7079 | TCCCGTCCCTACCGTCAGTC | 11 |
| 7080 | CCCGTCCCTACCGTCAGTCG | 12 |
| 7081 | CCGTCCCTACCGTCAGTCGG | 13 |
| 7082 | CGTCCCTACCGTCAGTCGGT | 14 |
| 7083 | GTCCCTACCGTCAGTCGGTC | 15 |
| 7084 | TCCCTACCGTCAGTCGGTCC | 16 |
| 7085 | CCCTACCGTCAGTCGGTCCG | 17 |
| 7086 | CCTACCGTCAGTCGGTCCGT | 18 |
| 7087 | CTACCGTCAGTCGGTCCGTC | 19 |
| 7088 | TACCGTCAGTCGGTCCGTCC | 20 |
| 7089 | ACCGTCAGTCGGTCCGTCCG | 21 |
| 7090 | CCGTCAGTCGGTCCGTCCGC | 22 |
| 7091 | CGTCAGTCGGTCCGTCCGCC | 23 |
| 7092 | GTCAGTCGGTCCGTCCGCCC | 24 |
| 7093 | TCAGTCGGTCCGTCCGCCCT | 25 |
| 7094 | CAGTCGGTCCGTCCGCCCTC | 26 |
| 7095 | AGTCGGTCCGTCCGCCCTCC | 27 |
| 7096 | GTCGGTCCGTCCGCCCTCCC | 28 |
| 7097 | TCGGTCCGTCCGCCCTCCCG | 29 |
| 7098 | CGGTCCGTCCGCCCTCCCGC | 30 |
| 7099 | GGTCCGTCCGCCCTCCCGCC | 31 |
| 7100 | GTCCGTCCGCCCTCCCGCCC | 32 |
| 7101 | TCCGTCCGCCCTCCCGCCCG | 33 |
| 7102 | CCGTCCGCCCTCCCGCCCGC | 34 |
| 7103 | CGTCCGCCCTCCCGCCCGCG | 35 |
| 7104 | GTCCGCCCTCCCGCCCGCGG | 36 |
| 7105 | TCCGCCCTCCCGCCCGCGGC | 37 |
| 7106 | CCGCCCTCCCGCCCGCGGCT | 38 |
| 7107 | CGCCCTCCCGCCCGCGGCTC | 39 |
| 7108 | GCCCTCCCGCCCGCGGCTCC | 40 |
| 7109 | CCCTCCCGCCCGCGGCTCCG | 41 |
| 7110 | CCTCCCGCCCGCGGCTCCGC | 42 |
| 7111 | CTCCCGCCCGCGGCTCCGCT | 43 |
| 7112 | TCCCGCCCGCGGCTCCGCTC | 44 |
| 7113 | CCCGCCCGCGGCTCCGCTCA | 45 |
| 7114 | CCGCCCGCGGCTCCGCTCAG | 46 |
| 7115 | CGCCCGCGGCTCCGCTCAGC | 47 |
| 7116 | GCCCGCGGCTCCGCTCAGCG | 48 |
| 7117 | CCCGCGGCTCCGCTCAGCGT | 49 |
| 7118 | CCGCGGCTCCGCTCAGCGTC | 50 |
| 7119 | CGCGGCTCCGCTCAGCGTCC | 51 |
| 7120 | GCGGCTCCGCTCAGCGTCCG | 52 |
| 7121 | CGGCTCCGCTCAGCGTCCGA | 53 |
| 7122 | GGCTCCGCTCAGCGTCCGAC | 54 |
| 7123 | GCTCCGCTCAGCGTCCGACC | 55 |
| 7124 | CTCCGCTCAGCGTCCGACCC | 56 |
| 7125 | TCCGCTCAGCGTCCGACCCA | 57 |
| 7126 | CCGCTCAGCGTCCGACCCAG | 58 |
| 7127 | CGCTCAGCGTCCGACCCAGG | 59 |
| 7128 | GCTCAGCGTCCGACCCAGGG | 60 |
| 7129 | CTCAGCGTCCGACCCAGGGG | 61 |
| 7130 | TCAGCGTCCGACCCAGGGGG | 62 |
| 7131 | CAGCGTCCGACCCAGGGGGG | 63 |
| 7132 | AGCGTCCGACCCAGGGGGGA | 64 |
| 7133 | GCGTCCGACCCAGGGGGGAG | 65 |
| 7134 | CGTCCGACCCAGGGGGGAGG | 66 |
| 7135 | TCGCCTCCCGTCCCTACCGT | 6 |
| 7136 | CTCGCCTCCCGTCCCTACCG | 5 |
| 7137 | GCTCGCCTCCCGTCCCTACC | 4 |
| 7138 | TGCTCGCCTCCCGTCCCTAC | 3 |
| 7139 | TTGCTCGCCTCCCGTCCCTA | 2 |
| 7140 | CTTGCTCGCCTCCCGTCCCT | 1 |
| 7141 | CGGTCCGTCCGCCCTCCCGCCCGCGG | 30 |
| 7142 | GGTCCGTCCGCCCTCCCGCC | 31 |
| 7143 | GTCCGTCCGCCCTCCCGCCC | 32 |
| 7144 | TCCGTCCGCCCTCCCGCCCG | 33 |
| 7145 | CCGTCCGCCCTCCCGCCCGC | 34 |
| 7146 | CGTCCGCCCTCCCGCCCGCG | 35 |
| 7147 | GTCCGCCCTCCCGCCCGCGG | 36 |
| 7148 | TCCGCCCTCCCGCCCGCGGC | 37 |
| 7149 | CCGCCCTCCCGCCCGCGGCT | 38 |
| 7150 | CGCCCTCCCGCCCGCGGCTC | 39 |
| 7151 | GCCCTCCCGCCCGCGGCTCC | 40 |
| 7152 | CCCTCCCGCCCGCGGCTCCG | 41 |
| 7153 | CCTCCCGCCCGCGGCTCCGC | 42 |
| 7154 | CTCCCGCCCGCGGCTCCGCT | 43 |
| 7155 | TCCCGCCCGCGGCTCCGCTC | 44 |
| 7156 | CCCGCCCGCGGCTCCGCTCA | 45 |
| 7157 | CCGCCCGCGGCTCCGCTCAG | 46 |
| 7158 | CGCCCGCGGCTCCGCTCAGC | 47 |
| 7159 | GCCCGCGGCTCCGCTCAGCG | 48 |
| 7160 | CCCGCGGCTCCGCTCAGCGT | 49 |
| 7161 | CCGCGGCTCCGCTCAGCGTC | 50 |
| 7162 | CGCGGCTCCGCTCAGCGTCC | 51 |
| 7163 | GCGGCTCCGCTCAGCGTCCG | 52 |
| 7164 | CGGCTCCGCTCAGCGTCCGA | 53 |
| 7165 | GGCTCCGCTCAGCGTCCGAC | 54 |
| 7166 | GCTCCGCTCAGCGTCCGACC | 55 |
| 7167 | CTCCGCTCAGCGTCCGACCC | 56 |
| 7168 | TCCGCTCAGCGTCCGACCCA | 57 |
| 7169 | CCGCTCAGCGTCCGACCCAG | 58 |
| 7170 | CGCTCAGCGTCCGACCCAGG | 59 |
| 7171 | GCTCAGCGTCCGACCCAGGG | 60 |
| 7172 | CTCAGCGTCCGACCCAGGGG | 61 |
| 7173 | TCAGCGTCCGACCCAGGGGG | 62 |
| 7174 | CAGCGTCCGACCCAGGGGGG | 63 |
| 7175 | AGCGTCCGACCCAGGGGGGA | 64 |
| 7176 | GCGTCCGACCCAGGGGGGAG | 65 |
| 7177 | CGTCCGACCCAGGGGGGAGG | 66 |
| 7178 | TCGGTCCGTCCGCCCTCCCG | 29 |
| 7179 | GTCGGTCCGTCCGCCCTCCC | 28 |
| 7180 | AGTCGGTCCGTCCGCCCTCC | 27 |
| 7181 | CAGTCGGTCCGTCCGCCCTC | 26 |
| 7182 | TCAGTCGGTCCGTCCGCCCT | 25 |
| 7183 | GTCAGTCGGTCCGTCCGCCC | 24 |
| 7184 | CGTCAGTCGGTCCGTCCGCC | 23 |
| 7185 | CCGTCAGTCGGTCCGTCCGC | 22 |
| 7186 | ACCGTCAGTCGGTCCGTCCG | 21 |
| 7187 | TACCGTCAGTCGGTCCGTCC | 20 |
| 7188 | CTACCGTCAGTCGGTCCGTC | 19 |
| 7189 | CCTACCGTCAGTCGGTCCGT | 18 |
| 7190 | CCCTACCGTCAGTCGGTCCG | 17 |
| 7191 | TCCCTACCGTCAGTCGGTCC | 16 |
| 7192 | GTCCCTACCGTCAGTCGGTC | 15 |
| 7193 | CGTCCCTACCGTCAGTCGGT | 14 |
| 7194 | CCGTCCCTACCGTCAGTCGG | 13 |
| 7195 | CCCGTCCCTACCGTCAGTCG | 12 |
| 7196 | TCCCGTCCCTACCGTCAGTC | 11 |
| 7197 | CTCCCGTCCCTACCGTCAGT | 10 |
| 7198 | CCTCCCGTCCCTACCGTCAG | 9 |
| 7199 | GCCTCCCGTCCCTACCGTCA | 8 |
| 7200 | CGCCTCCCGTCCCTACCGTC | 7 |
| 7201 | TCGCCTCCCGTCCCTACCGT | 6 |
| 7202 | CTCGCCTCCCGTCCCTACCG | 5 |
| 7203 | GCTCGCCTCCCGTCCCTACC | 4 |
| 7204 | TGCTCGCCTCCCGTCCCTAC | 3 |
| 7205 | TTGCTCGCCTCCCGTCCCTA | 2 |
| 7206 | CTTGCTCGCCTCCCGTCCCT | 1 |
| 7207 | CGCCAACTTGTGGTCCTACAGTCAACAAG | 1740 |
| 7208 | CCGCCAACTTGTGGTCCTAC | 1739 |
| 7209 | GCCGCCAACTTGTGGTCCTA | 1738 |
| 7210 | AGCCGCCAACTTGTGGTCCT | 1737 |
| 7211 | TAGCCGCCAACTTGTGGTCC | 1736 |
| 7212 | TTAGCCGCCAACTTGTGGTC | 1735 |
| 7213 | GTTAGCCGCCAACTTGTGGT | 1734 |
| 7214 | AGTTAGCCGCCAACTTGTGG | 1733 |
| 7215 | AAGTTAGCCGCCAACTTGTG | 1732 |
| 7216 | TAAGTTAGCCGCCAACTTGT | 1731 |
| 7217 | CTAAGTTAGCCGCCAACTTG | 1730 |
| 7218 | TCTAAGTTAGCCGCCAACTT | 1729 |
| 7219 | CTCTAAGTTAGCCGCCAACT | 1728 |
| 7220 | GCTCTAAGTTAGCCGCCAAC | 1727 |
| 7221 | TGCTCTAAGTTAGCCGCCAA | 1726 |
| 7222 | TTGCTCTAAGTTAGCCGCCA | 1725 |
| 7223 | ATTGCTCTAAGTTAGCCGCC | 1724 |
| 7224 | CATTGCTCTAAGTTAGCCGC | 1723 |
| 7225 | ACATTGCTCTAAGTTAGCCG | 1722 |
| 7226 | CGCAGACACGGGCCCGGAACTCGG | 173 |
| 7227 | GCAGACACGGGCCCGGAACT | 174 |
| 7228 | CAGACACGGGCCCGGAACTC | 175 |
| 7229 | AGACACGGGCCCGGAACTCG | 176 |
| 7230 | GACACGGGCCCGGAACTCGG | 177 |
| 7231 | ACACGGGCCCGGAACTCGGC | 178 |
| 7232 | CACGGGCCCGGAACTCGGCA | 179 |
| 7233 | ACGGGCCCGGAACTCGGCAG | 180 |
| 7234 | CGGGCCCGGAACTCGGCAGG | 181 |
| 7235 | GGGCCCGGAACTCGGCAGGG | 182 |
| 7236 | GGCCCGGAACTCGGCAGGGG | 183 |
| 7237 | GCCCGGAACTCGGCAGGGGG | 184 |
| 7238 | CCCGGAACTCGGCAGGGGGC | 185 |
| 7239 | CCGGAACTCGGCAGGGGGCA | 186 |
| 7240 | GCGCAGACACGGGCCCGGAA | 172 |
| 7241 | TGCGCAGACACGGGCCCGGA | 171 |
| 7242 | TTGCGCAGACACGGGCCCGG | 170 |
| 7243 | CTTGCGCAGACACGGGCCCG | 169 |
| 7244 | GCTTGCGCAGACACGGGCCC | 168 |
| 7245 | AGCTTGCGCAGACACGGGCC | 167 |
| 7246 | CAGCTTGCGCAGACACGGGC | 166 |
| 7247 | TCAGCTTGCGCAGACACGGG | 165 |
| 7248 | ATCAGCTTGCGCAGACACGG | 164 |
| 7249 | AATCAGCTTGCGCAGACACG | 163 |
| 7250 | CAATCAGCTTGCGCAGACAC | 162 |
| 7251 | CCAATCAGCTTGCGCAGACA | 161 |
| 7252 | GCCAATCAGCTTGCGCAGAC | 160 |
| 7253 | AGCCAATCAGCTTGCGCAGA | 159 |
| 7254 | CAGCCAATCAGCTTGCGCAG | 158 |
| 7255 | CCAGCCAATCAGCTTGCGCA | 157 |
| 7256 | TCCAGCCAATCAGCTTGCGC | 156 |
| 7257 | CTCCAGCCAATCAGCTTGCG | 155 |
| 7258 | CGCCCGGCCTAGGAGGGCAGGTTTCTC | 1252 |
| 7259 | GCCCGGCCTAGGAGGGCAGG | 1253 |
| 7260 | CCCGGCCTAGGAGGGCAGGT | 1254 |
| 7261 | CCGGCCTAGGAGGGCAGGTT | 1255 |
| 7262 | CGGCCTAGGAGGGCAGGTTT | 1256 |
| 7263 | GCGCCCGGCCTAGGAGGGCA | 1251 |
| 7264 | AGCGCCCGGCCTAGGAGGGC | 1250 |
| 7265 | CAGCGCCCGGCCTAGGAGGG | 1249 |
| 7266 | ACAGCGCCCGGCCTAGGAGG | 1248 |
| 7267 | CACAGCGCCCGGCCTAGGAG | 1247 |
| 7268 | CCACAGCGCCCGGCCTAGGA | 1246 |
| 7269 | GCCACAGCGCCCGGCCTAGG | 1245 |
| 7270 | AGCCACAGCGCCCGGCCTAG | 1244 |
| 7271 | GAGCCACAGCGCCCGGCCTA | 1243 |
| 7272 | TGAGCCACAGCGCCCGGCCT | 1242 |
| 7273 | GTGAGCCACAGCGCCCGGCC | 1241 |
| 7274 | CGTGAGCCACAGCGCCCGGC | 1240 |
| 7275 | GCGTGAGCCACAGCGCCCGG | 1239 |
| 7276 | GGCGTGAGCCACAGCGCCCG | 1238 |
| 7277 | GGGCGTGAGCCACAGCGCCC | 1237 |
| 7278 | CGGGCGTGAGCCACAGCGCC | 1236 |
| 7279 | ACGGGCGTGAGCCACAGCGC | 1235 |
| 7280 | TACGGGCGTGAGCCACAGCG | 1234 |
| 7281 | TTACGGGCGTGAGCCACAGC | 1233 |
| 7282 | ATTACGGGCGTGAGCCACAG | 1232 |
| 7283 | GATTACGGGCGTGAGCCACA | 1231 |
| 7284 | AGATTACGGGCGTGAGCCAC | 1230 |
| 7285 | GAGATTACGGGCGTGAGCCA | 1229 |
| 7286 | TGAGATTACGGGCGTGAGCC | 1228 |
| 7287 | CTGAGATTACGGGCGTGAGC | 1227 |
| 7288 | GCTGAGATTACGGGCGTGAG | 1226 |
| 7289 | TGCTGAGATTACGGGCGTGA | 1225 |
| 7290 | ATGCTGAGATTACGGGCGTG | 1224 |
| 7291 | TATGCTGAGATTACGGGCGT | 1223 |
| 7292 | ATATGCTGAGATTACGGGCG | 1222 |
| 7293 | AATATGCTGAGATTACGGGC | 1221 |
| 7294 | CAATATGCTGAGATTACGGG | 1220 |
| 7295 | CCAATATGCTGAGATTACGG | 1219 |
| 7296 | CCCAATATGCTGAGATTACG | 1218 |

```
Hot Zones (Relative upstream location to gene start site)
  1-650
  850-1300
1700-2050
2250-2550
2800-3700
4350-5000
```

### Examples

Genetic Code (5′ Upstream Region)

(SEQ ID NO: 11976)

CCAGGCTGATCTCAAACTCCTAAGCTCAAGTGATCCATGTTCCTCAGCCT
CCCAAAGTGCTGGGATTATAGGCGTGAGCCATAGCGTCCAGCCCTGACTT
ACATTTTAAAAGGATGGCTCTTGCTGCTGTCTTGAAAATAGACTGAGTTA
GTCAGTTTATAAAACTGGGGAGATTTTGCATAAAACTCCAGATTTCTGCC
TTCTCTTGAAAAATAGGGGCTAGGTGCGTTGGCTCACTCCTATAATCCCA
GCATTTTGGAAGGCCAAGGTGGGCAGATTGCTTGAGCCCAGGAGTTTAAG
ACCAGACTGGACAACATGGCAAAACCCTGTCTCTACCAAAAAAAAAAAAA
AATTAGCAGGGTGTGGTGGTGCACACCTGCAGTCCCAGCTACTCAGGAGG
CAAGCTTGTATTCCTAGCTACTTAGGAGGATAGTTTGAGCCCAGGAAGTC
AAGGCTGCAGTGAGCATGATCCTGCCATTGCACTCCAGCCAGAGCAAAAA
AGAGAGCGAAACCCCATCTCAAAAAAAAGGGAAGATTTAGCTATGTTGGA
CTTACCTGTCCTCATGGAGCTGAATAATGGCCACCCCTCCAGGTAGGGCC
TGAACTCTACCTTTGCCAGAGTCCCCTCCACTCCCTGTTGGTCTTAGACA
ATGAAACTGAGTGTTAGTAGCTATTTACCACCAAGCTCATGCTTGTTGTT
CTTATAATAAAGATAAATGGTTTAATAAATGGTATGATAAAGAAAATTAT
ATTATGGTATTATACCATTTAATAAATGGTATAATAAAGAAAATGGTTTT
TTGCACCCACATTTCCATTAAAAAGTGAGAAAATTAAAGATACCTGAGGA
TGGCAGAGTGTTTGATGAAAGATAGGGAAATGTTGGCCAGGCACCGTGGC
TCACACCTGTAATCCCAGCAGTTTAGGAGGCCGGGGCAGGCGGATCACAA
GGTCAGGAGTTCAAGATCAGCCTGGCCAACATAGTGAAACCCCGTCTCTA
CTAAAAATACAAAAAATTAGCCGGGAGTGGTGGCAGGTGCCTACAATCCC
AGCTACTCGGAAGGCTGAATGGCGCGATCTCAGCTCATTGCAACCTCTGC
CTCCCAGGTTCAAGCCATTCTTCTGCCTTAGCCTCCCTAGTAGCTGGGAT
TACAGGCGCCTGCCACCATGCCTAGCTAATTTTTATATTTTTAGTAGACG
TGGGGTTTCGCCATGTTGGCCAGGCTGGTCTCAAACTCCTGACCTTGGGT
GATCTGCCCGCCTCGGTCTCCCAAACTGCTGGGATTACAGGAGTGAGCCA
CAGTGCCCGGCCTCTAATTTTTATTTTTAATTTTTTTAATTTTTATTTTT
TTAATTTTTATTTTATTTATTTTTTGTAATTTTTAAAATATACAAAAAAA
GGGCCGGGTGTGGTGGCTCACGCCTGTAATCCCAGCACTTTTTGGGAGGC
TGAGGTGGGTGGATCACGAGGTCAGGAGATCGAGACCATCCTGGCTAACA
TGGTGAAACCCTGTCTCTACTAAAAATATAAAAAAATTAGCCGGGCCTGG
TGGCAGGTGCCTGTAGTCCCAGCTACTCGGGAGGCTGAGACAGGAGAATG
GCGTGAACTCGAGAGGTGGAGCTTGCAGTGAGCCAAGATCGCACCACTAC
ACTCCAGCCTGGGCGACAGAGTGAGACTTCATCTCAAAAAAAAAAAAAAT
TATATATATATATATACATATATATATGCAAACAAAGAGCATCTGAGTCA
TAATAATGTAAATCTATCACCTGACTGACCTGCTGCCACACCTCATGATC
TCATCTGATCCCCACACTCCTTCTCTTTGGGATACTGTGTACAGCCATAG
CGTGGGTGAACTTTGTATTCCTATCCTCCCCATTTTTGTTATTTTATTTT
ATTTCTTATTTATTTGAGACAGAGTCTCACTCTGTCATCCAGACTGTAAT
GCAGTGGCCTGATCTCGGCTCACTGCAACCTCCACCTCCCGGTTTCAAGC
GAATCTCCTGCCTCAGCCTCCTAAGTAGCTGGGACCTACAGGCACACACC
ACCACGCCCAGCTAATTTTTGCATTTTTAATAGAGACGGGGTTTCACCGT
GCTGGGCAGGCTGGTCTCGAACTCCTGACCTCAGGTGATTTGCCCACCTC
AGCCTTCCAAATTGTTAGGATTACAGGCATGAGTCACTGTGCCCGGCCTC
CTCCCCATTTTATAACAAGGGAAATGGAGGCCCAGAATGGTTAAGTAAAC
CCACCCAGGGCTAGCTGAGAATTAGCAACAGAGAACTGGGAGTAGAATTT
GTTCCCTGGCCCTTTGCTGTTTCTATTATAAGCCACCCAGTCTTAGATTT
TCTGTTACCTTATAATTAATGACTCAAATGCAGTTTCTGAGTGAGAAACA
CAAGTCCCAAACACTCTTTAAAGAGGCATAAAGATGTATCTTGTTGTTTT
CTTTTGTTTGAGACAAGGCCTGGCTCTATTGCCCAGGCTGGAGTGTGGTG
ACATGATCTTGGCTCACTGCAACATCTGTCTCCTGGGCTCAAGCCATCAT
CCCACCTCAGCCTCCTGAGTAGCTGGAACTACAGGAGCGCGCCCCCACAC
CTGGGTAATTTTTCTATTTTTTGTAGAGATGGGGTTTTGCCATGTTGCCC
AGGCTGGTCTCGAACTCCTGAGCTCAAGTGATCCACCCATCTTGGCCTCC
CAAAGTGCTGGGATTACAGGCGTGAGCCACTGTGCCCAGCCTCTTGTTGA
CTGTAGGACCACAAGTTGGCGGCTAACTTAGAGCAATGTTTGGCACACAG
GAAGCACTCATTAAATATTGACATTATTGTAGTTATTTTAATAGCCCAGC
ATTGCACTTTTAGGTCTTTCAGCTTTCAGTGATGATCAGTTGATAATTGA
TGATCTGGTGGAGTGGTTCTTAATGGTAGAGTTGGGGGCAATTTTACACT
CCCTTACACCCCACTAATCTTCCCCCCAACCCAATGGTAAAGCTATTGCA
CAGTACTTGGCAATGTCTAGAGACAATTTTGGTTGTCACAGCCTGGGGGG
AAGGTGCTACTGGCATCTAGTGGGTAGAGGCTAAGGATGCTGCTTAATTT
TTTTTTTTTTTGAGACAAGAGTTTCACTCTAGTTGCCCAGGCACAGAACA
GCTTCACAGAAGCTGTTAATGCACAGAATAGCTTCCTACAAAAAAGCATT
ACCTGGCCCAAAATGTCATTAGCTACCAGGCTGAGAAACCTGCCCTCCTA
GGCCGGGCGCTGTGGCTCACGCCCGTAATCTCAGCATATTGGGAGGCCGA
GGTGGGCGGATCCTGAGGTCAGGAGTTCGAGACCACCTGGACCAACACGG
AGAAACCCAGTCTCTACCAAAAATACAAAATTAGCCGGGCATGGTGGCAC
ATGCTTGTAATCCCAGCTACTCGGGAGGCTGAGGCAGGAGAACCGCTTGA
ACACAGAGGCAGAGATTGTGGTGAGCCGAGATCACACCATTGCACTCCAG
CCTGGGCAACTAGAGCGAAACTCTTGTCTCAAAAAAAAAAAAAAAAAAAA
AAACAGGGAAAGAAAAGAAAGGAAACCTGCCCTCCTATCATAGGATAATC
CCATTTCCTCCTGTCTAAAGAGACGCCTACTTAGTCATCCTGGGTGACTG
CATCAGGGAGGTAGATTTTGGAGTCTGAAAGGCTGGGTTCTGTCACTTTG
TTACAGTGCCTCTGGTGCAAAGAAAGCATTTTAAAAACCCTGTACAATTA
AAAAATTGAATTTAATTACTTTGTGTAACTTTGAATAATTCACAGAAGTC
TGAACTTCTTTATCCTGTCCTGTAAAATGGAGGTAAAAAGCCCTTGGCCG
AGAGCTGTTTTGAGGAAAAACTGAAATAACATTGGTAAAGTGTCGCACAG
TACTTGGCACACAGCAGCCCCTCGACAAACATTAGCTTTCTTTCCCTTTC
TTGTCGGTTTCTTCCTCTCCAAACCCGCGTGTTGCTTTTCTTTTTAATTA
TTTTTCTGTAGCCCTCCTTTGCGGCCACAAACTCGCTTTCTAACCCAGGT
TCAGCCCTTTTATTGGCTGAGTGACCTTGTGCAAGTCACTTTTCCCCTGT
AGGCCTCGGTTTATTCTCCGTAAAATCAGAAAGTTGGCCTCCGATCTCCA
AGCACGCTTTTCACGACGAAGTGGGACTGTTAAGTTTACAGAGCTGCTTT
CCTCCCCCGGGACTGATGGTACGGTCCCCGGGCGGCTCCCCACCCATCTG
TCGCAGACCTTGGTACAGGCCCAGGGGGCCCTCGGCGGCCTCTCCGGGCT
GCCCTTGCCCCCTGCCGAGTTCCGGGCCCGTGTCTGCGCAAGCTGATTGG
CTGGAGCGGTGCCCGGGCTGCGCGGCTATAGGTGAGCCCAGGAGGGGACG
GGCGGGGCGGGCCGGAGGCCCGCCCCCTCCCCCCTGGGTCGGACGCTGAG
CGGAGCCGCGGGCGGGAGGGCGGACGGACCGACTGACGGTAGGGACGGGA
GGCGAGCAAGATG

28. PD-1. Programmed cell death protein 1 (PD-1) is also known as CD279 (cluster of differentiation 279). This gene encodes a cell surface membrane protein of the immunoglobulin superfamily. This protein is expressed in pro-B cells and is thought to play a role in their differentiation. PD-1 has two ligands, PD-L1 and PD-L2. PD-L1 protein is upregulated on macrophages and dendritic cells (DC) in response to LPS and GM-CSF treatment, and on T cells and B cells upon TCR and B cell receptor signaling.

Monoclonal antibodies blocking PD-1 may overcome immune resistance and boost the immune system are being developed for the treatment of cancer (Weber 2010, Semin. Oncol. 37 (5): 430-9). Nivolumab, a representative antibody, produced complete or partial responses in non-small-cell lung cancer, melanoma, and renal-cell cancer, in a clinical trial with a total of 296 patients; colon and pancreatic cancer did not have a response (Topalian et al., 2012: N Engl J Med 2012; 366:2443-2454). In HIV, drugs targeting PD-1 may augment immune responses and/or facilitate HIV eradication.

Protein: PD-1 Gene: PDCD1 (Homo sapiens, chromosome 2, 242792033-242801058 [NCBI Reference Sequence: NC_{—}000002.11]; start site location: 242800990; strand: negative)

Gene Identification

| | |
|---|---|
| GeneID | 5133 |
| HGNC | 8760 |
| HPRD | 02590 |
| MIM | 600244 |

Targeted Sequences

| | | Relative |
|---|---|---|
| | | upstream |
| | | location |
| | | to gene |
| Sequence | | start |
| ID No: | Sequence (5′-3′) | site |
| 7297 | TGCCGCCTTCTCCACTGCTCAGGCG | 23 |
| 7316 | ACCGCCTGACAGCTGGCGCGGCTGCCTGGC | 1061 |
| 7379 | CTGCGAGGCGCGGCCACGGCG | 1171 |
| 7396 | CGAGGAGGAAAGGCAGGCGGAGTCCG | 3395 |
| 7397 | CAGCGAAGCTGCAGAACGTCCCCATCACCACG | 4268 |
| 7439 | CGACAGCCGTGGGAAGGTGCAGTACG | 4388 |
| 7440 | CGGGATTCCCTGGAGATGCCTCCAGCGCG | 4422 |
| 7466 | AGGCGGTCCCAGGGCTCAGGTGTGGG | 2229 |
| 7498 | GCGTGCACCCCGTGGCCAGCTC | 3813 |
| 7526 | CAACGTACACGCAATCCACAAC | 2832 |

Target Shift Sequences

| | | Relative |
|---|---|---|
| | | upstream |
| | | location |
| | | to gene |
| Sequence | | start |
| ID No: | Sequence (5′-3′) | site |
| 7297 | TGCCGCCTTCTCCACTGCTCAGGCG | 23 |
| 7298 | GCCGCCTTCTCCACTGCTCA | 24 |
| 7299 | CCGCCTTCTCCACTGCTCAG | 25 |
| 7300 | CGCCTTCTCCACTGCTCAGG | 26 |
| 7301 | GTGCCGCCTTCTCCACTGCT | 22 |
| 7302 | AGTGCCGCCTTCTCCACTGC | 21 |
| 7303 | GAGTGCCGCCTTCTCCACTG | 20 |
| 7304 | AGAGTGCCGCCTTCTCCACT | 19 |
| 7305 | CAGAGTGCCGCCTTCTCCAC | 18 |
| 7306 | CCAGAGTGCCGCCTTCTCCA | 17 |
| 7307 | ACCAGAGTGCCGCCTTCTCC | 16 |
| 7308 | CACCAGAGTGCCGCCTTCTC | 15 |
| 7309 | CCACCAGAGTGCCGCCTTCT | 14 |
| 7310 | CCCACCAGAGTGCCGCCTTC | 13 |
| 7311 | CCCCACCAGAGTGCCGCCTT | 12 |
| 7312 | GCCCCACCAGAGTGCCGCCT | 11 |
| 7313 | AGCCCCACCAGAGTGCCGCC | 10 |
| 7314 | CAGCCCCACCAGAGTGCCGC | 9 |
| 7315 | GCAGCCCCACCAGAGTGCCG | 8 |
| 7316 | ACCGCCTGACAGCTGGCGCGGCTGCCTGGC | 1061 |
| 7317 | CCGCCTGACAGCTGGCGCGG | 1062 |
| 7318 | CGCCTGACAGCTGGCGCGGC | 1063 |
| 7319 | GCCTGACAGCTGGCGCGGCT | 1064 |
| 7320 | CCTGACAGCTGGCGCGGCTG | 1065 |
| 7321 | CTGACAGCTGGCGCGGCTGC | 1066 |
| 7322 | TGACAGCTGGCGCGGCTGCC | 1067 |
| 7323 | GACAGCTGGCGCGGCTGCCT | 1068 |
| 7324 | ACAGCTGGCGCGGCTGCCTG | 1069 |
| 7325 | CAGCTGGCGCGGCTGCCTGG | 1070 |
| 7326 | AGCTGGCGCGGCTGCCTGGC | 1071 |
| 7327 | GCTGGCGCGGCTGCCTGGCT | 1072 |
| 7328 | CTGGCGCGGCTGCCTGGCTC | 1073 |
| 7329 | TGGCGCGGCTGCCTGGCTCC | 1074 |
| 7330 | GGCGCGGCTGCCTGGCTCCG | 1075 |
| 7331 | GCGCGGCTGCCTGGCTCCGA | 1076 |
| 7332 | CGCGGCTGCCTGGCTCCGAG | 1077 |
| 7333 | GCGGCTGCCTGGCTCCGAGA | 1078 |
| 7334 | CGGCTGCCTGGCTCCGAGAG | 1079 |
| 7335 | GGCTGCCTGGCTCCGAGAGA | 1080 |
| 7336 | GCTGCCTGGCTCCGAGAGAC | 1081 |
| 7337 | CTGCCTGGCTCCGAGAGACA | 1082 |
| 7338 | TGCCTGGCTCCGAGAGACAC | 1083 |
| 7339 | GCCTGGCTCCGAGAGACACT | 1084 |
| 7340 | CCTGGCTCCGAGAGACACTC | 1085 |
| 7341 | CTGGCTCCGAGAGACACTCG | 1086 |
| 7342 | TGGCTCCGAGAGACACTCGG | 1087 |
| 7343 | GGCTCCGAGAGACACTCGGC | 1088 |
| 7344 | GCTCCGAGAGACACTCGGCC | 1089 |
| 7345 | CTCCGAGAGACACTCGGCCC | 1090 |
| 7346 | TCCGAGAGACACTCGGCCCG | 1091 |
| 7347 | CCGAGAGACACTCGGCCCGG | 1092 |
| 7348 | CGAGAGACACTCGGCCCGGC | 1093 |
| 7349 | GAGAGACACTCGGCCCGGCT | 1094 |
| 7350 | AGAGACACTCGGCCCGGCTC | 1095 |
| 7351 | GAGACACTCGGCCCGGCTCT | 1096 |
| 7352 | AGACACTCGGCCCGGCTCTG | 1097 |
| 7353 | GACACTCGGCCCGGCTCTGA | 1098 |
| 7354 | ACACTCGGCCCGGCTCTGAA | 1099 |
| 7355 | CACTCGGCCCGGCTCTGAAG | 1100 |
| 7356 | ACTCGGCCCGGCTCTGAAGG | 1101 |
| 7357 | CTCGGCCCGGCTCTGAAGGG | 1102 |
| 7358 | TCGGCCCGGCTCTGAAGGGA | 1103 |
| 7359 | CGGCCCGGCTCTGAAGGGAA | 1104 |
| 7360 | GGCCCGGCTCTGAAGGGAAA | 1105 |
| 7361 | GCCCGGCTCTGAAGGGAAAA | 1106 |
| 7362 | CCCGGCTCTGAAGGGAAAAC | 1107 |
| 7363 | CCGGCTCTGAAGGGAAAACA | 1108 |
| 7364 | CGGCTCTGAAGGGAAAACAT | 1109 |
| 7365 | AACCGCCTGACAGCTGGCGC | 1060 |
| 7366 | AAACCGCCTGACAGCTGGCG | 1059 |
| 7367 | GAAACCGCCTGACAGCTGGC | 1058 |
| 7368 | AGAAACCGCCTGACAGCTGG | 1057 |
| 7369 | TAGAAACCGCCTGACAGCTG | 1056 |
| 7370 | CTAGAAACCGCCTGACAGCT | 1055 |
| 7371 | GCTAGAAACCGCCTGACAGC | 1054 |
| 7372 | GGCTAGAAACCGCCTGACAG | 1053 |
| 7373 | AGGCTAGAAACCGCCTGACA | 1052 |
| 7374 | GAGGCTAGAAACCGCCTGAC | 1051 |
| 7375 | CGAGGCTAGAAACCGCCTGA | 1050 |
| 7376 | GCGAGGCTAGAAACCGCCTG | 1049 |
| 7377 | AGCGAGGCTAGAAACCGCCT | 1048 |
| 7378 | AAGCGAGGCTAGAAACCGCC | 1047 |
| 7379 | CTGCGAGGCGCGGCCACGGCG | 1171 |
| 7380 | TGCGAGGCGCGGCCACGGCG | 1172 |
| 7381 | GCGAGGCGCGGCCACGGCGA | 1173 |
| 7382 | CGAGGCGCGGCCACGGCGAG | 1174 |
| 7383 | TCTGCGAGGCGCGGCCACGG | 1170 |
| 7384 | GTCTGCGAGGCGCGGCCACG | 1169 |
| 7385 | TGTCTGCGAGGCGCGGCCAC | 1168 |
| 7386 | ATGTCTGCGAGGCGCGGCCA | 1167 |
| 7387 | GATGTCTGCGAGGCGCGGCC | 1166 |
| 7388 | TGATGTCTGCGAGGCGCGGC | 1165 |
| 7389 | ATGATGTCTGCGAGGCGCGG | 1164 |
| 7390 | GATGATGTCTGCGAGGCGCG | 1163 |
| 7391 | AGATGATGTCTGCGAGGCGC | 1162 |
| 7392 | AAGATGATGTCTGCGAGGCG | 1161 |
| 7393 | AAAGATGATGTCTGCGAGGC | 1160 |
| 7394 | CAAAGATGATGTCTGCGAGG | 1159 |
| 7395 | TCAAAGATGATGTCTGCGAG | 1158 |
| 7396 | CGAGGAGGAAAGGCAGGCGGAGTCCG | 3395 |
| 7397 | CAGCGAAGCTGCAGAACGTCCCCATCACCACG | 4268 |
| 7398 | AGCGAAGCTGCAGAACGTCC | 4269 |
| 7399 | GCGAAGCTGCAGAACGTCCC | 4270 |
| 7400 | CGAAGCTGCAGAACGTCCCC | 4271 |
| 7401 | GAAGCTGCAGAACGTCCCCA | 4272 |
| 7402 | AAGCTGCAGAACGTCCCCAT | 4273 |
| 7403 | AGCTGCAGAACGTCCCCATC | 4274 |
| 7404 | GCTGCAGAACGTCCCCATCA | 4275 |
| 7405 | CTGCAGAACGTCCCCATCAC | 4276 |
| 7406 | TGCAGAACGTCCCCATCACC | 4277 |
| 7407 | GCAGAACGTCCCCATCACCA | 4278 |
| 7408 | CAGAACGTCCCCATCACCAC | 4279 |
| 7409 | AGAACGTCCCCATCACCACG | 4280 |
| 7410 | GAACGTCCCCATCACCACGG | 4281 |
| 7411 | AACGTCCCCATCACCACGGG | 4282 |
| 7412 | ACGTCCCCATCACCACGGGG | 4283 |
| 7413 | CGTCCCCATCACCACGGGGT | 4284 |
| 7414 | GTCCCCATCACCACGGGGTC | 4285 |
| 7415 | TCCCCATCACCACGGGGTCC | 4286 |
| 7416 | CCCCATCACCACGGGGTCCT | 4287 |
| 7417 | CCCATCACCACGGGGTCCTC | 4288 |
| 7418 | CCATCACCACGGGGTCCTCC | 4289 |
| 7419 | CATCACCACGGGGTCCTCCG | 4290 |
| 7420 | ATCACCACGGGGTCCTCCGG | 4291 |
| 7421 | TCACCACGGGGTCCTCCGGG | 4292 |
| 7422 | CACCACGGGGTCCTCCGGGT | 4293 |
| 7423 | ACCACGGGGTCCTCCGGGTG | 4294 |
| 7424 | CCACGGGGTCCTCCGGGTGC | 4295 |
| 7425 | CACGGGGTCCTCCGGGTGCC | 4296 |
| 7426 | ACGGGGTCCTCCGGGTGCCC | 4297 |
| 7427 | CGGGGTCCTCCGGGTGCCCT | 4298 |
| 7428 | GGGGTCCTCCGGGTGCCCTT | 4299 |
| 7429 | GGGTCCTCCGGGTGCCCTTG | 4300 |
| 7430 | GGTCCTCCGGGTGCCCTTGG | 4301 |
| 7431 | GTCCTCCGGGTGCCCTTGGC | 4302 |
| 7432 | TCCTCCGGGTGCCCTTGGCA | 4303 |
| 7433 | CCTCCGGGTGCCCTTGGCAA | 4304 |
| 7434 | CTCCGGGTGCCCTTGGCAAT | 4305 |
| 7435 | TCCGGGTGCCCTTGGCAATA | 4306 |
| 7436 | CCGGGTGCCCTTGGCAATAC | 4307 |
| 7437 | CGGGTGCCCTTGGCAATACA | 4308 |
| 7438 | ACAGCGAAGCTGCAGAACGT | 4267 |
| 7439 | CGACAGCCGTGGGAAGGTGCAGTACG | 4388 |
| 7440 | CGGGATTCCCTGGAGATGCCTCCAGCGCG | 4422 |
| 7441 | CCGGGATTCCCTGGAGATGC | 4421 |
| 7442 | TCCGGGATTCCCTGGAGATG | 4420 |
| 7443 | TTCCGGGATTCCCTGGAGAT | 4419 |
| 7444 | CTTCCGGGATTCCCTGGAGA | 4418 |
| 7445 | CCTTCCGGGATTCCCTGGAG | 4417 |
| 7446 | TCCTTCCGGGATTCCCTGGA | 4416 |
| 7447 | ATCCTTCCGGGATTCCCTGG | 4415 |
| 7448 | CATCCTTCCGGGATTCCCTG | 4414 |
| 7449 | GCATCCTTCCGGGATTCCCT | 4413 |
| 7450 | CGCATCCTTCCGGGATTCCC | 4412 |
| 7451 | ACGCATCCTTCCGGGATTCC | 4411 |
| 7452 | TACGCATCCTTCCGGGATTC | 4410 |
| 7453 | GTACGCATCCTTCCGGGATT | 4409 |
| 7454 | AGTACGCATCCTTCCGGGAT | 4408 |
| 7455 | CAGTACGCATCCTTCCGGGA | 4407 |
| 7456 | GCAGTACGCATCCTTCCGGG | 4406 |
| 7457 | TGCAGTACGCATCCTTCCGG | 4405 |
| 7458 | GTGCAGTACGCATCCTTCCG | 4404 |
| 7459 | GGTGCAGTACGCATCCTTCC | 4403 |
| 7460 | AGGTGCAGTACGCATCCTTC | 4402 |
| 7461 | AAGGTGCAGTACGCATCCTT | 4401 |
| 7462 | GAAGGTGCAGTACGCATCCT | 4400 |
| 7463 | GGAAGGTGCAGTACGCATCC | 4399 |
| 7464 | GGGAAGGTGCAGTACGCATC | 4398 |
| 7465 | TGGGAAGGTGCAGTACGCAT | 4397 |
| 7466 | AGGCGGTCCCAGGGCTCAGGTGTGGG | 2229 |
| 7467 | GGCGGTCCCAGGGCTCAGGT | 2230 |
| 7468 | GCGGTCCCAGGGCTCAGGTG | 2231 |
| 7469 | CGGTCCCAGGGCTCAGGTGT | 2232 |
| 7470 | TAGGCGGTCCCAGGGCTCAG | 2228 |
| 7471 | ATAGGCGGTCCCAGGGCTCA | 2227 |
| 7472 | GATAGGCGGTCCCAGGGCTC | 2226 |
| 7473 | AGATAGGCGGTCCCAGGGCT | 2225 |
| 7474 | CAGATAGGCGGTCCCAGGGC | 2224 |
| 7475 | GCAGATAGGCGGTCCCAGGG | 2223 |
| 7476 | AGCAGATAGGCGGTCCCAGG | 2222 |
| 7477 | AAGCAGATAGGCGGTCCCAG | 2221 |
| 7478 | GAAGCAGATAGGCGGTCCCA | 2220 |
| 7479 | CGAAGCAGATAGGCGGTCCC | 2219 |
| 7480 | CCGAAGCAGATAGGCGGTCC | 2218 |
| 7481 | CCCGAAGCAGATAGGCGGTC | 2217 |
| 7482 | CCCCGAAGCAGATAGGCGGT | 2216 |
| 7483 | ACCCCGAAGCAGATAGGCGG | 2215 |
| 7484 | CACCCCGAAGCAGATAGGCG | 2214 |
| 7485 | CCACCCCGAAGCAGATAGGC | 2213 |
| 7486 | CCCACCCCGAAGCAGATAGG | 2212 |
| 7487 | CCCCACCCCGAAGCAGATAG | 2211 |
| 7488 | ACCCCACCCCGAAGCAGATA | 2210 |
| 7489 | GACCCCACCCCGAAGCAGAT | 2209 |
| 7490 | GGACCCCACCCCGAAGCAGA | 2208 |
| 7491 | GGGACCCCACCCCGAAGCAG | 2207 |
| 7492 | TGGGACCCCACCCCGAAGCA | 2206 |
| 7493 | CTGGGACCCCACCCCGAAGC | 2205 |
| 7494 | CCTGGGACCCCACCCCGAAG | 2204 |
| 7495 | TCCTGGGACCCCACCCCGAA | 2203 |
| 7496 | GTCCTGGGACCCCACCCCGA | 2202 |
| 7497 | GGTCCTGGGACCCCACCCCG | 2201 |
| 7498 | GCGTGCACCCCGTGGCCAGCTC | 3813 |
| 7499 | CGTGCACCCCGTGGCCAGCT | 3814 |
| 7500 | GTGCACCCCGTGGCCAGCTC | 3815 |
| 7501 | TGCACCCCGTGGCCAGCTCA | 3816 |
| 7502 | GCACCCCGTGGCCAGCTCAT | 3817 |
| 7503 | CACCCCGTGGCCAGCTCATA | 3818 |
| 7504 | ACCCCGTGGCCAGCTCATAT | 3819 |
| 7505 | CCCCGTGGCCAGCTCATATC | 3820 |
| 7506 | CCCGTGGCCAGCTCATATCT | 3821 |
| 7507 | CCGTGGCCAGCTCATATCTA | 3822 |
| 7508 | CGTGGCCAGCTCATATCTAA | 3823 |
| 7509 | GGCGTGCACCCCGTGGCCAG | 3812 |
| 7510 | AGGCGTGCACCCCGTGGCCA | 3811 |
| 7511 | CAGGCGTGCACCCCGTGGCC | 3810 |
| 7512 | ACAGGCGTGCACCCCGTGGC | 3809 |
| 7513 | CACAGGCGTGCACCCCGTGG | 3808 |
| 7514 | CCACAGGCGTGCACCCCGTG | 3807 |
| 7515 | ACCACAGGCGTGCACCCCGT | 3806 |
| 7516 | GACCACAGGCGTGCACCCCG | 3805 |
| 7517 | GGACCACAGGCGTGCACCCC | 3804 |
| 7518 | GGGACCACAGGCGTGCACCC | 3803 |
| 7519 | TGGGACCACAGGCGTGCACC | 3802 |
| 7520 | CTGGGACCACAGGCGTGCAC | 3801 |
| 7521 | GCTGGGACCACAGGCGTGCA | 3800 |
| 7522 | AGCTGGGACCACAGGCGTGC | 3799 |
| 7523 | TAGCTGGGACCACAGGCGTG | 3798 |
| 7524 | GTAGCTGGGACCACAGGCGT | 3797 |
| 7525 | AGTAGCTGGGACCACAGGCG | 3796 |
| 7526 | CAACGTACACGCAATCCACAAC | 2832 |
| 7527 | AACGTACACGCAATCCACAA | 2833 |
| 7528 | ACGTACACGCAATCCACAAC | 2834 |
| 7529 | CGTACACGCAATCCACAACA | 2835 |
| 7530 | GTACACGCAATCCACAACAC | 2836 |
| 7531 | TACACGCAATCCACAACACA | 2837 |
| 7532 | ACACGCAATCCACAACACAT | 2838 |
| 7533 | CACGCAATCCACAACACATA | 2839 |
| 7534 | ACGCAATCCACAACACATAC | 2840 |
| 7535 | CGCAATCCACAACACATACA | 2841 |
| 7536 | CCAACGTACACGCAATCCAC | 2831 |
| 7537 | CCCAACGTACACGCAATCCA | 2830 |
| 7538 | CCCCAACGTACACGCAATCC | 2829 |
| 7539 | TCCCCAACGTACACGCAATC | 2828 |
| 7540 | ATCCCCAACGTACACGCAAT | 2827 |
| 7541 | AATCCCCAACGTACACGCAA | 2826 |
| 7542 | CAATCCCCAACGTACACGCA | 2825 |
| 7543 | ACAATCCCCAACGTACACGC | 2824 |
| 7544 | CACAATCCCCAACGTACACG | 2823 |
| 7545 | GCACAATCCCCAACGTACAC | 2822 |
| 7546 | TGCACAATCCCCAACGTACA | 2821 |
| 7547 | ATGCACAATCCCCAACGTAC | 2820 |
| 7548 | CATGCACAATCCCCAACGTA | 2819 |
| 7549 | ACATGCACAATCCCCAACGT | 2818 |
| 7550 | AACATGCACAATCCCCAACG | 2817 |

Hot Zones (Relative upstream location to gene start site)
1-1450
1850-2350
2750-3000
3100-3600
3650-4050
4100-5000

### Examples

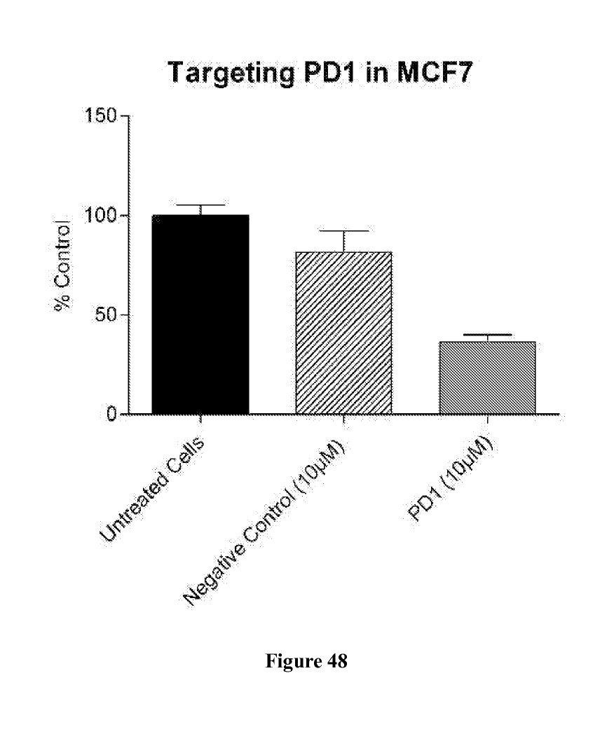

In FIG. 48, In MCF7 (human mammary breast cell line), PD1 (293) produced statistically significant (P<0.05) inhibition at 10 μM compared to the untreated and negative control values. The PD1 sequence PD1 (293) fits the independent and dependent DNAi motif claims.

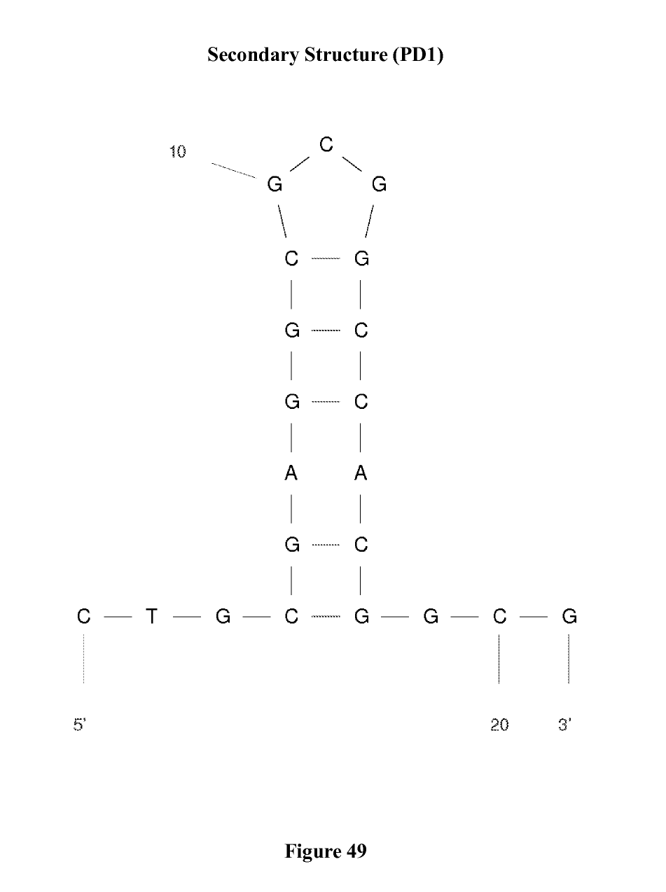

The secondary structure for PD1 (293) is shown in FIG. 49.

Genetic Code (5′ Upstream Region)

(SEQ ID NO: 11977)

ACCACAGCCTGGATGGCTCGGCACAGAGAGGAACGGGCTGCTGAAACACA
CGCGCTGGAGGCATCTCCAGGGAATCCCGGAAGGATGCGTACTGCACCTT
CCCACGGCTGTCGCTGGAATGAAACAGTGATGGGGGTGGAGGGCAGGCTC
GTGGCCACCAGCAGGGAGGTGGGTGTATTGCCAAGGGCACCCGGAGGACC
CCGTGGTGATGGGGACGTTCTGCAGCTTCGCTGTCAGGGGATGCAGGAGC
CTTCGCTTGTGCCCACATTGTGCAGCCTTTGATAGGCACACATTAGCCAG
AAACGGGGACTCAGGATGGGATCGAGGTGTCACATCAAAGTCATTAACCT
GGTTGTGACGTTGTCCTGTGGTTTTCCAAACTGTTATCATTCAGAGAGAC
TGAGCAGAGTGTATGAGGAAACATCTGTGTAATTTCTTACAACTGCAAGT
AAATCTACAATTATCTCAATTGTAAATGATACAATACTCAACCAAAACAT
ACAACCATCAGCCAGGTGTGGTGGCTCACGCCTGTAATCCCAACTCTTTG
GGAGTCCAAGGTGGGAGAATTGCTGGAGGCCCGGAGTTTGAGACCAGCCT
GGGTAACATAAAGAGACCTCCTCTGCCCCCACCCCAAATTCTACAAAAAA
AAAAAATGTTAGATATGAGCTGGCCACGGGGTGCACGCCTGTGGTCCCAG
CTACTCAGGAGGCTGAGGTCGGAGGATCGCTTGAGCCCAGGAGGTCGAGG
CTGCAGTGAGCCAAGATCACACCACTGCACTCCAGCCTGGGTGCAGAGCA
AGACCCTGTCTCTAAAAGAAAATAAACAGACAAAAACCACATACAACTTT
GCTTGTTGTAAATTATCTTTTAACTGAATGCCCTGGATTGAATCTGGCTG
CTGCCATCCCAGGGCCAGTGATTTGGATGGGGTATGACCCTCTGTGAGGA
AGGAGCAGGCGGTGGGGGAAGGGCCTGGGTGTCCAGGTTCCCTGGGAAGG
AAGGCTGAGAAAAGGAGATGGGGGAGGGGTGCGCAGGGCCGGCCAGCCAA
GGGCCCCTTAGCCCCATCTACCCTGCTCCCCGGACTCCGCCTGCCTTTCC
TCCTCGTGACAGAAGACAGTGGAAGCCTACTGGGTGGAAGGCACGGGCTT
AGGATGTGTGTGGGAGGAAAGTGTGTGTGCTGGGGAGCATGTATGTTTGG
GAGTTGTGTGTGTTGGAAATCGTGTGTTGGGGATTGTGTGTATATTGCAG
ATTTTGTATGTGTGTTGGGGATTGTGGTGTGTGGGTGTTGTAGATTGCGT
GTTGGGGATTGTGTTGGGGATTGTGTATGTGTTGGGGGTTGTGTGTGTGT
TGGGGATTGTGTGTGGGGGAGATTGTGTGTGTGTGCTGGGGATTGGGTGT
GTTGGGGATTGTGTGTGTGTTGAGGATTGTGTGTGGGGGAGATTCTGTGT
GTGTGCTGGGGATTGGGTGTGTTGGGGATTGTGTGTGTATTGGGAATTGT
GTGTGTGTTGAGGATTGTGTGTGTTGGGGATTTGTGTGTGTGTTGGGGAT
TCTGTGCATGTTGGGAGTTGTGTGTGAGTTGGGGACAATGTGTACAGAGG
ATTGTGTGTTGGAAATTTTGTGTGTGCGTTGGGAATTTTGTGTATGTGTT
GTGGATTGCGTGTACGTTGGGGATTGTGCATGTTGGGAATTTTGTGTGTG
TGTTGAGAATTGTGTGTGAGGGAATTGTGTGTGTTTGAGATTGTGTGTGT
ATTGGGAATTGTGTGTGTGTTGAGGATTGTGTGTGTTCTGAGGATTGTAT
GTGTTGGGAATTTTGTGTGTGTGTTGAGGATTGTGTGTGTTGGGGATTCT
ACGTATGTTGAAAGTTGTGTGTGTGTTGGGATTGTGTGTGTGTTGTGGAT
TGTGTGTGTTGGGAATTGTGTGTGTGTGTTGAGGATTGTGTGCAGGGGGA
TTGTGTGTGTTGGAGATTGTATGTGTTGGGAATTTTGTGTGTGTGTTGGG
GACTGTGTATGTTTTGGGGATTGTGTGTGTTGGGAATTTTGTGTGTGTGT
TGAGGATTGTGTGTGGGGGGATTGTGTGTGTTGGAGATTGTGTGTGTGTT
GGGGACTGTGTGTGTGTTGGGGACTGTGTGTGTTGGGGTGTGGTGTGTTG
GAAATCGTGTGTTGGGGACACCGTATGTGTTTGGGGGAGGGTGTCAATAA
GTGGTCTGGAGTGTGATATTGGGGTGCAGGCTCCATGAGTCCCCACCCCA
CACCTGAGCCCTGGGACCGCCTATCTGCTTCGGGGTGGGGTCCCAGGACC
CTGTAGGTTCAGCCTACTAGTCCAGGCCCAATGCCCAATGCCTGCATCCC
TGCAGGCCCTGTGCTCTCCAGGCTCAGACCCCTCGCAGCCCTGCAGACCC
TCCCTGGGTCCATGTGTCTCTTTGCAGGTGCTCCAGCGAGTAGCAATGTG
GAGAGACCATCAGGCAGCCCTGGCCTCAGTGGCCGCAGTCCCCTGGCTCC
ACGCTGGGCCCACCCCACCAGGTCTCCTCTCCCATGGCCCAGGGGCCTTC
AGTGGGACTGAGAGGAGGAGGGAAGGAGAGTGGGTGACAGGGAAGAACTG
CAGGGAGAGAGGAGAGGGGTGGGAGAAGGAGAAGGAAGGAAGGGGTAGGA
TGGAAGCTGGGTTTCTCCCTGTGCCCGCCCCCTACTCCAGGACATGTGTC
CAAGCCCTGGCAGGTGGAATTTTGGGGGCAGGGCCTTGGTGGTGAGGAGA
CCTTCCAGGGGTCTGATAGCATCTCCCATCTCAGAGCCCACCTCCTGGGC
CCAGCCTCCCCTCCAGCCCACACAGTGGCATTCCCAGTCCTCAGAGGACA
GCTTCGTCCCACAAAGCTCAGAGCCTTGAGGAAGGCCCACTGCTGCCCTG
GAACAGAGACAGCATTCAACAGAGGTTGGAACAAGGCTCTACAGGGCTGG
GGGCAGAGGGAGGTTCTGTCCAGAATCTGCCTTCAGGACAAGTACAGCCA
GCAGGGGCAGCTTAGCCACTTATCCACTGCCTGGGCGAGGCACAGGGCTA
TGGAGGCACCTACCAACCAACAGTTCTCCAGCCCCAGAGCCCCAGCCCCT
GAGGCACAAGGGTGGGTGTGCCAGGAGACAGTTGCTGCGGGCCACCTTAG
CTGTCTGGCAGCACAGTGGGTGCTGCCAGGCTCCCTGGGGGCCCCCCGCC
AAGCCCACCTGGCCAGCTGGGCCCCCCCCACCTCCCCACCAAGCCACCCA
CACAGCCTCACATCTCTGAGACCCGGGAGTGGCCCTTTGTTCATAAACGA
GAGCTTCCTCGCCGTGGCCGCGCCTCGCAGACATCATCTTTGATGCTCTT
TTTCCACTGTTTCGGTGCTTTAATGTTTTCCCTTCAGAGCCGGGCCGAGT
GTCTCTCGGAGCCAGGCAGCCGCGCCAGCTGTCAGGCGGTTTCTAGCCTC
GCTTCGGTTATTTTAAGCTGATGAGCCTGACGCATCTCATCACTAATATC
AGCAGTTTCATTTCTCCTGTTTTCCATTCGCTGTAATAAAATGCTCAGCA
CAGAATACAAGGAGATAAGCAAGCCATTTCACAAACGCCGGGCCGCCAGC
CAGGCCCAGGCACTGGACCCCCTGAACCACCCCACCCTGGCACGAGTGGG
CTGGAGGGCAGGGCCCCGGGGAAGAAGGTCAAGGCTGGAAGGGGAGGTCA
GCCTCACAGCCAGCCCCTGCCACCGCCCCAGCCCCCCCGTCAGGCTGTTG
CAGGCATCACACGGTGGAAAGATCTGGAACTGTGGCCATGGTGTGAGGCC
ATCCACAAGGTGGAAGCTTTGAGGGGGAGCCGATTAGCCATGGACAGTTG
TCATTCAGTAGGGTCACCTGTGCCCCAGCGAAGGGGGATGGGCCGGGAAG
GCAGAGGCCAGGCACCTGCCCCCAGCAGGGGCAGAGGCTGTGGGCAGCCG
GGAGGCTCCCAGAGGCTCCGACAGAATGGGAGTGGGGTTGAGCCCACCCC
TCACTGCAGCCCAGGAACCTGAGCCCAGAGGGGGCCACCCACCTTCCCCA
GGCAGGGAGGCCCGGCCCCCAGGGAGATGGGGGGGATGGGGGAGGAGAAG
GGCCTGCCCCCACCCGGCAGCCTCAGGAGGGGCAGCTCGGGCGGGATATG
GAAAGAGGCCACAGCAGTGAGCAGAGACACAGAGGAGGAAGGGGCCCTGA
GCTGGGGAGACCCCCACGGGGTAGGGCGTGGGGGCCACGGGCCCACCTCC
TCCCCATCTCCTCTGTCTCCCTGTCTCTGTCTCTCTCTCCCTCCCCCACC
CTCTCCCCAGTCCTACCCCCTCCTCACCCCTCCTCCCCCAGCACTGCCTC
TGTCACTCTCGCCCACGTGGATGTGGAGGAAGAGGGGGCGGGAGCAAGGG
GCGGGCACCCTCCCTTCAACCTGACCTGGGACAGTTTCCCTTCCGCTCAC
CTCCGCCTGAGCAGTGGAGAAGGCGGCACTCTGGTGGGGCTGCTCCAGGC
ATG

29. BCL2. Bcl-2 (B-cell lymphoma 2) is the founding member of the Bcl-2 family of apoptosis regulator proteins encoded by the BCL2 gene that was first described in chromosomal translocations involving chromosomes 14 and 18 in follicular lymphomas (Tsujimoto et al. Science 226 (4678): 1097-99). The dysregulation of cell death is a defining characteristic of malignant cells and BCL-2 protein plays a key and central role. BCL-2 confers an anti-apoptotic phenotype that contributes to the genesis of hematopoietic and lymphatic cancers. In many cases of diffuse large B-cell (DLBCL) and follicular lymphomas (FL), BCL2 overexpression is driven by the t(14,18) chromosomal rearrangement of the BCL2 oncogene. In chronic lymphocytic leukemia, impaired degradation of BCL2 mRNA causes continuous production of BCL2. The Bcl-2 gene has been implicated in a number of cancers, including melanoma, breast, prostate, chronic lymphocytic leukemia, skin, sarcoma, and lung carcinomas, as well as schizophrenia and autoimmunity. It is also thought to be involved in resistance to conventional cancer treatment and evidence also suggests that decreased apoptosis may play a role in the development of cancer.

Protein: BCL2 Gene: BCL2 (Homo sapiens, chromosome 18, 63123346-63319778 [NCBI Reference Sequence: NC_{—}000018.10]; start site location: 63318666; strand: negative)

Gene Identification

| | |
|---|---|
| GeneID | 596 |
| HGNC | 990 |
| MIM | 151430 |

Targeted Sequences

| | | | Relative |
|---|---|---|---|
| | | | upstream |
| | | | location to |
| Sequence | Design | | gene start |
| ID | ID | Sequence (5′-3′) | site |
| 13682 | | TGTCCACCTGAACACCTAGTCC | 2388 |

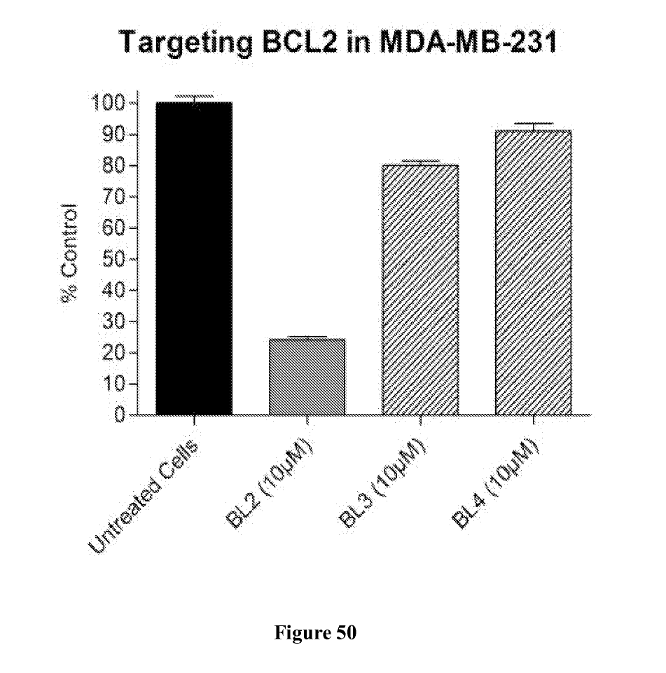

In FIG. 50, In MDA-MB-231 (human breast cell line), BL2 at 10 μM showed increased inhibition compared to BL3 and BL4 (10 μM). The BL2 (structure shown below) fits the independent and dependent DNAi motif claims. Both BL3 and BL4 contained a single mismatched base meaning neither sequence had 100% homology to its complementary strand. This demonstrates that many times even a single mismatch to the complementary strand decreases the inhibitory effects of a DNAi oligonucleotide. The mismatches for BL3 and BL4 are noted below with the mismatched letter highlighted and bolded. It should also be noted that a 20-mer version of BL2 demonstrated similar significant inhibition (data not shown) as the 24-mer version of BL2 shown in FIGS. 50, 51, and 52.

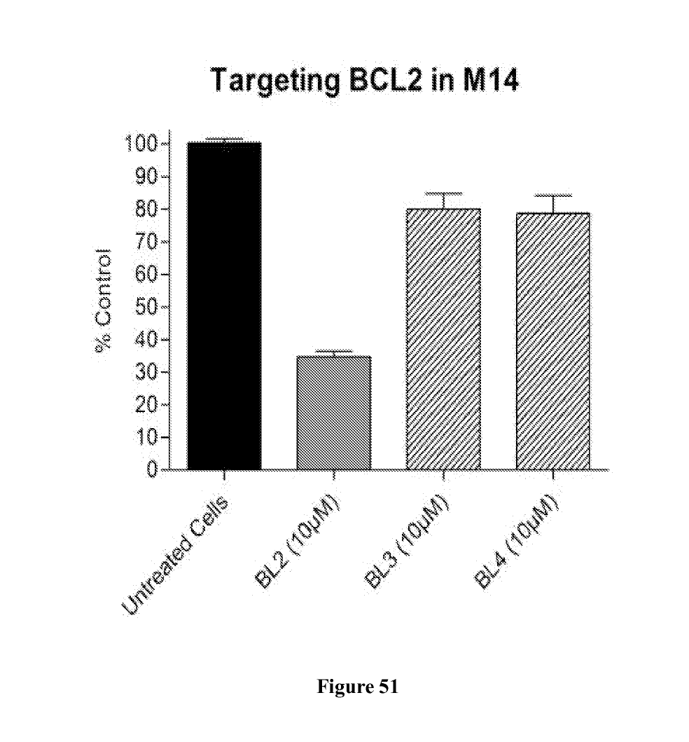

In FIG. 51, M14 (human melanoma cell line), BL2 at 10 μM showed increased inhibition compared to BL3 and BL4 (10 μM). The BL2 (structure shown below) fits the independent and dependent DNAi motif claims. Both BL3 and BL4 contained a single mismatched base meaning neither sequence had 100% homology to its complementary strand. This demonstrates that many times even a single mismatch to the complementary strand decreases the inhibitory effects of a DNAi oligonucleotide. The mismatches for BL3 and BL4 are noted below with the mismatched letter highlighted and bolded. It should also be noted that a 20-mer version of BL2 demonstrated similar significant inhibition (data not shown) as the 24-mer version of BL2 shown in FIGS. 50, 51, and 52.

BL3: ACCGGCGCTCGGCGCGCGGA (SEQ ID NO: 13825)(needed to have a G in place of the C for 100% homology)

BL4: GACGCGCCGGGCCGGGCGGA (SEQ ID NO: 13826) (needed to have an A in place of the C for 100% homology)

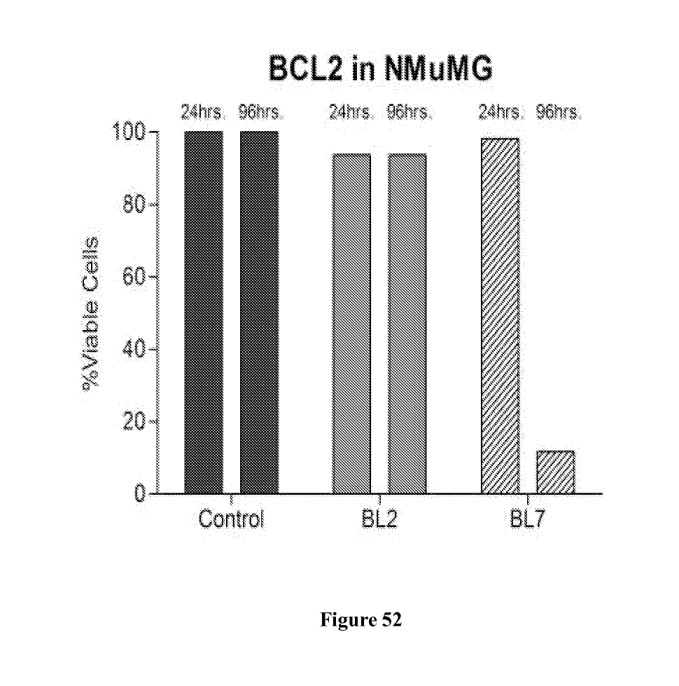

In FIG. 52, as a counter screen to test for nonspecific toxicity, BL2 and BL7 were tested at 10 μM in NMuMG (a normal murine mouse mammary gland cell line) and measured at 24 and 96 hours post exposure. As would be expected, BL2 has no cytotoxicity against a normal, nontumorigenic mouse cell line because it was designed for homology with the human genome and only has a maximum of 67% homology across the entire mouse genome. BL7, however, has approximately 90% homology across the entire mouse genome. This demonstrates that duplication and high overlap with non-targeted regions of the genome leads to non-specific cytotoxicity.

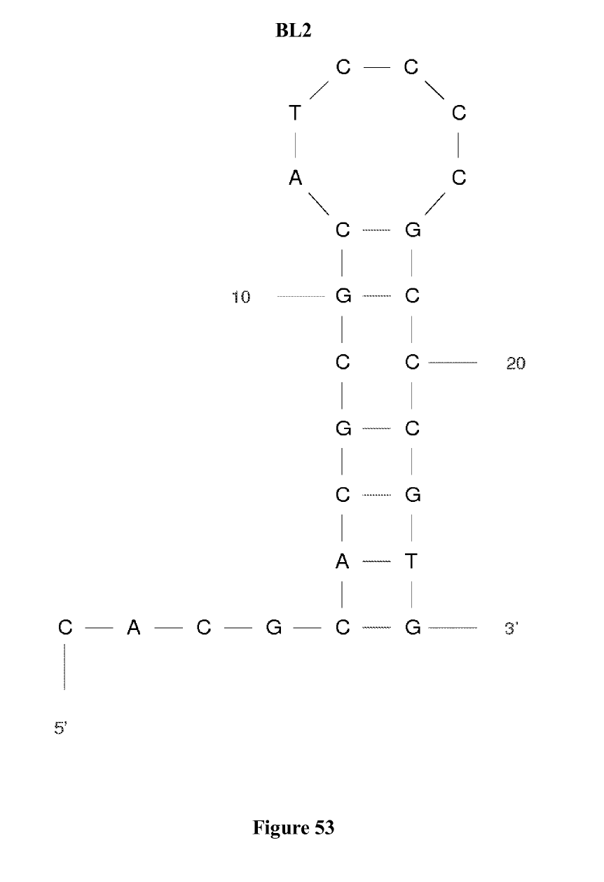

The secondary structure for BL2 are shown in FIG. 53. Sequence 302 (BL2) is shown in FIG. 53.

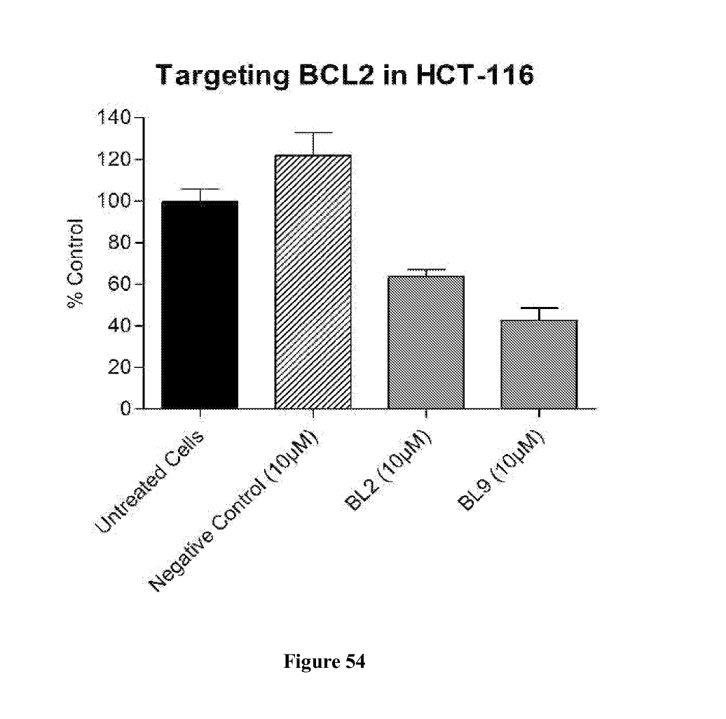

In FIG. 54, In HCT-116 (human colorectal carcinoma), BL9 produced statistically significant (P<0.05) inhibition at 10 μM compared to the untreated and negative control values. The BCL2 sequence BL9 will not form a secondary structure under physiological conditions.

Genetic Code (5′ Upstream Region)

(SEQ ID NO: 13683)

CCTCCCAAAGTGCTGAGATTACAGGCATGAGCAACCACACCTGGCCGATA
CATACCTATATTAAACATTAGTATGTTCATGTTAGAATAATGTACCTTTT
GAATTTCATAAACTTGGAGAATATTTATATTGATGATGGATGAAAGAACT
TTCTTGGATGGATGAAGAGAGTAACGCTGTGAAACAACCAGCAGGTGGCG
AAAACTGGCAATCAAAAGCTTTTTGTTTGGTGGCCTGGGGAATGAAGACG
GAAAGAAAACACAGGCCATTCAGACTCTTGATACAATCTCCATTCCCTGC
ATCTTGTTTTTTTTCTCTTCCTGGTGCCACGCTACTTGTAGAATCCAACC
AGGTAAAGCTGCCAAAAGGGTCATCCATTGGCTCTTACAAGTAGAAAACA
TCTTGGAAAGTGAAAAGTCCACTGTGCATATGTTTGTAAGGTTGTTGGAA
GGTCTCAGGCATAGATCTAGGATTCAAATCCAGTTACTCCTGCCTCAGGC
TGGTGTTCCCTCCCCCACCTCAGCATTGCCCAAAGACGAGTAGTCAATGT
CACATACTTCCTGGGAATACTTGCCCTTATGCTTTAAAATGGAATCTAAT
AAACATGGAATCTGAACGCAGGAATGGTTTCACTCTTTCATTTGAAAGAG
TATCTAGAACATTCCCAGGGAAAATATAACCCCTAGCCAAAGACTGCAAT
ACAGACCTGTCTCAAGACTGATTATAGCCAAGATGCCACATAAGGAATCA
GTCTGGGAAAATCCATAGAGTGAGGCTCTGTGGGAGCAAAGGAAGACGAA
AATCAGTCAGCTTTTCTTTCTCTGGAAGTAGGGGATCCGTTTCCTTCTGG
CTGCCCCCTTTGCAGAAGTACAGTTTCTTTTGCAGGTTTGTCCTATCATT
TCCTCACTCATATGCTGAGTATTAGGAGCTTGAAGCCTTTCAATTCCTCT
TAGGTAATTTTGGGGCTTTAAAATACGCTTTCAAGATTTCTAAACCATAC
TGTTGTGCAATTGGTATGAATTTATGTGAGAACATTTATTCTAGGTCAAT
CTATACCCAGTGTCTATCCAGACCAAAACACCTCCCACGCGCATAAAAGG
GACTCTGTCCCAACCATCAGAAGGGCAAGAAGGAGGATCTCCTTTCATCC
CCTCTTGCCTGGATAAGAAATTTGTACCCAGGCCCCCATTCCTATGTGAG
AGAAGTTGGCTTGTTGGGCTGATGGGATACAATAAATGAAGAAATAAAAT
AAAAACACCCAAGAGAGATGGCAGTGCGTATAGTCCCAGCTATTCATGAG
GCTGAGGTGGGAGAATCCTTCGAGCCCAGAAGTTCGAGTCCAGCCTGGGC
AACATAGCAAAAGCCATCTCTTAAAAAAAAAAAAAAAAGGCCAACTAAGT
AAAAATTAAAAAAATCATAATTTGGTGTGCTTTTCTGGCTTTTTAAAGAA
TGTTTTGATTTTAGAGTAGGAATGAGACAAAATAAAGATGTCAGGCAGGG
CACAGTGGCTCATGTCTGTAATCCCAGCACTTTGGGAGGCTGAGGCAGGC
GGATAACGAGGTCAGGAGTTCGAGACCAGCCTGGCCAGTATGGCGAAACC
CCGTCTCTACTAAAAATACAAAAATTAGCAGAGCGTAGTGGCGTGCACGT
GTAATCCCAGCTACTCAGGAGGCTGAGGCAGAATTGCTTGAGCCTGGGAG
GCAGAGGTTGCAGTGAGCTGAGATCGCACCACTGCACACCAGCCTCCAAG
ATACAACAGAGCAAGACTCTGTCTCAAAAAAAAAAAAAAAGTCATAGCAT
ATTTGTACACATTGTAGTACTCATTTGTCATCTTTCTTGACCCCAATAAT
CCAGTGTCCCTATATATTTGCACTCGAGCCCTATTAAGTAAGCCGCTGTG
CTTCTAGAAGACCTTTTTCTTTTCTTGGTGCTTTGTCAAAGACTCTTGGA
GATAAAAATACACACGTGCAACTTGTTTGTCCTCTTGTCCTTTTTTGCTA
GGGGCTATTCATGCTGATTAATTTAAAACTGTCTGCTTGCGCGTACACAC
GTCTGCGAGTGTGAATGTGTATGTGTGTATCTATGTACCTCATTTGAGAA
AGTGCGGCCAACTAGGATTGGCTACGAGGCAAAGGTGGAGACCTTTAGGA
GCCCACCCACCCCAGCGTTAGGACGGTGGGCCTGAAAGTTACTATATGGA
AGTCCTCATCGTGTAGCACTAAACCAGTGTAAAAGGTGTTAGGGACAGAG
GGAAAACATTGACTTAAACTGTCGTAAAGCCCTTGATAAACCCCTTCCCT
GGAGCGCTGAGTTCTGCATGGCCTGGGCCACGGACTAGGTGTTCAGGTGG
ACACGGGCGGGGATGCGCGTGCGTGTGTAGTGCGCGGACACCTAGGAAGC
TACTTGAAAGTAAACACCACGCTCGGGGCGTCCCTAGACATTGCTTAAAA
CGTGCAGAGTCACCTGTCTTCACAGCAGGGCAGCGCTGAGGTCCCACTGC
TGGGGGCGGTGGGGGGCGGCATTGGCCTGGGTCTTCCCCCGGCGGCCGAG
CGCCGGTAACACAACGTGTGTGTGTGTAGGCGCGTGTACACACTCTCATA
CACGGCTAGAAAGGGTCCAGGCGACACACACACTCCCACATACACGGCTA
GAAAAGGTCCAGGCGAGACACACACACACACACACACACACACACACACT
CCACACACACTCACACGGCCAGAAAGGGTCCAGGCGGTTCCCGGCGCTTT
TCCAGCCCTTGTTTTCATGGCGCACCCTCCCGCCAGCCGCCCCCCTCCGC
ACTCCGTCGTCCGCCCGGCCCGGCCGCGTGCGGTTCCCCGGGAGCCCCCA
CCCCGTCGCGGACCCCAGCGACCACCAAGTCCGCACGCGGCCTGCCGCAG
GCCTGAGCAGAAGGCCCCGCGCACACCCACCGCGCCGCGGCCGCGCGGGA
GGCCTGTGCCGCCCGCGCCACCCACTGGCCGGGCCCCGCGGGCGCAGCGG
AGCGGGCGGGTGGCCGGCCCGGACGCGCCCTCCCCGGCCGCGGCCCCGCG
CGCCATGTGCCCCCGGCGGGACGCGCCACTCCCGGGCCTGCCGCGGCGCC
TTTAACCCGGGCCAGGGAGCGGGGCGGAGGGGGCGGTCGGGTGGCTCAGA
GGAGGGCTCTTTCTTTCTTCTTTTTTTGAATGAACCGTGTGACGTTACGC
ACAGGAAACCGGTCGGGCTGTGCAGAGAATGAAGTAAGAGGACAGGCACC
ACAGCCCCGCTCCCGCCCCCTTCCTCCCGCGCCCGCCCCTCCGCGCCGCC
TGCCCGCCCGCCCGCCGCGCTCCCGCCCGCCGCTCTCCGTGGCCCCGCCG
CGCTGCCGCCGCCGCCGCTGCCAGCGAAGGTGCCGGGGCTCCGGGCCCTC
CCTGCCGGCGGCCGTCAGCGCTCGGAGCGGGCTGCGCGGCGGGAGCTCCG
GGAGGCGGCCGTAGCCAGCGCCGCCGCGCAGGACCAGGAGGAGGAGAAAG
GGTGCGCAGCCCGGAGGCGGGGTGCGCCGGTGGGGTGCAGCGGAAGAGGG
GGTCCAGGGGGGAGAACTTCGTAGCAGTCATCCTTTTTAGGAAAAGAGGG
AAAAAATAAAACCCTCCCCCACCACCTCCTTCTCCCCACCCCTCGCCGCA
CCACACACAGCGCGGGCTTCTAGCGCTCGGCACCGGCGGGCCAGGCGCGT
CCTGCCTTCATTTATCCAGCAGCTTTTCGGAAAATGCATTTGCTGTTCGG
AGTTTAATCAGAAGAGGATTCCTGCCTCCGTCCCCGGCTCCTTCATCGTC
CCCTCTCCCCTGTCTCTCTCCTGGGGAGGCGTGAAGCGGTCCCGTGGATA
GAGATTCATGCCTGTGCCCGCGCGTGTGTGCGCGCGTGTAAATTGCCGAG
AAGGGGAAAACATCACAGGACTTCTGCGAATACCGGACTGAAAATTGTAA
TTCATCTGCCGCCGCCGCTGCCTTTTTTTTTTCTCGAGCTCTTGAGATCT
CCGGTTGGGATTCCTGCGGATTGACATTTCTGTGAAGCAGAAGTCTGGGA
ATCGATCTGGAAATCCTCCTAATTTTTACTCCCTCTCCCCGCGACTCCTG
ATTCATTGGGAAGTTTCAAATCAGCTATAACTGGAGAGTGCTGAAGATTG
ATGGGATCGTTGCCTTATGCATTTGTTTTGGTTTTACAAAAAGGAAACTT
GACAGAGGATCATGCTGTACTTAAAAAATACAAGTAAGTTCTCTGCACAG
GAAATTGGTTTAATGTAACTTTCAATGGAAACCTTTGAGATTTTTTACTT
AAAGTGCATTCGAGTAAATTTAATTTCCAGGCAGCTTAATACATTCTTTT
TAGCCGTGTTACTTGTAGTGTGTATGCCCTGCTTTCACTCAGTGTGTACA
GGGAAACGCACCTGATTTTTTACTTATTAGTTTGTTTTTTCTTTAACCTT
TCAGCATCACAGAGGAAGTAGACTGATATTAACAATACTTACTAATAATA
ACGTGCCTCATGAAATAAAGATCCGAAAGGAATTGGAATAAAAATTTCCT
GCATCTCATGCCAAGGGGGAAACACCAGAATCAAGTGTTCCGCGTGATTG
AAGACACCCCCTCGTCCAAGAATGCAAAGCACATCCAATAAAATAGCTGG
ATTATAACTCCTCTTCTTTCTCTGGGGGCCGTGGGGTGGGAGCTGGGGCG
AGAGGTGCCGTTGGCCCCCGTTGCTTTTCCTCTGGGAAGGATG

BCL2

Apoptosis also plays a very active role in regulating the immune system. When functional, apoptosis causes immune unresponsiveness to self-antigens via both central and peripheral tolerance. When defective, it may contribute to autoimmune diseases (Li et al., Clin. Dev. Immunol. 13 (2-4): 273-82 and reviewed by Tischner et al., Cell Death and Disease (2010) 1, e48), such as type 1 diabetes, manifested as aberrant T cell AICD and defective peripheral tolerance. Dendritic cells are the most important antigen presenting cells of the immune system such that their activity must be tightly regulated by such mechanisms as apoptosis and their lifespan may be controlled in part by BCL-2. Other inflammatory diseases include inflammatory bowel disease, psoriatic arthritis, lupus, heart disease, and Alzheimer's and schizophrenia.

Given its biological importance, BCL2 is a prime candidate for targeted therapies. Numerous approaches that block or modulate production of BCL2 at the DNA level (e.g., retinoids and histone deacetylase inhibitors), RNA level (targeted antisense oligonucleotides such oblimersen and SPC2996 or siRNA approaches), or the protein level (gossypol, obatoclax, ABT-737, ABT-263, ABT-199) have been reported and a few have entered clinical development.

30. CMYC. Myc (c-Myc) is a regulator gene that codes for protein that is a transcription factor. In the human genome, Myc is located on chromosome 8 and is believed to regulate expression of 15% of all genes (Gearhart et al., N Engl J Med 2007; 357:1469-1472). CMYC activates expression of many genes through binding on consensus sequences (Enhancer Box sequences (E-boxes)) and recruiting histone acetyltransferases (HATs). This means that CMYC is activated upon various mitogenic signals such as Wnt, Shh and EGF (via the MAPK/ERK pathway). By modifying the expression of its target genes, Myc activation results in numerous biological effects. CMYC has the capability to drive cell proliferation (upregulates cyclins, downregulates p21), but it also plays a very important role in regulating cell growth (upregulates ribosomal RNA and proteins), apoptosis (downregulates Bcl-2), differentiation and stem cell self-renewal. CMYC is a very strong proto-oncogene and it is very often found to be upregulated in many types of cancers. Myc overexpression stimulates gene amplification (Denis et al., Oncogene 6 (8): 1453-7), presumably through DNA over-replication.

It can also act as a transcriptional repressor. By binding Miz-1 transcription factor and displacing the p300 co-activator, it inhibits expression of Miz-1 target genes. In addition, myc has a direct role in the control of DNA replication (Dominguez-Sola et al., Nature 448 (7152): 445-51).

Mutated CMYC is found in many cancers, causing it to be constitutively expressed thereby driving the unregulated expression of many genes involved in cell proliferation. A common human translocation involving CMYC is t(8; 14) which is critical to the development of most cases of Burkitt's Lymphoma. Malfunctions in Myc have also been found in carcinoma of the cervix, colon, breast, lung and stomach (Prochownik, 2004; Expert Rev Anticancer Ther.; 4(2):289-302).

Because CMYC is part of a dynamic network whose members interact selectively with one another and with various transcriptional coregulators and histone-modifying enzymes, it is an attractive therapeutic target. Several approaches including small molecules, peptides, and oligonucleotide therapeutics have been pursued. However, knowledge of which pathway should be attacked (c-Myc transcription, translation, interaction with other myc network members, DNA binding and transcriptional activation) is crucial. Clinical efficacy will likely require intervention at several levels, perhaps in combination with traditional chemotherapeutic drugs or agents that target other oncoproteins (reviewed by Levens, 2010; Genes and Cancer 1: 547).

CMYC

Protein: CMYC Gene: CMYC (Homo sapiens, chromosome 8, 128748315-128753680 [NCBI Reference Sequence: NC_{—}000008.10]; start site location: 128748840; strand: positive)

Gene Identification

| | |
|---|---|
| GeneID | 4609 |
| HGNC | 7553 |
| MIM | 190080 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 7551 | | CGATGAGGGTATTAACTCTGGC | 335580 |
| 7552 | | CGGGGGTCCTCAGCCGTCCAGACC | 518 |
| 7602 | | CGCTTATGGGGAGGGTGGGGAGGG | 634 |
| 7603 | | CGGTGGGCGGAGATTAGCGAGAGA | 559 |
| 7606 | | GGCGCTTATGGGGAGGGTGGGGAGGG | 632 |
| 506 | | CCTGGCACGTGTCCCTGGTCAAG | 3482 |
| 507 | | CACGTGCGGCCTGTCAAGAGATGA | 5926 |

Target Shift Sequences

| | | Relative |
|---|---|---|
| | | upstream |
| Sequence | | location to gene |
| ID No: | Sequence (5′-3′) | start site |
| 7551 | CGATGAGGGTATTAACTCTGGC | 335580 |
| 7552 | CGGGGGTCCTCAGCCGTCCAGACC | 518 |
| 7553 | GGGGGTCCTCAGCCGTCCAG | 519 |
| 7554 | GGGGTCCTCAGCCGTCCAGA | 520 |
| 7555 | GGGTCCTCAGCCGTCCAGAC | 521 |
| 7556 | GGTCCTCAGCCGTCCAGACC | 522 |
| 7557 | GTCCTCAGCCGTCCAGACCC | 523 |
| 7558 | TCCTCAGCCGTCCAGACCCT | 524 |
| 7559 | CCTCAGCCGTCCAGACCCTC | 525 |
| 7560 | CTCAGCCGTCCAGACCCTCG | 526 |
| 7561 | TCAGCCGTCCAGACCCTCGC | 527 |
| 7562 | CAGCCGTCCAGACCCTCGCA | 528 |
| 7563 | AGCCGTCCAGACCCTCGCAT | 529 |
| 7564 | GCCGTCCAGACCCTCGCATT | 530 |
| 7565 | CCGTCCAGACCCTCGCATTA | 531 |
| 7566 | CGTCCAGACCCTCGCATTAT | 532 |
| 7567 | GTCCAGACCCTCGCATTATA | 533 |
| 7568 | TCCAGACCCTCGCATTATAA | 534 |
| 7569 | CCAGACCCTCGCATTATAAA | 535 |
| 7570 | CAGACCCTCGCATTATAAAG | 536 |
| 7571 | AGACCCTCGCATTATAAAGG | 537 |
| 7572 | GACCCTCGCATTATAAAGGG | 538 |
| 7573 | ACCCTCGCATTATAAAGGGC | 539 |
| 7574 | CCCTCGCATTATAAAGGGCC | 540 |
| 7575 | CCTCGCATTATAAAGGGCCG | 541 |
| 7576 | CTCGCATTATAAAGGGCCGG | 542 |
| 7577 | TCGCATTATAAAGGGCCGGT | 543 |
| 7578 | CGCATTATAAAGGGCCGGTG | 544 |
| 7579 | GCATTATAAAGGGCCGGTGG | 545 |
| 7580 | CATTATAAAGGGCCGGTGGG | 546 |
| 7581 | ATTATAAAGGGCCGGTGGGC | 547 |
| 7582 | TTATAAAGGGCCGGTGGGCG | 548 |
| 7583 | TCGGGGGTCCTCAGCCGTCC | 517 |
| 7584 | CTCGGGGGTCCTCAGCCGTC | 516 |
| 7585 | GCTCGGGGGTCCTCAGCCGT | 515 |
| 7586 | AGCTCGGGGGTCCTCAGCCG | 514 |
| 7587 | CAGCTCGGGGGTCCTCAGCC | 513 |
| 7588 | ACAGCTCGGGGGTCCTCAGC | 512 |
| 7589 | CACAGCTCGGGGGTCCTCAG | 511 |
| 7590 | GCACAGCTCGGGGGTCCTCA | 510 |
| 7591 | AGCACAGCTCGGGGGTCCTC | 509 |
| 7592 | CAGCACAGCTCGGGGGTCCT | 508 |
| 7593 | GCAGCACAGCTCGGGGGTCC | 507 |
| 7594 | AGCAGCACAGCTCGGGGGTC | 506 |
| 7595 | GAGCAGCACAGCTCGGGGGT | 505 |
| 7596 | CGAGCAGCACAGCTCGGGGG | 504 |
| 7597 | GCGAGCAGCACAGCTCGGGG | 503 |
| 7598 | CGCGAGCAGCACAGCTCGGG | 502 |
| 7599 | CCGCGAGCAGCACAGCTCGG | 501 |
| 7600 | GCCGCGAGCAGCACAGCTCG | 500 |
| 7601 | GGCCGCGAGCAGCACAGCTC | 499 |
| 7602 | CGCTTATGGGGAGGGTGGGGAGGG | 634 |
| 7603 | CGGTGGGCGGAGATTAGCGAGAGA | 559 |
| 7604 | CCGGTGGGCGGAGATTAGCG | 558 |
| 7605 | GCCGGTGGGCGGAGATTAGC | 557 |
| 7606 | GGCGCTTATGGGGAGGGTGGGGAGGG | 632 |
| 13684 | CCTGGCACGTGTCCCTGGTCAAG | 3479 |
| 13685 | CTGGCACGTGTCCCTGGTCA | 3480 |
| 13686 | TGGCACGTGTCCCTGGTCAA | 3481 |
| 13687 | GGCACGTGTCCCTGGTCAAG | 3482 |
| 13688 | GCACGTGTCCCTGGTCAAGT | 3483 |
| 13689 | CACGTGTCCCTGGTCAAGTA | 3484 |
| 13690 | ACGTGTCCCTGGTCAAGTAG | 3485 |
| 13691 | CGTGTCCCTGGTCAAGTAGG | 3486 |
| 13692 | ACCTGGCACGTGTCCCTGGT | 3478 |
| 13693 | TACCTGGCACGTGTCCCTGG | 3477 |
| 13694 | TTACCTGGCACGTGTCCCTG | 3476 |
| 13695 | TTTACCTGGCACGTGTCCCT | 3475 |
| 13696 | ATTTACCTGGCACGTGTCCC | 3474 |
| 13697 | AATTTACCTGGCACGTGTCC | 3473 |
| 13698 | AAATTTACCTGGCACGTGTC | 3472 |
| 13699 | GAAATTTACCTGGCACGTGT | 3471 |
| 13700 | GGAAATTTACCTGGCACGTG | 3470 |
| 13701 | AGGAAATTTACCTGGCACGT | 3469 |
| 13702 | AAGGAAATTTACCTGGCACG | 3468 |
| 13703 | CACGTGCGGCCTGTCAAGAGATGA | 5928 |
| 13704 | ACGTGCGGCCTGTCAAGAGA | 5929 |
| 13705 | CGTGCGGCCTGTCAAGAGAT | 5930 |
| 13706 | GTGCGGCCTGTCAAGAGATG | 5931 |
| 13707 | TGCGGCCTGTCAAGAGATGA | 5932 |
| 13708 | GCGGCCTGTCAAGAGATGAG | 5933 |
| 13709 | CGGCCTGTCAAGAGATGAGG | 5934 |
| 13710 | TCACGTGCGGCCTGTCAAGA | 5927 |
| 13711 | GTCACGTGCGGCCTGTCAAG | 5926 |
| 13712 | AGTCACGTGCGGCCTGTCAA | 5925 |
| 13713 | AAGTCACGTGCGGCCTGTCA | 5924 |
| 13714 | CAAGTCACGTGCGGCCTGTC | 5923 |
| 13715 | TCAAGTCACGTGCGGCCTGT | 5922 |
| 13716 | TTCAAGTCACGTGCGGCCTG | 5921 |
| 13717 | CTTCAAGTCACGTGCGGCCT | 5920 |
| 13718 | CCTTCAAGTCACGTGCGGCC | 5919 |
| 13719 | TCCTTCAAGTCACGTGCGGC | 5918 |
| 13720 | TTCCTTCAAGTCACGTGCGG | 5917 |
| 13721 | ATTCCTTCAAGTCACGTGCG | 5916 |
| 13722 | AATTCCTTCAAGTCACGTGC | 5915 |

Hot Zones (Relative upstream location to gene start site)
1-1880
2150-2240
2420-3050
3230-4130
4310-4400
5900-6000
335000-336000

### Examples

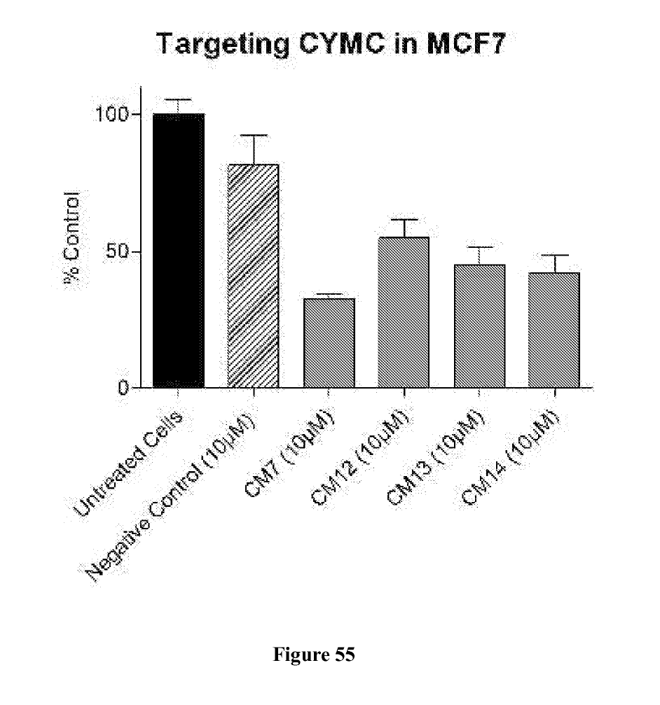

In FIG. 55, CM7 at 10 μM showed statistically significant inhibition compared to control values in MCF-7 (human breast cancer cell line). CM7 (structure shown below) fits the independent and dependent DNAi motif claims

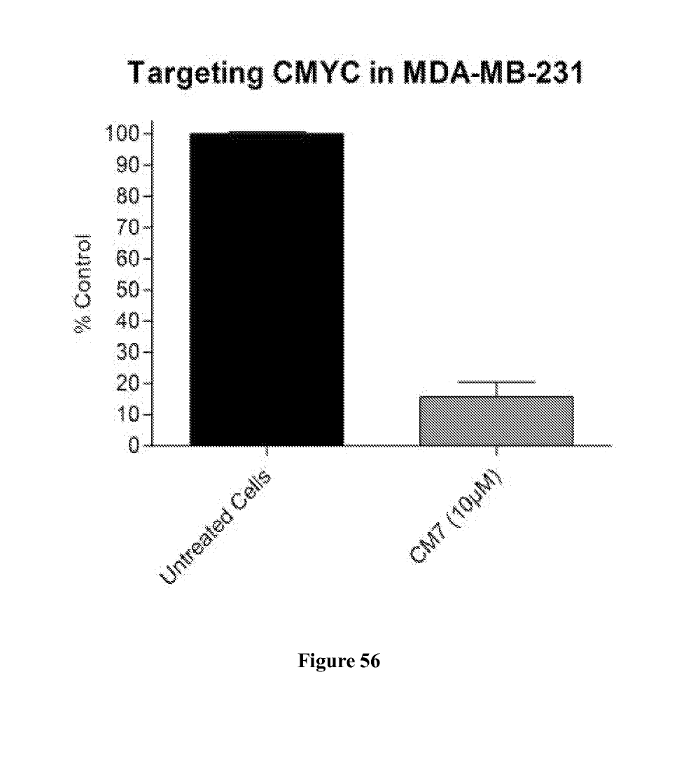

In FIG. 56, CM7 at 10 μM showed statistically significant inhibition compared to control values in MDA-MB-231 (human breast cancer cell line). CM7 (structure shown below) fits the independent and dependent DNAi motif claims.

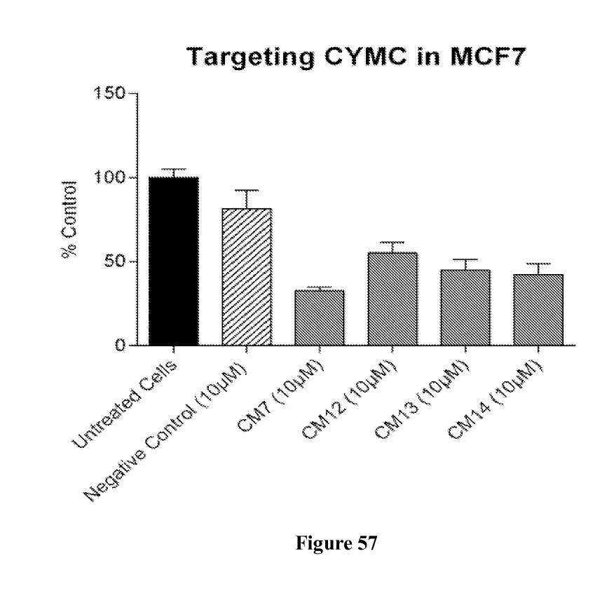

In FIG. 57, In MCF7 (human mammary breast cell line), CM7, CM12, CM13, and CM14 produced statistically significant (P<0.05) inhibition at 10 μM compared to the untreated control values. The CMYC sequences CM7, CM12, CM13, and CM14 fit the independent and dependent DNAi motif claims.

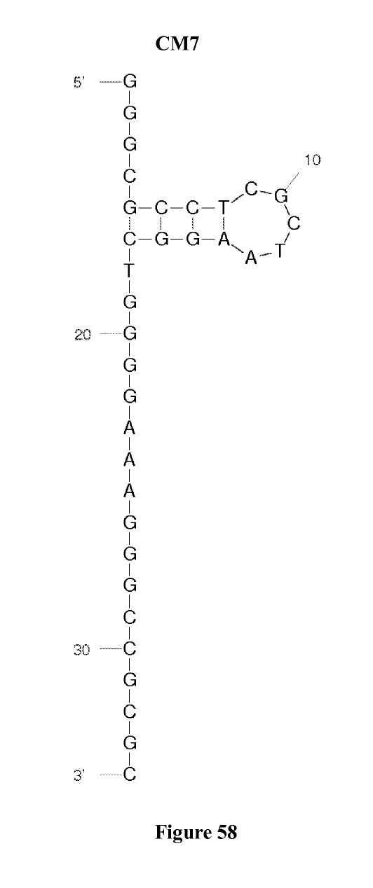

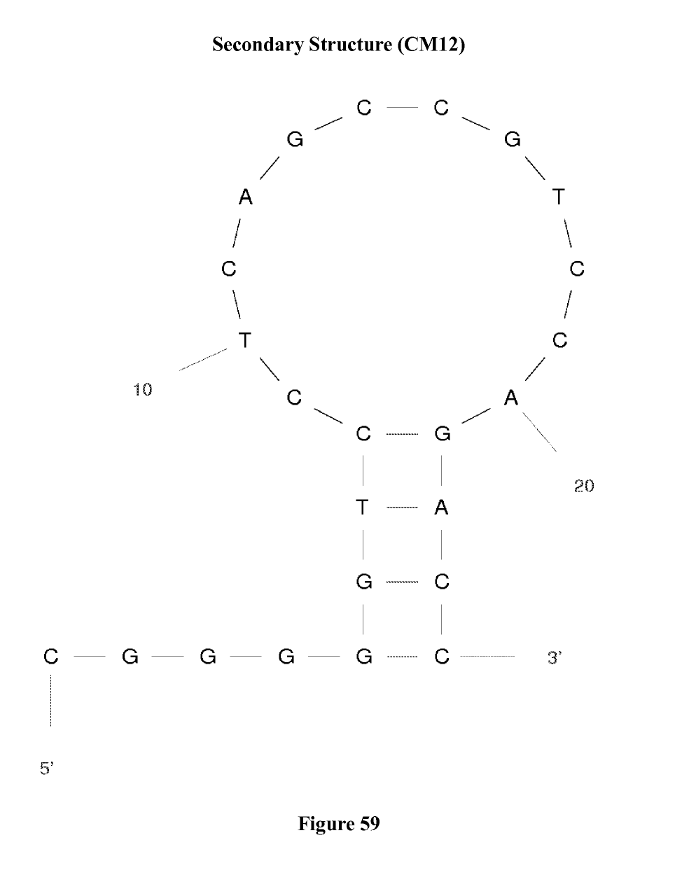

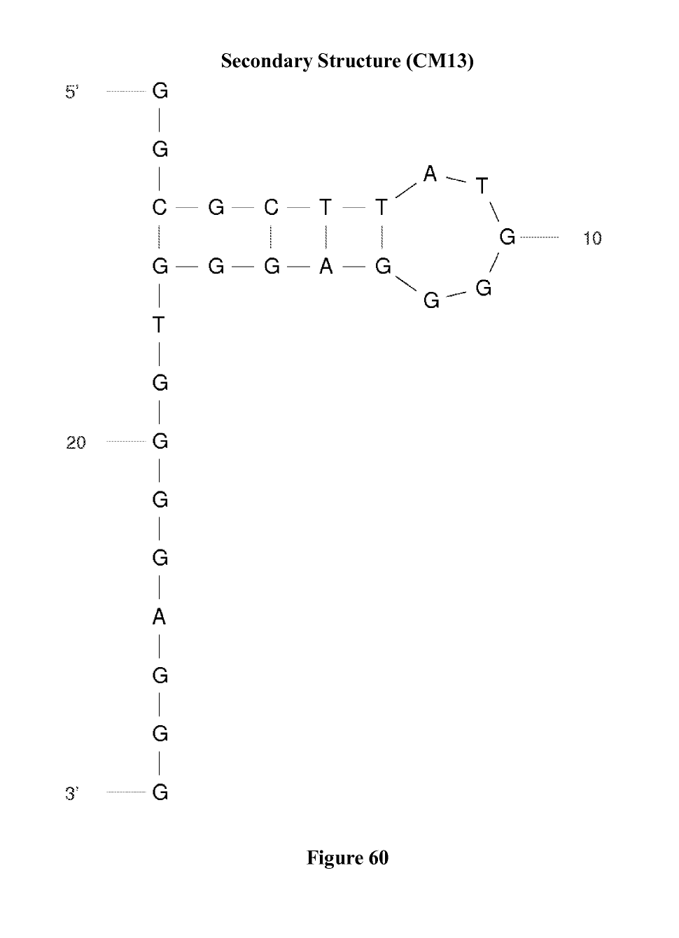

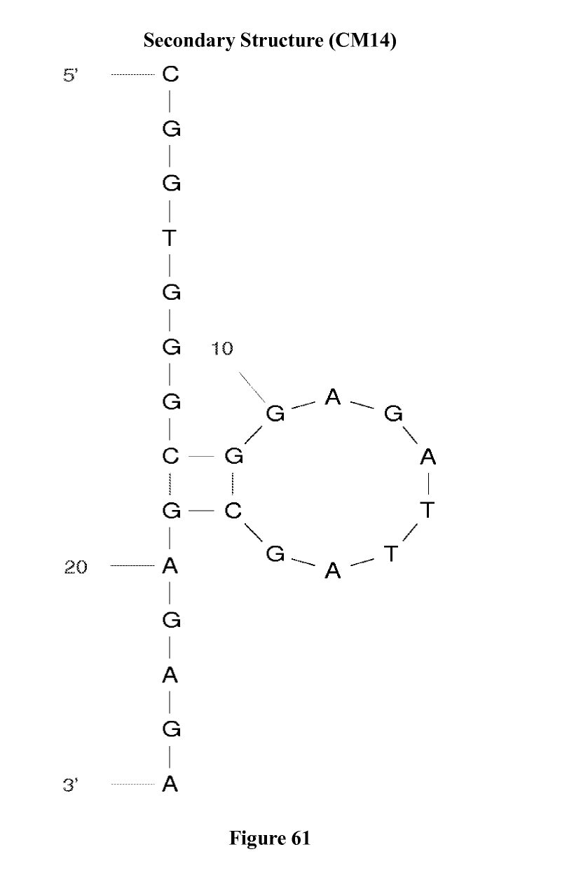

The secondary structure for CM7 is shown in FIG. 58. Sequence 317 (CM7) is shown in FIG. 58. The secondary structures for CM12, CM13, and CM14 are shown in FIG. 59, FIG. 60, FIG. 61.

Genetic Code (5′ Upstream Region)

(SEQ ID NO: 13723)

TGATTGTGGCCAGGCACTACAGCTCACACCTACAATCCCAGCTACTCTGG
AGGCTGAGGTGCGAGGATGGCTTGAGCCCAGGAGTTCAAGACCAGCCTAG
GCAACATAGTGAGACCCTGTCTCTAAAAGGTTTTCTAAAATTAGCCAGGT
GCATATGCCTGCAGTTCCAGATTCTCAGAAGCCAGAAGTGGGGAGGATCT
CTGGAGTTCAGGAGTTTGGGACCACGGTAAGCTATGATTGTGTTACTGCA
CACCAGTTTGGGTGACAGAGCGAGACCCCTTCTCTCAAAACAAATAAATA
AGATTGTGGTGATAGCTATACAACCCTGTGAATAACTAAAAATTGTTGAA
CCATGCACTTTAAGTGCATGAATTTTATGGCATGTGAACTTTATCTCAAT
AAGGCTGCCATTACAAAGCTAAAAAGGGAGGCAGGTGCATGAGCACATAT
ATGTCTAATATTAGCTAAAATAGTATCACCATTATTAAATAAACTTTTAA
AAAATACCTTCTTTCTGAGAATGCAATTCTTCCTTATAATCAGAACCATG
AATATACCAGGAAACTTTTTAAATCAGGGAACAAATGCCTACGAAGGACA
GGCACAAGCCAGAAAGGGACTATGGATGAATTAAGTGGGCTGAGCATATG
GGAGCGGTGGAGACTGGGGCAAACTGAACAGCTCCTGGCCCTTTTAAAAG
AAATCGGCTGCTCCTCAACTTCCATCCACTTCTGAATGCAGTTCCAGAAT
TACCAAATCTGCCTGTTTAAGGAAAGGCACAAATTCAGATTGTTAATGTG
AAATCTATTGACTTGTAAGTGTTGGCACCTATTTTTAAATGTTATAAATG
CTGAGAGGGTCAAAACCTGTCATCCAAGCCAACCTACCAGTAAGCAAGAC
TTGGTCCTCAAGCAAGTTTGCTGCCTCTGCTTTGAGTACTTTAGCATGAC
TTTCAAAACCTCCCACCTCCCCCTCGCCCTGCCTAAACCCACTTTACCCC
TCACCACCACTGCAAGAAGTTATCCAAGCTATGAAGAGAGACAGAAGAAT
TCATACATAAATAAAGAGTCCCAAAACATTCTCAAAGATGCCAAAGTCAG
GCTAGGGTGGCATGGAGAGGGAGTGGGGCATAAAGTTTTTGATTCCTAAT
CTAATTAGAGAGCCCTATAACAGATTCTTTGTTCAAAGACCAAATTTAAT
TTACAATTTTATATCTCCAGTGAAGTCAGCTTTTATTAATTTCCAGCACA
ATATTTGGATATACTGGCCAGAACTTCAATGAGTTCCTATTTAGTGTTTA
ATCTTCTAATGCATTCCAATTAATTATTTCAGTTTTATGGCAAACTGTCT
TCAGCCAACATCCAAGCTGGACACCCCATGCCTCTCCACTCACCCAAAAA
ACCAGCTCGGGAGGTGTCAATATAATGACTTAAGATGCTGAATGGTAAAG
GACAGGATTGGAAGGAAATTGCGCCTGCAATTATGCACTAATGCTTCACC
AGAGAAGCAGATGGCATTCCTTGCATAAATTATTATTTATCCTTGGAATT
CCCCTCTGCCTATTACCAAATCAACCCTTGAAAACAAGTCTTTGTTGGGT
CTGTGAAGTCCCCTGGCCAGTTTCCAATGTCTGCTCCCTCCCTCACATCC
CACCCTCCAGAGCTGCAGCGAGGGTAAGAACTCCAACATGGCCCACAGGC
AAGGGTTTCCGAAAGCATCGACGTTCTAAATACATTTGGACGGAGGTGCA
CAGAAAGGAGTCCGCTTTATTTTGCAGACTGGGAATCCAGATGCAATGAC
CACAGGCAGAAAGCATGGAGCAGAACCTCCCAGCCTCGGCTGTACCCCCA
GTGATAAGGCTTGCCACGTGTGGACGTCACCAGGTTGCCCACCACAGCAC
GGGGCTTAGGCTGTACTGTGCATTCTCTCATGGAATCCTTGAACAAGGAT
TGAGGTGGGCAATGATGTTCCACTTTGAGGAAATGAAATGAAGAAACCAG
AGACTCTGAGACAAAGAAAAGGGCTTTGGGTTTTTTTGTGTTTTTTGGCT
TTTTATTTATTTATTTATTTTGTACAGATGAGGTCTCACTTTGTTGCCCA
GATTGGTCTCAAAGAATGGTGCTTTGGATTAGATCTTATTGTGATGAAAA
ATAAAAAAAAATTAAAAATTTTTTAATTTAAAAAGAATACTGCTTTTTTT
TTTTTTTTTTAACAGGGTCTCTCTCTATAGCCCCTCTCTATAGAGTGTAC
AGTGGCACAATCTCAGCCCGCTGCAACCTCTGCCTCCCAGGTTCAAGCGA
TCTTCCCACCTCAGCCTCCTGAATAGTTAGGACTACAGGCATGTGCCACC
ACGCCTGGTTAATCTTTTGTAGAGATGGGGTTTCGCCATGTTGCCCAGGC
TGGTCTTGAACTCCTGAGCTCAAGCGATCTGGCCACCTCAGCCTCCCAGA
GTGCTGGGATTACAGGTGTGAACCACCATGCCCAGCCAGAACACTGTTAA
CCTTAACATCAACAGGCAGCTACCATTTTCGAGTGCCTGCAATGTCATTT
AACCTTTAGGAACAGCTCTGGGAGACAGCTATAGTTGTTGCCATTTTCTG
CAGATTGAGAAACTGAGGCTCAGTTAAGTGACGTAATTCTAAGGCACCAC
ACCCAGTCAAGCGCAGTGACAGAATTCGAACTCTGGCTTGTAGGGATTCA
CAGGACTGCCAAAGCTTACGCTAACCCATTTCTTCTCCTGTGCACCATCA
TTGCCTCATTCTCTGCCCTCATTTTCTTTATTTATTTTTATTTATTTATT
TTTCTTTTTTTGAGATGGAGCTTCACTCTTGTTGCCCAGGCTGGAGTGCA
ATGGCACGATCTCGGCTCACTGCAACCTCCACCTCCCGCGTTCAAGAGAT
TCTCCTGCCTCAGCCTCCTGAGTAGCTGGGATTACAGGCATGCACCACCA
CGCCCAGCTAATTTTGTATTTTTAGTAGAGACGGGGTTTCTCCATGTTGG
TCAGGCTGGTCTCGAATTCCCTACCTCAGGTGATCCACCCGCCTCGGCCT
CCCAAAGTGCTGTGATTGCAGGCGTGAGCCACCGTGCCCAGCCGCTCTGC
CCTCATTTTCTCCCCAAAACCAAAGTCTACTTTACAAGCACAGATATTAC
TAACTTGTCTTACGAAACTTTCCAGAAGAAAGAGAAAGAATATATGTTTT
ACCAAGCCCCTTGGAGGACAAGGATTTGTTTCTGTATCCACTGTCTCGAT
ACTCATGGTGCCTTTTACCCCTTGGCATTATGCCCCAGGAAAGTGGCAAA
AGTAAGAGGTAACCTCTCCTTCCTTCCTTATTTCCCTAAGGAAATTTGCT
CTGGTCACCAGCAGCAGAGAAATAGAAAGCGCCGGGCACCTGGCTCGACT
GGGGCAGTGACAGGGCAGAGGCGGCCCAGGTTATGGTATCAAAAGGTTTC
TGGTGCTGAATCTCATGACTACTATTCACCGTGTGAGTTTAAGCAAGTCC
CTGCAACACCTCAATTTTCCCCATCTGTTAAATGGAATTTTAACCTACAC
CTCCTAGGATTACTATGGAGATTTAAGGAGGCAATGCAGTGGGGCTTTCT
AACCTTTTTAACTCACTGAGATACATTTCCCGTATCGTCCAAGTGCAGAC
ACACACACACACACACACACAGAGAGAGAGAGAGAGAGAAAAGAATACTT
CATCTGCAACACACTTTGATATTTTCTGTGCCAGCCCATTTTGTGAAATT
GCTCATCATGATTCATTAAATTCATTTCTTATTTACTATTTTTAAATTTT
TATACATGCAGGGGGCTCAAGTGAGGATTTCTTTCATGTATACATTGCAT
AGTCGTCAAGTCTGGTCATTAAATGTATTCATTATCCAAATAGTGAACAT
TGTTAAATTGATTTCATGATCCACTAAGGGGTCATCATTTGCCATTTTAA
AACTCTGACAGTATGAGCTTCTCCCTAGCCCAGTTCCTGTTACCATCTTC
CCATTCTTCCCTTCCTTCTTCAATTCAGATAGGATTTTCCTCCAGAGGGA
TTATAAAGTTGCGAGGAAAGCGCCTGCAGGGGGTGCTGTTCCACACTGTT
GTTGAAGTGTGGTTTGGTTTTTATTTCGTTGCATTTGCTTTTCGGTCAAT
GAGGGCAATTCATCTGGAATGACCCCCATCCTCGTCACCCTTGCTCCAAC
GATGTTGGGGCCCAGCTCATCAACAAGGACACCTGAACAGAGCCCTACCC
ATTGATGGAACCGAAGCAAGGGCAAGGAAGAGTTCTCAACCCTTCTCTCT
ATATACGATTAAAACTGGGTTAGGCTAGGTGTGCCCTCAGCTCAGAAGCT
CTCTCTAATAGCATTCCTTCACTAAGCACTTACAGAGTGCCTACCACGTG
CCAGGCATTGTGCTGGGCTCTGGAGACCACCTACTCTGTGAATGGCACCT
TGAGGCTTGATGGGTGAGAACGCGAGTAAAACACAATCCATACTGACCCC
AGAAGCTTCTCCTCAAGGAATCAGACATTAAAAAGCACAAAAACTATAAA
GTTGATTTTTTTTTTTTTTTTTTTTTTGAGACAAGAGTCTTGCTCTGTCA
CCCAGGCTGGAGTGCTGTGGCACCATCTGGGCTCACGGCAACCTCCACCA
CCCAGGTTCAAGCAATTCTCCTGCCTCAGCCTCTCGAGTAGCTGGGATCA
CAGGCATGCGCCACCATGCCCCGCTAATTTTTGTCATTTTTAGTAGAGAC
AGGGTTTCACCATCTTGGCCAGACTGGTCTCGAAATTCTGACCTCGGGTG
ATCTGCTCACCTCAGCCTCCCAAAGTGATGGGATTACAGGCATGAGCCGC
TGCATCTCTGGCCAAAACTTGAATGTTTGTTTGTTTTGAGACAGGATCTC
ACTCTGTCATCCAGACTGGAGCACAGTGACACAATCTTGGCTCACTGCAG
CCTCAACAGCCACGGCTCAAGCAATCTTCCTCCTCCACCTCAGTTTTCCA
AGTAGATAGGATTACAGCCATGAGGCACTGCACCCAGCTAATTTTTTTTT
TTTAATTTTTTTGTAGAGACAGGGTCTTACTGTGTTGCTCAGGCTGGTCT
CAAACTCCTGGGCTCAAGTGATCTGCCGGCCTTGGCCTCCCGAAATGCTG
GGATTACAGGTACGAGCCACCACGCCTGGCCAAACTTGTATTTTCTAAGA
CAGAAGAATGAGGGGATGGTTTAAACTCTCAAGGGAAGGGGAAAGGATCA
TGAAAAGCTCCTACAGGAAGATGCTTGAGTTGGATTACTAAGACATATGA
GCAGAGATGGCAGGCTGGCAGCCTGAGGGCCACCTCTGCCCATAGACATG
CTTTGCTTCTCCATATCATTTTTTTTCCCAACACACTGCTGCTGGCTTGA
AATCTCCATATAATTCTTACAATAAGTTGTTAACATTTTAAAACCTGGAT
TTCCACCTTCCCTGAAAAACTGGAAGCATTTCCACCCATGGGCCCATATT
TCAGGGTAACCACCAGAGCAGGTGCCAAATGGGAGCCACCAGACCTACAC
AGGCAAATGCTCTCCAGTTTACCAGTCTCCACCACTCCCTATTGTATTCT
TCGTTTACATTTCCTGCCAAACCTCTGTAAGCATCTGAGTTGGCAACCCT
TGATGTGTTAGCGGAAAATGTGGATCAGAAGTTAGAAAGAGTTTCTAAAC
CTGGTTGTTGATTTACGCTTTATGCTTTGAAGGAAAACAGTTTTTCCAAT
GCCCAGATCCACTCACCAAGACAAAAAAAAAAGCAAGCTGTAGATTTCAG
TAGCAGCCTTGTCTAGCCAGCAATAAAGGTGCCCTGGGTTTCCAGGACCA
CACCCCAGGGATTAGCCCCGGGGCATCATATGAATTCAGTGAAAGGCGGG
AAATCCTAACATAAAGCGTTGATTCGTATTAAATAGGAACAATGCCTAAT
TCTGCCTTCCTGAACTTCCAGAATTTTGCTTTTTCTGAATAGAGTGATCT
GCAAAACAGCATACACTTGGAATAGTAAGTCGTGCAAGAGTTGGAGACAG
GAAGGGGGTGGGTTTGGAATTGTCTCCAAACATTAGATAATCTCTTTGTG
ATTCTAAACCTCAACTTGACAAGCTTGTATTAGTCCACAATTTTTCACAC
TTGATGAAGTGATAAAGGACATCAATTTCATGGAACTCACTATGAAACAC
CATGCAATATTGATACATTTAACTTAAAACAGCTCAATACATAACTTTCT
GCTAAATCTGGAACTCACATTAACAATTGCTAACATTTGCTGAGTGTGGG
CCAGACAGCAGGCTCTGTGCTGAATGCCTTATCTCACTTAATTCCTGTAA
CACCTTCAATAAGATAGGTGCTACAATTATAGTAATCCCATTTTACAGAT
GAGAAAAGTGAGATTCAGAGAGGTCATGTGACTTGACAGATTTATCAGGT
GATCATGACAGAGTAGTCCTCCAACCAAGCTGATTCAGCAACCCGTCCTT
ATATTCTAGATTCTTGTGTAGCCAAAAAGTTATTGAGAAAGTCTGCCCAT
TGACTTCATTCTCTTACCCAGTGTAGAGTCAGCATACATTCATTCACATT
AACTATGGGCCAGACTTGATTCCTGGCCTTGGGACTTTTTTTTTTTTTTG
GCAGGGGCTGATAACATTCTATTTTATTTATTTATTTATTTCTCTCTCTT
TTCTTTTAATTATACTTTAAGTTCTGGGATACATGTGCAGAACATGCAGG
TTTGTTACATAGGTATACACATGCCATGGTGGTTTGCTGCACCTACCAAC
CCATCATCTACATTAGATATCTCTTCTAATGCTACCCCTCCTCTAGACCC
CCGGGACATTTATAATCTCATGAAGAAGAGAAAAAGGAGGCCTTTCTCTG
ACAGCTAGAAAACCACAGTTAGTCTATTTTAGCCGGAGACCCTGGATTCT
ACCCTGAGAACAAAGGTTTATGTTTCAGCAGCTTAATTAGAGGTTTTCCA
GAACTTTTTCTGGCTCCATGCTTTTATGATTCTGTAAGATGATCATGGGA
AAAGGAAGAGTCCACAGAGAAAATGGGGCTTGAACTTGGGCTGGGAGGAA
AGGTGGTTCTTAGATAAATCAAGAAGAGAAGAGACAGTAAGTCTGGGGAA
CTGCCTGAACCAAAGTGCTGAGGTGGAAACTTGTGTGTCACTCAGAGTGG
CTGTAAATAGACCTGTTTCTCTGAAGTGCAGAGTTGGTAAGAAATAGGGT
AAGATAAGGAGGAGGCCAGATGCATGAGGGCTTGGAATTCCAAGCTCATA
ATGGAGAACCTCATTTTGGACCATGGGGGTCAACTGAAGAATTTTAAATG
AAGAGGAAAATTAATCAGTGTGCAAGGTTAAATGGAGTGGCAGAGACTAG
GAGCTATTAGGAATCTACTGCAAGATGATTCTAACAGCCATAGGTAGTGG
GTAAAAGAGGAAAGTGAGCCAATAAGGGAAACAGAAGAACAAGTTGAATA
TGTGGGAATATAATCAGGAGAATGTGGAGTGAACCCAGGGATTCTCAACC
TCAGCACTGGTGACATTTTGAGCCCCACTCTCTGTTGTGGGAGCCGTCCT
GTGCAATGTAGGACATTTAGCAGCATCGTTGGCCTCTACCCACTAAAAAC
GCCAAGTAGTAAGGCCCCATCCCTTAGTGACAACCCAAAATGTCTCCAGA
CATTGCCAATTGCCTCTGGTTGAGACCCACTGATTTATGGAAATCAAAAG
AATAATCATTTCCAAAAGCCTGACAGCAAACAGCAAAGTCCAAAAATAAA
ATGAGAACAAGACCATTGGCTTTGGTGGTTGGAGGTCATGAGATACCTTC
AAGAAAGCACCCTCTATAATATCAAGTCCATGTAGAAGACGTTACAGAGG
AAACAAACAATATAAAAGTGAAAACAACCAAGGGTGAGCTACTCTCAGAA
AGTATGCCTTTGAAAAGAAAGTCAGAAGCCATAGCTTGGGATCTCTGCAG
ATCCCTAAAAGAATAGATTCCTATTCTACTGACTTTCTATGAAGATCAAA
TTGTAAAAAGCAAAAGTTTCTCTCCAAGGGTTTCCTTTGCAGTGACCTGT
ATGTCCAACCACGCAAGGGCCCATTGTGGGGACATATGTTGTCCAAAAGG
ACCATAGCAGAGACAGGCCAGTGAGCCAAAGTGTGGAAACTTTTGAGACT
GGCTTGAGCTTGGCACTTATAGAACAATAAACCAAGCCTTTGAAGGGGTT
CAACAAAGGAACCATTTGTCCACTCTAGTAGCTACAAAGTAAGGCAGGGT
TGCAGCAAAGAACAAAAAAATAAAAGAAGGCCAAGCTGGAGGTATGACCA
AAGTTTACTAGGTCCATTCTGAGACCTTCTGCTAGGGTCTGAGATCTAGA
AGACAGTGAATAAGGAAACAAACCCAAAACTCAACGCAACACAGGATATG
GAAGCTCTCAGGCCTGACGTTAACAGCATCTACTATTTTTCTTCTCAGCT
ACTTTAATGAATGCAGTATACTAAAAGCCAGGAGGGGAAGGGACAACACT
AAGCAAAAAACATGCATTTTTTAAAATGCACAGATTTTCTTCACTGCCGT
TTTTGTTATCATTCCTATGAATTAGTGATGCCGAATTTCATTTTCTCATC
TGCTGAAGAGCTTTCCTGTGTTCCTCTCGTTGGAACACATGCTTGGCATT
AAAATGCTTGTGAGAACTTCTCTTCCTTTAACGTTCCCTGGCTAGCTTGG
TTTTTAATCTAACAGCCCTTCTTTCAAAATGATCCTTCCACTGGAGATAG
ATATTTATCATTCTCTTCCTTCACCTCATCTCTTGACAGGCCGCACGTGA
CTTGAAGGAATTTTTCAAATAGCAGCTCAGCCACCCTGAGGGGCTTCAGT
CTCACCCCTAAGTTCGCTGGCTTTTTCTTCACCACGTCCAGTTGCTTTCC
ATCTTATTAACTGCTCTTTTCACTAGAGGACCAACTCAGTAGGAAATTTT
TTGAGAGGTGGAGAAAGAGATGTTCAAAGAAGGTGTTGGGGTCGGGGGAA
ACTGGTTTTATTTTATACAAGTCACACATTCTGAATCTTCCCTTTTGTGT
CTCTGGGGAGAAAGGAGAAAGTTTGATCAAATCGCTCATTATTTCTGCAC
TTCTTTCTTTTTTCCTAAGTATAAAAATATATGACTACTACTACTGTGAG
ACTATGTGATTGTGAGAATGAATGATTCTTTTTTTTTTTTTTTTTTTTTT
GAAACGGAGTCTCTCGCTGTCACCCAGGCTGGAGTGCAGTAGCACGATCT
TGGCTCACTGCAACATCTGCCTCCCGGGTTCAAGCAATTCTCCTGCCTCA
GCCTCCTGAGTAGCTGGGACTACAGGTGCGCTCCACCACCCCCAGCTAAT
TTTTGTATTTTTAGTGGAGACGGGGTTTCACCATGTTGGTCAGGCTGGTC
TTGAACTCCTGACCTCATGATCCTCTCACCTCGGCCTCACAAAGTGCTAG
GATTACAGGCGCATGGCCAAGAATGAATGATTATTTGTGCCTTCCTATGT
GAAAAAAAAATGTTTCCTCTAGCTACACACTATTCTGTTCTGTGAGGCCG
CCCCATCAGACTGTTGACCTAGAGTCCCAACCCCGGCCCTCCAGGAGACC
TGCCTGTTCTTAGAAGCCCAACCCACTCAGCAGCAGCTCCAAATAACAGG
GGGAGCCAACAAAAAAGAGTGCTGCTAGAGCAACAAGCAAGGGGCAATTA
GTCAGAAGGCAACTTCCATGGTCTTCCAAAAAAAATTGAGGTGAAAGACC
AAAGATGTCCCTAAAATGTCTTCCTAAAAGATAAACTTCATCAACTACCT
CTGACTGGTCAGTATTAAGAACCACTTTCAGGCCAGGTGTCATGGTTCAC
GCCTGTAACTCCATCTACTCCAGAGGCTGAGGCAGGACAATTGCTTCAGG
CCGGAGGATTGCTTGAGGCCAGGAGCTGGAGACCAAGCCTGAGCAACACA
GTGAGACCTCATCTCTACCAAAAATGTACCTCTATTAAAAAACAAAAAAG
AAGAAGAAGAAGAAGAAGAAGGAGAGGAGGCTGGGTATGGTGGCTAATGC
CTTTGTAATCCCAGAACTTTGGAAGGCTGAGGCAGGAGAATCACTTAGGC
TGAGGCAGGAGAATCACCAGAGTCTAGGAGTTTGAGACCAGCCTGGGCAA
CATAGTGAGACCCCCATCTCTACAAAAAAAAAAATTCAAAAATTAGCCAA
GCGTGGGGTTTGTGCCTGTAGACCCAACTACTCAGGAGGCTCAGGTAGGA
GGATCACCTGAGTCCAGGGAGGTCGAGGCTGCAGTGAGTCATGATTATTC
CACTGCCTTCCAGCCTAGACTACAGGGTGAGACCCTGTCTTAAAAAAAAA
ATTAAAGAAGAAAAAACTCTCTTTTCTTTTCTTTCTTTCTTCTTCTTCTT
TTCTTTTTTTTTTTTTCTTTTTTTTTTTTTAGAGATGGCACGTCACCACA
TTGCCCAGGCTGTTGTCGAACTCCTGGCCTCAAACGATGCTCCCACTTGA
GCCTCCCAAAGTGCTGGGACTACAAGCATAAGCCACCACACACGGCCTTT
TCCTTTCTTTTTCTATTTCTCAATGGATTTTTCCAATGGACACGTATCAC
TTTGGTAGTTATACATGATACTAGTTGTAATCTCAGCCATTTTTCAACCC
AGCAAATGTCTATTCTAGGTCAAATATGTCTCAAAAATTACTAAAAGAAA
ATCAGTTATGTCCTTTAACCTGGCTGAGGTCTGGCTTTGTTTTCTCTCAT
GTAAAAATGGAGATGGCACAAAACAACTCCAAGCTGTTACTTGAAAGTAA
CACCTCAGGTGATGTCACCAGCCTGAGGGAGAGTGAGGTTAAGTTCTGAA
CCCACAGGCATTATATCTGCCTGGGGTTCACATGCCCTACACTGGACTGG
CATAATTTGAGAGTCAGATCCGAAGATGTGGTATATCCGCCATCTTTAGC
AACTTTCAAAAACTACCCTATGAGGTCAAGCTGGACCTACTTTTGGTTTT
GCCATTGTTGTTTGTTTGTTGTTGAGGGTTTTCTTTGAGGGGCGGGGAGT
GCATGCCCCTGTGGAGAGCACTCATTTAGCTTCAATTAGAGTAATGCCAA
AAGTGCCAGATTCCTGGGAAATCAGCCTACAAGGCTCCTGCGGGAAGGAA
CCTCCACTGCCAGAAGTCCTTAGGGCATCTAAGTGATCAGACACCGTCAG
GGATTCTTTGCCCCGTAAAAACCTACTTGACCAGGGACACGTGCCAGGTA
AATTTCCTTCACATTTACTTCAACCTTATTGCATACTCATTTTAGTATTA
AAACCTTTAATAAAATGCTCCTATTCCTTCACACTTTTTTTCTATGAGAT
CTCAAATACCCCTTCTTGCTATTAAAAAAAATCACTTATTATTCACCAGC
CCAATATTTTAAAAGTAAAAATAATAAGCCAAGGCCAGGAGCGATGACTC
GCACTTGTATTCCCAGCAGTTTCAGAGGCAAAGGCCGAAGGATCGCTTTA
ACCGAGGAGTTTGAGACCAGCCTGGGCAACATGACCAGACTGCCTCTCTA
CAAAAAGTTTAAAAAATTAACCGGGTGTGGTGGTGCACTGCACTCCCAGC
TACTGGGCTGGGGTATCAGGCTGAGGTAGGAGGTTTGCTTTGAGCCCGGG
GGGATCGAGGCTGCAGTGAGCTTTGATTGTGCCACTGCACTCCAGCCTGG
GTGACAGAAGGAGACCCTGTCTCAAAAATAATAAGAATAATAATTAATAA
TAATAGGCCAAACCAAATACCCATCACCTTCTGCTGTGCCTCCCCTTTCC
CCAATAAATCCAGTGTCTTGCTTTCAAATTTTGTGGTTAAAAAAGATGAT
GAGTTTCTAAGACGTGGGGGCTAAAGCTTGTTTGGCCGTTTTAGGGTTTG
TTGGAATTTTTTTTTCGTCTATGTACTTGTGAATTATTTCACGTTTGCCA
TTACCGGTTCTCCATAGGGTGATGTTCATTAGCAGTGGTGATAGGTTAAT
TTTCACCATCTCTTATGCGGTTGAATAGTCACCTCTGAACCACTTTTTCC
TCCAGTAACTCCTCTTTCTTCGGACCTTCTGCAGCCAACCTGAAAGAATA
ACAAGGAGGTGGCTGGAAACTTGTTTTAAGGAACCGCCTGTCCTTCCCCC
GCTGGAAACCTTGCACCTCGGACGCTCCTGCTCCTGCCCCCACCTGACCC
CCGCCCTCGTTGACATCCAGGCGCGATGATCTCTGCTGCCAGTAGAGGGC
ACACTTACTTTACTTTCGCAAACCTGAACGCGGGTGCTGCCCAGAGAGGG
GGCGGAGGGAAAGACGCTTTGCAGCAAAATCCAGCATAGCGATTGGTTGC
TCCCCGCGTTTGCGGCAAAGGCCTGGAGGCAGGAGTAATTTGCAATCCTT
AAAGCTGAATTGTGCAGTGCATCGGATTTGGAAGCTACTATATTCACTTA
ACACTTGAACGCTGAGCTGCAAACTCAACGGGTAATAACCCATCTTGAAC
AGCGTACATGCTATACACGCACCCCTTTCCCCCGAATTGTTTTCTCTTTT
GGAGGTGGTGGAGGGAGAGAAAAGTTTACTTAAAATGCCTTTGGGTGAGG
GACCAAGGATGAGAAGAATGTTTTTTGTTTTTCATGCCGTGGAATAACAC
AAAATAAAAAATCCCGAGGGAATATACATTATATATTAAATATAGATCAT
TTCAGGGAGCAAACAAATCATGTGTGGGGCTGGGCAACTAGCTAAGTCGA
AGCGTAAATAAAATGTGAATACACGTTTGCGGGTTACATACAGTGCACTT
TCACTAGTATTCAGAAAAAATTGTGAGTCAGTGAACTAGGAAATTAATGC
CTGGAAGGCAGCCAAATTTTAATTAGCTCAAGACTCCCCCCCCCCCAAAA
AAAGGCACGGAAGTAATACTCCTCTCCTCTTCTTTGATCAGAATCGATGC
ATTTTTTGTGCATGACCGCATTTCCAATAATAAAAGGGGAAAGAGGACCT
GGAAAGGAATTAAACGTCCGGTTTGTCCGGGGAGGAAAGAGTTAACGGTT
TTTTTCACAAGGGTCTCTGCTGACTCCCCCGGCTCGGTCCACAAGCTCTC
CACTTGCCCCTTTTAGGAAGTCCGGTCCCGCGGTTCGGGTACCCCCTGCC
CCTCCCATATTCTCCCGTCTAGCACCTTTGATTTCTCCCAAACCCGGCAG
CCCGAGACTGTTGCAAACCGGCGCCACAGGGCGCAAAGGGGATTTGTCTC
TTCTGAAACCTGGCTGAGAAATTGGGAACTCCGTGTGGGAGGCGTGGGGG
TGGGACGGTGGGGTACAGACTGGCAGAGAGCAGGCAACCTCCCTCTCGCC
CTAGCCCAGCTCTGGAACAGGCAGACACATCTCAGGGCTAAACAGACGCC
TCCCGCACGGGGCCCCACGGAAGCCTGAGCAGGCGGGGCAGGAGGGGCGG
TATCTGCTGCTTTGGCAGCAAATTGGGGGACTCAGTCTGGGTGGAAGGTA
TCCAATCCAGATAGCTGTGCATACATAATGCATAATACATGACTCCCCCC
AACAAATGCAATGGGAGTTTATTCATAACGCGCTCTCCAAGTATACGTGG
CAATGCGTTGCTGGGTTATTTTAATCATTCTAGGCATCGTTTTCCTCCTT
ATGCCTCTATCATTCCTCCCTATCTACACTAACATCCCACGCTCTGAACG
CGCGCCCATTAATACCCTTCTTTCCTCCACTCTCCCTGGGACTCTTGATC
AAAGCGCGGCCCTTTCCCCAGCCTTAGCGAGGCGCCCTGCAGCCTGGTAC
GCGCGTGGCGTGGCGGTGGGCGCGCAGTGCGTTCTCGGTGTGGAGGGCAG
CTGTTCCGCCTGCGATGATTTATACTCACAGGACAAGGATGCGGTTTGTC
AAACAGTACTGCTACGGAGGAGCAGCAGAGAAAGGGAGAGGGTTTGAGAG
GGAGCAAAAGAAAATGGTAGGCGCGCGTAGTTAATTCATGCGGCTCTCTT
ACTCTGTTTACATCCTAGAGCTAGAGTGCTCGGCTGCCCGGCTGAGTCTC
CTCCCCACCTTCCCCACCCTCCCCACCCTCCCCATAAGCGCCCCTCCCGG
GTTCCCAAAGCAGAGGGCGTGGGGGAAAAGAAAAAAGATCCTCTCTCGCT
AATCTCCGCCCACCGGCCCTTTATAATGCGAGGGTCTGGACGGCTGAGGA
CCCCCGAGCTGTGCTGCTCGCGGCCGCCACCGCCGGGCCCCGGCCGTCCC
TGGCTCCCCTCCTGCCTCGAGAAGGGCAGGGCTTCTCAGAGGCTTGGCGG
GAAAAAGAACGGAGGGAGGGATCGCGCTGAGTATAAAAGCCGGTTTTCGG
GGCTTTATCTAACTCGCTGTAGTAATTCCAGCGAGAGGCAGAGGGAGCGA
GCGGGCGGCCGGCTAGGGTGGAAGAGCCGGGCGAGCAGAGCTGCGCTGCG
GGCGTCCTGGGAAGGGAGATCCGGAGCGAATAGGGGGCTTCGCCTCTGGC
CCAGCCCTCCCGCTGATCCCCCAGCCAGCGGTCCGCAACCCTTGCCGCAT
CCACGAAACTTTGCCCATAGCAGCGGGCGGGCACTTTGCACTGGAACTTA
CAACACCCGAGCAAGGACGCGACTCTCCCGACGCGGGGAGGCTATTCTGC
CCATTTGGGGACACTTCCCCGCCGCTGCCAGGACCCGCTTCTCTGAAAGG
CTCTCCTTGCAGCTGCTTAGACGCTG

31. APP

Amyloid beta (A4) precursor protein is encoded by the APP gene. The amyloid precursor protein (APP) is found in many tissues and organs, including the brain and spinal cord (central nervous system). Its function is not well understood, however, it is believed to bind other proteins on the surface of cells or help cells attach to one another, thereby directing the migration of nerve cells during early development. APP is cleaved by enzymes to create smaller peptides (soluble amyloid precursor protein (sAPP) and amyloid beta (β) peptide) which may be released outside the cell. sAPP has growth-promoting properties and may play a role in the formation of nerve cells (neurons) in the brain both before and after birth. The sAPP peptide may also control the function of certain other proteins by turning off (inhibiting) their activity. Alzheimer's disease (AD) pathogenesis is widely believed to be driven by the production and deposition of the amyloid-beta peptide (Murphy and Levin (2010) J Alzheimers Dis. 19(1):311-23).

Protein: Beta Amyloid Gene: APP (Homo sapiens, chromosome 21, 27252861-27543446 [NCBI Reference Sequence: NC_{—}000021.8]; start site location: 27542938; strand: negative)

Gene Identification

| | |
|---|---|
| GeneID | 351 |
| HGNC | 620 |
| MIM | 104760 |

Targeted Sequences

| | | | Relative upstream |
|---|---|---|---|
| Sequence | Design | | location to gene start |
| ID No: | ID | Sequence (5′-3′) | site |
| 7607 | | CGCGACCCTGCGCGGGGCACCG | 1 |
| 7741 | | GTGCGAGTGGGATCCGCCGCG | 34 |
| 7875 | | CGCGCCGCCACCGCCGCCGTCTCCCGG | 68 |
| 8009 | | CGCGCACGCTCCTCCGCGTGCTCTCG | 101 |
| 8143 | | CCGAGGAAACTGACGGAGCCCGAGCGCGG | 137 |
| 8145 | | CGAGTCAGCTGATCCGGCCCACCCCG | 186 |
| 8310 | | CGAGAGAGACCCCTAGCGGCGCCG | 221 |
| 8475 | | CGCCCGCTCGCGCCGGGAGGGGCCCTCG | 256 |
| 8640 | | CGCGCCCACAGGTGCACGCGCCCTTGGCG | 289 |
| 8805 | | GGCCGACGGCCCACCTGGGCTTCG | 351 |
| 8825 | | CGCTGAGGCTCTAGAAAAGTCGAGAG | 446 |
| 8843 | | CTCGTCCCCGTGAGCTTGAATCATCCGACCC | 480 |
| 8912 | | AGGCGTTTCTGGAAGAGAATGAGAACG | 604 |
| 8927 | | CGTCAAAAGCAGGCACGAGCAACCTG | 701 |
| 8928 | | GAACGAACCAAAGGAGCAAGGCG | 742 |
| 8929 | | CGCTGACAAGGGTGCCTAGGCCCGG | 1318 |
| 8948 | | CGCAATTCCGTATTTGTTCCGG | 1738 |
| 8969 | | GTACGTTGGCAGACGCAGTGACG | 4923 |

Target Shift Sequences

| | | Relative |
|---|---|---|
| | | upstream |
| | | location to |
| Sequence | | gene start |
| ID No: | Sequence (5′-3′) | site |
| 7607 | CGCGACCCTGCGCGGGGCACCG | 1 |
| 7608 | GCGACCCTGCGCGGGGCACC | 2 |
| 7609 | CGACCCTGCGCGGGGCACCG | 3 |
| 7610 | GACCCTGCGCGGGGCACCGA | 4 |
| 7611 | ACCCTGCGCGGGGCACCGAG | 5 |
| 7612 | CCCTGCGCGGGGCACCGAGT | 6 |
| 7613 | CCTGCGCGGGGCACCGAGTG | 7 |
| 7614 | CTGCGCGGGGCACCGAGTGC | 8 |
| 7615 | TGCGCGGGGCACCGAGTGCG | 9 |
| 7616 | GCGCGGGGCACCGAGTGCGC | 10 |
| 7617 | CGCGGGGCACCGAGTGCGCT | 11 |
| 7618 | GCGGGGCACCGAGTGCGCTG | 12 |
| 7619 | CGGGGCACCGAGTGCGCTGC | 13 |
| 7620 | GGGGCACCGAGTGCGCTGCT | 14 |
| 7621 | GGGCACCGAGTGCGCTGCTG | 15 |
| 7622 | GGCACCGAGTGCGCTGCTGT | 16 |
| 7623 | GCACCGAGTGCGCTGCTGTG | 17 |
| 7624 | CACCGAGTGCGCTGCTGTGC | 18 |
| 7625 | ACCGAGTGCGCTGCTGTGCG | 19 |
| 7626 | CCGAGTGCGCTGCTGTGCGA | 20 |
| 7627 | CGAGTGCGCTGCTGTGCGAG | 21 |
| 7628 | GAGTGCGCTGCTGTGCGAGT | 22 |
| 7629 | AGTGCGCTGCTGTGCGAGTG | 23 |
| 7630 | GTGCGCTGCTGTGCGAGTGG | 24 |
| 7631 | TGCGCTGCTGTGCGAGTGGG | 25 |
| 7632 | GCGCTGCTGTGCGAGTGGGA | 26 |
| 7633 | CGCTGCTGTGCGAGTGGGAT | 27 |
| 7634 | GCTGCTGTGCGAGTGGGATC | 28 |
| 7635 | CTGCTGTGCGAGTGGGATCC | 29 |
| 7636 | TGCTGTGCGAGTGGGATCCG | 30 |
| 7637 | GCTGTGCGAGTGGGATCCGC | 31 |
| 7638 | CTGTGCGAGTGGGATCCGCC | 32 |
| 7639 | TGTGCGAGTGGGATCCGCCG | 33 |
| 7640 | GTGCGAGTGGGATCCGCCGC | 34 |
| 7641 | TGCGAGTGGGATCCGCCGCG | 35 |
| 7642 | GCGAGTGGGATCCGCCGCGT | 36 |
| 7643 | CGAGTGGGATCCGCCGCGTC | 37 |
| 7644 | GAGTGGGATCCGCCGCGTCC | 38 |
| 7645 | AGTGGGATCCGCCGCGTCCT | 39 |
| 7646 | GTGGGATCCGCCGCGTCCTT | 40 |
| 7647 | TGGGATCCGCCGCGTCCTTG | 41 |
| 7648 | GGGATCCGCCGCGTCCTTGC | 42 |
| 7649 | GGATCCGCCGCGTCCTTGCT | 43 |
| 7650 | GATCCGCCGCGTCCTTGCTC | 44 |
| 7651 | ATCCGCCGCGTCCTTGCTCT | 45 |
| 7652 | TCCGCCGCGTCCTTGCTCTG | 46 |
| 7653 | CCGCCGCGTCCTTGCTCTGC | 47 |
| 7654 | CGCCGCGTCCTTGCTCTGCC | 48 |
| 7655 | GCCGCGTCCTTGCTCTGCCC | 49 |
| 7656 | CCGCGTCCTTGCTCTGCCCG | 50 |
| 7657 | CGCGTCCTTGCTCTGCCCGC | 51 |
| 7658 | GCGTCCTTGCTCTGCCCGCG | 52 |
| 7659 | CGTCCTTGCTCTGCCCGCGC | 53 |
| 7660 | GTCCTTGCTCTGCCCGCGCC | 54 |
| 7661 | TCCTTGCTCTGCCCGCGCCG | 55 |
| 7662 | CCTTGCTCTGCCCGCGCCGC | 56 |
| 7663 | CTTGCTCTGCCCGCGCCGCC | 57 |
| 7664 | TTGCTCTGCCCGCGCCGCCA | 58 |
| 7665 | TGCTCTGCCCGCGCCGCCAC | 59 |
| 7666 | GCTCTGCCCGCGCCGCCACC | 60 |
| 7667 | CTCTGCCCGCGCCGCCACCG | 61 |
| 7668 | TCTGCCCGCGCCGCCACCGC | 62 |
| 7669 | CTGCCCGCGCCGCCACCGCC | 63 |
| 7670 | TGCCCGCGCCGCCACCGCCG | 64 |
| 7671 | GCCCGCGCCGCCACCGCCGC | 65 |
| 7672 | CCCGCGCCGCCACCGCCGCC | 66 |
| 7673 | CCGCGCCGCCACCGCCGCCG | 67 |
| 7674 | CGCGCCGCCACCGCCGCCGT | 68 |
| 7675 | GCGCCGCCACCGCCGCCGTC | 69 |
| 7676 | CGCCGCCACCGCCGCCGTCT | 70 |
| 7677 | GCCGCCACCGCCGCCGTCTC | 71 |
| 7678 | CCGCCACCGCCGCCGTCTCC | 72 |
| 7679 | CGCCACCGCCGCCGTCTCCC | 73 |
| 7680 | GCCACCGCCGCCGTCTCCCG | 74 |
| 7681 | CCACCGCCGCCGTCTCCCGG | 75 |
| 7682 | CACCGCCGCCGTCTCCCGGG | 76 |
| 7683 | ACCGCCGCCGTCTCCCGGGG | 77 |
| 7684 | CCGCCGCCGTCTCCCGGGGC | 78 |
| 7685 | CGCCGCCGTCTCCCGGGGCC | 79 |
| 7686 | GCCGCCGTCTCCCGGGGCCC | 80 |
| 7687 | CCGCCGTCTCCCGGGGCCCC | 81 |
| 7688 | CGCCGTCTCCCGGGGCCCCC | 82 |
| 7689 | GCCGTCTCCCGGGGCCCCCG | 83 |
| 7690 | CCGTCTCCCGGGGCCCCCGC | 84 |
| 7691 | CGTCTCCCGGGGCCCCCGCG | 85 |
| 7692 | GTCTCCCGGGGCCCCCGCGC | 86 |
| 7693 | TCTCCCGGGGCCCCCGCGCA | 87 |
| 7694 | CTCCCGGGGCCCCCGCGCAC | 88 |
| 7695 | TCCCGGGGCCCCCGCGCACG | 89 |
| 7696 | CCCGGGGCCCCCGCGCACGC | 90 |
| 7697 | CCGGGGCCCCCGCGCACGCT | 91 |
| 7698 | CGGGGCCCCCGCGCACGCTC | 92 |
| 7699 | GGGGCCCCCGCGCACGCTCC | 93 |
| 7700 | GGGCCCCCGCGCACGCTCCT | 94 |
| 7701 | GGCCCCCGCGCACGCTCCTC | 95 |
| 7702 | GCCCCCGCGCACGCTCCTCC | 96 |
| 7703 | CCCCCGCGCACGCTCCTCCG | 97 |
| 7704 | CCCCGCGCACGCTCCTCCGC | 98 |
| 7705 | CCCGCGCACGCTCCTCCGCG | 99 |
| 7706 | CCGCGCACGCTCCTCCGCGT | 100 |
| 7707 | CGCGCACGCTCCTCCGCGTG | 101 |
| 7708 | GCGCACGCTCCTCCGCGTGC | 102 |
| 7709 | CGCACGCTCCTCCGCGTGCT | 103 |
| 7710 | GCACGCTCCTCCGCGTGCTC | 104 |
| 7711 | CACGCTCCTCCGCGTGCTCT | 105 |
| 7712 | ACGCTCCTCCGCGTGCTCTC | 106 |
| 7713 | CGCTCCTCCGCGTGCTCTCG | 107 |
| 7714 | GCTCCTCCGCGTGCTCTCGC | 108 |
| 7715 | CTCCTCCGCGTGCTCTCGCC | 109 |
| 7716 | TCCTCCGCGTGCTCTCGCCT | 110 |
| 7717 | CCTCCGCGTGCTCTCGCCTA | 111 |
| 7718 | CTCCGCGTGCTCTCGCCTAC | 112 |
| 7719 | TCCGCGTGCTCTCGCCTACC | 113 |
| 7720 | CCGCGTGCTCTCGCCTACCG | 114 |
| 7721 | CGCGTGCTCTCGCCTACCGC | 115 |
| 7722 | GCGTGCTCTCGCCTACCGCT | 116 |
| 7723 | CGTGCTCTCGCCTACCGCTG | 117 |
| 7724 | GTGCTCTCGCCTACCGCTGC | 118 |
| 7725 | TGCTCTCGCCTACCGCTGCC | 119 |
| 7726 | GCTCTCGCCTACCGCTGCCG | 120 |
| 7727 | CTCTCGCCTACCGCTGCCGA | 121 |
| 7728 | TCTCGCCTACCGCTGCCGAG | 122 |
| 7729 | CTCGCCTACCGCTGCCGAGG | 123 |
| 7730 | TCGCCTACCGCTGCCGAGGA | 124 |
| 7731 | CGCCTACCGCTGCCGAGGAA | 125 |
| 7732 | GCCTACCGCTGCCGAGGAAA | 126 |
| 7733 | CCTACCGCTGCCGAGGAAAC | 127 |
| 7734 | CTACCGCTGCCGAGGAAACT | 128 |
| 7735 | TACCGCTGCCGAGGAAACTG | 129 |
| 7736 | ACCGCTGCCGAGGAAACTGA | 130 |
| 7737 | CCGCTGCCGAGGAAACTGAC | 131 |
| 7738 | CGCTGCCGAGGAAACTGACG | 132 |
| 7739 | GCTGCCGAGGAAACTGACGG | 133 |
| 7740 | CTGCCGAGGAAACTGACGGA | 134 |
| 7741 | GTGCGAGTGGGATCCGCCGCG | 34 |
| 7742 | TGCGAGTGGGATCCGCCGCG | 35 |
| 7743 | GCGAGTGGGATCCGCCGCGT | 36 |
| 7744 | CGAGTGGGATCCGCCGCGTC | 37 |
| 7745 | GAGTGGGATCCGCCGCGTCC | 38 |
| 7746 | AGTGGGATCCGCCGCGTCCT | 39 |
| 7747 | GTGGGATCCGCCGCGTCCTT | 40 |
| 7748 | TGGGATCCGCCGCGTCCTTG | 41 |
| 7749 | GGGATCCGCCGCGTCCTTGC | 42 |
| 7750 | GGATCCGCCGCGTCCTTGCT | 43 |
| 7751 | GATCCGCCGCGTCCTTGCTC | 44 |
| 7752 | ATCCGCCGCGTCCTTGCTCT | 45 |
| 7753 | TCCGCCGCGTCCTTGCTCTG | 46 |
| 7754 | CCGCCGCGTCCTTGCTCTGC | 47 |
| 7755 | CGCCGCGTCCTTGCTCTGCC | 48 |
| 7756 | GCCGCGTCCTTGCTCTGCCC | 49 |
| 7757 | CCGCGTCCTTGCTCTGCCCG | 50 |
| 7758 | CGCGTCCTTGCTCTGCCCGC | 51 |
| 7759 | GCGTCCTTGCTCTGCCCGCG | 52 |
| 7760 | CGTCCTTGCTCTGCCCGCGC | 53 |
| 7761 | GTCCTTGCTCTGCCCGCGCC | 54 |
| 7762 | TCCTTGCTCTGCCCGCGCCG | 55 |
| 7763 | CCTTGCTCTGCCCGCGCCGC | 56 |
| 7764 | CTTGCTCTGCCCGCGCCGCC | 57 |
| 7765 | TTGCTCTGCCCGCGCCGCCA | 58 |
| 7766 | TGCTCTGCCCGCGCCGCCAC | 59 |
| 7767 | GCTCTGCCCGCGCCGCCACC | 60 |
| 7768 | CTCTGCCCGCGCCGCCACCG | 61 |
| 7769 | TCTGCCCGCGCCGCCACCGC | 62 |
| 7770 | CTGCCCGCGCCGCCACCGCC | 63 |
| 7771 | TGCCCGCGCCGCCACCGCCG | 64 |
| 7772 | GCCCGCGCCGCCACCGCCGC | 65 |
| 7773 | CCCGCGCCGCCACCGCCGCC | 66 |
| 7774 | CCGCGCCGCCACCGCCGCCG | 67 |
| 7775 | CGCGCCGCCACCGCCGCCGT | 68 |
| 7776 | GCGCCGCCACCGCCGCCGTC | 69 |
| 7777 | CGCCGCCACCGCCGCCGTCT | 70 |
| 7778 | GCCGCCACCGCCGCCGTCTC | 71 |
| 7779 | CCGCCACCGCCGCCGTCTCC | 72 |
| 7780 | CGCCACCGCCGCCGTCTCCC | 73 |
| 7781 | GCCACCGCCGCCGTCTCCCG | 74 |
| 7782 | CCACCGCCGCCGTCTCCCGG | 75 |
| 7783 | CACCGCCGCCGTCTCCCGGG | 76 |
| 7784 | ACCGCCGCCGTCTCCCGGGG | 77 |
| 7785 | CCGCCGCCGTCTCCCGGGGC | 78 |
| 7786 | CGCCGCCGTCTCCCGGGGCC | 79 |
| 7787 | GCCGCCGTCTCCCGGGGCCC | 80 |
| 7788 | CCGCCGTCTCCCGGGGCCCC | 81 |
| 7789 | CGCCGTCTCCCGGGGCCCCC | 82 |
| 7790 | GCCGTCTCCCGGGGCCCCCG | 83 |
| 7791 | CCGTCTCCCGGGGCCCCCGC | 84 |
| 7792 | CGTCTCCCGGGGCCCCCGCG | 85 |
| 7793 | GTCTCCCGGGGCCCCCGCGC | 86 |
| 7794 | TCTCCCGGGGCCCCCGCGCA | 87 |
| 7795 | CTCCCGGGGCCCCCGCGCAC | 88 |
| 7796 | TCCCGGGGCCCCCGCGCACG | 89 |
| 7797 | CCCGGGGCCCCCGCGCACGC | 90 |
| 7798 | CCGGGGCCCCCGCGCACGCT | 91 |
| 7799 | CGGGGCCCCCGCGCACGCTC | 92 |
| 7800 | GGGGCCCCCGCGCACGCTCC | 93 |
| 7801 | GGGCCCCCGCGCACGCTCCT | 94 |
| 7802 | GGCCCCCGCGCACGCTCCTC | 95 |
| 7803 | GCCCCCGCGCACGCTCCTCC | 96 |
| 7804 | CCCCCGCGCACGCTCCTCCG | 97 |
| 7805 | CCCCGCGCACGCTCCTCCGC | 98 |
| 7806 | CCCGCGCACGCTCCTCCGCG | 99 |
| 7807 | CCGCGCACGCTCCTCCGCGT | 100 |
| 7808 | CGCGCACGCTCCTCCGCGTG | 101 |
| 7809 | GCGCACGCTCCTCCGCGTGC | 102 |
| 7810 | CGCACGCTCCTCCGCGTGCT | 103 |
| 7811 | GCACGCTCCTCCGCGTGCTC | 104 |
| 7812 | CACGCTCCTCCGCGTGCTCT | 105 |
| 7813 | ACGCTCCTCCGCGTGCTCTC | 106 |
| 7814 | CGCTCCTCCGCGTGCTCTCG | 107 |
| 7815 | GCTCCTCCGCGTGCTCTCGC | 108 |
| 7816 | CTCCTCCGCGTGCTCTCGCC | 109 |
| 7817 | TCCTCCGCGTGCTCTCGCCT | 110 |
| 7818 | CCTCCGCGTGCTCTCGCCTA | 111 |
| 7819 | CTCCGCGTGCTCTCGCCTAC | 112 |
| 7820 | TCCGCGTGCTCTCGCCTACC | 113 |
| 7821 | CCGCGTGCTCTCGCCTACCG | 114 |
| 7822 | CGCGTGCTCTCGCCTACCGC | 115 |
| 7823 | GCGTGCTCTCGCCTACCGCT | 116 |
| 7824 | CGTGCTCTCGCCTACCGCTG | 117 |
| 7825 | GTGCTCTCGCCTACCGCTGC | 118 |
| 7826 | TGCTCTCGCCTACCGCTGCC | 119 |
| 7827 | GCTCTCGCCTACCGCTGCCG | 120 |
| 7828 | CTCTCGCCTACCGCTGCCGA | 121 |
| 7829 | TCTCGCCTACCGCTGCCGAG | 122 |
| 7830 | CTCGCCTACCGCTGCCGAGG | 123 |
| 7831 | TCGCCTACCGCTGCCGAGGA | 124 |
| 7832 | CGCCTACCGCTGCCGAGGAA | 125 |
| 7833 | GCCTACCGCTGCCGAGGAAA | 126 |
| 7834 | CCTACCGCTGCCGAGGAAAC | 127 |
| 7835 | CTACCGCTGCCGAGGAAACT | 128 |
| 7836 | TACCGCTGCCGAGGAAACTG | 129 |
| 7837 | ACCGCTGCCGAGGAAACTGA | 130 |
| 7838 | CCGCTGCCGAGGAAACTGAC | 131 |
| 7839 | CGCTGCCGAGGAAACTGACG | 132 |
| 7840 | GCTGCCGAGGAAACTGACGG | 133 |
| 7841 | CTGCCGAGGAAACTGACGGA | 134 |
| 7842 | TGTGCGAGTGGGATCCGCCG | 33 |
| 7843 | CTGTGCGAGTGGGATCCGCC | 32 |
| 7844 | GCTGTGCGAGTGGGATCCGC | 31 |
| 7845 | TGCTGTGCGAGTGGGATCCG | 30 |
| 7846 | CTGCTGTGCGAGTGGGATCC | 29 |
| 7847 | GCTGCTGTGCGAGTGGGATC | 28 |
| 7848 | CGCTGCTGTGCGAGTGGGAT | 27 |
| 7849 | GCGCTGCTGTGCGAGTGGGA | 26 |
| 7850 | TGCGCTGCTGTGCGAGTGGG | 25 |
| 7851 | GTGCGCTGCTGTGCGAGTGG | 24 |
| 7852 | AGTGCGCTGCTGTGCGAGTG | 23 |
| 7853 | GAGTGCGCTGCTGTGCGAGT | 22 |
| 7854 | CGAGTGCGCTGCTGTGCGAG | 21 |
| 7855 | CCGAGTGCGCTGCTGTGCGA | 20 |
| 7856 | ACCGAGTGCGCTGCTGTGCG | 19 |
| 7857 | CACCGAGTGCGCTGCTGTGC | 18 |
| 7858 | GCACCGAGTGCGCTGCTGTG | 17 |
| 7859 | GGCACCGAGTGCGCTGCTGT | 16 |
| 7860 | GGGCACCGAGTGCGCTGCTG | 15 |
| 7861 | GGGGCACCGAGTGCGCTGCT | 14 |
| 7862 | CGGGGCACCGAGTGCGCTGC | 13 |
| 7863 | GCGGGGCACCGAGTGCGCTG | 12 |
| 7864 | CGCGGGGCACCGAGTGCGCT | 11 |
| 7865 | GCGCGGGGCACCGAGTGCGC | 10 |
| 7866 | TGCGCGGGGCACCGAGTGCG | 9 |
| 7867 | CTGCGCGGGGCACCGAGTGC | 8 |
| 7868 | CCTGCGCGGGGCACCGAGTG | 7 |
| 7869 | CCCTGCGCGGGGCACCGAGT | 6 |
| 7870 | ACCCTGCGCGGGGCACCGAG | 5 |
| 7871 | GACCCTGCGCGGGGCACCGA | 4 |
| 7872 | CGACCCTGCGCGGGGCACCG | 3 |
| 7873 | GCGACCCTGCGCGGGGCACC | 2 |
| 7874 | CGCGACCCTGCGCGGGGCAC | 1 |
| 7875 | CGCGCCGCCACCGCCGCCGTCTCCCGG | 68 |
| 7876 | GCGCCGCCACCGCCGCCGTC | 69 |
| 7877 | CGCCGCCACCGCCGCCGTCT | 70 |
| 7878 | GCCGCCACCGCCGCCGTCTC | 71 |
| 7879 | CCGCCACCGCCGCCGTCTCC | 72 |
| 7880 | CGCCACCGCCGCCGTCTCCC | 73 |
| 7881 | GCCACCGCCGCCGTCTCCCG | 74 |
| 7882 | CCACCGCCGCCGTCTCCCGG | 75 |
| 7883 | CACCGCCGCCGTCTCCCGGG | 76 |
| 7884 | ACCGCCGCCGTCTCCCGGGG | 77 |
| 7885 | CCGCCGCCGTCTCCCGGGGC | 78 |
| 7886 | CGCCGCCGTCTCCCGGGGCC | 79 |
| 7887 | GCCGCCGTCTCCCGGGGCCC | 80 |
| 7888 | CCGCCGTCTCCCGGGGCCCC | 81 |
| 7889 | CGCCGTCTCCCGGGGCCCCC | 82 |
| 7890 | GCCGTCTCCCGGGGCCCCCG | 83 |
| 7891 | CCGTCTCCCGGGGCCCCCGC | 84 |
| 7892 | CGTCTCCCGGGGCCCCCGCG | 85 |
| 7893 | GTCTCCCGGGGCCCCCGCGC | 86 |
| 7894 | TCTCCCGGGGCCCCCGCGCA | 87 |
| 7895 | CTCCCGGGGCCCCCGCGCAC | 88 |
| 7896 | TCCCGGGGCCCCCGCGCACG | 89 |
| 7897 | CCCGGGGCCCCCGCGCACGC | 90 |
| 7898 | CCGGGGCCCCCGCGCACGCT | 91 |
| 7899 | CGGGGCCCCCGCGCACGCTC | 92 |
| 7900 | GGGGCCCCCGCGCACGCTCC | 93 |
| 7901 | GGGCCCCCGCGCACGCTCCT | 94 |
| 7902 | GGCCCCCGCGCACGCTCCTC | 95 |
| 7903 | GCCCCCGCGCACGCTCCTCC | 96 |
| 7904 | CCCCCGCGCACGCTCCTCCG | 97 |
| 7905 | CCCCGCGCACGCTCCTCCGC | 98 |
| 7906 | CCCGCGCACGCTCCTCCGCG | 99 |
| 7907 | CCGCGCACGCTCCTCCGCGT | 100 |
| 7908 | CGCGCACGCTCCTCCGCGTG | 101 |
| 7909 | GCGCACGCTCCTCCGCGTGC | 102 |
| 7910 | CGCACGCTCCTCCGCGTGCT | 103 |
| 7911 | GCACGCTCCTCCGCGTGCTC | 104 |
| 7912 | CACGCTCCTCCGCGTGCTCT | 105 |
| 7913 | ACGCTCCTCCGCGTGCTCTC | 106 |
| 7914 | CGCTCCTCCGCGTGCTCTCG | 107 |
| 7915 | GCTCCTCCGCGTGCTCTCGC | 108 |
| 7916 | CTCCTCCGCGTGCTCTCGCC | 109 |
| 7917 | TCCTCCGCGTGCTCTCGCCT | 110 |
| 7918 | CCTCCGCGTGCTCTCGCCTA | 111 |
| 7919 | CTCCGCGTGCTCTCGCCTAC | 112 |
| 7920 | TCCGCGTGCTCTCGCCTACC | 113 |
| 7921 | CCGCGTGCTCTCGCCTACCG | 114 |
| 7922 | CGCGTGCTCTCGCCTACCGC | 115 |
| 7923 | GCGTGCTCTCGCCTACCGCT | 116 |
| 7924 | CGTGCTCTCGCCTACCGCTG | 117 |
| 7925 | GTGCTCTCGCCTACCGCTGC | 118 |
| 7926 | TGCTCTCGCCTACCGCTGCC | 119 |
| 7927 | GCTCTCGCCTACCGCTGCCG | 120 |
| 7928 | CTCTCGCCTACCGCTGCCGA | 121 |
| 7929 | TCTCGCCTACCGCTGCCGAG | 122 |
| 7930 | CTCGCCTACCGCTGCCGAGG | 123 |
| 7931 | TCGCCTACCGCTGCCGAGGA | 124 |
| 7932 | CGCCTACCGCTGCCGAGGAA | 125 |
| 7933 | GCCTACCGCTGCCGAGGAAA | 126 |
| 7934 | CCTACCGCTGCCGAGGAAAC | 127 |
| 7935 | CTACCGCTGCCGAGGAAACT | 128 |
| 7936 | TACCGCTGCCGAGGAAACTG | 129 |
| 7937 | ACCGCTGCCGAGGAAACTGA | 130 |
| 7938 | CCGCTGCCGAGGAAACTGAC | 131 |
| 7939 | CGCTGCCGAGGAAACTGACG | 132 |
| 7940 | GCTGCCGAGGAAACTGACGG | 133 |
| 7941 | CTGCCGAGGAAACTGACGGA | 134 |
| 7942 | CCGCGCCGCCACCGCCGCCG | 67 |
| 7943 | CCCGCGCCGCCACCGCCGCC | 66 |
| 7944 | GCCCGCGCCGCCACCGCCGC | 65 |
| 7945 | TGCCCGCGCCGCCACCGCCG | 64 |
| 7946 | CTGCCCGCGCCGCCACCGCC | 63 |
| 7947 | TCTGCCCGCGCCGCCACCGC | 62 |
| 7948 | CTCTGCCCGCGCCGCCACCG | 61 |
| 7949 | GCTCTGCCCGCGCCGCCACC | 60 |
| 7950 | TGCTCTGCCCGCGCCGCCAC | 59 |
| 7951 | TTGCTCTGCCCGCGCCGCCA | 58 |
| 7952 | CTTGCTCTGCCCGCGCCGCC | 57 |
| 7953 | CCTTGCTCTGCCCGCGCCGC | 56 |
| 7954 | TCCTTGCTCTGCCCGCGCCG | 55 |
| 7955 | GTCCTTGCTCTGCCCGCGCC | 54 |
| 7956 | CGTCCTTGCTCTGCCCGCGC | 53 |
| 7957 | GCGTCCTTGCTCTGCCCGCG | 52 |
| 7958 | CGCGTCCTTGCTCTGCCCGC | 51 |
| 7959 | CCGCGTCCTTGCTCTGCCCG | 50 |
| 7960 | GCCGCGTCCTTGCTCTGCCC | 49 |
| 7961 | CGCCGCGTCCTTGCTCTGCC | 48 |
| 7962 | CCGCCGCGTCCTTGCTCTGC | 47 |
| 7963 | TCCGCCGCGTCCTTGCTCTG | 46 |
| 7964 | ATCCGCCGCGTCCTTGCTCT | 45 |
| 7965 | GATCCGCCGCGTCCTTGCTC | 44 |
| 7966 | GGATCCGCCGCGTCCTTGCT | 43 |
| 7967 | GGGATCCGCCGCGTCCTTGC | 42 |
| 7968 | TGGGATCCGCCGCGTCCTTG | 41 |
| 7969 | GTGGGATCCGCCGCGTCCTT | 40 |
| 7970 | AGTGGGATCCGCCGCGTCCT | 39 |
| 7971 | GAGTGGGATCCGCCGCGTCC | 38 |
| 7972 | CGAGTGGGATCCGCCGCGTC | 37 |
| 7973 | GCGAGTGGGATCCGCCGCGT | 36 |
| 7974 | TGCGAGTGGGATCCGCCGCG | 35 |
| 7975 | GTGCGAGTGGGATCCGCCGC | 34 |
| 7976 | TGTGCGAGTGGGATCCGCCG | 33 |
| 7977 | CTGTGCGAGTGGGATCCGCC | 32 |
| 7978 | GCTGTGCGAGTGGGATCCGC | 31 |
| 7979 | TGCTGTGCGAGTGGGATCCG | 30 |
| 7980 | CTGCTGTGCGAGTGGGATCC | 29 |
| 7981 | GCTGCTGTGCGAGTGGGATC | 28 |
| 7982 | CGCTGCTGTGCGAGTGGGAT | 27 |
| 7983 | GCGCTGCTGTGCGAGTGGGA | 26 |
| 7984 | TGCGCTGCTGTGCGAGTGGG | 25 |
| 7985 | GTGCGCTGCTGTGCGAGTGG | 24 |
| 7986 | AGTGCGCTGCTGTGCGAGTG | 23 |
| 7987 | GAGTGCGCTGCTGTGCGAGT | 22 |
| 7988 | CGAGTGCGCTGCTGTGCGAG | 21 |
| 7989 | CCGAGTGCGCTGCTGTGCGA | 20 |
| 7990 | ACCGAGTGCGCTGCTGTGCG | 19 |
| 7991 | CACCGAGTGCGCTGCTGTGC | 18 |
| 7992 | GCACCGAGTGCGCTGCTGTG | 17 |
| 7993 | GGCACCGAGTGCGCTGCTGT | 16 |
| 7994 | GGGCACCGAGTGCGCTGCTG | 15 |
| 7995 | GGGGCACCGAGTGCGCTGCT | 14 |
| 7996 | CGGGGCACCGAGTGCGCTGC | 13 |
| 7997 | GCGGGGCACCGAGTGCGCTG | 12 |
| 7998 | CGCGGGGCACCGAGTGCGCT | 11 |
| 7999 | GCGCGGGGCACCGAGTGCGC | 10 |
| 8000 | TGCGCGGGGCACCGAGTGCG | 9 |
| 8001 | CTGCGCGGGGCACCGAGTGC | 8 |
| 8002 | CCTGCGCGGGGCACCGAGTG | 7 |
| 8003 | CCCTGCGCGGGGCACCGAGT | 6 |
| 8004 | ACCCTGCGCGGGGCACCGAG | 5 |
| 8005 | GACCCTGCGCGGGGCACCGA | 4 |
| 8006 | CGACCCTGCGCGGGGCACCG | 3 |
| 8007 | GCGACCCTGCGCGGGGCACC | 2 |
| 8008 | CGCGACCCTGCGCGGGGCAC | 1 |
| 8009 | CGCGCACGCTCCTCCGCGTGCTCTCG | 101 |
| 8010 | GCGCACGCTCCTCCGCGTGC | 102 |
| 8011 | CGCACGCTCCTCCGCGTGCT | 103 |
| 8012 | GCACGCTCCTCCGCGTGCTC | 104 |
| 8013 | CACGCTCCTCCGCGTGCTCT | 105 |
| 8014 | ACGCTCCTCCGCGTGCTCTC | 106 |
| 8015 | CGCTCCTCCGCGTGCTCTCG | 107 |
| 8016 | GCTCCTCCGCGTGCTCTCGC | 108 |
| 8017 | CTCCTCCGCGTGCTCTCGCC | 109 |
| 8018 | TCCTCCGCGTGCTCTCGCCT | 110 |
| 8019 | CCTCCGCGTGCTCTCGCCTA | 111 |
| 8020 | CTCCGCGTGCTCTCGCCTAC | 112 |
| 8021 | TCCGCGTGCTCTCGCCTACC | 113 |
| 8022 | CCGCGTGCTCTCGCCTACCG | 114 |
| 8023 | CGCGTGCTCTCGCCTACCGC | 115 |
| 8024 | GCGTGCTCTCGCCTACCGCT | 116 |
| 8025 | CGTGCTCTCGCCTACCGCTG | 117 |
| 8026 | GTGCTCTCGCCTACCGCTGC | 118 |
| 8027 | TGCTCTCGCCTACCGCTGCC | 119 |
| 8028 | GCTCTCGCCTACCGCTGCCG | 120 |
| 8029 | CTCTCGCCTACCGCTGCCGA | 121 |
| 8030 | TCTCGCCTACCGCTGCCGAG | 122 |
| 8031 | CTCGCCTACCGCTGCCGAGG | 123 |
| 8032 | TCGCCTACCGCTGCCGAGGA | 124 |
| 8033 | CGCCTACCGCTGCCGAGGAA | 125 |
| 8034 | GCCTACCGCTGCCGAGGAAA | 126 |
| 8035 | CCTACCGCTGCCGAGGAAAC | 127 |
| 8036 | CTACCGCTGCCGAGGAAACT | 128 |
| 8037 | TACCGCTGCCGAGGAAACTG | 129 |
| 8038 | ACCGCTGCCGAGGAAACTGA | 130 |
| 8039 | CCGCTGCCGAGGAAACTGAC | 131 |
| 8040 | CGCTGCCGAGGAAACTGACG | 132 |
| 8041 | GCTGCCGAGGAAACTGACGG | 133 |
| 8042 | CTGCCGAGGAAACTGACGGA | 134 |
| 8043 | CCGCGCACGCTCCTCCGCGT | 100 |
| 8044 | CCCGCGCACGCTCCTCCGCG | 99 |
| 8045 | CCCCGCGCACGCTCCTCCGC | 98 |
| 8046 | CCCCCGCGCACGCTCCTCCG | 97 |
| 8047 | GCCCCCGCGCACGCTCCTCC | 96 |
| 8048 | GGCCCCCGCGCACGCTCCTC | 95 |
| 8049 | GGGCCCCCGCGCACGCTCCT | 94 |
| 8050 | GGGGCCCCCGCGCACGCTCC | 93 |
| 8051 | CGGGGCCCCCGCGCACGCTC | 92 |
| 8052 | CCGGGGCCCCCGCGCACGCT | 91 |
| 8053 | CCCGGGGCCCCCGCGCACGC | 90 |
| 8054 | TCCCGGGGCCCCCGCGCACG | 89 |
| 8055 | CTCCCGGGGCCCCCGCGCAC | 88 |
| 8056 | TCTCCCGGGGCCCCCGCGCA | 87 |
| 8057 | GTCTCCCGGGGCCCCCGCGC | 86 |
| 8058 | CGTCTCCCGGGGCCCCCGCG | 85 |
| 8059 | CCGTCTCCCGGGGCCCCCGC | 84 |
| 8060 | GCCGTCTCCCGGGGCCCCCG | 83 |
| 8061 | CGCCGTCTCCCGGGGCCCCC | 82 |
| 8062 | CCGCCGTCTCCCGGGGCCCC | 81 |
| 8063 | GCCGCCGTCTCCCGGGGCCC | 80 |
| 8064 | CGCCGCCGTCTCCCGGGGCC | 79 |
| 8065 | CCGCCGCCGTCTCCCGGGGC | 78 |
| 8066 | ACCGCCGCCGTCTCCCGGGG | 77 |
| 8067 | CACCGCCGCCGTCTCCCGGG | 76 |
| 8068 | CCACCGCCGCCGTCTCCCGG | 75 |
| 8069 | GCCACCGCCGCCGTCTCCCG | 74 |
| 8070 | CGCCACCGCCGCCGTCTCCC | 73 |
| 8071 | CCGCCACCGCCGCCGTCTCC | 72 |
| 8072 | GCCGCCACCGCCGCCGTCTC | 71 |
| 8073 | CGCCGCCACCGCCGCCGTCT | 70 |
| 8074 | GCGCCGCCACCGCCGCCGTC | 69 |
| 8075 | CGCGCCGCCACCGCCGCCGT | 68 |
| 8076 | CCGCGCCGCCACCGCCGCCG | 67 |
| 8077 | CCCGCGCCGCCACCGCCGCC | 66 |
| 8078 | GCCCGCGCCGCCACCGCCGC | 65 |
| 8079 | TGCCCGCGCCGCCACCGCCG | 64 |
| 8080 | CTGCCCGCGCCGCCACCGCC | 63 |
| 8081 | TCTGCCCGCGCCGCCACCGC | 62 |
| 8082 | CTCTGCCCGCGCCGCCACCG | 61 |
| 8083 | GCTCTGCCCGCGCCGCCACC | 60 |
| 8084 | TGCTCTGCCCGCGCCGCCAC | 59 |
| 8085 | TTGCTCTGCCCGCGCCGCCA | 58 |
| 8086 | CTTGCTCTGCCCGCGCCGCC | 57 |
| 8087 | CCTTGCTCTGCCCGCGCCGC | 56 |
| 8088 | TCCTTGCTCTGCCCGCGCCG | 55 |
| 8089 | GTCCTTGCTCTGCCCGCGCC | 54 |
| 8090 | CGTCCTTGCTCTGCCCGCGC | 53 |
| 8091 | GCGTCCTTGCTCTGCCCGCG | 52 |
| 8092 | CGCGTCCTTGCTCTGCCCGC | 51 |
| 8093 | CCGCGTCCTTGCTCTGCCCG | 50 |
| 8094 | GCCGCGTCCTTGCTCTGCCC | 49 |
| 8095 | CGCCGCGTCCTTGCTCTGCC | 48 |
| 8096 | CCGCCGCGTCCTTGCTCTGC | 47 |
| 8097 | TCCGCCGCGTCCTTGCTCTG | 46 |
| 8098 | ATCCGCCGCGTCCTTGCTCT | 45 |
| 8099 | GATCCGCCGCGTCCTTGCTC | 44 |
| 8100 | GGATCCGCCGCGTCCTTGCT | 43 |
| 8101 | GGGATCCGCCGCGTCCTTGC | 42 |
| 8102 | TGGGATCCGCCGCGTCCTTG | 41 |
| 8103 | GTGGGATCCGCCGCGTCCTT | 40 |
| 8104 | AGTGGGATCCGCCGCGTCCT | 39 |
| 8105 | GAGTGGGATCCGCCGCGTCC | 38 |
| 8106 | CGAGTGGGATCCGCCGCGTC | 37 |
| 8107 | GCGAGTGGGATCCGCCGCGT | 36 |
| 8108 | TGCGAGTGGGATCCGCCGCG | 35 |
| 8109 | GTGCGAGTGGGATCCGCCGC | 34 |
| 8110 | TGTGCGAGTGGGATCCGCCG | 33 |
| 8111 | CTGTGCGAGTGGGATCCGCC | 32 |
| 8112 | GCTGTGCGAGTGGGATCCGC | 31 |
| 8113 | TGCTGTGCGAGTGGGATCCG | 30 |
| 8114 | CTGCTGTGCGAGTGGGATCC | 29 |
| 8115 | GCTGCTGTGCGAGTGGGATC | 28 |
| 8116 | CGCTGCTGTGCGAGTGGGAT | 27 |
| 8117 | GCGCTGCTGTGCGAGTGGGA | 26 |
| 8118 | TGCGCTGCTGTGCGAGTGGG | 25 |
| 8119 | GTGCGCTGCTGTGCGAGTGG | 24 |
| 8120 | AGTGCGCTGCTGTGCGAGTG | 23 |
| 8121 | GAGTGCGCTGCTGTGCGAGT | 22 |
| 8122 | CGAGTGCGCTGCTGTGCGAG | 21 |
| 8123 | CCGAGTGCGCTGCTGTGCGA | 20 |
| 8124 | ACCGAGTGCGCTGCTGTGCG | 19 |
| 8125 | CACCGAGTGCGCTGCTGTGC | 18 |
| 8126 | GCACCGAGTGCGCTGCTGTG | 17 |
| 8127 | GGCACCGAGTGCGCTGCTGT | 16 |
| 8128 | GGGCACCGAGTGCGCTGCTG | 15 |
| 8129 | GGGGCACCGAGTGCGCTGCT | 14 |
| 8130 | CGGGGCACCGAGTGCGCTGC | 13 |
| 8131 | GCGGGGCACCGAGTGCGCTG | 12 |
| 8132 | CGCGGGGCACCGAGTGCGCT | 11 |
| 8133 | GCGCGGGGCACCGAGTGCGC | 10 |
| 8134 | TGCGCGGGGCACCGAGTGCG | 9 |
| 8135 | CTGCGCGGGGCACCGAGTGC | 8 |
| 8136 | CCTGCGCGGGGCACCGAGTG | 7 |
| 8137 | CCCTGCGCGGGGCACCGAGT | 6 |
| 8138 | ACCCTGCGCGGGGCACCGAG | 5 |
| 8139 | GACCCTGCGCGGGGCACCGA | 4 |
| 8140 | CGACCCTGCGCGGGGCACCG | 3 |
| 8141 | GCGACCCTGCGCGGGGCACC | 2 |
| 8142 | CGCGACCCTGCGCGGGGCAC | 1 |
| 8143 | CCGAGGAAACTGACGGAGCCCGAGCGCGG | 137 |
| 8144 | CGAGGAAACTGACGGAGCCC | 138 |
| 8145 | CGAGTCAGCTGATCCGGCCCACCCCG | 186 |
| 8146 | GAGTCAGCTGATCCGGCCCA | 187 |
| 8147 | AGTCAGCTGATCCGGCCCAC | 188 |
| 8148 | GTCAGCTGATCCGGCCCACC | 189 |
| 8149 | TCAGCTGATCCGGCCCACCC | 190 |
| 8150 | CAGCTGATCCGGCCCACCCC | 191 |
| 8151 | AGCTGATCCGGCCCACCCCG | 192 |
| 8152 | GCTGATCCGGCCCACCCCGC | 193 |
| 8153 | CTGATCCGGCCCACCCCGCT | 194 |
| 8154 | TGATCCGGCCCACCCCGCTC | 195 |
| 8155 | GATCCGGCCCACCCCGCTCG | 196 |
| 8156 | ATCCGGCCCACCCCGCTCGG | 197 |
| 8157 | TCCGGCCCACCCCGCTCGGC | 198 |
| 8158 | CCGGCCCACCCCGCTCGGCA | 199 |
| 8159 | CGGCCCACCCCGCTCGGCAC | 200 |
| 8160 | GGCCCACCCCGCTCGGCACC | 201 |
| 8161 | GCCCACCCCGCTCGGCACCC | 202 |
| 8162 | CCCACCCCGCTCGGCACCCG | 203 |
| 8163 | CCACCCCGCTCGGCACCCGA | 204 |
| 8164 | CACCCCGCTCGGCACCCGAG | 205 |
| 8165 | ACCCCGCTCGGCACCCGAGA | 206 |
| 8166 | CCCCGCTCGGCACCCGAGAG | 207 |
| 8167 | CCCGCTCGGCACCCGAGAGA | 208 |
| 8168 | CCGCTCGGCACCCGAGAGAG | 209 |
| 8169 | CGCTCGGCACCCGAGAGAGA | 210 |
| 8170 | GCTCGGCACCCGAGAGAGAC | 211 |
| 8171 | CTCGGCACCCGAGAGAGACC | 212 |
| 8172 | TCGGCACCCGAGAGAGACCC | 213 |
| 8173 | CGGCACCCGAGAGAGACCCC | 214 |
| 8174 | GGCACCCGAGAGAGACCCCT | 215 |
| 8175 | GCACCCGAGAGAGACCCCTA | 216 |
| 8176 | CACCCGAGAGAGACCCCTAG | 217 |
| 8177 | ACCCGAGAGAGACCCCTAGC | 218 |
| 8178 | CCCGAGAGAGACCCCTAGCG | 219 |
| 8179 | CCGAGAGAGACCCCTAGCGG | 220 |
| 8180 | CGAGAGAGACCCCTAGCGGC | 221 |
| 8181 | GAGAGAGACCCCTAGCGGCG | 222 |
| 8182 | AGAGAGACCCCTAGCGGCGC | 223 |
| 8183 | GAGAGACCCCTAGCGGCGCC | 224 |
| 8184 | AGAGACCCCTAGCGGCGCCG | 225 |
| 8185 | GAGACCCCTAGCGGCGCCGC | 226 |
| 8186 | AGACCCCTAGCGGCGCCGCC | 227 |
| 8187 | GACCCCTAGCGGCGCCGCCG | 228 |
| 8188 | ACCCCTAGCGGCGCCGCCGG | 229 |
| 8189 | CCCCTAGCGGCGCCGCCGGG | 230 |
| 8190 | CCCTAGCGGCGCCGCCGGGG | 231 |
| 8191 | CCTAGCGGCGCCGCCGGGGA | 232 |
| 8192 | CTAGCGGCGCCGCCGGGGAA | 233 |
| 8193 | TAGCGGCGCCGCCGGGGAAC | 234 |
| 8194 | AGCGGCGCCGCCGGGGAACT | 235 |
| 8195 | GCGGCGCCGCCGGGGAACTG | 236 |
| 8196 | CGGCGCCGCCGGGGAACTGC | 237 |
| 8197 | GGCGCCGCCGGGGAACTGCG | 238 |
| 8198 | GCGCCGCCGGGGAACTGCGC | 239 |
| 8199 | CGCCGCCGGGGAACTGCGCC | 240 |
| 8200 | GCCGCCGGGGAACTGCGCCC | 241 |
| 8201 | CCGCCGGGGAACTGCGCCCG | 242 |
| 8202 | CGCCGGGGAACTGCGCCCGC | 243 |
| 8203 | GCCGGGGAACTGCGCCCGCT | 244 |
| 8204 | CCGGGGAACTGCGCCCGCTC | 245 |
| 8205 | CGGGGAACTGCGCCCGCTCG | 246 |
| 8206 | GGGGAACTGCGCCCGCTCGC | 247 |
| 8207 | GGGAACTGCGCCCGCTCGCG | 248 |
| 8208 | GGAACTGCGCCCGCTCGCGC | 249 |
| 8209 | GAACTGCGCCCGCTCGCGCC | 250 |
| 8210 | AACTGCGCCCGCTCGCGCCG | 251 |
| 8211 | ACTGCGCCCGCTCGCGCCGG | 252 |
| 8212 | CTGCGCCCGCTCGCGCCGGG | 253 |
| 8213 | TGCGCCCGCTCGCGCCGGGA | 254 |
| 8214 | GCGCCCGCTCGCGCCGGGAG | 255 |
| 8215 | CGCCCGCTCGCGCCGGGAGG | 256 |
| 8216 | GCCCGCTCGCGCCGGGAGGG | 257 |
| 8217 | CCCGCTCGCGCCGGGAGGGG | 258 |
| 8218 | CCGCTCGCGCCGGGAGGGGC | 259 |
| 8219 | CGCTCGCGCCGGGAGGGGCC | 260 |
| 8220 | GCTCGCGCCGGGAGGGGCCC | 261 |
| 8221 | CTCGCGCCGGGAGGGGCCCT | 262 |
| 8222 | TCGCGCCGGGAGGGGCCCTC | 263 |
| 8223 | CGCGCCGGGAGGGGCCCTCG | 264 |
| 8224 | GCGCCGGGAGGGGCCCTCGC | 265 |
| 8225 | CGCCGGGAGGGGCCCTCGCG | 266 |
| 8226 | GCCGGGAGGGGCCCTCGCGC | 267 |
| 8227 | CCGGGAGGGGCCCTCGCGCC | 268 |
| 8228 | CGGGAGGGGCCCTCGCGCCC | 269 |
| 8229 | GGGAGGGGCCCTCGCGCCCC | 270 |
| 8230 | GGAGGGGCCCTCGCGCCCCG | 271 |
| 8231 | GAGGGGCCCTCGCGCCCCGC | 272 |
| 8232 | AGGGGCCCTCGCGCCCCGCG | 273 |
| 8233 | GGGGCCCTCGCGCCCCGCGC | 274 |
| 8234 | GGGCCCTCGCGCCCCGCGCC | 275 |
| 8235 | GGCCCTCGCGCCCCGCGCCC | 276 |
| 8236 | GCCCTCGCGCCCCGCGCCCA | 277 |
| 8237 | CCCTCGCGCCCCGCGCCCAC | 278 |
| 8238 | CCTCGCGCCCCGCGCCCACA | 279 |
| 8239 | CTCGCGCCCCGCGCCCACAG | 280 |
| 8240 | TCGCGCCCCGCGCCCACAGG | 281 |
| 8241 | CGCGCCCCGCGCCCACAGGT | 282 |
| 8242 | GCGCCCCGCGCCCACAGGTG | 283 |
| 8243 | CGCCCCGCGCCCACAGGTGC | 284 |
| 8244 | GCCCCGCGCCCACAGGTGCA | 285 |
| 8245 | CCCCGCGCCCACAGGTGCAC | 286 |
| 8246 | CCCGCGCCCACAGGTGCACG | 287 |
| 8247 | CCGCGCCCACAGGTGCACGC | 288 |
| 8248 | CGCGCCCACAGGTGCACGCG | 289 |
| 8249 | GCGCCCACAGGTGCACGCGC | 290 |
| 8250 | CGCCCACAGGTGCACGCGCC | 291 |
| 8251 | GCCCACAGGTGCACGCGCCC | 292 |
| 8252 | CCCACAGGTGCACGCGCCCT | 293 |
| 8253 | CCACAGGTGCACGCGCCCTT | 294 |
| 8254 | CACAGGTGCACGCGCCCTTG | 295 |
| 8255 | ACAGGTGCACGCGCCCTTGG | 296 |
| 8256 | CAGGTGCACGCGCCCTTGGC | 297 |
| 8257 | AGGTGCACGCGCCCTTGGCG | 298 |
| 8258 | GGTGCACGCGCCCTTGGCGC | 299 |
| 8259 | GTGCACGCGCCCTTGGCGCC | 300 |
| 8260 | TGCACGCGCCCTTGGCGCCG | 301 |
| 8261 | GCACGCGCCCTTGGCGCCGC | 302 |
| 8262 | CACGCGCCCTTGGCGCCGCC | 303 |
| 8263 | ACGCGCCCTTGGCGCCGCCT | 304 |
| 8264 | CGCGCCCTTGGCGCCGCCTG | 305 |
| 8265 | GCGCCCTTGGCGCCGCCTGC | 306 |
| 8266 | CGCCCTTGGCGCCGCCTGCA | 307 |
| 8267 | GCCCTTGGCGCCGCCTGCAC | 308 |
| 8268 | CCCTTGGCGCCGCCTGCACC | 309 |
| 8269 | CCTTGGCGCCGCCTGCACCC | 310 |
| 8270 | CTTGGCGCCGCCTGCACCCC | 311 |
| 8271 | TTGGCGCCGCCTGCACCCCA | 312 |
| 8272 | TGGCGCCGCCTGCACCCCAC | 313 |
| 8273 | GGCGCCGCCTGCACCCCACG | 314 |
| 8274 | GCGCCGCCTGCACCCCACGC | 315 |
| 8275 | CGCCGCCTGCACCCCACGCG | 316 |
| 8276 | GCCGCCTGCACCCCACGCGC | 317 |
| 8277 | CCGCCTGCACCCCACGCGCC | 318 |
| 8278 | CGCCTGCACCCCACGCGCCC | 319 |
| 8279 | GCCTGCACCCCACGCGCCCC | 320 |
| 8280 | CCTGCACCCCACGCGCCCCC | 321 |
| 8281 | CTGCACCCCACGCGCCCCCT | 322 |
| 8282 | TGCACCCCACGCGCCCCCTC | 323 |
| 8283 | GCACCCCACGCGCCCCCTCC | 324 |
| 8284 | CACCCCACGCGCCCCCTCCG | 325 |
| 8285 | ACCCCACGCGCCCCCTCCGC | 326 |
| 8286 | CCCCACGCGCCCCCTCCGCT | 327 |
| 8287 | CCCACGCGCCCCCTCCGCTC | 328 |
| 8288 | CCACGCGCCCCCTCCGCTCC | 329 |
| 8289 | CACGCGCCCCCTCCGCTCCC | 330 |
| 8290 | ACGCGCCCCCTCCGCTCCCC | 331 |
| 8291 | CGCGCCCCCTCCGCTCCCCG | 332 |
| 8292 | GCGCCCCCTCCGCTCCCCGG | 333 |
| 8293 | CGCCCCCTCCGCTCCCCGGC | 334 |
| 8294 | GCCCCCTCCGCTCCCCGGCC | 335 |
| 8295 | GCGAGTCAGCTGATCCGGCC | 185 |
| 8296 | GGCGAGTCAGCTGATCCGGC | 184 |
| 8297 | AGGCGAGTCAGCTGATCCGG | 183 |
| 8298 | CAGGCGAGTCAGCTGATCCG | 182 |
| 8299 | CCAGGCGAGTCAGCTGATCC | 181 |
| 8300 | GCCAGGCGAGTCAGCTGATC | 180 |
| 8301 | AGCCAGGCGAGTCAGCTGAT | 179 |
| 8302 | GAGCCAGGCGAGTCAGCTGA | 178 |
| 8303 | AGAGCCAGGCGAGTCAGCTG | 177 |
| 8304 | CAGAGCCAGGCGAGTCAGCT | 176 |
| 8305 | TCAGAGCCAGGCGAGTCAGC | 175 |
| 8306 | CTCAGAGCCAGGCGAGTCAG | 174 |
| 8307 | GCTCAGAGCCAGGCGAGTCA | 173 |
| 8308 | GGCTCAGAGCCAGGCGAGTC | 172 |
| 8309 | GGGCTCAGAGCCAGGCGAGT | 171 |
| 8310 | CGAGAGAGACCCCTAGCGGCGCCG | 221 |
| 8311 | GAGAGAGACCCCTAGCGGCG | 222 |
| 8312 | AGAGAGACCCCTAGCGGCGC | 223 |
| 8313 | GAGAGACCCCTAGCGGCGCC | 224 |
| 8314 | AGAGACCCCTAGCGGCGCCG | 225 |
| 8315 | GAGACCCCTAGCGGCGCCGC | 226 |
| 8316 | AGACCCCTAGCGGCGCCGCC | 227 |
| 8317 | GACCCCTAGCGGCGCCGCCG | 228 |
| 8318 | ACCCCTAGCGGCGCCGCCGG | 229 |
| 8319 | CCCCTAGCGGCGCCGCCGGG | 230 |
| 8320 | CCCTAGCGGCGCCGCCGGGG | 231 |
| 8321 | CCTAGCGGCGCCGCCGGGGA | 232 |
| 8322 | CTAGCGGCGCCGCCGGGGAA | 233 |
| 8323 | TAGCGGCGCCGCCGGGGAAC | 234 |
| 8324 | AGCGGCGCCGCCGGGGAACT | 235 |
| 8325 | GCGGCGCCGCCGGGGAACTG | 236 |
| 8326 | CGGCGCCGCCGGGGAACTGC | 237 |
| 8327 | GGCGCCGCCGGGGAACTGCG | 238 |
| 8328 | GCGCCGCCGGGGAACTGCGC | 239 |
| 8329 | CGCCGCCGGGGAACTGCGCC | 240 |
| 8330 | GCCGCCGGGGAACTGCGCCC | 241 |
| 8331 | CCGCCGGGGAACTGCGCCCG | 242 |
| 8332 | CGCCGGGGAACTGCGCCCGC | 243 |
| 8333 | GCCGGGGAACTGCGCCCGCT | 244 |
| 8334 | CCGGGGAACTGCGCCCGCTC | 245 |
| 8335 | CGGGGAACTGCGCCCGCTCG | 246 |
| 8336 | GGGGAACTGCGCCCGCTCGC | 247 |
| 8337 | GGGAACTGCGCCCGCTCGCG | 248 |
| 8338 | GGAACTGCGCCCGCTCGCGC | 249 |
| 8339 | GAACTGCGCCCGCTCGCGCC | 250 |
| 8340 | AACTGCGCCCGCTCGCGCCG | 251 |
| 8341 | ACTGCGCCCGCTCGCGCCGG | 252 |
| 8342 | CTGCGCCCGCTCGCGCCGGG | 253 |
| 8343 | TGCGCCCGCTCGCGCCGGGA | 254 |
| 8344 | GCGCCCGCTCGCGCCGGGAG | 255 |
| 8345 | CGCCCGCTCGCGCCGGGAGG | 256 |
| 8346 | GCCCGCTCGCGCCGGGAGGG | 257 |
| 8347 | CCCGCTCGCGCCGGGAGGGG | 258 |
| 8348 | CCGCTCGCGCCGGGAGGGGC | 259 |
| 8349 | CGCTCGCGCCGGGAGGGGCC | 260 |
| 8350 | GCTCGCGCCGGGAGGGGCCC | 261 |
| 8351 | CTCGCGCCGGGAGGGGCCCT | 262 |
| 8352 | TCGCGCCGGGAGGGGCCCTC | 263 |
| 8353 | CGCGCCGGGAGGGGCCCTCG | 264 |
| 8354 | GCGCCGGGAGGGGCCCTCGC | 265 |
| 8355 | CGCCGGGAGGGGCCCTCGCG | 266 |
| 8356 | GCCGGGAGGGGCCCTCGCGC | 267 |
| 8357 | CCGGGAGGGGCCCTCGCGCC | 268 |
| 8358 | CGGGAGGGGCCCTCGCGCCC | 269 |
| 8359 | GGGAGGGGCCCTCGCGCCCC | 270 |
| 8360 | GGAGGGGCCCTCGCGCCCCG | 271 |
| 8361 | GAGGGGCCCTCGCGCCCCGC | 272 |
| 8362 | AGGGGCCCTCGCGCCCCGCG | 273 |
| 8363 | GGGGCCCTCGCGCCCCGCGC | 274 |
| 8364 | GGGCCCTCGCGCCCCGCGCC | 275 |
| 8365 | GGCCCTCGCGCCCCGCGCCC | 276 |
| 8366 | GCCCTCGCGCCCCGCGCCCA | 277 |
| 8367 | CCCTCGCGCCCCGCGCCCAC | 278 |
| 8368 | CCTCGCGCCCCGCGCCCACA | 279 |
| 8369 | CTCGCGCCCCGCGCCCACAG | 280 |
| 8370 | TCGCGCCCCGCGCCCACAGG | 281 |
| 8371 | CGCGCCCCGCGCCCACAGGT | 282 |
| 8372 | GCGCCCCGCGCCCACAGGTG | 283 |
| 8373 | CGCCCCGCGCCCACAGGTGC | 284 |
| 8374 | GCCCCGCGCCCACAGGTGCA | 285 |
| 8375 | CCCCGCGCCCACAGGTGCAC | 286 |
| 8376 | CCCGCGCCCACAGGTGCACG | 287 |
| 8377 | CCGCGCCCACAGGTGCACGC | 288 |
| 8378 | CGCGCCCACAGGTGCACGCG | 289 |
| 8379 | GCGCCCACAGGTGCACGCGC | 290 |
| 8380 | CGCCCACAGGTGCACGCGCC | 291 |
| 8381 | GCCCACAGGTGCACGCGCCC | 292 |
| 8382 | CCCACAGGTGCACGCGCCCT | 293 |
| 8383 | CCACAGGTGCACGCGCCCTT | 294 |
| 8384 | CACAGGTGCACGCGCCCTTG | 295 |
| 8385 | ACAGGTGCACGCGCCCTTGG | 296 |
| 8386 | CAGGTGCACGCGCCCTTGGC | 297 |
| 8387 | AGGTGCACGCGCCCTTGGCG | 298 |
| 8388 | GGTGCACGCGCCCTTGGCGC | 299 |
| 8389 | GTGCACGCGCCCTTGGCGCC | 300 |
| 8390 | TGCACGCGCCCTTGGCGCCG | 301 |
| 8391 | GCACGCGCCCTTGGCGCCGC | 302 |
| 8392 | CACGCGCCCTTGGCGCCGCC | 303 |
| 8393 | ACGCGCCCTTGGCGCCGCCT | 304 |
| 8394 | CGCGCCCTTGGCGCCGCCTG | 305 |
| 8395 | GCGCCCTTGGCGCCGCCTGC | 306 |
| 8396 | CGCCCTTGGCGCCGCCTGCA | 307 |
| 8397 | GCCCTTGGCGCCGCCTGCAC | 308 |
| 8398 | CCCTTGGCGCCGCCTGCACC | 309 |
| 8399 | CCTTGGCGCCGCCTGCACCC | 310 |
| 8400 | CTTGGCGCCGCCTGCACCCC | 311 |
| 8401 | TTGGCGCCGCCTGCACCCCA | 312 |
| 8402 | TGGCGCCGCCTGCACCCCAC | 313 |
| 8403 | GGCGCCGCCTGCACCCCACG | 314 |
| 8404 | GCGCCGCCTGCACCCCACGC | 315 |
| 8405 | CGCCGCCTGCACCCCACGCG | 316 |
| 8406 | GCCGCCTGCACCCCACGCGC | 317 |
| 8407 | CCGCCTGCACCCCACGCGCC | 318 |
| 8408 | CGCCTGCACCCCACGCGCCC | 319 |
| 8409 | GCCTGCACCCCACGCGCCCC | 320 |
| 8410 | CCTGCACCCCACGCGCCCCC | 321 |
| 8411 | CTGCACCCCACGCGCCCCCT | 322 |
| 8412 | TGCACCCCACGCGCCCCCTC | 323 |
| 8413 | GCACCCCACGCGCCCCCTCC | 324 |
| 8414 | CACCCCACGCGCCCCCTCCG | 325 |
| 8415 | ACCCCACGCGCCCCCTCCGC | 326 |
| 8416 | CCCCACGCGCCCCCTCCGCT | 327 |
| 8417 | CCCACGCGCCCCCTCCGCTC | 328 |
| 8418 | CCACGCGCCCCCTCCGCTCC | 329 |
| 8419 | CACGCGCCCCCTCCGCTCCC | 330 |
| 8420 | ACGCGCCCCCTCCGCTCCCC | 331 |
| 8421 | CGCGCCCCCTCCGCTCCCCG | 332 |
| 8422 | GCGCCCCCTCCGCTCCCCGG | 333 |
| 8423 | CGCCCCCTCCGCTCCCCGGC | 334 |
| 8424 | GCCCCCTCCGCTCCCCGGCC | 335 |
| 8425 | CCGAGAGAGACCCCTAGCGG | 220 |
| 8426 | CCCGAGAGAGACCCCTAGCG | 219 |
| 8427 | ACCCGAGAGAGACCCCTAGC | 218 |
| 8428 | CACCCGAGAGAGACCCCTAG | 217 |
| 8429 | GCACCCGAGAGAGACCCCTA | 216 |
| 8430 | GGCACCCGAGAGAGACCCCT | 215 |
| 8431 | CGGCACCCGAGAGAGACCCC | 214 |
| 8432 | TCGGCACCCGAGAGAGACCC | 213 |
| 8433 | CTCGGCACCCGAGAGAGACC | 212 |
| 8434 | GCTCGGCACCCGAGAGAGAC | 211 |
| 8435 | CGCTCGGCACCCGAGAGAGA | 210 |
| 8436 | CCGCTCGGCACCCGAGAGAG | 209 |
| 8437 | CCCGCTCGGCACCCGAGAGA | 208 |
| 8438 | CCCCGCTCGGCACCCGAGAG | 207 |
| 8439 | ACCCCGCTCGGCACCCGAGA | 206 |
| 8440 | CACCCCGCTCGGCACCCGAG | 205 |
| 8441 | CCACCCCGCTCGGCACCCGA | 204 |
| 8442 | CCCACCCCGCTCGGCACCCG | 203 |
| 8443 | GCCCACCCCGCTCGGCACCC | 202 |
| 8444 | GGCCCACCCCGCTCGGCACC | 201 |
| 8445 | CGGCCCACCCCGCTCGGCAC | 200 |
| 8446 | CCGGCCCACCCCGCTCGGCA | 199 |
| 8447 | TCCGGCCCACCCCGCTCGGC | 198 |
| 8448 | ATCCGGCCCACCCCGCTCGG | 197 |
| 8449 | GATCCGGCCCACCCCGCTCG | 196 |
| 8450 | TGATCCGGCCCACCCCGCTC | 195 |
| 8451 | CTGATCCGGCCCACCCCGCT | 194 |
| 8452 | GCTGATCCGGCCCACCCCGC | 193 |
| 8453 | AGCTGATCCGGCCCACCCCG | 192 |
| 8454 | CAGCTGATCCGGCCCACCCC | 191 |
| 8455 | TCAGCTGATCCGGCCCACCC | 190 |
| 8456 | GTCAGCTGATCCGGCCCACC | 189 |
| 8457 | AGTCAGCTGATCCGGCCCAC | 188 |
| 8458 | GAGTCAGCTGATCCGGCCCA | 187 |
| 8459 | CGAGTCAGCTGATCCGGCCC | 186 |
| 8460 | GCGAGTCAGCTGATCCGGCC | 185 |
| 8461 | GGCGAGTCAGCTGATCCGGC | 184 |
| 8462 | AGGCGAGTCAGCTGATCCGG | 183 |
| 8463 | CAGGCGAGTCAGCTGATCCG | 182 |
| 8464 | CCAGGCGAGTCAGCTGATCC | 181 |
| 8465 | GCCAGGCGAGTCAGCTGATC | 180 |
| 8466 | AGCCAGGCGAGTCAGCTGAT | 179 |
| 8467 | GAGCCAGGCGAGTCAGCTGA | 178 |
| 8468 | AGAGCCAGGCGAGTCAGCTG | 177 |
| 8469 | CAGAGCCAGGCGAGTCAGCT | 176 |
| 8470 | TCAGAGCCAGGCGAGTCAGC | 175 |
| 8471 | CTCAGAGCCAGGCGAGTCAG | 174 |
| 8472 | GCTCAGAGCCAGGCGAGTCA | 173 |
| 8473 | GGCTCAGAGCCAGGCGAGTC | 172 |
| 8474 | GGGCTCAGAGCCAGGCGAGT | 171 |
| 8475 | CGCCCGCTCGCGCCGGGAGGGGCCCTCG | 256 |
| 8476 | GCCCGCTCGCGCCGGGAGGG | 257 |
| 8477 | CCCGCTCGCGCCGGGAGGGG | 258 |
| 8478 | CCGCTCGCGCCGGGAGGGGC | 259 |
| 8479 | CGCTCGCGCCGGGAGGGGCC | 260 |
| 8480 | GCTCGCGCCGGGAGGGGCCC | 261 |
| 8481 | CTCGCGCCGGGAGGGGCCCT | 262 |
| 8482 | TCGCGCCGGGAGGGGCCCTC | 263 |
| 8483 | CGCGCCGGGAGGGGCCCTCG | 264 |
| 8484 | GCGCCGGGAGGGGCCCTCGC | 265 |
| 8485 | CGCCGGGAGGGGCCCTCGCG | 266 |
| 8486 | GCCGGGAGGGGCCCTCGCGC | 267 |
| 8487 | CCGGGAGGGGCCCTCGCGCC | 268 |
| 8488 | CGGGAGGGGCCCTCGCGCCC | 269 |
| 8489 | GGGAGGGGCCCTCGCGCCCC | 270 |
| 8490 | GGAGGGGCCCTCGCGCCCCG | 271 |
| 8491 | GAGGGGCCCTCGCGCCCCGC | 272 |
| 8492 | AGGGGCCCTCGCGCCCCGCG | 273 |
| 8493 | GGGGCCCTCGCGCCCCGCGC | 274 |
| 8494 | GGGCCCTCGCGCCCCGCGCC | 275 |
| 8495 | GGCCCTCGCGCCCCGCGCCC | 276 |
| 8496 | GCCCTCGCGCCCCGCGCCCA | 277 |
| 8497 | CCCTCGCGCCCCGCGCCCAC | 278 |
| 8498 | CCTCGCGCCCCGCGCCCACA | 279 |
| 8499 | CTCGCGCCCCGCGCCCACAG | 280 |
| 8500 | TCGCGCCCCGCGCCCACAGG | 281 |
| 8501 | CGCGCCCCGCGCCCACAGGT | 282 |
| 8502 | GCGCCCCGCGCCCACAGGTG | 283 |
| 8503 | CGCCCCGCGCCCACAGGTGC | 284 |
| 8504 | GCCCCGCGCCCACAGGTGCA | 285 |
| 8505 | CCCCGCGCCCACAGGTGCAC | 286 |
| 8506 | CCCGCGCCCACAGGTGCACG | 287 |
| 8507 | CCGCGCCCACAGGTGCACGC | 288 |
| 8508 | CGCGCCCACAGGTGCACGCG | 289 |
| 8509 | GCGCCCACAGGTGCACGCGC | 290 |
| 8510 | CGCCCACAGGTGCACGCGCC | 291 |
| 8511 | GCCCACAGGTGCACGCGCCC | 292 |
| 8512 | CCCACAGGTGCACGCGCCCT | 293 |
| 8513 | CCACAGGTGCACGCGCCCTT | 294 |
| 8514 | CACAGGTGCACGCGCCCTTG | 295 |
| 8515 | ACAGGTGCACGCGCCCTTGG | 296 |
| 8516 | CAGGTGCACGCGCCCTTGGC | 297 |
| 8517 | AGGTGCACGCGCCCTTGGCG | 298 |
| 8518 | GGTGCACGCGCCCTTGGCGC | 299 |
| 8519 | GTGCACGCGCCCTTGGCGCC | 300 |
| 8520 | TGCACGCGCCCTTGGCGCCG | 301 |
| 8521 | GCACGCGCCCTTGGCGCCGC | 302 |
| 8522 | CACGCGCCCTTGGCGCCGCC | 303 |
| 8523 | ACGCGCCCTTGGCGCCGCCT | 304 |
| 8524 | CGCGCCCTTGGCGCCGCCTG | 305 |
| 8525 | GCGCCCTTGGCGCCGCCTGC | 306 |
| 8526 | CGCCCTTGGCGCCGCCTGCA | 307 |
| 8527 | GCCCTTGGCGCCGCCTGCAC | 308 |
| 8528 | CCCTTGGCGCCGCCTGCACC | 309 |
| 8529 | CCTTGGCGCCGCCTGCACCC | 310 |
| 8530 | CTTGGCGCCGCCTGCACCCC | 311 |
| 8531 | TTGGCGCCGCCTGCACCCCA | 312 |
| 8532 | TGGCGCCGCCTGCACCCCAC | 313 |
| 8533 | GGCGCCGCCTGCACCCCACG | 314 |
| 8534 | GCGCCGCCTGCACCCCACGC | 315 |
| 8535 | CGCCGCCTGCACCCCACGCG | 316 |
| 8536 | GCCGCCTGCACCCCACGCGC | 317 |
| 8537 | CCGCCTGCACCCCACGCGCC | 318 |
| 8538 | CGCCTGCACCCCACGCGCCC | 319 |
| 8539 | GCCTGCACCCCACGCGCCCC | 320 |
| 8540 | CCTGCACCCCACGCGCCCCC | 321 |
| 8541 | CTGCACCCCACGCGCCCCCT | 322 |
| 8542 | TGCACCCCACGCGCCCCCTC | 323 |
| 8543 | GCACCCCACGCGCCCCCTCC | 324 |
| 8544 | CACCCCACGCGCCCCCTCCG | 325 |
| 8545 | ACCCCACGCGCCCCCTCCGC | 326 |
| 8546 | CCCCACGCGCCCCCTCCGCT | 327 |
| 8547 | CCCACGCGCCCCCTCCGCTC | 328 |
| 8548 | CCACGCGCCCCCTCCGCTCC | 329 |
| 8549 | CACGCGCCCCCTCCGCTCCC | 330 |
| 8550 | ACGCGCCCCCTCCGCTCCCC | 331 |
| 8551 | CGCGCCCCCTCCGCTCCCCG | 332 |
| 8552 | GCGCCCCCTCCGCTCCCCGG | 333 |
| 8553 | CGCCCCCTCCGCTCCCCGGC | 334 |
| 8554 | GCCCCCTCCGCTCCCCGGCC | 335 |
| 8555 | GCGCCCGCTCGCGCCGGGAG | 255 |
| 8556 | TGCGCCCGCTCGCGCCGGGA | 254 |
| 8557 | CTGCGCCCGCTCGCGCCGGG | 253 |
| 8558 | ACTGCGCCCGCTCGCGCCGG | 252 |
| 8559 | AACTGCGCCCGCTCGCGCCG | 251 |
| 8560 | GAACTGCGCCCGCTCGCGCC | 250 |
| 8561 | GGAACTGCGCCCGCTCGCGC | 249 |
| 8562 | GGGAACTGCGCCCGCTCGCG | 248 |
| 8563 | GGGGAACTGCGCCCGCTCGC | 247 |
| 8564 | CGGGGAACTGCGCCCGCTCG | 246 |
| 8565 | CCGGGGAACTGCGCCCGCTC | 245 |
| 8566 | GCCGGGGAACTGCGCCCGCT | 244 |
| 8567 | CGCCGGGGAACTGCGCCCGC | 243 |
| 8568 | CCGCCGGGGAACTGCGCCCG | 242 |
| 8569 | GCCGCCGGGGAACTGCGCCC | 241 |
| 8570 | CGCCGCCGGGGAACTGCGCC | 240 |
| 8571 | GCGCCGCCGGGGAACTGCGC | 239 |
| 8572 | GGCGCCGCCGGGGAACTGCG | 238 |
| 8573 | CGGCGCCGCCGGGGAACTGC | 237 |
| 8574 | GCGGCGCCGCCGGGGAACTG | 236 |
| 8575 | AGCGGCGCCGCCGGGGAACT | 235 |
| 8576 | TAGCGGCGCCGCCGGGGAAC | 234 |
| 8577 | CTAGCGGCGCCGCCGGGGAA | 233 |
| 8578 | CCTAGCGGCGCCGCCGGGGA | 232 |
| 8579 | CCCTAGCGGCGCCGCCGGGG | 231 |
| 8580 | CCCCTAGCGGCGCCGCCGGG | 230 |
| 8581 | ACCCCTAGCGGCGCCGCCGG | 229 |
| 8582 | GACCCCTAGCGGCGCCGCCG | 228 |
| 8583 | AGACCCCTAGCGGCGCCGCC | 227 |
| 8584 | GAGACCCCTAGCGGCGCCGC | 226 |
| 8585 | AGAGACCCCTAGCGGCGCCG | 225 |
| 8586 | GAGAGACCCCTAGCGGCGCC | 224 |
| 8587 | AGAGAGACCCCTAGCGGCGC | 223 |
| 8588 | GAGAGAGACCCCTAGCGGCG | 222 |
| 8589 | CGAGAGAGACCCCTAGCGGC | 221 |
| 8590 | CCGAGAGAGACCCCTAGCGG | 220 |
| 8591 | CCCGAGAGAGACCCCTAGCG | 219 |
| 8592 | ACCCGAGAGAGACCCCTAGC | 218 |
| 8593 | CACCCGAGAGAGACCCCTAG | 217 |
| 8594 | GCACCCGAGAGAGACCCCTA | 216 |
| 8595 | GGCACCCGAGAGAGACCCCT | 215 |
| 8596 | CGGCACCCGAGAGAGACCCC | 214 |
| 8597 | TCGGCACCCGAGAGAGACCC | 213 |
| 8598 | CTCGGCACCCGAGAGAGACC | 212 |
| 8599 | GCTCGGCACCCGAGAGAGAC | 211 |
| 8600 | CGCTCGGCACCCGAGAGAGA | 210 |
| 8601 | CCGCTCGGCACCCGAGAGAG | 209 |
| 8602 | CCCGCTCGGCACCCGAGAGA | 208 |
| 8603 | CCCCGCTCGGCACCCGAGAG | 207 |
| 8604 | ACCCCGCTCGGCACCCGAGA | 206 |
| 8605 | CACCCCGCTCGGCACCCGAG | 205 |
| 8606 | CCACCCCGCTCGGCACCCGA | 204 |
| 8607 | CCCACCCCGCTCGGCACCCG | 203 |
| 8608 | GCCCACCCCGCTCGGCACCC | 202 |
| 8609 | GGCCCACCCCGCTCGGCACC | 201 |
| 8610 | CGGCCCACCCCGCTCGGCAC | 200 |
| 8611 | CCGGCCCACCCCGCTCGGCA | 199 |
| 8612 | TCCGGCCCACCCCGCTCGGC | 198 |
| 8613 | ATCCGGCCCACCCCGCTCGG | 197 |
| 8614 | GATCCGGCCCACCCCGCTCG | 196 |
| 8615 | TGATCCGGCCCACCCCGCTC | 195 |
| 8616 | CTGATCCGGCCCACCCCGCT | 194 |
| 8617 | GCTGATCCGGCCCACCCCGC | 193 |
| 8618 | AGCTGATCCGGCCCACCCCG | 192 |
| 8619 | CAGCTGATCCGGCCCACCCC | 191 |
| 8620 | TCAGCTGATCCGGCCCACCC | 190 |
| 8621 | GTCAGCTGATCCGGCCCACC | 189 |
| 8622 | AGTCAGCTGATCCGGCCCAC | 188 |
| 8623 | GAGTCAGCTGATCCGGCCCA | 187 |
| 8624 | CGAGTCAGCTGATCCGGCCC | 186 |
| 8625 | GCGAGTCAGCTGATCCGGCC | 185 |
| 8626 | GGCGAGTCAGCTGATCCGGC | 184 |
| 8627 | AGGCGAGTCAGCTGATCCGG | 183 |
| 8628 | CAGGCGAGTCAGCTGATCCG | 182 |
| 8629 | CCAGGCGAGTCAGCTGATCC | 181 |
| 8630 | GCCAGGCGAGTCAGCTGATC | 180 |
| 8631 | AGCCAGGCGAGTCAGCTGAT | 179 |
| 8632 | GAGCCAGGCGAGTCAGCTGA | 178 |
| 8633 | AGAGCCAGGCGAGTCAGCTG | 177 |
| 8634 | CAGAGCCAGGCGAGTCAGCT | 176 |
| 8635 | TCAGAGCCAGGCGAGTCAGC | 175 |
| 8636 | CTCAGAGCCAGGCGAGTCAG | 174 |
| 8637 | GCTCAGAGCCAGGCGAGTCA | 173 |
| 8638 | GGCTCAGAGCCAGGCGAGTC | 172 |
| 8639 | GGGCTCAGAGCCAGGCGAGT | 171 |
| 8640 | CGCGCCCACAGGTGCACGCGCCCTTGGCG | 289 |
| 8641 | GCGCCCACAGGTGCACGCGC | 290 |
| 8642 | CGCCCACAGGTGCACGCGCC | 291 |
| 8643 | GCCCACAGGTGCACGCGCCC | 292 |
| 8644 | CCCACAGGTGCACGCGCCCT | 293 |
| 8645 | CCACAGGTGCACGCGCCCTT | 294 |
| 8646 | CACAGGTGCACGCGCCCTTG | 295 |
| 8647 | ACAGGTGCACGCGCCCTTGG | 296 |
| 8648 | CAGGTGCACGCGCCCTTGGC | 297 |
| 8649 | AGGTGCACGCGCCCTTGGCG | 298 |
| 8650 | GGTGCACGCGCCCTTGGCGC | 299 |
| 8651 | GTGCACGCGCCCTTGGCGCC | 300 |
| 8652 | TGCACGCGCCCTTGGCGCCG | 301 |
| 8653 | GCACGCGCCCTTGGCGCCGC | 302 |
| 8654 | CACGCGCCCTTGGCGCCGCC | 303 |
| 8655 | ACGCGCCCTTGGCGCCGCCT | 304 |
| 8656 | CGCGCCCTTGGCGCCGCCTG | 305 |
| 8657 | GCGCCCTTGGCGCCGCCTGC | 306 |
| 8658 | CGCCCTTGGCGCCGCCTGCA | 307 |
| 8659 | GCCCTTGGCGCCGCCTGCAC | 308 |
| 8660 | CCCTTGGCGCCGCCTGCACC | 309 |
| 8661 | CCTTGGCGCCGCCTGCACCC | 310 |
| 8662 | CTTGGCGCCGCCTGCACCCC | 311 |
| 8663 | TTGGCGCCGCCTGCACCCCA | 312 |
| 8664 | TGGCGCCGCCTGCACCCCAC | 313 |
| 8665 | GGCGCCGCCTGCACCCCACG | 314 |
| 8666 | GCGCCGCCTGCACCCCACGC | 315 |
| 8667 | CGCCGCCTGCACCCCACGCG | 316 |
| 8668 | GCCGCCTGCACCCCACGCGC | 317 |
| 8669 | CCGCCTGCACCCCACGCGCC | 318 |
| 8670 | CGCCTGCACCCCACGCGCCC | 319 |
| 8671 | GCCTGCACCCCACGCGCCCC | 320 |
| 8672 | CCTGCACCCCACGCGCCCCC | 321 |
| 8673 | CTGCACCCCACGCGCCCCCT | 322 |
| 8674 | TGCACCCCACGCGCCCCCTC | 323 |
| 8675 | GCACCCCACGCGCCCCCTCC | 324 |
| 8676 | CACCCCACGCGCCCCCTCCG | 325 |
| 8677 | ACCCCACGCGCCCCCTCCGC | 326 |
| 8678 | CCCCACGCGCCCCCTCCGCT | 327 |
| 8679 | CCCACGCGCCCCCTCCGCTC | 328 |
| 8680 | CCACGCGCCCCCTCCGCTCC | 329 |
| 8681 | CACGCGCCCCCTCCGCTCCC | 330 |
| 8682 | ACGCGCCCCCTCCGCTCCCC | 331 |
| 8683 | CGCGCCCCCTCCGCTCCCCG | 332 |
| 8684 | GCGCCCCCTCCGCTCCCCGG | 333 |
| 8685 | CGCCCCCTCCGCTCCCCGGC | 334 |
| 8686 | GCCCCCTCCGCTCCCCGGCC | 335 |
| 8687 | CCGCGCCCACAGGTGCACGC | 288 |
| 8688 | CCCGCGCCCACAGGTGCACG | 287 |
| 8689 | CCCCGCGCCCACAGGTGCAC | 286 |
| 8690 | GCCCCGCGCCCACAGGTGCA | 285 |
| 8691 | CGCCCCGCGCCCACAGGTGC | 284 |
| 8692 | GCGCCCCGCGCCCACAGGTG | 283 |
| 8693 | CGCGCCCCGCGCCCACAGGT | 282 |
| 8694 | TCGCGCCCCGCGCCCACAGG | 281 |
| 8695 | CTCGCGCCCCGCGCCCACAG | 280 |
| 8696 | CCTCGCGCCCCGCGCCCACA | 279 |
| 8697 | CCCTCGCGCCCCGCGCCCAC | 278 |
| 8698 | GCCCTCGCGCCCCGCGCCCA | 277 |
| 8699 | GGCCCTCGCGCCCCGCGCCC | 276 |
| 8700 | GGGCCCTCGCGCCCCGCGCC | 275 |
| 8701 | GGGGCCCTCGCGCCCCGCGC | 274 |
| 8702 | AGGGGCCCTCGCGCCCCGCG | 273 |
| 8703 | GAGGGGCCCTCGCGCCCCGC | 272 |
| 8704 | GGAGGGGCCCTCGCGCCCCG | 271 |
| 8705 | GGGAGGGGCCCTCGCGCCCC | 270 |
| 8706 | CGGGAGGGGCCCTCGCGCCC | 269 |
| 8707 | CCGGGAGGGGCCCTCGCGCC | 268 |
| 8708 | GCCGGGAGGGGCCCTCGCGC | 267 |
| 8709 | CGCCGGGAGGGGCCCTCGCG | 266 |
| 8710 | GCGCCGGGAGGGGCCCTCGC | 265 |
| 8711 | CGCGCCGGGAGGGGCCCTCG | 264 |
| 8712 | TCGCGCCGGGAGGGGCCCTC | 263 |
| 8713 | CTCGCGCCGGGAGGGGCCCT | 262 |
| 8714 | GCTCGCGCCGGGAGGGGCCC | 261 |
| 8715 | CGCTCGCGCCGGGAGGGGCC | 260 |
| 8716 | CCGCTCGCGCCGGGAGGGGC | 259 |
| 8717 | CCCGCTCGCGCCGGGAGGGG | 258 |
| 8718 | GCCCGCTCGCGCCGGGAGGG | 257 |
| 8719 | CGCCCGCTCGCGCCGGGAGG | 256 |
| 8720 | GCGCCCGCTCGCGCCGGGAG | 255 |
| 8721 | TGCGCCCGCTCGCGCCGGGA | 254 |
| 8722 | CTGCGCCCGCTCGCGCCGGG | 253 |
| 8723 | ACTGCGCCCGCTCGCGCCGG | 252 |
| 8724 | AACTGCGCCCGCTCGCGCCG | 251 |
| 8725 | GAACTGCGCCCGCTCGCGCC | 250 |
| 8726 | GGAACTGCGCCCGCTCGCGC | 249 |
| 8727 | GGGAACTGCGCCCGCTCGCG | 248 |
| 8728 | GGGGAACTGCGCCCGCTCGC | 247 |
| 8729 | CGGGGAACTGCGCCCGCTCG | 246 |
| 8730 | CCGGGGAACTGCGCCCGCTC | 245 |
| 8731 | GCCGGGGAACTGCGCCCGCT | 244 |
| 8732 | CGCCGGGGAACTGCGCCCGC | 243 |
| 8733 | CCGCCGGGGAACTGCGCCCG | 242 |
| 8734 | GCCGCCGGGGAACTGCGCCC | 241 |
| 8735 | CGCCGCCGGGGAACTGCGCC | 240 |
| 8736 | GCGCCGCCGGGGAACTGCGC | 239 |
| 8737 | GGCGCCGCCGGGGAACTGCG | 238 |
| 8738 | CGGCGCCGCCGGGGAACTGC | 237 |
| 8739 | GCGGCGCCGCCGGGGAACTG | 236 |
| 8740 | AGCGGCGCCGCCGGGGAACT | 235 |
| 8741 | TAGCGGCGCCGCCGGGGAAC | 234 |
| 8742 | CTAGCGGCGCCGCCGGGGAA | 233 |
| 8743 | CCTAGCGGCGCCGCCGGGGA | 232 |
| 8744 | CCCTAGCGGCGCCGCCGGGG | 231 |
| 8745 | CCCCTAGCGGCGCCGCCGGG | 230 |
| 8746 | ACCCCTAGCGGCGCCGCCGG | 229 |
| 8747 | GACCCCTAGCGGCGCCGCCG | 228 |
| 8748 | AGACCCCTAGCGGCGCCGCC | 227 |
| 8749 | GAGACCCCTAGCGGCGCCGC | 226 |
| 8750 | AGAGACCCCTAGCGGCGCCG | 225 |
| 8751 | GAGAGACCCCTAGCGGCGCC | 224 |
| 8752 | AGAGAGACCCCTAGCGGCGC | 223 |
| 8753 | GAGAGAGACCCCTAGCGGCG | 222 |
| 8754 | CGAGAGAGACCCCTAGCGGC | 221 |
| 8755 | CCGAGAGAGACCCCTAGCGG | 220 |
| 8756 | CCCGAGAGAGACCCCTAGCG | 219 |
| 8757 | ACCCGAGAGAGACCCCTAGC | 218 |
| 8758 | CACCCGAGAGAGACCCCTAG | 217 |
| 8759 | GCACCCGAGAGAGACCCCTA | 216 |
| 8760 | GGCACCCGAGAGAGACCCCT | 215 |
| 8761 | CGGCACCCGAGAGAGACCCC | 214 |
| 8762 | TCGGCACCCGAGAGAGACCC | 213 |
| 8763 | CTCGGCACCCGAGAGAGACC | 212 |
| 8764 | GCTCGGCACCCGAGAGAGAC | 211 |
| 8765 | CGCTCGGCACCCGAGAGAGA | 210 |
| 8766 | CCGCTCGGCACCCGAGAGAG | 209 |
| 8767 | CCCGCTCGGCACCCGAGAGA | 208 |
| 8768 | CCCCGCTCGGCACCCGAGAG | 207 |
| 8769 | ACCCCGCTCGGCACCCGAGA | 206 |
| 8770 | CACCCCGCTCGGCACCCGAG | 205 |
| 8771 | CCACCCCGCTCGGCACCCGA | 204 |
| 8772 | CCCACCCCGCTCGGCACCCG | 203 |
| 8773 | GCCCACCCCGCTCGGCACCC | 202 |
| 8774 | GGCCCACCCCGCTCGGCACC | 201 |
| 8775 | CGGCCCACCCCGCTCGGCAC | 200 |
| 8776 | CCGGCCCACCCCGCTCGGCA | 199 |
| 8777 | TCCGGCCCACCCCGCTCGGC | 198 |
| 8778 | ATCCGGCCCACCCCGCTCGG | 197 |
| 8779 | GATCCGGCCCACCCCGCTCG | 196 |
| 8780 | TGATCCGGCCCACCCCGCTC | 195 |
| 8781 | CTGATCCGGCCCACCCCGCT | 194 |
| 8782 | GCTGATCCGGCCCACCCCGC | 193 |
| 8783 | AGCTGATCCGGCCCACCCCG | 192 |
| 8784 | CAGCTGATCCGGCCCACCCC | 191 |
| 8785 | TCAGCTGATCCGGCCCACCC | 190 |
| 8786 | GTCAGCTGATCCGGCCCACC | 189 |
| 8787 | AGTCAGCTGATCCGGCCCAC | 188 |
| 8788 | GAGTCAGCTGATCCGGCCCA | 187 |
| 8789 | CGAGTCAGCTGATCCGGCCC | 186 |
| 8790 | GCGAGTCAGCTGATCCGGCC | 185 |
| 8791 | GGCGAGTCAGCTGATCCGGC | 184 |
| 8792 | AGGCGAGTCAGCTGATCCGG | 183 |
| 8793 | CAGGCGAGTCAGCTGATCCG | 182 |
| 8794 | CCAGGCGAGTCAGCTGATCC | 181 |
| 8795 | GCCAGGCGAGTCAGCTGATC | 180 |
| 8796 | AGCCAGGCGAGTCAGCTGAT | 179 |
| 8797 | GAGCCAGGCGAGTCAGCTGA | 178 |
| 8798 | AGAGCCAGGCGAGTCAGCTG | 177 |
| 8799 | CAGAGCCAGGCGAGTCAGCT | 176 |
| 8800 | TCAGAGCCAGGCGAGTCAGC | 175 |
| 8801 | CTCAGAGCCAGGCGAGTCAG | 174 |
| 8802 | GCTCAGAGCCAGGCGAGTCA | 173 |
| 8803 | GGCTCAGAGCCAGGCGAGTC | 172 |
| 8804 | GGGCTCAGAGCCAGGCGAGT | 171 |
| 8805 | GGCCGACGGCCCACCTGGGCTTCG | 351 |
| 8806 | GCCGACGGCCCACCTGGGCT | 352 |
| 8807 | CCGACGGCCCACCTGGGCTT | 353 |
| 8808 | CGACGGCCCACCTGGGCTTC | 354 |
| 8809 | GACGGCCCACCTGGGCTTCG | 355 |
| 8810 | ACGGCCCACCTGGGCTTCGT | 356 |
| 8811 | CGGCCCACCTGGGCTTCGTG | 357 |
| 8812 | GGCCCACCTGGGCTTCGTGA | 358 |
| 8813 | GCCCACCTGGGCTTCGTGAA | 359 |
| 8814 | CCCACCTGGGCTTCGTGAAC | 360 |
| 8815 | CCACCTGGGCTTCGTGAACA | 361 |
| 8816 | CACCTGGGCTTCGTGAACAG | 362 |
| 8817 | ACCTGGGCTTCGTGAACAGT | 363 |
| 8818 | CCTGGGCTTCGTGAACAGTG | 364 |
| 8819 | CTGGGCTTCGTGAACAGTGG | 365 |
| 8820 | TGGGCTTCGTGAACAGTGGG | 366 |
| 8821 | GGGCTTCGTGAACAGTGGGA | 367 |
| 8822 | GGCTTCGTGAACAGTGGGAG | 368 |
| 8823 | GCTTCGTGAACAGTGGGAGG | 369 |
| 8824 | CTTCGTGAACAGTGGGAGGG | 370 |
| 8825 | CGCTGAGGCTCTAGAAAAGTCGAGAG | 446 |
| 8826 | ACGCTGAGGCTCTAGAAAAG | 445 |
| 8827 | GACGCTGAGGCTCTAGAAAA | 444 |
| 8828 | GGACGCTGAGGCTCTAGAAA | 443 |
| 8829 | AGGACGCTGAGGCTCTAGAA | 442 |
| 8830 | TAGGACGCTGAGGCTCTAGA | 441 |
| 8831 | CTAGGACGCTGAGGCTCTAG | 440 |
| 8832 | CCTAGGACGCTGAGGCTCTA | 439 |
| 8833 | TCCTAGGACGCTGAGGCTCT | 438 |
| 8834 | GTCCTAGGACGCTGAGGCTC | 437 |
| 8835 | AGTCCTAGGACGCTGAGGCT | 436 |
| 8836 | GAGTCCTAGGACGCTGAGGC | 435 |
| 8837 | TGAGTCCTAGGACGCTGAGG | 434 |
| 8838 | GTGAGTCCTAGGACGCTGAG | 433 |
| 8839 | GGTGAGTCCTAGGACGCTGA | 432 |
| 8840 | AGGTGAGTCCTAGGACGCTG | 431 |
| 8841 | AAGGTGAGTCCTAGGACGCT | 430 |
| 8842 | AAAGGTGAGTCCTAGGACGC | 429 |
| 8843 | CTCGTCCCCGTGAGCTTGAATCATCCGACCC | 480 |
| 8844 | TCGTCCCCGTGAGCTTGAAT | 481 |
| 8845 | CGTCCCCGTGAGCTTGAATC | 482 |
| 8846 | GTCCCCGTGAGCTTGAATCA | 483 |
| 8847 | TCCCCGTGAGCTTGAATCAT | 484 |
| 8848 | CCCCGTGAGCTTGAATCATC | 485 |
| 8849 | CCCGTGAGCTTGAATCATCC | 486 |
| 8850 | CCGTGAGCTTGAATCATCCG | 487 |
| 8851 | CGTGAGCTTGAATCATCCGA | 488 |
| 8852 | GTGAGCTTGAATCATCCGAC | 489 |
| 8853 | TGAGCTTGAATCATCCGACC | 490 |
| 8854 | GAGCTTGAATCATCCGACCC | 491 |
| 8855 | AGCTTGAATCATCCGACCCC | 492 |
| 8856 | GCTTGAATCATCCGACCCCG | 493 |
| 8857 | CTTGAATCATCCGACCCCGC | 494 |
| 8858 | TTGAATCATCCGACCCCGCA | 495 |
| 8859 | TGAATCATCCGACCCCGCAG | 496 |
| 8860 | GAATCATCCGACCCCGCAGG | 497 |
| 8861 | AATCATCCGACCCCGCAGGC | 498 |
| 8862 | ATCATCCGACCCCGCAGGCC | 499 |
| 8863 | TCATCCGACCCCGCAGGCCT | 500 |
| 8864 | CATCCGACCCCGCAGGCCTC | 501 |
| 8865 | ATCCGACCCCGCAGGCCTCC | 502 |
| 8866 | TCCGACCCCGCAGGCCTCCC | 503 |
| 8867 | CCGACCCCGCAGGCCTCCCG | 504 |
| 8868 | CGACCCCGCAGGCCTCCCGG | 505 |
| 8869 | GACCCCGCAGGCCTCCCGGG | 506 |
| 8870 | ACCCCGCAGGCCTCCCGGGG | 507 |
| 8871 | CCCCGCAGGCCTCCCGGGGG | 508 |
| 8872 | CCCGCAGGCCTCCCGGGGGT | 509 |
| 8873 | CCGCAGGCCTCCCGGGGGTG | 510 |
| 8874 | CGCAGGCCTCCCGGGGGTGT | 511 |
| 8875 | GCAGGCCTCCCGGGGGTGTC | 512 |
| 8876 | CAGGCCTCCCGGGGGTGTCG | 513 |
| 8877 | AGGCCTCCCGGGGGTGTCGT | 514 |
| 8878 | GGCCTCCCGGGGGTGTCGTA | 515 |
| 8879 | GCCTCCCGGGGGTGTCGTAT | 516 |
| 8880 | CCTCCCGGGGGTGTCGTATA | 517 |
| 8881 | CTCCCGGGGGTGTCGTATAA | 518 |
| 8882 | TCCCGGGGGTGTCGTATAAA | 519 |
| 8883 | CCCGGGGGTGTCGTATAAAG | 520 |
| 8884 | CCGGGGGTGTCGTATAAAGG | 521 |
| 8885 | GCTCGTCCCCGTGAGCTTGA | 479 |
| 8886 | TGCTCGTCCCCGTGAGCTTG | 478 |
| 8887 | CTGCTCGTCCCCGTGAGCTT | 477 |
| 8888 | CCTGCTCGTCCCCGTGAGCT | 476 |
| 8889 | TCCTGCTCGTCCCCGTGAGC | 475 |
| 8890 | CTCCTGCTCGTCCCCGTGAG | 474 |
| 8891 | GCTCCTGCTCGTCCCCGTGA | 473 |
| 8892 | CGCTCCTGCTCGTCCCCGTG | 472 |
| 8893 | GCGCTCCTGCTCGTCCCCGT | 471 |
| 8894 | AGCGCTCCTGCTCGTCCCCG | 470 |
| 8895 | GAGCGCTCCTGCTCGTCCCC | 469 |
| 8896 | AGAGCGCTCCTGCTCGTCCC | 468 |
| 8897 | GAGAGCGCTCCTGCTCGTCC | 467 |
| 8898 | CGAGAGCGCTCCTGCTCGTC | 466 |
| 8899 | TCGAGAGCGCTCCTGCTCGT | 465 |
| 8900 | GTCGAGAGCGCTCCTGCTCG | 464 |
| 8901 | AGTCGAGAGCGCTCCTGCTC | 463 |
| 8902 | AAGTCGAGAGCGCTCCTGCT | 462 |
| 8903 | AAAGTCGAGAGCGCTCCTGC | 461 |
| 8904 | AAAAGTCGAGAGCGCTCCTG | 460 |
| 8905 | GAAAAGTCGAGAGCGCTCCT | 459 |
| 8906 | AGAAAAGTCGAGAGCGCTCC | 458 |
| 8907 | TAGAAAAGTCGAGAGCGCTC | 457 |
| 8908 | CTAGAAAAGTCGAGAGCGCT | 456 |
| 8909 | TCTAGAAAAGTCGAGAGCGC | 455 |
| 8910 | CTCTAGAAAAGTCGAGAGCG | 454 |
| 8911 | GCTCTAGAAAAGTCGAGAGC | 453 |
| 8912 | AGGCGTTTCTGGAAGAGAATGAGAACG | 604 |
| 8913 | GGCGTTTCTGGAAGAGAATG | 605 |
| 8914 | GCGTTTCTGGAAGAGAATGA | 606 |
| 8915 | CGTTTCTGGAAGAGAATGAG | 607 |
| 8916 | CAGGCGTTTCTGGAAGAGAA | 603 |
| 8917 | GCAGGCGTTTCTGGAAGAGA | 602 |
| 8918 | GGCAGGCGTTTCTGGAAGAG | 601 |
| 8919 | GGGCAGGCGTTTCTGGAAGA | 600 |
| 8920 | GGGGCAGGCGTTTCTGGAAG | 599 |
| 8921 | TGGGGCAGGCGTTTCTGGAA | 598 |
| 8922 | GTGGGGCAGGCGTTTCTGGA | 597 |
| 8923 | GGTGGGGCAGGCGTTTCTGG | 596 |
| 8924 | AGGTGGGGCAGGCGTTTCTG | 595 |
| 8925 | GAGGTGGGGCAGGCGTTTCT | 594 |
| 8926 | AGAGGTGGGGCAGGCGTTTC | 593 |
| 8927 | CGTCAAAAGCAGGCACGAGCAACCTG | 701 |
| 8928 | GAACGAACCAAAGGAGCAAGGCG | 742 |
| 8929 | CGCTGACAAGGGTGCCTAGGCCCGG | 1318 |
| 8930 | GCGCTGACAAGGGTGCCTAG | 1317 |
| 8931 | TGCGCTGACAAGGGTGCCTA | 1316 |
| 8932 | TTGCGCTGACAAGGGTGCCT | 1315 |
| 8933 | ATTGCGCTGACAAGGGTGCC | 1314 |
| 8934 | CATTGCGCTGACAAGGGTGC | 1313 |
| 8935 | TCATTGCGCTGACAAGGGTG | 1312 |
| 8936 | CTCATTGCGCTGACAAGGGT | 1311 |
| 8937 | GCTCATTGCGCTGACAAGGG | 1310 |
| 8938 | TGCTCATTGCGCTGACAAGG | 1309 |
| 8939 | TTGCTCATTGCGCTGACAAG | 1308 |
| 8940 | CTTGCTCATTGCGCTGACAA | 1307 |
| 8941 | CCTTGCTCATTGCGCTGACA | 1306 |
| 8942 | CCCTTGCTCATTGCGCTGAC | 1305 |
| 8943 | TCCCTTGCTCATTGCGCTGA | 1304 |
| 8944 | CTCCCTTGCTCATTGCGCTG | 1303 |
| 8945 | TCTCCCTTGCTCATTGCGCT | 1302 |
| 8946 | CTCTCCCTTGCTCATTGCGC | 1301 |
| 8947 | TCTCTCCCTTGCTCATTGCG | 1300 |
| 8948 | CGCAATTCCGTATTTGTTCCGG | 1738 |
| 8949 | GCAATTCCGTATTTGTTCCG | 1739 |
| 8950 | CAATTCCGTATTTGTTCCGG | 1740 |
| 8951 | AATTCCGTATTTGTTCCGGG | 1741 |
| 8952 | ATTCCGTATTTGTTCCGGGT | 1742 |
| 8953 | TTCCGTATTTGTTCCGGGTC | 1743 |
| 8954 | TCCGTATTTGTTCCGGGTCT | 1744 |
| 8955 | CCGTATTTGTTCCGGGTCTG | 1745 |
| 8956 | CGTATTTGTTCCGGGTCTGC | 1746 |
| 8957 | GTATTTGTTCCGGGTCTGCA | 1747 |
| 8958 | TATTTGTTCCGGGTCTGCAT | 1748 |
| 8959 | ATTTGTTCCGGGTCTGCATG | 1749 |
| 8960 | TTTGTTCCGGGTCTGCATGA | 1750 |
| 8961 | TTGTTCCGGGTCTGCATGAG | 1751 |
| 8962 | TGTTCCGGGTCTGCATGAGC | 1752 |
| 8963 | GTTCCGGGTCTGCATGAGCA | 1753 |
| 8964 | TTCCGGGTCTGCATGAGCAA | 1754 |
| 8965 | TCCGGGTCTGCATGAGCAAA | 1755 |
| 8966 | CCGGGTCTGCATGAGCAAAT | 1756 |
| 8967 | CGGGTCTGCATGAGCAAATA | 1757 |
| 8968 | CCGCAATTCCGTATTTGTTC | 1737 |
| 8969 | GTACGTTGGCAGACGCAGTGACG | 4923 |
| 8970 | TACGTTGGCAGACGCAGTGA | 4924 |
| 8971 | ACGTTGGCAGACGCAGTGAC | 4925 |
| 8972 | CGTTGGCAGACGCAGTGACG | 4926 |
| 8973 | GTTGGCAGACGCAGTGACGT | 4927 |
| 8974 | TTGGCAGACGCAGTGACGTA | 4928 |
| 8975 | TGGCAGACGCAGTGACGTAT | 4929 |
| 8976 | GGCAGACGCAGTGACGTATT | 4930 |
| 8977 | GCAGACGCAGTGACGTATTT | 4931 |
| 8978 | CAGACGCAGTGACGTATTTG | 4932 |
| 8979 | AGACGCAGTGACGTATTTGA | 4933 |
| 8980 | GACGCAGTGACGTATTTGAG | 4934 |
| 8981 | ACGCAGTGACGTATTTGAGA | 4935 |
| 8982 | CGCAGTGACGTATTTGAGAG | 4936 |
| 8983 | GCAGTGACGTATTTGAGAGT | 4937 |
| 8984 | TGTACGTTGGCAGACGCAGT | 4922 |
| 8985 | ATGTACGTTGGCAGACGCAG | 4921 |
| 8986 | CATGTACGTTGGCAGACGCA | 4920 |
| 8987 | TCATGTACGTTGGCAGACGC | 4919 |
| 8988 | ATCATGTACGTTGGCAGACG | 4918 |
| 8989 | TATCATGTACGTTGGCAGAC | 4917 |
| 8990 | GTATCATGTACGTTGGCAGA | 4916 |
| 8991 | GGTATCATGTACGTTGGCAG | 4915 |
| 8992 | GGGTATCATGTACGTTGGCA | 4914 |
| 8993 | TGGGTATCATGTACGTTGGC | 4913 |
| 8994 | CTGGGTATCATGTACGTTGG | 4912 |
| 8995 | GCTGGGTATCATGTACGTTG | 4911 |
| 8996 | TGCTGGGTATCATGTACGTT | 4910 |
| 8997 | TTGCTGGGTATCATGTACGT | 4909 |
| 8998 | CTTGCTGGGTATCATGTACG | 4908 |

Hot Zones (Relative upstream location to gene start site)
1-800
1200-1800
4800-5100

### Examples

Genetic Code (5′ Upstream Region)

(SEQ ID NO: 11978)

TGAAGACTATAGCCCCTTTCTTTTGGCTGATTTCTCCCTTTTGGAATGGG
AATGTTTACCCAGTGCTGGTACTCCCATTATATCTTGGAAGTAAATAACT
TGTTTTGATTTTACAGGCTCATAGATGGAAAGAGATGAGTCTCAGATGAG
ACTTTGGACTTGGAATTTGGACTTTGGACTTTTGAGTTAATGCTGGAACA
AGTCAAGACCTTGGAGGACTGTTGGGAAGGCATGATTGTATTTTGAAATG
AGAGAGGGACATGAGATTTGAGAGGGGCTGGGGCAGAATGATATAGTTTG
TGTACTTATCCCCACCCAAATCTCATGTGGAATTGTAATCCCTAGTATTG
GAGGCGGGACCTGGTAGGAGGTGATTGGATCATGGGGGTGGATTACTCAT
GAATCGTTTAGCACTATCTGTTTGTTGCTGTCCTTGTGATGAGTGACTTC
TCATGAGATCTGGCTGTTTAAAAGTGTGTGGCACCATGCTTTCTCTTGTT
CCCGGTCTGGTCATGTGACATCCCTACTCCCCCTTCACCTTCCATCATGA
TTGTAAGTTTCCTGAGGCCTCACCAGAAGCCAAGCAGATGCCAGCATCAG
GCTTCCTGCAAAGCCTGAAGAATCATAAGCTGATTAAATCTCTCTCTCTT
TTTTTTTGAGATGGAGTCTCATCCTGTGGCCCACGCTGGAAGGCAGTGGC
ACTATCTCTGCTCTGAGGCTCAAGTGATTCTCCTGCCTCAGCCTCCTGAG
TAGCTAGGATTACAGGTGCATGCCACCACACCCAGCTAATTTTTGTATTT
TTAGTAGAGATGAGGTTTCACCACGTTGGCCAGGCTGGTCTCGAACTCCT
GACCTAAGGTTATCTGTCCACCTCAGCTTCCCAAAGTGCTGGGATTACAG
GTGTGAGCCATTGCACCCGGCCTCTTTTCTTTATAAACTACCTAGTCTCA
GATATTTCTTTTTGGCAATGCAAGAATGGCCTAATACACCAAGATTTGTG
TTTTCACCATTTTTTTTCTGTTTATAACCATGTTATTTTACTGTTTATCT
GAGAAAAGAACCTGGCCTGTTAGTATTTTTTATCGACTGACAAACTCACC
AGAATAAAGTGGGTATTAGGACCCTTGGTTCTGCAAGATTTTGGTAACAG
AATCTGTTTTCACTAATTGTTGGGAAACAGAAATGATATTCCTTTTAGAC
CTAGCTCCCTAAACCTTTTCCTCGTTTTGCTTTTTGGTACAATAATGAGG
GCTGGCAGGGCTACTTGACACCATTAGCAGTAGACAAATTTTTCAATAAG
GACTAACAGAGAAAAACTATGGAAATTCTGATTTATTGTTTGGCCGAGAG
AGTTCTCTGTCTCCTTGTGCCCTTGCTCTCCATGTATATTTTATGAGACA
TTTCAGCAAAGGCATCTCTCAAAATTAATCCCATAGCTGTCTCCTTCAAT
TCTCCTCCTTGAAACTCTTCACCAATCTTCATAGGGCCTTGGCCACTTCT
TGAGGGCCCTCCCACCAGATGATTAAAGGCAATACAGTGAACGAAAGTCT
TATTCCGAGACTTGTCTTTGTAAACTTAGTGATCCTTCTTCCTTTTCACT
TACGAAAATTTAAAGAGAAGCAGTCTCAAATGTGAACTGAATGCCGTCCC
ATTACTCCCCCAAACTGGGAAGAAGCTGGTCATATACTTGCACATTTATA
TAATAAATATTCAAAGACTCTAATCTGGTATCTTCCATATAACACACACT
TTAACTCCTATTTTAAACTTTCAAAAGGCTTTTTATGGCATCTTATGCCC
TACTTTAAAATGTCTGTCAGCCTAATATTTCTACTTTTTTTTTATTAATT
TATTTTCAAGGTCATGTGTGAAAACAACTTTCAGTGAAAAGAACCCATCT
GCTTTGACAAAAATGTACACTATAAACCTTCACTTCTACAAGGGTCTAAA
AAAATTCAAGGGTTTGATTCGAATGCTTCCAAACCACATTCCCTAAAGCA
TGCTTTGTGCAACACTAGGAGTTGGTGCAGCAAACAAAAACAAGGAATGG
GGAGAGGTGCCAGGCAGGCGCGGTGGCTCATGCCTGTAATCCCAGCACTT
TGGGTGACCCAGGCAGGTGGATCACTTGAGGTCAGGAGTTCGAGATCAGT
CAACATGGTGAAACCCCGTCTCTTCTAAAATACAAAAATTAGCTAGGTGT
GTGTGCACGTAATCCCAGCACTTTGTGAGGCTGAGGTGGGTGGATCACTT
GAGGTCAGGAGTTCAAGACCAGCCTGGCCAACATGGTGAAACCCCATCTC
TACTAAAAATACAAAAAAAAAAATTAGCCGGGTATGGTGGCACACACCTG
TAATCCCAGCCACTCAGGAGGCTGAGGCATGAGAATTGCTTGATCCCTGG
AGGCAGAGGTTGCAGTGAGCAGAGATCATGCCTGGGGAAAAGAGAGAGAC
TATGTCTCAAAAAAAAAAAAAAGAAAAAAGAAATAGGGAGAGGGAGTGAT
GCTACATACTGAAGCTACCTACCCTCTTTAAAATTCAAAATGCAGATTAT
CATCTTAAAGAATTGTATGCATTTTAAAGTGAAGGTATTTGTTTAATTTT
GTTTAACCTCTTATTTCTCAAAGTTACTTGATTACAGAATTCTTTTCGGG
TATGCATGCAATACCGAGCAACATATGACCGAGCTAGGGTTCCATGGTAC
ACAGTTTGGAACTTGTGCTAGAAAATGTTACTCATTATTTTATAAGAAGT
ATTATTCCAAGGCATCTATGCATTGGAAAATAATCTTGATATGAAATATG
ACTCGATCCCTTCTCATACCATATTTACAGGGTATGGTGGTTAACTTTAT
GTGTCCACTTGTCTCTTAGCCCATCCTGGGTTAAGGGATGCCCAGACAGC
TGGTAAAATATTATTTCTGGGAGAGTCTGTGAGAGTATTTCTGGAAGAGA
TTAGCATTTGAATCAATAGACTGGGTATCAAAAACCCATCCTCACTAATG
TGAGTGGGCATCATTCAATTAACTGAGCATAGGAATCATAAAAGAGGTGG
AGGAAGGCAAATTTGCTCTCTCTTCTGAAGCTGGGACATCCATCATCTGC
TCCTGCCCTTGACTTCAGAGCTCCAGGTTCTTGAGCCTTCAATACCAGCA
GACACTCTTCAGCCCTTCAGCTTCACATGCAAGAATTACACTGGATTTCC
TGGTTCTTCGGCTTGCAGGGGGCACATATTGGAACTTCTTGGCCTTTATA
ATCTCCTGAGCCAATTCCTATAAGAAGTGTCTTTATATATCTATATATAT
CCCATTGGTTCTGTTTGTTTGGAAAACCCTGATACACAGGGCTATCTATA
GCTCACCCCCCAAGTACTAAGTCTCCAGATGATTATTAGGTTCTTATAAA
CACAAAATATATATGCTTATTTTGTAATATCAGGTTGTTAACTTCATCTG
AGTAGTTTTCAGCACATATGATGGGGATAAGTCATCAGTTATGACAGACA
ACTTCCGGAGTTACATCACCAATGTTCATCTATCACTCACCTTTGCTTCA
AACTTTATGCATCATATTCAAGTTAATTCCTATTGCATGTACTTCTGATT
TCCACCTACAAAATGAACATTAAATTTATTATTTCCCTCATTTAAAAAAA
AAGATTTCAGGCCAGGTGCAGTGGCTCATGCCTGCAATCACAGCACTTTG
GGAGGCTGAGGCGGGTGGATCACTTGAGGCCAGGAGTTCAAGACCAGCCT
AGGCAACATGATGAAACCCCTATCTCTACTAAAAATACAAAATACAAAAA
AAAAAAAAATTAGCCAGGTGTATGTGTGCCTGCAATCCCACCCCTACTCA
GGAGGCTGAGGCAGGAGGATAGCTTGAGTTCTGGAGGCGGAGGTTGCCGT
GAGCCAAGATCACGCCACTGTACTCAAGCCTGGGCAAGACAGCAAGACTG
AGACTCTGTCTCAAAAAAAAAAAAAAAAAAAGTTTTCAAATCACTTTTTC
TCCTTTGCAGTTCAGCATTGTCACTAAAGGTGTCCAGGTTAGACAGAACT
GGGTACAAACCCTCCTTTCTCTTCCCCTTACTCTTTCACCTCTTCATTTG
TGACATGCAATGTTAACCCAATACAACTCATGAGTATTCAGAGATCCCGT
CTGTATACGTCCTCAGCCTGACATACTGTAATCCTTAGGCATCCTTATTA
GTAATAAGATGTCCTTGTGTGATTTTTTCACAAACTTTTCACAGACCCTA
TCTATGTTCATTCCTGGAACCTCTGGCACATTCTTCTTCCTTCTCTTCCC
AATCTCAACTTTTTCATCCTCTGAATCTCCCTATACTTTCCCCGTGGACA
AGCCTCTAGAAATGTTAAAATGTCAGATCATGATTGGTAAATCTGTAGTG
ACTAATTGCCCACTGCTGCCTATTCCATCTGACCTAAATTCCTCAGGTCT
TCTAACATTAAGACCTCTTCCTGGCCGGTTGTGCTGGCTCATGCCTGTAA
TTCCAACACTTTAGGCAGCTGAGGCAGGCAGATCACTTGAGGTCAAGAGT
TCAAGACCAGCCTGGCCAACATGGTGAAGCCCTGTCTGTACTAAAAAATA
CAAAAATTAGCCGGACCTGGTGGTGCGTGCCTGTAATCCCAGCTACTCGG
GAGGCTGAGTCAGGAGAATCACTTGAACCCGGGGCAGCGGGGGAGGCTGC
AGTGAGTGGAGATCAAACCACCGCACTCCAGCCCAGGTGACAGAGCAAGA
GTCAGTCTCAAAAAAAAAAAAACAAAAAAAAAAACCTCTTCCTATAGCTA
ACTCCCACTTACCACCCCCATCATGAACACTCTTGATGTATTTACATGGT
TTCTCCTTCGAACATCCTCCTTTCTTCTTTCTTAATGGTTGTTATCAAAT
ACCCTGATAAAAAACAAAAACAAAAAACCTCCTCTGAAGGTCCCTTATTC
ACCCTTCCAACGCTACAGGTCTGTAACTCTCATTTTCTTTTTAAAAAATT
TTTATTTTTTTAATTTATTTTATTTTTTTTTTCAGACGGAGTCTTGCTCT
GTCGCCCAGGCTGGAGTGCAGTGACACGATCTCGGCTCACTGCAACCTCC
ACTTCCCAGGTTCAAGCAATTCTCCTGCCTCAGCCTCCTGAGTTGCTGGG
ATTACAGGTGCCTACCACCACACCTGACCTCAAGTAACCCACCCACCTCG
ACCTCCCAAAGTGCTGAGAATACAGGTGTGAGCCATCATGCCTGGCCAAA
ATTTTTAAATTTTAAAAAATATATTTTATTTTTTGTAGAGACAGGGTCTC
ATTTTGAGCCCAAACTGGTCTTGAACTCCTAGGCTCAAGTGATCCTCCTG
CCTTGGCCTCCCAAAATGCTGGGATTATAGGCACAAGCCACCAGGCCTGA
TCCTTACTTTTCTTCTGATGAATTCACATATATGTGCACAAATACTTTAT
ACTAAATTGTATTTACTGATGTACTTTTTTCACTGTGCCTTTTCTTTTTC
TTGCCCAGATATTTTTCTCATATAAACATTAGCTCCTTAATGGGAGCAAA
TGAACCAGTTTTTTTTTAATTCCCACCCAAAGTGAGAATATAAAAATTTT
TTATTGATCCACCAATACTGAACACTTTCATTTCTAATAGTTATATTTAA
CTGAATAAATTACACACGGGACAAAAATGTTATTTAAGGGATAAAGTTGG
GTGTTTGCTCAGGGACAACGTTGTATATTGAATGATTTGGTGCTTTTGTG
AATTTATCATTCAAAAGACCATCGTGATGGCTAAATAACAGAAAGGAGAG
CTTTATTGGCAATATCAATTTGCAAACCCGGAAGACATAGTCTTCGGTGT
ATGCTGAATGTGGTCTCTCTTCAAAAGAGAGGAAGGACAGTTGGGTTTCA
TGCCTCACAGGGTCTGTTTCACACAGTGGAGTCATACATATTCAGCAGGT
TTGGAGGAAAAGATATACATATTTATGAGGGGAGCTGAGTGCATGTGCAA
TGGGTAAATATGTATGTGACATCCCATGTACACTTTGGGGCAGGGTTTTA
GTGTTAAAATGAGGTAAAATTTGGCTCTTTACATCAAAAGGTGAACTACA
GGACCCAAAGACAGTTTGTGCACAGCCTCTAATAAACTGGCTGACACTGG
CTTAAGGTCTGCAATTGCTTATCAGAAAAGAATGTTTGTAAGGCTGGTCC
TCATTCCAATTAGAGTTGTAGTGGTCTGGGTTGTAAATCACAGGATGGGG
CTGATAGTTCCTATTATTAGGGAGTTTAGAGCCATAGAAATTGAGAAATT
GGTCATGCCAGCCAGTCCCCGAACCCTAACCCTGTAGGTAACTTTGTTTC
CTTAACCTTACAGTCCATCTTAGGTGATAAAGGGGTGTCTGTTTTGGTAT
CTCACATCACAAATTGTTGGTTGGTTTGTGTGTTTGTTTCATCATTCAGG
ATGTTGTTTCTTTAGGGAATGTGAACCTGAATTCTCAAGGCTTGTTAGAC
TGTAATGTTCCCATTCATTTTAGGTTTAGCTCATGCTTCTCTAGCCACAG
CCTTCACTTGGATTTTAAAAGTTGAATTACTCATCAAAGTCTCTAGGACA
CGAAAGACAATCCTTAGGTATGATTTGACCAGTAAAAAAGAGATCCAGCT
GCCTTGAAGCATAAGATCCCCTCGGCTCCAATGTCTATCACTAATATTCA
GTGTGGCAAGGATCCCAGGCCACAGAGCTGTGGCTTCCTGCAGCTGCTCT
GGGGAGTGACTCTCTTGGAGCATGTGATGTGGTCTTCCATTGTGCAGGAC
CAGCCCAGTGGCATCCTTTCAACACCTCTGGCAAGCAGCCTTTCCAAGCA
CGGGTGCCGTCTGAAAACAGGAGGCATATCTTTCACATCCTAGGCACACG
CCCTAGGGAGTGGTCAGGGTTTTGTCCAGTTCTCAGCAAACTAGCTACAG
CTCCATCCCTTACTCCCACACTCAAGAGAGATACTAGAATACAACTGAGA
GTAGCCTGATATGATGCTAACCTCGAGTTGCTTTTATTTAAATTAAAATA
AATCAACCAGACACAGTGGCTCATGCCTATAATCCCTGCATTTTAGGAGA
TCAAGGAGGGTGGATCACGAGCTCAGGAGTTTAAGACCAACCTGGATAAC
ATGGCAAGACCCCATCTCTACAAAAAGTACACAAATTAGCTGGACATGGT
AGTGCGCACCTGTAGCCCCAGCTACTCTGGAGGCTGAGGTGAAGGATCAC
TTGAGCCCAGGAGGTAGAGGTTGCAGTGAGCTGAGATTGTGCCACTGCTA
ATAATTAATTAAATAATTAATTAAATTAATAAATCGTGCCACTTTATTAA
ATAAATAAAACAAGAGTAAATCACTCACAAATTTGGAGCTTTTATTAGCA
AAACATTACTTAGGAAATCTAAATAAATAACACGGGGTTGACAGCCATTG
TTCTAACTGGCAGCCCCTGGCAAGCTCAAAGCCAGGATTATGCTGGTCAC
TTAAGTGACAGCTATTGCGAATTGTTGTTCTCTCAAGAAAAAAGAACCGA
TTTCTATGGTAAACCAGGCACTGTGCTGGGTGCCTTTACAATTCATCACC
ACACCACCTAATGAAAGGAGCATTCTTCAGAAACTGTAGTGCTCAGGCTT
TCTCAAGGCCTGAGTTCTTTTCCACCAGAGCATATTGTTGCCCTATTATC
CAAAGTTCTCTAAGGAAGAGAACTGACGTAAGACCCACATGGCTCCATTA
CATCTTCTGGCTACTTGATTGATTTTCATACTCCCTACCTCTGGGGTTGG
TATGTACTATCTATTTCTTTCTCCTCTCGTTCTTCCTTTTTATTCCATAA
AATACAGGAATATTCCTGTACATTAGTCCTTGCAGCAACCTTGGAATTAC
TACATTCCTCAAACAAGTTATGGAAGCCAGCTGCCAATATTGGTCCCTGG
TTAAACAGTGAATTCTGTTGTTCCATAGAGTTACTACTGAAATACCTAAG
CCATTTTGTAAAATATAATTTAGTTGATCTGAAGGCTGTCTCTAAAGCAG
TTTTATGTAGTGATTACAGAGAAGGACTAATTTCAAGAGTATTTTATTGT
TTAAAAAAATGTAAACATTTTATGGATGCACTAGTGAAGTAAAGACCAAT
AAATGAAGCAGTAACTTTAATAAAAGGGTAAGTAAAATGTCACATCCTCT
GCCTATATTCAGGTCTGTTAGGTATGTGTAGTTAAATGTAGGTAAGTTAG
TTGATAATTATTTATTTAAGCATTTCTTTATGTCTACTCATTAAAAAGAA
AAAAAGATTAAAAGAATGTTACTATGTGAAAAACTGCCCATCACTGGGGA
AAAGAATTTTATTATGCAAAGCTTCAACGCTATTTACAGTTTAGACTTTT
GTAGCTATTGAAGGCTGACATTGAGATAAAGAAGTTAATCATGTCCTTCT
GTCTTGGAGGAGGTAGAAAGAGATGAGAATGAATACAATTCAGGATCTAC
TTCTGGTCTTTGATGAGGAGTTAGCACACGGTTCTGGGAGGAAAGACAGG
TTAAGAGGCATGTGAAACTCTCAAATACGTCACTGCGTCTGCCAACGTAC
ATGATACCCAGCAAGCTCACATCTTCATGGAAAGCATGGTAATTCCCAAC
ACTACCGGAAGTCTGGAGTGGCTAAGTAATCCATATATTCAACCAGGAAG
CAGCTAAAGAAATATTCTAATTACCTAGGAAGGTTTCTGATTTCAAAAGG
ACATGAATAAAAAGTAGAAGGAATCCACTCCCAAGGACGGACATCAGAGT
AGCTTAAAATGTGAGAATAATTTTAGGGGAATTTTAGAGGTTTGGTTATA
GACTTATGTTCCCCCAAAATTCATATGTTGAAGCCCTAACCCCCAGTACC
TTAGAACATGACTGTATTTGGGTAGGGCCTTTGAAGAGCTAATTAAATTA
AGGCCACTGGCGTGGGCCCTAATATAATCTGGCTGGTATTCTTGTAAGAG
GAGGAGATTAGGACACACAGAAATACCAGAGGTACCTGTGCAGAGGAAAG
AACGTGTGAGGACTTAGCAAGGGTGCAGCCATCTGCAAGCCAAGGAGACC
TCTGAGGATTCCAATCCTATCTGCATCTTGATCTTAGACTTTTCTGGAAC
TGTGAGAAAATAAATTTCTTGGTTTAAGCCACCCAGTCTGTGATATTTTG
TTATGGCAGCTCTAGTAAACTAATACAGATTTTAAATGTCATTAAATGTC
AATGTTTAAGCTTTGACAAAATTTTCTAAAGGAAAGTATAAAAGGTCATT
TTCTTTCTTTTCAGAGCCTGATGATTGCGGGAGGGGTAAGCCAGCTGCAT
GGGGATCATGATGCAATGCTGATGCAGGACAGACAGAAAGTAGATCTCTT
CCATTTCTATTTTTTTTTTTTCTGTTGAGTTGAATGATCTTCAGACTGAA
AATGAAAGAAAGGTCACTGGAAATAAAGGCCAAAGATGAGTGACAGGATT
ATAGAATAAGTCTTAGCTGTTCTAAAGAAGGACATATTATGTACCCCCAC
CCCCAAATTCATATGTTGAAGTCCTAACCCGACAGTGTCTCAAAATGTGA
CCATATTTGGAGATAGGGTCAAAGATGTAATTAAGGTTAAATGAGGTCAT
TAGCATGGATCCTAACCCAATATCTGCTGTCCTTATAACAAGAGGAGATT
AGGGCACAGTAAGACACAGAGGGAAGACCATGTGAGAATACAGGGAGAAG
GTGGCCATCTGCAAGCCAAGGAGAGAGGCCTCAGAAGTAACCAACTCAGC
CAACACCTCGATTTCAGACTTCCAGCCTCCTGAAATGTGAGGAAATACAT
TTCTGGTGTTTGATCCATCCAGTCTATGGTAAGTTATGGCACCCCTGCAG
GGTTCATCTGGCTCAGACTTAACGATTGCTTTTGGTGATATTTATAGGGC
ACAGATAACAGCCTAAACACAAGACGACAGAAACGCGGCCCAGCAGACTA
TGCATAAAATAGAAATGGGGTATCTGGACCAATTGGAGTCTGCAGTGGGA
TGCGGTTACTAAAACAGTCAAATGCAACATGAGGCTCCAGGCAGAGTAGT
GGGCAACATCTCCCATGTTGCAGCAGTCAGAGCACACTTCGAGTACTGTA
AAAAGACACAGACAAGCCAGAACACATTTAGAGAATGGCCAAGGTGTGGA
AGGAACCAGAAACCATGCCATTATGCAACTGTTGAAGGAAGTGCCTGTTT
TACCTTGTGAAGAGAAGACTCTAGAGGAAGAAGTAGCATGAAAACCGCTG
GCAAATTTGTAAAGATCTGAAGTGTGGAAAAGAATTATTCTGCTTGGTCA
CTGGGGATACAAGGATATCTGAGTGGGAGTTTAAAGGCGGGGGATGTGAG
CTTTAAATGGGATAAGAACATTCTAGTAACCAGAAATGCCCAAAGATAGA
ATGCACAGTCTGGAGAGCCAGTGAATATCTCACAAATGGAGACACTTGAA
ACTAGGATGGGGATGCTGTTGTAGGAATTCCAGCAGACAAGTGGTTGTTG
GTTCCTTCCCCAACTTTGTAGGGTTATAACTAGGGATGTTCCTGCGTTTT
CTGCTTGGAGGATCTGCAAGACACCTCAGGGCAGGAAATGGCATTAAATG
CAGAACAGAGCTAGTGGCTGAAAAGCAAAAAGCCATCAGGATCTCTGAGT
AGTGAAGGAACCAGAGAACATGCAGGCAATGTCCATCATTCTGACGCAAT
CAGCAGCATAATCATCTTCCCCCAGGAACATCTTGACCAGGGAATGTGTC
AGTGTGGTGAATTTCAACAGTGGAAAGAGAAACTGCTAAATCTAAGAACT
TTAATTTTTATAGATTATGATCTCATCTCTACAATTTTGAATTTCATGCT
CAATAAAAGTTCCTTACTCTCTTTTTTTTTTTTTGAGACGGAGTCTCGCT
CTGTCGCCCAGGCTGGAGTGCAGTGGCGCGATCTCGGCTCACTTCAAGCT
CAGCCTCCCGGGTTCACGCCATTCTCCTGCCTCAGCCTCCCCAGTAGCTG
GGACTACAGGCGCCCGCCACGACGCCCGGCTAATTTTTTGTATTTTTAGT
AGAGACGGGGTTTCACCGTGTTAGCCAGGATGGTGTTGATCTCCTGACCT
CGTGATCCGCCCGCCTCAGCCTCCCAAAGAAAAGTCCCTCACTCTTAAAG
TTGCCTCCTCCTTCCCAGGGCTGGCTTCATGGGCATGCAACCCTGGAGAG
TCTCACAGGCCCTGCGGTGGGAGGAGCCCCATGCTTGGTTTAACGCTCTG
CCATTGCCATCTTAAAATTCTTAATTTAATTTTTTTTCTTTTTTTTGAGG
TGGAGTCTCGCTCTGTCGCCCAGGCTGGAGTGCAATGGCACAATCTTGGC
TCACTGCAACCTCCGCCTCCCAGGTTCAAGCGATTCTCCTGCCTCAGCCT
CTGGAGTAGCTGGGATTACAGGCAGGAGTAACCACGCTCGGCTAATTTTT
GCATTTTTAGTAGAGATGGGGGTTTCACCATGTTGGCCAGGCTGGTCTAG
AACTCCTGACCTCAGGTGATCTGCCCACCTGGGCCTCCTAAAGTGCTGGG
ATTACAGGCATGAGCCACCAGGCCCGGCCTTAAAATTCTTAATAATGTAA
CAAAGGGTCTCACGTTTGCATTTTGCAGTGGACTCTGCAAGATTTGTAGC
TTTGGACCACGTTTCTCTTTGCATTCAGATACCTTCTTTTTTGCCTTATT
TGCTCATGCAGACCCGGAACAAATACGGAATTGCGGTGGGTAAATGTGGT
GCAGAAAGTGAACAACTGGGTTTGTCCTGTCACTTTAGGCTTTTCCCTGC
TGTCCCAGCTTCATGTCACTTACTTGCTATTAGATTTGGGAGTTCATTAG
CTTCATTTTCCTGATGTATAAATAGGAATAATAGTAACAGCCTCTTTGGC
TTTTGTAGGAAGTAAATGACATGAAGCGTATAAACAAATACTGCATGACA
ATAAATATTTGTCCTTATTTGTTGAGGACATCCAAAGGACATTCAGGGGC
AAAAGTAATCCAAGAGTCAAGACTGAATGCCTAGTGCGGGAAAAGACACA
CAAGACAACATTTAGGGGAGCTGGTACAGAAATGACTTCCCAGGAAGGAA
GTCTGTACCCCGCTGGCTGAGCCATCCTTCCCGGGCCTAGGCACCCTTGT
CAGCGCAATGAGCAAGGGAGAGAAGGCAGGCTGCAGTGCAGCCCTCAGAA
GGGCCAGAGCACTCCCTGGCTTCAGTCCTTCGCTCCAAGCCCTGTGTGGA
GTGGGCTGTGGCTTGGTAACTAAATGCTACTTCAGGTCAAGAGCAGGGGA
TATATCTGGGCAGTTCTAGAGCATTCTAAACTATCTGGACACTAACTGGA
CAGTGGACGGTTTGTGTTTAATCCAGGAGAAAGTGGCATGGCAGAAGGTT
CATTTCTATAATTCAGGACAGACACAATGAAGAACAAGGGCAGCGTTTGA
GGTCAGAAGTCCTCATTTACGGGGGTCGAATACGAATGATCTCTCCTAAT
TTTTCCTTCTTCCCCAACTCAGATGGATGTTACATCCCTGCTTAACAACA
AAAAAAGACCCCCCGCCCCGCAAAATCCACACTGACCACCCCCTTTAACA
AAACAAAACCAAAAACAAACAAAAATATAAGAAAGAAACAAAACCCAAGC
CCAGAACCCTGCTTTCAAGAAGAAGTAAATGGGTTGGCCGCTTCTTTGCC
AGGTCCTGCGCCTTGCTCCTTTGGTTCGTTCTAAAGATAGAAATTCCAGG
TTGCTCGTGCCTGCTTTTGACGTTGGGGGTTAAAAAATGAGGTTTTGCTG
TCTCAACAAGCAAAGAAAATCCTATTTCCTTTAAGCTTCACTCGTTCTCA
TTCTCTTCCAGAAACGCCTGCCCCACCTCTCCAAACCGAGAGAAAAAACG
AAATGCGGATAAAAACGCACCCTAGCAGCAGTCCTTTATACGACACCCCC
GGGAGGCCTGCGGGGTCGGATGATTCAAGCTCACGGGGACGAGCAGGAGC
GCTCTCGACTTTTCTAGAGCCTCAGCGTCCTAGGACTCACCTTTCCCTGA
TCCTGCACCGTCCCTCTCCTGGCCCCAGACTCTCCCTCCCACTGTTCACG
AAGCCCAGGTGGGCCGTCGGCCGGGGAGCGGAGGGGGCGCGTGGGGTGCA
GGCGGCGCCAAGGGCGCGTGCACCTGTGGGCGCGGGGCGCGAGGGCCCCT
CCCGGCGCGAGCGGGCGCAGTTCCCCGGCGGCGCCGCTAGGGGTCTCTCT
CGGGTGCCGAGCGGGGTGGGCCGGATCAGCTGACTCGCCTGGCTCTGAGC
CCCGCCGCCGCGCTCGGGCTCCGTCAGTTTCCTCGGCAGCGGTAGGCGAG
AGCACGCGGAGGAGCGTGCGCGGGGGCCCCGGGAGACGGCGGCGGTGGCG
GCGCGGGCAGAGCAAGGACGCGGCGGATCCCACTCGCACAGCAGCGCACT
CGGTGCCCCGCGCAGGGTCGCGATG

32. HAMP

Hepcidin is a peptide hormone produced by the liver. Hepcidin plays a role in maintaining iron balance by inhibiting iron absorption across the gut mucosa and transport of iron from macrophages which serve as a depot of iron storage and transport. Hepcidin production in the liver increases when iron enters liver cells from the blood thereby causing its release into the blood. In contrast, in states of high hepcidin (e.g. inflammation), serum iron levels drop because iron remains trapped in macrophages, resulting in anemia (Ganz T. 2003. Blood 102 (3): 783-8). Beta-thalassemia a common congenital anemia is characterized by excessive iron absorption and overload of iron associated with low levels hepcidin levels. In this situation, increasing expression of hepcidin may be therapeutic to treat the abnormal iron absorption in individuals with β-thalassemia and related disorders. Mutations in this gene cause hemochromatosis type 2B, also known as juvenile hemochromatosis, a disease caused by severe iron overload resulting in cardiomyopathy, cirrhosis, and endocrine failure.

Protein: HAMP Gene: HAMP (Homo sapiens, chromosome 19, 35773410-35776064 [NCBI Reference Sequence: NC_{—}000019.9]; start site location: 35773482; strand: positive)

Gene Identification

| | |
|---|---|
| GeneID | 57817 |
| HGNC | 15598 |
| HPRD | 05925 |
| MIM | 606464 |

Targeted Sequences

| | | | Relative upstream |
|---|---|---|---|
| Sequence | Design | | location to gene start |
| ID No: | ID | Sequence (5′-3′) | site |
| 8999 | | CGTGCCGTCTGTCTGGCTGTCCCAC | 1 |
| 9005 | | CGAGTGACAGTCGCTTTTATGGGGC | 60 |
| 9035 | | CGGGGCATGGCCAGCAGCCGCCAGG | 424 |
| 9086 | | CGTGTGCCCGATCCGCACGTGGTGT | 563 |
| 9121 | | CGACAGGCTGACGGGCCAAGCTTGG | 2344 |
| 9150 | | CGGATGGGCAGGGAGGATACCGTTT | 3109 |
| 9151 | | CGTGGGCGGCGGCGGCTGCGTGGTG | 3287 |

Target Shift Sequences

| | | Relative |
|---|---|---|
| | | upstream |
| Sequence | | location to gene |
| ID No: | Sequence (5′-3′) | start site |
| 8999 | CGTGCCGTCTGTCTGGCTGTCCCAC | 1 |
| 9000 | GTGCCGTCTGTCTGGCTGTC | 2 |
| 9001 | TGCCGTCTGTCTGGCTGTCC | 3 |
| 9002 | GCCGTCTGTCTGGCTGTCCC | 4 |
| 9003 | CCGTCTGTCTGGCTGTCCCA | 5 |
| 9004 | CGTCTGTCTGGCTGTCCCAC | 6 |
| 9005 | CGAGTGACAGTCGCTTTTATGGGGC | 60 |
| 9006 | GAGTGACAGTCGCTTTTATG | 61 |
| 9007 | AGTGACAGTCGCTTTTATGG | 62 |
| 9008 | GTGACAGTCGCTTTTATGGG | 63 |
| 9009 | TGACAGTCGCTTTTATGGGG | 64 |
| 9010 | GACAGTCGCTTTTATGGGGC | 65 |
| 9011 | ACAGTCGCTTTTATGGGGCC | 66 |
| 9012 | CAGTCGCTTTTATGGGGCCT | 67 |
| 9013 | AGTCGCTTTTATGGGGCCTG | 68 |
| 9014 | GTCGCTTTTATGGGGCCTGC | 69 |
| 9015 | TCGCTTTTATGGGGCCTGCC | 70 |
| 9016 | CGCTTTTATGGGGCCTGCCA | 71 |
| 9017 | CCGAGTGACAGTCGCTTTTA | 59 |
| 9018 | ACCGAGTGACAGTCGCTTTT | 58 |
| 9019 | GACCGAGTGACAGTCGCTTT | 57 |
| 9020 | GGACCGAGTGACAGTCGCTT | 56 |
| 9021 | GGGACCGAGTGACAGTCGCT | 55 |
| 9022 | TGGGACCGAGTGACAGTCGC | 54 |
| 9023 | CTGGGACCGAGTGACAGTCG | 53 |
| 9024 | TCTGGGACCGAGTGACAGTC | 52 |
| 9025 | GTCTGGGACCGAGTGACAGT | 51 |
| 9026 | TGTCTGGGACCGAGTGACAG | 50 |
| 9027 | GTGTCTGGGACCGAGTGACA | 49 |
| 9028 | GGTGTCTGGGACCGAGTGAC | 48 |
| 9029 | TGGTGTCTGGGACCGAGTGA | 47 |
| 9030 | CTGGTGTCTGGGACCGAGTG | 46 |
| 9031 | TCTGGTGTCTGGGACCGAGT | 45 |
| 9032 | CTCTGGTGTCTGGGACCGAG | 44 |
| 9033 | GCTCTGGTGTCTGGGACCGA | 43 |
| 9034 | TGCTCTGGTGTCTGGGACCG | 42 |
| 9035 | CGGGGCATGGCCAGCAGCCGCCAGG | 424 |
| 9036 | GGGGCATGGCCAGCAGCCGC | 425 |
| 9037 | GGGCATGGCCAGCAGCCGCC | 426 |
| 9038 | GGCATGGCCAGCAGCCGCCA | 427 |
| 9039 | GCATGGCCAGCAGCCGCCAG | 428 |
| 9040 | CATGGCCAGCAGCCGCCAGG | 429 |
| 9041 | ATGGCCAGCAGCCGCCAGGC | 430 |
| 9042 | TGGCCAGCAGCCGCCAGGCT | 431 |
| 9043 | GGCCAGCAGCCGCCAGGCTC | 432 |
| 9044 | GCCAGCAGCCGCCAGGCTCC | 433 |
| 9045 | CCAGCAGCCGCCAGGCTCCT | 434 |
| 9046 | CAGCAGCCGCCAGGCTCCTC | 435 |
| 9047 | AGCAGCCGCCAGGCTCCTCA | 436 |
| 9048 | GCAGCCGCCAGGCTCCTCAG | 437 |
| 9049 | CAGCCGCCAGGCTCCTCAGG | 438 |
| 9050 | AGCCGCCAGGCTCCTCAGGA | 439 |
| 9051 | GCCGCCAGGCTCCTCAGGAG | 440 |
| 9052 | CCGCCAGGCTCCTCAGGAGT | 441 |
| 9053 | CGCCAGGCTCCTCAGGAGTG | 442 |
| 9054 | ACGGGGCATGGCCAGCAGCC | 423 |
| 9055 | CACGGGGCATGGCCAGCAGC | 422 |
| 9056 | ACACGGGGCATGGCCAGCAG | 421 |
| 9057 | CACACGGGGCATGGCCAGCA | 420 |
| 9058 | GCACACGGGGCATGGCCAGC | 419 |
| 9059 | TGCACACGGGGCATGGCCAG | 418 |
| 9060 | ATGCACACGGGGCATGGCCA | 417 |
| 9061 | CATGCACACGGGGCATGGCC | 416 |
| 9062 | ACATGCACACGGGGCATGGC | 415 |
| 9063 | TACATGCACACGGGGCATGG | 414 |
| 9064 | CTACATGCACACGGGGCATG | 413 |
| 9065 | CCTACATGCACACGGGGCAT | 412 |
| 9066 | GCCTACATGCACACGGGGCA | 411 |
| 9067 | CGCCTACATGCACACGGGGC | 410 |
| 9068 | TCGCCTACATGCACACGGGG | 409 |
| 9069 | ATCGCCTACATGCACACGGG | 408 |
| 9070 | CATCGCCTACATGCACACGG | 407 |
| 9071 | CCATCGCCTACATGCACACG | 406 |
| 9072 | CCCATCGCCTACATGCACAC | 405 |
| 9073 | CCCCATCGCCTACATGCACA | 404 |
| 9074 | TCCCCATCGCCTACATGCAC | 403 |
| 9075 | TTCCCCATCGCCTACATGCA | 402 |
| 9076 | CTTCCCCATCGCCTACATGC | 401 |
| 9077 | ACTTCCCCATCGCCTACATG | 400 |
| 9078 | CACTTCCCCATCGCCTACAT | 399 |
| 9079 | TCACTTCCCCATCGCCTACA | 398 |
| 9080 | CTCACTTCCCCATCGCCTAC | 397 |
| 9081 | ACTCACTTCCCCATCGCCTA | 396 |
| 9082 | CACTCACTTCCCCATCGCCT | 395 |
| 9083 | CCACTCACTTCCCCATCGCC | 394 |
| 9084 | TCCACTCACTTCCCCATCGC | 393 |
| 9085 | CTCCACTCACTTCCCCATCG | 392 |
| 9086 | CGTGTGCCCGATCCGCACGTGGTGT | 563 |
| 9087 | GTGTGCCCGATCCGCACGTG | 564 |
| 9088 | TGTGCCCGATCCGCACGTGG | 565 |
| 9089 | GTGCCCGATCCGCACGTGGT | 566 |
| 9090 | TGCCCGATCCGCACGTGGTG | 567 |
| 9091 | GCCCGATCCGCACGTGGTGT | 568 |
| 9092 | CCCGATCCGCACGTGGTGTT | 569 |
| 9093 | CCGATCCGCACGTGGTGTTT | 570 |
| 9094 | CGATCCGCACGTGGTGTTTT | 571 |
| 9095 | GATCCGCACGTGGTGTTTTC | 572 |
| 9096 | ATCCGCACGTGGTGTTTTCC | 573 |
| 9097 | TCCGCACGTGGTGTTTTCCC | 574 |
| 9098 | CCGCACGTGGTGTTTTCCCA | 575 |
| 9099 | CGCACGTGGTGTTTTCCCAG | 576 |
| 9100 | GCACGTGGTGTTTTCCCAGT | 577 |
| 9101 | CACGTGGTGTTTTCCCAGTG | 578 |
| 9102 | ACGTGGTGTTTTCCCAGTGT | 579 |
| 9103 | CGTGGTGTTTTCCCAGTGTC | 580 |
| 9104 | GCGTGTGCCCGATCCGCACG | 562 |
| 9105 | AGCGTGTGCCCGATCCGCAC | 561 |
| 9106 | CAGCGTGTGCCCGATCCGCA | 560 |
| 9107 | TCAGCGTGTGCCCGATCCGC | 559 |
| 9108 | ATCAGCGTGTGCCCGATCCG | 558 |
| 9109 | CATCAGCGTGTGCCCGATCC | 557 |
| 9110 | GCATCAGCGTGTGCCCGATC | 556 |
| 9111 | AGCATCAGCGTGTGCCCGAT | 555 |
| 9112 | AAGCATCAGCGTGTGCCCGA | 554 |
| 9113 | CAAGCATCAGCGTGTGCCCG | 553 |
| 9114 | GCAAGCATCAGCGTGTGCCC | 552 |
| 9115 | GGCAAGCATCAGCGTGTGCC | 551 |
| 9116 | GGGCAAGCATCAGCGTGTGC | 550 |
| 9117 | AGGGCAAGCATCAGCGTGTG | 549 |
| 9118 | CAGGGCAAGCATCAGCGTGT | 548 |
| 9119 | GCAGGGCAAGCATCAGCGTG | 547 |
| 9120 | AGCAGGGCAAGCATCAGCGT | 546 |
| 9121 | CGACAGGCTGACGGGCCAAGCTTGG | 2344 |
| 9122 | GACAGGCTGACGGGCCAAGC | 2345 |
| 9123 | ACAGGCTGACGGGCCAAGCT | 2346 |
| 9124 | CAGGCTGACGGGCCAAGCTT | 2347 |
| 9125 | AGGCTGACGGGCCAAGCTTG | 2348 |
| 9126 | GGCTGACGGGCCAAGCTTGG | 2349 |
| 9127 | GCTGACGGGCCAAGCTTGGC | 2350 |
| 9128 | CTGACGGGCCAAGCTTGGCG | 2351 |
| 9129 | TGACGGGCCAAGCTTGGCGC | 2352 |
| 9130 | GACGGGCCAAGCTTGGCGCC | 2353 |
| 9131 | ACGGGCCAAGCTTGGCGCCC | 2354 |
| 9132 | CGGGCCAAGCTTGGCGCCCT | 2355 |
| 9133 | GGGCCAAGCTTGGCGCCCTG | 2356 |
| 9134 | GGCCAAGCTTGGCGCCCTGG | 2357 |
| 9135 | GCCAAGCTTGGCGCCCTGGC | 2358 |
| 9136 | CCAAGCTTGGCGCCCTGGCC | 2359 |
| 9137 | CAAGCTTGGCGCCCTGGCCA | 2360 |
| 9138 | AAGCTTGGCGCCCTGGCCAT | 2361 |
| 9139 | AGCTTGGCGCCCTGGCCATC | 2362 |
| 9140 | GCTTGGCGCCCTGGCCATCT | 2363 |
| 9141 | CTTGGCGCCCTGGCCATCTG | 2364 |
| 9142 | TTGGCGCCCTGGCCATCTGC | 2365 |
| 9143 | TGGCGCCCTGGCCATCTGCC | 2366 |
| 9144 | GGCGCCCTGGCCATCTGCCC | 2367 |
| 9145 | GCGCCCTGGCCATCTGCCCT | 2368 |
| 9146 | CGCCCTGGCCATCTGCCCTC | 2369 |
| 9147 | GCGACAGGCTGACGGGCCAA | 2343 |
| 9148 | GGCGACAGGCTGACGGGCCA | 2342 |
| 9149 | AGGCGACAGGCTGACGGGCC | 2341 |
| 9150 | CGGATGGGCAGGGAGGATACCGTTT | 3109 |
| 9151 | CGTGGGCGGCGGCGGCTGCGTGGTG | 3287 |
| 9152 | GTGGGCGGCGGCGGCTGCGT | 3288 |
| 9153 | TGGGCGGCGGCGGCTGCGTG | 3289 |
| 9154 | GGGCGGCGGCGGCTGCGTGG | 3290 |
| 9155 | GGCGGCGGCGGCTGCGTGGT | 3291 |
| 9156 | GCGGCGGCGGCTGCGTGGTG | 3292 |
| 9157 | CGGCGGCGGCTGCGTGGTGG | 3293 |
| 9158 | GGCGGCGGCTGCGTGGTGGT | 3294 |
| 9159 | GCGGCGGCTGCGTGGTGGTG | 3295 |
| 9160 | CGGCGGCTGCGTGGTGGTGG | 3296 |
| 9161 | GGCGGCTGCGTGGTGGTGGC | 3297 |
| 9162 | GCGGCTGCGTGGTGGTGGCG | 3298 |
| 9163 | CGGCTGCGTGGTGGTGGCGG | 3299 |
| 9164 | GGCTGCGTGGTGGTGGCGGG | 3300 |
| 9165 | GCTGCGTGGTGGTGGCGGGC | 3301 |
| 9166 | CTGCGTGGTGGTGGCGGGCG | 3302 |
| 9167 | TGCGTGGTGGTGGCGGGCG | 3303 |
| 9168 | GCGTGGTGGTGGCGGGCG | 3304 |
| 9169 | CGTGGTGGTGGCGGGCG | 3305 |
| 9170 | GTGGTGGTGGCGGGCG | 3306 |
| 9171 | TGGTGGTGGCGGGCG | 3307 |
| 9172 | GCGTGGGCGGCGGCGGCTGC | 3286 |
| 9173 | GGCGTGGGCGGCGGCGGCTG | 3285 |
| 9174 | CGGCGTGGGCGGCGGCGGCT | 3284 |
| 9175 | CCGGCGTGGGCGGCGGCGGC | 3283 |
| 9176 | GCCGGCGTGGGCGGCGGCGG | 3282 |
| 9177 | GGCCGGCGTGGGCGGCGGCG | 3281 |
| 9178 | AGGCCGGCGTGGGCGGCGGC | 3280 |

Hot Zones (Relative upstream location to gene start site)
1-630
3061-3321

### Examples

Genetic Code (5′ Upstream Region)

(SEQ ID NO: 11979)

CGCCCGCCACCACCACGCAGCCGCCGCCGCCCACGCCGGCCTCTGCTGCC
CCCTTCCCCAGCCCTTAGCACAGAGAGGGACACATGCCCCTCCCCCAGCT
GCGTTTTTTTATAGTAGATTTTTAACAAAAAACGGGGAGAAATAATGCAT
TTCTGTGGATACAGTGCCCACCGCCCTCCTCCACTTGGAAACGGTATCCT
CCCTGCCCATCCGTCTGTCTGTCGCCCTTCTCCCGGCCCTCACTAAGCCC
CGGCACTTCTAGTGGTCTCACCTGGAGGCAAGAGGGAGGGGACAGAGGCC
CTGCCACGTCCCGCTGCCTCCTGCTCTCTGGAGGTACTGAGACAGGGTGC
TGATGGGAAGGAGGGGAGCCTTTGGGGGGCCACCCGGGGCCTGGACCTAT
GCAGGGAGGCCACGTCCCACCCCACCTCTTGTTTCTGGGTCCCTGCTCCC
CTTTGGGGGTGTGTGTGTGTGTTTTAATTTTCTTTATGGAAAAATTGACA
AAAAAAAAATAGAGAGAGAGGTATTTAACTGCAATAAACTGGCCCCATGT
GGCCCCCGCCTTGTCTGCTTGTGTGTTTGTCCATCTCAGGAGTGGGGAGG
GGGCCTGGGGTCTGCAGAGCTCCACGAGGCATGGTTCTGCTGTTGTGCAC
ATGGCTGTGCATGGTCCCTGCCAGCTGCACCACCCATTACCCAGTGGTTG
GTTGGATGGATGGAGGAATTAAGGAATGAATGTCCCCTTTGAGGCCCTAG
ACGTGCATGAGGGTGTGGGGAGCTGGGGTCAAGGACATGTCCCATGTTGG
AGGAGAGGCAGGGGTCTCCGTGTCAACAGTTCCTGAAAACACAACCAGCC
CCTGGCCCTGCCCTGCTGGGCCAAAGCCCTCCCCTCTGCACCAGCCAATA
GTGGGGCCTGGCCTTGAGCCCCTCACCCCCAGGGAGGGCAGATGGCCAGG
GCGCCAAGCTTGGCCCGTCAGCCTGTCGCCTTGCACCAAGGCTCTGGCGC
CTGTGCTGTGACCCCTGCCCCTGCTGATGATGAAACCTGTCCTCAGCTGA
GATGCAGCGATGCCTGGTAGGGCTGGGGGCTGCTCCTGTGTCTCCCCAGG
TGAGCACACCCCTATTCACTGGGCCCTGCTTCAGCCTGCAGCACCCTTCA
ACTCCCAGGAGCTGGGCTTGCCACTCTGCTCACCTTGTGGAGCTCCATCT
GCCTTTCCTCCCCAATTCCCCCACTCCCTGCACTCGTCTCTTCCCACAAG
AGCCCTGTCTCCTTTTCCTAGCTATTCCCATCTGAGGCCATCTTTATTCA
TTTAGTTTTTAGAGACAGGGTTTCACTCTCACCCAGGCTGGGGTGCAGTG
GCACACAATCACGGCTCACTGCAGCCTTGACCAACTACAGGTGCGTAGCA
CCACAGCCAAGTTTTTGTATAGATGGGGTCTCGCTTTGTTACCCAGGCTG
TGACAAGAGGAGCCTCCCACGTGGTGTGGATGAGGAGGCAGATGGCAGGG
CCTGTGCATTTCTGTGCTTGAGTGGGCCTTGAAAGTGGTTCAGCAACCAG
GAAGAAGTGTTCATTCCTCGACAACAACATCCCCGGGCTCTGGTGACTTG
GCTGACACTGGATGGCCCTGGAATGAAAAAGGCAAAGAGGCAAAATGTGC
AAGGGCCCATCTGGAACCAAGGTTTGTTGATCCCCTGGGCCGTGTGCACC
CTGAGCTGGGCCTGGTAGTGGAAAGGAATGAAGGCACTGCAGTCAGGCAG
CCTGGGTTCATCCCCCAGCTAGTGGTGTCCTAAGGAACCGGCTCCCCAAA
AACATCCCTGGCTTGTAGTGCTTGCCAATTTCTGGGTGTCAAGACTCCCA
CTGCTGCTGATTTCAGGATACCAGCATGATGCCACTGAATGCAGAGTTTC
GAGATGTGCATGGTCTGCTATGTTGAGCCAGGTCTAGCATACCGCTGTGC
CCTGCTGTGTTTTAGGGGAGATGGGGAAACCTGGTGGGTAAGAGCAAAAG
CCCTGGAGTCAGGCTGTCCAGGCTAGAATCTCAGCTCTGCCTCTGGCTGA
GCAAGCTTGGGCCATGCCCTGATCTCTGCCTTCAGTGCCTTTTCTGTAAA
GTGAAGGAAATGAGTGTCCGACGGGGAGGAGGTTCCTAAAAGGGAGCAGG
GTCTGGGGAGCCCAGGCCTCTGGGGTTGGGTGACTGAGAAGGCAGCCCCT
GAATACAGAGCAGAGCTGAAGGTGGGGCAGTAAGTGCTGCTGGGAGAACA
GGCAGCACAGGCTGAGTTGGTGCAGAAGTGAGTCAACATATGTGCCATCG
TATAAAATGTACTCATCGGACTGTAGATGTTAGCTATTACTATTACTGCT
ATTTTATGTTTTATAGACAGGGTCTCACTCTGTCACCCAGGCTGGAGTGC
AGTCACACAATCATAGCTCACTGCAACCTCAGCCTCCTGGGCTTAAGCGA
TCTGCCTCAGCCTCCCAAGTAGCTGGGACTACAGATGTGTGCCACCACGC
CTGGCTAAATTTGTTTAAAATTTTTTTTGTAGAGATGGGGTCTCCCTATG
TTGCCCAGGCTAGTCTTGAACTTCTGGGCTCAAGCGACCCTCCTGCCTTG
GCCTCCCAAATTGCTGGGATTACAGGCATAAGCCACTGTGCTGGGCCATA
TTACTGCTGTCATTTATGGCCAAAAGTTTGCTCAAACATTTTCCAGTTAC
CAGAGCCACATCTCAAGGGTCTGACACTGGGAAAACACCACGTGCGGATC
GGGCACACGCTGATGCTTGCCCTGCTCAGGGCTATCTAGTGTTCCCTGCC
AGAACCTATGCACGTGTGGTGAGAGCTTAAAGCAATGGATGCTTCCCCCA
ACATGCCAGACACTCCTGAGGAGCCTGGCGGCTGCTGGCCATGCCCCGTG
TGCATGTAGGCGATGGGGAAGTGAGTGGAGGAGAGCGGAACCTTGATTCT
GCTCATCAAACTGCTTAACCGCTGAAGCAAAAGGGGGAACTTTTTTCCCG
ATCAGCAGAATGACATCGTGATGGGGAAAGGGCTCCCCAGATGGCTGGTG
AGCAGTGTGTGTCTGTGACCCCGTCTGCCCCACCCCCTGAACACACCTCT
GCCGGCTGAGGGTGACACAACCCTGTTCCCTGTCGCTCTGTTCCCGCTTA
TCTCTCCCGCCTTTTCGGCGCCACCACCTTCTTGGAAATGAGACAGAGCA
AAGGGGAGGGGGCTCAGACCACCGCCTCCCCTGGCAGGCCCCATAAAAGC
GACTGTCACTCGGTCCCAGACACCAGAGCAAGCTCAAGACCCAGCAGTGG
GACAGCCAGACAGACGGCACGATG

HBV

Hepatitis B is an infectious inflammatory disease of the liver caused by the hepatitis B virus (HBV). About ⅓ of the world population is believed to be infected, including 350 million who are chronic carriers. Acutely symptoms include liver inflammation, vomiting and jaundice, while chronic hepatitis B is implicated in cirrhosis and liver cancer. HBV is a DNA virus that has a circular genome of partially double-stranded DNA (Zuckerman A. J. 1996 in Baron S, et al. Baron's Medical Microbiology (4th Ed)) with a full length strand with 3020-3320 nucleotides and a short-length strand of 1700-2800 nucleotides for the short length-strand (Kay A and Zoulim F. 2007 Virus research 127 (2): 164-176). HBV uses reverse transcription to replicate: virus gains entry into the cell by endocytosis, multiplies via RNA made by a host enzyme, then reversed transcribed into viral genomic DNA. The partially double stranded viral DNA is rendered fully double stranded when transformed into covalently closed circular DNA (cccDNA). cccDNA serves as a template for transcription of four viral mRNAs encoding viral proteins called C, X, P and S critical of virus infection and replication. HBV core protein is coded for by gene C (HBcAg); its DNA polymerase is encoded by gene P; the surface antigen (HBsAg) is encoded by the S gene. HBx protein is encoded by the X gene and is believed to drive cccDNA transcription and stimulates genes to promote cell growth associated with liver cancer and the persistence of HBV.

Hepatitis B Virus (1-3215 [NCBI Reference Sequence: NC_{—}003977]; strand: negative)

Targeted Sequences

| | | | Relative |
|---|---|---|---|
| | | | upstream |
| | | | location |
| | | | to |
| Sequence | Design | | start |
| ID No: | ID | Sequence (5′-3′) | site |
| 9179 | | CCGATTGGTGGAGGCAGGAGGAGG | 72 |
| 9180 | | CGAGATTGAGATCTTCTGCGACGCGG | 780 |
| 9235 | | CGCGGCGATTGAGACCTTCGTC | 801 |
| 9290 | | CGTCTGCGAGGCGAGGGAGTTCTTCT | 819 |
| 9345 | | CGATACAGAGCAGAGGCGGTGT | 1200 |
| 9346 | | CGCGTAAAGAGAGGTGCGCCCCGTGG | 1674 |
| 9360 | | ACGGGTCGTCCGCGGGATTCAGCGCCG | 1754 |
| 9409 | | CGTCCCGCGCAGGATCCAGTTGG | 1800 |
| 9432 | | CGGCTGCGAGCAAAACAAGCTGCTAG | 1909 |
| 9468 | | CGCATGCGCCGATGGCCTATGGCCAA | 1978 |
| 9496 | | CGCCGCAGACACATCCAGCGATA | 2826 |
| 9525 | | GCTCCAGACCGGCTGCGA | 1900 |
| 9561 | | CGTCCATCGCAGGATCCAGTTGG | 1800 |
| 9562 | | CGCCGCAGACACATCCAGCGATA | 2826 |
| 9591 | | CAAATGGCACTAGTAAACTGAG | 2524 |
| 9592 | | GAGATTGAGATCTGCGGCGACGCGG | 780 |
| 9593 | | CGACGCGGCGATTGAGATCTTCGTCTG | 801 |
| 9594 | | AGGGGTCGTCCGCGGGATTCAGCGCCG | 1754 |

Target Shift Sequences

| | | Relative |
|---|---|---|
| | | upstream |
| Sequence | | location |
| ID No: | Sequence (5′-3′) | to start site |
| 9179 | CCGATTGGTGGAGGCAGGAGGAGG | 72 |
| 9180 | CGAGATTGAGATCTTCTGCGACGCGG | 780 |
| 9181 | GAGATTGAGATCTTCTGCGA | 781 |
| 9182 | AGATTGAGATCTTCTGCGAC | 782 |
| 9183 | GATTGAGATCTTCTGCGACG | 783 |
| 9184 | ATTGAGATCTTCTGCGACGC | 784 |
| 9185 | TTGAGATCTTCTGCGACGCG | 785 |
| 9186 | TGAGATCTTCTGCGACGCGG | 786 |
| 9187 | GAGATCTTCTGCGACGCGGC | 787 |
| 9188 | AGATCTTCTGCGACGCGGCG | 788 |
| 9189 | GATCTTCTGCGACGCGGCGA | 789 |
| 9190 | ATCTTCTGCGACGCGGCGAT | 790 |
| 9191 | TCTTCTGCGACGCGGCGATT | 791 |
| 9192 | CTTCTGCGACGCGGCGATTG | 792 |
| 9193 | TTCTGCGACGCGGCGATTGA | 793 |
| 9194 | TCTGCGACGCGGCGATTGAG | 794 |
| 9195 | CTGCGACGCGGCGATTGAGA | 795 |
| 9196 | TGCGACGCGGCGATTGAGAC | 796 |
| 9197 | GCGACGCGGCGATTGAGACC | 797 |
| 9198 | CGACGCGGCGATTGAGACCT | 798 |
| 9199 | GACGCGGCGATTGAGACCTT | 799 |
| 9200 | ACGCGGCGATTGAGACCTTC | 800 |
| 9201 | CGCGGCGATTGAGACCTTCG | 801 |
| 9202 | GCGGCGATTGAGACCTTCGT | 802 |
| 9203 | CGGCGATTGAGACCTTCGTC | 803 |
| 9204 | GGCGATTGAGACCTTCGTCT | 804 |
| 9205 | GCGATTGAGACCTTCGTCTG | 805 |
| 9206 | CGATTGAGACCTTCGTCTGC | 806 |
| 9207 | GATTGAGACCTTCGTCTGCG | 807 |
| 9208 | ATTGAGACCTTCGTCTGCGA | 808 |
| 9209 | TTGAGACCTTCGTCTGCGAG | 809 |
| 9210 | TGAGACCTTCGTCTGCGAGG | 810 |
| 9211 | GAGACCTTCGTCTGCGAGGC | 811 |
| 9212 | AGACCTTCGTCTGCGAGGCG | 812 |
| 9213 | GACCTTCGTCTGCGAGGCGA | 813 |
| 9214 | ACCTTCGTCTGCGAGGCGAG | 814 |
| 9215 | CCTTCGTCTGCGAGGCGAGG | 815 |
| 9216 | CTTCGTCTGCGAGGCGAGGG | 816 |
| 9217 | TTCGTCTGCGAGGCGAGGGA | 817 |
| 9218 | TCGTCTGCGAGGCGAGGGAG | 818 |
| 9219 | CGTCTGCGAGGCGAGGGAGT | 819 |
| 9220 | GTCTGCGAGGCGAGGGAGTT | 820 |
| 9221 | TCTGCGAGGCGAGGGAGTTC | 821 |
| 9222 | CTGCGAGGCGAGGGAGTTCT | 822 |
| 9223 | TGCGAGGCGAGGGAGTTCTT | 823 |
| 9224 | GCGAGGCGAGGGAGTTCTTC | 824 |
| 9225 | CGAGGCGAGGGAGTTCTTCT | 825 |
| 9226 | GAGGCGAGGGAGTTCTTCTT | 826 |
| 9227 | AGGCGAGGGAGTTCTTCTTC | 827 |
| 9228 | GGCGAGGGAGTTCTTCTTCT | 828 |
| 9229 | GCGAGGGAGTTCTTCTTCTA | 829 |
| 9230 | CGAGGGAGTTCTTCTTCTAG | 830 |
| 9231 | CCGAGATTGAGATCTTCTGC | 779 |
| 9232 | CCCGAGATTGAGATCTTCTG | 778 |
| 9233 | TCCCGAGATTGAGATCTTCT | 777 |
| 9234 | TTCCCGAGATTGAGATCTTC | 776 |
| 9235 | CGCGGCGATTGAGACCTTCGTC | 801 |
| 9236 | GCGGCGATTGAGACCTTCGT | 802 |
| 9237 | CGGCGATTGAGACCTTCGTC | 803 |
| 9238 | GGCGATTGAGACCTTCGTCT | 804 |
| 9239 | GCGATTGAGACCTTCGTCTG | 805 |
| 9240 | CGATTGAGACCTTCGTCTGC | 806 |
| 9241 | GATTGAGACCTTCGTCTGCG | 807 |
| 9242 | ATTGAGACCTTCGTCTGCGA | 808 |
| 9243 | TTGAGACCTTCGTCTGCGAG | 809 |
| 9244 | TGAGACCTTCGTCTGCGAGG | 810 |
| 9245 | GAGACCTTCGTCTGCGAGGC | 811 |
| 9246 | AGACCTTCGTCTGCGAGGCG | 812 |
| 9247 | GACCTTCGTCTGCGAGGCGA | 813 |
| 9248 | ACCTTCGTCTGCGAGGCGAG | 814 |
| 9249 | CCTTCGTCTGCGAGGCGAGG | 815 |
| 9250 | CTTCGTCTGCGAGGCGAGGG | 816 |
| 9251 | TTCGTCTGCGAGGCGAGGGA | 817 |
| 9252 | TCGTCTGCGAGGCGAGGGAG | 818 |
| 9253 | CGTCTGCGAGGCGAGGGAGT | 819 |
| 9254 | GTCTGCGAGGCGAGGGAGTT | 820 |
| 9255 | TCTGCGAGGCGAGGGAGTTC | 821 |
| 9256 | CTGCGAGGCGAGGGAGTTCT | 822 |
| 9257 | TGCGAGGCGAGGGAGTTCTT | 823 |
| 9258 | GCGAGGCGAGGGAGTTCTTC | 824 |
| 9259 | CGAGGCGAGGGAGTTCTTCT | 825 |
| 9260 | GAGGCGAGGGAGTTCTTCTT | 826 |
| 9261 | AGGCGAGGGAGTTCTTCTTC | 827 |
| 9262 | GGCGAGGGAGTTCTTCTTCT | 828 |
| 9263 | GCGAGGGAGTTCTTCTTCTA | 829 |
| 9264 | CGAGGGAGTTCTTCTTCTAG | 830 |
| 9265 | ACGCGGCGATTGAGACCTTC | 800 |
| 9266 | GACGCGGCGATTGAGACCTT | 799 |
| 9267 | CGACGCGGCGATTGAGACCT | 798 |
| 9268 | GCGACGCGGCGATTGAGACC | 797 |
| 9269 | TGCGACGCGGCGATTGAGAC | 796 |
| 9270 | CTGCGACGCGGCGATTGAGA | 795 |
| 9271 | TCTGCGACGCGGCGATTGAG | 794 |
| 9272 | TTCTGCGACGCGGCGATTGA | 793 |
| 9273 | CTTCTGCGACGCGGCGATTG | 792 |
| 9274 | TCTTCTGCGACGCGGCGATT | 791 |
| 9275 | ATCTTCTGCGACGCGGCGAT | 790 |
| 9276 | GATCTTCTGCGACGCGGCGA | 789 |
| 9277 | AGATCTTCTGCGACGCGGCG | 788 |
| 9278 | GAGATCTTCTGCGACGCGGC | 787 |
| 9279 | TGAGATCTTCTGCGACGCGG | 786 |
| 9280 | TTGAGATCTTCTGCGACGCG | 785 |
| 9281 | ATTGAGATCTTCTGCGACGC | 784 |
| 9282 | GATTGAGATCTTCTGCGACG | 783 |
| 9283 | AGATTGAGATCTTCTGCGAC | 782 |
| 9284 | GAGATTGAGATCTTCTGCGA | 781 |
| 9285 | CGAGATTGAGATCTTCTGCG | 780 |
| 9286 | CCGAGATTGAGATCTTCTGC | 779 |
| 9287 | CCCGAGATTGAGATCTTCTG | 778 |
| 9288 | TCCCGAGATTGAGATCTTCT | 777 |
| 9289 | TTCCCGAGATTGAGATCTTC | 776 |
| 9290 | CGTCTGCGAGGCGAGGGAGTTCTTCT | 819 |
| 9291 | GTCTGCGAGGCGAGGGAGTT | 820 |
| 9292 | TCTGCGAGGCGAGGGAGTTC | 821 |
| 9293 | CTGCGAGGCGAGGGAGTTCT | 822 |
| 9294 | TGCGAGGCGAGGGAGTTCTT | 823 |
| 9295 | GCGAGGCGAGGGAGTTCTTC | 824 |
| 9296 | CGAGGCGAGGGAGTTCTTCT | 825 |
| 9297 | GAGGCGAGGGAGTTCTTCTT | 826 |
| 9298 | AGGCGAGGGAGTTCTTCTTC | 827 |
| 9299 | GGCGAGGGAGTTCTTCTTCT | 828 |
| 9300 | GCGAGGGAGTTCTTCTTCTA | 829 |
| 9301 | CGAGGGAGTTCTTCTTCTAG | 830 |
| 9302 | TCGTCTGCGAGGCGAGGGAG | 818 |
| 9303 | TTCGTCTGCGAGGCGAGGGA | 817 |
| 9304 | CTTCGTCTGCGAGGCGAGGG | 816 |
| 9305 | CCTTCGTCTGCGAGGCGAGG | 815 |
| 9306 | ACCTTCGTCTGCGAGGCGAG | 814 |
| 9307 | GACCTTCGTCTGCGAGGCGA | 813 |
| 9308 | AGACCTTCGTCTGCGAGGCG | 812 |
| 9309 | GAGACCTTCGTCTGCGAGGC | 811 |
| 9310 | TGAGACCTTCGTCTGCGAGG | 810 |
| 9311 | TTGAGACCTTCGTCTGCGAG | 809 |
| 9312 | ATTGAGACCTTCGTCTGCGA | 808 |
| 9313 | GATTGAGACCTTCGTCTGCG | 807 |
| 9314 | CGATTGAGACCTTCGTCTGC | 806 |
| 9315 | GCGATTGAGACCTTCGTCTG | 805 |
| 9316 | GGCGATTGAGACCTTCGTCT | 804 |
| 9317 | CGGCGATTGAGACCTTCGTC | 803 |
| 9318 | GCGGCGATTGAGACCTTCGT | 802 |
| 9319 | CGCGGCGATTGAGACCTTCG | 801 |
| 9320 | ACGCGGCGATTGAGACCTTC | 800 |
| 9321 | GACGCGGCGATTGAGACCTT | 799 |
| 9322 | CGACGCGGCGATTGAGACCT | 798 |
| 9323 | GCGACGCGGCGATTGAGACC | 797 |
| 9324 | TGCGACGCGGCGATTGAGAC | 796 |
| 9325 | CTGCGACGCGGCGATTGAGA | 795 |
| 9326 | TCTGCGACGCGGCGATTGAG | 794 |
| 9327 | TTCTGCGACGCGGCGATTGA | 793 |
| 9328 | CTTCTGCGACGCGGCGATTG | 792 |
| 9329 | TCTTCTGCGACGCGGCGATT | 791 |
| 9330 | ATCTTCTGCGACGCGGCGAT | 790 |
| 9331 | GATCTTCTGCGACGCGGCGA | 789 |
| 9332 | AGATCTTCTGCGACGCGGCG | 788 |
| 9333 | GAGATCTTCTGCGACGCGGC | 787 |
| 9334 | TGAGATCTTCTGCGACGCGG | 786 |
| 9335 | TTGAGATCTTCTGCGACGCG | 785 |
| 9336 | ATTGAGATCTTCTGCGACGC | 784 |
| 9337 | GATTGAGATCTTCTGCGACG | 783 |
| 9338 | AGATTGAGATCTTCTGCGAC | 782 |
| 9339 | GAGATTGAGATCTTCTGCGA | 781 |
| 9340 | CGAGATTGAGATCTTCTGCG | 780 |
| 9341 | CCGAGATTGAGATCTTCTGC | 779 |
| 9342 | CCCGAGATTGAGATCTTCTG | 778 |
| 9343 | TCCCGAGATTGAGATCTTCT | 777 |
| 9344 | TTCCCGAGATTGAGATCTTC | 776 |
| 9345 | CGATACAGAGCAGAGGCGGTGT | 1200 |
| 9346 | CGCGTAAAGAGAGGTGCGCCCCGTGG | 1674 |
| 9347 | GCGTAAAGAGAGGTGCGCCC | 1675 |
| 9348 | CGTAAAGAGAGGTGCGCCCC | 1676 |
| 9349 | GTAAAGAGAGGTGCGCCCCG | 1677 |
| 9350 | TAAAGAGAGGTGCGCCCCGT | 1678 |
| 9351 | AAAGAGAGGTGCGCCCCGTG | 1679 |
| 9352 | AAGAGAGGTGCGCCCCGTGG | 1680 |
| 9353 | AGAGAGGTGCGCCCCGTGGT | 1681 |
| 9354 | GAGAGGTGCGCCCCGTGGTC | 1682 |
| 9355 | AGAGGTGCGCCCCGTGGTCG | 1683 |
| 9356 | GAGGTGCGCCCCGTGGTCGG | 1684 |
| 9357 | AGGTGCGCCCCGTGGTCGGC | 1685 |
| 9358 | GGTGCGCCCCGTGGTCGGCC | 1686 |
| 9359 | CCGCGTAAAGAGAGGTGCGC | 1673 |
| 9360 | ACGGGTCGTCCGCGGGATTCAGCGCCG | 1754 |
| 9361 | CGGGTCGTCCGCGGGATTCA | 1755 |
| 9362 | GGGTCGTCCGCGGGATTCAG | 1756 |
| 9363 | GGTCGTCCGCGGGATTCAGC | 1757 |
| 9364 | GTCGTCCGCGGGATTCAGCG | 1758 |
| 9365 | TCGTCCGCGGGATTCAGCGC | 1759 |
| 9366 | CGTCCGCGGGATTCAGCGCC | 1760 |
| 9367 | GTCCGCGGGATTCAGCGCCG | 1761 |
| 9368 | TCCGCGGGATTCAGCGCCGA | 1762 |
| 9369 | CCGCGGGATTCAGCGCCGAC | 1763 |
| 9370 | CGCGGGATTCAGCGCCGACG | 1764 |
| 9371 | GCGGGATTCAGCGCCGACGG | 1765 |
| 9372 | CGGGATTCAGCGCCGACGGG | 1766 |
| 9373 | GGGATTCAGCGCCGACGGGA | 1767 |
| 9374 | GGATTCAGCGCCGACGGGAC | 1768 |
| 9375 | GATTCAGCGCCGACGGGACG | 1769 |
| 9376 | ATTCAGCGCCGACGGGACGT | 1770 |
| 9377 | TTCAGCGCCGACGGGACGTA | 1771 |
| 9378 | TCAGCGCCGACGGGACGTAG | 1772 |
| 9379 | CAGCGCCGACGGGACGTAGA | 1773 |
| 9380 | AGCGCCGACGGGACGTAGAC | 1774 |
| 9381 | GCGCCGACGGGACGTAGACA | 1775 |
| 9382 | CGCCGACGGGACGTAGACAA | 1776 |
| 9383 | GACGGGTCGTCCGCGGGATT | 1753 |
| 9384 | AGACGGGTCGTCCGCGGGAT | 1752 |
| 9385 | GAGACGGGTCGTCCGCGGGA | 1751 |
| 9386 | CGAGACGGGTCGTCCGCGGG | 1750 |
| 9387 | CCGAGACGGGTCGTCCGCGG | 1749 |
| 9388 | CCCGAGACGGGTCGTCCGCG | 1748 |
| 9389 | CCCCGAGACGGGTCGTCCGC | 1747 |
| 9390 | GCCCCGAGACGGGTCGTCCG | 1746 |
| 9391 | GGCCCCGAGACGGGTCGTCC | 1745 |
| 9392 | CGGCCCCGAGACGGGTCGTC | 1744 |
| 9393 | ACGGCCCCGAGACGGGTCGT | 1743 |
| 9394 | AACGGCCCCGAGACGGGTCG | 1742 |
| 9395 | AAACGGCCCCGAGACGGGTC | 1741 |
| 9396 | CAAACGGCCCCGAGACGGGT | 1740 |
| 9397 | CCAAACGGCCCCGAGACGGG | 1739 |
| 9398 | CCCAAACGGCCCCGAGACGG | 1738 |
| 9399 | GCCCAAACGGCCCCGAGACG | 1737 |
| 9400 | GGCCCAAACGGCCCCGAGAC | 1736 |
| 9401 | AGGCCCAAACGGCCCCGAGA | 1735 |
| 9402 | GAGGCCCAAACGGCCCCGAG | 1734 |
| 9403 | AGAGGCCCAAACGGCCCCGA | 1733 |
| 9404 | TAGAGGCCCAAACGGCCCCG | 1732 |
| 9405 | GTAGAGGCCCAAACGGCCCC | 1731 |
| 9406 | GGTAGAGGCCCAAACGGCCC | 1730 |
| 9407 | CGGTAGAGGCCCAAACGGCC | 1729 |
| 9408 | ACGGTAGAGGCCCAAACGGC | 1728 |
| 9409 | CGTCCCGCGCAGGATCCAGTTGG | 1800 |
| 9410 | GTCCCGCGCAGGATCCAGTT | 1801 |
| 9411 | TCCCGCGCAGGATCCAGTTG | 1802 |
| 9412 | CCCGCGCAGGATCCAGTTGG | 1803 |
| 9413 | CCGCGCAGGATCCAGTTGGC | 1804 |
| 9414 | CGCGCAGGATCCAGTTGGCA | 1805 |
| 9415 | GCGCAGGATCCAGTTGGCAG | 1806 |
| 9416 | CGCAGGATCCAGTTGGCAGC | 1807 |
| 9417 | ACGTCCCGCGCAGGATCCAG | 1799 |
| 9418 | GACGTCCCGCGCAGGATCCA | 1798 |
| 9419 | GGACGTCCCGCGCAGGATCC | 1797 |
| 9420 | AGGACGTCCCGCGCAGGATC | 1796 |
| 9421 | AAGGACGTCCCGCGCAGGAT | 1795 |
| 9422 | AAAGGACGTCCCGCGCAGGA | 1794 |
| 9423 | CAAAGGACGTCCCGCGCAGG | 1793 |
| 9424 | ACAAAGGACGTCCCGCGCAG | 1792 |
| 9425 | GACAAAGGACGTCCCGCGCA | 1791 |
| 9426 | AGACAAAGGACGTCCCGCGC | 1790 |
| 9427 | TAGACAAAGGACGTCCCGCG | 1789 |
| 9428 | GTAGACAAAGGACGTCCCGC | 1788 |
| 9429 | CGTAGACAAAGGACGTCCCG | 1787 |
| 9430 | ACGTAGACAAAGGACGTCCC | 1786 |
| 9431 | GACGTAGACAAAGGACGTCC | 1785 |
| 9432 | CGGCTGCGAGCAAAACAAGCTGCTAG | 1909 |
| 9433 | GGCTGCGAGCAAAACAAGCT | 1910 |
| 9434 | GCTGCGAGCAAAACAAGCTG | 1911 |
| 9435 | CTGCGAGCAAAACAAGCTGC | 1912 |
| 9436 | TGCGAGCAAAACAAGCTGCT | 1913 |
| 9437 | GCGAGCAAAACAAGCTGCTA | 1914 |
| 9438 | CGAGCAAAACAAGCTGCTAG | 1915 |
| 9439 | CCGGCTGCGAGCAAAACAAG | 1908 |
| 9440 | ACCGGCTGCGAGCAAAACAA | 1907 |
| 9441 | GACCGGCTGCGAGCAAAACA | 1906 |
| 9442 | AGACCGGCTGCGAGCAAAAC | 1905 |
| 9443 | CAGACCGGCTGCGAGCAAAA | 1904 |
| 9444 | CCAGACCGGCTGCGAGCAAA | 1903 |
| 9445 | TCCAGACCGGCTGCGAGCAA | 1902 |
| 9446 | CTCCAGACCGGCTGCGAGCA | 1901 |
| 9447 | GCTCCAGACCGGCTGCGAGC | 1900 |
| 9448 | CGCTCCAGACCGGCTGCGAG | 1899 |
| 9449 | TCGCTCCAGACCGGCTGCGA | 1898 |
| 9450 | TTCGCTCCAGACCGGCTGCG | 1897 |
| 9451 | TTTCGCTCCAGACCGGCTGC | 1896 |
| 9452 | GTTTCGCTCCAGACCGGCTG | 1895 |
| 9453 | AGTTTCGCTCCAGACCGGCT | 1894 |
| 9454 | AAGTTTCGCTCCAGACCGGC | 1893 |
| 9455 | TAAGTTTCGCTCCAGACCGG | 1892 |
| 9456 | ATAAGTTTCGCTCCAGACCG | 1891 |
| 9457 | GATAAGTTTCGCTCCAGACC | 1890 |
| 9458 | CGATAAGTTTCGCTCCAGAC | 1889 |
| 9459 | CCGATAAGTTTCGCTCCAGA | 1888 |
| 9460 | TCCGATAAGTTTCGCTCCAG | 1887 |
| 9461 | TTCCGATAAGTTTCGCTCCA | 1886 |
| 9462 | GTTCCGATAAGTTTCGCTCC | 1885 |
| 9463 | GGTTCCGATAAGTTTCGCTC | 1884 |
| 9464 | CGGTTCCGATAAGTTTCGCT | 1883 |
| 9465 | TCGGTTCCGATAAGTTTCGC | 1882 |
| 9466 | GTCGGTTCCGATAAGTTTCG | 1881 |
| 9467 | TGTCGGTTCCGATAAGTTTC | 1880 |
| 9468 | CGCATGCGCCGATGGCCTATGGCCAA | 1978 |
| 9469 | GCATGCGCCGATGGCCTATG | 1979 |
| 9470 | CATGCGCCGATGGCCTATGG | 1980 |
| 9471 | ATGCGCCGATGGCCTATGGC | 1981 |
| 9472 | TGCGCCGATGGCCTATGGCC | 1982 |
| 9473 | GCGCCGATGGCCTATGGCCA | 1983 |
| 9474 | CGCCGATGGCCTATGGCCAA | 1984 |
| 9475 | GCCGATGGCCTATGGCCAAG | 1985 |
| 9476 | CCGATGGCCTATGGCCAAGC | 1986 |
| 9477 | CGATGGCCTATGGCCAAGCC | 1987 |
| 9478 | ACGCATGCGCCGATGGCCTA | 1977 |
| 9479 | CACGCATGCGCCGATGGCCT | 1976 |
| 9480 | CCACGCATGCGCCGATGGCC | 1975 |
| 9481 | TCCACGCATGCGCCGATGGC | 1974 |
| 9482 | TTCCACGCATGCGCCGATGG | 1973 |
| 9483 | GTTCCACGCATGCGCCGATG | 1972 |
| 9484 | GGTTCCACGCATGCGCCGAT | 1971 |
| 9485 | AGGTTCCACGCATGCGCCGA | 1970 |
| 9486 | AAGGTTCCACGCATGCGCCG | 1969 |
| 9487 | AAAGGTTCCACGCATGCGCC | 1968 |
| 9488 | CAAAGGTTCCACGCATGCGC | 1967 |
| 9489 | ACAAAGGTTCCACGCATGCG | 1966 |
| 9490 | CACAAAGGTTCCACGCATGC | 1965 |
| 9491 | CCACAAAGGTTCCACGCATG | 1964 |
| 9492 | GCCACAAAGGTTCCACGCAT | 1963 |
| 9493 | AGCCACAAAGGTTCCACGCA | 1962 |
| 9494 | GAGCCACAAAGGTTCCACGC | 1961 |
| 9495 | GGAGCCACAAAGGTTCCACG | 1960 |
| 9496 | CGCCGCAGACACATCCAGCGATA | 2826 |
| 9497 | GCCGCAGACACATCCAGCGA | 2827 |
| 9498 | CCGCAGACACATCCAGCGAT | 2828 |
| 9499 | CGCAGACACATCCAGCGATA | 2829 |
| 9500 | GCAGACACATCCAGCGATAG | 2830 |
| 9501 | CAGACACATCCAGCGATAGC | 2831 |
| 9502 | AGACACATCCAGCGATAGCC | 2832 |
| 9503 | GACACATCCAGCGATAGCCA | 2833 |
| 9504 | ACACATCCAGCGATAGCCAG | 2834 |
| 9505 | CACATCCAGCGATAGCCAGG | 2835 |
| 9506 | ACATCCAGCGATAGCCAGGA | 2836 |
| 9507 | CATCCAGCGATAGCCAGGAC | 2837 |
| 9508 | ATCCAGCGATAGCCAGGACA | 2838 |
| 9509 | TCCAGCGATAGCCAGGACAA | 2839 |
| 9510 | CCAGCGATAGCCAGGACAAG | 2840 |
| 9511 | CAGCGATAGCCAGGACAAGT | 2841 |
| 9512 | AGCGATAGCCAGGACAAGTT | 2842 |
| 9513 | GCGATAGCCAGGACAAGTTG | 2843 |
| 9514 | CGATAGCCAGGACAAGTTGG | 2844 |
| 9515 | ACGCCGCAGACACATCCAGC | 2825 |
| 9516 | AACGCCGCAGACACATCCAG | 2824 |
| 9517 | AAACGCCGCAGACACATCCA | 2823 |
| 9518 | AAAACGCCGCAGACACATCC | 2822 |
| 9519 | TAAAACGCCGCAGACACATC | 2821 |
| 9520 | ATAAAACGCCGCAGACACAT | 2820 |
| 9521 | GATAAAACGCCGCAGACACA | 2819 |
| 9522 | TGATAAAACGCCGCAGACAC | 2818 |
| 9523 | ATGATAAAACGCCGCAGACA | 2817 |
| 9524 | TATGATAAAACGCCGCAGAC | 2816 |
| 9525 | GCTCCAGACCGGCTGCGA | 1900 |
| 9526 | CTCCAGACCGGCTGCGAGCA | 1901 |
| 9527 | TCCAGACCGGCTGCGAGCAA | 1902 |
| 9528 | CCAGACCGGCTGCGAGCAAA | 1903 |
| 9529 | CAGACCGGCTGCGAGCAAAA | 1904 |
| 9530 | AGACCGGCTGCGAGCAAAAC | 1905 |
| 9531 | GACCGGCTGCGAGCAAAACA | 1906 |
| 9532 | ACCGGCTGCGAGCAAAACAA | 1907 |
| 9533 | CCGGCTGCGAGCAAAACAAG | 1908 |
| 9534 | CGGCTGCGAGCAAAACAAGC | 1909 |
| 9535 | GGCTGCGAGCAAAACAAGCT | 1910 |
| 9536 | GCTGCGAGCAAAACAAGCTG | 1911 |
| 9537 | CTGCGAGCAAAACAAGCTGC | 1912 |
| 9538 | TGCGAGCAAAACAAGCTGCT | 1913 |
| 9539 | GCGAGCAAAACAAGCTGCTA | 1914 |
| 9540 | CGAGCAAAACAAGCTGCTAG | 1915 |
| 9541 | CGCTCCAGACCGGCTGCGAG | 1899 |
| 9542 | TCGCTCCAGACCGGCTGCGA | 1898 |
| 9543 | TTCGCTCCAGACCGGCTGCG | 1897 |
| 9544 | TTTCGCTCCAGACCGGCTGC | 1896 |
| 9545 | GTTTCGCTCCAGACCGGCTG | 1895 |
| 9546 | AGTTTCGCTCCAGACCGGCT | 1894 |
| 9547 | AAGTTTCGCTCCAGACCGGC | 1893 |
| 9548 | TAAGTTTCGCTCCAGACCGG | 1892 |
| 9549 | ATAAGTTTCGCTCCAGACCG | 1891 |
| 9550 | GATAAGTTTCGCTCCAGACC | 1890 |
| 9551 | CGATAAGTTTCGCTCCAGAC | 1889 |
| 9552 | CCGATAAGTTTCGCTCCAGA | 1888 |
| 9553 | TCCGATAAGTTTCGCTCCAG | 1887 |
| 9554 | TTCCGATAAGTTTCGCTCCA | 1886 |
| 9555 | GTTCCGATAAGTTTCGCTCC | 1885 |
| 9556 | GGTTCCGATAAGTTTCGCTC | 1884 |
| 9557 | CGGTTCCGATAAGTTTCGCT | 1883 |
| 9558 | TCGGTTCCGATAAGTTTCGC | 1882 |
| 9559 | GTCGGTTCCGATAAGTTTCG | 1881 |
| 9560 | TGTCGGTTCCGATAAGTTTC | 1880 |
| 9561 | CGTCCATCGCAGGATCCAGTTGG | 1800 |
| 9562 | CGCCGCAGACACATCCAGCGATA | 2826 |
| 9563 | GCCGCAGACACATCCAGCGA | 2827 |
| 9564 | CCGCAGACACATCCAGCGAT | 2828 |
| 9565 | CGCAGACACATCCAGCGATA | 2829 |
| 9566 | GCAGACACATCCAGCGATAG | 2830 |
| 9567 | CAGACACATCCAGCGATAGC | 2831 |
| 9568 | AGACACATCCAGCGATAGCC | 2832 |
| 9569 | GACACATCCAGCGATAGCCA | 2833 |
| 9570 | ACACATCCAGCGATAGCCAG | 2834 |
| 9571 | CACATCCAGCGATAGCCAGG | 2835 |
| 9572 | ACATCCAGCGATAGCCAGGA | 2836 |
| 9573 | CATCCAGCGATAGCCAGGAC | 2837 |
| 9574 | ATCCAGCGATAGCCAGGACA | 2838 |
| 9575 | TCCAGCGATAGCCAGGACAA | 2839 |
| 9576 | CCAGCGATAGCCAGGACAAG | 2840 |
| 9577 | CAGCGATAGCCAGGACAAGT | 2841 |
| 9578 | AGCGATAGCCAGGACAAGTT | 2842 |
| 9579 | GCGATAGCCAGGACAAGTTG | 2843 |
| 9580 | CGATAGCCAGGACAAGTTGG | 2844 |
| 9581 | ACGCCGCAGACACATCCAGC | 2825 |
| 9582 | AACGCCGCAGACACATCCAG | 2824 |
| 9583 | AAACGCCGCAGACACATCCA | 2823 |
| 9584 | AAAACGCCGCAGACACATCC | 2822 |
| 9585 | TAAAACGCCGCAGACACATC | 2821 |
| 9586 | ATAAAACGCCGCAGACACAT | 2820 |
| 9587 | GATAAAACGCCGCAGACACA | 2819 |
| 9588 | TGATAAAACGCCGCAGACAC | 2818 |
| 9589 | ATGATAAAACGCCGCAGACA | 2817 |
| 9590 | TATGATAAAACGCCGCAGAC | 2816 |
| 9591 | CAAATGGCACTAGTAAACTGAG | 2524 |
| 9592 | GAGATTGAGATCTGCGGCGACGCGG | 780 |
| 9593 | CGACGCGGCGATTGAGATCTTCGTCTG | 801 |
| 9594 | AGGGGTCGTCCGCGGGATTCAGCGCCG | 1754 |

Hot Zones (Relative upstream location to gene start site)
245-425
785-965
1145-1235
1505-2135
2585-3125

### Examples

Genetic Code (5′ Upstream Region)

(SEQ ID NO: 11980)

CTCCACAACATTCCACCAAGCTCTGCTAGATCCCAGAGTGAGGGGCCTAT
ATTTTCCTGCTGGTGGCTCCAGTTCCGGAACAGTAAACCCTGTTCCGACT
ACTGCCTCACCCATATCGTCAATCTTCTCGAGGACTGGGGACCCTGCACC
GAACATGGAGAGCACAACATCAGGATTCCTAGGACCCCTGCTCGTGTTAC
AGGCGGGGTTTTTCTTGTTGACAAGAATCCTCACAATACCACAGAGTCTA
GACTCGTGGTGGACTTCTCTCAATTTTCTAGGGGGAGCACCCACGTGTCC
TGGCCAAAATTCGCAGTCCCCAACCTCCAATCACTCACCAACCTCTTGTC
CTCCAACTTGTCCTGGCTATCGCTGGATGTGTCTGCGGCGTTTTATCATA
TTCCTCTTCATCCTGCTGCTATGCCTCATCTTCTTGTTGGTTCTTCTGGA
CTACCAAGGTATGTTGCCCGTTTGTCCTCTACTTCCAGGAACATCAACTA
CCAGCACGGGACCATGCAGAACCTGCACGATTCCTGCTCAAGGAACCTCT
ATGTTTCCCTCTTGTTGCTGTACAAAACCTTCGGACGGAAACTGCACTTG
TATTCCCATCCCATCATCCTGGGCTTTCGCAAGATTCCTATGGGAGTGGG
CCTCAGTCCGTTTCTCCTGGCTCAGTTTACTAGTGCCATTTGTTCAGTGG
TTCGTAGGGCTTTCCCCCACTGTTTGGCTTTCAGCTATATGGATGATGTG
GTATTGGGGGCCAAGTCTGTACAACATCTTGAGTCCCTTTTTACCTCTAT
TACCAATTTTCTTTTGTCTTTGGGTATACATTTGAACCCTAATAAAACCA
AACGTTGGGGCTACTCCCTTAACTTCATGGGATATGTAATTGGAAGTTGG
GGTACTTTACCGCAGGAACATATTGTACAAAAACTCAAGCAATGTTTTCG
AAAATTGCCTGTAAATAGACCTATTGATTGGAAAGTATGTCAAAGAATTG
TGGGTCTTTTGGGCTTTGCTGCCCCTTTTACACAATGTGGCTATCCTGCC
TTGATGCCTTTATATGCATGTATACAATCTAAGCAGGCTTTCACTTTCTC
GCCAACTTACAAGGCCTTTCTGTGTAAACAATATCTAAACCTTTACCCCG
TTGCCCGGCAACGGTCAGGTCTCTGCCAAGTGTTTGCTGACGCAACCCCC
ACGGGTTGGGGCTTGGCCATAGGCCATCGGCGCATGCGTGGAACCTTTGT
GGCTCCTCTGCCGATCCATACTGCGGAACTCCTAGCAGCTTGTTTTGCTC
GCAGCCGGTCTGGAGCGAAACTTATCGGAACCGACAACTCAGTTGTCCTC
TCTCGGAAATACACCTCCTTTCCATGGCTGCTAGGCTGTGCTGCCAACTG
GATCCTGCGCGGGACGTCCTTTGTCTACGTCCCGTCGGCGCTGAATCCCG
CGGACGACCCGTCTCGGGGCCGTTTGGGCCTCTACCGTCCCCTTCTTCAT
CTGCCGTTCCGGCCGACCACGGGGCGCACCTCTCTTTACGCGGTCTCCCC
GTCTGTGCCTTCTCATCTGCCGGACCGTGTGCACTTCGCTTCACCTCTGC
ACGTAGCATGGAGACCACCGTGAACGCCCACCAGGTCTTGCCCAAGGTCT
TACACAAGAGGACTCTTGGACTCTCAGCAATGTCAACGACCGACCTTGAG
GCATACTTCAAAGACTGTTTGTTTAAAGACTGGGAGGAGTTGGGGGAGGA
GATTAGGTTAAAGGTCTTTGTACTAGGAGGCTGTAGGCATAAATTGGTCT
GTTCACCAGCACCATGCAACTTTTTCCCCTCTGCCTAATCATCTCATGTT
CATGTCCTACTGTTCAAGCCTCCAAGCTGTGCCTTGGGTGGCTTTGGGGC
ATGGACATTGACCCGTATAAAGAATTTGGAGCTTCTGTGGAGTTACTCTC
TTTTTTGCCTTCTGACTTCTTTCCTTCTATTCGAGATCTCCTCGACACCG
CCTCTGCTCTGTATCGGGAGGCCTTAGAGTCTCCGGAACATTGTTCACCT
CACCATACAGCACTCAGGCAAGCTATTCTGTGTTGGGGTGAGTTGATGAA
TCTGGCCACCTGGGTGGGAAGTAATTTGGAAGACCCAGCATCCAGGGAAT
TAGTAGTCAGCTATGTCAATGTTAATATGGGCCTAAAAATTAGACAACTA
TTGTGGTTTCACATTTCCTGCCTTACTTTTGGAAGAGAAACTGTCCTTGA
GTATTTGGTGTCTTTTGGAGTGTGGATTCGCACTCCTCCCGCTTACAGAC
CACCAAATGCCCCTATCTTATCAACACTTCCGGAAACTACTGTTGTTAGA
CGACGAGGCAGGTCCCCTAGAAGAAGAACTCCCTCGCCTCGCAGACGAAG
GTCTCAATCGCCGCGTCGCAGAAGATCTCAATCTCGGGAATCTCAATGTT
AGTATCCCTTGGACTCATAAGGTGGGAAACTTTACTGGGCTTTATTCTTC
TACTGTACCTGTCTTTAATCCTGATTGGAAAACTCCCTCCTTTCCTCACA
TTCATTTACAGGAGGACATTATTAATAGATGTCAACAATATGTGGGCCCT
CTGACAGTTAATGAAAAAAGGAGATTAAAATTAATTATGCCTGCTAGGTT
CTATCCTAACCTTACCAAATATTTGCCCTTGGACAAAGGCATTAAACCGT
ATTATCCTGAATATGCAGTTAATCATTACTTCAAAACTAGGCATTATTTA
CATACTCTGTGGAAGGCTGGCATTCTATATAAGAGAGAAACTACACGCAG
CGCCTCATTTTGTGGGTCACCATATTCTTGGGAACAAGAGCTACAGCATG
GGAGGTTGGTCTTCCAAACCTCGACAAGGCATGGGGACGAATCTTTCTGT
TCCCAATCCTCTGGGATTCTTTCCCGATCACCAGTTGGACCCTGCGTTCG
GAGCCAACTCAAACAATCCAGATTGGGACTTCAACCCCAACAAGGATCAC
TGGCCAGAGGCAAATCAGGTAGGAGCGGGAGCATTTGGTCCAGGGTTCAC
CCCACCACACGGAGGCCTTTTGGGGTGGAGCCCTCAGGCTCAGGGCATAT
TGACAACACTGCCAGCAGCACCTCCTCCTGCCTCCACCAATCGGCAGTCA
GGAAGACAGCCTACTCCCATCTCTCCACCTCTAAGAGACAGTCATCCTCA
GGCCATGCAGTGGAA

PARP1

Poly [ADP-ribose] polymerase 1 (PARP-1) is an enzyme that in humans is encoded by the PARP1 gene. PARP1 works to on single strands of DNA, modifies nuclear proteins by poly ADP-ribosylation, involved in differentiation, proliferation and tumor transformation. PARP1 also has a role in repair of single-stranded DNA (ssDNA) breaks. Reducing intracellular PARP1 levels with siRNA or inhibiting PARP1 activity with small molecules reduces repair of ssDNA breaks. In the absence of PARP1, when these breaks are encountered during DNA replication, the replication fork stalls, and double-strand DNA (dsDNA) breaks accumulate. These dsDNA breaks are repaired via homologous recombination (HR) repair, a potentially error-free repair mechanism. However, both BRCA1 and BRCA2 are at least partially necessary for the HR pathway to function. Therefore, cells that are deficient in BRCA1 or BRCA2 have been shown to be highly sensitive to PARP1 inhibition or knock-down, resulting in cell death by apoptosis, in stark contrast to cells with at least one good copy of both BRCA1 and BRCA2. Many breast cancers have defects in the BRCA1/BRCA2 HR repair pathway due to mutations in either BRCA1 or BRCA2 (termed BRCAness), or other essential genes in the pathway and thus thought to be highly sensitive to PARP1 inhibitors. PARP1 inhibitors are believed to be effective for cancers with BRCAness, due to the high sensitivity of the tumors to the inhibitor and the lack of deleterious effects on the remaining healthy cells with functioning BRCA HR pathway (Bryant et al. (2005) Nature 434 (7035): 913-7 and Farmer et al. (2005) Nature 434 (7035): 917-21. This is in contrast to conventional chemotherapies, which are highly toxic to all cells and can induce DNA damage in healthy cells, leading to secondary cancer generation.

Protein: PARP1 Gene: PARP1: (Homo sapiens, chromosome 1, 226548392-226595801 [NCBI Reference Sequence NC_{—}000001.10]; start site location: 226595630; strand: negative)

Gene Identification

| | |
|---|---|
| GeneID | 142 |
| HGNC | 270 |
| MIM | 173870 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 9595 | | CCGCCAAAGCTCCGGAAGCCCGACGCC | 14 |
| 9741 | | CCGCCTCGCCGCCTCGCGTGCGCTC | 60 |
| 9887 | | CGGGAACGCCCACGGAACCCGCGTC | 177 |
| 9933 | | CGGGTGGAGCTCTGCGGGCCGCTGC | 269 |
| 9992 | | CGCCGGCCCCAAACTCTTAAGTGTG | 696 |
| 10014 | | CGGGAAGCGCAGGCCCCCGCCTCGG | 749 |
| 10045 | | CGTTCTAACCTGCCGTCCACAGACC | 839 |

Target Shift Sequences

| | | Relative |
|---|---|---|
| | | upstream |
| | | location |
| Sequence | | to gene |
| ID No: | Sequence (5′-3′) | start site |
| 9595 | CCGCCAAAGCTCCGGAAGCCCGACGCC | 14 |
| 9596 | CGCCAAAGCTCCGGAAGCCC | 15 |
| 9597 | GCCAAAGCTCCGGAAGCCCG | 16 |
| 9598 | CCAAAGCTCCGGAAGCCCGA | 17 |
| 9599 | CAAAGCTCCGGAAGCCCGAC | 18 |
| 9600 | AAAGCTCCGGAAGCCCGACG | 19 |
| 9601 | AAGCTCCGGAAGCCCGACGC | 20 |
| 9602 | AGCTCCGGAAGCCCGACGCC | 21 |
| 9603 | GCTCCGGAAGCCCGACGCCA | 22 |
| 9604 | CTCCGGAAGCCCGACGCCAC | 23 |
| 9605 | TCCGGAAGCCCGACGCCACG | 24 |
| 9606 | CCGGAAGCCCGACGCCACGA | 25 |
| 9607 | CGGAAGCCCGACGCCACGAC | 26 |
| 9608 | GGAAGCCCGACGCCACGACC | 27 |
| 9609 | GAAGCCCGACGCCACGACCT | 28 |
| 9610 | AAGCCCGACGCCACGACCTA | 29 |
| 9611 | AGCCCGACGCCACGACCTAG | 30 |
| 9612 | GCCCGACGCCACGACCTAGA | 31 |
| 9613 | CCCGACGCCACGACCTAGAA | 32 |
| 9614 | CCGACGCCACGACCTAGAAA | 33 |
| 9615 | CGACGCCACGACCTAGAAAC | 34 |
| 9616 | GACGCCACGACCTAGAAACA | 35 |
| 9617 | ACGCCACGACCTAGAAACAC | 36 |
| 9618 | CGCCACGACCTAGAAACACG | 37 |
| 9619 | GCCACGACCTAGAAACACGC | 38 |
| 9620 | CCACGACCTAGAAACACGCT | 39 |
| 9621 | CACGACCTAGAAACACGCTG | 40 |
| 9622 | ACGACCTAGAAACACGCTGC | 41 |
| 9623 | CGACCTAGAAACACGCTGCC | 42 |
| 9624 | GACCTAGAAACACGCTGCCG | 43 |
| 9625 | ACCTAGAAACACGCTGCCGC | 44 |
| 9626 | CCTAGAAACACGCTGCCGCC | 45 |
| 9627 | CTAGAAACACGCTGCCGCCT | 46 |
| 9628 | TAGAAACACGCTGCCGCCTC | 47 |
| 9629 | AGAAACACGCTGCCGCCTCG | 48 |
| 9630 | GAAACACGCTGCCGCCTCGC | 49 |
| 9631 | AAACACGCTGCCGCCTCGCC | 50 |
| 9632 | AACACGCTGCCGCCTCGCCG | 51 |
| 9633 | ACACGCTGCCGCCTCGCCGC | 52 |
| 9634 | CACGCTGCCGCCTCGCCGCC | 53 |
| 9635 | ACGCTGCCGCCTCGCCGCCT | 54 |
| 9636 | CGCTGCCGCCTCGCCGCCTC | 55 |
| 9637 | GCTGCCGCCTCGCCGCCTCG | 56 |
| 9638 | CTGCCGCCTCGCCGCCTCGC | 57 |
| 9639 | TGCCGCCTCGCCGCCTCGCG | 58 |
| 9640 | GCCGCCTCGCCGCCTCGCGT | 59 |
| 9641 | CCGCCTCGCCGCCTCGCGTG | 60 |
| 9642 | CGCCTCGCCGCCTCGCGTGC | 61 |
| 9643 | GCCTCGCCGCCTCGCGTGCG | 62 |
| 9644 | CCTCGCCGCCTCGCGTGCGC | 63 |
| 9645 | CTCGCCGCCTCGCGTGCGCT | 64 |
| 9646 | TCGCCGCCTCGCGTGCGCTC | 65 |
| 9647 | CGCCGCCTCGCGTGCGCTCA | 66 |
| 9648 | GCCGCCTCGCGTGCGCTCAC | 67 |
| 9649 | CCGCCTCGCGTGCGCTCACC | 68 |
| 9650 | CGCCTCGCGTGCGCTCACCC | 69 |
| 9651 | GCCTCGCGTGCGCTCACCCA | 70 |
| 9652 | CCTCGCGTGCGCTCACCCAG | 71 |
| 9653 | CTCGCGTGCGCTCACCCAGC | 72 |
| 9654 | TCGCGTGCGCTCACCCAGCC | 73 |
| 9655 | CGCGTGCGCTCACCCAGCCG | 74 |
| 9656 | GCGTGCGCTCACCCAGCCGC | 75 |
| 9657 | CGTGCGCTCACCCAGCCGCA | 76 |
| 9658 | GTGCGCTCACCCAGCCGCAG | 77 |
| 9659 | TGCGCTCACCCAGCCGCAGG | 78 |
| 9660 | GCGCTCACCCAGCCGCAGGC | 79 |
| 9661 | CGCTCACCCAGCCGCAGGCG | 80 |
| 9662 | GCTCACCCAGCCGCAGGCGC | 81 |
| 9663 | CTCACCCAGCCGCAGGCGCC | 82 |
| 9664 | TCACCCAGCCGCAGGCGCCT | 83 |
| 9665 | CACCCAGCCGCAGGCGCCTG | 84 |
| 9666 | ACCCAGCCGCAGGCGCCTGA | 85 |
| 9667 | CCCAGCCGCAGGCGCCTGAG | 86 |
| 9668 | CCAGCCGCAGGCGCCTGAGC | 87 |
| 9669 | CAGCCGCAGGCGCCTGAGCG | 88 |
| 9670 | AGCCGCAGGCGCCTGAGCGG | 89 |
| 9671 | GCCGCAGGCGCCTGAGCGGC | 90 |
| 9672 | CCGCAGGCGCCTGAGCGGCC | 91 |
| 9673 | CGCAGGCGCCTGAGCGGCCA | 92 |
| 9674 | GCAGGCGCCTGAGCGGCCAG | 93 |
| 9675 | CAGGCGCCTGAGCGGCCAGA | 94 |
| 9676 | AGGCGCCTGAGCGGCCAGAG | 95 |
| 9677 | GGCGCCTGAGCGGCCAGAGC | 96 |
| 9678 | GCGCCTGAGCGGCCAGAGCC | 97 |
| 9679 | CGCCTGAGCGGCCAGAGCCG | 98 |
| 9680 | GCCTGAGCGGCCAGAGCCGC | 99 |
| 9681 | CCTGAGCGGCCAGAGCCGCC | 100 |
| 9682 | CTGAGCGGCCAGAGCCGCCA | 101 |
| 9683 | TGAGCGGCCAGAGCCGCCAC | 102 |
| 9684 | GAGCGGCCAGAGCCGCCACC | 103 |
| 9685 | AGCGGCCAGAGCCGCCACCG | 104 |
| 9686 | GCGGCCAGAGCCGCCACCGA | 105 |
| 9687 | CGGCCAGAGCCGCCACCGAA | 106 |
| 9688 | GGCCAGAGCCGCCACCGAAC | 107 |
| 9689 | GCCAGAGCCGCCACCGAACA | 108 |
| 9690 | CCAGAGCCGCCACCGAACAC | 109 |
| 9691 | CAGAGCCGCCACCGAACACG | 110 |
| 9692 | AGAGCCGCCACCGAACACGC | 111 |
| 9693 | GAGCCGCCACCGAACACGCC | 112 |
| 9694 | AGCCGCCACCGAACACGCCG | 113 |
| 9695 | GCCGCCACCGAACACGCCGC | 114 |
| 9696 | CCGCCACCGAACACGCCGCA | 115 |
| 9697 | CGCCACCGAACACGCCGCAC | 116 |
| 9698 | GCCACCGAACACGCCGCACC | 117 |
| 9699 | CCACCGAACACGCCGCACCG | 118 |
| 9700 | CACCGAACACGCCGCACCGG | 119 |
| 9701 | ACCGAACACGCCGCACCGGC | 120 |
| 9702 | CCGAACACGCCGCACCGGCC | 121 |
| 9703 | CGAACACGCCGCACCGGCCA | 122 |
| 9704 | GAACACGCCGCACCGGCCAC | 123 |
| 9705 | AACACGCCGCACCGGCCACC | 124 |
| 9706 | ACACGCCGCACCGGCCACCG | 125 |
| 9707 | CACGCCGCACCGGCCACCGC | 126 |
| 9708 | ACGCCGCACCGGCCACCGCC | 127 |
| 9709 | CGCCGCACCGGCCACCGCCG | 128 |
| 9710 | GCCGCACCGGCCACCGCCGT | 129 |
| 9711 | CCGCACCGGCCACCGCCGTT | 130 |
| 9712 | CGCACCGGCCACCGCCGTTC | 131 |
| 9713 | GCACCGGCCACCGCCGTTCC | 132 |
| 9714 | CACCGGCCACCGCCGTTCCC | 133 |
| 9715 | ACCGGCCACCGCCGTTCCCT | 134 |
| 9716 | CCGGCCACCGCCGTTCCCTG | 135 |
| 9717 | CGGCCACCGCCGTTCCCTGA | 136 |
| 9718 | GGCCACCGCCGTTCCCTGAT | 137 |
| 9719 | GCCACCGCCGTTCCCTGATA | 138 |
| 9720 | CCACCGCCGTTCCCTGATAG | 139 |
| 9721 | CACCGCCGTTCCCTGATAGA | 140 |
| 9722 | ACCGCCGTTCCCTGATAGAT | 141 |
| 9723 | CCGCCGTTCCCTGATAGATT | 142 |
| 9724 | CGCCGTTCCCTGATAGATTG | 143 |
| 9725 | GCCGTTCCCTGATAGATTGC | 144 |
| 9726 | CCGTTCCCTGATAGATTGCT | 145 |
| 9727 | CGTTCCCTGATAGATTGCTG | 146 |
| 9728 | GCCGCCAAAGCTCCGGAAGC | 13 |
| 9729 | TGCCGCCAAAGCTCCGGAAG | 12 |
| 9730 | CTGCCGCCAAAGCTCCGGAA | 11 |
| 9731 | GCTGCCGCCAAAGCTCCGGA | 10 |
| 9732 | AGCTGCCGCCAAAGCTCCGG | 9 |
| 9733 | TAGCTGCCGCCAAAGCTCCG | 8 |
| 9734 | CTAGCTGCCGCCAAAGCTCC | 7 |
| 9735 | CCTAGCTGCCGCCAAAGCTC | 6 |
| 9736 | CCCTAGCTGCCGCCAAAGCT | 5 |
| 9737 | CCCCTAGCTGCCGCCAAAGC | 4 |
| 9738 | TCCCCTAGCTGCCGCCAAAG | 3 |
| 9739 | CTCCCCTAGCTGCCGCCAAA | 2 |
| 9740 | CCTCCCCTAGCTGCCGCCAA | 1 |
| 9741 | CCGCCTCGCCGCCTCGCGTGCGCTC | 60 |
| 9742 | CGCCTCGCCGCCTCGCGTGC | 61 |
| 9743 | GCCTCGCCGCCTCGCGTGCG | 62 |
| 9744 | CCTCGCCGCCTCGCGTGCGC | 63 |
| 9745 | CTCGCCGCCTCGCGTGCGCT | 64 |
| 9746 | TCGCCGCCTCGCGTGCGCTC | 65 |
| 9747 | CGCCGCCTCGCGTGCGCTCA | 66 |
| 9748 | GCCGCCTCGCGTGCGCTCAC | 67 |
| 9749 | CCGCCTCGCGTGCGCTCACC | 68 |
| 9750 | CGCCTCGCGTGCGCTCACCC | 69 |
| 9751 | GCCTCGCGTGCGCTCACCCA | 70 |
| 9752 | CCTCGCGTGCGCTCACCCAG | 71 |
| 9753 | CTCGCGTGCGCTCACCCAGC | 72 |
| 9754 | TCGCGTGCGCTCACCCAGCC | 73 |
| 9755 | CGCGTGCGCTCACCCAGCCG | 74 |
| 9756 | GCGTGCGCTCACCCAGCCGC | 75 |
| 9757 | CGTGCGCTCACCCAGCCGCA | 76 |
| 9758 | GTGCGCTCACCCAGCCGCAG | 77 |
| 9759 | TGCGCTCACCCAGCCGCAGG | 78 |
| 9760 | GCGCTCACCCAGCCGCAGGC | 79 |
| 9761 | CGCTCACCCAGCCGCAGGCG | 80 |
| 9762 | GCTCACCCAGCCGCAGGCGC | 81 |
| 9763 | CTCACCCAGCCGCAGGCGCC | 82 |
| 9764 | TCACCCAGCCGCAGGCGCCT | 83 |
| 9765 | CACCCAGCCGCAGGCGCCTG | 84 |
| 9766 | ACCCAGCCGCAGGCGCCTGA | 85 |
| 9767 | CCCAGCCGCAGGCGCCTGAG | 86 |
| 9768 | CCAGCCGCAGGCGCCTGAGC | 87 |
| 9769 | CAGCCGCAGGCGCCTGAGCG | 88 |
| 9770 | AGCCGCAGGCGCCTGAGCGG | 89 |
| 9771 | GCCGCAGGCGCCTGAGCGGC | 90 |
| 9772 | CCGCAGGCGCCTGAGCGGCC | 91 |
| 9773 | CGCAGGCGCCTGAGCGGCCA | 92 |
| 9774 | GCAGGCGCCTGAGCGGCCAG | 93 |
| 9775 | CAGGCGCCTGAGCGGCCAGA | 94 |
| 9776 | AGGCGCCTGAGCGGCCAGAG | 95 |
| 9777 | GGCGCCTGAGCGGCCAGAGC | 96 |
| 9778 | GCGCCTGAGCGGCCAGAGCC | 97 |
| 9779 | CGCCTGAGCGGCCAGAGCCG | 98 |
| 9780 | GCCTGAGCGGCCAGAGCCGC | 99 |
| 9781 | CCTGAGCGGCCAGAGCCGCC | 100 |
| 9782 | CTGAGCGGCCAGAGCCGCCA | 101 |
| 9783 | TGAGCGGCCAGAGCCGCCAC | 102 |
| 9784 | GAGCGGCCAGAGCCGCCACC | 103 |
| 9785 | AGCGGCCAGAGCCGCCACCG | 104 |
| 9786 | GCGGCCAGAGCCGCCACCGA | 105 |
| 9787 | CGGCCAGAGCCGCCACCGAA | 106 |
| 9788 | GGCCAGAGCCGCCACCGAAC | 107 |
| 9789 | GCCAGAGCCGCCACCGAACA | 108 |
| 9790 | CCAGAGCCGCCACCGAACAC | 109 |
| 9791 | CAGAGCCGCCACCGAACACG | 110 |
| 9792 | AGAGCCGCCACCGAACACGC | 111 |
| 9793 | GAGCCGCCACCGAACACGCC | 112 |
| 9794 | AGCCGCCACCGAACACGCCG | 113 |
| 9795 | GCCGCCACCGAACACGCCGC | 114 |
| 9796 | CCGCCACCGAACACGCCGCA | 115 |
| 9797 | CGCCACCGAACACGCCGCAC | 116 |
| 9798 | GCCACCGAACACGCCGCACC | 117 |
| 9799 | CCACCGAACACGCCGCACCG | 118 |
| 9800 | CACCGAACACGCCGCACCGG | 119 |
| 9801 | ACCGAACACGCCGCACCGGC | 120 |
| 9802 | CCGAACACGCCGCACCGGCC | 121 |
| 9803 | CGAACACGCCGCACCGGCCA | 122 |
| 9804 | GAACACGCCGCACCGGCCAC | 123 |
| 9805 | AACACGCCGCACCGGCCACC | 124 |
| 9806 | ACACGCCGCACCGGCCACCG | 125 |
| 9807 | CACGCCGCACCGGCCACCGC | 126 |
| 9808 | ACGCCGCACCGGCCACCGCC | 127 |
| 9809 | CGCCGCACCGGCCACCGCCG | 128 |
| 9810 | GCCGCACCGGCCACCGCCGT | 129 |
| 9811 | CCGCACCGGCCACCGCCGTT | 130 |
| 9812 | CGCACCGGCCACCGCCGTTC | 131 |
| 9813 | GCACCGGCCACCGCCGTTCC | 132 |
| 9814 | CACCGGCCACCGCCGTTCCC | 133 |
| 9815 | ACCGGCCACCGCCGTTCCCT | 134 |
| 9816 | CCGGCCACCGCCGTTCCCTG | 135 |
| 9817 | CGGCCACCGCCGTTCCCTGA | 136 |
| 9818 | GGCCACCGCCGTTCCCTGAT | 137 |
| 9819 | GCCACCGCCGTTCCCTGATA | 138 |
| 9820 | CCACCGCCGTTCCCTGATAG | 139 |
| 9821 | CACCGCCGTTCCCTGATAGA | 140 |
| 9822 | ACCGCCGTTCCCTGATAGAT | 141 |
| 9823 | CCGCCGTTCCCTGATAGATT | 142 |
| 9824 | CGCCGTTCCCTGATAGATTG | 143 |
| 9825 | GCCGTTCCCTGATAGATTGC | 144 |
| 9826 | CCGTTCCCTGATAGATTGCT | 145 |
| 9827 | CGTTCCCTGATAGATTGCTG | 146 |
| 9828 | GCCGCCTCGCCGCCTCGCGT | 59 |
| 9829 | TGCCGCCTCGCCGCCTCGCG | 58 |
| 9830 | CTGCCGCCTCGCCGCCTCGC | 57 |
| 9831 | GCTGCCGCCTCGCCGCCTCG | 56 |
| 9832 | CGCTGCCGCCTCGCCGCCTC | 55 |
| 9833 | ACGCTGCCGCCTCGCCGCCT | 54 |
| 9834 | CACGCTGCCGCCTCGCCGCC | 53 |
| 9835 | ACACGCTGCCGCCTCGCCGC | 52 |
| 9836 | AACACGCTGCCGCCTCGCCG | 51 |
| 9837 | AAACACGCTGCCGCCTCGCC | 50 |
| 9838 | GAAACACGCTGCCGCCTCGC | 49 |
| 9839 | AGAAACACGCTGCCGCCTCG | 48 |
| 9840 | TAGAAACACGCTGCCGCCTC | 47 |
| 9841 | CTAGAAACACGCTGCCGCCT | 46 |
| 9842 | CCTAGAAACACGCTGCCGCC | 45 |
| 9843 | ACCTAGAAACACGCTGCCGC | 44 |
| 9844 | GACCTAGAAACACGCTGCCG | 43 |
| 9845 | CGACCTAGAAACACGCTGCC | 42 |
| 9846 | ACGACCTAGAAACACGCTGC | 41 |
| 9847 | CACGACCTAGAAACACGCTG | 40 |
| 9848 | CCACGACCTAGAAACACGCT | 39 |
| 9849 | GCCACGACCTAGAAACACGC | 38 |
| 9850 | CGCCACGACCTAGAAACACG | 37 |
| 9851 | ACGCCACGACCTAGAAACAC | 36 |
| 9852 | GACGCCACGACCTAGAAACA | 35 |
| 9853 | CGACGCCACGACCTAGAAAC | 34 |
| 9854 | CCGACGCCACGACCTAGAAA | 33 |
| 9855 | CCCGACGCCACGACCTAGAA | 32 |
| 9856 | GCCCGACGCCACGACCTAGA | 31 |
| 9857 | AGCCCGACGCCACGACCTAG | 30 |
| 9858 | AAGCCCGACGCCACGACCTA | 29 |
| 9859 | GAAGCCCGACGCCACGACCT | 28 |
| 9860 | GGAAGCCCGACGCCACGACC | 27 |
| 9861 | CGGAAGCCCGACGCCACGAC | 26 |
| 9862 | CCGGAAGCCCGACGCCACGA | 25 |
| 9863 | TCCGGAAGCCCGACGCCACG | 24 |
| 9864 | CTCCGGAAGCCCGACGCCAC | 23 |
| 9865 | GCTCCGGAAGCCCGACGCCA | 22 |
| 9866 | AGCTCCGGAAGCCCGACGCC | 21 |
| 9867 | AAGCTCCGGAAGCCCGACGC | 20 |
| 9868 | AAAGCTCCGGAAGCCCGACG | 19 |
| 9869 | CAAAGCTCCGGAAGCCCGAC | 18 |
| 9870 | CCAAAGCTCCGGAAGCCCGA | 17 |
| 9871 | GCCAAAGCTCCGGAAGCCCG | 16 |
| 9872 | CGCCAAAGCTCCGGAAGCCC | 15 |
| 9873 | CCGCCAAAGCTCCGGAAGCC | 14 |
| 9874 | GCCGCCAAAGCTCCGGAAGC | 13 |
| 9875 | TGCCGCCAAAGCTCCGGAAG | 12 |
| 9876 | CTGCCGCCAAAGCTCCGGAA | 11 |
| 9877 | GCTGCCGCCAAAGCTCCGGA | 10 |
| 9878 | AGCTGCCGCCAAAGCTCCGG | 9 |
| 9879 | TAGCTGCCGCCAAAGCTCCG | 8 |
| 9880 | CTAGCTGCCGCCAAAGCTCC | 7 |
| 9881 | CCTAGCTGCCGCCAAAGCTC | 6 |
| 9882 | CCCTAGCTGCCGCCAAAGCT | 5 |
| 9883 | CCCCTAGCTGCCGCCAAAGC | 4 |
| 9884 | TCCCCTAGCTGCCGCCAAAG | 3 |
| 9885 | CTCCCCTAGCTGCCGCCAAA | 2 |
| 9886 | CCTCCCCTAGCTGCCGCCAA | 1 |
| 9887 | CGGGAACGCCCACGGAACCCGCGTC | 177 |
| 9888 | GGGAACGCCCACGGAACCCG | 178 |
| 9889 | GGAACGCCCACGGAACCCGC | 179 |
| 9890 | GAACGCCCACGGAACCCGCG | 180 |
| 9891 | AACGCCCACGGAACCCGCGT | 181 |
| 9892 | ACGCCCACGGAACCCGCGTC | 182 |
| 9893 | CGCCCACGGAACCCGCGTCC | 183 |
| 9894 | GCCCACGGAACCCGCGTCCA | 184 |
| 9895 | CCCACGGAACCCGCGTCCAC | 185 |
| 9896 | CCACGGAACCCGCGTCCACG | 186 |
| 9897 | CACGGAACCCGCGTCCACGG | 187 |
| 9898 | ACGGAACCCGCGTCCACGGG | 188 |
| 9899 | CGGAACCCGCGTCCACGGGG | 189 |
| 9900 | GGAACCCGCGTCCACGGGGC | 190 |
| 9901 | GAACCCGCGTCCACGGGGCG | 191 |
| 9902 | AACCCGCGTCCACGGGGCGG | 192 |
| 9903 | ACCCGCGTCCACGGGGCGGG | 193 |
| 9904 | CCCGCGTCCACGGGGCGGGG | 194 |
| 9905 | CCGCGTCCACGGGGCGGGGC | 195 |
| 9906 | CGCGTCCACGGGGCGGGGCC | 196 |
| 9907 | GCGTCCACGGGGCGGGGCCG | 197 |
| 9908 | CGTCCACGGGGCGGGGCCGG | 198 |
| 9909 | GTCCACGGGGCGGGGCCGGC | 199 |
| 9910 | TCCACGGGGCGGGGCCGGCG | 200 |
| 9911 | CCACGGGGCGGGGCCGGCGG | 201 |
| 9912 | CACGGGGCGGGGCCGGCGGC | 202 |
| 9913 | GCGGGAACGCCCACGGAACC | 176 |
| 9914 | CGCGGGAACGCCCACGGAAC | 175 |
| 9915 | CCGCGGGAACGCCCACGGAA | 174 |
| 9916 | GCCGCGGGAACGCCCACGGA | 173 |
| 9917 | GGCCGCGGGAACGCCCACGG | 172 |
| 9918 | TGGCCGCGGGAACGCCCACG | 171 |
| 9919 | CTGGCCGCGGGAACGCCCAC | 170 |
| 9920 | CCTGGCCGCGGGAACGCCCA | 169 |
| 9921 | GCCTGGCCGCGGGAACGCCC | 168 |
| 9922 | TGCCTGGCCGCGGGAACGCC | 167 |
| 9923 | ATGCCTGGCCGCGGGAACGC | 166 |
| 9924 | GATGCCTGGCCGCGGGAACG | 165 |
| 9925 | TGATGCCTGGCCGCGGGAAC | 164 |
| 9926 | CTGATGCCTGGCCGCGGGAA | 163 |
| 9927 | GCTGATGCCTGGCCGCGGGA | 162 |
| 9928 | TGCTGATGCCTGGCCGCGGG | 161 |
| 9929 | TTGCTGATGCCTGGCCGCGG | 160 |
| 9930 | ATTGCTGATGCCTGGCCGCG | 159 |
| 9931 | GATTGCTGATGCCTGGCCGC | 158 |
| 9932 | AGATTGCTGATGCCTGGCCG | 157 |
| 9933 | CGGGTGGAGCTCTGCGGGCCGCTGC | 269 |
| 9934 | GGGTGGAGCTCTGCGGGCCG | 270 |
| 9935 | GGTGGAGCTCTGCGGGCCGC | 271 |
| 9936 | GTGGAGCTCTGCGGGCCGCT | 272 |
| 9937 | TGGAGCTCTGCGGGCCGCTG | 273 |
| 9938 | GGAGCTCTGCGGGCCGCTGC | 274 |
| 9939 | GAGCTCTGCGGGCCGCTGCC | 275 |
| 9940 | AGCTCTGCGGGCCGCTGCCC | 276 |
| 9941 | GCTCTGCGGGCCGCTGCCCT | 277 |
| 9942 | CTCTGCGGGCCGCTGCCCTG | 278 |
| 9943 | TCTGCGGGCCGCTGCCCTGG | 279 |
| 9944 | CTGCGGGCCGCTGCCCTGGG | 280 |
| 9945 | TGCGGGCCGCTGCCCTGGGG | 281 |
| 9946 | GCGGGCCGCTGCCCTGGGGG | 282 |
| 9947 | CGGGCCGCTGCCCTGGGGGC | 283 |
| 9948 | GGGCCGCTGCCCTGGGGGCC | 284 |
| 9949 | GGCCGCTGCCCTGGGGGCCG | 285 |
| 9950 | GCCGCTGCCCTGGGGGCCGA | 286 |
| 9951 | CCGCTGCCCTGGGGGCCGAG | 287 |
| 9952 | CGCTGCCCTGGGGGCCGAGG | 288 |
| 9953 | GCTGCCCTGGGGGCCGAGGC | 289 |
| 9954 | CTGCCCTGGGGGCCGAGGCG | 290 |
| 9955 | TGCCCTGGGGGCCGAGGCGG | 291 |
| 9956 | GCCCTGGGGGCCGAGGCGGG | 292 |
| 9957 | CCCTGGGGGCCGAGGCGGGG | 293 |
| 9958 | CCTGGGGGCCGAGGCGGGGC | 294 |
| 9959 | CTGGGGGCCGAGGCGGGGCT | 295 |
| 9960 | TGGGGGCCGAGGCGGGGCTT | 296 |
| 9961 | CCGGGTGGAGCTCTGCGGGC | 268 |
| 9962 | GCCGGGTGGAGCTCTGCGGG | 267 |
| 9963 | TGCCGGGTGGAGCTCTGCGG | 266 |
| 9964 | CTGCCGGGTGGAGCTCTGCG | 265 |
| 9965 | CCTGCCGGGTGGAGCTCTGC | 264 |
| 9966 | GCCTGCCGGGTGGAGCTCTG | 263 |
| 9967 | CGCCTGCCGGGTGGAGCTCT | 262 |
| 9968 | GCGCCTGCCGGGTGGAGCTC | 261 |
| 9969 | GGCGCCTGCCGGGTGGAGCT | 260 |
| 9970 | GGGCGCCTGCCGGGTGGAGC | 259 |
| 9971 | CGGGCGCCTGCCGGGTGGAG | 258 |
| 9972 | CCGGGCGCCTGCCGGGTGGA | 257 |
| 9973 | CCCGGGCGCCTGCCGGGTGG | 256 |
| 9974 | TCCCGGGCGCCTGCCGGGTG | 255 |
| 9975 | TTCCCGGGCGCCTGCCGGGT | 254 |
| 9976 | TTTCCCGGGCGCCTGCCGGG | 253 |
| 9977 | GTTTCCCGGGCGCCTGCCGG | 252 |
| 9978 | AGTTTCCCGGGCGCCTGCCG | 251 |
| 9979 | GAGTTTCCCGGGCGCCTGCC | 250 |
| 9980 | GGAGTTTCCCGGGCGCCTGC | 249 |
| 9981 | CGGAGTTTCCCGGGCGCCTG | 248 |
| 9982 | GCGGAGTTTCCCGGGCGCCT | 247 |
| 9983 | GGCGGAGTTTCCCGGGCGCC | 246 |
| 9984 | GGGCGGAGTTTCCCGGGCGC | 245 |
| 9985 | GGGGCGGAGTTTCCCGGGCG | 244 |
| 9986 | GGGGGCGGAGTTTCCCGGGC | 243 |
| 9987 | GGGGGGCGGAGTTTCCCGGG | 242 |
| 9988 | CGGGGGGCGGAGTTTCCCGG | 241 |
| 9989 | CCGGGGGGCGGAGTTTCCCG | 240 |
| 9990 | GCCGGGGGGCGGAGTTTCCC | 239 |
| 9991 | GGCCGGGGGGCGGAGTTTCC | 238 |
| 9992 | CGCCGGCCCCAAACTCTTAAGTGTG | 696 |
| 9993 | GCCGGCCCCAAACTCTTAAG | 697 |
| 9994 | CCGGCCCCAAACTCTTAAGT | 698 |
| 9995 | CGGCCCCAAACTCTTAAGTG | 699 |
| 9996 | ACGCCGGCCCCAAACTCTTA | 695 |
| 9997 | CACGCCGGCCCCAAACTCTT | 694 |
| 9998 | CCACGCCGGCCCCAAACTCT | 693 |
| 9999 | ACCACGCCGGCCCCAAACTC | 692 |
| 10000 | TACCACGCCGGCCCCAAACT | 691 |
| 10001 | CTACCACGCCGGCCCCAAAC | 690 |
| 10002 | GCTACCACGCCGGCCCCAAA | 689 |
| 10003 | AGCTACCACGCCGGCCCCAA | 688 |
| 10004 | GAGCTACCACGCCGGCCCCA | 687 |
| 10005 | TGAGCTACCACGCCGGCCCC | 686 |
| 10006 | ATGAGCTACCACGCCGGCCC | 685 |
| 10007 | CATGAGCTACCACGCCGGCC | 684 |
| 10008 | GCATGAGCTACCACGCCGGC | 683 |
| 10009 | GGCATGAGCTACCACGCCGG | 682 |
| 10010 | GGGCATGAGCTACCACGCCG | 681 |
| 10011 | GGGGCATGAGCTACCACGCC | 680 |
| 10012 | AGGGGCATGAGCTACCACGC | 679 |
| 10013 | CAGGGGCATGAGCTACCACG | 678 |
| 10014 | CGGGAAGCGCAGGCCCCCGCCTCGG | 749 |
| 10015 | GGGAAGCGCAGGCCCCCGCC | 750 |
| 10016 | GGAAGCGCAGGCCCCCGCCT | 751 |
| 10017 | GAAGCGCAGGCCCCCGCCTC | 752 |
| 10018 | AAGCGCAGGCCCCCGCCTCG | 753 |
| 10019 | AGCGCAGGCCCCCGCCTCGG | 754 |
| 10020 | GCGCAGGCCCCCGCCTCGGG | 755 |
| 10021 | CGCAGGCCCCCGCCTCGGGA | 756 |
| 10022 | GCAGGCCCCCGCCTCGGGAA | 757 |
| 10023 | CAGGCCCCCGCCTCGGGAAT | 758 |
| 10024 | AGGCCCCCGCCTCGGGAATA | 759 |
| 10025 | GGCCCCCGCCTCGGGAATAT | 760 |
| 10026 | GCCCCCGCCTCGGGAATATA | 761 |
| 10027 | CCCCCGCCTCGGGAATATAG | 762 |
| 10028 | CCCCGCCTCGGGAATATAGT | 763 |
| 10029 | CCCGCCTCGGGAATATAGTT | 764 |
| 10030 | CCGCCTCGGGAATATAGTTG | 765 |
| 10031 | CGCCTCGGGAATATAGTTGA | 766 |
| 10032 | GCCTCGGGAATATAGTTGAT | 767 |
| 10033 | CCGGGAAGCGCAGGCCCCCG | 748 |
| 10034 | TCCGGGAAGCGCAGGCCCCC | 747 |
| 10035 | GTCCGGGAAGCGCAGGCCCC | 746 |
| 10036 | GGTCCGGGAAGCGCAGGCCC | 745 |
| 10037 | GGGTCCGGGAAGCGCAGGCC | 744 |
| 10038 | TGGGTCCGGGAAGCGCAGGC | 743 |
| 10039 | CTGGGTCCGGGAAGCGCAGG | 742 |
| 10040 | GCTGGGTCCGGGAAGCGCAG | 741 |
| 10041 | AGCTGGGTCCGGGAAGCGCA | 740 |
| 10042 | CAGCTGGGTCCGGGAAGCGC | 739 |
| 10043 | GCAGCTGGGTCCGGGAAGCG | 738 |
| 10044 | GGCAGCTGGGTCCGGGAAGC | 737 |
| 10045 | CGTTCTAACCTGCCGTCCACAGACC | 839 |
| 10046 | GTTCTAACCTGCCGTCCACA | 840 |
| 10047 | TTCTAACCTGCCGTCCACAG | 841 |
| 10048 | TCTAACCTGCCGTCCACAGA | 842 |
| 10049 | CTAACCTGCCGTCCACAGAC | 843 |
| 10050 | TAACCTGCCGTCCACAGACC | 844 |
| 10051 | AACCTGCCGTCCACAGACCG | 845 |
| 10052 | ACCTGCCGTCCACAGACCGT | 846 |
| 10053 | CCTGCCGTCCACAGACCGTC | 847 |
| 10054 | CTGCCGTCCACAGACCGTCG | 848 |
| 10055 | TGCCGTCCACAGACCGTCGG | 849 |
| 10056 | GCCGTCCACAGACCGTCGGG | 850 |
| 10057 | CCGTCCACAGACCGTCGGGA | 851 |
| 10058 | CGTCCACAGACCGTCGGGAC | 852 |
| 10059 | GTCCACAGACCGTCGGGACA | 853 |
| 10060 | TCCACAGACCGTCGGGACAA | 854 |
| 10061 | CCACAGACCGTCGGGACAAA | 855 |
| 10062 | CACAGACCGTCGGGACAAAA | 856 |
| 10063 | ACAGACCGTCGGGACAAAAT | 857 |
| 10064 | CAGACCGTCGGGACAAAATA | 858 |
| 10065 | AGACCGTCGGGACAAAATAC | 859 |
| 10066 | GACCGTCGGGACAAAATACC | 860 |
| 10067 | ACCGTCGGGACAAAATACCA | 861 |
| 10068 | CCGTCGGGACAAAATACCAA | 862 |
| 10069 | CGTCGGGACAAAATACCAAC | 863 |
| 10070 | GTCGGGACAAAATACCAACT | 864 |
| 10071 | TCGGGACAAAATACCAACTG | 865 |
| 10072 | CGGGACAAAATACCAACTGA | 866 |
| 10073 | GCGTTCTAACCTGCCGTCCA | 838 |
| 10074 | GGCGTTCTAACCTGCCGTCC | 837 |
| 10075 | GGGCGTTCTAACCTGCCGTC | 836 |
| 10076 | CGGGCGTTCTAACCTGCCGT | 835 |
| 10077 | ACGGGCGTTCTAACCTGCCG | 834 |
| 10078 | GACGGGCGTTCTAACCTGCC | 833 |
| 10079 | GGACGGGCGTTCTAACCTGC | 832 |
| 10080 | TGGACGGGCGTTCTAACCTG | 831 |
| 10081 | TTGGACGGGCGTTCTAACCT | 830 |
| 10082 | CTTGGACGGGCGTTCTAACC | 829 |
| 10083 | GCTTGGACGGGCGTTCTAAC | 828 |
| 10084 | GGCTTGGACGGGCGTTCTAA | 827 |
| 10085 | TGGCTTGGACGGGCGTTCTA | 826 |
| 10086 | CTGGCTTGGACGGGCGTTCT | 825 |
| 10087 | CCTGGCTTGGACGGGCGTTC | 824 |
| 10088 | TCCTGGCTTGGACGGGCGTT | 823 |
| 10089 | CTCCTGGCTTGGACGGGCGT | 822 |
| 10090 | CCTCCTGGCTTGGACGGGCG | 821 |
| 10091 | CCCTCCTGGCTTGGACGGGC | 820 |
| 10092 | ACCCTCCTGGCTTGGACGGG | 819 |
| 10093 | CACCCTCCTGGCTTGGACGG | 818 |
| 10094 | CCACCCTCCTGGCTTGGACG | 817 |

### Examples

Genetic Code (5′ Upstream Region)

(SEQ ID NO: 11981)

GGTGGATCTCCACATGCAGAAGAATGTAGCTGGACCCATACCTTACACCA
AATGTTTGTTGTGAGTTTATTTACTTTTTTGTGTGTGTGGAGACAGGGTC
GTGCTATGTTGTCCAGGCTGATCTAGAACTCCTTACCTAGAGACACTGCC
AAGGTAAGTGAGGGCCAAGTGGACACTGAGTGATTCTGTGCCTCACTGAG
CAAAAATAACTAAACATGGGCGAAGGAGAGCCCAATGATCCCAGGGACAA
AATGTCATCACGGGCATTCTGCGCACGCTTGCCAGGATACAGGAGAAGCA
ACCAGACACTTCATTCATCTTCTCAGAATGTTCATTAACATGTTCAGAAA
GGTGGAAAACCTTACTTGCTAAAGAGAAGGAAATTGGAGGCATGGCCAAA
AGTATTCAAGGCCCTTTATGAAAAAGAAATGAAAACTGATATCCCTCCTA
AAAGAGAAGTAAAACAGAAATTCAGAGATTCTAATGCACCCGAAAGGCCT
CCTTTGGGCTTTCACTTTGTGTTCTGAGTACTGCCCTCAAATCAAAGGAG
ATCCCGGTCTGTCCACTGGCAGTGATGCCAAGAACCTGGGAGGGACATGA
GTGACCATGCTGCAGATGGCAAGCAGCCCAAAAAGAAGGCTTCTCAACTG
AAGGAAAAGTACCAAGAGCAGAATGCTGCATATCCAGCCAAAGGAAAGCT
GATGTGGCAAAAATGATGCTGTCAAGGCCGAAAAAGGCAAGAAAAAAAAA
CAAAGCGGAGAAAGACAGAGAAGGTAAGGAAAATAAAAAATGAAGTCGAT
GATAATGACAAATAAGGTGGTTCTATGGCAGCTTTTTTTTTTTTCTCTTG
TCTATAAAGCATTTAACCTACCTGGACACAGCTCATTCCTTTTAAAGAAA
AAAATTGAAATGTAAAGCCACCTAAGATTTATTTGTAAACTGCATGATGG
CGTTCTTTTTCTGTTTTTGTATTATTAACAAGAATTATCAAGTAATTCTT
CAGACAACCCTGTCCTGGTGGTATTTTGTATAGCCACCAACTTTGCCTGG
TATACTATAGGGGTTATAAATCAGCATGGGAATTTCAAATTTAAGGCACA
GTATAAGTTAGTTATATACAAATGTGAAGTAACATTATTAATTAAACTGT
TGGCCTGTGCGAAGGGAGGGCCAACTGTGGGATTCAGTCATTCATTCAAC
AAATATTGGTGAGTGCCTGACACTGTTCCAGGCACTGAGGCTATTGCAAC
AAAACAGACACAAGCTCCTGCCCTCATGGAGCTTACATTCTGGTGAGGGA
TACAGAGCCACCAAAAAGGATGGCAGCTGGGCCATGAGAAAGGATCAAAG
TCAGGAAGTTAGAATTCGGGGATGGATTGAACATGGGACAAAAGAGAAGA
GTCAAGTTGACTACAAAGCATTTGGCCTAAGTAATGCAAAGAATGGTGGG
CCATTTCCTGAGATGGGAAGCACTAGGGTAGTTTTGGACATAAATGGAGA
TGCATATAAGCCATCCAAACTGAAATATTGAGAAGGCAGTAGGTGATAGT
TGGCTTTCCTTAGGTTCTAGGGCAGGAATTCTTAACCTTTTGTGTGTGTG
CCTAGGACCCCTTTGGTGGTCCATGAAGCCCTTTCCAGAATAAATATTGT
GGAGGAACCTACCTTAATGCAATAGTAGCTTCTAGGTACATTATCAGGCA
AACTATCCCACAAGTTACAAAACAGAAAGCCTCACAGACCAAATTATGAT
GCTTGAATTGCAGGGTTTATTGAATCAGTTTAAAACCACTTACAGCAAGA
ACTCGATGGGGTGCATAACATACACAGGATAGGGTACAGGCGAGGCAGAT
GGACCACACCACCAGAACCTAGAATTAGGGAATCCTCCCCTCCCCTCCCC
TCCCACCCCTCCCCCCTTCCCCCTCCCCTCCCCTCCCCTCCTCCCCTCCT
CTCCCATCCTCCCCTCCCCTCCCATCCTCCCCTCCCCTCCCCTTTTCTCT
TCTTTTCTTTTTTTGAGACTGTCTCACTATGTTGCCCAGGCTGGAGTGCA
ATGGCGTGATCTCGGCTCACTGCAACTTCCACCTCCTGGGTTCAAGCGAT
TCTCATGCCTCAGCCTCCCGAGTAGCTGGGATTACAGGCACGCACCACAA
TGCCCAGCTAATTTTTGTATTCTTAGTAGAGACGGGGTTTCACCATGTTG
ACCAGGCTGGTTTTGAACTCCTGACCTTAGGTGATCCACCCGCCTCAGAT
TCCCAAAGCGCTGGGATTACAGGCATGAGCCACTGCACCTGGCTAATATT
GATATGTTTTCCCTCTCTCTGCCGCATCAGCCTGTCCCACTGACAGAGTT
GAGGATGCTCAAGGCGGCTCAACAGAGGGTACCTGGAGCAACTCACACTG
CACTATCAGAGAGACACAAGTGCAAGCACACTCAGCCACAGCTGCAGCTC
ACCAATCAGCCTGCTGAACAGACCTGAACTTTAGCTGCATTTTTGGGGCA
GAGCATATGGGTGCCAGGATGGGACCATAATCTTATCACCAATGAGTGGC
CATTTAGGGATGATATAGTTGTCAACCCAGAGATGGCATGATCATGCCTT
TTGACTTGGTCATTCTCTAAGTAAAACTTTTATTTGTTCCATCATATTTT
CCACTTATTCTGTTTACCTTCAAAATATCTTTTTTTTTTTTTTTTGAGAC
AGGGTCACACTGTCACCCAGGCTAGAGTCCAGTGGCACTATCATGGCTCA
CCACAGCCTCAACCTTCAGGGCTCAGGTGATCCTCCCACTTCAGCCTCCC
GAGTAGATGGGACTACAGGCACCTGCCACCACCCCCAGCTAATTTTTGTA
GAGACAAGGTTTTGCCATGTTGTCCAGGCTGGTCTTGAACTCCTGGGCTC
AAGGGATCCGGCCACCTCAGCCTCCCAAAGTGCTAGGATTATAGGCATGA
GCCACTGTGCCCAGCCTACCTTCAACGTATCTAACTGGTTACTAACTTTT
AGGATTCGGCCTATGTCTCACAACCTTCTTGCTTACTCAACATCCTTGTC
TCTTAAGCCACTAGCTTCTTCTCTATGGTTAACACTTTTTATGAGTTTTA
TTCATCTGCTTATTTTTCTTATCCTCTATACCAGAATTGAATATTTTCAA
ATAAAGCACACTCATGTTACAATCTTTGAAATGAAAAAAAAAAATGCATA
GGATTAGAAAAGAAACCAATTTTAATAAACTATATTTTGAAGTATAGTTC
TATATTAAACAACAAGATCTAGGCCAGGTGCAGTGGCTCATGCCTGTAAT
CCCAGCAATTTGGGAAGTCGAGGTGGGAGGATTGCTTGAGGCCAGGGGTT
CAAGACCAGCCTGGGCAACATGGAGAGATTCCCCATCTCTTTCTTTACAC
ACACACACACACACACACACAAAATATCTGATAGCAACAGGTGCAGTCAT
TACCACAATTTCGAGTAGTGATGAGCTTAATAATATTTCGAGTTATCACC
AACAACTGTAAAGTAACATGAAAACGTCTGTGATGACTATTGCCCACAAA
GTCACAGGTACTGCTAATACTCCTGGTATTTGTAGTCAAATTCATAATAA
AGGAAATGCTAGGTTTCAGTTGGTATTTTGTCCCGACGGTCTGTGGACGG
CAGGTTAGAACGCCCGTCCAAGCCAGGAGGGTGGACCTAGCACTGCAGGG
TCCACCTCGGGCCAATCAACTATATTCCCGAGGCGGGGGCCTGCGCTTCC
CGGACCCAGCTGCCCTCAGGGGAGAGAGGACACACTTAAGAGTTTGGGGC
CGGCGTGGTAGCTCATGCCCCTGATCCCAGCACTTCGGGAGGCTGAGGCG
TGAAGATCACTTGTAGCAGGAGTTTGAGACCAGTCTAGCCAACTTGGCGA
GACCCTGTCCCTAAAAAAAATTTTTTTTTAATTAGCCAGTTGTGGTGAGC
GCCTGTAGTCCCAGCTACTCGGGAGGCTGAGGTGGGAGGATCGCTGGGCT
CAGGAGTTCCAGACTGCAGTGAGCCATGATGGCGGCACTGCACTCCAGCG
CGGTGAGACTCAGTCTCAAAAATAAAAGGGGGAGGGGTTGGGGGTAAAAT
TAGTTGTGAAATCAAGTAAGACTTCCTGGGACAGAACAATCAAAGGGGTG
GCGCCGGGTCCTCCAAAGAGCTACTAGCTCAGCCCAAGCCCCGCCTCGGC
CCCCAGGGCAGCGGCCCGCAGAGCTCCACCCGGCAGGCGCCCGGGAAACT
CCGCCCCCCGGCCGGCAGGGGGCGCGCGCGCCGCCGGCCCCGCCCCGTGG
ACGCGGGTTCCGTGGGCGTTCCCGCGGCCAGGCATCAGCAATCTATCAGG
GAACGGCGGTGGCCGGTGCGGCGTGTTCGGTGGCGGCTCTGGCCGCTCAG
GCGCCTGCGGCTGGGTGAGCGCACGCGAGGCGGCGAGGCGGCAGCGTGTT
TCTAGGTCGTGGCGTCGGGCTTCCGGAGCTTTGGCGGCAGCTAGGGGAGG
ATG

TNFα

Tumor necrosis factor is a cytokine produced primarily by activated macrophages (Ml type) and other cells including CD4+ lymphocytes, NK cells and neurons (Pfeffer K. 2003 Cytokine Growth Factor Rev. 14(3-4):185-91) to regulate immune cells during an acute inflammatory response. TNF was originally characterized its ability to induce tumor cell apoptosis and cachexia, however, its roles are now recognized to impart both beneficial (inflammation and in protective immune responses against a variety of infectious pathogens) and detrimental effects (sepsis, cancer, autoimmune disease). TNF, an endogenous pyrogen, induces fever, apoptotic cell death, cachexia, inflammation, inhibits tumorigenesis and viral replication and mediates sepsis by responding to IL-1 and IL-6 producing cells. Dysregulation of TNF production has been implicated in a variety of human diseases including Alzheimer's disease, cancer, major depression and inflammatory bowel disease (IBD). TNFα can be produced ectopically in the setting of malignancy and parallels parathyroid hormone both in causing secondary hypercalcemia and in the cancers with which excessive production is associated.

Protein: TNFα Gene: TNFα (Homo sapiens, chromosome 6, 31543344-31546113 [NCBI Reference Sequence: NC_{—}000006.11]; start site location: 31543519; strand: positive)

Gene Identification

| | |
|---|---|
| GeneID | 7124 |
| HGNC | 11892 |
| HPRD | 01855 |
| MIM | 191160 |

Targeted Sequences

| | | | Relative |
|---|---|---|---|
| | | | upstream |
| | | | location |
| | De- | | to gene |
| Sequence | sign | | start |
| ID No: | ID | Sequence (5′-3′) | site |
| 10095 | | CGGGGAAAGAATCATTCAACCAGCGG | 229 |
| 10096 | TNF1 | CGGTTTCTTCTCCATCGCGGGGGCG | 326 |
| 10129 | | CTGCTCCGATTCCGAGGGGGGTCTTCT | 412 |
| 10154 | | CTCCGTGTGGGGCTCTGGTCGGCAGCT | 1464 |
| 10207 | | CGCAGCCCCGTGGTACATCGAGTGCAGC | 2151 |

Target Shift Sequences

| | | Relative |
|---|---|---|
| | | upstream |
| | | location to |
| Sequence | | gene start |
| ID No: | Sequence (5′-3′) | site |
| 10095 | CGGGGAAAGAATCATTCAACCAGCGG | 229 |
| 10096 | CGGTTTCTTCTCCATCGCGGGGGCG | 326 |
| 10097 | GGTTTCTTCTCCATCGCGGG | 327 |
| 10098 | GTTTCTTCTCCATCGCGGGG | 328 |
| 10099 | TTTCTTCTCCATCGCGGGGG | 329 |
| 10100 | TTCTTCTCCATCGCGGGGGC | 330 |
| 10101 | TCTTCTCCATCGCGGGGGCG | 331 |
| 10102 | CTTCTCCATCGCGGGGGCGG | 332 |
| 10103 | TTCTCCATCGCGGGGGCGGG | 333 |
| 10104 | TCTCCATCGCGGGGGCGGGG | 334 |
| 10105 | CTCCATCGCGGGGGCGGGGA | 335 |
| 10106 | TCCATCGCGGGGGCGGGGAT | 336 |
| 10107 | CCATCGCGGGGGCGGGGATT | 337 |
| 10108 | CATCGCGGGGGCGGGGATTT | 338 |
| 10109 | ATCGCGGGGGCGGGGATTTG | 339 |
| 10110 | TCGCGGGGGCGGGGATTTGG | 340 |
| 10111 | TCGGTTTCTTCTCCATCGCG | 325 |
| 10112 | CTCGGTTTCTTCTCCATCGC | 324 |
| 10113 | TCTCGGTTTCTTCTCCATCG | 323 |
| 10114 | GTCTCGGTTTCTTCTCCATC | 322 |
| 10115 | TGTCTCGGTTTCTTCTCCAT | 321 |
| 10116 | CTGTCTCGGTTTCTTCTCCA | 320 |
| 10117 | TCTGTCTCGGTTTCTTCTCC | 319 |
| 10118 | TTCTGTCTCGGTTTCTTCTC | 318 |
| 10119 | CTTCTGTCTCGGTTTCTTCT | 317 |
| 10120 | CCTTCTGTCTCGGTTTCTTC | 316 |
| 10121 | ACCTTCTGTCTCGGTTTCTT | 315 |
| 10122 | CACCTTCTGTCTCGGTTTCT | 314 |
| 10123 | GCACCTTCTGTCTCGGTTTC | 313 |
| 10124 | TGCACCTTCTGTCTCGGTTT | 312 |
| 10125 | CTGCACCTTCTGTCTCGGTT | 311 |
| 10126 | CCTGCACCTTCTGTCTCGGT | 310 |
| 10127 | CCCTGCACCTTCTGTCTCGG | 309 |
| 10128 | GCCCTGCACCTTCTGTCTCG | 308 |
| 10129 | CTGCTCCGATTCCGAGGGGGGTCTTCT | 412 |
| 10130 | TGCTCCGATTCCGAGGGGGG | 413 |
| 10131 | GCTCCGATTCCGAGGGGGGT | 414 |
| 10132 | CTCCGATTCCGAGGGGGGTC | 415 |
| 10133 | TCCGATTCCGAGGGGGGTCT | 416 |
| 10134 | CCGATTCCGAGGGGGGTCTT | 417 |
| 10135 | CGATTCCGAGGGGGGTCTTC | 418 |
| 10136 | GATTCCGAGGGGGGTCTTCT | 419 |
| 10137 | ATTCCGAGGGGGGTCTTCTG | 420 |
| 10138 | TTCCGAGGGGGGTCTTCTGG | 421 |
| 10139 | TCCGAGGGGGGTCTTCTGGG | 422 |
| 10140 | CCGAGGGGGGTCTTCTGGGC | 423 |
| 10141 | CGAGGGGGGTCTTCTGGGCC | 424 |
| 10142 | CCTGCTCCGATTCCGAGGGG | 411 |
| 10143 | CCCTGCTCCGATTCCGAGGG | 410 |
| 10144 | TCCCTGCTCCGATTCCGAGG | 409 |
| 10145 | CTCCCTGCTCCGATTCCGAG | 408 |
| 10146 | CCTCCCTGCTCCGATTCCGA | 407 |
| 10147 | TCCTCCCTGCTCCGATTCCG | 406 |
| 10148 | ATCCTCCCTGCTCCGATTCC | 405 |
| 10149 | CATCCTCCCTGCTCCGATTC | 404 |
| 10150 | CCATCCTCCCTGCTCCGATT | 403 |
| 10151 | CCCATCCTCCCTGCTCCGAT | 402 |
| 10152 | CCCCATCCTCCCTGCTCCGA | 401 |
| 10153 | TCCCCATCCTCCCTGCTCCG | 400 |
| 10154 | CTCCGTGTGGGGCTCTGGTCGGCAGCT | 1464 |
| 10155 | TCCGTGTGGGGCTCTGGTCG | 1465 |
| 10156 | CCGTGTGGGGCTCTGGTCGG | 1466 |
| 10157 | CGTGTGGGGCTCTGGTCGGC | 1467 |
| 10158 | GTGTGGGGCTCTGGTCGGCA | 1468 |
| 10159 | TGTGGGGCTCTGGTCGGCAG | 1469 |
| 10160 | GTGGGGCTCTGGTCGGCAGC | 1470 |
| 10161 | TGGGGCTCTGGTCGGCAGCT | 1471 |
| 10162 | GGGGCTCTGGTCGGCAGCTG | 1472 |
| 10163 | GGGCTCTGGTCGGCAGCTGG | 1473 |
| 10164 | GGCTCTGGTCGGCAGCTGGC | 1474 |
| 10165 | GCTCTGGTCGGCAGCTGGCT | 1475 |
| 10166 | CTCTGGTCGGCAGCTGGCTT | 1476 |
| 10167 | TCTGGTCGGCAGCTGGCTTT | 1477 |
| 10168 | CTGGTCGGCAGCTGGCTTTC | 1478 |
| 10169 | TGGTCGGCAGCTGGCTTTCA | 1479 |
| 10170 | GGTCGGCAGCTGGCTTTCAG | 1480 |
| 10171 | GTCGGCAGCTGGCTTTCAGA | 1481 |
| 10172 | TCGGCAGCTGGCTTTCAGAG | 1482 |
| 10173 | CGGCAGCTGGCTTTCAGAGC | 1483 |
| 10174 | CCTCCGTGTGGGGCTCTGGT | 1463 |
| 10175 | GCCTCCGTGTGGGGCTCTGG | 1462 |
| 10176 | TGCCTCCGTGTGGGGCTCTG | 1461 |
| 10177 | ATGCCTCCGTGTGGGGCTCT | 1460 |
| 10178 | GATGCCTCCGTGTGGGGCTC | 1459 |
| 10179 | AGATGCCTCCGTGTGGGGCT | 1458 |
| 10180 | CAGATGCCTCCGTGTGGGGC | 1457 |
| 10181 | GCAGATGCCTCCGTGTGGGG | 1456 |
| 10182 | TGCAGATGCCTCCGTGTGGG | 1455 |
| 10183 | GTGCAGATGCCTCCGTGTGG | 1454 |
| 10184 | GGTGCAGATGCCTCCGTGTG | 1453 |
| 10185 | GGGTGCAGATGCCTCCGTGT | 1452 |
| 10186 | AGGGTGCAGATGCCTCCGTG | 1451 |
| 10187 | GAGGGTGCAGATGCCTCCGT | 1450 |
| 10188 | CGAGGGTGCAGATGCCTCCG | 1449 |
| 10189 | TCGAGGGTGCAGATGCCTCC | 1448 |
| 10190 | ATCGAGGGTGCAGATGCCTC | 1447 |
| 10191 | CATCGAGGGTGCAGATGCCT | 1446 |
| 10192 | TCATCGAGGGTGCAGATGCC | 1445 |
| 10193 | TTCATCGAGGGTGCAGATGC | 1444 |
| 10194 | CTTCATCGAGGGTGCAGATG | 1443 |
| 10195 | GCTTCATCGAGGGTGCAGAT | 1442 |
| 10196 | GGCTTCATCGAGGGTGCAGA | 1441 |
| 10197 | GGGCTTCATCGAGGGTGCAG | 1440 |
| 10198 | TGGGCTTCATCGAGGGTGCA | 1439 |
| 10199 | TTGGGCTTCATCGAGGGTGC | 1438 |
| 10200 | ATTGGGCTTCATCGAGGGTG | 1437 |
| 10201 | TATTGGGCTTCATCGAGGGT | 1436 |
| 10202 | TTATTGGGCTTCATCGAGGG | 1435 |
| 10203 | TTTATTGGGCTTCATCGAGG | 1434 |
| 10204 | GTTTATTGGGCTTCATCGAG | 1433 |
| 10205 | GGTTTATTGGGCTTCATCGA | 1432 |
| 10206 | AGGTTTATTGGGCTTCATCG | 1431 |
| 10207 | CGCAGCCCCGTGGTACATCGAGTGCAGC | 2151 |
| 10208 | GCAGCCCCGTGGTACATCGA | 2152 |
| 10209 | CAGCCCCGTGGTACATCGAG | 2153 |
| 10210 | AGCCCCGTGGTACATCGAGT | 2154 |
| 10211 | GCCCCGTGGTACATCGAGTG | 2155 |
| 10212 | CCCCGTGGTACATCGAGTGC | 2156 |
| 10213 | CCCGTGGTACATCGAGTGCA | 2157 |
| 10214 | CCGTGGTACATCGAGTGCAG | 2158 |
| 10215 | CGTGGTACATCGAGTGCAGC | 2159 |
| 10216 | GTGGTACATCGAGTGCAGCC | 2160 |
| 10217 | TGGTACATCGAGTGCAGCCA | 2161 |
| 10218 | GGTACATCGAGTGCAGCCAG | 2162 |
| 10219 | GTACATCGAGTGCAGCCAGG | 2163 |
| 10220 | TACATCGAGTGCAGCCAGGG | 2164 |
| 10221 | ACATCGAGTGCAGCCAGGGT | 2165 |
| 10222 | CATCGAGTGCAGCCAGGGTT | 2166 |
| 10223 | ATCGAGTGCAGCCAGGGTTC | 2167 |
| 10224 | TCGAGTGCAGCCAGGGTTCC | 2168 |
| 10225 | CGAGTGCAGCCAGGGTTCCT | 2169 |
| 10226 | ACGCAGCCCCGTGGTACATC | 2150 |
| 10227 | AACGCAGCCCCGTGGTACAT | 2149 |
| 10228 | GAACGCAGCCCCGTGGTACA | 2148 |
| 10229 | GGAACGCAGCCCCGTGGTAC | 2147 |
| 10230 | TGGAACGCAGCCCCGTGGTA | 2146 |
| 10231 | CTGGAACGCAGCCCCGTGGT | 2145 |
| 10232 | GCTGGAACGCAGCCCCGTGG | 2144 |
| 10233 | AGCTGGAACGCAGCCCCGTG | 2143 |
| 10234 | GAGCTGGAACGCAGCCCCGT | 2142 |
| 10235 | TGAGCTGGAACGCAGCCCCG | 2141 |
| 10236 | GTGAGCTGGAACGCAGCCCC | 2140 |
| 10237 | GGTGAGCTGGAACGCAGCCC | 2139 |
| 10238 | GGGTGAGCTGGAACGCAGCC | 2138 |
| 10239 | TGGGTGAGCTGGAACGCAGC | 2137 |
| 10240 | CTGGGTGAGCTGGAACGCAG | 2136 |
| 10241 | CCTGGGTGAGCTGGAACGCA | 2135 |
| 10242 | CCCTGGGTGAGCTGGAACGC | 2134 |
| 10243 | TCCCTGGGTGAGCTGGAACG | 2133 |

Hot Zones (Relative upstream location to gene start site)
168-450
1430-1520
2150-2240

### Examples

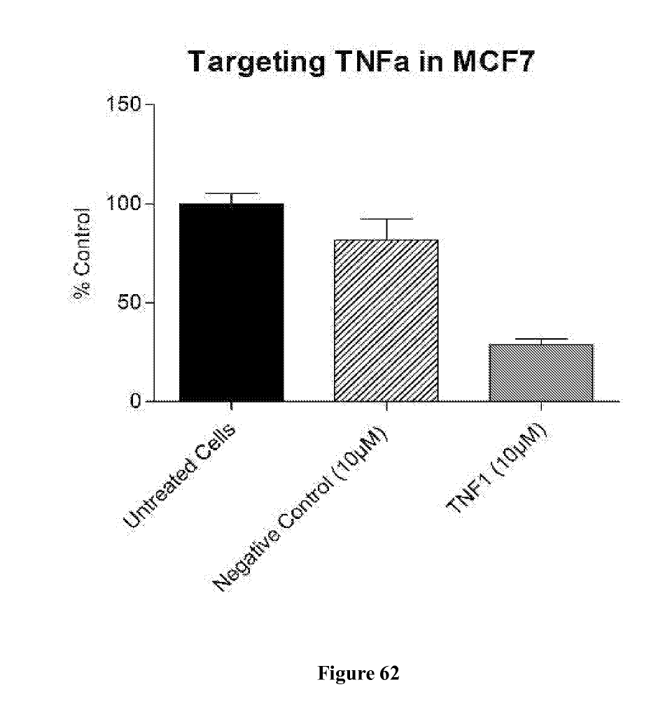

In FIG. 62, In MCF7 (human mammary breast cell line), TNF1 (312) produced statistically significant (P<0.05) inhibition at 10 μM compared to the untreated and negative control values. The TNFα sequence TNF1 (312) fits the independent and dependent DNAi motif claims.

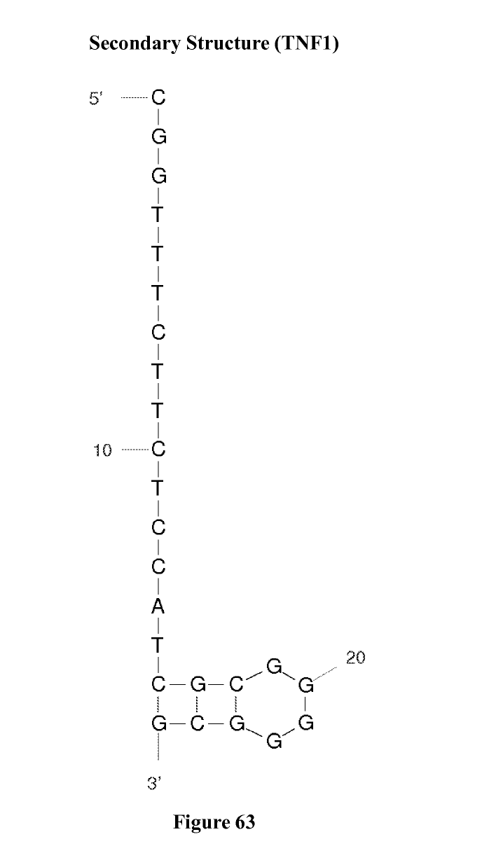

The secondary structure for TNF1 (312) is shown in FIG. 63.

Genetic Code (5′ Upstream Region)

(SEQ ID NO: 11982)

CTCACTGTCTCTCTCTCTCTCTCTCTTTCTCTGCAGGTTCTCCCCATGAC
ACCACCTGAACGTCTCTTCCTCCCAAGGGTGTGTGGCACCACCCTACACC
TCCTCCTTCTGGGGCTGCTGCTGGTTCTGCTGCCTGGGGCCCAGGTGAGG
CAGCAGGAGAATGGGGGCTGCTGGGGTGGCTCAGCCAAACCTTGAGCCCT
AGAGCCCCCCTCAACTCTGTTCTCCCCTAGGGGCTCCCTGGTGTTGGCCT
CACACCTTCAGCTGCCCAGACTGCCCGTCAGCACCCCAAGATGCATCTTG
CCCACAGCACCCTCAAACCTGCTGCTCACCTCATTGGTAAACATCCACCT
GACCTCCCAGACATGTCCCCACCAGCTCTCCTCCTACCCCTGCCTCAGGA
ACCCAAGCATCCACCCCTCTCCCCCAACTTCCCCCACGCTAAAAAAAACA
GAGGGAGCCCACTCCTATGCCTCCCCCTGCCATCCCCCAGGAACTCAGTT
GTTCAGTGCCCACTTCCTCAGGGATTGAGACCTCTGATCCAGACCCCTGA
TCTCCCACCCCCATCCCCTATGGCTCTTCCTAGGAGACCCCAGCAAGCAG
AACTCACTGCTCTGGAGAGCAAACACGGACCGTGCCTTCCTCCAGGATGG
TTTCTCCTTGAGCAACAATTCTCTCCTGGTCCCCACCAGTGGCATCTACT
TCGTCTACTCCCAGGTGGTCTTCTCTGGGAAAGCCTACTCTCCCAAGGCC
ACCTCCTCCCCACTCTACCTGGCCCATGAGGTCCAGCTCTTCTCCTCCCA
GTACCCCTTCCATGTGCCTCTCCTCAGCTCCCAGAAGATGGTGTATCCAG
GGCTGCAGGAACCCTGGCTGCACTCGATGTACCACGGGGCTGCGTTCCAG
CTCACCCAGGGAGACCAGCTATCCACCCACACAGATGGCATCCCCCACCT
AGTCCTCAGCCCTAGTACTGTCTTCTTTGGAGCCTTCGCTCTGTAGAACT
TGGAAAAATCCAGAAAGAAAAAATAATTGATTTCAAGACCTTCTCCCCAT
TCTGCCTCCATTCTGACCATTTCAGGGGTCGTCACCACCTCTCCTTTGGC
CATTCCAACAGCTCAAGTCTTCCCTGATCAAGTCACCGGAGCTTTCAAAG
AAGGAATTCTAGGCATCCCAGGGGACCACACCTCCCTGAACCATCCCTGA
TGTCTGTCTGGCTGAGGATTTCAAGCCTGCCTAGGAATTCCCAGCCCAAA
GCTGTTGGTCTGTCCCACCAGCTAGGTGGGGCCTAGATCCACACACAGAG
GAAGAGCAGGCACATGGAGGAGCTTGGGGGATGACTAGAGGCAGGGAGGG
GACTATTTATGAAGGCAAAAAAATTAAATTATTTATTTATGGAGGATGGA
GAGAGGGGAATAATAGAAGAACATCCAAGGAGAAACAGAGACAGGCCCAA
GAGATGAAGAGTGAGAGGGCATGCGCACAAGGCTGACCAAGAGAGAAAGA
AGTAGGCATGAGGGATCACAGGGCCCCAGAAGGCAGGGAAAGGCTCTGAA
AGCCAGCTGCCGACCAGAGCCCCACACGGAGGCATCTGCACCCTCGATGA
AGCCCAATAAACCTCTTTTCTCTGAAATGCTGTCTGCTTGTGTGTGTGTG
TCTGGGAGTGAGAACTTCCCAGTCTATCTAAGGAATGGAGGGAGGGACAG
AGGGCTCAAAGGGAGCAAGAGCTGTGGGGAGAACAAAAGGATAAGGGCTC
AGAGAGCTTCAGGGATATGTGATGGACTCACCAGGTGAGGCCGCCAGACT
GCTGCAGGGGAAGCAAAGGAGAAGCTGAGAAGATGAAGGAAAAGTCAGGG
TCTGGAGGGGCGGGGGTCAGGGAGCTCCTGGGAGATATGGCCACATGTAG
CGGCTCTGAGGAATGGGTTACAGGAGACCTCTGGGGAGATGTGACCACAG
CAATGGGTAGGAGAATGTCCAGGGCTATGGAAGTCGAGTATGGGGACCCC
CCCTTAACGAAGACAGGGCCATGTAGAGGGCCCCAGGGAGTGAAAGAGCC
TCCAGGACCTCCAGGTATGGAATACAGGGGACGTTTAAGAAGATATGGCC
ACACACTGGGGCCCTGAGAAGTGAGAGCTTCATGAAAAAAATCAGGGACC
CCAGAGTTCCTTGGAAGCCAAGACTGAAACCAGCATTATGAGTCTCCGGG
TCAGAATGAAAGAAGAAGGCCTGCCCCAGTGGGGTCTGTGAATTCCCGGG
GGTGATTTCACTCCCCGGGGCTGTCCCAGGCTTGTCCCTGCTACCCCCAC
CCAGCCTTTCCTGAGGCCTCAAGCCTGCCACCAAGCCCCCAGCTCCTTCT
CCCCGCAGGGACCCAAACACAGGCCTCAGGACTCAACACAGCTTTTCCCT
CCAACCCCGTTTTCTCTCCCTCAAGGACTCAGCTTTCTGAAGCCCCTCCC
AGTTCTAGTTCTATCTTTTTCCTGCATCCTGTCTGGAAGTTAGAAGGAAA
CAGACCACAGACCTGGTCCCCAAAAGAAATGGAGGCAATAGGTTTTGAGG
GGCATGGGGACGGGGTTCAGCCTCCAGGGTCCTACACACAAATCAGTCAG
TGGCCCAGAAGACCCCCCTCGGAATCGGAGCAGGGAGGATGGGGAGTGTG
AGGGGTATCCTTGATGCTTGTGTGTCCCCAACTTTCCAAATCCCCGCCCC
CGCGATGGAGAAGAAACCGAGACAGAAGGTGCAGGGCCCACTACCGCTTC
CTCCAGATGAGCTCATGGGTTTCTCCACCAAGGAAGTTTTCCGCTGGTTG
AATGATTCTTTCCCCGCCCTCCTCTCGCCCCAGGGACATATAAAGGCAGT
TGTTGGCACACCCAGCCAGCAGACGCTCCCTCAGCAAGGACAGCAGAGGA
CCAGCTAAGAGGGAGAGAAGCAACTACAGACCCCCCCTGAAAACAACCCT
CAGACGCCACATCCCCTGACAAGCTGCCAGGCAGGTTCTCTTCCTCTCAC
ATACTGACCCACGGCTCCACCCTCTCTCCCCTGGAAAGGACACCATG

ITGA4

Integrins are ubiquitously expressed adhesion molecules. They are cell-surface receptors that exist as heterodimers of alpha and beta subunits. ITGA4 encodes an alpha 4 chain. Unlike other integrin alpha chains, alpha 4 neither contains an I-domain, nor undergoes disulfide-linked cleavage. Alpha 4 chain associates with either beta 1 chain or beta 7 chain. At physiological conditions, integrins are highly glycosylated and contain a Ca2+ or Mg2+ ion, which is essential for ligand binding. Integrin receptors are critical for cell attachment to the extracellular matrix (ECM) and this is mediated through integrin-fibronectin, -vitronectin, -collagen and -laminin interactions. Intracellularly, integrins form adhesion complexes with proteins including talin, vinculin, paxillin and alpha-actinin. They also regulate kinases, such as focal adhesion kinase and Src family kinases, to mediate attachment to the actin cytoskeleton. Integrins also have a significant role in cell signaling and can activate protein kinases involved in the regulation of cell growth, division, survival, differentiation, migration and apoptosis. Glycoprotein II/IIIb (alphaIIbeta3) is an integrin receptor found on the surface of platelets. It is involved in the cross-linking of platelets with fibrin, and so has a vital role in blood clot formation.

Protein: ITGA4 Gene: ITGA4 (CD49D) (Homo sapiens, chromosome 2, 182321619-182402474 [NCBI Reference Sequence: NC_{—}000002.11]; start site location: 182322383; strand: positive)

Gene Identification

| | |
|---|---|
| GeneID | 3676 |
| HGNC | 6140 |
| MIM | 192975 |

Targeted Sequences

| | | | Relative |
|---|---|---|---|
| | | | upstream |
| | | | location |
| Se- | | | to gene |
| quence | Design | | start |
| ID No: | ID | Sequence (5′-3′) | site |
| 10244 | | GCGCTCTCGGTGGGGAACATTCAACAC | 1 |
| 10252 | | CGGGATGCGACGGTTGGCCAACGG | 54 |
| 10278 | | CGCAGCGTGTCCGGCGCCAGCGGGC | 102 |
| 10299 | | CGGCCCACCGCGGGCGGAGCGTTCG | 160 |
| 10449 | | CGCGCACTCGCCCGGCCCCACTCCCG | 201 |
| 10599 | | CGCCAGCCGGGAGCTTCGGGTGCTCGCG | 235 |
| 10749 | | CGGGTACGGGCCGCTGGGTGGGGTCCCG | 272 |
| 10899 | | GTGCGGAGGCGCAGGGCCGGGCTCCG | 306 |
| 10900 | | CTACGCGCGGCTGCAGGGGGCGC | 339 |
| 10938 | | CTGCGCAGGACTCGCGTCCTGGCCCG | 375 |
| 11009 | | CCCGCAGAGCGCGGGATGGCTC | 411 |
| 11080 | | CGGACCTGATGGGGCACGGGCTTCCCC | 448 |
| 11117 | | CGGTGGTTGGGGCCTAGAAGCG | 481 |
| 11154 | | CGCGCCCCTCGCTGTGACCGCCCAGCCCG | 524 |
| 11203 | | CGGGGAGTGGGACTGCGGCGGGGAGCCG | 580 |
| 11208 | | ACTCGCCGAAGGCCCCTGGGGAAC | 718 |
| 11222 | | CGGGCTGCATGCGTGAGCAGG | 840 |
| 11252 | | CGGCAGGCGGTTTAGGCTGTGGCTG | 885 |
| 11278 | | CCGATTCGGATTGCTCCAGCTGG | 962 |
| 11289 | | CGCACCCACTCAGTTGCCACGGG | 1008 |
| 11327 | | CGGAGACCCACAACGCAACACACC | 1099 |

Target Shift Sequences

| | | Relative |
|---|---|---|
| | | upstream |
| | | location to |
| Sequence | | gene start |
| ID No: | Sequence (5′-3′) | site |
| 10244 | GCGCTCTCGGTGGGGAACATTCAACAC | 1 |
| 10245 | CGCTCTCGGTGGGGAACATT | 2 |
| 10246 | GCTCTCGGTGGGGAACATTC | 3 |
| 10247 | CTCTCGGTGGGGAACATTCA | 4 |
| 10248 | TCTCGGTGGGGAACATTCAA | 5 |
| 10249 | CTCGGTGGGGAACATTCAAC | 6 |
| 10250 | TCGGTGGGGAACATTCAACA | 7 |
| 10251 | CGGTGGGGAACATTCAACAC | 8 |
| 10252 | CGGGATGCGACGGTTGGCCAACGG | 54 |
| 10253 | GGGATGCGACGGTTGGCCAA | 55 |
| 10254 | GGATGCGACGGTTGGCCAAC | 56 |
| 10255 | GATGCGACGGTTGGCCAACG | 57 |
| 10256 | ATGCGACGGTTGGCCAACGG | 58 |
| 10257 | TGCGACGGTTGGCCAACGGG | 59 |
| 10258 | GCGACGGTTGGCCAACGGGG | 60 |
| 10259 | CGACGGTTGGCCAACGGGGA | 61 |
| 10260 | ACGGGATGCGACGGTTGGCC | 53 |
| 10261 | CACGGGATGCGACGGTTGGC | 52 |
| 10262 | GCACGGGATGCGACGGTTGG | 51 |
| 10263 | TGCACGGGATGCGACGGTTG | 50 |
| 10264 | TTGCACGGGATGCGACGGTT | 49 |
| 10265 | GTTGCACGGGATGCGACGGT | 48 |
| 10266 | AGTTGCACGGGATGCGACGG | 47 |
| 10267 | AAGTTGCACGGGATGCGACG | 46 |
| 10268 | AAAGTTGCACGGGATGCGAC | 45 |
| 10269 | CAAAGTTGCACGGGATGCGA | 44 |
| 10270 | CCAAAGTTGCACGGGATGCG | 43 |
| 10271 | CCCAAAGTTGCACGGGATGC | 42 |
| 10272 | CCCCAAAGTTGCACGGGATG | 41 |
| 10273 | ACCCCAAAGTTGCACGGGAT | 40 |
| 10274 | TACCCCAAAGTTGCACGGGA | 39 |
| 10275 | CTACCCCAAAGTTGCACGGG | 38 |
| 10276 | ACTACCCCAAAGTTGCACGG | 37 |
| 10277 | CACTACCCCAAAGTTGCACG | 36 |
| 10278 | CGCAGCGTGTCCGGCGCCAGCGGGC | 102 |
| 10279 | GCAGCGTGTCCGGCGCCAGC | 103 |
| 10280 | CAGCGTGTCCGGCGCCAGCG | 104 |
| 10281 | AGCGTGTCCGGCGCCAGCGG | 105 |
| 10282 | GCGTGTCCGGCGCCAGCGGG | 106 |
| 10283 | CGTGTCCGGCGCCAGCGGGC | 107 |
| 10284 | GTGTCCGGCGCCAGCGGGCT | 108 |
| 10285 | TGTCCGGCGCCAGCGGGCTA | 109 |
| 10286 | GTCCGGCGCCAGCGGGCTAA | 110 |
| 10287 | TCCGGCGCCAGCGGGCTAAA | 111 |
| 10288 | CCGGCGCCAGCGGGCTAAAG | 112 |
| 10289 | CGGCGCCAGCGGGCTAAAGG | 113 |
| 10290 | GCGCAGCGTGTCCGGCGCCA | 101 |
| 10291 | GGCGCAGCGTGTCCGGCGCC | 100 |
| 10292 | AGGCGCAGCGTGTCCGGCGC | 99 |
| 10293 | GAGGCGCAGCGTGTCCGGCG | 98 |
| 10294 | TGAGGCGCAGCGTGTCCGGC | 97 |
| 10295 | ATGAGGCGCAGCGTGTCCGG | 96 |
| 10296 | GATGAGGCGCAGCGTGTCCG | 95 |
| 10297 | AGATGAGGCGCAGCGTGTCC | 94 |
| 10298 | GAGATGAGGCGCAGCGTGTC | 93 |
| 10299 | CGGCCCACCGCGGGCGGAGCGTTCG | 160 |
| 10300 | GGCCCACCGCGGGCGGAGCG | 161 |
| 10301 | GCCCACCGCGGGCGGAGCGT | 162 |
| 10302 | CCCACCGCGGGCGGAGCGTT | 163 |
| 10303 | CCACCGCGGGCGGAGCGTTC | 164 |
| 10304 | CACCGCGGGCGGAGCGTTCG | 165 |
| 10305 | ACCGCGGGCGGAGCGTTCGG | 166 |
| 10306 | CCGCGGGCGGAGCGTTCGGG | 167 |
| 10307 | CGCGGGCGGAGCGTTCGGGC | 168 |
| 10308 | GCGGGCGGAGCGTTCGGGCC | 169 |
| 10309 | CGGGCGGAGCGTTCGGGCCG | 170 |
| 10310 | GGGCGGAGCGTTCGGGCCGG | 171 |
| 10311 | GGCGGAGCGTTCGGGCCGGC | 172 |
| 10312 | GCGGAGCGTTCGGGCCGGCC | 173 |
| 10313 | CGGAGCGTTCGGGCCGGCCT | 174 |
| 10314 | GGAGCGTTCGGGCCGGCCTG | 175 |
| 10315 | GAGCGTTCGGGCCGGCCTGG | 176 |
| 10316 | AGCGTTCGGGCCGGCCTGGG | 177 |
| 10317 | GCGTTCGGGCCGGCCTGGGA | 178 |
| 10318 | CGTTCGGGCCGGCCTGGGAT | 179 |
| 10319 | GTTCGGGCCGGCCTGGGATG | 180 |
| 10320 | TTCGGGCCGGCCTGGGATGC | 181 |
| 10321 | TCGGGCCGGCCTGGGATGCC | 182 |
| 10322 | CGGGCCGGCCTGGGATGCCG | 183 |
| 10323 | GGGCCGGCCTGGGATGCCGC | 184 |
| 10324 | GGCCGGCCTGGGATGCCGCG | 185 |
| 10325 | GCCGGCCTGGGATGCCGCGC | 186 |
| 10326 | CCGGCCTGGGATGCCGCGCA | 187 |
| 10327 | CGGCCTGGGATGCCGCGCAC | 188 |
| 10328 | GGCCTGGGATGCCGCGCACT | 189 |
| 10329 | GCCTGGGATGCCGCGCACTC | 190 |
| 10330 | CCTGGGATGCCGCGCACTCG | 191 |
| 10331 | CTGGGATGCCGCGCACTCGC | 192 |
| 10332 | TGGGATGCCGCGCACTCGCC | 193 |
| 10333 | GGGATGCCGCGCACTCGCCC | 194 |
| 10334 | GGATGCCGCGCACTCGCCCG | 195 |
| 10335 | GATGCCGCGCACTCGCCCGG | 196 |
| 10336 | ATGCCGCGCACTCGCCCGGC | 197 |
| 10337 | TGCCGCGCACTCGCCCGGCC | 198 |
| 10338 | GCCGCGCACTCGCCCGGCCC | 199 |
| 10339 | CCGCGCACTCGCCCGGCCCC | 200 |
| 10340 | CGCGCACTCGCCCGGCCCCA | 201 |
| 10341 | GCGCACTCGCCCGGCCCCAC | 202 |
| 10342 | CGCACTCGCCCGGCCCCACT | 203 |
| 10343 | GCACTCGCCCGGCCCCACTC | 204 |
| 10344 | CACTCGCCCGGCCCCACTCC | 205 |
| 10345 | ACTCGCCCGGCCCCACTCCC | 206 |
| 10346 | CTCGCCCGGCCCCACTCCCG | 207 |
| 10347 | TCGCCCGGCCCCACTCCCGG | 208 |
| 10348 | CGCCCGGCCCCACTCCCGGT | 209 |
| 10349 | GCCCGGCCCCACTCCCGGTT | 210 |
| 10350 | CCCGGCCCCACTCCCGGTTT | 211 |
| 10351 | CCGGCCCCACTCCCGGTTTC | 212 |
| 10352 | CGGCCCCACTCCCGGTTTCT | 213 |
| 10353 | GGCCCCACTCCCGGTTTCTG | 214 |
| 10354 | GCCCCACTCCCGGTTTCTGC | 215 |
| 10355 | CCCCACTCCCGGTTTCTGCC | 216 |
| 10356 | CCCACTCCCGGTTTCTGCCG | 217 |
| 10357 | CCACTCCCGGTTTCTGCCGC | 218 |
| 10358 | CACTCCCGGTTTCTGCCGCC | 219 |
| 10359 | ACTCCCGGTTTCTGCCGCCA | 220 |
| 10360 | CTCCCGGTTTCTGCCGCCAG | 221 |
| 10361 | TCCCGGTTTCTGCCGCCAGC | 222 |
| 10362 | CCCGGTTTCTGCCGCCAGCC | 223 |
| 10363 | CCGGTTTCTGCCGCCAGCCG | 224 |
| 10364 | CGGTTTCTGCCGCCAGCCGG | 225 |
| 10365 | GGTTTCTGCCGCCAGCCGGG | 226 |
| 10366 | GTTTCTGCCGCCAGCCGGGA | 227 |
| 10367 | TTTCTGCCGCCAGCCGGGAG | 228 |
| 10368 | TTCTGCCGCCAGCCGGGAGC | 229 |
| 10369 | TCTGCCGCCAGCCGGGAGCT | 230 |
| 10370 | CTGCCGCCAGCCGGGAGCTT | 231 |
| 10371 | TGCCGCCAGCCGGGAGCTTC | 232 |
| 10372 | GCCGCCAGCCGGGAGCTTCG | 233 |
| 10373 | CCGCCAGCCGGGAGCTTCGG | 234 |
| 10374 | CGCCAGCCGGGAGCTTCGGG | 235 |
| 10375 | GCCAGCCGGGAGCTTCGGGT | 236 |
| 10376 | CCAGCCGGGAGCTTCGGGTG | 237 |
| 10377 | CAGCCGGGAGCTTCGGGTGC | 238 |
| 10378 | AGCCGGGAGCTTCGGGTGCT | 239 |
| 10379 | GCCGGGAGCTTCGGGTGCTC | 240 |
| 10380 | CCGGGAGCTTCGGGTGCTCG | 241 |
| 10381 | CGGGAGCTTCGGGTGCTCGC | 242 |
| 10382 | GGGAGCTTCGGGTGCTCGCG | 243 |
| 10383 | GGAGCTTCGGGTGCTCGCGC | 244 |
| 10384 | GAGCTTCGGGTGCTCGCGCT | 245 |
| 10385 | AGCTTCGGGTGCTCGCGCTG | 246 |
| 10386 | GCTTCGGGTGCTCGCGCTGC | 247 |
| 10387 | CTTCGGGTGCTCGCGCTGCT | 248 |
| 10388 | TTCGGGTGCTCGCGCTGCTT | 249 |
| 10389 | TCGGGTGCTCGCGCTGCTTC | 250 |
| 10390 | CGGGTGCTCGCGCTGCTTCT | 251 |
| 10391 | GGGTGCTCGCGCTGCTTCTC | 252 |
| 10392 | GGTGCTCGCGCTGCTTCTCC | 253 |
| 10393 | GTGCTCGCGCTGCTTCTCCG | 254 |
| 10394 | TGCTCGCGCTGCTTCTCCGG | 255 |
| 10395 | GCTCGCGCTGCTTCTCCGGG | 256 |
| 10396 | CTCGCGCTGCTTCTCCGGGT | 257 |
| 10397 | TCGCGCTGCTTCTCCGGGTA | 258 |
| 10398 | CGCGCTGCTTCTCCGGGTAC | 259 |
| 10399 | GCGCTGCTTCTCCGGGTACG | 260 |
| 10400 | CGCTGCTTCTCCGGGTACGG | 261 |
| 10401 | GCTGCTTCTCCGGGTACGGG | 262 |
| 10402 | CTGCTTCTCCGGGTACGGGC | 263 |
| 10403 | TGCTTCTCCGGGTACGGGCC | 264 |
| 10404 | GCTTCTCCGGGTACGGGCCG | 265 |
| 10405 | CTTCTCCGGGTACGGGCCGC | 266 |
| 10406 | TTCTCCGGGTACGGGCCGCT | 267 |
| 10407 | TCTCCGGGTACGGGCCGCTG | 268 |
| 10408 | CTCCGGGTACGGGCCGCTGG | 269 |
| 10409 | TCCGGGTACGGGCCGCTGGG | 270 |
| 10410 | CCGGGTACGGGCCGCTGGGT | 271 |
| 10411 | CGGGTACGGGCCGCTGGGTG | 272 |
| 10412 | GGGTACGGGCCGCTGGGTGG | 273 |
| 10413 | GGTACGGGCCGCTGGGTGGG | 274 |
| 10414 | GTACGGGCCGCTGGGTGGGG | 275 |
| 10415 | TACGGGCCGCTGGGTGGGGT | 276 |
| 10416 | ACGGGCCGCTGGGTGGGGTC | 277 |
| 10417 | CGGGCCGCTGGGTGGGGTCC | 278 |
| 10418 | GGGCCGCTGGGTGGGGTCCC | 279 |
| 10419 | GGCCGCTGGGTGGGGTCCCG | 280 |
| 10420 | GCCGCTGGGTGGGGTCCCGG | 281 |
| 10421 | CCGCTGGGTGGGGTCCCGGG | 282 |
| 10422 | CGCTGGGTGGGGTCCCGGGC | 283 |
| 10423 | GCTGGGTGGGGTCCCGGGCG | 284 |
| 10424 | CTGGGTGGGGTCCCGGGCGT | 285 |
| 10425 | TGGGTGGGGTCCCGGGCGTG | 286 |
| 10426 | GGGTGGGGTCCCGGGCGTGG | 287 |
| 10427 | GGTGGGGTCCCGGGCGTGGT | 288 |
| 10428 | GTGGGGTCCCGGGCGTGGTG | 289 |
| 10429 | TGGGGTCCCGGGCGTGGTGC | 290 |
| 10430 | GGGGTCCCGGGCGTGGTGCG | 291 |
| 10431 | GGGTCCCGGGCGTGGTGCGG | 292 |
| 10432 | GGTCCCGGGCGTGGTGCGGA | 293 |
| 10433 | GTCCCGGGCGTGGTGCGGAG | 294 |
| 10434 | TCCCGGGCGTGGTGCGGAGG | 295 |
| 10435 | CCCGGGCGTGGTGCGGAGGC | 296 |
| 10436 | CCGGGCGTGGTGCGGAGGCG | 297 |
| 10437 | CGGGCGTGGTGCGGAGGCGC | 298 |
| 10438 | TCGGCCCACCGCGGGCGGAG | 159 |
| 10439 | GTCGGCCCACCGCGGGCGGA | 158 |
| 10440 | AGTCGGCCCACCGCGGGCGG | 157 |
| 10441 | AAGTCGGCCCACCGCGGGCG | 156 |
| 10442 | GAAGTCGGCCCACCGCGGGC | 155 |
| 10443 | GGAAGTCGGCCCACCGCGGG | 154 |
| 10444 | GGGAAGTCGGCCCACCGCGG | 153 |
| 10445 | GGGGAAGTCGGCCCACCGCG | 152 |
| 10446 | AGGGGAAGTCGGCCCACCGC | 151 |
| 10447 | GAGGGGAAGTCGGCCCACCG | 150 |
| 10448 | GGAGGGGAAGTCGGCCCACC | 149 |
| 10449 | CGCGCACTCGCCCGGCCCCACTCCCG | 201 |
| 10450 | GCGCACTCGCCCGGCCCCAC | 202 |
| 10451 | CGCACTCGCCCGGCCCCACT | 203 |
| 10452 | GCACTCGCCCGGCCCCACTC | 204 |
| 10453 | CACTCGCCCGGCCCCACTCC | 205 |
| 10454 | ACTCGCCCGGCCCCACTCCC | 206 |
| 10455 | CTCGCCCGGCCCCACTCCCG | 207 |
| 10456 | TCGCCCGGCCCCACTCCCGG | 208 |
| 10457 | CGCCCGGCCCCACTCCCGGT | 209 |
| 10458 | GCCCGGCCCCACTCCCGGTT | 210 |
| 10459 | CCCGGCCCCACTCCCGGTTT | 211 |
| 10460 | CCGGCCCCACTCCCGGTTTC | 212 |
| 10461 | CGGCCCCACTCCCGGTTTCT | 213 |
| 10462 | GGCCCCACTCCCGGTTTCTG | 214 |
| 10463 | GCCCCACTCCCGGTTTCTGC | 215 |
| 10464 | CCCCACTCCCGGTTTCTGCC | 216 |
| 10465 | CCCACTCCCGGTTTCTGCCG | 217 |
| 10466 | CCACTCCCGGTTTCTGCCGC | 218 |
| 10467 | CACTCCCGGTTTCTGCCGCC | 219 |
| 10468 | ACTCCCGGTTTCTGCCGCCA | 220 |
| 10469 | CTCCCGGTTTCTGCCGCCAG | 221 |
| 10470 | TCCCGGTTTCTGCCGCCAGC | 222 |
| 10471 | CCCGGTTTCTGCCGCCAGCC | 223 |
| 10472 | CCGGTTTCTGCCGCCAGCCG | 224 |
| 10473 | CGGTTTCTGCCGCCAGCCGG | 225 |
| 10474 | GGTTTCTGCCGCCAGCCGGG | 226 |
| 10475 | GTTTCTGCCGCCAGCCGGGA | 227 |
| 10476 | TTTCTGCCGCCAGCCGGGAG | 228 |
| 10477 | TTCTGCCGCCAGCCGGGAGC | 229 |
| 10478 | TCTGCCGCCAGCCGGGAGCT | 230 |
| 10479 | CTGCCGCCAGCCGGGAGCTT | 231 |
| 10480 | TGCCGCCAGCCGGGAGCTTC | 232 |
| 10481 | GCCGCCAGCCGGGAGCTTCG | 233 |
| 10482 | CCGCCAGCCGGGAGCTTCGG | 234 |
| 10483 | CGCCAGCCGGGAGCTTCGGG | 235 |
| 10484 | GCCAGCCGGGAGCTTCGGGT | 236 |
| 10485 | CCAGCCGGGAGCTTCGGGTG | 237 |
| 10486 | CAGCCGGGAGCTTCGGGTGC | 238 |
| 10487 | AGCCGGGAGCTTCGGGTGCT | 239 |
| 10488 | GCCGGGAGCTTCGGGTGCTC | 240 |
| 10489 | CCGGGAGCTTCGGGTGCTCG | 241 |
| 10490 | CGGGAGCTTCGGGTGCTCGC | 242 |
| 10491 | GGGAGCTTCGGGTGCTCGCG | 243 |
| 10492 | GGAGCTTCGGGTGCTCGCGC | 244 |
| 10493 | GAGCTTCGGGTGCTCGCGCT | 245 |
| 10494 | AGCTTCGGGTGCTCGCGCTG | 246 |
| 10495 | GCTTCGGGTGCTCGCGCTGC | 247 |
| 10496 | CTTCGGGTGCTCGCGCTGCT | 248 |
| 10497 | TTCGGGTGCTCGCGCTGCTT | 249 |
| 10498 | TCGGGTGCTCGCGCTGCTTC | 250 |
| 10499 | CGGGTGCTCGCGCTGCTTCT | 251 |
| 10500 | GGGTGCTCGCGCTGCTTCTC | 252 |
| 10501 | GGTGCTCGCGCTGCTTCTCC | 253 |
| 10502 | GTGCTCGCGCTGCTTCTCCG | 254 |
| 10503 | TGCTCGCGCTGCTTCTCCGG | 255 |
| 10504 | GCTCGCGCTGCTTCTCCGGG | 256 |
| 10505 | CTCGCGCTGCTTCTCCGGGT | 257 |
| 10506 | TCGCGCTGCTTCTCCGGGTA | 258 |
| 10507 | CGCGCTGCTTCTCCGGGTAC | 259 |
| 10508 | GCGCTGCTTCTCCGGGTACG | 260 |
| 10509 | CGCTGCTTCTCCGGGTACGG | 261 |
| 10510 | GCTGCTTCTCCGGGTACGGG | 262 |
| 10511 | CTGCTTCTCCGGGTACGGGC | 263 |
| 10512 | TGCTTCTCCGGGTACGGGCC | 264 |
| 10513 | GCTTCTCCGGGTACGGGCCG | 265 |
| 10514 | CTTCTCCGGGTACGGGCCGC | 266 |
| 10515 | TTCTCCGGGTACGGGCCGCT | 267 |
| 10516 | TCTCCGGGTACGGGCCGCTG | 268 |
| 10517 | CTCCGGGTACGGGCCGCTGG | 269 |
| 10518 | TCCGGGTACGGGCCGCTGGG | 270 |
| 10519 | CCGGGTACGGGCCGCTGGGT | 271 |
| 10520 | CGGGTACGGGCCGCTGGGTG | 272 |
| 10521 | GGGTACGGGCCGCTGGGTGG | 273 |
| 10522 | GGTACGGGCCGCTGGGTGGG | 274 |
| 10523 | GTACGGGCCGCTGGGTGGGG | 275 |
| 10524 | TACGGGCCGCTGGGTGGGGT | 276 |
| 10525 | ACGGGCCGCTGGGTGGGGTC | 277 |
| 10526 | CGGGCCGCTGGGTGGGGTCC | 278 |
| 10527 | GGGCCGCTGGGTGGGGTCCC | 279 |
| 10528 | GGCCGCTGGGTGGGGTCCCG | 280 |
| 10529 | GCCGCTGGGTGGGGTCCCGG | 281 |
| 10530 | CCGCTGGGTGGGGTCCCGGG | 282 |
| 10531 | CGCTGGGTGGGGTCCCGGGC | 283 |
| 10532 | GCTGGGTGGGGTCCCGGGCG | 284 |
| 10533 | CTGGGTGGGGTCCCGGGCGT | 285 |
| 10534 | TGGGTGGGGTCCCGGGCGTG | 286 |
| 10535 | GGGTGGGGTCCCGGGCGTGG | 287 |
| 10536 | GGTGGGGTCCCGGGCGTGGT | 288 |
| 10537 | GTGGGGTCCCGGGCGTGGTG | 289 |
| 10538 | TGGGGTCCCGGGCGTGGTGC | 290 |
| 10539 | GGGGTCCCGGGCGTGGTGCG | 291 |
| 10540 | GGGTCCCGGGCGTGGTGCGG | 292 |
| 10541 | GGTCCCGGGCGTGGTGCGGA | 293 |
| 10542 | GTCCCGGGCGTGGTGCGGAG | 294 |
| 10543 | TCCCGGGCGTGGTGCGGAGG | 295 |
| 10544 | CCCGGGCGTGGTGCGGAGGC | 296 |
| 10545 | CCGGGCGTGGTGCGGAGGCG | 297 |
| 10546 | CGGGCGTGGTGCGGAGGCGC | 298 |
| 10547 | CCGCGCACTCGCCCGGCCCC | 200 |
| 10548 | GCCGCGCACTCGCCCGGCCC | 199 |
| 10549 | TGCCGCGCACTCGCCCGGCC | 198 |
| 10550 | ATGCCGCGCACTCGCCCGGC | 197 |
| 10551 | GATGCCGCGCACTCGCCCGG | 196 |
| 10552 | GGATGCCGCGCACTCGCCCG | 195 |
| 10553 | GGGATGCCGCGCACTCGCCC | 194 |
| 10554 | TGGGATGCCGCGCACTCGCC | 193 |
| 10555 | CTGGGATGCCGCGCACTCGC | 192 |
| 10556 | CCTGGGATGCCGCGCACTCG | 191 |
| 10557 | GCCTGGGATGCCGCGCACTC | 190 |
| 10558 | GGCCTGGGATGCCGCGCACT | 189 |
| 10559 | CGGCCTGGGATGCCGCGCAC | 188 |
| 10560 | CCGGCCTGGGATGCCGCGCA | 187 |
| 10561 | GCCGGCCTGGGATGCCGCGC | 186 |
| 10562 | GGCCGGCCTGGGATGCCGCG | 185 |
| 10563 | GGGCCGGCCTGGGATGCCGC | 184 |
| 10564 | CGGGCCGGCCTGGGATGCCG | 183 |
| 10565 | TCGGGCCGGCCTGGGATGCC | 182 |
| 10566 | TTCGGGCCGGCCTGGGATGC | 181 |
| 10567 | GTTCGGGCCGGCCTGGGATG | 180 |
| 10568 | CGTTCGGGCCGGCCTGGGAT | 179 |
| 10569 | GCGTTCGGGCCGGCCTGGGA | 178 |
| 10570 | AGCGTTCGGGCCGGCCTGGG | 177 |
| 10571 | GAGCGTTCGGGCCGGCCTGG | 176 |
| 10572 | GGAGCGTTCGGGCCGGCCTG | 175 |
| 10573 | CGGAGCGTTCGGGCCGGCCT | 174 |
| 10574 | GCGGAGCGTTCGGGCCGGCC | 173 |
| 10575 | GGCGGAGCGTTCGGGCCGGC | 172 |
| 10576 | GGGCGGAGCGTTCGGGCCGG | 171 |
| 10577 | CGGGCGGAGCGTTCGGGCCG | 170 |
| 10578 | GCGGGCGGAGCGTTCGGGCC | 169 |
| 10579 | CGCGGGCGGAGCGTTCGGGC | 168 |
| 10580 | CCGCGGGCGGAGCGTTCGGG | 167 |
| 10581 | ACCGCGGGCGGAGCGTTCGG | 166 |
| 10582 | CACCGCGGGCGGAGCGTTCG | 165 |
| 10583 | CCACCGCGGGCGGAGCGTTC | 164 |
| 10584 | CCCACCGCGGGCGGAGCGTT | 163 |
| 10585 | GCCCACCGCGGGCGGAGCGT | 162 |
| 10586 | GGCCCACCGCGGGCGGAGCG | 161 |
| 10587 | CGGCCCACCGCGGGCGGAGC | 160 |
| 10588 | TCGGCCCACCGCGGGCGGAG | 159 |
| 10589 | GTCGGCCCACCGCGGGCGGA | 158 |
| 10590 | AGTCGGCCCACCGCGGGCGG | 157 |
| 10591 | AAGTCGGCCCACCGCGGGCG | 156 |
| 10592 | GAAGTCGGCCCACCGCGGGC | 155 |
| 10593 | GGAAGTCGGCCCACCGCGGG | 154 |
| 10594 | GGGAAGTCGGCCCACCGCGG | 153 |
| 10595 | GGGGAAGTCGGCCCACCGCG | 152 |
| 10596 | AGGGGAAGTCGGCCCACCGC | 151 |
| 10597 | GAGGGGAAGTCGGCCCACCG | 150 |
| 10598 | GGAGGGGAAGTCGGCCCACC | 149 |
| 10599 | CGCCAGCCGGGAGCTTCGGGTGCTCGCG | 235 |
| 10600 | GCCAGCCGGGAGCTTCGGGT | 236 |
| 10601 | CCAGCCGGGAGCTTCGGGTG | 237 |
| 10602 | CAGCCGGGAGCTTCGGGTGC | 238 |
| 10603 | AGCCGGGAGCTTCGGGTGCT | 239 |
| 10604 | GCCGGGAGCTTCGGGTGCTC | 240 |
| 10605 | CCGGGAGCTTCGGGTGCTCG | 241 |
| 10606 | CGGGAGCTTCGGGTGCTCGC | 242 |
| 10607 | GGGAGCTTCGGGTGCTCGCG | 243 |
| 10608 | GGAGCTTCGGGTGCTCGCGC | 244 |
| 10609 | GAGCTTCGGGTGCTCGCGCT | 245 |
| 10610 | AGCTTCGGGTGCTCGCGCTG | 246 |
| 10611 | GCTTCGGGTGCTCGCGCTGC | 247 |
| 10612 | CTTCGGGTGCTCGCGCTGCT | 248 |
| 10613 | TTCGGGTGCTCGCGCTGCTT | 249 |
| 10614 | TCGGGTGCTCGCGCTGCTTC | 250 |
| 10615 | CGGGTGCTCGCGCTGCTTCT | 251 |
| 10616 | GGGTGCTCGCGCTGCTTCTC | 252 |
| 10617 | GGTGCTCGCGCTGCTTCTCC | 253 |
| 10618 | GTGCTCGCGCTGCTTCTCCG | 254 |
| 10619 | TGCTCGCGCTGCTTCTCCGG | 255 |
| 10620 | GCTCGCGCTGCTTCTCCGGG | 256 |
| 10621 | CTCGCGCTGCTTCTCCGGGT | 257 |
| 10622 | TCGCGCTGCTTCTCCGGGTA | 258 |
| 10623 | CGCGCTGCTTCTCCGGGTAC | 259 |
| 10624 | GCGCTGCTTCTCCGGGTACG | 260 |
| 10625 | CGCTGCTTCTCCGGGTACGG | 261 |
| 10626 | GCTGCTTCTCCGGGTACGGG | 262 |
| 10627 | CTGCTTCTCCGGGTACGGGC | 263 |
| 10628 | TGCTTCTCCGGGTACGGGCC | 264 |
| 10629 | GCTTCTCCGGGTACGGGCCG | 265 |
| 10630 | CTTCTCCGGGTACGGGCCGC | 266 |
| 10631 | TTCTCCGGGTACGGGCCGCT | 267 |
| 10632 | TCTCCGGGTACGGGCCGCTG | 268 |
| 10633 | CTCCGGGTACGGGCCGCTGG | 269 |
| 10634 | TCCGGGTACGGGCCGCTGGG | 270 |
| 10635 | CCGGGTACGGGCCGCTGGGT | 271 |
| 10636 | CGGGTACGGGCCGCTGGGTG | 272 |
| 10637 | GGGTACGGGCCGCTGGGTGG | 273 |
| 10638 | GGTACGGGCCGCTGGGTGGG | 274 |
| 10639 | GTACGGGCCGCTGGGTGGGG | 275 |
| 10640 | TACGGGCCGCTGGGTGGGGT | 276 |
| 10641 | ACGGGCCGCTGGGTGGGGTC | 277 |
| 10642 | CGGGCCGCTGGGTGGGGTCC | 278 |
| 10643 | GGGCCGCTGGGTGGGGTCCC | 279 |
| 10644 | GGCCGCTGGGTGGGGTCCCG | 280 |
| 10645 | GCCGCTGGGTGGGGTCCCGG | 281 |
| 10646 | CCGCTGGGTGGGGTCCCGGG | 282 |
| 10647 | CGCTGGGTGGGGTCCCGGGC | 283 |
| 10648 | GCTGGGTGGGGTCCCGGGCG | 284 |
| 10649 | CTGGGTGGGGTCCCGGGCGT | 285 |
| 10650 | TGGGTGGGGTCCCGGGCGTG | 286 |
| 10651 | GGGTGGGGTCCCGGGCGTGG | 287 |
| 10652 | GGTGGGGTCCCGGGCGTGGT | 288 |
| 10653 | GTGGGGTCCCGGGCGTGGTG | 289 |
| 10654 | TGGGGTCCCGGGCGTGGTGC | 290 |
| 10655 | GGGGTCCCGGGCGTGGTGCG | 291 |
| 10656 | GGGTCCCGGGCGTGGTGCGG | 292 |
| 10657 | GGTCCCGGGCGTGGTGCGGA | 293 |
| 10658 | GTCCCGGGCGTGGTGCGGAG | 294 |
| 10659 | TCCCGGGCGTGGTGCGGAGG | 295 |
| 10660 | CCCGGGCGTGGTGCGGAGGC | 296 |
| 10661 | CCGGGCGTGGTGCGGAGGCG | 297 |
| 10662 | CGGGCGTGGTGCGGAGGCGC | 298 |
| 10663 | CCGCCAGCCGGGAGCTTCGG | 234 |
| 10664 | GCCGCCAGCCGGGAGCTTCG | 233 |
| 10665 | TGCCGCCAGCCGGGAGCTTC | 232 |
| 10666 | CTGCCGCCAGCCGGGAGCTT | 231 |
| 10667 | TCTGCCGCCAGCCGGGAGCT | 230 |
| 10668 | TTCTGCCGCCAGCCGGGAGC | 229 |
| 10669 | TTTCTGCCGCCAGCCGGGAG | 228 |
| 10670 | GTTTCTGCCGCCAGCCGGGA | 227 |
| 10671 | GGTTTCTGCCGCCAGCCGGG | 226 |
| 10672 | CGGTTTCTGCCGCCAGCCGG | 225 |
| 10673 | CCGGTTTCTGCCGCCAGCCG | 224 |
| 10674 | CCCGGTTTCTGCCGCCAGCC | 223 |
| 10675 | TCCCGGTTTCTGCCGCCAGC | 222 |
| 10676 | CTCCCGGTTTCTGCCGCCAG | 221 |
| 10677 | ACTCCCGGTTTCTGCCGCCA | 220 |
| 10678 | CACTCCCGGTTTCTGCCGCC | 219 |
| 10679 | CCACTCCCGGTTTCTGCCGC | 218 |
| 10680 | CCCACTCCCGGTTTCTGCCG | 217 |
| 10681 | CCCCACTCCCGGTTTCTGCC | 216 |
| 10682 | GCCCCACTCCCGGTTTCTGC | 215 |
| 10683 | GGCCCCACTCCCGGTTTCTG | 214 |
| 10684 | CGGCCCCACTCCCGGTTTCT | 213 |
| 10685 | CCGGCCCCACTCCCGGTTTC | 212 |
| 10686 | CCCGGCCCCACTCCCGGTTT | 211 |
| 10687 | GCCCGGCCCCACTCCCGGTT | 210 |
| 10688 | CGCCCGGCCCCACTCCCGGT | 209 |
| 10689 | TCGCCCGGCCCCACTCCCGG | 208 |
| 10690 | CTCGCCCGGCCCCACTCCCG | 207 |
| 10691 | ACTCGCCCGGCCCCACTCCC | 206 |
| 10692 | CACTCGCCCGGCCCCACTCC | 205 |
| 10693 | GCACTCGCCCGGCCCCACTC | 204 |
| 10694 | CGCACTCGCCCGGCCCCACT | 203 |
| 10695 | GCGCACTCGCCCGGCCCCAC | 202 |
| 10696 | CGCGCACTCGCCCGGCCCCA | 201 |
| 10697 | CCGCGCACTCGCCCGGCCCC | 200 |
| 10698 | GCCGCGCACTCGCCCGGCCC | 199 |
| 10699 | TGCCGCGCACTCGCCCGGCC | 198 |
| 10700 | ATGCCGCGCACTCGCCCGGC | 197 |
| 10701 | GATGCCGCGCACTCGCCCGG | 196 |
| 10702 | GGATGCCGCGCACTCGCCCG | 195 |
| 10703 | GGGATGCCGCGCACTCGCCC | 194 |
| 10704 | TGGGATGCCGCGCACTCGCC | 193 |
| 10705 | CTGGGATGCCGCGCACTCGC | 192 |
| 10706 | CCTGGGATGCCGCGCACTCG | 191 |
| 10707 | GCCTGGGATGCCGCGCACTC | 190 |
| 10708 | GGCCTGGGATGCCGCGCACT | 189 |
| 10709 | CGGCCTGGGATGCCGCGCAC | 188 |
| 10710 | CCGGCCTGGGATGCCGCGCA | 187 |
| 10711 | GCCGGCCTGGGATGCCGCGC | 186 |
| 10712 | GGCCGGCCTGGGATGCCGCG | 185 |
| 10713 | GGGCCGGCCTGGGATGCCGC | 184 |
| 10714 | CGGGCCGGCCTGGGATGCCG | 183 |
| 10715 | TCGGGCCGGCCTGGGATGCC | 182 |
| 10716 | TTCGGGCCGGCCTGGGATGC | 181 |
| 10717 | GTTCGGGCCGGCCTGGGATG | 180 |
| 10718 | CGTTCGGGCCGGCCTGGGAT | 179 |
| 10719 | GCGTTCGGGCCGGCCTGGGA | 178 |
| 10720 | AGCGTTCGGGCCGGCCTGGG | 177 |
| 10721 | GAGCGTTCGGGCCGGCCTGG | 176 |
| 10722 | GGAGCGTTCGGGCCGGCCTG | 175 |
| 10723 | CGGAGCGTTCGGGCCGGCCT | 174 |
| 10724 | GCGGAGCGTTCGGGCCGGCC | 173 |
| 10725 | GGCGGAGCGTTCGGGCCGGC | 172 |
| 10726 | GGGCGGAGCGTTCGGGCCGG | 171 |
| 10727 | CGGGCGGAGCGTTCGGGCCG | 170 |
| 10728 | GCGGGCGGAGCGTTCGGGCC | 169 |
| 10729 | CGCGGGCGGAGCGTTCGGGC | 168 |
| 10730 | CCGCGGGCGGAGCGTTCGGG | 167 |
| 10731 | ACCGCGGGCGGAGCGTTCGG | 166 |
| 10732 | CACCGCGGGCGGAGCGTTCG | 165 |
| 10733 | CCACCGCGGGCGGAGCGTTC | 164 |
| 10734 | CCCACCGCGGGCGGAGCGTT | 163 |
| 10735 | GCCCACCGCGGGCGGAGCGT | 162 |
| 10736 | GGCCCACCGCGGGCGGAGCG | 161 |
| 10737 | CGGCCCACCGCGGGCGGAGC | 160 |
| 10738 | TCGGCCCACCGCGGGCGGAG | 159 |
| 10739 | GTCGGCCCACCGCGGGCGGA | 158 |
| 10740 | AGTCGGCCCACCGCGGGCGG | 157 |
| 10741 | AAGTCGGCCCACCGCGGGCG | 156 |
| 10742 | GAAGTCGGCCCACCGCGGGC | 155 |
| 10743 | GGAAGTCGGCCCACCGCGGG | 154 |
| 10744 | GGGAAGTCGGCCCACCGCGG | 153 |
| 10745 | GGGGAAGTCGGCCCACCGCG | 152 |
| 10746 | AGGGGAAGTCGGCCCACCGC | 151 |
| 10747 | GAGGGGAAGTCGGCCCACCG | 150 |
| 10748 | GGAGGGGAAGTCGGCCCACC | 149 |
| 10749 | CGGGTACGGGCCGCTGGGTGGGGTCCCG | 272 |
| 10750 | GGGTACGGGCCGCTGGGTGG | 273 |
| 10751 | GGTACGGGCCGCTGGGTGGG | 274 |
| 10752 | GTACGGGCCGCTGGGTGGGG | 275 |
| 10753 | TACGGGCCGCTGGGTGGGGT | 276 |
| 10754 | ACGGGCCGCTGGGTGGGGTC | 277 |
| 10755 | CGGGCCGCTGGGTGGGGTCC | 278 |
| 10756 | GGGCCGCTGGGTGGGGTCCC | 279 |
| 10757 | GGCCGCTGGGTGGGGTCCCG | 280 |
| 10758 | GCCGCTGGGTGGGGTCCCGG | 281 |
| 10759 | CCGCTGGGTGGGGTCCCGGG | 282 |
| 10760 | CGCTGGGTGGGGTCCCGGGC | 283 |
| 10761 | GCTGGGTGGGGTCCCGGGCG | 284 |
| 10762 | CTGGGTGGGGTCCCGGGCGT | 285 |
| 10763 | TGGGTGGGGTCCCGGGCGTG | 286 |
| 10764 | GGGTGGGGTCCCGGGCGTGG | 287 |
| 10765 | GGTGGGGTCCCGGGCGTGGT | 288 |
| 10766 | GTGGGGTCCCGGGCGTGGTG | 289 |
| 10767 | TGGGGTCCCGGGCGTGGTGC | 290 |
| 10768 | GGGGTCCCGGGCGTGGTGCG | 291 |
| 10769 | GGGTCCCGGGCGTGGTGCGG | 292 |
| 10770 | GGTCCCGGGCGTGGTGCGGA | 293 |
| 10771 | GTCCCGGGCGTGGTGCGGAG | 294 |
| 10772 | TCCCGGGCGTGGTGCGGAGG | 295 |
| 10773 | CCCGGGCGTGGTGCGGAGGC | 296 |
| 10774 | CCGGGCGTGGTGCGGAGGCG | 297 |
| 10775 | CGGGCGTGGTGCGGAGGCGC | 298 |
| 10776 | CCGGGTACGGGCCGCTGGGT | 271 |
| 10777 | TCCGGGTACGGGCCGCTGGG | 270 |
| 10778 | CTCCGGGTACGGGCCGCTGG | 269 |
| 10779 | TCTCCGGGTACGGGCCGCTG | 268 |
| 10780 | TTCTCCGGGTACGGGCCGCT | 267 |
| 10781 | CTTCTCCGGGTACGGGCCGC | 266 |
| 10782 | GCTTCTCCGGGTACGGGCCG | 265 |
| 10783 | TGCTTCTCCGGGTACGGGCC | 264 |
| 10784 | CTGCTTCTCCGGGTACGGGC | 263 |
| 10785 | GCTGCTTCTCCGGGTACGGG | 262 |
| 10786 | CGCTGCTTCTCCGGGTACGG | 261 |
| 10787 | GCGCTGCTTCTCCGGGTACG | 260 |
| 10788 | CGCGCTGCTTCTCCGGGTAC | 259 |
| 10789 | TCGCGCTGCTTCTCCGGGTA | 258 |
| 10790 | CTCGCGCTGCTTCTCCGGGT | 257 |
| 10791 | GCTCGCGCTGCTTCTCCGGG | 256 |
| 10792 | TGCTCGCGCTGCTTCTCCGG | 255 |
| 10793 | GTGCTCGCGCTGCTTCTCCG | 254 |
| 10794 | GGTGCTCGCGCTGCTTCTCC | 253 |
| 10795 | GGGTGCTCGCGCTGCTTCTC | 252 |
| 10796 | CGGGTGCTCGCGCTGCTTCT | 251 |
| 10797 | TCGGGTGCTCGCGCTGCTTC | 250 |
| 10798 | TTCGGGTGCTCGCGCTGCTT | 249 |
| 10799 | CTTCGGGTGCTCGCGCTGCT | 248 |
| 10800 | GCTTCGGGTGCTCGCGCTGC | 247 |
| 10801 | AGCTTCGGGTGCTCGCGCTG | 246 |
| 10802 | GAGCTTCGGGTGCTCGCGCT | 245 |
| 10803 | GGAGCTTCGGGTGCTCGCGC | 244 |
| 10804 | GGGAGCTTCGGGTGCTCGCG | 243 |
| 10805 | CGGGAGCTTCGGGTGCTCGC | 242 |
| 10806 | CCGGGAGCTTCGGGTGCTCG | 241 |
| 10807 | GCCGGGAGCTTCGGGTGCTC | 240 |
| 10808 | AGCCGGGAGCTTCGGGTGCT | 239 |
| 10809 | CAGCCGGGAGCTTCGGGTGC | 238 |
| 10810 | CCAGCCGGGAGCTTCGGGTG | 237 |
| 10811 | GCCAGCCGGGAGCTTCGGGT | 236 |
| 10812 | CGCCAGCCGGGAGCTTCGGG | 235 |
| 10813 | CCGCCAGCCGGGAGCTTCGG | 234 |
| 10814 | GCCGCCAGCCGGGAGCTTCG | 233 |
| 10815 | TGCCGCCAGCCGGGAGCTTC | 232 |
| 10816 | CTGCCGCCAGCCGGGAGCTT | 231 |
| 10817 | TCTGCCGCCAGCCGGGAGCT | 230 |
| 10818 | TTCTGCCGCCAGCCGGGAGC | 229 |
| 10819 | TTTCTGCCGCCAGCCGGGAG | 228 |
| 10820 | GTTTCTGCCGCCAGCCGGGA | 227 |
| 10821 | GGTTTCTGCCGCCAGCCGGG | 226 |
| 10822 | CGGTTTCTGCCGCCAGCCGG | 225 |
| 10823 | CCGGTTTCTGCCGCCAGCCG | 224 |
| 10824 | CCCGGTTTCTGCCGCCAGCC | 223 |
| 10825 | TCCCGGTTTCTGCCGCCAGC | 222 |
| 10826 | CTCCCGGTTTCTGCCGCCAG | 221 |
| 10827 | ACTCCCGGTTTCTGCCGCCA | 220 |
| 10828 | CACTCCCGGTTTCTGCCGCC | 219 |
| 10829 | CCACTCCCGGTTTCTGCCGC | 218 |
| 10830 | CCCACTCCCGGTTTCTGCCG | 217 |
| 10831 | CCCCACTCCCGGTTTCTGCC | 216 |
| 10832 | GCCCCACTCCCGGTTTCTGC | 215 |
| 10833 | GGCCCCACTCCCGGTTTCTG | 214 |
| 10834 | CGGCCCCACTCCCGGTTTCT | 213 |
| 10835 | CCGGCCCCACTCCCGGTTTC | 212 |
| 10836 | CCCGGCCCCACTCCCGGTTT | 211 |
| 10837 | GCCCGGCCCCACTCCCGGTT | 210 |
| 10838 | CGCCCGGCCCCACTCCCGGT | 209 |
| 10839 | TCGCCCGGCCCCACTCCCGG | 208 |
| 10840 | CTCGCCCGGCCCCACTCCCG | 207 |
| 10841 | ACTCGCCCGGCCCCACTCCC | 206 |
| 10842 | CACTCGCCCGGCCCCACTCC | 205 |
| 10843 | GCACTCGCCCGGCCCCACTC | 204 |
| 10844 | CGCACTCGCCCGGCCCCACT | 203 |
| 10845 | GCGCACTCGCCCGGCCCCAC | 202 |
| 10846 | CGCGCACTCGCCCGGCCCCA | 201 |
| 10847 | CCGCGCACTCGCCCGGCCCC | 200 |
| 10848 | GCCGCGCACTCGCCCGGCCC | 199 |
| 10849 | TGCCGCGCACTCGCCCGGCC | 198 |
| 10850 | ATGCCGCGCACTCGCCCGGC | 197 |
| 10851 | GATGCCGCGCACTCGCCCGG | 196 |
| 10852 | GGATGCCGCGCACTCGCCCG | 195 |
| 10853 | GGGATGCCGCGCACTCGCCC | 194 |
| 10854 | TGGGATGCCGCGCACTCGCC | 193 |
| 10855 | CTGGGATGCCGCGCACTCGC | 192 |
| 10856 | CCTGGGATGCCGCGCACTCG | 191 |
| 10857 | GCCTGGGATGCCGCGCACTC | 190 |
| 10858 | GGCCTGGGATGCCGCGCACT | 189 |
| 10859 | CGGCCTGGGATGCCGCGCAC | 188 |
| 10860 | CCGGCCTGGGATGCCGCGCA | 187 |
| 10861 | GCCGGCCTGGGATGCCGCGC | 186 |
| 10862 | GGCCGGCCTGGGATGCCGCG | 185 |
| 10863 | GGGCCGGCCTGGGATGCCGC | 184 |
| 10864 | CGGGCCGGCCTGGGATGCCG | 183 |
| 10865 | TCGGGCCGGCCTGGGATGCC | 182 |
| 10866 | TTCGGGCCGGCCTGGGATGC | 181 |
| 10867 | GTTCGGGCCGGCCTGGGATG | 180 |
| 10868 | CGTTCGGGCCGGCCTGGGAT | 179 |
| 10869 | GCGTTCGGGCCGGCCTGGGA | 178 |
| 10870 | AGCGTTCGGGCCGGCCTGGG | 177 |
| 10871 | GAGCGTTCGGGCCGGCCTGG | 176 |
| 10872 | GGAGCGTTCGGGCCGGCCTG | 175 |
| 10873 | CGGAGCGTTCGGGCCGGCCT | 174 |
| 10874 | GCGGAGCGTTCGGGCCGGCC | 173 |
| 10875 | GGCGGAGCGTTCGGGCCGGC | 172 |
| 10876 | GGGCGGAGCGTTCGGGCCGG | 171 |
| 10877 | CGGGCGGAGCGTTCGGGCCG | 170 |
| 10878 | GCGGGCGGAGCGTTCGGGCC | 169 |
| 10879 | CGCGGGCGGAGCGTTCGGGC | 168 |
| 10880 | CCGCGGGCGGAGCGTTCGGG | 167 |
| 10881 | ACCGCGGGCGGAGCGTTCGG | 166 |
| 10882 | CACCGCGGGCGGAGCGTTCG | 165 |
| 10883 | CCACCGCGGGCGGAGCGTTC | 164 |
| 10884 | CCCACCGCGGGCGGAGCGTT | 163 |
| 10885 | GCCCACCGCGGGCGGAGCGT | 162 |
| 10886 | GGCCCACCGCGGGCGGAGCG | 161 |
| 10887 | CGGCCCACCGCGGGCGGAGC | 160 |
| 10888 | TCGGCCCACCGCGGGCGGAG | 159 |
| 10889 | GTCGGCCCACCGCGGGCGGA | 158 |
| 10890 | AGTCGGCCCACCGCGGGCGG | 157 |
| 10891 | AAGTCGGCCCACCGCGGGCG | 156 |
| 10892 | GAAGTCGGCCCACCGCGGGC | 155 |
| 10893 | GGAAGTCGGCCCACCGCGGG | 154 |
| 10894 | GGGAAGTCGGCCCACCGCGG | 153 |
| 10895 | GGGGAAGTCGGCCCACCGCG | 152 |
| 10896 | AGGGGAAGTCGGCCCACCGC | 151 |
| 10897 | GAGGGGAAGTCGGCCCACCG | 150 |
| 10898 | GGAGGGGAAGTCGGCCCACC | 149 |
| 10899 | GTGCGGAGGCGCAGGGCCGGGCTCCG | 306 |
| 10900 | CTACGCGCGGCTGCAGGGGGCGC | 339 |
| 10901 | TACGCGCGGCTGCAGGGGGC | 340 |
| 10902 | ACGCGCGGCTGCAGGGGGCG | 341 |
| 10903 | CGCGCGGCTGCAGGGGGCGC | 342 |
| 10904 | GCGCGGCTGCAGGGGGCGCT | 343 |
| 10905 | CGCGGCTGCAGGGGGCGCTG | 344 |
| 10906 | GCGGCTGCAGGGGGCGCTGG | 345 |
| 10907 | CGGCTGCAGGGGGCGCTGGG | 346 |
| 10908 | CCTACGCGCGGCTGCAGGGG | 338 |
| 10909 | GCCTACGCGCGGCTGCAGGG | 337 |
| 10910 | TGCCTACGCGCGGCTGCAGG | 336 |
| 10911 | CTGCCTACGCGCGGCTGCAG | 335 |
| 10912 | TCTGCCTACGCGCGGCTGCA | 334 |
| 10913 | CTCTGCCTACGCGCGGCTGC | 333 |
| 10914 | TCTCTGCCTACGCGCGGCTG | 332 |
| 10915 | GTCTCTGCCTACGCGCGGCT | 331 |
| 10916 | CGTCTCTGCCTACGCGCGGC | 330 |
| 10917 | CCGTCTCTGCCTACGCGCGG | 329 |
| 10918 | TCCGTCTCTGCCTACGCGCG | 328 |
| 10919 | CTCCGTCTCTGCCTACGCGC | 327 |
| 10920 | GCTCCGTCTCTGCCTACGCG | 326 |
| 10921 | GGCTCCGTCTCTGCCTACGC | 325 |
| 10922 | GGGCTCCGTCTCTGCCTACG | 324 |
| 10923 | CGGGCTCCGTCTCTGCCTAC | 323 |
| 10924 | CCGGGCTCCGTCTCTGCCTA | 322 |
| 10925 | GCCGGGCTCCGTCTCTGCCT | 321 |
| 10926 | GGCCGGGCTCCGTCTCTGCC | 320 |
| 10927 | GGGCCGGGCTCCGTCTCTGC | 319 |
| 10928 | AGGGCCGGGCTCCGTCTCTG | 318 |
| 10929 | CAGGGCCGGGCTCCGTCTCT | 317 |
| 10930 | GCAGGGCCGGGCTCCGTCTC | 316 |
| 10931 | CGCAGGGCCGGGCTCCGTCT | 315 |
| 10932 | GCGCAGGGCCGGGCTCCGTC | 314 |
| 10933 | GGCGCAGGGCCGGGCTCCGT | 313 |
| 10934 | AGGCGCAGGGCCGGGCTCCG | 312 |
| 10935 | GAGGCGCAGGGCCGGGCTCC | 311 |
| 10936 | GGAGGCGCAGGGCCGGGCTC | 310 |
| 10937 | CGGAGGCGCAGGGCCGGGCT | 309 |
| 10938 | CTGCGCAGGACTCGCGTCCTGGCCCG | 375 |
| 10939 | TGCGCAGGACTCGCGTCCTG | 376 |
| 10940 | GCGCAGGACTCGCGTCCTGG | 377 |
| 10941 | CGCAGGACTCGCGTCCTGGC | 378 |
| 10942 | GCAGGACTCGCGTCCTGGCC | 379 |
| 10943 | CAGGACTCGCGTCCTGGCCC | 380 |
| 10944 | AGGACTCGCGTCCTGGCCCG | 381 |
| 10945 | GGACTCGCGTCCTGGCCCGG | 382 |
| 10946 | GACTCGCGTCCTGGCCCGGG | 383 |
| 10947 | ACTCGCGTCCTGGCCCGGGC | 384 |
| 10948 | CTCGCGTCCTGGCCCGGGCC | 385 |
| 10949 | TCGCGTCCTGGCCCGGGCCT | 386 |
| 10950 | CGCGTCCTGGCCCGGGCCTC | 387 |
| 10951 | GCGTCCTGGCCCGGGCCTCC | 388 |
| 10952 | CGTCCTGGCCCGGGCCTCCC | 389 |
| 10953 | GTCCTGGCCCGGGCCTCCCA | 390 |
| 10954 | TCCTGGCCCGGGCCTCCCAG | 391 |
| 10955 | CCTGGCCCGGGCCTCCCAGC | 392 |
| 10956 | CTGGCCCGGGCCTCCCAGCC | 393 |
| 10957 | TGGCCCGGGCCTCCCAGCCC | 394 |
| 10958 | GGCCCGGGCCTCCCAGCCCG | 395 |
| 10959 | GCCCGGGCCTCCCAGCCCGC | 396 |
| 10960 | CCCGGGCCTCCCAGCCCGCA | 397 |
| 10961 | CCGGGCCTCCCAGCCCGCAG | 398 |
| 10962 | CGGGCCTCCCAGCCCGCAGA | 399 |
| 10963 | GGGCCTCCCAGCCCGCAGAG | 400 |
| 10964 | GGCCTCCCAGCCCGCAGAGC | 401 |
| 10965 | GCCTCCCAGCCCGCAGAGCG | 402 |
| 10966 | CCTCCCAGCCCGCAGAGCGC | 403 |
| 10967 | CTCCCAGCCCGCAGAGCGCG | 404 |
| 10968 | TCCCAGCCCGCAGAGCGCGG | 405 |
| 10969 | CCCAGCCCGCAGAGCGCGGG | 406 |
| 10970 | CCAGCCCGCAGAGCGCGGGA | 407 |
| 10971 | CAGCCCGCAGAGCGCGGGAT | 408 |
| 10972 | AGCCCGCAGAGCGCGGGATG | 409 |
| 10973 | GCCCGCAGAGCGCGGGATGG | 410 |
| 10974 | CCCGCAGAGCGCGGGATGGC | 411 |
| 10975 | CCGCAGAGCGCGGGATGGCT | 412 |
| 10976 | CGCAGAGCGCGGGATGGCTC | 413 |
| 10977 | GCAGAGCGCGGGATGGCTCT | 414 |
| 10978 | CAGAGCGCGGGATGGCTCTG | 415 |
| 10979 | AGAGCGCGGGATGGCTCTGG | 416 |
| 10980 | GAGCGCGGGATGGCTCTGGG | 417 |
| 10981 | AGCGCGGGATGGCTCTGGGC | 418 |
| 10982 | GCGCGGGATGGCTCTGGGCT | 419 |
| 10983 | CGCGGGATGGCTCTGGGCTC | 420 |
| 10984 | GCGGGATGGCTCTGGGCTCA | 421 |
| 10985 | CGGGATGGCTCTGGGCTCAG | 422 |
| 10986 | GCTGCGCAGGACTCGCGTCC | 374 |
| 10987 | GGCTGCGCAGGACTCGCGTC | 373 |
| 10988 | CGGCTGCGCAGGACTCGCGT | 372 |
| 10989 | TCGGCTGCGCAGGACTCGCG | 371 |
| 10990 | CTCGGCTGCGCAGGACTCGC | 370 |
| 10991 | CCTCGGCTGCGCAGGACTCG | 369 |
| 10992 | ACCTCGGCTGCGCAGGACTC | 368 |
| 10993 | AACCTCGGCTGCGCAGGACT | 367 |
| 10994 | GAACCTCGGCTGCGCAGGAC | 366 |
| 10995 | GGAACCTCGGCTGCGCAGGA | 365 |
| 10996 | GGGAACCTCGGCTGCGCAGG | 364 |
| 10997 | GGGGAACCTCGGCTGCGCAG | 363 |
| 10998 | TGGGGAACCTCGGCTGCGCA | 362 |
| 10999 | CTGGGGAACCTCGGCTGCGC | 361 |
| 11000 | GCTGGGGAACCTCGGCTGCG | 360 |
| 11001 | CGCTGGGGAACCTCGGCTGC | 359 |
| 11002 | GCGCTGGGGAACCTCGGCTG | 358 |
| 11003 | GGCGCTGGGGAACCTCGGCT | 357 |
| 11004 | GGGCGCTGGGGAACCTCGGC | 356 |
| 11005 | GGGGCGCTGGGGAACCTCGG | 355 |
| 11006 | GGGGGCGCTGGGGAACCTCG | 354 |
| 11007 | AGGGGGCGCTGGGGAACCTC | 353 |
| 11008 | CAGGGGGCGCTGGGGAACCT | 352 |
| 11009 | CCCGCAGAGCGCGGGATGGCTC | 411 |
| 11010 | CCGCAGAGCGCGGGATGGCT | 412 |
| 11011 | CGCAGAGCGCGGGATGGCTC | 413 |
| 11012 | GCAGAGCGCGGGATGGCTCT | 414 |
| 11013 | CAGAGCGCGGGATGGCTCTG | 415 |
| 11014 | AGAGCGCGGGATGGCTCTGG | 416 |
| 11015 | GAGCGCGGGATGGCTCTGGG | 417 |
| 11016 | AGCGCGGGATGGCTCTGGGC | 418 |
| 11017 | GCGCGGGATGGCTCTGGGCT | 419 |
| 11018 | CGCGGGATGGCTCTGGGCTC | 420 |
| 11019 | GCGGGATGGCTCTGGGCTCA | 421 |
| 11020 | CGGGATGGCTCTGGGCTCAG | 422 |
| 11021 | GCCCGCAGAGCGCGGGATGG | 410 |
| 11022 | AGCCCGCAGAGCGCGGGATG | 409 |
| 11023 | CAGCCCGCAGAGCGCGGGAT | 408 |
| 11024 | CCAGCCCGCAGAGCGCGGGA | 407 |
| 11025 | CCCAGCCCGCAGAGCGCGGG | 406 |
| 11026 | TCCCAGCCCGCAGAGCGCGG | 405 |
| 11027 | CTCCCAGCCCGCAGAGCGCG | 404 |
| 11028 | CCTCCCAGCCCGCAGAGCGC | 403 |
| 11029 | GCCTCCCAGCCCGCAGAGCG | 402 |
| 11030 | GGCCTCCCAGCCCGCAGAGC | 401 |
| 11031 | GGGCCTCCCAGCCCGCAGAG | 400 |
| 11032 | CGGGCCTCCCAGCCCGCAGA | 399 |
| 11033 | CCGGGCCTCCCAGCCCGCAG | 398 |
| 11034 | CCCGGGCCTCCCAGCCCGCA | 397 |
| 11035 | GCCCGGGCCTCCCAGCCCGC | 396 |
| 11036 | GGCCCGGGCCTCCCAGCCCG | 395 |
| 11037 | TGGCCCGGGCCTCCCAGCCC | 394 |
| 11038 | CTGGCCCGGGCCTCCCAGCC | 393 |
| 11039 | CCTGGCCCGGGCCTCCCAGC | 392 |
| 11040 | TCCTGGCCCGGGCCTCCCAG | 391 |
| 11041 | GTCCTGGCCCGGGCCTCCCA | 390 |
| 11042 | CGTCCTGGCCCGGGCCTCCC | 389 |
| 11043 | GCGTCCTGGCCCGGGCCTCC | 388 |
| 11044 | CGCGTCCTGGCCCGGGCCTC | 387 |
| 11045 | TCGCGTCCTGGCCCGGGCCT | 386 |
| 11046 | CTCGCGTCCTGGCCCGGGCC | 385 |
| 11047 | ACTCGCGTCCTGGCCCGGGC | 384 |
| 11048 | GACTCGCGTCCTGGCCCGGG | 383 |
| 11049 | GGACTCGCGTCCTGGCCCGG | 382 |
| 11050 | AGGACTCGCGTCCTGGCCCG | 381 |
| 11051 | CAGGACTCGCGTCCTGGCCC | 380 |
| 11052 | GCAGGACTCGCGTCCTGGCC | 379 |
| 11053 | CGCAGGACTCGCGTCCTGGC | 378 |
| 11054 | GCGCAGGACTCGCGTCCTGG | 377 |
| 11055 | TGCGCAGGACTCGCGTCCTG | 376 |
| 11056 | CTGCGCAGGACTCGCGTCCT | 375 |
| 11057 | GCTGCGCAGGACTCGCGTCC | 374 |
| 11058 | GGCTGCGCAGGACTCGCGTC | 373 |
| 11059 | CGGCTGCGCAGGACTCGCGT | 372 |
| 11060 | TCGGCTGCGCAGGACTCGCG | 371 |
| 11061 | CTCGGCTGCGCAGGACTCGC | 370 |
| 11062 | CCTCGGCTGCGCAGGACTCG | 369 |
| 11063 | ACCTCGGCTGCGCAGGACTC | 368 |
| 11064 | AACCTCGGCTGCGCAGGACT | 367 |
| 11065 | GAACCTCGGCTGCGCAGGAC | 366 |
| 11066 | GGAACCTCGGCTGCGCAGGA | 365 |
| 11067 | GGGAACCTCGGCTGCGCAGG | 364 |
| 11068 | GGGGAACCTCGGCTGCGCAG | 363 |
| 11069 | TGGGGAACCTCGGCTGCGCA | 362 |
| 11070 | CTGGGGAACCTCGGCTGCGC | 361 |
| 11071 | GCTGGGGAACCTCGGCTGCG | 360 |
| 11072 | CGCTGGGGAACCTCGGCTGC | 359 |
| 11073 | GCGCTGGGGAACCTCGGCTG | 358 |
| 11074 | GGCGCTGGGGAACCTCGGCT | 357 |
| 11075 | GGGCGCTGGGGAACCTCGGC | 356 |
| 11076 | GGGGCGCTGGGGAACCTCGG | 355 |
| 11077 | GGGGGCGCTGGGGAACCTCG | 354 |
| 11078 | AGGGGGCGCTGGGGAACCTC | 353 |
| 11079 | CAGGGGGCGCTGGGGAACCT | 352 |
| 11080 | CGGACCTGATGGGGCACGGGCTTCCCC | 448 |
| 11081 | GGACCTGATGGGGCACGGGC | 449 |
| 11082 | GACCTGATGGGGCACGGGCT | 450 |
| 11083 | ACCTGATGGGGCACGGGCTT | 451 |
| 11084 | CCTGATGGGGCACGGGCTTC | 452 |
| 11085 | CTGATGGGGCACGGGCTTCC | 453 |
| 11086 | TGATGGGGCACGGGCTTCCC | 454 |
| 11087 | GATGGGGCACGGGCTTCCCC | 455 |
| 11088 | ATGGGGCACGGGCTTCCCCT | 456 |
| 11089 | TGGGGCACGGGCTTCCCCTT | 457 |
| 11090 | GGGGCACGGGCTTCCCCTTT | 458 |
| 11091 | GGGCACGGGCTTCCCCTTTT | 459 |
| 11092 | GGCACGGGCTTCCCCTTTTA | 460 |
| 11093 | GCACGGGCTTCCCCTTTTAA | 461 |
| 11094 | CACGGGCTTCCCCTTTTAAC | 462 |
| 11095 | ACGGGCTTCCCCTTTTAACG | 463 |
| 11096 | CGGGCTTCCCCTTTTAACGG | 464 |
| 11097 | GGGCTTCCCCTTTTAACGGT | 465 |
| 11098 | GGCTTCCCCTTTTAACGGTG | 466 |
| 11099 | GCTTCCCCTTTTAACGGTGG | 467 |
| 11100 | CTTCCCCTTTTAACGGTGGT | 468 |
| 11101 | TTCCCCTTTTAACGGTGGTT | 469 |
| 11102 | TCCCCTTTTAACGGTGGTTG | 470 |
| 11103 | CCCCTTTTAACGGTGGTTGG | 471 |
| 11104 | CCCTTTTAACGGTGGTTGGG | 472 |
| 11105 | CCTTTTAACGGTGGTTGGGG | 473 |
| 11106 | CTTTTAACGGTGGTTGGGGC | 474 |
| 11107 | TTTTAACGGTGGTTGGGGCC | 475 |
| 11108 | TTTAACGGTGGTTGGGGCCT | 476 |
| 11109 | TTAACGGTGGTTGGGGCCTA | 477 |
| 11110 | TAACGGTGGTTGGGGCCTAG | 478 |
| 11111 | AACGGTGGTTGGGGCCTAGA | 479 |
| 11112 | ACGGTGGTTGGGGCCTAGAA | 480 |
| 11113 | CGGTGGTTGGGGCCTAGAAG | 481 |
| 11114 | GCGGACCTGATGGGGCACGG | 447 |
| 11115 | AGCGGACCTGATGGGGCACG | 446 |
| 11116 | GAGCGGACCTGATGGGGCAC | 445 |
| 11117 | CGGTGGTTGGGGCCTAGAAGCG | 481 |
| 11118 | ACGGTGGTTGGGGCCTAGAA | 480 |
| 11119 | AACGGTGGTTGGGGCCTAGA | 479 |
| 11120 | TAACGGTGGTTGGGGCCTAG | 478 |
| 11121 | TTAACGGTGGTTGGGGCCTA | 477 |
| 11122 | TTTAACGGTGGTTGGGGCCT | 476 |
| 11123 | TTTTAACGGTGGTTGGGGCC | 475 |
| 11124 | CTTTTAACGGTGGTTGGGGC | 474 |
| 11125 | CCTTTTAACGGTGGTTGGGG | 473 |
| 11126 | CCCTTTTAACGGTGGTTGGG | 472 |
| 11127 | CCCCTTTTAACGGTGGTTGG | 471 |
| 11128 | TCCCCTTTTAACGGTGGTTG | 470 |
| 11129 | TTCCCCTTTTAACGGTGGTT | 469 |
| 11130 | CTTCCCCTTTTAACGGTGGT | 468 |
| 11131 | GCTTCCCCTTTTAACGGTGG | 467 |
| 11132 | GGCTTCCCCTTTTAACGGTG | 466 |
| 11133 | GGGCTTCCCCTTTTAACGGT | 465 |
| 11134 | CGGGCTTCCCCTTTTAACGG | 464 |
| 11135 | ACGGGCTTCCCCTTTTAACG | 463 |
| 11136 | CACGGGCTTCCCCTTTTAAC | 462 |
| 11137 | GCACGGGCTTCCCCTTTTAA | 461 |
| 11138 | GGCACGGGCTTCCCCTTTTA | 460 |
| 11139 | GGGCACGGGCTTCCCCTTTT | 459 |
| 11140 | GGGGCACGGGCTTCCCCTTT | 458 |
| 11141 | TGGGGCACGGGCTTCCCCTT | 457 |
| 11142 | ATGGGGCACGGGCTTCCCCT | 456 |
| 11143 | GATGGGGCACGGGCTTCCCC | 455 |
| 11144 | TGATGGGGCACGGGCTTCCC | 454 |
| 11145 | CTGATGGGGCACGGGCTTCC | 453 |
| 11146 | CCTGATGGGGCACGGGCTTC | 452 |
| 11147 | ACCTGATGGGGCACGGGCTT | 451 |
| 11148 | GACCTGATGGGGCACGGGCT | 450 |
| 11149 | GGACCTGATGGGGCACGGGC | 449 |
| 11150 | CGGACCTGATGGGGCACGGG | 448 |
| 11151 | GCGGACCTGATGGGGCACGG | 447 |
| 11152 | AGCGGACCTGATGGGGCACG | 446 |
| 11153 | GAGCGGACCTGATGGGGCAC | 445 |
| 11154 | CGCGCCCCTCGCTGTGACCGCCCAGCCCG | 524 |
| 11155 | GCGCCCCTCGCTGTGACCGC | 525 |
| 11156 | CGCCCCTCGCTGTGACCGCC | 526 |
| 11157 | GCCCCTCGCTGTGACCGCCC | 527 |
| 11158 | CCCCTCGCTGTGACCGCCCA | 528 |
| 11159 | CCCTCGCTGTGACCGCCCAG | 529 |
| 11160 | CCTCGCTGTGACCGCCCAGC | 530 |
| 11161 | CTCGCTGTGACCGCCCAGCC | 531 |
| 11162 | TCGCTGTGACCGCCCAGCCC | 532 |
| 11163 | CGCTGTGACCGCCCAGCCCG | 533 |
| 11164 | GCTGTGACCGCCCAGCCCGG | 534 |
| 11165 | CTGTGACCGCCCAGCCCGGC | 535 |
| 11166 | TGTGACCGCCCAGCCCGGCG | 536 |
| 11167 | GTGACCGCCCAGCCCGGCGT | 537 |
| 11168 | TGACCGCCCAGCCCGGCGTG | 538 |
| 11169 | GACCGCCCAGCCCGGCGTGG | 539 |
| 11170 | ACCGCCCAGCCCGGCGTGGC | 540 |
| 11171 | CCGCCCAGCCCGGCGTGGCC | 541 |
| 11172 | CGCCCAGCCCGGCGTGGCCC | 542 |
| 11173 | GCCCAGCCCGGCGTGGCCCA | 543 |
| 11174 | CCCAGCCCGGCGTGGCCCAA | 544 |
| 11175 | CCAGCCCGGCGTGGCCCAAA | 545 |
| 11176 | CAGCCCGGCGTGGCCCAAAT | 546 |
| 11177 | AGCCCGGCGTGGCCCAAATG | 547 |
| 11178 | GCCCGGCGTGGCCCAAATGC | 548 |
| 11179 | CCCGGCGTGGCCCAAATGCC | 549 |
| 11180 | CCGGCGTGGCCCAAATGCCA | 550 |
| 11181 | CGGCGTGGCCCAAATGCCAG | 551 |
| 11182 | GGCGTGGCCCAAATGCCAGC | 552 |
| 11183 | GCGTGGCCCAAATGCCAGCC | 553 |
| 11184 | CGTGGCCCAAATGCCAGCCA | 554 |
| 11185 | GCGCGCCCCTCGCTGTGACC | 523 |
| 11186 | TGCGCGCCCCTCGCTGTGAC | 522 |
| 11187 | CTGCGCGCCCCTCGCTGTGA | 521 |
| 11188 | ACTGCGCGCCCCTCGCTGTG | 520 |
| 11189 | AACTGCGCGCCCCTCGCTGT | 519 |
| 11190 | AAACTGCGCGCCCCTCGCTG | 518 |
| 11191 | CAAACTGCGCGCCCCTCGCT | 517 |
| 11192 | CCAAACTGCGCGCCCCTCGC | 516 |
| 11193 | CCCAAACTGCGCGCCCCTCG | 515 |
| 11194 | CCCCAAACTGCGCGCCCCTC | 514 |
| 11195 | ACCCCAAACTGCGCGCCCCT | 513 |
| 11196 | GACCCCAAACTGCGCGCCCC | 512 |
| 11197 | TGACCCCAAACTGCGCGCCC | 511 |
| 11198 | GTGACCCCAAACTGCGCGCC | 510 |
| 11199 | TGTGACCCCAAACTGCGCGC | 509 |
| 11200 | GTGTGACCCCAAACTGCGCG | 508 |
| 11201 | TGTGTGACCCCAAACTGCGC | 507 |
| 11202 | CTGTGTGACCCCAAACTGCG | 506 |
| 11203 | CGGGGAGTGGGACTGCGGCGGGGAGCCG | 580 |
| 11204 | TCGGGGAGTGGGACTGCGGC | 579 |
| 11205 | CTCGGGGAGTGGGACTGCGG | 578 |
| 11206 | ACTCGGGGAGTGGGACTGCG | 577 |
| 11207 | AACTCGGGGAGTGGGACTGC | 576 |
| 11208 | ACTCGCCGAAGGCCCCTGGGGAAC | 718 |
| 11209 | CTCGCCGAAGGCCCCTGGGG | 719 |
| 11210 | TCGCCGAAGGCCCCTGGGGA | 720 |
| 11211 | CGCCGAAGGCCCCTGGGGAA | 721 |
| 11212 | GCCGAAGGCCCCTGGGGAAC | 722 |
| 11213 | CCGAAGGCCCCTGGGGAACA | 723 |
| 11214 | CGAAGGCCCCTGGGGAACAT | 724 |
| 11215 | GACTCGCCGAAGGCCCCTGG | 717 |
| 11216 | AGACTCGCCGAAGGCCCCTG | 716 |
| 11217 | AAGACTCGCCGAAGGCCCCT | 715 |
| 11218 | AAAGACTCGCCGAAGGCCCC | 714 |
| 11219 | AAAAGACTCGCCGAAGGCCC | 713 |
| 11220 | AAAAAGACTCGCCGAAGGCC | 712 |
| 11221 | CAAAAAGACTCGCCGAAGGC | 711 |
| 11222 | CGGGCTGCATGCGTGAGCAGG | 840 |
| 11223 | GGGCTGCATGCGTGAGCAGG | 841 |
| 11224 | GGCTGCATGCGTGAGCAGGC | 842 |
| 11225 | GCTGCATGCGTGAGCAGGCT | 843 |
| 11226 | CTGCATGCGTGAGCAGGCTA | 844 |
| 11227 | TGCATGCGTGAGCAGGCTAG | 845 |
| 11228 | GCATGCGTGAGCAGGCTAGC | 846 |
| 11229 | CATGCGTGAGCAGGCTAGCA | 847 |
| 11230 | ATGCGTGAGCAGGCTAGCAG | 848 |
| 11231 | TGCGTGAGCAGGCTAGCAGC | 849 |
| 11232 | GCGTGAGCAGGCTAGCAGCA | 850 |
| 11233 | CGTGAGCAGGCTAGCAGCAG | 851 |
| 11234 | CCGGGCTGCATGCGTGAGCA | 839 |
| 11235 | CCCGGGCTGCATGCGTGAGC | 838 |
| 11236 | GCCCGGGCTGCATGCGTGAG | 837 |
| 11237 | AGCCCGGGCTGCATGCGTGA | 836 |
| 11238 | CAGCCCGGGCTGCATGCGTG | 835 |
| 11239 | GCAGCCCGGGCTGCATGCGT | 834 |
| 11240 | TGCAGCCCGGGCTGCATGCG | 833 |
| 11241 | CTGCAGCCCGGGCTGCATGC | 832 |
| 11242 | TCTGCAGCCCGGGCTGCATG | 831 |
| 11243 | CTCTGCAGCCCGGGCTGCAT | 830 |
| 11244 | CCTCTGCAGCCCGGGCTGCA | 829 |
| 11245 | TCCTCTGCAGCCCGGGCTGC | 828 |
| 11246 | TTCCTCTGCAGCCCGGGCTG | 827 |
| 11247 | CTTCCTCTGCAGCCCGGGCT | 826 |
| 11248 | ACTTCCTCTGCAGCCCGGGC | 825 |
| 11249 | CACTTCCTCTGCAGCCCGGG | 824 |
| 11250 | ACACTTCCTCTGCAGCCCGG | 823 |
| 11251 | CACACTTCCTCTGCAGCCCG | 822 |
| 11252 | CGGCAGGCGGTTTAGGCTGTGGCTG | 885 |
| 11253 | GGCAGGCGGTTTAGGCTGTG | 886 |
| 11254 | GCAGGCGGTTTAGGCTGTGG | 887 |
| 11255 | CAGGCGGTTTAGGCTGTGGC | 888 |
| 11256 | AGGCGGTTTAGGCTGTGGCT | 889 |
| 11257 | GGCGGTTTAGGCTGTGGCTG | 890 |
| 11258 | GCGGTTTAGGCTGTGGCTGA | 891 |
| 11259 | CGGTTTAGGCTGTGGCTGAC | 892 |
| 11260 | ACGGCAGGCGGTTTAGGCTG | 884 |
| 11261 | AACGGCAGGCGGTTTAGGCT | 883 |
| 11262 | GAACGGCAGGCGGTTTAGGC | 882 |
| 11263 | TGAACGGCAGGCGGTTTAGG | 881 |
| 11264 | CTGAACGGCAGGCGGTTTAG | 880 |
| 11265 | GCTGAACGGCAGGCGGTTTA | 879 |
| 11266 | GGCTGAACGGCAGGCGGTTT | 878 |
| 11267 | AGGCTGAACGGCAGGCGGTT | 877 |
| 11268 | CAGGCTGAACGGCAGGCGGT | 876 |
| 11269 | TCAGGCTGAACGGCAGGCGG | 875 |
| 11270 | CTCAGGCTGAACGGCAGGCG | 874 |
| 11271 | TCTCAGGCTGAACGGCAGGC | 873 |
| 11272 | CTCTCAGGCTGAACGGCAGG | 872 |
| 11273 | CCTCTCAGGCTGAACGGCAG | 871 |
| 11274 | GCCTCTCAGGCTGAACGGCA | 870 |
| 11275 | AGCCTCTCAGGCTGAACGGC | 869 |
| 11276 | CAGCCTCTCAGGCTGAACGG | 868 |
| 11277 | GCAGCCTCTCAGGCTGAACG | 867 |
| 11278 | CCGATTCGGATTGCTCCAGCTGG | 962 |
| 11279 | CGATTCGGATTGCTCCAGCT | 963 |
| 11280 | GATTCGGATTGCTCCAGCTG | 964 |
| 11281 | ATTCGGATTGCTCCAGCTGG | 965 |
| 11282 | TTCGGATTGCTCCAGCTGGT | 966 |
| 11283 | TCGGATTGCTCCAGCTGGTA | 967 |
| 11284 | CGGATTGCTCCAGCTGGTAA | 968 |
| 11285 | ACCGATTCGGATTGCTCCAG | 961 |
| 11286 | AACCGATTCGGATTGCTCCA | 960 |
| 11287 | TAACCGATTCGGATTGCTCC | 959 |
| 11288 | TTAACCGATTCGGATTGCTC | 958 |
| 11289 | CGCACCCACTCAGTTGCCACGGG | 1008 |
| 11290 | GCACCCACTCAGTTGCCACG | 1009 |
| 11291 | CACCCACTCAGTTGCCACGG | 1010 |
| 11292 | ACCCACTCAGTTGCCACGGG | 1011 |
| 11293 | CCCACTCAGTTGCCACGGGA | 1012 |
| 11294 | CCACTCAGTTGCCACGGGAC | 1013 |
| 11295 | CACTCAGTTGCCACGGGACA | 1014 |
| 11296 | ACTCAGTTGCCACGGGACAC | 1015 |
| 11297 | CTCAGTTGCCACGGGACACA | 1016 |
| 11298 | TCAGTTGCCACGGGACACAC | 1017 |
| 11299 | CAGTTGCCACGGGACACACC | 1018 |
| 11300 | AGTTGCCACGGGACACACCT | 1019 |
| 11301 | GTTGCCACGGGACACACCTG | 1020 |
| 11302 | TTGCCACGGGACACACCTGC | 1021 |
| 11303 | TGCCACGGGACACACCTGCT | 1022 |
| 11304 | GCCACGGGACACACCTGCTT | 1023 |
| 11305 | CCACGGGACACACCTGCTTT | 1024 |
| 11306 | CACGGGACACACCTGCTTTT | 1025 |
| 11307 | ACGGGACACACCTGCTTTTA | 1026 |
| 11308 | CGGGACACACCTGCTTTTAG | 1027 |
| 11309 | ACGCACCCACTCAGTTGCCA | 1007 |
| 11310 | CACGCACCCACTCAGTTGCC | 1006 |
| 11311 | TCACGCACCCACTCAGTTGC | 1005 |
| 11312 | TTCACGCACCCACTCAGTTG | 1004 |
| 11313 | TTTCACGCACCCACTCAGTT | 1003 |
| 11314 | TTTTCACGCACCCACTCAGT | 1002 |
| 11315 | CTTTTCACGCACCCACTCAG | 1001 |
| 11316 | CCTTTTCACGCACCCACTCA | 1000 |
| 11317 | CCCTTTTCACGCACCCACTC | 999 |
| 11318 | CCCCTTTTCACGCACCCACT | 998 |
| 11319 | CCCCCTTTTCACGCACCCAC | 997 |
| 11320 | CCCCCCTTTTCACGCACCCA | 996 |
| 11321 | TCCCCCCTTTTCACGCACCC | 995 |
| 11322 | ATCCCCCCTTTTCACGCACC | 994 |
| 11323 | GATCCCCCCTTTTCACGCAC | 993 |
| 11324 | TGATCCCCCCTTTTCACGCA | 992 |
| 11325 | ATGATCCCCCCTTTTCACGC | 991 |
| 11326 | GATGATCCCCCCTTTTCACG | 990 |
| 11327 | CGGAGACCCACAACGCAACACACC | 1099 |
| 11328 | GGAGACCCACAACGCAACAC | 1100 |
| 11329 | GAGACCCACAACGCAACACA | 1101 |
| 11330 | AGACCCACAACGCAACACAC | 1102 |
| 11331 | GACCCACAACGCAACACACC | 1103 |
| 11332 | ACCCACAACGCAACACACCT | 1104 |
| 11333 | CCCACAACGCAACACACCTG | 1105 |
| 11334 | CCACAACGCAACACACCTGA | 1106 |
| 11335 | CACAACGCAACACACCTGAA | 1107 |
| 11336 | ACAACGCAACACACCTGAAC | 1108 |
| 11337 | CAACGCAACACACCTGAACT | 1109 |
| 11338 | AACGCAACACACCTGAACTG | 1110 |
| 11339 | ACGCAACACACCTGAACTGG | 1111 |
| 11340 | CGCAACACACCTGAACTGGG | 1112 |
| 11341 | CCGGAGACCCACAACGCAAC | 1098 |
| 11342 | GCCGGAGACCCACAACGCAA | 1097 |
| 11343 | TGCCGGAGACCCACAACGCA | 1096 |
| 11344 | GTGCCGGAGACCCACAACGC | 1095 |
| 11345 | TGTGCCGGAGACCCACAACG | 1094 |
| 11346 | ATGTGCCGGAGACCCACAAC | 1093 |
| 11347 | AATGTGCCGGAGACCCACAA | 1092 |
| 11348 | AAATGTGCCGGAGACCCACA | 1091 |
| 11349 | GAAATGTGCCGGAGACCCAC | 1090 |
| 11350 | TGAAATGTGCCGGAGACCCA | 1089 |
| 11351 | CTGAAATGTGCCGGAGACCC | 1088 |
| 11352 | TCTGAAATGTGCCGGAGACC | 1087 |
| 11353 | CTCTGAAATGTGCCGGAGAC | 1086 |
| 11354 | CCTCTGAAATGTGCCGGAGA | 1085 |
| 11355 | GCCTCTGAAATGTGCCGGAG | 1084 |
| 11356 | AGCCTCTGAAATGTGCCGGA | 1083 |
| 11357 | GAGCCTCTGAAATGTGCCGG | 1082 |
| 11358 | TGAGCCTCTGAAATGTGCCG | 1081 |

Hot Zones (Relative upstream location to gene start site)
1-750
800-1200

### Examples

Genetic Code (5′ Upstream Region)

(SEQ ID NO: 11983)

CTTTCCTCCAAGGACTGAAACAGACAAGGATACCCCCTCTTACCACTGTT
ATTCTACATAGTGCAGAAAGTCCTGGCCAGAGCTATCAGGCAAGAGGAAG
AAAGGAAGGGTATCCAAACTGGAAAGGAAGAAGTGGTGAGAAAGTTTTAA
TTTTATTTTTTTGCATGTAGTTATTAAGTTTTCCTAGCACCATTTATTAA
AGAGACTGTTTTTTTCCATTGTATGTTCTTTACAGCTTTGTCACAGATTA
GTTGGTTGTAAGTGCATGGATTTATATGTGGATTCTCTATTCTGCTCCAT
TGGTCTATGTGTCTGTTTCTATGCCAATACTGTGCTGTTTTGGTTACTAT
AGCTTTGTAGTAAATTTTGAAGTTAGGTAGTGTGATGCCTCCAGCTTTGT
TCTTTTTGCTCAGGATTGCTTTGGCTATTCAGGGTCTTTTGTGGTTTCTT
ATAAATTTTAGGAATTTTTCTGTTTCGTGAAGAATGTCATTTGTATTTTG
ATAGGAATTGCATTGAATCTGTAAATTGCTTTAGGTAGTATTGTAATTTT
AACAATATTAGTTCTTCCAGTCTATCACGGAGTATATTTCTGGGTTTTTG
TGTCCTCTTCGATTTCTTTTATCAGAGTTTTATTTATAGTTTTTCCTTGC
ATAGATATTTTACTTTTTTAGTTAAATTGATTCCTAGGTATTGTACATTT
TTGTAGCTATTGTAAAAGGAACTGCTTTCTTGATTTTTATTCAGATTGTT
CACTGTTGTCATATAAATGCTATTGATTTTTGTATGTTGATTTTGCATCC
TGCAACTTTACTGAATCAGATCTAACAGCTTTTAGGTGGACTCTTTAGAT
TTTTCTAGGTATAAGATCATGTAGTCTGCAAACAAACCTAATTTGACTTC
TTCCTTTCCAATTTGGATACCCTTCCTTTCTTCCTCTTGCCTGATAGCTC
TGGCCAGGACTTTCTGTACTACGTAGAATAACAGTGGTGAGAGGGGGTAT
CCTTGTCTGTTTCAGCCCTTGGAGGAAAGGCCTTCATTTTTTCTGTGTTC
AGTATGATGTTGACTGTAGGTTTGTCATATATGGCCTTCATTATTTTGAA
GTATGTTCCTTCTATATCCATTTTGGTGAGAATTTTTATCATAAAGGAAT
GTTGAATTTTATCAAATGCTATCTCAGCATCTATTGAAATAAGTATACCG
TTTCTGTTTTTGATTCTGTTAATGACTTATATTTAGATGGTATTTAAAGA
CATAGGAAATGGGTTAGATCTCCTAAAGAGGGAAGATGGAAAGAGAATGA
AAGAGTTTCCAGAATATAGCCCTGGGGGTTTCCCGCACTTATGGCGGAGG
GAAGGGAGCCATCAGAGGAAACTCAGAGTGGCCAGATAGAAAGGCAGGAA
AAAGCTTAATAAGGTGGTATCATGAGAAGAGAGTCTTCCTAGAAGGAGAA
ATGCTTACTCTTGATAAAAACTGAAAAATAGGGTGAGTCCACATTAGAAT
TCTCGCTACTGGGGAGGACTTTCTACACAAGGTTAGAGATCACTTGTATT
TTGTCTACTATAATTTTAGTGCATAATGCATTTTTTTGGCATATAGTGGG
CACTTATATACATATTAATCTGATGACATTTAATAATTGAGTGCCCAAAT
ATTTATGTCGAGTTGAGAGCTAGGGATAGGCATGAGTCTTTCTTAAACAT
CCTTCTCTTCTCCTTCCTCCACCCCCTCACTTGCCTTCAGAAGCATTGAT
CAGAGAGATAGCAGTATCTTCAGTTTTTTTTAAAGCAACATGAAACACAC
TTTATTCCTGCTAACATTAGGAAAAGCGAGCTGTTTTCCAAGCCCTGGAG
GAAGGAAATTCAGCTAACTAACGTGAGGTAATGTAGGGTGGCTATTTCTT
GAAAGGTAGTGAATCATAACTATAACCATACTATGGAAAAAAGTCCTGCC
TTACCAACCACTCCACTGACTGCTTGTCACCAAAACTACGCTATGAAACG
AATTGTGTTGAGTGGCTTTCATTGTAAAAGATTTTGGTGAAGGGAGGGAA
AGAAACTGGTAGGGGTTCAGATCAGAAGATCTGGCTTTGCCAGTTTCTGG
AGGGTGTCAGAATGGCTTCAACATACCTACTTCCTTGGCCTCAACTGGAG
GTTTTGTAGCTGTAAACAAGAAGGATTGCATAGTTCAGAATAACGACACT
GTAAGCTCATTGTGGAACTGGGTTAAAATCAGCATGTAGATCTACTAAGA
AAGAAACACACTCAGCACTACTACAGAAAGAAACAGCCATGGGCCCTGAT
TGTTAGCTTTCTGGAAGCCATTTCATTTTTACAATAGATTTATCACACAC
TGTATTGACTTTTTCCAGTATAGAGTAAGAGAAAGTTAATATTCCTCATG
TTTTGTCTGTTGACAGACTGAAAATAATTGCATTGAGTTTGGCTAGAATA
TCCTGTCATTCCATAAACATCTCAAACTCCACATGGCTAAAACTTAACCC
ATCCATGCCCCCATCTGCATGCACACATACATGCATATAACTTCATTTCT
CAGTGTTTTTTTCTCCATGAATGGTAGCACCATTCTCTTTCAAGAAAGAG
AAATACTTCCCCTTGGGATTATCCTATCTCTACTTATTGCTGCAGGGGCT
TCCAAAATTAGGTTTTCTGTGTTCAGTCTTGCATTCACACTTCTGAAACC
CAGAGCTGACCGAGACAAATTCTTCAACTTCCTGTCAGTCCCAACATAGA
TTTAAAATTCCTAACCTGGCTCATGAGGTTCACCATGTATTCCTGCTTCC
CTAAGTCAGCCTCATATCACACCAACCTGTGTGTGGAGTGGCTAAATACT
CTAGCCAGGCAAGCTCTCTCAGTTCTTCTACCTGGCTCCTCTGGAGCCCT
CCTTATTGCTCATCCCATTCTTAGCCTGATTCAAGATTCCTGGTCCTTCA
AATCTCTCTTTAAGTGTCCTTACCTGGATCTTTCTCTAGTTAGTACAAAT
TTTTCTATCTACCATTGGAGCGAACATTTTTTGAAACTTTGTATCAGTCC
TGCCTTACTCTTGGTGGAATCCTGTGGTCCTAGTCAAGTGCCTGCTCCAT
GAATGTGCTGAATAAATGAATAAGCATTTTAATTGTGTATCTGTCATTAG
TGTCAGATGTGTTATTTATTCCAGCATGGTTTTAGCACACAGACACACTC
TTTGATGCAGACTTTTCTTTTCTTTTTACATATAGCAACAATAAAAAACT
AGACTTTCATCTCCTGAAAATATCAGTCTAATAATCACCTATGGCTGTCT
CTCTGGTTGCTGAAGGAAAAAAAAAAAAAAGGCAGGGCACACCTGGATTG
CATTAGAATGAGACTCACTACCCAGTTCAGGTGTGTTGCGTTGTGGGTCT
CCGGCACATTTCAGAGGCTCATTAGGACCCTGACCCCACACTGGGGTTTA
CACCCCTAAAAGCAGGTGTGTCCCGTGGCAACTGAGTGGGTGCGTGAAAA
GGGGGGATCATCAATTACCAGCTGGAGCAATCCGAATCGGTTAAAGTGAA
TCAAGTCACAGTGCTTCCTTAACCCAACCTCTCTGTTGGGGTCAGCCACA
GCCTAAACCGCCTGCCGTTCAGCCTGAGAGGCTGCTGCTAGCCTGCTCAC
GCATGCAGCCCGGGCTGCAGAGGAAGTGTGGGGAGGAAGGAAGTGGGTAT
AGAAGGGTGCTGAGATGTGGGTCTTGAAGAGAATAGCCATAACGTCTTTG
TCACTAAAATGTTCCCCAGGGGCCTTCGGCGAGTCTTTTTGTTTGGTTTT
TTGTTTTTAATCTGTGGCTCTTGATAATTTATCTAGTGGTTGCCTACACC
TGAAAAACAAGACACAGTGTTTAACTATCAACGAAAGAACTGGACGGCTC
CCCGCCGCAGTCCCACTCCCCGAGTTTGTGGCTGGCATTTGGGCCACGCC
GGGCTGGGCGGTCACAGCGAGGGGCGCGCAGTTTGGGGTCACACAGCTCC
GCTTCTAGGCCCCAACCACCGTTAAAAGGGGAAGCCCGTGCCCCATCAGG
TCCGCTCTTGCTGAGCCCAGAGCCATCCCGCGCTCTGCGGGCTGGGAGGC
CCGGGCCAGGACGCGAGTCCTGCGCAGCCGAGGTTCCCCAGCGCCCCCTG
CAGCCGCGCGTAGGCAGAGACGGAGCCCGGCCCTGCGCCTCCGCACCACG
CCCGGGACCCCACCCAGCGGCCCGTACCCGGAGAAGCAGCGCGAGCACCC
GAAGCTCCCGGCTGGCGGCAGAAACCGGGAGTGGGGCCGGGCGAGTGCGC
GGCATCCCAGGCCGGCCCGAACGCTCCGCCCGCGGTGGGCCGACTTCCCC
TCCTCTTCCCTCTCTCCTTCCTTTAGCCCGCTGGCGCCGGACACGCTGCG
CCTCATCTCTTGGGGCGTTCTTCCCCGTTGGCCAACCGTCGCATCCCGTG
CAACTTTGGGGTAGTGGCCGTTTAGTGTTGAATGTTCCCCACCGAGAGCG
CATG

TTR

Transthyretin is a 55 kDa protein that exists as a quaternary structure consisting of four monomers binding as two homodimers to create two thyroxine binding sites per tetramer. The dimer-dimer interface comes apart during the process of tetramer dissociation. TTR misfolding and aggregation is known to be associated with amyloid diseases such as senile systemic amyloidosis, familial amyloid polyneuropathy (FAP) and familial amyloid cardiomyopathy (Foss et al. 2005 Biochemistry 44 (47): 15525-33; Zeldenrust SR and Benson Md. (2010). Protein misfolding diseases: current and emerging principles and therapies. New York: Wiley. Westermark et al., Proc. Natl. Acad. Sci. U.S.A. 87 (7): 2843-5. TTR is predominantly synthesized in the liver and choroid plexus for secretion into blood and CNS, respectively. FAP is characterized by pain, paresthesia, muscular weakness, autonomic dysfunction due to the systemic deposition of variants of the transthyretin protein. A common mutations include the replacement of valine by methionine at position 30 (TTR V30M) or valine by isoleucine (TTR V122L). The misfolding of dissociated monomers is believed to cause aggregation into a variety of structures including amyloid fibrils. Treatment of familial TTR amyloid disease has historically relied on liver transplantation as a crude form of gene therapy. Recent approaches include molecules to kinetically stabilize the TTR tetramer or blocking the synthesis of TTR monomers by siRNA and antisense therapeutics.

Protein: TTR Gene: TTR (Homo sapiens, chromosome 18, 29171730-29178987 [NCBI Reference Sequence NC_{—}000018.9]; start site location: 29171866; strand: positive)

Gene Identification

| | |
|---|---|
| GeneID | 7276 |
| HGNC | 12405 |
| HPRD | 01447 |
| MIM | 176300 |

Targeted Sequences

| | | | Relative upstream |
|---|---|---|---|
| Sequence | Design | | location to gene start |
| ID No: | ID | Sequence (5′-3′) | site |
| 11359 | | CAACGCCCTGGCTCGAGTGCAGTGGCACG | 775 |
| 11432 | | CTACTATCTCAGATACTCGGCCAACTCG | 1749 |
| 11450 | | CACGCGTTTCAGCACTGCACCCTGTTG | 2086 |

Target Shift Sequences

| | | Relative |
|---|---|---|
| | | upstream |
| Sequence | | location |
| ID | | to gene |
| No: | Sequence (5′-3′) | start site |
| 11359 | CAACGCCCTGGCTCGAGTGCAGTGGCACG | 775 |
| 11360 | AACGCCCTGGCTCGAGTGCA | 776 |
| 11361 | ACGCCCTGGCTCGAGTGCAG | 777 |
| 11362 | CGCCCTGGCTCGAGTGCAGT | 778 |
| 11363 | GCCCTGGCTCGAGTGCAGTG | 779 |
| 11364 | CCCTGGCTCGAGTGCAGTGG | 780 |
| 11365 | CCTGGCTCGAGTGCAGTGGC | 781 |
| 11366 | CTGGCTCGAGTGCAGTGGCA | 782 |
| 11367 | TGGCTCGAGTGCAGTGGCAC | 783 |
| 11368 | GGCTCGAGTGCAGTGGCACG | 784 |
| 11369 | GCTCGAGTGCAGTGGCACGA | 785 |
| 11370 | CTCGAGTGCAGTGGCACGAT | 786 |
| 11371 | TCGAGTGCAGTGGCACGATC | 787 |
| 11372 | CGAGTGCAGTGGCACGATCA | 788 |
| 11373 | GAGTGCAGTGGCACGATCAC | 789 |
| 11374 | AGTGCAGTGGCACGATCACA | 790 |
| 11375 | GTGCAGTGGCACGATCACAG | 791 |
| 11376 | TGCAGTGGCACGATCACAGC | 792 |
| 11377 | GCAGTGGCACGATCACAGCT | 793 |
| 11378 | CAGTGGCACGATCACAGCTC | 794 |
| 11379 | AGTGGCACGATCACAGCTCG | 795 |
| 11380 | GTGGCACGATCACAGCTCGC | 796 |
| 11381 | TGGCACGATCACAGCTCGCT | 797 |
| 11382 | GGCACGATCACAGCTCGCTG | 798 |
| 11383 | GCACGATCACAGCTCGCTGC | 799 |
| 11384 | CACGATCACAGCTCGCTGCA | 800 |
| 11385 | ACGATCACAGCTCGCTGCAG | 801 |
| 11386 | CGATCACAGCTCGCTGCAGC | 802 |
| 11387 | GATCACAGCTCGCTGCAGCC | 803 |
| 11388 | ATCACAGCTCGCTGCAGCCT | 804 |
| 11389 | TCACAGCTCGCTGCAGCCTT | 805 |
| 11390 | CACAGCTCGCTGCAGCCTTG | 806 |
| 11391 | ACAGCTCGCTGCAGCCTTGA | 807 |
| 11392 | CAGCTCGCTGCAGCCTTGAC | 808 |
| 11393 | AGCTCGCTGCAGCCTTGACC | 809 |
| 11394 | GCTCGCTGCAGCCTTGACCT | 810 |
| 11395 | CTCGCTGCAGCCTTGACCTC | 811 |
| 11396 | TCGCTGCAGCCTTGACCTCC | 812 |
| 11397 | CGCTGCAGCCTTGACCTCCC | 813 |
| 11398 | GCTGCAGCCTTGACCTCCCG | 814 |
| 11399 | CTGCAGCCTTGACCTCCCGG | 815 |
| 11400 | TGCAGCCTTGACCTCCCGGG | 816 |
| 11401 | GCAGCCTTGACCTCCCGGGC | 817 |
| 11402 | CAGCCTTGACCTCCCGGGCT | 818 |
| 11403 | AGCCTTGACCTCCCGGGCTC | 819 |
| 11404 | GCCTTGACCTCCCGGGCTCA | 820 |
| 11405 | CCTTGACCTCCCGGGCTCAG | 821 |
| 11406 | CTTGACCTCCCGGGCTCAGG | 822 |
| 11407 | TTGACCTCCCGGGCTCAGGT | 823 |
| 11408 | TGACCTCCCGGGCTCAGGTC | 824 |
| 11409 | GACCTCCCGGGCTCAGGTCA | 825 |
| 11410 | ACCTCCCGGGCTCAGGTCAT | 826 |
| 11411 | CCTCCCGGGCTCAGGTCATC | 827 |
| 11412 | CTCCCGGGCTCAGGTCATCC | 828 |
| 11413 | TCCCGGGCTCAGGTCATCCT | 829 |
| 11414 | CCCGGGCTCAGGTCATCCTC | 830 |
| 11415 | CCGGGCTCAGGTCATCCTCC | 831 |
| 11416 | CGGGCTCAGGTCATCCTCCC | 832 |
| 11417 | CCAACGCCCTGGCTCGAGTG | 774 |
| 11418 | TCCAACGCCCTGGCTCGAGT | 773 |
| 11419 | CTCCAACGCCCTGGCTCGAG | 772 |
| 11420 | ACTCCAACGCCCTGGCTCGA | 771 |
| 11421 | CACTCCAACGCCCTGGCTCG | 770 |
| 11422 | TCACTCCAACGCCCTGGCTC | 769 |
| 11423 | CTCACTCCAACGCCCTGGCT | 768 |
| 11424 | TCTCACTCCAACGCCCTGGC | 767 |
| 11425 | GTCTCACTCCAACGCCCTGG | 766 |
| 11426 | GGTCTCACTCCAACGCCCTG | 765 |
| 11427 | GGGTCTCACTCCAACGCCCT | 764 |
| 11428 | AGGGTCTCACTCCAACGCCC | 763 |
| 11429 | CAGGGTCTCACTCCAACGCC | 762 |
| 11430 | ACAGGGTCTCACTCCAACGC | 761 |
| 11431 | GACAGGGTCTCACTCCAACG | 760 |
| 11432 | CTACTATCTCAGATACTCGGCCAACTCG | 1749 |
| 11433 | TACTATCTCAGATACTCGGC | 1750 |
| 11434 | ACTATCTCAGATACTCGGCC | 1751 |
| 11435 | CTATCTCAGATACTCGGCCA | 1752 |
| 11436 | TATCTCAGATACTCGGCCAA | 1753 |
| 11437 | ATCTCAGATACTCGGCCAAC | 1754 |
| 11438 | TCTCAGATACTCGGCCAACT | 1755 |
| 11439 | CTCAGATACTCGGCCAACTC | 1756 |
| 11440 | TCAGATACTCGGCCAACTCG | 1757 |
| 11441 | CAGATACTCGGCCAACTCGT | 1758 |
| 11442 | AGATACTCGGCCAACTCGTT | 1759 |
| 11443 | GATACTCGGCCAACTCGTTT | 1760 |
| 11444 | ATACTCGGCCAACTCGTTTG | 1761 |
| 11445 | TACTCGGCCAACTCGTTTGT | 1762 |
| 11446 | ACTCGGCCAACTCGTTTGTA | 1763 |
| 11447 | CTCGGCCAACTCGTTTGTAA | 1764 |
| 11448 | TCGGCCAACTCGTTTGTAAA | 1765 |
| 11449 | CGGCCAACTCGTTTGTAAAA | 1766 |
| 11450 | CACGCGTTTCAGCACTGCACCCTGTTG | 2086 |
| 11451 | ACGCGTTTCAGCACTGCACC | 2087 |
| 11452 | CGCGTTTCAGCACTGCACCC | 2088 |
| 11453 | GCGTTTCAGCACTGCACCCT | 2089 |
| 11454 | CGTTTCAGCACTGCACCCTG | 2090 |
| 11455 | GCACGCGTTTCAGCACTGCA | 2085 |
| 11456 | TGCACGCGTTTCAGCACTGC | 2084 |
| 11457 | GTGCACGCGTTTCAGCACTG | 2083 |
| 11458 | TGTGCACGCGTTTCAGCACT | 2082 |
| 11459 | CTGTGCACGCGTTTCAGCAC | 2081 |
| 11460 | ACTGTGCACGCGTTTCAGCA | 2080 |
| 11461 | TACTGTGCACGCGTTTCAGC | 2079 |
| 11462 | CTACTGTGCACGCGTTTCAG | 2078 |
| 11463 | TCTACTGTGCACGCGTTTCA | 2077 |
| 11464 | ATCTACTGTGCACGCGTTTC | 2076 |
| 11465 | AATCTACTGTGCACGCGTTT | 2075 |
| 11466 | AAATCTACTGTGCACGCGTT | 2074 |
| 11467 | AAAATCTACTGTGCACGCGT | 2073 |
| 11468 | CAAAATCTACTGTGCACGCG | 2072 |
| 11469 | GCAAAATCTACTGTGCACGC | 2071 |
| 11470 | AGCAAAATCTACTGTGCACG | 2070 |

Hot Zones (Relative upstream location to gene start site)
735-915
1185-1275
1725-1815
2085-2175

### Examples

Genetic Code (5′ Upstream Region)

(SEQ ID NO: 11984)

AACTGGGCAGGCCTCAGGAAACTTACAATCATGGTAGAAGGTGAAGGGGA
AGCAAAGCACCTTCCTCACAAGGCGTCAGGAAGAAGTGCCAAGCAAAGGG
GGAAAAGCCCCTTGTAAAACTACCAGAACCTGTGAGAACTCAATCACTAT
CACAAGAACAGCATGAGGGAACCGCCCCTCGTGATTCAATTACCTCCACC
TGGTCTCTCCCTTGACACATGGGGATTATGGGTGTTACAATTCAAGATGA
GATTTGGGTGGGGACACAAAGCCTAACCATATCAAGGATCAAGTGGTGGG
TTGAAACTAACAGGATGAGATATATCAGATACAAACACAGGGTCCCATAT
TTGGGTTAAAATTCATAAATGATCAAAGCACAGGATGACAGATAATATAG
GTCATTTTAGATTATTGTGGCCAACAGATCACAGTGGGTAGTGTTATGAC
GAAGGGAGGGTCACAGTTACTACAGTTACAGATGGATTCTGGGTACAACA
TTTGCACTAAAGTGCCTTTGCCAAGGGAGGCAACAGTCTCGACATCCTGT
GGCCTGATCTACTTCAGGGACTGTGTCTTGTTCAGAGCATCACATTTGAA
GAGAACTTTGACCAAGGGGAATATGCCAGAAAAGGAAGTTCGGGATGCTG
AGGATCTTAGGAACTATGTCTAAACAAGATTCATTCACAGAAGTGGGAAT
GTCTATTTGGCAAAAAGAAAATACTACTTACATGGCTGTTGGAAGACCAG
CAATCACAAACTCAGTTTTTCAAAAGGCTGGGCAGAAACACAGATGAAAG
AAACAGGCCATGTTTAAGAAAAGATAAAAGCTCACGCATGATATGCCACT
AGAGAATCACCTAGCCTCAGTGTTGGCGGGGAGGCCTGGGGAGTCTTGAT
GTCTGAGAGTGACATTCTGATGATCACTGTCATGTGTAAATGTTGGCCTA
AAGCTGCCAATATTTTTGATTTAAGAGAAGCAAGAAATGCAAATTTTTAT
GCAGCATGTCTCAATTTTTAATTTTGGCAACTATTACAAAATGTTTAAAG
AGACTCTGTGCAGCCCAAATATAACATATCTATGGGCTGATGGCAGCCCA
GCGTTGCCAGTTCACAGGGTCTACAAGAGATGATTCTTAGTTTCAACAGG
GTGCAGTGCTGAAACGCGTGCACAGTAGATTTTGCTTCGGTTATGAAAGA
ACTTCCAAATATTTATGATTCATAGCCAGAGAAAAGGCTCTCTATCCAGG
TTCTGAACAATAGGAAATCATCAAGAGGATATTGGATGACAATATATGAA
AGATGTTATTTGAGAAAGGATTCTCTCCTGAGGCATAGATGTTGAACCAA
ATTCTATTAGTTATGCTTTTACAGCAAGATAGTGGTTTACAGCTTACAAA
AGGCTTGTACATCCTCTCATATTAAAAGTTATTAGAACAGTCCTTTGAAG
TAGAAAAGTAGGCATTTCTATTTTACAAACGAGTTGGCCGAGTATCTGAG
ATAGTAGATAACTCATAGAAGGTCATCCGGGAAACGGGGCAGCAGAACTG
GGATCGAATGACTCTGGTCATCCAACTCCAAATGCAAAAGTCTTTCTGCT
GCTGCTTCCTAGTTAAACTCTAAGGGTCTAAGACTCCATTCCTAGTTATG
GTCTCAACTACATTTGCTCATTGCTGTGAGGGGTCAACCCACCTCCCGGA
GTCCTCTCCTGCACATTCTCATGTTCCTGAAAGGCTTTTCTGTCCCTTCC
ACTACTCCCTGTAAGCTCCTGTGCTTCACAATTTCTTGTTGAATTTTTTC
TAATCTGACTCTATCAGTTATGGGAATGTTCCCTCAATTCTTAGTGCTCC
AAACCGGACTTGCTCTTGGCTTGTATTTGTCCAAAATATTTGTCTTCTCT
ATGTTTTCTACATGTTTGTCTTATAAGGACAAAAACCTGCCTTAGTTTAT
CCATGAACAAAGCCACGCATGCTAGTGGACACACACACACATGCGCGTGC
GCGCGCACACACACACACACACACATACACACAGAGACTTTGTATGTGAG
TAATGAATCATCAAATCATCATAATTTCTGGACTTGTATTAATAAGTCGG
CCAGGAGGAAAAGAATCTGCTGTCAATCATGGCTTCTGGTTCTCACAGTC
ATCTCTACTTTCTTCCAGCAAGTTTGGTTCTGTCAAAAACCAGCTGTCAG
CCTTGTTCCTGCATGCCCAATGCAGAAGAGTCAGTAAAGAAGATTTGGTT
CTCTGTATTTCAGGGGCATCAATGCCAGGTTGAAATATGCCATTCTGGCC
CAGCTCAGTGGCTCACACGTGTAATCCCAGCACTTTGGAAGGCCAAAGCG
GGTGGATTGCTTGAGCTCAGGAGTTCGAGACCAGCCTGGGCAAGAGGCTG
AGGTGGGAGGATGACCTGAGCCCGGGAGGTCAAGGCTGCAGCGAGCTGTG
ATCGTGCCACTGCACTCGAGCCAGGGCGTTGGAGTGAGACCCTGTCAAAA
AAAAAAAAAAAAAGGAAGGAAAAAAGGAAGGAAGGAAGGGAGGGAGGGAA
GATGCCATTCTTAGATTGAAGTGGACTTTATCTGGGCAGAACACACACAC
ACATACACACATGCACACACACATTGTGGAGAAATTGCTGACTAAGCAAA
GCTTCCAAATGACTTAGTTTGGCTAAAATGTAGGCTTTTAAAAATGTGAG
CACTGCCAAGGGTTTTTCCTTGTTGACCCATGGATCCATCAAGTGCAAAC
ATTTTCTAATGCACTATATTTAAGCCTGTGCAGCTAGATGTCATTCAACA
TGAAATACATTATTACAACTTGCATCTGTCTAAAATCTTGCATCTAAAAT
GAGAGACAAAAAATCTATAAAAATGGAAAACATGCATAGAAATATGTGAG
GGAGGAAAAAATTACCCCCAAGAATGTTAGTGCACGCAGTCACACAGGGA
GAAGACTATTTTTGTTTTGTTTTGATTGTTTTGTTTTGTTTTGGTTGTTT
TGTTTTGGTGACCTAACTGGTCAAATGACCTATTAAGAATATTTCATAGA
ACGAATGTTCCGATGCTCTAATCTCTCTAGACAAGGTTCATATTTGTATG
GGTTACTTATTCTCTCTTTGTTGACTAAGTCAATAATCAGAATCAGCAGG
TTTGCAGTCAGATTGGCAGGGATAAGCAGCCTAGCTCAGGAGAAGTGAGT
ATAAAAGCCCCAGGCTGGGAGCAGCCATCACAGAAGTCCACTCATTCTTG
GCAGGATG

CD68

CD68 (Cluster of Differentiation 68) is a glycoprotein that is expressed on monocytes/macrophages. It is often used as a marker for monocytes, histiocytes, giant cells, Kupffer cells, and osteoclasts. CD68 has been used to distinguish between diseases of similar appearance, e.g. (1) for monocytes of lymphoid origin and (2) macrophages to diagnose conditions related to proliferation or abnormality of these cells, such as malignant histiocytosis, histiocytic lymphoma, and Gaucher's disease. CD68 primarily localizes to lysosomes and endosomes with a smaller fraction circulating to the cell surface. It is a type I integral membrane protein with a heavily glycosylated extracellular domain and binds to tissue- and organ-specific lectins or selectins. The protein is also a member of the scavenger receptor family and has been reported to bind LDL. Scavenger receptors typically function to clear cellular debris, promote phagocytosis, and mediate the recruitment and -activation of macrophages. Alternative splicing of the gene results in multiple transcripts encoding different isoforms of CD68.

Protein: CD68 Gene: CD68 (Homo sapiens, chromosome 17, 7482805-7485429 [NCBI Reference Sequence: NC_{—}000017.10]; start site location: 7482996; strand: positive)

Gene Identification

| | |
|---|---|
| GeneID | 968 |
| HGNC | 1693 |
| MIM | 153634 |

Targeted Sequences

| | | Relative |
|---|---|---|
| | | upstream |
| Sequence | | location to |
| ID | Sequence (5′-3′) | gene start site |
| 11989 | CGAGAACATGGCTTTCCAGCGTCTG | 520 |

Hot Zones (Relative upstream location to gene start site)
1-600

Genetic Code (5′ Upstream Region)

(SEQ ID NO: 11985)

GCCACATTTGCCATATCGATTCTGCAGCAGATTGAATTAGATCTAAAAGC
CACCCAGGCCTTGGTCCTAGCACCCACTCGAGAATTGGCTCAGCAGGTAA
GAGTGGCTTCTATTCCCTCCTTCAGGGCTGATTTAGGGATGATGAGTATA
ATCCAAGGACCAGAGAAGTCTTCTCTGATCACCACCTTGGGAGGAAGACA
TGGGTGCCCTAACACTCTCGAGACCTGCTGGGTTAATTAAAAGCTATTTC
TTACCCAAACGTAACCATTGCTTCCTCCACCCATTTCCTGAGTCAAATGG
GAAAGCTGTTGGGTGAAGCCTGGCTGGCTGGGCAAGTTTGACTGTGTTCT
GAATAAGCACCTTCACTATGGGCTAAGAGATCCCTTGGTGTGGGGGTGAT
CTTACAGTAGTCAGAGCAGATGGACAGTCCTTTTCACCCTTGCTTAATAG
CCAGAGCTGTTTCATGCCTGGGGCACACACAATTCTAATGCTGGACTTTT
TCCTGGGTCATGCTGCAACACTGATGTCAGAGCATGTTTTTAAATGTTCT
GTGGCAGGGGCAGTGATTATTCTGGGTGTGGATAATGTAAGAAGTTACAG
CAGAGCTCCATTCTAAGGCACTTGGCTCTCAGTTTTCTCAGAGTGAACAT
GCCTCGTAGCTTGGGTCCTATGGCAGGAGTGCAATAGGACATGGATATGC
ATCACCTGTTCTATAAAACTGGTTGCTGGCTGGGTGTGGTGGCTCAACTC
GTATAATCCCAACACTTTGGGAGGCCAAGGCAGGCAGATCTCTTGAGATC
AGGAGTTGGAGACCAGCCTGGCCAACATAGTGAAACCCCGCTTCTACTAA
AAATACAAAAATTAGCCAGGCATGGTGGCGTGTGCCTTTTATCCCAGCTA
CTCGGGAAGCTCAGGCAGGAGAATTTAACCCAGGAGGTGGAGGTTGCAGT
GAGCTGAGATTGTGCCATTGCACTCCAGCCTGGGCAACGAGCAAAGCTCT
GTCTCAAAAAAAGAAAAAAAAAATGGTTGCTGCGTGATGAGGCAGTTGGT
CAAATTAGTTTTCAGAAGGTTAAGGGTTCTAAATATCTAGAGTAAAGAAA
CTGAATTAATTATCTGAGCGGCCTCATTGTGAATCACTGTACACTCAGGA
ACCAGACTGAGTTGAAATCCTGTCTTTGCCACCTATTGACAGCACGATCT
TAAGTGGATTTTAGCCTCTGCCTGTTTCTCAGCTGAATGTGAGTTTAATA
ATAGTGCATGCCCCAAAGTTGTTGGTTAGGAATCAATACATGAAAAACAT
TTAAGAATGGTGCCGGGCACAGTGGTAACTGACATATGAGCACCTGCCTC
TCTCTGCTCAGATACAGAAGGTGGTCATGGCACTAGGAGACTACATGGGC
GCCTCCTGTCACGCCTGTATCGGGGGCACCAACGTGCGTGCTGAGGTGCA
GAAACTGCAGATGGAAGCTCCCCACATCATCGTGGGTACCCCTGGCCGTG
TGTTTGATATGCTTAACCGGAGATACCTGTGTGAGTAATTCGGTTCTCCA
ATCCCCTGGGTCACTTTGCTCTTGTGCACGCTTTCCAGTCTTTCAGCGTA
AGCCAGAGTCATTCCCAAGGATGCTGGTTTCTCTCTGGGGGAAGAGCTGC
TCTGTGATGGAGCCCATGCGTGTCATCTGAGCCTCTGGCTTCCCTGCCAG
TGCAGCCCTGGCAGTGTCCTACTTCCCAGGGCTGTTGTCTGCCTGGCGGG
AAGGTCCTGGGCAAAGGATCAGTCTTTGTACTCTGAGAGCAGACTACTTG
GCTCCTCTCTGTTTTTTATCAGCGAAGTTGGATATATCTCTCCCACATTT
CCCTAATCATATGCTATATATTGGCTTTTTTTTTCTTCTCTAGCCCCCAA
ATACATCAAGATGTTTGTACTGGATGAAGCTGACGAAATGTTAAGCCGTG
GATTCAAGGACCAGATCTATGACATATTCCAAAAGCTCAACAGCAACACC
CAGGTGAGGGCAGTCTTGCTTGAATAGCTAATGATTCTTGAAAAATAGTA
AGTGCCAGGGGAACCATATACTGGATTCTTGAGCCTTTTTATGCATCTGC
TTCAGTTTTAGGTGTGGCTAGGGAAGGGAGCAGGCCTCAGGAAGGAACCA
GCACTCTAAGACTGGCCTTTTTTTCCACTAGGTAGTTTTGCTGTCAGCCA
CAATGCCTTCTGATGTGCTTGAGGTGACCAAGAAGTTCATGAGGGACCCC
ATTCGGATTCTTGTCAAGAAGGAAGAGTTGACCCTGGAGGGTATCCGCCA
GTTCTACATCAACGTGGAACGAGAGGTGGGGCCCAGTGCAGGAGGCGGGC
CTGGTAGTGAGTTGTTGGGTATAGCCCCTGACTGATTTTTGTCCCCCAAC
CTCCAGGAGTGGAAGCTGGACACACTATGTGACTTGTATGAAACCCTGAC
CATCACCCAGGCAGTCATCTTCATCAACACCCGGAGGAAGGTGGACTGGC
TCACCGAGAAGATGCATGCTCGAGATTTCACTGTATCCGCCATGGTGTGT
TTGCCCGCTGCCAGCCTGTTGTGGGTCTGCCCGTCAGAAGTGTCCTACTT
GAAGCCAGGGTTCCTGGAACCCAGGTGCCTACCTGGTCTGCTGCATATTT
GTTTTCTCTTCCAGCATGGAGATATGGACCAAAAGGAACGAGACGTGATT
ATGAGGGAGTTTCGTTCTGGCTCTAGCAGAGTTTTGATTACCACTGACCT
GCTGGTGAGTAGAGGGAACTGATAGCAAAGGCAGAAGGGAGGATCCAAGG
TGATTCCCTCTCCAAGGGGACATCAGTGCCTCTCAGGAAAGTAGCAGCTT
GGAATAGAATCTGGCATGCCTAAGGCCTTTGGGGAACTGGGATGCTTATT
TCCTCTGCCTTCCTTGGCTGCCCACATGGATGCCTAAGTGTCTTCCCTCC
GGGATAGAGTGTCCTCCGTGCACATGCTGAAGAGTTGTCTTTCTTGACGT
AGGCCAGAGGCATTGATGTGCAGCAGGTTTCTTTAGTCATCAACTATGAC
CTTCCCACCAACAGGGAAAACTATATCCACAGGTAAGCGTAGATCTGGAA
CACTCCCCTACCCCTTCACACCTGGCCCTCCCTGGGCTTAAAGCTCCTGA
TATTCCTCATCCCCTTCCTTGTTTTCCAGAATCGGTCGAGGTGGACGGTT
TGGCCGTAAAGGTGTGGCTATTAACATGGTGACAGAAGAAGACAAGAGGA
CTCTTCGAGACATTGAGACCTTCTACAACACCTCCATTGAGGAAATGCCC
CTCAATGTTGCTGACCTCATCTGAGGGGCTGTCCTGCCACCCAGCCCCAG
CCAGGGCTCAATCTCTGGGGGCTGAGGAGCAGCAGGAGGGGGGAGGGAAG
GGAGCCAAGGGATGGACATCTTGTCATTTTTTTTCTTTGAATAAATGTCA
CTTTTTGAGGCAAAAGAAGGAACCGTGAACATTTTAGACACCCTTTTCTT
TGGGGTAGGCTCTTGCCCCAGGCGCCGGCTCTTCTCCCAAAAAAAAAAAA
AAAACACTAATCCATTTCCCTAACCTAGTAACCTCCAGATCCCAGAGGCT
CTCCTCACCTCAGCTGAGCTCCTTTGAAAGTGATTCAAGGGACTATGTCA
CTCAGCCTCATTTGCTGGACCAAATCTGGAGGGAGAACCCCTAAAACCCC
TAAGTGAGGTTGCCCAGGGGGTTGTCCCCAGGTGGGGGGAAGCAGGGGAG
AGAAAATGGTAGCCATTTTTACATTGTTTTGTATAGTATTTATTGATTCA
GGAAACAAACACAAAATTCTGAATAAAATGACTTGGAAACTGCCTGTTTG
GGCTTCTCATTTCTTACCTCCCCTTCCCTCTCCCACCTGCTACTGGGTGC
ATCTCTGCTCCCCCCTTCCCCAGCAGATGGTTACCTTTGGGCTGTTGCTT
TCTTGTCACCATCTGAGTTCTCAGACGCTGGAAAGCCATGTTCTCGGCTC
TGTGAATGACAATGCTGACTGGAGTGCTGCCCCTCTGTAAAGGGCTGGGT
GTGGATGGTCACAAGCCCCTCACATGCCTCAGCCAAGAGGAAGTAGTACA
GGGGTCAGCCCAGAGGTCCAGGGGAAAGGAGTGGAAACCGATTTCCCCAC
CAAGGGAGGGGCCTGTACCTCAGCTGTTCCCATAGCTACTTGCCACAACT
GCCAAGCAAGTTTCGCTGAGTTTGACACATGGATCCCTGTGGATCAACTG
CCCTAGGACTCCGTTTGCACCCATGTGACACTGTTGACTTTGCCCTGATG
AAGCAGGGCCAACAGTCCCCTAACTTAATTACAAAAACTAATGACTAAGA
GAGAGGTGGCTAGAGCTGAGGCCCCTGAGTCAGGCTGTGGGTGGGATCAT
CTCCAGTACAGGAAGTGAGACTTTCATTTCCTCCTTTCCAAGAGAGGGCT
GAGGGAGCAGGGTTGAGCAACTGGTGCAGACAGCCTAGCTGGACTTTGGG
TGAGGCGGTTCAGCCATG

ALK

Anaplastic lymphoma kinase (ALK) also known as ALK tyrosine kinase receptor or CD246 (cluster of differentiation 246) is an enzyme encoded by the ALK gene. ALK is believed to have a putative transmembrane domain and an extracellular domain. ALK is believed to have oncogenic properties in through several ways: mutations, amplified copies, or fusion products with other genes. The t(2; 5) chromosomal translocation is associated with approximately 60% anaplastic large-cell lymphomas (ALCLs) and creates a fusion gene consisting of the ALK gene and the nucleophosmin (NPM) gene: the 3′ half of ALK, derived from chromosome 2 and coding for the catalytic domain, is fused to the 5′ portion of NPM from chromosome 5. The product of NPM-ALK or EML4-ALK fusion genes are oncogenic in lymphoma and non-small cell lung cancers, respectively. In a smaller fraction of ALCL patients, the 3′ half of ALK is fused to the 5′ sequence of TPM3 gene, encoding for tropomyosin 3. In rare cases, ALK is fused to other 5′ fusion partners, such as TFG, ATIC, CLTC1, TPM4, MSN, ALO17, MYH9.

Protein: ALK Gene: ALK (Homo sapiens, chromosome 2, 29415640-30144477 [NCBI Reference Sequence: NC_{—}000002.11]; start site location: 30143525; strand: negative)

Gene Identification

| | |
|---|---|
| GeneID | 238 |
| HGNC | 427 |
| MIM | 105590 |

Targeted Sequences

| | | Relative |
|---|---|---|
| | | upstream |
| Sequence | | location to |
| ID No: | Sequence (5′-3′) | gene start site |
| 11471 | CGCCGGAGGAGGCCGTTTACACTGC | 3 |
| 11530 | CGTGCGCGCAAGTCTCTTGCTTTCC | 132 |
| 11555 | CGCTCTCCGCGCCGAGTGCCGCGCC | 269 |
| 11621 | CGCCTTTTGCGTTCCTTTTGGCTCC | 482 |
| 11681 | CGCAGGCACTGGAGCGGCCCCGGCG | 701 |
| 11794 | CGACCCTCCGAACAGAGGCGGCGGG | 851 |
| 11825 | CGCGCTGCTGCCCGACCCACGCAGT | 1022 |
| 11901 | CGGGTCCGACTTCGGAAAAACAGGT | 1313 |
| 11923 | CGGCCTGTCGGGTAGCACAGGAGTT | 2022 |

Targeted Shift Sequences

| | | Relative |
|---|---|---|
| | | upstream |
| Sequence | | location to |
| ID No: | Sequence (5′-3′) | gene start site |
| 11471 | CGCCGGAGGAGGCCGTTTACACTGC | 3 |
| 11472 | GCCGGAGGAGGCCGTTTACA | 4 |
| 11473 | CCGGAGGAGGCCGTTTACAC | 5 |
| 11474 | CGGAGGAGGCCGTTTACACT | 6 |
| 11475 | GGAGGAGGCCGTTTACACTG | 7 |
| 11476 | GAGGAGGCCGTTTACACTGC | 8 |
| 11477 | AGGAGGCCGTTTACACTGCT | 9 |
| 11478 | GGAGGCCGTTTACACTGCTC | 10 |
| 11479 | GAGGCCGTTTACACTGCTCT | 11 |
| 11480 | AGGCCGTTTACACTGCTCTC | 12 |
| 11481 | GGCCGTTTACACTGCTCTCC | 13 |
| 11482 | GCCGTTTACACTGCTCTCCG | 14 |
| 11483 | CCGTTTACACTGCTCTCCGG | 15 |
| 11484 | CGTTTACACTGCTCTCCGGG | 16 |
| 11485 | GTTTACACTGCTCTCCGGGC | 17 |
| 11486 | TTTACACTGCTCTCCGGGCC | 18 |
| 11487 | TTACACTGCTCTCCGGGCCC | 19 |
| 11488 | TACACTGCTCTCCGGGCCCA | 20 |
| 11489 | ACACTGCTCTCCGGGCCCAG | 21 |
| 11490 | CACTGCTCTCCGGGCCCAGC | 22 |
| 11491 | ACTGCTCTCCGGGCCCAGCC | 23 |
| 11492 | CTGCTCTCCGGGCCCAGCCT | 24 |
| 11493 | TGCTCTCCGGGCCCAGCCTC | 25 |
| 11494 | GCTCTCCGGGCCCAGCCTCA | 26 |
| 11495 | CTCTCCGGGCCCAGCCTCAC | 27 |
| 11496 | TCTCCGGGCCCAGCCTCACC | 28 |
| 11497 | CTCCGGGCCCAGCCTCACCC | 29 |
| 11498 | TCCGGGCCCAGCCTCACCCT | 30 |
| 11499 | CCGGGCCCAGCCTCACCCTT | 31 |
| 11500 | CGGGCCCAGCCTCACCCTTC | 32 |
| 11501 | GGGCCCAGCCTCACCCTTCG | 33 |
| 11502 | GGCCCAGCCTCACCCTTCGC | 34 |
| 11503 | GCCCAGCCTCACCCTTCGCT | 35 |
| 11504 | CCCAGCCTCACCCTTCGCTC | 36 |
| 11505 | CCAGCCTCACCCTTCGCTCT | 37 |
| 11506 | CAGCCTCACCCTTCGCTCTC | 38 |
| 11507 | AGCCTCACCCTTCGCTCTCC | 39 |
| 11508 | GCCTCACCCTTCGCTCTCCC | 40 |
| 11509 | CCTCACCCTTCGCTCTCCCC | 41 |
| 11510 | CTCACCCTTCGCTCTCCCCG | 42 |
| 11511 | TCACCCTTCGCTCTCCCCGA | 43 |
| 11512 | CACCCTTCGCTCTCCCCGAG | 44 |
| 11513 | ACCCTTCGCTCTCCCCGAGA | 45 |
| 11514 | CCCTTCGCTCTCCCCGAGAT | 46 |
| 11515 | CCTTCGCTCTCCCCGAGATG | 47 |
| 11516 | CTTCGCTCTCCCCGAGATGG | 48 |
| 11517 | TTCGCTCTCCCCGAGATGGG | 49 |
| 11518 | TCGCTCTCCCCGAGATGGGA | 50 |
| 11519 | CGCTCTCCCCGAGATGGGAA | 51 |
| 11520 | GCTCTCCCCGAGATGGGAAG | 52 |
| 11521 | CTCTCCCCGAGATGGGAAGA | 53 |
| 11522 | TCTCCCCGAGATGGGAAGAG | 54 |
| 11523 | CTCCCCGAGATGGGAAGAGG | 55 |
| 11524 | TCCCCGAGATGGGAAGAGGC | 56 |
| 11525 | CCCCGAGATGGGAAGAGGCT | 57 |
| 11526 | CCCGAGATGGGAAGAGGCTC | 58 |
| 11527 | CCGAGATGGGAAGAGGCTCT | 59 |
| 11528 | CCGCCGGAGGAGGCCGTTTA | 2 |
| 11529 | CCCGCCGGAGGAGGCCGTTT | 1 |
| 11530 | CGTGCGCGCAAGTCTCTTGCTTTCC | 132 |
| 11531 | GTGCGCGCAAGTCTCTTGCT | 133 |
| 11532 | TGCGCGCAAGTCTCTTGCTT | 134 |
| 11533 | GCGCGCAAGTCTCTTGCTTT | 135 |
| 11534 | CGCGCAAGTCTCTTGCTTTC | 136 |
| 11535 | GCGCAAGTCTCTTGCTTTCC | 137 |
| 11536 | CGCAAGTCTCTTGCTTTCCC | 138 |
| 11537 | GCGTGCGCGCAAGTCTCTTG | 131 |
| 11538 | TGCGTGCGCGCAAGTCTCTT | 130 |
| 11539 | GTGCGTGCGCGCAAGTCTCT | 129 |
| 11540 | TGTGCGTGCGCGCAAGTCTC | 128 |
| 11541 | CTGTGCGTGCGCGCAAGTCT | 127 |
| 11542 | ACTGTGCGTGCGCGCAAGTC | 126 |
| 11543 | GACTGTGCGTGCGCGCAAGT | 125 |
| 11544 | GGACTGTGCGTGCGCGCAAG | 124 |
| 11545 | AGGACTGTGCGTGCGCGCAA | 123 |
| 11546 | GAGGACTGTGCGTGCGCGCA | 122 |
| 11547 | AGAGGACTGTGCGTGCGCGC | 121 |
| 11548 | CAGAGGACTGTGCGTGCGCG | 120 |
| 11549 | CCAGAGGACTGTGCGTGCGC | 119 |
| 11550 | TCCAGAGGACTGTGCGTGCG | 118 |
| 11551 | CTCCAGAGGACTGTGCGTGC | 117 |
| 11552 | TCTCCAGAGGACTGTGCGTG | 116 |
| 11553 | ATCTCCAGAGGACTGTGCGT | 115 |
| 11554 | GATCTCCAGAGGACTGTGCG | 114 |
| 11555 | CGCTCTCCGCGCCGAGTGCCGCGCC | 269 |
| 11556 | GCTCTCCGCGCCGAGTGCCG | 270 |
| 11557 | CTCTCCGCGCCGAGTGCCGC | 271 |
| 11558 | TCTCCGCGCCGAGTGCCGCG | 272 |
| 11559 | CTCCGCGCCGAGTGCCGCGC | 273 |
| 11560 | TCCGCGCCGAGTGCCGCGCC | 274 |
| 11561 | CCGCGCCGAGTGCCGCGCCC | 275 |
| 11562 | CGCGCCGAGTGCCGCGCCCC | 276 |
| 11563 | GCGCCGAGTGCCGCGCCCCC | 277 |
| 11564 | CGCCGAGTGCCGCGCCCCCG | 278 |
| 11565 | GCCGAGTGCCGCGCCCCCGT | 279 |
| 11566 | CCGAGTGCCGCGCCCCCGTC | 280 |
| 11567 | CGAGTGCCGCGCCCCCGTCT | 281 |
| 11568 | GAGTGCCGCGCCCCCGTCTG | 282 |
| 11569 | AGTGCCGCGCCCCCGTCTGT | 283 |
| 11570 | GTGCCGCGCCCCCGTCTGTA | 284 |
| 11571 | TGCCGCGCCCCCGTCTGTAG | 285 |
| 11572 | GCCGCGCCCCCGTCTGTAGC | 286 |
| 11573 | CCGCGCCCCCGTCTGTAGCT | 287 |
| 11574 | CGCGCCCCCGTCTGTAGCTC | 288 |
| 11575 | GCGCCCCCGTCTGTAGCTCG | 289 |
| 11576 | CGCCCCCGTCTGTAGCTCGC | 290 |
| 11577 | GCCCCCGTCTGTAGCTCGCT | 291 |
| 11578 | CCCCCGTCTGTAGCTCGCTG | 292 |
| 11579 | CCCCGTCTGTAGCTCGCTGC | 293 |
| 11580 | CCCGTCTGTAGCTCGCTGCG | 294 |
| 11581 | CCGTCTGTAGCTCGCTGCGC | 295 |
| 11582 | CGTCTGTAGCTCGCTGCGCT | 296 |
| 11583 | GTCTGTAGCTCGCTGCGCTC | 297 |
| 11584 | TCTGTAGCTCGCTGCGCTCG | 298 |
| 11585 | CTGTAGCTCGCTGCGCTCGG | 299 |
| 11586 | TGTAGCTCGCTGCGCTCGGT | 300 |
| 11587 | GTAGCTCGCTGCGCTCGGTA | 301 |
| 11588 | TAGCTCGCTGCGCTCGGTAC | 302 |
| 11589 | AGCTCGCTGCGCTCGGTACA | 303 |
| 11590 | GCTCGCTGCGCTCGGTACAG | 304 |
| 11591 | CTCGCTGCGCTCGGTACAGA | 305 |
| 11592 | TCGCTGCGCTCGGTACAGAG | 306 |
| 11593 | CGCTGCGCTCGGTACAGAGG | 307 |
| 11594 | GCTGCGCTCGGTACAGAGGA | 308 |
| 11595 | CTGCGCTCGGTACAGAGGAA | 309 |
| 11596 | TGCGCTCGGTACAGAGGAAC | 310 |
| 11597 | GCGCTCGGTACAGAGGAACT | 311 |
| 11598 | CGCTCGGTACAGAGGAACTA | 312 |
| 11599 | GCTCGGTACAGAGGAACTAC | 313 |
| 11600 | CTCGGTACAGAGGAACTACT | 314 |
| 11601 | TCGGTACAGAGGAACTACTA | 315 |
| 11602 | CGGTACAGAGGAACTACTAT | 316 |
| 11603 | CCGCTCTCCGCGCCGAGTGC | 268 |
| 11604 | CCCGCTCTCCGCGCCGAGTG | 267 |
| 11605 | TCCCGCTCTCCGCGCCGAGT | 266 |
| 11606 | CTCCCGCTCTCCGCGCCGAG | 265 |
| 11607 | CCTCCCGCTCTCCGCGCCGA | 264 |
| 11608 | GCCTCCCGCTCTCCGCGCCG | 263 |
| 11609 | AGCCTCCCGCTCTCCGCGCC | 262 |
| 11610 | GAGCCTCCCGCTCTCCGCGC | 261 |
| 11611 | TGAGCCTCCCGCTCTCCGCG | 260 |
| 11612 | TTGAGCCTCCCGCTCTCCGC | 259 |
| 11613 | CTTGAGCCTCCCGCTCTCCG | 258 |
| 11614 | CCTTGAGCCTCCCGCTCTCC | 257 |
| 11615 | ACCTTGAGCCTCCCGCTCTC | 256 |
| 11616 | GACCTTGAGCCTCCCGCTCT | 255 |
| 11617 | GGACCTTGAGCCTCCCGCTC | 254 |
| 11618 | GGGACCTTGAGCCTCCCGCT | 253 |
| 11619 | TGGGACCTTGAGCCTCCCGC | 252 |
| 11620 | CTGGGACCTTGAGCCTCCCG | 251 |
| 11621 | CGCCTTTTGCGTTCCTTTTGGCTCC | 482 |
| 11622 | GCCTTTTGCGTTCCTTTTGG | 483 |
| 11623 | CCTTTTGCGTTCCTTTTGGC | 484 |
| 11624 | CTTTTGCGTTCCTTTTGGCT | 485 |
| 11625 | TTTTGCGTTCCTTTTGGCTC | 486 |
| 11626 | TTTGCGTTCCTTTTGGCTCC | 487 |
| 11627 | TTGCGTTCCTTTTGGCTCCT | 488 |
| 11628 | TGCGTTCCTTTTGGCTCCTC | 489 |
| 11629 | GCGTTCCTTTTGGCTCCTCC | 490 |
| 11630 | CGTTCCTTTTGGCTCCTCCA | 491 |
| 11631 | CCGCCTTTTGCGTTCCTTTT | 481 |
| 11632 | GCCGCCTTTTGCGTTCCTTT | 480 |
| 11633 | GGCCGCCTTTTGCGTTCCTT | 479 |
| 11634 | TGGCCGCCTTTTGCGTTCCT | 478 |
| 11635 | CTGGCCGCCTTTTGCGTTCC | 477 |
| 11636 | CCTGGCCGCCTTTTGCGTTC | 476 |
| 11637 | TCCTGGCCGCCTTTTGCGTT | 475 |
| 11638 | GTCCTGGCCGCCTTTTGCGT | 474 |
| 11639 | TGTCCTGGCCGCCTTTTGCG | 473 |
| 11640 | CTGTCCTGGCCGCCTTTTGC | 472 |
| 11641 | GCTGTCCTGGCCGCCTTTTG | 471 |
| 11642 | CGCTGTCCTGGCCGCCTTTT | 470 |
| 11643 | ACGCTGTCCTGGCCGCCTTT | 469 |
| 11644 | CACGCTGTCCTGGCCGCCTT | 468 |
| 11645 | GCACGCTGTCCTGGCCGCCT | 467 |
| 11646 | TGCACGCTGTCCTGGCCGCC | 466 |
| 11647 | CTGCACGCTGTCCTGGCCGC | 465 |
| 11648 | GCTGCACGCTGTCCTGGCCG | 464 |
| 11649 | TGCTGCACGCTGTCCTGGCC | 463 |
| 11650 | CTGCTGCACGCTGTCCTGGC | 462 |
| 11651 | GCTGCTGCACGCTGTCCTGG | 461 |
| 11652 | AGCTGCTGCACGCTGTCCTG | 460 |
| 11653 | CAGCTGCTGCACGCTGTCCT | 459 |
| 11654 | CCAGCTGCTGCACGCTGTCC | 458 |
| 11655 | CCCAGCTGCTGCACGCTGTC | 457 |
| 11656 | TCCCAGCTGCTGCACGCTGT | 456 |
| 11657 | CTCCCAGCTGCTGCACGCTG | 455 |
| 11658 | GCTCCCAGCTGCTGCACGCT | 454 |
| 11659 | GGCTCCCAGCTGCTGCACGC | 453 |
| 11660 | CGGCTCCCAGCTGCTGCACG | 452 |
| 11661 | GCGGCTCCCAGCTGCTGCAC | 451 |
| 11662 | GGCGGCTCCCAGCTGCTGCA | 450 |
| 11663 | CGGCGGCTCCCAGCTGCTGC | 449 |
| 11664 | ACGGCGGCTCCCAGCTGCTG | 448 |
| 11665 | AACGGCGGCTCCCAGCTGCT | 447 |
| 11666 | GAACGGCGGCTCCCAGCTGC | 446 |
| 11667 | AGAACGGCGGCTCCCAGCTG | 445 |
| 11668 | GAGAACGGCGGCTCCCAGCT | 444 |
| 11669 | TGAGAACGGCGGCTCCCAGC | 443 |
| 11670 | CTGAGAACGGCGGCTCCCAG | 442 |
| 11671 | GCTGAGAACGGCGGCTCCCA | 441 |
| 11672 | GGCTGAGAACGGCGGCTCCC | 440 |
| 11673 | AGGCTGAGAACGGCGGCTCC | 439 |
| 11674 | AAGGCTGAGAACGGCGGCTC | 438 |
| 11675 | TAAGGCTGAGAACGGCGGCT | 437 |
| 11676 | TTAAGGCTGAGAACGGCGGC | 436 |
| 11677 | TTTAAGGCTGAGAACGGCGG | 435 |
| 11678 | TTTTAAGGCTGAGAACGGCG | 434 |
| 11679 | CTTTTAAGGCTGAGAACGGC | 433 |
| 11680 | ACTTTTAAGGCTGAGAACGG | 432 |
| 11681 | CGCAGGCACTGGAGCGGCCCCGGCG | 701 |
| 11682 | GCAGGCACTGGAGCGGCCCC | 702 |
| 11683 | CAGGCACTGGAGCGGCCCCG | 703 |
| 11684 | AGGCACTGGAGCGGCCCCGG | 704 |
| 11685 | GGCACTGGAGCGGCCCCGGC | 705 |
| 11686 | GCACTGGAGCGGCCCCGGCG | 706 |
| 11687 | CACTGGAGCGGCCCCGGCGG | 707 |
| 11688 | ACTGGAGCGGCCCCGGCGGC | 708 |
| 11689 | CTGGAGCGGCCCCGGCGGCA | 709 |
| 11690 | TGGAGCGGCCCCGGCGGCAG | 710 |
| 11691 | GGAGCGGCCCCGGCGGCAGC | 711 |
| 11692 | GAGCGGCCCCGGCGGCAGCA | 712 |
| 11693 | AGCGGCCCCGGCGGCAGCAG | 713 |
| 11694 | GCGGCCCCGGCGGCAGCAGC | 714 |
| 11695 | CGGCCCCGGCGGCAGCAGCT | 715 |
| 11696 | GGCCCCGGCGGCAGCAGCTG | 716 |
| 11697 | GCCCCGGCGGCAGCAGCTGA | 717 |
| 11698 | CCCCGGCGGCAGCAGCTGAG | 718 |
| 11699 | CCCGGCGGCAGCAGCTGAGG | 719 |
| 11700 | CCGGCGGCAGCAGCTGAGGG | 720 |
| 11701 | CGGCGGCAGCAGCTGAGGGC | 721 |
| 11702 | TCGCAGGCACTGGAGCGGCC | 700 |
| 11703 | TTCGCAGGCACTGGAGCGGC | 699 |
| 11704 | GTTCGCAGGCACTGGAGCGG | 698 |
| 11705 | AGTTCGCAGGCACTGGAGCG | 697 |
| 11706 | GAGTTCGCAGGCACTGGAGC | 696 |
| 11707 | AGAGTTCGCAGGCACTGGAG | 695 |
| 11708 | CAGAGTTCGCAGGCACTGGA | 694 |
| 11709 | TCAGAGTTCGCAGGCACTGG | 693 |
| 11710 | CTCAGAGTTCGCAGGCACTG | 692 |
| 11711 | CCTCAGAGTTCGCAGGCACT | 691 |
| 11712 | TCCTCAGAGTTCGCAGGCAC | 690 |
| 11713 | CTCCTCAGAGTTCGCAGGCA | 689 |
| 11714 | GCTCCTCAGAGTTCGCAGGC | 688 |
| 11715 | GGCTCCTCAGAGTTCGCAGG | 687 |
| 11716 | CGGCTCCTCAGAGTTCGCAG | 686 |
| 11717 | TCGGCTCCTCAGAGTTCGCA | 685 |
| 11718 | CTCGGCTCCTCAGAGTTCGC | 684 |
| 11719 | CCTCGGCTCCTCAGAGTTCG | 683 |
| 11720 | GCCTCGGCTCCTCAGAGTTC | 682 |
| 11721 | CGCCTCGGCTCCTCAGAGTT | 681 |
| 11722 | GCGCCTCGGCTCCTCAGAGT | 680 |
| 11723 | GGCGCCTCGGCTCCTCAGAG | 679 |
| 11724 | CGGCGCCTCGGCTCCTCAGA | 678 |
| 11725 | CCGGCGCCTCGGCTCCTCAG | 677 |
| 11726 | ACCGGCGCCTCGGCTCCTCA | 676 |
| 11727 | CACCGGCGCCTCGGCTCCTC | 675 |
| 11728 | TCACCGGCGCCTCGGCTCCT | 674 |
| 11729 | CTCACCGGCGCCTCGGCTCC | 673 |
| 11730 | TCTCACCGGCGCCTCGGCTC | 672 |
| 11731 | CTCTCACCGGCGCCTCGGCT | 671 |
| 11732 | GCTCTCACCGGCGCCTCGGC | 670 |
| 11733 | TGCTCTCACCGGCGCCTCGG | 669 |
| 11734 | TTGCTCTCACCGGCGCCTCG | 668 |
| 11735 | CTTGCTCTCACCGGCGCCTC | 667 |
| 11736 | CCTTGCTCTCACCGGCGCCT | 666 |
| 11737 | TCCTTGCTCTCACCGGCGCC | 665 |
| 11738 | GTCCTTGCTCTCACCGGCGC | 664 |
| 11739 | CGTCCTTGCTCTCACCGGCG | 663 |
| 11740 | GCGTCCTTGCTCTCACCGGC | 662 |
| 11741 | AGCGTCCTTGCTCTCACCGG | 661 |
| 11742 | CAGCGTCCTTGCTCTCACCG | 660 |
| 11743 | GCAGCGTCCTTGCTCTCACC | 659 |
| 11744 | TGCAGCGTCCTTGCTCTCAC | 658 |
| 11745 | TTGCAGCGTCCTTGCTCTCA | 657 |
| 11746 | TTTGCAGCGTCCTTGCTCTC | 656 |
| 11747 | GTTTGCAGCGTCCTTGCTCT | 655 |
| 11748 | AGTTTGCAGCGTCCTTGCTC | 654 |
| 11749 | AAGTTTGCAGCGTCCTTGCT | 653 |
| 11750 | CAAGTTTGCAGCGTCCTTGC | 652 |
| 11751 | GCAAGTTTGCAGCGTCCTTG | 651 |
| 11752 | CGCAAGTTTGCAGCGTCCTT | 650 |
| 11753 | GCGCAAGTTTGCAGCGTCCT | 649 |
| 11754 | TGCGCAAGTTTGCAGCGTCC | 648 |
| 11755 | CTGCGCAAGTTTGCAGCGTC | 647 |
| 11756 | GCTGCGCAAGTTTGCAGCGT | 646 |
| 11757 | CGCTGCGCAAGTTTGCAGCG | 645 |
| 11758 | GCGCTGCGCAAGTTTGCAGC | 644 |
| 11759 | CGCGCTGCGCAAGTTTGCAG | 643 |
| 11760 | CCGCGCTGCGCAAGTTTGCA | 642 |
| 11761 | CCCGCGCTGCGCAAGTTTGC | 641 |
| 11762 | CCCCGCGCTGCGCAAGTTTG | 640 |
| 11763 | CCCCCGCGCTGCGCAAGTTT | 639 |
| 11764 | GCCCCCGCGCTGCGCAAGTT | 638 |
| 11765 | AGCCCCCGCGCTGCGCAAGT | 637 |
| 11766 | CAGCCCCCGCGCTGCGCAAG | 636 |
| 11767 | CCAGCCCCCGCGCTGCGCAA | 635 |
| 11768 | CCCAGCCCCCGCGCTGCGCA | 634 |
| 11769 | TCCCAGCCCCCGCGCTGCGC | 633 |
| 11770 | ATCCCAGCCCCCGCGCTGCG | 632 |
| 11771 | AATCCCAGCCCCCGCGCTGC | 631 |
| 11772 | GAATCCCAGCCCCCGCGCTG | 630 |
| 11773 | TGAATCCCAGCCCCCGCGCT | 629 |
| 11774 | GTGAATCCCAGCCCCCGCGC | 628 |
| 11775 | CGTGAATCCCAGCCCCCGCG | 627 |
| 11776 | GCGTGAATCCCAGCCCCCGC | 626 |
| 11777 | GGCGTGAATCCCAGCCCCCG | 625 |
| 11778 | GGGCGTGAATCCCAGCCCCC | 624 |
| 11779 | TGGGCGTGAATCCCAGCCCC | 623 |
| 11780 | CTGGGCGTGAATCCCAGCCC | 622 |
| 11781 | TCTGGGCGTGAATCCCAGCC | 621 |
| 11782 | TTCTGGGCGTGAATCCCAGC | 620 |
| 11783 | CTTCTGGGCGTGAATCCCAG | 619 |
| 11784 | ACTTCTGGGCGTGAATCCCA | 618 |
| 11785 | AACTTCTGGGCGTGAATCCC | 617 |
| 11786 | GAACTTCTGGGCGTGAATCC | 616 |
| 11787 | TGAACTTCTGGGCGTGAATC | 615 |
| 11788 | CTGAACTTCTGGGCGTGAAT | 614 |
| 11789 | GCTGAACTTCTGGGCGTGAA | 613 |
| 11790 | TGCTGAACTTCTGGGCGTGA | 612 |
| 11791 | CTGCTGAACTTCTGGGCGTG | 611 |
| 11792 | CCTGCTGAACTTCTGGGCGT | 610 |
| 11793 | GCCTGCTGAACTTCTGGGCG | 609 |
| 11794 | CGACCCTCCGAACAGAGGCGGCGGG | 851 |
| 11795 | GACCCTCCGAACAGAGGCGG | 852 |
| 11796 | ACCCTCCGAACAGAGGCGGC | 853 |
| 11797 | CCCTCCGAACAGAGGCGGCG | 854 |
| 11798 | CCTCCGAACAGAGGCGGCGG | 855 |
| 11799 | CTCCGAACAGAGGCGGCGGG | 856 |
| 11800 | GCGACCCTCCGAACAGAGGC | 850 |
| 11801 | CGCGACCCTCCGAACAGAGG | 849 |
| 11802 | CCGCGACCCTCCGAACAGAG | 848 |
| 11803 | CCCGCGACCCTCCGAACAGA | 847 |
| 11804 | CCCCGCGACCCTCCGAACAG | 846 |
| 11805 | GCCCCGCGACCCTCCGAACA | 845 |
| 11806 | TGCCCCGCGACCCTCCGAAC | 844 |
| 11807 | GTGCCCCGCGACCCTCCGAA | 843 |
| 11808 | GGTGCCCCGCGACCCTCCGA | 842 |
| 11809 | CGGTGCCCCGCGACCCTCCG | 841 |
| 11810 | TCGGTGCCCCGCGACCCTCC | 840 |
| 11811 | CTCGGTGCCCCGCGACCCTC | 839 |
| 11812 | CCTCGGTGCCCCGCGACCCT | 838 |
| 11813 | ACCTCGGTGCCCCGCGACCC | 837 |
| 11814 | CACCTCGGTGCCCCGCGACC | 836 |
| 11815 | GCACCTCGGTGCCCCGCGAC | 835 |
| 11816 | AGCACCTCGGTGCCCCGCGA | 834 |
| 11817 | AAGCACCTCGGTGCCCCGCG | 833 |
| 11818 | AAAGCACCTCGGTGCCCCGC | 832 |
| 11819 | GAAAGCACCTCGGTGCCCCG | 831 |
| 11820 | GGAAAGCACCTCGGTGCCCC | 830 |
| 11821 | CGGAAAGCACCTCGGTGCCC | 829 |
| 11822 | CCGGAAAGCACCTCGGTGCC | 828 |
| 11823 | GCCGGAAAGCACCTCGGTGC | 827 |
| 11824 | GGCCGGAAAGCACCTCGGTG | 826 |
| 11825 | CGCGCTGCTGCCCGACCCACGCAGT | 1022 |
| 11826 | GCGCTGCTGCCCGACCCACG | 1023 |
| 11827 | CGCTGCTGCCCGACCCACGC | 1024 |
| 11828 | GCTGCTGCCCGACCCACGCA | 1025 |
| 11829 | CTGCTGCCCGACCCACGCAG | 1026 |
| 11830 | TGCTGCCCGACCCACGCAGT | 1027 |
| 11831 | GCTGCCCGACCCACGCAGTC | 1028 |
| 11832 | CTGCCCGACCCACGCAGTCC | 1029 |
| 11833 | TGCCCGACCCACGCAGTCCG | 1030 |
| 11834 | GCCCGACCCACGCAGTCCGG | 1031 |
| 11835 | CCCGACCCACGCAGTCCGGC | 1032 |
| 11836 | CCGACCCACGCAGTCCGGCC | 1033 |
| 11837 | CGACCCACGCAGTCCGGCCT | 1034 |
| 11838 | GACCCACGCAGTCCGGCCTC | 1035 |
| 11839 | ACCCACGCAGTCCGGCCTCG | 1036 |
| 11840 | CCCACGCAGTCCGGCCTCGC | 1037 |
| 11841 | CCACGCAGTCCGGCCTCGCC | 1038 |
| 11842 | CACGCAGTCCGGCCTCGCCC | 1039 |
| 11843 | ACGCAGTCCGGCCTCGCCCC | 1040 |
| 11844 | CGCAGTCCGGCCTCGCCCCG | 1041 |
| 11845 | GCAGTCCGGCCTCGCCCCGC | 1042 |
| 11846 | CAGTCCGGCCTCGCCCCGCC | 1043 |
| 11847 | AGTCCGGCCTCGCCCCGCCC | 1044 |
| 11848 | GTCCGGCCTCGCCCCGCCCC | 1045 |
| 11849 | TCCGGCCTCGCCCCGCCCCA | 1046 |
| 11850 | CCGGCCTCGCCCCGCCCCAC | 1047 |
| 11851 | CGGCCTCGCCCCGCCCCACC | 1048 |
| 11852 | GGCCTCGCCCCGCCCCACCC | 1049 |
| 11853 | GCCTCGCCCCGCCCCACCCG | 1050 |
| 11854 | CCTCGCCCCGCCCCACCCGC | 1051 |
| 11855 | CTCGCCCCGCCCCACCCGCA | 1052 |
| 11856 | TCGCCCCGCCCCACCCGCAC | 1053 |
| 11857 | CGCCCCGCCCCACCCGCACC | 1054 |
| 11858 | GCCCCGCCCCACCCGCACCC | 1055 |
| 11859 | CCCCGCCCCACCCGCACCCT | 1056 |
| 11860 | CCCGCCCCACCCGCACCCTC | 1057 |
| 11861 | CCGCCCCACCCGCACCCTCC | 1058 |
| 11862 | CGCCCCACCCGCACCCTCCA | 1059 |
| 11863 | GCCCCACCCGCACCCTCCAA | 1060 |
| 11864 | CCCCACCCGCACCCTCCAAC | 1061 |
| 11865 | CCCACCCGCACCCTCCAACC | 1062 |
| 11866 | CCACCCGCACCCTCCAACCA | 1063 |
| 11867 | CACCCGCACCCTCCAACCAA | 1064 |
| 11868 | ACCCGCACCCTCCAACCAAT | 1065 |
| 11869 | CCCGCACCCTCCAACCAATG | 1066 |
| 11870 | CCGCACCCTCCAACCAATGG | 1067 |
| 11871 | CGCACCCTCCAACCAATGGC | 1068 |
| 11872 | GCACCCTCCAACCAATGGCG | 1069 |
| 11873 | CACCCTCCAACCAATGGCGT | 1070 |
| 11874 | ACCCTCCAACCAATGGCGTG | 1071 |
| 11875 | CCCTCCAACCAATGGCGTGG | 1072 |
| 11876 | CCTCCAACCAATGGCGTGGC | 1073 |
| 11877 | CTCCAACCAATGGCGTGGCT | 1074 |
| 11878 | TCCAACCAATGGCGTGGCTC | 1075 |
| 11879 | CCAACCAATGGCGTGGCTCG | 1076 |
| 11880 | CAACCAATGGCGTGGCTCGA | 1077 |
| 11881 | AACCAATGGCGTGGCTCGAT | 1078 |
| 11882 | ACCAATGGCGTGGCTCGATC | 1079 |
| 11883 | CCGCGCTGCTGCCCGACCCA | 1021 |
| 11884 | TCCGCGCTGCTGCCCGACCC | 1020 |
| 11885 | CTCCGCGCTGCTGCCCGACC | 1019 |
| 11886 | ACTCCGCGCTGCTGCCCGAC | 1018 |
| 11887 | AACTCCGCGCTGCTGCCCGA | 1017 |
| 11888 | CAACTCCGCGCTGCTGCCCG | 1016 |
| 11889 | CCAACTCCGCGCTGCTGCCC | 1015 |
| 11890 | GCCAACTCCGCGCTGCTGCC | 1014 |
| 11891 | AGCCAACTCCGCGCTGCTGC | 1013 |
| 11892 | AAGCCAACTCCGCGCTGCTG | 1012 |
| 11893 | CAAGCCAACTCCGCGCTGCT | 1011 |
| 11894 | ACAAGCCAACTCCGCGCTGC | 1010 |
| 11895 | CACAAGCCAACTCCGCGCTG | 1009 |
| 11896 | TCACAAGCCAACTCCGCGCT | 1008 |
| 11897 | CTCACAAGCCAACTCCGCGC | 1007 |
| 11898 | GCTCACAAGCCAACTCCGCG | 1006 |
| 11899 | GGCTCACAAGCCAACTCCGC | 1005 |
| 11900 | GGGCTCACAAGCCAACTCCG | 1004 |
| 11901 | CGGGTCCGACTTCGGAAAAACAGGT | 1313 |
| 11902 | GGGTCCGACTTCGGAAAAAC | 1314 |
| 11903 | GGTCCGACTTCGGAAAAACA | 1315 |
| 11904 | GTCCGACTTCGGAAAAACAG | 1316 |
| 11905 | TCCGACTTCGGAAAAACAGG | 1317 |
| 11906 | CCGACTTCGGAAAAACAGGT | 1318 |
| 11907 | CGACTTCGGAAAAACAGGTT | 1319 |
| 11908 | GACTTCGGAAAAACAGGTTC | 1320 |
| 11909 | ACTTCGGAAAAACAGGTTCC | 1321 |
| 11910 | CTTCGGAAAAACAGGTTCCA | 1322 |
| 11911 | TTCGGAAAAACAGGTTCCAG | 1323 |
| 11912 | TCGGAAAAACAGGTTCCAGA | 1324 |
| 11913 | ACGGGTCCGACTTCGGAAAA | 1312 |
| 11914 | AACGGGTCCGACTTCGGAAA | 1311 |
| 11915 | AAACGGGTCCGACTTCGGAA | 1310 |
| 11916 | TAAACGGGTCCGACTTCGGA | 1309 |
| 11917 | TTAAACGGGTCCGACTTCGG | 1308 |
| 11918 | ATTAAACGGGTCCGACTTCG | 1307 |
| 11919 | GATTAAACGGGTCCGACTTC | 1306 |
| 11920 | AGATTAAACGGGTCCGACTT | 1305 |
| 11921 | GAGATTAAACGGGTCCGACT | 1304 |
| 11922 | AGAGATTAAACGGGTCCGAC | 1303 |
| 11923 | CGGCCTGTCGGGTAGCACAGGAGTT | 2022 |
| 11924 | GGCCTGTCGGGTAGCACAGG | 2023 |
| 11925 | GCCTGTCGGGTAGCACAGGA | 2024 |
| 11926 | CCTGTCGGGTAGCACAGGAG | 2025 |
| 11927 | CTGTCGGGTAGCACAGGAGT | 2026 |
| 11928 | TGTCGGGTAGCACAGGAGTT | 2027 |
| 11929 | GTCGGGTAGCACAGGAGTTT | 2028 |
| 11930 | TCGGGTAGCACAGGAGTTTT | 2029 |
| 11931 | CGGGTAGCACAGGAGTTTTC | 2030 |
| 11932 | ACGGCCTGTCGGGTAGCACA | 2021 |
| 11933 | CACGGCCTGTCGGGTAGCAC | 2020 |
| 11934 | TCACGGCCTGTCGGGTAGCA | 2019 |
| 11935 | CTCACGGCCTGTCGGGTAGC | 2018 |
| 11936 | GCTCACGGCCTGTCGGGTAG | 2017 |
| 11937 | AGCTCACGGCCTGTCGGGTA | 2016 |
| 11938 | GAGCTCACGGCCTGTCGGGT | 2015 |
| 11939 | GGAGCTCACGGCCTGTCGGG | 2014 |
| 11940 | TGGAGCTCACGGCCTGTCGG | 2013 |
| 11941 | CTGGAGCTCACGGCCTGTCG | 2012 |
| 11942 | TCTGGAGCTCACGGCCTGTC | 2011 |
| 11943 | CTCTGGAGCTCACGGCCTGT | 2010 |
| 11944 | TCTCTGGAGCTCACGGCCTG | 2009 |
| 11945 | CTCTCTGGAGCTCACGGCCT | 2008 |
| 11946 | CCTCTCTGGAGCTCACGGCC | 2007 |
| 11947 | TCCTCTCTGGAGCTCACGGC | 2006 |
| 11948 | ATCCTCTCTGGAGCTCACGG | 2005 |
| 11949 | GATCCTCTCTGGAGCTCACG | 2004 |

Hot Zones (Relative upstream location to gene start site)
1-550
650-950
1000-1100
1250-1400
1950-2100

### Examples

Genetic Code (5′ Upstream Region)

(SEQ ID NO: 11986)

TCTCTGCAGCCCCCTAGTGGCCATTGGGTGCAGCAGACGATTCACAGTTA
ACTGACAAATTAACTGGAGTCAGTAATGCCTTTGGTCAAGAATTGTATAG
AGAAATAGGGAAAGGCTGGAGTTTTAGTCTTTTTTCATATTTCAAATAAA
AATTCCTCTTCCAGTAGGTATGTCAGAAAAATCTGATGAAAATCAAACAT
ATATTGTACCAGGAAAGTATTAACTACCATAGCATTTTCCTCCCTCTTTT
CTTTCTTTTCCACCCTTCCTCCACCAAGATAGGAGCATATTTTCTTCTCG
GGTGAGATAATTCTTTGCCCTGAAACTTGTAAAGTCAGTGTATCCAGTGT
GACTTCCAGAGAGAGGGCAGATGCCTGTCAAATTAAGTGAGTTGCCAAAC
ATAGAGCAGGAAGAAAGCCATTCCGAGAATCAATATTCCTTTGTTACTGG
GTCTTCCACTTGCCAAGGCATTGCCACAAAGCTGGAAAGGCCCAGCTCCT
AGGAGAACAGAGGTTCCACCTGGCCACTATCTCCTGTGGGGTGGTAGGCA
AGTTACTGCGGCCCCCAGGAGCTCAGTGAGGGAGGTTCAATGTGACACTG
TGCTCTGATCCTGTGAGAAAACTCCTGTGCTACCCGACAGGCCGTGAGCT
CCAGAGAGGATCTTGCCTTATTCTTAGCTTCAACAGTCAGCCCAAGGCCT
GACAACCAGCCTTTAAGAAGGAATCAAGGGGATTTGTGTGACCCAAAGAT
GGTAGTTTTGTCTGAGGATCTAGTGAACCACTTGTTATAAAAACAGCTAT
TATGAGTTCTGTGTTGGCAGCTCAGGAGAGACGAAAGGAAAGGGAGAGGA
GAGGTACAGCCATTACAGGTGAGTAAAAAAGGCCTAAGGTTCTGAACCCT
CATTCCCAAGATTGTGGGCAAACAATTAAATGCTCTGCAACTCAGTTTCT
GCATCTGTAAATCTGGAATTAAAATGTTTGCCTTACAGAGACTAGGGGAG
GTTACACATGTTCAGACACCATTCTGAGAAAACAGAGCGACTGACAGGGG
TCTGAAAGGTATTTGTTGTAGCTGCAGAACAACTCTGCCAGACCAAGACC
ATCCATCCCTCTCTGCCCCCCTATTCCCAAATTCTCCTGTGTGGACGGCA
GGACTCCTAAGCTCCCAGGAATGCATTCAAATAATAGATGGGTCAGAAAA
TATTCTGTCTCAGGGCCTTAATACAAGCTGTTCTCAGATTTGCCAGTGTC
GCGCTGCCACCCTCTCCCCACTTCCTCCTCCCTTCCCACTCCCCCCTCCC
TTCCCCTCTCCTCCAGTTTTATTCTGGAACCTGTTTTTCCGAAGTCGGAC
CCGTTTAATCTCTTAAATGTATAATTAGGGAGAGTGCTTGATTGCAAAGG
CCTCTTCCAGTTCTCACATTTGCTCCCTTTCACACTGCAGAGAAATAGGG
CAGGGAATCTAGAGGAGGGGAAGAACAAGAGACTGGAGAGGGAACAGAGG
GAGGGTGGGGCGGGCTCACTCCTTTTCTCAATGAATGCCGAGGCCTCTGC
AGATTTGCATAGGAGCCGATCGAGCCACGCCATTGGTTGGAGGGTGCGGG
TGGGGCGGGGCGAGGCCGGACTGCGTGGGTCGGGCAGCAGCGCGGAGTTG
GCTTGTGAGCCCCGCCCCCTCCGGGCCCCGCCCCCTCCCTGCGCGCGCTC
GCGCGGCTCAGCCAGCTGCAAGTGGCGGGCGCCCAGGCAGATGCGATCCA
GCGGCTCTGGGGGCGGCAGCGGTGGTAGCAGCTGGTACCTCCCGCCGCCT
CTGTTCGGAGGGTCGCGGGGCACCGAGGTGCTTTCCGGCCGCCCTCTGGT
CGGCCACCCAAAGCCGCGGGCGCTGATGATGGGTGAGGAGGGGGCGGCAA
GATTTCGGGCGCCCCTGCCCTGAACGCCCTCAGCTGCTGCCGCCGGGGCC
GCTCCAGTGCCTGCGAACTCTGAGGAGCCGAGGCGCCGGTGAGAGCAAGG
ACGCTGCAAACTTGCGCAGCGCGGGGGCTGGGATTCACGCCCAGAAGTTC
AGCAGGCAGACAGTCCGAAGCCTTCCCGCAGCGGAGAGATAGCTTGAGGG
TGCGCAAGACGGCAGCCTCCGCCCTCGGTTCCCGCCCAGACCGGGCAGAA
GAGCTTGGAGGAGCCAAAAGGAACGCAAAAGGCGGCCAGGACAGCGTGCA
GCAGCTGGGAGCCGCCGTTCTCAGCCTTAAAAGTTGCAGAGATTGGAGGC
TGCCCCGAGAGGGGACAGACCCCAGCTCCGACTGCGGGGGGCAGGAGAGG
ACGGTACCCAACTGCCACCTCCCTTCAACCATAGTAGTTCCTCTGTACCG
AGCGCAGCGAGCTACAGACGGGGGCGCGGCACTCGGCGCGGAGAGCGGGA
GGCTCAAGGTCCCAGCCAGTGAGCCCAGTGTGCTTGAGTGTCTCTGGACT
CGCCCCTGAGCTTCCAGGTCTGTTTCATTTAGACTCCTGCTCGCCTCCGT
GCAGTTGGGGGAAAGCAAGAGACTTGCGCGCACGCACAGTCCTCTGGAGA
TCAGGTGGAAGGAGCCGCTGGGTACCAAGGACTGTTCAGAGCCTCTTCCC
ATCTCGGGGAGAGCGAAGGGTGAGGCTGGGCCCGGAGAGCAGTGTAAACG
GCCTCCTCCGGCGGGA
TG

Musashi Homolog 2 (MSI2)

Musashi homolog 2 is located on chromosome 17 and belongs to RNA-binding proteins of the Musashi family expressed in stem cell compartments and in aggressive tumors. MSI2 is the predominant form expressed in hematopoietic stem cells (HSCs), and its knockdown leads to reduced engraftment and depletion of HSCs in vivo. Overexpression of human MSI2 in a mouse model increases HSC cell cycle progression and cooperates with the chronic myeloid leukemia-associated BCR-ABL1 oncoprotein to induce an aggressive leukemia. MSI2 is overexpressed in human myeloid leukemia cell lines, and its depletion leads to decreased proliferation and increased apoptosis. Expression levels in human myeloid leukemia directly correlate with decreased survival in patients with the disease.

Protein: MSI2 Gene: MSI2 (Homo sapiens, chromosome 17, 57256570-57684689 [NCBI Reference Sequence: NC_{—}000017.11]; start site location: 57256743; strand: positive)

Gene Identification

| | |
|---|---|
| GeneID | 124540 |
| HGNC | 18585 |
| HPRD | 07438 |
| MIM | 607897 |

Targeted Sequences

| | | Relative |
|---|---|---|
| | | upstream |
| | | location |
| Sequence | | to gene |
| ID No: | Sequence (5′-3′) | start site |
| 11989 | CGGTGACGTCACGCACCCCCGTGCG | 360 |
| 12058 | CGGATACAATTACCCATATTGT | 1535 |
| 12059 | GACTCAGTTGCTAACAACCATGAGCG | 10624 |
| 12060 | CAGTTGCTAACAACCATGAGCG | 10628 |
| 12061 | CATGAAAATTTCACCAAGTATAAATTAC | 10909 |
| 12062 | CACCAAGTATAAATTACAGGTCT | 10920 |

Targeted Shift Sequences

| | | Relative |
|---|---|---|
| | | upstream |
| | | location |
| Sequence | | to gene |
| ID No: | Sequence (5′-3′) | start site |
| 11989 | CGGTGACGTCACGCACCCCCGTGCG | 354 |
| 11990 | GGTGACGTCACGCACCCCCG | 355 |
| 11991 | GTGACGTCACGCACCCCCGT | 356 |
| 11992 | TGACGTCACGCACCCCCGTG | 357 |
| 11993 | GACGTCACGCACCCCCGTGC | 358 |
| 11994 | ACGTCACGCACCCCCGTGCG | 359 |
| 11995 | CGTCACGCACCCCCGTGCGG | 360 |
| 11996 | GTCACGCACCCCCGTGCGGC | 361 |
| 11997 | TCACGCACCCCCGTGCGGCC | 362 |
| 11998 | CACGCACCCCCGTGCGGCCC | 363 |
| 11999 | ACGCACCCCCGTGCGGCCCC | 364 |
| 12000 | CGCACCCCCGTGCGGCCCCC | 365 |
| 12001 | GCACCCCCGTGCGGCCCCCG | 366 |
| 12002 | CACCCCCGTGCGGCCCCCGC | 367 |
| 12003 | ACCCCCGTGCGGCCCCCGCC | 368 |
| 12004 | CCCCCGTGCGGCCCCCGCCT | 369 |
| 12005 | CCCCGTGCGGCCCCCGCCTG | 370 |
| 12006 | CCCGTGCGGCCCCCGCCTGC | 371 |
| 12007 | CCGTGCGGCCCCCGCCTGCC | 372 |
| 12008 | CGTGCGGCCCCCGCCTGCCC | 373 |
| 12009 | GTGCGGCCCCCGCCTGCCCG | 374 |
| 12010 | TGCGGCCCCCGCCTGCCCGC | 375 |
| 12011 | GCGGCCCCCGCCTGCCCGCG | 376 |
| 12012 | CGGCCCCCGCCTGCCCGCGC | 377 |
| 12013 | GGCCCCCGCCTGCCCGCGCG | 378 |
| 12014 | GCCCCCGCCTGCCCGCGCGC | 379 |
| 12015 | CCCCCGCCTGCCCGCGCGCG | 380 |
| 12016 | CCCCGCCTGCCCGCGCGCGC | 381 |
| 12017 | CCCGCCTGCCCGCGCGCGCA | 382 |
| 12018 | CCGCCTGCCCGCGCGCGCAC | 383 |
| 12019 | CGCCTGCCCGCGCGCGCACA | 384 |
| 12020 | GCCTGCCCGCGCGCGCACAC | 385 |
| 12021 | CCTGCCCGCGCGCGCACACT | 386 |
| 12022 | CTGCCCGCGCGCGCACACTC | 387 |
| 12023 | TGCCCGCGCGCGCACACTCG | 388 |
| 12024 | GCCCGCGCGCGCACACTCGG | 389 |
| 12025 | CCCGCGCGCGCACACTCGGC | 390 |
| 12026 | CCGCGCGCGCACACTCGGCC | 391 |
| 12027 | CGCGCGCGCACACTCGGCCC | 392 |
| 12028 | GCGCGCGCACACTCGGCCCC | 393 |
| 12029 | CGCGCGCACACTCGGCCCCC | 394 |
| 12030 | GCGCGCACACTCGGCCCCCC | 395 |
| 12031 | CGCGCACACTCGGCCCCCCA | 396 |
| 12032 | GCGCACACTCGGCCCCCCAC | 397 |
| 12033 | CGCACACTCGGCCCCCCACG | 398 |
| 12034 | GCACACTCGGCCCCCCACGG | 399 |
| 12035 | CACACTCGGCCCCCCACGGC | 400 |
| 12036 | ACACTCGGCCCCCCACGGCC | 401 |
| 12037 | CCGGTGACGTCACGCACCCC | 353 |
| 12038 | GCCGGTGACGTCACGCACCC | 352 |
| 12039 | TGCCGGTGACGTCACGCACC | 351 |
| 12040 | ATGCCGGTGACGTCACGCAC | 350 |
| 12041 | AATGCCGGTGACGTCACGCA | 349 |
| 12042 | CAATGCCGGTGACGTCACGC | 348 |
| 12043 | CCAATGCCGGTGACGTCACG | 347 |
| 12044 | ACCAATGCCGGTGACGTCAC | 346 |
| 12045 | AACCAATGCCGGTGACGTCA | 345 |
| 12046 | TAACCAATGCCGGTGACGTC | 344 |
| 12047 | GTAACCAATGCCGGTGACGT | 343 |
| 12048 | TGTAACCAATGCCGGTGACG | 342 |
| 12049 | GTGTAACCAATGCCGGTGAC | 341 |
| 12050 | CGTGTAACCAATGCCGGTGA | 340 |
| 12051 | TCGTGTAACCAATGCCGGTG | 339 |
| 12052 | GTCGTGTAACCAATGCCGGT | 338 |
| 12053 | CGTCGTGTAACCAATGCCGG | 337 |
| 12054 | ACGTCGTGTAACCAATGCCG | 336 |
| 12055 | AACGTCGTGTAACCAATGCC | 335 |
| 12056 | GAACGTCGTGTAACCAATGC | 334 |
| 12057 | AGAACGTCGTGTAACCAATG | 333 |
| 12058 | CGGATACAATTACCCATATTGT | 1535 |
| 12059 | GACTCAGTTGCTAACAACCATGAGCG | 10624 |
| 12060 | CAGTTGCTAACAACCATGAGCG | 10628 |
| 12061 | CATGAAAATTTCACCAAGTATAAATTAC | 10909 |
| 12062 | CACCAAGTATAAATTACAGGTCT | 10920 |

Hot Zones (Relative upstream location to gene start site)
1-450
1450-1600
10000-11500

### Examples

Genetic Code (5′ Upstream Region)

(SEQ ID NO: 1364)

ATTTCTCAAAGAACTAAAAATAGAACTGCCATTTGATCCAGCAATCCCAC
TACTGGTAACCTTTAACAGTATATACCCAAAGGAAAAGAAATCAGTATAT
CAAAAAGATACCCATACTCGTATGTTTATCGTAGCACTATTCACAATAGC
AAAGATATGGAATCAACCTAAGTGTCCATCAACAGAGGATTGGATAAAGA
AAATGTGATACATGTACACAATAAAGTACTACTCAGTCATTAAAAAAATC
AAACAGCAGCAATATGGATGGAATTGCTGGAAGACATTATCCCCAGGTGA
AACAAGCCGGAGACAGAAAGACAAACACTGCGTGTTTTCACTTATAAGTG
GGAGCTAAATCATGTGTACACATGGATGTAGGGTGTGGAATAACAGATAA
TGGAGACTTGAAAGAGTGAGGGGGCCAGGCATGGTGGCTCATGCCTGTAA
TCCCAGCACTTTGGGAGGCCGAGGTGGGTGGATCATCTGAGGTCAGGAGT
TTGAGACCAGCCTGGCCAACATGGTGAAACCTTGTCTCTACTAAAAATAC
AAAAAGTAGCCGGGCATGGTGGTGTGTGCCTGTAATCCTAGCTACTCAGG
AGGCTGAAGCAGGAGAATAGCTTGAACCCGGGAGGGTGGGAGGTTGCAGT
GAGCCGAGATCACGCCACTGCACTCCAGCCTGGGCAACAAGAGCGAAACT
CGGTCTCGAAAAAAGAAAAAAAAAAAAAAAAGAAAGGGTGAGGGGATGGG
GGAAGTGAATGATGAGAGACTACTTAATGGGTACAATGTATTTGAGTGAT
GAATACCCTAAAAACCCTGATTTTACCACTATGTGATCTATGCATGTAAC
AAAATTATACACGTAACTCATAAATTTACATAAATAAACTAAAAAATAAT
TTTTAAGTTAGCAAACAACTTTTTTAAAAGAAGAATAAGCAGATACCCCA
CATAGTGAGTAGACAAAGAACATCCCAGGCAAAAGGAACAAACAGCATCT
GCAAAGGGCTTGAAGAAGGAAACAGCTTGTTTTGTTTAAGAAATGATAGA
AGGCTGGGCGTGGTGGCTCACGCCTGTAATCCTAGCACTTTGAGAGGCCA
AGGAAGGTAGATTGCTTGAGCCTAAGAGTTTGGGACCAGCCTGGGCAACG
TGGCAAAACCGCCAAAATTAACCGGGCTTGGTGGCATGCAGCTGTAGTCC
CAGCTACTCAGGAGGCTGAGGTGGGAGGATCACCTGAGCCCAGGTGGTCA
AGGCTGTGGTGTGCTGTGATCATGCTACTGCACTCCAGCCTGGATGACAG
AGTGAGAACCTGTCTAAAAAATTAATTAATTAATTAAAAAAAATGAAAGA
GATGATAGAAGATGGTGGTGTAATGTGAGGTTGGAAAAGCAGACCTAGAA
CATACCACGGAAGGTCTTTTAGGTGACAGCAAGGGGGTTCGATGCAGTGG
GAAACCGCTGGAGGGATCCGACCTGCATCCCATAAAGACCTCTTTGGCTA
CTGTGAGGGAAACAGACATTTTGGGAAGGTTCCAGAAGTCAAGGTAGAGG
AAGACTAGTCTATAAAGCGGACCGCCTTTGTGAAAAATCAACCTATGAAA
GAAGCCAACATACAAAAGATACACTGGAAGTGATCAAAGACTTCCAAGAG
CAGCCGGGAGGTGGAATCTCAAGTCCAGATGTTGAATGAGTTGGGTGATT
GTATGGGACAGAACCAGAAGCTGATGAGGGGCCCAGGATGCAGATCAAAG
AGATGGGATGAGCAGGCAAATGCCATTTCTTTTCATTCCGCCTATTTTGC
TTGAGTCACCAGTTTGGTAAGGGGAGAGTTTCAACCATCTGCAAGTAATC
CAAATGCTTTTACTAACCTGCCTACCCATCCACACCCCCACCAAAAAAAA
AAAAAAAGAAAGAAAGAAAGAAAAATAAGAAGCCAACCCCAGAGCTTGTA
CGCCTGCATTCTCCACAATCATTTCTCTGTGTTACAGCTCTTGTCATCTT
ACATTATACATGAATAACTAATCAACCAAACACAAACCCACTGATGAAAA
AAAACGCATGTTGGATCTAGAATGTGAGCAGGGTAAAGAGTAATATTAAT
TTCACTGGCAAATAACAAAATGGGAGCAAACATGAGGGATGTTAAGACAA
GACTTGTGTTTTTGAATGCTTCTGGGAATGGAAACTCTCCAAGAATCAAG
GGAAAGGGGAAGAAGGTTGGACATATTTGGAGTGCACTAATACCAATTCT
TTTTTCTTCTGGTATGCATATGTGACTATATGCAACATCATCCTGGGTCA
GGCCAACTGGTCATTTAAACCACCATGCTGTCTGCCTCACCTAGTCGAAC
AGTTCATATAATGATCTTCCACTATTCCTTAATGGACATACAGGTGTTAG
TCTTAATGCACCAACCAATATTGCGATAGGCTGGAACAAAACCTTTTGCT
CTTTGAATGTCCACAGTGAGTAACACTATGCTAGGTATGTAGTAGGTGCT
CAATAAATGTATCTTTCACACACTTCCTAAATGAAGTCTATTTCTTCCTT
CATCGTTTTCCTGAAAGATCTTTCTTGACCACTAAGGATGAACTCCTCAT
CTCCATGTCCAGTGAGTGGTTTAGAAATGTTTTAGTCTTCCTCCTCCTTG
ACTTCATAATGGCATCTGACACTGCTGTGACACTTCAGTTGTCTTCTGTG
ACTCTGAACTTTTCTGGTTCTAATCTTTTCTCCCTGATGGCCTCTCTACC
CTGACCTCCAATGATTTCTTTTCCTGTTTCTGCACTCGAAAGTCCATATT
CCACAAGGACTCTGCTTTTCTGTCTTGTCACTCTTTATGAGAAATTAATT
GTATCTCATTTCAAATCAATGCATGTATCTATCATCCTCTGTCACATTAC
ATGCAAAGTATCTGCTTATGGATCTGTCTGTCCAACTAGACCTCAAGCTC
CTCAAAAACAGATCCCTCACTGTGTTCGTCTCTGTGCCTCCAGTACCTGG
CACAGAGCTGGACACTCAGTGAATGCTCCATACACACGGGCCCAACTGAT
CCATTCTCAAATTATGCTCGGAGTTGGTAGATTGGGCAGAAATTATTACC
CCCATTATATAGAGAGGGGACCCCAAAGCTCAAGGAGGTAAAGCAAACAT
TAGAAACATGATTCCTGTGACTGGCTGTGGAGACTCCGTGTAATAATCAT
TCTGGTATTGCCAATTGCAAAAGGACTGTAAAACTAATAGAGATGCTGTC
TAAATACCGACATCCAATCCTTTCATGCTCTTCTGAGCAGGCTTGAATTT
TCCAGTGCCTCTCTTAAAATACAGCCCGCAGGACTGAGCATAAAACTGCA
TGTGGCCTGACCTGCCAGCACGCGCTGGGAGTATTGCTTCCCTCCTTCCA
CACTCTGCTGTGCTATTAATAGAGCCTCAGATTGCACTAGGGTTCTCAAC
AGCCTCCTCGCGCTGTAGGTTCACATCAAGCTGGTGATCAGCTAACCCCC
CGGGTCTTTCTCATGCAATGCCTTCTGTCAGGCCAGGCCTCCCCAGCAGC
CACTTGGGCAATGAATGTTTTTGAATGCTCATGATTATTTGTGACTTAGA
CTAGAAGGTACAACACCAGATCATGCCCTTCTTTCTCATGTGGCATTTTG
CTGAGTGGGTGATGGGGTCTTAGGGGTCCTGTTCAGGACCCAGAGCTGTG
GCCAGCCACTGGGGGCCACCAGCATCATCGGCCCCACAAAAGTAAGAGGA
TGATGGCGAAATTAATTTTCTGGCTCACTTCAAATTATTCTTATGTTCAT
CCCTACTCCTCTTGGATTAGGTCCTTTCTTGGTCTTGTTCTAATGCTGGG
TGTTTGTATGCACAGCCTCTTTTCCCTCCCACCCATGGACACAGCCACAT
TTTGATTTCTCATGCCCTTTCCAAATAGTAGTAGTATTTTTAGCATGAAT
ATCTTGCTCAGAAATTGGCTGTACAGTCTATCCCCTATTGTCTTCACATC
TAATACTTTTCTTTTTTCCCTCTGCCTCTCCTATGCATCAATACCACGTT
TGGCTAAAGAGATTTGATTTTGACCTATTTGAATTCTCCCTTCACAGATG
ACAGCCCTTTCTCCCTCTCCTTCCTTCCCTTCAATTAGTTTTTTCAGCCA
CTTGGAATTAGCTGATGGGTGATTGTAGAAATTGCAACGTGGGCTACTGT
GGTGGGTCTCAATGTCAACTCCAGGAAACCTCTGAATCTGGGGGGCTCTG
GTTTCAGAATCTGATAGCCAGGCCCTGAACTCTGGAATGTGGGGCTGTGA
CATGAGATACAGCTTCTCTTTGCTCGGCCACTAGAGGGAACTGAAATGTG
GGGCGCAAGAAGGCGTTTCCTCTGTTCGCCAGACGAGGGCGCTCATACCC
ATGGTCCTCCAAAGTTGGAATTTCTCGCCCCAAAACAGATATTGTCGGGT
TGGCCTCCTTGTAACCCAAACATACGATGGGATGACATTACAGCTGTGCT
ACTGATTGCTGCTTTGACCGCCTCCTATGCTGTGTAAATGGCCAAAAGCA
AAGAATTATTAAAAAGCAGGCCCAATGTTGTCCAAGCTCACGTGTGGTTT
GTGGGTCTATGTGTTTGCTGCTGGCAAATTTGCAAGCAGATGGGACTCCA
AGGCAAGGCGTGGAAGTGATGATGGGAACGTTGGAAGTTCACAGACATAA
CTTGTAGAGTGTGTGAGGCCGGGTGCGCGGACCCTGTGTATCTGCAGCTG
CGATACTTAGATTTCAGTTTGGCAAGGCAGGTCACGGTGGAGATGGGGCA
AGCTGCAAGGGTGGTGGAGAGGAGGAAGGGAAGGTGACAGTGGCCCTCTG
TCAACTGTTTCCAGGTGGAGTTGAAAGGTGTAATCATTTTCTTCTGGGGG
CCTTGGCACCTTTCATCAAGACGAAGTTGGTGACTGGTTTAAAAAGATTT
AAAAAATTAAGCTCGAGAGGCCAAAGGAGAAAATGGTTTCCAGGTGGAAA
GGGCTTGACAGAATGGTGCTCTTGTGCCGTGACTCCGAACTCCGTGGAGC
ATTCCAGTGGCCCACTGTACTCCCACCCCTCCAGGCAGCACTGGGAGGCA
GCCAAGTCTAGGAGGCAAAGGGCTCCCTAACTGCCAAGCAGTGAAGATGT
TGAATAAAATATTTACTTACACGTTTAAGAATAATGATGACAGCATGACA
AACAGTGGTGAAACAGCTTTAGGGGACATGGAAGGGCAGCCCTGGGATAT
TTTTAATGAGAACAGTGACTTCCTGTTTAATTCCCGAGGCTTGTCTCTTT
TGCCTACCATACCCACACTGGTATCACAAGATACCGCCCATGATTGGGGA
GGGGGTTCACCAGACTGGCCTAGGGAGTCCCCTGCAGGAAGCTGCCACAT
GGAGAGGCTACAGCCAGCCTCACTCCCAACCCTAAGCTATTGCCCACCTT
TTGCAACTCCTGAAGATTACAGCTTTCTGATCCCTTCCCCCCCCCTACAC
CAGAAGGGTCCTCTGTTGTGGTCATTCAATAAATGATATTTCTTAATTAG
GAATCTAGCTCTTTCTTATTCAGCTGGACTAATAAGCACCCTATGCCCTG
CTTGGGTGTGATAATTTTGAGTTGGAGACAAGGAAAAAGGAGTGAATGAA
AGGGAGTAAAAGTCTTCACCCACAGCACTAGATTTCAGCTATGCCCAACG
TGAAAATGGAAAGGGAAAATGGAAAAAAAAAAAATTGGCCAAACACGCTT
TAGGTTTGTTTTTCCCTCCTTTTGGGAGCTTTTTGCATTTTCCTCCCCAA
TTTGGAAAAAAAATGAAAGAAAACAAATTTCTCTATCATTTAAATAAAAC
AGACCTTTATGTCTCTAAATATAATACATCAAACAATGTTAGGAGTAACT
AAATTATACATAAAGATACTTGTTTGTTAGATTGTTAAAGGCTGTTTGAA
AAATAGAATTTCGCTGGTGAGGTGCCTCACACCTGTAACTCCAGCACTTT
GGGAGGCTGAGGCAGGTGGGTCACCTGAGGTCAGGAGTTTGAGACCAGCC
TGACCAATATGGTGAAACCCCATCTTTACTAAAAATACAAACATTAGGTG
GGCTGTGATGGCACATGCCTATAATCTCAGCTACTCAGGAGGCTGAGACA
GGAGAATTGCTTGAACCGGGGATGTGGAGGTTGCAGTGAGCTGAGATTGA
GCCACTGCACTGCAGCCTGGGCGACAGAGGGAGACCCTGTCTCAAAAACA
ACAACAAAAAATAAGAATATAATTTCACTTTTTGTCAGCCTCACATCCTC
CATGGTTTTGTGTGTTTATTTTTCCAGATATTTTATACCTCCAGTTATGA
CTCTGTAGAAAGATACCATCTGGGGGCCAGGCATGATGGCTCACCCCTGT
AGTCTCAGCACTTTGGGAGGCTGAGGCAGGTGGATTGCCTGAGGTCAGCA
GTTCAAAACCAGCCTGGCCAACATGGCGAAACCCTGTGTCTACTAAAAAT
ACAAAAATTAACTGGGCATGGTGGCAGGCGCCTGTAATCTCAGTCACTCG
AGAGGCTGAGGCAGGAAAATTGCTTGAACCCAGGAGGCAGAGGTTACAGT
GAGCCGAGATCGCGCCATTGCACTCCAGCTGGGAGACAGAGTGAGACTCT
GTCTCAAAAAAATAAATAAAGATACCATCTTGGCTTTCCCATATTATACA
GATCCAGAAGAAAGACCAACTTAGGATCTCTATGCACATGATTATTTCAT
ATTTTTTGGAAGAAAATAAACTAGTGTTGAATTTAAGAACATGCTCAGAA
GTCATGATTTTTGAGGAAGGAGGCTATTTATTTAAATCGATATAAAGGAC
CATTAGTTTTAGACCTGTAATTTATACTTGGTGAAATTTTCATGGAAAAA
AAACAACAACAAAAAAACTCATTTCCCTAAATATTTTCTAGTAAAAACAT
GGCTTGCTTTTTTGGTGCAAAGTCTGCCACGCTGTTTTTAAAAGCGAGGC
TTACGAGACCGTGGGAGAGAGATAAGTGAACAGCCTCTTTAATAAGAGAG
GCGTCCAGCGTGGCGGCGGAATGCAATACCAAAAAGTAAACAAAGAGCAT
CGTGTGAAAAAGAGCAAGTTGAAATGAATCTTGCTTTTCCTATTTGAAAA
ACACGCTCATGGTTGTTAGCAACTGAGTCAAGACATTTAAATCATATATA
TACTTTTAGATCTTGACAGTGACCTTTTATAAGTGTACAGTGGGGATAAG
AAGATGAGCAAAGCCTTGCTGCAGAAAAAGCATTTCAGTTAATTGAACAT
GAAATGTGTTACCATCTGATAACATTAATAATATGTGATCGCTACTTTGT
ATCTAATATGCAGTTCATTTGGTTGGAATCTAAAGCATTCTATAAATGTT
AGAGTATGAATCCTGTTGCAAACCTATAAACTAAGCAGCTCTATTTTGGT
GCATTTTGAAGTATCTCTGTGTTAGTTATCTATGCTGTGTAACAAATTAT
CCCAAAACTTAGCAGCTTCGAACAACAAATATTTATTATCTCAGCATCAA
TCAGGAATGGCTAAGCTGGGAGGTTCTAGTTCAAAGGCTCTCATTAAGTT
GTAGTCAAGGCATTGGCCAGATTAACAATCATTTGAAGACCTGATGAGGG
CCGGCAGATCCACTCATAAGGTGTCTAACTCACAATCCCAGCAAGTTAGT
TTGAGATGTTGACAGTAAACCTCAATTCTTTTCTACACTGGTCTCTCCGT
AGGGCATGGAGAGATGCCTGAGCATCCTCATGACATGGCAGCTGGCTTCC
CCCAGAGCCAATGATCCATGAGACAAAGCAAAACGGAAGCTACAATATCT
TTGGATGATCTAGCCTTAGAATTCACCCATCATCACTTCCTTCAGGTCCT
GCTCCTTAGAAGCCGGTTACTAAGCACAGTCCACACTCGAGGAGAGGGCA
ATTCGACTCCATCTTTTGAAGGAGTCTTAAAGAATTTGTGAGCATATTTT
AAAGGCACCGCAATCCCCTATTTACATAAGGACAGTTGAAAATGATGGTG
GCTTACCTGCTCAAGGTCAACGAACTACTGGTAAGACCCCACCTGGAAGG
CGGCAGGCTTTTTTATTTATTGTAAAGCAAAACAGAAAACCCACATTCTT
GAAATAACTGCACATGAATCCCAAATCTGTCTCTTTCAAATGTCCAAGAC
CTTCTAAAAGTGGCAGGATGCTTTCTGTTTAGAAATGGATGAGATGGACA
CTAGACTGGAAGGGTCAGCCTTTGATTAAGAGTCAGCTTTCCTCTTAATC
AGCTCTGGGACCATGAGAACAAAAACACTTTTCTAAGGGATGTTTTCCTC
CTTTGCAAAATATGATGGGCTAGCCGAATGGTTTCCAAAGTTGGTGGCCT
TTAAGTCCTCTGGGGACTTAAAAACTCACTGATCTTGTGTTAAATCCACA
ATGTCCAGGAATCTGTACCACTTAAAAGCACTTGGGGACTCTGGCGGCCT
GTTTTGCAGACAGTAGGAACTGCTCGGCTACATGATTTCTCACTCTTCCA
CTTTTAACATTATTTTATTTATTTTTGAGACAGAGTCTCTCTTTGTCACC
CAGGCTGGAGTGCAGTGGCATGATTTTGGCAACCTCCGCCTCCTGAGTTC
AAGCGATTCTCCTGCCTCAGCCTCCAAAGTAGCTGGGATTACAGGCTCCC
GCCACCATGACTGGCTTTTTTTTTTGTATTTTAGTGGAGATGGGGGTTTT
ACCATGTTGGCCAGGTTGGTCTGGAACTCCTGACCTCATGTGATCTGCTC
ACCTCTGCCTCCCAAAGTGCTGGGATTACAGGCGTGAACCACCATGCCTG
GCCAACATTATTTTAACTCTCCCCATCAGACTGGGTATGCCCATGTAAAT
TGTTGGTTCTCCATCTTCACTACAATCATGAGCAGAATTTTTAAAAAATA
TGATACCTAGGGCCCTCCCTAGGCAAAATATAAGTCATTCTGGGGTGGAA
CTCTGGTACCATCACGGGTTGTTGGCTTGTTTTCATCAGTACATTTAAAA
CTAATCATGTTTAGCCTTGTTGGCACATTAGAATCACTTGGGGAGCTTTA
AAAAAGCCCACTGCCCAGCCTGTCCCCCAGGCCAATTAAATCACAATCTC
GTGGGAAAACCAAGAATCAGCATTTTTTAAAGTTCCCCAAGTGATTACAA
CATACAGCCAAACTGACCTATGTTTGCCACATTTGAGATAATTCTAATGC
TAATTCACCTATAAGGGATTATTCAGAAAAAAATCCCAACATTTAGATGC
CACAGTACTCTAAGAAAAAAAATGCATTTAAAGTGGAAGATATTACAATT
TTGAAATGAAAGATATTAAAAATTAAATGGAACTAAGTTCCATTTCTGGC
AATATGGTAGACTAAGTAACTTGAAAATCCTCCCATCATAAACCACCTAT
AAATACTGGTCAGAATGTAATAAACACCCATTTAAATGAGCTCTCAGGAC
AGTAAGCAAAGGCTCTCAGAGTCAGGAAGAAGAGGGAGATTCTAGCATGG
TATGCAAGTAAGCTGAGGTTGAGCTGGTCTTAGGCAGGTTTGCTGGTGTT
GGGAACCTGAGGTTTGAGCATCAAAATAGGAAGGAGACTATGCTTAAGGT
CCATTAAAAGTGGGAAAATGGAATTCAGAATTCCCATAAAGCTGGAATCC
CATCAAGCTAGAACCTCCTGAATCACTAGAGAAATAATCACTGGAAAAAT
AATCTCCCCAATGTCACAAGGAAACAAGAAAATGTGCCTGTCTTTGCAGG
GGTTGAGGGTGGGGAATAAAGGGCTTTACTGAGAATTTGAGATTATAATG
TGGTATGGTCCAGGAACCCCAAAGCTGAGAATGAATACAGAAATACAGAC
CCAATGCCAAACTATACAATGTATGTGGATATAATCCTCCACAAGCAAGA
TGTAGCAGACACAAAGGTCCCAAGAACCTCAGGTAACAGAACTATCAGGC
AGACTATAAAATAAGCAAATTGAAAATTATTAAAGACACAAAGAGGCCGG
GCGCGGTGGCTCACGCTTGTAATCCCAGCACTTTGGGAGGCCGAGGCGGG
CGGATCACGAGGTCAGGAGATCGAGACCATCCTGGCTAACACGGTGAAAC
CCCATCTCTACTAAAAATACAAAAAAATTAGCCGGGCGTGATGGCGGGCG
CCTGTAGTCCCAGCTACTCGGGAGGCTGAGGCAGGAGAATGGCGTGAACC
CGGGAGGCGGAGCTTGCAGTGAGCCGAGATTGCGCCGCTGCACTCCCGCC
TGGGCCACAGAGCGAGACTCCGTCTCAATTAAAAAAAAAAAAAAAAGACA
CAAAGGAACCTTTGACACCATGAGAAAATAACATAACACTCTAAAAAAGG
TAGATTTTAAATAGAACTAAATAGAATTTCTAGAAATGACAAATATAGTC
ACCAAAATGAAAAGCTCAGTGATGAGTTAAACAGCAGATTAGACAGAGTC
GAAGAAAGAACCACAGATGGATCTGAGAAAATTGCCCAGGAAGCAGCAAA
GACAAAGTGAAGGAAAGTCTGACAGATTCAGAGTGTGCTGGATAGAAGGA
GAAGATGCATTATACATTTCATATAAGTACCAAAAGACAATGAGAGGGAT
ACTTCATTTAGAGAATCCCAAGATTATGCATATACAATGAGTATTGAATC
AGATAAAGAAGAAGAAATTCATACTTGAATATAGCAGAGTAAAAATGTAG
GAAGCCAAAGACAAGGAAAAAGTCTTAAAACCAGAGAGAAAAGACAGATT
ACCTACAAAGGAATGACAATTAGACTCATAGCAAATGTTTCAGAAATAAA
GAATAGGAAGACATGGTATATTCAAAAAAGTGCTGGGGTAAAATAACTGC
CAATCTTGAGTATTACACCCAGAGAAATCATCATTCAGGAATGAGAGTGA
AATATGACATGTTTGTCTTAGCGGAGAGAGCGTACCACTCAACAATCCCC
TGAAAAAAACTAAAGGTATGTTTCAGGGGAAAGGGTCTATATCTAGAAGG
AAATTGGTAAATAAGGGCAAATCTAAACGATGAATTGACTGTATATAAAA
TTACAATAGAGATTAAAATTAGGGGTATAAAAAGTAGGTGGATCTAAAAA
TAAGCAACAGTAAAACATAATGAGAGGATGTAACTGAAGTTGAATCATTC
TTAGCTTATTGGATAGTTCTAGGGCATTTGATTTACTTTAGATCACATGT
ACAGGTTAAAATTGTAATCACCGAAAGAGTAGAAATAGAATTTACAACTT
CCGGCCAGACACAGTGGCTCACGCCTGTAATCCTAGCACTTTGGGAGGCC
AAGGCAGGCAGATCAATTGAGGTCAGGAGTTCAAGACCAGCTGGCCAACA
TGGTGAAACCCCGTCTCTACTAAAAATACAAAAATTAGCTGGGTGTGGTG
GTGGGTGCCTGTAATACCAGCTACTCGGAGGCCGAGTCAGGAGAATCGCT
TGAACCCAAGAGGCAGAGGTTGTAGTGAGCTGATTGTACCACTGCACTCC
AGCATGGCTGACAGAGTGAGACTCTGTCTCAAAAAAAAAAAAAAGGCCTC
GGCCTCCCAAAGTGCTGGGATTACAGGTGTGAGCCACCATGCCCGGCCAA
TAGCATCTCTTATACATTGCTAGTAGGAGTACAAATTGGAACACCACTTT
GGAAAACAGCTTAGTATTACCTTGTAAAATTTTACATTCACGTATGTTAC
GACCCAGCAATTACTCCAAAGAGAAATTCTGATCTATGTGCATCAGAAGG
TAAAAATGTTCATAATAACACTGTTTATAATAGCCAAAAAAAAAAAAAAA
TTCCTGAAAGCAACCCAAAGGCTTGTTTGTGAGAATAGAGAAACTAAACT
GTGGCACAGTCACATAATGGAATATTATACAACTGGGAGAAAGAATGAAC
TACAACCTGATACAAAAATCTAATTTTGTTCCCCCCACCCCCCCAGGGCC
CTGGCTAGAGGATCTAATTGATTCTTAATAATTTCATATTGAGTAAATTA
TCAAGCCTCAGAAATTTTTCATAAAGTTTCAAAACAAAAAGTAAAACAAA
ACAAATATTAGTCATTGATATGCATGATAAAACTTTTTAAAAAGCGAAAA
ATCATAACAAATACAAGATTCAGACTGGTGGTTACTTTAGAGGAACAAAA
TAGGGAGGAACACATAAGCAGATGTTACATAAGTCAAGTCATTGTTTCTG
TTTTAGTTCTCTGGTTGAATAGCAGGTTTACAGGTATTCATGATATCAGC
AAATAAAATAAAATAGGGCCATGAGTATACACAATGATGATAGTGTGTTA
TACTAGAGATTGTGACTAATATATTTTTGTGCCCCTAAATTGTGATATTT
TGTGATCTTTAAAAATATCAGCAATCACAATAAATGTAAATGGACTAAAC
TTACTAGTTAGCACAATAATGAACCAGAGCTAACTATATGCTATTTACAG
GAGACTGAACAAAAAATTTGGGACATGGAAAGGTTCAAAGTAAAAGGAAA
GAGGGAAAGAGAAAGATCTATAAGTGAAGGGCTAATTAAAAGAGTAGTTA
CATTTATTACAGACAAAGTAGACTTTAAAGCAAAAGGCATTAGGGATAAG
TATCTTCTGTGCCAATATTAAAGGAATAATTCCTAGGAAAATAGCAAATC
TAAGCTCGTATGCACTGAATAACAAGGCCTTAAAATACACAAAGCAAAAA
GTATGAGAAGCAGAAAATGTATAGTTACAGTGGGAAATTTTAACAGTTCT
TTTTGATGCACCTGACAACATACTTTTAAAATACAGCAAAATTTGAGAGA
ATTACAGGAGAAACTGGCAAATCTAGTCATATAGAAATTTTAATGTACTT
CTATAAAAACCAACAGGTCAAGCTGACAAAATATTAGTAAGGCTACAGGA
AATCTAGACAATATAATTAATAAGTCTGATCTAATAGACCCATATAAAAT
GCTGTACTTAAAAATTAAGGCATATACATTCTTTTCAAATCTACATGAAA
TATTTATAAAAATTGACTGCACCAAAGCAAGTTTCAATAAATAACAAAGA
ATCTGCTTCATATAGATCACATTCTTTGACCTTAATTCAGTTAAGTTAGA
CGTTAGTAACTAAAGAATAACCTCAAATACCCCTCAAATACACCTGTATT
AATTCACTAGTCAAACAAGAAATCGTATCTGAAATCTAATTTTTTTTTTT
TTTTAAACAGGATCTCGCTCTGTCACCCAGGCTGGAGTAGTGACATGATC
ATGGCTCGCTACAGTCTCAACTTCCCAGGCTCAAGTGATCCTCCCACCTC
AGTCTCCTGAGTAGCTGGGCCTATAGGTGCATGTTAGCACACCTGGCTAG
TTTCTAGAAAAGTTTTCTTTTGCAGAGATGGGGCCTCACTATGTTGCCCA
GGCTGGTCTCAAACTCCTGGGCTCAAGTGATCCTCCCACCTTGGCCTCCC
AAAGTGCTGGGATTACAGGCATAAGCCATTGTGTCCAGCCTAAAATCTAA
ACTTTTTTTACCTCATTGGTGATAAAAAGTCTATATATCTTGTGGAATAT
AGCTTGAGTGGTACTTAAAGTGAAATCTGTAGTCTTAAATGTTAAAATCA
GAAAACAGATGGCTAAAAATTAATTACCTAAGGCAACAATTCAAAAAAAA
AAAAAAAGAGCAACAGAATAAATCCAAAAAGAGCAGAAGCAGAAGGGAAT
ACTATATGATATATATTAATATTACATTACATATTATATTCTCACAAACT
ATATAATAGTATATATTTTCTATTGTGCCATATATAACAATATAATATAT
AATAATATATAAGAACAAAACTTACTGGAACAGAAAAAGAAACAATATTG
AAACTAAAGTCTGGTTCTTTGGAAACACTAATAGAAAGTAGAAAGAAAAC
ATTAGTAAAAAAAAAAGATCTCTGGCACAGCTGATCCAGATAAGAAAAAA
TACATATAAAATGATATTTAGAATGAAAAAAAAACATTATGGATAGATAG
AGCAGAGATTAAAATACATTAAGAGATAACTATGAACAGCATTTTGTCAA
CACATTTGAAAACCTAGATGGTATGGATAATTTCCTTGAAAAACATAGTG
TATAAAAATAGATTCAAGAAGAAAACAGAAAACCTGAAGAGATCTACGAG
CATTAAAGAAATGGAATCAGTAGTTAAAAATCAACCCACGAAATGTCTAT
CCCAGACCCAGACAATTTACCTGCAAAACTACCAAACATTCAAGGAACAC
ATAATTCCAATCTTACACACACTGTTCCAGAAAATGAAAAAAGAAGGAAC
ATTTACCGGTTCATTTTATGAGACCAGTATAACCAGACAAAGGCAGTAAG
AAAAGGAAAAACTGCAGCCAATTTGACTTATGAACATGAATACAAAAAGA
ATCCTAGAATGAAATTAAATGTTGGCCATCATTCATTCATTCATCCACTC
ACTCATTCAGTCAATCATTATTTATTGAGCGTCAACCGTGCGCCAGCAGG
CACTGTGCTAGTACATGGAGAGCAGAAAGGCACGGGAGCTTCTGGCTTAG
AGGAGATGGGCAATAAAGCAAATGATCATACAGGGTAAGGTACACAGAGG
ACGTTCTGGTAAGGTAACTGCATATCAAAGGGCATTCGACCCTGTCAGAG
AGGTCTGGGAAAGATTTCCAGGCATGTAAGTGGAGTAAGGGTGTATGTGG
GAAGACTGTTTTGTAAGCTGTTGCAGGGCCTCAGGTGGGAGATCTGGGAT
GCAGCAGCAAGAAAGATGGATTTGAACTTGGGCTTCCTTTAGAAAGGCTA
AGTGGAGATGTTGAATAGGAAATTGACCAGAGCCTGGAGCTCTTCAGGAA
GGGTGGGGCTGGAGATTTCAATTTGAGTGGCATCACCATGTGTTTAAACC
CATCCTGGAAGATTGAGTTTGAAGAAGGAAGTGTCCAACATTGTCTTGGG
CTGTTGAGACTTTCAGAGGGTTGAGGACTGATATTGTGCTGCTTGAATTC
TCCTGATGCAGGGGCTACATTGAGTGAGCTGGAGAAAAAAAATGCATAAA
ATAATAATAATAATAATAATAATAATAATAATAAGCTATTACAAATAATG
TAGAGCAAAGGGGCAGCAAGAGGGAATTTTTTTGGACAATGAAACTGTTC
TGCATCTTGATTATGGTGGTGGTTACATGACTCTATACATATGTCAAAAC
TCATAGGATTACAGACCAAAAAGAGTAAATATTACCGTATATAAATTAAA
AATAAATGAGTAAAAACAATGTAGTAATGGAGACTTAAAATCCAGTTCTT
TCTAAGCCCTGACTTTGTAACCGCAGCTCTAGCCCCTCTCTGGATTTTAA
ATCAGTTCTATAAGTGTCAGCTTGTGGAGGTCTATACCAGACAGGAAGGG
CCCCCAACTCTCGCCTTGTGAGGGACAGAATAAACACGCAGGCAGCAGAG
GCCACACGGCATTGGACTGATGGTCAGAGGGTGGGGGTGGGGTGTAGCCT
GGTGAGTTTGGCACCTCTGAGACGCTGATGTATAATGAGGGGATTAGATT
AGGAAAGGCCTTTCTACCTAGGATGGCCTGTGGTTCTACTGTAAAAATCC
CAAACACAATACAATTAGCTCTGTTGTCTGCATTTTGTTTAGAATAATCA
ATCATAATAAACAATCATTGTAACAACTGGCTGTTCAACACATGAGACCC
CAGATGATTTGGGAAGGAGCTTGGAGTGACAGGAAATGTTTGGGTTTGTG
GTTTAAAGCCTTAGAGCACCTTCTCAATATGATTATATTGAGTAGTGATT
GATAATAAACACGACTCAGGTTTACAGTGAAAAAGGAACTTTTACAACAT
TGGTTCACTTCAGCCTCTCACCTTCACCACATCAATCCTGTCAAGGAGGA
ATTACTGCAATTTAGGGAACAGGGAGACTGAGGGTCTGGTCACTCAAGGC
TATGGCTGGTGTTGAGATTTTCCCAATATTCCATTTTTCCAAAGCCCACA
GTGGATTTGGTTCAGTTTTGGTGTTGAGTGTATTCCTTTGTCTCCTAATC
CTATGAAAATTAATGGAAAAGTGTTAATTGGGCATCAATTCATGCTTAAC
ATTAATCTCAGTATTTGATGAACCACAACTTTATGTTGCCCCTCATGCCA
TATTAACTCAGTTTATTGCAACAATTTAAAACGATACAGATTTAAAACAA
TATGGGTAATTGTATCCGTATTGTTTCAAATGCCCCATAAATTGAAACCA
GCCCGAATTTGGGCAGTCTGGAATCTGCCGGAGAAACTTTCATGCGATGC
CTTTGGAAGGCTACAGACATTGTCTTTTTGGAGTTTTCAGTGCATGAAGG
TATGAAACCGCATTTATTAAGCACCTACTGTATGCCAGAACCCGTGCTGC
ACAATACTACTGCTGCTAAGGTGGGAGTGATTCTGAAGCCTTCTGCCACC
CTAGCTACCTCTGCAGGTCGTGAGGGGTCTTGGGCTATTTCAGTATCATG
CACTTTACTATCCTGGCATACAAAGGCTGGGTGAGAAATAAAATATATAA
CGAACGGATTACACAGGGGTTTCCTGAAATAACCACCCTTCCCATCCATC
CCAGAGACACCCCAAAAGTACTTCTCGTTATATACAAACATTTGCTTTGA
ACCTCAATCATGTGACCTTGACTCCTATAACCTATCTTATTACATTTTTA
AAACACTGTATGATTAACGCGGAAACCCTTTCTTCGGCACTTTCTCGCCA
CTGGAATCGCGTCAGTTTCTCAAAGTTCCAAAATAACCTTTCCCGGGCAC
GGATTGGTACCTCTACTGGGGAAGGGCGGGGAACCGCGCAAGACGTGCCG
GTGTGGAGCCAGAGCCAGAGAGAACTTCCAGCGCAAAAGGAAAATAAAAC
TTGTGGCTGGTGTTTGTGCAGGAGGGTCTCCGCCATCCTGAAGCCCCCCG
ATCCTGGGGCGTCTCGGGGGCCGCCAAAGGAGCGCCAGGGTGTGGGTTTG
CTCCCGACGTCCTTGACCTAAATTTCTGAGCGGTGGCTGGAAACAGGGCA
CAGCGGAGGGCGGGCGGCTGGTGCCATTCCCGGATCTCGGCGGCAGGGGC
CGGCAAACTTGAATGGAGAGGGCGAACTAGAGAGGGTGGGGGGCGTCTTC
TCCCAGGTCCGGGTGAGGAGCCGCAGCAAGCTCCCCGCGCCTCCCCTCCC
CCGATCCACCCGCCCCCCGCAGCCCATGTGATCCAGGGAAGTCGGGGTGC
GCTCCCCCTCGCCCTGCGCCCTGCCGGCCCGGAGGCGGGGTCCCCTCCGC
CCGCGGGGTTCGCGCGCCACCCTTGTGGGTCCGGCCGTGGGGGGCCGAGT
GTGCGCGCGCGGGCAGGCGGGGGCCGCACGGGGGTGCGTGACGTCACCGG
CATTGGTTACACGACGTTCTAGAACTCCGCCCCACGTGCGCCGGGGAGGA
GGGGGAGGAGGAGGAGGAGATGGGGGTGGGGAGGAGGAGGGGGAGAGGTG
GGGATGGGCCGGGGGGGCGGGGACGGGGGGGTGTGCGAGGCAGCGGGGCT
GAGCTAAGCCGAGCCCACGTGTGACGGCTCTCGCCGCTGCCCCGGCTCCG
CCGCTCGCAGAGAGATTCGGAGGAGCCCGGGCGGGGGGGAGGAGGAGGGG
GAGGAGGGAGCGGAGATCTCGGGGCTCGGAGCCGGCCGCCGCTCCGCTCC
GATCGCTGTGGGGCTTGGTTTTTTGGGGGTGGGGGGGCGGGGGGGCTCAG
ATATG

JAK2

The JAK2 gene is located on Chromosome 9. JAK2 protein promotes the growth and division (proliferation) of cells and is part of the JAK/STAT signaling pathway important in transmitting signals from the cell surface to the nuclei. JAK2 is especially important for controlling the production of blood cells from hematopoietic stem cells. These stem cells are located within the bone marrow and have the potential to develop into red blood cells, white blood cells, and platelets. Essential thrombocythemia is characterized by an increased number of platelets, with the most common mutation being V617F found in approximately half of the affected people, with a small proportion having a mutation in exon 12. The V617F JAK2 gene mutation results constitutively activated JAK2 leading to the overproduction of megakaryocytes, and hence excess platelets. As a result, there is increased risk of blood clots and decreased availability of oxygen. Overproduction is also associated with primary myelofibrosis, as megakaryocytes stimulate other cells to secrete collagen thereby replacing bone marrow by scar tissue. The V617F mutation is found in approximately half of individuals with primary myelofibrosis. A small number of people with this condition have mutations in the exon 12 region of the gene. These JAK2 gene mutations result in a constitutively active JAK2 protein, which leads to the overproduction of abnormal megakaryocytes. These megakaryocytes stimulate other cells to release collagen, a protein that normally provides structural support for the cells in the bone marrow but causes scar tissue formation in primary myelofibrosis. The V617F mutation is occasionally associated with leukemia, other bone marrow disorders and Budd-Chiari syndrome.

Protein: JAK2 Gene: JAK2 (Homo sapiens, chromosome 9, 4985245-5129948 [NCBI Reference Sequence: NC_{—}000009.12]; start site location: 57256743; strand: positive)

Gene Identification

| | |
|---|---|
| GeneID | 3717 |
| HGNC | 6192 |
| HPRD | 00993 |
| MIM | 147796 |

Targeted Sequences

| | | | Relative |
|---|---|---|---|
| | | | upstream |
| | | | location to |
| Sequence | Design | | gene start |
| ID | ID | Sequence (5′-3′) | site |
| 12063 | | CGCACCAGTTTGTCCACGTCCAG | 1663 |
| | | TG | |
| 12098 | | GCCGTCACTGCCGACATAAGCACA | 1811 |
| | | GAC | |

Target Shift Sequences

| | | Relative |
|---|---|---|
| | | upstream |
| | | location |
| | | to gene |
| Sequence ID | Sequence (5′-3′) | start site |
| 12063 | CGCACCAGTTTGTCCACGTCCAGTG | 1663 |
| 12064 | GCACCAGTTTGTCCACGTCC | 1664 |
| 12065 | CACCAGTTTGTCCACGTCCA | 1665 |
| 12066 | ACCAGTTTGTCCACGTCCAG | 1666 |
| 12067 | CCAGTTTGTCCACGTCCAGT | 1667 |
| 12068 | CAGTTTGTCCACGTCCAGTG | 1668 |
| 12069 | AGTTTGTCCACGTCCAGTGT | 1669 |
| 12070 | GTTTGTCCACGTCCAGTGTC | 1670 |
| 12071 | TTTGTCCACGTCCAGTGTCA | 1671 |
| 12072 | TTGTCCACGTCCAGTGTCAA | 1672 |
| 12073 | TGTCCACGTCCAGTGTCAAC | 1673 |
| 12074 | GTCCACGTCCAGTGTCAACT | 1674 |
| 12075 | TCCACGTCCAGTGTCAACTG | 1675 |
| 12076 | CCACGTCCAGTGTCAACTGA | 1676 |
| 12077 | CACGTCCAGTGTCAACTGAG | 1677 |
| 12078 | ACGTCCAGTGTCAACTGAGC | 1678 |
| 12079 | CGTCCAGTGTCAACTGAGCA | 1679 |
| 12080 | TCGCACCAGTTTGTCCACGT | 1662 |
| 12081 | ATCGCACCAGTTTGTCCACG | 1661 |
| 12082 | GATCGCACCAGTTTGTCCAC | 1660 |
| 12083 | GGATCGCACCAGTTTGTCCA | 1659 |
| 12084 | GGGATCGCACCAGTTTGTCC | 1658 |
| 12085 | TGGGATCGCACCAGTTTGTC | 1657 |
| 12086 | TTGGGATCGCACCAGTTTGT | 1656 |
| 12087 | CTTGGGATCGCACCAGTTTG | 1655 |
| 12088 | CCTTGGGATCGCACCAGTTT | 1654 |
| 12089 | GCCTTGGGATCGCACCAGTT | 1653 |
| 12090 | GGCCTTGGGATCGCACCAGT | 1652 |
| 12091 | GGGCCTTGGGATCGCACCAG | 1651 |
| 12092 | GGGGCCTTGGGATCGCACCA | 1650 |
| 12093 | GGGGGCCTTGGGATCGCACC | 1649 |
| 12094 | TGGGGGCCTTGGGATCGCAC | 1648 |
| 12095 | CTGGGGGCCTTGGGATCGCA | 1647 |
| 12096 | TCTGGGGGCCTTGGGATCGC | 1646 |
| 12097 | ATCTGGGGGCCTTGGGATCG | 1645 |
| 12098 | GCCGTCACTGCCGACATAAGCACAGAC | 1811 |
| 12099 | CCGTCACTGCCGACATAAGC | 1812 |
| 12100 | CGTCACTGCCGACATAAGCA | 1813 |
| 12101 | GTCACTGCCGACATAAGCAC | 1814 |
| 12102 | TCACTGCCGACATAAGCACA | 1815 |
| 12103 | CACTGCCGACATAAGCACAG | 1816 |
| 12104 | ACTGCCGACATAAGCACAGA | 1817 |
| 12105 | CTGCCGACATAAGCACAGAC | 1818 |
| 12106 | TGCCGACATAAGCACAGACA | 1819 |
| 12107 | GCCGACATAAGCACAGACAA | 1820 |
| 12108 | CCGACATAAGCACAGACAAC | 1821 |
| 12109 | CGACATAAGCACAGACAACT | 1822 |
| 12110 | CGCCGTCACTGCCGACATAA | 1810 |
| 12111 | TCGCCGTCACTGCCGACATA | 1809 |
| 12112 | ATCGCCGTCACTGCCGACAT | 1808 |
| 12113 | AATCGCCGTCACTGCCGACA | 1807 |
| 12114 | CAATCGCCGTCACTGCCGAC | 1806 |
| 12115 | CCAATCGCCGTCACTGCCGA | 1805 |
| 12116 | GCCAATCGCCGTCACTGCCG | 1804 |
| 12117 | AGCCAATCGCCGTCACTGCC | 1803 |
| 12118 | CAGCCAATCGCCGTCACTGC | 1802 |
| 12119 | CCAGCCAATCGCCGTCACTG | 1801 |
| 12120 | CCCAGCCAATCGCCGTCACT | 1800 |
| 12121 | ACCCAGCCAATCGCCGTCAC | 1799 |
| 12122 | TACCCAGCCAATCGCCGTCA | 1798 |
| 12123 | CTACCCAGCCAATCGCCGTC | 1797 |
| 12124 | CCTACCCAGCCAATCGCCGT | 1796 |
| 12125 | GCCTACCCAGCCAATCGCCG | 1795 |
| 12126 | TGCCTACCCAGCCAATCGCC | 1794 |
| 12127 | TTGCCTACCCAGCCAATCGC | 1793 |
| 12128 | CTTGCCTACCCAGCCAATCG | 1792 |

Hot Zones (Relative upstream location to gene start site)
1550-1900

### Examples

Genetic Code (5′ Upstream Region)

(SEQ ID NO: 13675)

GTCATTTATTTCTGCTGTGAACTTCATTTTTTCCTTCCTTCTGTTAGCTT
TGGGCTTTGTTCTTCTTTTTCTAGTTCCTTGAGGTGTAATGTAATGTTGT
TTGACATCTTTCTTCCTTTTTGATGTAGGTATTTATTGCTATAAACTTCC
CTCTTATAACTGCTTTTGCTGCATTTAATACTGACTATAATAAGATACGA
TGTAATAGATTTCAAGGAATTATGTATTTTTGAATAAATTAATTCTTTAA
AGTTGCATATCCAGTTGCAGATGAACTTCAAAAATCTTGCAGTTTTATAT
CTGTTACAGTAATTGCCAGGTTTTGTTGTTGTTGTTTTGATACATTAGAA
GTTCTAGAATTGTTATATCCTCTTGATGAATTAATCCCTTTATCATTCTA
GAATTACCTTGTCTCTTTACTGTTTGTGACTTAAAGTCTGTTGTATCTGA
TATACCTTTGCATGGAATATCTTTTTCTATCCCTTTACTTTCAGTCTATG
TGTATCTTTAAAGGTGAGATGAGGTTTTGTAAGTGGCATGTAGTTGGGTC
ATGTTTTTTAGTCCATTTAGCCATTCTCTATCTTTTAAGTGGAAAGTTTA
ATCTATTTACATTCAAGTTTATTCTTGATATGTGAAGGCTTATTCCTGTC
ATTTTATTAATTGATTTCTGGTTGTTCTGTAGGTCCTTTGTTCTTTTCTT
TCTCTCATATTGTTTAGCATTGTGGTTTGTTGGTTTTCTATAGTGATAAC
ATTTGAATCCTTTCTTGTCTGTGTGTGTTTGCTTTACCAGTGGGTTTGAT
ACTTTCGTCATCTGTTTTTCATAATGGTAGTAATTGTCCTTTTTGTTTGT
TTGTTTGTTTCTTTTTTGAGACAGGGTTTTGCTCTTGTTCTGTCCTCCAG
GCTGGAGTGCAGTGGTGTGATCATGGCTCACTGCAGCCTCGACCTCCATG
GTCTCAGGTGATCCTTCTGCCTCAGCCTCTCAGGTAGCTGGGACTACAGA
AACCTGCCACCATGCCTGGCTAATTCTTTTGTATTTTTCGTAGACATGGG
GTTTTGCCATGTTGTCCAGGCTGCTCTTGAACTCCTGGGCTCAAGCAGTC
TGCCTGCCTCAGCCACCCAAAGTGCTAGGATTACAGGCTTGAGCCACTGT
GCCTGGCCTGACATTGTTCTTTGACTTCCATATGTAGAACTCCCTCAAGC
ATTTCTTGTAGGTCTGGTCTAGTAGTGTTGAATTCCTCAGCTTTTGCTTG
CCTCAGAAAAACTATTTTTCCTTTGCTTAATGAAGGATAATTTTGCTGGG
TATAGTATCCTTGACTTGCAGGTTTTTTTCTTTCAGCACTTTTCATATAT
CGTTCCATTCTCTTCCTGGCCTGTAATGATTCTGCTGAGAAATCTGCTGT
TAGTCTGATGGAGCTTCCCTTAGAAGTGACTAGACTCTTTTTTCTTGCTG
TTTTTAGAATTCTCTCTTTGTCTTTGACAAGCTGTTGTCTCTGACAACAG
TTCTCTCTTTGTCTTTGACAAACTGTTGACAGTTTGACTCTAATGTGTTG
TGGAGAACCTGTTGGAATTTTGTCTTTTTGGGGATCTCTGAGCTTCTGTA
TCTGAATGTCTAAATCTCTTGATATACTTGGGTAGTTTTCAGCTATTATT
TCATTAACCAGGTTTTCTATTCCTTTTGTATTTTCATTGTCTTCTAGAAT
ACTGAAAATTCTAATATTAGTTTGCTTTATGGTATCCCATATGTCATGCA
GGCTTTGTTCATTCTTTTTTCTTTATTTTTGTCTAATGGGGTTATTTCAG
AAGACCTGTCTTCAAGTTCAGAAATTCTTTCTTCGTAGATGCTCTAGAAT
GTATTTTTTATTTCATTAAATGAATTCTTCAGTTTCAGGGTTTCTTGTTT
TCTTTTTAAATGATATCTCTCTCTTTGGTAAATTTCTCATTGATATCCTG
AGTTGTTTTTCTGGTTTCTTTGTATTGTTTATCTGTATGCGTTTGTATCT
CCCTGAGCTTCTTTAATATCATTATTTTTAATTCTTTTTCTGGCATTTCA
TGAATTTCTTTTGCATTGGAATCTTTTGGTAGAAAATTATTTTGATCCTT
TGGAGATGTCATATTTCCCTATGTTCCCATGTTTCTTGTGACCTTACTTC
TTTGATATCCACACATCTGGTGTAATCATCACTTCCATTTTTTTGAATTT
GCTTTCATAGGGTAGGACTTTTTCCTGAAGATTTGACTGGGGTGTTTGTT
GGCCAGGGCACTTTGGGTTTGAATCTGGGTGCATGCAGTAGTGTAGTCTC
TGTAAGATTTTTTTTCCTTTGTAAACAGCATCAGTGGTGTCTGTGATTTC
CTCAGTGGCATAGTGTGTGGTTGTGGAGGCTGTGGTGAACTTTTGCTGGG
GATGGTGACACCAGCTGGACTGATCCTCAGTCCTCAGTTGTGGCAGCAGT
TGGACAACCATGCCTGTACATTAGCCCCAGGGTGGCTTACATTAGTAATG
GTGTTAGTGGGTCCAGGCAGTCCAATTTTTGGGTCTCCAGGTGACTTGTT
TGGGTACCAGGAGTGGCAGTGATGGGCTGGGCAGCTGAGTGGGTCCACAG
GCCCCTGGGCAGTGAGCATGGCATGGGTTATGTCAGTAGCAGTGGTAGGA
GAACCTCTGGCTGTCCAGTTGTCTGTGCTTATGTCGGCAGTGACGGCGAT
TGGCTGGGTAGGCAAGTCCTAAAACCTGCAGGTGGCAAGTGTGAGTGGGA
ACCAGCTGTGGTGGTAGTGGCAGGTTGGGTGGGCCACATCCTCAGACCCC
CAGGTGGAATGCTCAGTTGACACTGGACGTGGACAAACTGGTGCGATCCC
AAGGCCCCCAGATAACATGCTTGGATACGTGGGAGTGGGGTGCTGAGCTG
GGCAGGGTGAGAGTATCCTCAGGCCCTCCAGTGGTGTTAGCAGGTGCTGT
TTGTGGTGGGCAGGAGCAGGATGATTTCCAATTTCCTGGTGGAATGTTCA
GGTGGGGGCAGCAGTGGCTGTGCTGTGCCCTGATGCTGGGGAGGGTGCAG
TTGCTGTCAGTGGGAGCAGTTGTAGGGAGTTGGCTAAGGAGTGTGCACTG
CAGCTGCAGGTGGAGGCTGTAGATGTGATGAAGCTGTACTCAGGGTGCAT
GCAAATTTGCATTTTGACACCTAGCGGCAGCAGCCTGCAATGGTGGCAGC
TGTAGGTGGTAGAGCTTGTCCTCAGGGCACATACCAATATATGGCAGCCC
TTCTGCTGGGAGCAGTGGGGTTATTGCCAATGGCTTGTGCTTTGGTCCCA
GAGGCGGCAGCCAGCAATGGAGGTGACTGTCGGTGGAGGATGTCAGTGGG
GCTCTAGGGGTGTGGATATGCAGGGGCTGTTGGGCTCCAGGGTAGGAGGC
ATTCTGGTGTGGGTTGGGCTTTAAAAATGGCACCGTGCTGTAGCTGCTTA
GGACTCAGGGGTGTGTTGGACCAGCATAAGCTCCCTCTCTAAAGCAATGT
CATTGTGCAGTCTCCAGGCAGCTCCCTATGTTACTCCCAGGGCCCATGAA
AGTTGACGGGCTCTCTTGTGTCTGGGATTGCAGGAGTTTGCAGTGAAAAT
GTGGGCCACTGGGAGTCTCTCACTTACTCTTTCCCCACATTGTGCAGGCT
CTCTAGGCTTCTGGCTGATCCTGGCTGAGCAGGCTGCCCCACTTCCCTCT
CCTTCCTTGCATTAGGTGTTTTCTATCACTTCTCTGTTGAATTTCCGTGT
TCTCTCTTAGATGACCTATTCAAAGTGTGATTATCTACTCGCTATTTTGG
TTCTTCTTTGTGGAGCAGGTGAGTACCAGATAACTCTAGTCAACCTTCTG
GACCCCTCTTCCCCCAATTTGAGATCTCTTCTTCTGTTGTCTGTAACTGA
GTTTAATGCTTGTTTGTTCATGTTAGGATTTTATATCATCGTCCTCAATT
AGGTTGTTAACTGGAATTTTATAATCTTTGTCCACAGGAAGTTTAAAATG
TATGATTTCTTGCATTGTGCTTTGTATGTAGTAATACACGATATTTATCC
AGTTAATGGATTTGACAGCCATTGCTGTCAAGGAGCAGTCCTTCTTTGTG
TATGAAGGGTGCCTTATCAATATTATTTCCATTTGTAACTTTATTTATTT
ATGTATTCATTTTTGAGACAGGGTCTTGCTGTGTCACCCAGACTGGAGTG
CGGTGGAGTGCGGAGGTTTGCTGCAGCCTCATCCTCCCAGGTTCAAGCAA
TTCTTCCGCTCCACTCCCAGAGTAGCTAGGACTACAAGTGCGTGCTGCCA
CGCCCAGCTAATTTTTTTCTTTTGTATGTTTTTGTAGAGATGAGGTTTCA
CCATGTTGCTGAGGCTTGTCTCCAACTTCTGGGCTCAAGCTATCTGCCCG
CCTCGGCCCCGCAAAGTGCTAGGATTACAGGTGTGAGACACTGCGCCCAG
CCCATTTGTAACTTTATTGTTTTCTCTTACAGGCAAATGTTCTGAAAAAG
ACTCTGCATG

CCND1 (Cyclin D1)

Cyclin D1 belongs to the highly conserved cyclin family, whose members are characterized by a dramatic periodicity in protein abundance throughout the cell cycle. Cyclins function as regulators of CDK kinases. Different cyclins exhibit distinct expression and degradation patterns which contribute to the temporal coordination of each mitotic event. This cyclin forms a complex with and functions as a regulatory subunit of CDK4 or CDK6, which are required for cell cycle G1/S transition. Regulatory component of the cyclin D1-CDK4 complex is believed to phosphorylates/interact and inhibit tumor suppressor retinoblastoma protein, RB1 to regulate cell-cycle during G1/S transition as phosphorylation of RB1 allows dissociation of the transcription factor E2F from the RB/E2F complex and the subsequent transcription of E2F target genes which are responsible for the progression through the G1 phase. Further, CCND1 expression is believed to be regulated positively by Rb. Mutations, amplification and overexpression of CCND1 alters cell cycle progression and are observed frequently in a variety of tumors including mantle cell lymphoma (characterized by the t(11; 14) rearrangement) and other B-cell lymphomas.

Protein: Cyclin D1 Gene: CCND1 (Homo sapiens, chromosome 11, 69455873-69469242 [NCBI Reference Sequence: NC_{—}000009.12]; start site location: 69456082; strand: positive)

Gene Identification

| | |
|---|---|
| GeneID | 595 |
| HGNC | 1582 |
| HPRD | 01346 |
| MIM | 168461 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 12129 | | CGCTGCTACTGCGCCGACAGCCCTC | 133 |
| 12242 | | CGGCAGAATGGGCGCATTTCCAAGA | 612 |
| 12287 | | ACGCCACGAGGGCACCCACGGGCGGA | 637 |
| 12332 | | CGGTGACCGCGGCCTGGGCGGATGG | 2755 |
| 12388 | | CGGGACTCAGCGCGGCTGCGCGCCG | 2907 |

Targeted Shift Sequences

| | | Relative |
|---|---|---|
| Sequence | | upstream |
| ID | | location to gene |
| No: | Sequence (5′-3′) | start site |
| 12129 | CGCTGCTACTGCGCCGACAGCCCTC | 133 |
| 12130 | GCTGCTACTGCGCCGACAGC | 134 |
| 12131 | CTGCTACTGCGCCGACAGCC | 135 |
| 12132 | TGCTACTGCGCCGACAGCCC | 136 |
| 12133 | GCTACTGCGCCGACAGCCCT | 137 |
| 12134 | CTACTGCGCCGACAGCCCTC | 138 |
| 12135 | TACTGCGCCGACAGCCCTCT | 139 |
| 12136 | ACTGCGCCGACAGCCCTCTG | 140 |
| 12137 | CTGCGCCGACAGCCCTCTGG | 141 |
| 12138 | TGCGCCGACAGCCCTCTGGA | 142 |
| 12139 | GCGCCGACAGCCCTCTGGAG | 143 |
| 12140 | CGCCGACAGCCCTCTGGAGG | 144 |
| 12141 | GCCGACAGCCCTCTGGAGGC | 145 |
| 12142 | CCGACAGCCCTCTGGAGGCT | 146 |
| 12143 | CGACAGCCCTCTGGAGGCTC | 147 |
| 12144 | TCGCTGCTACTGCGCCGACA | 132 |
| 12145 | CTCGCTGCTACTGCGCCGAC | 131 |
| 12146 | GCTCGCTGCTACTGCGCCGA | 130 |
| 12147 | TGCTCGCTGCTACTGCGCCG | 129 |
| 12148 | CTGCTCGCTGCTACTGCGCC | 128 |
| 12149 | GCTGCTCGCTGCTACTGCGC | 127 |
| 12150 | TGCTGCTCGCTGCTACTGCG | 126 |
| 12151 | CTGCTGCTCGCTGCTACTGC | 125 |
| 12152 | TCTGCTGCTCGCTGCTACTG | 124 |
| 12153 | CTCTGCTGCTCGCTGCTACT | 123 |
| 12154 | ACTCTGCTGCTCGCTGCTAC | 122 |
| 12155 | GACTCTGCTGCTCGCTGCTA | 121 |
| 12156 | GGACTCTGCTGCTCGCTGCT | 120 |
| 12157 | CGGACTCTGCTGCTCGCTGC | 119 |
| 12158 | GCGGACTCTGCTGCTCGCTG | 118 |
| 12159 | TGCGGACTCTGCTGCTCGCT | 117 |
| 12160 | GTGCGGACTCTGCTGCTCGC | 116 |
| 12161 | CGTGCGGACTCTGCTGCTCG | 115 |
| 12162 | GCGTGCGGACTCTGCTGCTC | 114 |
| 12163 | AGCGTGCGGACTCTGCTGCT | 113 |
| 12164 | GAGCGTGCGGACTCTGCTGC | 112 |
| 12165 | GGAGCGTGCGGACTCTGCTG | 111 |
| 12166 | CGGAGCGTGCGGACTCTGCT | 110 |
| 12167 | CCGGAGCGTGCGGACTCTGC | 109 |
| 12168 | GCCGGAGCGTGCGGACTCTG | 108 |
| 12169 | CGCCGGAGCGTGCGGACTCT | 107 |
| 12170 | TCGCCGGAGCGTGCGGACTC | 106 |
| 12171 | CTCGCCGGAGCGTGCGGACT | 105 |
| 12172 | CCTCGCCGGAGCGTGCGGAC | 104 |
| 12173 | CCCTCGCCGGAGCGTGCGGA | 103 |
| 12174 | CCCCTCGCCGGAGCGTGCGG | 102 |
| 12175 | GCCCCTCGCCGGAGCGTGCG | 101 |
| 12176 | TGCCCCTCGCCGGAGCGTGC | 100 |
| 12177 | CTGCCCCTCGCCGGAGCGTG | 99 |
| 12178 | TCTGCCCCTCGCCGGAGCGT | 98 |
| 12179 | TTCTGCCCCTCGCCGGAGCG | 97 |
| 12180 | CTTCTGCCCCTCGCCGGAGC | 96 |
| 12181 | TCTTCTGCCCCTCGCCGGAG | 95 |
| 12182 | CTCTTCTGCCCCTCGCCGGA | 94 |
| 12183 | GCTCTTCTGCCCCTCGCCGG | 93 |
| 12184 | CGCTCTTCTGCCCCTCGCCG | 92 |
| 12185 | GCGCTCTTCTGCCCCTCGCC | 91 |
| 12186 | CGCGCTCTTCTGCCCCTCGC | 90 |
| 12187 | TCGCGCTCTTCTGCCCCTCG | 89 |
| 12188 | CTCGCGCTCTTCTGCCCCTC | 88 |
| 12189 | CCTCGCGCTCTTCTGCCCCT | 87 |
| 12190 | CCCTCGCGCTCTTCTGCCCC | 86 |
| 12191 | TCCCTCGCGCTCTTCTGCCC | 85 |
| 12192 | CTCCCTCGCGCTCTTCTGCC | 84 |
| 12193 | GCTCCCTCGCGCTCTTCTGC | 83 |
| 12194 | CGCTCCCTCGCGCTCTTCTG | 82 |
| 12195 | GCGCTCCCTCGCGCTCTTCT | 81 |
| 12196 | CGCGCTCCCTCGCGCTCTTC | 80 |
| 12197 | CCGCGCTCCCTCGCGCTCTT | 79 |
| 12198 | CCCGCGCTCCCTCGCGCTCT | 78 |
| 12199 | CCCCGCGCTCCCTCGCGCTC | 77 |
| 12200 | GCCCCGCGCTCCCTCGCGCT | 76 |
| 12201 | TGCCCCGCGCTCCCTCGCGC | 75 |
| 12202 | CTGCCCCGCGCTCCCTCGCG | 74 |
| 12203 | GCTGCCCCGCGCTCCCTCGC | 73 |
| 12204 | TGCTGCCCCGCGCTCCCTCG | 72 |
| 12205 | CTGCTGCCCCGCGCTCCCTC | 71 |
| 12206 | TCTGCTGCCCCGCGCTCCCT | 70 |
| 12207 | TTCTGCTGCCCCGCGCTCCC | 69 |
| 12208 | CTTCTGCTGCCCCGCGCTCC | 68 |
| 12209 | GCTTCTGCTGCCCCGCGCTC | 67 |
| 12210 | CGCTTCTGCTGCCCCGCGCT | 66 |
| 12211 | TCGCTTCTGCTGCCCCGCGC | 65 |
| 12212 | CTCGCTTCTGCTGCCCCGCG | 64 |
| 12213 | TCTCGCTTCTGCTGCCCCGC | 63 |
| 12214 | CTCTCGCTTCTGCTGCCCCG | 62 |
| 12215 | GCTCTCGCTTCTGCTGCCCC | 61 |
| 12216 | GGCTCTCGCTTCTGCTGCCC | 60 |
| 12217 | CGGCTCTCGCTTCTGCTGCC | 59 |
| 12218 | TCGGCTCTCGCTTCTGCTGC | 58 |
| 12219 | CTCGGCTCTCGCTTCTGCTG | 57 |
| 12220 | GCTCGGCTCTCGCTTCTGCT | 56 |
| 12221 | CGCTCGGCTCTCGCTTCTGC | 55 |
| 12222 | GCGCTCGGCTCTCGCTTCTG | 54 |
| 12223 | CGCGCTCGGCTCTCGCTTCT | 53 |
| 12224 | CCGCGCTCGGCTCTCGCTTC | 52 |
| 12225 | TCCGCGCTCGGCTCTCGCTT | 51 |
| 12226 | GTCCGCGCTCGGCTCTCGCT | 50 |
| 12227 | GGTCCGCGCTCGGCTCTCGC | 49 |
| 12228 | GGGTCCGCGCTCGGCTCTCG | 48 |
| 12229 | TGGGTCCGCGCTCGGCTCTC | 47 |
| 12230 | CTGGGTCCGCGCTCGGCTCT | 46 |
| 12231 | GCTGGGTCCGCGCTCGGCTC | 45 |
| 12232 | GGCTGGGTCCGCGCTCGGCT | 44 |
| 12233 | TGGCTGGGTCCGCGCTCGGC | 43 |
| 12234 | CTGGCTGGGTCCGCGCTCGG | 42 |
| 12235 | CCTGGCTGGGTCCGCGCTCG | 41 |
| 12236 | TCCTGGCTGGGTCCGCGCTC | 40 |
| 12237 | GTCCTGGCTGGGTCCGCGCT | 39 |
| 12238 | GGTCCTGGCTGGGTCCGCGC | 38 |
| 12239 | GGGTCCTGGCTGGGTCCGCG | 37 |
| 12240 | TGGGTCCTGGCTGGGTCCGC | 36 |
| 12241 | GTGGGTCCTGGCTGGGTCCG | 35 |
| 12242 | CGGCAGAATGGGCGCATTTCCAAGA | 612 |
| 12243 | GGCAGAATGGGCGCATTTCC | 613 |
| 12244 | GCAGAATGGGCGCATTTCCA | 614 |
| 12245 | CAGAATGGGCGCATTTCCAA | 615 |
| 12246 | AGAATGGGCGCATTTCCAAG | 616 |
| 12247 | GAATGGGCGCATTTCCAAGA | 617 |
| 12248 | AATGGGCGCATTTCCAAGAA | 618 |
| 12249 | ATGGGCGCATTTCCAAGAAC | 619 |
| 12250 | TGGGCGCATTTCCAAGAACG | 620 |
| 12251 | GGGCGCATTTCCAAGAACGC | 621 |
| 12252 | GGCGCATTTCCAAGAACGCC | 622 |
| 12253 | GCGCATTTCCAAGAACGCCA | 623 |
| 12254 | CGCATTTCCAAGAACGCCAC | 624 |
| 12255 | GCATTTCCAAGAACGCCACG | 625 |
| 12256 | CATTTCCAAGAACGCCACGA | 626 |
| 12257 | ATTTCCAAGAACGCCACGAG | 627 |
| 12258 | TTTCCAAGAACGCCACGAGG | 628 |
| 12259 | TTCCAAGAACGCCACGAGGG | 629 |
| 12260 | TCCAAGAACGCCACGAGGGC | 630 |
| 12261 | CCAAGAACGCCACGAGGGCA | 631 |
| 12262 | CAAGAACGCCACGAGGGCAC | 632 |
| 12263 | AAGAACGCCACGAGGGCACC | 633 |
| 12264 | AGAACGCCACGAGGGCACCC | 634 |
| 12265 | GAACGCCACGAGGGCACCCA | 635 |
| 12266 | AACGCCACGAGGGCACCCAC | 636 |
| 12267 | ACGCCACGAGGGCACCCACG | 637 |
| 12268 | CGCCACGAGGGCACCCACGG | 638 |
| 12269 | GCCACGAGGGCACCCACGGG | 639 |
| 12270 | CCACGAGGGCACCCACGGGC | 640 |
| 12271 | CACGAGGGCACCCACGGGCG | 641 |
| 12272 | ACGAGGGCACCCACGGGCGG | 642 |
| 12273 | CGAGGGCACCCACGGGCGGA | 643 |
| 12274 | GAGGGCACCCACGGGCGGAC | 644 |
| 12275 | AGGGCACCCACGGGCGGACA | 645 |
| 12276 | GGGCACCCACGGGCGGACAG | 646 |
| 12277 | GGCACCCACGGGCGGACAGA | 647 |
| 12278 | GCACCCACGGGCGGACAGAC | 648 |
| 12279 | CACCCACGGGCGGACAGACG | 649 |
| 12280 | ACCCACGGGCGGACAGACGG | 650 |
| 12281 | CCCACGGGCGGACAGACGGC | 651 |
| 12282 | CCACGGGCGGACAGACGGCC | 652 |
| 12283 | CACGGGCGGACAGACGGCCA | 653 |
| 12284 | CCGGCAGAATGGGCGCATTT | 611 |
| 12285 | GCCGGCAGAATGGGCGCATT | 610 |
| 12286 | AGCCGGCAGAATGGGCGCAT | 609 |
| 12287 | ACGCCACGAGGGCACCCACGGGCGGA | 637 |
| 12288 | CGCCACGAGGGCACCCACGG | 638 |
| 12289 | GCCACGAGGGCACCCACGGG | 639 |
| 12290 | CCACGAGGGCACCCACGGGC | 640 |
| 12291 | CACGAGGGCACCCACGGGCG | 641 |
| 12292 | ACGAGGGCACCCACGGGCGG | 642 |
| 12293 | CGAGGGCACCCACGGGCGGA | 643 |
| 12294 | GAGGGCACCCACGGGCGGAC | 644 |
| 12295 | AGGGCACCCACGGGCGGACA | 645 |
| 12296 | GGGCACCCACGGGCGGACAG | 646 |
| 12297 | GGCACCCACGGGCGGACAGA | 647 |
| 12298 | GCACCCACGGGCGGACAGAC | 648 |
| 12299 | CACCCACGGGCGGACAGACG | 649 |
| 12300 | ACCCACGGGCGGACAGACGG | 650 |
| 12301 | CCCACGGGCGGACAGACGGC | 651 |
| 12302 | CCACGGGCGGACAGACGGCC | 652 |
| 12303 | CACGGGCGGACAGACGGCCA | 653 |
| 12304 | AACGCCACGAGGGCACCCAC | 636 |
| 12305 | GAACGCCACGAGGGCACCCA | 635 |
| 12306 | AGAACGCCACGAGGGCACCC | 634 |
| 12307 | AAGAACGCCACGAGGGCACC | 633 |
| 12308 | CAAGAACGCCACGAGGGCAC | 632 |
| 12309 | CCAAGAACGCCACGAGGGCA | 631 |
| 12310 | TCCAAGAACGCCACGAGGGC | 630 |
| 12311 | TTCCAAGAACGCCACGAGGG | 629 |
| 12312 | TTTCCAAGAACGCCACGAGG | 628 |
| 12313 | ATTTCCAAGAACGCCACGAG | 627 |
| 12314 | CATTTCCAAGAACGCCACGA | 626 |
| 12315 | GCATTTCCAAGAACGCCACG | 625 |
| 12316 | CGCATTTCCAAGAACGCCAC | 624 |
| 12317 | GCGCATTTCCAAGAACGCCA | 623 |
| 12318 | GGCGCATTTCCAAGAACGCC | 622 |
| 12319 | GGGCGCATTTCCAAGAACGC | 621 |
| 12320 | TGGGCGCATTTCCAAGAACG | 620 |
| 12321 | ATGGGCGCATTTCCAAGAAC | 619 |
| 12322 | AATGGGCGCATTTCCAAGAA | 618 |
| 12323 | GAATGGGCGCATTTCCAAGA | 617 |
| 12324 | AGAATGGGCGCATTTCCAAG | 616 |
| 12325 | CAGAATGGGCGCATTTCCAA | 615 |
| 12326 | GCAGAATGGGCGCATTTCCA | 614 |
| 12327 | GGCAGAATGGGCGCATTTCC | 613 |
| 12328 | CGGCAGAATGGGCGCATTTC | 612 |
| 12329 | CCGGCAGAATGGGCGCATTT | 611 |
| 12330 | GCCGGCAGAATGGGCGCATT | 610 |
| 12331 | AGCCGGCAGAATGGGCGCAT | 609 |
| 12332 | CGGTGACCGCGGCCTGGGCGGATGG | 2755 |
| 12333 | GGTGACCGCGGCCTGGGCGG | 2756 |
| 12334 | GTGACCGCGGCCTGGGCGGA | 2757 |
| 12335 | TGACCGCGGCCTGGGCGGAT | 2758 |
| 12336 | GACCGCGGCCTGGGCGGATG | 2759 |
| 12337 | ACCGCGGCCTGGGCGGATGG | 2760 |
| 12338 | CCGCGGCCTGGGCGGATGGT | 2761 |
| 12339 | CGCGGCCTGGGCGGATGGTC | 2762 |
| 12340 | GCGGCCTGGGCGGATGGTCG | 2763 |
| 12341 | CGGCCTGGGCGGATGGTCGG | 2764 |
| 12342 | GGCCTGGGCGGATGGTCGGT | 2765 |
| 12343 | GCCTGGGCGGATGGTCGGTC | 2766 |
| 12344 | CCTGGGCGGATGGTCGGTCA | 2767 |
| 12345 | CTGGGCGGATGGTCGGTCAG | 2768 |
| 12346 | TGGGCGGATGGTCGGTCAGG | 2769 |
| 12347 | CCGGTGACCGCGGCCTGGGC | 2754 |
| 12348 | CCCGGTGACCGCGGCCTGGG | 2753 |
| 12349 | CCCCGGTGACCGCGGCCTGG | 2752 |
| 12350 | GCCCCGGTGACCGCGGCCTG | 2751 |
| 12351 | CGCCCCGGTGACCGCGGCCT | 2750 |
| 12352 | CCGCCCCGGTGACCGCGGCC | 2749 |
| 12353 | CCCGCCCCGGTGACCGCGGC | 2748 |
| 12354 | CCCCGCCCCGGTGACCGCGG | 2747 |
| 12355 | CCCCCGCCCCGGTGACCGCG | 2746 |
| 12356 | GCCCCCGCCCCGGTGACCGC | 2745 |
| 12357 | GGCCCCCGCCCCGGTGACCG | 2744 |
| 12358 | TGGCCCCCGCCCCGGTGACC | 2743 |
| 12359 | CTGGCCCCCGCCCCGGTGAC | 2742 |
| 12360 | CCTGGCCCCCGCCCCGGTGA | 2741 |
| 12361 | CCCTGGCCCCCGCCCCGGTG | 2740 |
| 12362 | CCCCTGGCCCCCGCCCCGGT | 2739 |
| 12363 | CCCCCTGGCCCCCGCCCCGG | 2738 |
| 12364 | GCCCCCTGGCCCCCGCCCCG | 2737 |
| 12365 | CGCCCCCTGGCCCCCGCCCC | 2736 |
| 12366 | TCGCCCCCTGGCCCCCGCCC | 2735 |
| 12367 | CTCGCCCCCTGGCCCCCGCC | 2734 |
| 12368 | CCTCGCCCCCTGGCCCCCGC | 2733 |
| 12369 | TCCTCGCCCCCTGGCCCCCG | 2732 |
| 12370 | TTCCTCGCCCCCTGGCCCCC | 2731 |
| 12371 | TTTCCTCGCCCCCTGGCCCC | 2730 |
| 12372 | CTTTCCTCGCCCCCTGGCCC | 2729 |
| 12373 | GCTTTCCTCGCCCCCTGGCC | 2728 |
| 12374 | CGCTTTCCTCGCCCCCTGGC | 2727 |
| 12375 | ACGCTTTCCTCGCCCCCTGG | 2726 |
| 12376 | CACGCTTTCCTCGCCCCCTG | 2725 |
| 12377 | TCACGCTTTCCTCGCCCCCT | 2724 |
| 12378 | TTCACGCTTTCCTCGCCCCC | 2723 |
| 12379 | CTTCACGCTTTCCTCGCCCC | 2722 |
| 12380 | CCTTCACGCTTTCCTCGCCC | 2721 |
| 12381 | ACCTTCACGCTTTCCTCGCC | 2720 |
| 12382 | CACCTTCACGCTTTCCTCGC | 2719 |
| 12383 | TCACCTTCACGCTTTCCTCG | 2718 |
| 12384 | ATCACCTTCACGCTTTCCTC | 2717 |
| 12385 | AATCACCTTCACGCTTTCCT | 2716 |
| 12386 | AAATCACCTTCACGCTTTCC | 2715 |
| 12387 | GAAATCACCTTCACGCTTTC | 2714 |
| 12388 | CGGGACTCAGCGCGGCTGCGCGCCG | 2907 |
| 12389 | GGGACTCAGCGCGGCTGCGC | 2908 |
| 12390 | GGACTCAGCGCGGCTGCGCG | 2909 |
| 12391 | GACTCAGCGCGGCTGCGCGC | 2910 |
| 12392 | ACTCAGCGCGGCTGCGCGCC | 2911 |
| 12393 | CTCAGCGCGGCTGCGCGCCG | 2912 |
| 12394 | TCAGCGCGGCTGCGCGCCGC | 2913 |
| 12395 | CAGCGCGGCTGCGCGCCGCG | 2914 |
| 12396 | AGCGCGGCTGCGCGCCGCGG | 2915 |
| 12397 | GCGCGGCTGCGCGCCGCGGG | 2916 |
| 12398 | CGCGGCTGCGCGCCGCGGGG | 2917 |
| 12399 | GCGGCTGCGCGCCGCGGGGC | 2918 |
| 12400 | CGGCTGCGCGCCGCGGGGCT | 2919 |
| 12401 | GGCTGCGCGCCGCGGGGCTC | 2920 |
| 12402 | GCTGCGCGCCGCGGGGCTCG | 2921 |
| 12403 | CTGCGCGCCGCGGGGCTCGG | 2922 |
| 12404 | TGCGCGCCGCGGGGCTCGGG | 2923 |
| 12405 | GCGCGCCGCGGGGCTCGGGG | 2924 |
| 12406 | CGCGCCGCGGGGCTCGGGGC | 2925 |
| 12407 | GCGCCGCGGGGCTCGGGGCT | 2926 |
| 12408 | CGCCGCGGGGCTCGGGGCTT | 2927 |
| 12409 | GCCGCGGGGCTCGGGGCTTG | 2928 |
| 12410 | CCGCGGGGCTCGGGGCTTGG | 2929 |
| 12411 | CGCGGGGCTCGGGGCTTGGG | 2930 |
| 12412 | GCGGGGCTCGGGGCTTGGGT | 2931 |
| 12413 | CGGGGCTCGGGGCTTGGGTT | 2932 |
| 12414 | GGGGCTCGGGGCTTGGGTTG | 2933 |
| 12415 | GGGCTCGGGGCTTGGGTTGG | 2934 |
| 12416 | GGCTCGGGGCTTGGGTTGGG | 2935 |
| 12417 | GCTCGGGGCTTGGGTTGGGG | 2936 |
| 12418 | CTCGGGGCTTGGGTTGGGGG | 2937 |
| 12419 | TCGGGGCTTGGGTTGGGGGC | 2938 |
| 12420 | CGGGGCTTGGGTTGGGGGCG | 2939 |
| 12421 | CCGGGACTCAGCGCGGCTGC | 2906 |
| 12422 | CCCGGGACTCAGCGCGGCTG | 2905 |
| 12423 | CCCCGGGACTCAGCGCGGCT | 2904 |
| 12424 | ACCCCGGGACTCAGCGCGGC | 2903 |
| 12425 | GACCCCGGGACTCAGCGCGG | 2902 |
| 12426 | AGACCCCGGGACTCAGCGCG | 2901 |
| 12427 | CAGACCCCGGGACTCAGCGC | 2900 |
| 12428 | GCAGACCCCGGGACTCAGCG | 2899 |
| 12429 | CGCAGACCCCGGGACTCAGC | 2898 |
| 12430 | ACGCAGACCCCGGGACTCAG | 2897 |
| 12431 | GACGCAGACCCCGGGACTCA | 2896 |
| 12432 | CGACGCAGACCCCGGGACTC | 2895 |
| 12433 | GCGACGCAGACCCCGGGACT | 2894 |
| 12434 | CGCGACGCAGACCCCGGGAC | 2893 |
| 12435 | CCGCGACGCAGACCCCGGGA | 2892 |
| 12436 | GCCGCGACGCAGACCCCGGG | 2891 |
| 12437 | CGCCGCGACGCAGACCCCGG | 2890 |
| 12438 | GCGCCGCGACGCAGACCCCG | 2889 |
| 12439 | CGCGCCGCGACGCAGACCCC | 2888 |
| 12440 | GCGCGCCGCGACGCAGACCC | 2887 |
| 12441 | GGCGCGCCGCGACGCAGACC | 2886 |
| 12442 | CGGCGCGCCGCGACGCAGAC | 2885 |
| 12443 | CCGGCGCGCCGCGACGCAGA | 2884 |
| 12444 | ACCGGCGCGCCGCGACGCAG | 2883 |
| 12445 | AACCGGCGCGCCGCGACGCA | 2882 |
| 12446 | GAACCGGCGCGCCGCGACGC | 2881 |
| 12447 | GGAACCGGCGCGCCGCGACG | 2880 |
| 12448 | AGGAACCGGCGCGCCGCGAC | 2879 |
| 12449 | CAGGAACCGGCGCGCCGCGA | 2878 |
| 12450 | TCAGGAACCGGCGCGCCGCG | 2877 |
| 12451 | TTCAGGAACCGGCGCGCCGC | 2876 |
| 12452 | ATTCAGGAACCGGCGCGCCG | 2875 |
| 12453 | CATTCAGGAACCGGCGCGCC | 2874 |
| 12454 | TCATTCAGGAACCGGCGCGC | 2873 |
| 12455 | TTCATTCAGGAACCGGCGCG | 2872 |
| 12456 | GTTCATTCAGGAACCGGCGC | 2871 |
| 12457 | CGTTCATTCAGGAACCGGCG | 2870 |
| 12458 | GCGTTCATTCAGGAACCGGC | 2869 |
| 12459 | CGCGTTCATTCAGGAACCGG | 2868 |
| 12460 | GCGCGTTCATTCAGGAACCG | 2867 |
| 12461 | AGCGCGTTCATTCAGGAACC | 2866 |
| 12462 | GAGCGCGTTCATTCAGGAAC | 2865 |
| 12463 | GGAGCGCGTTCATTCAGGAA | 2864 |
| 12464 | GGGAGCGCGTTCATTCAGGA | 2863 |
| 12465 | AGGGAGCGCGTTCATTCAGG | 2862 |
| 12466 | AAGGGAGCGCGTTCATTCAG | 2861 |
| 12467 | GAAGGGAGCGCGTTCATTCA | 2860 |
| 12468 | GGAAGGGAGCGCGTTCATTC | 2859 |
| 12469 | GGGAAGGGAGCGCGTTCATT | 2858 |

Hot Zones (Relative upstream location to gene start site)
1-250
550-700
2700-2300

### Examples

Genetic Code (5′ Upstream Region)

(SEQ ID NO: 13676)

GGCCCTGCCGCCCAGAACTCGCTGGGCAAGTCGTGCCCCGCGTGAACACA
CAGAAGGGGCTTGGGGACCGAGCGCGGCCCATCAGTCCCTCAGACCCTGA
GGACCCAGAATTCCCTAAGGGGTCCGAATCCGAGTCCTGCCCCCAGCCCT
TAAGGCACGGGCTCCAGGGACCCCAGGGGAAGGGCGCGGGGCATTAGGTA
CGCAACCCGTTTCCCCGCACCTGGAAAAAAACTCCCTTTCCCTCCCCTCC
CCTGCTTGTTGAGTGTCCGGATAACCAGAACTCTAAGGCGCCCCGTAATA
ACGACCCCGCTGTCCCTCCACCCACCCCCAAGTGCCAAAGCGAGGGATGG
AAGCGCTTTCAAGCGTTCCAAGGGCATTGAGGAGCGAGCTGGAGAGGCGC
GGGGATGCGGGGTCCTCCCCGCAGTCTTCCGGAAAGGGCGGGGGAGGGCG
CGGCAAGTTCCGGAGTGGGGCATGCCGTGGGAGCCCACGAGGGCCTCAGC
GCGGATCCTCCGCCGGAAAACCGGCTCCCGCGAGCCGCCGCCGCAGGTTT
CCTAGGCCCCGCGAGTCCCGCAGCGAAGCCCTGCGTCTCCGTCCGACGCG
GGGGTCTGCTCAGCCTCGGGTGGGCCGCGGCCAGGCCTGACTGCGGGGGA
GAGGGCCGAACGTGACCTCCGAGGTCACCCCCAGCCAGCTTTCTCTCCTG
TGGTCGGAAGTGGTTTTCTTCTCGATCTGGGCGCCTACTCCCCACCACTT
GGTCTGAGAGGGGCTGGGGCCGGAAGGCCAGGGAATCTCTGGTGGATTTG
GGGGTTCATATTGCTCAGGGTACCAGCCGATGCGTTTTGAGGGGCGGGAG
TCGAGGAATTAGAATCGCCTTTAACCCTCAAGAGTTGCGCCTTCAGCCTC
GGGATCCCAGATGCGTCGTTGGAGCCAGGGCCGCCCCCCTACCTGTTGGG
TTTGCGTTTTAACTCCAGCGCACACCTTGCCGGCAGCCCTCGGAGCTAGG
GGAGGGGTCTCGTTTCCCCGCAGCCCGCCGGACAGACGACTGGGGCACGG
GAGGGGCGGTGGCAGGGTGGTCTGTGTGTGGCTGAAACTAATTGATCTGG
AGCGGAAACGCACGTCTGCGGTTGGGGCGATGGGGGGGGCGGTGCGGCTG
TCCATGTGCCGAGCGTGTGGCTGTCTCGGGTGGGCACTGGGGCCGGAGTT
CGCCCCGGCCCACCTCGCAGTTTTGGGGCGCCTGGGATCGGCGCTACGTA
AGCGAAGCAGAGCTGCCATAGCACGTGGGCCGCCACGCGCACCCCAAAAG
CAAGCAGTGTGGGGGGAAGGGGAGCTCGAGCGCCTTCGGAGCCCAGGGGC
CGGCTTTCGGAAGCGTTTTCCCGGGCGACTTAAGGGCTTAACAATGGAAA
ACTCGCGGAGCCTGAGCCAAGTCCTTTCAAGTCGCCGCCAGGTATGCGGC
TGCAGGTGACCCCACCTGGGTGCGCCCGCCCGCCAGCCGCCCTGGTGGAA
AAGCGGGTGCGGGAGGTCGCTGGCGAAAGGTCGGGACTGGTCCCTGCACC
ACCCGCCCCCAACCCAAGCCCCGAGCCCCGCGGCGCGCAGCCGCGCTGAG
TCCCGGGGTCTGCGTCGCGGCGCGCCGGTTCCTGAATGAACGCGCTCCCT
TCCCCCGCCTGAATGAAGGTTCCCACAGCCAGGGACGGTGGCGAACACGC
GCCTGCAGCGGAATTCGCTTTCTCCTGACCGACCATCCGCCCAGGCCGCG
GTCACCGGGGCGGGGGCCAGGGGGCGAGGAAAGCGTGAAGGTGATTTCAG
TTAATTTTGGATTTTCTTTCAAACAACGTGGTTACCCTCCCGACTGGGCC
ACTTGCCCTTTGTCTCCAAATGGTCACCAAGAAATAAGAACAGAGCACTT
TAAATGAGCCCAGAATCCGCAGTTCCTGCTTCGTGGTGGGTTTTAAGAAG
ACAGTGTAAAGTAAAACTGCAACCGAAAAGTTTTTTAAAGTTGCTTTTCT
CTTTGGAAAAAATAAAATCAAAATGCTTTCTCTGCGCTTCTTGAAGCAAT
GACCCTCAAAAGCCCAGAGGTATTGGCCCCCTCGGGGGACCCGGGGGCCG
CCAAGCAGGGTTCCCCCAGGTGGGGGCTGGGCAGCTGGCGCTCCCCGCCG
GGCCCCAAATTCCAGCGCCGGGCCCCAAATTCCAGCGCCTCCCCCGCGGG
TTCCTGGACGGCTCTTTACGCTCGCTAACCGGGCTTGCAATTTTGCGCTC
GTCCCTGAGCCGGGAAATCAACGAAGTTCCTAGTCGAGATCTGCCCGGTC
CGCCTAGTAACAGCGCCGCGCCCCCATTGGCTCATGCTAATTCCAGTTTC
CTCTGTCTTGCGCCCGGGATGGGGGGGTGAAGCTCCCTCCTGGACCCAGA
GCCGGTTGTGCCGGAGTGGGCGAGCCTCTTTATGCCCTGCTGCCCCTAGC
CGACTTCGGCCCGCTTCGCGCCTCGGGCTGGGCCAGGGCGCACGCGGGGC
TCGGGGCCCCTCGCCCCACGGGATGGGAGAGGCCGGGTGATAGCTCCGGG
CCCCATAAATCATCCAGGCGGCCGCCGGGTCGGGATTTTATGAATGAAAA
AGCAGCTGGGCCGCCCTTGTGCGCGGGCTGATGCTCTGAGGCTTGGCTAT
GCGGGGGCCAACGCGATTGTGGGTGCTCGGGGAGTGGGGGGGGGCACGAC
CGTAGGTGCTCCCTGCTGGGGCAACCCATCGCTCCCCATGCGGAATCCGG
GGGTAATTACCCCCCCAGGACCCGGAATATTAGTAATCCTAATTCCCGGC
GGGGGAGGGGGCGCGGGAGGAATTCACCCTGAAAGGTGGGGGTGGGGGGG
GTCGCATCTTGCTGTGAGCACCCTGGCGAAGGGGAGAGGGCTTTTTCTAT
CAGTTTTCTTTGAGCTTTTACTGTTAAGAGGGTACGGTGGTTTGATGACA
CTGAACTATATTCAAAAGGAAGTAAATGAACAGTTTTCTTAATTTGGGGC
AGGTACTGTAAAAATAAAAACAAAAGTTAAGACAGTAAAATGTCCTTTTA
TTTTTTAATGCACCAAAGAGACAGAACCTGTAATTTTAAAAACTGTGTAT
TTTAATTTACATCTGCTTAAGTTTGCGATAATATTGGGGACCCTCTCATG
TAACCACGAACACCTATCGATTTTGCTAAAAATCAGATCAGTACACTCGT
TTGTTTAATTGATAATTGTTCTGAATTATGCCGGCTCCTGCCAGCCCCCT
CACGCTCACGAATTCAGTCCCAGGGCAAATTCTAAAGGTGAAGGGACGTC
TACACCCCCAACAAAACCAATTAGGAACCTTCGGTGGTCTTGTCCCAGGC
AGAGGGGACTAATATTTCCAGCAATTTAATTTCTTTTTTAATTAAAAAAA
ATGAGTCAGAATGGAGATCACTGTTTCTCAGCTTTCCATTCAGAGGTGTG
TTTCTCCCGGTTAAATTGCCGGCACGGGAAGGGAGGGGGTGCAGTTGGGG
ACCCCCGCAAGGACCGACTGGTCAAGGTAGGAAGGCAGCCCGAAGAGTCT
CCAGGCTAGAAGGACAAGATGAAGGAAATGCTGGCCACCATCTTGGGCTG
CTGCTGGAATTTTCGGGCATTTATTTTATTTTATTTTTTGAGCGAGCGCA
TGCTAAGCTGAAATCCCTTTAACTTTTAGGGTTACCCCCTTGGGCATTTG
CAACGACGCCCCTGTGCGCCGGAATGAAACTTGCACAGGGGTTGTGTGCC
CGGTCCTCCCCGTCCTTGCATGCTAAATTAGTTCTTGCAATTTACACGTG
TTAATGAAAATGAAAGAAGATGCAGTCGCTGAGATTCTTTGGCCGTCTGT
CCGCCCGTGGGTGCCCTCGTGGCGTTCTTGGAAATGCGCCCATTCTGCCG
GCTTGGATATGGGGTGTCGCCGCGCCCCAGTCACCCCTTCTCGTGGTCTC
CCCAGGCTGCGTGTGGCCTGCCGGCCTTCCTAGTTGTCCCCTACTGCAGA
GCCACCTCCACCTCACCCCCTAAATCCCGGGGGACCCACTCGAGGCGGAC
GGGGCCCCCTGCACCCCTCTTCCCTGGCGGGGAGAAAGGCTGCAGCGGGG
CGATTTGCATTTCTATGAAAACCGGACTACAGGGGCAACTCCGCCGCAGG
GCAGGCGCGGCGCCTCAGGGATGGCTTTTGGGCTCTGCCCCTCGCTGCTC
CCGGCGTTTGGCGCCCGCGCCCCCTCCCCCTGCGCCCGCCCCCGCCCCCC
TCCCGCTCCCATTCTCTGCCGGGCTTTGATCTTTGCTTAACAACAGTAAC
GTCACACGGACTACAGGGGAGTTTTGTTGAAGTTGCAAAGTCCTGGAGCC
TCCAGAGGGCTGTCGGCGCAGTAGCAGCGAGCAGCAGAGTCCGCACGCTC
CGGCGAGGGGCAGAAGAGCGCGAGGGAGCGCGGGGCAGCAGAAGCGAGAG
CCGAGCGCGGACCCAGCCAGGACCCACAGCCCTCCCCAGCTGCCCAGGAA
GAGCCCCAGCCATG

MIF1

MIF1 (macrophage migration inhibitory factor 1) is a lymphokine involved in cell-mediated immunity, immunoregulation, and inflammation. MIF forms a homotrimer with three catalytic sites. The MIF homotrimer can enter a cell via endocytosis where it interacts with intracellular proteins. This interaction results in downregulating MAPK signals leading to activation of Cyclin D1 and subsequent cellular proliferation. Depending on the cellular environment, MIF may also have antioxidant activity which would inhibit apoptosis. Apoptosis can also be inhibited via a MIF-CD74 complex. MIF has been associated with inflammation, including rheumatoid arthritis, sepsis, and cancer.

Protein: MIF1 Gene: MIF1 (Homo sapiens, chromosome 22, 24236565-24237409[NCBI Reference Sequence: NC_{—}000022.10]; start site location: 24236662; strand: positive)

Gene Identification

| | |
|---|---|
| GeneID | 4282 |
| HGNC | 7097 |
| HPRD | 01091 |
| MIM | 153620 |

Targeted Sequences

| | | | Relative upstream |
|---|---|---|---|
| Sequence | Design | | location to gene start |
| ID No: | ID | Sequence (5′-3′) | site |
| 12470 | | GACCCGCGCAGAGGCACAGACGC | 22 |
| 12490 | | CGCCACCGCCGGCGCCAGGCCCCGCCCCCGCG | 123 |
| 12701 | | CGTTCCTCCAGCAACCGCCGCTAAGCCCGGCG | 238 |
| 12902 | | CGCCTGCCTCGGCTCGACCCCCGCAG | 182 |
| 13123 | | CGGCTAGAAATCGGCCTGTTCCGGCCTCGCCT | 297 |
| 13174 | | CGGGGGTGGGGATGCGGCGGTGAACCCG | 384 |
| 13175 | | CGCGGCAGGTGAGAGGGGAGCTGCCCCTGCG | 568 |
| 13176 | | CGCGTGCACGTGTGTCCACATGAGTGC | 3656 |
| 13203 | MIF1_1 | CGCCACCGCCGGCGCCAGGCCCCGCC | 117 |
| 13414 | MIF1_2 | CGCGGCAGGTGAGAGGGGAGCTGCCC | 563 |

Target Shift Sequences

| | | Relative |
|---|---|---|
| | | upstream |
| | | location |
| | | to gene |
| Sequence | | start |
| ID No: | Sequence (5′-3′) | site |
| 12470 | GACCCGCGCAGAGGCACAGACGC | 20 |
| 12471 | ACCCGCGCAGAGGCACAGAC | 21 |
| 12472 | CCCGCGCAGAGGCACAGACG | 22 |
| 12473 | CCGCGCAGAGGCACAGACGC | 23 |
| 12474 | CGCGCAGAGGCACAGACGCA | 24 |
| 12475 | GCGCAGAGGCACAGACGCAC | 25 |
| 12476 | CGCAGAGGCACAGACGCACG | 26 |
| 12477 | GCAGAGGCACAGACGCACGC | 27 |
| 12478 | CAGAGGCACAGACGCACGCG | 28 |
| 12479 | AGAGGCACAGACGCACGCGC | 29 |
| 12480 | GAGGCACAGACGCACGCGCC | 30 |
| 12481 | AGGCACAGACGCACGCGCCG | 31 |
| 12482 | GGCACAGACGCACGCGCCGC | 32 |
| 12483 | GCACAGACGCACGCGCCGCG | 33 |
| 12484 | CACAGACGCACGCGCCGCGG | 34 |
| 12485 | ACAGACGCACGCGCCGCGGC | 35 |
| 12486 | CAGACGCACGCGCCGCGGCC | 36 |
| 12487 | AGACCCGCGCAGAGGCACAG | 19 |
| 12488 | GAGACCCGCGCAGAGGCACA | 18 |
| 12489 | GGAGACCCGCGCAGAGGCAC | 17 |
| 12490 | CGCCACCGCCGGCGCCAGGC | 112 |
| | CCCGCCCCCGCG | |
| 12491 | GCCACCGCCGGCGCCAGGCC | 113 |
| 12492 | CCACCGCCGGCGCCAGGCCC | 114 |
| 12493 | CACCGCCGGCGCCAGGCCCC | 115 |
| 12494 | ACCGCCGGCGCCAGGCCCCG | 116 |
| 12495 | CCGCCGGCGCCAGGCCCCGC | 117 |
| 12496 | CGCCGGCGCCAGGCCCCGCC | 118 |
| 12497 | GCCGGCGCCAGGCCCCGCCC | 119 |
| 12498 | CCGGCGCCAGGCCCCGCCCC | 120 |
| 12499 | CGGCGCCAGGCCCCGCCCCC | 121 |
| 12500 | GGCGCCAGGCCCCGCCCCCG | 122 |
| 12501 | GCGCCAGGCCCCGCCCCCGC | 123 |
| 12502 | CGCCAGGCCCCGCCCCCGCG | 124 |
| 12503 | GCCAGGCCCCGCCCCCGCGA | 125 |
| 12504 | CCAGGCCCCGCCCCCGCGAG | 126 |
| 12505 | CAGGCCCCGCCCCCGCGAGG | 127 |
| 12506 | AGGCCCCGCCCCCGCGAGGC | 128 |
| 12507 | GGCCCCGCCCCCGCGAGGCT | 129 |
| 12508 | GCCCCGCCCCCGCGAGGCTG | 130 |
| 12509 | CCCCGCCCCCGCGAGGCTGC | 131 |
| 12510 | CCCGCCCCCGCGAGGCTGCG | 132 |
| 12511 | CCGCCCCCGCGAGGCTGCGG | 133 |
| 12512 | CGCCCCCGCGAGGCTGCGGC | 134 |
| 12513 | GCCCCCGCGAGGCTGCGGCT | 135 |
| 12514 | CCCCCGCGAGGCTGCGGCTC | 136 |
| 12515 | CCCCGCGAGGCTGCGGCTCC | 137 |
| 12516 | CCCGCGAGGCTGCGGCTCCG | 138 |
| 12517 | CCGCGAGGCTGCGGCTCCGC | 139 |
| 12518 | CGCGAGGCTGCGGCTCCGCC | 140 |
| 12519 | GCGAGGCTGCGGCTCCGCCC | 141 |
| 12520 | CGAGGCTGCGGCTCCGCCCC | 142 |
| 12521 | GAGGCTGCGGCTCCGCCCCG | 143 |
| 12522 | AGGCTGCGGCTCCGCCCCGA | 144 |
| 12523 | GGCTGCGGCTCCGCCCCGAG | 145 |
| 12524 | GCTGCGGCTCCGCCCCGAGT | 146 |
| 12525 | CTGCGGCTCCGCCCCGAGTG | 147 |
| 12526 | TGCGGCTCCGCCCCGAGTGG | 148 |
| 12527 | GCGGCTCCGCCCCGAGTGGG | 149 |
| 12528 | CGGCTCCGCCCCGAGTGGGG | 150 |
| 12529 | GGCTCCGCCCCGAGTGGGGA | 151 |
| 12530 | GCTCCGCCCCGAGTGGGGAA | 152 |
| 12531 | CTCCGCCCCGAGTGGGGAAG | 153 |
| 12532 | TCCGCCCCGAGTGGGGAAGT | 154 |
| 12533 | CCGCCCCGAGTGGGGAAGTC | 155 |
| 12534 | CGCCCCGAGTGGGGAAGTCA | 156 |
| 12535 | GCCCCGAGTGGGGAAGTCAC | 157 |
| 12536 | CCCCGAGTGGGGAAGTCACC | 158 |
| 12537 | CCCGAGTGGGGAAGTCACCG | 159 |
| 12538 | CCGAGTGGGGAAGTCACCGC | 160 |
| 12539 | CGAGTGGGGAAGTCACCGCC | 161 |
| 12540 | GAGTGGGGAAGTCACCGCCT | 162 |
| 12541 | AGTGGGGAAGTCACCGCCTG | 163 |
| 12542 | GTGGGGAAGTCACCGCCTGC | 164 |
| 12543 | TGGGGAAGTCACCGCCTGCC | 165 |
| 12544 | GGGGAAGTCACCGCCTGCCT | 166 |
| 12545 | GGGAAGTCACCGCCTGCCTC | 167 |
| 12546 | GGAAGTCACCGCCTGCCTCG | 168 |
| 12547 | GAAGTCACCGCCTGCCTCGG | 169 |
| 12548 | AAGTCACCGCCTGCCTCGGC | 170 |
| 12549 | AGTCACCGCCTGCCTCGGCT | 171 |
| 12550 | GTCACCGCCTGCCTCGGCTC | 172 |
| 12551 | TCACCGCCTGCCTCGGCTCG | 173 |
| 12552 | CACCGCCTGCCTCGGCTCGA | 174 |
| 12553 | ACCGCCTGCCTCGGCTCGAC | 175 |
| 12554 | CCGCCTGCCTCGGCTCGACC | 176 |
| 12555 | CGCCTGCCTCGGCTCGACCC | 177 |
| 12556 | GCCTGCCTCGGCTCGACCCC | 178 |
| 12557 | CCTGCCTCGGCTCGACCCCC | 179 |
| 12558 | CTGCCTCGGCTCGACCCCCG | 180 |
| 12559 | TGCCTCGGCTCGACCCCCGC | 181 |
| 12560 | GCCTCGGCTCGACCCCCGCA | 182 |
| 12561 | CCTCGGCTCGACCCCCGCAG | 183 |
| 12562 | CTCGGCTCGACCCCCGCAGG | 184 |
| 12563 | TCGGCTCGACCCCCGCAGGG | 185 |
| 12564 | CGGCTCGACCCCCGCAGGGC | 186 |
| 12565 | GGCTCGACCCCCGCAGGGCA | 187 |
| 12566 | GCTCGACCCCCGCAGGGCAG | 188 |
| 12567 | CTCGACCCCCGCAGGGCAGG | 189 |
| 12568 | TCGACCCCCGCAGGGCAGGA | 190 |
| 12569 | CGACCCCCGCAGGGCAGGAC | 191 |
| 12570 | GACCCCCGCAGGGCAGGACC | 192 |
| 12571 | ACCCCCGCAGGGCAGGACCC | 193 |
| 12572 | CCCCCGCAGGGCAGGACCCT | 194 |
| 12573 | CCCCGCAGGGCAGGACCCTG | 195 |
| 12574 | CCCGCAGGGCAGGACCCTGG | 196 |
| 12575 | CCGCAGGGCAGGACCCTGGG | 197 |
| 12576 | CGCAGGGCAGGACCCTGGGC | 198 |
| 12577 | GCAGGGCAGGACCCTGGGCG | 199 |
| 12578 | CAGGGCAGGACCCTGGGCGA | 200 |
| 12579 | AGGGCAGGACCCTGGGCGAC | 201 |
| 12580 | GGGCAGGACCCTGGGCGACT | 202 |
| 12581 | GGCAGGACCCTGGGCGACTC | 203 |
| 12582 | GCAGGACCCTGGGCGACTCC | 204 |
| 12583 | CAGGACCCTGGGCGACTCCG | 205 |
| 12584 | AGGACCCTGGGCGACTCCGC | 206 |
| 12585 | GGACCCTGGGCGACTCCGCC | 207 |
| 12586 | GACCCTGGGCGACTCCGCCC | 208 |
| 12587 | ACCCTGGGCGACTCCGCCCG | 209 |
| 12588 | CCCTGGGCGACTCCGCCCGT | 210 |
| 12589 | CCTGGGCGACTCCGCCCGTT | 211 |
| 12590 | CTGGGCGACTCCGCCCGTTC | 212 |
| 12591 | TGGGCGACTCCGCCCGTTCC | 213 |
| 12592 | GGGCGACTCCGCCCGTTCCT | 214 |
| 12593 | GGCGACTCCGCCCGTTCCTC | 215 |
| 12594 | GCGACTCCGCCCGTTCCTCC | 216 |
| 12595 | CGACTCCGCCCGTTCCTCCA | 217 |
| 12596 | GACTCCGCCCGTTCCTCCAG | 218 |
| 12597 | ACTCCGCCCGTTCCTCCAGC | 219 |
| 12598 | CTCCGCCCGTTCCTCCAGCA | 220 |
| 12599 | TCCGCCCGTTCCTCCAGCAA | 221 |
| 12600 | CCGCCCGTTCCTCCAGCAAC | 222 |
| 12601 | CGCCCGTTCCTCCAGCAACC | 223 |
| 12602 | GCCCGTTCCTCCAGCAACCG | 224 |
| 12603 | CCCGTTCCTCCAGCAACCGC | 225 |
| 12604 | CCGTTCCTCCAGCAACCGCC | 226 |
| 12605 | CGTTCCTCCAGCAACCGCCG | 227 |
| 12606 | GTTCCTCCAGCAACCGCCGC | 228 |
| 12607 | TTCCTCCAGCAACCGCCGCT | 229 |
| 12608 | TCCTCCAGCAACCGCCGCTA | 230 |
| 12609 | CCTCCAGCAACCGCCGCTAA | 231 |
| 12610 | CTCCAGCAACCGCCGCTAAG | 232 |
| 12611 | TCCAGCAACCGCCGCTAAGC | 233 |
| 12612 | CCAGCAACCGCCGCTAAGCC | 234 |
| 12613 | CAGCAACCGCCGCTAAGCCC | 235 |
| 12614 | AGCAACCGCCGCTAAGCCCG | 236 |
| 12615 | GCAACCGCCGCTAAGCCCGG | 237 |
| 12616 | CAACCGCCGCTAAGCCCGGC | 238 |
| 12617 | AACCGCCGCTAAGCCCGGCG | 239 |
| 12618 | ACCGCCGCTAAGCCCGGCGC | 240 |
| 12619 | CCGCCGCTAAGCCCGGCGCA | 241 |
| 12620 | CGCCGCTAAGCCCGGCGCAC | 242 |
| 12621 | GCCGCTAAGCCCGGCGCACC | 243 |
| 12622 | CCGCTAAGCCCGGCGCACCG | 244 |
| 12623 | CGCTAAGCCCGGCGCACCGC | 245 |
| 12624 | GCTAAGCCCGGCGCACCGCT | 246 |
| 12625 | CTAAGCCCGGCGCACCGCTC | 247 |
| 12626 | TAAGCCCGGCGCACCGCTCC | 248 |
| 12627 | AAGCCCGGCGCACCGCTCCA | 249 |
| 12628 | AGCCCGGCGCACCGCTCCAA | 250 |
| 12629 | GCCCGGCGCACCGCTCCAAC | 251 |
| 12630 | CCCGGCGCACCGCTCCAACC | 252 |
| 12631 | CCGGCGCACCGCTCCAACCT | 253 |
| 12632 | CGGCGCACCGCTCCAACCTG | 254 |
| 12633 | GGCGCACCGCTCCAACCTGT | 255 |
| 12634 | GCGCACCGCTCCAACCTGTT | 256 |
| 12635 | CGCACCGCTCCAACCTGTTC | 257 |
| 12636 | GCACCGCTCCAACCTGTTCT | 258 |
| 12637 | CACCGCTCCAACCTGTTCTC | 259 |
| 12638 | ACCGCTCCAACCTGTTCTCC | 260 |
| 12639 | CCGCTCCAACCTGTTCTCCA | 261 |
| 12640 | CGCTCCAACCTGTTCTCCAC | 262 |
| 12641 | ACGCCACCGCCGGCGCCAGG | 111 |
| 12642 | GACGCCACCGCCGGCGCCAG | 110 |
| 12643 | TGACGCCACCGCCGGCGCCA | 109 |
| 12644 | GTGACGCCACCGCCGGCGCC | 108 |
| 12645 | TGTGACGCCACCGCCGGCGC | 107 |
| 12646 | TTGTGACGCCACCGCCGGCG | 106 |
| 12647 | TTTGTGACGCCACCGCCGGC | 105 |
| 12648 | TTTTGTGACGCCACCGCCGG | 104 |
| 12649 | CTTTTGTGACGCCACCGCCG | 103 |
| 12650 | CCTTTTGTGACGCCACCGCC | 102 |
| 12651 | GCCTTTTGTGACGCCACCGC | 101 |
| 12652 | CGCCTTTTGTGACGCCACCG | 100 |
| 12653 | CCGCCTTTTGTGACGCCACC | 99 |
| 12654 | CCCGCCTTTTGTGACGCCAC | 98 |
| 12655 | TCCCGCCTTTTGTGACGCCA | 97 |
| 12656 | GTCCCGCCTTTTGTGACGCC | 96 |
| 12657 | GGTCCCGCCTTTTGTGACGC | 95 |
| 12658 | TGGTCCCGCCTTTTGTGACG | 94 |
| 12659 | GTGGTCCCGCCTTTTGTGAC | 93 |
| 12660 | TGTGGTCCCGCCTTTTGTGA | 92 |
| 12661 | CTGTGGTCCCGCCTTTTGTG | 91 |
| 12662 | ACTGTGGTCCCGCCTTTTGT | 90 |
| 12663 | CACTGTGGTCCCGCCTTTTG | 89 |
| 12664 | CCACTGTGGTCCCGCCTTTT | 88 |
| 12665 | ACCACTGTGGTCCCGCCTTT | 87 |
| 12666 | CACCACTGTGGTCCCGCCTT | 86 |
| 12667 | ACACCACTGTGGTCCCGCCT | 85 |
| 12668 | GACACCACTGTGGTCCCGCC | 84 |
| 12669 | GGACACCACTGTGGTCCCGC | 83 |
| 12670 | CGGACACCACTGTGGTCCCG | 82 |
| 12671 | TCGGACACCACTGTGGTCCC | 81 |
| 12672 | CTCGGACACCACTGTGGTCC | 80 |
| 12673 | TCTCGGACACCACTGTGGTC | 79 |
| 12674 | TTCTCGGACACCACTGTGGT | 78 |
| 12675 | CTTCTCGGACACCACTGTGG | 77 |
| 12676 | ACTTCTCGGACACCACTGTG | 76 |
| 12677 | GACTTCTCGGACACCACTGT | 75 |
| 12678 | TGACTTCTCGGACACCACTG | 74 |
| 12679 | CTGACTTCTCGGACACCACT | 73 |
| 12680 | CCTGACTTCTCGGACACCAC | 72 |
| 12681 | GCCTGACTTCTCGGACACCA | 71 |
| 12682 | TGCCTGACTTCTCGGACACC | 70 |
| 12683 | GTGCCTGACTTCTCGGACAC | 69 |
| 12684 | CGTGCCTGACTTCTCGGACA | 68 |
| 12685 | ACGTGCCTGACTTCTCGGAC | 67 |
| 12686 | TACGTGCCTGACTTCTCGGA | 66 |
| 12687 | CTACGTGCCTGACTTCTCGG | 65 |
| 12688 | GCTACGTGCCTGACTTCTCG | 64 |
| 12689 | AGCTACGTGCCTGACTTCTC | 63 |
| 12690 | GAGCTACGTGCCTGACTTCT | 62 |
| 12691 | TGAGCTACGTGCCTGACTTC | 61 |
| 12692 | CTGAGCTACGTGCCTGACTT | 60 |
| 12693 | GCTGAGCTACGTGCCTGACT | 59 |
| 12694 | CGCTGAGCTACGTGCCTGAC | 58 |
| 12695 | CCGCTGAGCTACGTGCCTGA | 57 |
| 12696 | GCCGCTGAGCTACGTGCCTG | 56 |
| 12697 | CGCCGCTGAGCTACGTGCCT | 55 |
| 12698 | CCGCCGCTGAGCTACGTGCC | 54 |
| 12699 | GCCGCCGCTGAGCTACGTGC | 53 |
| 12700 | GGCCGCCGCTGAGCTACGTG | 52 |
| 12701 | CGTTCCTCCAGCAACCGCCGCTAAGCCCGGCG | 227 |
| 12702 | GTTCCTCCAGCAACCGCCGC | 228 |
| 12703 | TTCCTCCAGCAACCGCCGCT | 229 |
| 12704 | TCCTCCAGCAACCGCCGCTA | 230 |
| 12705 | CCTCCAGCAACCGCCGCTAA | 231 |
| 12706 | CTCCAGCAACCGCCGCTAAG | 232 |
| 12707 | TCCAGCAACCGCCGCTAAGC | 233 |
| 12708 | CCAGCAACCGCCGCTAAGCC | 234 |
| 12709 | CAGCAACCGCCGCTAAGCCC | 235 |
| 12710 | AGCAACCGCCGCTAAGCCCG | 236 |
| 12711 | GCAACCGCCGCTAAGCCCGG | 237 |
| 12712 | CAACCGCCGCTAAGCCCGGC | 238 |
| 12713 | AACCGCCGCTAAGCCCGGCG | 239 |
| 12714 | ACCGCCGCTAAGCCCGGCGC | 240 |
| 12715 | CCGCCGCTAAGCCCGGCGCA | 241 |
| 12716 | CGCCGCTAAGCCCGGCGCAC | 242 |
| 12717 | GCCGCTAAGCCCGGCGCACC | 243 |
| 12718 | CCGCTAAGCCCGGCGCACCG | 244 |
| 12719 | CGCTAAGCCCGGCGCACCGC | 245 |
| 12720 | GCTAAGCCCGGCGCACCGCT | 246 |
| 12721 | CTAAGCCCGGCGCACCGCTC | 247 |
| 12722 | TAAGCCCGGCGCACCGCTCC | 248 |
| 12723 | AAGCCCGGCGCACCGCTCCA | 249 |
| 12724 | AGCCCGGCGCACCGCTCCAA | 250 |
| 12725 | GCCCGGCGCACCGCTCCAAC | 251 |
| 12726 | CCCGGCGCACCGCTCCAACC | 252 |
| 12727 | CCGGCGCACCGCTCCAACCT | 253 |
| 12728 | CGGCGCACCGCTCCAACCTG | 254 |
| 12729 | GGCGCACCGCTCCAACCTGT | 255 |
| 12730 | GCGCACCGCTCCAACCTGTT | 256 |
| 12731 | CGCACCGCTCCAACCTGTTC | 257 |
| 12732 | GCACCGCTCCAACCTGTTCT | 258 |
| 12733 | CACCGCTCCAACCTGTTCTC | 259 |
| 12734 | ACCGCTCCAACCTGTTCTCC | 260 |
| 12735 | CCGCTCCAACCTGTTCTCCA | 261 |
| 12736 | CGCTCCAACCTGTTCTCCAC | 262 |
| 12737 | CCGTTCCTCCAGCAACCGCC | 226 |
| 12738 | CCCGTTCCTCCAGCAACCGC | 225 |
| 12739 | GCCCGTTCCTCCAGCAACCG | 224 |
| 12740 | CGCCCGTTCCTCCAGCAACC | 223 |
| 12741 | CCGCCCGTTCCTCCAGCAAC | 222 |
| 12742 | TCCGCCCGTTCCTCCAGCAA | 221 |
| 12743 | CTCCGCCCGTTCCTCCAGCA | 220 |
| 12744 | ACTCCGCCCGTTCCTCCAGC | 219 |
| 12745 | GACTCCGCCCGTTCCTCCAG | 218 |
| 12746 | CGACTCCGCCCGTTCCTCCA | 217 |
| 12747 | GCGACTCCGCCCGTTCCTCC | 216 |
| 12748 | GGCGACTCCGCCCGTTCCTC | 215 |
| 12749 | GGGCGACTCCGCCCGTTCCT | 214 |
| 12750 | TGGGCGACTCCGCCCGTTCC | 213 |
| 12751 | CTGGGCGACTCCGCCCGTTC | 212 |
| 12752 | CCTGGGCGACTCCGCCCGTT | 211 |
| 12753 | CCCTGGGCGACTCCGCCCGT | 210 |
| 12754 | ACCCTGGGCGACTCCGCCCG | 209 |
| 12755 | GACCCTGGGCGACTCCGCCC | 208 |
| 12756 | GGACCCTGGGCGACTCCGCC | 207 |
| 12757 | AGGACCCTGGGCGACTCCGC | 206 |
| 12758 | CAGGACCCTGGGCGACTCCG | 205 |
| 12759 | GCAGGACCCTGGGCGACTCC | 204 |
| 12760 | GGCAGGACCCTGGGCGACTC | 203 |
| 12761 | GGGCAGGACCCTGGGCGACT | 202 |
| 12762 | AGGGCAGGACCCTGGGCGAC | 201 |
| 12763 | CAGGGCAGGACCCTGGGCGA | 200 |
| 12764 | GCAGGGCAGGACCCTGGGCG | 199 |
| 12765 | CGCAGGGCAGGACCCTGGGC | 198 |
| 12766 | CCGCAGGGCAGGACCCTGGG | 197 |
| 12767 | CCCGCAGGGCAGGACCCTGG | 196 |
| 12768 | CCCCGCAGGGCAGGACCCTG | 195 |
| 12769 | CCCCCGCAGGGCAGGACCCT | 194 |
| 12770 | ACCCCCGCAGGGCAGGACCC | 193 |
| 12771 | GACCCCCGCAGGGCAGGACC | 192 |
| 12772 | CGACCCCCGCAGGGCAGGAC | 191 |
| 12773 | TCGACCCCCGCAGGGCAGGA | 190 |
| 12774 | CTCGACCCCCGCAGGGCAGG | 189 |
| 12775 | GCTCGACCCCCGCAGGGCAG | 188 |
| 12776 | GGCTCGACCCCCGCAGGGCA | 187 |
| 12777 | CGGCTCGACCCCCGCAGGGC | 186 |
| 12778 | TCGGCTCGACCCCCGCAGGG | 185 |
| 12779 | CTCGGCTCGACCCCCGCAGG | 184 |
| 12780 | CCTCGGCTCGACCCCCGCAG | 183 |
| 12781 | GCCTCGGCTCGACCCCCGCA | 182 |
| 12782 | TGCCTCGGCTCGACCCCCGC | 181 |
| 12783 | CTGCCTCGGCTCGACCCCCG | 180 |
| 12784 | CCTGCCTCGGCTCGACCCCC | 179 |
| 12785 | GCCTGCCTCGGCTCGACCCC | 178 |
| 12786 | CGCCTGCCTCGGCTCGACCC | 177 |
| 12787 | CCGCCTGCCTCGGCTCGACC | 176 |
| 12788 | ACCGCCTGCCTCGGCTCGAC | 175 |
| 12789 | CACCGCCTGCCTCGGCTCGA | 174 |
| 12790 | TCACCGCCTGCCTCGGCTCG | 173 |
| 12791 | GTCACCGCCTGCCTCGGCTC | 172 |
| 12792 | AGTCACCGCCTGCCTCGGCT | 171 |
| 12793 | AAGTCACCGCCTGCCTCGGC | 170 |
| 12794 | GAAGTCACCGCCTGCCTCGG | 169 |
| 12795 | GGAAGTCACCGCCTGCCTCG | 168 |
| 12796 | GGGAAGTCACCGCCTGCCTC | 167 |
| 12797 | GGGGAAGTCACCGCCTGCCT | 166 |
| 12798 | TGGGGAAGTCACCGCCTGCC | 165 |
| 12799 | GTGGGGAAGTCACCGCCTGC | 164 |
| 12800 | AGTGGGGAAGTCACCGCCTG | 163 |
| 12801 | GAGTGGGGAAGTCACCGCCT | 162 |
| 12802 | CGAGTGGGGAAGTCACCGCC | 161 |
| 12803 | CCGAGTGGGGAAGTCACCGC | 160 |
| 12804 | CCCGAGTGGGGAAGTCACCG | 159 |
| 12805 | CCCCGAGTGGGGAAGTCACC | 158 |
| 12806 | GCCCCGAGTGGGGAAGTCAC | 157 |
| 12807 | CGCCCCGAGTGGGGAAGTCA | 156 |
| 12808 | CCGCCCCGAGTGGGGAAGTC | 155 |
| 12809 | TCCGCCCCGAGTGGGGAAGT | 154 |
| 12810 | CTCCGCCCCGAGTGGGGAAG | 153 |
| 12811 | GCTCCGCCCCGAGTGGGGAA | 152 |
| 12812 | GGCTCCGCCCCGAGTGGGGA | 151 |
| 12813 | CGGCTCCGCCCCGAGTGGGG | 150 |
| 12814 | GCGGCTCCGCCCCGAGTGGG | 149 |
| 12815 | TGCGGCTCCGCCCCGAGTGG | 148 |
| 12816 | CTGCGGCTCCGCCCCGAGTG | 147 |
| 12817 | GCTGCGGCTCCGCCCCGAGT | 146 |
| 12818 | GGCTGCGGCTCCGCCCCGAG | 145 |
| 12819 | AGGCTGCGGCTCCGCCCCGA | 144 |
| 12820 | GAGGCTGCGGCTCCGCCCCG | 143 |
| 12821 | CGAGGCTGCGGCTCCGCCCC | 142 |
| 12822 | GCGAGGCTGCGGCTCCGCCC | 141 |
| 12823 | CGCGAGGCTGCGGCTCCGCC | 140 |
| 12824 | CCGCGAGGCTGCGGCTCCGC | 139 |
| 12825 | CCCGCGAGGCTGCGGCTCCG | 138 |
| 12826 | CCCCGCGAGGCTGCGGCTCC | 137 |
| 12827 | CCCCCGCGAGGCTGCGGCTC | 136 |
| 12828 | GCCCCCGCGAGGCTGCGGCT | 135 |
| 12829 | CGCCCCCGCGAGGCTGCGGC | 134 |
| 12830 | CCGCCCCCGCGAGGCTGCGG | 133 |
| 12831 | CCCGCCCCCGCGAGGCTGCG | 132 |
| 12832 | CCCCGCCCCCGCGAGGCTGC | 131 |
| 12833 | GCCCCGCCCCCGCGAGGCTG | 130 |
| 12834 | GGCCCCGCCCCCGCGAGGCT | 129 |
| 12835 | AGGCCCCGCCCCCGCGAGGC | 128 |
| 12836 | CAGGCCCCGCCCCCGCGAGG | 127 |
| 12837 | CCAGGCCCCGCCCCCGCGAG | 126 |
| 12838 | GCCAGGCCCCGCCCCCGCGA | 125 |
| 12839 | CGCCAGGCCCCGCCCCCGCG | 124 |
| 12840 | GCGCCAGGCCCCGCCCCCGC | 123 |
| 12841 | GGCGCCAGGCCCCGCCCCCG | 122 |
| 12842 | CGGCGCCAGGCCCCGCCCCC | 121 |
| 12843 | CCGGCGCCAGGCCCCGCCCC | 120 |
| 12844 | GCCGGCGCCAGGCCCCGCCC | 119 |
| 12845 | CGCCGGCGCCAGGCCCCGCC | 118 |
| 12846 | CCGCCGGCGCCAGGCCCCGC | 117 |
| 12847 | ACCGCCGGCGCCAGGCCCCG | 116 |
| 12848 | CACCGCCGGCGCCAGGCCCC | 115 |
| 12849 | CCACCGCCGGCGCCAGGCCC | 114 |
| 12850 | GCCACCGCCGGCGCCAGGCC | 113 |
| 12851 | CGCCACCGCCGGCGCCAGGC | 112 |
| 12852 | ACGCCACCGCCGGCGCCAGG | 111 |
| 12853 | GACGCCACCGCCGGCGCCAG | 110 |
| 12854 | TGACGCCACCGCCGGCGCCA | 109 |
| 12855 | GTGACGCCACCGCCGGCGCC | 108 |
| 12856 | TGTGACGCCACCGCCGGCGC | 107 |
| 12857 | TTGTGACGCCACCGCCGGCG | 106 |
| 12858 | TTTGTGACGCCACCGCCGGC | 105 |
| 12859 | TTTTGTGACGCCACCGCCGG | 104 |
| 12860 | CTTTTGTGACGCCACCGCCG | 103 |
| 12861 | CCTTTTGTGACGCCACCGCC | 102 |
| 12862 | GCCTTTTGTGACGCCACCGC | 101 |
| 12863 | CGCCTTTTGTGACGCCACCG | 100 |
| 12864 | CCGCCTTTTGTGACGCCACC | 99 |
| 12865 | CCCGCCTTTTGTGACGCCAC | 98 |
| 12866 | TCCCGCCTTTTGTGACGCCA | 97 |
| 12867 | GTCCCGCCTTTTGTGACGCC | 96 |
| 12868 | GGTCCCGCCTTTTGTGACGC | 95 |
| 12869 | TGGTCCCGCCTTTTGTGACG | 94 |
| 12870 | GTGGTCCCGCCTTTTGTGAC | 93 |
| 12871 | TGTGGTCCCGCCTTTTGTGA | 92 |
| 12872 | CTGTGGTCCCGCCTTTTGTG | 91 |
| 12873 | ACTGTGGTCCCGCCTTTTGT | 90 |
| 12874 | CACTGTGGTCCCGCCTTTTG | 89 |
| 12875 | CCACTGTGGTCCCGCCTTTT | 88 |
| 12876 | ACCACTGTGGTCCCGCCTTT | 87 |
| 12877 | CACCACTGTGGTCCCGCCTT | 86 |
| 12878 | ACACCACTGTGGTCCCGCCT | 85 |
| 12879 | GACACCACTGTGGTCCCGCC | 84 |
| 12880 | GGACACCACTGTGGTCCCGC | 83 |
| 12881 | CGGACACCACTGTGGTCCCG | 82 |
| 12882 | TCGGACACCACTGTGGTCCC | 81 |
| 12883 | CTCGGACACCACTGTGGTCC | 80 |
| 12884 | TCTCGGACACCACTGTGGTC | 79 |
| 12885 | TTCTCGGACACCACTGTGGT | 78 |
| 12886 | CTTCTCGGACACCACTGTGG | 77 |
| 12887 | ACTTCTCGGACACCACTGTG | 76 |
| 12888 | GACTTCTCGGACACCACTGT | 75 |
| 12889 | TGACTTCTCGGACACCACTG | 74 |
| 12890 | CTGACTTCTCGGACACCACT | 73 |
| 12891 | CCTGACTTCTCGGACACCAC | 72 |
| 12892 | GCCTGACTTCTCGGACACCA | 71 |
| 12893 | TGCCTGACTTCTCGGACACC | 70 |
| 12894 | GTGCCTGACTTCTCGGACAC | 69 |
| 12895 | CGTGCCTGACTTCTCGGACA | 68 |
| 12896 | ACGTGCCTGACTTCTCGGAC | 67 |
| 12897 | TACGTGCCTGACTTCTCGGA | 66 |
| 12898 | CTACGTGCCTGACTTCTCGG | 65 |
| 12899 | GCTACGTGCCTGACTTCTCG | 64 |
| 12900 | AGCTACGTGCCTGACTTCTC | 63 |
| 12901 | GAGCTACGTGCCTGACTTCT | 62 |
| 12902 | TGAGCTACGTGCCTGACTTC | 61 |
| 12903 | CTGAGCTACGTGCCTGACTT | 60 |
| 12904 | GCTGAGCTACGTGCCTGACT | 59 |
| 12905 | CGCTGAGCTACGTGCCTGAC | 58 |
| 12906 | CCGCTGAGCTACGTGCCTGA | 57 |
| 12907 | GCCGCTGAGCTACGTGCCTG | 56 |
| 12908 | CGCCGCTGAGCTACGTGCCT | 55 |
| 12909 | CCGCCGCTGAGCTACGTGCC | 54 |
| 12910 | GCCGCCGCTGAGCTACGTGC | 53 |
| 12911 | GGCCGCCGCTGAGCTACGTG | 52 |
| 12912 | CGCCTGCCTCGGCTCGACCCCCGCAG | 177 |
| 12913 | GCCTGCCTCGGCTCGACCCC | 178 |
| 12914 | CCTGCCTCGGCTCGACCCCC | 179 |
| 12915 | CTGCCTCGGCTCGACCCCCG | 180 |
| 12916 | TGCCTCGGCTCGACCCCCGC | 181 |
| 12917 | GCCTCGGCTCGACCCCCGCA | 182 |
| 12918 | CCTCGGCTCGACCCCCGCAG | 183 |
| 12919 | CTCGGCTCGACCCCCGCAGG | 184 |
| 12920 | TCGGCTCGACCCCCGCAGGG | 185 |
| 12921 | CGGCTCGACCCCCGCAGGGC | 186 |
| 12922 | GGCTCGACCCCCGCAGGGCA | 187 |
| 12923 | GCTCGACCCCCGCAGGGCAG | 188 |
| 12924 | CTCGACCCCCGCAGGGCAGG | 189 |
| 12925 | TCGACCCCCGCAGGGCAGGA | 190 |
| 12926 | CGACCCCCGCAGGGCAGGAC | 191 |
| 12927 | GACCCCCGCAGGGCAGGACC | 192 |
| 12928 | ACCCCCGCAGGGCAGGACCC | 193 |
| 12929 | CCCCCGCAGGGCAGGACCCT | 194 |
| 12930 | CCCCGCAGGGCAGGACCCTG | 195 |
| 12931 | CCCGCAGGGCAGGACCCTGG | 196 |
| 12932 | CCGCAGGGCAGGACCCTGGG | 197 |
| 12933 | CGCAGGGCAGGACCCTGGGC | 198 |
| 12934 | GCAGGGCAGGACCCTGGGCG | 199 |
| 12935 | CAGGGCAGGACCCTGGGCGA | 200 |
| 12936 | AGGGCAGGACCCTGGGCGAC | 201 |
| 12937 | GGGCAGGACCCTGGGCGACT | 202 |
| 12938 | GGCAGGACCCTGGGCGACTC | 203 |
| 12939 | GCAGGACCCTGGGCGACTCC | 204 |
| 12940 | CAGGACCCTGGGCGACTCCG | 205 |
| 12941 | AGGACCCTGGGCGACTCCGC | 206 |
| 12942 | GGACCCTGGGCGACTCCGCC | 207 |
| 12943 | GACCCTGGGCGACTCCGCCC | 208 |
| 12944 | ACCCTGGGCGACTCCGCCCG | 209 |
| 12945 | CCCTGGGCGACTCCGCCCGT | 210 |
| 12946 | CCTGGGCGACTCCGCCCGTT | 211 |
| 12947 | CTGGGCGACTCCGCCCGTTC | 212 |
| 12948 | TGGGCGACTCCGCCCGTTCC | 213 |
| 12949 | GGGCGACTCCGCCCGTTCCT | 214 |
| 12950 | GGCGACTCCGCCCGTTCCTC | 215 |
| 12951 | GCGACTCCGCCCGTTCCTCC | 216 |
| 12952 | CGACTCCGCCCGTTCCTCCA | 217 |
| 12953 | GACTCCGCCCGTTCCTCCAG | 218 |
| 12954 | ACTCCGCCCGTTCCTCCAGC | 219 |
| 12955 | CTCCGCCCGTTCCTCCAGCA | 220 |
| 12956 | TCCGCCCGTTCCTCCAGCAA | 221 |
| 12957 | CCGCCCGTTCCTCCAGCAAC | 222 |
| 12958 | CGCCCGTTCCTCCAGCAACC | 223 |
| 12959 | GCCCGTTCCTCCAGCAACCG | 224 |
| 12960 | CCCGTTCCTCCAGCAACCGC | 225 |
| 12961 | CCGTTCCTCCAGCAACCGCC | 226 |
| 12962 | CGTTCCTCCAGCAACCGCCG | 227 |
| 12963 | GTTCCTCCAGCAACCGCCGC | 228 |
| 12964 | TTCCTCCAGCAACCGCCGCT | 229 |
| 12965 | TCCTCCAGCAACCGCCGCTA | 230 |
| 12966 | CCTCCAGCAACCGCCGCTAA | 231 |
| 12967 | CTCCAGCAACCGCCGCTAAG | 232 |
| 12968 | TCCAGCAACCGCCGCTAAGC | 233 |
| 12969 | CCAGCAACCGCCGCTAAGCC | 234 |
| 12970 | CAGCAACCGCCGCTAAGCCC | 235 |
| 12971 | AGCAACCGCCGCTAAGCCCG | 236 |
| 12972 | GCAACCGCCGCTAAGCCCGG | 237 |
| 12973 | CAACCGCCGCTAAGCCCGGC | 238 |
| 12974 | AACCGCCGCTAAGCCCGGCG | 239 |
| 12975 | ACCGCCGCTAAGCCCGGCGC | 240 |
| 12976 | CCGCCGCTAAGCCCGGCGCA | 241 |
| 12977 | CGCCGCTAAGCCCGGCGCAC | 242 |
| 12978 | GCCGCTAAGCCCGGCGCACC | 243 |
| 12979 | CCGCTAAGCCCGGCGCACCG | 244 |
| 12980 | CGCTAAGCCCGGCGCACCGC | 245 |
| 12981 | GCTAAGCCCGGCGCACCGCT | 246 |
| 12982 | CTAAGCCCGGCGCACCGCTC | 247 |
| 12983 | TAAGCCCGGCGCACCGCTCC | 248 |
| 12984 | AAGCCCGGCGCACCGCTCCA | 249 |
| 12985 | AGCCCGGCGCACCGCTCCAA | 250 |
| 12986 | GCCCGGCGCACCGCTCCAAC | 251 |
| 12987 | CCCGGCGCACCGCTCCAACC | 252 |
| 12988 | CCGGCGCACCGCTCCAACCT | 253 |
| 12989 | CGGCGCACCGCTCCAACCTG | 254 |
| 12990 | GGCGCACCGCTCCAACCTGT | 255 |
| 12991 | GCGCACCGCTCCAACCTGTT | 256 |
| 12992 | CGCACCGCTCCAACCTGTTC | 257 |
| 12993 | GCACCGCTCCAACCTGTTCT | 258 |
| 12994 | CACCGCTCCAACCTGTTCTC | 259 |
| 12995 | ACCGCTCCAACCTGTTCTCC | 260 |
| 12996 | CCGCTCCAACCTGTTCTCCA | 261 |
| 12997 | CGCTCCAACCTGTTCTCCAC | 262 |
| 12998 | CCGCCTGCCTCGGCTCGACC | 176 |
| 12999 | ACCGCCTGCCTCGGCTCGAC | 175 |
| 13000 | CACCGCCTGCCTCGGCTCGA | 174 |
| 13001 | TCACCGCCTGCCTCGGCTCG | 173 |
| 13002 | GTCACCGCCTGCCTCGGCTC | 172 |
| 13003 | AGTCACCGCCTGCCTCGGCT | 171 |
| 13004 | AAGTCACCGCCTGCCTCGGC | 170 |
| 13005 | GAAGTCACCGCCTGCCTCGG | 169 |
| 13006 | GGAAGTCACCGCCTGCCTCG | 168 |
| 13007 | GGGAAGTCACCGCCTGCCTC | 167 |
| 13008 | GGGGAAGTCACCGCCTGCCT | 166 |
| 13009 | TGGGGAAGTCACCGCCTGCC | 165 |
| 13010 | GTGGGGAAGTCACCGCCTGC | 164 |
| 13011 | AGTGGGGAAGTCACCGCCTG | 163 |
| 13012 | GAGTGGGGAAGTCACCGCCT | 162 |
| 13013 | CGAGTGGGGAAGTCACCGCC | 161 |
| 13014 | CCGAGTGGGGAAGTCACCGC | 160 |
| 13015 | CCCGAGTGGGGAAGTCACCG | 159 |
| 13016 | CCCCGAGTGGGGAAGTCACC | 158 |
| 13017 | GCCCCGAGTGGGGAAGTCAC | 157 |
| 13018 | CGCCCCGAGTGGGGAAGTCA | 156 |
| 13019 | CCGCCCCGAGTGGGGAAGTC | 155 |
| 13020 | TCCGCCCCGAGTGGGGAAGT | 154 |
| 13021 | CTCCGCCCCGAGTGGGGAAG | 153 |
| 13022 | GCTCCGCCCCGAGTGGGGAA | 152 |
| 13023 | GGCTCCGCCCCGAGTGGGGA | 151 |
| 13024 | CGGCTCCGCCCCGAGTGGGG | 150 |
| 13025 | GCGGCTCCGCCCCGAGTGGG | 149 |
| 13026 | TGCGGCTCCGCCCCGAGTGG | 148 |
| 13027 | CTGCGGCTCCGCCCCGAGTG | 147 |
| 13028 | GCTGCGGCTCCGCCCCGAGT | 146 |
| 13029 | GGCTGCGGCTCCGCCCCGAG | 145 |
| 13030 | AGGCTGCGGCTCCGCCCCGA | 144 |
| 13031 | GAGGCTGCGGCTCCGCCCCG | 143 |
| 13032 | CGAGGCTGCGGCTCCGCCCC | 142 |
| 13033 | GCGAGGCTGCGGCTCCGCCC | 141 |
| 13034 | CGCGAGGCTGCGGCTCCGCC | 140 |
| 13035 | CCGCGAGGCTGCGGCTCCGC | 139 |
| 13036 | CCCGCGAGGCTGCGGCTCCG | 138 |
| 13037 | CCCCGCGAGGCTGCGGCTCC | 137 |
| 13038 | CCCCCGCGAGGCTGCGGCTC | 136 |
| 13039 | GCCCCCGCGAGGCTGCGGCT | 135 |
| 13040 | CGCCCCCGCGAGGCTGCGGC | 134 |
| 13041 | CCGCCCCCGCGAGGCTGCGG | 133 |
| 13042 | CCCGCCCCCGCGAGGCTGCG | 132 |
| 13043 | CCCCGCCCCCGCGAGGCTGC | 131 |
| 13044 | GCCCCGCCCCCGCGAGGCTG | 130 |
| 13045 | GGCCCCGCCCCCGCGAGGCT | 129 |
| 13046 | AGGCCCCGCCCCCGCGAGGC | 128 |
| 13047 | CAGGCCCCGCCCCCGCGAGG | 127 |
| 13048 | CCAGGCCCCGCCCCCGCGAG | 126 |
| 13049 | GCCAGGCCCCGCCCCCGCGA | 125 |
| 13050 | CGCCAGGCCCCGCCCCCGCG | 124 |
| 13051 | GCGCCAGGCCCCGCCCCCGC | 123 |
| 13052 | GGCGCCAGGCCCCGCCCCCG | 122 |
| 13053 | CGGCGCCAGGCCCCGCCCCC | 121 |
| 13054 | CCGGCGCCAGGCCCCGCCCC | 120 |
| 13055 | GCCGGCGCCAGGCCCCGCCC | 119 |
| 13056 | CGCCGGCGCCAGGCCCCGCC | 118 |
| 13057 | CCGCCGGCGCCAGGCCCCGC | 117 |
| 13058 | ACCGCCGGCGCCAGGCCCCG | 116 |
| 13059 | CACCGCCGGCGCCAGGCCCC | 115 |
| 13060 | CCACCGCCGGCGCCAGGCCC | 114 |
| 13061 | GCCACCGCCGGCGCCAGGCC | 113 |
| 13062 | CGCCACCGCCGGCGCCAGGC | 112 |
| 13063 | ACGCCACCGCCGGCGCCAGG | 111 |
| 13064 | GACGCCACCGCCGGCGCCAG | 110 |
| 13065 | TGACGCCACCGCCGGCGCCA | 109 |
| 13066 | GTGACGCCACCGCCGGCGCC | 108 |
| 13067 | TGTGACGCCACCGCCGGCGC | 107 |
| 13068 | TTGTGACGCCACCGCCGGCG | 106 |
| 13069 | TTTGTGACGCCACCGCCGGC | 105 |
| 13070 | TTTTGTGACGCCACCGCCGG | 104 |
| 13071 | CTTTTGTGACGCCACCGCCG | 103 |
| 13072 | CCTTTTGTGACGCCACCGCC | 102 |
| 13073 | GCCTTTTGTGACGCCACCGC | 101 |
| 13074 | CGCCTTTTGTGACGCCACCG | 100 |
| 13075 | CCGCCTTTTGTGACGCCACC | 99 |
| 13076 | CCCGCCTTTTGTGACGCCAC | 98 |
| 13077 | TCCCGCCTTTTGTGACGCCA | 97 |
| 13078 | GTCCCGCCTTTTGTGACGCC | 96 |
| 13079 | GGTCCCGCCTTTTGTGACGC | 95 |
| 13080 | TGGTCCCGCCTTTTGTGACG | 94 |
| 13081 | GTGGTCCCGCCTTTTGTGAC | 93 |
| 13082 | TGTGGTCCCGCCTTTTGTGA | 92 |
| 13083 | CTGTGGTCCCGCCTTTTGTG | 91 |
| 13084 | ACTGTGGTCCCGCCTTTTGT | 90 |
| 13085 | CACTGTGGTCCCGCCTTTTG | 89 |
| 13086 | CCACTGTGGTCCCGCCTTTT | 88 |
| 13087 | ACCACTGTGGTCCCGCCTTT | 87 |
| 13088 | CACCACTGTGGTCCCGCCTT | 86 |
| 13089 | ACACCACTGTGGTCCCGCCT | 85 |
| 13090 | GACACCACTGTGGTCCCGCC | 84 |
| 13091 | GGACACCACTGTGGTCCCGC | 83 |
| 13092 | CGGACACCACTGTGGTCCCG | 82 |
| 13093 | TCGGACACCACTGTGGTCCC | 81 |
| 13094 | CTCGGACACCACTGTGGTCC | 80 |
| 13095 | TCTCGGACACCACTGTGGTC | 79 |
| 13096 | TTCTCGGACACCACTGTGGT | 78 |
| 13097 | CTTCTCGGACACCACTGTGG | 77 |
| 13098 | ACTTCTCGGACACCACTGTG | 76 |
| 13099 | GACTTCTCGGACACCACTGT | 75 |
| 13100 | TGACTTCTCGGACACCACTG | 74 |
| 13101 | CTGACTTCTCGGACACCACT | 73 |
| 13102 | CCTGACTTCTCGGACACCAC | 72 |
| 13103 | GCCTGACTTCTCGGACACCA | 71 |
| 13104 | TGCCTGACTTCTCGGACACC | 70 |
| 13105 | GTGCCTGACTTCTCGGACAC | 69 |
| 13106 | CGTGCCTGACTTCTCGGACA | 68 |
| 13107 | ACGTGCCTGACTTCTCGGAC | 67 |
| 13108 | TACGTGCCTGACTTCTCGGA | 66 |
| 13109 | CTACGTGCCTGACTTCTCGG | 65 |
| 13110 | GCTACGTGCCTGACTTCTCG | 64 |
| 13111 | AGCTACGTGCCTGACTTCTC | 63 |
| 13112 | GAGCTACGTGCCTGACTTCT | 62 |
| 13113 | TGAGCTACGTGCCTGACTTC | 61 |
| 13114 | CTGAGCTACGTGCCTGACTT | 60 |
| 13115 | GCTGAGCTACGTGCCTGACT | 59 |
| 13116 | CGCTGAGCTACGTGCCTGAC | 58 |
| 13117 | CCGCTGAGCTACGTGCCTGA | 57 |
| 13118 | GCCGCTGAGCTACGTGCCTG | 56 |
| 13119 | CGCCGCTGAGCTACGTGCCT | 55 |
| 13120 | CCGCCGCTGAGCTACGTGCC | 54 |
| 13121 | GCCGCCGCTGAGCTACGTGC | 53 |
| 13122 | GGCCGCCGCTGAGCTACGTG | 52 |
| 13123 | CGGCTAGAAATCGGCCTGTTCCGGCCTCGCCT | 286 |
| 13124 | GGCTAGAAATCGGCCTGTTC | 287 |
| 13125 | GCTAGAAATCGGCCTGTTCC | 288 |
| 13126 | CTAGAAATCGGCCTGTTCCG | 289 |
| 13127 | TAGAAATCGGCCTGTTCCGG | 290 |
| 13128 | AGAAATCGGCCTGTTCCGGC | 291 |
| 13129 | GAAATCGGCCTGTTCCGGCC | 292 |
| 13130 | AAATCGGCCTGTTCCGGCCT | 293 |
| 13131 | AATCGGCCTGTTCCGGCCTC | 294 |
| 13132 | ATCGGCCTGTTCCGGCCTCG | 295 |
| 13133 | TCGGCCTGTTCCGGCCTCGC | 296 |
| 13134 | CGGCCTGTTCCGGCCTCGCC | 297 |
| 13135 | GGCCTGTTCCGGCCTCGCCT | 298 |
| 13136 | GCCTGTTCCGGCCTCGCCTC | 299 |
| 13137 | CCTGTTCCGGCCTCGCCTCG | 300 |
| 13138 | CTGTTCCGGCCTCGCCTCGG | 301 |
| 13139 | TGTTCCGGCCTCGCCTCGGG | 302 |
| 13140 | GTTCCGGCCTCGCCTCGGGT | 303 |
| 13141 | TTCCGGCCTCGCCTCGGGTC | 304 |
| 13142 | TCCGGCCTCGCCTCGGGTCT | 305 |
| 13143 | CCGGCCTCGCCTCGGGTCTT | 306 |
| 13144 | CGGCCTCGCCTCGGGTCTTT | 307 |
| 13145 | GGCCTCGCCTCGGGTCTTTC | 308 |
| 13146 | GCCTCGCCTCGGGTCTTTCT | 309 |
| 13147 | CCTCGCCTCGGGTCTTTCTT | 310 |
| 13148 | CTCGCCTCGGGTCTTTCTTA | 311 |
| 13149 | TCGCCTCGGGTCTTTCTTAG | 312 |
| 13150 | CGCCTCGGGTCTTTCTTAGT | 313 |
| 13151 | GCCTCGGGTCTTTCTTAGTC | 314 |
| 13152 | CCTCGGGTCTTTCTTAGTCC | 315 |
| 13153 | CTCGGGTCTTTCTTAGTCCT | 316 |
| 13154 | TCGGGTCTTTCTTAGTCCTT | 317 |
| 13155 | CGGGTCTTTCTTAGTCCTTT | 318 |
| 13156 | GCGGCTAGAAATCGGCCTGT | 285 |
| 13157 | GGCGGCTAGAAATCGGCCTG | 284 |
| 13158 | TGGCGGCTAGAAATCGGCCT | 283 |
| 13159 | TTGGCGGCTAGAAATCGGCC | 282 |
| 13160 | CTTGGCGGCTAGAAATCGGC | 281 |
| 13161 | ACTTGGCGGCTAGAAATCGG | 280 |
| 13162 | CACTTGGCGGCTAGAAATCG | 279 |
| 13163 | CCACTTGGCGGCTAGAAATC | 278 |
| 13164 | TCCACTTGGCGGCTAGAAAT | 277 |
| 13165 | CTCCACTTGGCGGCTAGAAA | 276 |
| 13166 | TCTCCACTTGGCGGCTAGAA | 275 |
| 13167 | TTCTCCACTTGGCGGCTAGA | 274 |
| 13168 | GTTCTCCACTTGGCGGCTAG | 273 |
| 13169 | TGTTCTCCACTTGGCGGCTA | 272 |
| 13170 | CTGTTCTCCACTTGGCGGCT | 271 |
| 13171 | CCTGTTCTCCACTTGGCGGC | 270 |
| 13172 | ACCTGTTCTCCACTTGGCGG | 269 |
| 13173 | AACCTGTTCTCCACTTGGCG | 268 |
| 13174 | CGGGGGTGGGGATGCGGCGGTGAACCCG | 377 |
| 13175 | CGCGGCAGGTGAGAGGGGAGCTGCCCCTGCG | 558 |
| 13176 | CGCGTGCACGTGTGTCCACATGAGTGC | 3650 |
| 13177 | GCGTGCACGTGTGTCCACAT | 3651 |
| 13178 | CGTGCACGTGTGTCCACATG | 3652 |
| 13179 | GTGCACGTGTGTCCACATGA | 3653 |
| 13180 | TGCACGTGTGTCCACATGAG | 3654 |
| 13181 | GCACGTGTGTCCACATGAGT | 3655 |
| 13182 | CACGTGTGTCCACATGAGTG | 3656 |
| 13183 | ACGTGTGTCCACATGAGTGC | 3657 |
| 13184 | CGTGTGTCCACATGAGTGCT | 3658 |
| 13185 | GCGCGTGCACGTGTGTCCAC | 3649 |
| 13186 | TGCGCGTGCACGTGTGTCCA | 3648 |
| 13187 | GTGCGCGTGCACGTGTGTCC | 3647 |
| 13188 | TGTGCGCGTGCACGTGTGTC | 3646 |
| 13189 | GTGTGCGCGTGCACGTGTGT | 3645 |
| 13190 | TGTGTGCGCGTGCACGTGTG | 3644 |
| 13191 | GTGTGTGCGCGTGCACGTGT | 3643 |
| 13192 | TGTGTGTGCGCGTGCACGTG | 3642 |
| 13193 | ATGTGTGTGCGCGTGCACGT | 3641 |
| 13194 | CATGTGTGTGCGCGTGCACG | 3640 |
| 13195 | CCATGTGTGTGCGCGTGCAC | 3639 |
| 13196 | TCCATGTGTGTGCGCGTGCA | 3638 |
| 13197 | GTCCATGTGTGTGCGCGTGC | 3637 |
| 13198 | TGTCCATGTGTGTGCGCGTG | 3636 |
| 13199 | GTGTCCATGTGTGTGCGCGT | 3635 |
| 13200 | TGTGTCCATGTGTGTGCGCG | 3634 |
| 13201 | GTGTGTCCATGTGTGTGCGC | 3633 |
| 13202 | TGTGTGTCCATGTGTGTGCG | 3632 |
| 13203 | CGCCACCGCCGGCGCCAGGCCCCGCC | 112 |
| 13204 | GCCACCGCCGGCGCCAGGCC | 113 |
| 13205 | CCACCGCCGGCGCCAGGCCC | 114 |
| 13206 | CACCGCCGGCGCCAGGCCCC | 115 |
| 13207 | ACCGCCGGCGCCAGGCCCCG | 116 |
| 13208 | CCGCCGGCGCCAGGCCCCGC | 117 |
| 13209 | CGCCGGCGCCAGGCCCCGCC | 118 |
| 13210 | GCCGGCGCCAGGCCCCGCCC | 119 |
| 13211 | CCGGCGCCAGGCCCCGCCCC | 120 |
| 13212 | CGGCGCCAGGCCCCGCCCCC | 121 |
| 13213 | GGCGCCAGGCCCCGCCCCCG | 122 |
| 13214 | GCGCCAGGCCCCGCCCCCGC | 123 |
| 13215 | CGCCAGGCCCCGCCCCCGCG | 124 |
| 13216 | GCCAGGCCCCGCCCCCGCGA | 125 |
| 13217 | CCAGGCCCCGCCCCCGCGAG | 126 |
| 13218 | CAGGCCCCGCCCCCGCGAGG | 127 |
| 13219 | AGGCCCCGCCCCCGCGAGGC | 128 |
| 13220 | GGCCCCGCCCCCGCGAGGCT | 129 |
| 13221 | GCCCCGCCCCCGCGAGGCTG | 130 |
| 13222 | CCCCGCCCCCGCGAGGCTGC | 131 |
| 13223 | CCCGCCCCCGCGAGGCTGCG | 132 |
| 13224 | CCGCCCCCGCGAGGCTGCGG | 133 |
| 13225 | CGCCCCCGCGAGGCTGCGGC | 134 |
| 13226 | GCCCCCGCGAGGCTGCGGCT | 135 |
| 13227 | CCCCCGCGAGGCTGCGGCTC | 136 |
| 13228 | CCCCGCGAGGCTGCGGCTCC | 137 |
| 13229 | CCCGCGAGGCTGCGGCTCCG | 138 |
| 13230 | CCGCGAGGCTGCGGCTCCGC | 139 |
| 13231 | CGCGAGGCTGCGGCTCCGCC | 140 |
| 13232 | GCGAGGCTGCGGCTCCGCCC | 141 |
| 13233 | CGAGGCTGCGGCTCCGCCCC | 142 |
| 13234 | GAGGCTGCGGCTCCGCCCCG | 143 |
| 13235 | AGGCTGCGGCTCCGCCCCGA | 144 |
| 13236 | GGCTGCGGCTCCGCCCCGAG | 145 |
| 13237 | GCTGCGGCTCCGCCCCGAGT | 146 |
| 13238 | CTGCGGCTCCGCCCCGAGTG | 147 |
| 13239 | TGCGGCTCCGCCCCGAGTGG | 148 |
| 13240 | GCGGCTCCGCCCCGAGTGGG | 149 |
| 13241 | CGGCTCCGCCCCGAGTGGGG | 150 |
| 13242 | GGCTCCGCCCCGAGTGGGGA | 151 |
| 13243 | GCTCCGCCCCGAGTGGGGAA | 152 |
| 13244 | CTCCGCCCCGAGTGGGGAAG | 153 |
| 13245 | TCCGCCCCGAGTGGGGAAGT | 154 |
| 13246 | CCGCCCCGAGTGGGGAAGTC | 155 |
| 13247 | CGCCCCGAGTGGGGAAGTCA | 156 |
| 13248 | GCCCCGAGTGGGGAAGTCAC | 157 |
| 13249 | CCCCGAGTGGGGAAGTCACC | 158 |
| 13250 | CCCGAGTGGGGAAGTCACCG | 159 |
| 13251 | CCGAGTGGGGAAGTCACCGC | 160 |
| 13252 | CGAGTGGGGAAGTCACCGCC | 161 |
| 13253 | GAGTGGGGAAGTCACCGCCT | 162 |
| 13254 | AGTGGGGAAGTCACCGCCTG | 163 |
| 13255 | GTGGGGAAGTCACCGCCTGC | 164 |
| 13256 | TGGGGAAGTCACCGCCTGCC | 165 |
| 13257 | GGGGAAGTCACCGCCTGCCT | 166 |
| 13258 | GGGAAGTCACCGCCTGCCTC | 167 |
| 13259 | GGAAGTCACCGCCTGCCTCG | 168 |
| 13260 | GAAGTCACCGCCTGCCTCGG | 169 |
| 13261 | AAGTCACCGCCTGCCTCGGC | 170 |
| 13262 | AGTCACCGCCTGCCTCGGCT | 171 |
| 13263 | GTCACCGCCTGCCTCGGCTC | 172 |
| 13264 | TCACCGCCTGCCTCGGCTCG | 173 |
| 13265 | CACCGCCTGCCTCGGCTCGA | 174 |
| 13266 | ACCGCCTGCCTCGGCTCGAC | 175 |
| 13267 | CCGCCTGCCTCGGCTCGACC | 176 |
| 13268 | CGCCTGCCTCGGCTCGACCC | 177 |
| 13269 | GCCTGCCTCGGCTCGACCCC | 178 |
| 13270 | CCTGCCTCGGCTCGACCCCC | 179 |
| 13271 | CTGCCTCGGCTCGACCCCCG | 180 |
| 13272 | TGCCTCGGCTCGACCCCCGC | 181 |
| 13273 | GCCTCGGCTCGACCCCCGCA | 182 |
| 13274 | CCTCGGCTCGACCCCCGCAG | 183 |
| 13275 | CTCGGCTCGACCCCCGCAGG | 184 |
| 13276 | TCGGCTCGACCCCCGCAGGG | 185 |
| 13277 | CGGCTCGACCCCCGCAGGGC | 186 |
| 13278 | GGCTCGACCCCCGCAGGGCA | 187 |
| 13279 | GCTCGACCCCCGCAGGGCAG | 188 |
| 13280 | CTCGACCCCCGCAGGGCAGG | 189 |
| 13281 | TCGACCCCCGCAGGGCAGGA | 190 |
| 13282 | CGACCCCCGCAGGGCAGGAC | 191 |
| 13283 | GACCCCCGCAGGGCAGGACC | 192 |
| 13284 | ACCCCCGCAGGGCAGGACCC | 193 |
| 13285 | CCCCCGCAGGGCAGGACCCT | 194 |
| 13286 | CCCCGCAGGGCAGGACCCTG | 195 |
| 13287 | CCCGCAGGGCAGGACCCTGG | 196 |
| 13288 | CCGCAGGGCAGGACCCTGGG | 197 |
| 13289 | CGCAGGGCAGGACCCTGGGC | 198 |
| 13290 | GCAGGGCAGGACCCTGGGCG | 199 |
| 13291 | CAGGGCAGGACCCTGGGCGA | 200 |
| 13292 | AGGGCAGGACCCTGGGCGAC | 201 |
| 13293 | GGGCAGGACCCTGGGCGACT | 202 |
| 13294 | GGCAGGACCCTGGGCGACTC | 203 |
| 13295 | GCAGGACCCTGGGCGACTCC | 204 |
| 13296 | CAGGACCCTGGGCGACTCCG | 205 |
| 13297 | AGGACCCTGGGCGACTCCGC | 206 |
| 13298 | GGACCCTGGGCGACTCCGCC | 207 |
| 13299 | GACCCTGGGCGACTCCGCCC | 208 |
| 13300 | ACCCTGGGCGACTCCGCCCG | 209 |
| 13301 | CCCTGGGCGACTCCGCCCGT | 210 |
| 13302 | CCTGGGCGACTCCGCCCGTT | 211 |
| 13303 | CTGGGCGACTCCGCCCGTTC | 212 |
| 13304 | TGGGCGACTCCGCCCGTTCC | 213 |
| 13305 | GGGCGACTCCGCCCGTTCCT | 214 |
| 13306 | GGCGACTCCGCCCGTTCCTC | 215 |
| 13307 | GCGACTCCGCCCGTTCCTCC | 216 |
| 13308 | CGACTCCGCCCGTTCCTCCA | 217 |
| 13309 | GACTCCGCCCGTTCCTCCAG | 218 |
| 13310 | ACTCCGCCCGTTCCTCCAGC | 219 |
| 13311 | CTCCGCCCGTTCCTCCAGCA | 220 |
| 13312 | TCCGCCCGTTCCTCCAGCAA | 221 |
| 13313 | CCGCCCGTTCCTCCAGCAAC | 222 |
| 13314 | CGCCCGTTCCTCCAGCAACC | 223 |
| 13315 | GCCCGTTCCTCCAGCAACCG | 224 |
| 13316 | CCCGTTCCTCCAGCAACCGC | 225 |
| 13317 | CCGTTCCTCCAGCAACCGCC | 226 |
| 13318 | CGTTCCTCCAGCAACCGCCG | 227 |
| 13319 | GTTCCTCCAGCAACCGCCGC | 228 |
| 13320 | TTCCTCCAGCAACCGCCGCT | 229 |
| 13321 | TCCTCCAGCAACCGCCGCTA | 230 |
| 13322 | CCTCCAGCAACCGCCGCTAA | 231 |
| 13323 | CTCCAGCAACCGCCGCTAAG | 232 |
| 13324 | TCCAGCAACCGCCGCTAAGC | 233 |
| 13325 | CCAGCAACCGCCGCTAAGCC | 234 |
| 13326 | CAGCAACCGCCGCTAAGCCC | 235 |
| 13327 | AGCAACCGCCGCTAAGCCCG | 236 |
| 13328 | GCAACCGCCGCTAAGCCCGG | 237 |
| 13329 | CAACCGCCGCTAAGCCCGGC | 238 |
| 13330 | AACCGCCGCTAAGCCCGGCG | 239 |
| 13331 | ACCGCCGCTAAGCCCGGCGC | 240 |
| 13332 | CCGCCGCTAAGCCCGGCGCA | 241 |
| 13333 | CGCCGCTAAGCCCGGCGCAC | 242 |
| 13334 | GCCGCTAAGCCCGGCGCACC | 243 |
| 13335 | CCGCTAAGCCCGGCGCACCG | 244 |
| 13336 | CGCTAAGCCCGGCGCACCGC | 245 |
| 13337 | GCTAAGCCCGGCGCACCGCT | 246 |
| 13338 | CTAAGCCCGGCGCACCGCTC | 247 |
| 13339 | TAAGCCCGGCGCACCGCTCC | 248 |
| 13340 | AAGCCCGGCGCACCGCTCCA | 249 |
| 13341 | AGCCCGGCGCACCGCTCCAA | 250 |
| 13342 | GCCCGGCGCACCGCTCCAAC | 251 |
| 13343 | CCCGGCGCACCGCTCCAACC | 252 |
| 13344 | CCGGCGCACCGCTCCAACCT | 253 |
| 13345 | CGGCGCACCGCTCCAACCTG | 254 |
| 13346 | GGCGCACCGCTCCAACCTGT | 255 |
| 13347 | GCGCACCGCTCCAACCTGTT | 256 |
| 13348 | CGCACCGCTCCAACCTGTTC | 257 |
| 13349 | GCACCGCTCCAACCTGTTCT | 258 |
| 13350 | CACCGCTCCAACCTGTTCTC | 259 |
| 13351 | ACCGCTCCAACCTGTTCTCC | 260 |
| 13352 | CCGCTCCAACCTGTTCTCCA | 261 |
| 13353 | CGCTCCAACCTGTTCTCCAC | 262 |
| 13354 | ACGCCACCGCCGGCGCCAGG | 111 |
| 13355 | GACGCCACCGCCGGCGCCAG | 110 |
| 13356 | TGACGCCACCGCCGGCGCCA | 109 |
| 13357 | GTGACGCCACCGCCGGCGCC | 108 |
| 13358 | TGTGACGCCACCGCCGGCGC | 107 |
| 13359 | TTGTGACGCCACCGCCGGCG | 106 |
| 13360 | TTTGTGACGCCACCGCCGGC | 105 |
| 13361 | TTTTGTGACGCCACCGCCGG | 104 |
| 13362 | CTTTTGTGACGCCACCGCCG | 103 |
| 13363 | CCTTTTGTGACGCCACCGCC | 102 |
| 13364 | GCCTTTTGTGACGCCACCGC | 101 |
| 13365 | CGCCTTTTGTGACGCCACCG | 100 |
| 13366 | CCGCCTTTTGTGACGCCACC | 99 |
| 13367 | CCCGCCTTTTGTGACGCCAC | 98 |
| 13368 | TCCCGCCTTTTGTGACGCCA | 97 |
| 13369 | GTCCCGCCTTTTGTGACGCC | 96 |
| 13370 | GGTCCCGCCTTTTGTGACGC | 95 |
| 13371 | TGGTCCCGCCTTTTGTGACG | 94 |
| 13372 | GTGGTCCCGCCTTTTGTGAC | 93 |
| 13373 | TGTGGTCCCGCCTTTTGTGA | 92 |
| 13374 | CTGTGGTCCCGCCTTTTGTG | 91 |
| 13375 | ACTGTGGTCCCGCCTTTTGT | 90 |
| 13376 | CACTGTGGTCCCGCCTTTTG | 89 |
| 13377 | CCACTGTGGTCCCGCCTTTT | 88 |
| 13378 | ACCACTGTGGTCCCGCCTTT | 87 |
| 13379 | CACCACTGTGGTCCCGCCTT | 86 |
| 13380 | ACACCACTGTGGTCCCGCCT | 85 |
| 13381 | GACACCACTGTGGTCCCGCC | 84 |
| 13382 | GGACACCACTGTGGTCCCGC | 83 |
| 13383 | CGGACACCACTGTGGTCCCG | 82 |
| 13384 | TCGGACACCACTGTGGTCCC | 81 |
| 13385 | CTCGGACACCACTGTGGTCC | 80 |
| 13386 | TCTCGGACACCACTGTGGTC | 79 |
| 13387 | TTCTCGGACACCACTGTGGT | 78 |
| 13388 | CTTCTCGGACACCACTGTGG | 77 |
| 13389 | ACTTCTCGGACACCACTGTG | 76 |
| 13390 | GACTTCTCGGACACCACTGT | 75 |
| 13391 | TGACTTCTCGGACACCACTG | 74 |
| 13392 | CTGACTTCTCGGACACCACT | 73 |
| 13393 | CCTGACTTCTCGGACACCAC | 72 |
| 13394 | GCCTGACTTCTCGGACACCA | 71 |
| 13395 | TGCCTGACTTCTCGGACACC | 70 |
| 13396 | GTGCCTGACTTCTCGGACAC | 69 |
| 13397 | CGTGCCTGACTTCTCGGACA | 68 |
| 13398 | ACGTGCCTGACTTCTCGGAC | 67 |
| 13399 | TACGTGCCTGACTTCTCGGA | 66 |
| 13400 | CTACGTGCCTGACTTCTCGG | 65 |
| 13401 | GCTACGTGCCTGACTTCTCG | 64 |
| 13402 | AGCTACGTGCCTGACTTCTC | 63 |
| 13403 | GAGCTACGTGCCTGACTTCT | 62 |
| 13404 | TGAGCTACGTGCCTGACTTC | 61 |
| 13405 | CTGAGCTACGTGCCTGACTT | 60 |
| 13406 | GCTGAGCTACGTGCCTGACT | 59 |
| 13407 | CGCTGAGCTACGTGCCTGAC | 58 |
| 13408 | CCGCTGAGCTACGTGCCTGA | 57 |
| 13409 | GCCGCTGAGCTACGTGCCTG | 56 |
| 13410 | CGCCGCTGAGCTACGTGCCT | 55 |
| 13411 | CCGCCGCTGAGCTACGTGCC | 54 |
| 13412 | GCCGCCGCTGAGCTACGTGC | 53 |
| 13413 | GGCCGCCGCTGAGCTACGTG | 52 |
| 13414 | CGCGGCAGGTGAGAGGGGAGCTGCCC | 558 |

Hot Zones (Relative upstream location to gene start site)
1-1880
2150-2240
2420-3050
3230-4130
4310-4400

### Examples

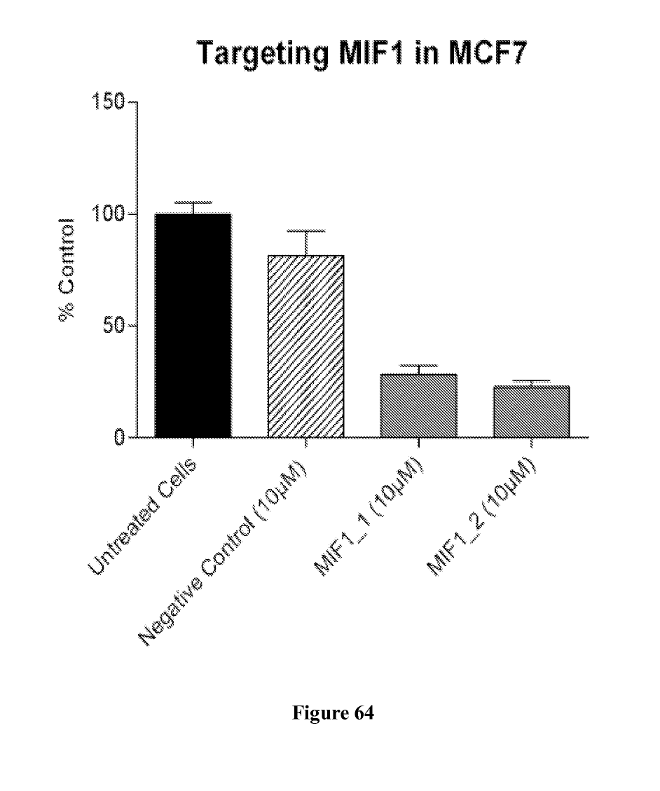

In FIG. 64, In MCF7 (human mammary breast cell line), MIF1_{—}1 (329) and MIF1_{—}2 (330) produced statistically significant (P<0.05) inhibition at 10 μM compared to the untreated and negative control values. The MIF1 sequences MIF1_{—}1 (329) and MIF1_{—}2 (330) fit the independent and dependent DNAi motif claims.

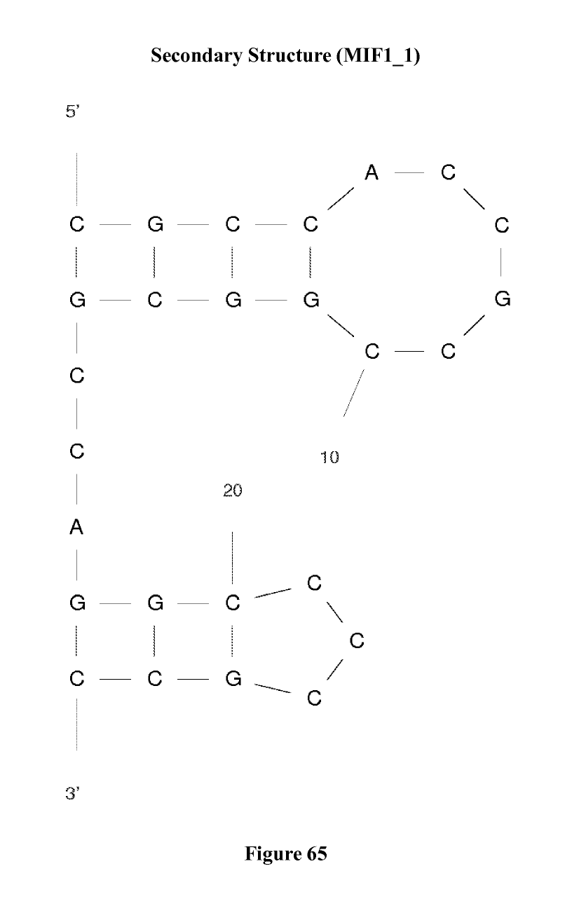

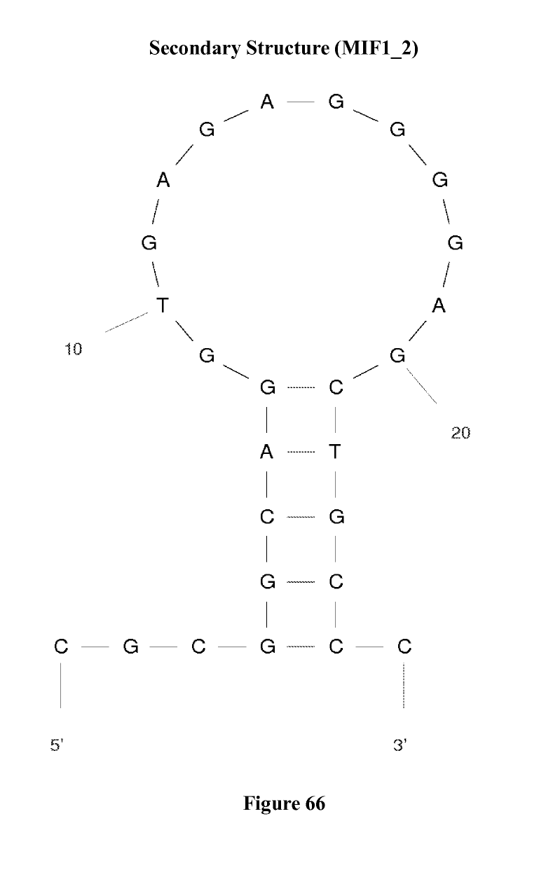

The secondary structure for MIF1_{—}1 (329) and MIF1_{—}2 (330) are shown in FIG. 65 and FIG. 66.

Genetic Code (5′ Upstream Region)

(SEQ ID NO: 13677)

CCATTCTGAGTATCTTCCAAGTGTTAGCTCCTTTAATCCTGGAAAGGACC
CCATGAAATTAGTACTTTTATTACCCCTGTTGTACATATGAGAGACTGAG
TAAAAGCCGGTGGCTTGTCCAGGGTCACACAGCTAACTGGAATGGCCAGG
AGTAGACCTGGTGACCATGGACCCCAGACCTTGATCACTGCACACGCTGC
GTCTGGGACCTCGCCTGGTACCTGAGGTCCGTGGCGCGCTGGTGCTGATC
ATTCAGAGTGCTCATGGGAAGTGTAGTCTAGAGTCTGTGTGCTTCCTGAT
CTCCTTGATCTCCATTTTATTGAGGAGGCCTTTAGGCCACCCGAGGGGTC
CAGAGTGACCCTGTGGATTAGCAGTGGAGCTCAGCTTGAGCCAGCGCTCT
TCAGGGGTCGTGTTCTGCCCCCATTCTCTGGTTCATTCTGCAGGTAGCAG
GGAATCATTGAAGATTAGAGAGAATCAAACACCTGGAGAGAGATGACTCT
GCCCGGGGAGCCCAGGCTCCTGTCTGGGTGCACACTCCAGGGCTAGATGG
TGACTTCTCAGCTACTCTAGCTTCATAGGCTCATAGTGCATGTGAGCACT
CATGTGGACACACGTGCACGCGCACACACATGGACACACACACACACACA
CACACCGCTGTCTTTGGAATCAGACCATGAAAATGCTTCCTCAGAGGCCT
AGGGGTGAGGAAGCTGAGGTGAGTTGTGCCTCCAGCTGGATGTGCTGGGA
TGGGGTGGGAGATGAGGTGGCCACACCTGGGTGGCAGGAACTCTGGGGCA
GTGAACCTTCTAACGAACAGATCTGGGATGCTGCCATGAGGAGGAAGAGG
GAGTCAGCAGCCATGCCTGCCAATGCCTCCTAGCGCATTTGTCCATGGTT
AGCGGATAATTATTGTGTCCCTATGGGTCCCAAGGTGTATTATTTTTTTT
TTGCTCTTATAATAAATCAACACAAATTTTTAGCAGCTTCAAACAACACG
CATTTATTATCTCACAGTTTCTGTGGGTCAGTAGTCCGGCGTGACATGAC
TAGGTCTTCTGTGTAAGGACTCGCATGGCCAAAGTCAAGGTATCTGAAGG
GACAAGGGAAAAATCCACTTCCAAGTTCAATCTGGTTGTGAGCAGAATTC
AGTTCCTTGTGGTTGTACCATGAGGTCTCTGGTCCCCTTCATCTTCAAAG
CCGGTAATGGACATCGAGTGTTTCTCTTGCTTGGAATCTGGCACTCTAGC
TGGAGAAAATTATCTGCTTTTAAGAGTTCATGTGATTAGATTGGGTGTAC
CCAGATGCTCCATGCTAATCTCCCTATTATGCACAGATGCATAATCCTAA
TTGCATCTGTGAAGTGCTTTTTGCCAGGTAACATGGCATACTTGTAGGTT
CCAGGGATTAGTGCTTGTCCTCCCCCTGCTATTCTTTAGTGGGCAGGGGG
TCATCTGCCTACCACGGAGGTAAGGGGTCAGGAGGTATGCATACAGCAAT
GCCCAAAAAGAGACTGTCCCCACTGGGATGGAGTTTACCGCCTAGACATG
CAGTCTTAACTCAGAAATATGGAGATAGCCTCGAAGGACAGGACAGGTAC
TGGGCACGTGTGGGAATGGACCAAGCCAGGTGCTCCGGGGGCTTTCCCAA
GGAACTAAGGCTGAGCCAAGAACTGAAGGATGAGTTGGAGTCAGATGAGG
GAAAATGTGGGCAAACTGGATTTCAGAACCAACCCCCAACCCTGGAGCCA
GGAGCCATGGTACTGAAGGACAGTGCGCCATAACTCAGAGAACCAGGGAG
GGTTGGCGGAGGCTCACAGGGACCGGGTTACCCCAGGGCCTTGTGACAGT
ACTACCCCTAGTATCAGAGGAGACTGTCATTGGCATTTAGGCCACTTGGT
GCTCATAACACCTCTATGTCAGGTGAACACTATTGTCATCCCCAAATTAC
AGATGGGGAAAGTGAGCCAAATGTCCATGCTAGTAAGAGGCAAATCATAT
CACTTCTTTGGGTACCCTTCTAGAAGGATGAGGCTGACTGCCACTGGAAA
CAGCTGGGGAGGGTACAAGGAGATGACAAGTGGCTCAGAGGCTGTCCTGG
CTATAAGAATTAAAGAGGAAAGAAACACCAAGGGTGGCTCGACAGTCAAC
AAGGACAGGTTTATTTTGGAAAACAAACTTGAGAGGGGCTTCTGGCCAAG
TTAGGTCAGAGCCACACTCTCTTACAAACTAAGGATATTTAAGGGTTTTG
GAGGGGGTTCTTATCATAGGTTCTGAATGTTTCTGTGTGAGGGAAAGTTT
ATTGCGGGGATGGAATGTCTCTGGTCAGAAGGGAGGCTGTCTCCGGGTTG
GCATGTTTCTGGTCAGAGAAGGGTTTATCTTAGGGTTGGAATGTTTCTGG
TTATGCTGACATTAGCTATTAGGCTGATATTTTCGGGCTGGATTTAGGCG
GCTTTTAATTAAGGGGGAACTTAGAATGGTGGTGTTTGTTCAAGATGGCA
ATGCTCCTGCTCCGTCACTGGCCAGGTAAGGCAACCCTTTGTTATGGTAA
CAACCTGAGATTGGCAGGGGCTCACCTCCAGGGGCAGCTCATGTGCTTGC
TGGCGAGGCTGCACCTTGTCATTCAGGTTCACAGGGCACAGGTCAACCAG
GCCCTGGCTCTTCAGTCTTCTGCCTGGAGTGACTTATGTAATTCTGCTCA
GCTTTCATAGGGCACAGGGAGTCGGGGCTAACTCTGCTGCCTGGGGCTGG
AAACAGACTCCTCCCTTGAGGAGCAGCAGTCCACCATAGGGAAGTCACAG
TGGTCCAGGCCAAAGGGGATGCAGGTAGTGTAGACTAGGCGGTAGTTCAG
GGAATGGAGAGAAGTGGGAATAAAGGGATAGTGAAAGGAAGCATATTTTA
CTGGCAGGTGATGAGGTGTAGGAGGACAAGTCATACATTTGGACTTTACA
GAGCAGTGGACACTCAGTCAGCTGCTGTCAGCGCCTGGGACTTAGGGGAG
TGCCCCTGGCTGGAGACATGGTATGGAGTGCCATCAGTTAGGGAGCCCTG
GGCACAGGTAAGAGAAGGTGTGACACCAGGAGGGAAAGAGTCTGGGGCCC
AGCTGCAGGAACCAATACCCATAGGCTATTTGTATAAATGGGCCATGGGG
CCTCCCAGCTGGAGGCTGGCTGGTGCCACGAGGGTCCCACAGGCATGGGT
GTCCTTCCTATATCACATGGCCTTCACTGAGACTGGTATATGGATTGCAC
CTATCAGAGACCAAGGACAGGACCTCCCTGGAAATCTCTGAGGACCTGGC
CTGTGATCCAGTTGCTGCCTTGTCCTCTTCCTGCTATGTCATGGCTTATC
TTCTTTCACCCATTCATTCATTCATTCATTCAGCAGTATTAGTCAATGTC
TCTTGTATGCCTGGCACCTGCTAGATGGTCCCCGAGTTTACCATTAGTGG
AAAAGACATTTAAGAAATTCACCAAGGGCTCTATGAGAGGCCATACACGG
TGGACCTGACTAGGGTGTGGCTTCCCTGAGGAGCTGAAGTTGCCCAGAGG
CCCAGAGAAGGGGAGCTGAGCACGTTTGAACCACTGAACCTGCTCTGGAC
CTCGCCTCCTTCCCTTCGGTGCCTCCCAGCATCCTATCCTCTTTAAAGAG
CAGGGGTTCAGGGAAGTTCCCTGGATGGTGATTCGCAGGGGCAGCTCCCC
TCTCACCTGCCGCGATGACTACCCCGCCCCATCTCAAACACACAAGCTCA
CGCATGCGGGACTGGAGCCCTTGAGGACATGTGGCCCAAAGACAGGAGGT
ACAGGGGCTCAGTGCGTGCAGTGGAATGAACTGGGCTTCATCTCTGGAAG
GGTAAGGGGCCATCTTCCGGGTTCACCGCCGCATCCCCACCCCCGGCACA
GCGCCTCCTGGCGACTAACATCGGTGACTTAGTGAAAGGACTAAGAAAGA
CCCGAGGCGAGGCCGGAACAGGCCGATTTCTAGCCGCCAAGTGGAGAACA
GGTTGGAGCGGTGCGCCGGGCTTAGCGGCGGTTGCTGGAGGAACGGGCGG
AGTCGCCCAGGGTCCTGCCCTGCGGGGGTCGAGCCGAGGCAGGCGGTGAC
TTCCCCACTCGGGGCGGAGCCGCAGCCTCGCGGGGGCGGGGCCTGGCGCC
GGCGGTGGCGTCACAAAAGGCGGGACCACAGTGGTGTCCGAGAAGTCAGG
CACGTAGCTCAGCGGCGGCCGCGGCGCGTGCGTCTGTGCCTCTGCGCGGG
TCTCCTGGTCCTTCTGCCATCATG

ERBB2

ERBB2 (also known as HER2/meu and CD340) is a receptor tyrosine kinase protein and member of the epidermal growth factor receptor family. ERBB2 contains extracellular, transmembrane, and intracellular domains. Ligand binding causes dimerization which activates downstream signaling pathways leading to proliferation, cell cycle progression, and cell survival promotion. ERBB2 is commonly associated with breast cancer where the gene is amplified or the protein is overexpressed leading to dysregulation of cell proliferation and survival. ERBB2 has also been associated with other cancers including lung and colorectal cancer.

Protein: ERBB2 (HER2) Gene: ERBB2 (Homo sapiens, chromosome 17, 37844167-37884915 [NCBI Reference Sequence: NC_{—}000017.10]; start site location: 37855813; strand: positive)

Gene Identification

| | |
|---|---|
| GeneID | 2064 |
| HGNC | 3430 |
| MIM | 164870 |

Targeted Sequences

| | | | Relative |
|---|---|---|---|
| | | | upstream |
| | | | location |
| | | | to gene |
| Sequence | Design | | start |
| ID No: | ID | Sequence (5′-3′) | site |
| 13415 | | CGGGAAGAGGATGCGCTGACCTGGC | 2571 |
| 13416 | | CACGCCCTGGGGAGGAGGCTCGAGAGG | 3267 |
| 13437 | | CGAGAGGGGCCGAGCCTCTGAAAAA | 3287 |
| 13452 | | CGTCTGGTCCACAGTCCGATGTCCA | 3944 |

Target Shift Sequences

| | | Relative |
|---|---|---|
| | | upstream |
| | | location to |
| Sequence | | gene |
| ID No: | Sequence (5′-3′) | start site |
| 13415 | CGGGAAGAGGATGCGCTGACCTGGC | 2571 |
| 13416 | CACGCCCTGGGGAGGAGGCTCGAGAGG | 3267 |
| 13417 | ACGCCCTGGGGAGGAGGCTC | 3268 |
| 13418 | CGCCCTGGGGAGGAGGCTCG | 3269 |
| 13419 | GCCCTGGGGAGGAGGCTCGA | 3270 |
| 13420 | CCCTGGGGAGGAGGCTCGAG | 3271 |
| 13421 | TCACGCCCTGGGGAGGAGGC | 3266 |
| 13422 | CTCACGCCCTGGGGAGGAGG | 3265 |
| 13423 | ACTCACGCCCTGGGGAGGAG | 3264 |
| 13424 | AACTCACGCCCTGGGGAGGA | 3263 |
| 13425 | GAACTCACGCCCTGGGGAGG | 3262 |
| 13426 | AGAACTCACGCCCTGGGGAG | 3261 |
| 13427 | CAGAACTCACGCCCTGGGGA | 3260 |
| 13428 | TCAGAACTCACGCCCTGGGG | 3259 |
| 13429 | GTCAGAACTCACGCCCTGGG | 3258 |
| 13430 | GGTCAGAACTCACGCCCTGG | 3257 |
| 13431 | GGGTCAGAACTCACGCCCTG | 3256 |
| 13432 | GGGGTCAGAACTCACGCCCT | 3255 |
| 13433 | TGGGGTCAGAACTCACGCCC | 3254 |
| 13434 | CTGGGGTCAGAACTCACGCC | 3253 |
| 13435 | GCTGGGGTCAGAACTCACGC | 3252 |
| 13436 | AGCTGGGGTCAGAACTCACG | 3251 |
| 13437 | CGAGAGGGGCCGAGCCTCTGAAAAA | 3287 |
| 13438 | GAGAGGGGCCGAGCCTCTGA | 3288 |
| 13439 | AGAGGGGCCGAGCCTCTGAA | 3289 |
| 13440 | GAGGGGCCGAGCCTCTGAAA | 3290 |
| 13441 | AGGGGCCGAGCCTCTGAAAA | 3291 |
| 13442 | GGGGCCGAGCCTCTGAAAAA | 3292 |
| 13443 | GGGCCGAGCCTCTGAAAAAG | 3293 |
| 13444 | GGCCGAGCCTCTGAAAAAGA | 3294 |
| 13445 | GCCGAGCCTCTGAAAAAGAA | 3295 |
| 13446 | CCGAGCCTCTGAAAAAGAAT | 3296 |
| 13447 | CGAGCCTCTGAAAAAGAATG | 3297 |
| 13448 | TCGAGAGGGGCCGAGCCTCT | 3286 |
| 13449 | CTCGAGAGGGGCCGAGCCTC | 3285 |
| 13450 | GCTCGAGAGGGGCCGAGCCT | 3284 |
| 13451 | GGCTCGAGAGGGGCCGAGCC | 3283 |
| 13452 | CGTCTGGTCCACAGTCCGATGTCCA | 3944 |
| 13453 | GTCTGGTCCACAGTCCGATG | 3945 |
| 13454 | TCTGGTCCACAGTCCGATGT | 3946 |
| 13455 | CTGGTCCACAGTCCGATGTC | 3947 |
| 13456 | TGGTCCACAGTCCGATGTCC | 3948 |
| 13457 | GGTCCACAGTCCGATGTCCA | 3949 |
| 13458 | GTCCACAGTCCGATGTCCAG | 3950 |
| 13459 | TCCACAGTCCGATGTCCAGG | 3951 |
| 13460 | CCACAGTCCGATGTCCAGGC | 3952 |
| 13461 | CACAGTCCGATGTCCAGGCC | 3953 |
| 13462 | ACAGTCCGATGTCCAGGCCA | 3954 |
| 13463 | CAGTCCGATGTCCAGGCCAC | 3955 |
| 13464 | AGTCCGATGTCCAGGCCACA | 3956 |
| 13465 | GTCCGATGTCCAGGCCACAA | 3957 |
| 13466 | TCCGATGTCCAGGCCACAAA | 3958 |
| 13467 | CCGATGTCCAGGCCACAAAC | 3959 |
| 13468 | CGATGTCCAGGCCACAAACT | 3960 |
| 13469 | TCGTCTGGTCCACAGTCCGA | 3943 |
| 13470 | GTCGTCTGGTCCACAGTCCG | 3942 |
| 13471 | AGTCGTCTGGTCCACAGTCC | 3941 |
| 13472 | GAGTCGTCTGGTCCACAGTC | 3940 |
| 13473 | GGAGTCGTCTGGTCCACAGT | 3939 |
| 13474 | AGGAGTCGTCTGGTCCACAG | 3938 |
| 13475 | GAGGAGTCGTCTGGTCCACA | 3937 |
| 13476 | GGAGGAGTCGTCTGGTCCAC | 3936 |
| 13477 | GGGAGGAGTCGTCTGGTCCA | 3935 |
| 13478 | CGGGAGGAGTCGTCTGGTCC | 3934 |
| 13479 | TCGGGAGGAGTCGTCTGGTC | 3933 |
| 13480 | ATCGGGAGGAGTCGTCTGGT | 3932 |
| 13481 | AATCGGGAGGAGTCGTCTGG | 3931 |
| 13482 | AAATCGGGAGGAGTCGTCTG | 3930 |
| 13483 | GAAATCGGGAGGAGTCGTCT | 3929 |

Hot Zones (Relative upstream location to gene start site)
100-4510

### Examples

Genetic Code (5′ Upstream Region)

(SEQ ID NO: 13678)

GGGGGCACCAGTAGAATGGCCAGGACAAACGCAGTGCAGCACAGAGACTC
AGACCCTGGCAGCCATGCCTGCGCAGGCAGTGATGAGAGTGACATGTACT
GTTGTGGACATGCACAAAAGTGAGGTGAGTCGCAGGACAGAAGAGTGCTT
TTTGTTTCAGCAGAGCAGCCTGGGGAGAGATAAAAGCTACTCCTGGGGCC
TGGGCCTGCATTCCTGAGATGTGGGTAAGAGGGGCCCAGGGTCAGAGTGT
CTGGCAAGCTTGGCTCTGCCCCTTTGCTGTCCTGGAGACTAGGGCTAATC
CTGGGCTCAGGGAGTGGCCTCCCCATGGTTAGGATACAAGTGCTCATCAA
GGGCCACCCCTAGGAAGGACCAATTTTCCTATCAGAAGCTTCTAAGTTAT
CCTCCTTTGGCCCAAAGGGACACCTCAAGCCTACTCTGAGGAACTCTTTC
CAATGAACTAATTCCTACAGTCACTTCCCCAGCAACCTGTGCCTCAGCCT
CAAGGCACTGTGGGGTAGGCCTCAGTTTGTGGCCTGGACATCGGACTGTG
GACCAGACGACTCCTCCCGATTTCTGTTTGTTTTCAGTCCTCTGACCCCA
AGCTGGCTGGTGAAGTAGGTAGAGGGAGGAGACTTTGGTGCATGCATACA
CACACACACACACACACACACACACACACACACACACACACACACACACA
CGTCTCCTGTGCCCCCCAGTCTCCATGGCTGGTCAATGATTGACTGGCAT
TTCACAGGCCGCTGGTTGCAGCCCCAGCCTGTTGACTTAGAGGTCACCCT
CGGAAGCTAGAGCCCTGTCCTGCCTCTTCAGTGTCAGTGGTCACTCCACT
GCCCACAGGCTGGGGTCTTGGGCAAAACACACGCATCTGCCCTGATCTGA
GTTTGCTGCCCTCTGTCCCGCAGTCAGCCCCACTCTGTTCCCACTCCCTC
TCCCCAGCCCCCTAGCTAGACCCCTCTCACCAGCACCCCTTTCCCTTCCC
TGAGGGTCCCCCTCGCTGTCTTTGTCCCTCAGACATCCTCTTTCCTGGGC
TCTCCTGCCAGGCCCTGCTGGAGGGACAGTTAAGGAGGAAATCGAATCAG
CAGCGCCCACCCCTGCCCCCCTTCCTCTCCTCTTGTCAGACACCAGACGA
GGTTTTTTCCTCTGGCTTCCCAGCTCTGAATGGGCTCATTCTTTTTCAGA
GGCTCGGCCCCTCTCGAGCCTCCTCCCCAGGGCGTGAGTTCTGACCCCAG
CTCCTCCCCCCATCCCCACTCCAGCCCCCTCTCCAGCTTGCTCCACCCTC
TCTACCGCCCACCGGGACTGGGCATTGTCTGCCAGTCCGGGTTTCTTCCT
GGGATTTGGGATGCAGAGAGGATGGGTTTGCTTGGGCGGGGGGGTGGAGA
GTGAAGGGGGGAAGCAGGATCTTTGTAGAGGGAGGGACCTACAGTTACCT
GGACTTCTTTCCTCTGTCTCCCCTCTTGGTACCCTTGACTGGGGCTCTTG
AGGGTAATGGGTGAAGCCAAATCTGCCATGGCTCAGTTCCCAGCTCAGCT
CTGTGACCTTGGGAAAGTTCCTTTAGCTCGTGGAATCTCAAGGCTCAAGG
TTCCTCTTCTGCAAAATGGGGAATGATAACACCTGCCTCCTCTGGAGTCT
TGGGGACTCAGTGTTCTGAGGAACGTGGCTGTAGGTCAGAGTGGCACAGA
GTAGGGTCCAATGAAGCATGGCGTCCACAGTAGCTTTCCTGACTGGACTA
ACCTTTCCGGACACAACAGCAGGGCAGGGGTGGGGCCTGGGGAGAAAGGA
CACCTCTAACCCTGATCCTAACATCCCGATGGCCTCTAAGGCTGCCTGCA
CACTCATCCAGGTGCAAGCCCTCCAAGGTGTGGTGTGATGAACCAGTGAC
TCCTGGAGCCAGGTCAGCGCATCCTCTTCCCGCAGGGCTGTAAGCTGCAG
GACTGAGAGGCAGGTTGACCAGGTCCTGGGCTGGATGATGGGGTGAGAGT
AAGGGGTCAGTTTTGATACATGCCCAACTTTTCTCTCTAGCCCTAAGACA
TCCTGGGCAAATTGCTTACCTCAGTTCCCCTGATCCTCACCCTAACCCTA
ACACCAGCTCAAGAGAAAATAGGGATATTGATGGCCATCCAGAAGGGCTG
CTGTGTTCCATACACAGCAATATTTCTCGAATGTTTGTGACAGCGGTCCA
AGGAATAAGTTAATTTTACATTATCACTCTGGATACCTGTACAAAACTCC
ACCTTATCCTTACTATATGAATGTGCTAGGGTTGTTTTTTTGTTTTGTTT
TTTTTTTTTTTTTTTGAGACAGAGTTTCGCTCTTGTTGCCCAGGCTGGAG
TACAATGGCGCGATCTTGGCTCACCGCAACCTCCGCTTCCCAGGTTCAAG
CGATTCACCTGCCTCAGCCTTCCCGAGTAGCTGGGATTACAGGCATGCGC
CACCATGCCCGGCTAATTTTGTGTTTTTAGTAGAGACAGGGTTTCTCCAT
GTTGGTCAGGCTGGTACCAAACTCCCGACCTCAGGTGATCCACCTGCCTT
GGCCTCCCAAAGTGCTGCAATTACAGGCATGAGCCACCGCACCCAGCCGT
GCTAGGGTCTTTTTCTGTTCAATTCCTTTCTCTCTCTTGCTCTCTTTCTT
TCTTTCAATGGAGTCTTACTCTGTCACCCAGGCTGGAGTGCAGTGGCAAG
ATCTCAGCTCACTGCAACCTCTGCCCTCTGAGTTCAAGCAATTCTCCTGC
CTCAGCCTCCCGAGTAGCTGGGATTACAGGTGCCTGCCACCACACCTAGT
TAATTTTTGTACTTTTAGTAGAGATGGGGTTTTGTCATGTTGGCCAGGCT
GGTCTCGAACTCCTGACCTCGTGATCTGCCTGTCTTGGCCTCCCAAAGTG
CTGGGATTACAGGCATGAGCCGCCATACTCGGCCAACTTTGTATTACTTT
CTTAAAGAGAGTTTCCCAAATTATATAAGCTTCAGGCCCCACAAAACCTA
GATCTGCCCCAGTATAACTAAATCTGGGACCATTTATTGAGCAATTATTA
TGTGCCAAGTATTGCGCTGAGTGCTTCCAGAGCATTATCTCCTTTAACCC
CAGCATAGTATGTCAGATGCTGTTTTACAGATGAGCCAACTGAGACCAGA
GATGCTCAGTCACTTGCCCAAGGTGACATGACTGATATGGAATAGAGTCA
AGATTTTTTTTTTTTTTTTTGACACGGAGTCTCACTCTGTCTCCCAGGCT
GGAGTGCAGAGGCGCAATCTCAGCTCACTGCAAGCTCTGCCTCCCAGGTT
CACGCCATTCTCCTGCCTCAGCCTCCTGAGTAGCTGGGACTACAGGCACC
CGCCACCACACCTGGCTAATTTTTTGTATTTTTAGCAGAGACAGGGTTTC
ACCGTGTTAGCCAGGATGGTCTCGATCTCCTGACCTCGTGATCTGCCTGC
CTCGGCCTCCCAAAGTGCTGGAATTACAGGTGTGAGCCACCGCGACTGGC
CAGATTCAAGATTTGAACCCAGGTCCTCTTGGTCCCAGAGGCCCCTGTTT
CTCAACTCCCTAGGATGGCATAGCAACCTGTCCCACAAGAGGTGCCTGCT
TTAAGTGTGCTCAGCACATGGAAGCAAGTTTAGAAATGCAAGTGTATACC
TGTAAAGAGGTGTGGGAGATGGGGGGGAGGGAAGAGAGAAAGAGATGCTG
GTGTCCTTCATTCTCCAGTCCCTGATAGGTGCCTTTGATCCCTTCTTGAC
CAGTATAGCTGCATTCTTGGCTGGGGCATTCCAACTAGAACTGCCAAATT
TAGCACATAAAAATAAGGAGGCCCAGTTAAATTTGAATTTCAGATAAACA
ATGAATAATTTGTTAGTATAAATATGTCCCATGCAATATCTTGTTGAAAT
TAAAAAAAAAAAAAAAAGTCTTCCTTCCATCCCCACCCCTACCACTAGGC
CTAAGGAATAGGGTCAGGGGCTCCAAATAGAATGTGGTTGAGAAGTGGAA
TTAAGCAGGCTAATAGAAGGCAAGGGGCAAAGAAGAAACCTTGAATGCAT
TGGGTGCTGGGTGCCTCCTTAAATAAGCAAGAAGGGTGCATTTTGAAGAA
TTGAGATAGAAGTCTTTTTGGGCTGGGTGCAGTTGCTCGTGGTTGTAATT
CCAGCACTTTGGGAGGCTGAGGCGGGAGGATCACCTGAGGTTGGGAGTTC
AAGACCAGCCTCACCAACGTGGAGAAACCCTGTCTTTACTAAAAATACAA
AAAATTAGCTGGTCATGGTGGCACATGCCTGTAATCCCAGCTGCTCGGGA
GGCTGAGGCAGGAGAATCACTTGAACCAGGGAGGCAGAGGTTGTGGTGAG
CAGAGATCGCGCCATTGCTCTCCAGCCTGGGCAACAAGAGCAAAAGTTCG
TTTAAAAAAAAAAAAAAGTCCTTTCGATGTGACTGTCTCCTCCCAAATTT
GTAGACCCTCTTAAGATCATGCTTTTCAGATACTTCAAAGATTCCAGAAG
ATATG

FGFR1

FGFR1 (fibroblast growth factor receptor 1) is a 100-135 kDa glycoprotein receptor tyrosine kinase specific for the fibroblast growth factor family. The FGFR1 receptor has an extracellular, transmembrane, and intracellular domain. The extracellular domain includes a single peptide and two or three Ig-like domains. The intracellular domain includes two tyrosine kinase subdomains. Stimulation of the FGFR1 receptor eventually has an effect on mitogenesis and differentiation. Specifically, FGFR1 has been associated with various diseases including Pfeiffer syndrome, various cancers, Kallmann syndrome, and osteoglyphic dysplasia.

Protein: FGFR1 Gene: FGFR1 (Homo sapiens, chromosome 8, 38411138-38468834 [NCBI Reference Sequence: NC_{—}000008.11]; start site location: 38314964; strand: negative)

Gene Identification

| | |
|---|---|
| GeneID | 2260 |
| HGNC | 3688 |
| HPRD | 00634 |
| MIM | 136350 |

Targeted Sequences

| | | | Relative |
|---|---|---|---|
| | | | upstream |
| | | | location |
| Se- | | | to gene |
| quence | Design | | start |
| ID No: | ID | Sequence (5′-3′) | site |
| 13484 | | CGAGCCAGGCAGGGCCCCTCGCAAGTG | 1850 |
| 13522 | | GACGGATATGAGTCCAGAAGTTGCG | 1472 |
| 13535 | | TAGCTGCGTGCAGTGGCGCGCGCCTGT | 4910 |
| 13561 | | CCGCCTCGCCAGCTCCCGAGCGCGAGTT | 10239 |
| 13655 | | CGCCTCCTCCCAGGTGTGGGCTGGCTGCA | 3067 |
| | | GACCG | |

Target Shift Sequences

| | | Relative |
|---|---|---|
| | | upstream |
| | | location |
| Sequence | | to gene |
| ID | | start |
| No: | Sequence (5′-3′) | site |
| 13484 | CGAGCCAGGCAGGGCCCCTCGCAAGTG | 1850 |
| 13485 | GAGCCAGGCAGGGCCCCTCG | 1851 |
| 13486 | AGCCAGGCAGGGCCCCTCGC | 1852 |
| 13487 | GCCAGGCAGGGCCCCTCGCA | 1853 |
| 13488 | CCAGGCAGGGCCCCTCGCAA | 1854 |
| 13489 | CAGGCAGGGCCCCTCGCAAG | 1855 |
| 13490 | AGGCAGGGCCCCTCGCAAGT | 1856 |
| 13491 | GGCAGGGCCCCTCGCAAGTG | 1857 |
| 13492 | GCAGGGCCCCTCGCAAGTGA | 1858 |
| 13493 | CAGGGCCCCTCGCAAGTGAG | 1859 |
| 13494 | AGGGCCCCTCGCAAGTGAGT | 1860 |
| 13495 | GGGCCCCTCGCAAGTGAGTC | 1861 |
| 13496 | GGCCCCTCGCAAGTGAGTCA | 1862 |
| 13497 | GCCCCTCGCAAGTGAGTCAG | 1863 |
| 13498 | CCCCTCGCAAGTGAGTCAGT | 1864 |
| 13499 | CCCTCGCAAGTGAGTCAGTG | 1865 |
| 13500 | CCTCGCAAGTGAGTCAGTGC | 1866 |
| 13501 | CTCGCAAGTGAGTCAGTGCT | 1867 |
| 13502 | TCGCAAGTGAGTCAGTGCTG | 1868 |
| 13503 | CGCAAGTGAGTCAGTGCTGG | 1869 |
| 13504 | CCGAGCCAGGCAGGGCCCCT | 1849 |
| 13505 | CCCGAGCCAGGCAGGGCCCC | 1848 |
| 13506 | CCCCGAGCCAGGCAGGGCCC | 1847 |
| 13507 | CCCCCGAGCCAGGCAGGGCC | 1846 |
| 13508 | TCCCCCGAGCCAGGCAGGGC | 1845 |
| 13509 | CTCCCCCGAGCCAGGCAGGG | 1844 |
| 13510 | CCTCCCCCGAGCCAGGCAGG | 1843 |
| 13511 | GCCTCCCCCGAGCCAGGCAG | 1842 |
| 13512 | TGCCTCCCCCGAGCCAGGCA | 1841 |
| 13513 | CTGCCTCCCCCGAGCCAGGC | 1840 |
| 13514 | CCTGCCTCCCCCGAGCCAGG | 1839 |
| 13515 | CCCTGCCTCCCCCGAGCCAG | 1838 |
| 13516 | GCCCTGCCTCCCCCGAGCCA | 1837 |
| 13517 | AGCCCTGCCTCCCCCGAGCC | 1836 |
| 13518 | CAGCCCTGCCTCCCCCGAGC | 1835 |
| 13519 | TCAGCCCTGCCTCCCCCGAG | 1834 |
| 13520 | TTCAGCCCTGCCTCCCCCGA | 1833 |
| 13521 | CTTCAGCCCTGCCTCCCCCG | 1832 |
| 13522 | GACGGATATGAGTCCAGAAGTTGCG | 1472 |
| 13523 | ACGGATATGAGTCCAGAAGT | 1473 |
| 13524 | CGGATATGAGTCCAGAAGTT | 1474 |
| 13525 | TGACGGATATGAGTCCAGAA | 1471 |
| 13526 | CTGACGGATATGAGTCCAGA | 1470 |
| 13527 | TCTGACGGATATGAGTCCAG | 1469 |
| 13528 | GTCTGACGGATATGAGTCCA | 1468 |
| 13529 | TGTCTGACGGATATGAGTCC | 1467 |
| 13530 | ATGTCTGACGGATATGAGTC | 1466 |
| 13531 | GATGTCTGACGGATATGAGT | 1465 |
| 13532 | TGATGTCTGACGGATATGAG | 1464 |
| 13533 | GTGATGTCTGACGGATATGA | 1463 |
| 13534 | AGTGATGTCTGACGGATATG | 1462 |
| 13535 | TAGCTGCGTGCAGTGGCGCGCGCCTGT | 4910 |
| 13536 | AGCTGCGTGCAGTGGCGCGC | 4911 |
| 13537 | GCTGCGTGCAGTGGCGCGCG | 4912 |
| 13538 | CTGCGTGCAGTGGCGCGCGC | 4913 |
| 13539 | TGCGTGCAGTGGCGCGCGCC | 4914 |
| 13540 | GCGTGCAGTGGCGCGCGCCT | 4915 |
| 13541 | CGTGCAGTGGCGCGCGCCTG | 4916 |
| 13542 | GTGCAGTGGCGCGCGCCTGT | 4917 |
| 13543 | TGCAGTGGCGCGCGCCTGTA | 4918 |
| 13544 | GCAGTGGCGCGCGCCTGTAG | 4919 |
| 13545 | CAGTGGCGCGCGCCTGTAGT | 4920 |
| 13546 | AGTGGCGCGCGCCTGTAGTC | 4921 |
| 13547 | GTGGCGCGCGCCTGTAGTCC | 4922 |
| 13548 | TGGCGCGCGCCTGTAGTCCC | 4923 |
| 13549 | GGCGCGCGCCTGTAGTCCCA | 4924 |
| 13550 | GCGCGCGCCTGTAGTCCCAG | 4925 |
| 13551 | CGCGCGCCTGTAGTCCCAGC | 4926 |
| 13552 | GCGCGCCTGTAGTCCCAGCT | 4927 |
| 13553 | CGCGCCTGTAGTCCCAGCTA | 4928 |
| 13554 | GCGCCTGTAGTCCCAGCTAC | 4929 |
| 13555 | CGCCTGTAGTCCCAGCTACT | 4930 |
| 13556 | TTAGCTGCGTGCAGTGGCGC | 4909 |
| 13557 | ATTAGCTGCGTGCAGTGGCG | 4908 |
| 13558 | AATTAGCTGCGTGCAGTGGC | 4907 |
| 13559 | AAATTAGCTGCGTGCAGTGG | 4906 |
| 13560 | AAAATTAGCTGCGTGCAGTG | 4905 |
| 13561 | CCGCCTCGCCAGCTCCCGAGCGCGAGTT | 10239 |
| 13562 | CGCCTCGCCAGCTCCCGAGC | 10240 |
| 13563 | GCCTCGCCAGCTCCCGAGCG | 10241 |
| 13564 | CCTCGCCAGCTCCCGAGCGC | 10242 |
| 13565 | CTCGCCAGCTCCCGAGCGCG | 10243 |
| 13566 | TCGCCAGCTCCCGAGCGCGA | 10244 |
| 13567 | CGCCAGCTCCCGAGCGCGAG | 10245 |
| 13568 | GCCAGCTCCCGAGCGCGAGT | 10246 |
| 13569 | CCAGCTCCCGAGCGCGAGTT | 10247 |
| 13570 | CAGCTCCCGAGCGCGAGTTG | 10248 |
| 13571 | AGCTCCCGAGCGCGAGTTGG | 10249 |
| 13572 | GCTCCCGAGCGCGAGTTGGA | 10250 |
| 13573 | CTCCCGAGCGCGAGTTGGAG | 10251 |
| 13574 | TCCCGAGCGCGAGTTGGAGG | 10252 |
| 13575 | CCCGAGCGCGAGTTGGAGGA | 10253 |
| 13576 | GCCGCCTCGCCAGCTCCCGA | 10238 |
| 13577 | CGCCGCCTCGCCAGCTCCCG | 10237 |
| 13578 | CCGCCGCCTCGCCAGCTCCC | 10236 |
| 13579 | GCCGCCGCCTCGCCAGCTCC | 10235 |
| 13580 | CGCCGCCGCCTCGCCAGCTC | 10234 |
| 13581 | CCGCCGCCGCCTCGCCAGCT | 10233 |
| 13582 | GCCGCCGCCGCCTCGCCAGC | 10232 |
| 13583 | AGCCGCCGCCGCCTCGCCAG | 10231 |
| 13584 | GAGCCGCCGCCGCCTCGCCA | 10230 |
| 13585 | GGAGCCGCCGCCGCCTCGCC | 10229 |
| 13586 | AGGAGCCGCCGCCGCCTCGC | 10228 |
| 13587 | GAGGAGCCGCCGCCGCCTCG | 10227 |
| 13588 | TGAGGAGCCGCCGCCGCCTC | 10226 |
| 13589 | CTGAGGAGCCGCCGCCGCCT | 10225 |
| 13590 | ACTGAGGAGCCGCCGCCGCC | 10224 |
| 13591 | CACTGAGGAGCCGCCGCCGC | 10223 |
| 13592 | TCACTGAGGAGCCGCCGCCG | 10222 |
| 13593 | CTCACTGAGGAGCCGCCGCC | 10221 |
| 13594 | ACTCACTGAGGAGCCGCCGC | 10220 |
| 13595 | GACTCACTGAGGAGCCGCCG | 10219 |
| 13596 | GGACTCACTGAGGAGCCGCC | 10218 |
| 13597 | GGGACTCACTGAGGAGCCGC | 10217 |
| 13598 | CGGGACTCACTGAGGAGCCG | 10216 |
| 13599 | CCGGGACTCACTGAGGAGCC | 10215 |
| 13600 | CCCGGGACTCACTGAGGAGC | 10214 |
| 13601 | TCCCGGGACTCACTGAGGAG | 10213 |
| 13602 | CTCCCGGGACTCACTGAGGA | 10212 |
| 13603 | CCTCCCGGGACTCACTGAGG | 10211 |
| 13604 | CCCTCCCGGGACTCACTGAG | 10210 |
| 13605 | TCCCTCCCGGGACTCACTGA | 10209 |
| 13606 | GTCCCTCCCGGGACTCACTG | 10208 |
| 13607 | TGTCCCTCCCGGGACTCACT | 10207 |
| 13608 | CTGTCCCTCCCGGGACTCAC | 10206 |
| 13609 | CCTGTCCCTCCCGGGACTCA | 10205 |
| 13610 | GCCTGTCCCTCCCGGGACTC | 10204 |
| 13611 | GGCCTGTCCCTCCCGGGACT | 10203 |
| 13612 | GGGCCTGTCCCTCCCGGGAC | 10202 |
| 13613 | CGGGCCTGTCCCTCCCGGGA | 10201 |
| 13614 | CCGGGCCTGTCCCTCCCGGG | 10200 |
| 13615 | CCCGGGCCTGTCCCTCCCGG | 10199 |
| 13616 | CCCCGGGCCTGTCCCTCCCG | 10198 |
| 13617 | GCCCCGGGCCTGTCCCTCCC | 10197 |
| 13618 | CGCCCCGGGCCTGTCCCTCC | 10196 |
| 13619 | TCGCCCCGGGCCTGTCCCTC | 10195 |
| 13620 | TTCGCCCCGGGCCTGTCCCT | 10194 |
| 13621 | CTTCGCCCCGGGCCTGTCCC | 10193 |
| 13622 | CCTTCGCCCCGGGCCTGTCC | 10192 |
| 13623 | GCCTTCGCCCCGGGCCTGTC | 10191 |
| 13624 | CGCCTTCGCCCCGGGCCTGT | 10190 |
| 13625 | CCGCCTTCGCCCCGGGCCTG | 10189 |
| 13626 | GCCGCCTTCGCCCCGGGCCT | 10188 |
| 13627 | CGCCGCCTTCGCCCCGGGCC | 10187 |
| 13628 | TCGCCGCCTTCGCCCCGGGC | 10186 |
| 13629 | CTCGCCGCCTTCGCCCCGGG | 10185 |
| 13630 | CCTCGCCGCCTTCGCCCCGG | 10184 |
| 13631 | GCCTCGCCGCCTTCGCCCCG | 10183 |
| 13632 | GGCCTCGCCGCCTTCGCCCC | 10182 |
| 13633 | GGGCCTCGCCGCCTTCGCCC | 10181 |
| 13634 | CGGGCCTCGCCGCCTTCGCC | 10180 |
| 13635 | GCGGGCCTCGCCGCCTTCGC | 10179 |
| 13636 | CGCGGGCCTCGCCGCCTTCG | 10178 |
| 13637 | CCGCGGGCCTCGCCGCCTTC | 10177 |
| 13638 | ACCGCGGGCCTCGCCGCCTT | 10176 |
| 13639 | AACCGCGGGCCTCGCCGCCT | 10175 |
| 13640 | AAACCGCGGGCCTCGCCGCC | 10174 |
| 13641 | GAAACCGCGGGCCTCGCCGC | 10173 |
| 13642 | GGAAACCGCGGGCCTCGCCG | 10172 |
| 13643 | AGGAAACCGCGGGCCTCGCC | 10171 |
| 13644 | CAGGAAACCGCGGGCCTCGC | 10170 |
| 13645 | CCAGGAAACCGCGGGCCTCG | 10169 |
| 13646 | TCCAGGAAACCGCGGGCCTC | 10168 |
| 13647 | GTCCAGGAAACCGCGGGCCT | 10167 |
| 13648 | AGTCCAGGAAACCGCGGGCC | 10166 |
| 13649 | CAGTCCAGGAAACCGCGGGC | 10165 |
| 13650 | CCAGTCCAGGAAACCGCGGG | 10164 |
| 13651 | CCCAGTCCAGGAAACCGCGG | 10163 |
| 13652 | CCCCAGTCCAGGAAACCGCG | 10162 |
| 13653 | TCCCCAGTCCAGGAAACCGC | 10161 |
| 13654 | CTCCCCAGTCCAGGAAACCG | 10160 |
| 13655 | CGCCTCCTCCCAGGTGTGGGCTGGCTGCAGACCG | 3067 |
| 13656 | CCGCCTCCTCCCAGGTGTGG | 3066 |
| 13657 | GCCGCCTCCTCCCAGGTGTG | 3065 |
| 13658 | TGCCGCCTCCTCCCAGGTGT | 3064 |
| 13659 | CTGCCGCCTCCTCCCAGGTG | 3063 |
| 13660 | CCTGCCGCCTCCTCCCAGGT | 3062 |
| 13661 | GCCTGCCGCCTCCTCCCAGG | 3061 |
| 13662 | AGCCTGCCGCCTCCTCCCAG | 3060 |
| 13663 | AAGCCTGCCGCCTCCTCCCA | 3059 |
| 13664 | AAAGCCTGCCGCCTCCTCCC | 3058 |
| 13665 | AAAAGCCTGCCGCCTCCTCC | 3057 |
| 13666 | GAAAAGCCTGCCGCCTCCTC | 3056 |
| 13667 | AGAAAAGCCTGCCGCCTCCT | 3055 |
| 13668 | CAGAAAAGCCTGCCGCCTCC | 3054 |
| 13669 | CCAGAAAAGCCTGCCGCCTC | 3053 |
| 13670 | CCCAGAAAAGCCTGCCGCCT | 3052 |
| 13671 | CCCCAGAAAAGCCTGCCGCC | 3051 |
| 13672 | TCCCCAGAAAAGCCTGCCGC | 3050 |
| 13673 | GTCCCCAGAAAAGCCTGCCG | 3049 |

Hot Zones (Relative upstream location to gene start site)
1350-1500
1750-1900
2500-5500
10150-10300

### Examples

Genetic Code (5′ Upstream Region)

(SEQ ID NO: 13679)

AGCTGGCAGGGCGAAGGGCCGACAAATCCTCCCTGACCCTCCCAGCTCTT
TGTTATCTCAGAGGGAAGGTTACATTTCTGTATGGGAGGCAAGGTGCCAG
GAGGCCTCGGGCAGAACAGAGACAGGCAGAGCTGCTGTCTGACCCCTGTT
GCCTGGAGCAGCTCAGGGCTGCCCTAGGGACACTCTCCCTCCACTGGCCT
GGGGCCCTTCCAGAAATGGGAGGGCTACATTTCAGAAAGAGGGCGAGTAG
AGGAGTGGGACAGAAAAGGAGCGAGGTGGGCTGGAAGGATAAAAGCAGCC
AACTCTCAATTATTCAGAAACCTGTCTGCAGTGTGTGGACAGCCCATGCC
TTTGCTGAGTTTCTCACCTTCTCTGTTCAGCTGCCATCAGCTCTTTCCCT
GAGAAGTGGAGGAGGGACCCTGGCAAGTTGGCCACTTGCTTTCATTTTGG
CTTCTTGATAAATCTATAGAGGATTTTTCAGCAGCAGGCCCATGTCCCTC
AACCCCAAACAAGCATTTAGATCATTATCTTTCTGTTTAAATCAAGAACG
CATTATTTAGCCTTTTATTTGGGGTTCAAGATACTCCTACAATGGTTCTA
AATCATAAGAAAAAGGGGCTTGATTTAAAACCCCTTGTTTTGGGCCAGGA
ATGGTGGCTCACGCCTGTAATCCCAGCACTTTGGGAGGCCAAGGTGGGCA
GATTACCTGAGGTCAGGAGTTCGAGACCAGCCTGGCCAACATGGTGAAAC
CCTGTCTCTACTAAAAATACAAAAATTAGCTGGGCATGGTGGCAGGTGTC
TGTAATCCCAGCTACTCGGGAGGCTGAGGCAGGAGAATCGCTTGAGCCCA
GAAGGCAGAGGTGAGCTGAGTGAGCTGAGATCGTGCCATTGCACTCCAGC
CTGGGCAAAAAGAGCAAGACTCCATCTCAGGAAAAAAAAAAAAAAAGAAA
ACAAAAAAAACCCTTTTTTGAGAAGAATTACGGAGCAAAGTAGAAAAATA
GTAGCTGGGTGTTAACATTAAATGCTGGATTTTTTTCATGGCTTGTCTTC
CCAATCATATTCCCTCAAATTGTGTTTCCTCCTCTGGTAACCCAGGTTGG
TTATGCTTAGCAAGTCCATGAACAATAAATATACATGGAAAACCTCCTGT
GTAGAATTGGTCAGACACCTAGATAAGATCCTTGCCCTAAAGCAGTTTAG
AAACCAGTTAGAAAGAAAGCAGAGTAAGGAAAACCACTAACAAAGCACGG
TATCAACTCAGTGGATAGTCAGCAAGTGAGCAGGGGGTCCAGGGACTGAC
AAAGCTGGGATGGGCAGGGAAGGCCTCTTGGGGGTAGGGTGTGAGTATGG
CCTTCTTACAAGCGTGTGATGTGTAGTAATTAAAATGCAGGAGGCCTAAT
GGGTGGGCAGCTTACATAGGAGTATAAACCAAGCTTGACCAGGAGCTGAA
AGGTTAAATGGTGGCTCTTAGGGGAAAACCCTATAAACAGTGGCTGAAGT
TCATTTATTCAACAAAGATATGAGTTCTTGTTTCTCATTTTTTGTTTTGT
ATTATTTTGTTTTGAGACAGGGTCTTACTCTGTCGCCCAGGCTGGAGTGT
AGTGGCTGGATCATAGCTCACTGCAGCCTCAAACTCCTGGGCTCAAGCCA
TCCTCCTTCTTCAGCCTCCACCTCCAGCTAATTTTTAAAAATATTTTGTA
GAGACAAGGGCTCACTTTGTTTCCCAGGCTGGTCTTGAACTTCTGGCTTC
AAGTGATCCTCCCGCTTCGGCCACCCAAAGTGCTGGGATTACAGGCGTGA
GCTGTAATTTAGTTGTTTATTTACTCATTTGTTCAACAAATACTTATTGA
ATATTTGCTCTTTGGCCAGTCAAGGGATTTCATGAGTGTCTACTATGTGA
ATAACACTGTGTTGGCCACTAGTCTGTCACCTACTGGTGGATTAGAAAAA
TAGCGCGAGGACCATTTTTTCTTTTCTTTTCTTTTTTTTTTTGAGACGGA
GTCTTGCTCTGTTGCCAGGCTGGAGTGCAGTGGCACAATCTCGGCTCACT
GCAACCTCCGCCTCCCGGGTTCAAGCGATTCCTCTGCCGCAGCCTCCCCA
GTAGCTGGGATTACAGGCAAGCGCCACCATGCCTGGCTAATTTTTTTGTA
TTTTAGTAGAGACGGGGTTTCACCTTGTTGGCAAGGATAGTCTCGATCTC
CCGACCTCGTGATCCACCCGCCTCGGCCTCCCAAAGTGCTGGGATTACAG
GCATAAGCCACCGCACCCGGCCAACTCTTTTCTTAAATTAGCCAGGGAGG
CGTGGGTGGGTTGGGTGAGGAGTTGGGTGGGGGGATCTCATTCAGTATTC
AAACTTCTACAAGTTTCGGGGTTGAGGTGGGTGATGGTAAGGGAACAGGC
CCTGCCACTACCTTTCATAGTGACTTCCATTTGTGTAATATTTTTGGTCC
ACTGAGAGCTATTATTTTATTTGATTCTTATGACCATCTTGTGAAGGAGT
ATCAACAGATACCCCGTTTTGATTTTATCAGATGCATGATTTGTCCTACA
TCAAACTTCATAAATGATGGACAGAATGGAGGAATCCTTCAGACCAAGTG
CTGCCTACTTCCCACCCCAATGGTGGCCTCAGCCTGGGCTCACATCACAC
GCCCCAAGGAGCCTTGGAAAAAATAAAGGCTCTTGGCTCCTTCCTGGGAC
AGCGTGATTCCTCATGTCTGAGCAGGCCCATGAACTTGTATTTTTCAGAC
GTTCCCTAGGACCCGTGTCCATCTGGATTAGGGAACCACTACATTATACC
ACTTCGCGGGAAGACTCAGGGGGAAGCATTTTAGCCACTTTCCTGTGTTC
CACAGTACTGGAGGGTGTTCTGAGTGGGCTGTGATTAATTTCCAAACCAA
CCACACGTCTCCCCTCAACTCCCACTGCTTACTCTTTGCTTCCTAGACAT
TCACTGCAGGCTGGAGACTTCTGGAAGCCAACAGCATCGCTGTAGAATTT
ACAGGGTCCAGTTCCCGGTGGACCACAAAACCTAAATTATGTGGCTGGGG
AAAGCTGAAATCCAAGGGAAGGGTTTGAGGAGGGGCTGACCTTATAATAA
AACCGGCTTGTATTTACTAAGTGTTAACTATGCGCTAGGCCCTCGTTGAC
GCCTCAACTCTATGTGAAAAGCACTATTATCCCCCATTTACAGATGGGAA
AACAGAGATTTAGAGCGCGAAAATCATTTCCCCAAGGCGCACAGACTCCA
AAGCCCACGCTACCAGGTACAACCTCAAGGCTGCGGCGTCTCTTCACCTG
CCCCCTAGCCCCCAAACCGCTGCTATGTCTAGGGCCTGACATTCCGGCGC
CCTCTGGGACGTGCTCAGATGCAGGGGCGCAAACGCCAAAGGAGACCAGG
CTGTAGGAAGAGAAGGGCAGAGCGCCGGACAGCTCGGCCCGCTCCCCGTC
CTTTGGGGCCGCGGCTGGGGAACTACAAGGCCCAGCAGGCAGCTGCAGGG
GGCGGAGGCGGAGGAGGGACCAGCGCGGGTGGGAGTGAGAGAGCGAGCCC
TCGCGCCCCGCCGGCGCATAGCGCTCGGAGCGCTCTTGCGGCCACAGGCG
CGGCGTCCTCGGCGGCGGGCGGCAGCTAGCGGGAGCCGGGACGCCGGTGC
AGCCGCAGCGCGCGGAGGAACCCGGGTGTGCCGGGAGCTGGGCGGCCACG
TCCGGACGGGACCGAGACCCCTCGTAGCGCATTGCGGCGACCTCGCCTTC
CCCGGCCGCGAGCGCGCCGCTGCTTGAAAAGCCGCGGAACCCAAGGACTT
TTCTCCGGTCCGAGCTCGGGGCGCCCCGCAGGGCGCACGGTACCCGTGCT
GCAGTCGGGCACGCCGCGGCGCCGGGGCCTCCGCAGGGCGATGGAGCCCG
GTCTGCAAGGAAAGTGAGGCGCCGCCGCTGCGTTCTGGAGGAGGGGGGCA
CAAGGTCTGGAGACCCCGGGTGGCGGACGGGAGCCCTCCCCCCGCCCCGC
CTCCGGGGCACCAGCTCCGGCTCCATTGTTCCCGCCCGGGCTGGAGGCGC
CGAGCACCGAGCGCCGCCGGGAGTCGAGCGCCGGCCGCGGAGCTCTTGCG
ACCCCGCCAGGACCCGAACAGAGCCCGGGGGCGGCGGGCCGGAGCCGGGG
ACGCGGGCACACGCCCGCTCGCACAAGCCACGGCGGACTCTCCCGAGGCG
GAACCTCCACGCCGAGCGAGGTAAGAGCCGCGGCGCCCCCGGATCTGGGG
CGGGCTTGGCGTCCCGAGCGGCCCCCGGCGCCGGAGCCTCCCGGCTGCGC
GCTTTGCCCGCCGCAGCCCAGCCGGGGCCGGCGCCTCCCTCCGCTCGCCG
CCCGCCCCTTTCACCTCCTGGCTCCCTCCCGGGCGATCCGCGCCCCTTGG
GTCTCCCCTCCCTTCCCTCCGTCCGCGTCTCCTGCGCCCCCTCCCTGCGC
TCGTCCCGCCGCTCTTCCCGCCGCCCAACTTTTCCTCCAACTCGCGCTCG
GGAGCTGGCGAGGCGGCGGCGGCTCCTCAGTGAGTCCCGGGAGGGACAGG
CCCGGGGCGAAGGCGGCGAGGCCCGCGGTTTCCTGGACTGGGGAGGAGGG
CGGGAGTGGGCGGCGAGGTGGGATGCGTTGTGTGTGTTATGTGTGTGTGT
TGCATTCCACTCCATGTCTTTTTGGTCCCCTTTTGGGGATTCACCCCCAA
TTCAGCAGGTAGCTTTGGGCTCAACGCTAAAAATCCGGGGCATTCCTAAG
TCCTTTTCCACCCCCGGGAAAGCCTGGGGTGCGGGTTGGGGTCGGATGGG
GTGGGAGATGAACTGCGGAGGACGTGGAGGGCTAGGTTAGCTTCTCTTGG
AATAGGTTTTAAGGAGGTGTCGTCACCAAATGGCTGAATCTGCTTGAGCT
GAGAGCGAAAAACGACTCCCCTTTCCAGAAGGGGTGATCTTATGACTTGG
ACGGTCTCTGAAAGGGTCGGAAGTTTGGGGAACGGGAGGACAACCCACGG
TCGTTAAGCCGAGGTGTGGGATGGGGGCGGAAGGACCGTTCGGTCCCAAT
CTGGTTCCTAGAGGTGGGGGAAGGGATGAGGGTTTTTGTCCGGTGTGGTT
CACTCGGCAGCGATGCGTATGCTTCTCTGGCCCAGACCCCTCTGCACCTC
GCTTCCCCTACCGTTATGTTTGGGGTTGGGAGAAAAGTGAGGCTACGACC
CATGTTTGCGGAGGAATTTTATGGACCTTGTAGATGGGGGTTCATATAGA
ACACACACCCCCTATGAGGCAGCCAGACACTTTTTTGGTGGTGGTGGGGG
GGGGGTGGGGTGTGAAGCCTGTTTCTTGTTCTGAGCCCAGAAGCTATCAA
CCCTTTTGAAAAACATTACCACGGTGCCTTTCTCCCCCAGCACTCCCCCA
CCCCCAATTTCCAGATGTAGCAGCCGCATCTGGTTCCGTTTCACCCCACA
CGGGTACACCGCAGCCGCATTATTAACTTCCCTCTTCCTCCCCTCCCCCT
CCCCCAAATTAAAACTCAGATTCTTCAGCCTGTCTTGACCACCTCCCTCC
TTAACATTTCTGGAGACTTGGAGATGCGGCGTTGAGATTCGGGGGAGAAA
AGAAAGTTCCCTTGGATCCCGAGTTATTTAAGATCTCACCAAGTTATTCG
CCGCCGCTGGTGGGTGGCGGCGGTCCGGGTGCTTTCTGGATTGCGCAGTA
AAGAGGCATCTTGGGAGATGGGGCCAAGGTTTTAGGGGGTGCCACTCGCG
AACGGTTCATCCGCTAGACTAGGGGGGCTCTTTGGCTGTGCGTCTGGCCA
GAACTGGCCTTGACGATGGAAGTTTCTGGAACCAAAGCGTTGCTTTCTCT
CCCTTGTGTTATAGCTGGAGCTGCGGGAGCGCCTGCCCTGCCCGGAGCCC
GCGGTCCCCTCTCGGCTGCCCCGCGGTGGCGTCACGCGCCCCTCCCGGAG
CAAGCCCGGTGCGCAGGGCCGGGGGCGTGGGCGGCTGCTGCCAGAGGCGC
TCTCTGTGTGTTTTTAAGGACTGATTTGGGCCGCATCCCCCGGAAACTAA
AGTGGGGTGTTTTACCGTTTAAATAACGGCTACAGGTTTGAAAGCGGGGT
TGGATTTTCGAGTTGTGTTTGGTAATAGTCTTTGAGGCAGGAAAGCGCCT
TGTGGTCCAAAGTTGCCGGGAGGGTGGGGAGAGTCGGTGTCTTACCCGCT
TCTTTCCAGCCTCTTTCAAATTGAAAACACTTCTCTGGTTTCCTTCTTTG
GGCGGTAGTTTTGGAGGCTGTAATGAAATCGCACTTTCTCTAGACGTGGT
AATTAAGGTGACTGTTTCCTCCGCAGATGTGCCCTACCCTTTGCACCTCC
GGACCAGCGCTTTTTTTGGAATACTATCTAGCCTTGAGACTGTTTAGCAG
AAAGTGGCCATTTTCCTCCCTTGGCCCGGGCTCCCGGTTTCCTCCCTGAG
GCTTGTTTAAAAGCGAAGTAGCAGGGCCCCGTGGGACGCGCCTTGGTCTG
GGTAATCACCCCCACGCCCGGGTCATCCACCTTCCTCTCGGTGACCGAGG
TTCAGCAGCCTCTGCTATTGCCGGCCGTCTTTGCCGATGGCCTGCCTCCC
TAATGACTTGTTTACATATCCTACCCCCAGTGGGTTAGGAGAAGCTCCGG
GGCTGCCCCGACCCTCCGAGTGCAGGGTGTTTGGGGACCGGGAGGCTGCT
GGGGCCTGACTCCAGCTGGGAGGGTTATGAACTGCATCAGTGACGAGCTG
CTTGAAATATCTGTTGCATTTACTCTTAGTCATAGCTGAGTGTCAGCTTT
TTAATGAGGTTCATCCAGATTGAGAGCCACTTGGACTGCGTACTTCACTG
CCTGCTTTTCCAAACATGCCTGCAGAAATGCTCATTTTCGAGGTATTTTT
CCCAATGGGAATTCAGGCCAGAGTGGGCACCACTTGAACAATCTTAGGGT
GCTTCTTTTCCTTGGCCTCTGGCCATGGAGGGTGTTAGACAGTTCCATTA
GGTGGCCCTTTGATAGCAAGGGAAGCAAAGGCTCAGGAAGAAATGGAGAA
GCGTCCCCCACTCCCTAGGGGCAGAGGATTAGATACATCGGTGCATCCCT
CAGGCTGGGCTAGCTTTATTCCTGGTGGACTCCAGAGGGCAAGAAAATTG
AATTGAACACTGGGTAGGCAGATTCAAGCCTTAGAGACCAAGGAAAATCC
ATGGGTTTTGCTTTTAGTGGTGTGCTCTTTGTTTTCAGTATTGACCTGAA
ACAAGACTCCTAAAATGAGAGATTTGCTGGTATGAACTTGGGGGTTTAGC
AGCCGGCTTCTACAAAGGCTTTTTTCTTGCCTTCGTTTCTAAAGTGTCTT
TCGTCAAAATGGCTGTTAGTTATAGAACATCCTAGCAAAGTTTGAGCCTG
TTGCTGCTGGAGGAAAAGGAGTTAGAATTGATTCAAATGTCTTATTCTGA
AAGGGCCTCACATCACTTGATAGTTTAATTTCCTCCTGGGAAATTTGTGT
CTTACATTTGTCTTCCCCAGAGCTTTGTAAAAGGCCTGAACGCACCAGGG
ACTAGTGGGAGCCCAGATGCAGAGCTTTAGAGAAGATTCTGGTGTTTCCA
GAGAGGATGAAATGTCAGACTTGGGCTAGGATATTTGTTTTTCCTCCTAA
GGTTGCATCTACTTTAAACAGAAATTCTCTCCTCGCCACCATTTATCTCT
CCCCTGCAATGAAAGAAACCATGTTTAGGGCCCTCTCCCCCATTTAATAG
CCCTCACATGGATGAACTATCCCAAGAATTTGGTGGGGTTCCACTCATAG
TACATCCTGTCTTCAAGAGCAAGGTTTTCTAGATTATGTGCAGCAGTTCG
TGTTTCACTTGTTGCTTTTTTTTTTTTTTTTTTTTTTTGAGATAGTCTCG
CTCTGTCGCCCAGGCTGGAGTGCTGTGGCGCTATCTCAGGTCACTGCAAC
CTCCGCCTTCCGGTTGAAGCGATTCTCCTGCCCCAGCCTCCCTAGTAGCT
GGGATTGCAAGCATGCGCCACCATGTCCGGCTAATTTTTTGTGTTTTTAA
TAGAGATGGTGTTTCACCATGTTGGCCAGGCTGGGCTTGAACTCCTGACC
TCAAGCAATCCGCTGGCCTCGGCCTCCCAAAATGCTGGGATTACAGGTGT
GAGCCATTGTGCCTGACCACTTATTGCTAATTTTTTATATGTCTCTTACT
TCCAAGGACATTTAGACACTTTTTTTTTTTAAAGAGACTCAAAAAATTAG
CATTTCCATTGGACCAACTAAAATTTAGCAAGCTGAGCTGAGTAACTTTC
TCCATATGTTTATTAAGTACTTGCCCCCTGCCCTCTCAACATGTGAGTAG
AGAATGGTCACTTTGGGGAAGAAATAAGTCTTATTCTCATCTGAAGGGAT
TAATGTTTTGGTGTTACTTCCTCAATTCTGAAGAACCAAGTTGTCCAGAA
ATTTTCTCAGGGTTCTTTGGACTAGAGTTTGGCTGGTTAACAAGGGGTAC
TACCTAATTGCTTTTCTCTGATATTCTCAGCCTCTTTTTCTGGAGGAGTA
TCTCTGTCAGTTTCTTTTCATCAGCCCTTTTTTTTCCTTCATTCACTTAC
TCATTCATCCAGTTAACAAACATGTTGGCATCTCCTGTGTACATGCTAGG
TGCCGAGGGTGTTAGCAAAGGTTAGGGAGGCACAGACCCTGTTCTGAAGG
AGCCTGCAGTTTCGTGGGGAGAGAAGAGAATGAAGAACATAAATAACAAT
CATATAATATGACCTAAGTGCTATGTGAGAGGGGCTAGTAATGTGGTTTG
CAAATTTGGAGGAATGAAATTCTCCAGCTAGAAGGCCCAAGAAAGTCTTA
TGGAAGAAACAGCTTCTTAAGGTGGGGTTCAGAGAAAAGGGAAGGGCTGG
CCTGTTGCAGAACAAGGAATGGCATGAAGAAAGTCTTGCACAGAGGCATG
GATGTTGCTTCGAGCTGTGGCGCCCTATAGAAATAGAACATGAGCAGCTG
GTCACAGTGGCTCATGCCTGTAATCCCAGCACTTTGGGAGGCCAAGGCAG
GCGGATTGCTTGAGCCCATGAGATGGAGATGAGCCTGGACAACATGGTGA
GACCCTGTGTCTACCAAAAAATACACAAATTAGATGAGTATGCTCGTGCT
TACTGGTAGTCCCGGCTATTCAGGAGGCTGAGGTGGGAGGATCACTTGAG
CCTAGGAGGCAGAGGCTGCAATAAGCTGTGATTGCACCACTGCATTCCAG
CCTGGGGGACAGAGGAAGACCCTGTTTAAAAAAAAAAAAAAAAAAAAAGC
CAGGCACAGTGGCTCATGCCTGTAATCCCAGCCCTTTGGGAGGCCAAGGC
AGGTGGATCACCTGAGGTCAGGAGTTCAAGACCAGCCTGGCCAACATGGT
GAAACCCTATTTCTACTAAAAATAAAAAAATTAGCCGGGCTTGGTGGCTC
ATGTCTGTAATCCCAGCTACTTGGGAGGCAGGAGAATCGTTTGAACCCGG
GAGGCGGTGGTTGAGCCAAGATTGCGCCACTGCAACTCCAGCCTGAGTGA
CAGAGCAAGACTCCATCTCAAAGAAAAAAAGAAAGGAAGAAAGAAATATA
ACATTATAACATGAGTTATGTATATGTTCAGATTTTCTAGAAGCCACATT
GGAAATTAAGTTAAAAGAAAGAAATAGGTAAAAAAAATTTTTTTTTTTGA
GACGGAGTCTCACTTTGTTGCCAGGCTGGAGTGCAGTGGCGCAATCTCGG
CTCACTGCAACCTCTGCCTCCCGGGTTCAAGCAATTCTCCTGCCTCAGCC
TCCTGAGTAGCTGGGACTACAGGCGCGCGCCACTGCACGCAGCTAATTTT
TGTACTTTTAGTAGAGACGGGGTTTCACCATGTTGGCCAGGATGGTGTCG
ACCTCTTGACCTCGTGATTTGCCCACCTCAGCCTCCCAAAGTGCTGGGAT
TACAGGCGTGAGCCACCGCGCCTGGCCAATATTTGTTTTTTAATTAACTT
GTTTGTTTAGATTTTATTTAATGTAACTATATTTCCAAAATATTATCATT
TGAACATGTAATCAATATAGAAATTATTGATGAGATACTTTACATTTTTT
TCATAACAAGTTTTTAAGATGCGGTGTATACTTTTTACTTATAGCATATC
CGTTAGCACCAGCCACATTTCAAGTGTGCAGTGGCCACTGTGTGGGCCAC
AGGTCTAGAATATAAGACATGAAGATGGAGAGTGAGAAATGCCTTTGGAA
AGGTTGGAAGTTCCTGTCCTTCTGCTGCCAATTACCAAATCTCCTGAGAG
TGCTATTAAGGAGTGACTCAAAGCACTACACAAAGAGAATTATAAATATC
TTAATATTATATCTGAAATCCAAATGCATAATTCTTTACATTTGGTTGGT
ACTTTAGAGAGGAGAGAATGGGCACAGTCACCCACACCACCCATTTGAGC
CTCATAATCACCTGTGATGTGGCTTCCTCTAGGTGGGAAACCGAGGCTTA
GAACGGTTAAGTGACTATCCCAGGGTGGCAAGATCATAAGTGGAAGGGTG
TGAATTCATACTGTCTCCAGCGGACAAGAATAAAAAGACCCAGGCTGGGT
GTGGTGGCTCATGCCTGTAATCCCAGCACTTTGGGAGGCCACTGTAGGTG
GATCTCCTGAGCCCAGGAGTTCATTACCAGCATGGGCAACATGGTGAGAC
CCCATTTTTATTAAATATACAGAAAATTAGCCCAGCTTCTCGGGAGGCTG
AGGTGGGAGGATCACTTGAGTCTGGGGGATGGAGGTTGTAGTGAGTTGAG
ATCGTGCCACTGCACTCTAGCTTGGGTGACAGAGCAACACTCTGTCTCAG
AAAGAATAAAAAGATTTGGCCATGAATTCGTCAGCTAGTTTTCCTTACAT
AATTTTTGGACAAGGAGATCTGACATTCATAGGTTTTTCTCTTAGAAGTG
GGAGAGCTTCAAGGTCACGTGGTCCGTCCAGCCCCTGCTATCTCACCAGA
CACTGTCCACCCTGTATGTTGGATCAGTACTCCAGTGAGAAGACAGCAGG
CACTTTCACCCATGCAGCCCATTCAGTCTTCATAACCACCTGTGATGGAG
GCAAGGCAAGTATTTCAGCCCCCTCTGATGAGTGGGAAACTGAGATGTGC
CCCCTCTCTGCTCCCCACCGAGGACCTCTGCATGCAGGCATGAATCCCAG
GAGCCTAGCTGATATTGGAGAGACGGGGCGGGGGGAACCAGCTGCAGGGT
CTTGGAGGAAGCTGCTGTGTACACCTGCAAGGCTGCAGGTTACATCTATC
TGTCAAGCAGTGAAGGAAGGAAGTTGTTTCTAAGGGATTGGAAAAATTCA
TTAATTAGTAGAATGAGAAACTGAGGTGAAGCAGGAGGTGGCAGGGTCCC
AGACAGCATGTTGGACTAGTGGCCTGTGTCACTGTGTTTTTTGCAGGCGG
GTGGCATGGGGTGTATGCTGACTTCTTATTCCAGGAGTTGGTGCCAGGAG
GCCAGGTTTTCTTAACATCCTTGTTTTACAGATGTCAAACTTGAGGGCCA
GAGGGGTAGGAGAGGAAGAGACTTTTTGTACCTTTTTTGGGAAAGAACAA
GAGGGAAGCTGGCAGATGAATTTGAAGTGCATTGACCAGGGAGCTGAGAG
AGGGCGGTCTGCAGCCAGCCCACACCTGGGAGGAGGCGGCAGGCTTTTCT
GGGGACAGAGTGGCCAAGTCGAAGCAAGCTTAACCATCTCAACATGACAC
CACTCTTTCCCATTGGAACCTGAGAACTTGTTCAGTATTCTGACACTTAG
CAAGGGACCTGGGTTTTCTTGGTCAGGTGTGCGTTTCTGGGTGACAGGCC
TGCATCAGGTGTATTTTCGGGATGTAGTAAGTTGTGGAATATGGGTTTAG
GGGCATCCTCTGGCAAGCACTGCTTCTATCCCAGCTCTGGGAATGTGCCC
CATGCAGTGTCCTAGATGGCCCATCTGTGGTCTGCTTCCAAGGGTCTTTC
TTTTAGTTAGTTAGTTTTGAGACAGAGTCTCACTCCGTCACCCAGGCTGG
AGTGCAGTGATGCAATCTCGGCTCACTGCAACCTCCACCTCCCAAATTCA
AGCAATTCTCATGCGTTAGCCTCCTGAGTAGCTGGGATTACAGGCGTGCA
CCACCACACCCAGCTAATTTTTGTATTTTTAGTAGACGAGGAATTTCACC
ATGTTGGCCAGTCTGGTCTCAACTCCCCACCTCAGGTGATACTCCCGCCT
CAGCCTCCCAAAGTCCCGGGATTGTAGGTATGAGCCAACATGCCCTGGCA
CAAGGGTCTATCTTTGACCAATGGAACTGCAAATCAAGCCTCTTTTGTTA
CCAGAGTTACCTTGGATTTACCCTTATCTACTTGGTTTGGATAAATTGAG
TTTGCATCAGATGGAGTCAGGCTTGATCAATCCCTTATTTACTTCCTCCC
ACCCTGTTCTCTAATATCCAAAAACCTTGAGGCACTATTACATGCTAGCT
ACATTTCCTTGAGTAAAGTACTTAACCTCTTTGAGCCTCAGTTTCTCCAT
TGCATAAAAGGAATAATAAAACTTATCCCCCATAAGTTTATAGTGAGGAA
TGAATTAATTCCTCACTATAGTTCTAAATTAATTCTACTTAGGGCATCCT
TGGTACATAGTGGGTGTTCAGTATTCATTTCATTTTCTCTTTTCTGATTC
CTTTCGTAAAAGTAGAAAAATGAAAGAGAAATGTTGACTTCTCTTTTGAT
TTGAAATCATTAAAACATTTTAGTAAGCCTTGGGAGGGAGCTAGTGGTGT
GGCATGTGTATCCCGCTGGCCAAGCACATGTGAACGAAGCCAAGAATCCA
GGGGCTTTTCTGCCAGCCAGCACTGACTCACTTGCGAGGGGCCCTGCCTG
GCTCGGGGGAGGCAGGGCTGAAGTACCACATTAGGGCATGTTCCGGGGAA
GTAGATTCTCTGAATAACTTGGATGGCTCCCTGGAGCATTTAGGACAGAA
GCCACCTGGAAAATAGAGATGGTCACCCCCACGTAGCCTTGACAGTGCCC
AGAAAGTCTTGTCACTTGGTAAATGTTAACAGCTATGATCCGTTCTTTAA
GACCCTGGGGAGTTTTAAGTTTTACCCCACCAGACCTGAGAAGGGTAAAG
GGCTGCAGATTCTGTTCTTTTAACTGGGGCCAGTGTGAGCCATCTTTGAC
TCAGTGCTTGCAATAGACCTTGATTCTGCAGTGGGACCTCCCAGGCCCCC
TTGCCCCCCGCAACTTCTGGACTCATATCCGTCAGACATCACTTGTCACC
TTCCAGCATCAGGGAGAACTGGATCCCTCCTGGCTCCACACTCTTAGGCT
CTTTGTAAGTAGCTGGTGAGGGTTTTCTTCTCTCTGCAAGGGAGGCTGGT
AGAACTATGGATGTGATTCGTACAATTTTAGAGACAAAAAGAAAGTACCC
AGGAGGTCATTTATTTCAGCTGCTTCATTGCATAGGTCGGGGAGTTGAGC
ATGGAGTCCAGCAGCTACTAACTAGTTATCTCTGTACCTGGCTTCCATTT
ACTGGTCCTTAGCTTGTTCCGTGATTCTTCATTGCCCCTTATTTCTCACC
AGAGGGACTGGTTGGCCCTAGATGGAGTGGTCTTTTTAAAATTTTTTTTT
TAAATTTTTTGAGACAGAGTCTCACTCTGTCACCTAGGCTGTAGTGCAGT
GCTGCGATCTCGGCTCACTGCAACCTCCGCCTCCTGAGTTCAAGCAATTC
TCCTGTCTCAGCCTCCTGAGTAGCTGGGATTACAGGTGTGTACCACTATG
CCCAGCTAATTTTTGTATTTTTAGTAGAGATGGGATTTCACCATATTGGC
CAGGTTGGTCTTGAACTCCTGACCTCAAATGATCTGCCCACCTTAGCCTC
CCGAAGTGCTGGGATTGCAGGTGTGAGCCACCGCACCTGGCCTGGGCAGA
GTGAAGTCTTATGCTGGGGAGCCATCAGCATGCTCAAACCTCCTGCAATT
GTAGCACACTTTGTAAAACTGTTTCCCACAAAAGGGCAGAACTATTTGGG
ACTTTCATGAGACCATTCACTTTGTAGCACATACTACTTTGAAGTTTATA
CCTTGGAAAACCTCATGATGGTATTCCCAGGCTTGCACGTAATCTGCACT
CAAAACATAGCTGTAGAATTGAACTAAAGCATCCCTCTGTCCAATTAAGA
CCTATAACCTCTCTTTTTGAGACAGAATCTCGCTCTGTCACCCAGGTTGG
AGTGCAGTGGTGCAATCTCAGCTCACTGCATCCTTCGCCTCCTGGATTCA
AGCGATTCTCTTGCCTTAGCCTCCGAAGTAACTGGGACTACAGGTGCGCG
CCACCACGCCTGGGTAATTTTTGTATTTTTAGTAGAGACGGGGTTTCGCC
ATGGCCAGGCTGGTCTCAAACTCCTGGCCTCAAGTGATCCTCCCGCCTCA
GCCTCCCAAAGTGCTGGGATTACAGGGTGCACCACCACACCCAGCCAGGA
CCTATGATCTAATTCATTGTTGGGGTAGCTTCACAATTTTCTTCTGGACG
CCTTAGTAAGTCCACACTTTAAGCAGCCACCACATGGCATACTTTACCTT
CTGTTTTTCCTTTCCCCTCCCCTACCTAGACCCTCCTAACTTTTGGGGTT
TTTTTCCTTTCCTCAGGGTCAGTTTGAAAAGGAGGATCGAGCTCACTGTG
GAGTATCCATGGAGATGTGGAGCCTTGTCACCAACCTCTAACTGCAGAAC
TGGGATG

III. DNA Methylation

In some embodiments, the present invention provides using oligonucleotide that are methylated at specific sites for screening purposes. The present invention is not limited to a particular mechanism. Indeed, an understanding of the mechanism is not necessary to practice the present invention. Nonetheless, it is contemplated that one mechanism for the regulation of gene activity is methylation of cytosine residues in DNA. 5-methylcytosine (5-MeC) is the only naturally occurring modified base detected in DNA (Ehrlick et al., Science 212:1350-1357 (1981)). Although not all genes are regulated by methylation, hypomethylation at specific sites or in specific regions in a number of genes is correlated with active transcription (Doerfler, Annu Rev. Biochem. 52:93-124 [1984]; Christman, Curr. Top. Microbiol. Immunol. 108:49-78 [1988]; Cedar, Cell 34:5503-5513 [1988]). DNA methylation in vitro can prevent efficient transcription of genes in a cell-free system or transient expression of transfected genes. Methylation of C residues in some specific cis-regulatory regions can also block or enhance binding of transcriptional factors or repressors (Doerfler, supra; Christman, supra; Cedar, Cell 34:5503-5513 (1988); Tate et al., Curr. Opin. Genet. Dev. 3:225-231 [1993]; Christman et al., Virus Strategies, eds. Doerfler, W. & Bohm, P. (VCH, Weinheim, N.Y.) pp. 319-333 [1993]).

Disruption of normal patterns of DNA methylation has been linked to the development of cancer (Christman et al., Proc. Natl. Acad. Sci. USA 92:7347-7351 [1995]). The 5-MeC content of DNA from tumors and tumor derived cell lines is generally lower than normal tissues (Jones et al., Adv. Cancer Res 40:1-30 [1983]). Hypomethylation of specific oncogenes such as c-myc, c-Ki-ras and c-Ha-ras has been detected in a variety of human and animal tumors (Nambu et al., Jpn. J. Cancer (Gann) 78:696-704 [1987]; Feinberg et al., Biochem. Biophys. Res. Commun. 111:47-54 [1983]; Cheah et al., JNCI73:1057-1063 [1984]; Bhave et al., Carcinogenesis (Lond) 9:343-348 [1988]. In one of the best studied examples of human tumor progression, it has been shown that hypomethylation of DNA is an early event in development of colon cancer (Goetz et al., Science 228:187-290 [1985]). Interference with methylation in vivo can lead to tumor formation. Feeding of methylation inhibitors such as L-methionine or 5-azacytodine or severe deficiency of 5-adenosine methionine through feeding of a diet depleted of lipotropes has been reported to induce formation of liver tumors in rats (Wainfan et al., Cancer Res. 52:2071s-2077s [1992]). Studies show that extreme lipotrope deficient diets can cause loss of methyl groups at specific sites in genes such as c-myc, ras and c-fos (Dizik et al., Carcinogenesis 12:1307-1312 [1991]). Hypomethylation occurs despite the presence of elevated levels of DNA MTase activity (Wainfan et al., Cancer Res. 49:4094-4097 [1989]). Genes required for sustained active proliferation become inactive as methylated during differentiation and tissue specific genes become hypomethylated and are active. Hypomethylation can then shift the balance between the two states. In some embodiment, the present invention thus takes advantage of this naturally occurring phenomena, to provide compositions and methods for site specific methylation of specific gene promoters, thereby preventing transcription and hence translation of certain genes. In other embodiments, the present invention provides methods and compositions for upregulating the expression of a gene of interest (e.g., a tumor suppressor gene) by altering the gene's methylation patterns.

The present invention describes the use of unmodified completely complementary DNA oligonucleotide sequences to inhibit gene expression. The present invention is not limited to the use of methylated oligonucleotides or modified oligonucleotides to identify therapeutic sequences. We describe the use of non-methylated oligonucleotides for the inhibition of gene expression and we prove this system works by providing the results of experiments conducted during the course of development of the present invention. For example we demonstrate that an unmethylated oligonucleotide targeted toward Bcl-2 inhibited the growth of lymphoma cells to a level that was comparable to that of a methylated oligonucleotide.

IV. Oligonucleotides

The term “oligonucleotide,” refers to a short length of single-stranded polynucleotide chain. Oligonucleotides are typically less than 200 residues long (e.g., between 8 and 100), however, as used herein, the term is also intended to encompass longer polynucleotide chains (e.g., as large as 5000 residues). Oligonucleotides are often referred to by their length. For example a 24 residue or base oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

In some embodiments, the present invention provides DNAi oligonucleotides for inhibiting the expression of oncogenes. Exemplary design and production strategies for DNA is are described below. The below description is not intended to limit the scope of DNAi compounds suitable for use in the present invention. One skilled in the relevant recognizes that additional DNA is are within the scope of the present invention.

A. Oligonucleotide Design

In some embodiments, oligonucleotides are designed based on preferred design criteria. Such oligonucleotides can then be tested for efficacy using the methods disclosed herein. For example, in some embodiments, the oligonucleotides are methylated at least one, preferably at least two, and even more preferably, all of the CpG islands. In other embodiments, the oligonucleotides contain no methylation. The present invention is not limited to a particular mechanism. Indeed, an understanding of the mechanism is not necessary to practice the present invention. Nonetheless, it is contemplated that preferred oligonucleotides are those that have at least a 40% CG content and at least 1 CG dinucleotides. In some embodiments, oligonucleotides are designed with at least 1 A or T to minimize self hybridization. In some embodiments, commercially available computer programs are used to survey oligonucleotides for the ability to self hybridize. Preferred oligonucleotides are at least 10, and preferably at least 15 nucleotides and no more than 100 nucleotides in length. Particularly preferred oligonucleotides are 20-34 nucleotides in length. In some embodiments, oligonucleotides comprise the universal protein binding sequences CCGCCC and CGCG or the complements thereof. In some embodiments, oligonucleotides comprise the universal protein binding sequences (G/T)CCCGCCC(G) and the complements thereof. It is also preferred that the oligonucleotide hybridize to a promoter region of a gene upstream from the TATA box of the promoter. It is also preferred that oligonucleotide compounds are not completely homologous to other regions of the human genome. The homology of the oligonucleotide compounds of the present invention to other regions of the genome can be determined using available search tools (e.g., BLAST, available at the Internet site of NCBI).

In some embodiments, oligonucleotides are designed to hybridize to regions of the promoter region of an oncogene known to be bound by proteins (e.g., transcription factors). Exemplary oligonucleotide compounds of the present invention are shown in Table 3. The present invention is not limited to the oligonucleotides described herein. Other suitable oligonucleotides may be identified (e.g., using the criteria described above). Exemplary oligonucleotide variants of the disclosed oligonucleotides can include smaller oligonucleotide sequences of 20-mer or can be right or left shifted 20 base pairs. Candidate oligonucleotides may be tested for efficacy using any suitable method, including, but not limited to, those described in the illustrative examples below. Using the in vitro assay described below in the material and methods and Figures, candidate oligonucleotides can be evaluated for their ability to prevent cell proliferation or target inhibition at a variety of concentrations. Particularly preferred oligonucleotides are those that inhibit gene expression of target proteins as a low concentration (e.g., less that 20 μM, and preferably, less than or equal to 10 μM in the in vitro assays disclosed herein).

B. Materials and Methods

### Oligonucleotide Preparation (FIGS. 1-25, 27-30, 31-49, 54-67)

All oligonucleotides were synthesized utilizing cyanoethyl phosphoramidite chemistry, purified by reverse phase high-performance liquid chromatography (RP-HPLC), and lyophilized by The Midland Certified Reagent Company (Midland, Tex.). Methylated oligonucleotides were methylated at all CpG sites.

### Cell Culture (FIGS. 1-25, 27-30, 31-49, 54-67)

Human lung carcinoma cells (A549; ATCC) were cultivated in DMEM medium (ATCC) containing 10% fetal bovine serum (FBS; Invitrogen) and maintained under a humidified atmosphere of 5% CO2 at 37° C. Cells were split 1:8 at 90% confluence and used for experiments between passages 12 and 20 (2,500 cells per well were plated 12-24 hours prior to adding oligonucleotides).

Human breast carcinoma cells (MDA-MB-231; ATCC) were cultivated in Leibovitz's L-15 medium (ATCC) containing 10% fetal bovine serum (FBS; Invitrogen) and maintained under a humidified atmosphere at 37° C. Cells were split 1:6 at 90% confluence and used for experiments between passages 15 and 22 (2,500 cells per well were plated 12-24 hours prior to adding oligonucleotides).

Human prostate carcinoma cells (DU145; ATCC) were cultivated in EMEM medium (ATCC) containing 10% fetal bovine serum (FBS; Invitrogen) and maintained under a humidified atmosphere of 5% CO2 at 37° C. Cells are split 1:8 at 90% confluence and used for experiments between passages 10 and 16 (2,500 cells per well were plated 12-24 hours prior to adding oligonucleotides).

Human breast carcinoma cells (MCF-7; ATCC) were cultivated in 50:50 RPMI/DMEM medium (ATCC) containing 10% fetal bovine serum (FBS; Corning), 0.01 mg/mL insulin (Sigma-Aldrich) and maintained under a humidified atmosphere at 37° C. at 5% CO2. Cells were split 1:6 at 90% confluence and used for experiments between passages 15 and 18 (2,500 cells per well were plated 12-24 hours prior to adding oligonucleotides).

Human colorectal carcinoma cells (HCT-116; ATCC) were cultivated in McCoy's 5A medium (Corning) containing 10% fetal bovine serum (FBS; Corning) and maintained under a humidified atmosphere at 37° C. at 5% CO2. Cells were split 1:6 at 90% confluence and used for experiments between passages 4 and 7 (2,500 cells per well were plated 12-24 hours prior to adding oligonucleotides).

HepG2 cells were plated using 5,000 cells per well in 96 well plate (for both qPCR experiment and cell count experiments). Cells were incubated for 24 hours prior to treatment with DNAi oligonucleotides. Twenty-four hours after plating DNAi oligonucleotides were added to the cells at final concentration of 15 uM. At each timepoint (24, 72, and 144 hours) cells from 96 well plate were washed with 1×PBS once and total RNA isolated using MagMax-96 Total RNA isolation kit (Lifetech, cat#AM1830). At 72 hour timepoint cells were over 90% confluent, therefore cells were washed with 1×PBS twice, trypsinized with 0.05% Trypsin-EDTA and transferred from each individual well (96-well plate) into 24-well plate. STAT3 DNAi oligonucleotides were added to the cells in 24-well plate at final concentration of 15 uM.

HepG2 cells were trypsinized (as described above) and cells from each well (96-well plate) were diluted in 1 mL of complete growth medium prior to cell counting performed using Guava PCA-96 flow cytometry system. HepG2 cell culture work was performed at Altogen Labs (Austin, Tex.).

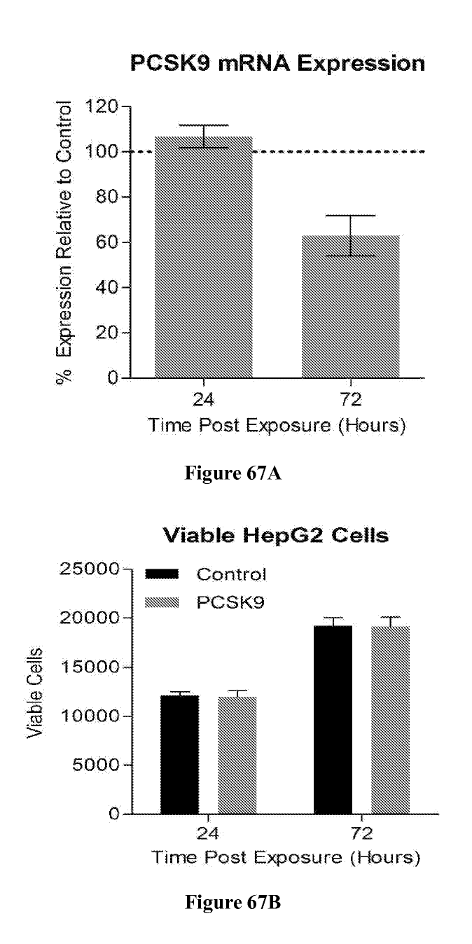

mRNA Expression Analysis and RNA Isolation (FIG. 67)

All RNA was isolated using the MAGMAX96 Total RNA Isolation kit (cat#AM1830; Lifetech). The manufacturer's protocol was followed, including a final elution of 50 μL elution solution. RNA was stored at −20° C. for later use.

### Reverse Transcription (RT) (FIG. 67)

Isolated RNA was reverse transcribed into cDNA in a single reaction containing RNase Inhibitor Protein (15518; Lifetech) and MMLV-Reverse Transcriptase (18057; Lifetech). RNA input into the RT reaction was based on a 7.5 μL input per 20 μL reaction size for all samples.

qPCR (FIG. 67)

Fluorescence based, real-time reverse transcription-PCR (qRT-PCR) is a standard tool used for quantification of mRNA levels. This technique has high throughput capabilities with both high sensitivity and specificity for the target of interest. The amplification reaction consisted of dNTPs (PCR grade; Roche) and Platinum Taq Polymerase (10966; Lifetech). Cycling conditions were as follows: 95° C. for 1 minute; then 50 cycles of 95° C. for 5 seconds and 60° C. for 20 seconds. Results were determined by real-time PCR on the ABI Prism 7900 SDS real-time PCR machine (Applied Biosystems, Foster City, Calif.). All qPCR work was performed at Altogen Labs (Austin, Tex.).

As shown in FIG. 67, PC2 (206; exposed at 15 μM), a PCSK9 targeted oligonucleotide, demonstrated an approximate 40% decrease of PCSK9 mRNA at 72 hours post-exposure compared to control PCSK9 mRNA levels in HepG2 cells. While PC2 (206) decreased PCSK9 mRNA expression, it was not cytotoxic to cells at either 24 or 72 hours post-exposure in the same experiment. This demonstrates that an oligonucleotide is capable of modulating target gene expression with expected phenotypic changes.

Altogen Labs (Austin, Tex.) performed the cell culture work for A549, MDA-MB-231, DU145 and START Preclinical (San Antonio, Tex.) performed the cell culture work for MCF-7 and HCT-116.

### Cell Growth Inhibition Assay (FIGS. 1-25, 27-30, 31-49, 54-66)

Cells were harvested from T-75 flask by a single wash with 1×PBS and incubation with 2 ml of 0.05% Trypsin-EDTA (Invitrogen) for 7 minutes at 37□C. Trypsin was inactivated by addition of 8 ml of complete medium (total volume of 10 ml). Cells were counted using hemocytometer and cell count confirmed by Guava PCA flow cytometry. Cells were then plated and assayed. Cell growth inhibition was assessed using a Vybrant MTT (3-(4,5-dimethylthiazol-2-yl)-2,5-diphenyltetrazolium bromide) Cell Proliferation Assay (cat#V13154) purchased from Life Technologies (Carlsbad, Calif.). For each cell line 2,500 cells per well were plated 12 hours prior to adding oligonucleotides. Absorbance measurements at 570 nm were made using a Molecular Devices Spectramax Plus (Sunnyvale, Calif.) microplate reader. Each treatment was run in quadruplicate. Altogen Labs (Austin, Tex.) and START Preclinical (San Antonio, Tex.) performed the cell growth inhibition assay. Included in Tables 4 and 5 are the sequences for the control and negative control oligonucleotides used in the experiments.

Oligonucleotide Preparation (FIGS. 26, 50-53; Descriptions Referenced in U.S. Pat. No. 7,524,827)

All oligonucleotides were synthesized, gel purified anal lyophilized by BIOSYNTHESIS (Lewisville, Tex.) or Qiagen (Valencia, Calif.). Methylated oligonucleotides were methylated at all CpG sites. Methylated Oligonucleotides were dissolved in pure sterile water (Gibco, Invitrogen Corporation) and used to treat cells in culture.

Cell Culture (FIGS. 26, 50-53; Descriptions Referenced in U.S. Pat. No. 7,524,827)

Human breast cancer cells, MCF7 and MDA-MB-231, were obtained from Karmanos Cancer Institute. All cells were cultured in DMEM/F12 media (Gibco, Md.) supplemented with 10 mM HEPES, 29 mM sodium bicarbonate, penicillin (100 units/ml) and streptomycin (100 μg/ml). In addition, 10% calf serum, 10 μg/ml insulin (Sigma Chemical, St Louis, Mo.), and 0.5 nM estradiol was used in MCF7 media and 10% fetal calf serum was used for MDA-MB 231. All flasks and plates were incubated in a humidified atmosphere of 95% air and 5% CO2 at 37° C.

BxPC-3 pancreatic carcinoma cell line was cultured in RPMI 1640 with 10% FBS.

NMuMG (normal mouse mammary gland cells) cell line was grown in DMEM media with 4.5 g/l glucose, 10 μg/ml insulin and 10% FBS.

All the above cells were seeded at 2,500 to 5,000 cells/well in 96 well plates. The cells were treated with oligonucleotide compounds in fresh media (100 μl total volume) 24 hours after seeding. The media was replaced with fresh media without oligonucleotides 24 hours after treatment and every 48 hours for 6 to 7 days or until the control cells were 80 to 100% confluent. The inhibitory effect of oligonucleotide was evaluated using an MTT staining technique.

Cell Growth Inhibition Assay (FIGS. 26, 50-53; Descriptions Referenced in U.S. Pat. No. 7,524,827)

Cell growth inhibition was assessed using 3-[4,5-Dimethyl-thiazol-2-yl]-2,5diphenyltetrazolium bromide (MTT) purchased from Sigma Chemical (St. Louis, Mo.). Cells were resuspended in culture media at 50,000 cells/ml and 100 μl was distributed into each well of a 96-well, flat bottomed plate (Costar Corning, N.Y., USA) and incubated for 24 hours. Media was changed to 100 μl fresh media containing the desired concentration of oligonucleotides and incubated for 24 hours. Controls had media with pure sterile water equal to the volume of oligonucleotide solution. The media was changed without further addition of oligonucleotides every 24 hours until the control cultures were confluent (6 to 7 days). Thereafter the media was removed and plates were washed two times with phosphate-buffered saline (PBS) and 100 μl of serum free media containing 0.5 mg/ml MTT dye was added into each well and incubated for 1 hour at 37° C. The media with dye was removed, washed with PBS and 100 μl of dimethyl sulfoxide (DMSO) was added to solubilize the reactive dye. The absorbance values were read using an automatic multiwell spectrophotometer (Bio-Tek Microplate Autoreader, Winooski, Vt., USA). Each treatment was repeated at least 3 times with 8 independent wells each time. Included in Tables 4 and 5 are the sequences for the control and negative control oligonucleotides used in the experiments.

C. Preparation and Formulation of Oligonucleotides

Any of the known methods of oligonucleotide synthesis can be used to prepare the modified oligonucleotides of the present invention. In some embodiments utilizing methylated oligonucleotides the nucleotide, dC is replaced by 5-methyl-dC where appropriate, as taught by the present invention. The modified or unmodified oligonucleotides of the present invention are most conveniently prepared by using any of the commercially available automated nucleic acid synthesizers. They can also be obtained from commercial sources that synthesize custom oligonucleotides pursuant to customer specifications.

While oligonucleotides are a preferred form of compound, the present invention comprehends other oligomeric oligonucleotide compounds, including but not limited to oligonucleotide mimetics such as are described below. The oligonucleotide compounds in accordance with this invention preferably comprise from about 20 to about 34 nucleobases (i.e., from about 20 to about 34 linked bases), although both longer and shorter sequences may find use with the present invention.

Specific examples of preferred compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

In some embodiments, oligonucleotides of the invention are oligonucleotides with phosphorothioate backbones and oligonucleosides with heteroatom backbones, and in particular —CH2, —NH—O—CH2-, —CH2-N(CH3)-O—CH2- [known as a methylene (methylimino) or MMI backbone], —CH2-O—N(CH3)-CH2-, —CH2-N(CH3)-N(CH3)-CH2-, and —O—N(CH3)-CH2-CH2- [wherein the native phosphodiester backbone is represented as —O—P—O—CH2-] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)nO]mCH3, O(CH2)nOCH3, O(CH2)nNH2, O(CH2)nCH3, O(CH2)nONH2, and O(CH2)nON[(CH2)nCH3)]2, where n and m are from 1 to about 10. Other preferred oligonucleotides comprise one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2CH2OCH3, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH2)2ON(CH3)2 group), also known as 2′-DMAOE, and 2′-dimethylaminoethoxyethoxy (also known in the art as 2′-O-dimethylaminoethoxyethyl or 2′-DMAEOE), i.e., 2′-O—CH2-O—CH2-N(CH2)2.

Other preferred modifications include 2′-methoxy(2′-O—CH3), 2′-aminopropoxy(2′-OCH2CH2CH2NH2) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes pharmaceutical compositions and formulations that include the antisense compounds of the present invention as described below.

D. Cocktails

In some embodiments, the present invention provides cocktails comprising two or more oligonucleotides directed towards promoter regions of genes (e.g., oncogenes). In some embodiments, the two oligonucleotides hybridize to different regions of the promoter of the same gene. In other embodiments, the two or more oligonucleotides hybridize to promoters of two different genes. The present invention is not limited to a particular mechanism. Indeed, an understanding of the mechanism is not necessary to practice the present invention. Nonetheless, it is contemplated that the combination of two or more compounds of the present invention provides an inhibition of cancer cell growth that is greater than the additive inhibition of each of the compounds administered separately.

### V. Research Uses

The present invention is not limited to therapeutic applications. For example, in some embodiments, the present invention provides compositions and methods for the use of oligonucleotides as a research tool.

A. Kits

For example, in some embodiments, the present invention provides kits comprising oligonucleotides specific for inhibition of a gene of interest, and optionally cell lines (e.g., cancer cells lines) known to express the gene. Such kits find use, for example, in the identification of metabolic pathways or the involvement of genes in disease (e.g., cancer), as well as in diagnostic applications. In some embodiments, the kits further comprise buffer and other necessary reagents, as well as instructions for using the kits.

B. Target Validation

In some embodiments, the present invention provides methods and compositions for use in the validation of gene targets (e.g., genes suspected of being involved in disease). For example, in some embodiments, the expression of genes identified in broad screening applications (e.g., gene expression arrays) as being involved in disease is downregulated using the methods and compositions of the present invention. The methods and compositions of the present invention are suitable for use in vitro and in vivo (e.g., in a non-human animal) for the purpose of target validation. In other embodiments, the compounds of the present invention find use in transplantation research (e.g., HLA inhibition).

C. Drug Screening

In other embodiments, the methods and compositions of the present invention are used in drug screening applications. For example, in some embodiments, oligonucleotides of the present invention are administered to a cell (e.g., in culture or in a non-human animal) in order to inhibit the expression of a gene of interest. In some embodiments, the inhibition of the gene of interest mimics a physiological or disease condition. In other embodiments, an oncogene or disease causing gene is inhibited. Test compounds (e.g., small molecule drugs or oligonucleotide mimetics) are then administered to the test cell and the effect of the test compounds is assayed.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

### VI. Compositions and Delivery

In some embodiments, the oligonucleotide compounds of the present invention are formulated as pharmaceutical compositions for delivery to a subject as a pharmaceutical. The novel antigen compounds of the present invention find use in the treatment of a variety of disease states and conditions in which it is desirable to inhibit the expression of a gene or the growth of a cell. In some preferred embodiments, the compounds are used to treat disease states resulting from uncontrolled cell growth, for example including, but not limited to, cancer. The present invention is not limited to the treatment of a particular cancer. The oligonucleotide compounds of the present invention are suitable for the treatment of a variety of cancers including, but not limited to, breast, colon, lung, stomach, pancreatic, bladder, leukemia, and lymphoma. In other preferred embodiments, the compounds are used to treat disease states resulting from gene expression, for example including, but not limited to, non cancer diseases. The below discussion provides exemplary, non-limiting examples of formulations and dosages.

A. Pharmaceutical Compositions

The present invention further provides pharmaceutical compositions (e.g., comprising the oligonucleotide compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer); intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, needless injectors, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, nanoparticle, nanocrystal, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), cochleates (Patent application numbers 20080242625 and 20120294901) also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles or nanocrystals. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, phosphatidylserine, calcium, and polyethyleneglycol (PEG).

Certain embodiments of the invention provide pharmaceutical compositions containing (a) one or more oligonucleotide compounds and (b) one or more other chemotherapeutic agents that function by a non-oligonucleotide mechanism. Examples of such chemotherapeutic agents include, but are not limited to, cytotoxic agents, small molecule protein inhibitors, antibodies, and anti-sense anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin, lenalomide, and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-oligonucleotide chemotherapeutic agents are also within the scope of this invention. Two or more combined compounds may be used together or sequentially.

B. Delivery

The oligonucleotide compounds of the present invention may be delivered using any suitable method. In some embodiments, naked DNA is administered. In other embodiments, lipofection is utilized for the delivery of nucleic acids to a subject. In still further embodiments, oligonucleotides are modified with phosphothioates for delivery (See e.g., U.S. Pat. No. 6,169,177, herein incorporated by reference).

In some embodiments, nucleic acids for delivery are compacted to aid in their uptake (See e.g., U.S. Pat. Nos. 6,008,366, 6,383,811 herein incorporated by reference). In some embodiment, compacted nucleic acids are targeted to a particular cell type (e.g., cancer cell) via a target cell binding moiety (See e.g., U.S. Pat. Nos. 5,844,107, 6,077,835, each of which is herein incorporated by reference).

In some embodiments, oligonucleotides are conjugated to other compounds to aid in their delivery. For example, in some embodiments, nucleic acids are conjugated to polyethylene glycol to aid in delivery (See e.g., U.S. Pat. Nos. 6,177,274, 6,287,591, 6,447,752, 6,447,753, and 6,440,743, each of which is herein incorporated by reference). In yet other embodiments, oligonucleotides are conjugated to protected graft copolymers, which are chargeable” drug nano-carriers (PharmaIn). In still further embodiments, the transport of oligonucleotides into cells is facilitated by conjugation to vitamins (Endocyte, Inc, West Lafayette, Ind.; See e.g., U.S. Pat. Nos. 5,108,921, 5,416,016, 5,635,382, 6,291,673 and WO 02/085908; each of which is herein incorporated by reference). In other embodiments, oligonucleotides are conjugated to nanoparticles (e.g., NanoMed Pharmaceuticals; Kalamazoo, Mich.).

In preferred embodiments, oligonucleotides are enclosed in lipids (e.g., liposomes or micelles) to aid in delivery (See e.g., U.S. Pat. Nos. 6,458,382, 6,429,200; each of which is herein incorporated by reference). Preferred liposomes include, but are not limited to amphoteric liposomes (e.g., SMARTICLES,). In still further embodiments, oligonucleotides are complexed with additional polymers to aid in delivery (See e.g., U.S. Pat. Nos. 6,379,966, 6,339,067, 5,744,335; each of which is herein incorporated by reference and Intradigm Corp., Rockville, Md.). Cochleates see e.g. Patent application number: 20080242625 and 20120294901.

In still further embodiments, the controlled high pressure delivery system developed by Mirus (Madison, Wis.) is utilized for delivery of oligonucleotides.

C. Dosages

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. In some embodiments, the oligonucleotide is introduced to the host animal at a dosage of between 0.1 mg to 10 g, and preferably at a dosage of between 00.1 mg to 100 mg per kg of body weight or 1 to 300 mg per meter squared body surface area. The administering physician can determine optimum dosages, dosing methodologies and repetition rates. Optimum dosages may vary depending on the relative potency of individual oligonucleotides, and the delivery means, and can generally be estimated based on EC50s found to be effective in in vitro and in vivo animal models or based on the examples described herein. In general, dosage is from 10 mg to 10 g per kg of body weight, and may be given once or more daily, weekly, monthly or yearly. In some embodiments, dosage is continuous (e.g., intravenously) for a period of from several minutes to several days or weeks. In some embodiments, treatment is given for a defined period followed by a treatment free period. In some embodiments, the pattern of continuous dosing followed by a treatment free period is repeated several times (e.g., until the disease state is diminished).

The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 10 mg to 10 g, preferably from 1 mg to 5 mg, and even more preferably from 0.1 mg to 30 mg per kg of body weight or 0.1 mg/m² to 200 mg/m², once or more daily, to once every 20 years.

### VII. Customized Patient Care

In some embodiments, the present invention provides customized patient care.

The compositions of the present invention are targeted to specific genes unique to a patient's disease (e.g., cancer). For example, in some embodiments, a sample of the patient's cancer or other affected tissue (e.g., a biopsy) is first obtained. The biopsy is analyzed for the presence of expression of a particular gene (e.g., oncogene). In some preferred embodiments, the level of expression of an gene in a patient is analyzed. Expression may be detected by monitoring for the presence of RNA or DNA corresponding to a particular oncogene. Any suitable detection method may be utilized, including, but not limited to, those disclosed below. 5 10 15 20

Following the characterization of the gene expression pattern of a patient's gene of interest, a customized therapy is generated for each patient. In preferred embodiments, oligonucleotide compounds specific for genes that are aberrantly expressed in the patient (e.g., in a tumor) are combined in a treatment cocktail. In some embodiments, the treatment cocktail further includes additional chemotherapeutic agents (e.g., those described above). The cocktail is then administered to the patient as described above.

In some embodiments, the analysis of cancer samples and the selection of oligonucleotides for a treatment compound is automated. For example, in some embodiments, a software program that analyses the expression levels of a series of oncogenes to arrive at the optimum selection and concentration of oligonucleotides is utilized. In some embodiments, the analysis is performed by the clinical laboratory analyzing the patient sample and is transmitted to a second provider for formulation of the treatment cocktail. In some embodiments, the information is transmitted over the Internet, thus allowing for the shortest possible time in between diagnosis and the beginning of treatment.

A. Detection of RNA

In some embodiments, detection of oncogenes (e.g., including but not limited to, those disclosed herein) is detected by measuring the expression of corresponding mRNA in a tissue sample (e.g., cancer tissue or other biopsy). In other embodiments, expression of mRNA is measured in bodily fluids, including, but not limited to, blood, plasma, lymph, serum, mucus, and urine. In some preferred embodiments, the level of mRNA expression in measured quantitatively. RNA expression may be measured by any suitable method, including but not limited to, those disclosed below.

In some embodiments, RNA is detected by Northern blot analysis. Northern blot analysis involves the separation of RNA and hybridization of a complementary labeled probe. In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In yet other embodiments, mRNA or transcript numbers are measured using branched DNA technology (e.g. QuantiGene). Branched DNA (bDNA) quantitatively measures gene expression by a sandwich nucleic acid hybridization method that uses bDNA probes specific to the target RNA. The signal from captured target RNA is amplified and enhances assay sensitivity thereby eliminating the need to amplify target RNA by traditional PCR-based gene expression techniques. Furthermore, bDNA assays measure RNA directly from the sample source, without RNA purification or enzymatic manipulation, potentially avoiding inefficiencies and variability introduced by errors inherent to these processes.

B. Detection of Protein

In other embodiments, gene expression of oncogenes is detected by measuring the expression of the corresponding protein or polypeptide. In some embodiments, protein expression is detected in a tissue sample. In other embodiments, protein expression is detected in bodily fluids. In some embodiments, the level of protein expression is quantitated. Protein expression may be detected by any suitable method. In some embodiments, proteins are detected by their binding to an antibody raised against the protein. The generation of antibodies is well known to those skilled in the art.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates an expression profile based on the presence or absence of a series of proteins corresponding to oncogenes is utilized.

In other embodiments, the immunoassay described in U.S. Pat. Nos. 5,599,677 and 5,672,480; each of which is herein incorporated by reference.

### VIII Listing of DNAi Sequences

The following sequences in Table 3 are provided as additional non-limiting examples of preferred embodiments of the invention.

TABLE 3
New DNAi Sequences

| | | | | Location |
|---|---|---|---|---|
| | | | | relative to 5′ |
| | | | | upstream |
| | | | | region from |
| | Design | Sequence | | gene start |
| Target | ID | ID No: | Sequence (5′-3′) | site |
| Survivin | SU1 | 1 | GAGCGCACGCCCTCTTAGGCGG | 73 |
| Survivin | SU2 | 75 | CACCCCGAGGTACGATCAGTGCGTACC | 2990 |
| Survivin | SU1_02 | 155 | GAGCGCACGCCCTCTTAGGCG | 73 |
| Survivin | SU1_03 | 229 | GAGCGCACGCCCTCTTAGGCGGTCCA | 73 |
| Survivin | | 303 | GTCGCCCCTGGGTCCTGCTGATTGGC | 1918 |
| Survivin | | 322 | CAGCGAGCCTGGGCCCCATCGGCACATCT | 2905 |
| Survivin | | 357 | CCCGCGGCCTTCTGGGAGTAGAGGC | 102 |
| Survivin | | 431 | TCCCGGCGAGTACATCGTTGACTGCACG | 675 |
| Survivin | | 481 | AACCTCCTCCCCGCCACGGGTT | 1229 |
| Beclin-1 | BE1 | 515 | CGACGCCCTTGACCTCCGGCCCGGGGT | 39 |
| Beclin-1 | BE2 | 550 | CTGCGCCGTTCCCTCTAGGAATGG | 111 |
| Beclin-1 | | 572 | GAAGCGACGCCCTTGACCTCCGGCCCGG | 35 |
| Beclin-1 | | 607 | CCCCCGATGCTCTTCACCTCGGG | 261 |
| Beclin-1 | | 712 | CGGGTCGGCCCCGGAGCGAGGCC | 335 |
| Beclin-1 | | 817 | GCCCGGCAGCGGCCCCCAGAGGCCG | 475 |
| Beclin-1 | | 847 | CGGTCTACCGCGGAGGCACTGTGGCCTCGG | 308 |
| Beclin-1 | | 952 | ACAAAAACTAGCCGGGCGTGGTGGGGCACGCC | 735 |
| STAT3 | ST1 | 984 | GGCCGAGGCACGCCGTCATGCA | −18 |
| STAT3 | ST2 | 985 | CCGGCCCTTGGCACCACGTGGTGGCGA | 345 |
| STAT3 | | 986 | TTGTTCCCTCGGCTGCGACGTCG | −135 |
| STAT3 | | 987 | CAGTCTGCGCCGCCGCAGCTCCGG | −92 |
| STAT3 | | 988 | CAGTGCGTGTGCGGTACAGCCG | 45 |
| STAT3 | | 989 | TGTGCTGGCTGTTCCGACAGTTCGGT | 140 |
| STAT3 | | 990 | TAACTACGCTATCCCGTGCGGCC | 1998449 |
| STAT3 | | 991 | TCGCCCAGCCCCAGCCTGGCCGAGGC | −35 |
| HIF1A | HI1 | 992 | CAGGCCGGCGCGCGCTCCCGCAA | 390 |
| HIF1A | HI2 | 1048 | GGACGGGCTGCGACGCTCACGTGC | 539 |
| HIF1A | | 1090 | GAGGTGGGGGTGCGAGGCGGGAAACCCCTCG | 108 |
| HIF1A | | 1129 | CAATCGCCGGGGTCCGGGCCCGGC | 162 |
| HIF1A | | 1130 | TGGCCGAAGCGACGAAGAGGG | 232 |
| HIF1A | | 1142 | GGGCGGAGGCGCGCTCGGGCGCG | 325 |
| HIF1A | | 1214 | CACGGCGGGCGGCCCCCAGGCTCGC | 26 |
| HIF1A | | 1270 | CAGGCCGGCGCGCGCTCCCGCAAGCCCG | 390 |
| HIF1A | | 13680 | CGATTGCCGCCCAACTCTGCTGGG | 789 |
| IL-8 | IL8-1 | 1314 | ACGTCCCATTCGGCTCCTGAGCCA | 2868 |
| IL-8 | IL8-3 | 1331 | GACGTTGACGAAGTCTATCACCCAA | 2939 |
| IL-8 | | 1341 | ACGGAGTATGACGAAAGTTTTC | 257 |
| IL-8 | | 1342 | GAGCGAGACTCCCGTCTAAA | 3259 |
| KRAS | | 1535 | GCCGGGCCGGCTGGAGAGCGGGTC | 5803 |
| KRAS | | 1538 | TCGCCCCTCCTCCGAGACTTTC | 6626 |
| KRAS | | 1584 | GCACCCCGCCACCCTCAGGGTCGGC | 6029 |
| KRAS | | 1633 | GAGCCGCCGCCACCTTCGCCGCCGC | 5475 |
| KRAS | | 1697 | CGGCATAGTTCCCCGCCTTAC | 2002 |
| KRAS | KR16 | 1730 | CGGCCCGAGCCTCCGTGACGAGTGC | 146348 |
| KRAS | KR17 | 1767 | CTGGGAGGGGATCCCTCACCGAGAG | 3328 |
| MTTP | | 1784 | AACCGCCGTAGCCTCCACTGCG | 28 |
| MTTP | | 1870 | TGGCCGCAGTTCGATGACGTAAGACG | 1 |
| ApoC-III | | 1956 | GAGTCGGTGGTCCAGGAGGGGCCGC | 939 |
| ApoC-III | | 1957 | CTGCGGCTGAGGTGTCATTCGTGACTCAG | 3539 |
| ApoC-III | | 1992 | GCGGGCGGGTGAGACAGAAGCGCC | 3455 |
| ApoC-III | | 1993 | CCTCGCGAGCGTGGGTGCACGC | 3310 |
| ApoC-III | | 2028 | CGATGTCTCCCTCGAGATCACA | 3042 |
| ApoC-III | | 2054 | GGACGGACGGATATCTGAGGCCAG | 1520 |
| ApoC-III | | 2062 | CGTCCCCGCCACGTTGAAAGGC | 3279 |
| ApoC-III | | 2089 | TCTCGGACATGCTCAAATGGTGCAGGCG | 3405 |
| ApoC-III | | 2108 | CACCGACAGGAGCCAATAGTGCAACG | 4201 |
| ApoC-III | | 2127 | GTCCGGCAGAGGGACCCATGCTGACG | 4265 |
| ApoC-III | | 2136 | CGTGAGGCACATGTCCGTGTG | 2836 |
| ApoC-III | | 2170 | CAGATGCAGCAAGCGGGCGGGAGAG | 123 |
| ApoC-III | | 2176 | CCACGCTGCTGTCCCGCCAGCCCTGCAG | 173 |
| ApoC-III | | 2206 | ACCCGCCCCCACCCTGTGTGCCCCC | 601 |
| ApoC-III | | 2225 | CGCTCAGAGCCCGAGGCCTTTG | 677 |
| ApoB | | 2252 | CGGTGGGGCGGCTCCTGGGCTGC | 10 |
| ApoB | | 2329 | CCTCGCGGCCCTGGCTGGCTGGGCG | 46 |
| ApoB | | 2406 | AACCGAGAAGGGCACTCAGCCCCG | 88 |
| ApoB | | 2440 | CGGCGCCCGCACCCCATTTATAGG | 136 |
| ApoB | | 2451 | GTCCAAAGGGCGCCTCCCGGGCC | 195 |
| ApoB | | 2475 | CGTCTTCAGTGCTCTGGCGCGGCC | 341 |
| ApoB | | 2513 | CACCGGAAGCTTCAGCCAGCGCTCGCTG | 988 |
| ApoB | | 2552 | CGAGTGGGAGGCGGCCAGGAGCAAGCCG | 1281 |
| ApoB | | 2553 | CGTACACTCACGGAAATGCTGTAAAG | 2533 |
| ApoB | | 2576 | CGTCACAGCCAATAATGAGCGTACGC | 4862 |
| IL17 | | 2601 | CTTGTTTGTATCCGCATGGCTGTGCTC | 4451 |
| IL17 | | 2616 | CGAGACCGTTGAGGTGGAGTG | 3148 |
| IL17 | | 2635 | GGTCACTTACGTGGCGTGTCGC | 107 |
| IL17 | | 2664 | GACAAAATGTAGCGCTATCG | 55 |
| MMP2 | | 2666 | GCTCCCTGGCCCCGCGCGTCGC | 9 |
| MMP2 | | 2732 | CCGCGGCGCAGGGCTGCGCTCCGAG | 85 |
| MMP2 | | 2865 | GCCGCCTGCTACTCCTGGCCTC | 453 |
| MMP2 | | 2869 | GCGCACTCGGGCCCGCCCCTCTCTGCCC | 361 |
| MMP2 | | 2891 | CGCTCCGAGGGTCCGCTGGCTCGG | 101 |
| MMP2 | | 3024 | GTCCACCCTCAGTGCACGACCTCGT | 478 |
| MMP2 | | 3066 | CACCGCCTGAGGAAGTCTGGATGC | 239 |
| MMP2 | | 3101 | TGCCTCTCTCGCGATCTGGGCG | 512 |
| MMP2 | | 3131 | GAGGGACGCCGGCTTGGCTAGGAC | 618 |
| FAP | | 3154 | CAGAGCGTGGGTCACTGGATCT | 39 |
| FAP | | 3171 | CACCAACATCTGCTTACGTTGAC | 272 |
| FAP | | 3177 | TCCACGGACTTTTGAATACCGTGC | 133 |
| P-selectin | | 3184 | TAGCTACGAATAAAGAAATTTGTAG | 2694 |
| IL6 | | 3185 | CACCGCGTGGCTTCTGCCACTTTC | 723 |
| IL6 | | 3206 | TACGGACGCAGGCACGGCTCTAG | 1117 |
| IL6 | | 3226 | CAGCTCCGCAGCCGTGCACTGTG | 1722 |
| IL6 | | 3255 | CTTCACCGATTGTCTAAACAGAGAC | 1525 |
| IL6 | IL6_1 | 3256 | TTCGTTCCCGGTGGGCTCGAGGGC | 35 |
| IL6 | | 3276 | TGCTTCCGCGTCGGCACCCAAG | 1150 |
| IL23 | | 3300 | TCCCTGCATTGTAAGGCCCGCC | 195 |
| IL23 | | 3319 | CACAGCGGGGATGGGGTGGGAGGG | 414 |
| IL23 | | 3320 | GACGTCAGAATGAGGCCATCG | 1296 |
| IL23 | | 3341 | GAGCCAGCACGGTGGTGGGCGCC | 1651 |
| IL23 | | 3365 | GCGTTTGTCCCACCGGCGCCCCG | 4861 |
| IL23 | | 3479 | TAACGCCACCCAACAAGTCCGGCG | 4830 |
| AKT1 | | 3593 | GAGGCTCCCGCGACGCTCACGCG | 8 |
| AKT1 | | 3646 | TACCGGGCGTCTCAGGTTTTGCC | 843 |
| AKT1 | | 3669 | TCCGAGCCGCGCACGCCTCAGGC | 1562 |
| AKT1 | | 3703 | CACCAACGGACTCCGTCCGCCC | 2010 |
| AKT1 | | 3770 | CCGCCGGCTGCCTCGCTGGCCCAGCG | 2464 |
| AKT1 | | 3927 | TCTCGGGTCCCGGCCTCGCCCGGCGGAGC | 2556 |
| AKT1 | | 4084 | CATTCTGGCGGCGCCGCGGCTCGCG | 2730 |
| AKT1 | | 4228 | CACCGGGCCGCCGCGTCCGGGCGCG | 2838 |
| AKT1 | AKT4 | 4338 | CACATCCGCCTCCGCCGCCCGG | 3160 |
| CRAF | | 4339 | GCGCGAGCCCTACTGGCAGTCG | 390 |
| CRAF | | 4462 | CGGGGCGTGGCCTAGCGATCTGGTGGCCG | 467 |
| CRAF | | 4517 | TTTCGAAGCTGAAGAGGTTAGGCGACG | 499 |
| CRAF | | 4519 | CGACGCTGACTTGCTTTCAGGAG | 521 |
| CRAF | | 4533 | AATCGAGAAGAACCGGCTTTCGG | 555 |
| CRAF | | 4556 | CTTTGACGCGTCCTCTCCGGGC | 689 |
| CRAF | | 4585 | CGGCTCCGCCACTTGACAGCTATGTGG | 728 |
| CRAF | | 4605 | AGGCGGAGATTGCGGTGAGCCGAAATCGCG | 1582 |
| CRAF | | 4609 | AGGCCGCCCCAACGTCCTGTCGTTCGGCGG | 12 |
| CRAF | | 4677 | TCTCGCCCGCTCCTCCTCCCCGCGGCGGGTG | 47 |
| CRAF | | 4745 | CGGGAGGCGGTCACATTCGGCGCG | 84 |
| CRAF | | 4782 | CGGAGCCCCGAGCAGCCCCCGCATCG | 124 |
| CRAF | | 4871 | CGCGCTCCGCGCCTCAGGGCACGCGCC | 157 |
| CRAF | | 4960 | AGCCGTTCCCGCCTCACAATCG | 234 |
| CRAF | | 4984 | CCGCCATCTAAGATGGCGGCC | 270 |
| CRAF | | 5047 | CGGGCGGCCCAGACGAGCGAGCCCTCG | 314 |
| CRAF | | 5110 | CGTCCTCCCGACCTGCGACGCCACCGGC | 351 |
| Beta- | | 5233 | CGCATATTACTGGGTAAACTCTGTG | 1411 |
| catenin | | | | |
| Beta- | | 5234 | CACGCTGGATTTTCAAAACAGTTG | 5 |
| catenin | | | | |
| PCSK9 | | 5235 | CAGGGCGCGTGAAGGGGCGCGCGG | 120 |
| PCSK9 | | 5236 | GACGCGTCCCGGCCCGCCCGAGC | 179 |
| PCSK9 | | 5285 | GACGCCTGGGGCGCGCAGATCAC | 341 |
| PCSK9 | | 5341 | CAGGCCGGCGCCCTAGGGGCTCC | 494 |
| PCSK9 | | 5359 | CACGCCGGCGGCGCCTTGAGCC | 56 |
| PCSK9 | | 5402 | CAGGTTTCGGCCTCGCCCTCCC | 408 |
| PCSK9 | | 5445 | CATCGAGCCCGCCATCGCAGCAC | 1307 |
| PCSK9 | | 5473 | GAGCGCCTCGACGTCGCTGCGGAAACC | 273 |
| MEK1 | | 5534 | CAAGTCCGGGCCGCGGGCCCCGGGGC | 93 |
| MEK1 | MEK1_2 | 5716 | GCGCCCCGCGCGGTCCCGTCAGCGC | 133 |
| MEK1 | | 5898 | GCGGAGCGGGCTGAACGTGCG | 249 |
| MEK1 | | 5900 | GACTGGAGGCCGGGGGAGGGGCGGGG | 433 |
| MEK1 | | 5901 | GACCCGGGTAACGCGCTTCCAAC | 5 |
| MEK1 | MEK1_1 | 5924 | CACTCGGCTCCGCCCCTATTGC | 507 |
| MEK1 | | 6000 | TACGTCACGGGAGCGCGGCGCAC | 578 |
| MEK1 | | 6077 | GTCGCGGACGCCGTGGCGCCCTCTGTC | 619 |
| MEK1 | | 6154 | CACTCGCCGTCATGCCCGGATCC | 1183 |
| MEK2 | | 6182 | CGCCGCAGCCCGAGTCCGAGAGG | 226 |
| MEK2 | | 6202 | GAGGGGCGCTGGGGCTGAGGCGAGCG | 165 |
| MEK2 | | 6203 | CTCGCGATAACGGGATCGGGAGCCGCG | 290 |
| MEK2 | MEK2_1 | 6235 | CCGACGCGAGGCGGTGCCGGGACCGG | 391 |
| MEK2 | | 6240 | CACGGCGCGTGTGCCCAAGCGC | 436 |
| MEK2 | | 6299 | CGTGGACACACGCCCCTAGCCC | 643 |
| MEK2 | | 6341 | TAGACACTTCGGTGAATCGTGCCGC | 1622 |
| CD4 | | 6373 | GAGCCACTGCGCCCGGCCTCATTAAGGGCAT | 1818 |
| CD4 | | 6406 | CGAACAACTTCATTACAATTCGACAAGCGC | 2632 |
| CD4 | | 6407 | CGTAGTTAAGCGTGTACCAGCCCAAGGC | 2522 |
| CD4 | | 6421 | GAGCGGTGACCGTGTCTGTCTTAG | 3084 |
| CD4 | | 6447 | CGGTTTGCAGATTCCAGACCCGATGGACG | 4433 |
| WNT1 | | 6466 | CGCGCGCCCGCCTCACTCAGCTGAGCG | 442 |
| WNT1 | | 6537 | CGTCATTCTGTTGCCCTTTGTACCTCG | 1226 |
| WNT1 | | 6545 | CGCCACGGGCGCATCCATCCCTCCTGGG | 4454 |
| WNT1 | | 6579 | CACCGCCCTCTAGCCGCCTGCGGG | 4960 |
| WNT1 | | 6580 | TTGCGGCGACTTTGGTTGTTGCCCGCGACGGT | 34 |
| Clusterin | | 6636 | CGTCCCGCCCACCTGCTGCCTGCAGCAG | 78 |
| Clusterin | | 6660 | CGACAATCAGCGAGGCACACAGGCT | 330 |
| Clusterin | | 6689 | CGGAGAGTAGAGAGGGTTCGCAGTGGCCC | 718 |
| Clusterin | | 6690 | CCACGGGGCACAGGCCATAGCCCCG | 890 |
| Clusterin | | 6709 | CTCGTGCTCTCAGGCGGCGGTTGCGCCG | 3865 |
| Clusterin | | 6752 | CCGGGAGGTGGGGGCCGGTGCAGCACCGG | 4260 |
| Clusterin | | 6753 | TCGCGTGCCCATCTGGGAGCCCCTCTCACG | 4395 |
| NRAS | | 6774 | CCCCGCCCTCAGCCTAAGCAATGGA | 234 |
| NRAS | | 6793 | GACCCCGGAACCGCCATGAACAGCCC | 559 |
| NRAS | | 6818 | CCCGCTACGTAATCAGTCGGCGCCCCA | 613 |
| NRAS | | 6961 | AACGCAAAAACACCGGATTAATATCGGCCT | 142 |
| NRAS | | 6963 | ATAAACGGCCTCTTTACCCAGAGATCA | 850 |
| NRAS | | 6971 | CGCCACCTTAAGTTTTTCCAGGCTGC | 1779 |
| EZH2 | EZH2_2 | 6986 | TCCCGACAAGGGGTGACAGAGGC | 1002 |
| EZH2 | | 7002 | CGTGAATTCAAGAGTTGCTTAGGCC | 1059 |
| EZH2 | | 7003 | GACTACCGGTGCCCGCCACCACGCCAGGC | 2856 |
| EZH2 | | 7035 | GACCGCCCCCCGCCAACCCCACAGCGG | 3459 |
| HDAC1 | | 7075 | CGCCTCCCGTCCCTACCGTCAGTCGGT | 7 |
| HDAC1 | | 7141 | CGGTCCGTCCGCCCTCCCGCCCGCGG | 30 |
| HDAC1 | | 7207 | CGCCAACTTGTGGTCCTACAGTCAACAAG | 1740 |
| HDAC1 | | 7226 | CGCAGACACGGGCCCGGAACTCGG | 173 |
| HDAC1 | | 7258 | CGCCCGGCCTAGGAGGGCAGGTTTCTC | 1252 |
| PD-1 | | 7297 | TGCCGCCTTCTCCACTGCTCAGGCG | 23 |
| PD-1 | | 7316 | ACCGCCTGACAGCTGGCGCGGCTGCCTGGC | 1061 |
| PD-1 | PD1 | 7379 | CTGCGAGGCGCGGCCACGGCG | 1171 |
| PD-1 | | 7396 | CGAGGAGGAAAGGCAGGCGGAGTCCG | 3395 |
| PD-1 | | 7397 | CAGCGAAGCTGCAGAACGTCCCCATCACCACG | 4268 |
| PD-1 | | 7439 | CGACAGCCGTGGGAAGGTGCAGTACG | 4388 |
| PD-1 | | 7440 | CGGGATTCCCTGGAGATGCCTCCAGCGCG | 4422 |
| PD-1 | | 7466 | AGGCGGTCCCAGGGCTCAGGTGTGGG | 2229 |
| PD-1 | | 7498 | GCGTGCACCCCGTGGCCAGCTC | 3813 |
| PD-1 | | 7526 | CAACGTACACGCAATCCACAAC | 2832 |
| TNFa | | 10095 | CGGGGAAAGAATCATTCAACCAGCGG | 254 |
| TNFa | TNF1 | 10096 | CGGTTTCTTCTCCATCGCGGGGGCG | 350 |
| TNFa | | 10129 | CTGCTCCGATTCCGAGGGGGGTCTTCT | 438 |
| TNFa | | 10154 | CTCCGTGTGGGGCTCTGGTCGGCAGCT | 1490 |
| TNFa | | 10207 | CGCAGCCCCGTGGTACATCGAGTGCAGC | 2178 |
| MIF1 | | 12470 | GACCCGCGCAGAGGCACAGACGC | 42 |
| MIF1 | | 12490 | CGCCACCGCCGGCGCCAGGCCCCGCCCCCGCG | 143 |
| MIF1 | | 12701 | CGTTCCTCCAGCAACCGCCGCTAAGCCCGGCG | 258 |
| MIF1 | | 12912 | CGCCTGCCTCGGCTCGACCCCCGCAG | 202 |
| MIF1 | | 13123 | CGGCTAGAAATCGGCCTGTTCCGGCCTCGCCT | 317 |
| MIF1 | | 13174 | CGGGGGTGGGGATGCGGCGGTGAACCCG | 404 |
| MIF1 | | 13175 | CGCGGCAGGTGAGAGGGGAGCTGCCCCTGCG | 588 |
| MIF1 | | 13176 | CGCGTGCACGTGTGTCCACATGAGTGC | 3676 |
| MIF1 | MIF1_1 | 13203 | CGCCACCGCCGGCGCCAGGCCCCGCC | 137 |
| MIF1 | MIF1_2 | 13414 | CGCGGCAGGTGAGAGGGGAGCTGCCC | 583 |
| TTR | | 11359 | CAACGCCCTGGCTCGAGTGCAGTGGCACG | 803 |
| TTR | | 11432 | CTACTATCTCAGATACTCGGCCAACTCG | 1776 |
| TTR | | 11450 | CACGCGTTTCAGCACTGCACCCTGTTG | 2112 |
| HBV | | 9179 | CCGATTGGTGGAGGCAGGAGGAGG | 72 |
| HBV | | 9180 | CGAGATTGAGATCTTCTGCGACGCGG | 780 |
| HBV | | 9235 | CGCGGCGATTGAGACCTTCGTC | 801 |
| HBV | | 9290 | CGTCTGCGAGGCGAGGGAGTTCTTCT | 819 |
| HBV | | 9345 | CGATACAGAGCAGAGGCGGTGT | 1200 |
| HBV | | 9346 | CGCGTAAAGAGAGGTGCGCCCCGTGG | 1674 |
| HBV | | 9360 | ACGGGTCGTCCGCGGGATTCAGCGCCG | 1754 |
| HBV | | 9409 | CGTCCCGCGCAGGATCCAGTTGG | 1800 |
| HBV | | 9432 | CGGCTGCGAGCAAAACAAGCTGCTAG | 1909 |
| HBV | | 9468 | CGCATGCGCCGATGGCCTATGGCCAA | 1978 |
| HBV | | 9496 | CGCCGCAGACACATCCAGCGATA | 2826 |
| HBV | | 9525 | GCTCCAGACCGGCTGCGA | 1900 |
| HBV | | 9561 | CGTCCATCGCAGGATCCAGTTGG | 1800 |
| HBV | | 9562 | CGCCGCAGACACATCCAGCGATA | 2826 |
| HBV | | 9591 | CAAATGGCACTAGTAAACTGAG | 2524 |
| HBV | | 9592 | GAGATTGAGATCTGCGGCGACGCGG | 780 |
| HBV | | 9593 | CGACGCGGCGATTGAGATCTTCGTCTG | 801 |
| HBV | | 9594 | AGGGGTCGTCCGCGGGATTCAGCGCCG | 1754 |
| HAMP | | 8999 | CGTGCCGTCTGTCTGGCTGTCCCAC | 1 |
| HAMP | | 9005 | CGAGTGACAGTCGCTTTTATGGGGC | 60 |
| HAMP | | 9035 | CGGGGCATGGCCAGCAGCCGCCAGG | 424 |
| HAMP | | 9086 | CGTGTGCCCGATCCGCACGTGGTGT | 563 |
| HAMP | | 9121 | CGACAGGCTGACGGGCCAAGCTTGG | 2344 |
| HAMP | | 9150 | CGGATGGGCAGGGAGGATACCGTTT | 3109 |
| HAMP | | 9151 | CGTGGGCGGCGGCGGCTGCGTGGTG | 3287 |
| ERBB2 | | 13415 | CGGGAAGAGGATGCGCTGACCTGGC | 2571 |
| ERBB2 | | 13416 | CACGCCCTGGGGAGGAGGCTCGAGAGG | 3267 |
| ERBB2 | | 13437 | CGAGAGGGGCCGAGCCTCTGAAAAA | 3287 |
| ERBB2 | | 13452 | CGTCTGGTCCACAGTCCGATGTCCA | 3944 |
| PARP1 | | 9595 | CCGCCAAAGCTCCGGAAGCCCGACGCC | 14 |
| PARP1 | | 9741 | CCGCCTCGCCGCCTCGCGTGCGCTC | 60 |
| PARP1 | | 9887 | CGGGAACGCCCACGGAACCCGCGTC | 177 |
| PARP1 | | 9933 | CGGGTGGAGCTCTGCGGGCCGCTGC | 269 |
| PARP1 | | 9992 | CGCCGGCCCCAAACTCTTAAGTGTG | 696 |
| PARP1 | | 10014 | CGGGAAGCGCAGGCCCCCGCCTCGG | 749 |
| PARP1 | | 10045 | CGTTCTAACCTGCCGTCCACAGACC | 839 |
| ITGA4 | | 10244 | GCGCTCTCGGTGGGGAACATTCAACAC | 1 |
| ITGA4 | | 10252 | CGGGATGCGACGGTTGGCCAACGG | 54 |
| ITGA4 | | 10278 | CGCAGCGTGTCCGGCGCCAGCGGGC | 102 |
| ITGA4 | | 10299 | CGGCCCACCGCGGGCGGAGCGTTCG | 160 |
| ITGA4 | | 10449 | CGCGCACTCGCCCGGCCCCACTCCCG | 201 |
| ITGA4 | | 10599 | CGCCAGCCGGGAGCTTCGGGTGCTCGCG | 235 |
| ITGA4 | | 10749 | CGGGTACGGGCCGCTGGGTGGGGTCCCG | 272 |
| ITGA4 | | 10899 | GTGCGGAGGCGCAGGGCCGGGCTCCG | 306 |
| ITGA4 | | 10900 | CTACGCGCGGCTGCAGGGGGCGC | 339 |
| ITGA4 | | 10938 | CTGCGCAGGACTCGCGTCCTGGCCCG | 375 |
| ITGA4 | | 11009 | CCCGCAGAGCGCGGGATGGCTC | 411 |
| ITGA4 | | 11080 | CGGACCTGATGGGGCACGGGCTTCCCC | 448 |
| ITGA4 | | 11117 | CGGTGGTTGGGGCCTAGAAGCG | 481 |
| ITGA4 | | 11154 | CGCGCCCCTCGCTGTGACCGCCCAGCCCG | 524 |
| ITGA4 | | 11203 | CGGGGAGTGGGACTGCGGCGGGGAGCCG | 580 |
| ITGA4 | | 11208 | ACTCGCCGAAGGCCCCTGGGGAAC | 718 |
| ITGA4 | | 11222 | CGGGCTGCATGCGTGAGCAGG | 840 |
| ITGA4 | | 11252 | CGGCAGGCGGTTTAGGCTGTGGCTG | 885 |
| ITGA4 | | 11278 | CCGATTCGGATTGCTCCAGCTGG | 962 |
| ITGA4 | | 11289 | CGCACCCACTCAGTTGCCACGGG | 1008 |
| ITGA4 | | 11327 | CGGAGACCCACAACGCAACACACC | 1099 |
| APP | | 7607 | CGCGACCCTGCGCGGGGCACCG | 1 |
| APP | | 7741 | GTGCGAGTGGGATCCGCCGCG | 34 |
| APP | | 7875 | CGCGCCGCCACCGCCGCCGTCTCCCGG | 68 |
| APP | | 8009 | CGCGCACGCTCCTCCGCGTGCTCTCG | 101 |
| APP | | 8143 | CCGAGGAAACTGACGGAGCCCGAGCGCGG | 137 |
| APP | | 8145 | CGAGTCAGCTGATCCGGCCCACCCCG | 186 |
| APP | | 8310 | CGAGAGAGACCCCTAGCGGCGCCG | 221 |
| APP | | 8475 | CGCCCGCTCGCGCCGGGAGGGGCCCTCG | 256 |
| APP | | 8640 | CGCGCCCACAGGTGCACGCGCCCTTGGCG | 289 |
| APP | | 8805 | GGCCGACGGCCCACCTGGGCTTCG | 351 |
| APP | | 8825 | CGCTGAGGCTCTAGAAAAGTCGAGAG | 446 |
| APP | | 8843 | CTCGTCCCCGTGAGCTTGAATCATCCGACCC | 480 |
| APP | | 8912 | AGGCGTTTCTGGAAGAGAATGAGAACG | 604 |
| APP | | 8927 | CGTCAAAAGCAGGCACGAGCAACCTG | 701 |
| APP | | 8928 | GAACGAACCAAAGGAGCAAGGCG | 742 |
| APP | | 8929 | CGCTGACAAGGGTGCCTAGGCCCGG | 1318 |
| APP | | 8948 | CGCAATTCCGTATTTGTTCCGG | 1738 |
| APP | | 8969 | GTACGTTGGCAGACGCAGTGACG | 4923 |
| CMYC | | 7551 | CGATGAGGGTATTAACTCTGGC | 335580 |
| CMYC | CM12 | 7552 | CGGGGGTCCTCAGCCGTCCAGACC | 518 |
| CMYC | CM13 | 7602 | CGCTTATGGGGAGGGTGGGGAGGG | 634 |
| CMYC | CM14 | 7603 | CGGTGGGCGGAGATTAGCGAGAGA | 559 |
| CMYC | | 7606 | GGCGCTTATGGGGAGGGTGGGGAGGG | 632 |
| CMYC | | 13684 | CCTGGCACGTGTCCCTGGTCAAG | 3482 |
| CMYC | | 13703 | CACGTGCGGCCTGTCAAGAGATGA | 5926 |
| FGFR1 | | 13484 | CGAGCCAGGCAGGGCCCCTCGCAAGTG | 1850 |
| FGFR1 | | 13522 | GACGGATATGAGTCCAGAAGTTGCG | 1472 |
| FGFR1 | | 13535 | TAGCTGCGTGCAGTGGCGCGCGCCTGT | 4910 |
| FGFR1 | | 13561 | CCGCCTCGCCAGCTCCCGAGCGCGAGTT | 10239 |
| FGFR1 | | 13655 | CGCCTCCTCCCAGGTGTGGGCTGGCTGCAGACCG | 3067 |
| CD68 | | 13681 | CGAGAACATGGCTTTCCAGCGTCTG | 520 |
| ALK | | 11471 | CGCCGGAGGAGGCCGTTTACACTGC | 3 |
| ALK | | 11530 | CGTGCGCGCAAGTCTCTTGCTTTCC | 132 |
| ALK | | 11555 | CGCTCTCCGCGCCGAGTGCCGCGCC | 269 |
| ALK | | 11621 | CGCCTTTTGCGTTCCTTTTGGCTCC | 482 |
| ALK | | 11681 | CGCAGGCACTGGAGCGGCCCCGGCG | 701 |
| ALK | | 11794 | CGACCCTCCGAACAGAGGCGGCGGG | 851 |
| ALK | | 11825 | CGCGCTGCTGCCCGACCCACGCAGT | 1022 |
| ALK | | 11901 | CGGGTCCGACTTCGGAAAAACAGGT | 1313 |
| ALK | | 11923 | CGGCCTGTCGGGTAGCACAGGAGTT | 2022 |
| MSI2 | | 11989 | CGGTGACGTCACGCACCCCCGTGCG | 360 |
| MSI2 | | 12058 | CGGATACAATTACCCATATTGT | 1535 |
| MSI2 | | 12059 | GACTCAGTTGCTAACAACCATGAGCG | 10624 |
| MSI2 | | 12060 | CAGTTGCTAACAACCATGAGCG | 10628 |
| MSI2 | | 12061 | CATGAAAATTTCACCAAGTATAAATTAC | 10909 |
| MSI2 | | 12062 | CACCAAGTATAAATTACAGGTCT | 10920 |
| JAK2 | | 12063 | CGCACCAGTTTGTCCACGTCCAGTG | 1663 |
| JAK2 | | 12098 | GCCGTCACTGCCGACATAAGCACAGAC | 1811 |
| CCND1 | | 12098 | CGCTGCTACTGCGCCGACAGCCCTC | 133 |
| CCND1 | | 12242 | CGGCAGAATGGGCGCATTTCCAAGA | 612 |
| CCND1 | | 12287 | ACGCCACGAGGGCACCCACGGGCGGA | 637 |
| CCND1 | | 12332 | CGGTGACCGCGGCCTGGGCGGATGG | 2755 |
| CCND1 | | 12388 | CGGGACTCAGCGCGGCTGCGCGCCG | 2907 |
| BL9 | | 13682 | TGTCCACCTGAACACCTAGTCC | 2388 |

TABLE 4
Additional DNAi Sequences Used in Supporting Data (disclosed in
Pat. No.: 7,807,647)

| | | | | Location relative to |
|---|---|---|---|---|
| | | | | 5′ upstream region |
| Target | Design ID | Sequence ID | Sequence | from gene start site |
| KRAS | KR1 | 51 | CCCGGAGCGGGACCGGACCGCGG | 5923 |
| KRAS | KR2 | 52 | GCCGGACCCACGCGGCGGCCCGCC | 5856 |
| BCL2 | BL2 | 13724 | CACGCACGCGCATCCCCGCCCGTG | 2388 |
| BCL2 | BL3 | 13725 | ACCGGCGCTCGGCGCGCGGA | Mismatched |
| BCL2 | BL4 | 13726 | GACGCGCCGGGCCGGGCGGA | Mismatched |
| BCL2 | BL7 | 13727 | GGCGCGCGGGGCCGGGCCGGG | — |
| CMYC | CM7 | 13728 | GGGCGCCTCGCTAAGGCTGGGGAAAGGGCCGCGC | 969 |

TABLE 5
DNAi Sequences Used in Supporting Data as Negative Controls

| | | | | Location relative to 5′ upstream |
|---|---|---|---|---|
| Target | Design ID | SEQ ID NO: | Sequence | region from gene start site |
| Survivin | SU3 | 105 | GACATCGCTGTCCCGGCGAGTACATCGTT | 665 |
| KRAS | KR0525 | 1516 | AGTCTCCCCTTCCCGGAGACT | 10265 |

1. An oligonucleotide that hybridizes to a non-coding region in or upstream of a promoter for a target gene, wherein the oligonucleotide comprises:
a length of 20-34 bases;
at least one CG pair;
at least 40% C and G content;
no more than five consecutive bases of the same nucleotide; and
at least one secondary structure for said oligonucleotide.

2. The oligonucleotide of claim 1, wherein said oligonucleotide comprises a C and G content of at least 50%.

3. The oligonucleotide of claim 1, wherein said oligonucleotide comprises a C and G content from about 50 to 80%.

4. The oligonucleotide of claim 1, wherein said oligonucleotide comprises at least two CG pairs.

5. The oligonucleotide of claim 1, wherein said oligonucleotide hybridizes within a CG region, CpG island region, nuclease hypersensitive site, or CIS regulatory region.

6. The oligonucleotide of claim 1, wherein said non-coding region is located within a CG region, CpG island, nuclease hypersensitive site, or CIS regulatory region.

7. The oligonucleotide of claim 1, wherein said oligonucleotide is a reverse and full complement of a sense strand of said non-coding region of the target gene.

8. The oligonucleotide of claim 1, wherein said oligonucleotide is unique to the nucleotide sequence of the non-coding region.

9. The oligonucleotide of claim 1, wherein the nucleotide sequence of the non-coding region is not duplicated in a genome comprising the target gene.

10. The oligonucleotide of claim 1, wherein the nucleotide sequence of the non-coding region comprises less than 80% homology to other nucleotide sequences in a genome with a target gene.

11. The oligonucleotide of claim 1, wherein the nucleotide sequence of the non-coding region comprises less than 50% homology to other nucleotide sequences in a genome with a target gene.

12. The oligonucleotide of claim 1, wherein said oligonucleotide comprises at least four bases in a linear section of the secondary structure.

13. The oligonucleotide of claim 1, wherein said oligonucleotide comprises at least five bases in a linear section of the secondary structure.

14. The oligonucleotide of claim 1, wherein said oligonucleotide comprises at least one CG pair within the first 40% of the bases of said oligonucleotide.

15. The oligonucleotide of claim 1, wherein said oligonucleotide comprises at least one CG pair within the first 50% of the bases of said oligonucleotide.

16. The oligonucleotide of claim 1, wherein said oligonucleotide further comprises at least one CG pair that is prior to or in the nonlinear section of the secondary structure.

17. The oligonucleotide of claim 1, wherein said oligonucleotide comprises a linear section before a secondary structure, no oligonucleotides that extend beyond the secondary structure, and at least one CG pair within the linear section or the secondary structure.

18. The oligonucleotide of claim 1, wherein said oligonucleotide has a linear section before a secondary structure and no oligonucleotides that extend beyond the secondary structure

19. The oligonucleotide of claim 1, wherein said oligonucleotide does not comprise a single G or T base after the nonlinear section of the secondary structure.

20. The oligonucleotide of claim 1, wherein said secondary structure comprises at least one hairpin loop.

21. The oligonucleotide of claim 1, wherein said secondary structure comprises at least two hairpin loops.

22. The oligonucleotide of claim 19 or 20, wherein said secondary structure comprises at least three nucleotide bridges in the nonlinear section of the secondary structure.

23. The oligonucleotide of claim 1, wherein said oligonucleotide comprises a theoretical ΔG between −0.1 to −7.

24. The oligonucleotide of claim 23, wherein said theoretical ΔG is between −1 to −5.

25. The oligonucleotide of claim 1, wherein said oligonucleotide comprises a theoretical ΔTm between 30-70 degrees Celsius.

26. The oligonucleotide of claim 1, wherein said oligonucleotide begins at the 5′ end with the bases selected from CG, CGG, CGC, CGT, CGA, GCG, CCC, CCG, GTC, TCC, TCG, ACG, CAC, CAG, GAG, AGA, GAC, GAA, AGC, or GCC.

27. The oligonucleotide of claim 1, wherein said oligonucleotide ends at the 3′ end with the bases selected from CG, GCG, GGC, CGG, GCC, CGC, CCG, ACG, TCG, GGG, TGC, CCC, GTG, or CTC.

28. The oligonucleotide of claim 1, wherein said non-coding region is located less than 7000 bases upstream of the coding region of the target gene.

29. The oligonucleotide of claim 1, wherein said non-coding region is located less than 5000 bases upstream of the coding region of the target gene.

30. The oligonucleotide of claim 1, wherein said non-coding region is located less than 3000 bases upstream of the coding region of the target gene.

31. The oligonucleotide of claim 1, wherein said non-coding region is located less than 1000 bases upstream of the coding region of the target gene.

32. The oligonucleotide of claim 1, wherein said non-coding region is located less than 500 bases up- or downstream of a transcription factor binding site or translocation site of target gene.

33. The oligonucleotide of claim 1, wherein said non-coding region is located less than 100 bases up- or downstream of a transcription factor binding site or translocation site of target gene.

34. The oligonucleotide of claim 1, wherein said oligonucleotide does not comprise a CpG Coley motif.

35. The oligonucleotide of claim 1, wherein said oligonucleotide does not form a triplex structure.

36. The oligonucleotide of claim 1, wherein said oligonucleotide does not form a G-quadruplex structure.

37. The oligonucleotide of claim 1, wherein said oligonucleotide is a single stranded DNA.

38. The oligonucleotide of claim 1, wherein said oligonucleotide hybridizes to an Sp1 motif or transcription factor binding site.

39. The oligonucleotide of claim 1, wherein said target gene is selected from Survivin, Beclin-1, STAT3, HIF1A, IL-8, KRAS, MTTP, ApoC III, ApoB, IL-17, MMP2, FAP, P-selectin, IL-6, IL-23, AKT, CRAF, Beta-catenin, PCSK9, MEK1, MEK2, CD4, WNT1, Clusterin, NRAS, EZH2, HDAC1, PD-1, TNFα, MIF1, TTR, HBV, HAMP, ERBB2, PARP1, ITGA4, APP, FGFR1, CD68, ALK, MSI2, JAK2, CCND1, or selected from Table 2.

40. The oligonucleotide of claim 1, wherein said oligonucleotide is selected from the group consisting of any of the sequences disclosed in Table 3.

41. The oligonucleotides of claim 1, wherein said oligonucleotide hybridizes to a hot zone of a target gene.

42. The oligonucleotide of claim 1, wherein at least one of the cytosine bases in said oligonucleotide is 5-methylcytosine.

43. The oligonucleotide of claim 1, wherein at least one of the cytosine bases in said CG pair is 5-methylcytosine.

44. The oligonucleotide of claim 1, wherein all of said cytosine bases in said oligonucleotide are 5-methylcytosine.

45. The oligonucleotide of claim 1, wherein said hybridization of said oligonucleotide to the non-coding region modulates the target gene.

46. The oligonucleotide of claim 1, wherein said hybridization of said oligonucleotide to the non-coding region of the target gene modulates expression or transcription of said target gene.

47. The oligonucleotide of claim 1, wherein said hybridization of said oligonucleotide to the non-coding region of the target gene modulates a cell signaling pathway.

48. The oligonucleotide of claim 1, wherein said hybridization of said oligonucleotide to the non-coding region of said target gene produces phenotypic changes in a mammal.

49. The oligonucleotide of claim 1, wherein said hybridization of said oligonucleotide to the non-coding region of said target gene influences a non-gene target due to a chromosomal rearrangement.

50. The oligonucleotide of claim 1, wherein said target gene is on a chromosome of a cell, and wherein said hybridization of said oligonucleotide to said non-coding region reduces proliferation of said cell.

51. The oligonucleotide of claim 1, wherein said target gene is an oncogene.

52. A composition comprising an oligonucleotide according to any one of claims 1-51 and a pharmaceutically acceptable carrier.

53. The composition of claim 52, wherein the pharmaceutically acceptable carrier is a liposome.

54. The composition of claim 53, wherein the liposome is an amphoteric liposome.

55. The composition of claim 53, wherein the liposome comprises a neutral lipid.

56. The composition of claim 53, wherein the liposome comprises a mixture of neutral lipids and lipids with amphoteric properties, wherein the mixture of lipid components comprises anionic and cationic properties and at least one such component is pH responsive.

57. The composition according to any one of claims 52-56, wherein the composition further comprises an additional therapeutic agent.

58. The composition of claim 57, wherein the additional therapeutic agent is a second oligonucleotide, chemotherapeutic agent, immunotherapeutic agent, or radiotherapy.

59. The composition of claim 52, wherein said composition has two (2) therapeutic agents.

60. The composition of claim 59, wherein one therapeutic agent treats a cancer disease and the other therapeutic agent treats a non-cancer disease.

61. A method of inhibiting protein expressing in a cell with a target gene comprising introducing into said cell an oligonucleotide according to any one of claims 1-51 or composition according to any one of claims 52-60.

62. A method of mediating target-specific RNA in a mammalian cell in vitro, comprising contacting said mammalian cell in vitro with an oligonucleotide according to any one of claims 1-51 or composition according to any one of claims 52-60.

63. A method of mediating protein down regulation in a mammalian cell in vitro, comprising contacting said mammalian cell in vitro with an oligonucleotide according to any one of claims 1-51 or composition according to any one of claims 52-60.

64. A method of treating a patient having a disease characterized by the presence or undesired production of a protein implicated in said disease, comprising administering to said patient a pharmaceutically effective amount of an oligonucleotide according to any one of claims 1-51 or composition according to any one of claims 52-60.

65. A method of treating a patient having a disease characterized by the presence or undesired production of a protein implicated in said disease, comprising administering to said patient a pharmaceutically effective amount between 1 mg/m² and 500 mg/m² of an oligonucleotide according to any one of claims 1-51 or composition according to any one of claims 52-60.

66. A method of treating a mammal having a disease characterized by the presence or undesired production of a protein implicated in disease, comprising administering to said mammal a pharmaceutically effective amount of an oligonucleotide according to any one of claims 1-51 or composition according to the description and the compositions in any of claims 52-60.

67. A method of treating a plant having a disease characterized by the presence or undesired production of a protein implicated in disease, comprising introducing to said plant an effective amount of an oligonucleotide according to any one of claims 1-51 or composition according to the description and the compositions in any of claims 52-60.

68. A method of administration of a therapeutic disclosed herein and a oligonucleotide according to any one of claims 1-51 or a composition according to any one of claim 52-60, wherein said administration is through a route selected from oral, vapor, inhalation, dermal, subdermal, subcutaneous, parental, parenterally, ear, nose, nasally, bucally, eye, otic, ophthalmically, rectal, vaginal, suppository or implant, implanted reservoir, dermal, dermal skin patch, injection, or sub-lingual.

69. A method or kit for a diagnosis and treatment of a disease comprising the steps of administering to a patient a pharmaceutically effective amount of an oligonucleotides accordingly to any one of claims 1-51 or a composition according to any one of claims 52-60, wherein the patient is characterized by the presence of, or undesired production of, a protein implicated in said disease, and the method further comprising evaluating said patient for the presence of, or undesired production of said protein.

70. An single stranded DNA oligonucleotide that hybridizes to coding or non-coding region of a target gene, wherein the oligonucleotide comprises:
a length of 12-50 bases;
at least 30% C and G content; and
no more than seven consecutive bases of the same nucleotide.

71. The oligonucleotide of claim 70, wherein the nucleotide sequence of the non-coding region comprises less than 80% homology to other nucleotide sequences in a genome with a target gene.

72. The oligonucleotide of claim 70, wherein said oligonucleotide comprises at least one CG pair within the first 40% of the bases of said oligonucleotide.

73. The oligonucleotide of claim 70 further comprising a secondary structure.

74. The oligonucleotide of claim 70, wherein said oligonucleotide comprises a theoretical ΔG between −0.1 to −7.

75. The oligonucleotide of claim 70, wherein said oligonucleotide comprises a theoretical ΔTm between 30-70 degrees Celsius.

76. The oligonucleotide of claim 70, wherein said non-coding region is located less than 7000 bases upstream of the coding region of the target gene.

77. The oligonucleotide of claim 70, wherein said non-coding region is located less than 500 bases up- or downstream of a transcription factor binding site or translocation site of target gene.

78. The oligonucleotide of claim 70, wherein said non-coding region is located with a CG region, nuclease hypersensitive site, or CpG island of the genome comprising the target gene.

79. The oligonucleotide of claim 70, further comprises at least one CG pair and optionally at least one of the cytosine bases in said CG pair is 5-methylcytosine.

80. The oligonucleotide of claim 70, wherein said target gene is on a chromosome of a cell, and wherein said hybridization of said oligonucleotide reduces proliferation of said cell.

81. A composition comprising an oligonucleotide according to any one of claims 70-80 and a pharmaceutically acceptable carrier.

82. The composition of claim 81, wherein the pharmaceutically acceptable carrier is a liposome.

83. The composition according to any one of claim 81 or 82 wherein the composition further comprises an additional therapeutic agent.

84. A method of inhibiting or silencing gene transcription in a cell with a target gene comprising introducing into said cell an oligonucleotide according to any one of claims 70-80 or composition according to any one of claims 81-83.

85. A method of mediating target-specific RNA in a mammalian cell in vitro, comprising contacting said mammalian cell in vitro with an oligonucleotide according to any one of claims 70-80 or composition according to any one of claims 81-83.
